(19) United States
(12) Patent Application Publication
(10) Pub. No.: US 2015/0038421 A1
(43) Pub. Date: Feb. 5, 2015

(54) FACTOR VIII COMPOSITIONS AND METHODS OF MAKING AND USING SAME

(71) Applicant: Amunix Operating Inc., Mountain View, CA (US)

(72) Inventors: Volker SCHELLENBERGER, Palo Alto, CA (US); Pei-Yun Chang, Menlo Park, CA (US); Fatbardha Varfaj, Mountain View, CA (US); John Kulman, Belmont, MA (US); Tongyao Liu, Lexington, MA (US); Garabet G. Toby, North Reading, MA (US); Haiyan Jiang, Belmont, MA (US); Robert Peters, Needham, MA (US); Deping Wang, Sharon, MA (US); Baisong Mei, Waban, MA (US); Joshua Silverman, Sunnyvale, CA (US); Chia-Wei Wang, Milpitas, CA (US); Benjamin Spink, San Carlos, CA (US); Nathan Geething, San Juan, PR (US)

(21) Appl. No.: 14/317,888

(22) Filed: Jun. 27, 2014

Related U.S. Application Data
(63) Continuation of application No. 13/365,166, filed on Feb. 2, 2012, now abandoned.
Application No. 13/365,166 is a continuation of application No. PCT/US2011/048517, filed on Aug. 19, 2011.
(60) Provisional application No. 61/401,791, filed on Aug. 19, 2010.

(51) Int. Cl. C07K 14/755; C07K 14/00

(52) U.S. Cl. CPC C07K 14/755; C07K 14/001; C07K 2319/95; C07K 2319/31
USPC 514/14.1; 530/383; 536/23.4; 435/69.6

(57) ABSTRACT

The present invention relates to compositions comprising factor VIII coagulation factors linked to extended recombinant polypeptide (XTEN), isolated nucleic acids encoding the compositions and vectors and host cells containing the same, and methods of making and using such compositions in treatment of factor VIII-related diseases, disorders, and conditions.

### CROSS REFERENCE TO RELATED APPLICATION

This application is a continuation application of PCT Application No. PCT/US2011/48517, filed Aug. 19, 2011, which claims the benefit of U.S. Provisional Application Ser. No. 61/401,791 filed Aug. 19, 2010, all of which are incorporated herein by reference in their entirety.

### BACKGROUND OF THE INVENTION

Factor VIII is an important component of the intrinsic pathway of the blood coagulation cascade. In the circulation, factor VIII is mainly complexed to von Willebrand factor. Upon activation by thrombin, (Factor IIa), it dissociates from the complex to interact with factor IXa in the intrinsic coagulation cascade, which, in turn, activates factor X. Once removed from the von Willebrand factor complex, activated factor VIII is proteolytically inactivated by activated Protein C (APC), factor Xa, and factor IXa, and is quickly cleared from the blood stream. When complexed with normal von Willebrand factor protein, the half-life of factor VIII is approximately 12 hours, whereas in the absence of von Willebrand factor, the half-life of factor VIII is reduced to 2 hours (Tuddenham E G, et al., Br J Haematol. (1982) 52(2):259-267).

In hemophilia, the clotting of blood is disturbed by a lack of certain plasma blood clotting factors. Hemophilia A is a deficiency of factor VIII, and is a recessive sex-linked, X chromosome disorder that represents 80% of hemophilia cases. The standard of care for the management of hemophilia A is replacement therapy with recombinant factor VIII concentrates. Subjects with severe hemophilia A have circulating procoagulant factor VIII levels below 1-2% of normal, and are generally on prophylactic therapy with the aim of keeping factor VIII above 1% between doses, which can usually be achieved by giving factor VIII two to three times a week. Persons with moderately severe hemophilia (factor VIII levels of 2-5% of normal) constitute 25-30% hemophilia incidents and manifest bleeding after minor trauma. Persons with mild hemophilia A (factor VIII levels of 5-40% of normal) comprise 15-20% of all hemophilia incidents, and develop bleeding only after significant trauma or surgery.

The in vivo activity of exogenously supplied factor VIII is limited both by a short protein half-life and inhibitors that bind to the factor VIII and diminish or destroy hemostatic function. As such, frequent injections of factor VIII are required. Large proteins such as factor VIII are normally given intravenously so that the medicament is directly available in the blood stream. In addition, it has been previously demonstrated that an unmodified factor VIII injected intramuscularly yielded a maximum circulating level of only 1.4% of the normal plasma level (Pool et al, New England J. Medicine, vol. 275, no. 10, p. 547-548, 1966).

Chemical modifications to a therapeutic protein can modify its in vivo clearance rate and subsequent serum half-life. One example of a common modification is the addition of a polyethylene glycol (PEG) moiety, typically coupled to the protein via an aldehyde or N-hydroxysuccinimide (NHS) group on the PEG reacting with an amine group (e.g. lysine side chain or the N-terminus). However, the conjugation step can result in the formation of heterogeneous product mixtures that require extraction, purification and/or other further processes, all of which inevitably affect product yield and quality control. Also, the pharmacologic function of coagulation factors may be hampered if amino acid side chains in the vicinity of its binding site become modified by the PEGylation process. Other approaches include the genetic fusion of an Fc domain to the therapeutic protein, which increases the size of the therapeutic protein, hence reducing the rate of clearance through the kidney. In some cases, the Fc domain confers the ability to bind to, and be recycled from lysosomes by the FcRn receptor, resulting in increased pharmacokinetic half-life. Unfortunately, the Fc domain does not fold efficiently during recombinant expression, and tends to form insoluble precipitates known as inclusion bodies. These inclusion bodies must be solubilized and functional protein must be renatured from the misfolded aggregate, which is a time-consuming, inefficient, and expensive process.

### SUMMARY OF THE INVENTION

The present invention relates to novel coagulation factor VIII fusion protein compositions and the uses thereof. Specifically, the compositions provided herein are particularly used for the treatment or improvement of a condition associated with hemophilia A, deficiencies of factor VIII, bleeding disorders and coagulopathies. In one aspect, the present invention provides compositions of isolated fusion proteins comprising a factor VIII (FVIII) and one or more extended recombinant polypeptides (XTEN). A subject XTEN useful for constructing such fusion proteins is typically a polypeptide with a non-repetitive sequence and unstructured conformation. In one embodiment, one or more XTEN is linked to a coagulation factor FVIII (“CF”) selected from native factor VIII, factor VIII B-domain deleted sequences (“FVIII BDD”), and sequence variants thereof (all the foregoing collectively “FVIII” or “CF”), resulting in a coagulation factor VIII-XTEN fusion protein (“CFXTEN”). In an embodiment, the isolated fusion protein comprises a factor VIII polypeptide that comprises an A1 domain, an A2 domain, an A3 domain, and a C1 domain. In another embodiment, the factor VIII polypeptide further comprises a B domain or a portion thereof, an a3 domain, and a C2 domain. In another embodiment, the present disclosure is directed to pharmaceutical compositions comprising the fusion proteins and the uses thereof for treating, e.g., factor VIII-related diseases, or conditions. The CFXTEN compositions have enhanced pharmacokinetic properties compared to FVIII not linked to XTEN, which may permit more convenient dosing and improved efficacy. In yet another embodiment, the CFXTEN compositions of the invention do not have a component selected the group consisting of: polyethylene glycol (PEG), albumin, antibody, and an antibody fragment.

In an embodiment, the invention provides an isolated fusion protein comprising a factor VIII polypeptide and at least one extended recombinant polypeptide (XTEN), wherein said at least one XTEN is linked to the factor VIII polypeptide at one or more locations. For example, the at least one XTEN is linked to one or more locations selected from the C-terminus of said factor VIII polypeptide, within the A1 domain of said factor VIII polypeptide; within the A2 domain of said factor VIII polypeptide, within the A3 domain of said factor VIII polypeptide; within the B domain of the factor VIII polypeptide, within the C1 domain of said factor VIII polypeptide; at one or more location between any two adjacent domains of said factor VIII polypeptide (for example, between the A1 and A2 domains, the A2 and B domains, the B and a3 domains, the a3 and A3 domains, the A2 and a3 domains when the B domain is completely deleted, the A2 and A3 domains, and the A3 and C1 domains, the C1 and C2 domains or any combination thereof); at the N-terminus of said factor VIII polypeptide; at one or more insertion locations from FIG. 5; at one or more insertion locations from Table 5; at one or more insertion locations from Table [23], and/or any combination thereof. In an embodiment, In an embodiment, the XTEN is characterized in that: the XTEN comprises at least 36, or at least 42, or at least 72, or at least 96, or at least 144, or at least 288, or at least 400, or at least 500, or at least 576, or at least 600, or at least 700, or at least 800, or at least 864, or at least 900, or at least 1000, or at least 2000, to about 3000 amino acid residues or even more residues; the sum of glycine (G), alanine (A), serine (S), threonine (T), glutamate (E) and proline (P) residues constitutes at least about 80%, or at least about 90%, or at least about 95%, or at least about 96%, or at least about 97%, or at least about 98%, or at least about 99% of the total amino acid residues of the XTEN; the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14, or about 12 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10; the XTEN has greater than 90%, or greater than 95%, or greater than 99% random coil formation as determined by GOR algorithm; the XTEN has less than 2% alpha helices and 2% beta-sheets as determined by Chou-Fasman algorithm; the XTEN lacks a predicted T-cell epitope when analyzed by TEPITOPE algorithm, wherein the TEPITOPE threshold score for said prediction by said algorithm has a threshold of −9, and wherein said fusion protein exhibits a terminal half-life that is longer than at least about 12 h, or at least about 24 h, or at least about 48 h, or at least about 72 h, or at least about 96 h, or at least about 120 h, or at least about 144 h, or at least about 21 days or greater. In one embodiment, the isolated fusion protein comprises at least another XTEN, which can be identical or different to the first XTEN, In one embodiment, the at least another XTEN is linked to the factor VIII polypeptide at one or more locations. For example, the at least another XTEN is linked to one or more locations selected from the C-terminus of said factor VIII polypeptide, within the A1 domain of said factor VIII polypeptide; within the A2 domain of said factor VIII polypeptide, within the A3 domain of said factor VIII polypeptide; within the B domain of the factor VIII polypeptide, within the C1 domain of said factor VIII polypeptide; at one or more location between any two adjacent domains of said factor VIII polypeptide (for example, between the A1 and A2 domains, the A2 and B domains, the B and a3 domains, the a3 and A3 domains, the A2 and a3 domains when the B domain is completely deleted, the A2 and A3 domains, and the A3 and C1 domains, the C1 and C2 domains or any combination thereof); at the N-terminus of said factor VIII polypeptide; at one or more insertion locations from FIG. 5; at one or more insertion locations from Table 5; at one or more insertion locations from Table [23], and/or any combination thereof. In another embodiment of the isolated fusion protein, the at least another XTEN is linked to the factor VIII polypeptide at the C-terminus of the factor VIII polypeptide, In another embodiment of the isolated fusion protein, the at least another XTEN is linked within the B domain of said factor VIII polypeptide. In some embodiments, the at least another XTEN is linked within the B domain within the sequence SFSQNPPVLKRHQR. In one embodiment of the foregoing, the at least another XTEN is linked between the S and Q residues of the sequence SFSQNPPVLKRHQR. In another embodiment of the foregoing, the at least another XTEN is linked between the N and P residues of the sequence SFSQNPPVLKRHQR. In another embodiment, the isolated fusion protein comprises FVIII and multiple XTEN sequences which are inserted within the B domain and to the N-terminus and/or the C-terminus of the factor VIII polypeptide. In another embodiment, the isolated fusion protein comprising FVIII and multiple XTEN sequences, one of which is linked to the N-terminus and/or the C-terminus of the factor VIII polypeptide and another is inserted within the B domain of the factor VIII polypeptide, such insertion takes place at the C-terminal end of about amino acid residue number 740 to about 745 (or alternatively about amino acid residue number 741 to about 743 of the B-domain) of the B-domain and to the N-terminal end of amino acid residue numbers 1640 to about 1689 (or alternatively about 1638 to about 1648 of the B-domain) of the B-domain of a native FVIII sequence. The resulting fusion protein has a cumulative length of the XTEN portion in the range of at least about 100 to about 3000 amino acid residues. In another embodiment, the isolated fusion protein comprises at least a second XTEN, which may be identical or different to the first XTEN, wherein said at least second XTEN is linked to said factor VIII polypeptide at one or more locations selected from the following: i) at or within 6 amino acids to the N- or C-terminus side of an insertion location from Table 5 or Table 25 or as illustrated in FIG. 7; ii) a location between any two adjacent domains of said factor VIII polypeptide, wherein said two adjacent domains are selected from the group consisting of A1 and A2 domains, A2 and B domains, B and A3 domains, A3 and C1 domains, and C1 and C2 domains; iii) the N-terminus of said factor VIII polypeptide; and the C-terminus of said factor VIII polypeptide. In the foregoing embodiments, the at least second XTEN can have the same characteristic as the first XTEN. For example, the second XTEN is characterized in that: the XTEN comprises at least 36, or at least 42, or at least 72, or at least 96, or at least 144, or at least 288, or at least 400, or at least 500, or at least 576, or at least 600, or at least 700, or at least 800, or at least 864, or at least 900, or at least 1000, or at least 2000, to about 3000 amino acid residues; the sum of glycine (G), alanine (A), serine (S), threonine (T), glutamate (E) and proline (P) residues constitutes at least about 80%, or at least about 90%, or at least about 95%, or at least about 96%, or at least about 97%, or at least about 98%, or at least about 99%, of the total amino acid residues of the XTEN; the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14, or about 12 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10; the XTEN has greater than 90%, or greater than 95%, or greater than 99%, random coil formation as determined by GOR algorithm; the XTEN has less than 2% alpha helices and 2% beta-sheets as determined by Chou-Fasman algorithm; the XTEN lacks a predicted T-cell epitope when analyzed by TEPITOPE algorithm, wherein the TEPITOPE threshold score for said prediction by said algorithm has a threshold of −9. In some embodiments, the XTEN of the fusion proteins are further characterized in that the sum of asparagine and glutamine residues is less than 10%, or less than 5%, or less than 2% of the total amino acid sequence of the XTEN, the sum of methionine and tryptophan residues is less than 2% of the total amino acid sequence of the XTEN, and the XTEN has less than 5% amino acid residues with a positive charge. In one embodiment, the fusion proteins of this paragraph comprise one or more XTEN having at least 80%, or at least about 90%, or at least about 95%, or at least about 96%, or at least about 97%, or at least about 98%, or at least about 99% or sequence identity compared to a sequence of comparable length selected from any one of Table 4, Table 9, Table 10, Table 11, Table 12, and Table 13, when optimally aligned.

In one embodiment, the isolated fusion protein comprises a FVIII polypeptide having at least 80% sequence identity, or at least about 90%, or about 95%, or about 96%, or about 97%, or about 98%, or about 99% sequence identity compared to an amino acid sequence selected from Table 1, when optimally aligned. In one embodiment, the FVIII polypeptide of the isolated fusion protein comprises human FVIII. In another embodiment, the FVIII polypeptide of the fusion protein comprises a B-domain deleted (BDD) variant of human FVIII.

In one embodiment, the isolated fusion protein that comprises a factor VIII and one or more XTEN exhibits an apparent molecular weight factor of at least about 1.3, or at least about two, or at least about three, or at least about four, or at least about five, or at least about six, or at least about seven, or at least about eight, or at least about nine, or at least about 10, when measured by size exclusion chromatography or comparable method.

In an embodiment, the isolated fusion protein comprises a factor VIII polypeptide that is linked to an XTEN described herein via one or two cleavage sequences that each is cleavable by a protease selected from the group consisting of factor XIa, factor XIIa, kallikrein, factor VIIa, factor IXa, factor Xa, factor IIa (thrombin), elastase-2, MMP-12, MMP13, MMP-17, MMP-20, or a protease of Table 7 wherein cleavage at the cleavage sequence by the protease releases the factor VIII sequence from the XTEN sequence and wherein the released factor VIII sequence exhibits an increase in procoagulant activity of at least about 30%, or at least about 40%, or at least about 50%, or at least about 60%, or at least about 70%, or at least about 80%, or at least about 90% compared to the uncleaved fusion protein. In one embodiment, the isolated fusion protein comprising factor VIII and one or more XTEN linked with one or more integrated cleavage sequences has a sequence having at least about 80% sequence identity compared to a sequence from Table 30, alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared to a sequence from Table 30, when optimally aligned. However, the invention also provides substitution of any of the FVIII sequences of Table 1 or Table 31 for a FVIII in a sequence of Table 30, and substitution of any XTEN sequence of Table 4 for an XTEN in a sequence of Table 30, and substitution of any cleavage sequence of Table 7 for a cleavage sequence in a sequence of Table 30. In embodiments having the subject cleavage sequences linking the FVIII to the XTEN, cleavage of the cleavage sequence by the protease releases the XTEN from the fusion protein. In one embodiment, wherein the fusion protein is in the presence of proteases capable of cleaving the cleavage sequence and activating FVIII, the cleavage of the cleavage sequence linking XTEN to FVIII occurs prior to or concomitant with activation of FVIII. In some embodiments of the fusion proteins comprising cleavage sequences that link XTEN to FVIII, the FVIII component becomes active or has an increase in activity upon its release from the XTEN by cleavage of the cleavage sequence, wherein the resulting procoagulant activity of the cleaved protein is at least about 30%, or at least about 40%, or at least about 50%, or at least about 60%, or at least about 70%, or at least about 80%, or at least about 90% compared to the corresponding FVIII not linked to XTEN. In other embodiments, the fusion protein comprises XTEN linked to the FVIII by a cleavage sequence that is cleavable by a procoagulant protease that does not activate a wild type factor VIII, wherein upon cleavage of the cleavage sequence, the XTEN is released from the fusion protein. In one embodiment of the foregoing, the cleavage sequence is cleavable by activated factor XI. In another embodiment, the fusion protein comprises XTEN linked to the FVIII by two heterologous cleavage sequences that are cleavable by different proteases, which can be sequences selected from Table 7. In a preferred embodiment, the cleavage sequence is cleavable by factor XIa, wherein the XIa protease is capable of cleaving the XTEN from the fusion protein.

In other embodiments, the isolated CFXTEN fusion proteins comprise two, three, four, five, six or more XTEN (each characterized as described above) linked to the FVIII. In the foregoing, each XTEN, which can be identical or can be different, comprises at least 36 to about 400, or 800, or 1000, or 1500, or 2000 to about 3000 amino acids and the cumulative length of the XTEN sequences is at least about 100 to about 3000, or about 200 to about 2000, or about 400 to about 1500, or about 800 to about 1200 amino acid residues. In one embodiment of the CFXTEN with two or more XTEN, each XTEN has at least 80% sequence identity, or at least about 90%, or about 95%, or about 96%, or about 97%, or about 98%, or about 99% sequence identity to a sequence of comparable length selected from any one of Table 4, Table 9, Table 10, Table 11, Table 12, or Table 13, when optimally aligned. In the foregoing embodiments with two or more XTEN, the fusion proteins exhibit an apparent molecular weight factor of at least about 1.3, or at least about 2, or at least about 3, or at least about 4, or at least about 5, or at least about 6, or at least about 7, or at least about 8, or at least about 9 or at least about 10 when measured by size exclusion chromatography or comparable method. In the isolated fusion proteins of the foregoing embodiments with two or more XTEN, the XTEN are linked to the factor VIII at different locations selected from insertion locations from Table 5 or Table 25 or as illustrated in FIG. 7, or between any two adjacent domains in the factor VIII sequence wherein said two adjacent domains are selected from the group consisting of A1 and A2, A2 and B, B and A3, A3 and C1, and C1 and C2, or the N-terminus of the factor VIII sequence, or the C-terminus of the factor VIII sequence.

The isolated fusion proteins of the embodiments comprising at least one, two, three, four, five, six, or more XTEN sequences exhibit a prolonged half-life as compared to a corresponding factor VIII polypeptide lacking said XTEN. In one embodiment, the isolated fusion proteins exhibit a serum degradation half-life that is at least two-fold, or three-fold, or four-fold, or five-fold longer than a factor VIII polypeptide lacking said XTEN. In another embodiment, the isolated fusion proteins exhibit a terminal half-life that is longer than about 24, or about 48, or about 72, or about 96, or about 120, or about 144, or about 168 hours or more when administered to a subject.

Non-limiting embodiments of fusion proteins with a single FVIII linked to a single XTEN are presented in Tables 14 and 28. In one embodiment, the invention provides a fusion protein composition has at least about 80% sequence identity compared to a sequence from Tables 14 or 28, alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared to a sequence from Tables 14 or 28. Non-limiting embodiments of fusion proteins with a single FVIII with one or more XTEN linked internally or terminal to the FVIII sequence are presented in Tables 14 and 29. In one embodiment, the invention provides a fusion protein composition that has at least about 80% sequence identity compared to a sequence from Table 14 or Table 29, alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared to a sequence from Table 14 or 29. In the embodiments of this paragraph, the invention further contemplates substitution of a different FVIII from Table 1 or Table 31 for the FVIII of any listed sequence, and a different XTEN from Tables 4 or 9-12 for an XTEN of any listed sequence.

The invention provides that the fusion proteins of the embodiments, with FVIII and XTEN characterized as described above, can be in different N- to C-terminus configurations. In one embodiment of the fusion protein composition, the invention provides a fusion protein of formula I:

(CF)-(XTEN) I

wherein independently for each occurrence, CF is a factor VIII as described herein and XTEN is an extended recombinant polypeptide wherein the XTEN comprises at least 36 to about 3000 amino acid residues, the sum of glycine (G), alanine (A), serine (S), threonine (T), glutamate (E) and proline (P) residues constitutes at least about 80%, or at least about 90%, or at least about 95%, or at least about 99% of the total amino acid residues of the XTEN; the XTEN is substantially non-repetitive such that (i) the XTEN contains no three contiguous amino acids that are identical unless the amino acids are serine; (ii) at least about 80%, or at least about 90%, or at least about 95%, or at least about 99% of the XTEN sequence consists of non-overlapping sequence motifs, each of the sequence motifs comprising about 9 to about 14, or about 12 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10, the XTEN has greater than 90%, or greater than 95%, or greater than 99% random coil formation as determined by GOR algorithm; the XTEN has less than 2% alpha helices and 2% beta-sheets as determined by Chou-Fasman algorithm; and the XTEN lacks a predicted T-cell epitope when analyzed by TEPITOPE algorithm, wherein the TEPITOPE threshold score for said prediction by said algorithm has a threshold of −9. In one embodiment, the XTEN exhibits at least about 80%, or at least about 90%, or at least about 95%, or at least about 99% sequence identity to a sequence of comparable length from any one of Table 4, Table 9, Table 10, Table 11, Table 12, and Table 13, when optimally aligned.

In another embodiment of the fusion protein composition, the invention provides a fusion protein of formula II:

(XTEN)ₓ-(S)ₓ-(CF)-(XTEN)_{y} II

wherein independently for each occurrence, CF is a factor VIII as described herein; S is a spacer sequence having between 1 to about 50 amino acid residues that can optionally include a cleavage sequence from Table 7 or amino acids compatible with restrictions sites; x is either 0 or 1; and XTEN is an extended recombinant polypeptide as described herein, e.g., as for formula I, and wherein the fusion protein comprises two XTENs, the XTENs are identical or different and the cumulative length of the XTENs is between about 100 to about 3000, or between 200 to about 2000, or between 400 to about 1000 amino acid residues.

In another embodiment of the fusion protein composition, the invention provides an isolated fusion protein, wherein the fusion protein is of formula III:

(XTEN)_{w}-(S)ₓ-(CF)-(S)_{y}-(XTEN)_{z} III

wherein independently for each occurrence, CF is a factor VIII; S is a spacer sequence having between 1 to about 50 amino acid residues that can optionally include a cleavage sequence from Table 7 or amino acids compatible with restrictions sites wherein for each occurrence, if there is any, the sequence of the spacer can be the same or different; w is either 0 or 1; x is either 0 or 1; y is either 0 or 1 wherein w+x+y+z≧1; and XTEN is an extended recombinant polypeptide as described herein, e.g., as for formula I, and wherein the fusion protein comprises two XTENs, the XTENs are identical or different and the cumulative length of the XTENs is between about 100 to about 3000, or between 200 to about 2000, or between 400 to about 1000 amino acid residues. In one embodiment of formula VII, the spacer sequence is GPEGPS. In another embodiment of formula VII, the spacer sequence is a sequence from Table 6.

In another embodiment of the fusion protein composition, the invention provides an isolated fusion protein of formula IV:

(A1)-(XTEN)ᵤ-(A2)-(XTEN)ᵥ-(B)-(XTEN)_{w}-(A3)-(XTEN)ₓ-(C1)-(XTEN)_{y}-(C2) IV

wherein independently for each occurrence, A1 is an A1 domain of FVIII; A2 is an A2 domain of FVIII; A3 is an A3 domain of FVIII; B is a B domain of FVIII which can be a fragment or a splice variant of the B domain; C1 is a C1 domain of FVIII; C2 is a C2 domain of FVIII; u is either 0 or 1; v is either 0 or 1; x is either 0 or 1; y is either 0 or 1 with the proviso that u+v+w+x+y≧1; and XTEN is an extended recombinant polypeptide as described herein, e.g., as for formula I, and wherein the fusion protein comprises at least two XTENs, the XTENs are identical or different and the cumulative length of the XTENs is between about 100 to about 3000, or between 200 to about 2000, or between 400 to about 1000 amino acid residues.

In another embodiment of the fusion protein composition, the invention provides an isolated fusion protein of formula V:

(XTEN)ₜ-(S)ₐ-(A1)-(S)_{b}-(XTEN)ᵤ-(S)_{b}-(A2)-(S)_{c}-(XTEN)ᵥ-(S)_{c}-(B)-(S)_{d}-(XTEN)_{w}-(S)_{d}-(A3)-(S)ₑ-(XTEN)ₓ-(S)ₑ-(C1)-(S)_{f}-(XTEN)_{y}-(S)_{f}-(C2)-(S)_{g}-(XTEN)_{z} V

wherein independently for each occurrence, A1 is an A1 domain of FVIII; A2 is an A2 domain of FVIII; A3 is an A3 domain of FVIII; B is a B domain of FVIII which can be a fragment or a splice variant of the B domain; C1 is a C1 domain of FVIII; C2 is a C2 domain of FVIII; S is a spacer sequence having between 1 to about 50 amino acid residues that can optionally include a cleavage sequence from Table 7 or amino acids compatible with restrictions sites wherein for each occurrence, if there is any, the sequence of the spacer can be the same or different; a is either 0 or 1; b is either 0 or 1; c is either 0 or 1; d is either 0 or 1; e is either 0 or 1; f is either 0 or 1; g is either 0 or 1; t is either 0 or 1; u is either 0 or 1; v is either 0 or 1; w is 0 or 1, x is either 0 or 1; y is either 0 or 1; z is either 0 or 1 with the proviso that t+u+v+w+x+y+z≧1; and XTEN is an extended recombinant polypeptide as described herein, e.g., as for formula I, and wherein the fusion protein comprises at least two XTENs, the XTENs are identical or different and the cumulative length of the XTENs is between about 100 to about 3000, or between 200 to about 2000, or between 400 to about 1000 amino acid residues. In another embodiment of the foregoing formula V, the fusion protein comprises at least two spacer sequences, each of which comprises a cleavage sequence that is cleavable by the same or different procoagulant proteases capable of cleaving one or more sequences selected from Table 7. In one embodiment of formula V, the spacer sequence is GPEGPS. In another embodiment of formula V, the spacer sequence is a sequence from Table 6.

In another embodiment of the CFXTEN composition, the invention provides an isolated fusion protein of formula VI:

(XTEN)ᵤ-(S)ₐ-(A1)-(S)_{b}-(XTEN)ᵥ-(S)_{b}-(A2)-(S)_{c}-(XTEN)_{w}-(S)_{c}-(A3)-(S)_{d}-(XTEN)ₓ-(S)_{d}-(C1)-(S)ₑ-(XTEN)_{y}-(S)ₑ-(C2)-(S)_{f}-(XTEN)_{z} VI

wherein independently for each occurrence, A1 is an A1 domain of FVIII; A2 is an A2 domain of FVIII; A3 is an A3 domain of FVIII; C1 is a C1 domain of FVIII; C2 is a C2 domain of FVIII; S is a spacer sequence having between 1 to about 50 amino acid residues that can optionally include a cleavage sequence from Table 7 or amino acids compatible with restrictions sites wherein for each occurrence, if there is any, the sequence of the spacer can be the same or different; a is either 0 or 1; b is either 0 or 1; c is either 0 or 1; d is either 0 or 1; e is either 0 or 1; f is either 0 or 1; u is either 0 or 1; v is either 0 or 1; w is 0 or 1, x is either 0 or 1; y is either 0 or 1; z is either 0 or 1 with the proviso that u+v+w+x+y+z≧1; and XTEN is an extended recombinant polypeptide as described herein, e.g., as for formula I, and wherein the fusion protein comprises at least two XTENs, the XTENs are identical or different and the cumulative length of the XTENs is between about 100 to about 3000, or between 200 to about 2000, or between 400 to about 1000 amino acid residues. In one embodiment of formula VI, the spacer sequence is GPEGPS. In another embodiment of formula VI, the spacer sequence is a sequence from Table 6.

In another embodiment of the CFXTEN composition, the invention provides an isolated fusion protein of formula VII:

(SP)-(XTEN)ₓ-(CS)ₓ-(S)ₓ-(FVIII_{—}1-745)-(S)_{y}-(XTEN)_{y}-(S)_{y}-(FVIII_{—}1640-2332)-(S)_{z}-(CS)_{z}-(XTEN)_{z} VIIa or

(SP)-(XTEN)ₓ-(CS)ₓ-(S)ₓ-(FVIII_{—}1-743)-(S)_{y}-(XTEN)_{y}-(S)_{y}-(FVIII_{—}1638-2332)-(S)_{z}-(CS)_{z}-(XTEN)_{z} VIIb

wherein independently for each occurrence, SP is a signal peptide with sequence MQIELSTCFFLCLLRFCFS, CS is a cleavage sequence listed in Table 7, S is a spacer sequence having between 1 to about 50 amino acid residues that can optionally include amino acids compatible with restrictions sites wherein for each occurrence, if there is any, the sequence of the spacer can be the same or different; “FVIII_{—}1-745” is residues 1-745 of Factor FVIII and “FVIII_{—}1640-2332” is residues 1640-2332 of FVIII, or “FVIII_{—}1-743” is residues 1-743 of Factor FVIII and “FVIII_{—}1638-2332” is residues 1638-2332 of FVIII; x is either 0 or 1, y is either 0 or 1, and z is either 0 or 1, wherein x+y+z>2; and XTEN is an extended recombinant polypeptide as described herein, e.g., as for formula I, and wherein the fusion protein comprises at least two XTENs, the XTENs are identical or different and the cumulative length of the XTENs is between about 100 to about 3000, or between 200 to about 2000, or between 400 to about 1000 amino acid residues. In one embodiment of formula VII, the spacer sequence is GPEGPS. In another embodiment of formula VII, the spacer sequence is a sequence from Table 6.

In another embodiment of the CFXTEN composition, the invention provides an isolated fusion protein of formula VIII:

(XTEN)ᵤ-(S)ₐ-(A1)-(S)_{b}-(XTEN)ᵥ-(S)_{b}-(A2)-(B1)-(S)_{c}-(XTEN)_{w}-(S)_{c}-(B2)-(A3)-(S)_{d}-(XTEN)ₓ-(S)_{d}-(C1)-(S)ₑ-(XTEN)_{y}-(S)ₑ-(C2)-(S)_{f}-(XTEN)_{z} FVIII

wherein independently for each occurrence, A1 is an A1 domain of FVIII; A2 is an A2 domain of FVIII; B1 is a fragment of the B domain that can have from residues to 740 to residues 745 (or alternatively from residues 741 to residues 743) of a native mature FVIII; B2 is a fragment of the B domain that can have from residues 1640 to 1689 (or alternatively from residues 1638 to 1648) of a native mature FVIII; A3 is an A3 domain of FVIII; C1 is a C1 domain of FVIII; C2 is a C2 domain of FVIII; S is a spacer sequence having between 1 to about 50 amino acid residues that can optionally include a cleavage sequence from Table 7 or amino acids compatible with restrictions sites, wherein for each occurrence, if there is any, the sequence of the spacer can be the same or different; a is either 0 or 1; b is either 0 or 1; c is either 0 or 1; d is either 0 or 1; e is either 0 or 1; f is either 0 or 1; u is either 0 or 1; v is either 0 or 1; w is 0 or 1, x is either 0 or 1; y is either 0 or 1; z is either 0 or 1 with the proviso that u+v+w+x+y+z≧1; and XTEN is an extended recombinant polypeptide wherein the XTEN comprises at least 36 to about 3000 amino acid residues, the sum of glycine (G), alanine (A), serine (S), threonine (T), glutamate (E) and proline (P) residues constitutes at least about 80%, or at least about 90%, or at least about 95%, or at least about 99% of the total amino acid residues of the XTEN; the XTEN is substantially non-repetitive such that (i) the XTEN contains no three contiguous amino acids that are identical unless the amino acids are serine; (ii) at least about 80%, or at least about 90%, or at least about 95%, or at least about 99% of the XTEN sequence consists of non-overlapping sequence motifs, each of the sequence motifs comprising about 9 to about 14, or about 12 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10, the XTEN has greater than 90%, or greater than 95%, or greater than 99% random coil formation as determined by GOR algorithm; the XTEN has less than 2% alpha helices and 2% beta-sheets as determined by Chou-Fasman algorithm; and the XTEN lacks a predicted T-cell epitope when analyzed by TEPITOPE algorithm, wherein the TEPITOPE threshold score for said prediction by said algorithm has a threshold of −9. In one embodiment, the XTEN exhibits at least about 80%, or at least about 90%, or at least about 95%, or at least about 99% sequence identity to a sequence of comparable length from any one of Table 4, Table 9, Table 10, Table 11, Table 12, and Table 13, when optimally aligned, and wherein the fusion protein comprises at least two XTENs, the XTENs are identical or different and the cumulative length of the XTENs is between about 100 to about 3000, or between 200 to about 2000, or between 400 to about 1000 amino acid residues. In one embodiment of formula VIII, the spacer sequence is GPEGPS. In another embodiment of formula VIII, the spacer sequence is a sequence from Table 6.

The fusion protein compositions in the configurations of formulae I-VIII and any other configuration disclosed herein exhibit an increased apparent molecular weight as determined by size exclusion chromatography, compared to the actual molecular weight. In some embodiments the fusion protein comprising a FVIII and one or more XTEN exhibits an apparent molecular weight of at least about 200 kD, or at least about 400 kD, or at least about 500 kD, or at least about 700 kD, or at least about 1000 kD, or at least about 1400 kD, or at least about 1600 kD, or at least about 1800 kD, or at least about 2000 kD, while the actual molecular weight of the FVIII component of the fusion protein is about 150 kDa in the case of a FVIII BDD, is about 265 kDa for the mature form of full-length FVIII, and the actual molecular weight of the fusion protein for a FVIII BDD plus a single XTEN ranges from about 200 to about 270 kDa. Accordingly, the fusion proteins comprising one or more XTEN configured as formulae I-VIII have an apparent molecular weight that is about 1.3-fold greater, or about 2-fold greater, or about 3-fold greater or about 4-fold greater, or about 8-fold greater, or about 10-fold greater, or about 12-fold greater, or about 15-fold greater than the actual molecular weight of the fusion protein. Further, the isolated fusion proteins configured as formulae exhibit an apparent molecular weight factor under physiologic conditions that is greater than about 1.3, or about 2, or about 3, or about 4, or about 5, or about 6, or about 7, or about 8, or about 10, or greater than about 15, as determined by size exclusion chromatography.

The fusion protein compositions of the embodiments and in the configurations of formulae I-VIII described herein are evaluated for retention of activity (including after cleavage of any incorporated XTEN-releasing cleavage sites) using any appropriate in vitro assay disclosed herein (e.g., the assays of Table 27 or the assays described in the Examples), to determine the suitability of the configuration for use as a therapeutic agent in the treatment of a coagulation-factor related disease, disorder or condition. In one embodiment, the CFXTEN fusion protein exhibits at least about 30%, or at least about 40%, or at least about 50%, or at least about 60%, or at least about 70%, or at least about 80%, or at least about 90% of the procoagulant activity compared to the FVIII not linked to XTEN. In another embodiment, the FVIII component released from the fusion protein by enzymatic cleavage of the incorporated cleavage sequence(s) linking the FVIII and XTEN components exhibits at least about 30%, or at least about 40%, or at least about 50%, or at least about 60%, or at least about 70%, or at least about 80%, or at least about 90% of the procoagulant activity compared to the FVIII not linked to XTEN.

In some embodiments, fusion proteins comprising FVIII and one or more XTEN and in one of the configurations of formulae I-VIII exhibit enhanced pharmacokinetic properties compared to FVIII not linked to XTEN, wherein the enhanced properties include but are not limited to longer terminal half-life, larger area under the curve, increased time in which the blood concentration remains within the therapeutic window, increased time between consecutive doses results in blood concentrations within the therapeutic window, and decreased dose in IU over time that can be administered compared to a FVIII not linked to XTEN, yet still result in a blood concentration above a threshold concentration needed for a procoagulant effect. In some embodiments, the terminal half-life of the fusion proteins of the embodiments, including but not limited to those configured according to formulae I-VIII, administered to a subject is increased at least about three-fold, or at least about four-fold, or at least about five-fold, or at least about six-fold, or at least about eight-fold, or at least about ten-fold, or at least about 20-fold, or at least about 40-fold, or at least about 60-fold or even higher as compared to FVIII not linked to XTEN and administered to a subject at a comparable dose. In other embodiments, the terminal half-life of the fusion protein and in one of the configurations of formulae I-VIII administered to a subject is at least about 12 h, or at least about 24 h, or at least about 48 h, or at least about 72 h, or at least about 96 h, or at least about 120 h, or at least about 144 h, or at least about 21 days or greater. In other embodiments, the enhanced pharmacokinetic property of the fusion proteins of the embodiments is the property of maintaining a circulating blood concentration of procoagulant fusion protein comprising FVIII to a subject in need thereof above a threshold concentration of 0.01 IU/ml, or 0.05 IU/ml, or 0.1 IU/ml, or 0.2 IU/ml, or 0.3 IU/ml, or 0.4 IU/ml or 0.5 IU/ml for a period that is at least about two fold, or at least about three-fold, or at least about four-fold, or at least about five-fold, or at least about six-fold, or at least about eight-fold, or at least about ten-fold, or at least about 20-fold, or at least about 40-fold, or at least about 60-fold longer compared to the corresponding FVIII not linked to XTEN and administered to a subject at a comparable dose. The increase in half-life and time spent above the threshold concentration permits less frequent dosing and decreased amounts of the fusion protein (in moles equivalent) that are administered to a subject, compared to the corresponding FVIII not linked to XTEN. In one embodiment, administration of a subject fusion protein to a subject using a therapeutically-effective dose regimen results in a gain in time of at least two-fold, or at least three-fold, or at least four-fold, or at least five-fold, or at least six-fold, or at least eight-fold, or at least 10-fold, or at least about 20-fold, or at least about 40-fold, or at least about 60-fold or higher between at least two consecutive Cₘₐₓ peaks and/or Cₘᵢₙ troughs for blood levels of the fusion protein compared to the corresponding FVIII not linked to the XTEN and administered using a comparable dose regimen to a subject.

In some embodiments, the XTEN enhances the thermostability of FVIII when linked to the XTEN wherein the thermostability is ascertained by measuring the retention of biological activity after exposure to a temperature of about 37° C. for at least about 7 days of the biologically active protein in comparison to the biologically active protein not linked to the XTEN. In one embodiment of the foregoing, the retention of biological activity increases by at least about 50%, at least about 60%, at least about 70%, at least about 80%, at least about 90%, at least about 100%, or about 150%, at least about 200%, at least about 300%, or about 500% longer compared to the CF not linked to the XTEN.

In some embodiments, the subject compositions are configured to have reduced binding affinity for a clearance receptor in a subject as compared to the corresponding FVIII not linked to the XTEN. In one embodiment, the CFXTEN fusion protein exhibits binding affinity for a clearance receptor of the FVIII in the range of about 0.01%-30%, or about 0.1% to about 20%, or about 1% to about 15%, or about 2% to about 10% of the binding affinity of the corresponding FVIII not linked to the XTEN. In another embodiment, a fusion protein with reduced affinity for a clearance receptor has reduced active clearance and a corresponding increase in half-life of at least about 2-fold, or 3-fold, or at least 4-fold, or at least about 5-fold, or at least about 6-fold, or at least about 7-fold, or at least about 8-fold, or at least about 9-fold, or at least about 10-fold, or at least about 12-fold, or at least about 15-fold, or at least about 17-fold, or at least about 20-fold longer compared to the corresponding FVIII that is not linked to the XTEN.

In an embodiment, the invention provides an isolated fusion protein comprising FVIII and one or more XTEN wherein the fusion protein exhibits increased solubility of at least three-fold, or at least about four-fold, or at least about five-fold, or at least about six-fold, or at least about seven-fold, or at least about eight-fold, or at least about nine-fold, or at least about ten-fold, or at least about 15-fold, or at least a 20-fold, or at least 40-fold, or at least 60-fold at physiologic conditions compared to the FVIII not linked to XTEN.

The following are non-limiting embodiments of the invention:

Item 1. An isolated fusion protein comprising at least one extended recombinant polypeptide (XTEN), wherein said fusion protein having a structure of formula VIII:

(XTEN)u-(S)a-(A1)-(S)b-(XTEN)v-(S)b-(A2)-(B1)-(S)c-(XTEN)w-(S)c-(B2)-(A3)-(S)d-(XTEN)x-(S)d-(C1)-(S)e-(XTEN)y-(S)e-(C2)-(S)f-(XTEN)z VIII

wherein independently for each occurrence,

a) A1 is an A1 domain of FVIII;

b) A2 is an A2 domain of FVIII;

c) B1 is a fragment of the N-terminal end of the B domain having amino acid residues from residue number 740 to about number 745 of a native FVIII sequence;

d) B2 is a fragment of the C-terminal end of the B domain having amino acid residues from about residue numbers 1640 to number 1689 of a native FVIII sequence;

e) A3 is an A3 domain of FVIII;

f) C1 is a C1 domain of FVIII;

g) C2 is a C2 domain of FVIII;

h) S is a spacer sequence having between 1 to about 50 amino acid residues that can optionally include a cleavage sequence or amino acids compatible with restrictions sites, wherein for each occurrence, if there is any, the sequence of the spacer can be the same or different;

i) a is either 0 or 1;

j) b is either 0 or 1;

k) c is either 0 or 1;

l) d is either 0 or 1;

m) e is either 0 or 1;

n) f is either 0 or 1;

o) u is either 0 or 1;

p) v is either 0 or 1;

q) w is 0 or 1;

r) x is either 0 or 1;

s) y is either 0 or 1;

t) z is either 0 or 1, with the proviso that u+v+w+x+y+z>1; and

wherein the at least one XTEN is characterized in that:

a. the XTEN comprises at least 36 amino acid residues;

b. the sum of glycine (G), alanine (A), serine (S), threonine (T), glutamate (E) and proline (P) residues constitutes more than about 80% of the total amino acid residues of the XTEN;

c. the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

d. the XTEN has greater than 90% random coil formation as determined by GOR algorithm;

e. the XTEN has less than 2% alpha helices and 2% beta-sheets as determined by Chou-Fasman algorithm;

f. the XTEN lacks a predicted T-cell epitope when analyzed by TEPITOPE algorithm, wherein the TEPITOPE threshold score for said prediction by said algorithm has a threshold of −9.

Item 2. The isolated fusion protein of item 1, comprising at least two XTENs, wherein the cumulative length of the XTENs is between about 100 to about 3000 amino acid residues.
Item 3. The isolated fusion protein of item 2, wherein each XTEN exhibits at least 90% sequence identity to a sequence of comparable length from any one of Table 4, Table 9, Table 10, Table 11, Table 12, and Table 13, when optimally aligned.
Item 4. The isolated fusion protein of any one of items 1-3, wherein the optional cleavage sequence(s) are cleavable by a mammalian protease selected from the group consisting of factor XIa, factor XIIa, kallikrein, factor VIIa, factor IXa, factor Xa, factor IIa (thrombin), Elastase-2, MMP-12, MMP13, MMP-17 and MMP-20, wherein upon cleavage of the cleavage sequences, at least one XTEN is cleaved from the fusion protein and the cleaved fusion protein exhibits an increase in procoagulant activity of at least about 30% compared to the uncleaved fusion protein.
Item 5. The isolated fusion protein of any one of items 1-4, wherein said fusion protein exhibits a prolonged in vitro half-life as compared to a corresponding factor VIII polypeptide lacking said XTEN.
Item 6. The isolated fusion protein of any one of items 1-5, wherein said fusion protein exhibits a terminal half-life longer than at least 48 hours when administered to a subject.
Item 7. An isolated fusion protein comprising a factor VIII polypeptide and at least one extended recombinant polypeptide (XTEN), wherein said factor VIII polypeptide comprises A1 domain, A2 domain, A3 domain, C1 domain, C2 domain and optionally all or a portion of B domain, and wherein said at least one XTEN is linked to said factor VIII polypeptide at (i) the C-terminus of said factor VIII polypeptide; (ii) within B domain of said factor VIII polypeptide if all or a portion of B domain is present; (iii) within the A1 domain of said factor VIII polypeptide; (iv) within the A2 domain of said factor VIII polypeptide; (v) within the A3 domain of said factor VIII polypeptide; (vi) within the C1 domain of said factor VIII polypeptide; or (vii) within the C2 domain of said factor VIII polypeptide; and wherein the XTEN is characterized in that:

a. the XTEN comprises at least 36 amino acid residues;

b. the sum of glycine (G), alanine (A), serine (S), threonine (T), glutamate (E) and proline (P) residues constitutes more than about 80% of the total amino acid residues of the XTEN;

c. the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

d. the XTEN has greater than 90% random coil formation as determined by GOR algorithm;

e. the XTEN has less than 2% alpha helices and 2% beta-sheets as determined by Chou-Fasman algorithm;

f. the XTEN lacks a predicted T-cell epitope when analyzed by TEPITOPE algorithm, wherein the TEPITOPE threshold score for said prediction by said algorithm has a threshold of −9, and wherein said fusion protein exhibits a terminal half-life that is longer than about 48 hours when administered to a subject.

Item 8. The isolated fusion protein of item 7 comprising at least another XTEN linked to said factor VIII polypeptide at the C-terminus of said factor VIII polypeptide, and within the B domain of said factor VIII polypeptide.
Item 9. The isolated fusion protein of item 7 comprising a first XTEN sequence linked to said factor VIII polypeptide at the C-terminus of said factor VIII polypeptide, and at least a second XTEN within the B domain of said factor VIII polypeptide, wherein the second XTEN is linked to the C-terminal end of about amino acid residue number 740 to about 750 and to the N-terminal end of amino acid residue numbers 1640 to about 1689 of a native FVIII sequence, wherein the cumulative length of the XTEN is at least about 100 amino acid residues.
Item 10. The isolated fusion protein of item 7 comprising at least one XTEN sequence located within B domain of said factor VIII polypeptide.
Item 11. The isolated fusion protein of item 7 comprising at least a second XTEN, wherein said at least second XTEN is linked to said factor VIII polypeptide at one or more locations selected from:

a. an insertion location from Table 5;

b. a location between any two adjacent domains of said factor VIII polypeptide, wherein said two adjacent domains are selected from the group consisting of A1 and A2 domains, A2 and B domains, B and A3 domains, A3 and C1 domains, and C1 and C2 domains;

c. the N-terminus of said factor VIII polypeptide; and

d. the C-terminus of said factor VIII polypeptide,

Item 12. The isolated fusion protein of any one of items 8-11, the second XTEN having a sequence characterized in that:

a) the XTEN comprises at least 36 amino acid residues;

b) the sum of glycine (G), alanine (A), serine (S), threonine (T), glutamate (E) and proline (P) residues constitutes more than about 80% of the total amino acid residues of the XTEN;

c) the XTEN sequence is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues does not occur more than twice in each of the sequence motifs; or (iii) the XTEN sequence has a subsequence score of less than 10;

d) the XTEN has greater than 90% random coil formation as determined by GOR algorithm;

e) the XTEN has less than 2% alpha helices and 2% beta-sheets as determined by Chou-Fasman algorithm; and

f) the XTEN lacks a predicted T-cell epitope when analyzed by TEPITOPE algorithm, wherein the TEPITOPE threshold score for said prediction by said algorithm has a threshold of −9.

Item 13. The isolated fusion protein of any one of preceding items, wherein the factor VIII polypeptide has at least 90% sequence identity compared to a sequence selected from Table 1, when optimally aligned.
Item 14. The isolated fusion protein of any one of preceding items, wherein the factor VIII polypeptide comprises human factor VIII.
Item 15. The isolated fusion protein of any one of preceding items, wherein the factor VIII polypeptide comprises a B-domain deleted variant of human factor VIII.
Item 16. The isolated fusion protein of item 11, wherein the XTEN is linked to the C-terminus of the factor VIII polypeptide.
Item 17. The isolated fusion protein of item 11, wherein the XTEN is linked to the N-terminus of the factor VIII polypeptide.
Item 18. The isolated fusion protein of any one of the preceding items, wherein the fusion protein exhibits an apparent molecular weight factor of at least about 2.
Item 19. The isolated fusion protein of any one of items 7-18, wherein the XTEN has at least 90% sequence identity compared to a sequence of comparable length selected from any one of Table 4, Table 9, Table 10, Table 11, Table 12, and Table 13, when optimally aligned.
Item 20. The isolated fusion protein of any one of items 7-18, wherein the factor VIII polypeptide is linked to the XTEN via one or two cleavage sequences that each is cleavable by a mammalian protease selected from the group consisting of factor XIa, factor XIIa, kallikrein, factor VIIa, factor IXa, factor Xa, factor IIa (thrombin), Elastase-2, MMP-12, MMP13, MMP-17 and MMP-20, wherein cleavage at the cleavage sequence by the mammalian protease releases the factor VIII sequence from the XTEN sequence, and wherein the released factor VIII sequence exhibits an increase in procoagulant activity of at least about 30% compared to the uncleaved fusion protein.
Item 21. The isolated fusion protein of item 20, wherein the cleavage sequence(s) are cleavable by factor XIa.
Item 22. The isolated fusion protein any one of items 7-21, comprising multiple XTENs located at different locations of the factor VIII polypeptide, wherein said different locations are selected from:

a. an insertion location from Table 5;

b. a location between any two adjacent domains in the factor VIII sequence, wherein said two adjacent domains are selected from the group consisting of A1 and A2, A2 and B, B and A3, A3 and C1, and C1 and C2;

c. the N-terminus of the factor VIII sequence; and

d. the C-terminus of the factor VIII sequence;

wherein the cumulative length of the multiple XTENs is at least about 100 to about 3000 amino acid residues.
Item 23. The isolated fusion protein of any one of items 7-22, wherein said fusion protein exhibits a prolonged in vitro half-life as compared to a corresponding factor VIII polypeptide lacking said XTEN.
Item 24. The isolated fusion protein of any one of items 7-23, wherein said fusion protein exhibits a terminal half-life longer than at least 48 hours when administered to a subject.
Item 25. A pharmaceutical composition comprising the fusion protein of any one of the preceding items and a pharmaceutically acceptable carrier.
Item 26. A method of treating a coagulopathy in a subject, comprising administering to said subject a composition comprising a therapeutically effective amount of the pharmaceutical composition of item 25.
Item 27. The method of item 26, wherein after said administration, a concentration of procoagulant factor VIII is maintained at about 0.05 IU/ml or more for at least 48 hours after said administration.
Item 28. The method of item 26, wherein said coagulopathy is hemophilia A.
Item 29. A method of treating a bleeding episode in a subject, comprising administering to said subject a composition comprising a therapeutically effective amount of the pharmaceutical composition of item 25, wherein the therapeutically effective amount of the fusion protein arrests a bleeding episode for a period that is at least three-fold longer compared to the corresponding factor VIII polypeptide lacking said at least one XTEN when said corresponding factor VIII is administered to a subject at a comparable dose.
Item 30. A fusion protein used in the treatment of hemophilia A, comprising the fusion protein of any one of items 1-24.
Item 31. An isolated fusion protein comprising a polypeptide having at least 90% sequence identity compared to a sequence of comparable length selected from any one of Table 14, Table 28, Table 29 and Table 30.
Item 32. An isolated fusion protein comprising a factor VIII polypeptide and at least one extended recombinant polypeptide (XTEN), wherein said factor VIII polypeptide comprises A1 domain, A2 domain, A3 domain, and C1 domain, and wherein said at least one XTEN is linked to said factor VIII polypeptide at one or more insertion locations selected from the group consisting of:

a. the C-terminus of said factor VIII polypeptide;

b. within the A1 domain of said factor VIII polypeptide;

c. within the A2 domain of said factor VIII polypeptide;

d. within the A3 domain of said factor VIII polypeptide;

e. within the C1 domain of said factor VIII polypeptide;

f. one or more location between any two adjacent domains of said factor VIII polypeptide,

g. the N-terminus of said factor VIII polypeptide;

h. one or more location from FIG. 5;

i. one or more insertion location from Table 5; and

wherein the at least one XTEN is characterized in that:

i. the XTEN comprises at least 36 amino acid residues;

ii. the sum of glycine (G), alanine (A), serine (S), threonine (T), glutamate (E) and proline (P) residues constitutes more than about 80% of the total amino acid residues of the XTEN;

iii. the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

iv. the XTEN has greater than 90% random coil formation as determined by GOR algorithm;

v. the XTEN has less than 2% alpha helices and 2% beta-sheets as determined by Chou-Fasman algorithm; and

vi. the XTEN lacks a predicted T-cell epitope when analyzed by TEPITOPE algorithm, wherein the TEPITOPE threshold score for said prediction by said algorithm has a threshold of −9.

Item 33. An isolated fusion protein comprising a factor VIII polypeptide and at least one extended recombinant polypeptide (XTEN), wherein said factor VIII polypeptide comprises A1 domain, A2 domain, A3 domain, and C1 domain, and wherein said at least one XTEN is linked to said factor VIII polypeptide at one or more insertion locations from table 25 and is characterized in that:

i. the XTEN comprises at least 36 amino acid residues;

ii. the sum of glycine (G), alanine (A), serine (S), threonine (T), glutamate (E) and proline (P) residues constitutes more than about 80% of the total amino acid residues of the XTEN;

iii. the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

iv. the XTEN has greater than 90% random coil formation as determined by GOR algorithm;

v. the XTEN has less than 2% alpha helices and 2% beta-sheets as determined by Chou-Fasman algorithm; and

vi. the XTEN lacks a predicted T-cell epitope when analyzed by TEPITOPE algorithm, wherein the TEPITOPE threshold score for said prediction by said algorithm has a threshold of −9.

Item 34. The fusion protein of item 32 or 33, wherein said two adjacent domains are selected from the group consisting of the A1 and A2 domains, the A2 and A3 domains, and the A3 and C1 domains.
Item 35. The fusion protein of any one of items 32 to 34, wherein said factor VIII polypeptide further comprises C2 domain.
Item 36. The fusion protein of item 35, wherein at least one XTEN is inserted within the C2 domain, N-terminus of the C2 domain, C-terminus of the C2 domain, or a combination thereof.
Item 37. The fusion protein of any one of items 32 to 36, wherein said Factor VIII comprises a full-length B domain or a partially deleted B domain.
Item 38. The fusion protein of item 37, wherein at least one XTEN is inserted within the full-length B domain or partially deleted B domain, N-terminus of the full-length B domain or partially deleted B domain, C-terminus of the full-length B domain or partially deleted B domain, or a combination thereof.
Item 39. The fusion protein of any one of items 32 to 38, wherein said A3 domain comprises an a3 acidic region or a portion thereof.
Item 40. The fusion protein of item 27, wherein at least one XTEN is inserted within the a3 acidic region or the portion thereof, N-terminus of the a3 acidic region or the portion thereof, C-terminus of the a3 acidic region or the portion thereof, or a combination thereof.
Item 41. The fusion protein of any one of items 32 to 40, further comprising one or more spacer linked to said at least one XTEN.
Item 42. The fusion protein of item 41, wherein said spacer comprises about 1 to about 50 amino acid residues that optionally includes a cleavage sequence or amino acids compatible with restriction sites, wherein for each occurrence, if there is any, the sequence of the spacer is the same or different.
Item 43. An isolated fusion protein comprising a structure of formula (A):

(XTEN)v-(S)a-(A1)-(S)b-(XTEN)w-(S)b-(A2)-(S)c-(XTEN)x-(S)c-(A3)-(S)d-(XTEN)y-(S)d-(C1)-(S)e-(XTEN)z (A)

wherein independently for each occurrence,

u) A1 is an A1 domain of FVIII;

v) A2 is an A2 domain of FVIII;

w) A3 is an A3 domain of FVIII;

x) C1 is a C1 domain of FVIII;

y) S is a spacer sequence having between 1 to about 50 amino acid residues that optionally includes a cleavage sequence or amino acids compatible with restrictions sites, wherein for each occurrence, if there is any, the sequence of the spacer is the same or different;

wherein

(i) a is either 0 or 1;

(ii) b is either 0 or 1;

(iii) c is either 0 or 1;

(iv) d is either 0 or 1;

(v) e is either 0 or 1;

(vi) v is either 0 or 1;

(vii) w is 0 or 1;

(viii) x is either 0 or 1;

(ix) y is either 0 or 1;

(x) z is either 0 or 1,

with the proviso that v+w+x+y+z>1,
wherein said XTEN is characterized in that:

(1). the XTEN comprises at least 36 amino acid residues;

(2). the sum of glycine (G), alanine (A), serine (S), threonine (T), glutamate (E) and proline (P) residues constitutes more than about 80% of the total amino acid residues of the XTEN;

(3). the XTEN is substantially non-repetitive such that (1) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

(4). the XTEN has greater than 90% random coil formation as determined by GOR algorithm;

(5). the XTEN has less than 2% alpha helices and 2% beta-sheets as determined by Chou-Fasman algorithm; and

(6). the XTEN lacks a predicted T-cell epitope when analyzed by TEPITOPE algorithm, wherein the TEPITOPE threshold score for said prediction by said algorithm has a threshold of −9.

Item 44. The fusion protein of item 43, wherein said factor VIII polypeptide further comprises C2 domain.
Item 45. The fusion protein of item 44, wherein at least one XTEN is inserted within the C2 domain, N-terminus of the C2 domain, C-terminus of the C2 domain, or a combination thereof.
Item 46. The fusion protein of any one of items 43 to 45, wherein said Factor VIII comprises a full or a partially deleted B domain anywhere between the A2 and the A3.
Item 47. The fusion protein of item 46, wherein at least one XTEN is inserted within the full-length B domain or partially deleted B domain, N-terminus of the full-length B domain or partially deleted B domain, C-terminus of the full-length B domain or partially deleted B domain, or a combination thereof.
Item 48. The fusion protein of any one of items 43 to 47, wherein said A3 domain comprises an a3 acidic region or a portion thereof.
Item 49. The fusion protein of item 48, wherein at least one XTEN is inserted within the a3 acidic region or the portion thereof, N-terminus of the a3 acidic region or the portion thereof, C-terminus of the a3 acidic region or the portion thereof, or a combination thereof.
Item 50. The fusion protein of item 44, wherein at least one XTEN is further inserted within the A1, the A2, the A3, the C1, the C2, or a combination of two or more thereof.
Item 51. The fusion protein of any one of items 37-38 and 46-47, wherein said B domain comprises amino acid residues 741 to 743 of mature FVIII and/or amino acid residues 1638 to 1648 of mature FVIII.
Item 52. The fusion protein of any one of items 32 to 51, wherein said at least one XTEN is inserted right after Arginine at residue 1648 of mature FVIII.
Item 53. The fusion protein of any one of items 32 to 52, wherein said at least one XTEN is inserted in one or more thrombin cleavage site selected from the group consisting of amino acid residues 372 of FVIII, 740 of FVIII, and 1689 of FVIII.
Item 54. The fusion protein of any one of items 43 to 53, wherein the sum of v, w, x, y, and z, equals to 2, 3, 4, 5, 6, 7, 8, 9, or 10.
Item 55. The fusion protein of any one of items 32 to 54, wherein said factor VIII polypeptide comprises a heavy chain and a light chain, wherein said heavy chain comprises the A1 domain and the A2 domain, and said light chain comprises the A3 domain and the C1 domain.
Item 56. The fusion protein of item 55, wherein said heavy chain further comprises a partially deleted B domain and/or the light chain further comprises a partially deleted B domain.
Item 57. The fusion protein of any one of items 42-56, wherein the optional cleavage sequence(s) are cleavable by a mammalian protease selected from the group consisting of factor XIa, factor XIIa, kallikrein, factor VIIa, factor IXa, factor Xa, factor IIa (thrombin), Elastase-2, MMP-12, MMP13, MMP-17 and MMP-20, wherein upon cleavage of the cleavage sequences, at least one XTEN is cleaved from the fusion protein and the cleaved fusion protein exhibits an increase in procoagulant activity of at least about 30% compared to the uncleaved fusion protein.
Item 58. The fusion protein of any one of items 32 to 57, wherein one or more of said at least one XTEN is 36 amino acids, 42 amino acids, 144 amino acids, 288 amino acids, 576 amino acids, or 864 amino acids in length.
Item 59. The fusion protein of any one of items 32 to 57, wherein one or more of said at least one XTEN is selected from the group consisting of: XTEN_AE42, XTEN_AE864, XTEN_AE576, XTEN_AE288, XTEN_AE144, XTEN_AG864, XTEN_AG576, XTEN_AG288, and XTEN_AG144
Item 60. The fusion protein of any one of items 32 to 59, which comprises at least two XTENs, wherein the cumulative length of the XTENs is between about 100 to about 3000 amino acid residues.
Item 61. The fusion protein of any one of items 32 to 60, wherein said fusion protein exhibits a prolonged in vitro half-life as compared to a corresponding factor VIII polypeptide lacking said XTEN.
Item 62. The fusion protein of any one of items 32-61, wherein said fusion protein exhibits a terminal half-life longer than at least 48 hours when administered to a subject.
Item 63. The fusion protein of any one of items 32 to 62, wherein a first XTEN of said at least one XTEN is linked to said factor VIII polypeptide at the C-terminus of said factor VIII polypeptide, and a second XTEN of said at least one XTEN is linked within the B domain of said factor VIII polypeptide.
Item 64. The fusion protein of item 63, wherein said second XTEN is linked between amino acid residue 743 and amino acid residue 1638 of mature FVIII.
Item 65. The fusion protein of item 63 or 64, wherein said first XTEN or said second XTEN has 36 amino acids, 42 amino acids, 144 amino acids, 288 amino acids, 576 amino acids, or 864 amino acids in length.
Item 66. The fusion protein of any one of items 63 to 65, wherein said first XTEN or said second XTEN is selected from the group consisting of: XTEN_AE42_{—}4, XTEN_AE864, XTEN_AE576, XTEN_AE288, XTEN_AE144, XTEN_AG864, XTEN_AG576, XTEN_AG288, and XTEN_AG144.
Item 67. The fusion protein of any one of the preceding items, wherein the cumulative length of the XTENs is at least about 100 amino acid residues.
Item 68. The fusion protein of any one of items 32 to 67, further comprising one or more XTEN linked to the factor VIII polypeptide at one or more locations selected from the group consisting of:

a. one or more insertion location from Table 5 or Table 25;

b. one or more insertion location from FIG. 5;

c. within the B domain of said factor VIII polypeptide;

d. within the A1 domain of said factor VIII polypeptide;

e. within the A2 domain of said factor VIII polypeptide;

f. within the a3 acidic region of said factor VIII polypeptide;

g. within the A3 domain of said factor VIII polypeptide;

h. within the C1 domain of said factor VIII polypeptide;

i. within the C2 domain of said factor VIII polypeptide;

j. one or more insertion location between any two adjacent domains of said factor VIII polypeptide, wherein said two adjacent domains are selected from the group consisting of A1 and A2 domains, A2 and B domains, B domain and a3 region, A2 domain and a3 region when B domain is completely deleted, a3 region and A3 domains, A3 and C1 domains, and C1 and C2 domains;

k. the N-terminus of said factor VIII polypeptide; and

l. the C-terminus of said factor VIII polypeptide.

Item 69. The fusion protein of any one of items 32 to 67, further comprising one or more XTEN linked to the factor VIII polypeptide at one or more locations from Table 25.
Item 70. The fusion protein item 68 or 69, wherein the one or more XTEN is characterized in that:

a. the XTEN comprises at least 36 amino acid residues;

b. the sum of glycine (G), alanine (A), serine (S), threonine (T), glutamate (E) and proline (P) residues constitutes more than about 80% of the total amino acid residues of the XTEN;

c. the XTEN sequence is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues does not occur more than twice in each of the sequence motifs; or (iii) the XTEN sequence has a subsequence score of less than 10;

d. the XTEN has greater than 90% random coil formation as determined by GOR algorithm;

e. the XTEN has less than 2% alpha helices and 2% beta-sheets as determined by Chou-Fasman algorithm; and

f. the XTEN lacks a predicted T-cell epitope when analyzed by TEPITOPE algorithm, wherein the TEPITOPE threshold score for said prediction by said algorithm has a threshold of −9.

Item 71. The fusion protein of any one of items 68 to 70, wherein said one or more XTEN has 36 amino acids, 42 amino acids, 144 amino acids, 288 amino acids, 576 amino acids, or 864 amino acids in length.
Item 72. The fusion protein of any one of items 68 to 70, wherein said one or more XTEN is selected from the group consisting of: XTEN_AE42_{—}4, XTEN_AE864, XTEN_AE576, XTEN_AE288, XTEN_AE144, XTEN_AG864, XTEN_AG576, XTEN_AG288, and XTEN_AG144.
Item 73. The fusion protein of any one of the preceding items, wherein the factor VIII polypeptide has at least 90% sequence identity compared to a sequence selected from Table 1 or Table 31, when optimally aligned.
Item 74. The fusion protein of any one of the preceding items, wherein the factor VIII polypeptide comprises human factor VIII.
Item 75. The fusion protein of any one of the preceding items, wherein said at least one XTEN is linked to the C-terminus of the factor VIII polypeptide.
Item 76. The fusion protein of the any one of the preceding item, wherein said at least one XTEN is linked to the N-terminus of the factor VIII polypeptide.
Item 77. The fusion protein of the any one of the preceding items, wherein said at least one XTEN is linked to an insertion location from Table 25.
Item 78. The fusion protein of any one of the preceding items, wherein the fusion protein exhibits an apparent molecular weight factor of at least about 2.
Item 79. The fusion protein of any one of items the preceding items, wherein the XTEN has at least 90% sequence identity compared to a sequence of comparable length selected from any one of Table 4, Table 9, Table 10, Table 11, Table 12, and Table 13, when optimally aligned.
Item 80. The fusion protein of item 57, wherein the cleavage sequence(s) are cleavable by factor XIa.
Item 81. A pharmaceutical composition comprising the fusion protein of any one of the preceding items and a pharmaceutically acceptable carrier.
Item 82. A method of treating a coagulopathy in a subject, comprising administering to said subject a composition comprising a therapeutically effective amount of the pharmaceutical composition of item 81.
Item 83. The method of item 82, wherein after said administration, a concentration of procoagulant factor VIII is maintained at about 0.05 IU/ml or more for at least 48 hours after said administration.
Item 84. The method of item 82 or 83, wherein said coagulopathy is hemophilia A.
Item 85. A method of treating a bleeding episode in a subject, comprising administering to said subject a composition comprising a therapeutically effective amount of the pharmaceutical composition of item 82, wherein the therapeutically effective amount of the fusion protein arrests a bleeding episode for a period that is at least three-fold longer compared to the corresponding factor VIII polypeptide lacking said at least one XTEN when said corresponding factor VIII is administered to a subject at a comparable dose.
Item 86. A fusion protein used in the treatment of hemophilia A, comprising the fusion protein of any one of items 1-85.

In some embodiments, the subject compositions exhibit enhanced pharmacokinetic properties characterized in that: (i) they have a longer half-life when administered to a subject compared to the corresponding FVIII coagulation factor not linked to the XTEN administered to a subject under an otherwise equivalent dose; (ii) when a smaller IU amount of the fusion protein is administered to a subject in comparison to the corresponding coagulation factor VIII that lacks the XTEN administered to a subject under an otherwise equivalent dose regimen, the fusion protein achieves a comparable area under the curve (AUC) as the corresponding FVIII not linked to the XTEN; (iii) when a smaller IU amount of the fusion protein is administered to a subject in comparison to the corresponding FVIII that lacks the XTEN administered to a subject under an otherwise equivalent dose regimen, the fusion protein achieves a comparable therapeutic effect as the corresponding coagulation factor VIII not linked to the XTEN; (iv) when the fusion protein is administered to a subject less frequently in comparison to the corresponding coagulation factor VIII not linked to the XTEN administered to a subject using an otherwise equivalent IU dose, the fusion protein achieves a comparable area under the curve (AUC) as the corresponding coagulation factor VIII not linked to the XTEN; (v) when the fusion protein is administered to a subject less frequently in comparison to the corresponding coagulation factor VIII not linked to the XTEN administered to a subject using an otherwise equivalent IU dose, the fusion protein achieves a comparable therapeutic effect as the corresponding coagulation factor VIII not linked to the XTEN; (vi) when an accumulatively smaller IU amount of the fusion protein is administered to a subject in comparison to the corresponding coagulation factor not linked to the XTEN administered to a subject under an otherwise equivalent dose period, the fusion protein achieves comparable area under the curve (AUC) as the corresponding coagulation factor FVIII not linked to the XTEN; or (vii) when an accumulatively smaller IU amount of the fusion protein is administered to a subject in comparison to the corresponding coagulation factor VIII not linked to the XTEN administered to a subject under an otherwise equivalent dose period, the fusion protein achieves comparable therapeutic effect as the corresponding coagulation factor not linked to the XTEN. The accumulative smaller IU amount is measured for a period of at least about one week, or about 14 days, or about 21 days, or about one month.

The present invention provides a method of producing a fusion protein comprising a factor VIII polypeptide fused to one or more extended recombinant polypeptides (XTEN), comprising: (a) providing a host cell comprising a recombinant polynucleotide molecule encoding the fusion protein; (b) culturing the host cell under conditions permitting the expression of the fusion protein; and (c) recovering the fusion protein from the culture. In one embodiment of the method, the factor VIII of the fusion protein has at least about 80%, or at least about 90%, or at least about 95%, or at least about 99% sequence identity compared to a sequence selected from Table 1 or Table 31 and the one or more XTEN of the expressed fusion protein has at least about 80%, or about 90%, or about 91%, or about 92%, or about 93%, or about 94%, or about 95%, or about 96%, or about 97%, or about 98%, or about 99% to about 100% sequence identity compared to a sequence selected from Table 4 or Table 8 or Table 9 or Table 10 or Table 11 or Table 12. In one embodiment of the method, the host cell is a eukaryotic cell selected from CHO cell, BHK, HEK, COS, HEK-293 or COS-7. In another embodiment of the method, the isolated fusion protein is recovered from the host cell cytoplasm in substantially soluble form.

The present invention provides isolated nucleic acids comprising a polynucleotide sequence selected from (a) a polynucleotide encoding the fusion protein of any of the foregoing embodiments, or (b) the complement of the polynucleotide of (a). In one embodiment, the invention provides an isolated nucleic acid comprising (a) a polynucleotide sequence encoding a polypeptide sequence that has at least 80% sequence identity, or about 85%, or at least about 90%, or about 91%, or about 92%, or about 93%, or about 94%, or about 95%, or about 96%, or about 97%, or about 98%, or about 99% to about 100% sequence identity to a polypeptide of any one of Tables 14 and 28-30; or (b) the complement of the polynucleotide of (a). The invention provides expression vectors comprising the nucleic acid of any of the embodiments hereinabove described in this paragraph. In one embodiment, the expression vector of the foregoing further comprises a recombinant regulatory sequence operably linked to the polynucleotide sequence. In another embodiment, the polynucleotide sequence of the expression vectors of the foregoing is fused in frame to a polynucleotide encoding a secretion signal sequence, which can be a factor VIII native signal sequence. The invention provides a host cell that comprises an expression vector of any of the embodiments hereinabove described in this paragraph. In one embodiment, the host cell is a eukaryotic cell. In another embodiment, the host cell is a CHO cell. In another embodiment, the host cell is an HEK cell. In another embodiment, the host cell is a BHK cell. In another embodiment, the host cell is a COS-7 cell. In another embodiment, the host cell is a HEK293 cell.

Additionally, the present invention provides pharmaceutical compositions comprising the fusion protein of any of the foregoing embodiments described herein and a pharmaceutically acceptable carrier. The pharmaceutical composition can be administered by any suitable means, including parenterally, subcutaneously, intramuscularly, or intravenously. The invention further provides a method of treating a coagulopathy or a factor VIII-related disease, disorder or condition in a subject, comprising administering to the subject a therapeutically effective amount of the foregoing pharmaceutical composition wherein the administration resulted in an improvement of at least one parameter associated with a FVIII disease, disorder or condition wherein the improvement is greater or of longer duration than that obtained by administration of FVIII not linked to XTEN and administered at a comparable dose. Non-limiting examples of parameters include blood concentrations of FVIII, activated partial prothrombin (aPTT) assay time, one-stage or two-stage clotting assay time, delayed onset of a bleeding episode, chromogenic FVIII assay time, bleeding times, or thrombelastography (TEG or ROTEM) assays, among others known in the art. The factor VIII-related disease, disorder or condition includes hemophilia A, bleeding disorders (e.g., defective platelet function, thrombocytopenia or von Willebrand's disease), vascular injury, bleeding from trauma or surgery, bleeding due to anticoagulant therapy, bleeding due to liver disease, circulating antibodies to FVIII, and defects in factor VIII. In a preferred embodiment of the method of treatment, the coagulopathy is hemophilia A. In an embodiment of the method of treatment, the pharmaceutical compositions is administered to a subject in need thereof in an amount sufficient to control a bleeding episode. In another embodiment of the method of treatment, the pharmaceutical composition is administered to a subject in need thereof in an amount sufficient to increase the circulating FVIII procoagulant concentration to a threshold concentration greater than 0.01 IU/ml (1% of normal), or greater than 0.01-0.05 IU/ml (1%-5% of normal), or greater than 0.05 to about 0.40 IU/ml (>5%-<40% of normal). In the foregoing embodiment, the concentration is maintained at or above the threshold concentration for at least about 12 h, or at least about 24 h, or at least about 48 h, or at least about 72 h, or at least about 96 h, or at least about 120 h, or at least about 144 h, or at least about 168 h, or greater. In another embodiment of the method of treatment, the pharmaceutical compositions is administered to a subject with anti-FVIII antibodies. In one embodiment, wherein the pharmaceutical composition is administered at a therapeutically effective amount, the administration results in a gain in time spent before onset of a bleeding episode of at least two-fold longer than the corresponding FVIII not linked to the XTEN, or alternatively, at least three-fold, at least four-fold, or five-fold, or six-fold, or seven-fold, or eight-fold, or nine-fold, or at least 10-fold, or at least 20-fold longer than the corresponding FVIII not linked to XTEN and administered at a comparable dose to a subject. In another embodiment, the invention provides a method of treatment wherein the administration of a therapeutically effective amount of the pharmaceutical composition arrests a bleeding episode for a period that is at least two-fold longer, or at least three-fold longer, or at least four-fold longer, or at least five-fold longer compared to a composition comprising the corresponding factor VIII polypeptide lacking said at least one XTEN when said corresponding factor VIII composition is administered to a subject at a comparable dose.

In another embodiment, the present invention provides a method of treating a factor VIII-related disease, disorder or condition, comprising administering the pharmaceutical composition described above to a subject using multiple consecutive doses of the pharmaceutical composition administered using a therapeutically effective dose regimen wherein the administration results in the improvement of at least one parameter wherein the improvement is greater or of longer duration than that obtained by administration of FVIII not linked to XTEN and administered under a therapeutically effective dose regimen. In one embodiment of the foregoing, the therapeutically effective dose regimen can result in a gain in time of at least three-fold, or alternatively, at least four-fold, or five-fold, or six-fold, or seven-fold, or eight-fold, or nine-fold, or at least 10-fold, or at least 20-fold longer time between at least two consecutive Cₘₐₓ peaks and/or Cₘᵢₙ troughs for blood levels of the fusion protein compared to the corresponding CF of the fusion protein not linked to the fusion protein and administered at a comparable dose regimen to a subject. In another embodiment of the foregoing, the administration of the fusion protein results in improvement in at least one measured parameter of a factor VIII-related disease using less frequent dosing or a lower total dosage in IUs of the fusion protein of the pharmaceutical composition compared to the corresponding biologically active protein component(s) not linked to the XTEN and administered to a subject using a therapeutically effective regimen to a subject.

The invention provides an isolated fusion protein comprising factor VIII and one or more XTEN, as described herein, used in the treatment of a coagulopathy. In one embodiment, the coagulopathy is hemophilia A, In another embodiment, the coagulopathy is a bleeding disorder. In another embodiment, the coagulopathy is caused by surgical intervention.

In another embodiment, the present invention provides kits, comprising packaging material and at least a first container comprising the pharmaceutical composition of the foregoing embodiment and a sheet of instructions for the reconstitution and/or administration of the pharmaceutical compositions to a subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features and advantages of the invention may be further explained by reference to the following detailed description and accompanying drawings that sets forth illustrative embodiments.

FIG. 1 shows a schematic representation of the FVIII architecture and spatial arrangement of the domains during processing and clotting, and is intended to represent both native FVIII and B domain deleted variants. The A1 domain ranges from residue 1 to 372 (numbering relative to the mature form of FVIII sequence NCBI Protein RefSeq NP_{—}000123), A2 domain ranges from residue 373 to 740, B domain ranges from residue 741 to 1648, A3 domain ranges from residue 1649 to 2019 (encompassing a3 acidic region), C1 2020 to 2172, C2 domain ranges from residue 2173 to 2332. BDD variants include deletions between the range 741 to 1648, leaving some or no remnant residues, with a non-limiting BDD remnant sequence being SFSQNPPVLKRHQR. FIG. 1A shows the domain architecture of a single chain FVIII prior to processing. Arrows indicate the sites at residues R372, R740, R1648, and R1689 that are cleaved in the processing and conversion of FVIII to FVIIIa. FIG. 1B shows the FVIII molecule that has been processed into the heterodimer by the cleavage at the R1648 residue, with the a3 acidic region of the A3 domain indicated on the N-terminus of the A3. FIG. 1C shows the FVIII molecule processed into the FVIIIa heterotrimer by the cleavage at the R372, R740, and R1689 residues.

FIG. 2 is a schematic of the coagulation cascade, showing the intrinsic and extrinsic arms leading to the common pathway.

FIG. 3 is a schematic of the logic flow chart of the algorithm SegScore. In the figure the following legend applies: i, j—counters used in the control loops that run through the entire sequence; HitCount—this variable is a counter that keeps track of how many times a subsequence encounters an identical subsequence in a block; SubSeqX—this variable holds the subsequence that is being checked for redundancy; SubSeqY—this variable holds the subsequence that the SubSeqX is checked against; BlockLen—this variable holds the user determined length of the block; SegLen—this variable holds the length of a segment. The program is hardcoded to generate scores for subsequences of lengths 3, 4, 5, 6, 7, 8, 9, and 10; Block—this variable holds a string of length BlockLen. The string is composed of letters from an input XTEN sequence and is determined by the position of the i counter; SubSeqList—this is a list that holds all of the generated subsequence scores.

FIG. 4 depicts the application of the algorithm SegScore to a hypothetical XTEN of 11 amino acids in order to determine the repetitiveness. An XTEN sequence consisting of N amino acids is divided into N−S+1 subsequences of length S (S=3 in this case). A pair-wise comparison of all subsequences is performed and the average number of identical subsequences is calculated to result in the subsequence score of 1.89.

FIG. 5 illustrates several examples of CFXTEN configurations of FVIII linked to XTEN (the latter shown as thick, wavy lines). In all cases, the FVIII can be either native or a BDD form of FVIII, or a single chain form in which the entire B domain, including the native cleavage sites are removed. FIG. 5A shows, left to right, three variations of single chain factor VIII with XTEN linked to the N-terminus, the C-terminus, and two XTEN linked to the N- and C-terminus. FIG. 5B shows six variations of mature heterodimer FVIII with, left to right, an XTEN linked to the N-terminus of the A1 domain; an XTEN linked to the C-terminus of the C2 domain; an XTEN linked to the N-terminus of the A1 domain and the C-terminus of the C2 domain; an XTEN linked to the N-terminus of the A1 domain and to the N-terminus of the A3 domain; an XTEN linked to the C-terminus of the C2 domain and to the N-terminus of the A3 domain via residual B domain amino acids; and an XTEN linked to the N-terminus of the A1 domain, the C-terminus of the A2 domain via residual B domain amino acids, and to the C-terminus of the C2 domain. FIG. 5C shows, left to right, three variations of single chain factor VIII: an XTEN linked to the N-terminus of the A1 domain, an XTEN linked within a surface loop of the A1 domain and an XTEN linked within a surface loop of the A3 domain; an XTEN linked within a surface loop of the A2 domain, an XTEN linked within a surface loop of the C2 domain and an XTEN linked to the C terminus of the C2 domain; an XTEN linked to the N-terminus of the A1 domain and within a surface loop of the C1 domain and to the C-terminus of the C domain. FIG. 5D shows six variations of mature heterodimer FVIII with, left to right, an XTEN linked to the N-terminus of the A1 domain, an XTEN linked within a surface loop of the A1 domain, and an XTEN linked within a surface loop of the A3 domain; an XTEN linked within a surface loop of the A2 domain, and an XTEN linked within a surface loop of the C1 domain, and an XTEN linked to the C-terminus of the C2 domain; an XTEN linked to the N-terminus of the A1 domain, an XTEN linked within a surface loop of the A1 domain, an XTEN linked within a surface loop of the A3 domain, and an XTEN linked to the C-terminus of the C2 domain; an XTEN linked to the N-terminus of the A1 domain, an XTEN linked to the N-terminus of the A3 domain via residual amino acids of the B domain, and an XTEN linked within a surface loop of the C2 domain; an XTEN linked within a surface loop of the A2 domain, an XTEN linked to the N-terminus of the A3 domain via residual amino acids of the B domain, an XTEN linked within a surface loop of the C1 domain, and an XTEN linked to the C-terminus of the C2 domain; and an XTEN linked within the B domain or between the residual B domain residues of the BDD variant (and the invention also contemplates a variation in which the XTEN replaces the entirety of the B domain, including all native cleavage sites, linking the A2 and A3 domains, resulting in a single chain form of factor VIII). This figure also embodies all variations in which one or more XTEN sequences are inserted within the 13 domain and the resulting fusions are cleaved at one or more sites (e.g., at R1648 site) during intracellular processing.

FIG. 6 is a graphic portrayal of the various analyses performed on a FVIII B-domain deleted sequence to identify insertion sites for XTEN within the FVIII sequence. Each of lines A-H are on an arbitrary scale of Y axis values across the FVIII BDD sequence such that low values represent areas with a high predicted tolerance for XTEN insertion, with the residue numbers on the X axis. Line A shows the domain boundaries; all discontinuities in this line represent boundaries that are likely to accept XTEN. Line B shows exon boundaries; i.e., each step in the line represents a new exon. Line C shown regions that were not visible in the X-ray structure due to a lack of order in the crystal. Lines labeled D represents multiple predictions of order that were calculated using the respective programs FoldIndex found on the World-Wide web site bip.weizmann.ac.il/fldbin/findex (last accessed Feb. 23, 2011) (see Jaime Prilusky, Clifford E. Felder, Tzviya Zeev-Ben-Mordehai, Edwin Rydberg, Orna Man, Jacques S. Beckmann, Israel Silman, and Joel L. Sussman, 2005, Bioinformatics based on the Kyte & Doolitlle algorithm, as well as RONN found on the World-Wide web site strubi.ox.ac.uk/RONN (last accessed Feb. 23, 2011) (see Yang, Z. R., Thomson, R., McMeil, P. and Esnouf, R. M. (2005) RONN: the bio-basis function neural network technique applied to the detection of natively disordered regions in proteins Bioinformatics 21: 3369-3376. Lines E and F were calculated based on multiple sequence alignments of FVIII genes from 11 mammals available in GenBank. Line E represents the conservation of individual residues. Line F represent the conservation of 3 amino acid segments of FVIII. Lines G and H represent gaps and insertions observed in the multiple sequence alignment of 11 mammalian FVIII genes. Line J lists the XTEN insertion points by amino acid number that were obtained based by combining the multiple measurements above.

FIG. 7 depicts the sites in a FVIII B-domain deleted sequence identified for insertion of XTEN using the information depicted in FIG. 6 and or Example 34. The amino acids with a double underline correspond to the specific insertion points of Table 5 or Table 25, while the amino acids with a single underline correspond to the span of amino acids around each insertion point that is considered suitable for insertion of XTEN between any two adjoining amino acids within the depicted span.

FIG. 8 is a schematic of the assembly of a CFXTEN library created by identifying insertion points as described for FIG. 6 followed by insertion of single XTEN (black bars) at the various insertion points using molecular biology techniques. The constructs are expressed and recovered, then evaluated for FVIII activity and pharmacokinetic properties to identify those CFXTEN configurations that result in enhanced properties.

FIG. 9 is a schematic of the assembly of a CFXTEN component library in which segments of FVIII BDD domains, either singly or linked to various lengths of XTEN (black bars) are assembled in a combinatorial fashion into libraries of genes encoding the CFXTEN, which can then be evaluated for FVIII activity and pharmacokinetic properties to identify those CFXTEN configurations that result in enhanced properties.

FIG. 10 illustrates several examples of CFXTEN configurations with XTEN (shown as thick, wavy lines), with certain XTEN releasable by inserting cleavage sequences (indicated by black triangles) that are cleavable by procoagulant proteases. FIG. 10A illustrates a scFVIII with two terminal releasable XTENS. FIG. 10B illustrates the same configuration as FIG. 10A but with an additional non-releasable XTEN linking the A3 and C1 domains. FIG. 10C illustrates a mature heterodimer FVIII with two terminal releasable XTEN. FIG. 10D illustrates the same configuration as 10C but with an additional non-releasable XTEN linking the A3 and C1 domains.

FIG. 11 is a schematic flowchart of representative steps in the assembly, production and the evaluation of an XTEN.

FIG. 12 is a schematic flowchart of representative steps in the assembly of a CFXTEN polynucleotide construct encoding a fusion protein. Individual oligonucleotides 501 are annealed into sequence motifs 502 such as a 12 amino acid motif (“12-mer”), which is ligated to additional sequence motifs from a library to create a pool that encompasses the desired length of the XTEN 504, as well as ligated to a smaller concentration of an oligo containing BbsI, and KpnI restriction sites 503. The resulting pool of ligation products is gel-purified and the band with the desired length of XTEN is cut, resulting in an isolated XTEN gene with a stopper sequence 505. The XTEN gene is cloned into a stuffer vector. In this case, the vector encodes an optional CBD sequence 506 and a GFP gene 508. Digestion is then performed with BbsI/HindIII to remove 507 and 508 and place the stop codon. The resulting product is then cloned into a BsaI/HindIII digested vector containing a gene encoding the FVIII, resulting in the gene 500 encoding an FVIII-XTEN fusion protein.

FIG. 13 is a schematic flowchart of representative steps in the assembly of a gene encoding fusion protein comprising a CF and XTEN, its expression and recovery as a fusion protein, and its evaluation as a candidate CFXTEN product.

FIG. 14 illustrates the use of donor XTEN sequences to produce truncated XTENs. FIG. 14A provides the sequence of AG864, with the underlined sequence used to generate AG576. FIG. 14B provides the sequence of AG864, with the underlined sequence used to generate AG288. FIG. 14C provides the sequence of AG864, with the underlined sequence used to generate AG144. FIG. 14D provides the sequence of AE864, with the underlined sequence used to generate AE576. FIG. 14E provides the sequence of AE864, with the underlined sequence used to generate AE288.

FIG. 15 is a schematic representation of the design of Factor VIII-XTEN expression vectors with different strategies introducing XTEN elements into the FVIII coding sequence. FIG. 15A shows an expression vector encoding XTEN fused to the 3′ end of the sequence encoding FVIII. FIG. 15B depicts an expression vector encoding an XTEN element inserted into the middle of the coding sequence of FVIII. FIG. 15C depicts an expression vector encoding two XTEN elements: one inserted into the FVIII coding sequence, and the other fused to the 3′ end of the FVIII coding sequence.

FIG. 16 illustrates the process of combinatorial gene assembly of genes encoding XTEN. In this case, the genes are assembled from 6 base fragments and each fragment is available in 4 different codon versions (A, B, C and D). This allows for a theoretical diversity of 4096 in the assembly of a 12 amino acid motif.

FIG. 17 shows the pharmacokinetic profile (plasma concentrations) in cynomolgus monkeys after single doses of different compositions of GFP linked to unstructured polypeptides of varying length, administered either subcutaneously or intravenously, as described in Example 28. The compositions were GFP-L288, GFP-L576, GFP-XTEN_AF576, GFP-Y576 and XTEN_AD836-GFP. Blood samples were analyzed at various times after injection and the concentration of GFP in plasma was measured by ELISA using a polyclonal antibody against GFP for capture and a biotinylated preparation of the same polyclonal antibody for detection. Results are presented as the plasma concentration versus time (h) after dosing and show, in particular, a considerable increase in half-life for the XTEN_AD836-GFP, the composition with the longest sequence length of XTEN. The construct with the shortest sequence length, the GFP-L288 had the shortest half-life.

FIG. 18 shows an SDS-PAGE gel of samples from a stability study of the fusion protein of XTEN_AE864 fused to the N-terminus of GFP (see Example 29). The GFP-XTEN was incubated in cynomolgus plasma and rat kidney lysate for up to 7 days at 37° C. In addition, GFP-XTEN administered to cynomolgus monkeys was also assessed. Samples were withdrawn at 0, 1 and 7 days and analyzed by SDS PAGE followed by detection using Western analysis with antibodies against GFP.

FIG. 19 shows results of a size exclusion chromatography analysis of glucagon-XTEN construct samples measured against protein standards of known molecular weight, with the graph output as absorbance versus retention volume, as described in Example 27. The glucagon-XTEN constructs are 1) glucagon-Y288; 2) glucagonY-144; 3) glucagon-Y72; and 4) glucagon-Y36. The results indicate an increase in apparent molecular weight with increasing length of XTEN moiety (see Example 27 for data). FIG. 20 shows results of a Western blot of proteins expressed by cell culture of cells transformed with constructs as designated. The samples in lanes 1-12 were: MW Standards, FVIII (42.5 ng), pBC0100B, pBC0114A, pBC0100, pBC0114, pBC0135, pBC0136, pBC0137, pBC0145, pBC0149, and pBC0146, respectively. Lanes 8, 9 and 12 show bands consistent with a FVIII with a C-terminal XTEN288, with an estimated MW of 95 kDa. Lanes 7 and 11 show bands consistent with a FVIII with a C-terminal XTEN42, with an estimated MW of 175 kDa. Lanes 2-6 show bands consistent with FVIII and heavy chain. Lanes 10 and 23 show bands consistent with heavy chain. Lane 7 shows a band consistent with heavy chain and an attached XTEN42.

FIG. 21 shows the results of FVIII assay on samples obtained from FVIII and von Willebrand factor double knock-out mice with hydrodynamic plasmid DNA injection, as detailed in Example 35,

### DETAILED DESCRIPTION OF THE INVENTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### DEFINITIONS

In the context of the present application, the following terms have the meanings ascribed to them unless specified otherwise:

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

The term “amino acid” refers to either natural and/or unnatural or synthetic amino acids, including but not limited to both the D or L optical isomers, and amino acid analogs and peptidomimetics. Standard single or three letter codes are used to designate amino acids.

The term “domain,” when used in reference to a factor VIII polypeptide refers to either a full length domain or a functional fragment thereof, for example, full length or functional fragments of the A1 domain, A2 domain, A3 domain, a3 domain, B domain, C1 domain, and/or C2 domain of factor VIII.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “non-naturally occurring,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. A number of scales have been developed. An example is a scale developed by Levitt, M, et al., J Mol Biol (1976) 104:59, which is listed in Hopp, T P, et al., Proc Natl Acad Sci USA (1981) 78:3824. Examples of “hydrophilic amino acids” are arginine, lysine, threonine, alanine, asparagine, and glutamine. Of particular interest are the hydrophilic amino acids aspartate, glutamate, and serine, and glycine. Examples of “hydrophobic amino acids” are tryptophan, tyrosine, phenylalanine, methionine, leucine, isoleucine, and valine.

A “fragment” when applied to a protein, is a truncated form of a native biologically active protein that retains at least a portion of the therapeutic and/or biological activity. A “variant”. when applied to a protein is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity compared with the reference biologically active protein. As used herein, the term “biologically active protein moiety” includes proteins modified deliberately, as for example, by site directed mutagenesis, synthesis of the encoding gene, insertions, or accidentally through mutations.

The term “sequence variant” means polypeptides that have been modified compared to their native or original sequence by one or more amino acid insertions, deletions, or substitutions. Insertions may be located at either or both termini of the protein, and/or may be positioned within internal regions of the amino acid sequence. A non-limiting example is insertion of an XTEN sequence within the sequence of the biologically-active payload protein. In deletion variants, one or more amino acid residues in a polypeptide as described herein are removed. Deletion variants, therefore, include all fragments of a payload polypeptide sequence. In substitution variants, one or more amino acid residues of a polypeptide are removed and replaced with alternative residues. In one aspect, the substitutions are conservative in nature and conservative substitutions of this type are well known in the art.

As used herein, “internal XTEN” refers to XTEN sequences that have been inserted into the sequence of the coagulation factor. Internal XTENs can be constructed by insertion of an XTEN sequence into the sequence of a coagulation factor such as FVIII, either by insertion between two adjacent amino acids or between two domains of the coagulation factor or wherein XTEN replaces a partial, internal sequence of the coagulation factor.

As used herein, “terminal XTEN” refers to XTEN sequences that have been fused to or in the N- or C-terminus of the coagulation factor or to a proteolytic cleavage sequence or linker at the N- or C-terminus of the coagulation factor. Terminal XTENs can be fused to the native termini of the coagulation factor. Alternatively, terminal XTENs can replace a terminal sequence of the coagulation factor.

The term “XTEN release site” refers to a cleavage sequence in CFXTEN fusion proteins that can be recognized and cleaved by a mammalian protease, effecting release of an XTEN or a portion of an XTEN from the CFXTEN fusion protein. As used herein, “mammalian protease” means a protease that normally exists in the body fluids, cells or tissues of a mammal. XTEN release sites can be engineered to be cleaved by various mammalian proteases (a.k.a. “XTEN release proteases”) such as FXIa, FXIIa, kallikrein, FVIIIa, FVIIIa, FXa, FIIa (thrombin), Elastase-2, MMP-12, MMP13, MMP-17, MMP-20, or any protease that is present during a clotting event. Other equivalent proteases (endogenous or exogenous) that are capable of recognizing a defined cleavage site can be utilized. The cleavage sites can be adjusted and tailored to the protease utilized.

“Activity” as applied to form(s) of a CFXTEN polypeptide provided herein, refers to retention of a procoagulant activity with reference to a native FVIII coagulation factor derived from human plasma, whereas “biological activity” refers to an in vitro or in vivo biological function or effect, including but not limited to either receptor or ligand binding; or an effect on coagulation generally known in the art for the FVIII coagulation factor, or a cellular, physiologic, or clinical response, including arrest of a bleeding episode.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein contains at least one fusion polypeptide comprising at least one region in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”, “linked,” “fused,” and “fusion” are used interchangeably herein. These terms refer to the joining together of two or more chemical elements, sequences or components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. An “in-frame fusion” refers to the joining of two or more open reading frames (ORFS) to form a continuous longer ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature).

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” or “sequence identity” refers to sequence similarity or interchangeability between two or more polynucleotide sequences or between two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity compared to those sequences. Polypeptides that are homologous preferably have sequence identities of at least 80%, or at least 90%, or at least 95%, or at least 97%, or at least 98%, or have at least 99% sequence identity when sequences of comparable length are optimally aligned.

“Ligation” refers to the process of forming phosphodiester bonds between two nucleic acid fragments or genes, linking them together. To ligate the DNA fragments or genes together, the ends of the DNA must be compatible with each other. In some cases, the ends will be directly compatible after endonuclease digestion. However, it may be necessary to first convert the staggered ends commonly produced after endonuclease digestion to blunt ends to make them compatible for ligation.

The terms “stringent conditions” or “stringent hybridization conditions” includes reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60° C. to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al., “Molecular Cloning: A Laboratory Manual,” 3^{rd} edition, Cold Spring Harbor Laboratory Press, 2001. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “non-repetitiveness” as used herein in the context of a polypeptide refers to a lack or limited degree of internal homology in a peptide or polypeptide sequence. The term “substantially non-repetitive” can mean, for example, that there are few or no instances of four contiguous amino acids in the sequence that are identical amino acid types or that the polypeptide has a average subsequence score (defined infra) of 3 or less or that there isn't a pattern in the order, from N- to C-terminus, of the sequence motifs that constitute the polypeptide sequence. The term “repetitiveness” as used herein in the context of a polypeptide refers to the degree of internal homology in a peptide or polypeptide sequence. In contrast, a “repetitive” sequence may contain multiple identical copies of short amino acid sequences. For instance, a polypeptide sequence of interest may be divided into n-mer sequences and the number of identical sequences can be counted. Highly repetitive sequences contain a large fraction of identical sequences while non-repetitive sequences contain few identical sequences. In the context of a polypeptide, a sequence can contain multiple copies of shorter sequences of defined or variable length, or motifs, in which the motifs themselves have non-repetitive sequences, rendering the full-length polypeptide substantially non-repetitive. The length of polypeptide within which the non-repetitiveness is measured can vary from 3 amino acids to about 200 amino acids, about from 6 to about 50 amino acids, or from about 9 to about 14 amino acids. “Repetitiveness” used in the context of polynucleotide sequences refers to the degree of internal homology in the sequence such as, for example, the frequency of identical nucleotide sequences of a given length. Repetitiveness can, for example, be measured by analyzing the frequency of identical sequences.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

“Serum degradation resistance,” as applied to a polypeptide, refers to the ability of the polypeptides to withstand degradation in blood or components thereof, which typically involves proteases in the serum or plasma. The serum degradation resistance can be measured by combining the protein with human (or mouse, rat, monkey, as appropriate) serum or plasma, typically for a range of days (e.g. 0.25, 0.5, 1, 2, 4, 8, 16 days), typically at about 37° C. The samples for these time points can be run on a Western blot assay and the protein is detected with an antibody. The antibody can be to a tag in the protein. If the protein shows a single band on the western, where the protein's size is identical to that of the injected protein, then no degradation has occurred. In this exemplary method, the time point where 50% of the protein is degraded, as judged by Western blots or equivalent techniques, is the serum degradation half-life or “serum half-life” of the protein.

The term “t_{1/2}” as used herein means the terminal half-life calculated as ln(2)/Kₑₗ. Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. The terms “t_{1/2}”, “terminal half-life”, “elimination half-life” and “circulating half-life” are used interchangeably herein.

“Active clearance” means the mechanisms by which CF is removed from the circulation other than by filtration or coagulation, and which includes removal from the circulation mediated by cells, receptors, metabolism, or degradation of the FVIII.

“Apparent molecular weight factor” and “apparent molecular weight” are related terms referring to a measure of the relative increase or decrease in apparent molecular weight exhibited by a particular amino acid sequence. The apparent molecular weight is determined using size exclusion chromatography (SEC) or similar methods by comparing to globular protein standards, and is measured in “apparent kD” units. The apparent molecular weight factor is the ratio between the apparent molecular weight and the actual molecular weight; the latter predicted by adding, based on amino acid composition, the calculated molecular weight of each type of amino acid in the composition or by estimation from comparison to molecular weight standards in an SDS electrophoresis gel.

The terms “hydrodynamic radius” or “Stokes radius” is the effective radius (Rₕ in nm) of a molecule in a solution measured by assuming that it is a body moving through the solution and resisted by the solution's viscosity. In the embodiments of the invention, the hydrodynamic radius measurements of the XTEN fusion proteins correlate with the ‘apparent molecular weight factor’, which is a more intuitive measure. The “hydrodynamic radius” of a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

“Physiological conditions” refers to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers are listed in Sambrook et al. (2001). Physiologically relevant temperature ranges from about 25° C. to about 38° C., and preferably from about 35° C. to about 37° C.

A “reactive group” is a chemical structure that can be coupled to a second reactive group. Examples for reactive groups are amino groups, carboxyl groups, sulfhydryl groups, hydroxyl groups, aldehyde groups, azide groups. Some reactive groups can be activated to facilitate coupling with a second reactive group. Non-limiting examples for activation are the reaction of a carboxyl group with carbodiimide, the conversion of a carboxyl group into an activated ester, or the conversion of a carboxyl group into an azide function.

“Controlled release agent”, “slow release agent”, “depot formulation” and “sustained release agent” are used interchangeably to refer to an agent capable of extending the duration of release of a polypeptide of the invention relative to the duration of release when the polypeptide is administered in the absence of agent. Different embodiments of the present invention may have different release rates, resulting in different therapeutic amounts.

The terms “antigen”, “target antigen” and “immunogen” are used interchangeably herein to refer to the structure or binding determinant that an antibody fragment or an antibody fragment-based therapeutic binds to or has specificity against.

The term “payload” as used herein refers to a protein or peptide sequence that has biological or therapeutic activity; the counterpart to the pharmacophore of small molecules. Examples of payloads include, but are not limited to, coagulation factors, cytokines, enzymes, hormones, and blood and growth factors. Payloads can further comprise genetically fused or chemically conjugated moieties such as chemotherapeutic agents, antiviral compounds, toxins, or contrast agents. These conjugated moieties can be joined to the rest of the polypeptide via a linker that may be cleavable or non-cleavable.

The term “antagonist”, as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present invention, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

As used herein, “treat” or “treating,” or “palliating” or “ameliorating” are used interchangeably and mean administering a drug or a biologic to achieve a therapeutic benefit, to cure or reduce the severity of an existing disease, disorder or condition, or to achieve a prophylactic benefit, prevent or reduce the likelihood of onset or severity the occurrence of a disease, disorder or condition. By therapeutic benefit is meant eradication or amelioration of the underlying disorder being treated or one or more of the physiological symptoms associated with the underlying disorder such that an improvement is observed in the subject, notwithstanding that the subject may still be afflicted with the underlying disorder.

A “therapeutic effect” or “therapeutic benefit,” as used herein, refers to a physiologic effect, including but not limited to the cure, mitigation, amelioration, or prevention of disease in humans or other animals, or to otherwise enhance physical or mental wellbeing of humans or animals, caused by a fusion polypeptide of the invention other than the ability to induce the production of an antibody against an antigenic epitope possessed by the biologically active protein. For prophylactic benefit, the compositions may be administered to a subject at risk of developing a particular disease or condition, or to a subject reporting one or more of the physiological symptoms of a disease, even though a diagnosis of this disease may not have been made.

The terms “therapeutically effective amount” and “therapeutically effective dose”, as used herein, refer to an amount of a drug or a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “therapeutically effective dose regimen”, as used herein, refers to a schedule for consecutively administered multiple doses (i.e., at least two or more) of a biologically active protein, either alone or as a part of a fusion protein composition, wherein the doses are given in therapeutically effective amounts to result in sustained beneficial effect on any symptom, aspect, measured parameter or characteristics of a disease state or condition.

### I). General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al. eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11^{th} Edition, McGraw-Hill, 2005; and Freshney, R. I., “Culture of Animal Cells: A Manual of Basic Technique,” 4^{th} edition, John Wiley & Sons, Somerset, N.J., 2000, the contents of which are incorporated in their entirety herein by reference.

### II). Coagulation Factor VIII

The present invention relates, in part, to compositions comprising factor VIII coagulation factor (CF) linked to one or more extended recombinant proteins (XTEN), resulting in a CFXTEN fusion protein composition. As used herein, “CF” refers to factor VIII (FVIII) or mimetics, sequence variants and truncated versions of FVIII, as described below.

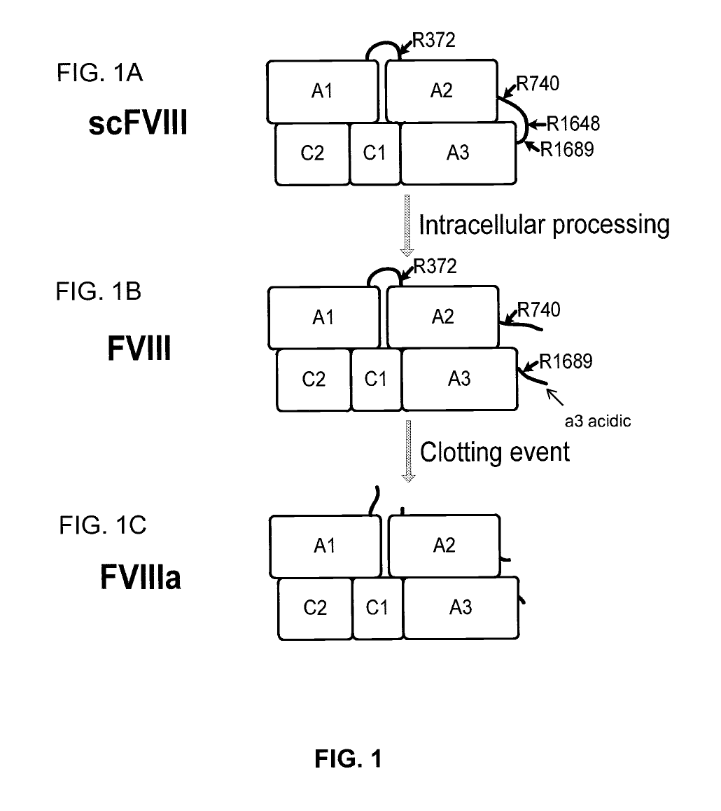

“Factor VIII” or “FVIII” or “FVIII polypeptide” means a blood coagulation factor protein and species and sequence variants thereof that includes, but is not limited to, the 2351 amino acid single-chain precursor protein (with a 19-amino acid hydrophobic signal peptide), the mature 2332 amino acid factor VIII cofactor protein of approximately 270-330 kDa with the domain structure A1-A2-B-A3-C1-C2, as well as the nonenzymatic “active” or cofactor form of FVIII (FVIIIa) that is a circulating heterodimer of two chains that form as a result of proteolytic cleavage after R1648 of a heavy chain form composed of A1-A2-B (in the range of 90-220 kD) of amino acids 1-1648 (numbered relative to the mature FVIII form) and a light chain A3-C1-C2 of 80 kDa of amino acids 1649-2232, each of which is depicted schematically in FIG. 1. Further, the A3 domain encompasses, at its N-terminus, an a3 acidic region. As used herein, “Factor VIII” or “FVIII” or “FVIII polypeptide” also includes variant forms, including proteins with substitutions, additions and/or deletions so long as the variant retains a desired biological activity such as procoagulant activity. In one embodiment, the human Factor VIII domains are defined by the following amino acid residues: A1, residues Ala1-Arg372; A2, residues Ser373-Arg740; B, residues Ser741-Arg1648; A3, residues Ser1649-Asn2019; C1, residues Lys2020-Asn2172; C2, residues Ser2173-Tyr2332. The A3-C1-C2 sequence includes residues Ser1649-Tyr2332. In another embodiment, residues Glu1649-Arg1689, is usually referred to as the a3 acidic region. In certain embodiments, the a3 acidic region is a part of the A3 domain. Such Factor VIII include truncated sequences such as B-domain deleted “BDD” sequences in which a portion or the majority of the B domain sequence is deleted (such as BDD sequences disclosed or referenced in U.S. Pat. Nos. 6,818,439 and 7,632,921), sequences that include heterologous amino acid insertions or substitutions (such as aspartic acid substituted for valine at position 75), or single chain FVIII (scFVIII) in which the heavy and light chains are covalently connected by a linker. As used herein, “FVIII” shall be any functional form of factor VIII molecule with the typical characteristics of blood coagulation factor VIII capable of e.g., correcting human factor VIII deficiencies when administered to such a subject, e.g., a subject with hemophilia A. FVIII or sequence variants have been isolated, characterized, and cloned, as described in U.S. Pat. Nos. 4,757,006; 4,965,199; 5,004,804; 5,198,349, 5,250,421; 5,919,766; 6,228,620; 6,818,439; 7,138,505; 7,632,921; and 20100081615.

Human factor VIII is encoded by a single-copy gene residing at the tip of the long arm of the X chromosome (q28). It comprises nearly 186,000 base pairs (bp) and constitutes approximately 0.1% of the X-chromosome (White, G. C. and Shoemaker, C. B., Blood (1989) 73:1-12). The DNA encoding the mature factor VIII mRNA is found in 26 separate exons ranging in size from 69 to 3,106 bp. The 25 intervening intron regions that separate the exons range in size from 207 to 32,400 bp. The complete gene consists of approximately 9 kb of exon and 177 kb of intron. The three repeat A domains have approximately 30% sequence homology. The B domain contains 19 of the approximately 25 predicted glycosylation sites, and the following A3 domain is believed to contain the binding site for the von Willebrand factor. The tandem C domains follow the A3 domain, and have approximately 37% homology to each other (White, G. C. and Shoemaker, C. B., Blood (1989) 73:1-12).

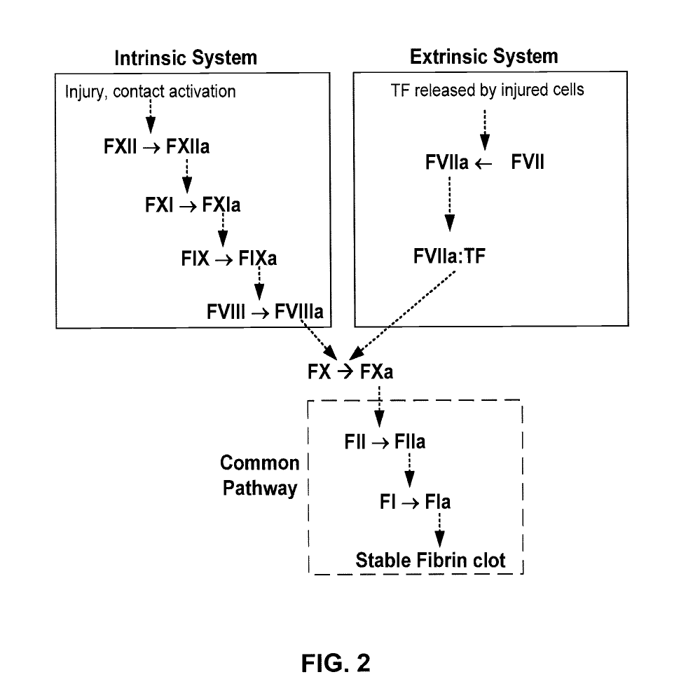

The B domain separates the A2 and A3 domains of native factor FVIII in the newly synthesized precursor single-chain molecule. The precise boundaries of the B domain have been variously reported as extending from amino acids 712 to 1648 of the precursor sequence (Wood et al., Nature (1984) 312:330-337) or amino acids 741-1648 (Pipe, S W, Haemophilia (2009) 15:1187-1196 and U.S. Pat. No. 7,560,107) or amino acids 740-1689 (Toole, J J, Proc. Natl. Acad. Sci. USA (1986) 83:5939-5942). As used herein, “B domain” used herein means amino acids 741-1648 of mature Factor VIII. As used herein, “FVIII B domain deletion” or “FVIII BDD” means a FVIII sequence with any, a fragment of, or all of amino acids 741 to 1648 deleted. In one embodiment, FVIII BDD variants retain remnant amino acids of the B domain from the N-terminal end (“B1” as used herein) and C-terminal end (“B2” as used herein). In one FVIII BDD variant, the B domain remnant amino acids are SFSQNPPVLKRHQR. In one FVIII BDD variant, the B1 remant is SFS and the B2 remant is QNPPVLKRHQR. In another FVIII BDD variant, the B1 remant is SFSQN and the B2 remant is PPVLKRHQR. A “B-domain-deleted Factor VIII,” “FVIII BDD,” or “BDD FVIII” may have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563, each of which is incorporated herein by reference in its entirety. In some embodiments, a B-domain-deleted Factor VIII sequence of the present invention comprises any one of the deletions disclosed at col. 4, line 4 to col. 5, line 28 and examples 1-5 of U.S. Pat. No. 6,316,226 (also in U.S. Pat. No. 6,346,513). In another embodiment, a B-domain deleted Factor VIII is the S743/Q1638 B-domain deleted Factor VIII (SQ version Factor VIII) (e.g., Factor VIII having a deletion from amino acid 744 to amino acid 1637, e.g., Factor VIII having amino acids 1-743 and amino acids 1638-2332 of full-length Factor VIII). In some embodiments, a B-domain-deleted Factor VIII of the present invention has a deletion disclosed at col. 2, lines 26-51 and examples 5-8 of U.S. Pat. No. 5,789,203 (also U.S. Pat. No. 6,060,447, U.S. Pat. No. 5,595,886, and U.S. Pat. No. 6,228,620). In some embodiments, a B-domain-deleted Factor VIII has a deletion described in col. 1, lines 25 to col. 2, line 40 of U.S. Pat. No. 5,972,885; col. 6, lines 1-22 and example 1 of U.S. Pat. No. 6,048,720; col. 2, lines 17-46 of U.S. Pat. No. 5,543,502; col. 4, line 22 to col. 5, line 36 of U.S. Pat. No. 5,171,844; col. 2, lines 55-68, FIG. 2, and example 1 of U.S. Pat. No. 5,112,950; col. 2, line 2 to col. 19, line 21 and table 2 of U.S. Pat. No. 4,868,112; col. 2, line 1 to col. 3, line 19, col. 3, line 40 to col. 4, line 67, col. 7, line 43 to col. 8, line 26, and col. 11, line 5 to col. 13, line 39 of U.S. Pat. No. 7,041,635; or col. 4, lines 25-53, of U.S. Pat. No. 6,458,563. In some embodiments, a B-domain-deleted Factor VIII has a deletion of most of the B domain, but still contains amino-terminal sequences of the B domain that are essential for in vivo proteolytic processing of the primary translation product into two polypeptide chain, as disclosed in WO 91/09122, which is incorporated herein by reference in its entirety. In some embodiments, a B-domain-deleted Factor VIII is constructed with a deletion of amino acids 747-1638, i.e., virtually a complete deletion of the B domain. Hoeben R. C., et al. J. Biol. Chem. 265 (13): 7318-7323 (1990), incorporated herein by reference in its entirety. A B-domain-deleted Factor VIII may also contain a deletion of amino acids 771-1666 or amino acids 868-1562 of Factor VIII. Meulien P., et al. Protein Eng. 2(4): 301-6 (1988), incorporated herein by reference in its entirety. Additional B domain deletions that are part of the invention include: deletion of amino acids 982 through 1562 or 760 through 1639 (Toole et al., Proc. Natl. Acad. Sci. U.S.A. (1986) 83, 5939-5942)), 797 through 1562 (Eaton, et al. Biochemistry (1986) 25:8343-8347)), 741 through 1646 (Kaufman (PCT published application No. WO 87/04187)), 747-1560 (Sarver, et al., DNA (1987) 6:553-564)), 741 though 1648 (Pasek (PCT application No. 88/00831)), or 816 through 1598 or 741 through 1648 (Lagner (Behring Inst. Mitt. (1988) No 82:16-25, EP 295597)), each of which is incorporated herein by reference in its entirety. Each of the foregoing deletions may be made in any Factor VIII sequence.

Proteins involved in clotting include factor I, factor II, factor III, factor IV, factor V, factor VI, factor VII, factor VIII, factor IX, factor X, factor XI, factor XII, factor XIII, Protein C, and tissue factor (collectively or individually “clotting protein(s)”). The interaction of the major clotting proteins in the intrinsic and extrinsic clotting pathways is showed in FIG. 2. The majority of the clotting proteins are present in zymogen form, but when activated, exhibit a procoagulant protease activity in which they activate another of the clotting proteins, contributing to the intrinsic or extrinsic coagulation pathway and clot formation. In the intrinsic pathway of the coagulation cascade, FVIII associates with a complex of activated factor IX, factor X, calcium, and phospholipid. The factor VIII heterodimer has no enzymatic activity, but the heterodimer becomes active as a cofactor of the enzyme factor IXa after proteolytic activation by thrombin or factor Xa, with the activity of factor VIIIa characterized by its ability to form a membrane binding site for factors IXa and X in a conformation suitable for activation of the factor X by factor IXa.

The activated cofactor, factor VIIIa, is a heterotrimer comprised of the A1 domain and the A2 domain and the light chain including domains A3-C1-C2. The activation of factor IX is achieved by a two-step removal of the activation peptide (Ala 146-Arg 180) from the molecule (Bajaj et al., Human factor IX and factor IXa, in METHODS IN ENZYMOLOGY. 1993). The first cleavage is made at the Arg 145-Ala 146 site by either factor XIa or factor VIIa/tissue factor. The second, and rate limiting cleavage is made at Arg 180-Val 181. The activation removes 35 residues. Activated human factor IX exists as a heterodimer of the C-terminal heavy chain (28 kDa) and an N-terminal light chain (18 kDa), which are held together by one disulfide bridge attaching the enzyme to the Gla domain. Factor IXa in turn activates factor X in concert with activated factor VIII. Alternatively, factors IX and X can both be activated by factor Vila complexed with lipidated tissue factor, generated via the extrinsic pathway. Factor Xa then participates in the final common pathway whereby prothrombin is converted to thrombin, and thrombin, in turn converts fibrinogen to fibrin to form the clot.

Defects in the coagulation process can lead to bleeding disorders in which the time taken for clot formation is prolonged. Such defects can be congenital or acquired. For example, hemophilia A and B are inherited diseases characterized by deficiencies in FVIII and FIX, respectively. Stated differently, biologically active factor VIII corrects the coagulation defect in plasma derived from individuals afflicted with hemophilia A. Recombinant FVIII has been shown to be effective and has been approved for the treatment of hemophilia A in adult and pediatric patients, and also is used to stop bleeding episodes or prevent bleeding associated with trauma and/or surgery. Current therapeutic uses of factor VIII can be problematic in the treatment of individuals exhibiting a deficiency in factor VIII, as well as those individuals with Von Willebrand's disease. In addition, individuals receiving factor VIII in replacement therapy frequently develop antibodies to these proteins. Continuing treatment is exceedingly difficult because of the presence of these antibodies that reduce or negate the efficacy of the treatment.

In one aspect, the invention contemplates inclusion of FVIII sequences in the CFXTEN fusion protein compositions that are identical to human FVIII, sequences that have homology to FVIII sequences, sequences that are natural, such as from humans, non-human primates, mammals (including domestic animals); all of which retain at least a portion of the procoagulant activity of native FVIII and that are useful for preventing, treating, mediating, or ameliorating hemophilia A or bleeding episodes related to trauma, surgery, or deficiency of coagulation factor VIII. “Procoagulant activity” as used herein refers to an activity that promotes clot formation, whether in an in vitro assay or in vivo. Sequences with homology to FVIII may be found by standard homology searching techniques, such as NCBI BLAST, or in public databases such as Chemical Abstracts Services Databases (e.g., the CAS Registry), GenBank, The Universal Protein Resource (UniProt) and subscription provided databases such as GenSeq (e.g., Derwent)

In one embodiment, the FVIII incorporated into the subject CFXTEN compositions is a recombinant polypeptide with a sequence corresponding to a FVIII protein found in nature. In another embodiment, the FVIII is a non-natural FVIII sequence variant, fragment, homolog, or a mimetic of a natural sequence that retains at least a portion of the procoagulant activity of the corresponding native FVIII. In another embodiment, the FVIII is a truncated variant with all or a portion of the B domain deleted (“FVIII BDD”), which can be in either heterodimeric form or can remain as a single chain (“scFVIII”), the latter described in Meulien et al., Protein Eng. (1988) 2(4):301-306. In another embodiment, heterologous sequences are incorporated into the FVIII, which may include XTEN, as described more fully below. Table 1 and Table 31 provide a non-limiting list of amino acid sequences of FVIII that are encompassed by the CFXTEN fusion proteins of the invention. In some embodiments, FVIII incorporated into CFXTEN fusion proteins include proteins that have at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity compared to an amino acid sequence of comparable length selected from Table 1.

TABLE 1
FVIII amino acid sequences

| Name | |
|---|---|
| (source) | Amino Acid Sequence |
| FVIII | MQIELSTCFFLCLLRFCFSATRRYYLGAVELSWDYMQSDLGELPVDARFPPRVPKSFPFN |
| precursor | TSVVYKKTLFVEFTDHLFNIAKPRPPWMGLLGPTIQAEVYDTVVITLKNMASHPVSLHA |
| polypeptide | VGVSYWKASEGAEYDDQTSQREKEDDKVFPGGSHTYVWQVLKENGPMASDPLCLTYS |
| (human) | YLSHVDLVKDLNSGLIGALLVCREGSLAKEKTQTLHKFILLFAVFDEGKSWHSETKNSL |
| | MQDRDAASARAWPKMHTVNGYVNRSLPGLIGCHRKSVYWHVIGMGTTPEVHSIFLEG |
| | HTFLVRNHRQASLEISPITFLTAQTLLMDLGQFLLFCHISSHQHDGMEAYVKVDSCPEEP |
| | QLRMKNNEEAEDYDDDLTDSEMDVVRFDDDNSPSFIQIRSVAKKHPKTWVHYIAAEEE |
| | DWDYAPLVLAPDDRSYKSQYLNNGPQRIGRKYKKVRFMAYTDETFKTREAIQHESGIL |
| | GPLLYGEVGDTLLIIFKNQASRPYNIYPHGITDVRPLYSRRLPKGVKHLKDFPILPGEIFKY |
| | KWTVTVEDGPTKSDPRCLTRYYSSFVNMERDLASGLIGPLLICYKESVDQRGNQIMSDK |
| | RNVILFSVFDENRSWYLTENIQRFLPNPAGVQLEDPEFQASNIMHSINGYVFDSLQLSVC |
| | LHEVAYWYILSIGAQTDFLSVFFSGYTFKHKMVYEDTLTLFPFSGETVFMSMENPGLWI |
| | LGCHNSDFRNRGMTALLKVSSCDKNTGDYYEDSYEDISAYLLSKNNAIEPRSFSQNSRH |
| | PSTRQKQFNATTIPENDIEKTDPWFAHRTPMPKIQNVSSSDLLMLLRQSPTPHGLSLSDLQ |
| | EAKYETFSDDPSPGAIDSNNSLSEMTHFRPQLHHSGDMVFTPESGLQLRLNEKLGTTAAT |
| | ELKKLDFKVSSTSNNLISTIPSDNLAAGTDNTSSLGPPSMPVHYDSQLDTTLFGKKSSPLT |
| | ESGGPLSLSEENNDSKLLESGLMNSQESSWGKNVSSTESGRLFKGKRAHGPALLTKDNA |
| | LFKVSISLLKTNKTSNNSATNRKTHIDGPSLLIENSPSVWQNILESDTEFKKVTPLIHDRM |
| | LMDKNATALRLNHMSNKTTSSKNMEMVQQKKEGPIPPDAQNPDMSFFKMLFLPESAR |
| | WIQRTHGKNSLNSGQGPSPKQLVSLGPEKSVEGQNFLSEKNKVVVGKGEFTKDVGLKE |
| | MVFPSSRNLFLTNLDNLHENNTHNQEKKIQEEIEKKETLIQENVVLPQIHTVTGTKNFMK |
| | NLFLLSTRQNVEGSYDGAYAPVLQDFRSLNDSTNRTKKHTAHFSKKGEEENLEGLGNQ |
| | TKQIVEKYACTTRISPNTSQQNFVTQRSKRALKQFRLPLEETELEKRIIVDDTSTQWSKN |
| | MKHLTPSTLTQIDYNEKEKGAITQSPLSDCLTRSHSIPQANRSPLPIAKVSSFPSIRPIYLTR |
| | VLFQDNSSHLPAASYRKKDSGVQESSHFLQGAKKNNLSLAILTLEMTGDQREVGSLGTS |
| | ATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQ |
| | EKSPEKTAFKKKDTILSLNACESNHAIAAINEGQNKPEIEVTWAKQGRTERLCSQNPPVL |
| | KRHQREITRTTLQSDQEEIDYDDTISVEMKKEDFDIYDEDENQSPRSFQKKTRHYFIAAV |
| | ERLWDYGMSSSPHVLRNRAQSGSVPQFKKVVFQEFTDGSFTQPLYRGELNEHLGLLGP |
| | YIRAEVEDNIMVTFRNQASRPYSFYSSLISYEEDQRQGAEPRKNFVKPNETKTYFWKVQ |
| | HHMAPTKDEFDCKAWAYFSDVDLEKDVHSGLIGPLLVCHTNTLNPAHGRQVTVQEFAL |
| | FFTIFDETKSWYFTENMERNCRAPCNIQMEDPTFKENYRFHAINGYIMDTLPGLVMAQD |
| | QRIRWYLLSMGSNENIHSIHFSGHVFTVRKKEEYKMALYNLYPGVFETVEMLPSKAGIW |
| | RVECLIGEHLHAGMSTLFLVYSNKCQTPLGMASGHIRDFQITASGQYGQWAPKLARLH |
| | YSGSINAWSTKEPFSWIKVDLLAPMIIHGIKTQGARQKFSSLYISQFIIMYSLDGKKWQTY |
| | RGNSTGTLMVFFGNVDSSGIKHNIFNPPIIARYIRLHPTHYSIRSTLRMELMGCDLNSCSM |
| | PLGMESKAISDAQITASSYFTNMFATWSPSKARLHLQGRSNAWRPQVNNPKEWLQVDF |
| | QKTMKVTGVTTQGVKSLLTSMYVKEFLISSSQDGHQWTLFFQNGKVKVFQGNQDSFTP |
| | VVNSLDPPLLTRYLRIHPQSWVHQIALRMEVLGCEAQDLY |
| FVIII mature | ATRRYYLGAVELSWDYMQSDLGELPVDARFPPRVPKSFPFNTSVVYKKTLFVEFTDHLF |
| (human) | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQNSRHPSTRQKQFNATTIPENDIEKT |
| | DPWFAHRTPMPKIQNVSSSDLLMLLRQSPTPHGLSLSDLQEAKYETFSDDPSPGAIDSNN |
| | SLSEMTHFRPQLHHSGDMVFTPESGLQLRLNEKLGTTAATELKKLDFKVSSTSNNLISTIP |
| | SDNLAAGTDNTSSLGPPSMPVHYDSQLDTTLFGKKSSPLTESGGPLSLSEENNDSKLLES |
| | GLMNSQESSWGKNVSSTESGRLFKGKRAHGPALLTKDNALFKVSISLLKTNKTSNNSAT |
| | NRKTHIDGPSLLIENSPSVWQNILESDTEFKKVTPLIHDRMLMDKNATALRLNHMSNKT |
| | TSSKNMEMVQQKKEGPIPPDAQNPDMSFFKMLFLPESARWIQRTHGKNSLNSGQGPSPK |
| | QLVSLGPEKSVEGQNFLSEKNKVVVGKGEFTKDVGLKEMVFPSSRNLFLTNLDNLHEN |
| | NTHNQEKKIQEEIEKKETLIQENVVLPQIHTVTGTKNFMKNLFLLSTRQNVEGSYDGAY |
| | APVLQDFRSLNDSTNRTKKHTAHFSKKGEEENLEGLGNQTKQIVEKYACTTRISPNTSQ |
| | QNFVTQRSKRALKQFRLPLEETELEKRIIVDDTSTQWSKNMKHLTPSTLTQIDYNEKEKG |
| | AITQSPLSDCLTRSHSIPQANRSPLPIAKVSSFPSIRPIYLTRVLFQDNSSHLPAASYRKKDS |
| | GVQESSHFLQGAKKNNLSLAILTLEMTGDQREVGSLGTSATNSVTYKKVENTVLPKPDL |
| | PKTSGKVELLPKVHIYQKDLFPTETSNGSPGHLDLVEGSLLQGTEGAIKWNEANRPGKV |
| | PFLRVATESSAKTPSKLLDPLAWDNHYGTQIPKEEWKSQEKSPEKTAFKKKDTILSLNAC |
| | ESNHAIAAINEGQNKPEIEVTWAKQGRTERLCSQNPPVLKRHQREITRTTLQSDQEEIDY |
| | DDTISVEMKKEDFDIYDEDENQSPRSFQKKTRHYFIAAVERLWDYGMSSSPHVLRNRAQ |
| | SGSVPQFKKVVFQEFTDGSFTQPLYRGELNEHLGLLGPYIRAEVEDNIMVTFRNQASRPY |
| | SFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCR |
| | APCNIQMEDPTFKENYRFHAINGYIMDTLPGLVMAQDQRIRWYLLSMGSNENIHSIHFS |
| | GHVFTVRKKEEYKMALYNLYPGVFETVEMLPSKAGIWRVECLIGEHLHAGMSTLFLVY |
| | SNKCQTPLGMASGHIRDFQITASGQYGQWAPKLARLHYSGSINAWSTKEPFSWIKVDLL |
| | APMIIHGIKTQGARQKFSSLYISQFIIMYSLDGKKWQTYRGNSTGTLMVFFGNVDSSGIK |
| | HNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTS |
| | MYVKEFLISSSQDGHQWTLFFQNGKVKVFQGNQDSFTPVVNSLDPPLLTRYLRIHPQSW |
| | VHQIALRMEVLGCEAQDLY |
| FVIII (Canine) | MQVELYTCCFLCLLPFSLSATRKYYLGAVELSWDYMQSDLLSALHADTSFSSRVPGSLP |
| | LTTSVTYRKTVFVEFTDDLFNIAKPRPPWMGLLGPTIQAEVYDTVVIVLKNMASHPVSL |
| | HAVGVSYWKASEGAEYEDQTSQKEKEDDNVIPGESHTYVWQVLKENGPMASDPPCLT |
| | YSYFSHVDLVKDLNSGLIGALLVCKEGSLAKERTQTLQEFVLLFAVFDEGKSWHSETNA |
| | SLTQAEAQHELHTINGYVNRSLPGLTVCHKRSVYWHVIGMGTTPEVHSIFLEGHTFLVR |
| | NHRQASLEISPITFLTAQTFLMDLGQFLLFCHIPSHQHDGMEAYVKVDSCPEEPQLRMKN |
| | NEDKDYDDGLYDSDMDVVSFDDDSSSPFIQIRSVAKKHPKTWVHYIAAEEEDWDYAPS |
| | GPTPNDRSHKNLYLNNGPQRIGKKYKKVRFVAYTDETFKTREAIQYESGILGPLLYGEV |
| | GDTLLIIFKNQASRPYNIYPHGINYVTPLHTGRLPKGVKHLKDMPILPGEIFKYKWTVTV |
| | EDGPTKSDPRCLTRYYSSFINLERDLASGLIGPLLICYKESVDQRGNQMMSDKRNVILFS |
| | VFDENRSWYLTENMQRFLPNADVVQPHDPEFQLSNIMHSINGYVFDNLQLSVCLHEVA |
| | YWYILSVGAQTDFLSVFFSGYTFKHKMVYEDTLTLFPFSGETVFMSMENPGLWVLGCH |
| | NSDFRNRGMTALLKVSSCNRNIDDYYEDTYEDIPTPLLNENNVIKPRSFSQNSRHPSTKE |
| | KQLKATTTPENDIEKIDLQSGERTQLIKAQSVSSSDLLMLLGQNPTPRGLFLSDLREATDR |
| | ADDHSRGAIERNKGPPEVASLRPELRHSEDREFTPEPELQLRLNENLGTNTTVELKKLDL |
| | KISSSSDSLMTSPTIPSDKLAAATEKTGSLGPPNMSVHFNSHLGTIVFGNNSSHLIQSGVPL |
| | ELSEEDNDSKLLEAPLMNIQESSLRENVLSMESNRLFKEERIRGPASLIKDNALFKVNISS |
| | VKTNRAPVNLTTNRKTRVAIPTLLIENSTSVWQDIMLERNTEFKEVTSLIHNETFMDRNT |
| | TALGLNHVSNKTTLSKNVEMAHQKKEDPVPLRAENPDLSSSKIPFLPDWIKTHGKNSLS |
| | SEQRPSPKQLTSLGSEKSVKDQNFLSEEKVVVGEDEFTKDTELQEIFPNNKSIFFANLANV |
| | QENDTYNQEKKSPEEIERKEKLTQENVALPQAHTMIGTKNFLKNLFLLSTKQNVAGLEE |
| | QPYTPILQDTRSLNDSPHSEGIHMANFSKIREEANLEGLGNQTNQMVERFPSTTRMSSNA |
| | SQHVITQRGKRSLKQPRLSQGEIKFERKVIANDTSTQWSKNMNYLAQGTLTQIEYNEKE |
| | KRAITQSPLSDCSMRNHVTIQMNDSALPVAKESASPSVRHTDLTKIPSQHNSSHLPASAC |
| | NYTFRERTSGVQEGSHFLQEAKRNNLSLAFVTLGITEGQGKFSSLGKSATNQPMYKKLE |
| | NTVLLQPGLSETSDKVELLSQVHVDQEDSFPTKTSNDSPGHLDLMGKIFLQKTQGPVKM |
| | NKTNSPGKVPFLKWATESSEKIPSKLLGVLAWDNHYDTQIPSEEWKSQKKSQTNTAFKR |
| | KDTILPLGPCENNDSTAAINEGQDKPQREAMWAKQGEPGRLCSQNPPVSKHHQREITVT |
| | TLQPEEDKFEYDDTFSIEMKREDFDIYGDYENQGLRSFQKKTRHYFIAAVERLWDYGMS |
| | RSPHILRNRAQSGDVQQFKKVVFQEFTDGSFTQPLYRGELNEHLGLLGPYIRAEVEDNIV |
| | VTFKNQASRPYSFYSSLISYDEDEGQGAEPRRKFVNPNETKIYFWKVQHHMAPTKDEFD |
| | CKAWAYFSDVDLEKDVHSGLIGPLLICRSNTLNPAHGRQVTVQEFALVFTIFDETKSWY |
| | FTENLERNCRAPCNVQKEDPTLKENFRFHAINGYVKDTLPGLVMAQDQKVRWYLLSM |
| | GSNENIHSIHFSGHVFTVRKKEEYKMAVYNLYPGVFETVEMLPSQVGIWRIECLIGEHLQ |
| | AGMSTLFLVYSKKCQTPLGMASGHIRDFQITASGQYGQWAPKLARLHYSGSINAWSTK |
| | DPFSWIKVDLLAPMIIHGIMTQGARQKFSSLYVSQFIIMYSLDGNKWHSYRGNSTGTLM |
| | VFFGNVDSSGIKHNIFNPPIIAQYIRLHPTHYSIRSTLRMELLGCDFNSCSMPLGMESKAIS |
| | DAQITASSYLSSMLATWSPSQARLHLQGRTNAWRPQANNPKEWLQVDFRKTMKVTGIT |
| | TQGVKSLLISMYVKEFLISSSQDGHNWTLFLQNGKVKVFQGNRDSSTPVRNRLEPPLVA |
| | RYVRLHPQSWAHHIALRLEVLGCDTQQPA |
| FVIII (Pig) | ATRRYYLGAVELSWDYMQSDLGELPVDARFPPRVPKSFPFNTSVVYKKTLFVEFTDHLF |
| | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQNSRHPSTRQKQFNATTIPENDIEKT |
| | DPWFAHRTPMPKIQNVSSSDLLMLLRQSPTPHGLSLSDLQEAKYETFSDDPSPGAIDSNN |
| | SLSEMTHFRPQLHHSGDMVFTPESGLQLRLNEKLGTTAATELKKLDFKVSSTSNNLISTIP |
| | SDNLAAGTDNTSSLGPPSMPVHYDSQLDTTLFGKKSSPLTESGGPLSLSEENNDSKLLES |
| | GLMNSQESSWGKNVSSTESGRLFKGKRAHGPALLTKDNALFKVSISLLKTNKTSNNSAT |
| | NRKTHIDGPSLLIENSPSVWQNILESDTEFKKVTPLIHDRMLMDKNATALRLNHMSNKT |
| | TSSKNMEMVQQKKEGPIPPDAQNPDMSFFKMLFLPESARWIQRTHGKNSLNSGQGPSPK |
| | QLVSLGPEKSVEGQNFLSEKNKVVVGKGEFTKDVGLKEMVFPSSRNLFLTNLDNLHEN |
| | NTHNQEKKIQEEIEKKETLIQENVVLPQIHTVTGTKNFMKNLFLLSTRQNVEGSYDGAY |
| | APVLQDFRSLNDSTNRTKKHTAHFSKKGEEENLEGLGNQTKQIVEKYACTTRISPNTSQ |
| | QNFVTQRSKRALKQFRLPLEETELEKRIIVDDTSTQWSKNMKHLTPSTLTQIDYNEKEKG |
| | AITQSPLSDCLTRSHSIPQANRSPLPIAKVSSFPSIRPIYLTRVLFQDNSSHLPAASYRKKDS |
| | GVQESSHFLQGAKKNNLSLAILTLEMTGDQREVGSLGTSATNSVTYKKVENTVLPKPDL |
| | PKTSGKVELLPKVHIYQKDLFPTETSNGSPGHLDLVEGSLLQGTEGAIKWNEANRPGKV |
| | PFLRVATESSAKTPSKLLDPLAWDNHYGTQIPKEEWKSQEKSPEKTAFKKKDTILSLNAC |
| | ESNHAIAAINEGQNKPEIEVTWAKQGRTERLCSQNPPVLKRHQREITRTTLQSDQEEIDY |
| | DDTISVEMKKEDFDIYDEDENQSPRSFQKKTRHYFIAAVERLWDYGMSSSPHVLRNRAQ |
| | SGSVPQFKKVVFQEFTDGSFTQPLYRGELNEHLGLLGPYIRAEVEDNIMVTFRNQASRPY |
| | SFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCR |
| | APCNIQMEDPTFKENYRFHAINGYIMDTLPGLVMAQDQRIRWYLLSMGSNENIHSIHFS |
| | GHVFTVRKKEEYKMALYNLYPGVFETVEMLPSKAGIWRVECLIGEHLHAGMSTLFLVY |
| | SNKCQTPLGMASGHIRDFQITASGQYGQWAPKLARLHYSGSINAWSTKEPFSWIKVDLL |
| | APMIIHGIKTQGARQKFSSLYISQFIIMYSLDGKKWQTYRGNSTGTLMVFFGNVDSSGIK |
| | HNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTS |
| | MYVKEFLISSSQDGHQWTLFFQNGKVKVFQGNQDSFTPVVNSLDPPLLTRYLRIHPQSW |
| | VHQIALRMEVLGCEAQDLY |
| FVIII (Mouse) | AIRRYYLGAVELSWNYIQSDLLSVLHTDSRFLPRMSTSFPFNTSIMYKKTVFVEYKDQLF |
| | NIAKPRPPWMGLLGPTIWTEVHDTVVITLKNMASHPVSLHAVGVSYWKASEGDEYEDQ |
| | TSQMEKEDDKVFPGESHTYVWQVLKENGPMASDPPCLTYSYMSHVDLVKDLNSGLIG |
| | ALLVCKEGSLSKERTQMLYQFVLLFAVFDEGKSWHSETNDSYTQSMDSASARDWPKM |
| | HTVNGYVNRSLPGLIGCHRKSVYWHVIGMGTTPEIHSIFLEGHTFFVRNHRQASLEISPIT |
| | FLTAQTLLIDLGQFLLFCHISSHKHDGMEAYVKVDSCPEESQWQKKNNNEEMEDYDDD |
| | LYSEMDMFTLDYDSSPFIQIRSVAKKYPKTWIHYISAEEEDWDYAPSVPTSDNGSYKSQ |
| | YLSNGPHRIGRKYKKVRFIAYTDETFKTRETIQHESGLLGPLLYGEVGDTLLIIFKNQASR |
| | PYNIYPHGITDVSPLHARRLPRGIKHVKDLPIHPGEIFKYKWTVTVEDGPTKSDPRCLTRY |
| | YSSFINPERDLASGLIGPLLICYKESVDQRGNQMMSDKRNVILFSIFDENQSWYITENMQ |
| | RFLPNAAKTQPQDPGFQASNIMHSINGYVFDSLELTVCLHEVAYWHILSVGAQTDFLSIF |
| | FSGYTFKHKMVYEDTLTLFPFSGETVFMSMENPGLWVLGCHNSDFRKRGMTALLKVSS |
| | CDKSTSDYYEEIYEDIPTQLVNENNVIDPRSFFQNTNHPNTRKKKFKDSTIPKNDMEKIEP |
| | QFEEIAEMLKVQSVSVSDMLMLLGQSHPTPHGLFLSDGQEAIYEAIHDDHSPNAIDSNEG |
| | PSKVTQLRPESHHSEKIVFTPQPGLQLRSNKSLETTIEVKWKKLGLQVSSLPSNLMTTTIL |
| | SDNLKATFEKTDSSGFPDMPVHSSSKLSTTAFGKKAYSLVGSHVPLNASEENSDSNILDS |
| | TLMYSQESLPRDNILSIENDRLLREKRFHGIALLTKDNTLFKDNVSLMKTNKTYNHSTTN |
| | EKLHTESPTSIENSTTDLQDAILKVNSEIQEVTALIHDGTLLGKNSTYLRLNHMLNRTTST |
| | KNKDIFHRKDEDPIPQDEENTIMPFSKMLFLSESSNWFKKTNGNNSLNSEQEHSPKQLVY |
| | LMFKKYVKNQSFLSEKNKVTVEQDGFTKNIGLKDMAFPHNMSIFLTTLSNVHENGRHN |
| | QEKNIQEEIEKEALIEEKVVLPQVHEATGSKNFLKDILILGTRQNISLYEVHVPVLQNITSI |
| | NNSTNTVQIHMEHFFKRRKDKETNSEGLVNKTREMVKNYPSQKNITTQRSKRALGQFR |
| | LSTQWLKTINCSTQCIIKQIDHSKEMKKFITKSSLSDSSVIKSTTQTNSSDSHIVKTSAFPPI |
| | DLKRSPFQNKFSHVQASSYIYDFKTKSSRIQESNNFLKETKINNPSLAILPWNMFIDQGKF |
| | TSPGKSNTNSVTYKKRENIIFLKPTLPEESGKIELLPQVSIQEEEILPTETSHGSPGHLNLM |
| | KEVFLQKIQGPTKWNKAKRHGESIKGKTESSKNTRSKLLNHHAWDYHYAAQIPKDMW |
| | KSKEKSPEIISIKQEDTILSLRPHGNSHSIGANEKQNWPQRETTWVKQGQTQRTCSQIPPV |
| | LKRHQRELSAFQSEQEATDYDDAITIETIEDFDIYSEDIKQGPRSFQQKTRHYFIAAVERL |
| | WDYGMSTSHVLRNRYQSDNVPQFKKVVFQEFTDGSFSQPLYRGELNEHLGLLGPYIRA |
| | EVEDNIMVTFKNQASRPYSFYSSLISYKEDQRGEEPRRNFVKPNETKIYFWKVQHHMAP |
| | TEDEFDCKAWAYFSDVDLERDMHSGLIGPLLICHANTLNPAHGRQVSVQEFALLFTIFD |
| | ETKSWYFTENVKRNCKTPCNFQMEDPTLKENYRFHAINGYVMDTLPGLVMAQDQRIR |
| | WYLLSMGNNENIQSIHFSGHVFTVRKKEEYKMAVYNLYPGVFETLEMIPSRAGIWRVEC |
| | LIGEHLQAGMSTLFLVYSKQCQIPLGMASGSIRDFQITASGHYGQWAPNLARLHYSGSIN |
| | AWSTKEPFSWIKVDLLAPMIVHGIKTQGARQKFSSLYISQFIIMYSLDGKKWLSYQGNST |
| | GTLMVFFGNVDSSGIKHNSFNPPIIARYIRLHPTHSSIRSTLRMELMGCDLNSCSIPLGMES |
| | KVISDTQITASSYFTNMFATWSPSQARLHLQGRTNAWRPQVNDPKQWLQVDLQKTMK |
| | VTGIITQGVKSLFTSMFVKEFLISSSQDGHHWTQILYNGKVKVFQGNQDSSTPMMNSLD |
| | PPLLTRYLRIHPQIWEHQIALRLEILGCEAQQQY |
| FVIII BDD | MQIELSTCFFLCLLRFCFSATRRYYLGAVELSWDYMQSDLGELPVDARFPPRVPKSFPFN |
| variant (U.S. | TSVVYKKTLFVEFTVHLFNIAKPRPPWMGLLGPTIQAEVYDTVVITLKNMASHPVSLHA |
| Pat. No. | VGVSYWKASEGAEYDDQTSQREKEDDKVFPGGSHTYVWQVLKENGPMASDPLCLTYS |
| 7,632,921, SEQ | YLSHVDLVKDLNSGLIGALLVCREGSLAKEKTQTLHKFILLFAVFDEGKSWHSETKNSL |
| ID NO: 3) | MQDRDAASARAWPKMHTVNGYVNRSLPGLIGCHRKSVYWHVIGMGTTPEVHSIFLEG |
| | HTFLVRNHRQASLEISPITFLTAQTLLMDLGQFLLFCHISSHQHDGMEAYVKVDSCPEEP |
| | QLRMKNNEEAEDYDDDLTDSEMDVVRFDDDNSPSFIQIRSVAKKHPKTWVHYIAAEEE |
| | DWDYAPLVLAPDDRSYKSQYLNNGPQRIGRKYKKVRFMAYTDETFKTREAIQHESGIL |
| | GPLLYGEVGDTLLIIFKNQASRPYNIYPHGITDVRPLYSRRLPKGVKHLKDFPILPGEIFKY |
| | KWTVTVEDGPTKSDPRCLTRYYSSFVNMERDLASGLIGPLLICYKESVDQRGNQIMSDK |
| | RNVILFSVFDENRSWYLTENIQRFLPNPAGVQLEDPEFQASNIMHSINGYVFDSLQLSVC |
| | LHEVAYWYILSIGAQTDFLSVFFSGYTFKHKMVYEDTLTLFPFSGETVFMSMENPGLWI |
| | LGCHNSDFRNRGMTALLKVSSCDKNTGDYYEDSYEDISAYLLSKNNAIEPRSFSQNPPV |
| | LKRHQREITRTTLQSDQEEIDYDDTISVEMKKEDFDIYDEDENQSPRSFQKKTRHYFIAA |
| | VERLWDYGMSSSPHVLRNRAQSGSVPQFKKVVFQEFTDGSFTQPLYRGELNEHLGLLG |
| | PYIRAEVEDNIMVTFRNQASRPYSFYSSLISYEEDQRQGAEPRKNFVKPNETKTYFWKV |
| | QHHMAPTKDEFDCKAWAYFSDVDLEKDVHSGLIGPLLVCHTNTLNPAHGRQVTVQEF |
| | ALFFTIFDETKSWYFTENMERNCRAPCNIQMEDPTFKENYRFHAINGYIMDTLPGLVMA |
| | QDQRIRWYLLSMGSNENIHSIHFSGHVFTVRKKEEYKMALYNLYPGVFETVEMLPSKA |
| | GIWRVECLIGEHLHAGMSTLFLVYSNKCQTPLGMASGHIRDFQITASGQYGQWAPKLA |
| | RLHYSGSINAWSTKEPFSWIKVDLLAPMIIHGIKTQGARQKFSSLYISQFIIMYSLDGKKW |
| | QTYRGNSTGTLMVFFGNVDSSGIKHNIFNPPIIARYIRLHPTHYSIRSTLRMELMGCDLNS |
| | CSMPLGMESKAISDAQITASSYFTNMFATWSPSKARLHLQGRSNAWRPQVNNPKEWLQ |
| | VDFQKTMKVTGVTTQGVKSLLTSMYVKEFLISSSQDGHQWTLFFQNGKVKVFQGNQD |
| | SFTPVVNSLDPPLLTRYLRIHPQSWVHQIALRMEVLGCEAQDLY |
| FVIII BDD-2 | ATRRYYLGAVELSWDYMQSDLGELPVDARFPPRVPKSFPFNTSVVYKKTLFVEFTVHLF |
| | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQNPPVLKRHQREITRTTLQSDQEEID |
| | YDDTISVEMKKEDFDIYDEDENQSPRSFQKKTRHYFIAAVERLWDYGMSSSPHVLRNRA |
| | QSGSVPQFKKVVFQEFTDGSFTQPLYRGELNEHLGLLGPYIRAEVEDNIMVTFRNQASRP |
| | YSFYSSLISYEEDQRQGAEPRKNFVKPNETKTYFWKVQHHMAPTKDEFDCKAWAYFSD |
| | VDLEKDVHSGLIGPLLVCHTNTLNPAHGRQVTVQEFALFFTIFDETKSWYFTENMERNC |
| | RAPCNIQMEDPTFKENYRFHAINGYIMDTLPGLVMAQDQRIRWYLLSMGSNENIHSIHFS |
| | GHVFTVRKKEEYKMALYNLYPGVFETVEMLPSKAGIWRVECLIGEHLHAGMSTLFLVY |
| | SNKCQTPLGMASGHIRDFQITASGQYGQWAPKLARLHYSGSINAWSTKEPFSWIKVDLL |
| | APMIIHGIKTQGARQKFSSLYISQFIIMYSLDGKKWQTYRGNSTGTLMVFFGNVDSSGIK |
| | HNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTS |
| | MYVKEFLISSSQDGHQWTLFFQNGKVKVFQGNQDSFTPVVNSLDPPLLTRYLRIHPQSW |
| | VHQIALRMEVLGCEAQDLY |
| FVIII BDD-3 | ATRRYYLGAVELSWDYMQSDLGELPVDARFPPRVPKSFPFNTSVVYKKTLFVEFTVHLF |
| (G1648) | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQNPPVLKRHQGEITRTTLQSDQEEID |
| | YDDTISVEMKKEDFDIYDEDENQSPRSFQKKTRHYFIAAVERLWDYGMSSSPHVLRNRA |
| | QSGSVPQFKKVVFQEFTDGSFTQPLYRGELNEHLGLLGPYIRAEVEDNIMVTFRNQASRP |
| | YSFYSSLISYEEDQRQGAEPRKNFVKPNETKTYFWKVQHHMAPTKDEFDCKAWAYFSD |
| | VDLEKDVHSGLIGPLLVCHTNTLNPAHGRQVTVQEFALFFTIFDETKSWYFTENMERNC |
| | RAPCNIQMEDPTFKENYRFHAINGYIMDTLPGLVMAQDQRIRWYLLSMGSNENIHSIHFS |
| | GHVFTVRKKEEYKMALYNLYPGVFETVEMLPSKAGIWRVECLIGEHLHAGMSTLFLVY |
| | SNKCQTPLGMASGHIRDFQITASGQYGQWAPKLARLHYSGSINAWSTKEPFSWIKVDLL |
| | APMIIHGIKTQGARQKFSSLYISQFIIMYSLDGKKWQTYRGNSTGTLMVFFGNVDSSGIK |
| | HNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTS |
| | MYVKEFLISSSQDGHQWTLFFQNGKVKVFQGNQDSFTPVVNSLDPPLLTRYLRIHPQSW |
| | VHQIALRMEVLGCEAQDLY |
| FVIII BDD-4 | ATRRYYLGAVELSWDYMQSDLGELPVDARFPPRVPKSFPFNTSVVYKKTLFVEFTVHLF |
| | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQQSPRSFQKKTRHYFIAAVERLWDY |
| | GMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPT |
| | KDEFDCKAWAYFSDVDLEKDVHSGLIGPLLVCHTNTLNPAHGRQVTVQEFALFFTIFDE |
| | TKSWYFTENMERNCRAPCNIQMEDPTFKENYRFHAINGYIMDTLPGLVMAQDQRIRWY |
| | LLSMGSNENIHSIHFSGHVFTVRKKEEYKMALYNLYPGVFETVEMLPSKAGIWRVECLI |
| | GEHLHAGMSTLFLVYSNKCQTPLGMASGHIRDFQITASGQYGQWAPKLARLHYSGSIN |
| | AWSTKEPFSWIKVDLLAPMIIHGIKTQGARQKFSSLYISQFIIMYSLDGKKWQTYRGNST |
| | GTLMVFFGNVDSSGIKHNIFNPPIIARYIRLHPTHYSIRSTLRMELMGCDLNSCSMPLGME |
| | SKAISDAQITASSYFTNMFATWSPSKARLHLQGRSNAWRPQVNNPKEWLQVDFQKTMK |
| | VTGVTTQGVKSLLTSMYVKEFLISSSQDGHQWTLFFQNGKVKVFQGNQDSFTPVVNSL |
| | DPPLLTRYLRIHPQSWVHQIALRMEVLGCEAQDLY |
| FVIII BDD-5 | ATRRYYLGAVELSWDYMQSDLGELPVDARFPPRVPKSFPFNTSVVYKKTLFVEFTVHLF |
| | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKQSPRSFQKKTRHYFIAAVERLWDYGMSSSPHVLRNRAQSGSVPQFKKVVFQEFT |
| | DGSFTQPLYRGELNEHLGLLGPYIRAEVEDNIMVTFRNQASRPYSFYSSLISYEEDQRQG |
| | AEPRKNFVKPNETKTYFWKVQHHMAPTKDEFDCKAWAYFSDVDLEKDVHSGLIGPLL |
| | VCHTNTLNPAHGRQVTVQEFALFFTIFDETKSWYFTENMERNCRAPCNIQMEDPTFKEN |
| | YRFHAINGYIMDTLPGLVMAQDQRIRWYLLSMGSNENIHSIHFSGHVFTVRKKEEYKM |
| | ALYNLYPGVFETVEMLPSKAGIWRVECLIGEHLHAGMSTLFLVYSNKCQTPLGMASGHI |
| | RDFQITASGQYGQWAPKLARLHYSGSINAWSTKEPFSWIKVDLLAPMIIHGIKTQGARQ |
| | KFSSLYISQFIIMYSLDGKKWQTYRGNSTGTLMVFFGNVDSSGIKHNIFNPPIIARYIRLHP |
| | THYSIRSTLRMELMGCDLNSCSMPLGMESKAISDAQITASSYFTNMFATWSPSKARLHL |
| | QGRSNAWRPQVNNPKEWLQVDFQKTMKVTGVTTQGVKSLLTSMYVKEFLISSSQDGH |
| | QWTLFFQNGKVKVFQGNQDSFTPVVNSLDPPLLTRYLRIHPQSWVHQIALRMEVLGCE |
| | AQDLY |
| FVIII BDD-6 | ATRRYYLGAVELSWDYMQSDLGELPVDARFPPRVPKSFPFNTSVVYKKTLFVEFTDHLF |
| | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQNSRHPSTRQKQFNATTIPENDIEKT |
| | DTISVEMKKEDFDIYDEDENQSPRSFQKKTRHYFIAAVERLWDYGMSSSPHVLRNRAQS |
| | GSVPQFKKVVFQEFTDGSFTQPLYRGELNEHLGLLGPYIRAEVEDNIMVTFRNQASRPYS |
| | FYSSLISYEEDQRQGAEPRKNFVKPNETKTYFWKVQHHMAPTKDEFDCKAWAYFSDVD |
| | LEKDVHSGLIGPLLVCHTNTLNPAHGRQVTVQEFALFFTIFDETKSWYFTENMERNCRA |
| | PCNIQMEDPTFKENYRFHAINGYIMDTLPGLVMAQDQRIRWYLLSMGSNENIHSIHFSG |
| | HVFTVRKKEEYKMALYNLYPGVFETVEMLPSKAGIWRVECLIGEHLHAGMSTLFLVYS |
| | NKCQTPLGMASGHIRDFQITASGQYGQWAPKLARLHYSGSINAWSTKEPFSWIKVDLLA |
| | PMIIHGIKTQGARQKFSSLYISQFIIMYSLDGKKWQTYRGNSTGTLMVFFGNVDSSGIKH |
| | NIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTS |
| | MYVKEFLISSSQDGHQWTLFFQNGKVKVFQGNQDSFTPVVNSLDPPLLTRYLRIHPQSW |
| | VHQIALRMEVLGCEAQDLY |
| FVIII BDD-7 | ATRRYYLGAVELSWDYMQSDLGELPVDARFPPRVPKSFPFNTSVVYKKTLFVEFTVHLF |
| | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQSPRSFQKKTRHYFIAAVERLWDYG |
| | MSSSPHVLRNRAQSGSVPQFKKVVFQEFTDGSFTQPLYRGELNEHLGLLGPYIRAEVED |
| | NIMVTFRNQASRPYSFYSSLISYEEDQRQGAEPRKNFVKPNETKTYFWKVQHHMAPTK |
| | DEFDCKAWAYFSDVDLEKDVHSGLIGPLLVCHTNTLNPAHGRQVTVQEFALFFTIFDET |
| | KSWYFTENMERNCRAPCNIQMEDPTFKENYRFHAINGYIMDTLPGLVMAQDQRIRWYL |
| | LSMGSNENIHSIHFSGHVFTVRKKEEYKMALYNLYPGVFETVEMLPSKAGIWRVECLIG |
| | EHLHAGMSTLFLVYSNKCQTPLGMASGHIRDFQITASGQYGQWAPKLARLHYSGSINA |
| | WSTKEPFSWIKVDLLAPMIIHGIKTQGARQKFSSLYISQFIIMYSLDGKKWQTYRGNSTG |
| | TLMVFFGNVDSSGIKHNIFNPPIIARYIRLHPTHYSIRSTLRMELMGCDLNSCSMPLGMES |
| | KAISDAQITASSYFTNMFATWSPSKARLHLQGRSNAWRPQVNNPKEWLQVDFQKTMK |
| | VTGVTTQGVKSLLTSMYVKEFLISSSQDGHQWTLFFQNGKVKVFQGNQDSFTPVVNSL |
| | DPPLLTRYLRIHPQSWVHQIALRMEVLGCEAQDLY |
| FVIII BDD-8 | MQIELSTCFFLCLLRFCFSATRRYYLGAVELSWDYMQSDLGELPVDARFPPRVPKSFPFN |
| precursor | TSVVYKKTLFVEFTDHLFNIAKPRPPWMGLLGPTIQAEVYDTVVITLKNMASHPVSLHA |
| (U.S. Pat. No. | VGVSYWKASEGAEYDDQTSQREKEDDKVFPGGSHTYVWQVLKENGPMASDPLCLTYS |
| 6,818,439 SEQ | YLSHVDLVKDLNSGLIGALLVCREGSLAKEKTQTLHKFILLFAVFDEGKSWHSETKNSL |
| ID NO: 47) | MQDRDAASARAWPKMHTVNGYVNRSLPGLIGCHRKSVYWHVIGMGTTPEVHSIFLEG |
| | HTFLVRNHRQASLEISPITFLTAQTLLMDLGQFLLFCHISSHQHDGMEAYVKVDSCPEEP |
| | QLRMKNNEEAEDYDDDLTDSEMDVVRFDDDNSPSFIQIRSVAKKHPKTWVHYIAAEEE |
| | DWDYAPLVLAPDDRSYKSQYLNNGPQRIGRKYKKVRFMAYTDETFKTREAIQHESGIL |
| | GPLLYGEVGDTLLIIFKNQASRPYNIYPHGITDVRPLYSRRLPKGVKHLKDFPILPGEIFKY |
| | KWTVTVEDGPTKSDPRCLTRYYSSFVNMERDLASGLIGPLLICYKESVDQRGNQIMSDK |
| | RNVILFSVFDENRSWYLTENIQRFLPNPAGVQLEDPEFQASNIMHSINGYVFDSLQLSVC |
| | LHEVAYWYILSIGAQTDFLSVFFSGYTFKHKMVYEDTLTLFPFSGETVFMSMENPGLWI |
| | LGCHNSDFRNRGMTALLKVSSCDKNTGDYYEDSYEDISAYLLSKNNAIEPRSFSQNPPV |
| | LKRHQREITRTTLQSDQEEIDYDDTISVEMKKEDFDIYDEDENQSPRSFQKKTRHYFIAA |
| | VERLWDYGMSSSPHVLRNRAQSGSVPQFKKVVFQEFTDGSFTQPLYRGELNEHLGLLG |
| | PYIRAEVEDNIMVTFRNQASRPYSFYSSLISYEEDQRQGAEPRKNFVKPNETKTYFWKV |
| | QHHMAPTKDEFDCKAWAYFSDVDLEKDVHSGLIGPLLVCHTNTLNPAHGRQVTVQEF |
| | ALFFTIFDETKSWYFTENMERNCRAPCNIQMEDPTFKENYRFHAINGYIMDTLPGLVMA |
| | QDQRIRWYLLSMGSNENIHSIHFSGHVFTVRKKEEYKMALYNLYPGVFETVEMLPSKA |
| | GIWRVECLIGEHLHAGMSTLFLVYSNKCQTPLGMASGHIRDFQITASGQYGQWAPKLA |
| | RLHYSGSINAWSTKEPFSWIKVDLLAPMIIHGIKTQGARQKFSSLYISQFIIMYSLDGKKW |
| | QTYRGNSTGTLMVFFGNVDSSGIKHNIFNPPIIARYIRLHPTHYSIRSTLRMELMGCDLNS |
| | CSMPLGMESKAISDAQITASSYFTNMFATWSPSKARLHLQGRSNAWRPQVNNPKEWLQ |
| | VDFQKTMKVTGVTTQGVKSLLTSMYVKEFLISSSQDGHQWTLFFQNGKVKVFQGNQD |
| | SFTPVVNSLDPPLLTRYLRIHPQSWVHQIALRMEVLGCEAQDLY |
| FVIII BDD-9 | ATRRYYLGAVELSWDYMQSDLGELPVDARFPPRVPKSFPFNTSVVYKKTLFVEFTDHLF |
| mature | NIAKPRPPWMGLLGPTIQAEVYDTVVITLKNMASHPVSLHAVGVSYWKASEGAEYDDQ |
| (U.S. Pat. No. | TSQREKEDDKVFPGGSHTYVWQVLKENGPMASDPLCLTYSYLSHVDLVKDLNSGLIGA |
| 6,818,439) | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQNPPVLKRHQREITRTTLQSDQEEID |
| | YDDTISVEMKKEDFDIYDEDENQSPRSFQKKTRHYFIAAVERLWDYGMSSSPHVLRNRA |
| | QSGSVPQFKKVVFQEFTDGSFTQPLYRGELNEHLGLLGPYIRAEVEDNIMVTFRNQASRP |
| | YSFYSSLISYEEDQRQGAEPRKNFVKPNETKTYFWKVQHHMAPTKDEFDCKAWAYFSD |
| | VDLEKDVHSGLIGPLLVCHTNTLNPAHGRQVTVQEFALFFTIFDETKSWYFTENMERNC |
| | RAPCNIQMEDPTFKENYRFHAINGYIMDTLPGLVMAQDQRIRWYLLSMGSNENIHSIHFS |
| | GHVFTVRKKEEYKMALYNLYPGVFETVEMLPSKAGIWRVECLIGEHLHAGMSTLFLVY |
| | SNKCQTPLGMASGHIRDFQITASGQYGQWAPKLARLHYSGSINAWSTKEPFSWIKVDLL |
| | APMIIHGIKTQGARQKFSSLYISQFIIMYSLDGKKWQTYRGNSTGTLMVFFGNVDSSGIK |
| | HNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTS |
| | MYVKEFLISSSQDGHQWTLFFQNGKVKVFQGNQDSFTPVVNSLDPPLLTRYLRIHPQSW |
| | VHQIALRMEVLGCEAQDLY |

The present invention also contemplates CFXTEN comprising FVIII with various amino acid deletions, insertions and substitutions made in the FVIII sequences of Table 1 and Table 31 that retain procoagulant activity. Examples of conservative substitutions for amino acids in polypeptide sequences are shown in Table 2. In embodiments of the CFXTEN in which the sequence identity of the FVIII is less than 100% compared to a specific sequence disclosed herein, the invention contemplates substitution of any of the other 19 natural L-amino acids for a given amino acid residue of the given FVIII, which may be at any position within the sequence of the FVIII, including adjacent amino acid residues. If any one substitution results in an undesirable change in procoagulant activity, then one of the alternative amino acids can be employed and the construct protein evaluated by the methods described herein (e.g., the assays of Table 27), or using any of the techniques and guidelines for conservative and non-conservative mutations set forth, for instance, in U.S. Pat. No. 5,364,934, the content of which is incorporated by reference in its entirety, or using methods generally known in the art. In addition, variants can include, for instance, polypeptides wherein one or more amino acid residues are added or deleted at the N- or C-terminus of the full-length native amino acid sequence or of a domain of a FVIII that retains some if not all of the procoagulant activity of the native peptide, e.g., the ability to associate with another coagulation factor and/or participate in the coagulation cascade, leading to fibrin formation and hemostasis. The resulting FVIII sequences that retain at least a portion (e.g., at least 10%, 20%, 30%, 40%, 50%, 60%, 70%, 80%, 90%, or at least 95% or more) of the procoagulant activity in comparison to native circulating FVIII are considered useful for the fusion protein compositions of this invention. Such FVIII variants are known in the art, including those described in U.S. Pat. Nos. 6,316,226; 6,818,439; 7,632,921; 20080227691, which are incorporated herein by reference. In one embodiment, a FVIII sequence variant has an aspartic acid substituted for valine at amino acid position 75 (numbered relative to the native mature form of FVIII).

TABLE 2
Exemplary conservative amino acid substitutions

| Original Residue | Exemplary Substitutions |
|---|---|
| Ala (A) | val; leu; ile |
| Arg (R) | lys; gln; asn |
| Asn (N) | gin; his; lys; arg |
| Asp (D) | Glu |
| Cys (C) | Ser |
| Gln (Q) | Asn |
| Glu (E) | Asp |
| Gly (G) | Pro |
| His (H) | asn: gin: lys: arg |
| Ile (I) | leu; val; met; ala; phe: norleucine |
| Leu (L) | norleucine: ile: val; met; ala: phe |
| Lys (K) | arg: gin: asn |
| Met (M) | leu; phe; ile |
| Phe (F) | leu: val: ile; ala |
| Pro (P) | Gly |
| Ser (S) | Thr |
| Thr (T) | Ser |
| Trp (W) | Tyr |
| Tyr(Y) | Trp: phe: thr: ser |
| Val (V) | Ile; leu; met; phe; ala; norleucine |

### III). Extended Recombinant Polypeptides

In one aspect, the invention provides XTEN polypeptide compositions that are useful as fusion protein partner(s) to link to and/or incorporate within a FVIII polypeptide, resulting in a CFXTEN fusion protein. XTEN are generally polypeptides with non-naturally occurring, substantially non-repetitive sequences having a low degree of or no secondary or tertiary structure under physiologic conditions. In one aspect, XTEN typically has from about 36 to about 3000 amino acids, and of which the majority are small hydrophilic amino acids. As used herein, “XTEN” specifically excludes whole antibodies or antibody fragments (e.g. single-chain antibodies and Fc fragments). XTEN polypeptides have utility as a fusion protein partners in that they serve in various roles, conferring certain desirable pharmacokinetic, physicochemical and pharmaceutical properties when linked to a FVIII protein to a create a CFXTEN fusion protein. Such CFXTEN fusion protein compositions have enhanced properties compared to the corresponding FVIII not linked to XTEN, making them useful in the treatment of certain diseases, disorders or conditions related to FVIII deficiencies or bleeding disorders, as more fully described below.

The selection criteria for the XTEN to be fused to the FVIII proteins used to create the inventive fusion proteins compositions generally relate to attributes of physical/chemical properties and conformational structure of the XTEN that is, in turn, used to confer the enhanced pharmaceutical and pharmacokinetic properties to the fusion proteins compositions. The unstructured characteristic and physical/chemical properties of the XTEN result, at least, in part, from the overall amino acid composition, the non-repetitive design, and the length of the XTEN polypeptide. The properties of XTEN are not tied to absolute amino acid sequences as evidenced by the diversity of the exemplary sequences of Table 4 that, within varying ranges of length, possess similar properties. The XTEN of the present invention may exhibit one or more, or all of the following advantageous properties: unstructured conformation, conformational flexibility, enhanced aqueous solubility, high degree of protease resistance, low immunogenicity, low binding to mammalian receptors, a defined degree of charge, and increased hydrodynamic (or Stokes) radii; properties that can make them particularly useful as fusion protein partners. Non-limiting examples of the enhanced properties of the fusion proteins comprising FVIII fused to XTEN, compared to FVIII not linked to XTEN, include increases in the overall solubility and/or metabolic stability, reduced susceptibility to proteolysis, reduced immunogenicity, reduced rate of absorption when administered subcutaneously or intramuscularly, reduced binding to FVIII clearance receptors, enhanced interactions with substrate, and/or enhanced pharmacokinetic properties when administered to a subject. Enhanced pharmacokinetic properties of the CFXTEN compositions compared to FVIII not linked to XTEN include longer terminal half-life (e.g., two-fold, three-fold, four-fold or more), increased area under the curve (AUC) (e.g., 25%, 50%, 100% or more), lower volume of distribution, and enhanced absorption after subcutaneous or intramuscular injection (an advantage compared to commercially-available forms of FVIII that must be administered intravenously). In addition, it is specifically contemplated that the CFXTEN compositions comprising cleavage sequences (described more fully, below) permit sustained release of biologically active FVIII, such that the administered CFXTEN acts as a depot. It is specifically contemplated that the inventive CFXTEN fusion proteins can exhibit one or more or any combination of the improved properties disclosed herein. As a result of these enhanced properties, it is believed that CFXTEN compositions permit less frequent dosing compared to FVIII not linked to XTEN and administered at a comparable dose. Such CFXTEN fusion protein compositions have utility to treat certain factor VIII-related diseases, disorders or conditions, as described herein.

A variety of methods and assays are known in the art for determining the physical/chemical properties of proteins such as the CFXTEN compositions comprising XTEN. Such properties include but are not limited to secondary or tertiary structure, solubility, protein aggregation, melting properties, contamination and water content. Such methods include analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Arnau, et al., Prot Expr and Purif (2006) 48, 1-13.

The XTEN component(s) of the CFXTEN are designed to behave like denatured peptide sequences under physiological conditions, despite the extended length of the polymer. “Denatured” describes the state of a peptide in solution that is characterized by a large conformational freedom of the peptide backbone. Most peptides and proteins adopt a denatured conformation in the presence of high concentrations of denaturants or at elevated temperature. Peptides in denatured conformation have, for example, characteristic circular dichroism (CD) spectra and are characterized by a lack of long-range interactions as determined by NMR. “Denatured conformation” and “unstructured conformation” are used synonymously herein. In some embodiments, the invention provides XTEN sequences that, under physiologic conditions, are largely devoid of secondary structure. In other cases, the XTEN sequences are substantially devoid of secondary structure under physiologic conditions. “Largely devoid,” as used in this context, means that at least 50% of the XTEN amino acid residues of the XTEN sequence do not contribute to secondary structure as measured or determined by the means described herein. “Substantially devoid,” as used in this context, means that at least about 60%, or about 70%, or about 80%, or about 90%, or about 95%, or at least about 99% of the XTEN amino acid residues of the XTEN sequence do not contribute to secondary structure, as measured or determined by the methods described herein.

A variety of methods have been established in the art to discern the presence or absence of secondary and tertiary structures in a given polypeptide. In particular, secondary structure can be measured spectrophotometrically, e.g., by circular dichroism spectroscopy in the “far-UV” spectral region (190-250 nm). Secondary structure elements, such as alpha-helix and beta-sheet, each give rise to a characteristic shape and magnitude of CD spectra. Secondary structure can also be predicted for a polypeptide sequence via certain computer programs or algorithms, such as the well-known Chou-Fasman algorithm (Chou, P. Y., et al. (1974) Biochemistry, 13: 222-45) and the Garnier-Osguthorpe-Robson (“GOR”) algorithm (Garnier J, Gibrat J F, Robson B. (1996), GOR method for predicting protein secondary structure from amino acid sequence. Methods Enzymol 266:540-553), as described in US Patent Application Publication No. 20030228309A1. For a given sequence, the algorithms can predict whether there exists some or no secondary structure at all, expressed as the total and/or percentage of residues of the sequence that form, for example, alpha-helices or beta-sheets or the percentage of residues of the sequence predicted to result in random coil formation (which lacks secondary structure).

In one embodiment, the XTEN sequences used in the subject fusion protein compositions have an alpha-helix percentage ranging from 0% to less than about 5% as determined by the Chou-Fasman algorithm. In another embodiment, the XTEN sequences of the fusion protein compositions have a beta-sheet percentage ranging from 0% to less than about 5% as determined by the Chou-Fasman algorithm. In some embodiments, the XTEN sequences of the fusion protein compositions have an alpha-helix percentage ranging from 0% to less than about 5% and a beta-sheet percentage ranging from 0% to less than about 5% as determined by the Chou-Fasman algorithm. In some embodiments, the XTEN sequences of the fusion protein compositions have an alpha-helix percentage less than about 2% and a beta-sheet percentage less than about 2%. The XTEN sequences of the fusion protein compositions have a high degree of random coil percentage, as determined by the GOR algorithm. In some embodiments, an XTEN sequence have at least about 80%, at least about 90%, at least about 91%, at least about 92%, at least about 93%, at least about 94%, at least about 95%, at least about 96%, at least about 97%, at least about 98%, and most preferably at least about 99% random coil, as determined by the GOR algorithm. In some embodiments, the XTEN sequences of the fusion protein compositions have an alpha-helix percentage ranging from 0% to less than about 5% and a beta-sheet percentage ranging from 0% to less than about 5% as determined by the Chou-Fasman algorithm and at least about 90% random coil, as determined by the GOR algorithm. In other embodiments, the XTEN sequences of the fusion protein compositions have an alpha-helix percentage less than about 2% and a beta-sheet percentage less than about 2% at least about 90% random coil, as determined by the GOR algorithm.

### 1. Non-Repetitive Sequences

It is contemplated that the XTEN sequences of the CFXTEN embodiments are substantially non-repetitive. In general, repetitive amino acid sequences have a tendency to aggregate or form higher order structures, as exemplified by natural repetitive sequences such as collagens and leucine zippers. These repetitive amino acids may also tend to form contacts resulting in crystalline or pseudocrystalline structures. In contrast, the low tendency of non-repetitive sequences to aggregate enables the design of long-sequence XTENs with a relatively low frequency of charged amino acids that would otherwise be likely to aggregate if the sequences were repetitive. The non-repetitiveness of a subject XTEN can be observed by assessing one or more of the following features. In one embodiment, a “substantially non-repetitive” XTEN sequence has about 36, or at least 72, or at least 96, or at least 144, or at least 288, or at least 400, or at least 500, or at least 600, or at least 700, or at least 800, or at least 864, or at least 900, or at least 1000, or at least 2000, to about 3000 or more amino acid residues, or has a length ranging from about 36 to about 3000, about 100 to about 500, about 500 to about 1000, about 1000 to about 3000 amino acids and residues, in which no three contiguous amino acids in the sequence are identical amino acid types unless the amino acid is serine, in which case no more than three contiguous amino acids are serine residues. In another embodiment, as described more fully below, a “substantially non-repetitive” XTEN sequence comprises motifs of 9 to 14 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in any one motif is not repeated more than twice in the sequence motif.

The degree of repetitiveness of a polypeptide or a gene can be measured by computer programs or algorithms or by other means known in the art. According to the current invention, algorithms to be used in calculating the degree of repetitiveness of a particular polypeptide, such as an XTEN, are disclosed herein, and examples of sequences analyzed by algorithms are provided (see Examples, below). In one aspect, the repetitiveness of a polypeptide of a predetermined length can be calculated (hereinafter “subsequence score”) according to the formula given by Equation 1:

$$Subsequence score \frac{{\sum }_{i = 1}^{m} {Count}_{i}}{m} \qquad I$$

wherein: m=(amino acid length of polypeptide)−(amino acid length of subsequence)+1;
and Countᵢ=cumulative number of occurrences of each unique subsequence within sequenceᵢ

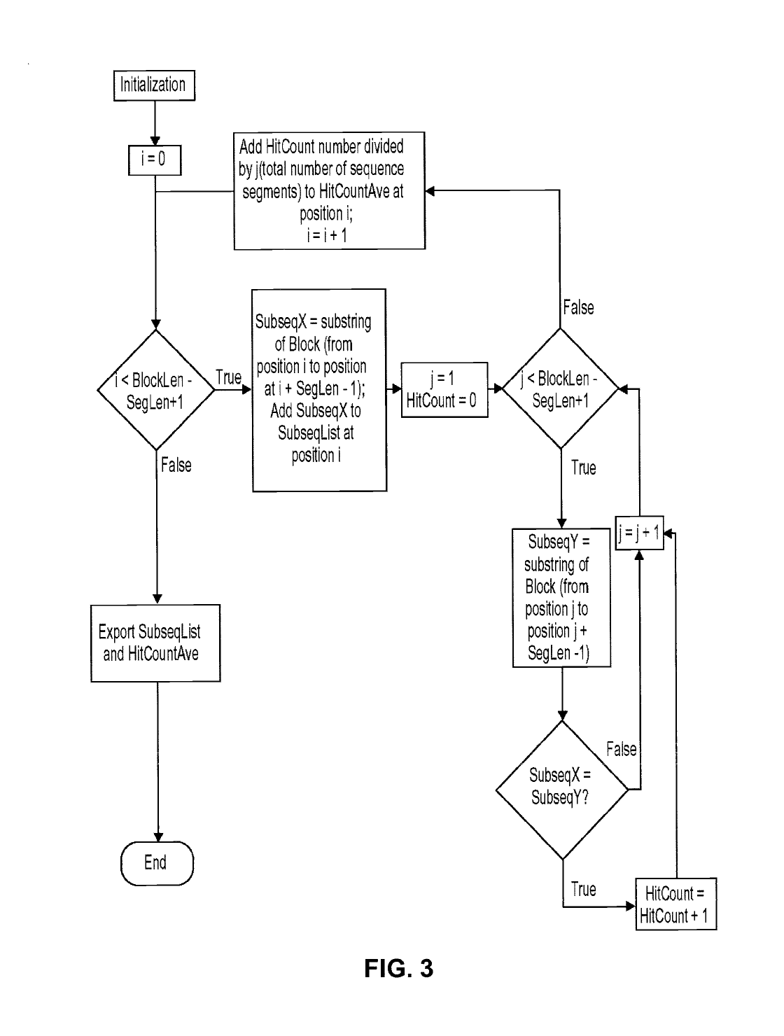

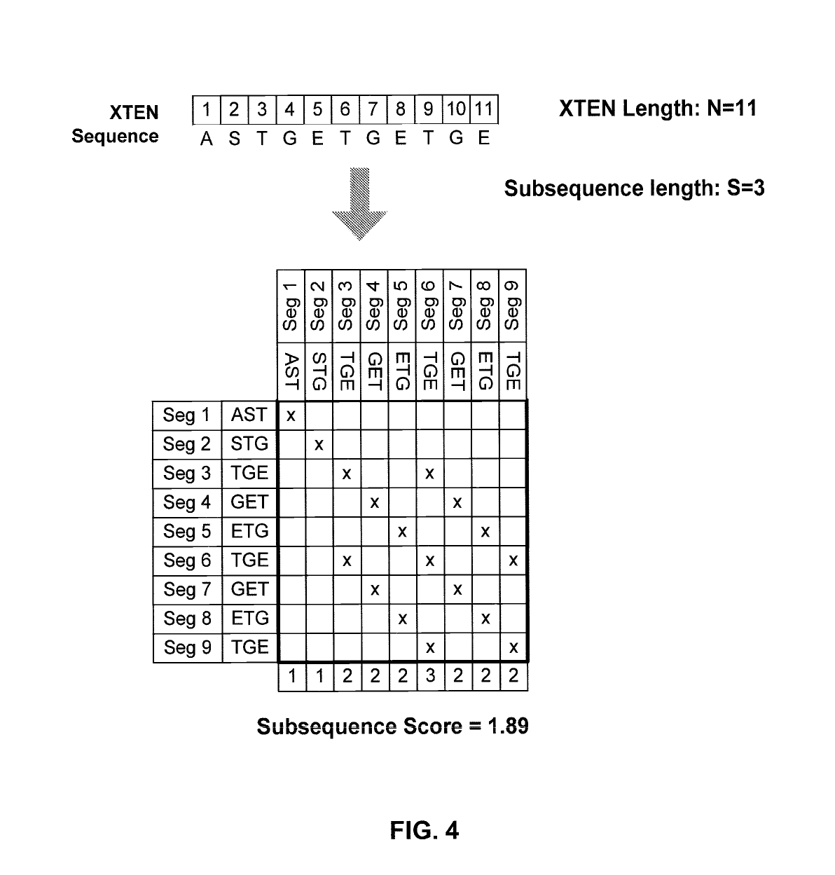

An algorithm termed “SegScore” was developed to apply the foregoing equation to quantitate repetitiveness of polypeptides, such as an XTEN, providing the subsequence score wherein sequences of a predetermined amino acid length “n” are analyzed for repetitiveness by determining the number of times (a “count”) a unique subsequence of length “s” appears in the set length, divided by the absolute number of subsequences within the predetermined length of the sequence. FIG. 3 depicts a logic flowchart of the SegScore algorithm, while FIG. 4 portrays a schematic of how a subsequence score is derived for a fictitious XTEN with 11 amino acids and a subsequence length of 3 amino acid residues. For example, a predetermined polypeptide length of 200 amino acid residues has 192 overlapping 9-amino acid subsequences and 198 3-mer subsequences, but the subsequence score of any given polypeptide will depend on the absolute number of unique subsequences and how frequently each unique subsequence (meaning a different amino acid sequence) appears in the predetermined length of the sequence.

In the context of the present invention, “subsequence score” means the sum of occurrences of each unique 3-mer frame across a 200 consecutive amino acid sequence of the polypeptide divided by the absolute number of unique 3-mer subsequences within the 200 amino acid sequence. Examples of such subsequence scores derived from the first 200 amino acids of repetitive and non-repetitive polypeptides are presented in Example 32. In one embodiment, the invention provides a CFXTEN comprising one XTEN in which the XTEN has a subsequence score less than 12, more preferably less than 10, more preferably less than 9, more preferably less than 8, more preferably less than 7, more preferably less than 6, and most preferably less than 5. In another embodiment, the invention provides CFXTEN comprising at least two to about six XTEN in which at least one XTEN has a subsequence score of less than 10, more preferably less than 9, more preferably less than 8, more preferably less than 7, more preferably less than 6, and most preferably less than 5. In the embodiments of the CFXTEN fusion protein compositions described herein, an XTEN component of a fusion protein with a subsequence score of 10 or less (i.e., 9, 8, 7, etc.) is also substantially non-repetitive.

It is believed that the non-repetitive characteristic of XTEN of the present invention together with the particular types of amino acids that predominate in the XTEN, rather than the absolute primary sequence, confers many of the enhanced physicochemical and biological properties of the CFXTEN fusion proteins. These enhanced properties include a higher degree of expression of the fusion protein in the host cell, greater genetic stability of the gene encoding XTEN, a greater degree of solubility, less tendency to aggregate, and enhanced pharmacokinetics of the resulting CFXTEN compared to fusion proteins comprising polypeptides having repetitive sequences. These enhanced properties permit more efficient manufacturing, lower cost of goods, and facilitate the formulation of XTEN-comprising pharmaceutical preparations containing extremely high protein concentrations, in some cases exceeding 100 mg/ml. Furthermore, the XTEN polypeptide sequences of the embodiments are designed to have a low degree of internal repetitiveness in order to reduce or substantially eliminate immunogenicity when administered to a mammal. Polypeptide sequences composed of short, repeated motifs largely limited to only three amino acids, such as glycine, serine and glutamate, may result in relatively high antibody titers when administered to a mammal despite the absence of predicted T-cell epitopes in these sequences. This may be caused by the repetitive nature of polypeptides, as it has been shown that immunogens with repeated epitopes, including protein aggregates, cross-linked immunogens, and repetitive carbohydrates are highly immunogenic and can, for example, result in the cross-linking of B-cell receptors causing B-cell activation. (Johansson, J., et al. (2007) Vaccine, 25:1676-82; Yankai, Z., et al. (2006) Biochem Biophys Res Commun, 345:1365-71; Hsu, C. T., et al. (2000) Cancer Res, 60:3701-5); Bachmann M F, et al. Eur J Immunol. (1995) 25(12):3445-3451).

### 2. Exemplary Sequence Motifs

The present invention encompasses XTEN used as fusion partners that comprise multiple units of shorter sequences, or motifs, in which the amino acid sequences of the motifs are non-repetitive. The non-repetitive property is met despite the use of a “building block” approach using a library of sequence motifs that are multimerized to create the XTEN sequences. Thus, while an XTEN sequence may consist of multiple units of as few as four different types of sequence motifs, because the motifs themselves generally consist of non-repetitive amino acid sequences, the overall XTEN sequence is designed to render the sequence substantially non-repetitive.

In one embodiment, an XTEN has a substantially non-repetitive sequence of greater than about 36 to about 1000, or about 100 to about 2000, or about 400 to about 3000 amino acid residues, or even longer wherein at least about 80%, or at least about 85%, or at least about 90%, or at least about 95%, or at least about 97%, or about 100% of the XTEN sequence consists of non-overlapping sequence motifs, and wherein each of the motifs has about 9 to 36 amino acid residues. In other embodiments, at least about 80%, or at least about 85%, or at least about 90%, or at least about 95%, or at least about 97%, or about 100% of the XTEN sequence consists of non-overlapping sequence motifs wherein each of the motifs has 9 to 14 amino acid residues. In still other embodiments, at least about 80%, or at least about 85%, or at least about 90%, or at least about 95%, or at least about 97%, or about 100% of the XTEN sequence consists of non-overlapping sequence motifs wherein each of the motifs has 12 amino acid residues. In these embodiments, it is preferred that the sequence motifs are composed of substantially (e.g., 90% or more) or exclusively small hydrophilic amino acids, such that the overall sequence has an unstructured, flexible characteristic. Examples of amino acids that are included in XTEN are, e.g., arginine, lysine, threonine, alanine, asparagine, glutamine, aspartate, glutamate, serine, and glycine. As a result of testing variables such as codon optimization, assembly polynucleotides encoding sequence motifs, expression of protein, charge distribution and solubility of expressed protein, and secondary and tertiary structure, it was discovered that XTEN compositions with the enhanced characteristics disclosed herein mainly include glycine (G), alanine (A), serine (S), threonine (T), glutamate (E) and proline (P) residues wherein the sequences are designed to be substantially non-repetitive. In one embodiment, XTEN sequences have predominately four to six types of amino acids selected from glycine (G), alanine (A), serine (5), threonine (T), glutamate (E) or proline (P) that are arranged in a substantially non-repetitive sequence that is greater than about 36 to about 1000, or about 100 to about 2000, or about 400 to about 3000 amino acid residues in length. In some embodiment, an XTEN sequence is made of 4, 5, or 6 types of amino acids selected from the group consisting of glycine (G), alanine (A), serine (S), threonine (T), glutamate (E) or proline (P). In some embodiments, XTEN have sequences of greater than about 36 to about 1000, or about 100 to about 2000, or about 400 to about 3000 amino acid residues wherein at least about 80% of the sequence consists of non-overlapping sequence motifs wherein each of the motifs has 9 to 36 amino acid residues and wherein at least 90%, or at least 91%, or at least 92%, or at least 93%, or at least 94%, or at least 95%, or at least 96%, or at least 97%, or 100% of each of the motifs consists of 4 to 6 types of amino acids selected from glycine (G), alanine (A), serine (S), threonine (T), glutamate (E) and proline (P), and wherein the content of any one amino acid type in the full-length XTEN does not exceed 30%. In other embodiments, at least about 90% of the XTEN sequence consists of non-overlapping sequence motifs wherein each of the motifs has 9 to 36 amino acid residues wherein the motifs consist of 4 to 6 types of amino acids selected from glycine (G), alanine (A), serine (S), threonine (T), glutamate (E) and proline (P), and wherein the content of any one amino acid type in the full-length XTEN does not exceed 40%, or about 30%, or about 25%. In other embodiments, at least about 90% of the XTEN sequence consists of non-overlapping sequence motifs wherein each of the motifs has 12 amino acid residues consisting of 4 to 6 types of amino acids selected from glycine (G), alanine (A), serine (S), threonine (T), glutamate (E) and proline (P), and wherein the content of any one amino acid type in the full-length XTEN does not exceed 40%, or 30%, or about 25%. In yet other embodiments, at least about 90%, or about 91%, or about 92%, or about 93%, or about 94%, or about 95%, or about 96%, or about 97%, or about 98%, or about 99%, to about 100% of the XTEN sequence consists of non-overlapping sequence motifs wherein each of the motifs has 12 amino acid residues consisting of glycine (G), alanine (A), serine (S), threonine (T), glutamate (E) and proline (P).

In still other embodiments, XTENs comprise substantially non-repetitive sequences of greater than about 36 to about 3000 amino acid residues wherein at least about 80%, or at least about 90%, or about 91%, or about 92%, or about 93%, or about 94%, or about 95%, or about 96%, or about 97%, or about 98%, or about 99% of the sequence consists of non-overlapping sequence motifs of 9 to 14 amino acid residues wherein the motifs consist of 4 to 6 types of amino acids selected from glycine (G), alanine (A), serine (S), threonine (T), glutamate (E) and proline (P), and wherein the sequence of any two contiguous amino acid residues in any one motif is not repeated more than twice in the sequence motif. In other embodiments, at least about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of four to six types of amino acids selected from glycine (G), alanine (A), serine (S), threonine (T), glutamate (E) and proline (P), and wherein the sequence of any two contiguous amino acid residues in any one sequence motif is not repeated more than twice in the sequence motif. In other embodiments, at least about 90%, or about 91%, or about 92%, or about 93%, or about 94%, or about 95%, or about 96%, or about 97%, or about 98%, or about 99% of an XTEN sequence consists of non-overlapping sequence motifs of 12 amino acid residues wherein the motifs consist of glycine (G), alanine (A), serine (S), threonine (T), glutamate (E) and proline (P), and wherein the sequence of any two contiguous amino acid residues in any one sequence motif is not repeated more than twice in the sequence motif. In yet other embodiments, XTENs consist of 12 amino acid sequence motifs wherein the amino acids are selected from glycine (G), alanine (A), serine (S), threonine (T), glutamate (E) and proline (P), and wherein the sequence of any two contiguous amino acid residues in any one sequence motif is not repeated more than twice in the sequence motif, and wherein the content of any one amino acid type in the full-length XTEN does not exceed 30%. The foregoing embodiments are examples of substantially non-repetitive XTEN sequences. Additional examples are detailed below.

In some embodiments, the invention provides CFXTEN compositions comprising one, or two, or three, or four, five, six or more non-repetitive XTEN sequence(s) of about 36 to about 1000 amino acid residues, or cumulatively about 100 to about 3000 amino acid residues wherein at least about 80%, or at least about 90%, or about 91%, or about 92%, or about 93%, or about 94%, or about 95%, or about 96%, or about 97%, or about 98%, or about 99% to about 100% of the sequence consists of multiple units of four or more non-overlapping sequence motifs selected from the amino acid sequences of Table 3, wherein the overall sequence remains substantially non-repetitive. In some embodiments, the XTEN comprises non-overlapping sequence motifs in which about 80%, or at least about 85%, or at least about 90%, or about 91%, or about 92%, or about 93%, or about 94%, or about 95%, or about 96%, or about 97%, or about 98%, or about 99% or about 100% of the sequence consists of multiple units of non-overlapping sequences selected from a single motif family selected from Table 3, resulting in a family sequence. As used herein, “family” means that the XTEN has motifs selected only from a single motif category from Table 3; i.e., AD, AE, AF, AG, AM, AQ, BC, or BD XTEN, and that any other amino acids in the XTEN not from a family motif are selected to achieve a needed property, such as to permit incorporation of a restriction site by the encoding nucleotides, incorporation of a cleavage sequence, or to achieve a better linkage to a FVIII coagulation factor component of the CFXTEN. In some embodiments of XTEN families, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology described above. In other embodiments, the XTEN comprises multiple units of motif sequences from two or more of the motif families of Table 3. These sequences can be selected to achieve desired physical/chemical characteristics, including such properties as net charge, lack of secondary structure, or lack of repetitiveness that are conferred by the amino acid composition of the motifs, described more fully below. In the embodiments hereinabove described in this paragraph, the motifs incorporated into the XTEN can be selected and assembled using the methods described herein to achieve an XTEN of about 36 to about 3000 amino acid residues.

TABLE 3
XTEN Sequence Motifs of 12 Amino Acids
and Motif Families

| Motif Family* | MOTIF SEQUENCE |
|---|---|
| AD | GESPGGSSGSES |
| AD | GSEGSSGPGESS |
| AD | GSSESGSSEGGP |
| AD | GSGGEPSESGSS |
| AE, AM | GSPAGSPTSTEE |
| AE, AM, AQ | GSEPATSGSETP |
| AE, AM, AQ | GTSESATPESGP |
| AE, AM, AQ | GTSTEPSEGSAP |
| AF, AM | GSTSESPSGTAP |
| AF, AM | GTSTPESGSASP |
| AF, AM | GTSPSGESSTAP |
| AF, AM | GSTSSTAESPGP |
| AG, AM | GTPGSGTASSSP |
| AG, AM | GSSTPSGATGSP |
| AG, AM | GSSPSASTGTGP |
| AG, AM | GASPGTSSTGSP |
| AQ | GEPAGSPTSTSE |
| AQ | GTGEPSSTPASE |
| AQ | GSGPSTESAPTE |
| AQ | GSETPSGPSETA |
| AQ | GPSETSTSEPGA |
| AQ | GSPSEPTEGTSA |
| BC | GSGASEPTSTEP |
| BC | GSEPATSGTEPS |
| BC | GTSEPSTSEPGA |
| BC | GTSTEPSEPGSA |
| BD | GSTAGSETSTEA |
| BD | GSETATSGSETA |
| BD | GTSESATSESGA |
| BD | GTSTEASEGSAS |

*Denotes individual motif sequences that, when used together in various permutations, results in a “family sequence”

In some embodiments of XTEN families, an XTEN sequence comprises multiple units of non-overlapping sequence motifs of the AD motif family, the AE motif family, or the AF motif family, or the AG motif family, or the AM motif family, or the AQ motif family, or the BC family, or the BD family, with the resulting XTEN exhibiting the range of homology described above. In other embodiments, the XTEN comprises multiple units of motif sequences from two or more of the motif families of Table 3, selected to achieve desired physicochemical characteristics, including such properties as net charge, lack of secondary structure, or lack of repetitiveness that may be conferred by the amino acid composition of the motifs, described more fully below. In the embodiments hereinabove described in this paragraph, the motifs incorporated into the XTEN can be selected and assembled using the methods described herein to achieve an XTEN of about 36 to about 3000 amino acid residues. Non-limiting examples of XTEN family sequences are presented in Table 4.

TABLE 4
XTEN Polypeptides

| XTEN | |
|---|---|
| Name | Amino Acid Sequence |
| AE42_1 | TEPSEGSAPGSPAGSPTSTEEGTSESATPESGPGSEPATSGS |
| AE42_2 | PAGSPTSTEEGTSTEPSEGSAPGTSESATPESGPGSEPATSG |
| AE42_3 | SEPATSGSETPGTSESATPESGPGTSTEPSEGSAPGSPAGSP |
| AE42_4 | GAPGSPAGSPTSTEEGTSESATPESGPGSEPATSGSETPASS |
| AG42_1 | GAPSPSASTGTGPGTPGSGTASSSPGSSTPSGATGSPGPSGP |
| AG42_2 | GPGTPGSGTASSSPGSSTPSGATGSPGSSPSASTGTGPGASP |
| AG42_3 | SPSASTGTGPGASPGTSSTGSPGTPGSGTASSSPGSSTPSGA |
| AG42_4 | SASTGTGPGASPGTSSTGSPGTPGSGTASSSPGSSTPSGATG |
| AE48 | MAEPAGSPTSTEEGTPGSGTASSSPGSSTPSGATGSPGASPGTSSTGS |
| AM48 | MAEPAGSPTSTEEGASPGTSSTGSPGSSTPSGATGSPGSSTPSGATGS |
| AE144 | GSEPATSGSETPGTSESATPESGPGSEPATSGSETPGSPAGSPTSTEEGTSTEPSEGSAPGSEPATSG |
| | SETPGSEPATSGSETPGSEPATSGSETPGTSTEPSEGSAPGTSESATPESGPGSEPATSGSETPGTST |
| | EPSEGSAP |
| AF144 | GTSTPESGSASPGTSPSGESSTAPGTSPSGESSTAPGSTSSTAESPGPGSTSESPSGTAPGSTSSTAE |
| | SPGPGTSPSGESSTAPGTSTPESGSASPGSTSSTAESPGPGTSPSGESSTAPGTSPSGESSTAPGTSP |
| | SGESSTAP |
| AG144_1 | PGSSPSASTGTGPGSSPSASTGTGPGTPGSGTASSSPGSSTPSGATGSPGSSPSASTGTGPGASPGT |
| | SSTGSPGTPGSGTASSSPGSSTPSGATGSPGTPGSGTASSSPGASPGTSSTGSPGASPGTSSTGSPG |
| | TPGSGTASSS |
| AG144_2 | SGTASSSPGSSTPSGATGSPGTPGSGTASSSPGSSTPSGATGSPGSSTPSGATGSPGSSPSASTGTG |
| | PGSSPSASTGTGPGASPGTSSTGSPGTPGSGTASSSPGSSTPSGATGSPGSSPSASTGTGPGSSPSAS |
| | TGTGPGASP |
| AG144_3 | GTPGSGTASSSPGSSTPSGATGSPGSSTPSGATGSPGSSPSASTGTGPGSSPSASTGTGPGASPGTS |
| | STGSPGTPGSGTASSSPGSSTPSGATGSPGSSPSASTGTGPGSSPSASTGTGPGASPGTSSTGSPGA |
| | SPGTSSTGSP |
| AG144_4 | GTPGSGTASSSPGSSTPSGATGSPGSSPSASTGTGPGSSPSASTGTGPGASPGTSSTGSPGASPGTS |
| | STGSPGSSTPSGATGSPGSSPSASTGTGPGASPGTSSTGSPGSSPSASTGTGPGTPGSGTASSSPGS |
| | STPSGATGSP |
| AE288 | GTSESATPESGPGSEPATSGSETPGTSESATPESGPGSEPATSGSETPGTSESATPESGPGTSTEPSE |
| | GSAPGSPAGSPTSTEEGTSESATPESGPGSEPATSGSETPGTSESATPESGPGSPAGSPTSTEEGSP |
| | AGSPTSTEEGTSTEPSEGSAPGTSESATPESGPGTSESATPESGPGTSESATPESGPGSEPATSGSET |
| | PGSEPATSGSETPGSPAGSPTSTEEGTSTEPSEGSAPGTSTEPSEGSAPGSEPATSGSETPGTSESAT |
| | PESGPGTSTEPSEGSAP |
| AG288_1 | ASSSPGASPGTSSTGSPGASPGTSSTGSPGASPGTSSTGSPGASPGTSSTGSPGTPGSGTASSSPGSS |
| | TPSGATGSPGTPGSGTASSSPGSSTPSGATGSPGTPGSGTASSSPGSSTPSGATGSPGSSTPSGATG |
| | SPGSSPSASTGTGPGSSPSASTGTGPGASPGTSSTGSPGTPGSGTASSSPGSSTPSGATGSPGSSPSA |
| | STGTGPGSSPSASTGTGPGASPGTSSTGSPGASPGTSSTGSPGSSTPSGATGSPGSSPSASTGTGPG |
| | ASPGTSSTGSPGSSPSASTGTGPGTPGSGTASSSPGSSTPSGATGS |
| AG288_2 | PGASPGTSSTGSPGASPGTSSTGSPGTPGSGTASSSPGSSTPSGATGSPGTPGSGTASSSPGSSTPS |
| | GATGSPGTPGSGTASSSPGSSTPSGATGSPGSSTPSGATGSPGSSPSASTGTGPGSSPSASTGTGPG |
| | ASPGTSSTGSPGTPGSGTASSSPGSSTPSGATGSPGSSPSASTGTGPGSSPSASTGTGPGASPGTSS |
| | TGSPGASPGTSSTGSPGSSTPSGATGSPGSSPSASTGTGPGASPGTSSTGSPGSSPSASTGTGPGTP |
| | GSGTASSSPGSSTPSGATGS |
| AG288_3 | GSSPSASTGTGPGSSPSASTGTGPGTPGSGTASSSPGSSTPSGATGSPGSSPSASTGTGPGASPGTS |
| | STGSPGTPGSGTASSSPGSSTPSGATGSPGTPGSGTASSSPGASPGTSSTGSPGASPGTSSTGSPGT |
| | PGSGTASSSPGSSTPSGATGSPGASPGTSSTGSPGTPGSGTASSSPGSSTPSGATGSPGSSPSASTG |
| | TGPGSSPSASTGTGPGSSTPSGATGSPGSSTPSGATGSPGASPGTSSTGSPGASPGTSSTGSPGASP |
| | GTSSTGSPGTPGSGTASSSP |
| AF504 | GASPGTSSTGSPGSSPSASTGTGPGSSPSASTGTGPGTPGSGTASSSPGSSTPSGATGSPGSXPSAS |
| | TGTGPGASPGTSSTGSPGTPGSGTASSSPGSSTPSGATGSPGTPGSGTASSSPGASPGTSSTGSPGA |
| | SPGTSSTGSPGTPGSGTASSSPGSSTPSGATGSPGASPGTSSTGSPGTPGSGTASSSPGSSTPSGAT |
| | GSPGSXPSASTGTGPGSSPSASTGTGPGSSTPSGATGSPGSSTPSGATGSPGASPGTSSTGSPGASP |
| | GTSSTGSPGASPGTSSTGSPGTPGSGTASSSPGASPGTSSTGSPGASPGTSSTGSPGASPGTSSTGS |
| | PGSSPSASTGTGPGTPGSGTASSSPGASPGTSSTGSPGASPGTSSTGSPGASPGTSSTGSPGSSTPS |
| | GATGSPGSSTPSGATGSPGASPGTSSTGSPGTPGSGTASSSPGSSTPSGATGSPGSSTPSGATGSPG |
| | SSTPSGATGSPGSSPSASTGTGPGASPGTSSTGSP |
| AF540 | GSTSSTAESPGPGSTSSTAESPGPGSTSESPSGTAPGSTSSTAESPGPGSTSSTAESPGPGTSTPESG |
| | SASPGSTSESPSGTAPGTSPSGESSTAPGSTSESPSGTAPGSTSESPSGTAPGTSPSGESSTAPGSTS |
| | ESPSGTAPGSTSESPSGTAPGTSPSGESSTAPGSTSESPSGTAPGSTSESPSGTAPGSTSESPSGTAP |
| | GTSTPESGSASPGSTSESPSGTAPGTSTPESGSASPGSTSSTAESPGPGSTSSTAESPGPGTSTPESG |
| | SASPGTSTPESGSASPGSTSESPSGTAPGTSTPESGSASPGTSTPESGSASPGSTSESPSGTAPGSTS |
| | ESPSGTAPGSTSESPSGTAPGSTSSTAESPGPGTSTPESGSASPGTSTPESGSASPGSTSESPSGTAP |
| | GSTSESPSGTAPGTSTPESGSASPGSTSESPSGTAPGSTSESPSGTAPGTSTPESGSASPGTSPSGES |
| | STAPGSTSSTAESPGPGTSPSGESSTAPGSTSSTAESPGPGTSTPESGSASPGSTSESPSGTAP |
| AD576 | GSSESGSSEGGPGSGGEPSESGSSGSSESGSSEGGPGSSESGSSEGGPGSSESGSSEGGPGSSESGS |
| | SEGGPGSSESGSSEGGPGESPGGSSGSESGSEGSSGPGESSGSSESGSSEGGPGSSESGSSEGGPGS |
| | SESGSSEGGPGSGGEPSESGSSGESPGGSSGSESGESPGGSSGSESGSGGEPSESGSSGSSESGSSE |
| | GGPGSGGEPSESGSSGSGGEPSESGSSGSEGSSGPGESSGESPGGSSGSESGSGGEPSESGSSGSGG |
| | EPSESGSSGSGGEPSESGSSGSSESGSSEGGPGESPGGSSGSESGESPGGSSGSESGESPGGSSGSES |
| | GESPGGSSGSESGESPGGSSGSESGSSESGSSEGGPGSGGEPSESGSSGSEGSSGPGESSGSSESGS |
| | SEGGPGSGGEPSESGSSGSSESGSSEGGPGSGGEPSESGSSGESPGGSSGSESGESPGGSSGSESGS |
| | SESGSSEGGPGSGGEPSESGSSGSSESGSSEGGPGSGGEPSESGSSGSGGEPSESGSSGESPGGSSG |
| | SESGSEGSSGPGESSGSSESGSSEGGPGSEGSSGPGESS |
| AE576 | GSPAGSPTSTEEGTSESATPESGPGTSTEPSEGSAPGSPAGSPTSTEEGTSTEPSEGSAPGTSTEPSE |
| | GSAPGTSESATPESGPGSEPATSGSETPGSEPATSGSETPGSPAGSPTSTEEGTSESATPESGPGTST |
| | EPSEGSAPGTSTEPSEGSAPGSPAGSPTSTEEGTSTEPSEGSAPGTSTEPSEGSAPGTSESATPESGP |
| | GTSTEPSEGSAPGTSESATPESGPGSEPATSGSETPGTSTEPSEGSAPGTSTEPSEGSAPGTSESATP |
| | ESGPGTSESATPESGPGSPAGSPTSTEEGTSESATPESGPGSEPATSGSETPGTSESATPESGPGTST |
| | EPSEGSAPGTSTEPSEGSAPGTSTEPSEGSAPGTSTEPSEGSAPGTSTEPSEGSAPGTSTEPSEGSAP |
| | GSPAGSPTSTEEGTSTEPSEGSAPGTSESATPESGPGSEPATSGSETPGTSESATPESGPGSEPATSG |
| | SETPGTSESATPESGPGTSTEPSEGSAPGTSESATPESGPGSPAGSPTSTEEGSPAGSPTSTEEGSPA |
| | GSPTSTEEGTSESATPESGPGTSTEPSEGSAP |
| AF576 | GSTSSTAESPGPGSTSSTAESPGPGSTSESPSGTAPGSTSSTAESPGPGSTSSTAESPGPGTSTPESG |
| | SASPGSTSESPSGTAPGTSPSGESSTAPGSTSESPSGTAPGSTSESPSGTAPGTSPSGESSTAPGSTS |
| | ESPSGTAPGSTSESPSGTAPGTSPSGESSTAPGSTSESPSGTAPGSTSESPSGTAPGSTSESPSGTAP |
| | GTSTPESGSASPGSTSESPSGTAPGTSTPESGSASPGSTSSTAESPGPGSTSSTAESPGPGTSTPESG |
| | SASPGTSTPESGSASPGSTSESPSGTAPGTSTPESGSASPGTSTPESGSASPGSTSESPSGTAPGSTS |
| | ESPSGTAPGSTSESPSGTAPGSTSSTAESPGPGTSTPESGSASPGTSTPESGSASPGSTSESPSGTAP |
| | GSTSESPSGTAPGTSTPESGSASPGSTSESPSGTAPGSTSESPSGTAPGTSTPESGSASPGTSPSGES |
| | STAPGSTSSTAESPGPGTSPSGESSTAPGSTSSTAESPGPGTSTPESGSASPGSTSESPSGTAPGSTS |
| | STAESPGPGTSTPESGSASPGTSTPESGSASP |
| AG576 | PGTPGSGTASSSPGSSTPSGATGSPGSSPSASTGTGPGSSPSASTGTGPGSSTPSGATGSPGSSTPS |
| | GATGSPGASPGTSSTGSPGASPGTSSTGSPGASPGTSSTGSPGTPGSGTASSSPGASPGTSSTGSPG |
| | ASPGTSSTGSPGASPGTSSTGSPGSSPSASTGTGPGTPGSGTASSSPGASPGTSSTGSPGASPGTSS |
| | TGSPGASPGTSSTGSPGSSTPSGATGSPGSSTPSGATGSPGASPGTSSTGSPGTPGSGTASSSPGSS |
| | TPSGATGSPGSSTPSGATGSPGSSTPSGATGSPGSSPSASTGTGPGASPGTSSTGSPGASPGTSSTG |
| | SPGTPGSGTASSSPGASPGTSSTGSPGASPGTSSTGSPGASPGTSSTGSPGASPGTSSTGSPGTPGS |
| | GTASSSPGSSTPSGATGSPGTPGSGTASSSPGSSTPSGATGSPGTPGSGTASSSPGSSTPSGATGSP |
| | GSSTPSGATGSPGSSPSASTGTGPGSSPSASTGTGPGASPGTSSTGSPGTPGSGTASSSPGSSTPSG |
| | ATGSPGSSPSASTGTGPGSSPSASTGTGPGASPGTSSTGS |
| AE624 | MAEPAGSPTSTEEGTPGSGTASSSPGSSTPSGATGSPGASPGTSSTGSPGSPAGSPTSTEEGTSESA |
| | TPESGPGTSTEPSEGSAPGSPAGSPTSTEEGTSTEPSEGSAPGTSTEPSEGSAPGTSESATPESGPGS |
| | EPATSGSETPGSEPATSGSETPGSPAGSPTSTEEGTSESATPESGPGTSTEPSEGSAPGTSTEPSEGS |
| | APGSPAGSPTSTEEGTSTEPSEGSAPGTSTEPSEGSAPGTSESATPESGPGTSTEPSEGSAPGTSESA |
| | TPESGPGSEPATSGSETPGTSTEPSEGSAPGTSTEPSEGSAPGTSESATPESGPGTSESATPESGPGS |
| | PAGSPTSTEEGTSESATPESGPGSEPATSGSETPGTSESATPESGPGTSTEPSEGSAPGTSTEPSEGS |
| | APGTSTEPSEGSAPGTSTEPSEGSAPGTSTEPSEGSAPGTSTEPSEGSAPGSPAGSPTSTEEGTSTEP |
| | SEGSAPGTSESATPESGPGSEPATSGSETPGTSESATPESGPGSEPATSGSETPGTSESATPESGPGT |
| | STEPSEGSAPGTSESATPESGPGSPAGSPTSTEEGSPAGSPTSTEEGSPAGSPTSTEEGTSESATPES |
| | GPGTSTEPSEGSAP |
| AD836 | GSSESGSSEGGPGSSESGSSEGGPGESPGGSSGSESGSGGEPSESGSSGESPGGSSGSESGESPGGS |
| | SGSESGSSESGSSEGGPGSSESGSSEGGPGSSESGSSEGGPGESPGGSSGSESGESPGGSSGSESGE |
| | SPGGSSGSESGSSESGSSEGGPGSSESGSSEGGPGSSESGSSEGGPGSSESGSSEGGPGSSESGSSE |
| | GGPGSSESGSSEGGPGSGGEPSESGSSGESPGGSSGSESGESPGGSSGSESGSGGEPSESGSSGSEG |
| | SSGPGESSGSSESGSSEGGPGSGGEPSESGSSGSEGSSGPGESSGSSESGSSEGGPGSGGEPSESGS |
| | SGESPGGSSGSESGSGGEPSESGSSGSGGEPSESGSSGSSESGSSEGGPGSGGEPSESGSSGSGGEP |
| | SESGSSGSEGSSGPGESSGESPGGSSGSESGSEGSSGPGESSGSEGSSGPGESSGSGGEPSESGSSG |
| | SSESGSSEGGPGSSESGSSEGGPGESPGGSSGSESGSGGEPSESGSSGSEGSSGPGESSGESPGGSS |
| | GSESGSEGSSGPGSSESGSSEGGPGSGGEPSESGSSGSEGSSGPGESSGSEGSSGPGESSGSEGSSG |
| | PGESSGSGGEPSESGSSGSGGEPSESGSSGESPGGSSGSESGESPGGSSGSESGSGGEPSESGSSGS |
| | EGSSGPGESSGESPGGSSGSESGSSESGSSEGGPGSSESGSSEGGPGSSESGSSEGGPGSGGEPSES |
| | GSSGSSESGSSEGGPGESPGGSSGSESGSGGEPSESGSSGSSESGSSEGGPGESPGGSSGSESGSGG |
| | EPSESGSSGESPGGSSGSESGSGGEPSESGSS |
| AE864 | GSPAGSPTSTEEGTSESATPESGPGTSTEPSEGSAPGSPAGSPTSTEEGTSTEPSEGSAPGTSTEPSE |
| | GSAPGTSESATPESGPGSEPATSGSETPGSEPATSGSETPGSPAGSPTSTEEGTSESATPESGPGTST |
| | EPSEGSAPGTSTEPSEGSAPGSPAGSPTSTEEGTSTEPSEGSAPGTSTEPSEGSAPGTSESATPESGP |
| | GTSTEPSEGSAPGTSESATPESGPGSEPATSGSETPGTSTEPSEGSAPGTSTEPSEGSAPGTSESATP |
| | ESGPGTSESATPESGPGSPAGSPTSTEEGTSESATPESGPGSEPATSGSETPGTSESATPESGPGTST |
| | EPSEGSAPGTSTEPSEGSAPGTSTEPSEGSAPGTSTEPSEGSAPGTSTEPSEGSAPGTSTEPSEGSAP |
| | GSPAGSPTSTEEGTSTEPSEGSAPGTSESATPESGPGSEPATSGSETPGTSESATPESGPGSEPATSG |
| | SETPGTSESATPESGPGTSTEPSEGSAPGTSESATPESGPGSPAGSPTSTEEGSPAGSPTSTEEGSPA |
| | GSPTSTEEGTSESATPESGPGTSTEPSEGSAPGTSESATPESGPGSEPATSGSETPGTSESATPESGP |
| | GSEPATSGSETPGTSESATPESGPGTSTEPSEGSAPGSPAGSPTSTEEGTSESATPESGPGSEPATSG |
| | SETPGTSESATPESGPGSPAGSPTSTEEGSPAGSPTSTEEGTSTEPSEGSAPGTSESATPESGPGTSE |
| | SATPESGPGTSESATPESGPGSEPATSGSETPGSEPATSGSETPGSPAGSPTSTEEGTSTEPSEGSAP |
| | GTSTEPSEGSAPGSEPATSGSETPGTSESATPESGPGTSTEPSEGSAP |
| AF864 | GSTSESPSGTAPGTSPSGESSTAPGSTSESPSGTAPGSTSESPSGTAPGTSTPESGSASPGTSTPESG |
| | SASPGSTSESPSGTAPGSTSESPSGTAPGTSPSGESSTAPGSTSESPSGTAPGTSPSGESSTAPGTSP |
| | SGESSTAPGSTSSTAESPGPGTSPSGESSTAPGTSPSGESSTAPGSTSSTAESPGPGTSTPESGSASP |
| | GTSTPESGSASPGSTSESPSGTAPGSTSESPSGTAPGTSTPESGSASPGSTSSTAESPGPGTSTPESG |
| | SASPGSTSESPSGTAPGTSPSGESSTAPGSTSSTAESPGPGTSPSGESSTAPGTSTPESGSASPGSTS |
| | STAESPGPGSTSSTAESPGPGSTSSTAESPGPGSTSSTAESPGPGTSPSGESSTAPGSTSESPSGTAP |
| | GSTSESPSGTAPGTSTPESGPXXXGASASGAPSTXXXXSESPSGTAPGSTSESPSGTAPGSTSESPS |
| | GTAPGSTSESPSGTAPGSTSESPSGTAPGSTSESPSGTAPGTSTPESGSASPGTSPSGESSTAPGTSP |
| | SGESSTAPGSTSSTAESPGPGTSPSGESSTAPGTSTPESGSASPGSTSESPSGTAPGSTSESPSGTAP |
| | GTSPSGESSTAPGSTSESPSGTAPGTSTPESGSASPGTSTPESGSASPGSTSESPSGTAPGTSTPESG |
| | SASPGSTSSTAESPGPGSTSESPSGTAPGSTSESPSGTAPGTSPSGESSTAPGSTSSTAESPGPGTSP |
| | SGESSTAPGTSTPESGSASPGTSPSGESSTAPGTSPSGESSTAPGTSPSGESSTAPGSTSSTAESPGP |
| | GSTSSTAESPGPGTSPSGESSTAPGSSPSASTGTGPGSSTPSGATGSPGSSTPSGATGSP |
| AG864 | GASPGTSSTGSPGSSPSASTGTGPGSSPSASTGTGPGTPGSGTASSSPGSSTPSGATGSPGSSPSAS |
| | TGTGPGASPGTSSTGSPGTPGSGTASSSPGSSTPSGATGSPGTPGSGTASSSPGASPGTSSTGSPGA |
| | SPGTSSTGSPGTPGSGTASSSPGSSTPSGATGSPGASPGTSSTGSPGTPGSGTASSSPGSSTPSGAT |
| | GSPGSSPSASTGTGPGSSPSASTGTGPGSSTPSGATGSPGSSTPSGATGSPGASPGTSSTGSPGASP |
| | GTSSTGSPGASPGTSSTGSPGTPGSGTASSSPGASPGTSSTGSPGASPGTSSTGSPGASPGTSSTGS |
| | PGSSPSASTGTGPGTPGSGTASSSPGASPGTSSTGSPGASPGTSSTGSPGASPGTSSTGSPGSSTPS |
| | GATGSPGSSTPSGATGSPGASPGTSSTGSPGTPGSGTASSSPGSSTPSGATGSPGSSTPSGATGSPG |
| | SSTPSGATGSPGSSPSASTGTGPGASPGTSSTGSPGASPGTSSTGSPGTPGSGTASSSPGASPGTSS |
| | TGSPGASPGTSSTGSPGASPGTSSTGSPGASPGTSSTGSPGTPGSGTASSSPGSSTPSGATGSPGTP |
| | GSGTASSSPGSSTPSGATGSPGTPGSGTASSSPGSSTPSGATGSPGSSTPSGATGSPGSSPSASTGT |
| | GPGSSPSASTGTGPGASPGTSSTGSPGTPGSGTASSSPGSSTPSGATGSPGSSPSASTGTGPGSSPS |
| | ASTGTGPGASPGTSSTGSPGASPGTSSTGSPGSSTPSGATGSPGSSPSASTGTGPGASPGTSSTGSP |
| | GSSPSASTGTGPGTPGSGTASSSPGSSTPSGATGSPGSSTPSGATGSPGASPGTSSTGSP |
| AM875 | GTSTEPSEGSAPGSEPATSGSETPGSPAGSPTSTEEGSTSSTAESPGPGTSTPESGSASPGSTSESPS |
| | GTAPGSTSESPSGTAPGTSTPESGSASPGTSTPESGSASPGSEPATSGSETPGTSESATPESGPGSPA |
| | GSPTSTEEGTSTEPSEGSAPGTSESATPESGPGTSTEPSEGSAPGTSTEPSEGSAPGSPAGSPTSTEE |
| | GTSTEPSEGSAPGTSTEPSEGSAPGTSESATPESGPGTSESATPESGPGTSTEPSEGSAPGTSTEPSE |
| | GSAPGTSESATPESGPGTSTEPSEGSAPGSEPATSGSETPGSPAGSPTSTEEGSSTPSGATGSPGTP |
| | GSGTASSSPGSSTPSGATGSPGTSTEPSEGSAPGTSTEPSEGSAPGSEPATSGSETPGSPAGSPTSTE |
| | EGSPAGSPTSTEEGTSTEPSEGSAPGASASGAPSTGGTSESATPESGPGSPAGSPTSTEEGSPAGSP |
| | TSTEEGSTSSTAESPGPGSTSESPSGTAPGTSPSGESSTAPGTPGSGTASSSPGSSTPSGATGSPGSS |
| | PSASTGTGPGSEPATSGSETPGTSESATPESGPGSEPATSGSETPGSTSSTAESPGPGSTSSTAESPG |
| | PGTSPSGESSTAPGSEPATSGSETPGSEPATSGSETPGTSTEPSEGSAPGSTSSTAESPGPGTSTPES |
| | GSASPGSTSESPSGTAPGTSTEPSEGSAPGTSTEPSEGSAPGTSTEPSEGSAPGSSTPSGATGSPGSS |
| | PSASTGTGPGASPGTSSTGSPGSEPATSGSETPGTSESATPESGPGSPAGSPTSTEEGSSTPSGATG |
| | SPGSSPSASTGTGPGASPGTSSTGSPGTSESATPESGPGTSTEPSEGSAPGTSTEPSEGSAP |
| AE912 | MAEPAGSPTSTEEGTPGSGTASSSPGSSTPSGATGSPGASPGTSSTGSPGSPAGSPTSTEEGTSESA |
| | TPESGPGTSTEPSEGSAPGSPAGSPTSTEEGTSTEPSEGSAPGTSTEPSEGSAPGTSESATPESGPGS |
| | EPATSGSETPGSEPATSGSETPGSPAGSPTSTEEGTSESATPESGPGTSTEPSEGSAPGTSTEPSEGS |
| | APGSPAGSPTSTEEGTSTEPSEGSAPGTSTEPSEGSAPGTSESATPESGPGTSTEPSEGSAPGTSESA |
| | TPESGPGSEPATSGSETPGTSTEPSEGSAPGTSTEPSEGSAPGTSESATPESGPGTSESATPESGPGS |
| | PAGSPTSTEEGTSESATPESGPGSEPATSGSETPGTSESATPESGPGTSTEPSEGSAPGTSTEPSEGS |
| | APGTSTEPSEGSAPGTSTEPSEGSAPGTSTEPSEGSAPGTSTEPSEGSAPGSPAGSPTSTEEGTSTEP |
| | SEGSAPGTSESATPESGPGSEPATSGSETPGTSESATPESGPGSEPATSGSETPGTSESATPESGPGT |
| | STEPSEGSAPGTSESATPESGPGSPAGSPTSTEEGSPAGSPTSTEEGSPAGSPTSTEEGTSESATPES |
| | GPGTSTEPSEGSAPGTSESATPESGPGSEPATSGSETPGTSESATPESGPGSEPATSGSETPGTSESA |
| | TPESGPGTSTEPSEGSAPGSPAGSPTSTEEGTSESATPESGPGSEPATSGSETPGTSESATPESGPGS |
| | PAGSPTSTEEGSPAGSPTSTEEGTSTEPSEGSAPGTSESATPESGPGTSESATPESGPGTSESATPES |
| | GPGSEPATSGSETPGSEPATSGSETPGSPAGSPTSTEEGTSTEPSEGSAPGTSTEPSEGSAPGSEPAT |
| | SGSETPGTSESATPESGPGTSTEPSEGSAP |
| AM923 | MAEPAGSPTSTEEGASPGTSSTGSPGSSTPSGATGSPGSSTPSGATGSPGTSTEPSEGSAPGSEPAT |
| | SGSETPGSPAGSPTSTEEGSTSSTAESPGPGTSTPESGSASPGSTSESPSGTAPGSTSESPSGTAPGT |
| | STPESGSASPGTSTPESGSASPGSEPATSGSETPGTSESATPESGPGSPAGSPTSTEEGTSTEPSEGS |
| | APGTSESATPESGPGTSTEPSEGSAPGTSTEPSEGSAPGSPAGSPTSTEEGTSTEPSEGSAPGTSTEP |
| | SEGSAPGTSESATPESGPGTSESATPESGPGTSTEPSEGSAPGTSTEPSEGSAPGTSESATPESGPGT |
| | STEPSEGSAPGSEPATSGSETPGSPAGSPTSTEEGSSTPSGATGSPGTPGSGTASSSPGSSTPSGAT |
| | GSPGTSTEPSEGSAPGTSTEPSEGSAPGSEPATSGSETPGSPAGSPTSTEEGSPAGSPTSTEEGTSTE |
| | PSEGSAPGASASGAPSTGGTSESATPESGPGSPAGSPTSTEEGSPAGSPTSTEEGSTSSTAESPGPG |
| | STSESPSGTAPGTSPSGESSTAPGTPGSGTASSSPGSSTPSGATGSPGSSPSASTGTGPGSEPATSGS |
| | ETPGTSESATPESGPGSEPATSGSETPGSTSSTAESPGPGSTSSTAESPGPGTSPSGESSTAPGSEPA |
| | TSGSETPGSEPATSGSETPGTSTEPSEGSAPGSTSSTAESPGPGTSTPESGSASPGSTSESPSGTAPG |
| | TSTEPSEGSAPGTSTEPSEGSAPGTSTEPSEGSAPGSSTPSGATGSPGSSPSASTGTGPGASPGTSST |
| | GSPGSEPATSGSETPGTSESATPESGPGSPAGSPTSTEEGSSTPSGATGSPGSSPSASTGTGPGASP |
| | GTSSTGSPGTSESATPESGPGTSTEPSEGSAPGTSTEPSEGSAP |
| AM1318 | GTSTEPSEGSAPGSEPATSGSETPGSPAGSPTSTEEGSTSSTAESPGPGTSTPESGSASPGSTSESPS |
| | GTAPGSTSESPSGTAPGTSTPESGSASPGTSTPESGSASPGSEPATSGSETPGTSESATPESGPGSPA |
| | GSPTSTEEGTSTEPSEGSAPGTSESATPESGPGTSTEPSEGSAPGTSTEPSEGSAPGSPAGSPTSTEE |
| | GTSTEPSEGSAPGTSTEPSEGSAPGTSESATPESGPGTSESATPESGPGTSTEPSEGSAPGTSTEPSE |
| | GSAPGTSESATPESGPGTSTEPSEGSAPGSEPATSGSETPGSPAGSPTSTEEGSSTPSGATGSPGTP |
| | GSGTASSSPGSSTPSGATGSPGTSTEPSEGSAPGTSTEPSEGSAPGSEPATSGSETPGSPAGSPTSTE |
| | EGSPAGSPTSTEEGTSTEPSEGSAPGPEPTGPAPSGGSEPATSGSETPGTSESATPESGPGSPAGSP |
| | TSTEEGTSESATPESGPGSPAGSPTSTEEGSPAGSPTSTEEGTSESATPESGPGSPAGSPTSTEEGSP |
| | AGSPTSTEEGSTSSTAESPGPGSTSESPSGTAPGTSPSGESSTAPGSTSESPSGTAPGSTSESPSGTA |
| | PGTSPSGESSTAPGTSTEPSEGSAPGTSESATPESGPGTSESATPESGPGSEPATSGSETPGTSESAT |
| | PESGPGTSESATPESGPGTSTEPSEGSAPGTSESATPESGPGTSTEPSEGSAPGTSPSGESSTAPGTS |
| | PSGESSTAPGTSPSGESSTAPGTSTEPSEGSAPGSPAGSPTSTEEGTSTEPSEGSAPGSSPSASTGTG |
| | PGSSTPSGATGSPGSSTPSGATGSPGSSTPSGATGSPGSSTPSGATGSPGASPGTSSTGSPGASASG |
| | APSTGGTSPSGESSTAPGSTSSTAESPGPGTSPSGESSTAPGTSESATPESGPGTSTEPSEGSAPGTS |
| | TEPSEGSAPGSSPSASTGTGPGSSTPSGATGSPGASPGTSSTGSPGTSTPESGSASPGTSPSGESSTA |
| | PGTSPSGESSTAPGTSESATPESGPGSEPATSGSETPGTSTEPSEGSAPGSTSESPSGTAPGSTSESP |
| | SGTAPGTSTPESGSASPGSPAGSPTSTEEGTSESATPESGPGTSTEPSEGSAPGSPAGSPTSTEEGTS |
| | ESATPESGPGSEPATSGSETPGSSTPSGATGSPGASPGTSSTGSPGSSTPSGATGSPGSTSESPSGTA |
| | PGTSPSGESSTAPGSTSSTAESPGPGSSTPSGATGSPGASPGTSSTGSPGTPGSGTASSSPGSPAGSP |
| | TSTEEGSPAGSPTSTEEGTSTEPSEGSAP |
| BC 864 | GTSTEPSEPGSAGTSTEPSEPGSAGSEPATSGTEPSGSGASEPTSTEPGSEPATSGTEPSGSEPATSG |
| | TEPSGSEPATSGTEPSGSGASEPTSTEPGTSTEPSEPGSAGSEPATSGTEPSGTSTEPSEPGSAGSEP |
| | ATSGTEPSGSEPATSGTEPSGTSTEPSEPGSAGTSTEPSEPGSAGSEPATSGTEPSGSEPATSGTEPS |
| | GTSEPSTSEPGAGSGASEPTSTEPGTSEPSTSEPGAGSEPATSGTEPSGSEPATSGTEPSGTSTEPSE |
| | PGSAGTSTEPSEPGSAGSGASEPTSTEPGSEPATSGTEPSGSEPATSGTEPSGSEPATSGTEPSGSEP |
| | ATSGTEPSGTSTEPSEPGSAGSEPATSGTEPSGSGASEPTSTEPGTSTEPSEPGSAGSEPATSGTEPS |
| | GSGASEPTSTEPGTSTEPSEPGSAGSGASEPTSTEPGSEPATSGTEPSGSGASEPTSTEPGSEPATSG |
| | TEPSGSGASEPTSTEPGTSTEPSEPGSAGSEPATSGTEPSGSGASEPTSTEPGTSTEPSEPGSAGSEP |
| | ATSGTEPSGTSTEPSEPGSAGSEPATSGTEPSGTSTEPSEPGSAGTSTEPSEPGSAGTSTEPSEPGSA |
| | GTSTEPSEPGSAGTSTEPSEPGSAGTSTEPSEPGSAGTSEPSTSEPGAGSGASEPTSTEPGTSTEPSE |
| | PGSAGTSTEPSEPGSAGTSTEPSEPGSAGSEPATSGTEPSGSGASEPTSTEPGSEPATSGTEPSGSEP |
| | ATSGTEPSGSEPATSGTEPSGSEPATSGTEPSGTSEPSTSEPGAGSEPATSGTEPSGSGASEPTSTEP |
| | GTSTEPSEPGSAGSEPATSGTEPSGSGASEPTSTEPGTSTEPSEPGSA |
| BD864 | GSETATSGSETAGTSESATSESGAGSTAGSETSTEAGTSESATSESGAGSETATSGSETAGSETAT |
| | SGSETAGTSTEASEGSASGTSTEASEGSASGTSESATSESGAGSETATSGSETAGTSTEASEGSAS |
| | GSTAGSETSTEAGTSESATSESGAGTSESATSESGAGSETATSGSETAGTSESATSESGAGTSTEA |
| | SEGSASGSETATSGSETAGSETATSGSETAGTSTEASEGSASGSTAGSETSTEAGTSESATSESGA |
| | GTSTEASEGSASGSETATSGSETAGSTAGSETSTEAGSTAGSETSTEAGSETATSGSETAGTSESA |
| | TSESGAGTSESATSESGAGSETATSGSETAGTSESATSESGAGTSESATSESGAGSETATSGSETA |
| | GSETATSGSETAGTSTEASEGSASGSTAGSETSTEAGSETATSGSETAGTSESATSESGAGSTAGS |
| | ETSTEAGSTAGSETSTEAGSTAGSETSTEAGTSTEASEGSASGSTAGSETSTEAGSTAGSETSTEA |
| | GTSTEASEGSASGSTAGSETSTEAGSETATSGSETAGTSTEASEGSASGTSESATSESGAGSETAT |
| | SGSETAGTSESATSESGAGTSESATSESGAGSETATSGSETAGTSESATSESGAGSETATSGSETA |
| | GTSTEASEGSASGTSTEASEGSASGSTAGSETSTEAGSTAGSETSTEAGSETATSGSETAGTSESA |
| | TSESGAGTSESATSESGAGSETATSGSETAGSETATSGSETAGSETATSGSETAGTSTEASEGSAS |
| | GTSESATSESGAGSETATSGSETAGSETATSGSETAGTSESATSESGAGTSESATSESGAGSETAT |
| | SGSETA |
| AE948 | GTSTEPSEGSAPGTSESATPESGPGSEPATSGSETPGTSESATPESGPGTSTEPSEGSAPGSPAGSPT |
| | STEEGTSTEPSEGSAPGSEPATSGSETPGTSESATPESGPGTSESATPESGPGSEPATSGSETPGTST |
| | EPSEGSAPGTSESATPESGPGTSTEPSEGSAPGTSTEPSEGSAPGSEPATSGSETPGTSTEPSEGSAP |
| | GSEPATSGSETPGSEPATSGSETPGTSTEPSEGSAPGSEPATSGSETPGSEPATSGSETPGTSTEPSE |
| | GSAPGSEPATSGSETPGSPAGSPTSTEEGTSESATPESGPGTSTEPSEGSAPGSEPATSGSETPGTSE |
| | SATPESGPGSEPATSGSETPGSEPATSGSETPGTSESATPESGPGSPAGSPTSTEEGTSESATPESGP |
| | GSPAGSPTSTEEGTSTEPSEGSAPGTSESATPESGPGTSTEPSEGSAPGTSTEPSEGSAPGSPAGSPT |
| | STEEGTSTEPSEGSAPGTSESATPESGPGSPAGSPTSTEEGSPAGSPTSTEEGTSESATPESGPGTSE |
| | SATPESGPGTSTEPSEGSAPGSPAGSPTSTEEGTSESATPESGPGTSTEPSEGSAPGSEPATSGSETP |
| | GSPAGSPTSTEEGTSESATPESGPGTSTEPSEGSAPGSEPATSGSETPGSEPATSGSETPGSEPATSG |
| | SETPGSPAGSPTSTEEGTSESATPESGPGSEPATSGSETPGSEPATSGSETPGSPAGSPTSTEEGSPA |
| | GSPTSTEEGSPAGSPTSTEEGTSESATPESGPGSEPATSGSETPGTSTEPSEGSAPGTSTEPSEGSAP |
| | GSPAGSPTSTEEGSPAGSPTSTEEGSPAGSPTSTEEGTSTEPSEGSAPGTSESATPESGPGTSESATP |
| | ESGPGTSTEPSEGSAPGTSTEPSEGSAPGTSTEPSEGSAPGTSESATPESGPGTSESATPESGP |
| AE1044 | GSEPATSGSETPGSEPATSGSETPGTSTEPSEGSAPGTSESATPESGPGSPAGSPTSTEEGTSTEPSE |
| | GSAPGTSESATPESGPGTSESATPESGPGSEPATSGSETPGSEPATSGSETPGTSESATPESGPGTSE |
| | SATPESGPGSPAGSPTSTEEGTSTEPSEGSAPGSEPATSGSETPGTSTEPSEGSAPGSPAGSPTSTEE |
| | GSPAGSPTSTEEGSPAGSPTSTEEGTSTEPSEGSAPGTSESATPESGPGSEPATSGSETPGTSESATP |
| | ESGPGTSESATPESGPGSPAGSPTSTEEGTSTEPSEGSAPGSEPATSGSETPGTSTEPSEGSAPGTSE |
| | SATPESGPGSPAGSPTSTEEGTSTEPSEGSAPGTSTEPSEGSAPGSPAGSPTSTEEGTSTEPSEGSAP |
| | GTSESATPESGPGSPAGSPTSTEEGTSESATPESGPGSEPATSGSETPGTSTEPSEGSAPGSEPATSG |
| | SETPGTSESATPESGPGTSTEPSEGSAPGTSESATPESGPGTSTEPSEGSAPGTSTEPSEGSAPGSEP |
| | ATSGSETPGSPAGSPTSTEEGTSESATPESGPGTSTEPSEGSAPGSPAGSPTSTEEGTSTEPSEGSAP |
| | GTSESATPESGPGTSTEPSEGSAPGSEPATSGSETPGSEPATSGSETPGTSESATPESGPGTSESATP |
| | ESGPGTSESATPESGPGTSTEPSEGSAPGTSTEPSEGSAPGSPAGSPTSTEEGSPAGSPTSTEEGTSE |
| | SATPESGPGSPAGSPTSTEEGSPAGSPTSTEEGTSESATPESGPGSEPATSGSETPGTSTEPSEGSAP |
| | GSEPATSGSETPGSPAGSPTSTEEGTSESATPESGPGTSESATPESGPGSEPATSGSETPGTSTEPSE |
| | GSAPGTSTEPSEGSAPGSPAGSPTSTEEGTSTEPSEGSAPGTSTEPSEGSAPGSEPATSGSETPGSP |
| | AGSPTSTEEGTSESATPESGPGTSTEPSEGSAPGSPAGSPTSTEEGTSESATPESGPGTSESATPESG |
| | PGTST |
| AE1140 | GSEPATSGSETPGSEPATSGSETPGTSTEPSEGSAPGSEPATSGSETPGTSESATPESGPGSEPATSG |
| | SETPGSPAGSPTSTEEGTSESATPESGPGSEPATSGSETPGTSTEPSEGSAPGTSESATPESGPGTSE |
| | SATPESGPGTSTEPSEGSAPGTSTEPSEGSAPGSEPATSGSETPGSPAGSPTSTEEGTSTEPSEGSAP |
| | GSEPATSGSETPGSPAGSPTSTEEGTSTEPSEGSAPGTSESATPESGPGSPAGSPTSTEEGTSTEPSE |
| | GSAPGTSESATPESGPGSEPATSGSETPGTSESATPESGPGSEPATSGSETPGTSTEPSEGSAPGTST |
| | EPSEGSAPGTSTEPSEGSAPGSPAGSPTSTEEGSPAGSPTSTEEGTSESATPESGPGTSTEPSEGSAP |
| | GTSTEPSEGSAPGSPAGSPTSTEEGSPAGSPTSTEEGSPAGSPTSTEEGTSESATPESGPGSEPATSG |
| | SETPGTSESATPESGPGSPAGSPTSTEEGTSTEPSEGSAPGTSESATPESGPGSEPATSGSETPGSPA |
| | GSPTSTEEGTSTEPSEGSAPGTSESATPESGPGTSESATPESGPGSPAGSPTSTEEGTSTEPSEGSAP |
| | GSPAGSPTSTEEGSPAGSPTSTEEGTSESATPESGPGSEPATSGSETPGTSTEPSEGSAPGSEPATSG |
| | SETPGTSESATPESGPGTSTEPSEGSAPGTSESATPESGPGTSESATPESGPGTSTEPSEGSAPGTST |
| | EPSEGSAPGSEPATSGSETPGTSESATPESGPGTSTEPSEGSAPGSEPATSGSETPGSPAGSPTSTEE |
| | GTSESATPESGPGSPAGSPTSTEEGTSTEPSEGSAPGSEPATSGSETPGSEPATSGSETPGSEPATSG |
| | SETPGTSESATPESGPGTSESATPESGPGTSTEPSEGSAPGTSTEPSEGSAPGSEPATSGSETPGTST |
| | EPSEGSAPGTSESATPESGPGSPAGSPTSTEEGSPAGSPTSTEEGTSTEPSEGSAPGSPAGSPTSTEE |
| | GSPA |
| AE1236 | GSPAGSPTSTEEGTSTEPSEGSAPGSPAGSPTSTEEGTSESATPESGPGSEPATSGSETPGTSTEPSE |
| | GSAPGTSTEPSEGSAPGTSESATPESGPGTSTEPSEGSAPGTSTEPSEGSAPGSEPATSGSETPGSP |
| | AGSPTSTEEGTSESATPESGPGSPAGSPTSTEEGTSTEPSEGSAPGSPAGSPTSTEEGTSTEPSEGSA |
| | PGSPAGSPTSTEEGTSTEPSEGSAPGSPAGSPTSTEEGSPAGSPTSTEEGTSTEPSEGSAPGSEPATS |
| | GSETPGSPAGSPTSTEEGTSESATPESGPGSEPATSGSETPGSEPATSGSETPGTSESATPESGPGSP |
| | AGSPTSTEEGTSESATPESGPGSEPATSGSETPGSEPATSGSETPGTSESATPESGPGTSESATPESG |
| | PGTSTEPSEGSAPGTSTEPSEGSAPGTSESATPESGPGSPAGSPTSTEEGTSTEPSEGSAPGSEPATS |
| | GSETPGSPAGSPTSTEEGTSTEPSEGSAPGTSTEPSEGSAPGSPAGSPTSTEEGSPAGSPTSTEEGSP |
| | AGSPTSTEEGTSESATPESGPGSEPATSGSETPGTSESATPESGPGTSESATPESGPGTSTEPSEGSA |
| | PGTSESATPESGPGSPAGSPTSTEEGTSESATPESGPGTSESATPESGPGTSTEPSEGSAPGSEPATS |
| | GSETPGTSTEPSEGSAPGSPAGSPTSTEEGTSESATPESGPGTSTEPSEGSAPGTSTEPSEGSAPGSE |
| | PATSGSETPGSEPATSGSETPGTSESATPESGPGSEPATSGSETPGSPAGSPTSTEEGTSESATPESG |
| | PGSEPATSGSETPGTSTEPSEGSAPGTSESATPESGPGTSESATPESGPGTSTEPSEGSAPGSEPATS |
| | GSETPGTSTEPSEGSAPGSPAGSPTSTEEGSPAGSPTSTEEGTSESATPESGPGSEPATSGSETPGSP |
| | AGSPTSTEEGSPAGSPTSTEEGTSESATPESGPGSEPATSGSETPGTSTEPSEGSAPGTSTEPSEGSA |
| | PGSEP |
| AE1332 | GSPAGSPTSTEEGTSTEPSEGSAPGTSESATPESGPGTSESATPESGPGSEPATSGSETPGTSTEPSE |
| | GSAPGTSESATPESGPGSPAGSPTSTEEGTSTEPSEGSAPGTSESATPESGPGSPAGSPTSTEEGTST |
| | EPSEGSAPGTSESATPESGPGTSTEPSEGSAPGSEPATSGSETPGSEPATSGSETPGSEPATSGSETP |
| | GTSESATPESGPGSPAGSPTSTEEGTSESATPESGPGSEPATSGSETPGTSESATPESGPGTSESATP |
| | ESGPGSPAGSPTSTEEGTSTEPSEGSAPGTSESATPESGPGTSTEPSEGSAPGTSTEPSEGSAPGTST |
| | EPSEGSAPGSEPATSGSETPGTSTEPSEGSAPGTSESATPESGPGTSESATPESGPGTSTEPSEGSAP |
| | GTSESATPESGPGTSESATPESGPGSEPATSGSETPGTSTEPSEGSAPGTSESATPESGPGSPAGSPT |
| | STEEGSPAGSPTSTEEGTSTEPSEGSAPGSEPATSGSETPGSEPATSGSETPGTSTEPSEGSAPGSEP |
| | ATSGSETPGSPAGSPTSTEEGTSESATPESGPGSEPATSGSETPGSEPATSGSETPGTSESATPESGP |
| | GTSESATPESGPGTSTEPSEGSAPGSPAGSPTSTEEGTSESATPESGPGTSTEPSEGSAPGSEPATSG |
| | SETPGSPAGSPTSTEEGTSTEPSEGSAPGTSTEPSEGSAPGSEPATSGSETPGTSESATPESGPGTST |
| | EPSEGSAPGSPAGSPTSTEEGTSESATPESGPGTSESATPESGPGTSTEPSEGSAPGSEPATSGSETP |
| | GSEPATSGSETPGTSTEPSEGSAPGTSESATPESGPGSPAGSPTSTEEGTSTEPSEGSAPGTSTEPSE |
| | GSAPGSPAGSPTSTEEGSPAGSPTSTEEGTSTEPSEGSAPGSEPATSGSETPGSPAGSPTSTEEGTSE |
| | SATPESGPGSEPATSGSETPGSEPATSGSETPGTSESATPESGPGTSESATPESGPGTSTEPSEGSAP |
| | GTST |
| AE1428 | GSEPATSGSETPGSPAGSPTSTEEGTSTEPSEGSAPGSEPATSGSETPGTSESATPESGPGTSTEPSE |
| | GSAPGTSESATPESGPGSPAGSPTSTEEGTSTEPSEGSAPGTSTEPSEGSAPGSPAGSPTSTEEGTST |
| | EPSEGSAPGTSESATPESGPGSEPATSGSETPGTSESATPESGPGSEPATSGSETPGTSTEPSEGSAP |
| | GTSTEPSEGSAPGSEPATSGSETPGTSTEPSEGSAPGTSTEPSEGSAPGSPAGSPTSTEEGTSTEPSE |
| | GSAPGTSESATPESGPGTSESATPESGPGTSTEPSEGSAPGTSTEPSEGSAPGSPAGSPTSTEEGSP |
| | AGSPTSTEEGTSTEPSEGSAPGSEPATSGSETPGSPAGSPTSTEEGTSTEPSEGSAPGSPAGSPTSTE |
| | EGSPAGSPTSTEEGTSESATPESGPGSEPATSGSETPGSEPATSGSETPGTSTEPSEGSAPGSPAGSP |
| | TSTEEGSPAGSPTSTEEGTSESATPESGPGSEPATSGSETPGSEPATSGSETPGTSESATPESGPGSE |
| | PATSGSETPGSEPATSGSETPGTSESATPESGPGTSESATPESGPGTSESATPESGPGTSESATPESG |
| | PGSPAGSPTSTEEGTSTEPSEGSAPGTSESATPESGPGTSTEPSEGSAPGTSTEPSEGSAPGTSESAT |
| | PESGPGTSTEPSEGSAPGTSESATPESGPGSPAGSPTSTEEGTSTEPSEGSAPGSEPATSGSETPGTS |
| | TEPSEGSAPGSEPATSGSETPGTSTEPSEGSAPGTSTEPSEGSAPGSPAGSPTSTEEGTSESATPESG |
| | PGTSTEPSEGSAPGSEPATSGSETPGTSESATPESGPGTSESATPESGPGTSESATPESGPGTSTEPS |
| | EGSAPGSEPATSGSETPGSPAGSPTSTEEGTSESATPESGPGTSTEPSEGSAPGTSTEPSEGSAPGSP |
| | AGSPTSTEEGTSESATPESGPGTSTEPSEGSAPGTSTEPSEGSAPGSPAGSPTSTEEGTSESATPESG |
| | PGSPA |
| AE1524 | GTSTEPSEGSAPGTSTEPSEGSAPGTSTEPSEGSAPGTSTEPSEGSAPGSPAGSPTSTEEGSPAGSPT |
| | STEEGTSESATPESGPGSEPATSGSETPGTSTEPSEGSAPGTSESATPESGPGTSTEPSEGSAPGTSE |
| | SATPESGPGTSESATPESGPGSPAGSPTSTEEGTSTEPSEGSAPGSEPATSGSETPGTSTEPSEGSAP |
| | GSPAGSPTSTEEGTSTEPSEGSAPGSPAGSPTSTEEGSPAGSPTSTEEGSPAGSPTSTEEGTSESATP |
| | ESGPGSEPATSGSETPGTSESATPESGPGTSESATPESGPGSPAGSPTSTEEGTSTEPSEGSAPGSEP |
| | ATSGSETPGTSTEPSEGSAPGSPAGSPTSTEEGSPAGSPTSTEEGTSTEPSEGSAPGSEPATSGSETP |
| | GSPAGSPTSTEEGTSESATPESGPGSEPATSGSETPGSPAGSPTSTEEGTSESATPESGPGTSESATP |
| | ESGPGTSTEPSEGSAPGSEPATSGSETPGSEPATSGSETPGTSESATPESGPGSEPATSGSETPGTST |
| | EPSEGSAPGSPAGSPTSTEEGTSTEPSEGSAPGSEPATSGSETPGSEPATSGSETPGSPAGSPTSTEE |
| | GTSESATPESGPGTSTEPSEGSAPGSPAGSPTSTEEGSPAGSPTSTEEGTSESATPESGPGSPAGSPT |
| | STEEGTSTEPSEGSAPGSEPATSGSETPGSEPATSGSETPGSEPATSGSETPGTSESATPESGPGTSE |
| | SATPESGPGTSTEPSEGSAPGTSTEPSEGSAPGSEPATSGSETPGTSTEPSEGSAPGTSESATPESGP |
| | GSPAGSPTSTEEGSPAGSPTSTEEGTSTEPSEGSAPGTSESATPESGPGSPAGSPTSTEEGTSTEPSE |
| | GSAPGSEPATSGSETPGTSESATPESGPGSEPATSGSETPGTSESATPESGPGSEPATSGSETPGSEP |
| | ATSGSETPGTSTEPSEGSAPGTSESATPESGPGTSTEPSEGSAPGTSTEPSEGSAPGTSESATPESGP |
| | GSPA |
| AE1620 | GSEPATSGSETPGTSTEPSEGSAPGSEPATSGSETPGTSTEPSEGSAPGTSESATPESGPGTSESATP |
| | ESGPGSEPATSGSETPGTSTEPSEGSAPGTSTEPSEGSAPGTSESATPESGPGTSTEPSEGSAPGTST |
| | EPSEGSAPGSEPATSGSETPGSPAGSPTSTEEGTSESATPESGPGTSESATPESGPGTSTEPSEGSAP |
| | GTSESATPESGPGSEPATSGSETPGTSESATPESGPGTSESATPESGPGTSTEPSEGSAPGTSTEPSE |
| | GSAPGSEPATSGSETPGSPAGSPTSTEEGTSESATPESGPGSEPATSGSETPGSEPATSGSETPGTSE |
| | SATPESGPGTSESATPESGPGSPAGSPTSTEEGTSTEPSEGSAPGTSESATPESGPGTSTEPSEGSAP |
| | GTSTEPSEGSAPGTSESATPESGPGTSTEPSEGSAPGSPAGSPTSTEEGSPAGSPTSTEEGTSESATP |
| | ESGPGSEPATSGSETPGSEPATSGSETPGTSTEPSEGSAPGTSESATPESGPGSPAGSPTSTEEGSPA |
| | GSPTSTEEGTSTEPSEGSAPGSPAGSPTSTEEGSPAGSPTSTEEGTSESATPESGPGSEPATSGSETP |
| | GTSTEPSEGSAPGTSTEPSEGSAPGSEPATSGSETPGSPAGSPTSTEEGTSESATPESGPGSPAGSPT |
| | STEEGTSTEPSEGSAPGTSESATPESGPGSEPATSGSETPGTSESATPESGPGTSESATPESGPGSPA |
| | GSPTSTEEGTSTEPSEGSAPGSEPATSGSETPGSPAGSPTSTEEGTSTEPSEGSAPGSEPATSGSETP |
| | GSEPATSGSETPGTSESATPESGPGTSESATPESGPGTSTEPSEGSAPGTSESATPESGPGSEPATSG |
| | SETPGTSESATPESGPGTSESATPESGPGTSTEPSEGSAPGTSESATPESGPGSEPATSGSETPGTST |
| | EPSEGSAPGSPAGSPTSTEEGTSESATPESGPGTSTEPSEGSAPGTSTEPSEGSAPGSPAGSPTSTEE |
| | GTST |
| AE1716 | GTSESATPESGPGTSTEPSEGSAPGTSESATPESGPGTSTEPSEGSAPGSEPATSGSETPGSPAGSPT |
| | STEEGTSESATPESGPGSEPATSGSETPGSEPATSGSETPGTSESATPESGPGTSESATPESGPGTST |
| | EPSEGSAPGSEPATSGSETPGSEPATSGSETPGTSESATPESGPGTSESATPESGPGSEPATSGSETP |
| | GTSESATPESGPGSPAGSPTSTEEGSPAGSPTSTEEGTSTEPSEGSAPGSEPATSGSETPGSPAGSPT |
| | STEEGTSESATPESGPGSPAGSPTSTEEGTSTEPSEGSAPGSEPATSGSETPGSPAGSPTSTEEGTSE |
| | SATPESGPGSPAGSPTSTEEGSPAGSPTSTEEGTSTEPSEGSAPGSEPATSGSETPGSPAGSPTSTEE |
| | GTSESATPESGPGTSTEPSEGSAPGSPAGSPTSTEEGTSESATPESGPGSEPATSGSETPGTSTEPSE |
| | GSAPGSPAGSPTSTEEGTSTEPSEGSAPGTSESATPESGPGTSESATPESGPGTSTEPSEGSAPGTST |
| | EPSEGSAPGSEPATSGSETPGTSESATPESGPGTSESATPESGPGTSTEPSEGSAPGTSTEPSEGSAP |
| | GSPAGSPTSTEEGSPAGSPTSTEEGTSTEPSEGSAPGSEPATSGSETPGSEPATSGSETPGTSESATP |
| | ESGPGSPAGSPTSTEEGTSESATPESGPGTSTEPSEGSAPGTSTEPSEGSAPGSPAGSPTSTEEGSPA |
| | GSPTSTEEGTSESATPESGPGSPAGSPTSTEEGTSTEPSEGSAPGSEPATSGSETPGSPAGSPTSTEE |
| | GSPAGSPTSTEEGTSTEPSEGSAPGTSESATPESGPGTSESATPESGPGSPAGSPTSTEEGTSTEPSE |
| | GSAPGSPAGSPTSTEEGTSTEPSEGSAPGSPAGSPTSTEEGTSTEPSEGSAPGTSTEPSEGSAPGSEP |
| | ATSGSETPGSPAGSPTSTEEGTSESATPESGPGSEPATSGSETPGSPAGSPTSTEEGTSESATPESGP |
| | GTSE |
| AE1812 | GTSESATPESGPGSPAGSPTSTEEGSPAGSPTSTEEGTSESATPESGPGSEPATSGSETPGTSTEPSE |
| | GSAPGTSTEPSEGSAPGTSTEPSEGSAPGSPAGSPTSTEEGTSTEPSEGSAPGTSESATPESGPGTSE |
| | SATPESGPGSPAGSPTSTEEGSPAGSPTSTEEGSPAGSPTSTEEGTSESATPESGPGSEPATSGSETP |
| | GTSESATPESGPGTSTEPSEGSAPGTSESATPESGPGTSTEPSEGSAPGTSTEPSEGSAPGTSESATP |
| | ESGPGSPAGSPTSTEEGTSESATPESGPGTSTEPSEGSAPGSPAGSPTSTEEGTSESATPESGPGSEP |
| | ATSGSETPGTSTEPSEGSAPGSEPATSGSETPGSEPATSGSETPGTSESATPESGPGTSESATPESGP |
| | GTSTEPSEGSAPGTSESATPESGPGSPAGSPTSTEEGTSTEPSEGSAPGSEPATSGSETPGTSESATP |
| | ESGPGSEPATSGSETPGTSTEPSEGSAPGTSESATPESGPGSPAGSPTSTEEGSPAGSPTSTEEGSPA |
| | GSPTSTEEGTSESATPESGPGSEPATSGSETPGTSESATPESGPGTSESATPESGPGSEPATSGSETP |
| | GTSTEPSEGSAPGTSTEPSEGSAPGTSTEPSEGSAPGTSESATPESGPGSEPATSGSETPGTSESATP |
| | ESGPGSEPATSGSETPGTSTEPSEGSAPGSEPATSGSETPGSPAGSPTSTEEGTSESATPESGPGTSE |
| | SATPESGPGTSESATPESGPGSPAGSPTSTEEGTSTEPSEGSAPGTSESATPESGPGTSESATPESGP |
| | GTSTEPSEGSAPGTSTEPSEGSAPGSPAGSPTSTEEGTSESATPESGPGSEPATSGSETPGTSESATP |
| | ESGPGTSESATPESGPGTSTEPSEGSAPGSEPATSGSETPGTSESATPESGPGSPAGSPTSTEEGTST |
| | EPSEGSAPGSEPATSGSETPGSPAGSPTSTEEGTSESATPESGPGSPAGSPTSTEEGTSTEPSEGSAP |
| | GSEP |
| AE1908 | GSEPATSGSETPGTSTEPSEGSAPGTSESATPESGPGSEPATSGSETPGTSTEPSEGSAPGSPAGSPT |
| | STEEGTSESATPESGPGSPAGSPTSTEEGTSTEPSEGSAPGSEPATSGSETPGTSTEPSEGSAPGTSE |
| | SATPESGPGSEPATSGSETPGSEPATSGSETPGTSESATPESGPGTSTEPSEGSAPGTSESATPESGP |
| | GTSESATPESGPGSPAGSPTSTEEGSPAGSPTSTEEGTSESATPESGPGTSESATPESGPGTSTEPSE |
| | GSAPGTSTEPSEGSAPGSPAGSPTSTEEGTSTEPSEGSAPGSEPATSGSETPGSEPATSGSETPGSEP |
| | ATSGSETPGSEPATSGSETPGTSESATPESGPGTSESATPESGPGTSESATPESGPGTSTEPSEGSAP |
| | GTSESATPESGPGTSESATPESGPGTSTEPSEGSAPGSPAGSPTSTEEGTSTEPSEGSAPGSEPATSG |
| | SETPGSPAGSPTSTEEGTSESATPESGPGTSTEPSEGSAPGSEPATSGSETPGTSESATPESGPGTSE |
| | SATPESGPGTSTEPSEGSAPGSEPATSGSETPGSPAGSPTSTEEGTSESATPESGPGTSESATPESGP |
| | GTSTEPSEGSAPGSEPATSGSETPGSPAGSPTSTEEGTSESATPESGPGSEPATSGSETPGTSTEPSE |
| | GSAPGSEPATSGSETPGSPAGSPTSTEEGTSESATPESGPGTSTEPSEGSAPGSEPATSGSETPGTSE |
| | SATPESGPGTSESATPESGPGSPAGSPTSTEEGTSTEPSEGSAPGSPAGSPTSTEEGSPAGSPTSTEE |
| | GSPAGSPTSTEEGTSESATPESGPGSEPATSGSETPGTSTEPSEGSAPGTSTEPSEGSAPGTSESATP |
| | ESGPGTSTEPSEGSAPGTSESATPESGPGSEPATSGSETPGTSTEPSEGSAPGTSESATPESGPGSPA |
| | GSPTSTEEGSPAGSPTSTEEGSPAGSPTSTEEGTSESATPESGPGSPAGSPTSTEEGTSESATPESGP |
| | GSEP |
| AE2004A | GTSTEPSEGSAPGSEPATSGSETPGTSTEPSEGSAPGTSTEPSEGSAPGSEPATSGSETPGSPAGSPT |
| | STEEGSPAGSPTSTEEGTSESATPESGPGSEPATSGSETPGTSTEPSEGSAPGTSTEPSEGSAPGSPA |
| | GSPTSTEEGTSESATPESGPGTSESATPESGPGSPAGSPTSTEEGTSTEPSEGSAPGTSESATPESGP |
| | GTSTEPSEGSAPGSEPATSGSETPGTSESATPESGPGSEPATSGSETPGTSTEPSEGSAPGTSTEPSE |
| | GSAPGSPAGSPTSTEEGSPAGSPTSTEEGTSESATPESGPGSPAGSPTSTEEGTSTEPSEGSAPGSEP |
| | ATSGSETPGSEPATSGSETPGSPAGSPTSTEEGTSESATPESGPGTSTEPSEGSAPGSEPATSGSETP |
| | GTSESATPESGPGSEPATSGSETPGSPAGSPTSTEEGTSESATPESGPGTSESATPESGPGSEPATSG |
| | SETPGTSESATPESGPGTSESATPESGPGTSESATPESGPGTSTEPSEGSAPGTSESATPESGPGSEP |
| | ATSGSETPGTSTEPSEGSAPGSPAGSPTSTEEGTSTEPSEGSAPGSEPATSGSETPGSPAGSPTSTEE |
| | GTSESATPESGPGTSESATPESGPGTSTEPSEGSAPGTSESATPESGPGTSTEPSEGSAPGTSESATP |
| | ESGPGSEPATSGSETPGTSTEPSEGSAPGTSTEPSEGSAPGSPAGSPTSTEEGSPAGSPTSTEEGTSE |
| | SATPESGPGTSTEPSEGSAPGTSESATPESGPGSPAGSPTSTEEGSPAGSPTSTEEGSPAGSPTSTEE |
| | GTSTEPSEGSAPGSPAGSPTSTEEGSPAGSPTSTEEGTSESATPESGPGSEPATSGSETPGSEPATSG |
| | SETPGTSESATPESGPGSEPATSGSETPGTSESATPESGPGTSTEPSEGSAPGTSTEPSEGSAPGSPA |
| | GSPTSTEEGSPAGSPTSTEEGTSESATPESGPGSPAGSPTSTEEGTSTEPSEGSAPGTSESATPESGP |
| | GTSE |
| AG948 | GSSTPSGATGSPGTPGSGTASSSPGASPGTSSTGSPGSSPSASTGTGPGTPGSGTASSSPGTPGSGT |
| | ASSSPGTPGSGTASSSPGSSPSASTGTGPGTPGSGTASSSPGSSPSASTGTGPGSSTPSGATGSPGSS |
| | TPSGATGSPGSSPSASTGTGPGSSTPSGATGSPGASPGTSSTGSPGASPGTSSTGSPGSSPSASTGT |
| | GPGSSPSASTGTGPGASPGTSSTGSPGSSPSASTGTGPGASPGTSSTGSPGSSPSASTGTGPGASPG |
| | TSSTGSPGTPGSGTASSSPGTPGSGTASSSPGSSTPSGATGSPGSSTPSGATGSPGSSTPSGATGSP |
| | GASPGTSSTGSPGSSPSASTGTGPGASPGTSSTGSPGASPGTSSTGSPGSSTPSGATGSPGSSPSAS |
| | TGTGPGSSPSASTGTGPGTPGSGTASSSPGASPGTSSTGSPGTPGSGTASSSPGSSTPSGATGSPGS |
| | STPSGATGSPGSSPSASTGTGPGSSPSASTGTGPGSSTPSGATGSPGASPGTSSTGSPGTPGSGTAS |
| | SSPGSSPSASTGTGPGSSTPSGATGSPGASPGTSSTGSPGSSTPSGATGSPGTPGSGTASSSPGSSPS |
| | ASTGTGPGSSTPSGATGSPGASPGTSSTGSPGASPGTSSTGSPGTPGSGTASSSPGTPGSGTASSSP |
| | GSSPSASTGTGPGASPGTSSTGSPGASPGTSSTGSPGTPGSGTASSSPGTPGSGTASSSPGSSTPSG |
| | ATGSPGSSPSASTGTGPGSSTPSGATGSPGSSPSASTGTGPGSSPSASTGTGPGTPGSGTASSSPGS |
| | STPSGATGSPGASPGTSSTGSPGTPGSGTASSSPGTPGSGTASSSPGSSTPSGATGSPGTPGSGTAS |
| | SSPGASPGTSSTGSPGSSTPSGATGSPGSSTPSGATGSPGTPGSGTASSSPGSSTPSGATGSPGSSTP |
| | SGATGSP |
| AG1044 | GTPGSGTASSSPGTPGSGTASSSPGSSPSASTGTGPGTPGSGTASSSPGASPGTSSTGSPGTPGSGT |
| | ASSSPGSSPSASTGTGPGSSTPSGATGSPGASPGTSSTGSPGASPGTSSTGSPGSSPSASTGTGPGSS |
| | TPSGATGSPGSSPSASTGTGPGSSPSASTGTGPGTPGSGTASSSPGTPGSGTASSSPGASPGTSSTG |
| | SPGSSTPSGATGSPGSSPSASTGTGPGTPGSGTASSSPGASPGTSSTGSPGTPGSGTASSSPGSSPSA |
| | STGTGPGSSPSASTGTGPGASPGTSSTGSPGASPGTSSTGSPGSSTPSGATGSPGSSTPSGATGSPG |
| | TPGSGTASSSPGSSPSASTGTGPGSSTPSGATGSPGASPGTSSTGSPGSSTPSGATGSPGSSTPSGA |
| | TGSPGSSPSASTGTGPGASPGTSSTGSPGTPGSGTASSSPGASPGTSSTGSPGSSPSASTGTGPGTP |
| | GSGTASSSPGASPGTSSTGSPGASPGTSSTGSPGSSTPSGATGSPGTPGSGTASSSPGSSTPSGATG |
| | SPGASPGTSSTGSPGTPGSGTASSSPGTPGSGTASSSPGSSTPSGATGSPGSSTPSGATGSPGTPGS |
| | GTASSSPGSSTPSGATGSPGSSTPSGATGSPGASPGTSSTGSPGSSPSASTGTGPGSSPSASTGTGP |
| | GASPGTSSTGSPGASPGTSSTGSPGTPGSGTASSSPGSSPSASTGTGPGTPGSGTASSSPGSSTPSG |
| | ATGSPGSSTPSGATGSPGASPGTSSTGSPGSSPSASTGTGPGASPGTSSTGSPGSSPSASTGTGPGS |
| | SPSASTGTGPGASPGTSSTGSPGASPGTSSTGSPGSSTPSGATGSPGASPGTSSTGSPGASPGTSST |
| | GSPGTPGSGTASSSPGTPGSGTASSSPGTPGSGTASSSPGSSTPSGATGSPGSSTPSGATGSPGSSPS |
| | ASTGTGPGSSPSASTGTGPGTPGSGTASSSPGSSPSASTGTGPGASPGTSSTGSPGSSTPSGATGSP |
| | GTPGSGTASSSPGSST |
| AG1140 | GASPGTSSTGSPGSSPSASTGTGPGSSTPSGATGSPGASPGTSSTGSPGASPGTSSTGSPGSSTPSG |
| | ATGSPGTPGSGTASSSPGASPGTSSTGSPGTPGSGTASSSPGTPGSGTASSSPGSSTPSGATGSPGS |
| | STPSGATGSPGTPGSGTASSSPGSSTPSGATGSPGSSTPSGATGSPGSSPSASTGTGPGSSPSASTG |
| | TGPGASPGTSSTGSPGTPGSGTASSSPGASPGTSSTGSPGSSTPSGATGSPGTPGSGTASSSPGSSP |
| | SASTGTGPGSSTPSGATGSPGSSPSASTGTGPGSSTPSGATGSPGASPGTSSTGSPGSSPSASTGTG |
| | PGTPGSGTASSSPGASPGTSSTGSPGSSTPSGATGSPGASPGTSSTGSPGSSPSASTGTGPGTPGSG |
| | TASSSPGTPGSGTASSSPGASPGTSSTGSPGTPGSGTASSSPGTPGSGTASSSPGSSPSASTGTGPG |
| | ASPGTSSTGSPGSSTPSGATGSPGASPGTSSTGSPGSSPSASTGTGPGTPGSGTASSSPGSSPSAST |
| | GTGPGSSPSASTGTGPGASPGTSSTGSPGASPGTSSTGSPGTPGSGTASSSPGASPGTSSTGSPGAS |
| | PGTSSTGSPGTPGSGTASSSPGTPGSGTASSSPGSSTPSGATGSPGSSTPSGATGSPGTPGSGTASS |
| | SPGSSPSASTGTGPGTPGSGTASSSPGSSTPSGATGSPGSSTPSGATGSPGASPGTSSTGSPGTPGS |
| | GTASSSPGASPGTSSTGSPGSSTPSGATGSPGSSTPSGATGSPGTPGSGTASSSPGSSTPSGATGSP |
| | GSSTPSGATGSPGASPGTSSTGSPGSSPSASTGTGPGSSTPSGATGSPGSSTPSGATGSPGSSPSAS |
| | TGTGPGSSPSASTGTGPGSSTPSGATGSPGASPGTSSTGSPGSSPSASTGTGPGTPGSGTASSSPGA |
| | SPGTSSTGSPGSSPSASTGTGPGASPGTSSTGSPGSSTPSGATGSPGSSPSASTGTGPGSSPSASTGT |
| | GPGASPGTSSTGSPGSST |
| AG1236 | GSSPSASTGTGPGTPGSGTASSSPGSSPSASTGTGPGSSPSASTGTGPGTPGSGTASSSPGASPGTS |
| | STGSPGSSTPSGATGSPGTPGSGTASSSPGASPGTSSTGSPGTPGSGTASSSPGTPGSGTASSSPGS |
| | SPSASTGTGPGSSPSASTGTGPGSSTPSGATGSPGASPGTSSTGSPGSSPSASTGTGPGTPGSGTAS |
| | SSPGTPGSGTASSSPGSSTPSGATGSPGASPGTSSTGSPGSSPSASTGTGPGTPGSGTASSSPGTPG |
| | SGTASSSPGASPGTSSTGSPGSSTPSGATGSPGSSTPSGATGSPGSSPSASTGTGPGSSPSASTGTG |
| | PGASPGTSSTGSPGSSPSASTGTGPGSSTPSGATGSPGASPGTSSTGSPGASPGTSSTGSPGSSTPS |
| | GATGSPGSSPSASTGTGPGASPGTSSTGSPGASPGTSSTGSPGTPGSGTASSSPGSSTPSGATGSPG |
| | ASPGTSSTGSPGSSTPSGATGSPGTPGSGTASSSPGSSPSASTGTGPGSSTPSGATGSPGTPGSGTA |
| | SSSPGSSTPSGATGSPGSSTPSGATGSPGASPGTSSTGSPGSSPSASTGTGPGSSPSASTGTGPGASP |
| | GTSSTGSPGSSPSASTGTGPGTPGSGTASSSPGTPGSGTASSSPGASPGTSSTGSPGTPGSGTASSS |
| | PGASPGTSSTGSPGTPGSGTASSSPGASPGTSSTGSPGSSTPSGATGSPGASPGTSSTGSPGSSPSAS |
| | TGTGPGTPGSGTASSSPGTPGSGTASSSPGSSPSASTGTGPGTPGSGTASSSPGASPGTSSTGSPGS |
| | STPSGATGSPGTPGSGTASSSPGASPGTSSTGSPGTPGSGTASSSPGTPGSGTASSSPGSSTPSGAT |
| | GSPGTPGSGTASSSPGSSPSASTGTGPGSSTPSGATGSPGTPGSGTASSSPGSSTPSGATGSPGSST |
| | PSGATGSPGSSPSASTGTGPGASPGTSSTGSPGTPGSGTASSSPGSSPSASTGTGPGSSPSASTGTG |
| | PGASPGTSSTGSPGASP |
| AG1332 | GSSTPSGATGSPGSSPSASTGTGPGTPGSGTASSSPGSSPSASTGTGPGASPGTSSTGSPGSSPSAS |
| | TGTGPGTPGSGTASSSPGASPGTSSTGSPGSSTPSGATGSPGSSPSASTGTGPGSSTPSGATGSPGA |
| | SPGTSSTGSPGTPGSGTASSSPGASPGTSSTGSPGSSTPSGATGSPGSSTPSGATGSPGSSPSASTGT |
| | GPGTPGSGTASSSPGSSTPSGATGSPGSSTPSGATGSPGSSPSASTGTGPGTPGSGTASSSPGASPG |
| | TSSTGSPGTPGSGTASSSPGASPGTSSTGSPGASPGTSSTGSPGTPGSGTASSSPGTPGSGTASSSP |
| | GSSTPSGATGSPGSSTPSGATGSPGSSTPSGATGSPGASPGTSSTGSPGSSPSASTGTGPGSSTPSG |
| | ATGSPGSSPSASTGTGPGSSTPSGATGSPGTPGSGTASSSPGSSPSASTGTGPGSSTPSGATGSPGA |
| | SPGTSSTGSPGASPGTSSTGSPGSSPSASTGTGPGASPGTSSTGSPGSSTPSGATGSPGASPGTSST |
| | GSPGTPGSGTASSSPGTPGSGTASSSPGSSPSASTGTGPGTPGSGTASSSPGSSTPSGATGSPGSSPS |
| | ASTGTGPGSSTPSGATGSPGTPGSGTASSSPGTPGSGTASSSPGSSPSASTGTGPGSSTPSGATGSP |
| | GSSTPSGATGSPGSSPSASTGTGPGSSPSASTGTGPGASPGTSSTGSPGASPGTSSTGSPGSSTPSG |
| | ATGSPGASPGTSSTGSPGSSPSASTGTGPGTPGSGTASSSPGASPGTSSTGSPGASPGTSSTGSPGT |
| | PGSGTASSSPGTPGSGTASSSPGSSPSASTGTGPGTPGSGTASSSPGASPGTSSTGSPGSSTPSGAT |
| | GSPGTPGSGTASSSPGTPGSGTASSSPGSSTPSGATGSPGSSTPSGATGSPGTPGSGTASSSPGSSPS |
| | ASTGTGPGSSPSASTGTGPGASPGTSSTGSPGSSPSASTGTGPGSSPSASTGTGPGASPGTSSTGSP |
| | GASPGTSSTGSPGTPG |
| AG1428 | GTPGSGTASSSPGSSTPSGATGSPGASPGTSSTGSPGSSTPSGATGSPGTPGSGTASSSPGTPGSGT |
| | ASSSPGSSTPSGATGSPGSSTPSGATGSPGASPGTSSTGSPGSSPSASTGTGPGSSTPSGATGSPGA |
| | SPGTSSTGSPGSSPSASTGTGPGTPGSGTASSSPGASPGTSSTGSPGASPGTSSTGSPGTPGSGTAS |
| | SSPGTPGSGTASSSPGASPGTSSTGSPGASPGTSSTGSPGTPGSGTASSSPGTPGSGTASSSPGSSPS |
| | ASTGTGPGSSPSASTGTGPGASPGTSSTGSPGSSPSASTGTGPGSSPSASTGTGPGASPGTSSTGSP |
| | GASPGTSSTGSPGTPGSGTASSSPGTPGSGTASSSPGSSPSASTGTGPGASPGTSSTGSPGSSTPSG |
| | ATGSPGASPGTSSTGSPGSSPSASTGTGPGSSTPSGATGSPGSSPSASTGTGPGSSPSASTGTGPGA |
| | SPGTSSTGSPGSSPSASTGTGPGSSPSASTGTGPGASPGTSSTGSPGASPGTSSTGSPGSSPSASTGT |
| | GPGTPGSGTASSSPGASPGTSSTGSPGSSTPSGATGSPGTPGSGTASSSPGTPGSGTASSSPGSSPS |
| | ASTGTGPGSSTPSGATGSPGASPGTSSTGSPGASPGTSSTGSPGSSTPSGATGSPGSSPSASTGTGP |
| | GASPGTSSTGSPGSSTPSGATGSPGTPGSGTASSSPGASPGTSSTGSPGTPGSGTASSSPGASPGTS |
| | STGSPGSSTPSGATGSPGSSTPSGATGSPGASPGTSSTGSPGSSPSASTGTGPGTPGSGTASSSPGT |
| | PGSGTASSSPGSSTPSGATGSPGTPGSGTASSSPGASPGTSSTGSPGSSTPSGATGSPGTPGSGTAS |
| | SSPGSSPSASTGTGPGSSTPSGATGSPGSSTPSGATGSPGSSPSASTGTGPGSSTPSGATGSPGTPG |
| | SGTASSSPGSSPSASTGTGPGTPGSGTASSSPGSSTPSGATGSPGSSPSASTGTGPGSSPSASTGTG |
| | PGTPGSGTASSSPGASP |
| AG1524 | GSSTPSGATGSPGTPGSGTASSSPGTPGSGTASSSPGASPGTSSTGSPGSSTPSGATGSPGTPGSGT |
| | ASSSPGSSTPSGATGSPGSSPSASTGTGPGSSTPSGATGSPGTPGSGTASSSPGTPGSGTASSSPGSS |
| | PSASTGTGPGSSTPSGATGSPGSSPSASTGTGPGTPGSGTASSSPGASPGTSSTGSPGSSTPSGATG |
| | SPGASPGTSSTGSPGTPGSGTASSSPGSSTPSGATGSPGTPGSGTASSSPGSSPSASTGTGPGASPG |
| | TSSTGSPGASPGTSSTGSPGTPGSGTASSSPGSSTPSGATGSPGSSTPSGATGSPGSSPSASTGTGP |
| | GSSPSASTGTGPGASPGTSSTGSPGTPGSGTASSSPGTPGSGTASSSPGSSPSASTGTGPGASPGTS |
| | STGSPGASPGTSSTGSPGTPGSGTASSSPGSSPSASTGTGPGASPGTSSTGSPGASPGTSSTGSPGS |
| | STPSGATGSPGSSPSASTGTGPGSSPSASTGTGPGTPGSGTASSSPGTPGSGTASSSPGASPGTSST |
| | GSPGSSTPSGATGSPGTPGSGTASSSPGASPGTSSTGSPGTPGSGTASSSPGTPGSGTASSSPGSST |
| | PSGATGSPGSSTPSGATGSPGTPGSGTASSSPGSSPSASTGTGPGSSTPSGATGSPGSSTPSGATGS |
| | PGSSPSASTGTGPGTPGSGTASSSPGSSPSASTGTGPGASPGTSSTGSPGSSPSASTGTGPGTPGSG |
| | TASSSPGASPGTSSTGSPGASPGTSSTGSPGTPGSGTASSSPGTPGSGTASSSPGSSTPSGATGSPG |
| | TPGSGTASSSPGASPGTSSTGSPGSSTPSGATGSPGSSTPSGATGSPGSSTPSGATGSPGTPGSGTA |
| | SSSPGSSPSASTGTGPGSSPSASTGTGPGSSPSASTGTGPGASPGTSSTGSPGASPGTSSTGSPGSSP |
| | SASTGTGPGTPGSGTASSSPGASPGTSSTGSPGSSTPSGATGSPGASPGTSSTGSPGASPGTSSTGS |
| | PGSSTPSGATGSPGTPG |
| AG1620 | GSSTPSGATGSPGSSTPSGATGSPGTPGSGTASSSPGSSPSASTGTGPGTPGSGTASSSPGASPGTS |
| | STGSPGSSTPSGATGSPGASPGTSSTGSPGTPGSGTASSSPGASPGTSSTGSPGSSPSASTGTGPGT |
| | PGSGTASSSPGASPGTSSTGSPGSSTPSGATGSPGSSPSASTGTGPGSSTPSGATGSPGASPGTSST |
| | GSPGASPGTSSTGSPGASPGTSSTGSPGSSTPSGATGSPGASPGTSSTGSPGTPGSGTASSSPGSST |
| | PSGATGSPGSSTPSGATGSPGSSPSASTGTGPGSSPSASTGTGPGSSTPSGATGSPGSSPSASTGTG |
| | PGSSPSASTGTGPGASPGTSSTGSPGASPGTSSTGSPGTPGSGTASSSPGSSPSASTGTGPGSSPSAS |
| | TGTGPGASPGTSSTGSPGSSPSASTGTGPGTPGSGTASSSPGSSPSASTGTGPGSSTPSGATGSPGA |
| | SPGTSSTGSPGSSTPSGATGSPGTPGSGTASSSPGSSPSASTGTGPGASPGTSSTGSPGSSTPSGAT |
| | GSPGASPGTSSTGSPGASPGTSSTGSPGTPGSGTASSSPGASPGTSSTGSPGASPGTSSTGSPGSST |
| | PSGATGSPGTPGSGTASSSPGSSPSASTGTGPGSSTPSGATGSPGTPGSGTASSSPGSSTPSGATGS |
| | PGSSTPSGATGSPGSSPSASTGTGPGSSPSASTGTGPGSSTPSGATGSPGASPGTSSTGSPGSSPSAS |
| | TGTGPGTPGSGTASSSPGASPGTSSTGSPGSSPSASTGTGPGSSTPSGATGSPGSSPSASTGTGPGS |
| | STPSGATGSPGSSPSASTGTGPGTPGSGTASSSPGTPGSGTASSSPGSSTPSGATGSPGSSTPSGAT |
| | GSPGTPGSGTASSSPGSSPSASTGTGPGSSTPSGATGSPGSSPSASTGTGPGSSPSASTGTGPGSST |
| | PSGATGSPGASPGTSSTGSPGASPGTSSTGSPGTPGSGTASSSPGTPGSGTASSSPGTPGSGTASSS |
| | PGSSTPSGATGSPGSST |
| AG1716 | GASPGTSSTGSPGSSPSASTGTGPGSSTPSGATGSPGSSPSASTGTGPGTPGSGTASSSPGSSTPSG |
| | ATGSPGSSTPSGATGSPGSSPSASTGTGPGSSTPSGATGSPGTPGSGTASSSPGSSPSASTGTGPGS |
| | STPSGATGSPGASPGTSSTGSPGSSPSASTGTGPGSSPSASTGTGPGTPGSGTASSSPGASPGTSST |
| | GSPGSSTPSGATGSPGSSPSASTGTGPGASPGTSSTGSPGSSTPSGATGSPGTPGSGTASSSPGSSPS |
| | ASTGTGPGSSPSASTGTGPGSSPSASTGTGPGTPGSGTASSSPGTPGSGTASSSPGSSTPSGATGSP |
| | GTPGSGTASSSPGSSPSASTGTGPGASPGTSSTGSPGASPGTSSTGSPGTPGSGTASSSPGSSPSAS |
| | TGTGPGASPGTSSTGSPGTPGSGTASSSPGSSTPSGATGSPGSSTPSGATGSPGASPGTSSTGSPGT |
| | PGSGTASSSPGSSPSASTGTGPGTPGSGTASSSPGASPGTSSTGSPGSSTPSGATGSPGASPGTSST |
| | GSPGASPGTSSTGSPGTPGSGTASSSPGTPGSGTASSSPGSSPSASTGTGPGSSPSASTGTGPGSSPS |
| | ASTGTGPGTPGSGTASSSPGASPGTSSTGSPGSSTPSGATGSPGASPGTSSTGSPGSSPSASTGTGP |
| | GASPGTSSTGSPGSSPSASTGTGPGASPGTSSTGSPGSSPSASTGTGPGSSPSASTGTGPGTPGSGT |
| | ASSSPGASPGTSSTGSPGTPGSGTASSSPGTPGSGTASSSPGASPGTSSTGSPGSSTPSGATGSPGSS |
| | TPSGATGSPGSSPSASTGTGPGSSTPSGATGSPGTPGSGTASSSPGSSPSASTGTGPGSSTPSGATG |
| | SPGSSTPSGATGSPGSSPSASTGTGPGSSPSASTGTGPGASPGTSSTGSPGTPGSGTASSSPGSSPSA |
| | STGTGPGSSTPSGATGSPGASPGTSSTGSPGASPGTSSTGSPGSSPSASTGTGPGASPGTSSTGSPG |
| | ASPGTSSTGSPGTPG |
| AG1812 | GSSTPSGATGSPGSSPSASTGTGPGASPGTSSTGSPGASPGTSSTGSPGTPGSGTASSSPGSSPSAS |
| | TGTGPGASPGTSSTGSPGSSTPSGATGSPGSSTPSGATGSPGASPGTSSTGSPGSSPSASTGTGPGT |
| | PGSGTASSSPGTPGSGTASSSPGASPGTSSTGSPGSSTPSGATGSPGSSTPSGATGSPGSSPSASTG |
| | TGPGTPGSGTASSSPGASPGTSSTGSPGSSTPSGATGSPGTPGSGTASSSPGSSPSASTGTGPGSST |
| | PSGATGSPGTPGSGTASSSPGSSPSASTGTGPGTPGSGTASSSPGASPGTSSTGSPGSSTPSGATGS |
| | PGSSTPSGATGSPGTPGSGTASSSPGSSTPSGATGSPGSSTPSGATGSPGASPGTSSTGSPGASPGT |
| | SSTGSPGASPGTSSTGSPGSSTPSGATGSPGTPGSGTASSSPGSSPSASTGTGPGTPGSGTASSSPG |
| | ASPGTSSTGSPGTPGSGTASSSPGASPGTSSTGSPGSSTPSGATGSPGSSPSASTGTGPGSSPSAST |
| | GTGPGTPGSGTASSSPGASPGTSSTGSPGASPGTSSTGSPGTPGSGTASSSPGASPGTSSTGSPGSS |
| | TPSGATGSPGSSTPSGATGSPGSSPSASTGTGPGASPGTSSTGSPGASPGTSSTGSPGTPGSGTASS |
| | SPGTPGSGTASSSPGSSTPSGATGSPGSSPSASTGTGPGSSPSASTGTGPGASPGTSSTGSPGASPG |
| | TSSTGSPGSSTPSGATGSPGSSPSASTGTGPGASPGTSSTGSPGSSTPSGATGSPGTPGSGTASSSP |
| | GSSPSASTGTGPGASPGTSSTGSPGSSTPSGATGSPGTPGSGTASSSPGSSTPSGATGSPGSSTPSG |
| | ATGSPGSSTPSGATGSPGSSPSASTGTGPGSSPSASTGTGPGSSTPSGATGSPGSSPSASTGTGPGS |
| | SPSASTGTGPGSSTPSGATGSPGASPGTSSTGSPGASPGTSSTGSPGTPGSGTASSSPGASPGTSST |
| | GSPGSSTPSGATGSPGASP |
| AG1908 | GSSPSASTGTGPGSSPSASTGTGPGSSPSASTGTGPGTPGSGTASSSPGSSPSASTGTGPGSSPSAS |
| | TGTGPGASPGTSSTGSPGSSPSASTGTGPGTPGSGTASSSPGTPGSGTASSSPGASPGTSSTGSPGT |
| | PGSGTASSSPGTPGSGTASSSPGSSPSASTGTGPGSSTPSGATGSPGSSPSASTGTGPGASPGTSST |
| | GSPGSSPSASTGTGPGASPGTSSTGSPGASPGTSSTGSPGTPGSGTASSSPGTPGSGTASSSPGASP |
| | GTSSTGSPGTPGSGTASSSPGTPGSGTASSSPGSSPSASTGTGPGSSTPSGATGSPGASPGTSSTGS |
| | PGSSTPSGATGSPGSSPSASTGTGPGSSPSASTGTGPGSSTPSGATGSPGASPGTSSTGSPGSSPSAS |
| | TGTGPGTPGSGTASSSPGASPGTSSTGSPGASPGTSSTGSPGTPGSGTASSSPGTPGSGTASSSPGS |
| | STPSGATGSPGTPGSGTASSSPGASPGTSSTGSPGSSTPSGATGSPGTPGSGTASSSPGSSPSASTG |
| | TGPGSSPSASTGTGPGSSTPSGATGSPGASPGTSSTGSPGASPGTSSTGSPGTPGSGTASSSPGTPG |
| | SGTASSSPGSSTPSGATGSPGSSPSASTGTGPGSSPSASTGTGPGASPGTSSTGSPGASPGTSSTGS |
| | PGSSTPSGATGSPGASPGTSSTGSPGASPGTSSTGSPGTPGSGTASSSPGTPGSGTASSSPGTPGSG |
| | TASSSPGSSTPSGATGSPGSSTPSGATGSPGSSPSASTGTGPGSSPSASTGTGPGSSTPSGATGSPG |
| | TPGSGTASSSPGSSPSASTGTGPGASPGTSSTGSPGSSTPSGATGSPGSSPSASTGTGPGSSTPSGA |
| | TGSPGASPGTSSTGSPGSSPSASTGTGPGTPGSGTASSSPGSSPSASTGTGPGSSTPSGATGSPGAS |
| | PGTSSTGSPGSSPSASTGTGPGTPGSGTASSSPGSSTPSGATGSPGSSTPSGATGSPGASPGTSSTG |
| | SPGSSPSASTGTGPGSSP |
| AG2004A | GSSPSASTGTGPGTPGSGTASSSPGSSTPSGATGSPGTPGSGTASSSPGSSTPSGATGSPGSSTPSG |
| | ATGSPGSSPSASTGTGPGSSPSASTGTGPGSSPSASTGTGPGTPGSGTASSSPGASPGTSSTGSPGS |
| | STPSGATGSPGTPGSGTASSSPGTPGSGTASSSPGSSTPSGATGSPGSSTPSGATGSPGSSPSASTG |
| | TGPGASPGTSSTGSPGASPGTSSTGSPGASPGTSSTGSPGASPGTSSTGSPGTPGSGTASSSPGTPG |
| | SGTASSSPGSSPSASTGTGPGSSTPSGATGSPGASPGTSSTGSPGSSTPSGATGSPGSSTPSGATGS |
| | PGSSPSASTGTGPGASPGTSSTGSPGTPGSGTASSSPGSSPSASTGTGPGSSPSASTGTGPGASPGT |
| | SSTGSPGASPGTSSTGSPGTPGSGTASSSPGASPGTSSTGSPGTPGSGTASSSPGTPGSGTASSSPG |
| | SSTPSGATGSPGTPGSGTASSSPGASPGTSSTGSPGSSTPSGATGSPGSSTPSGATGSPGSSPSAST |
| | GTGPGSSTPSGATGSPGSSTPSGATGSPGASPGTSSTGSPGSSPSASTGTGPGTPGSGTASSSPGAS |
| | PGTSSTGSPGSSPSASTGTGPGTPGSGTASSSPGSSPSASTGTGPGTPGSGTASSSPGASPGTSSTG |
| | SPGASPGTSSTGSPGTPGSGTASSSPGTPGSGTASSSPGSSTPSGATGSPGSSTPSGATGSPGTPGS |
| | GTASSSPGSSTPSGATGSPGSSTPSGATGSPGASPGTSSTGSPGSSPSASTGTGPGSSTPSGATGSP |
| | GSSPSASTGTGPGSSPSASTGTGPGASPGTSSTGSPGSSTPSGATGSPGASPGTSSTGSPGTPGSGT |
| | ASSSPGTPGSGTASSSPGSSPSASTGTGPGASPGTSSTGSPGSSTPSGATGSPGSSPSASTGTGPGSS |
| | PSASTGTGPGASPGTSSTGSPGTPGSGTASSSPGSSTPSGATGSPGTPGSGTASSSPGSSPSASTGT |
| | GPGSSPSASTGTGPGASP |
| AE72B | SPAGSPTSTEEGSPAGSPTSTEEGTSESATPESGPGTSTEPSEGSAPGTSESATPESGPGSEPATSGS |
| | ETPG |
| AE72C | TSESATPESGPGTSESATPESGPGSEPATSGSETPGSEPATSGSETPGSPAGSPTSTEEGTSTEPSEG |
| | SAPG |
| AE108A | TEEGTSESATPESGPGTSTEPSEGSAPGSPAGSPTSTEEGTSTEPSEGSAPGTSTEPSEGSAPGTSES |
| | ATPESGPGSEPATSGSETPGSEPATSGSETPGSPAGSPTS |
| AE108B | GSPAGSPTSTEEGTSTEPSEGSAPGTSESATPESGPGTSESATPESGPGTSESATPESGPGSEPATSG |
| | SETPGSEPATSGSETPGSPAGSPTSTEEGTSTEPSEGSAP |
| AE144A | STEPSEGSAPGSPAGSPTSTEEGTSTEPSEGSAPGTSESATPESGPGSEPATSGSETPGTSESATPES |
| | GPGSEPATSGSETPGTSESATPESGPGTSTEPSEGSAPGTSESATPESGPGSPAGSPTSTEEGSPAG |
| | SPTSTEEGS |
| AE144B | SEPATSGSETPGTSESATPESGPGSEPATSGSETPGTSESATPESGPGTSTEPSEGSAPGSPAGSPTS |
| | TEEGTSESATPESGPGSEPATSGSETPGTSESATPESGPGSPAGSPTSTEEGSPAGSPTSTEEGTSTE |
| | PSEGSAPG |
| AE180A | TSTEEGTSESATPESGPGSEPATSGSETPGTSESATPESGPGSPAGSPTSTEEGSPAGSPTSTEEGTS |
| | TEPSEGSAPGTSESATPESGPGTSESATPESGPGTSESATPESGPGSEPATSGSETPGSEPATSGSET |
| | PGSPAGSPTSTEEGTSTEPSEGSAPGTSTEPSEGSAPGSEPATS |
| AE216A | PESGPGTSTEPSEGSAPGSPAGSPTSTEEGTSESATPESGPGSEPATSGSETPGTSESATPESGPGSP |
| | AGSPTSTEEGSPAGSPTSTEEGTSTEPSEGSAPGTSESATPESGPGTSESATPESGPGTSESATPESG |
| | PGSEPATSGSETPGSEPATSGSETPGSPAGSPTSTEEGTSTEPSEGSAPGTSTEPSEGSAPGSEPATS |
| | GSETPGTSESAT |
| AE252A | ESGPGSEPATSGSETPGTSESATPESGPGTSTEPSEGSAPGSPAGSPTSTEEGTSESATPESGPGSEP |
| | ATSGSETPGTSESATPESGPGSPAGSPTSTEEGSPAGSPTSTEEGTSTEPSEGSAPGTSESATPESGP |
| | GTSESATPESGPGTSESATPESGPGSEPATSGSETPGSEPATSGSETPGSPAGSPTSTEEGTSTEPSE |
| | GSAPGTSTEPSEGSAPGSEPATSGSETPGTSESATPESGPGTSTEPSE |
| AE288A | TPESGPGTSTEPSEGSAPGTSESATPESGPGSEPATSGSETPGTSESATPESGPGSEPATSGSETPGT |
| | SESATPESGPGTSTEPSEGSAPGSPAGSPTSTEEGTSESATPESGPGSEPATSGSETPGTSESATPES |
| | GPGSPAGSPTSTEEGSPAGSPTSTEEGTSTEPSEGSAPGTSESATPESGPGTSESATPESGPGTSESA |
| | TPESGPGSEPATSGSETPGSEPATSGSETPGSPAGSPTSTEEGTSTEPSEGSAPGTSTEPSEGSAPGS |
| | EPATSGSETPGTSESA |
| AE324A | PESGPGSPAGSPTSTEEGSPAGSPTSTEEGSPAGSPTSTEEGTSESATPESGPGTSTEPSEGSAPGTS |
| | ESATPESGPGSEPATSGSETPGTSESATPESGPGSEPATSGSETPGTSESATPESGPGTSTEPSEGSA |
| | PGSPAGSPTSTEEGTSESATPESGPGSEPATSGSETPGTSESATPESGPGSPAGSPTSTEEGSPAGSP |
| | TSTEEGTSTEPSEGSAPGTSESATPESGPGTSESATPESGPGTSESATPESGPGSEPATSGSETPGSE |
| | PATSGSETPGSPAGSPTSTEEGTSTEPSEGSAPGTSTEPSEGSAPGSEPATS |
| AE360A | PESGPGTSTEPSEGSAPGTSESATPESGPGSPAGSPTSTEEGSPAGSPTSTEEGSPAGSPTSTEEGTS |
| | ESATPESGPGTSTEPSEGSAPGTSESATPESGPGSEPATSGSETPGTSESATPESGPGSEPATSGSET |
| | PGTSESATPESGPGTSTEPSEGSAPGSPAGSPTSTEEGTSESATPESGPGSEPATSGSETPGTSESAT |
| | PESGPGSPAGSPTSTEEGSPAGSPTSTEEGTSTEPSEGSAPGTSESATPESGPGTSESATPESGPGTS |
| | ESATPESGPGSEPATSGSETPGSEPATSGSETPGSPAGSPTSTEEGTSTEPSEGSAPGTSTEPSEGSA |
| | PGSEPATSGSETPGTSESAT |
| AE396A | PESGPGSEPATSGSETPGTSESATPESGPGTSTEPSEGSAPGTSESATPESGPGSPAGSPTSTEEGSP |
| | AGSPTSTEEGSPAGSPTSTEEGTSESATPESGPGTSTEPSEGSAPGTSESATPESGPGSEPATSGSET |
| | PGTSESATPESGPGSEPATSGSETPGTSESATPESGPGTSTEPSEGSAPGSPAGSPTSTEEGTSESAT |
| | PESGPGSEPATSGSETPGTSESATPESGPGSPAGSPTSTEEGSPAGSPTSTEEGTSTEPSEGSAPGTS |
| | ESATPESGPGTSESATPESGPGTSESATPESGPGSEPATSGSETPGSEPATSGSETPGSPAGSPTSTE |
| | EGTSTEPSEGSAPGTSTEPS |
| AE432A | EGSAPGSPAGSPTSTEEGTSTEPSEGSAPGTSESATPESGPGSEPATSGSETPGTSESATPESGPGSE |
| | PATSGSETPGTSESATPESGPGTSTEPSEGSAPGTSESATPESGPGSPAGSPTSTEEGSPAGSPTSTE |
| | EGSPAGSPTSTEEGTSESATPESGPGTSTEPSEGSAPGTSESATPESGPGSEPATSGSETPGTSESAT |
| | PESGPGSEPATSGSETPGTSESATPESGPGTSTEPSEGSAPGSPAGSPTSTEEGTSESATPESGPGSE |
| | PATSGSETPGTSESATPESGPGSPAGSPTSTEEGSPAGSPTSTEEGTSTEPSEGSAPGTSESATPESG |
| | PGTSESATPESGPGTSESATPESGPGSEPATSGSETPGSEPATSGSETPGSPAGSPTSTEEGTSTEPS |
| | EGSAPGTSTEPSEGSAPGSEPATS |
| AE468A | EGSAPGTSTEPSEGSAPGTSTEPSEGSAPGSPAGSPTSTEEGTSTEPSEGSAPGTSESATPESGPGSE |
| | PATSGSETPGTSESATPESGPGSEPATSGSETPGTSESATPESGPGTSTEPSEGSAPGTSESATPESG |
| | PGSPAGSPTSTEEGSPAGSPTSTEEGSPAGSPTSTEEGTSESATPESGPGTSTEPSEGSAPGTSESAT |
| | PESGPGSEPATSGSETPGTSESATPESGPGSEPATSGSETPGTSESATPESGPGTSTEPSEGSAPGSP |
| | AGSPTSTEEGTSESATPESGPGSEPATSGSETPGTSESATPESGPGSPAGSPTSTEEGSPAGSPTSTE |
| | EGTSTEPSEGSAPGTSESATPESGPGTSESATPESGPGTSESATPESGPGSEPATSGSETPGSEPATS |
| | GSETPGSPAGSPTSTEEGTSTEPSEGSAPGTSTEPSEGSAPGSEPATSGSETPGTSESAT |
| AE504A | EGSAPGTSTEPSEGSAPGTSTEPSEGSAPGTSTEPSEGSAPGTSTEPSEGSAPGSPAGSPTSTEEGTS |
| | TEPSEGSAPGTSESATPESGPGSEPATSGSETPGTSESATPESGPGSEPATSGSETPGTSESATPESG |
| | PGTSTEPSEGSAPGTSESATPESGPGSPAGSPTSTEEGSPAGSPTSTEEGSPAGSPTSTEEGTSESAT |
| | PESGPGTSTEPSEGSAPGTSESATPESGPGSEPATSGSETPGTSESATPESGPGSEPATSGSETPGTS |
| | ESATPESGPGTSTEPSEGSAPGSPAGSPTSTEEGTSESATPESGPGSEPATSGSETPGTSESATPESG |
| | PGSPAGSPTSTEEGSPAGSPTSTEEGTSTEPSEGSAPGTSESATPESGPGTSESATPESGPGTSESAT |
| | PESGPGSEPATSGSETPGSEPATSGSETPGSPAGSPTSTEEGTSTEPSEGSAPGTSTEPSEGSAPGSE |
| | PATSGSETPGTSESATPESGPGTSTEPS |
| AE540A | TPESGPGSPAGSPTSTEEGTSESATPESGPGSEPATSGSETPGTSESATPESGPGTSTEPSEGSAPGT |
| | STEPSEGSAPGTSTEPSEGSAPGTSTEPSEGSAPGTSTEPSEGSAPGTSTEPSEGSAPGSPAGSPTST |
| | EEGTSTEPSEGSAPGTSESATPESGPGSEPATSGSETPGTSESATPESGPGSEPATSGSETPGTSESA |
| | TPESGPGTSTEPSEGSAPGTSESATPESGPGSPAGSPTSTEEGSPAGSPTSTEEGSPAGSPTSTEEGT |
| | SESATPESGPGTSTEPSEGSAPGTSESATPESGPGSEPATSGSETPGTSESATPESGPGSEPATSGSE |
| | TPGTSESATPESGPGTSTEPSEGSAPGSPAGSPTSTEEGTSESATPESGPGSEPATSGSETPGTSESA |
| | TPESGPGSPAGSPTSTEEGSPAGSPTSTEEGTSTEPSEGSAPGTSESATPESGPGTSESATPESGPGT |
| | SESATPESGPGSEPATSGSETPGSEPATSGSETPGSPAGSPTSTEEGTSTEPSEGSAPGTSTEP |
| AE576A | TPESGPGTSESATPESGPGSPAGSPTSTEEGTSESATPESGPGSEPATSGSETPGTSESATPESGPGT |
| | STEPSEGSAPGTSTEPSEGSAPGTSTEPSEGSAPGTSTEPSEGSAPGTSTEPSEGSAPGTSTEPSEGS |
| | APGSPAGSPTSTEEGTSTEPSEGSAPGTSESATPESGPGSEPATSGSETPGTSESATPESGPGSEPAT |
| | SGSETPGTSESATPESGPGTSTEPSEGSAPGTSESATPESGPGSPAGSPTSTEEGSPAGSPTSTEEGS |
| | PAGSPTSTEEGTSESATPESGPGTSTEPSEGSAPGTSESATPESGPGSEPATSGSETPGTSESATPES |
| | GPGSEPATSGSETPGTSESATPESGPGTSTEPSEGSAPGSPAGSPTSTEEGTSESATPESGPGSEPAT |
| | SGSETPGTSESATPESGPGSPAGSPTSTEEGSPAGSPTSTEEGTSTEPSEGSAPGTSESATPESGPGT |
| | SESATPESGPGTSESATPESGPGSEPATSGSETPGSEPATSGSETPGSPAGSPTSTEEGTSTEPSEGS |
| | APGTSTEPSEGSAPGSEPATSGSETPGTSESA |
| AE612A | GSETPGTSTEPSEGSAPGTSTEPSEGSAPGTSESATPESGPGTSESATPESGPGSPAGSPTSTEEGTS |
| | ESATPESGPGSEPATSGSETPGTSESATPESGPGTSTEPSEGSAPGTSTEPSEGSAPGTSTEPSEGSA |
| | PGTSTEPSEGSAPGTSTEPSEGSAPGTSTEPSEGSAPGSPAGSPTSTEEGTSTEPSEGSAPGTSESAT |
| | PESGPGSEPATSGSETPGTSESATPESGPGSEPATSGSETPGTSESATPESGPGTSTEPSEGSAPGTS |
| | ESATPESGPGSPAGSPTSTEEGSPAGSPTSTEEGSPAGSPTSTEEGTSESATPESGPGTSTEPSEGSA |
| | PGTSESATPESGPGSEPATSGSETPGTSESATPESGPGSEPATSGSETPGTSESATPESGPGTSTEPS |
| | EGSAPGSPAGSPTSTEEGTSESATPESGPGSEPATSGSETPGTSESATPESGPGSPAGSPTSTEEGSP |
| | AGSPTSTEEGTSTEPSEGSAPGTSESATPESGPGTSESATPESGPGTSESATPESGPGSEPATSGSET |
| | PGSEPATSGSETPGSPAGSPTSTEEGTSTEPSEGSAPGTSTEPSEGSAPGSEPATSGSETPGTSESAT |
| AE648A | PESGPGTSTEPSEGSAPGTSESATPESGPGSEPATSGSETPGTSTEPSEGSAPGTSTEPSEGSAPGTS |
| | ESATPESGPGTSESATPESGPGSPAGSPTSTEEGTSESATPESGPGSEPATSGSETPGTSESATPESG |
| | PGTSTEPSEGSAPGTSTEPSEGSAPGTSTEPSEGSAPGTSTEPSEGSAPGTSTEPSEGSAPGTSTEPS |
| | EGSAPGSPAGSPTSTEEGTSTEPSEGSAPGTSESATPESGPGSEPATSGSETPGTSESATPESGPGSE |
| | PATSGSETPGTSESATPESGPGTSTEPSEGSAPGTSESATPESGPGSPAGSPTSTEEGSPAGSPTSTE |
| | EGSPAGSPTSTEEGTSESATPESGPGTSTEPSEGSAPGTSESATPESGPGSEPATSGSETPGTSESAT |
| | PESGPGSEPATSGSETPGTSESATPESGPGTSTEPSEGSAPGSPAGSPTSTEEGTSESATPESGPGSE |
| | PATSGSETPGTSESATPESGPGSPAGSPTSTEEGSPAGSPTSTEEGTSTEPSEGSAPGTSESATPESG |
| | PGTSESATPESGPGTSESATPESGPGSEPATSGSETPGSEPATSGSETPGSPAGSPTSTEEGTSTEPS |
| | EGSAPGTSTEPSEGSAPGSEPATSGSETPGTSESAT |
| AE684A | EGSAPGSPAGSPTSTEEGTSTEPSEGSAPGTSTEPSEGSAPGTSESATPESGPGTSTEPSEGSAPGTS |
| | ESATPESGPGSEPATSGSETPGTSTEPSEGSAPGTSTEPSEGSAPGTSESATPESGPGTSESATPESG |
| | PGSPAGSPTSTEEGTSESATPESGPGSEPATSGSETPGTSESATPESGPGTSTEPSEGSAPGTSTEPS |
| | EGSAPGTSTEPSEGSAPGTSTEPSEGSAPGTSTEPSEGSAPGTSTEPSEGSAPGSPAGSPTSTEEGTS |
| | TEPSEGSAPGTSESATPESGPGSEPATSGSETPGTSESATPESGPGSEPATSGSETPGTSESATPESG |
| | PGTSTEPSEGSAPGTSESATPESGPGSPAGSPTSTEEGSPAGSPTSTEEGSPAGSPTSTEEGTSESAT |
| | PESGPGTSTEPSEGSAPGTSESATPESGPGSEPATSGSETPGTSESATPESGPGSEPATSGSETPGTS |
| | ESATPESGPGTSTEPSEGSAPGSPAGSPTSTEEGTSESATPESGPGSEPATSGSETPGTSESATPESG |
| | PGSPAGSPTSTEEGSPAGSPTSTEEGTSTEPSEGSAPGTSESATPESGPGTSESATPESGPGTSESAT |
| | PESGPGSEPATSGSETPGSEPATSGSETPGSPAGSPTSTEEGTSTEPSEGSAPGTSTEPSEGSAPGSE |
| | PATS |
| AE720A | TSGSETPGSEPATSGSETPGSPAGSPTSTEEGTSESATPESGPGTSTEPSEGSAPGTSTEPSEGSAPG |
| | SPAGSPTSTEEGTSTEPSEGSAPGTSTEPSEGSAPGTSESATPESGPGTSTEPSEGSAPGTSESATPE |
| | SGPGSEPATSGSETPGTSTEPSEGSAPGTSTEPSEGSAPGTSESATPESGPGTSESATPESGPGSPA |
| | GSPTSTEEGTSESATPESGPGSEPATSGSETPGTSESATPESGPGTSTEPSEGSAPGTSTEPSEGSAP |
| | GTSTEPSEGSAPGTSTEPSEGSAPGTSTEPSEGSAPGTSTEPSEGSAPGSPAGSPTSTEEGTSTEPSE |
| | GSAPGTSESATPESGPGSEPATSGSETPGTSESATPESGPGSEPATSGSETPGTSESATPESGPGTST |
| | EPSEGSAPGTSESATPESGPGSPAGSPTSTEEGSPAGSPTSTEEGSPAGSPTSTEEGTSESATPESGP |
| | GTSTEPSEGSAPGTSESATPESGPGSEPATSGSETPGTSESATPESGPGSEPATSGSETPGTSESATP |
| | ESGPGTSTEPSEGSAPGSPAGSPTSTEEGTSESATPESGPGSEPATSGSETPGTSESATPESGPGSPA |
| | GSPTSTEEGSPAGSPTSTEEGTSTEPSEGSAPGTSESATPESGPGTSESATPESGPGTSESATPESGP |
| | GSEPATSGSETPGSEPATSGSETPGSPAGSPTSTEEGTSTE |
| AE756A | TSGSETPGSEPATSGSETPGSPAGSPTSTEEGTSESATPESGPGTSTEPSEGSAPGTSTEPSEGSAPG |
| | SPAGSPTSTEEGTSTEPSEGSAPGTSTEPSEGSAPGTSESATPESGPGTSTEPSEGSAPGTSESATPE |
| | SGPGSEPATSGSETPGTSTEPSEGSAPGTSTEPSEGSAPGTSESATPESGPGTSESATPESGPGSPA |
| | GSPTSTEEGTSESATPESGPGSEPATSGSETPGTSESATPESGPGTSTEPSEGSAPGTSTEPSEGSAP |
| | GTSTEPSEGSAPGTSTEPSEGSAPGTSTEPSEGSAPGTSTEPSEGSAPGSPAGSPTSTEEGTSTEPSE |
| | GSAPGTSESATPESGPGSEPATSGSETPGTSESATPESGPGSEPATSGSETPGTSESATPESGPGTST |
| | EPSEGSAPGTSESATPESGPGSPAGSPTSTEEGSPAGSPTSTEEGSPAGSPTSTEEGTSESATPESGP |
| | GTSTEPSEGSAPGTSESATPESGPGSEPATSGSETPGTSESATPESGPGSEPATSGSETPGTSESATP |
| | ESGPGTSTEPSEGSAPGSPAGSPTSTEEGTSESATPESGPGSEPATSGSETPGTSESATPESGPGSPA |
| | GSPTSTEEGSPAGSPTSTEEGTSTEPSEGSAPGTSESATPESGPGTSESATPESGPGTSESATPESGP |
| | GSEPATSGSETPGSEPATSGSETPGSPAGSPTSTEEGTSTEPSEGSAPGTSTEPSEGSAPGSEPATSG |
| | SETPGTSES |
| AE792A | EGSAPGTSESATPESGPGSEPATSGSETPGSEPATSGSETPGSPAGSPTSTEEGTSESATPESGPGTS |
| | TEPSEGSAPGTSTEPSEGSAPGSPAGSPTSTEEGTSTEPSEGSAPGTSTEPSEGSAPGTSESATPESG |
| | PGTSTEPSEGSAPGTSESATPESGPGSEPATSGSETPGTSTEPSEGSAPGTSTEPSEGSAPGTSESAT |
| | PESGPGTSESATPESGPGSPAGSPTSTEEGTSESATPESGPGSEPATSGSETPGTSESATPESGPGTS |
| | TEPSEGSAPGTSTEPSEGSAPGTSTEPSEGSAPGTSTEPSEGSAPGTSTEPSEGSAPGTSTEPSEGSA |
| | PGSPAGSPTSTEEGTSTEPSEGSAPGTSESATPESGPGSEPATSGSETPGTSESATPESGPGSEPATS |
| | GSETPGTSESATPESGPGTSTEPSEGSAPGTSESATPESGPGSPAGSPTSTEEGSPAGSPTSTEEGSP |
| | AGSPTSTEEGTSESATPESGPGTSTEPSEGSAPGTSESATPESGPGSEPATSGSETPGTSESATPESG |
| | PGSEPATSGSETPGTSESATPESGPGTSTEPSEGSAPGSPAGSPTSTEEGTSESATPESGPGSEPATS |
| | GSETPGTSESATPESGPGSPAGSPTSTEEGSPAGSPTSTEEGTSTEPSEGSAPGTSESATPESGPGTS |
| | ESATPESGPGTSESATPESGPGSEPATSGSETPGSEPATSGSETPGSPAGSPTSTEEGTSTEPSEGSA |
| | PGTSTEPSEGSAPGSEPATSGSETPGTSESATPESGPGTSTEPS |
| AE828A | PESGPGTSTEPSEGSAPGSPAGSPTSTEEGTSTEPSEGSAPGTSTEPSEGSAPGTSESATPESGPGSE |
| | PATSGSETPGSEPATSGSETPGSPAGSPTSTEEGTSESATPESGPGTSTEPSEGSAPGTSTEPSEGSA |
| | PGSPAGSPTSTEEGTSTEPSEGSAPGTSTEPSEGSAPGTSESATPESGPGTSTEPSEGSAPGTSESAT |
| | PESGPGSEPATSGSETPGTSTEPSEGSAPGTSTEPSEGSAPGTSESATPESGPGTSESATPESGPGSP |
| | AGSPTSTEEGTSESATPESGPGSEPATSGSETPGTSESATPESGPGTSTEPSEGSAPGTSTEPSEGSA |
| | PGTSTEPSEGSAPGTSTEPSEGSAPGTSTEPSEGSAPGTSTEPSEGSAPGSPAGSPTSTEEGTSTEPS |
| | EGSAPGTSESATPESGPGSEPATSGSETPGTSESATPESGPGSEPATSGSETPGTSESATPESGPGTS |
| | TEPSEGSAPGTSESATPESGPGSPAGSPTSTEEGSPAGSPTSTEEGSPAGSPTSTEEGTSESATPESG |
| | PGTSTEPSEGSAPGTSESATPESGPGSEPATSGSETPGTSESATPESGPGSEPATSGSETPGTSESAT |
| | PESGPGTSTEPSEGSAPGSPAGSPTSTEEGTSESATPESGPGSEPATSGSETPGTSESATPESGPGSP |
| | AGSPTSTEEGSPAGSPTSTEEGTSTEPSEGSAPGTSESATPESGPGTSESATPESGPGTSESATPESG |
| | PGSEPATSGSETPGSEPATSGSETPGSPAGSPTSTEEGTSTEPSEGSAPGTSTEPSEGSAPGSEPATS |
| | GSETPGTSESAT |
| AG72A | GPGSSPSASTGTGPGTPGSGTASSSPGSSTPSGATGSPGSSPSASTGTGPGASPGTSSTGSPGTPGS |
| | GTASS |
| AG72B | GSSTPSGATGSPGSSTPSGATGSPGSSPSASTGTGPGSSPSASTGTGPGASPGTSSTGSPGTPGSGT |
| | ASSSP |
| AG72C | SPSASTGTGPGASPGTSSTGSPGSSPSASTGTGPGTPGSGTASSSPGSSTPSGATGSPGSSTPSGAT |
| | GSPGA |
| AG108A | SASTGTGPGSSPSASTGTGPGTPGSGTASSSPGSSTPSGATGSPGSSPSASTGTGPGASPGTSSTGS |
| | PGTPGSGTASSSPGSSTPSGATGSPGTPGSGTASSSPGASP |
| AG108B | PGTPGSGTASSSPGSSTPSGATGSPGTPGSGTASSSPGSSTPSGATGSPGSSTPSGATGSPGSSPSAS |
| | TGTGPGSSPSASTGTGPGASPGTSSTGSPGTPGSGTASSS |
| AG144A | PGSSPSASTGTGPGSSPSASTGTGPGTPGSGTASSSPGSSTPSGATGSPGSSPSASTGTGPGASPGT |
| | SSTGSPGTPGSGTASSSPGSSTPSGATGSPGTPGSGTASSSPGASPGTSSTGSPGASPGTSSTGSPG |
| | TPGSGTASSS |
| AG144B | PSGATGSPGTPGSGTASSSPGSSTPSGATGSPGSSTPSGATGSPGSSPSASTGTGPGSSPSASTGTG |
| | PGASPGTSSTGSPGTPGSGTASSSPGSSTPSGATGSPGSSPSASTGTGPGSSPSASTGTGPGASPGT |
| | SSTGSPGASP |
| AG180A | TSSTGSPGSSPSASTGTGPGSSPSASTGTGPGTPGSGTASSSPGSSTPSGATGSPGSSPSASTGTGP |
| | GASPGTSSTGSPGTPGSGTASSSPGSSTPSGATGSPGTPGSGTASSSPGASPGTSSTGSPGASPGTS |
| | STGSPGTPGSGTASSSPGSSTPSGATGSPGASPGTSSTGSPGTPGS |
| AG216A | TGTGPGSSPSASTGTGPGTPGSGTASSSPGSSTPSGATGSPGSSPSASTGTGPGASPGTSSTGSPGT |
| | PGSGTASSSPGSSTPSGATGSPGTPGSGTASSSPGASPGTSSTGSPGASPGTSSTGSPGTPGSGTAS |
| | SSPGSSTPSGATGSPGASPGTSSTGSPGTPGSGTASSSPGSSTPSGATGSPGSSPSASTGTGPGSSPS |
| | ASTGTGPGSSTPSG |
| AG252A | TSSTGSPGSSPSASTGTGPGSSPSASTGTGPGTPGSGTASSSPGSSTPSGATGSPGSSPSASTGTGP |
| | GASPGTSSTGSPGTPGSGTASSSPGSSTPSGATGSPGTPGSGTASSSPGASPGTSSTGSPGASPGTS |
| | STGSPGTPGSGTASSSPGSSTPSGATGSPGASPGTSSTGSPGTPGSGTASSSPGSSTPSGATGSPGS |
| | SPSASTGTGPGSSPSASTGTGPGSSTPSGATGSPGSSTPSGATGSPGASPG |
| AG288A | TSSTGSPGSSPSASTGTGPGSSPSASTGTGPGTPGSGTASSSPGSSTPSGATGSPGSSPSASTGTGP |
| | GASPGTSSTGSPGTPGSGTASSSPGSSTPSGATGSPGTPGSGTASSSPGASPGTSSTGSPGASPGTS |
| | STGSPGTPGSGTASSSPGSSTPSGATGSPGASPGTSSTGSPGTPGSGTASSSPGSSTPSGATGSPGS |
| | SPSASTGTGPGSSPSASTGTGPGSSTPSGATGSPGSSTPSGATGSPGASPGTSSTGSPGASPGTSST |
| | GSPGASPGTSSTGSPGTPGS |
| AG324A | TSSTGSPGTPGSGTASSSPGASPGTSSTGSPGASPGTSSTGSPGASPGTSSTGSPGASPGTSSTGSP |
| | GTPGSGTASSSPGSSTPSGATGSPGTPGSGTASSSPGSSTPSGATGSPGTPGSGTASSSPGSSTPSG |
| | ATGSPGSSTPSGATGSPGSSPSASTGTGPGSSPSASTGTGPGASPGTSSTGSPGTPGSGTASSSPGS |
| | STPSGATGSPGSSPSASTGTGPGSSPSASTGTGPGASPGTSSTGSPGASPGTSSTGSPGSSTPSGAT |
| | GSPGSSPSASTGTGPGASPGTSSTGSPGSSPSASTGTGPGTPGSGTASSSPGSSTP |
| AG360A | TSSTGSPGASPGTSSTGSPGTPGSGTASSSPGASPGTSSTGSPGASPGTSSTGSPGASPGTSSTGSP |
| | GASPGTSSTGSPGTPGSGTASSSPGSSTPSGATGSPGTPGSGTASSSPGSSTPSGATGSPGTPGSGT |
| | ASSSPGSSTPSGATGSPGSSTPSGATGSPGSSPSASTGTGPGSSPSASTGTGPGASPGTSSTGSPGT |
| | PGSGTASSSPGSSTPSGATGSPGSSPSASTGTGPGSSPSASTGTGPGASPGTSSTGSPGASPGTSST |
| | GSPGSSTPSGATGSPGSSPSASTGTGPGASPGTSSTGSPGSSPSASTGTGPGTPGSGTASSSPGSST |
| | PSGATGSPGSSTPSGATGSPGASPG |
| AG396A | GATGSPGSSTPSGATGSPGSSPSASTGTGPGASPGTSSTGSPGASPGTSSTGSPGTPGSGTASSSPG |
| | ASPGTSSTGSPGASPGTSSTGSPGASPGTSSTGSPGASPGTSSTGSPGTPGSGTASSSPGSSTPSGA |
| | TGSPGTPGSGTASSSPGSSTPSGATGSPGTPGSGTASSSPGSSTPSGATGSPGSSTPSGATGSPGSS |
| | PSASTGTGPGSSPSASTGTGPGASPGTSSTGSPGTPGSGTASSSPGSSTPSGATGSPGSSPSASTGT |
| | GPGSSPSASTGTGPGASPGTSSTGSPGASPGTSSTGSPGSSTPSGATGSPGSSPSASTGTGPGASPG |
| | TSSTGSPGSSPSASTGTGPGTPGSGTASSSPGSSTPSGATGSPGSSTPSGATGSPGASPGT |
| AG432A | GATGSPGSSTPSGATGSPGASPGTSSTGSPGTPGSGTASSSPGSSTPSGATGSPGSSTPSGATGSPG |
| | SSTPSGATGSPGSSPSASTGTGPGASPGTSSTGSPGASPGTSSTGSPGTPGSGTASSSPGASPGTSS |
| | TGSPGASPGTSSTGSPGASPGTSSTGSPGASPGTSSTGSPGTPGSGTASSSPGSSTPSGATGSPGTP |
| | GSGTASSSPGSSTPSGATGSPGTPGSGTASSSPGSSTPSGATGSPGSSTPSGATGSPGSSPSASTGT |
| | GPGSSPSASTGTGPGASPGTSSTGSPGTPGSGTASSSPGSSTPSGATGSPGSSPSASTGTGPGSSPS |
| | ASTGTGPGASPGTSSTGSPGASPGTSSTGSPGSSTPSGATGSPGSSPSASTGTGPGASPGTSSTGSP |
| | GSSPSASTGTGPGTPGSGTASSSPGSSTPS |
| AG468A | TSSTGSPGSSPSASTGTGPGTPGSGTASSSPGASPGTSSTGSPGASPGTSSTGSPGASPGTSSTGSP |
| | GSSTPSGATGSPGSSTPSGATGSPGASPGTSSTGSPGTPGSGTASSSPGSSTPSGATGSPGSSTPSG |
| | ATGSPGSSTPSGATGSPGSSPSASTGTGPGASPGTSSTGSPGASPGTSSTGSPGTPGSGTASSSPGA |
| | SPGTSSTGSPGASPGTSSTGSPGASPGTSSTGSPGASPGTSSTGSPGTPGSGTASSSPGSSTPSGAT |
| | GSPGTPGSGTASSSPGSSTPSGATGSPGTPGSGTASSSPGSSTPSGATGSPGSSTPSGATGSPGSSPS |
| | ASTGTGPGSSPSASTGTGPGASPGTSSTGSPGTPGSGTASSSPGSSTPSGATGSPGSSPSASTGTGP |
| | GSSPSASTGTGPGASPGTSSTGSPGASPGTSSTGSPGSSTPSGATGSPGSSPSASTGTGPGASPG |
| AG504A | TSSTGSPGSSPSASTGTGPGTPGSGTASSSPGASPGTSSTGSPGASPGTSSTGSPGASPGTSSTGSP |
| | GSSTPSGATGSPGSSTPSGATGSPGASPGTSSTGSPGTPGSGTASSSPGSSTPSGATGSPGSSTPSG |
| | ATGSPGSSTPSGATGSPGSSPSASTGTGPGASPGTSSTGSPGASPGTSSTGSPGTPGSGTASSSPGA |
| | SPGTSSTGSPGASPGTSSTGSPGASPGTSSTGSPGASPGTSSTGSPGTPGSGTASSSPGSSTPSGAT |
| | GSPGTPGSGTASSSPGSSTPSGATGSPGTPGSGTASSSPGSSTPSGATGSPGSSTPSGATGSPGSSPS |
| | ASTGTGPGSSPSASTGTGPGASPGTSSTGSPGTPGSGTASSSPGSSTPSGATGSPGSSPSASTGTGP |
| | GSSPSASTGTGPGASPGTSSTGSPGASPGTSSTGSPGSSTPSGATGSPGSSPSASTGTGPGASPGTS |
| | STGSPGSSPSASTGTGPGTPGSGTASSSPGSSTP |
| AG540A | TSSTGSPGASPGTSSTGSPGSSPSASTGTGPGTPGSGTASSSPGASPGTSSTGSPGASPGTSSTGSP |
| | GASPGTSSTGSPGSSTPSGATGSPGSSTPSGATGSPGASPGTSSTGSPGTPGSGTASSSPGSSTPSG |
| | ATGSPGSSTPSGATGSPGSSTPSGATGSPGSSPSASTGTGPGASPGTSSTGSPGASPGTSSTGSPGT |
| | PGSGTASSSPGASPGTSSTGSPGASPGTSSTGSPGASPGTSSTGSPGASPGTSSTGSPGTPGSGTAS |
| | SSPGSSTPSGATGSPGTPGSGTASSSPGSSTPSGATGSPGTPGSGTASSSPGSSTPSGATGSPGSSTP |
| | SGATGSPGSSPSASTGTGPGSSPSASTGTGPGASPGTSSTGSPGTPGSGTASSSPGSSTPSGATGSP |
| | GSSPSASTGTGPGSSPSASTGTGPGASPGTSSTGSPGASPGTSSTGSPGSSTPSGATGSPGSSPSAS |
| | TGTGPGASPGTSSTGSPGSSPSASTGTGPGTPGSGTASSSPGSSTPSGATGSPGSSTPSGATGSPGA |
| | SPG |
| AG576A | TSSTGSPGTPGSGTASSSPGASPGTSSTGSPGASPGTSSTGSPGASPGTSSTGSPGSSPSASTGTGP |
| | GTPGSGTASSSPGASPGTSSTGSPGASPGTSSTGSPGASPGTSSTGSPGSSTPSGATGSPGSSTPSG |
| | ATGSPGASPGTSSTGSPGTPGSGTASSSPGSSTPSGATGSPGSSTPSGATGSPGSSTPSGATGSPGS |
| | SPSASTGTGPGASPGTSSTGSPGASPGTSSTGSPGTPGSGTASSSPGASPGTSSTGSPGASPGTSST |
| | GSPGASPGTSSTGSPGASPGTSSTGSPGTPGSGTASSSPGSSTPSGATGSPGTPGSGTASSSPGSST |
| | PSGATGSPGTPGSGTASSSPGSSTPSGATGSPGSSTPSGATGSPGSSPSASTGTGPGSSPSASTGTG |
| | PGASPGTSSTGSPGTPGSGTASSSPGSSTPSGATGSPGSSPSASTGTGPGSSPSASTGTGPGASPGT |
| | SSTGSPGASPGTSSTGSPGSSTPSGATGSPGSSPSASTGTGPGASPGTSSTGSPGSSPSASTGTGPG |
| | TPGSGTASSSPGSSTPSGATGSPGSSTPSGATGSPGASPG |
| AG612A | STGSPGTPGSGTASSSPGSSTPSGATGSPGASPGTSSTGSPGTPGSGTASSSPGSSTPSGATGSPGS |
| | SPSASTGTGPGSSPSASTGTGPGSSTPSGATGSPGSSTPSGATGSPGASPGTSSTGSPGASPGTSST |
| | GSPGASPGTSSTGSPGTPGSGTASSSPGASPGTSSTGSPGASPGTSSTGSPGASPGTSSTGSPGSSPS |
| | ASTGTGPGTPGSGTASSSPGASPGTSSTGSPGASPGTSSTGSPGASPGTSSTGSPGSSTPSGATGSP |
| | GSSTPSGATGSPGASPGTSSTGSPGTPGSGTASSSPGSSTPSGATGSPGSSTPSGATGSPGSSTPSG |
| | ATGSPGSSPSASTGTGPGASPGTSSTGSPGASPGTSSTGSPGTPGSGTASSSPGASPGTSSTGSPGA |
| | SPGTSSTGSPGASPGTSSTGSPGASPGTSSTGSPGTPGSGTASSSPGSSTPSGATGSPGTPGSGTAS |
| | SSPGSSTPSGATGSPGTPGSGTASSSPGSSTPSGATGSPGSSTPSGATGSPGSSPSASTGTGPGSSPS |
| | ASTGTGPGASPGTSSTGSPGTPGSGTASSSPGSSTPSGATGSPGSSPSASTGTGPGSSPSASTGTGP |
| | GASPGTS |
| AG648A | GTASSSPGSSTPSGATGSPGSSPSASTGTGPGSSPSASTGTGPGSSTPSGATGSPGSSTPSGATGSP |
| | GASPGTSSTGSPGASPGTSSTGSPGASPGTSSTGSPGTPGSGTASSSPGASPGTSSTGSPGASPGTS |
| | STGSPGASPGTSSTGSPGSSPSASTGTGPGTPGSGTASSSPGASPGTSSTGSPGASPGTSSTGSPGA |
| | SPGTSSTGSPGSSTPSGATGSPGSSTPSGATGSPGASPGTSSTGSPGTPGSGTASSSPGSSTPSGAT |
| | GSPGSSTPSGATGSPGSSTPSGATGSPGSSPSASTGTGPGASPGTSSTGSPGASPGTSSTGSPGTPG |
| | SGTASSSPGASPGTSSTGSPGASPGTSSTGSPGASPGTSSTGSPGASPGTSSTGSPGTPGSGTASSS |
| | PGSSTPSGATGSPGTPGSGTASSSPGSSTPSGATGSPGTPGSGTASSSPGSSTPSGATGSPGSSTPS |
| | GATGSPGSSPSASTGTGPGSSPSASTGTGPGASPGTSSTGSPGTPGSGTASSSPGSSTPSGATGSPG |
| | SSPSASTGTGPGSSPSASTGTGPGASPGTSSTGSPGASPGTSSTGSPGSSTPSGATGSPGSSPSAST |
| | GTGPGASPGTSSTGSPGSSPSASTGTGPGTPGSGTASSSPGSSTP |
| AG684A | TSSTGSPGTPGSGTASSSPGSSTPSGATGSPGSSPSASTGTGPGSSPSASTGTGPGSSTPSGATGSP |
| | GSSTPSGATGSPGASPGTSSTGSPGASPGTSSTGSPGASPGTSSTGSPGTPGSGTASSSPGASPGTS |
| | STGSPGASPGTSSTGSPGASPGTSSTGSPGSSPSASTGTGPGTPGSGTASSSPGASPGTSSTGSPGA |
| | SPGTSSTGSPGASPGTSSTGSPGSSTPSGATGSPGSSTPSGATGSPGASPGTSSTGSPGTPGSGTAS |
| | SSPGSSTPSGATGSPGSSTPSGATGSPGSSTPSGATGSPGSSPSASTGTGPGASPGTSSTGSPGASP |
| | GTSSTGSPGTPGSGTASSSPGASPGTSSTGSPGASPGTSSTGSPGASPGTSSTGSPGASPGTSSTGS |
| | PGTPGSGTASSSPGSSTPSGATGSPGTPGSGTASSSPGSSTPSGATGSPGTPGSGTASSSPGSSTPS |
| | GATGSPGSSTPSGATGSPGSSPSASTGTGPGSSPSASTGTGPGASPGTSSTGSPGTPGSGTASSSPG |
| | SSTPSGATGSPGSSPSASTGTGPGSSPSASTGTGPGASPGTSSTGSPGASPGTSSTGSPGSSTPSGA |
| | TGSPGSSPSASTGTGPGASPGTSSTGSPGSSPSASTGTGPGTPGSGTASSSPGSSTPSGATGSPGSS |
| | TPSGATGSPGASPG |
| AG720A | TSSTGSPGTPGSGTASSSPGSSTPSGATGSPGASPGTSSTGSPGTPGSGTASSSPGSSTPSGATGSP |
| | GSSPSASTGTGPGSSPSASTGTGPGSSTPSGATGSPGSSTPSGATGSPGASPGTSSTGSPGASPGTS |
| | STGSPGASPGTSSTGSPGTPGSGTASSSPGASPGTSSTGSPGASPGTSSTGSPGASPGTSSTGSPGS |
| | SPSASTGTGPGTPGSGTASSSPGASPGTSSTGSPGASPGTSSTGSPGASPGTSSTGSPGSSTPSGAT |
| | GSPGSSTPSGATGSPGASPGTSSTGSPGTPGSGTASSSPGSSTPSGATGSPGSSTPSGATGSPGSST |
| | PSGATGSPGSSPSASTGTGPGASPGTSSTGSPGASPGTSSTGSPGTPGSGTASSSPGASPGTSSTGS |
| | PGASPGTSSTGSPGASPGTSSTGSPGASPGTSSTGSPGTPGSGTASSSPGSSTPSGATGSPGTPGSG |
| | TASSSPGSSTPSGATGSPGTPGSGTASSSPGSSTPSGATGSPGSSTPSGATGSPGSSPSASTGTGPG |
| | SSPSASTGTGPGASPGTSSTGSPGTPGSGTASSSPGSSTPSGATGSPGSSPSASTGTGPGSSPSAST |
| | GTGPGASPGTSSTGSPGASPGTSSTGSPGSSTPSGATGSPGSSPSASTGTGPGASPGTSSTGSPGSS |
| | PSASTGTGPGTPGSGTASSSPGSSTPSGATGSPGSSTPSGATGSPGASPG |
| AG756A | TSSTGSPGSSPSASTGTGPGSSPSASTGTGPGTPGSGTASSSPGSSTPSGATGSPGSSPSASTGTGP |
| | GASPGTSSTGSPGTPGSGTASSSPGSSTPSGATGSPGTPGSGTASSSPGASPGTSSTGSPGASPGTS |
| | STGSPGTPGSGTASSSPGSSTPSGATGSPGASPGTSSTGSPGTPGSGTASSSPGSSTPSGATGSPGS |
| | SPSASTGTGPGSSPSASTGTGPGSSTPSGATGSPGSSTPSGATGSPGASPGTSSTGSPGASPGTSST |
| | GSPGASPGTSSTGSPGTPGSGTASSSPGASPGTSSTGSPGASPGTSSTGSPGASPGTSSTGSPGSSPS |
| | ASTGTGPGTPGSGTASSSPGASPGTSSTGSPGASPGTSSTGSPGASPGTSSTGSPGSSTPSGATGSP |
| | GSSTPSGATGSPGASPGTSSTGSPGTPGSGTASSSPGSSTPSGATGSPGSSTPSGATGSPGSSTPSG |
| | ATGSPGSSPSASTGTGPGASPGTSSTGSPGASPGTSSTGSPGTPGSGTASSSPGASPGTSSTGSPGA |
| | SPGTSSTGSPGASPGTSSTGSPGASPGTSSTGSPGTPGSGTASSSPGSSTPSGATGSPGTPGSGTAS |
| | SSPGSSTPSGATGSPGTPGSGTASSSPGSSTPSGATGSPGSSTPSGATGSPGSSPSASTGTGPGSSPS |
| | ASTGTGPGASPGTSSTGSPGTPGSGTASSSPGSSTPSGATGSPGSSPSASTGTGPGSSPSASTGTGP |
| | GASPGTSSTGSPGASPG |
| AG792A | TSSTGSPGSSPSASTGTGPGSSPSASTGTGPGTPGSGTASSSPGSSTPSGATGSPGSSPSASTGTGP |
| | GASPGTSSTGSPGTPGSGTASSSPGSSTPSGATGSPGTPGSGTASSSPGASPGTSSTGSPGASPGTS |
| | STGSPGTPGSGTASSSPGSSTPSGATGSPGASPGTSSTGSPGTPGSGTASSSPGSSTPSGATGSPGS |
| | SPSASTGTGPGSSPSASTGTGPGSSTPSGATGSPGSSTPSGATGSPGASPGTSSTGSPGASPGTSST |
| | GSPGASPGTSSTGSPGTPGSGTASSSPGASPGTSSTGSPGASPGTSSTGSPGASPGTSSTGSPGSSPS |
| | ASTGTGPGTPGSGTASSSPGASPGTSSTGSPGASPGTSSTGSPGASPGTSSTGSPGSSTPSGATGSP |
| | GSSTPSGATGSPGASPGTSSTGSPGTPGSGTASSSPGSSTPSGATGSPGSSTPSGATGSPGSSTPSG |
| | ATGSPGSSPSASTGTGPGASPGTSSTGSPGASPGTSSTGSPGTPGSGTASSSPGASPGTSSTGSPGA |
| | SPGTSSTGSPGASPGTSSTGSPGASPGTSSTGSPGTPGSGTASSSPGSSTPSGATGSPGTPGSGTAS |
| | SSPGSSTPSGATGSPGTPGSGTASSSPGSSTPSGATGSPGSSTPSGATGSPGSSPSASTGTGPGSSPS |
| | ASTGTGPGASPGTSSTGSPGTPGSGTASSSPGSSTPSGATGSPGSSPSASTGTGPGSSPSASTGTGP |
| | GASPGTSSTGSPGASPGTSSTGSPGSSTPSGATGSPGSSPSASTGTGPGASPG |
| AG828A | TSSTGSPGSSPSASTGTGPGSSPSASTGTGPGTPGSGTASSSPGSSTPSGATGSPGSSPSASTGTGP |
| | GASPGTSSTGSPGTPGSGTASSSPGSSTPSGATGSPGTPGSGTASSSPGASPGTSSTGSPGASPGTS |
| | STGSPGTPGSGTASSSPGSSTPSGATGSPGASPGTSSTGSPGTPGSGTASSSPGSSTPSGATGSPGS |
| | SPSASTGTGPGSSPSASTGTGPGSSTPSGATGSPGSSTPSGATGSPGASPGTSSTGSPGASPGTSST |
| | GSPGASPGTSSTGSPGTPGSGTASSSPGASPGTSSTGSPGASPGTSSTGSPGASPGTSSTGSPGSSPS |
| | ASTGTGPGTPGSGTASSSPGASPGTSSTGSPGASPGTSSTGSPGASPGTSSTGSPGSSTPSGATGSP |
| | GSSTPSGATGSPGASPGTSSTGSPGTPGSGTASSSPGSSTPSGATGSPGSSTPSGATGSPGSSTPSG |
| | ATGSPGSSPSASTGTGPGASPGTSSTGSPGASPGTSSTGSPGTPGSGTASSSPGASPGTSSTGSPGA |
| | SPGTSSTGSPGASPGTSSTGSPGASPGTSSTGSPGTPGSGTASSSPGSSTPSGATGSPGTPGSGTAS |
| | SSPGSSTPSGATGSPGTPGSGTASSSPGSSTPSGATGSPGSSTPSGATGSPGSSPSASTGTGPGSSPS |
| | ASTGTGPGASPGTSSTGSPGTPGSGTASSSPGSSTPSGATGSPGSSPSASTGTGPGSSPSASTGTGP |
| | GASPGTSSTGSPGASPGTSSTGSPGSSTPSGATGSPGSSPSASTGTGPGASPGTSSTGSPGSSPSAS |
| | TGTGPGTPGSGTASSSPGSSTP |

In other embodiments, the CFXTEN composition comprises one or more non-repetitive XTEN sequences of about 36 to about 3000 amino acid residues, wherein at least about 80%, or at least about 90%, or about 91%, or about 92%, or about 93%, or about 94%, or about 95%, or about 96%, or about 97%, or about 98%, or about 99% to about 100% of the sequence consists of non-overlapping 36 amino acid sequence motifs selected from one or more of the polypeptide sequences of Tables 9-12, either as a family sequence, or where motifs are selected from two or more families of motifs.

In those embodiments wherein the XTEN component of the CFXTEN fusion protein has less than 100% of its amino acids consisting of 4, 5, or 6 types of amino acid selected from glycine (G), alanine (A), serine (S), threonine (T), glutamate (E) and proline (P), or less than 100% of the sequence consisting of the sequence motifs from Table 3 or the XTEN sequences of Tables 4, and 9-13 or less than 100% sequence identity compared with an XTEN from Tables 4, and 9-13, the other amino acid residues of the XTEN are selected from any of the other 14 natural L-amino acids, but are preferentially selected from hydrophilic amino acids such that the XTEN sequence contains at least about 90%, or at least about 91%, or at least about 92%, or at least about 93%, or at least about 94%, or at least about 95%, or at least about 96%, or at least about 97%, or at least about 98%, or at least about 99% hydrophilic amino acids. The XTEN amino acids that are not glycine (G), alanine (A), serine (S), threonine (T), glutamate (E) and proline (P) are either interspersed throughout the XTEN sequence, are located within or between the sequence motifs, or are concentrated in one or more short stretches of the XTEN sequence, e.g., to create a linker to the FVIII component. In such cases where the XTEN component of the CFXTEN comprises amino acids other than glycine (G), alanine (A), serine (S), threonine (T), glutamate (E) and proline (P), it is preferred that less than about 2% or less than about 1% of the amino acids be hydrophobic residues Without wishing to be bound by one particular theory, the resulting sequences generally lack a secondary structure, e.g., not having more than 2% alpha helices or 2% beta-sheets, as determined by the methods disclosed herein. Hydrophobic residues that are less favored in construction of XTEN include tryptophan, phenylalanine, tyrosine, leucine, isoleucine, valine, and methionine. Additionally, one can design the XTEN sequences to contain less than 5% or less than 4% or less than 3% or less than 2% or less than 1% or none of the following amino acids: cysteine (to avoid disulfide formation and oxidation), methionine (to avoid oxidation), asparagine and glutamine (to avoid desamidation). Thus, in some embodiments, the XTEN component of the CFXTEN fusion protein comprising other amino acids in addition to glycine (G), alanine (A), serine (S), threonine (T), glutamate (E) and proline (P) would have a sequence with less than 5% of the residues contributing to alpha-helices and beta-sheets as measured by the Chou-Fasman algorithm and have at least 90%, or at least about 95% or more random coil formation as measured by the GOR algorithm.

### 3. Length of Sequence

In another aspect, the invention provides XTEN of varying lengths for incorporation into CFXTEN compositions wherein the length of the XTEN sequence(s) are chosen based on the property or function to be achieved in the fusion protein. Depending on the intended property or function, the CFXTEN compositions comprise short or intermediate length XTEN located internal to the FVIII sequence or between FVIII domains and/or longer XTEN sequences that can serve as carriers, located in the fusion proteins as described herein. While not intended to be limiting, the XTEN or fragments of XTEN include short segments of about 6 to about 99 amino acid residues, intermediate lengths of about 100 to about 399 amino acid residues, and longer lengths of about 400 to about 3000 amino acid residues. Thus, the XTEN for incorporation into the subject CFXTEN encompass XTEN or fragments of XTEN with lengths of about 6, or about 12, or about 36, or about 40, or about 42, or about 72 or about 96, or about 144, or about 288, or about 400, or about 500, or about 576, or about 600, or about 700, or about 800, or about 864, or about 900, or about 1000, or about 1500, or about 2000, or about 2500, or up to about 3000 amino acid residues in length. Alternatively, the XTEN sequences can be about 6 to about 50, about 50 to about 100, about 100 to 150, about 150 to 250, about 250 to 400, about 400 to about 500, about 500 to about 900, about 900 to 1500, about 1500 to 2000, or about 2000 to about 3000 amino acid residues in length. The precise length of an XTEN can vary without adversely affecting the biological activity of a CFXTEN composition. In one embodiment, one or more of the XTEN used herein has 36 amino acids, 42 amino acids, 144 amino acids, 288 amino acids, 576 amino acids, or 864 amino acids in length. In another embodiment, one or more of the XTEN used herein is selected from the group consisting of XTEN_AE864, XTEN_AE576, XTEN_AE288, XTEN_AE144, XTEN_AE42, XTEN_AG864, XTEN_AG576, XTEN_AG288, XTEN_AG144, and XTEN_AG42. Non-limiting examples of XTEN sequences are presented in Table 4. In some embodiments, one or more of the XTEN used herein is selected from any one of the sequences in Table 4.

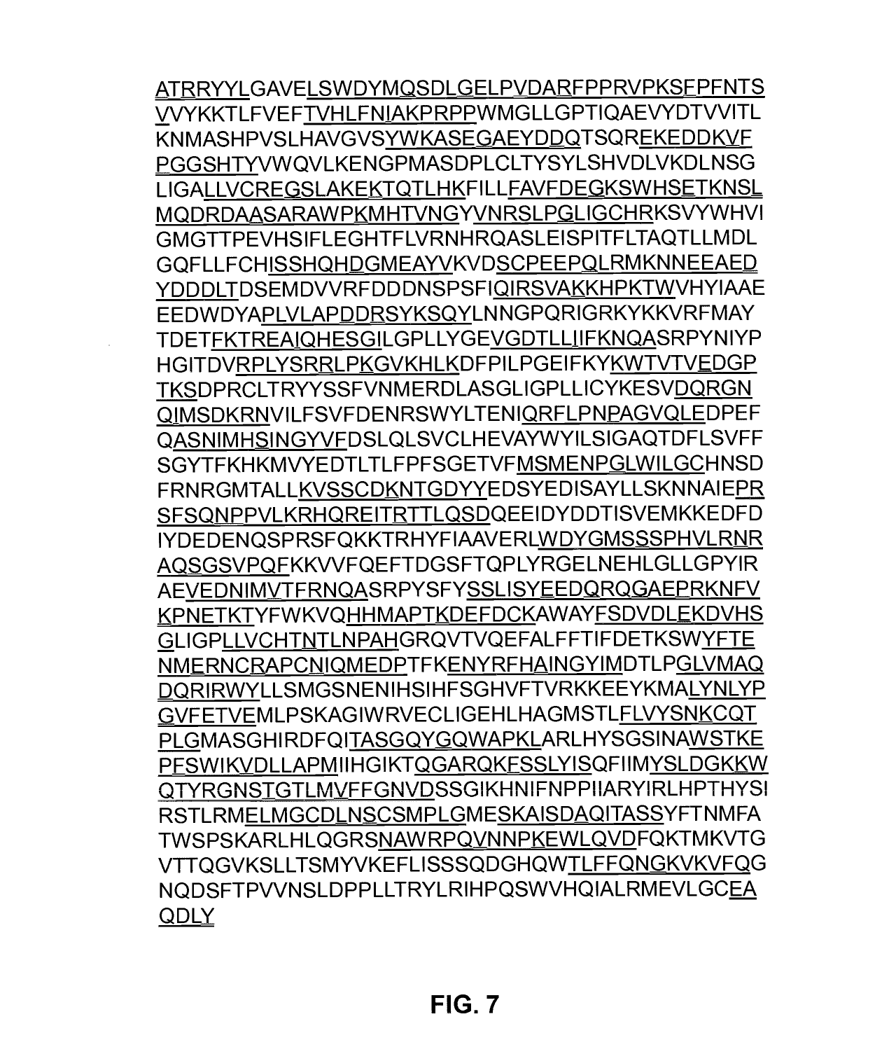

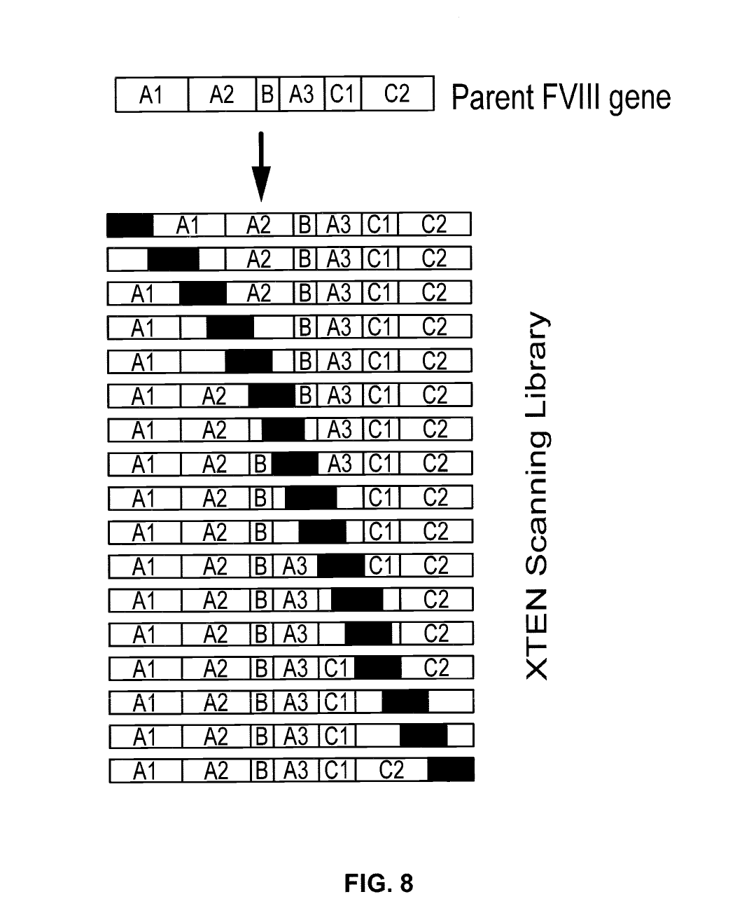

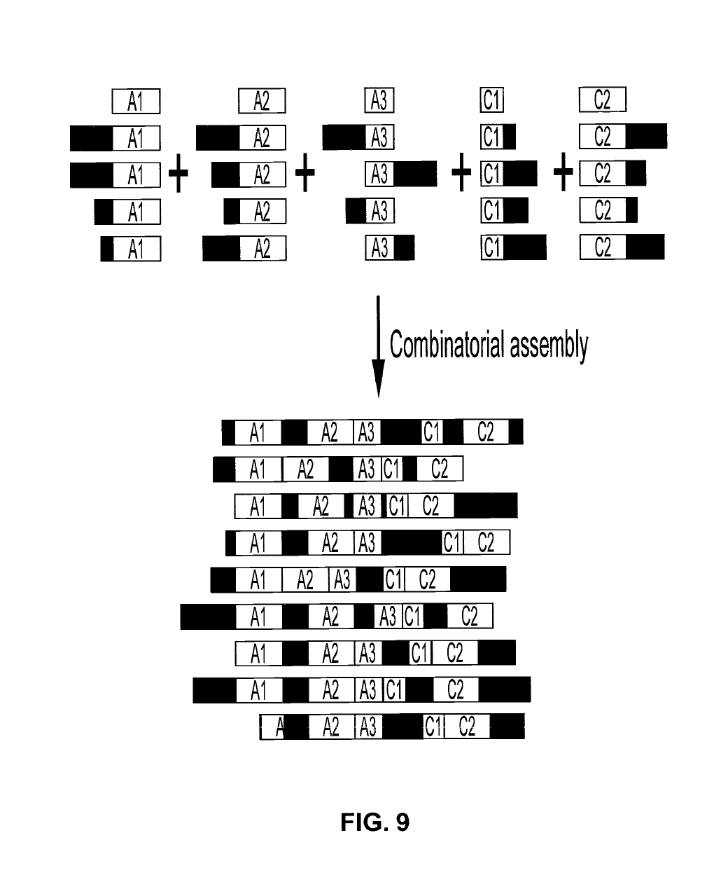

In particular CFXTEN configuration designs, where the XTEN serve as a flexible linker, or are inserted in external loops or unordered regions of the FVIII sequence to increase the bulk or hydrophilicity of the region, or are designed to interfere with clearance receptors for FVIII to enhance pharmacokinetic properties, or where a short or intermediate length of XTEN is used to facilitate tissue penetration or to vary the strength of interactions of the CFXTEN fusion protein with its target, or where it is desirable to distribute the cumulative length of XTEN in segments of short or intermediate length at multiple locations within the FVIII sequence, the invention contemplates CFXTEN compositions with one or more short or intermediate XTEN sequences inserted between one or more FVIII domains or within external loops, or at other sites in the FVIII sequence such as, but not limited to, locations at or proximal to the insertion sites identified in Table 5 or Table 25 or as illustrated in FIG. 7. In one embodiment of the foregoing, the CFXTEN fusion protein contains multiple XTEN segments, e.g., at least two, or at least three, or at least four, or at least five, or at least six or more XTEN segments in which the XTEN segments can be identical or they can be different. In other particular CFXTEN configuration designs, where the XTEN serves as a carrier to increase the bulk of the fusion protein, or to vary the strength of interactions of the CFXTEN fusion protein with its target, or to enhance the pharmacokinetic properties of the fusion protein, the invention contemplates CFXTEN compositions with one or more intermediate or longer length XTEN sequences inserted at the N- or C-termini, between one or more FVIII domains or within external loops, or at other sites in the FVIII sequence such as, but not limited to, locations at or proximal to the insertion sites identified in Table 5 or Table 25 or as illustrated in FIG. 7. The incorporation of longer XTEN into CFXTEN compositions confers enhanced properties on the fusion proteins, compared to fusion proteins with the same number of shorter length XTEN, including slower rates of systemic absorption and increased bioavailability after subcutaneous or intramuscular administration to a subject, and increased terminal half-life after parenteral administration. In the embodiments wherein the CFXTEN fusion proteins comprise multiple XTEN sequences, the cumulative length of the total residues in the XTEN sequences is greater than about 100 to about 1000, or about 200 to about 2000, or about 400 to about 3000 amino acid residues and the XTEN can be identical or they can be different in sequence, net charge, or in length. In one embodiment of CFXTEN comprising multiple XTEN, the individual XTEN sequences each exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity compared to a motif or an XTEN selected from Tables 3, 4, and 9-13 or a fragment thereof, when optimally aligned with a sequence of comparable length.

As described more fully below, methods are disclosed in which the CFXTEN are designed by selecting the length of the XTEN and its site of incorporation within the CFXTEN to confer a target half-life or other physicochemical property of a CFXTEN fusion protein, and then are incorporated into the FVIII to create the CFXTEN fusion protein compositions. In general, XTEN cumulative lengths longer that about 400 residues incorporated into the CFXTEN compositions result in longer half-life compared to shorter cumulative lengths, e.g., shorter than about 280 residues. In one embodiment, CFXTEN fusion proteins designs are contemplated that comprise a single XTEN as a carrier, with a long sequence length of at least about 400, or at least about 600, or at least about 800, or at least about 900, or at least about 1000 or more amino acids. In another embodiment, multiple XTEN are incorporated into the fusion protein to achieve cumulative lengths of at least about 400, or at least about 600, or at least about 800, or at least about 900, or at least about 1000 or more amino acids, wherein the XTEN can be identical or they can be different in sequence or length. As used herein, “cumulative length” is intended to encompass the total length, in amino acid residues, when more than one XTEN is incorporated into the CFXTEN fusion protein. Both of the foregoing embodiments are designed to confer increased bioavailability and/or increased terminal half-life after administration to a subject compared to CFXTEN comprising shorter cumulative XTEN lengths. When administered subcutaneously or intramuscularly, the Cₘₐₓ is reduced but the area under the curve (AUC) is increased in comparison to a comparable dose of a CFXTEN with shorter cumulative length XTEN or FVIII not linked to XTEN, thereby contributing to the ability to maintain effective levels of the CFXTEN composition for a longer period of time and permitting increased periods between dosing, as described more fully below. Thus, the XTEN confers the property of a depot to the administered CFXTEN, in addition to the other physicochemical properties described herein.

When XTEN are used as a carrier, the invention takes advantage of the discovery that increasing the length of the non-repetitive, unstructured polypeptides enhances the unstructured nature of the XTENs and correspondingly enhances the physical/chemical and pharmacokinetic properties of fusion proteins comprising the XTEN carrier. As described more fully in the Examples, proportional increases in the length of the XTEN, even if created by a repeated order of single family sequence motifs (e.g., the four AE motifs of Table 3), result in a sequence with a higher percentage of random coil formation, as determined by GOR algorithm, or reduced content of alpha-helices or beta-sheets, as determined by Chou-Fasman algorithm, compared to shorter XTEN lengths. In addition, increasing the length of the unstructured polypeptide fusion partner, as described in the Examples, results in a fusion protein with a disproportionate increase in terminal half-life compared to fusion proteins with unstructured polypeptide partners with shorter sequence lengths. The enhanced pharmacokinetic properties of the CFXTEN in comparison to FVIII not linked to XTEN are described more fully, below.

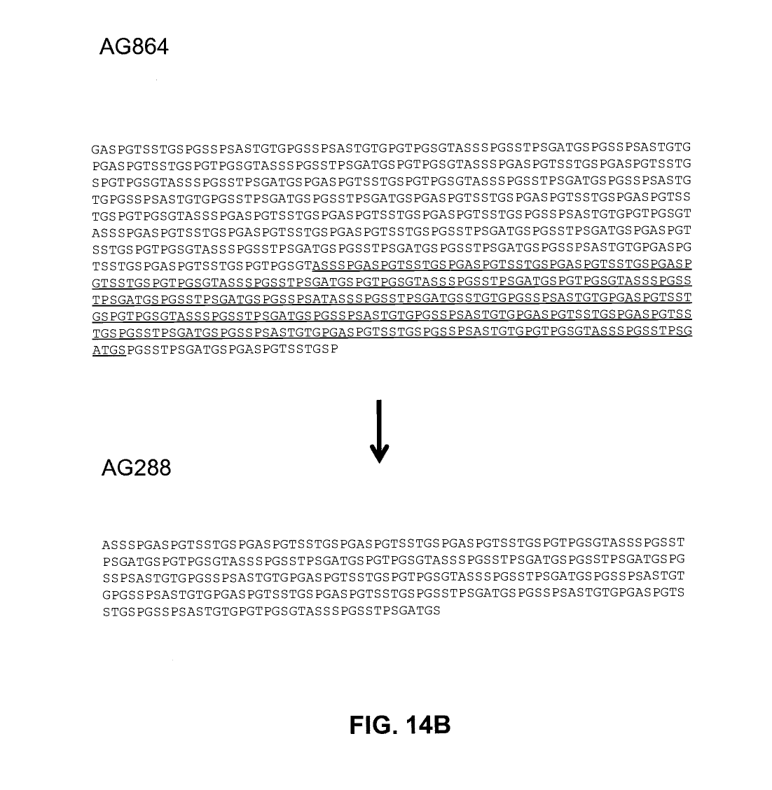

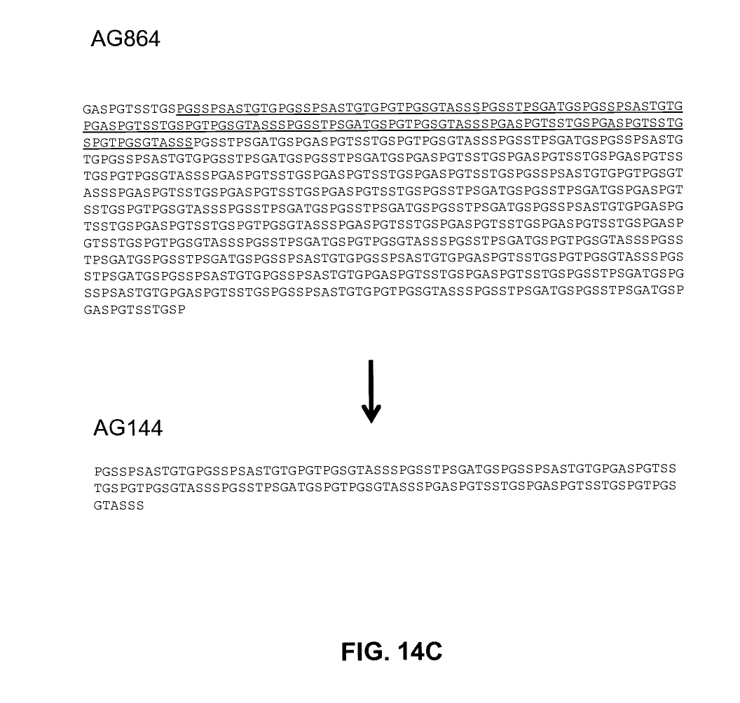

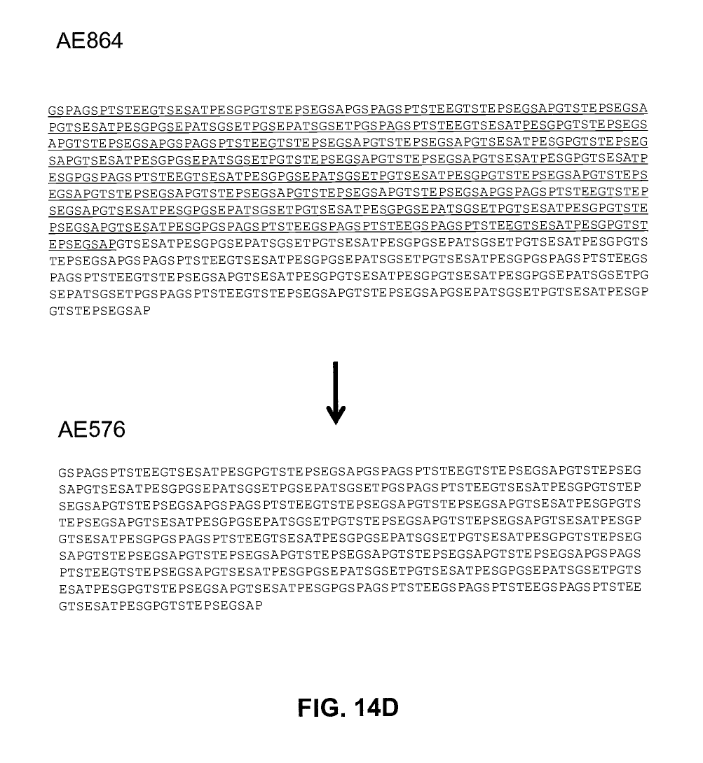

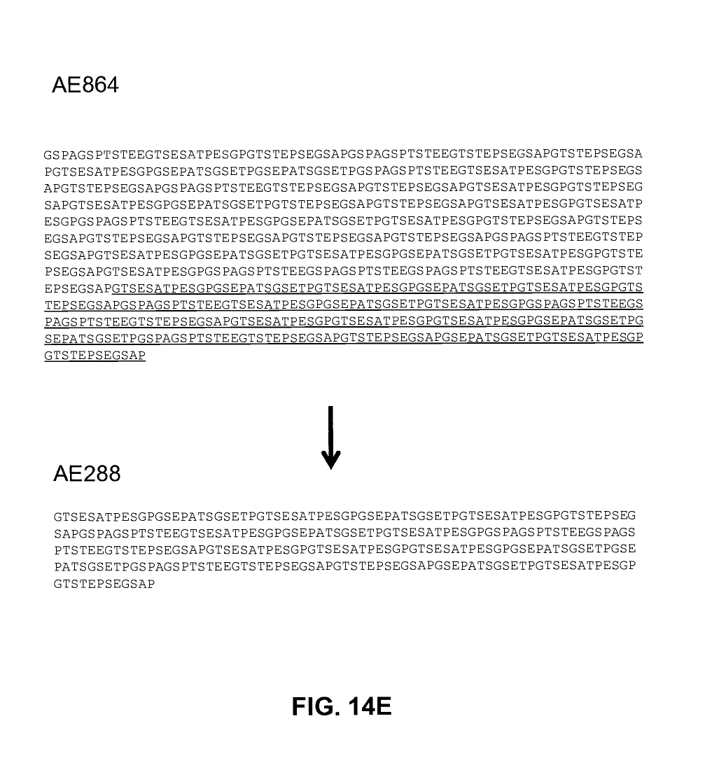

In another aspect, the invention provides methods to create XTEN of short or intermediate lengths from longer “donor” XTEN sequences, wherein the longer donor sequence is created by truncating at the N-terminus, or the C-terminus, or a fragment is created from the interior of a donor sequence, thereby resulting in a short or intermediate length XTEN. In non-limiting examples, as schematically depicted in FIG. 14A-C, the AG864 sequence of 864 amino acid residues can be truncated to yield an AG 144 with 144 residues, an AG288 with 288 residues, an AG576 with 576 residues, or other intermediate lengths, while the AE864 sequence (as depicted in FIG. 14D, E) can be truncated to yield an AE288 or AE576 or other intermediate lengths. It is specifically contemplated that such an approach can be utilized with any of the XTEN embodiments described herein or with any of the sequences listed in Tables 4 or 9-13 to result in XTEN of a desired length.

### 4. Net Charge

In other embodiments, the unstructured characteristic of an XTEN polypeptide can be enhanced by incorporation of amino acid residues with a net charge and/or reduction of the overall percentage (e.g. less than 5%, or 4%, or 3%, or 2%, or 1%) of hydrophobic amino acids in the XTEN sequence. The overall net charge and net charge density is controlled by modifying the content of charged amino acids in the XTEN sequences, either positive or negative, with the net charge typically represented as the percentage of amino acids in the polypeptide contributing to a charged state beyond those residues that are cancelled by a residue with an opposite charge. In some embodiments, the net charge density of the XTEN of the compositions may be above +0.1 or below −0.1 charges/residue. By “net charge density” of a protein or peptide herein is meant the net charge divided by the total number of amino acids in the protein or propeptide. In other embodiments, the net charge of an XTEN can be about 0%, about 1%, about 2%, about 3%, about 4%, about 5%, about 6%, about 7%, about 8%, about 9%, about 10% about 11%, about 12%, about 13%, about 14%, about 15%, about 16%, about 17%, about 18%, about 19%, or about 20% or more. Based on the net charge, some XTENs have an isoelectric point (pI) of 1.0, 1.5, 2.0, 2.5, 3.0, 3.5, 4.0, 4.5, 5.0, 5.5, 6.0, or even 6.5. In preferred embodiments, the XTEN will have an isoelectric point between 1.5 and 4.5 and carry a net negative charge under physiologic conditions.

Since most tissues and surfaces in a human or animal have a net negative charge, in some embodiments the XTEN sequences are designed to have a net negative charge to minimize non-specific interactions between the XTEN containing compositions and various surfaces such as blood vessels, healthy tissues, or various receptors. Not to be bound by a particular theory, an XTEN can adopt open conformations due to electrostatic repulsion between individual amino acids of the XTEN polypeptide that individually carry a net negative charge and that are distributed across the sequence of the XTEN polypeptide. In some embodiments, the XTEN sequence is designed with at least 90% or 95% of the charged residues separated by other residues such as serine, alanine, threonine, proline or glycine, which leads to a more uniform distribution of charge, better expression or purification behavior. Such a distribution of net negative charge in the extended sequence lengths of XTEN can lead to an unstructured conformation that, in turn, can result in an effective increase in hydrodynamic radius. In preferred embodiments, the negative charge of the subject XTEN is conferred by incorporation of glutamic acid residues. Generally, the glutamic residues are spaced uniformly across the XTEN sequence. In some cases, the XTEN can contain about 10-80, or about 15-60, or about 20-50 glutamic residues per 20 kDa of XTEN that can result in an XTEN with charged residues that would have very similar pKa, which can increase the charge homogeneity of the product and sharpen its isoelectric point, enhance the physicochemical properties of the resulting CFXTEN fusion protein for, and hence, simplifying purification procedures. For example, where an XTEN with a negative charge is desired, the XTEN can be selected solely from an AE family sequence, which has approximately a 17% net charge due to incorporated glutamic acid, or can include varying proportions of glutamic acid-containing motifs of Table 3 to provide the desired degree of net charge. Non-limiting examples of AE XTEN include, but are not limited to the AE36, AE42, AE48, AE144, AE288, AE576, AE624, AE864, and AE912 polypeptide sequences of Tables 4 and 10 or fragments thereof. In one embodiment, an XTEN sequence of Tables 4, or 9-12 can be modified to include additional glutamic acid residues to achieve the desired net negative charge. Accordingly, in one embodiment the invention provides XTEN in which the XTEN sequences contain about 1%, 2%, 4%, 8%, 10%, 15%, 17%, 20%, 25%, or even about 30% glutamic acid. In one embodiment, the invention contemplates incorporation of up to 5% aspartic acid residues into XTEN in addition to glutamic acid in order to achieve a net negative charge.

In other embodiments, where no net charge is desired, the XTEN can be selected from, for example, AG XTEN components, such as the AG motifs of Table 3, or those AM motifs of Table 3 that have no net charge. Non-limiting examples of AG XTEN include, but are not limited to AG42, AG 144, AG288, AG576, and AG864 polypeptide sequences of Tables 4 and 12, or fragments thereof. In another embodiment, the XTEN can comprise varying proportions of AE and AG motifs (in order to have a net charge that is deemed optimal for a given use or to maintain a given physicochemical property.

Not to be bound by a particular theory, the XTEN of the CFXTEN compositions with the higher net charge are expected to have less non-specific interactions with various negatively-charged surfaces such as blood vessels, tissues, or various receptors, which would further contribute to reduced active clearance. Conversely, it is believed that the XTEN of the CFXTEN compositions with a low (or no) net charge would have a higher degree of interaction with surfaces that can potentiate the activity of the associated coagulation factor, given the known contribution of cell (e.g., platelets) and vascular surfaces to the coagulation process and the intensity of activation of coagulation factors (Zhou, R., et al., Biomaterials (2005) 26(16):2965-2973; London, F., et al. Biochemistry (2000) 39(32):9850-9858).

The XTEN of the compositions of the present invention generally have no or a low content of positively charged amino acids. In some embodiments, the XTEN may have less than about 10% amino acid residues with a positive charge, or less than about 7%, or less than about 5%, or less than about 2%, or less than about 1% amino acid residues with a positive charge. However, the invention contemplates constructs where a limited number of amino acids with a positive charge, such as lysine, are incorporated into XTEN to permit conjugation between the epsilon amine of the lysine and a reactive group on a peptide, a linker bridge, or a reactive group on a drug or small molecule to be conjugated to the XTEN backbone. In one embodiment of the foregoing, the XTEN of the subject CFXTEN has between about 1 to about 100 lysine residues, or about 1 to about 70 lysine residues, or about 1 to about 50 lysine residues, or about 1 to about 30 lysine residues, or about 1 to about 20 lysine residues, or about 1 to about 10 lysine residues, or about 1 to about 5 lysine residues, or alternatively only a single lysine residue. Using the foregoing lysine-containing XTEN, fusion proteins can be constructed that comprise XTEN, a FVIII coagulation factor, plus a chemotherapeutic agent useful in the treatment of coagulopathy diseases or disorders, wherein the maximum number of molecules of the agent incorporated into the XTEN component is determined by the numbers of lysines or other amino acids with reactive side chains (e.g., cysteine) incorporated into the XTEN.

As hydrophobic amino acids impart structure to a polypeptide, the invention provides that the content of hydrophobic amino acids in the XTEN will typically be less than 5%, or less than 2%, or less than 1% hydrophobic amino acid content. In one embodiment, the amino acid content of methionine and tryptophan in the XTEN component of a CFXTEN fusion protein is typically less than 5%, or less than 2%, and most preferably less than 1%. In another embodiment, the XTEN will have a sequence that has less than 10% amino acid residues with a positive charge, or less than about 7%, or less that about 5%, or less than about 2% amino acid residues with a positive charge, the sum of methionine and tryptophan residues will be less than 2%, and the sum of asparagine and glutamine residues will be less than 5% of the total XTEN sequence.

### 5. Low Immunogenicity

In another aspect, the XTEN sequences provided herein have a low degree of immunogenicity or are substantially non-immunogenic. Several factors can contribute to the low immunogenicity of XTEN, e.g., the non-repetitive sequence, the unstructured conformation, the high degree of solubility, the low degree or lack of self-aggregation, the low degree or lack of proteolytic sites within the sequence, and the low degree or lack of epitopes in the XTEN sequence.

Conformational epitopes are formed by regions of the protein surface that are composed of multiple discontinuous amino acid sequences of the protein antigen. The precise folding of the protein brings these sequences into a well-defined, stable spatial configurations, or epitopes, that can be recognized as “foreign” by the host humoral immune system, resulting in the production of antibodies to the protein or the activation of a cell-mediated immune response. In the latter case, the immune response to a protein in an individual is heavily influenced by T-cell epitope recognition that is a function of the peptide binding specificity of that individual's HLA-DR allotype. Engagement of a MHC Class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response.

The ability of a peptide to bind a given MHC Class II molecule for presentation on the surface of an APC (antigen presenting cell) is dependent on a number of factors; most notably its primary sequence. In one embodiment, a lower degree of immunogenicity is achieved by designing XTEN sequences that resist antigen processing in antigen presenting cells, and/or choosing sequences that do not bind MHC receptors well. The invention provides CFXTEN fusion proteins with substantially non-repetitive XTEN polypeptides designed to reduce binding with MHC II receptors, as well as avoiding formation of epitopes for T-cell receptor or antibody binding, resulting in a low degree of immunogenicity. Avoidance of immunogenicity can attribute to, at least in part, a result of the conformational flexibility of XTEN sequences; i.e., the lack of secondary structure due to the selection and order of amino acid residues. For example, of particular interest are sequences having a low tendency to adapt compactly folded conformations in aqueous solution or under physiologic conditions that could result in conformational epitopes. The administration of fusion proteins comprising XTEN, using conventional therapeutic practices and dosing, would generally not result in the formation of neutralizing antibodies to the XTEN sequence, and also reduce the immunogenicity of the FVIII fusion partner in the CFXTEN compositions.

In one embodiment, the XTEN sequences utilized in the subject fusion proteins can be substantially free of epitopes recognized by human T cells. The elimination of such epitopes for the purpose of generating less immunogenic proteins has been disclosed previously; see for example WO 98/52976, WO 02/079232, and WO 00/3317 which are incorporated by reference herein. Assays for human T cell epitopes have been described (Stickler, M., et al. (2003) J Immunol Methods, 281: 95-108). Of particular interest are peptide sequences that can be oligomerized without generating T cell epitopes or non-human sequences. This is achieved by testing direct repeats of these sequences for the presence of T-cell epitopes and for the occurrence of 6 to 15-mer and, in particular, 9-mer sequences that are not human, and then altering the design of the XTEN sequence to eliminate or disrupt the epitope sequence. In some embodiments, the XTEN sequences are substantially non-immunogenic by the restriction of the numbers of epitopes of the XTEN predicted to bind MHC receptors. With a reduction in the numbers of epitopes capable of binding to MHC receptors, there is a concomitant reduction in the potential for T cell activation as well as T cell helper function, reduced B cell activation or upregulation and reduced antibody production. The low degree of predicted T-cell epitopes can be determined by epitope prediction algorithms such as, e.g., TEPITOPE (Sturniolo, T., et al. (1999) Nat Biotechnol, 17: 555-61), as shown in Example 33. The TEPITOPE score of a given peptide frame within a protein is the log of the K_{d} (dissociation constant, affinity, off-rate) of the binding of that peptide frame to multiple of the most common human MHC alleles, as disclosed in Sturniolo, T. et al. (1999) Nature Biotechnology 17:555). The score ranges over at least 20 logs, from about 10 to about −10 (corresponding to binding constraints of 10e¹⁰ K_{d} to 10e¹⁰ K_{d}), and can be reduced by avoiding hydrophobic amino acids that serve as anchor residues during peptide display on MHC, such as M, I, L, V, F. In some embodiments, an XTEN component incorporated into a CFXTEN does not have a predicted T-cell epitope at a TEPITOPE threshold score of about −5, or −6, or −7, or −8, or −9, or at a TEPITOPE score of −10. As used herein, a score of “−9” is a more stringent TEPITOPE threshold than a score of −5.

In another embodiment, the inventive XTEN sequences, including those incorporated into the subject CFXTEN fusion proteins, are rendered substantially non-immunogenic by the restriction of known proteolytic sites from the sequence of the XTEN, reducing the processing of XTEN into small peptides that can bind to MHC II receptors. In another embodiment, the XTEN sequence is rendered substantially non-immunogenic by the use a sequence that is substantially devoid of secondary structure, conferring resistance to many proteases due to the high entropy of the structure. Accordingly, the reduced TEPITOPE score and elimination of known proteolytic sites from the XTEN render the XTEN compositions, including the XTEN of the CFXTEN fusion protein compositions, substantially unable to be bound by mammalian receptors, including those of the immune system. In one embodiment, an XTEN of a CFXTEN fusion protein can have >100 nM K_{d} binding to a mammalian receptor, or greater than 500 nM K_{d}, or greater than 1 μM K_{d} towards a mammalian cell surface or circulating polypeptide receptor.

Additionally, the non-repetitive sequence and corresponding lack of epitopes of XTEN limit the ability of B cells to bind to or be activated by XTEN. A repetitive sequence is recognized and can form multivalent contacts with even a few B cells and, as a consequence of the cross-linking of multiple T-cell independent receptors, can stimulate B cell proliferation and antibody production. In contrast, while an XTEN can make contacts with many different B cells over its extended sequence, each individual B cell may only make one or a small number of contacts with an individual XTEN due to the lack of repetitiveness of the sequence. Not being to be bound by any theory, XTENs typically have a much lower tendency to stimulate proliferation of B cells and thus an immune response. In one embodiment, the CFXTEN have reduced immunogenicity as compared to the corresponding FVIII that is not fused to an XTEN. In one embodiment, the administration of up to three parenteral doses of a CFXTEN to a mammal result in detectable anti-CFXTEN IgG at a serum dilution of 1:100 but not at a dilution of 1:1000. In another embodiment, the administration of up to three parenteral doses of a CFXTEN to a mammal result in detectable anti-FVIII IgG at a serum dilution of 1:100 but not at a dilution of 1:1000. In another embodiment, the administration of up to three parenteral doses of a CFXTEN to a mammal result in detectable anti-XTEN IgG at a serum dilution of 1:100 but not at a dilution of 1:1000. In the foregoing embodiments, the mammal can be a mouse, a rat, a rabbit, or a cynomolgus monkey.

An additional feature of XTENs with non-repetitive sequences relative to sequences with a high degree of repetitiveness is non-repetitive XTENs form weaker contacts with antibodies. Antibodies are multivalent molecules. For instance, IgGs have two identical binding sites and IgMs contain 10 identical binding sites. Thus antibodies against repetitive sequences can form multivalent contacts with such repetitive sequences with high avidity, which can affect the potency and/or elimination of such repetitive sequences. In contrast, antibodies against non-repetitive XTENs may yield monovalent interactions, resulting in less likelihood of immune clearance such that the CFXTEN compositions can remain in circulation for an increased period of time. The exemplary sequences including those listed in Tables 4, 9, 10, 11, 12, and 13, or other parts of the application embodying the aforementioned feature. Increased hydrodynamic radius

In another aspect, a subject XTEN useful as a fusion partner has a high hydrodynamic radius that confers a corresponding increased apparent molecular weight to the CFXTEN fusion protein incorporating the XTEN. As detailed in Example 27, the linking of XTEN to therapeutic protein sequences results in CFXTEN compositions that can have increased hydrodynamic radii, increased apparent molecular weight, and increased apparent molecular weight factor compared to a therapeutic protein not linked to an XTEN. For example, in therapeutic applications in which prolonged half-life is desired, compositions in which an XTEN with a high hydrodynamic radius is incorporated into a fusion protein comprising a therapeutic protein can effectively enlarge the hydrodynamic radius of the composition beyond the glomerular pore size of approximately 3-5 nm (corresponding to an apparent molecular weight of about 70 kDa) (Caliceti. 2003. Pharmacokinetic and biodistribution properties of poly(ethylene glycol)-protein conjugates. Adv Drug Deliv Rev 55:1261-1277), resulting in reduced renal clearance of circulating proteins with a corresponding increase in terminal half-life and other enhanced pharmacokinetic properties. The hydrodynamic radius of a protein is determined by its molecular weight as well as by its structure, including shape or compactness. Not to be bound by a particular theory, the XTEN can adopt open conformations due to electrostatic repulsion between individual charges of the peptide or the inherent flexibility imparted by the particular amino acids in the sequence that lack potential to confer secondary structure. The open, extended and unstructured conformation of the XTEN polypeptide can have a greater proportional hydrodynamic radius compared to polypeptides of a comparable sequence length and/or molecular weight that have secondary and/or tertiary structure, such as typical globular proteins. Methods for determining the hydrodynamic radius are well known in the art, such as by the use of size exclusion chromatography (SEC), as described in U.S. Pat. Nos. 6,406,632 and 7,294,513. Example 27 demonstrates that increases in XTEN length result in proportional increase in the hydrodynamic radius, apparent molecular weight, and/or apparent molecular weight factor, and thus permit the tailoring of CFXTEN to desired cut-off values of apparent molecular weights or hydrodynamic radii. Accordingly, in certain embodiments, the CFXTEN fusion protein can be configured with an XTEN such that the fusion protein can have a hydrodynamic radius of at least about 5 nm, or at least about 8 nm, or at least about 10 nm, or 12 nm, or at least about 15 nm. In the foregoing embodiments, the large hydrodynamic radius conferred by the XTEN in a CFXTEN fusion protein can lead to reduced renal clearance of the resulting fusion protein, leading to a corresponding increase in terminal half-life, an increase in mean residence time, and/or a decrease in renal clearance rate.

Generally, the actual molecular weight of the FVIII component of the CFXTEN fusion protein is about 165-170 kDa. In the case of a FVIII BDD, it is about 265 kDa for the mature form of full-length FVIII, while the actual molecular weight of a CFXTEN fusion protein for a FVIII BDD plus a single or multiple XTEN ranges from about 200 to about 270 kDa, depending on the length of the XTEN component. When the molecular weights of the CFXTEN fusion proteins are derived from size exclusion chromatography analyses, the open conformation of the XTEN due to the low degree of secondary structure results in an increase in the apparent molecular weight of the fusion proteins. In some embodiments, the CFXTEN comprising a FVIII and at least one or multiple XTEN exhibits an apparent molecular weight of at least about 400 kD, or at least about 500 kD, or at least about 700 kD, or at least about 1000 kD, or at least about 1400 kD, or at least about 1600 kD, or at least about 1800 kD, or at least about 2000 kD. Accordingly, the CFXTEN fusion proteins comprising one or more XTEN exhibit an apparent molecular weight that is about 1.3-fold greater, or about 2-fold greater, or about 3-fold greater or about 4-fold greater, or about 8-fold greater, or about 10-fold greater, or about 12-fold greater, or about 15-fold greater than the actual molecular weight of the fusion protein. In one embodiment, the isolated CFXTEN fusion protein of any of the embodiments disclosed herein exhibit an apparent molecular weight factor under physiologic conditions that is greater than about 1.3, or about 2, or about 3, or about 4, or about 5, or about 6, or about 7, or about 8, or about 10, or greater than about 15. In another embodiment, the CFXTEN fusion protein has, under physiologic conditions, an apparent molecular weight factor that is about 3 to about 20, or is about 5 to about 15, or is about 8 to about 12, or is about 9 to about 10 relative to the actual molecular weight of the fusion protein. It is believed that the increased apparent molecular weight of the subject CFXTEN compositions enhances the pharmacokinetic properties of the fusion proteins by a combination of factors, which include reduced glomerular filtration, reduced active clearance, and reduced loss in capillary and venous bleeding.

### IV). CFXTEN Compositions

The present invention provides compositions comprising fusion proteins having factor VIII linked to one or more XTEN sequences, wherein the fusion protein acts to replace or augment the amount of existing FVIII in the intrinsic or contact activated coagulation pathway when administered into a subject. The invention addresses a long-felt need in increasing the terminal half-life of exogenously administered factor VIII to a subject in need thereof. One way to increase the circulation half-life of a therapeutic protein is to ensure that renal clearance or metabolism of the protein is reduced. Another way to increase the terminal half-life is to reduce the active clearance of the therapeutic protein, whether mediated by receptors, active metabolism of the protein, or other endogenous mechanisms. Both may be achieved by conjugating the protein to a polymer, which, on one hand, is capable of conferring an increased molecular size (or hydrodynamic radius) to the protein and, hence, reduced renal clearance, and, on the other hand, interferes with binding of the protein to clearance receptors or other proteins that contribute to metabolism or clearance. Thus, certain objects of the present invention include, but are not limited to, providing improved FVIII molecules with a longer circulation or terminal half-life, decreasing the number or frequency of necessary administrations of FVIII compositions, retaining at least a portion of the activity compared to native coagulation factor VIII, and/or enhancing the ability to treat coagulation deficiencies and uncontrolled bleedings more efficiently, more effectively, more economically, and/or with greater safety compared to presently available factor VIII preparations.

Accordingly, the present invention provides isolated fusion protein compositions comprising an FVIII covalently linked to one or more extended recombinant polypeptides (“XTEN”), resulting in a CFXTEN fusion protein composition. The term “CFXTEN”, as used herein, is meant to encompass fusion polypeptides that comprise one or more payload regions comprising a FVIII or a portion of a FVIII that is capable of procoagulant activity associated with a FVIII coagulation factor and at least one other region comprising at least a first XTEN polypeptide. In one embodiment, the FVIII is native FVIII. In another embodiment, the FVIII is a sequence variant, fragment, homolog, or mimetic of a natural sequence that retains at least a portion of the procoagulant activity of native FVIII, as disclosed herein. Non-limiting examples of FVIII suitable for inclusion in the compositions include the sequences of Table 1 and Table 31 or sequences having at least 80%, or at least 90%, or at least 91%, or at least 92%, or at least 93%, or at least 94%, or at least 95%, or at least 96%, or at least 97%, or at least 98%, or at least 99% sequence identity to a sequence of Table 1 or Table 31. In a preferred embodiment, the FVIII is a B-domain deleted (BDD) FVIII sequence variant, such as those BDD sequences from Table 1, Table 31 or other such sequences known in the art.

The compositions of the invention include fusion proteins that are useful, when administered to a subject in need thereof, for mediating or preventing or ameliorating a disease, disorder or condition associated with factor VIII deficiencies or defects in endogenously produced FVIII, or bleeding disorders associated with trauma, surgery, factor VIII deficiencies or defects. Of particular interest are CFXTEN fusion protein compositions for which an increase in a pharmacokinetic parameter, increased solubility, increased stability, or some other enhanced pharmaceutical property compared to native FVIII is sought, or for which increasing the terminal half-life would improve efficacy, safety, or result in reduced dosing frequency and/or improve patient management. The CFXTEN fusion proteins of the embodiments disclosed herein exhibit one or more or any combination of the improved properties and/or the embodiments as detailed herein. In some embodiments, the CFXTEN fusion composition remains at a level above a threshold value of at least 0.01-0.05, or 0.05 to 0.1, or 0.1 to 0.4 IU/ml when administered to a subject, for a longer period of time when compared to a FVIII not linked to XTEN.

The FVIII of the subject compositions, particularly those disclosed in Table 1, together with their corresponding nucleic acid and amino acid sequences, are available in public databases such as Chemical Abstracts Services Databases (e.g., the CAS Registry), GenBank, The Universal Protein Resource (UniProt) and subscription provided databases such as GenSeq (e.g., Derwent). Polynucleotide sequences applicable for expressing the subject CFXTEN sequences may be a wild type polynucleotide sequence encoding a given FVIII (e.g., either full length or mature), or in some instances the sequence may be a variant of the wild type polynucleotide sequence (e.g., a polynucleotide which encodes the wild type biologically active protein, wherein the DNA sequence of the polynucleotide has been optimized, for example, for expression in a particular species, or a polynucleotide encoding a variant of the wild type protein, such as a site directed mutant or an allelic variant. It is well within the ability of the skilled artisan to use a wild-type or consensus cDNA sequence or a codon-optimized variant of a FVIII to create CFXTEN constructs contemplated by the invention using methods known in the art and/or in conjunction with the guidance and methods provided herein, and described more fully in the Examples.

In one embodiment, a CFXTEN fusion protein comprises a single FVIII molecule linked to a single XTEN (e.g., an XTEN as described above) including, but limited to sequences AE42, AG42, AE288, AG288, AE864, and AG864 shown in Table 4. In another embodiment, the CFXTEN comprises a single FVIII linked to two XTEN, wherein the XTEN may be identical or they may be different. In another embodiment, the CFXTEN fusion protein comprises a single FVIII molecule linked to one, two, three, four, five or more XTEN sequences, in which the FVIII is a sequence that has at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity compared to a protein sequence selected from Table 1, when optimally aligned, and the one or more XTEN are each having at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity compared to one or more sequences selected from any one of Tables 3, 4, and 9-13, when optimally aligned. In yet another embodiment, the CFXTEN fusion protein comprises a single FVIII that has portions of its sequence exhibiting at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity compared to sequences of comparable length selected from Table 1, when optimally aligned, with the portions interspersed with and linked by three or more XTEN sequences that each has at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity compared to sequences selected from any one of Tables 3, 4, and 9-13, or fragments thereof, when optimally aligned. In yet another embodiment, the CFXTEN fusion protein comprises a sequence with at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to a sequence from any one of Tables 14 and 28-30, when optimally aligned.

1. CFXTEN Fusion Protein Configurations

The invention provides CFXTEN fusion protein compositions with the CF and XTEN components linked in specific N— to C-terminus configurations.

In one embodiment of the CFXTEN composition, the invention provides a fusion protein of formula I:

(XTEN)ₓ-CF-(XTEN)_{y} II

wherein independently for each occurrence, CF is a factor VIII as defined herein, including sequences of Table 1 and Table 31 (e.g., native mature FVIII, FVIII BDD-2, and FVIII BDD-9); x is either 0 or 1 and y is either 0 or 1 wherein x+y≧1; and XTEN is an extended recombinant polypeptide as described herein, including, but not limited to AE42, AG42, AE288, AG288, AE864, and AG864. Accordingly, the CFXTEN fusion composition can have XTEN-CF, XTEN-CF-XTEN, or CF-XTEN configurations.

In another embodiment of the CFXTEN composition, the invention provides a fusion protein of formula II:

(XTEN)ₓ-(S)ₓ-(CF)-(XTEN)_{y} II

wherein independently for each occurrence, CF is a factor VIII as defined herein, including sequences of Table 1 and Table 31 (e.g., native mature FVIII, FVIII BDD-2, and FVIII BDD-9); S is a spacer sequence having between 1 to about 50 amino acid residues that can optionally include a cleavage sequence or amino acids compatible with restrictions sites; x is either 0 or 1 and y is either 0 or 1 wherein x+y≧1; and XTEN is an extended recombinant polypeptide as described herein including, but not limited to AE42, AG42, AE288, AG288, AE864, and AG864.

In another embodiment of the CFXTEN composition, the invention provides an isolated fusion protein, wherein the fusion protein is of formula III:

(XTEN)ₓ-(S)ₓ-(CF)-(S)_{y}-(XTEN)_{y} III

wherein independently for each occurrence, CF is a factor VIII as defined herein, including sequences of Table 1 and Table 31 (e.g., native mature FVIII, FVIII BDD-2, and FVIII BDD-9); S is a spacer sequence having between 1 to about 50 amino acid residues that can optionally include a cleavage sequence or amino acids compatible with restrictions sites; x is either 0 or 1 and y is either 0 or 1 wherein x+y≧1; and XTEN is an extended recombinant polypeptide as described herein including, but not limited to AE42, AG42, AE288, AG288, AE864, and AG864.

In another embodiment of the CFXTEN composition, the invention provides an isolated fusion protein of formula IV:

(A1)-(XTEN)ᵤ-(A2)-(XTEN)ᵥ-(B)-(XTEN)_{w}-(A3)-(XTEN)ₓ-(C1)-(XTEN)_{y}-(C2) IV

wherein independently for each occurrence, A1 is an A1 domain of FVIII; A2 is an A2 domain of FVIII; A3 is an A3 domain of FVIII; B is a B domain of FVIII which can be a fragment or a splice variant of the B domain; C1 is a C1 domain of FVIII; C2 is a C2 domain of FVIII; v is either 0 or 1; w is either 0 or 1; x is either 0 or 1; y is either 0 or 1 with the proviso that u+v+x+y≧1; and XTEN is an extended recombinant polypeptide as described herein including, but not limited to AE42, AG42, AE288, AG288, AE864, and AG864.

In another embodiment of the CFXTEN composition, the invention provides an isolated fusion protein of formula V:

(XTEN)ₜ-(S)ₐ-(A1)-(S)_{b}-(XTEN)ᵤ-(S)_{b}-(A2)-(S)_{c}-(XTEN)ᵥ-(S)_{c}-(B)-(S)_{d}-(XTEN)_{w}-(S)_{d}-(A3)-(S)ₑ-(XTEN)ₓ-(S)ₑ-(C1)-(S)_{f}-(XTEN)_{y}-(S)_{f}-(C2)-(S)_{g}-(XTEN)_{z} V

wherein independently for each occurrence, A1 is an A1 domain of FVIII; A2 is an A2 domain of FVIII; A3 is an A3 domain of FVIII; B is a B domain of FVIII which can be a fragment or a splice variant of the B domain; C1 is a C1 domain of FVIII; C2 is a C2 domain of FVIII; S is a spacer sequence having between 1 to about 50 amino acid residues that can optionally include a cleavage sequence or amino acids compatible with restrictions sites; a is either 0 or 1; b is either 0 or 1; c is either 0 or 1; d is either 0 or 1; e is either 0 or 1; f is either 0 or 1; g is either 0 or 1; t is either 0 or 1; u is either 0 or 1; v is either 0 or 1; w is 0 or 1, x is either 0 or 1; y is either 0 or 1; z is either 0 or 1 with the proviso that t+u+v+w+x+y+z≧1; and XTEN is an extended recombinant polypeptide as described herein including, but not limited to AE42, AG42, AE288, AG288, AE864, and AG864.

In another embodiment of the CFXTEN composition, the invention provides an isolated fusion protein of formula VI:

(XTEN)ᵤ-(S)ₐ-(A1)-(S)_{b}-(XTEN)ᵥ-(S)_{b}-(A2)-(S)_{c}-(XTEN)_{w}-(S)_{c}-(A3)-(S)_{d}-(XTEN)ₓ-(S)_{d}-(C1)-(S)_{c}-(XTEN)_{y}-(S)_{c}-(C2)-(S)_{f}-(XTEN)_{z} VI

wherein independently for each occurrence, A1 is an A1 domain of FVIII; A2 is an A2 domain of FVIII; A3 is an A3 domain of FVIII; C1 is a C1 domain of FVIII; C2 is a C2 domain of FVIII; S is a spacer sequence having between 1 to about 50 amino acid residues that can optionally include a cleavage sequence or amino acids compatible with restrictions sites; a is either 0 or 1; b is either 0 or 1; c is either 0 or 1; d is either 0 or 1; e is either 0 or 1; f is either 0 or 1; u is either 0 or 1; v is either 0 or 1; w is 0 or 1, x is either 0 or 1; y is either 0 or 1; z is either 0 or 1 with the proviso that u+v+w+x+y+z≧1; and XTEN is an extended recombinant polypeptide as described herein including, but not limited to AE42, AG42, AE288, AG288, AE864, and AG864.

In another embodiment of the CFXTEN composition, the invention provides an isolated fusion protein of formula VII:

(SP)-(XTEN)ₓ-(CS)ₓ-(S)ₓ-(FVIII_{—}1-743)-(S)_{y}-(XTEN)_{y}-(S)_{y}-(FVIII_{—}1638-2332)-(S)_{z}-(CS)_{z}-(XTEN)_{z} VIIa

(SP)-(XTEN)ₓ-(CS)ₓ-(S)ₓ-(FVII_{—}1-743)-(S)_{y}-(XTEN)_{y}-(S)_{y}-(FVIII_{—}1638-2332)-(S)_{z}-(CS)_{z}-(XTEN)_{z} VIIb

wherein independently for each occurrence, SP is a signal peptide, preferably with sequence MQIELSTCFFLCLLRFCFS, CS is a cleavage sequence listed in Table 7, S is a spacer sequence having between 1 to about 50 amino acid residues that can optionally include amino acids compatible with restrictions sites, “FVIII_{—}1-743” is residues 1-743 of Factor FVIII and “FVIII_{—}1638-2332” is residues 1638-2332 of FVIII, “FVIII_{—}1-743” is residues 1-743 of Factor FVIII and “FVIII_{—}1638-2332” is residues 1638-2332 of FVIII, x is either 0 or 1, y is either 0 or 1, and z is either 0 or 1, wherein x+y+z>2; and XTEN is an extended recombinant polypeptide as described herein including, but not limited to AE42, AG42, AE288, AG288, AE864, and AG864. In one embodiment of formula VII, the spacer sequence is GPEGPS. In another embodiment of formula VII, the spacer sequence is a sequence from Table 6.

In another embodiment of the CFXTEN composition, the invention provides an isolated fusion protein of formula VIII:

(XTEN)ᵤ-(S)ₐ-(A1)-(S)_{b}-(XTEN)ᵥ-(S)_{b}-(A2)-(B1)-(S)_{c}-(XTEN)_{w}-(S)_{c}-(B2)-(A3)-(S)_{d}-(XTEN)ₓ-(S)_{d}-(C1)-(S)ₑ—(XTEN)_{y}-(S)ₑ-(C2)-(S)_{f}-(XTEN)_{z} FVIII

wherein independently for each occurrence, A1 is an A1 domain of FVIII; A2 is an A2 domain of FVIII; B1 is a fragment of the B domain that can have from residues 740 to 743-750 of FVIII or alternatively from about residues 741 to about residues 743-750 of FVIII; B2 is a fragment of the B domain that can have from residues 1654-1686 to 1689 of FVIII or alternatively from about residues1638 to about residues 1648 of FVIII; A3 is an A3 domain of FVIII; C1 is a C1 domain of FVIII; C2 is a C2 domain of FVIII; S is a spacer sequence having between 1 to about 50 amino acid residues that can optionally include a cleavage sequence or amino acids compatible with restrictions sites; a is either 0 or 1; b is either 0 or 1; c is either 0 or 1; d is either 0 or 1; e is either 0 or 1; f is either 0 or 1; u is either 0 or 1; v is either 0 or 1; w is 0 or 1, x is either 0 or 1; y is either 0 or 1; z is either 0 or 1 with the proviso that u+v+w+x+y+z≧1; and XTEN is an extended recombinant polypeptide as described herein including, but not limited to AE42, AG42, AE288, AG288, AE864, and AG864. In one embodiment of formula VIII, the spacer sequence is GPEGPS. In another embodiment of formula VIII, the spacer sequence is a sequence from Table 6.

The embodiments of formulae IV-VIII encompass CFXTEN configurations wherein one or more XTEN of lengths ranging from about 6 amino acids to ≧1000 amino acids (e.g., sequences selected from any one of Tables 3, 4, and 9-13 or fragments thereof, or sequences exhibiting at least about 90-98% or more sequence identity thereto) are inserted and linked between adjoining domains of the factor VIII, or are linked to the N- or C-terminus of the FVIII. The embodiments of formulae V-VIII further provide configurations wherein the XTEN are linked to FVIII domains via spacer sequences which can optionally comprise amino acids compatible with restrictions sites or can include cleavage sequences (e.g., the sequences of Tables 6 and 7, described more fully below) such that the XTEN encoding sequence can be, in the case of a restriction site, be integrated into a CFXTEN construct and, in the case of a cleavage sequence, the XTEN can be released from the fusion protein by the action of a protease appropriate for the cleavage sequence.

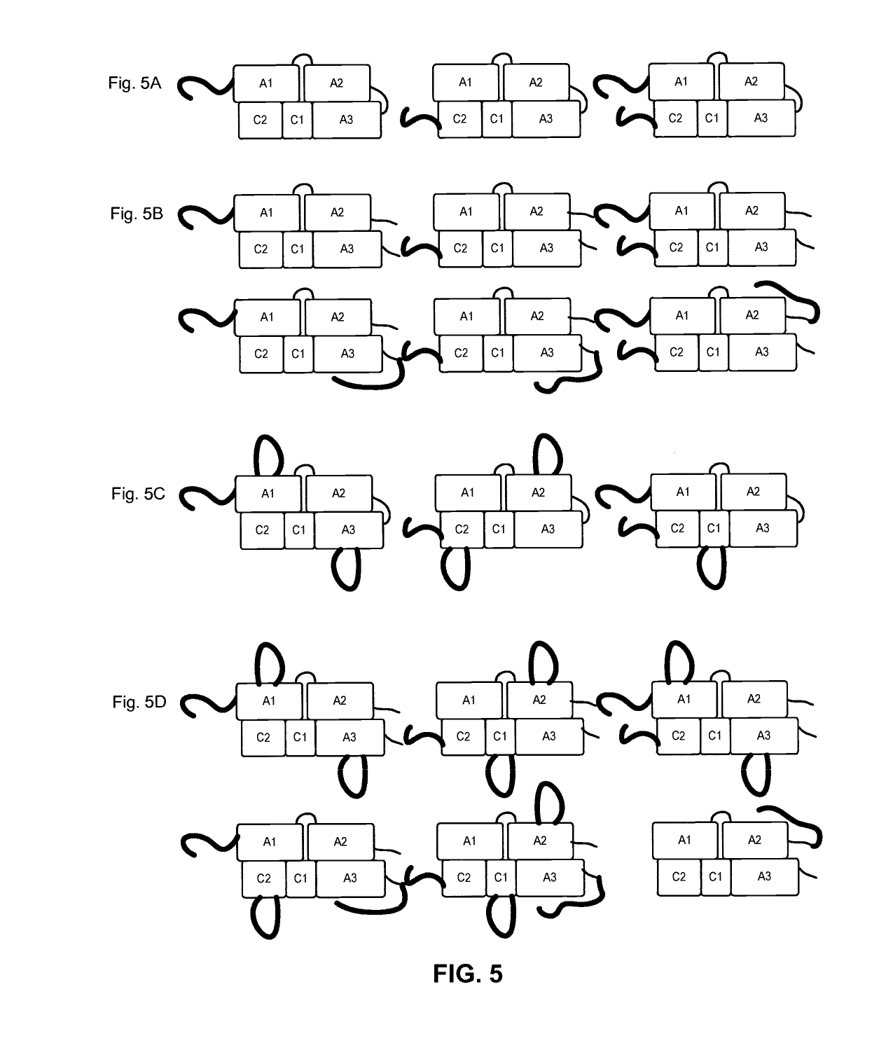

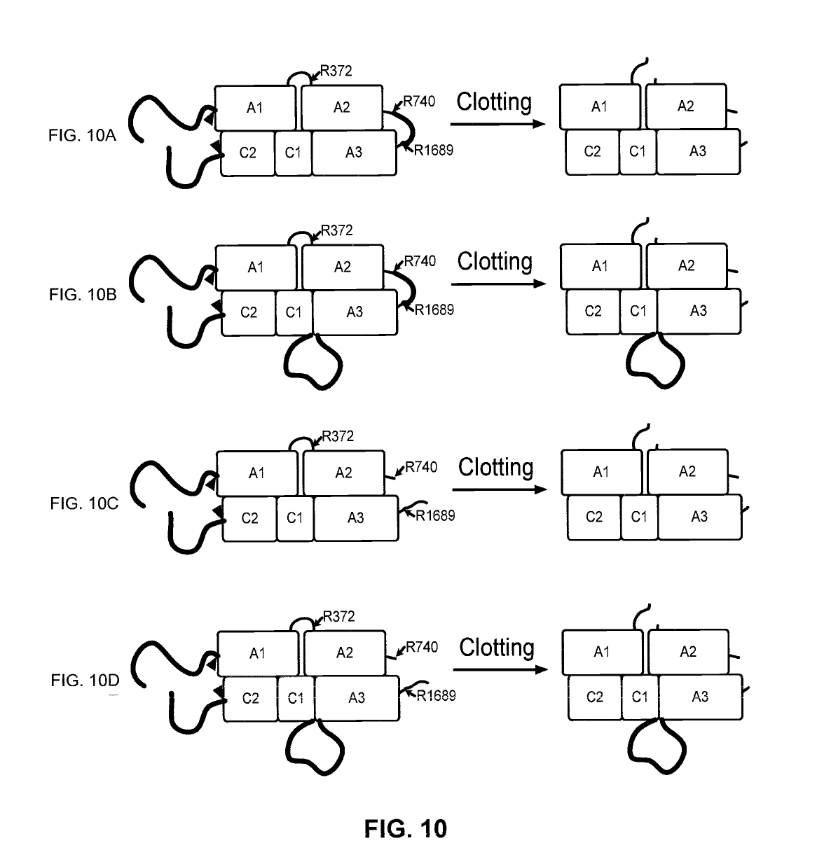

The embodiments of formulae VI-VIII differ from those of formula V in that the FVIII component of formulae VI-VIII are only the B-domain deleted forms (“FVIII BDD”) of factor VIII that retain short residual sequences of the B-domain, non-limiting examples of sequences of which are provided in Table 1, wherein one or more XTEN or fragments of XTEN of lengths ranging from about 6 amino acids to ≧1000 amino acids (e.g., sequences selected from any one of Tables 3, 4, and 9-13) are inserted and linked between adjoining domains of the factor VIII and/or between or within the remnants of the B domain residues. The invention contemplates all possible permutations of insertions of XTEN between the domains of FVIII or at or proximal to the insertion points of Table 5 or Table 25, described below, or those illustrated in FIG. 7, with optional linking of an additional XTEN to the N- or C-terminus of the FVIII, optionally linked via an additional cleavage sequence selected from Table 7, resulting in a CFXTEN composition; non-limiting examples of which are portrayed in FIGS. 5 and 10. In one embodiment, the CFXTEN comprises a FVIII BDD sequence of Table 1 or Table 31 in which one or more XTEN that each has at least about 80%, or at least about 90%, or at least about 95% or more sequence identity compared to a sequence from any one of Tables 3, 4, and 9-13 or fragments thereof are inserted between any two of the residual B domain amino acids of the FVIII BDD sequence, resulting in a single chain FVIII fusion protein, wherein the CFXTEN retains at least about 30%, or at least about 40%, or at least about 50%, or at least about 60%, or at least about 70%, or at least about 80%, or at least about 90% of the procoagulant activity of native FVIII. In the foregoing embodiment, the CFXTEN can have an additional XTEN sequence of any one of Tables 4, and 9-13 linked to the N- or C-terminus of the fusion protein. In one embodiment of a fusion protein of formula VII, the CFXTEN comprises a FVIII BDD sequence of Table 1 or Table 31 in which two or more XTEN that each has at least about 80%, or at least about 90%, or at least about 95%, or at least about 96%, or at least about 97%, or at least about 98%, or at least about 99%, or 100% sequence identity compared to a sequence from any one of Tables 3, 4, and 9-13 or fragments thereof are linked to a FVIII-BDD sequence in which at least one XTEN is inserted from about 3 to about 20 amino acid residues to the C-terminus side of the FVIII cleavage site amino acid R740 and from about 3 to about 20 amino acid residues to the N-terminus side of the FVIII cleavage site amino acid R1689 of the residual B domain amino acids of the FVIII BDD sequence, resulting in a single chain FVIII fusion protein, and one or two XTEN are linked by a cleavage sequence to the N- and/or C-terminus of the FVIII-BDD sequence, wherein the CFXTEN exhibits at least about 40%, or at least about 50%, or at least about 60%, or at least about 70%, or at least about 80%, or at least about 90% of the procoagulant activity of native FVIII after release of the XTEN by cleavage of the cleavage sequences.

In certain embodiments,

(XTEN)ᵥ-(S)ₐ-(A1)-(S)_{b}-(XTEN)_{w}-(S)_{b}-(A2)-(S)_{c}-(XTEN)ₓ-(S)_{c}-(A3)-(S)_{d}-(XTEN)_{y}-(S)_{d}-(C1)-(S)ₑ-(XTEN)_{z} (A)

wherein independently for each occurrence, A1 is an A1 domain of FVIII; A2 is an A2 domain of FVIII; A3 is an A3 domain of FVIII; C1 is a C1 domain of FVIII; S is a spacer sequence having between 1 to about 50 amino acid residues that can optionally include a cleavage sequence or amino acids compatible with restrictions sites, wherein for each occurrence, if there is any, the sequence of the spacer can be the same or different; wherein (i) a is either 0 or 1; (ii) b is either 0 or 1; (iii) c is either 0 or 1; (iv) d is either 0 or 1; (v) e is either 0 or 1; (vi) v is either 0 or 1; (vii) w is 0 or 1; (viii) x is either 0 or 1; (ix) y is either 0 or 1; and (x) z is either 0 or 1, with the proviso that v+w+x+y+z≧1. In one embodiment, the A3 domain comprises an a3 acidic region or a portion thereof. In another embodiment, at least one XTEN is inserted within the a3 acidic region or the portion thereof, N-terminus of the a3 acidic region or the portion thereof, C-terminus of the a3 acidic region or the portion thereof, or a combination thereof. In other embodiments, the factor VIII polypeptide further comprises C2 domain. In certain embodiments, at least one XTEN is inserted within the C2 domain, N-terminus of C2 domain, C-terminus of C2 domain, or a combination thereof. In still other embodiments, the Factor VIII comprises all or portion of B domain. In yet other embodiments, at least one XTEN is inserted within all or a portion of B domain, N-terminus of B domain, C-terminus of B domain, or a combination thereof.

2. CFXTEN Fusion Protein Configurations with Internal XTEN

In another aspect, the invention provides CFXTEN configured with one or more XTEN sequences located internal to the FVIII sequence. In one embodiment, invention provides CFXTEN configured with one or more XTEN sequences located internal to the FVIII sequence to confer increased stability and resistance to proteases and/or clearance mechanisms, including but not limiting to interaction with clearance receptors, compared to FVIII without the incorporated XTEN.

The invention contemplates that different configurations or sequence variants of FVIII can be utilized as the platform into which one or more XTEN are inserted. These configurations include, but are not limited to, native FVIII, FVIII BDD, and single chain FVIII (scFVIII), and variants of those configurations. In the case of scFVIII, the invention provides CFXTEN that can be constructed by replacing one or multiple amino acids of the processing site of FVIII. In one embodiment, the scFVIII is created by replacing the R1648 in the sequence RHQREITR with glycine or alanine to prevent proteolytic processing to the heterodimer form. In some embodiments, the invention provides CFXTEN comprising scFVIII wherein parts of the sequence surrounding the R1648 processing site are replaced with XTEN, as illustrated in FIGS. 10A and 10B. In one embodiment, at least about 60%, or about 70%, or about 80%, or about 90%, or about 95%, or about 97% or more of the B-domain is replaced with an XTEN sequence disclosed herein, including one or more of the R740, R1648, or R1689 cleavage sites. In another embodiment, the CFXTEN has the sequence of the B-domain between the FXIa cleavage sites at R740 and R1689 (with at least 1-5 adjacent B-domain amino acids also retained between the cut site and the start of the XTEN to permit the protease to access the cut site) replaced with XTEN. In another embodiment, the CFXTEN has the sequence of the B-domain between the FXIa cleavage site at N745 and P1640 replaced with XTEN. In other embodiments, the invention provides CFXTEN FVIII BDD sequence variants in which portions of the B-domain are deleted but only one of the FXI R740 or R1689 activation sites (and 1-5 adjacent amino acids of the B-domain) are left within the construct, wherein the XTEN remains attached at one end to either the light or heavy chain after cleavage by FXIa, as illustrated in FIGS. 5B and 5D. In one embodiment of the foregoing, the CFXTEN comprises a FVIII BDD sequence in which the amino acids between N745 to P1640 or between 5743 to Q1638 are deleted and an XTEN sequence is linked between these amino acids, connecting the heavy and light chains, and can further comprise additional XTEN inserted either in external surface loops, between FVIII domains, or at the N- or C-termini of the FVIII BDD sequence, such as one or more insertion sites from Table 5 or Table 25, or those illustrated in FIG. 7. In another embodiment of the foregoing, the CFXTEN comprises a FVIII BDD sequence in which the amino acids between K713 to Q1686 or between residues 741 and 1648 are deleted and an XTEN linked between the two amino acids, and additional XTEN can be inserted either in surface loops, between FVIII domains, or at the N- or C-termini of the FVIII BDD sequence, including but not limited to one or more insertion sites from Table 5 or Table 25. In some embodiments such CFXTEN sequences can have one or more XTEN exhibiting at least about 80%, or at least about 90%, or at least about 95%, or at least about 96%, or at least about 97%, or at least about 98%, or at least about 99%, or 100% sequence identity to an XTEN sequence from any one of Tables 4 and 9-13.

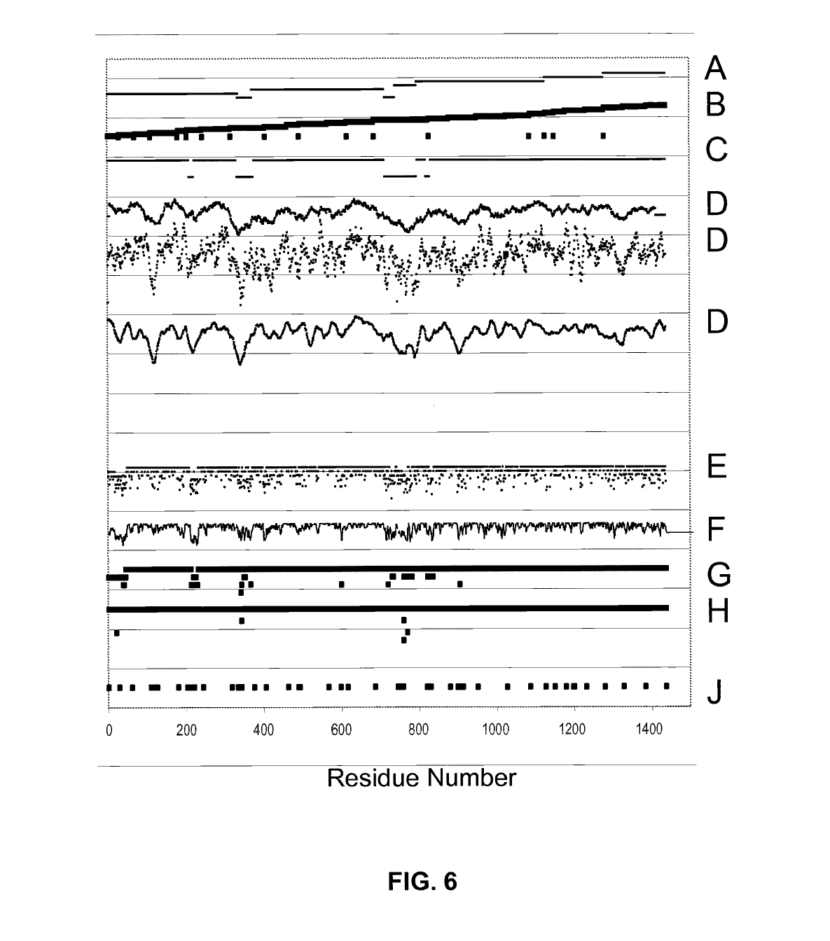

The invention contemplates other CFXTEN with internal XTEN in various configurations; schematics of exemplary configurations are illustrated in FIGS. 5 and 10. The regions suitable for XTEN insertion sites include the known domain boundaries of FVIII, exon boundaries, known surface (external) loops and solvent accessible surface area sites identified by X-ray crystallography analysis, and structure models derived from molecular dynamic simulations of FVIII, regions with a low degree of order (assessed by programs described in FIG. 6 legend), regions of low homology/lack of conservation across different species, and hydrophilic regions. In another embodiment, XTEN insertion sites were selected based on FVIII putative clearance receptor binding sites. In another embodiment, CFXTEN comprises XTEN inserted at locations not within close proximity to mutations implicated in hemophilia A listed in the Haemophilia A Mutation, Search, Test and Resource Site (HAMSTeRS) database were eliminated (Kemball-Cook G, et al. The factor VIII Structure and Mutation Resource Site: HAMSTeRS version 4. Nucleic Acids Res. (1998) 26(1):216-219). In another embodiment, potential sites for XTEN insertion include residues within FVIII epitopes that are capable of being bound by anti-FVIII antibodies occurring in sensitized hemophiliacs and that do not otherwise serve as protein interactive sites. Regions and/or sites that are considered for exclusion as XTEN insertion sites include residues/regions of factor VIII that are important in various interactions including other clotting proteins, residues surrounding each arginine activating/inactivating cleavage site acted on by the proteases thrombin, factor Xa, activated protein C, residues surrounding the signal peptide processing site (residue 1) if the construct contains the signal peptide, regions known to interact with other proteins such as FIXa, FX/FXa, thrombin, activated protein C, protein S cofactor to Protein C, von Willebrand factor, sites known to interact with phospholipid cofactors in coagulation, residues involved in domain interactions, residues coordinating Ca⁺⁺ or Cu⁺⁺ ions, cysteine residues involved in S—S intramolecular bonds, documented amino acid insertion and point mutation sites in FVIII produced in hemophilia A subjects affecting procoagulant activity, and mutation sites in FVIII made in a research lab that affect procoagulant activity. Sites considered for either insertion (to prolong half-life) or for exclusion (needed to remove spent FVIIIa or FXa) include regions known to interact with heparin sulfate proteoglycan (HSPG) or low-density lipoprotein receptor-related protein (LPR).

By analysis of the foregoing criteria, different insertion sites across the FVIII BDD sequence have been identified as candidates for insertion of XTEN, non-limiting examples of which are listed in Table 5, Table 25, and are shown schematically in FIGS. 6 and 7. In one embodiment, CFXTEN comprise XTEN insertions between the individual domains of FVIII, i.e., between the A1 and A2, or between the A2 and the B, or between the B and the A3, or between the A3 and the C1, or between the C1 and the C2 domains. In another embodiment, CFXTEN comprises XTEN inserted within the B domain or between remnant residues of the BDD sequence. In another embodiment, CFXTEN comprises XTEN inserted at known exon boundaries of the encoding FVIII gene as exons represent evolutionary conserved sequence modules that have a high probability of functioning in the context of other protein sequences. In another embodiment, CFXTEN comprise XTEN inserted within surface loops identified by the x-ray structure of FVIII. In another embodiment, CFXTEN comprise XTEN inserted within regions of low order identified as having low or no detected electron density by X-ray structure analysis. In another embodiment, CFXTEN comprise XTEN inserted within regions of low order, predicted by structure prediction algorithms such as, but not limited to FoldIndex, RONN, and Kyte & Doolitlle algorithms. In another embodiment, CFXTEN comprise XTEN inserted within sequence areas of high frequency of hydrophilic amino acids. In another embodiment, CFXTEN comprise XTEN inserted within epitopes capable of being bound by naturally-occurring anti-FVIII antibodies in sensitized hemophiliacs. In another embodiment, CFXTEN comprise XTEN inserted within sequence areas of low sequence conservation and/or differences in sequence segment length across FVIII sequences from different species. In another embodiment, CFXTEN comprise XTEN linked to the N-terminus and/or C-terminus. In another embodiment, the invention provides CFXTEN configurations with inserted XTEN selected from two or more of the criteria from the embodiments listed above. In another embodiment, the invention provides CFXTEN configurations with at least one, alternatively at least two, alter natively at least three, alternatively at least four, alternatively at least five or more XTEN inserted into a factor VIII sequence wherein the points of insertion are at or proximal to the N- or C-terminus side of the at least one, two, three, four, or five or more amino acids selected from the insertion residue amino acids of Table 5 or Table 25, or alternatively within one, or within two, or within three, or within four, or within five, or within six amino acids of the insertion residue amino acids from Table 5 or Table 25, or within the various spans of the insertion residue amino acids schematically portrayed for an exemplary FVIII BDD sequence in FIG. 7. For clarity, an XTEN inserted internal to the FVIII sequence in the foregoing embodiments is linked at its N- and C-termini to the adjoining FVIII amino acids such that the resulting CFXTEN is expressed as a linear, monomeric fusion protein (prior to any post-translational modification).

As described above, the one or more internally-located XTEN or a fragment of XTEN can have a sequence length of 6 to 1000 or more amino acid residues. In some embodiments, wherein the CFXTEN have one or two or three or four or five or more XTEN sequences internal to the FVIII, the XTEN sequences can be identical or can be different. In one embodiment, each internally-located XTEN has at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity compared to comparable lengths or fragments of XTEN selected from any one of Tables 3, 4, and 9-13, when optimally aligned. In another embodiment, the invention provides a CFXTEN configured with one or more XTEN inserted internal to a FVIII BDD sequence of Table 1 or Table 31 according to or proximal to the insertion points indicated in Table 5 or Table 25 or as illustrated in FIG. 7, as described herein. It will be understood by those of skill in the art that an XTEN inserted within the FVIII sequence at an insertion point of Table 5 or Table 25 is linked by its N- and C-termini to flanking FVIII amino acids (or via spacer or cleavage sequences, as described above), while an XTEN linked to the N- or C-terminus of FVIII would only be linked to a single FVIII amino acid (or to a spacer or cleavage sequence amino acid, as described above). By way of example only, a CFXTEN with three internal XTEN could have XTEN incorporated between FVIII BDD residues R29 and F30 (between the N-terminus of residue number 29 and the C-terminus of residue 30; i.e., insertion site no. 6 of Table 5), G182 and S183 (insertion site no. 9 of Table 5) and G1981 and V1982 (insertion site no. 39). In a variation of the foregoing embodiment, the CFXTEN with a BDD FVIII and the one or more internal XTEN has an additional XTEN located at or proximal to (e.g., within 6 amino acids) the N- and/or C-terminus of the FVIII sequence wherein each XTEN has at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity compared to an XTEN selected from any one of Tables 4, and 9-13. In the foregoing fusion protein embodiments hereinabove described in this paragraph, the CFXTEN fusion protein can further comprise one or more cleavage sequence from Table 7 or other sequences known in the art, the cleavage sequence being located between or within 6 amino acid residues of the intersection of the FVIII and the XTEN sequences, which may include two cleavage sequences in a given internal XTEN sequence. In one embodiment, the CFXTEN comprising cleavage sequences has two identical cleavage sequences, each located at or near the respective ends of one or more internal XTEN such that the XTEN is released from the fusion protein when cleaved by the protease that binds to and cleaves that sequence. The sequences that can be cleaved are described more fully below and exemplary sequences are provided in Table 7.

TABLE 5
Insertion locations for XTEN linked to the
FVIII BDD sequence

| | XTEN | | FVIII BDD | |
|---|---|---|---|---|
| | Insertion | Insertion | Downstream | FVIII |
| No. | Point* | Residue** | Sequence*** | Domain |
| 1 | 1 | A | TRR | A1 |
| 2 | 28 | A | RFP | A1 |
| 3 | 61 | I | AKP | A1 |
| 4 | 111 | G | AEY | A1 |
| 5 | 128 | V | FPG | A1 |
| 6 | 182 | G | SLA | A1 |
| 7 | 205 | G | KSW | A1 |
| 8 | 211 | E | TKN | A1 |
| 9 | 223 | A | SAR | A1 |
| 10 | 244 | G | LIG | A1 |
| 11 | 318 | D | GME | A1 |
| 12 | 334 | Q | LRM | A1 |
| 13 | 345 | D | YDD | A1 |
| 14 | 376 | K | KHP | A2 |
| 15 | 405 | R | SYK | A2 |
| 16 | 463 | I | IFK | A2 |
| 17 | 493 | K | GVK | A2 |
| 18 | 566 | I | MSD | A2 |
| 19 | 598 | P | AGV | A2 |
| 20 | 616 | S | ING | A2 |
| 21 | 686 | G | LWI | A2 |
| 22 | 1640 | P | PVL | B |
| 23 | 1652 | R | TTL | B |
| 24 | 1713 | S | SPH | A3 |
| 25 | 1724 | S | GSV | A3 |
| 26 | 1773 | V | TFR | A3 |
| 27 | 1793 | E | EDQ | A3 |
| 28 | 1799 | G | AEP | A3 |
| 29 | 1808 | K | PNE | A3 |
| 30 | 1844 | E | KDV | A3 |
| 31 | 1920 | A | ING | A3 |
| 32 | 1981 | G | VFE | A3 |
| 33 | 2020 | K | CQT | C1 |
| 34 | 2044 | G | QWA | C1 |
| 35 | 2073 | V | DLL | C1 |
| 36 | 2093 | F | SSL | C1 |
| 37 | 2125 | V | FFG | C1 |
| 38 | 2173 | S | CSM | C2 |
| 39 | 2223 | V | NNP | C2 |
| 40 | 2278 | G | KVK | C2 |
| 41 | 2332 | Y | C terminus of FVIII | C2 |

*Indicates an insertion point for XTEN based on the amino acid number of the mature FVIII protein, wherein the insertion could be either on the N- or C-terminal side of the indicated amino acid
**N-terminus residue side of the insertion point, excepting site no. 1
***The 3 amino acids of FVIII BDD sequence downstream from the insertion site (that would be joined to the C-terminus of the inserted XTEN sequence

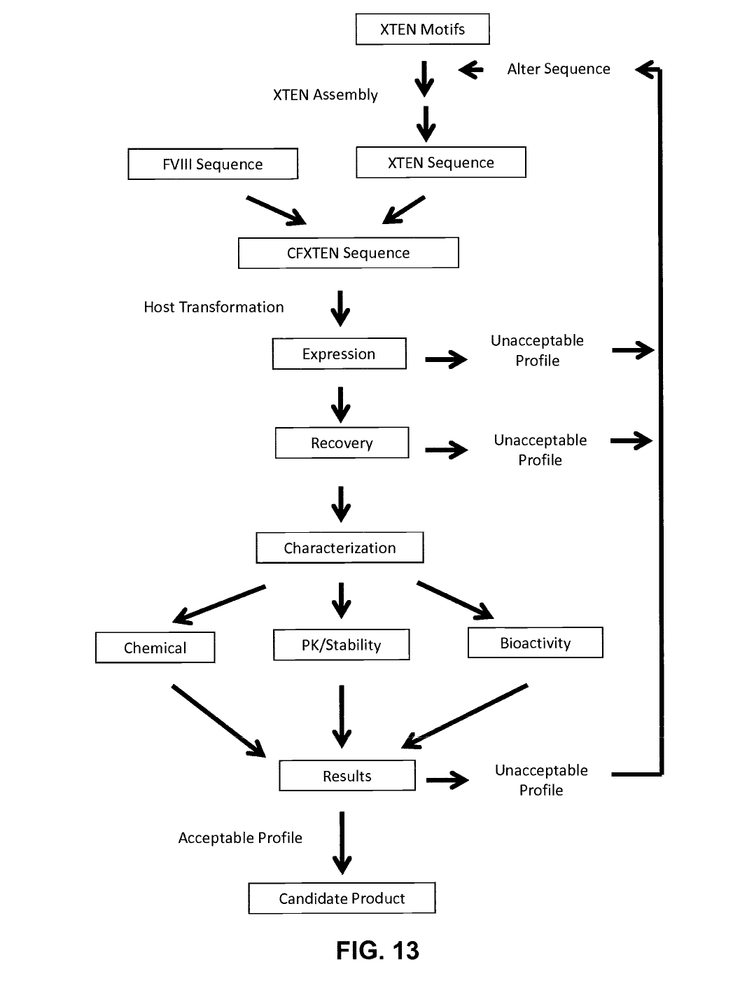

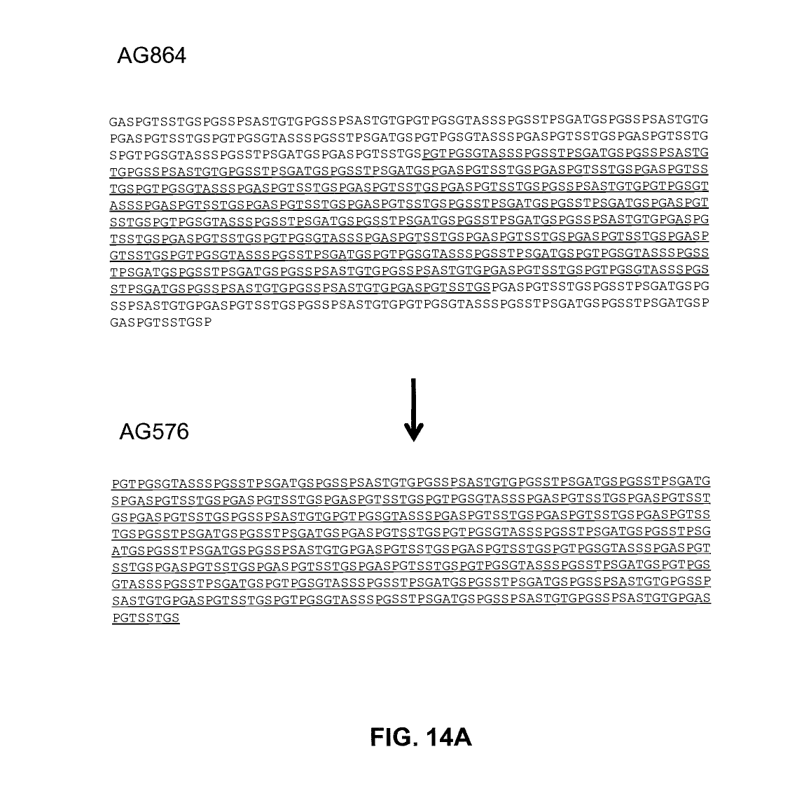

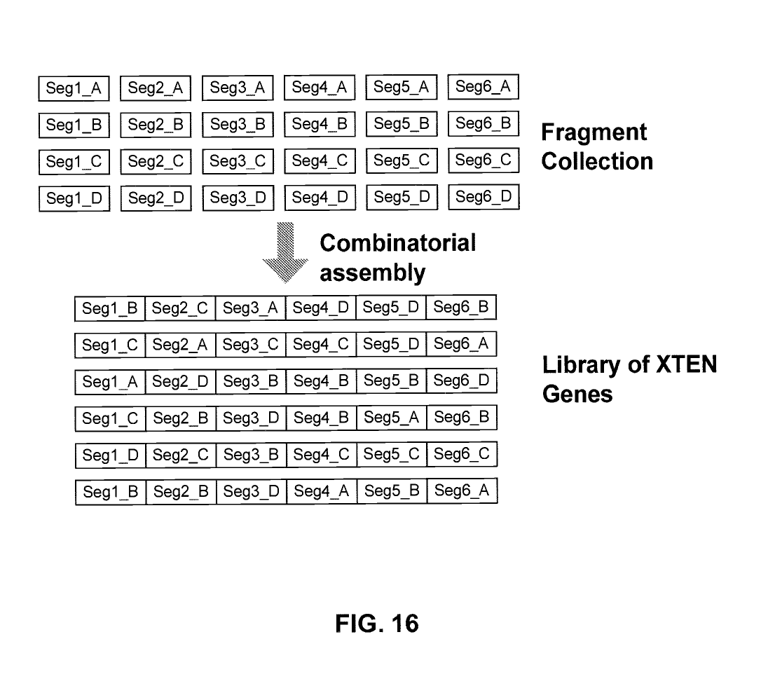

In another aspect, the invention provides libraries of components and methods to create the libraries derived from nucleotides encoding FVIII segments, XTEN, and FVIII segments linked to XTEN that are useful in the preparation of genes encoding the subject CFXTEN. In a first step, a library of genes encoding FVIII and XTEN inserted into the various single sites at or within 1-6 amino acids of an insertion site identified in Table 5 are created, expressed, and the CFXTEN recovered and evaluated for activity and pharmacokinetics as illustrated in FIG. 13. Those CFXTEN showing enhanced properties are then used to create genes encoding a FVIII segment and the insertion site plus an XTEN, with components from each enhanced insertion represented in the library, as illustrated in FIG. 16. In one embodiment, the library components are assembled using standard recombinant techniques in combinatorial fashion, as illustrated in FIG. 16, resulting in permutations of CFXTEN with multiple internal and N- and C-terminus XTEN, that can include the insertion sites of or proximal to those Table 5 or Table 25 or as illustrated in FIG. 7. The resulting constructs would then be evaluated for activity and enhanced pharmacokinetics, and those candidates resulting in CFXTEN with enhanced properties, e.g., reduced active clearance, resistance to proteases, reduced immunogenicity, and enhance pharmacokinetics, compared to FVIII not linked to XTEN, are evaluated further.

3. CFXTEN Fusion Protein Configurations with Spacer and Cleavage Sequences

In another aspect, the invention provides CFXTEN configured with one or more spacer sequences incorporated into or adjacent to the XTEN that are designed to incorporate or enhance a functionality or property to the composition, or as an aid in the assembly or manufacture of the fusion protein compositions. Such properties include, but are not limited to, inclusion of cleavage sequence(s) to permit release of components, inclusion of amino acids compatible with nucleotide restrictions sites to permit linkage of XTEN-encoding nucleotides to FVIII-encoding nucleotides or that facilitate construction of expression vectors, and linkers designed to reduce steric hindrance in regions of CFXTEN fusion proteins.

In an embodiment, a spacer sequence can be introduced between an XTEN sequence and a FVIII component to decrease steric hindrance such that the FVIII component may assume its desired tertiary structure and/or interact appropriately with its target substrate or processing enzyme. For spacers and methods of identifying desirable spacers, see, for example, George, et al. (2003) Protein Engineering 15:871-879, specifically incorporated by reference herein. In one embodiment, the spacer comprises one or more peptide sequences that are between 1-50 amino acid residues in length, or about 1-25 residues, or about 1-10 residues in length. Spacer sequences, exclusive of cleavage sites, can comprise any of the 20 natural L amino acids, and will preferably have XTEN-like properties in that the majority of residues will be hydrophilic amino acids that are sterically unhindered such as, but not limited to, glycine (G), alanine (A), serine (S), threonine (T), glutamate (E), proline (P) and aspartate (D). The spacer can be polyglycines or polyalanines, or is predominately a mixture of combinations of glycine, serine and alanine residues. In one embodiment, a spacer sequence, exclusive of cleavage site amino acids, has about 1 to 10 amino acids that consist of amino acids selected from glycine (G), alanine (A), serine (S), threonine (T), glutamate (E), and proline (P) and are substantially devoid of secondary structure; e.g., less than about 10%, or less than about 5% as determined by the Chou-Fasman and/or GOR algorithms. In one embodiment, the spacer sequence is GPEGPS. In another embodiment, the spacer sequence is GPEGPS linked to a cleavage sequence of Table 7. In addition, spacer sequences are designed to avoid the introduction of T-cell epitopes which can, in part, be achieved by avoiding or limiting the number of hydrophobic amino acids utilized in the spacer; the determination of epitopes is described above and in the Examples.

In a particular embodiment, the CFXTEN fusion protein comprises one or more spacer sequences linked at the junction(s) between the payload FVIII sequence and the one or more XTEN incorporated into the fusion protein, wherein the spacer sequences comprise amino acids that are compatible with nucleotides encoding restriction sites. In another embodiment, the CFXTEN fusion protein comprises one or more spacer sequences linked at the junction(s) between the payload FVIII sequence and the one more XTEN incorporated into the fusion protein wherein the spacer sequences comprise amino acids that are compatible with nucleotides encoding restriction sites and the amino acids and the one more spacer sequence amino acids are chosen from glycine (G), alanine (A), serine (S), threonine (T), glutamate (E), and proline (P). In another embodiment, the CFXTEN fusion protein comprises one or more spacer sequences linked at the junction(s) between the payload FVIII sequence and one more XTEN incorporated into the fusion protein wherein the spacer sequences comprise amino acids that are compatible with nucleotides encoding restriction sites and the one more spacer sequences are chosen from the sequences of Table 6. The exact sequence of each spacer sequence is chosen to be compatible with cloning sites in expression vectors that are used for a particular CFXTEN construct. In one embodiment, the spacer sequence has properties compatible with XTEN. In one embodiment, the spacer sequence is GAGSPGAETA. For XTEN sequences that are incorporated internal to the FVIII sequence, each XTEN would generally be flanked by two spacer sequences comprising amino acids compatible with restriction sites, while XTEN attached to the N- or C-terminus would only require a single spacer sequence at the junction of the two components and another at the opposite end for incorporation into the vector. As would be apparent to one of ordinary skill in the art, the spacer sequences comprising amino acids compatible with restriction sites that are internal to FVIII could be omitted from the construct when an entire CFXTEN gene is synthetically generated.

TABLE 6
Spacer Sequences Compatible with
Restriction Sites

| Spacer Sequence | Restriction Enzyme |
|---|---|
| GSPG | BsaI |
| ETET | BsaI |
| PGSSS | BbsI |
| GAP | AscI |
| GPA | FseI |
| GPSGP | SfiI |
| AAA | SacII |
| TG | AgeI |
| GT | KpnI |
| GAGSPGAETA | SfiI |

In another aspect, the present invention provides CFXTEN configurations with cleavage sequences incorporated into the spacer sequences. In some embodiments, spacer sequences in a CFXTEN fusion protein composition comprise one or more cleavage sequences, which are identical or different, wherein the cleavage sequence may be acted on by a protease, as shown in FIG. 10, to release FVIII, a FVIII component (e.g., the B domain) or XTEN sequence(s) from the fusion protein. In one embodiment, the incorporation of the cleavage sequence into the CFXTEN is designed to permit release of the FVIII component that becomes active or more active (with respect to its ability serve as a membrane binding site for factors IXa and X) upon its release from the XTEN. In the foregoing embodiment, the procoagulant activity of FVIII component of the CFXTEN is increased after cleavage by at least 30%, or at least 40%, or at least 50%, or at least 60%, or at least 70%, or at least 80%, or at least 90% compared to the intact CFXTEN. The cleavage sequences are located sufficiently close to the FVIII sequences, generally within 18, or within 12, or within 6, or within 2 amino acids of the FVIII sequence, such that any remaining residues attached to the FVIII after cleavage do not appreciably interfere with the activity (e.g., such as binding to a clotting protein) of the FVIII, yet provide sufficient access to the protease to be able to effect cleavage of the cleavage sequence. In some cases, the CFXTEN comprising the cleavage sequences will also have one or more spacer sequence amino acids between the FVIII and the cleavage sequence or the XTEN and the cleavage sequence to facilitate access of the protease; the spacer amino acids comprising any natural amino acid, including glycine, serine and alanine as preferred amino acids. In one embodiment, the cleavage site is a sequence that can be cleaved by a protease endogenous to the mammalian subject such that the CFXTEN can be cleaved after administration to a subject. In such case, the CFXTEN can serve as a prodrug or a circulating depot for the FVIII. In a particular construct of the foregoing, the CFXTEN would have one or two XTEN linked to the N- and/or the C-terminus of a FVIII-BDD via a cleavage sequence that can be acted upon by an activated coagulation factor, and would have an additional XTEN located between the processing amino acids of the B-domain at position R740 and R1689 such that the XTEN could be released, leaving a form of FVIII similar to native activated FVIII. In one embodiment of the foregoing construct, the FVIII that is released from the fusion protein by cleavage of the cleavage sequence exhibits at least about a two-fold, or at least about a three-fold, or at least about a four-fold, or at least about a five-fold, or at least about a six-fold, or at least about a eight-fold, or at least about a ten-fold, or at least about a 20-fold increase in activity compared to the intact CFXTEN fusion protein.

Examples of cleavage sites contemplated by the invention include, but are not limited to, a polypeptide sequence cleavable by a mammalian endogenous protease selected from FXIa, FXIIa, kallikrein, FVIIIa, FVIIIa, FXa, Ella (thrombin), Elastase-2, granzyme B, MMP-12, MMP-13, MMP-17 or MMP-20, or by non-mammalian proteases such as TEV, enterokinase, PreScission™ protease (rhinovirus 3C protease), and sortase A. Sequences known to be cleaved by the foregoing proteases and others are known in the art. Exemplary cleavage sequences contemplated by the invention and the respective cut sites within the sequences are presented in Table 7, as well as sequence variants thereof. For CFXTEN comprising incorporated cleavage sequence(s), it is generally preferred that the one or more cleavage sequences are substrates for activated clotting proteins. For example, thrombin (activated clotting factor II) acts on the sequence LTPRSLLV [Rawlings N. D., et al. (2008) Nucleic Acids Res., 36: D320], which is cut after the arginine at position 4 in the sequence. Active FIIa is produced by cleavage of FII by FXa in the presence of phospholipids and calcium and is down stream from factor VIII in the coagulation pathway. Once activated, its natural role in coagulation is to cleave fibrinogen, which then in turn, begins clot formation. FIIa activity is tightly controlled and only occurs when coagulation is necessary for proper hemostasis. By incorporation of the LTPRSLLV sequence into the CFXTEN between and linking the FVIII and the XTEN components, the XTEN is removed from the adjoining FVIII concurrent with activation of either the extrinsic or intrinsic coagulation pathways when coagulation is required physiologically, thereby selectively releasing FVIII. In another embodiment, the invention provides CFXTEN with incorporated FXIa cleavage sequences between the FVIII and XTEN component(s) that are acted upon only by initiation of the intrinsic coagulation system, wherein a procoagulant form of FVIII is released from XTEN by FXIa to participate in the coagulation cascade. While not intending to be bound by any particular theory, it is believed that the CFXTEN of the foregoing embodiment would sequester the FVIII away from the other coagulation factors except at the site of active clotting, thus allowing for larger doses (and therefore longer dosing intervals) with minimal safety concerns.

Thus, cleavage sequences, particularly those susceptible to the procoagulant activated clotting proteins listed in Table 7, would provide for sustained release of FVIII that, in certain embodiments of the CFXTEN, can provide a higher degree of activity for the FVIII component released from the intact form of the CFXTEN, as well as additional safety margin for high doses of CFXTEN administered to a subject. In one embodiment, the invention provides CFXTEN comprising one or more cleavage sequences operably positioned to release the FVIII from the fusion protein upon cleavage, wherein the one or more cleavage sequences has at least about 86%, or at least about 92%, or 100% sequence identity to a sequence selected from Table 7. In another embodiment, the CFXTEN comprising a cleavage sequence would have at least about 80%, or at least about 85%, or at least about 90%, or at least about 95%, or at least about 96%, or at least about 97%, or at least about 98%, or at least about 99% sequence identity compared to a sequence selected from Table 30.

In some embodiments, only the two or three amino acids flanking both sides of the cut site (four to six amino acids total) are incorporated into the cleavage sequence that, in turn, is incorporated into the CFXTEN of the embodiments, providing, e.g., XTEN release sites. In other embodiments, the incorporated cleavage sequence of Table 7 can have one or more deletions or insertions or one or two or three amino acid substitutions for any one or two or three amino acids in the known sequence, wherein the deletions, insertions or substitutions result in reduced or enhanced susceptibility but not an absence of susceptibility to the protease, resulting in an ability to tailor the rate of release of the FVIII from the XTEN. Exemplary substitutions within cleavage sequences that are utilized in the CFXTEN of the invention are shown in Table 7.

TABLE 7
Protease Cleavage Sequences

| Protease Acting Upon | Exemplary Cleavage | |
|---|---|---|
| Sequence | Sequence | Minimal Cut Site* |
| FXIa | KLTR↓AET | KD/FL/T/R↓VA/VE/GT/GV |
| FXIa | DFTR↓VVG | KD/FL/T/R↓VA/VE/GT/GV |
| FXIIa | TMTR↓IVGG | NA |
| Kallikrein | SPFR↓STGG | —/—/FL/RY↓SR/RT/—/— |
| FVIIa | LQVR↓IVGG | NA |
| FIXa | PLGR↓IVGG | —/—/G/R↓—/—/—/— |
| FXa | IEGR↓TVGG | IA/E/GFP/R↓STI/VFS/—/G |
| FIIa (thrombin) | LTPR↓SLLV | —/—/PLA/R↓SAG/—/—/— |
| Elastase-2 | LGPV↓SGVP | —/—/—/VIAT↓—/—/—/— |
| Granzyme-B | VAGD↓SLEE | V/—/—/D↓—/—/—/— |
| MMP-12 | GPAG↓LGGA | G/PA/—/G↓L/—/G/— |
| MMP-13 | GPAG↓LRGA | G/P/—/G↓L/—/GA/— |
| MMP-17 | APLG↓LRLR | —/PS/—/—↓LQ/—/LT/— |
| MMP-20 | PALP↓LVAQ | NA |
| TEV | ENLYFQ↓G | ENLYFQ↓G/S |
| Enterokinase | DDDK↓IVGG | DDDK↓IVGG |
| Protease 3C | LEVLFQ↓GP | LEVLFQ↓GP |
| (PreScission ™) | | |
| Sortase A | LPKT↓GSES | L/P/KEAD/T↓G/—/EKS/S |

↓indicates cleavage site
NA: not applicable
*the listing of multiple amino acids before, between, or after a slash indicate alternative amino acids that can be substituted at the position;
“—” indicates that any amino acid may be substituted for the corresponding amino acid indicated in the middle column

4. Exemplary CFXTEN Fusion Protein Sequences

Non-limiting examples of sequences of fusion proteins containing a single FVIII linked to a single XTEN, either joined at the N- or C-terminus are presented in Tables 14 and 28. Non-limiting examples of sequences of fusion proteins containing a single FVIII with XTEN incorporated internally to the FVIII sequence are presented in Tables 14 and 29, which may include one or two terminal XTEN. In one embodiment, a CFXTEN composition comprises a fusion protein having at least about 80% sequence identity compared to a CFXTEN from Table 14, Table 28 or Table 29, alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared to a CFXTEN from Table 14, Table 28 or Table 29, when optimally aligned. However, the invention also contemplates substitution of any of the FVIII sequences of Table 1 or Table 31 for a FVIII component of the CFXTEN of Table 14, 24 or Table 29, and/or substitution of any sequence of any one of Tables 3, 4, and 9-13 for an XTEN component of the CFXTEN of Tables 14, 28 or 29. Generally, the resulting CFXTEN of the foregoing examples retain at least a portion of the procoagulant activity of the corresponding CF not linked to the XTEN. In the foregoing fusion proteins hereinabove described in this paragraph, the CFXTEN fusion protein can further comprise one or more cleavage sequences; e.g., a sequence from Table 7, the cleavage sequence being located between the CF and the XTEN or between adjacent FVIII domains linked by XTEN. In some embodiments comprising cleavage sequence(s), the intact CFXTEN composition has less activity but a longer half-life in its intact form compared to a corresponding FVIII not linked to the XTEN, but is designed such that upon administration to a subject, the FVIII component is gradually released from the fusion protein by cleavage at the cleavage sequence(s) by endogenous proteases, whereupon the FVIII component exhibits procoagulant activity, i.e., the ability to effectively bind to and activate its target coagulation protein substrate. In non-limiting examples, the CFXTEN with a cleavage sequence has about 80% sequence identity compared to a sequence from Table 30, or about 85%, or about 90%, or about 95%, or about 97%, or about 98%, or about 99% sequence identity compared to a sequence from Table 30. However, the invention also contemplates substitution of any of the FVIII sequences of Table 1 or Table 31 for a FVIII component of the CFXTEN of Table 30, substitution of any sequence of any one of Tables 3, 4, and 9-13 for an XTEN component of the CFXTEN of Table 30, and substitution of any cleavage sequence of Table 7 for a cleavage component of the CFXTEN of Table 30. In some cases, the CFXTEN of the foregoing embodiments in this paragraph serve as prodrugs or a circulating depot, resulting in a longer terminal half-life compared to FVIII not linked to the XTEN. In such cases, a higher concentration of CFXTEN can be administered to a subject to maintain therapeutic blood levels for an extended period of time compared to the corresponding FVIII not linked to XTEN because a smaller proportion of the circulating composition is active.

The CFXTEN compositions of the embodiments can be evaluated for activity using assays or in vivo parameters as described herein (e.g., in vitro coagulation assays, assays of Table 27, or a pharmacodynamic effect in a preclinical hemophilia model or in clinical trials in humans, using methods as described in the Examples or other methods known in the art for assessing FVIII activity) to determine the suitability of the configuration or the FVIII sequence variant, and those CFXTEN compositions (including after cleavage of any incorporated XTEN-releasing cleavage sites) that retain at least about 30%, or about 40%, or about 50%, or about 55%, or about 60%, or about 70%, or about 80%, or about 90%, or about 95% or more activity compared to native FVIII sequence are considered suitable for use in the treatment of FVIII-related diseases, disorder or conditions.

Exemplary Embodiments of CFXTEN

The following are non-limiting examples of the invention:

Item 1. An isolated fusion protein comprising at least one extended recombinant polypeptide (XTEN), wherein said fusion protein having a structure of formula VIII:

(XTEN)u-(S)a-(A1)-(S)b-(XTEN)v-(S)b-(A2)-(B1)-(S)c-(XTEN)w-(S)c-(B2)-(A3)-(S)d-(XTEN)x-(S)d-(C1)-(S)e-(XTEN)y-(S)e-(C2)-(S)f-(XTEN)z VIII

wherein independently for each occurrence,

a) A1 is an A1 domain of FVIII;

b) A2 is an A2 domain of FVIII;

c) B1 is a fragment of the N-terminal end of the 13 domain having amino acid residues from residue number 740 to about number 745 of a native FVIII sequence;

d) 132 is a fragment of the C-terminal end of the 13 domain having amino acid residues from about residue numbers 1640 to number 1689 of a native FVIII sequence;

e) A3 is an A3 domain of FVIII;

f) C1 is a C1 domain of FVIII;

g) C2 is a C2 domain of FVIII;

h) S is a spacer sequence having between 1 to about 50 amino acid residues that can optionally include a cleavage sequence or amino acids compatible with restrictions sites, wherein for each occurrence, if there is any, the sequence of the spacer can be the same or different;

i) a is either 0 or 1;

j) b is either 0 or 1;

k) c is either 0 or 1;

l) d is either 0 or 1;

m) e is either 0 or 1;

n) f is either 0 or 1;

o) u is either 0 or 1;

p) v is either 0 or 1;

q) w is 0 or 1;

r) x is either 0 or 1;

s) y is either 0 or 1;

t) z is either 0 or 1, with the proviso that u+v+w+x+y+z>1; and wherein the at least one XTEN is characterized in that:

a. the XTEN comprises at least 36 amino acid residues;

b. the sum of glycine (G), alanine (A), serine (S), threonine (T), glutamate (E) and proline (P) residues constitutes more than about 80% of the total amino acid residues of the XTEN;

c. the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

d. the XTEN has greater than 90% random coil formation as determined by GOR algorithm;

e. the XTEN has less than 2% alpha helices and 2% beta-sheets as determined by Chou-Fasman algorithm;

f. the XTEN lacks a predicted T-cell epitope when analyzed by TEPITOPE algorithm, wherein the TEPITOPE threshold score for said prediction by said algorithm has a threshold of −9.

Item 2. The isolated fusion protein of item 1, comprising at least two XTENs, wherein the cumulative length of the XTENs is between about 100 to about 3000 amino acid residues.
Item 3. The isolated fusion protein of item 2, wherein each XTEN exhibits at least 90% sequence identity to a sequence of comparable length from any one of Table 4, Table 9, Table 10, Table 11, Table 12, and Table 13, when optimally aligned.
Item 4. The isolated fusion protein of any one of items 1-3, wherein the optional cleavage sequence(s) are cleavable by a mammalian protease selected from the group consisting of factor XIa, factor XIIa, kallikrein, factor VIIa, factor IXa, factor Xa, factor IIa (thrombin), Elastase-2, MMP-12, MMP13, MMP-17 and MMP-20, wherein upon cleavage of the cleavage sequences, at least one XTEN is cleaved from the fusion protein and the cleaved fusion protein exhibits an increase in procoagulant activity of at least about 30% compared to the uncleaved fusion protein.
Item 5. The isolated fusion protein of any one of items 1-4, wherein said fusion protein exhibits a prolonged in vitro half-life as compared to a corresponding factor VIII polypeptide lacking said XTEN.
Item 6. The isolated fusion protein of any one of items 1-5, wherein said fusion protein exhibits a terminal half-life longer than at least 48 hours when administered to a subject.
Item 7. An isolated fusion protein comprising a factor VIII polypeptide and at least one extended recombinant polypeptide (XTEN), wherein said factor VIII polypeptide comprises A1 domain, A2 domain, A3 domain, C1 domain, C2 domain and optionally all or a portion of B domain, and wherein said at least one XTEN is linked to said factor VIII polypeptide at (i) the C-terminus of said factor VIII polypeptide; (ii) within B domain of said factor VIII polypeptide if all or a portion of B domain is present; (iii) within the A1 domain of said factor VIII polypeptide; (iv) within the A2 domain of said factor VIII polypeptide; (v) within the A3 domain of said factor VIII polypeptide; (vi) within the C1 domain of said factor VIII polypeptide; or (vii) within the C2 domain of said factor VIII polypeptide; and wherein the XTEN is characterized in that:

a. the XTEN comprises at least 36 amino acid residues;

b. the sum of glycine (G), alanine (A), serine (S), threonine (T), glutamate (E) and proline (P) residues constitutes more than about 80% of the total amino acid residues of the XTEN;

c. the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

d. the XTEN has greater than 90% random coil formation as determined by GOR algorithm;

e. the XTEN has less than 2% alpha helices and 2% beta-sheets as determined by Chou-Fasman algorithm;

f. the XTEN lacks a predicted T-cell epitope when analyzed by TEPITOPE algorithm, wherein the TEPITOPE threshold score for said prediction by said algorithm has a threshold of −9, and wherein said fusion protein exhibits a terminal half-life that is longer than about 48 hours when administered to a subject.

Item 8. The isolated fusion protein of item 7 comprising at least another XTEN linked to said factor VIII polypeptide at the C-terminus of said factor VIII polypeptide, and within the B domain of said factor VIII polypeptide.
Item 9. The isolated fusion protein of item 7 comprising a first XTEN sequence linked to said factor VIII polypeptide at the C-terminus of said factor VIII polypeptide, and at least a second XTEN within the B domain of said factor VIII polypeptide, wherein the second XTEN is linked to the C-terminal end of about amino acid residue number 740 to about 750 and to the N-terminal end of amino acid residue numbers 1640 to about 1689 of a native FVIII sequence, wherein the cumulative length of the XTEN is at least about 100 amino acid residues.
Item 10. The isolated fusion protein of item 7 comprising at least one XTEN sequence located within B domain of said factor VIII polypeptide.
Item 11. The isolated fusion protein of item 7 comprising at least a second XTEN, wherein said at least second XTEN is linked to said factor VIII polypeptide at one or more locations selected from:

a. an insertion location from Table 5;

b. a location between any two adjacent domains of said factor VIII polypeptide, wherein said two adjacent domains are selected from the group consisting of A1 and A2 domains, A2 and B domains, B and A3 domains, A3 and C1 domains, and C1 and C2 domains;

c. the N-terminus of said factor VIII polypeptide; and

d. the C-terminus of said factor VIII polypeptide,

Item 12. The isolated fusion protein of any one of items 8-11, the second XTEN having a sequence characterized in that:

a) the XTEN comprises at least 36 amino acid residues;

b) the sum of glycine (G), alanine (A), serine (S), threonine (T), glutamate (E) and proline (P) residues constitutes more than about 80% of the total amino acid residues of the XTEN;

c) the XTEN sequence is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues does not occur more than twice in each of the sequence motifs; or (iii) the XTEN sequence has a subsequence score of less than 10;

d) the XTEN has greater than 90% random coil formation as determined by GOR algorithm;

e) the XTEN has less than 2% alpha helices and 2% beta-sheets as determined by Chou-Fasman algorithm; and

f) the XTEN lacks a predicted T-cell epitope when analyzed by TEPITOPE algorithm, wherein the TEPITOPE threshold score for said prediction by said algorithm has a threshold of −9.

Item 13. The isolated fusion protein of any one of preceding items, wherein the factor VIII polypeptide has at least 90% sequence identity compared to a sequence selected from Table 1, when optimally aligned.
Item 14. The isolated fusion protein of any one of preceding items, wherein the factor VIII polypeptide comprises human factor VIII.
Item 15. The isolated fusion protein of any one of preceding items, wherein the factor Viii polypeptide comprises a B-domain deleted variant of human factor VIII.
Item 16. The isolated fusion protein of item 11, wherein the XTEN is linked to the C-terminus of the factor VIII polypeptide.
Item 17. The isolated fusion protein of item 11, wherein the XTEN is linked to the N-terminus of the factor VIII polypeptide.
Item 18. The isolated fusion protein of any one of the preceding items, wherein the fusion protein exhibits an apparent molecular weight factor of at least about 2.
Item 19. The isolated fusion protein of any one of items 7-18, wherein the XTEN has at least 90% sequence identity compared to a sequence of comparable length selected from any one of Table 4, Table 9, Table 10, Table 11, Table 12, and Table 13, when optimally aligned.
Item 20. The isolated fusion protein of any one of items 7-18, wherein the factor VIII polypeptide is linked to the XTEN via one or two cleavage sequences that each is cleavable by a mammalian protease selected from the group consisting of factor XIa, factor XIIa, kallikrein, factor VIIa, factor IXa, factor Xa, factor IIa (thrombin), Elastase-2, MMP-12, MMP13, MMP-17 and MMP-20, wherein cleavage at the cleavage sequence by the mammalian protease releases the factor VIII sequence from the XTEN sequence, and wherein the released factor VIII sequence exhibits an increase in procoagulant activity of at least about 30% compared to the uncleaved fusion protein.
Item 21. The isolated fusion protein of item 20, wherein the cleavage sequence(s) are cleavable by factor XIa.
Item 22. The isolated fusion protein any one of items 7-21, comprising multiple XTENs located at different locations of the factor VIII polypeptide, wherein said different locations are selected from:

a. an insertion location from Table 5;

b. a location between any two adjacent domains in the factor VIII sequence, wherein said two adjacent domains are selected from the group consisting of A1 and A2, A2 and B, B and A3, A3 and C1, and C1 and C2;

c. the N-terminus of the factor VIII sequence; and

d. the C-terminus of the factor VIII sequence;

wherein the cumulative length of the multiple XTENs is at least about 100 to about 3000 amino acid residues.
Item 23. The isolated fusion protein of any one of items 7-22, wherein said fusion protein exhibits a prolonged in vitro half-life as compared to a corresponding factor VIII polypeptide lacking said XTEN.
Item 24. The isolated fusion protein of any one of items 7-23, wherein said fusion protein exhibits a terminal half-life longer than at least 48 hours when administered to a subject.
Item 25. A pharmaceutical composition comprising the fusion protein of any one of the preceding items and a pharmaceutically acceptable carrier.
Item 26. A method of treating a coagulopathy in a subject, comprising administering to said subject a composition comprising a therapeutically effective amount of the pharmaceutical composition of item 25.
Item 27. The method of item 26, wherein after said administration, a concentration of procoagulant factor VIII is maintained at about 0.05 IU/ml or more for at least 48 hours after said administration.
Item 28. The method of item 26, wherein said coagulopathy is hemophilia A.
Item 29. A method of treating a bleeding episode in a subject, comprising administering to said subject a composition comprising a therapeutically effective amount of the pharmaceutical composition of item 25, wherein the therapeutically effective amount of the fusion protein arrests a bleeding episode for a period that is at least three-fold longer compared to the corresponding factor VIII polypeptide lacking said at least one XTEN when said corresponding factor VIII is administered to a subject at a comparable dose. Item 30. A fusion protein used in the treatment of hemophilia A, comprising the fusion protein of any one of items 1-24.
Item 31. An isolated fusion protein comprising a polypeptide having at least 90% sequence identity compared to a sequence of comparable length selected from any one of Table 14, Table 28, Table 29 and Table 30.
Item 32. An isolated fusion protein comprising a factor VIII polypeptide and at least one extended recombinant polypeptide (XTEN), wherein said factor VIII polypeptide comprises A1 domain, A2 domain, A3 domain, and C1 domain, and wherein said at least one XTEN is linked to said factor VIII polypeptide at one or more insertion locations selected from the group consisting of:

a. the C-terminus of said factor VIII polypeptide;

b. within the A1 domain of said factor VIII polypeptide;

c. within the A2 domain of said factor VIII polypeptide;

d. within the A3 domain of said factor VIII polypeptide;

e. within the C1 domain of said factor VIII polypeptide;

f. one or more location between any two adjacent domains of said factor VIII polypeptide,

g. the N-terminus of said factor VIII polypeptide;

h. one or more location from FIG. 5;

i. one or more insertion location from Table 5; and

wherein the at least one XTEN is characterized in that:

i. the XTEN comprises at least 36 amino acid residues;

ii. the sum of glycine (G), alanine (A), serine (S), threonine (T), glutamate (E) and proline (P) residues constitutes more than about 80% of the total amino acid residues of the XTEN;

iii. the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

iv. the XTEN has greater than 90% random coil formation as determined by GOR algorithm;

v. the XTEN has less than 2% alpha helices and 2% beta-sheets as determined by Chou-Fasman algorithm; and

vi. the XTEN lacks a predicted T-cell epitope when analyzed by TEPITOPE algorithm, wherein the TEPITOPE threshold score for said prediction by said algorithm has a threshold of −9.

Item 33. An isolated fusion protein comprising a factor VIII polypeptide and at least one extended recombinant polypeptide (XTEN), wherein said factor VIII polypeptide comprises A1 domain, A2 domain, A3 domain, and C1 domain, and wherein said at least one XTEN is linked to said factor VIII polypeptide at one or more insertion locations from table 25 and is characterized in that:

i. the XTEN comprises at least 36 amino acid residues;

ii. the sum of glycine (G), alanine (A), serine (S), threonine (T), glutamate (E) and proline (P) residues constitutes more than about 80% of the total amino acid residues of the XTEN;

iii. the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

iv. the XTEN has greater than 90% random coil formation as determined by GOR algorithm;

v. the XTEN has less than 2% alpha helices and 2% beta-sheets as determined by Chou-Fasman algorithm; and

vi. the XTEN lacks a predicted T-cell epitope when analyzed by TEPITOPE algorithm, wherein the TEPITOPE threshold score for said prediction by said algorithm has a threshold of −9.

Item 34. The fusion protein of item 32 or 33, wherein said two adjacent domains are selected from the group consisting of the A1 and A2 domains, the A2 and A3 domains, and the A3 and C1 domains.
Item 35. The fusion protein of any one of items 32 to 34, wherein said factor VIII polypeptide further comprises C2 domain.
Item 36. The fusion protein of item 35, wherein at least one XTEN is inserted within the C2 domain, N-terminus of the C2 domain, C-terminus of the C2 domain, or a combination thereof.
Item 37. The fusion protein of any one of items 32 to 36, wherein said Factor VIII comprises a full-length B domain or a partially deleted B domain.
Item 38. The fusion protein of item 37, wherein at least one XTEN is inserted within the full-length B domain or partially deleted B domain, N-terminus of the full-length B domain or partially deleted B domain, C-terminus of the full-length B domain or partially deleted B domain, or a combination thereof.
Item 39. The fusion protein of any one of items 32 to 38, wherein said A3 domain comprises an a3 acidic region or a portion thereof.
Item 40. The fusion protein of item 27, wherein at least one XTEN is inserted within the a3 acidic region or the portion thereof, N-terminus of the a3 acidic region or the portion thereof, C-terminus of the a3 acidic region or the portion thereof, or a combination thereof.
Item 41. The fusion protein of any one of items 32 to 40, further comprising one or more spacer linked to said at least one XTEN.
Item 42. The fusion protein of item 41, wherein said spacer comprises about 1 to about 50 amino acid residues that optionally includes a cleavage sequence or amino acids compatible with restriction sites, wherein for each occurrence, if there is any, the sequence of the spacer is the same or different.
Item 43. An isolated fusion protein comprising a structure of formula (A):

(XTEN)v-(S)a-(A1)-(S)b-(XTEN)w-(S)b-(A2)-(S)c-(XTEN)x-(S)c-(A3)-(S)d-(XTEN)y-(S)d-(C1)-(S)e-(XTEN)z (A)

wherein independently for each occurrence,

u) A1 is an A1 domain of FVIII;

v) A2 is an A2 domain of FVIII;

w) A3 is an A3 domain of FVIII;

x) C1 is a C1 domain of FVIII;

y) S is a spacer sequence having between 1 to about 50 amino acid residues that optionally includes a cleavage sequence or amino acids compatible with restrictions sites, wherein for each occurrence, if there is any, the sequence of the spacer is the same or different;

wherein

(i) a is either 0 or 1;

(ii) b is either 0 or 1;

(iii) c is either 0 or 1;

(iv) d is either 0 or 1;

(v) e is either 0 or 1;

(vi) v is either 0 or 1;

(vii) w is 0 or 1;

(viii) x is either 0 or 1;

(ix) y is either 0 or 1;

(x) z is either 0 or 1,

with the proviso that v+w+x+y+z>1,
wherein said XTEN is characterized in that:

(1). the XTEN comprises at least 36 amino acid residues;

(2). the sum of glycine (G), alanine (A), serine (S), threonine (T), glutamate (E) and proline (P) residues constitutes more than about 80% of the total amino acid residues of the XTEN;

(3). the XTEN is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues do not occur more than twice in each of the non-overlapping sequence motifs; or (iii) the XTEN sequence has a subsequence score of less than 10;

(4). the XTEN has greater than 90% random coil formation as determined by GOR algorithm;

(5). the XTEN has less than 2% alpha helices and 2% beta-sheets as determined by Chou-Fasman algorithm; and

(6). the XTEN lacks a predicted T-cell epitope when analyzed by TEPITOPE algorithm, wherein the TEPITOPE threshold score for said prediction by said algorithm has a threshold of −9.

Item 44. The fusion protein of item 43, wherein said factor VIII polypeptide further comprises C2 domain.
Item 45. The fusion protein of item 44, wherein at least one XTEN is inserted within the C2 domain, N-terminus of the C2 domain, C-terminus of the C2 domain, or a combination thereof.
Item 46. The fusion protein of any one of items 43 to 45, wherein said Factor VIII comprises a full or a partially deleted B domain anywhere between the A2 and the A3.
Item 47. The fusion protein of item 46, wherein at least one XTEN is inserted within the full-length B domain or partially deleted B domain, N-terminus of the full-length B domain or partially deleted B domain, C-terminus of the full-length B domain or partially deleted B domain, or a combination thereof.
Item 48. The fusion protein of any one of items 43 to 47, wherein said A3 domain comprises an a3 acidic region or a portion thereof.
Item 49. The fusion protein of item 48, wherein at least one XTEN is inserted within the a3 acidic region or the portion thereof, N-terminus of the a3 acidic region or the portion thereof, C-terminus of the a3 acidic region or the portion thereof, or a combination thereof.
Item 50. The fusion protein of item 44, wherein at least one XTEN is further inserted within the A1, the A2, the A3, the C1, the C2, or a combination of two or more thereof.
Item 51. The fusion protein of any one of items 37-38 and 46-47, wherein said B domain comprises amino acid residues 741 to 743 of mature FVIII and/or amino acid residues 1638 to 1648 of mature FVIII.
Item 52. The fusion protein of any one of items 32 to 51, wherein said at least one XTEN is inserted right after Arginine at residue 1648 of mature FVIII.
Item 53. The fusion protein of any one of items 32 to 52, wherein said at least one XTEN is inserted in one or more thrombin cleavage site selected from the group consisting of amino acid residues 372 of FVIII, 740 of FVIII, and 1689 of FVIII.
Item 54. The fusion protein of any one of items 43 to 53, wherein the sum of v, w, x, y, and z, equals to 2, 3, 4, 5, 6, 7, 8, 9, or 10.
Item 55. The fusion protein of any one of items 32 to 54, wherein said factor VIII polypeptide comprises a heavy chain and a light chain, wherein said heavy chain comprises the A1 domain and the A2 domain, and said light chain comprises the A3 domain and the C1 domain.
Item 56. The fusion protein of item 55, wherein said heavy chain further comprises a partially deleted B domain and/or the light chain further comprises a partially deleted B domain.
Item 57. The fusion protein of any one of items 42-56, wherein the optional cleavage sequence(s) are cleavable by a mammalian protease selected from the group consisting of factor XIa, factor XIIa, kallikrein, factor VIIa, factor IXa, factor Xa, factor IIa (thrombin), Elastase-2, MMP-12, MMP13, MMP-17 and MMP-20, wherein upon cleavage of the cleavage sequences, at least one XTEN is cleaved from the fusion protein and the cleaved fusion protein exhibits an increase in procoagulant activity of at least about 30% compared to the uncleaved fusion protein.
Item 58. The fusion protein of any one of items 32 to 57, wherein one or more of said at least one XTEN is 36 amino acids, 42 amino acids, 144 amino acids, 288 amino acids, 576 amino acids, or 864 amino acids in length.
Item 59. The fusion protein of any one of items 32 to 57, wherein one or more of said at least one XTEN is selected from the group consisting of: XTEN_AE42, XTEN_AE864, XTEN_AE576, XTEN_AE288, XTEN_AE144, XTEN_AG864, XTEN_AG576, XTEN_AG288, and XTEN_AG144.
Item 60. The fusion protein of any one of items 32 to 59, which comprises at least two XTENs, wherein the cumulative length of the XTENs is between about 100 to about 3000 amino acid residues.
Item 61. The fusion protein of any one of items 32 to 60, wherein said fusion protein exhibits a prolonged in vitro half-life as compared to a corresponding factor VIII polypeptide lacking said XTEN.
Item 62. The fusion protein of any one of items 32-61, wherein said fusion protein exhibits a terminal half-life longer than at least 48 hours when administered to a subject.
Item 63. The fusion protein of any one of items 32 to 62, wherein a first XTEN of said at least one XTEN is linked to said factor VIII polypeptide at the C-terminus of said factor VIII polypeptide, and a second XTEN of said at least one XTEN is linked within the B domain of said factor VIII polypeptide.
Item 64. The fusion protein of item 63, wherein said second XTEN is linked between amino acid residue 743 and amino acid residue 1638 of mature FVIII.
Item 65. The fusion protein of item 63 or 64, wherein said first XTEN or said second XTEN has 36 amino acids, 42 amino acids, 144 amino acids, 288 amino acids, 576 amino acids, or 864 amino acids in length.
Item 66. The fusion protein of any one of items 63 to 65, wherein said first XTEN or said second XTEN is selected from the group consisting of: XTEN_AE42_{—}4, XTEN_AE864, XTEN_AE576, XTEN_AE288, XTEN_AE144, XTEN_AG864, XTEN_AG576, XTEN_AG288, and XTEN_AG144.
Item 67. The fusion protein of any one of the preceding items, wherein the cumulative length of the XTENs is at least about 100 amino acid residues.
Item 68. The fusion protein of any one of items 32 to 67, further comprising one or more XTEN linked to the factor VIII polypeptide at one or more locations selected from the group consisting of:

a. one or more insertion location from Table 5 or Table 25;

b. one or more insertion location from FIG. 5;

c. within the B domain of said factor VIII polypeptide;

d. within the A1 domain of said factor VIII polypeptide;

c. within the A2 domain of said factor VIII polypeptide;

f. within the a3 acidic region of said factor VIII polypeptide;

g. within the A3 domain of said factor VIII polypeptide;

h. within the C1 domain of said factor VIII polypeptide;

i. within the C2 domain of said factor VIII polypeptide;

j. one or more insertion location between any two adjacent domains of said factor VIII polypeptide, wherein said two adjacent domains are selected from the group consisting of A1 and A2 domains, A2 and B domains, B domain and a3 region, A2 domain and a3 region when B domain is completely deleted, a3 region and A3 domains, A3 and C1 domains, and C1 and C2 domains;

k. the N-terminus of said factor VIII polypeptide; and

l. the C-terminus of said factor VIII polypeptide.

Item 69. The fusion protein of any one of items 32 to 67, further comprising one or more XTEN linked to the factor VIII polypeptide at one or more locations from Table 25.
Item 70. The fusion protein item 68 or 69, wherein the one or more XTEN is characterized in that:

a. the XTEN comprises at least 36 amino acid residues;

b. the sum of glycine (G), alanine (A), serine (S), threonine (T), glutamate (E) and proline (P) residues constitutes more than about 80% of the total amino acid residues of the XTEN;

c. the XTEN sequence is substantially non-repetitive such that (i) the XTEN contains no three contiguous amino acids that are identical unless the amino acids are serine; (ii) at least about 80% of the XTEN sequence consists of non-overlapping sequence motifs, each of the sequence motifs comprising about 9 to about 14 amino acid residues consisting of four to six amino acids selected from glycine (G), alanine (A), serine (S), threonine (T), glutamate (E) and proline (P), wherein any two contiguous amino acid residues does not occur more than twice in each of the sequence motifs; or (iii) the XTEN sequence has a subsequence score of less than 10;

d. the XTEN has greater than 90% random coil formation as determined by GOR algorithm;

e. the XTEN has less than 2% alpha helices and 2% beta-sheets as determined by Chou-Fasman algorithm; and

f. the XTEN lacks a predicted T-cell epitope when analyzed by TEPITOPE algorithm, wherein the TEPITOPE threshold score for said prediction by said algorithm has a threshold of −9.

Item 71. The fusion protein of any one of items 68 to 70, wherein said one or more XTEN has 36 amino acids, 42 amino acids, 144 amino acids, 288 amino acids, 576 amino acids, or 864 amino acids in length.
Item 72. The fusion protein of any one of items 68 to 70, wherein said one or more XTEN is selected from the group consisting of: XTEN_AE42_{—}4, XTEN_AE864, XTEN_AE576, XTEN_AE288, XTEN_AE144, XTEN_AG864, XTEN_AG576, XTEN_AG288, and XTEN_AG144.
Item 73. The fusion protein of any one of the preceding items, wherein the factor VIII polypeptide has at least 90% sequence identity compared to a sequence selected from Table 1 or Table 31, when optimally aligned.
Item 74. The fusion protein of any one of the preceding items, wherein the factor VIII polypeptide comprises human factor VIII.
Item 75. The fusion protein of any one of the preceding items, wherein said at least one XTEN is linked to the C-terminus of the factor VIII polypeptide.
Item 76. The fusion protein of the any one of the preceding item, wherein said at least one XTEN is linked to the N-terminus of the factor VIII polypeptide.
Item 77. The fusion protein of the any one of the preceding items, wherein said at least one XTEN is linked to an insertion location from Table 25.
Item 78. The fusion protein of any one of the preceding items, wherein the fusion protein exhibits an apparent molecular weight factor of at least about 2.
Item 79. The fusion protein of any one of items the preceding items, wherein the XTEN has at least 90% sequence identity compared to a sequence of comparable length selected from any one of Table 4, Table 9, Table 10, Table 11, Table 12, and Table 13, when optimally aligned.
Item 80. The fusion protein of item 57, wherein the cleavage sequence(s) are cleavable by factor XIa.
Item 81. A pharmaceutical composition comprising the fusion protein of any one of the preceding items and a pharmaceutically acceptable carrier.
Item 82. A method of treating a coagulopathy in a subject, comprising administering to said subject a composition comprising a therapeutically effective amount of the pharmaceutical composition of item 81.
Item 83. The method of item 82, wherein after said administration, a concentration of procoagulant factor VIII is maintained at about 0.05 IU/ml or more for at least 48 hours after said administration.
Item 84. The method of item 82 or 83, wherein said coagulopathy is hemophilia A.
Item 85. A method of treating a bleeding episode in a subject, comprising administering to said subject a composition comprising a therapeutically effective amount of the pharmaceutical composition of item 82, wherein the therapeutically effective amount of the fusion protein arrests a bleeding episode for a period that is at least three-fold longer compared to the corresponding factor VIII polypeptide lacking said at least one XTEN when said corresponding factor VIII is administered to a subject at a comparable dose.
Item 86. A fusion protein used in the treatment of hemophilia A, comprising the fusion protein of any one of items 1-85.

### V). Properties of the CFXTEN Compositions of the Invention

(a) Pharmacokinetic Properties of CFXTEN

In another aspect, the present invention provides CFXTEN fusion proteins and pharmaceutical compositions comprising CFXTEN with enhanced pharmacokinetics compared to FVIII not linked to XTEN. The pharmacokinetic properties of a FVIII that can be enhanced by linking a given XTEN to the FVIII include, but are not limited to, terminal half-life, area under the curve (AUC), Cₘₐₓ, volume of distribution, maintaining the biologically active CFXTEN above a minimum effective blood unit concentration for a longer period of time compared to the FVIII not linked to XTEN, and bioavailability, as well as other properties that permit less frequent dosing or a longer-lived pharmacologic effect compared to FVIII not linked to XTEN. Enhancement of one or more of these properties can resulting benefits in the treatment of factor VIII-related disorders, and related conditions.

Exogenously administered factor VIII has been reported to have a terminal half-life in humans of approximately 12-14 hours when complexed with normal von Willebrand factor protein, whereas in the absence of von Willebrand factor, the half-life of factor VIII is reduced to 2 hours (Tuddenham E G, et al., Br J Haematol. (1982) 52(2):259-267; Bjorkman, S., et al. Clin Pharmacokinet. (2001) 40:815). As a result of the enhanced properties conferred by XTEN, the CFXTEN, when used at the dose and dose regimen determined to be appropriate for the composition by the methods described herein, can achieve a circulating concentration resulting in a desired procoagulant or clinical effect for an extended period of time compared to a comparable dose of the FVIII not linked to XTEN. As used herein, a “comparable dose” means a dose with an equivalent moles/kg or International Units/kg (IU/kg) for the composition that is administered to a subject. It will be understood in the art that a “comparable dosage” of CFXTEN fusion protein would represent a greater weight of agent but would have essentially the same IUs or mole-equivalents of FVIII in the dose of the fusion protein administered.

An international unit (“IU”) of factor VIII is defined in the art as the coagulant activity present in 1 ml of normal human plasma. A normal, non-hemophilic individual human is expected to have about 100 IU/dL factor VIII activity. In hemophilia A, the doses required to treat are dependent on the condition. For minor bleeding, doses of native or recombinant factor VIII of 20 to 40 μl/kg are typically administered, as necessary. For moderate bleeding, doses of 30 to 60 IU/kg are administered as necessary, and for major bleeding, doses of 80 to 100 IU/kg may be required, with repeat doses of 20 to 25 III/kg given every 8 to 12 hours until the bleeding is resolved. For prophylaxis against bleeding in patients with severe hemophilia A, the usual doses of native or recombinant FVIII preparations are 20 to 40 IU/kg body weight at intervals of about 2 to 3 days. A standard equation for estimating an appropriate dose of a composition comprising FVIII is:

Required units=body weight (kg)×desired factor VIII rise (IU/dL or % of normal)×0.5 (IU/kg per IU/dL).

For the inventive compositions, CFXTEN with a longer terminal half-life are generally preferred, so as to improve patient convenience, to increase the interval between doses and to reduce the amount of drug required to achieve a sustained effect. Using CFXTEN from the embodiments hereinabove described, the administration of the fusion protein results in an improvement in at least one of the parameters disclosed herein as being useful for assessing the subject diseases, conditions or disorders (e.g., resolution of a bleeding event, achieving or maintaining a minimum blood concentration in IU/ml, such as 0.01-0.05 to 0.05 to 0.4 IU/ml, and/or achieving a clotting assay result within 30% of normal) using a lower IU dose of fusion protein compared to the corresponding FVIII component not linked to the XTEN and administered at a comparable IU dose or dose regimen to a subject. In one embodiment, the total dose in IUs administered to achieve and/or maintain the improvement in at least one parameter is at least about three-fold lower, or at least about four-fold, or at least about five-fold, or at least about six-fold, or at least about eight-fold, or at least about 10-fold lower compared to the corresponding FVIII component not linked to the XTEN.

As described more fully in the Examples pertaining to pharmacokinetic characteristics of fusion proteins comprising XTEN, it was observed that increasing the length of the XTEN sequence confers a disproportionate increase in the terminal half-life of a fusion protein comprising the XTEN. Accordingly, the invention provides CFXTEN fusion proteins and pharmaceutical compositions comprising CFXTEN wherein the CFXTEN exhibits a targeted half-life for the CFXTEN composition administered to a subject. In some embodiments, the invention provides monomeric CFXTEN fusion proteins comprising one or more XTEN wherein the XTEN is selected to confer an increase in the terminal half-life for the CFXTEN administered to a subject, compared to the corresponding FVIII not linked to the XTEN and administered at a comparable dose, wherein the increase is at least about two-fold longer, or at least about three-fold, or at least about four-fold, or at least about five-fold, or at least about six-fold, or at least about seven-fold, or at least about eight-fold, or at least about nine-fold, or at least about ten-fold, or at least about 15-fold, or at least a 20-fold, or at least a 40-fold or greater an increase in terminal half-life compared to the FVIII not linked to the XTEN. In another embodiment, the administration of a therapeutically effective amount of CFXTEN or a pharmaceutical compositions comprising CFXTEN to a subject in need thereof results in a terminal half-life that is at least 12 h greater, or at least about 24 h greater, or at least about 48 h greater, or at least about 96 h greater, or at least about 144 h greater, or at least about 7 days greater, or at least about 14 days greater, or at least about 21 days greater compared to a comparable dose of FVIII not linked to XTEN. In another embodiment, administration of a therapeutically effective dose of a CFXTEN fusion protein to a subject in need thereof can result in a gain in time between consecutive doses necessary to maintain a therapeutically effective blood level of the fusion protein of at least 0.01-0.05 to about 0.1-0.4 IU/ml of at least 48 h, or at least 72 h, or at least about 96 h, or at least about 120 h, or at least about 7 days, or at least about 14 days, or at least about 21 days between consecutive doses compared to a FVIII not linked to XTEN and administered at a comparable dose. It will be understood in the art that the time between consecutive doses to maintain a “therapeutically effective blood level” will vary greatly depending on the physiologic state of the subject, and it will be appreciated that a patient with hemophilia A undergoing surgery or suffering severe trauma will require more frequent dosing of a factor VIII preparation compared to a patient receiving the same preparation for conventional prophylaxis. The foregoing notwithstanding, it is believed that the CFXTEN of the present invention permit less frequent dosing, as described above, compared to a FVIII not linked to XTEN.

In one embodiment, the present invention provides CFXTEN fusion proteins and pharmaceutical compositions comprising CFXTEN that exhibit, when administered to a subject in need thereof, an increase in AUC of at least about 50%, or at least about 60%, or at least about 70%, or at least about 80%, or at least about 90%, or at least about a 100%, or at least about 150%, or at least about 200%, or at least about 300%, or at least about 500%, or at least about 1000%, or at least about a 2000% compared to the corresponding FVIII not linked to the XTEN and administered to a subject at a comparable dose. The pharmacokinetic parameters of a CFXTEN can be determined by standard methods involving dosing, the taking of blood samples at times intervals, and the assaying of the protein using ELISA, HPLC, radioassay, clotting assays, the assays of Table 27, or other methods known in the art or as described herein, followed by standard calculations of the data to derive the half-life and other PK parameters.

The enhanced PK parameters allow for reduced dosing of the subject compositions, compared to FVIII not linked to XTEN, particularly for those subjects receiving doses for routine prophylaxis. In one embodiment, a smaller IU amount of about two-fold less, or about three-fold less, or about four-fold less, or about five-fold less, or about six-fold less, or about eight-fold less, or about 10-fold less or greater of the fusion protein is administered in comparison to the corresponding FVIII not linked to the XTEN under a dose regimen needed to maintain hemostasis or a minimum effective blood concentration (e.g., 0.01-0.5 to about 0.1-0.4 IU/ml), and the fusion protein achieves a comparable area under the curve as the corresponding IU amount of the FVIII not linked to the XTEN. In another embodiment, the CFXTEN fusion protein or a pharmaceutical compositions comprising CFXTEN requires less frequent administration for routine prophylaxis of a hemophilia A subject, wherein the dose is administered about every four days, about every seven days, about every 10 days, about every 14 days, about every 21 days, or about monthly of the fusion protein administered to a subject, and the fusion protein achieves a comparable area under the curve as the corresponding FVIII not linked to the XTEN. In yet other embodiments, an accumulative smaller IU amount of about 5%, or about 10%, or about 20%, or about 40%, or about 50%, or about 60%, or about 70%, or about 80%, or about 90% less of the fusion protein is administered to a subject in comparison to the corresponding IU amount of the FVIII not linked to the XTEN under a dose regimen needed to maintain hemostasis or a minimum effective blood concentration (e.g., 0.5 IU/ml), yet the fusion protein achieves at least a comparable area under the curve as the corresponding FVIII not linked to the XTEN. The accumulative smaller IU amount is measure for a period of at least about one week, or about 14 days, or about 21 days, or about one month.

In one aspect, the invention provides CFXTEN compositions designed to reduce active clearance of the fusion protein, thereby increasing the terminal half-life of CFXTEN administered to a subject, while still retaining procoagulant activity. it is believed that the CFXTEN of the present invention have comparatively higher and/or sustained activity achieved by reduced active clearance of the molecule by the addition of unstructured XTEN to the FVIII coagulation factor. The clearance mechanisms to remove FVIII from the circulation have yet to be fully elucidated. Uptake, elimination, and inactivation of coagulation proteins can occur in the circulatory system as well as in the extravascular space. Coagulation factors are complex proteins that interact with a large number of other proteins, lipids, and receptors, and many of these interactions can contribute to the elimination of CFs from the circulation. Factor VIII and von Willebrand factor (VWF) circulate in the blood as a tight, non-covalently linked complex in which VWF serves as a carrier that likely contributes to the protection of FVIII from active cleavage mechanisms. For example: (i) VWF stabilizes the heterodimeric structure of FVIII; (ii) VWF protects FVIII from proteolytic degradation by phospholipid-binding proteases like activated protein C and activated FX (FXa); (iii) VWF interferes with binding of FVIII to negatively charged phospholipid surfaces exposed within activated platelets; (iv) VWF inhibits binding of FVIII to activated FIX (FIXa), thereby denying FVIII access to the FX-activating complex; and (v) VWF prevents the cellular uptake of FVIII (Lenting, P. J., et al., J Thrombosis and Haemostasis (2007) 5(7):1353-1360). In addition, LDL receptor-related protein (LRP1, also known as α2-macrogobulin receptor or CD91) has been identified as a candidate clearance receptor for FVIII, with LRP1 binding sites identified on both chains of the heterodimer form of FVIII (Lenting P J, et al., J Biol Chem (1999) 274: 23734-23739; Saenko E L, et al., J Biol Chem (1999) 274: 37685-37692). LRPs are involved in the clearance of a diversity of ligands including proteases, inhibitors of the Kunitz type, protease serpin complexes, lipases and lipoproteins (Narita, et al., Blood (1998) 2:555-560). It has been shown that the light chain, but not the heavy chain, of factor VIII binds to surface-exposed LRP1 receptor protein (Lentig et al. (J Biol Chem (1999) 274(34):23734-23739; and U.S. Pat. No. 6,919,311), which suggests that LRP1 may play an essential role in the active clearance of proteins like FVIII. While the VWF-FVIII interaction is of high affinity (<1 nM), the complex is nevertheless in a dynamic equilibrium, such that a small but significant portion of the FVIII molecules (5-8%) circulate as a free protein (Leyte A, et al., Biochem J (1989) 257: 679-683; Noe D A. Haemostasis (1996) 26: 289-303). As such, a portion of native FVIII is unprotected by VWF, allowing active clearance mechanisms to remove the unprotected FVIII from the circulation.

In one embodiment, the invention provides CFXTEN that associate with VWF but have enhanced protection from active clearance receptors conferred by the incorporation of two more XTEN at one or more locations within the FVIII molecule (e.g., locations selected from Table 5 or Table 25 or FIG. 7), wherein the XTEN interfere with the interaction of the resulting CFXTEN with those clearance receptors with the result that the pharmacokinetic properties of the CFXTEN is enhanced compared to the corresponding FVIII not linked to XTEN. In another embodiment, the invention provides CFXTEN that have reduced or no binding affinity with VWF, but are nevertheless configured to have enhanced protection from active clearance receptors conferred by the incorporation of XTEN at one or more locations within the FVIII molecule, wherein the XTEN interfere with the interaction of factor VIII with those receptors. The invention provides a method wherein the CFXTEN fusion proteins created with the multiple insertions are evaluated for inhibition of binding to clearance receptors, compared to FVIII not linked to XTEN, using in vitro binding assays or in vivo pharmacokinetic models described herein or other assays known in the art, and selecting those that demonstrate reduced binding yet retain procoagulant FVIII activity. In addition, the foregoing fusion proteins can also incorporate longer XTEN lengths serving as carriers in order to achieve pharmacokinetic properties that are further enhanced. Table 5, Table 25 and FIG. 7 provide non-limiting examples of XTEN insertion points within the factor VIII sequence. Using such insertion points, the invention contemplates CFXTEN that have combinations of configurations with multiple inserted XTEN to further increase the protection against active clearance mechanisms and, hence, increase the terminal half-life of the CFXTEN. Not to be bound by a particular theory, the XTEN of the CFXTEN compositions with high net charge (e.g., CFXTEN comprising AE family XTEN) are expected, as described above, to have less non-specific interactions with various negatively-charged surfaces such as blood vessels, tissues, or various receptors, which would further contribute to reduced active clearance. Conversely, the XTEN of the CFXTEN compositions with a low (or no) net charge (e.g., CFXTEN comprising AG family XTEN) are expected to have a higher degree of interaction with surfaces that, while contributing to active clearance, can potentiate the activity of the associated coagulation factor, given the known contribution of cell (e.g., platelets) and vascular surfaces to the coagulation process and the intensity of activation of coagulation factors (Zhou, R., et al., Biomaterials (2005) 26(16):2965-2973; London, F., et al. Biochemistry (2000) 39(32):9850-9858). The invention, in part, takes advantage of the fact that certain ligands wherein reduced binding to a clearance receptor, either as a result of a decreased on-rate or an increased off-rate, may be effected by the obstruction of either the N- or C-terminus and using that terminus as the linkage to another polypeptide of the composition, whether another molecule of a CF, an XTEN, or a spacer sequence results in the reduced binding. The choice of the particular configuration of the CFXTEN fusion protein can be tested by methods disclosed herein to confirm those configurations that reduce the degree of binding to a clearance receptor such that a reduced rate of active clearance is achieved. In one embodiment, the CFXTEN comprises a FVIII-XTEN sequence that has one or more XTEN inserted at locations selected from Table 5, Table 25, or FIG. 7 wherein the terminal half-life of the CFXTEN is increased at least about two-fold, or at least about three-fold, or at least about four-fold, or at least about five-fold, or at least about six-fold, or at least about eight-fold, or at least about ten-fold, or at least about twenty-fold compared to a FVIII not linked to an XTEN. In another embodiment, the CFXTEN comprises a FVIII-XTEN sequence that has a first and at least a second XTEN inserted at a first and second location selected from Table 5, Table 25, or FIG. 7 wherein the terminal half-life of the CFXTEN is increased at least about two-fold, or at least about three-fold, or at least about four-fold, or at least about five-fold, or at least about six-fold, or at least about eight-fold, or at least about ten-fold, or at least about twenty-fold compared to a FVIII not linked to an XTEN. In yet another embodiment, the CFXTEN comprises a FVIII-XTEN sequence that incorporates multiple XTEN sequences using multiple insertion locations selected from Table 5, Table 25 or FIG. 7 wherein the terminal half-life of the CFXTEN is increased at least about two-fold, or at least about three-fold, or at least about four-fold, or at least about five-fold, or at least about six-fold, or at least about eight-fold, or at least about ten-fold, or at least about twenty-fold compared to a FVIII not linked to an XTEN. In the foregoing embodiments hereinabove described in this paragraph, the XTEN incorporated into the CFXTEN configurations can be identical or they can be different, and can have at least about 80%, or 90%, or 91%, or 92%, or 93%, or 94%, or 95%, or 96%, or 97%, or 98%, or 99%, sequence identity to a sequence from any one of Tables 3, 4, and 9-13, and can optionally include one or more cleavage sequences from Table 7, facilitating release of one or more of the XTEN from the CFXTEN fusion protein.

In one embodiment, the invention provides CFXTEN that enhance the pharmacokinetics of the fusion protein by linking one or more XTEN to the FVIII component of the fusion protein wherein the fusion protein has an increase in apparent molecular weight factor of at least about two-fold, or at least about three-fold, or at least about four-fold, or at least about five-fold, or at least about six-fold, or at least about seven-fold, or at least about eight-fold, or at least about ten-fold, or at least about twelve-fold, or at least about fifteen-fold, and wherein the terminal half-life of the CFXTEN when administered to a subject is increased at least about two-fold, or at least about four-fold, or at least about eight-fold, or at least about 10-fold or more compared to the corresponding FVIII not linked to XTEN. In the foregoing embodiment, wherein at least two XTEN molecules are incorporated into the CFXTEN, the XTEN can be identical or they can be of a different sequence composition, net charge, or length. The XTEN can have at least about 80%, or 90%, or 91%, or 92%, or 93%, or 94%, or 95%, or 96%, or 97%, or 98%, or 99%, sequence identity to a sequence from any one of Tables 3, 4, and 9-13, and can optionally include one or more cleavage sequences from Table 7, facilitating release of one or more of the XTEN from the CFXTEN fusion protein.

Thus, the invention provides CFXTEN compositions in which the degree of activity, bioavailability, half-life or physicochemical characteristic of the fusion protein can be tailored by the selection and placement of the type and length of the XTEN in the CFXTEN compositions. Accordingly, the invention contemplates compositions in which a FVIII from Table 1 or Table 31 and XTEN or XTEN fragment from any one of Tables 3, 4, or 9-13 are produced, for example, in a configuration selected from any one of formulae I-VIII such that the construct has the desired property.

The invention provides methods to produce the CFXTEN compositions that can maintain the FVIII component at therapeutic levels in a subject in need thereof for at least a two-fold, or at least a three-fold, or at least a four-fold, or at least a five-fold greater period of time compared to comparable dosages of the corresponding FVIII not linked to XTEN. In one embodiment of the method, the subject is receiving routine prophylaxis to prevent bleeding episodes. In another embodiment of the method, the subject is receiving treatment for a bleeding episode. In another embodiment of the method, the subject is receiving treatment to raise the circulating blood concentration of procoagulant FVIII above 1%, or above 1-5%, or above 5-40% relative to FVIII concentrations in normal plasma. “Procoagulant” as used herein has its general meaning in the art and generally refers to an activity that promotes clot formation, either in an in vitro assay or in vivo. The method to produce the compositions that can maintain the FVIII component at therapeutic levels includes the steps of selecting one or more XTEN appropriate for conjugation to a FVIII to provide the desired pharmacokinetic properties in view of a given dose and dose regimen, creating a gene construct that encodes the CFXTEN in one of the configurations disclosed herein, transforming an appropriate host cell with an expression vector comprising the encoding gene, expressing the fusion protein under suitable culture conditions, recovering the CFXTEN, administration of the CFXTEN to a mammal followed by assays to verify the pharmacokinetic properties and the activity of the CFXTEN fusion protein (e.g., the ability to maintain hemostasis or serve as a procoagulant) and the safety of the administered composition. Those compositions exhibiting the desired properties are selected for further use. CFXTEN created by the methods provided herein can result in increased efficacy of the administered composition by, amongst other properties, maintaining the circulating concentrations of the procoagulant FVIII component at therapeutic levels for an enhanced period of time.

The invention provides methods to assay the CFXTEN fusion proteins of differing composition or configuration in order to provide CFXTEN with the desired degree of procoagulant and therapeutic activity and pharmacokinetic properties, as well as a sufficient safety profile. Specific in vivo and ex vivo biological assays are used to assess the activity and functional characteristics of each configured CFXTEN and/or FVIII component to be incorporated into CFXTEN, including but not limited to the assays of the Examples, those assays of Table 27, as well as the following assays or other such assays known in the art for assaying the properties and effects of FVIII. Functional assays can be conducted that allow determination of coagulation activity, such as one-stage clotting assay and two-stage clotting assay (Barrowcliffe T W, Semin Thromb Hemost. (2002) 28(3):247-256), activated partial prothrombin (aPTT) assays (Belaaouaj A A et al., J. Biol. Chem. (2000) 275:27123-8; Diaz-Collier J A. Haemost (1994) 71:339-46), chromogenic FVIII assays (Lethagen, S., et al., Scandinavian J Haematology (1986) 37:448-453), or animal model pharmacodynamic assays including bleeding time or thrombelastography (TEG or ROTEM), among others. Other assays include determining the binding affinity of a CFXTEN for the target substrate using binding or competitive binding assays, such as Biacore assays with chip-bound receptors or binding proteins or ELISA assays, as described in U.S. Pat. No. 5,534,617, assays described in the Examples herein, radio-receptor assays, or other assays known in the art. The foregoing assays can also be used to assess FVIII sequence variants (assayed as single components or as CFXTEN fusion proteins) and can be compared to the native FVIII to determine whether they have the same degree of procoagulant activity as the native CF, or some fraction thereof such that they are suitable for inclusion in CFXTEN; e.g., at least about 30%, or at least about 40%, or at least about 50%, or at least about 60%, or at least about 70%, or at least about 80%, or at least about 90% of the activity compared to the native FVIII.

Dose optimization is important for all drugs. A therapeutically effective dose or amount of the CFXTEN varies according to factors such as the disease state, age, sex, and weight of the individual, and the ability of the administered fusion protein to elicit a desired response in the individual. For example, a standardized single dose of FVIII for all patients presenting with diverse bleeding conditions or abnormal clinical parameters (e.g., neutralizing antibodies) may not always be effective. A consideration of these factors is well within the purview of the ordinarily skilled clinician for the purpose of determining the therapeutically or pharmacologically effective amount of the CFXTEN and the appropriated dosing schedule, versus that amount that would result in insufficient potency such that clinical improvement is not achieved.

The invention provides methods to establish a dose regimen for the CFXTEN pharmaceutical compositions of the invention. The methods include administration of consecutive doses of a therapeutically effective amount of the CFXTEN pharmaceutical composition using variable periods of time between doses to determine that interval of dosing sufficient to achieve and/or maintain the desired parameter, blood level or clinical effect; such consecutive doses of a therapeutically effective amount at the effective interval establishes the therapeutically effective dose regimen for the CFXTEN for a factor VIII-related disease state or condition. A prophylactically effective amount refers to an amount of CFXTEN required for the period of time necessary to prevent a physiologic or clinical result or event; e.g., delayed onset of a bleeding episode or maintaining blood concentrations of procoagulant FVIII or equivalent above a threshold level (e.g., 1-5% to 5-40% of normal). In the methods of treatment, the dosage amount of the CFXTEN that is administered to a subject ranges from about 5 to 300 IU/kg/dose, or from about 10 to 100 IU/kg/dose, or from about 20 to about 65 IU/kg/dose, or from about 20 to about 40 IU/kg/dose for a subject. A suitable dosage may also depend on other factors that may influence the response to the drug; e.g., bleeding episodes generally requiring higher doses at more frequent intervals compared to prophylaxis.

In some embodiments, the method comprises administering a therapeutically-effective amount of a pharmaceutical composition comprising a CFXTEN fusion protein composition comprising FVIII linked to one or more XTEN sequences and at least one pharmaceutically acceptable carrier to a subject in need thereof, wherein the administration results in a greater improvement in at least one of the disclosed parameters or physiologic conditions, or results in a more favorable clinical outcome mediated by the FVIII component of the CFXTEN compared to the effect on the parameter, condition or clinical outcome mediated by administration of a pharmaceutical composition comprising a FVIII not linked to XTEN and administered at a comparable dose. In one embodiment of the foregoing, the improvement is achieved by administration of the CFXTEN pharmaceutical composition at a dose that achieves a circulating concentration of procoagulant FVIII (or equivalent) above a threshold level (e.g., 1-5% to 5-40% of normal), thereby establishing the therapeutically effective dose. In another embodiment of the foregoing, the improvement is achieved by administration of multiple consecutive doses of the CFXTEN pharmaceutical composition using a therapeutically effective dose regimen that maintains a circulating concentration of procoagulant FVIII (or equivalent) above a threshold level (e.g., 1-5% to 5-40% of normal) for the length of the dosing period.

In many cases, the therapeutic levels for FVIII in subjects of different ages or degree of disease have been established and are available in published literature or are stated on the drug label for approved products containing the FVIII. For example, the Subcommittee on Factor VIII and Factor IX of the Scientific and Standardization Committee of the International Society on Thrombosis and Haemostasis posted, on the ISTH Website 29 Nov. 2000, that the most widely used measure of hemophilia A is established by determining the circulating concentrations of plasma FVIII procoagulant levels, with persons with <1% (<0.01 IU/ml) factor VIII defined as severe; 1-5% (0.01-0.05 IU/ml) as moderately severe; and >5-40% (0.05-<0.40 IU/ml) as mild, where normal is 1 IU/ml of factor VIIIC (100%). The therapeutic levels can be established for new compositions, including those CFXTEN and pharmaceutical compositions comprising CFXTEN of the disclosure, using standard methods. The methods for establishing the therapeutic levels and dosing schedules for a given composition are known to those of skill in the art (see, e.g., Goodman & Gilman's The Pharmacological Basis of Therapeutics, 11^{th} Edition, McGraw-Hill (2005)). For example, by using dose-escalation studies in subjects with the target disease or disorder to determine efficacy or a desirable pharmacologic effect, appearance of adverse events, and determination of circulating blood levels, the therapeutic blood levels for a given subject or population of subjects can be determined for a given drug or biologic. The dose escalation studies would evaluate the activity of a CFXTEN through studies in a subject or group of hemophilia A subjects. The studies would monitor blood levels of procoagulant, as well as physiological or clinical parameters as known in the art or as described herein for one or more parameters associated with the factor VIII-related disease or disorder, or clinical parameters associated with a beneficial outcome, together with observations and/or measured parameters to determine the no effect dose, adverse events, minimum effective dose and the like, together with measurement of pharmacokinetic parameters that establish the determined or derived circulating blood levels. The results can then be correlated with the dose administered and the blood concentrations of the therapeutic that are coincident with the foregoing determined parameters or effect levels. By these methods, a range of doses and blood concentrations can be correlated to the minimum effective dose as well as the maximum dose and blood concentration at which a desired effect occurs and the period for which it can be maintained, thereby establishing the therapeutic blood levels and dosing schedule for the composition. Thus, by the foregoing methods, a Cₘᵢₙ blood level is established, below which the CFXTEN fusion protein would not have the desired pharmacologic effect and a Cₘₐₓ blood level, above which side effects such as thrombosis may occur (Brobrow, R S, JABFP (2005) 18(2):147-149), establishing the therapeutic window for the composition.

One of skill in the art can, by the means disclosed herein or by other methods known in the art, confirm that the administered CFXTEN remains at therapeutic blood levels to maintain hemostasis for the desired interval or requires adjustment in dose or length or sequence of XTEN. Further, the determination of the appropriate dose and dose frequency to keep the CFXTEN within the therapeutic window establishes the therapeutically effective dose regimen; the schedule for administration of multiple consecutive doses using a therapeutically effective dose of the fusion protein to a subject in need thereof resulting in consecutive Cₘₐₓ peaks and/or Cₘᵢₙ troughs that remain above therapeutically-effective concentrations and result in an improvement in at least one measured parameter relevant for the target disease, disorder or condition. In one embodiment, the CFXTEN or a pharmaceutical compositions comprising CFXTEN administered at an appropriate dose to a subject results in blood concentrations of the CFXTEN fusion protein that remains above the minimum effective concentration to maintain hemostasis for a period at least about two-fold longer compared to the corresponding FVIII not linked to XTEN and administered at a comparable dose; alternatively at least about three-fold longer; alternatively at least about four-fold longer; alternatively at least about five-fold longer; alternatively at least about six-fold longer; alternatively at least about seven-fold longer; alternatively at least about eight-fold longer; alternatively at least about nine-fold longer, alternatively at least about ten-fold longer, or at least about twenty-fold longer or greater compared to the corresponding FVIII not linked to XTEN and administered at a comparable dose. As used herein, an “appropriate dose” means a dose of a drug or biologic that, when administered to a subject, would result in a desirable therapeutic or pharmacologic effect and/or a blood concentration within the therapeutic window.

In one embodiment, the CFXTEN or a pharmaceutical compositions comprising CFXTEN administered at a therapeutically effective dose regimen results in a gain in time of at least about three-fold longer; alternatively at least about four-fold longer; alternatively at least about five-fold longer; alternatively at least about six-fold longer; alternatively at least about seven-fold longer; alternatively at least about eight-fold longer; alternatively at least about nine-fold longer or at least about ten-fold longer between at least two consecutive Cₘₐₓ peaks and/or Cₘᵢₙ troughs for blood levels of the fusion protein compared to the corresponding biologically active protein of the fusion protein not linked to the XTEN and administered at a comparable dose regimen to a subject. In another embodiment, the CFXTEN administered at a therapeutically effective dose regimen results in a comparable improvement in one, or two, or three or more measured parameters using less frequent dosing or a lower total dosage in IUs of the fusion protein of the pharmaceutical composition compared to the corresponding biologically active protein component(s) not linked to the XTEN and administered to a subject using a therapeutically effective dose regimen for the FVIII. The measured parameters include any of the clinical, biochemical, or physiological parameters disclosed herein, or others known in the art for assessing subjects with factor VIII-related disorders.

(b) Pharmacology and Pharmaceutical Properties of CFXTEN

The present invention provides CFXTEN compositions comprising FVIII covalently linked to XTEN that have enhanced pharmaceutical and pharmacology properties compared to FVIII not linked to XTEN, as well as methods to enhance the therapeutic and/or procoagulant effect of the FVIII components of the compositions. In addition, the invention provides CFXTEN compositions with enhanced properties compared to those art-known fusion proteins of factor VIII containing albumin, immunoglobulin polypeptide partners, polypeptides of shorter length and/or polypeptide partners with repetitive sequences. In addition, CFXTEN fusion proteins provide significant advantages over chemical conjugates, such as pegylated constructs of FVIII, notably the fact that recombinant CFXTEN fusion proteins can be made in host cell expression systems, which can reduce time and cost at both the research and development and manufacturing stages of a product, as well as result in a more homogeneous, defined product with less toxicity from both the product and metabolites of the CFXTEN compared to pegylated conjugates.

As therapeutic agents, the CFXTEN possesses a number of advantages over therapeutics not comprising XTEN, including one or more of the following non-limiting properties: increased solubility, increased thermal stability, reduced immunogenicity, increased apparent molecular weight, reduced renal clearance, reduced proteolysis, reduced metabolism, enhanced therapeutic efficiency, less frequent dosage regimen with increased time between doses capable of maintaining hemostasis in a subject with hemophilia A, the ability to administer the CFXTEN composition subcutaneously or intramuscularly, a “tailored” rate of absorption when administered subcutaneously or intramuscularly, enhanced lyophilization stability, enhanced serum/plasma stability, increased terminal half-life, increased solubility in blood stream, decreased binding by neutralizing antibodies, decreased active clearance, tailored substrate binding affinity, stability to degradation, stability to freeze-thaw, stability to proteases, stability to ubiquitination, ease of administration, compatibility with other pharmaceutical excipients or carriers, persistence in the subject, increased stability in storage (e.g., increased shelf-life), and the like. The net effect of the enhanced properties is that the use of a CFXTEN composition can result in an overall enhanced therapeutic effect compared to a FVIII not linked to XTEN, result in economic benefits associated with less frequent dosing, and/or result in improved patient compliance when administered to a subject with a factor VIII-related disease, disorder or condition.

In one embodiment, XTEN as a fusion partner increases the solubility of the FVIII payload. Accordingly, where enhancement of the pharmaceutical or physicochemical properties of the FVIII is desirable, such as the degree of aqueous solubility or stability, the length and/or the motif family composition of the XTEN sequences incorporated into the fusion protein may each be selected to confer a different degree of solubility and/or stability on the respective fusion proteins such that the overall pharmaceutical properties of the CFXTEN composition are enhanced. The CFXTEN fusion proteins can be constructed and assayed, using methods described herein, to confirm the physicochemical properties and the choice of the XTEN length sequence or location adjusted, as needed, to result in the desired properties. In one embodiment, the CFXTEN has an aqueous solubility that is at least about 25% greater compared to a FVIII not linked to the XTEN, or at least about 30%, or at least about 40%, or at least about 50%, or at least about 75%, or at least about 100%, or at least about 200%, or at least about 300%, or at least about 400%, or at least about 500%, or at least about 1000% greater than the corresponding FVIII not linked to XTEN.

The invention provides methods to produce and recover expressed CFXTEN from a host cell with enhanced solubility and ease of recovery compared to FVIII not linked to XTEN. In one embodiment, the method includes the steps of transforming a eukaryotic host cell with a polynucleotide encoding a CFXTEN with one or more XTEN components of cumulative sequence length greater than about 100, or greater than about 200, or greater than about 400, or greater than about 600, or greater than about 800, or greater than about 1000, or greater than about 2000, or greater than about 3000 amino acid residues, expressing the CFXTEN fusion protein in the host cell under suitable culture and induction conditions, and recovering the expressed fusion protein in soluble form. In one embodiment, the one or more XTEN of the CFXTEN fusion proteins each have at least about 80% sequence identity, or about 90%, or about 91%, or about 92%, or about 93%, or about 94%, or about 95%, or about 96%, or about 97%, or about 98%, or about 99%, to about 100% sequence identity compared to one or more XTEN selected from any one of Tables 4, and 9-13, or fragments thereof, and the FVIII have at least about 80% sequence identity, or about 90%, or about 91%, or about 92%, or about 93%, or about 94%, or about 95%, or about 96%, or about 97%, or about 98%, or about 99%, or 100% sequence identity compared to a FVIII selected from Table 1, and the CFXTEN components are in an N— to C-terminus configuration selected from any one of the configuration embodiments disclosed herein.

### VI). Uses of the CFXTEN Compositions

In another aspect, the invention provides a method for achieving a beneficial effect in bleeding disorders and/or in a factor VIII-related disease, disorder or condition mediated by FVIII. As used herein, “factor VIII-related diseases, disorders or conditions” is intended to include, but is not limited to factor VIII deficiencies, bleeding disorders related to factor VIII deficiency, hemophilia A, and bleeding from trauma or surgery or vascular injury that can be ameliorated or corrected by administration of FVIII to a subject. The present invention provides methods for treating a subject, such as a human, with a factor VIII-related disease, disorder or condition in order to achieve a beneficial effect, addressing disadvantages and/or limitations of other methods of treatment using factor VIII preparations that have a relatively short terminal half-life, require repeated administrations, or have unfavorable pharmacoeconomics.

Hemostasis is regulated by multiple protein factors, and such proteins, as well as analogues thereof, have found utility in the treatment of factor VIII-related diseases, disorders and conditions. However, the use of commercially-available FVIII has met with less than optimal success in the management of subjects afflicted with such diseases, disorders and conditions. In particular, dose optimization and frequency of dosing is important for FVIII used in maintaining circulating FVIII concentrations above threshold levels needed for hemostasis, as well as the treatment or prevention of bleeding episodes in hemophilia A subjects. The fact that FVIII products have a short half-life necessitates frequent dosing in order to achieve clinical benefit, which results in difficulties in the management of such patients.

As established by the Subcommittee on Factor VIII and Factor IX of the Scientific and Standardization Committee of the International Society on Thrombosis and Haemostasis (posted on the ISTH Website 29 Nov. 2000), the most widely used measure of the severity of hemophilia A is established by determining the circulating concentrations of plasma FVIII procoagulant levels, with persons with <1% (<0.01 IU/ml) factor VIII defined as severe; 1-5% (0.01-0.05 IU/ml) as moderately severe; and >5-40% (0.05-<0.40 IU/ml) as mild, where normal is 1 IU/ml of factor VIIIC (100%).

In some embodiments, the invention provides methods of treatment comprising administering a therapeutically- or prophylactically-effective amount of a CFXTEN pharmaceutical composition to a subject suffering from or at risk of developing a factor VIII-related disease, disorder or condition, wherein the administration results in the improvement of one or more biochemical, physiological or clinical parameters associated with the disease, disorder or condition. In one embodiment of the foregoing method, the administered CFXTEN comprises a FVIII with at least about 80%, or at least about 90%, or at least about 95%, or at least about 97%, or at least about 99% sequence identity to a factor VIII of Table 1. In another embodiment of the foregoing method, the administered CFXTEN comprises a FVIII with at least about 80%, or at least about 90%, or at least about 95%, or at least about 97%, or at least about 99% sequence identity to a factor VIII of Table 1 or Table 31 and at least one XTEN sequence with at least about 80%, or at least about 90%, or at least about 95%, or at least about 97%, or at least about 99% sequence identity to an XTEN of Table 4. In another embodiment of the foregoing method, the administered CFXTEN has a sequence with at least about 80%, or at least about 90%, or at least about 95%, or at least about 97%, or at least about 99% sequence identity to a sequence of Table 14, Table 28, Table 29, or Table 30.

The invention provides methods of treatment comprising administering a therapeutically-effective amount of an CFXTEN composition to a subject suffering from hemophilia A wherein the administration results in the improvement of one or more biochemical, physiological or clinical parameters associated with the FVIII disease, disorder or condition for a period at least two-fold longer, or at least four-fold longer, or at least five-fold longer, or at least six-fold longer compared to a FVIII not linked to XTEN and administered at a comparable dose. In one embodiment of the method of treatment, a CFXTEN composition or a pharmaceutical compositions comprising CFXTEN is administered to a subject suffering from hemophilia A in an amount sufficient to increase the circulating FVIII procoagulant concentration to greater than 0.01 IU/ml (1% of normal), or greater than 0.01-0.05 IU/ml (1%-5% of normal), or greater than >0.05-<0.40 IU/ml (>5%-<40% of normal). In the foregoing embodiment, the specified concentration is maintained for at least about 12 h, or at least about 24 h, or at least about 48 h, or at least about 72 h, or at least about 96 h, or at least about 120 h, or at least about 144 h, or at least about 168 h, or greater. In another embodiment of the method of treatment, a CFXTEN fusion protein or a pharmaceutical compositions comprising CFXTEN is administered to a subject with anti-FVIII antibodies in an amount sufficient to increase the active, circulating FVIII procoagulant concentration to greater than 0.01 IU/ml (0.01-0.05 IU/ml (1% of normal), or greater than 0.01-0.05 IU/ml (1%-5% of normal), or greater than >0.05-<0.40 IU/ml (>5%-<40% of normal). In the foregoing embodiment, the specified concentration is maintained for at least about 12 h, or at least about 24 h, or at least about 48 h, or at least about 72 h, or at least about 96 h, or at least about 120 h, or at least about 144 h, or at least about 168 h, or greater. In another embodiment of the method of treatment, a therapeutically effective amount of a CFXTEN composition or a pharmaceutical compositions comprising CFXTEN is administered to a subject suffering from a bleeding episode, wherein the administration results in the resolution of the bleeding for a duration at least two-fold, or at least three-fold, or at least four-fold longer compared to a FVIII not linked to XTEN and administered to a subject at a comparable dose. In another embodiment, the administration of a therapeutically effective amount of a CFXTEN composition or a pharmaceutical compositions comprising CFXTEN to a subject in need thereof results in a greater reduction in a one-stage clotting assay time of at least about 5%, or about 10%, or about 20%, or about 30%, or about 40%, or about 50%, or about 60%, or about 70%, or more in the subject at 2-7 days after the administration compared to the assay time in a subject after administration of a comparable amount of the corresponding FVIII not linked to XTEN. In another embodiment, the administration of a therapeutically effective amount of a CFXTEN or a pharmaceutical compositions comprising CFXTEN to a subject in need thereof results in a reduction in the activated partial prothrombin time of at least about 5%, or about 10%, or about 20%, or about 30%, or about 40%, or about 50%, or about 60%, or about 70%, or more in the subject 2-7 days after administration compared to the activated partial prothrombin time in a subject after administration of a comparable amount of the corresponding FVIII not linked to XTEN. In another embodiment, the administration of a CFXTEN or a pharmaceutical compositions comprising CFXTEN to a subject in need thereof using a therapeutically effective amount results in maintenance of activated partial prothrombin times within 30% of normal in the subject for a period of time that is at least two-fold, or at least about three-fold, or at least about four-fold longer compared to that of a FVIII not linked to XTEN and administered to a subject using a comparable dose.

In some embodiments of the method of treatment, (i) a smaller IU amount of about two-fold less, or about three-fold less, or about four-fold less, or about five-fold less, or about six-fold less, or about eight-fold less, or about 10-fold less of the CFXTEN fusion protein or a pharmaceutical compositions comprising CFXTEN is administered to a subject in need thereof in comparison to the corresponding coagulation factor not linked to the XTEN under an otherwise same dose regimen, and the fusion protein achieves a comparable area under the curve (based on IU/ml) and/or a comparable therapeutic effect as the corresponding FVIII not linked to the XTEN; (ii) the CFXTEN fusion protein is administered less frequently (e.g., every three days, about every seven days, about every 10 days, about every 14 days, about every 21 days, or about monthly) in comparison to the corresponding FVIII not linked to the XTEN under an otherwise same dose amount, and the fusion protein achieves a comparable area under the curve and/or a comparable therapeutic effect as the corresponding coagulation factor not linked to the XTEN; or (iii) an accumulative smaller IU amount of at least about 20%, or about 30%, or about 40%, or about 50%, or about 60%, or about 70%, or about 80%, or about 90% less of the fusion protein is administered in comparison to the corresponding FVIII not linked to the XTEN under an otherwise same dose regimen and the CFXTEN fusion protein achieves a comparable area under the curve and/or a comparable therapeutic effect as the corresponding FVIII not linked to the XTEN. The accumulative smaller IU amount is measured for a period of at least about one week, or about 14 days, or about 21 days, or about one month. In the foregoing embodiments of the method of treatment, the therapeutic effect can be determined by any of the measured parameters described herein, including but not limited to blood concentrations of FVIII, results of an activated partial prothrombin (aPTT) assay, results of a one-stage or two-stage clotting assays, delayed onset of a bleeding episode, results of a chromogenic FVIII assay, or other assays known in the art for assessing coagulopathies of FVIII.

The invention further contemplates that the CFXTEN used in accordance with the methods provided herein can be administered in conjunction with other treatment methods and compositions (e.g., other coagulation proteins) useful for treating factor VIII-related diseases, disorders, and conditions, or conditions for which coagulation factor is adjunctive therapy; e.g., bleeding episodes due to injury or surgery.

In another aspect, the invention provides a method of preparing a medicament for treatment of a factor VIII-related disease, disorder or condition, comprising combining a factor VIII sequence selected from Table 1 or Table 31 with one or more XTEN to result in a CFXTEN fusion protein, wherein the CFXTEN retains at least a portion of the activity of the native FVIII, and further combining the CFXTEN with at least one pharmaceutically acceptable carrier, resulting in a CFXTEN pharmaceutical composition. In one embodiment of the method, the factor VIII has a sequence with at least about 80%, or at least about 90%, or at least about 95%, or at least about 97%, or at least about 99% sequence identity compared to a sequence selected from Table 1 or Table 31 and the one or more XTEN has a sequence with at least about 80%, or at least about 90%, or at least about 95%, or at least about 97%, or at least about 99% sequence identity compared to a sequence selected from any one of Tables 3, 4, and 9-13, or a fragment thereof. In another embodiment of the method, the CFXTEN has a sequence with at least about 80%, or at least about 90%, or at least about 95%, or at least about 97%, or at least about 99% sequence identity compared to a sequence selected from any one of Tables 14 and 28-30.

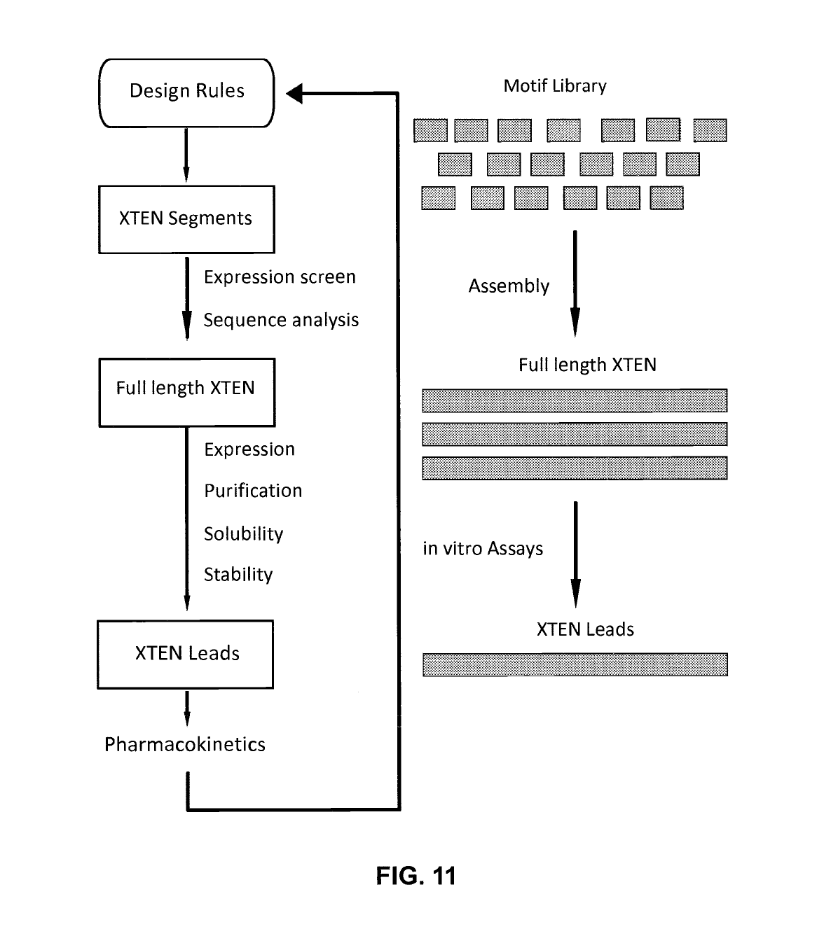

In another aspect, the invention provides a method of designing the CFXTEN compositions to achieve desired pharmacokinetic, pharmacologic or pharmaceutical properties. In general, the steps in the design and production of the fusion proteins and the inventive compositions, as illustrated in FIGS. 11-13, include: (1) the selection of a FVIII (e.g., native proteins, sequences of Table 1, analogs or derivatives with activity) to treat the particular disease, disorder or condition; (2) selecting the XTEN that will confer the desired PK and physicochemical characteristics on the resulting CFXTEN (e.g., the administration of the CFXTEN composition to a subject results in the fusion protein being maintained within the therapeutic window for a greater period compared to FVIII not linked to XTEN); (3) establishing a desired N- to C-terminus configuration of the CFXTEN to achieve the desired efficacy or PK parameters; (4) establishing the design of the expression vector encoding the configured CFXTEN; (5) transforming a suitable host with the expression vector; and (6) expression and recovery of the resultant fusion protein. For those CFXTEN for which an increase in half-life (greater than 24 h) or an increased period of time spent above the minimum effective concentration is desired, the XTEN chosen for incorporation generally has at least about 288, or about 432, or about 576, or about 864, or about 875, or about 912, or about 923 amino acid residues where a single XTEN is to be incorporated into the CFXTEN. In another embodiment, the CFXTEN comprises a first XTEN of the foregoing lengths, and at least a second XTEN of about 36, or about 72, or about 144, or about 288, or about 576, or about 864, or about 875, or about 912, or about 923, or about 1000 or more amino acid residues. The location of the XTEN within the fusion protein can include one, two, three, four, five or more locations selected from Table 5, Table 25, or FIG. 7.

In other embodiments, where an increase in a pharmaceutical property (e.g., solubility) is desired, a CFXTEN is designed to include multiple XTEN of shorter lengths. In one embodiment of the foregoing, the CFXTEN comprises a FVIII linked to multiple XTEN having at least about 24, or about 36, or about 48, or about 60, or about 72, or about 84, or about 96 amino acid residues inserted at sites selected from Table 5, Table 25, or FIG. 7, in which the solubility of the fusion protein under physiologic conditions is at least three-fold greater than the corresponding FVIII not linked to XTEN, or alternatively, at least four-fold, or five-fold, or six-fold, or seven-fold, or eight-fold, or nine-fold, or at least 10-fold, or at least 20-fold, or at least 30-fold, or at least 50-fold, or at least 60-fold or greater than FVIII not linked to XTEN. In one embodiment of the foregoing, the CF is a FVIII with at least about 80%, or about 90%, or about 95% identity to a sequence from Table 1 or Table 31 and the XTEN is a sequence with at least about 80%, or about 90%, or about 95% sequence identity compared to a sequence from any one of Tables 3, 4, and 9-13.

In another aspect, the invention provides methods of making CFXTEN compositions to improve ease of manufacture, result in increased stability, increased water solubility, and/or ease of formulation, as compared to the native FVIII. In one embodiment, the invention includes a method of increasing the water solubility of a FVIII comprising the step of linking the FVIII to one or more XTEN such that a higher concentration in soluble form of the resulting CFXTEN can be achieved, under physiologic conditions, compared to the FVIII in an un-fused state. Factors that contribute to the property of XTEN to confer increased water solubility of CFs when incorporated into a fusion protein include the high solubility of the XTEN fusion partner and the low degree of self-aggregation between molecules of XTEN in solution. In some embodiments, the method results in a CFXTEN fusion protein wherein the water solubility is at least about 20%, or at least about 30% greater, or at least about 50% greater, or at least about 75% greater, or at least about 90% greater, or at least about 100% greater, or at least about 150% greater, or at least about 200% greater, or at least about 400% greater, or at least about 600% greater, or at least about 800% greater, or at least about 1000% greater, or at least about 2000% greater under physiologic conditions, compared to the un-fused FVIII. In one embodiment, the XTEN of the CFXTEN fusion protein is a sequence with at least about 80%, or about 90%, or about 95% sequence identity compared to a sequence from any one of Tables 3, 4, and 9-13.

In another embodiment, the invention includes a method of increasing the shelf-life of a FVIII comprising the step of linking the FVIII with one or more XTEN selected such that the shelf-life of the resulting CFXTEN is extended compared to the FVIII in an un-fused state. As used herein, shelf-life refers to the period of time over which the functional activity of a FVIII or CFXTEN that is in solution or in some other storage formulation remains stable without undue loss of activity. As used herein, “functional activity” refers to a pharmacologic effect or biological activity, such as the ability to bind a receptor or ligand, or substrate, or to display procoagulant activity associated with FVIII, as known in the art. A FVIII that degrades or aggregates generally has reduced functional activity or reduced bioavailability compared to one that remains in solution. Factors that contribute to the ability of the method to extend the shelf life of CFs when incorporated into a fusion protein include increased water solubility, reduced self-aggregation in solution, and increased heat stability of the XTEN fusion partner. In particular, the low tendency of XTEN to aggregate facilitates methods of formulating pharmaceutical preparations containing higher drug concentrations of CFs, and the heat-stability of XTEN contributes to the property of CFXTEN fusion proteins to remain soluble and functionally active for extended periods. In one embodiment, the method results in CFXTEN fusion proteins with “prolonged” or “extended” shelf-life that exhibit greater activity relative to a standard that has been subjected to the same storage and handling conditions. The standard may be the un-fused full-length FVIII. In one embodiment, the method includes the step of formulating the isolated CFXTEN with one or more pharmaceutically acceptable excipients that enhance the ability of the XTEN to retain its unstructured conformation and for the CFXTEN to remain soluble in the formulation for a time that is greater than that of the corresponding un-fused FVIII. In one embodiment, the method comprises linking a FVIII to one or more XTEN selected from any one of Tables 3, 4, and 9-13 to create a CFXTEN fusion protein results in a solution that retains greater than about 100% of the functional activity, or greater than about 105%, 110%, 120%, 130%, 150% or 200% of the functional activity of a standard when compared at a given time point and when subjected to the same storage and handling conditions as the standard, thereby increasing its shelf-life.

Shelf-life may also be assessed in terms of functional activity remaining after storage, normalized to functional activity when storage began. CFXTEN fusion proteins of the invention with prolonged or extended shelf-life as exhibited by prolonged or extended functional activity retain about 50% more functional activity, or about 60%, 70%, 80%, or 90% more of the functional activity of the equivalent FVIII not linked to XTEN when subjected to the same conditions for the same period of time. For example, a CFXTEN fusion protein of the invention comprising coagulation factor fused to one or more XTEN sequences selected from any one of Tables 3, 4, and 9-13 retains about 80% or more of its original activity in solution for periods of up to 2 weeks, or 4 weeks, or 6 weeks or longer under various temperature conditions. In some embodiments, the CFXTEN retains at least about 50%, or about 60%, or at least about 70%, or at least about 80%, and most preferably at least about 90% or more of its original activity in solution when heated at 80° C. for 10 min. In other embodiments, the CFXTEN retains at least about 50%, preferably at least about 60%, or at least about 70%, or at least about 80%, or alternatively at least about 90% or more of its original activity in solution when heated or maintained at 37° C. for about 7 days. In another embodiment, CFXTEN fusion protein retains at least about 80% or more of its functional activity after exposure to a temperature of about 30° C. to about 70° C. over a period of time of about one hour to about 18 hours. In the foregoing embodiments hereinabove described in this paragraph, the retained activity of the CFXTEN is at least about two-fold, or at least about three-fold, or at least about four-fold, or at least about five-fold, or at least about six-fold greater at a given time point than that of the corresponding FVIII not linked to the XTEN.

### VII). The Nucleic Acids Sequences of the Invention

The present invention provides isolated polynucleic acids encoding CFXTEN chimeric fusion proteins and sequences complementary to polynucleic acid molecules encoding CFXTEN chimeric fusion proteins, including homologous variants thereof. In another aspect, the invention encompasses methods to produce polynucleic acids encoding CFXTEN chimeric fusion proteins and sequences complementary to polynucleic acid molecules encoding CFXTEN chimeric fusion protein, including homologous variants thereof. In general, and as illustrated in FIGS. 11-13, the methods of producing a polynucleotide sequence coding for a CFXTEN fusion protein and expressing the resulting gene product include assembling nucleotides encoding FVIII and XTEN, ligating the components in frame, incorporating the encoding gene into an expression vector appropriate for a host cell, transforming the appropriate host cell with the expression vector, and culturing the host cell under conditions causing or permitting the fusion protein to be expressed in the transformed host cell, thereby producing the biologically-active CFXTEN polypeptide, which is recovered as an isolated fusion protein by standard protein purification methods known in the art. Standard recombinant techniques in molecular biology is used to make the polynucleotides and expression vectors of the present invention.

In accordance with the invention, nucleic acid sequences that encode CFXTEN (or its complement) is used to generate recombinant DNA molecules that direct the expression of CFXTEN fusion proteins in appropriate host cells. Several cloning strategies are suitable for performing the present invention, many of which is used to generate a construct that comprises a gene coding for a fusion protein of the CFXTEN composition of the present invention, or its complement. In some embodiments, the cloning strategy is used to create a gene that encodes a monomeric CFXTEN that comprises at least a first FVIII and at least a first XTEN polypeptide, or their complement. In one embodiment of the foregoing, the gene comprises a sequence encoding a FVIII or sequence variant. In other embodiments, the cloning strategy is used to create a gene that encodes a monomeric CFXTEN that comprises nucleotides encoding at least a first molecule of FVIII or its complement and a first and at least a second XTEN or their complement that is used to transform a host cell for expression of the fusion protein of the CFXTEN composition. In the foregoing embodiments hereinabove described in this paragraph, the genes can further comprise nucleotides encoding spacer sequences that also encode cleavage sequence(s).

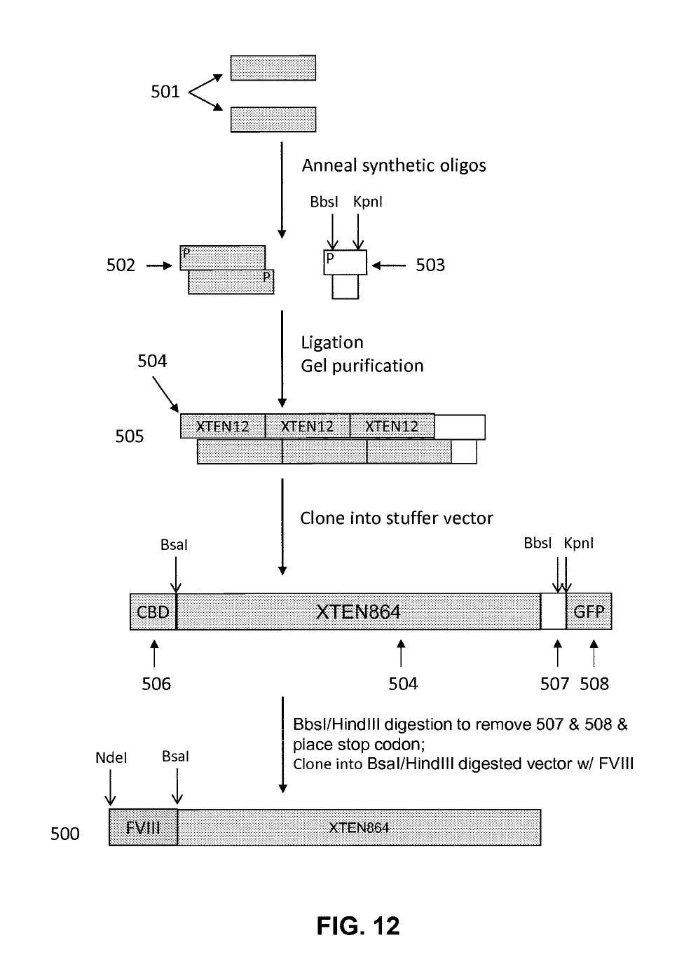

In designing a desired XTEN sequences, it was discovered that the non-repetitive nature of the XTEN of the inventive compositions is achieved despite use of a “building block” molecular approach in the creation of the XTEN-encoding sequences. This was achieved by the use of a library of polynucleotides encoding peptide sequence motifs, described above, that are then ligated and/or multimerized to create the genes encoding the XTEN sequences (see FIGS. 11 and 12 and Examples). Thus, while the XTEN(s) of the expressed fusion protein may consist of multiple units of as few as four different sequence motifs, because the motifs themselves consist of non-repetitive amino acid sequences, the overall XTEN sequence is rendered non-repetitive. Accordingly, in one embodiment, the XTEN-encoding polynucleotides comprise multiple polynucleotides that encode non-repetitive sequences, or motifs, operably linked in frame and in which the resulting expressed XTEN amino acid sequences are non-repetitive.

In one approach, a construct is first prepared containing the DNA sequence corresponding to CFXTEN fusion protein. DNA encoding the FVIII of the compositions is obtained from a cDNA library prepared using standard methods from tissue or isolated cells believed to possess FVIII mRNA and to express it at a detectable level. Libraries are screened with probes containing, for example, about 20 to 100 bases designed to identify the FVIII gene of interest by hybridization using conventional molecular biology techniques. The best candidates for probes are those that represent sequences that are highly homologous for coagulation factor, and should be of sufficient length and sufficiently unambiguous that false positives are minimized, but may be degenerate at one or more positions. If necessary, the coding sequence can be obtained using conventional primer extension procedures as described in Sambrook, et al., supra, to detect precursors and processing intermediates of mRNA that may not have been reverse-transcribed into cDNA. One can then use polymerase chain reaction (PCR) methodology to amplify the target DNA or RNA coding sequence to obtain sufficient material for the preparation of the CFXTEN constructs containing the FVIII gene. Assays can then be conducted to confirm that the hybridizing full-length genes are the desired FVIII gene(s). By these conventional methods, DNA can be conveniently obtained from a cDNA library prepared from such sources. The FVIII encoding gene(s) is also be obtained from a genomic library or created by standard synthetic procedures known in the art (e.g., automated nucleic acid synthesis using, for example one of the methods described in Engels et al. (Agnew. Chem. Int. Ed. Engl., 28:716-734 1989)), using DNA sequences obtained from publicly available databases, patents, or literature references. Such procedures are well known in the art and well described in the scientific and patent literature. For example, sequences can be obtained from Chemical Abstracts Services (CAS) Registry Numbers (published by the American Chemical Society) and/or GenBank Accession Numbers (e.g., Locus ID, NP_XXXXX, and XP—XXXXX) Model Protein identifiers available through the National Center for Biotechnology Information (NCBI) webpage, available on the world wide web at ncbi.nlm.nih.gov that correspond to entries in the CAS Registry or GenBank database that contain an amino acid sequence of the protein of interest or of a fragment or variant of the protein. For such sequence identifiers provided herein, the summary pages associated with each of these CAS and GenBank and GenSeq Accession Numbers as well as the cited journal publications (e.g., PubMed ID number (PMID)) are each incorporated by reference in their entireties, particularly with respect to the amino acid sequences described therein. In one embodiment, the FVIII encoding gene encodes a protein from any one of Table 1, or a fragment or variant thereof.

A gene or polynucleotide encoding the FVIII portion of the subject CFXTEN protein, in the case of an expressed fusion protein that comprises a single FVIII is then be cloned into a construct, which is a plasmid or other vector under control of appropriate transcription and translation sequences for high level protein expression in a biological system. In a later step, a second gene or polynucleotide coding for the XTEN is genetically fused to the nucleotides encoding the N- and/or C-terminus of the FVIII gene by cloning it into the construct adjacent and in frame with the gene(s) coding for the FVIII. This second step occurs through a ligation or multimerization step. In the foregoing embodiments hereinabove described in this paragraph, it is to be understood that the gene constructs that are created can alternatively be the complement of the respective genes that encode the respective fusion proteins.

The gene encoding for the XTEN can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate short sequences of polynucleotides encoding XTEN into longer XTEN genes of a desired length and sequence. In one embodiment, the method ligates two or more codon-optimized oligonucleotides encoding XTEN motif or segment sequences of about 9 to 14 amino acids, or about 12 to 20 amino acids, or about 18 to 36 amino acids, or about 48 to about 144 amino acids, or about 144 to about 288 or longer, or any combination of the foregoing ranges of motif or segment lengths.

Alternatively, the disclosed method is used to multimerize XTEN-encoding sequences into longer sequences of a desired length; e.g., a gene encoding 36 amino acids of XTEN can be dimerized into a gene encoding 72 amino acids, then 144, then 288, etc. Even with multimerization, XTEN polypeptides can be constructed such that the XTEN-encoding gene has low or virtually no repetitiveness through design of the codons selected for the motifs of the shortest unit being used, which can reduce recombination and increase stability of the encoding gene in the transformed host.

Genes encoding XTEN with non-repetitive sequences are assembled from oligonucleotides using standard techniques of gene synthesis. The gene design can be performed using algorithms that optimize codon usage and amino acid composition. In one method of the invention, a library of relatively short XTEN-encoding polynucleotide constructs is created and then assembled, as described above. The resulting genes are then assembled with genes encoding FVIII or regions of FVIII, as illustrated in FIGS. 11 and 12, and the resulting genes used to transform a host cell and produce and recover the CFXTEN for evaluation of its properties, as described herein.

In some embodiments, the CFXTEN sequence is designed for optimized expression by inclusion of an N-terminal sequence (NTS) XTEN, rather than using a leader sequence known in the art. In one embodiment, the NTS is created by inclusion of encoding nucleotides in the XTEN gene determined to result in optimized expression when joined to the gene encoding the fusion protein. In one embodiment, the N-terminal XTEN sequence of the expressed CFXTEN is optimized for expression in a eukaryotic cell, such as but not limited to CHO, HEK, COS, yeast, and other cell types know in the art.

### Polynucleotide Libraries

In another aspect, the invention provides libraries of polynucleotides that encode XTEN sequences that are used to assemble genes that encode XTEN of a desired length and sequence.

In certain embodiments, the XTEN-encoding library constructs comprise polynucleotides that encode polypeptide segments of a fixed length. As an initial step, a library of oligonucleotides that encode motifs of 9-14 amino acid residues can be assembled. In a preferred embodiment, libraries of oligonucleotides that encode motifs of 12 amino acids are assembled.

The XTEN-encoding sequence segments can be dimerized or multimerized into longer encoding sequences. Dimerization or multimerization can be performed by ligation, overlap extension, PCR assembly or similar cloning techniques known in the art. This process of can be repeated multiple times until the resulting XTEN-encoding sequences have reached the organization of sequence and desired length, providing the XTEN-encoding genes. As will be appreciated, a library of polynucleotides that encodes, e.g., 12 amino acid motifs can be dimerized and/or ligated into a library of polynucleotides that encode 36 amino acids. Libraries encoding motifs of different lengths; e.g., 9-14 amino acid motifs leading to libraries encoding 27 to 42 amino acids are contemplated by the invention. In turn, the library of polynucleotides that encode 27 to 42 amino acids, and preferably 36 amino acids (as described in the Examples) can be serially dimerized into a library containing successively longer lengths of polynucleotides that encode XTEN sequences of a desired length for incorporation into the gene encoding the CFXTEN fusion protein, as disclosed herein.

A more efficient way to optimize the DNA sequence encoding XTEN is based on combinatorial libraries. The gene encoding XTEN can be designed and synthesized in segment such that multiple codon versions are obtained for each segment. These segments can be randomly assembled into a library of genes such that each library member encodes the same amino acid sequences but library members comprise a large number of codon versions. Such libraries can be screened for genes that result in high-level expression and/or a low abundance of truncation products. The process of combinatorial gene assembly is illustrated in FIG. 16. The genes in FIG. 16 are assembled from 6 base fragments and each fragment is available in 4 different codon versions. This allows for a theoretical diversity of 4096.

In some embodiments, libraries are assembled of polynucleotides that encode amino acids that are limited to specific sequence XTEN families; e.g., AD, AE, AF, AG, AM, or AQ sequences of Table 3. In other embodiments, libraries comprise sequences that encode two or more of the motif family sequences from Table 3. The names and sequences of representative, non-limiting polynucleotide sequences of libraries that encode 36 mers are presented in Tables 9-12, and the methods used to create them are described more fully in the respective Examples. In other embodiments, libraries that encode XTEN are constructed from segments of polynucleotide codons linked in a randomized sequence that encode amino acids wherein at least about 80%, or at least about 90%, or at least about 91%, or at least about 92%, or at least about 93%, or at least about 94%, or at least about 95%, or at least about 97%, or at least about 98%, or at least about 99% of the codons are selected from the group consisting of condons for glycine (G), alanine (A), serine (S), threonine (T), glutamate (E) and proline (P) amino acids. The libraries can be used, in turn, for serial dimerization or ligation to achieve polynucleotide sequence libraries that encode XTEN sequences, for example, of 48, 72, 144, 288, 576, 864, 875, 912, 923, 1318 amino acids, or up to a total length of about 3000 amino acids, as well as intermediate lengths, in which the encoded XTEN can have one or more of the properties disclosed herein, when expressed as a component of a CFXTEN fusion protein. In some cases, the polynucleotide library sequences may also include additional bases used as “sequencing islands,” described more fully below.

FIG. 12 is a schematic flowchart of representative, non-limiting steps in the assembly of a XTEN polynucleotide construct and a CFXTEN polynucleotide construct in the embodiments of the invention. Individual oligonucleotides 501 are annealed into sequence motifs 502 such as a 12 amino acid motif (“12-mer”), which is ligated to additional sequence motifs from a library to create a pool that encompasses the desired length of the XTEN 504, as well as ligated to a smaller concentration of an oligo containing BbsI, and KpnI restriction sites 503. The resulting pool of ligation products is gel-purified and the band with the desired length of XTEN is cut, resulting in an isolated XTEN gene with a stopper sequence 505. The XTEN gene is cloned into a stuffer vector. In this case, the vector encodes an optional CBD sequence 506 and a GFP gene 508. Digestion is than performed with BbsI/HindIII to remove 507 and 508 and place the stop codon. The resulting product is then cloned into a BsaI/HindIII digested vector containing a gene encoding the FVIII, resulting in the gene 500 encoding an FVIII-XTEN fusion protein. A non-exhaustive list of the polynucleotides encoding XTEN and precursor sequences is provided in Tables 8-13.

TABLE 8
DNA sequences of XTEN and precursor sequences

| XTEN | |
|---|---|
| Name | DNA Nucleotide Sequence |
| AE48 | ATGGCTGAACCTGCTGGCTCTCCAACCTCCACTGAGGAAGGTACCCCGGGTAGCGGTA |
| | CTGCTTCTTCCTCTCCAGGTAGCTCTACCCCTTCTGGTGCAACCGGCTCTCCAGGTGCT |
| | TCTCCGGGCACCAGCTCTACCGGTTCT |
| AM48 | ATGGCTGAACCTGCTGGCTCTCCAACCTCCACTGAGGAAGGTGCATCCCCGGGCACCA |
| | GCTCTACCGGTTCTCCAGGTAGCTCTACCCCGTCTGGTGCTACCGGCTCTCCAGGTAGC |
| | TCTACCCCGTCTGGTGCTACTGGCTCT |
| AE144 | GGTAGCGAACCGGCAACTTCCGGCTCTGAAACCCCAGGTACTTCTGAAAGCGCTACTC |
| | CTGAGTCTGGCCCAGGTAGCGAACCTGCTACCTCTGGCTCTGAAACCCCAGGTAGCCC |
| | GGCAGGCTCTCCGACTTCCACCGAGGAAGGTACCTCTACTGAACCTTCTGAGGGTAGC |
| | GCTCCAGGTAGCGAACCGGCAACCTCTGGCTCTGAAACCCCAGGTAGCGAACCTGCTA |
| | CCTCCGGCTCTGAAACTCCAGGTAGCGAACCGGCTACTTCCGGTTCTGAAACTCCAGG |
| | TACCTCTACCGAACCTTCCGAAGGCAGCGCACCAGGTACTTCTGAAAGCGCAACCCCT |
| | GAATCCGGTCCAGGTAGCGAACCGGCTACTTCTGGCTCTGAGACTCCAGGTACTTCTA |
| | CCGAACCGTCCGAAGGTAGCGCACCA |
| AF144 | GGTACTTCTACTCCGGAAAGCGGTTCCGCATCTCCAGGTACTTCTCCTAGCGGTGAATC |
| | TTCTACTGCTCCAGGTACCTCTCCTAGCGGCGAATCTTCTACTGCTCCAGGTTCTACCA |
| | GCTCTACCGCTGAATCTCCTGGCCCAGGTTCTACCAGCGAATCCCCGTCTGGCACCGC |
| | ACCAGGTTCTACTAGCTCTACCGCAGAATCTCCGGGTCCAGGTACTTCCCCTAGCGGT |
| | GAATCTTCTACTGCTCCAGGTACCTCTACTCCGGAAAGCGGCTCCGCATCTCCAGGTTC |
| | TACTAGCTCTACTGCTGAATCTCCTGGTCCAGGTACCTCCCCTAGCGGCGAATCTTCTA |
| | CTGCTCCAGGTACCTCTCCTAGCGGCGAATCTTCTACCGCTCCAGGTACCTCCCCTAGC |
| | GGTGAATCTTCTACCGCACCA |
| AE288 | GGTACCTCTGAAAGCGCAACTCCTGAGTCTGGCCCAGGTAGCGAACCTGCTACCTCCG |
| | GCTCTGAGACTCCAGGTACCTCTGAAAGCGCAACCCCGGAATCTGGTCCAGGTAGCGA |
| | ACCTGCAACCTCTGGCTCTGAAACCCCAGGTACCTCTGAAAGCGCTACTCCTGAATCT |
| | GGCCCAGGTACTTCTACTGAACCGTCCGAGGGCAGCGCACCAGGTAGCCCTGCTGGCT |
| | CTCCAACCTCCACCGAAGAAGGTACCTCTGAAAGCGCAACCCCTGAATCCGGCCCAGG |
| | TAGCGAACCGGCAACCTCCGGTTCTGAAACCCCAGGTACTTCTGAAAGCGCTACTCCT |
| | GAGTCCGGCCCAGGTAGCCCGGCTGGCTCTCCGACTTCCACCGAGGAAGGTAGCCCGG |
| | CTGGCTCTCCAACTTCTACTGAAGAAGGTACTTCTACCGAACCTTCCGAGGGCAGCGC |
| | ACCAGGTACTTCTGAAAGCGCTACCCCTGAGTCCGGCCCAGGTACTTCTGAAAGCGCT |
| | ACTCCTGAATCCGGTCCAGGTACTTCTGAAAGCGCTACCCCGGAATCTGGCCCAGGTA |
| | GCGAACCGGCTACTTCTGGTTCTGAAACCCCAGGTAGCGAACCGGCTACCTCCGGTTC |
| | TGAAACTCCAGGTAGCCCAGCAGGCTCTCCGACTTCCACTGAGGAAGGTACTTCTACT |
| | GAACCTTCCGAAGGCAGCGCACCAGGTACCTCTACTGAACCTTCTGAGGGCAGCGCTC |
| | CAGGTAGCGAACCTGCAACCTCTGGCTCTGAAACCCCAGGTACCTCTGAAAGCGCTAC |
| | TCCTGAATCTGGCCCAGGTACTTCTACTGAACCGTCCGAGGGCAGCGCACCA |
| AE576 | GGTAGCCCGGCTGGCTCTCCTACCTCTACTGAGGAAGGTACTTCTGAAAGCGCTACTC |
| | CTGAGTCTGGTCCAGGTACCTCTACTGAACCGTCCGAAGGTAGCGCTCCAGGTAGCCC |
| | AGCAGGCTCTCCGACTTCCACTGAGGAAGGTACTTCTACTGAACCTTCCGAAGGCAGC |
| | GCACCAGGTACCTCTACTGAACCTTCTGAGGGCAGCGCTCCAGGTACTTCTGAAAGCG |
| | CTACCCCGGAATCTGGCCCAGGTAGCGAACCGGCTACTTCTGGTTCTGAAACCCCAGG |
| | TAGCGAACCGGCTACCTCCGGTTCTGAAACTCCAGGTAGCCCGGCAGGCTCTCCGACC |
| | TCTACTGAGGAAGGTACTTCTGAAAGCGCAACCCCGGAGTCCGGCCCAGGTACCTCTA |
| | CCGAACCGTCTGAGGGCAGCGCACCAGGTACTTCTACCGAACCGTCCGAGGGTAGCG |
| | CACCAGGTAGCCCAGCAGGTTCTCCTACCTCCACCGAGGAAGGTACTTCTACCGAACC |
| | GTCCGAGGGTAGCGCACCAGGTACCTCTACTGAACCTTCTGAGGGCAGCGCTCCAGGT |
| | ACTTCTGAAAGCGCTACCCCGGAGTCCGGTCCAGGTACTTCTACTGAACCGTCCGAAG |
| | GTAGCGCACCAGGTACTTCTGAAAGCGCAACCCCTGAATCCGGTCCAGGTAGCGAACC |
| | GGCTACTTCTGGCTCTGAGACTCCAGGTACTTCTACCGAACCGTCCGAAGGTAGCGCA |
| | CCAGGTACTTCTACTGAACCGTCTGAAGGTAGCGCACCAGGTACTTCTGAAAGCGCAA |
| | CCCCGGAATCCGGCCCAGGTACCTCTGAAAGCGCAACCCCGGAGTCCGGCCCAGGTA |
| | GCCCTGCTGGCTCTCCAACCTCCACCGAAGAAGGTACCTCTGAAAGCGCAACCCCTGA |
| | ATCCGGCCCAGGTAGCGAACCGGCAACCTCCGGTTCTGAAACCCCAGGTACCTCTGAA |
| | AGCGCTACTCCGGAGTCTGGCCCAGGTACCTCTACTGAACCGTCTGAGGGTAGCGCTC |
| | CAGGTACTTCTACTGAACCGTCCGAAGGTAGCGCACCAGGTACTTCTACCGAACCGTC |
| | CGAAGGCAGCGCTCCAGGTACCTCTACTGAACCTTCCGAGGGCAGCGCTCCAGGTACC |
| | TCTACCGAACCTTCTGAAGGTAGCGCACCAGGTACTTCTACCGAACCGTCCGAGGGTA |
| | GCGCACCAGGTAGCCCAGCAGGTTCTCCTACCTCCACCGAGGAAGGTACTTCTACCGA |
| | ACCGTCCGAGGGTAGCGCACCAGGTACCTCTGAAAGCGCAACTCCTGAGTCTGGCCCA |
| | GGTAGCGAACCTGCTACCTCCGGCTCTGAGACTCCAGGTACCTCTGAAAGCGCAACCC |
| | CGGAATCTGGTCCAGGTAGCGAACCTGCAACCTCTGGCTCTGAAACCCCAGGTACCTC |
| | TGAAAGCGCTACTCCTGAATCTGGCCCAGGTACTTCTACTGAACCGTCCGAGGGCAGC |
| | GCACCAGGTACTTCTGAAAGCGCTACTCCTGAGTCCGGCCCAGGTAGCCCGGCTGGCT |
| | CTCCGACTTCCACCGAGGAAGGTAGCCCGGCTGGCTCTCCAACTTCTACTGAAGAAGG |
| | TAGCCCGGCAGGCTCTCCGACCTCTACTGAGGAAGGTACTTCTGAAAGCGCAACCCCG |
| | GAGTCCGGCCCAGGTACCTCTACCGAACCGTCTGAGGGCAGCGCACCA |
| AF576 | GGTTCTACTAGCTCTACCGCTGAATCTCCTGGCCCAGGTTCCACTAGCTCTACCGCAGA |
| | ATCTCCGGGCCCAGGTTCTACTAGCGAATCCCCTTCTGGTACCGCTCCAGGTTCTACTA |
| | GCTCTACCGCTGAATCTCCGGGTCCAGGTTCTACCAGCTCTACTGCAGAATCTCCTGGC |
| | CCAGGTACTTCTACTCCGGAAAGCGGTTCCGCTTCTCCAGGTTCTACCAGCGAATCTCC |
| | TTCTGGCACCGCTCCAGGTACCTCTCCTAGCGGCGAATCTTCTACCGCTCCAGGTTCTA |
| | CTAGCGAATCTCCTTCTGGCACTGCACCAGGTTCTACCAGCGAATCTCCTTCTGGCACC |
| | GCTCCAGGTACCTCTCCTAGCGGCGAATCTTCTACCGCTCCAGGTTCTACTAGCGAATC |
| | TCCTTCTGGCACTGCACCAGGTTCTACCAGCGAATCTCCTTCTGGCACCGCTCCAGGTA |
| | CCTCTCCTAGCGGCGAATCTTCTACCGCTCCAGGTTCTACTAGCGAATCTCCTTCTGGC |
| | ACTGCACCAGGTTCTACTAGCGAATCTCCTTCTGGCACTGCACCAGGTTCTACCAGCG |
| | AATCTCCGTCTGGCACTGCACCAGGTACCTCTACCCCTGAAAGCGGTTCCGCTTCTCCA |
| | GGTTCTACTAGCGAATCTCCTTCTGGTACCGCTCCAGGTACTTCTACCCCTGAAAGCGG |
| | CTCCGCTTCTCCAGGTTCCACTAGCTCTACCGCTGAATCTCCGGGTCCAGGTTCTACTA |
| | GCTCTACTGCAGAATCTCCTGGCCCAGGTACCTCTACTCCGGAAAGCGGCTCTGCATC |
| | TCCAGGTACTTCTACCCCTGAAAGCGGTTCTGCATCTCCAGGTTCTACTAGCGAATCCC |
| | CGTCTGGTACCGCACCAGGTACTTCTACCCCGGAAAGCGGCTCTGCTTCTCCAGGTAC |
| | TTCTACCCCGGAAAGCGGCTCCGCATCTCCAGGTTCTACTAGCGAATCTCCTTCTGGTA |
| | CCGCTCCAGGTTCTACCAGCGAATCCCCGTCTGGTACTGCTCCAGGTTCTACCAGCGA |
| | ATCTCCTTCTGGTACTGCACCAGGTTCTACTAGCTCTACTGCAGAATCTCCTGGCCCAG |
| | GTACCTCTACTCCGGAAAGCGGCTCTGCATCTCCAGGTACTTCTACCCCTGAAAGCGG |
| | TTCTGCATCTCCAGGTTCTACTAGCGAATCTCCTTCTGGCACTGCACCAGGTTCTACCA |
| | GCGAATCTCCGTCTGGCACTGCACCAGGTACCTCTACCCCTGAAAGCGGTTCCGCTTCT |
| | CCAGGTTCTACTAGCGAATCTCCTTCTGGCACTGCACCAGGTTCTACCAGCGAATCTCC |
| | GTCTGGCACTGCACCAGGTACCTCTACCCCTGAAAGCGGTTCCGCTTCTCCAGGTACTT |
| | CTCCGAGCGGTGAATCTTCTACCGCACCAGGTTCTACTAGCTCTACCGCTGAATCTCCG |
| | GGCCCAGGTACTTCTCCGAGCGGTGAATCTTCTACTGCTCCAGGTTCCACTAGCTCTAC |
| | TGCTGAATCTCCTGGCCCAGGTACTTCTACTCCGGAAAGCGGTTCCGCTTCTCCAGGTT |
| | CTACTAGCGAATCTCCGTCTGGCACCGCACCAGGTTCTACTAGCTCTACTGCAGAATCT |
| | CCTGGCCCAGGTACCTCTACTCCGGAAAGCGGCTCTGCATCTCCAGGTACTTCTACCCC |
| | TGAAAGCGGTTCTGCATCTCCA |
| AE624 | ATGGCTGAACCTGCTGGCTCTCCAACCTCCACTGAGGAAGGTACCCCGGGTAGCGGTA |
| | CTGCTTCTTCCTCTCCAGGTAGCTCTACCCCTTCTGGTGCAACCGGCTCTCCAGGTGCT |
| | TCTCCGGGCACCAGCTCTACCGGTTCTCCAGGTAGCCCGGCTGGCTCTCCTACCTCTAC |
| | TGAGGAAGGTACTTCTGAAAGCGCTACTCCTGAGTCTGGTCCAGGTACCTCTACTGAA |
| | CCGTCCGAAGGTAGCGCTCCAGGTAGCCCAGCAGGCTCTCCGACTTCCACTGAGGAAG |
| | GTACTTCTACTGAACCTTCCGAAGGCAGCGCACCAGGTACCTCTACTGAACCTTCTGA |
| | GGGCAGCGCTCCAGGTACTTCTGAAAGCGCTACCCCGGAATCTGGCCCAGGTAGCGA |
| | ACCGGCTACTTCTGGTTCTGAAACCCCAGGTAGCGAACCGGCTACCTCCGGTTCTGAA |
| | ACTCCAGGTAGCCCGGCAGGCTCTCCGACCTCTACTGAGGAAGGTACTTCTGAAAGCG |
| | CAACCCCGGAGTCCGGCCCAGGTACCTCTACCGAACCGTCTGAGGGCAGCGCACCAG |
| | GTACTTCTACCGAACCGTCCGAGGGTAGCGCACCAGGTAGCCCAGCAGGTTCTCCTAC |
| | CTCCACCGAGGAAGGTACTTCTACCGAACCGTCCGAGGGTAGCGCACCAGGTACCTCT |
| | ACTGAACCTTCTGAGGGCAGCGCTCCAGGTACTTCTGAAAGCGCTACCCCGGAGTCCG |
| | GTCCAGGTACTTCTACTGAACCGTCCGAAGGTAGCGCACCAGGTACTTCTGAAAGCGC |
| | AACCCCTGAATCCGGTCCAGGTAGCGAACCGGCTACTTCTGGCTCTGAGACTCCAGGT |
| | ACTTCTACCGAACCGTCCGAAGGTAGCGCACCAGGTACTTCTACTGAACCGTCTGAAG |
| | GTAGCGCACCAGGTACTTCTGAAAGCGCAACCCCGGAATCCGGCCCAGGTACCTCTGA |
| | AAGCGCAACCCCGGAGTCCGGCCCAGGTAGCCCTGCTGGCTCTCCAACCTCCACCGAA |
| | GAAGGTACCTCTGAAAGCGCAACCCCTGAATCCGGCCCAGGTAGCGAACCGGCAACC |
| | TCCGGTTCTGAAACCCCAGGTACCTCTGAAAGCGCTACTCCGGAGTCTGGCCCAGGTA |
| | CCTCTACTGAACCGTCTGAGGGTAGCGCTCCAGGTACTTCTACTGAACCGTCCGAAGG |
| | TAGCGCACCAGGTACTTCTACCGAACCGTCCGAAGGCAGCGCTCCAGGTACCTCTACT |
| | GAACCTTCCGAGGGCAGCGCTCCAGGTACCTCTACCGAACCTTCTGAAGGTAGCGCAC |
| | CAGGTACTTCTACCGAACCGTCCGAGGGTAGCGCACCAGGTAGCCCAGCAGGTTCTCC |
| | TACCTCCACCGAGGAAGGTACTTCTACCGAACCGTCCGAGGGTAGCGCACCAGGTACC |
| | TCTGAAAGCGCAACTCCTGAGTCTGGCCCAGGTAGCGAACCTGCTACCTCCGGCTCTG |
| | AGACTCCAGGTACCTCTGAAAGCGCAACCCCGGAATCTGGTCCAGGTAGCGAACCTGC |
| | AACCTCTGGCTCTGAAACCCCAGGTACCTCTGAAAGCGCTACTCCTGAATCTGGCCCA |
| | GGTACTTCTACTGAACCGTCCGAGGGCAGCGCACCAGGTACTTCTGAAAGCGCTACTC |
| | CTGAGTCCGGCCCAGGTAGCCCGGCTGGCTCTCCGACTTCCACCGAGGAAGGTAGCCC |
| | GGCTGGCTCTCCAACTTCTACTGAAGAAGGTAGCCCGGCAGGCTCTCCGACCTCTACT |
| | GAGGAAGGTACTTCTGAAAGCGCAACCCCGGAGTCCGGCCCAGGTACCTCTACCGAA |
| | CCGTCTGAGGGCAGCGCACCA |
| AM875 | GGTACTTCTACTGAACCGTCTGAAGGCAGCGCACCAGGTAGCGAACCGGCTACTTCCG |
| | GTTCTGAAACCCCAGGTAGCCCAGCAGGTTCTCCAACTTCTACTGAAGAAGGTTCTAC |
| | CAGCTCTACCGCAGAATCTCCTGGTCCAGGTACCTCTACTCCGGAAAGCGGCTCTGCA |
| | TCTCCAGGTTCTACTAGCGAATCTCCTTCTGGCACTGCACCAGGTTCTACTAGCGAATC |
| | CCCGTCTGGTACTGCTCCAGGTACTTCTACTCCTGAAAGCGGTTCCGCTTCTCCAGGTA |
| | CCTCTACTCCGGAAAGCGGTTCTGCATCTCCAGGTAGCGAACCGGCAACCTCCGGCTC |
| | TGAAACCCCAGGTACCTCTGAAAGCGCTACTCCTGAATCCGGCCCAGGTAGCCCGGCA |
| | GGTTCTCCGACTTCCACTGAGGAAGGTACCTCTACTGAACCTTCTGAGGGCAGCGCTC |
| | CAGGTACTTCTGAAAGCGCTACCCCGGAGTCCGGTCCAGGTACTTCTACTGAACCGTC |
| | CGAAGGTAGCGCACCAGGTACTTCTACCGAACCGTCCGAGGGTAGCGCACCAGGTAG |
| | CCCAGCAGGTTCTCCTACCTCCACCGAGGAAGGTACTTCTACCGAACCGTCCGAGGGT |
| | AGCGCACCAGGTACTTCTACCGAACCTTCCGAGGGCAGCGCACCAGGTACTTCTGAAA |
| | GCGCTACCCCTGAGTCCGGCCCAGGTACTTCTGAAAGCGCTACTCCTGAATCCGGTCC |
| | AGGTACCTCTACTGAACCTTCCGAAGGCAGCGCTCCAGGTACCTCTACCGAACCGTCC |
| | GAGGGCAGCGCACCAGGTACTTCTGAAAGCGCAACCCCTGAATCCGGTCCAGGTACTT |
| | CTACTGAACCTTCCGAAGGTAGCGCTCCAGGTAGCGAACCTGCTACTTCTGGTTCTGA |
| | AACCCCAGGTAGCCCGGCTGGCTCTCCGACCTCCACCGAGGAAGGTAGCTCTACCCCG |
| | TCTGGTGCTACTGGTTCTCCAGGTACTCCGGGCAGCGGTACTGCTTCTTCCTCTCCAGG |
| | TAGCTCTACCCCTTCTGGTGCTACTGGCTCTCCAGGTACCTCTACCGAACCGTCCGAGG |
| | GTAGCGCACCAGGTACCTCTACTGAACCGTCTGAGGGTAGCGCTCCAGGTAGCGAACC |
| | GGCAACCTCCGGTTCTGAAACTCCAGGTAGCCCTGCTGGCTCTCCGACTTCTACTGAG |
| | GAAGGTAGCCCGGCTGGTTCTCCGACTTCTACTGAGGAAGGTACTTCTACCGAACCTT |
| | CCGAAGGTAGCGCTCCAGGTGCAAGCGCAAGCGGCGCGCCAAGCACGGGAGGTACTT |
| | CTGAAAGCGCTACTCCTGAGTCCGGCCCAGGTAGCCCGGCTGGCTCTCCGACTTCCAC |
| | CGAGGAAGGTAGCCCGGCTGGCTCTCCAACTTCTACTGAAGAAGGTTCTACCAGCTCT |
| | ACCGCTGAATCTCCTGGCCCAGGTTCTACTAGCGAATCTCCGTCTGGCACCGCACCAG |
| | GTACTTCCCCTAGCGGTGAATCTTCTACTGCACCAGGTACCCCTGGCAGCGGTACCGC |
| | TTCTTCCTCTCCAGGTAGCTCTACCCCGTCTGGTGCTACTGGCTCTCCAGGTTCTAGCC |
| | CGTCTGCATCTACCGGTACCGGCCCAGGTAGCGAACCGGCAACCTCCGGCTCTGAAAC |
| | TCCAGGTACTTCTGAAAGCGCTACTCCGGAATCCGGCCCAGGTAGCGAACCGGCTACT |
| | TCCGGCTCTGAAACCCCAGGTTCCACCAGCTCTACTGCAGAATCTCCGGGCCCAGGTT |
| | CTACTAGCTCTACTGCAGAATCTCCGGGTCCAGGTACTTCTCCTAGCGGCGAATCTTCT |
| | ACCGCTCCAGGTAGCGAACCGGCAACCTCTGGCTCTGAAACTCCAGGTAGCGAACCTG |
| | CAACCTCCGGCTCTGAAACCCCAGGTACTTCTACTGAACCTTCTGAGGGCAGCGCACC |
| | AGGTTCTACCAGCTCTACCGCAGAATCTCCTGGTCCAGGTACCTCTACTCCGGAAAGC |
| | GGCTCTGCATCTCCAGGTTCTACTAGCGAATCTCCTTCTGGCACTGCACCAGGTACTTC |
| | TACCGAACCGTCCGAAGGCAGCGCTCCAGGTACCTCTACTGAACCTTCCGAGGGCAGC |
| | GCTCCAGGTACCTCTACCGAACCTTCTGAAGGTAGCGCACCAGGTAGCTCTACTCCGT |
| | CTGGTGCAACCGGCTCCCCAGGTTCTAGCCCGTCTGCTTCCACTGGTACTGGCCCAGGT |
| | GCTTCCCCGGGCACCAGCTCTACTGGTTCTCCAGGTAGCGAACCTGCTACCTCCGGTTC |
| | TGAAACCCCAGGTACCTCTGAAAGCGCAACTCCGGAGTCTGGTCCAGGTAGCCCTGCA |
| | GGTTCTCCTACCTCCACTGAGGAAGGTAGCTCTACTCCGTCTGGTGCAACCGGCTCCCC |
| | AGGTTCTAGCCCGTCTGCTTCCACTGGTACTGGCCCAGGTGCTTCCCCGGGCACCAGCT |
| | CTACTGGTTCTCCAGGTACCTCTGAAAGCGCTACTCCGGAGTCTGGCCCAGGTACCTCT |
| | ACTGAACCGTCTGAGGGTAGCGCTCCAGGTACTTCTACTGAACCGTCCGAAGGTAGCG |
| | CACCA |
| AE864 | GGTAGCCCGGCTGGCTCTCCTACCTCTACTGAGGAAGGTACTTCTGAAAGCGCTACTC |
| | CTGAGTCTGGTCCAGGTACCTCTACTGAACCGTCCGAAGGTAGCGCTCCAGGTAGCCC |
| | AGCAGGCTCTCCGACTTCCACTGAGGAAGGTACTTCTACTGAACCTTCCGAAGGCAGC |
| | GCACCAGGTACCTCTACTGAACCTTCTGAGGGCAGCGCTCCAGGTACTTCTGAAAGCG |
| | CTACCCCGGAATCTGGCCCAGGTAGCGAACCGGCTACTTCTGGTTCTGAAACCCCAGG |
| | TAGCGAACCGGCTACCTCCGGTTCTGAAACTCCAGGTAGCCCGGCAGGCTCTCCGACC |
| | TCTACTGAGGAAGGTACTTCTGAAAGCGCAACCCCGGAGTCCGGCCCAGGTACCTCTA |
| | CCGAACCGTCTGAGGGCAGCGCACCAGGTACTTCTACCGAACCGTCCGAGGGTAGCG |
| | CACCAGGTAGCCCAGCAGGTTCTCCTACCTCCACCGAGGAAGGTACTTCTACCGAACC |
| | GTCCGAGGGTAGCGCACCAGGTACCTCTACTGAACCTTCTGAGGGCAGCGCTCCAGGT |
| | ACTTCTGAAAGCGCTACCCCGGAGTCCGGTCCAGGTACTTCTACTGAACCGTCCGAAG |
| | GTAGCGCACCAGGTACTTCTGAAAGCGCAACCCCTGAATCCGGTCCAGGTAGCGAACC |
| | GGCTACTTCTGGCTCTGAGACTCCAGGTACTTCTACCGAACCGTCCGAAGGTAGCGCA |
| | CCAGGTACTTCTACTGAACCGTCTGAAGGTAGCGCACCAGGTACTTCTGAAAGCGCAA |
| | CCCCGGAATCCGGCCCAGGTACCTCTGAAAGCGCAACCCCGGAGTCCGGCCCAGGTA |
| | GCCCTGCTGGCTCTCCAACCTCCACCGAAGAAGGTACCTCTGAAAGCGCAACCCCTGA |
| | ATCCGGCCCAGGTAGCGAACCGGCAACCTCCGGTTCTGAAACCCCAGGTACCTCTGAA |
| | AGCGCTACTCCGGAGTCTGGCCCAGGTACCTCTACTGAACCGTCTGAGGGTAGCGCTC |
| | CAGGTACTTCTACTGAACCGTCCGAAGGTAGCGCACCAGGTACTTCTACCGAACCGTC |
| | CGAAGGCAGCGCTCCAGGTACCTCTACTGAACCTTCCGAGGGCAGCGCTCCAGGTACC |
| | TCTACCGAACCTTCTGAAGGTAGCGCACCAGGTACTTCTACCGAACCGTCCGAGGGTA |
| | GCGCACCAGGTAGCCCAGCAGGTTCTCCTACCTCCACCGAGGAAGGTACTTCTACCGA |
| | ACCGTCCGAGGGTAGCGCACCAGGTACCTCTGAAAGCGCAACTCCTGAGTCTGGCCCA |
| | GGTAGCGAACCTGCTACCTCCGGCTCTGAGACTCCAGGTACCTCTGAAAGCGCAACCC |
| | CGGAATCTGGTCCAGGTAGCGAACCTGCAACCTCTGGCTCTGAAACCCCAGGTACCTC |
| | TGAAAGCGCTACTCCTGAATCTGGCCCAGGTACTTCTACTGAACCGTCCGAGGGCAGC |
| | GCACCAGGTACTTCTGAAAGCGCTACTCCTGAGTCCGGCCCAGGTAGCCCGGCTGGCT |
| | CTCCGACTTCCACCGAGGAAGGTAGCCCGGCTGGCTCTCCAACTTCTACTGAAGAAGG |
| | TAGCCCGGCAGGCTCTCCGACCTCTACTGAGGAAGGTACTTCTGAAAGCGCAACCCCG |
| | GAGTCCGGCCCAGGTACCTCTACCGAACCGTCTGAGGGCAGCGCACCAGGTACCTCTG |
| | AAAGCGCAACTCCTGAGTCTGGCCCAGGTAGCGAACCTGCTACCTCCGGCTCTGAGAC |
| | TCCAGGTACCTCTGAAAGCGCAACCCCGGAATCTGGTCCAGGTAGCGAACCTGCAACC |
| | TCTGGCTCTGAAACCCCAGGTACCTCTGAAAGCGCTACTCCTGAATCTGGCCCAGGTA |
| | CTTCTACTGAACCGTCCGAGGGCAGCGCACCAGGTAGCCCTGCTGGCTCTCCAACCTC |
| | CACCGAAGAAGGTACCTCTGAAAGCGCAACCCCTGAATCCGGCCCAGGTAGCGAACC |
| | GGCAACCTCCGGTTCTGAAACCCCAGGTACTTCTGAAAGCGCTACTCCTGAGTCCGGC |
| | CCAGGTAGCCCGGCTGGCTCTCCGACTTCCACCGAGGAAGGTAGCCCGGCTGGCTCTC |
| | CAACTTCTACTGAAGAAGGTACTTCTACCGAACCTTCCGAGGGCAGCGCACCAGGTAC |
| | TTCTGAAAGCGCTACCCCTGAGTCCGGCCCAGGTACTTCTGAAAGCGCTACTCCTGAA |
| | TCCGGTCCAGGTACTTCTGAAAGCGCTACCCCGGAATCTGGCCCAGGTAGCGAACCGG |
| | CTACTTCTGGTTCTGAAACCCCAGGTAGCGAACCGGCTACCTCCGGTTCTGAAACTCC |
| | AGGTAGCCCAGCAGGCTCTCCGACTTCCACTGAGGAAGGTACTTCTACTGAACCTTCC |
| | GAAGGCAGCGCACCAGGTACCTCTACTGAACCTTCTGAGGGCAGCGCTCCAGGTAGC |
| | GAACCTGCAACCTCTGGCTCTGAAACCCCAGGTACCTCTGAAAGCGCTACTCCTGAAT |
| | CTGGCCCAGGTACTTCTACTGAACCGTCCGAGGGCAGCGCACCA |
| AF864 | GGTTCTACCAGCGAATCTCCTTCTGGCACCGCTCCAGGTACCTCTCCTAGCGGCGAATC |
| | TTCTACCGCTCCAGGTTCTACTAGCGAATCTCCTTCTGGCACTGCACCAGGTTCTACTA |
| | GCGAATCCCCGTCTGGTACTGCTCCAGGTACTTCTACTCCTGAAAGCGGTTCCGCTTCT |
| | CCAGGTACCTCTACTCCGGAAAGCGGTTCTGCATCTCCAGGTTCTACCAGCGAATCTC |
| | CTTCTGGCACCGCTCCAGGTTCTACTAGCGAATCCCCGTCTGGTACCGCACCAGGTACT |
| | TCTCCTAGCGGCGAATCTTCTACCGCACCAGGTTCTACTAGCGAATCTCCGTCTGGCAC |
| | TGCTCCAGGTACTTCTCCTAGCGGTGAATCTTCTACCGCTCCAGGTACTTCCCCTAGCG |
| | GCGAATCTTCTACCGCTCCAGGTTCTACTAGCTCTACTGCAGAATCTCCGGGCCCAGGT |
| | ACCTCTCCTAGCGGTGAATCTTCTACCGCTCCAGGTACTTCTCCGAGCGGTGAATCTTC |
| | TACCGCTCCAGGTTCTACTAGCTCTACTGCAGAATCTCCTGGCCCAGGTACCTCTACTC |
| | CGGAAAGCGGCTCTGCATCTCCAGGTACTTCTACCCCTGAAAGCGGTTCTGCATCTCC |
| | AGGTTCTACTAGCGAATCTCCTTCTGGCACTGCACCAGGTTCTACCAGCGAATCTCCGT |
| | CTGGCACTGCACCAGGTACCTCTACCCCTGAAAGCGGTTCCGCTTCTCCAGGTTCTACC |
| | AGCTCTACCGCAGAATCTCCTGGTCCAGGTACCTCTACTCCGGAAAGCGGCTCTGCAT |
| | CTCCAGGTTCTACTAGCGAATCTCCTTCTGGCACTGCACCAGGTACTTCTCCGAGCGGT |
| | GAATCTTCTACCGCACCAGGTTCTACTAGCTCTACCGCTGAATCTCCGGGCCCAGGTA |
| | CTTCTCCGAGCGGTGAATCTTCTACTGCTCCAGGTACCTCTACTCCTGAAAGCGGTTCT |
| | GCATCTCCAGGTTCCACTAGCTCTACCGCAGAATCTCCGGGCCCAGGTTCTACTAGCTC |
| | TACTGCTGAATCTCCTGGCCCAGGTTCTACTAGCTCTACTGCTGAATCTCCGGGTCCAG |
| | GTTCTACCAGCTCTACTGCTGAATCTCCTGGTCCAGGTACCTCCCCGAGCGGTGAATCT |
| | TCTACTGCACCAGGTTCTACTAGCGAATCTCCTTCTGGCACTGCACCAGGTTCTACCAG |
| | CGAATCTCCGTCTGGCACTGCACCAGGTACCTCTACCCCTGAAAGCGGTCCXXXXXXX |
| | XXXXXTGCAAGCGCAAGCGGCGCGCCAAGCACGGGAXXXXXXXXTAGCGAATCTCCT |
| | TCTGGTACCGCTCCAGGTTCTACCAGCGAATCCCCGTCTGGTACTGCTCCAGGTTCTAC |
| | CAGCGAATCTCCTTCTGGTACTGCACCAGGTTCTACTAGCGAATCTCCTTCTGGTACCG |
| | CTCCAGGTTCTACCAGCGAATCCCCGTCTGGTACTGCTCCAGGTTCTACCAGCGAATCT |
| | CCTTCTGGTACTGCACCAGGTACTTCTACTCCGGAAAGCGGTTCCGCATCTCCAGGTAC |
| | TTCTCCTAGCGGTGAATCTTCTACTGCTCCAGGTACCTCTCCTAGCGGCGAATCTTCTA |
| | CTGCTCCAGGTTCTACCAGCTCTACTGCTGAATCTCCGGGTCCAGGTACTTCCCCGAGC |
| | GGTGAATCTTCTACTGCACCAGGTACTTCTACTCCGGAAAGCGGTTCCGCTTCTCCAGG |
| | TTCTACCAGCGAATCTCCTTCTGGCACCGCTCCAGGTTCTACTAGCGAATCCCCGTCTG |
| | GTACCGCACCAGGTACTTCTCCTAGCGGCGAATCTTCTACCGCACCAGGTTCTACTAG |
| | CGAATCCCCGTCTGGTACCGCACCAGGTACTTCTACCCCGGAAAGCGGCTCTGCTTCT |
| | CCAGGTACTTCTACCCCGGAAAGCGGCTCCGCATCTCCAGGTTCTACTAGCGAATCTC |
| | CTTCTGGTACCGCTCCAGGTACTTCTACCCCTGAAAGCGGCTCCGCTTCTCCAGGTTCC |
| | ACTAGCTCTACCGCTGAATCTCCGGGTCCAGGTTCTACCAGCGAATCTCCTTCTGGCAC |
| | CGCTCCAGGTTCTACTAGCGAATCCCCGTCTGGTACCGCACCAGGTACTTCTCCTAGCG |
| | GCGAATCTTCTACCGCACCAGGTTCTACCAGCTCTACTGCTGAATCTCCGGGTCCAGGT |
| | ACTTCCCCGAGCGGTGAATCTTCTACTGCACCAGGTACTTCTACTCCGGAAAGCGGTT |
| | CCGCTTCTCCAGGTACCTCCCCTAGCGGCGAATCTTCTACTGCTCCAGGTACCTCTCCT |
| | AGCGGCGAATCTTCTACCGCTCCAGGTACCTCCCCTAGCGGTGAATCTTCTACCGCAC |
| | CAGGTTCTACTAGCTCTACTGCTGAATCTCCGGGTCCAGGTTCTACCAGCTCTACTGCT |
| | GAATCTCCTGGTCCAGGTACCTCCCCGAGCGGTGAATCTTCTACTGCACCAGGTTCTA |
| | GCCCTTCTGCTTCCACCGGTACCGGCCCAGGTAGCTCTACTCCGTCTGGTGCAACTGGC |
| | TCTCCAGGTAGCTCTACTCCGTCTGGTGCAACCGGCTCCCCA |
| | XXXX was inserted in two areas where no sequence information |
| | is available. |
| AG864 | GGTGCTTCCCCGGGCACCAGCTCTACTGGTTCTCCAGGTTCTAGCCCGTCTGCTTCTAC |
| | TGGTACTGGTCCAGGTTCTAGCCCTTCTGCTTCCACTGGTACTGGTCCAGGTACCCCGG |
| | GTAGCGGTACCGCTTCTTCTTCTCCAGGTAGCTCTACTCCGTCTGGTGCTACCGGCTCT |
| | CCAGGTTCTAACCCTTCTGCATCCACCGGTACCGGCCCAGGTGCTTCTCCGGGCACCA |
| | GCTCTACTGGTTCTCCAGGTACCCCGGGCAGCGGTACCGCATCTTCTTCTCCAGGTAGC |
| | TCTACTCCTTCTGGTGCAACTGGTTCTCCAGGTACTCCTGGCAGCGGTACCGCTTCTTC |
| | TTCTCCAGGTGCTTCTCCTGGTACTAGCTCTACTGGTTCTCCAGGTGCTTCTCCGGGCA |
| | CTAGCTCTACTGGTTCTCCAGGTACCCCGGGTAGCGGTACTGCTTCTTCCTCTCCAGGT |
| | AGCTCTACCCCTTCTGGTGCAACCGGCTCTCCAGGTGCTTCTCCGGGCACCAGCTCTAC |
| | CGGTTCTCCAGGTACCCCGGGTAGCGGTACCGCTTCTTCTTCTCCAGGTAGCTCTACTC |
| | CGTCTGGTGCTACCGGCTCTCCAGGTTCTAACCCTTCTGCATCCACCGGTACCGGCCCA |
| | GGTTCTAGCCCTTCTGCTTCCACCGGTACTGGCCCAGGTAGCTCTACCCCTTCTGGTGC |
| | TACCGGCTCCCCAGGTAGCTCTACTCCTTCTGGTGCAACTGGCTCTCCAGGTGCATCTC |
| | CGGGCACTAGCTCTACTGGTTCTCCAGGTGCATCCCCTGGCACTAGCTCTACTGGTTCT |
| | CCAGGTGCTTCTCCTGGTACCAGCTCTACTGGTTCTCCAGGTACTCCTGGCAGCGGTAC |
| | CGCTTCTTCTTCTCCAGGTGCTTCTCCTGGTACTAGCTCTACTGGTTCTCCAGGTGCTTC |
| | TCCGGGCACTAGCTCTACTGGTTCTCCAGGTGCTTCCCCGGGCACTAGCTCTACCGGTT |
| | CTCCAGGTTCTAGCCCTTCTGCATCTACTGGTACTGGCCCAGGTACTCCGGGCAGCGGT |
| | ACTGCTTCTTCCTCTCCAGGTGCATCTCCGGGCACTAGCTCTACTGGTTCTCCAGGTGC |
| | ATCCCCTGGCACTAGCTCTACTGGTTCTCCAGGTGCTTCTCCTGGTACCAGCTCTACTG |
| | GTTCTCCAGGTAGCTCTACTCCGTCTGGTGCAACCGGTTCCCCAGGTAGCTCTACTCCT |
| | TCTGGTGCTACTGGCTCCCCAGGTGCATCCCCTGGCACCAGCTCTACCGGTTCTCCAGG |
| | TACCCCGGGCAGCGGTACCGCATCTTCCTCTCCAGGTAGCTCTACCCCGTCTGGTGCTA |
| | CCGGTTCCCCAGGTAGCTCTACCCCGTCTGGTGCAACCGGCTCCCCAGGTAGCTCTACT |
| | CCGTCTGGTGCAACCGGCTCCCCAGGTTCTAGCCCGTCTGCTTCCACTGGTACTGGCCC |
| | AGGTGCTTCCCCGGGCACCAGCTCTACTGGTTCTCCAGGTGCATCCCCGGGTACCAGC |
| | TCTACCGGTTCTCCAGGTACTCCTGGCAGCGGTACTGCATCTTCCTCTCCAGGTGCTTC |
| | TCCGGGCACCAGCTCTACTGGTTCTCCAGGTGCATCTCCGGGCACTAGCTCTACTGGTT |
| | CTCCAGGTGCATCCCCTGGCACTAGCTCTACTGGTTCTCCAGGTGCTTCTCCTGGTACC |
| | AGCTCTACTGGTTCTCCAGGTACCCCTGGTAGCGGTACTGCTTCTTCCTCTCCAGGTAG |
| | CTCTACTCCGTCTGGTGCTACCGGTTCTCCAGGTACCCCGGGTAGCGGTACCGCATCTT |
| | CTTCTCCAGGTAGCTCTACCCCGTCTGGTGCTACTGGTTCTCCAGGTACTCCGGGCAGC |
| | GGTACTGCTTCTTCCTCTCCAGGTAGCTCTACCCCTTCTGGTGCTACTGGCTCTCCAGG |
| | TAGCTCTACCCCGTCTGGTGCTACTGGCTCCCCAGGTTCTAGCCCTTCTGCATCCACCG |
| | GTACCGGTCCAGGTTCTAGCCCGTCTGCATCTACTGGTACTGGTCCAGGTGCATCCCCG |
| | GGCACTAGCTCTACCGGTTCTCCAGGTACTCCTGGTAGCGGTACTGCTTCTTCTTCTCC |
| | AGGTAGCTCTACTCCTTCTGGTGCTACTGGTTCTCCAGGTTCTAGCCCTTCTGCATCCA |
| | CCGGTACCGGCCCAGGTTCTAGCCCGTCTGCTTCTACCGGTACTGGTCCAGGTGCTTCT |
| | CCGGGTACTAGCTCTACTGGTTCTCCAGGTGCATCTCCTGGTACTAGCTCTACTGGTTC |
| | TCCAGGTAGCTCTACTCCGTCTGGTGCAACCGGCTCTCCAGGTTCTAGCCCTTCTGCAT |
| | CTACCGGTACTGGTCCAGGTGCATCCCCTGGTACCAGCTCTACCGGTTCTCCAGGTTCT |
| | AGCCCTTCTGCTTCTACCGGTACCGGTCCAGGTACCCCTGGCAGCGGTACCGCATCTTC |
| | CTCTCCAGGTAGCTCTACTCCGTCTGGTGCAACCGGTTCCCCAGGTAGCTCTACTCCTT |
| | CTGGTGCTACTGGCTCCCCAGGTGCATCCCCTGGCACCAGCTCTACCGGTTCTCCA |
| AM923 | ATGGCTGAACCTGCTGGCTCTCCAACCTCCACTGAGGAAGGTGCATCCCCGGGCACCA |
| | GCTCTACCGGTTCTCCAGGTAGCTCTACCCCGTCTGGTGCTACCGGCTCTCCAGGTAGC |
| | TCTACCCCGTCTGGTGCTACTGGCTCTCCAGGTACTTCTACTGAACCGTCTGAAGGCAG |
| | CGCACCAGGTAGCGAACCGGCTACTTCCGGTTCTGAAACCCCAGGTAGCCCAGCAGGT |
| | TCTCCAACTTCTACTGAAGAAGGTTCTACCAGCTCTACCGCAGAATCTCCTGGTCCAG |
| | GTACCTCTACTCCGGAAAGCGGCTCTGCATCTCCAGGTTCTACTAGCGAATCTCCTTCT |
| | GGCACTGCACCAGGTTCTACTAGCGAATCCCCGTCTGGTACTGCTCCAGGTACTTCTAC |
| | TCCTGAAAGCGGTTCCGCTTCTCCAGGTACCTCTACTCCGGAAAGCGGTTCTGCATCTC |
| | CAGGTAGCGAACCGGCAACCTCCGGCTCTGAAACCCCAGGTACCTCTGAAAGCGCTAC |
| | TCCTGAATCCGGCCCAGGTAGCCCGGCAGGTTCTCCGACTTCCACTGAGGAAGGTACC |
| | TCTACTGAACCTTCTGAGGGCAGCGCTCCAGGTACTTCTGAAAGCGCTACCCCGGAGT |
| | CCGGTCCAGGTACTTCTACTGAACCGTCCGAAGGTAGCGCACCAGGTACTTCTACCGA |
| | ACCGTCCGAGGGTAGCGCACCAGGTAGCCCAGCAGGTTCTCCTACCTCCACCGAGGAA |
| | GGTACTTCTACCGAACCGTCCGAGGGTAGCGCACCAGGTACTTCTACCGAACCTTCCG |
| | AGGGCAGCGCACCAGGTACTTCTGAAAGCGCTACCCCTGAGTCCGGCCCAGGTACTTC |
| | TGAAAGCGCTACTCCTGAATCCGGTCCAGGTACCTCTACTGAACCTTCCGAAGGCAGC |
| | GCTCCAGGTACCTCTACCGAACCGTCCGAGGGCAGCGCACCAGGTACTTCTGAAAGCG |
| | CAACCCCTGAATCCGGTCCAGGTACTTCTACTGAACCTTCCGAAGGTAGCGCTCCAGG |
| | TAGCGAACCTGCTACTTCTGGTTCTGAAACCCCAGGTAGCCCGGCTGGCTCTCCGACC |
| | TCCACCGAGGAAGGTAGCTCTACCCCGTCTGGTGCTACTGGTTCTCCAGGTACTCCGG |
| | GCAGCGGTACTGCTTCTTCCTCTCCAGGTAGCTCTACCCCTTCTGGTGCTACTGGCTCT |
| | CCAGGTACCTCTACCGAACCGTCCGAGGGTAGCGCACCAGGTACCTCTACTGAACCGT |
| | CTGAGGGTAGCGCTCCAGGTAGCGAACCGGCAACCTCCGGTTCTGAAACTCCAGGTAG |
| | CCCTGCTGGCTCTCCGACTTCTACTGAGGAAGGTAGCCCGGCTGGTTCTCCGACTTCTA |
| | CTGAGGAAGGTACTTCTACCGAACCTTCCGAAGGTAGCGCTCCAGGTGCAAGCGCAA |
| | GCGGCGCGCCAAGCACGGGAGGTACTTCTGAAAGCGCTACTCCTGAGTCCGGCCCAG |
| | GTAGCCCGGCTGGCTCTCCGACTTCCACCGAGGAAGGTAGCCCGGCTGGCTCTCCAAC |
| | TTCTACTGAAGAAGGTTCTACCAGCTCTACCGCTGAATCTCCTGGCCCAGGTTCTACTA |
| | GCGAATCTCCGTCTGGCACCGCACCAGGTACTTCCCCTAGCGGTGAATCTTCTACTGC |
| | ACCAGGTACCCCTGGCAGCGGTACCGCTTCTTCCTCTCCAGGTAGCTCTACCCCGTCTG |
| | GTGCTACTGGCTCTCCAGGTTCTAGCCCGTCTGCATCTACCGGTACCGGCCCAGGTAG |
| | CGAACCGGCAACCTCCGGCTCTGAAACTCCAGGTACTTCTGAAAGCGCTACTCCGGAA |
| | TCCGGCCCAGGTAGCGAACCGGCTACTTCCGGCTCTGAAACCCCAGGTTCCACCAGCT |
| | CTACTGCAGAATCTCCGGGCCCAGGTTCTACTAGCTCTACTGCAGAATCTCCGGGTCC |
| | AGGTACTTCTCCTAGCGGCGAATCTTCTACCGCTCCAGGTAGCGAACCGGCAACCTCT |
| | GGCTCTGAAACTCCAGGTAGCGAACCTGCAACCTCCGGCTCTGAAACCCCAGGTACTT |
| | CTACTGAACCTTCTGAGGGCAGCGCACCAGGTTCTACCAGCTCTACCGCAGAATCTCC |
| | TGGTCCAGGTACCTCTACTCCGGAAAGCGGCTCTGCATCTCCAGGTTCTACTAGCGAA |
| | TCTCCTTCTGGCACTGCACCAGGTACTTCTACCGAACCGTCCGAAGGCAGCGCTCCAG |
| | GTACCTCTACTGAACCTTCCGAGGGCAGCGCTCCAGGTACCTCTACCGAACCTTCTGA |
| | AGGTAGCGCACCAGGTAGCTCTACTCCGTCTGGTGCAACCGGCTCCCCAGGTTCTAGC |
| | CCGTCTGCTTCCACTGGTACTGGCCCAGGTGCTTCCCCGGGCACCAGCTCTACTGGTTC |
| | TCCAGGTAGCGAACCTGCTACCTCCGGTTCTGAAACCCCAGGTACCTCTGAAAGCGCA |
| | ACTCCGGAGTCTGGTCCAGGTAGCCCTGCAGGTTCTCCTACCTCCACTGAGGAAGGTA |
| | GCTCTACTCCGTCTGGTGCAACCGGCTCCCCAGGTTCTAGCCCGTCTGCTTCCACTGGT |
| | ACTGGCCCAGGTGCTTCCCCGGGCACCAGCTCTACTGGTTCTCCAGGTACCTCTGAAA |
| | GCGCTACTCCGGAGTCTGGCCCAGGTACCTCTACTGAACCGTCTGAGGGTAGCGCTCC |
| | AGGTACTTCTACTGAACCGTCCGAAGGTAGCGCACCA |
| AE912 | ATGGCTGAACCTGCTGGCTCTCCAACCTCCACTGAGGAAGGTACCCCGGGTAGCGGTA |
| | CTGCTTCTTCCTCTCCAGGTAGCTCTACCCCTTCTGGTGCAACCGGCTCTCCAGGTGCT |
| | TCTCCGGGCACCAGCTCTACCGGTTCTCCAGGTAGCCCGGCTGGCTCTCCTACCTCTAC |
| | TGAGGAAGGTACTTCTGAAAGCGCTACTCCTGAGTCTGGTCCAGGTACCTCTACTGAA |
| | CCGTCCGAAGGTAGCGCTCCAGGTAGCCCAGCAGGCTCTCCGACTTCCACTGAGGAAG |
| | GTACTTCTACTGAACCTTCCGAAGGCAGCGCACCAGGTACCTCTACTGAACCTTCTGA |
| | GGGCAGCGCTCCAGGTACTTCTGAAAGCGCTACCCCGGAATCTGGCCCAGGTAGCGA |
| | ACCGGCTACTTCTGGTTCTGAAACCCCAGGTAGCGAACCGGCTACCTCCGGTTCTGAA |
| | ACTCCAGGTAGCCCGGCAGGCTCTCCGACCTCTACTGAGGAAGGTACTTCTGAAAGCG |
| | CAACCCCGGAGTCCGGCCCAGGTACCTCTACCGAACCGTCTGAGGGCAGCGCACCAG |
| | GTACTTCTACCGAACCGTCCGAGGGTAGCGCACCAGGTAGCCCAGCAGGTTCTCCTAC |
| | CTCCACCGAGGAAGGTACTTCTACCGAACCGTCCGAGGGTAGCGCACCAGGTACCTCT |
| | ACTGAACCTTCTGAGGGCAGCGCTCCAGGTACTTCTGAAAGCGCTACCCCGGAGTCCG |
| | GTCCAGGTACTTCTACTGAACCGTCCGAAGGTAGCGCACCAGGTACTTCTGAAAGCGC |
| | AACCCCTGAATCCGGTCCAGGTAGCGAACCGGCTACTTCTGGCTCTGAGACTCCAGGT |
| | ACTTCTACCGAACCGTCCGAAGGTAGCGCACCAGGTACTTCTACTGAACCGTCTGAAG |
| | GTAGCGCACCAGGTACTTCTGAAAGCGCAACCCCGGAATCCGGCCCAGGTACCTCTGA |
| | AAGCGCAACCCCGGAGTCCGGCCCAGGTAGCCCTGCTGGCTCTCCAACCTCCACCGAA |
| | GAAGGTACCTCTGAAAGCGCAACCCCTGAATCCGGCCCAGGTAGCGAACCGGCAACC |
| | TCCGGTTCTGAAACCCCAGGTACCTCTGAAAGCGCTACTCCGGAGTCTGGCCCAGGTA |
| | CCTCTACTGAACCGTCTGAGGGTAGCGCTCCAGGTACTTCTACTGAACCGTCCGAAGG |
| | TAGCGCACCAGGTACTTCTACCGAACCGTCCGAAGGCAGCGCTCCAGGTACCTCTACT |
| | GAACCTTCCGAGGGCAGCGCTCCAGGTACCTCTACCGAACCTTCTGAAGGTAGCGCAC |
| | CAGGTACTTCTACCGAACCGTCCGAGGGTAGCGCACCAGGTAGCCCAGCAGGTTCTCC |
| | TACCTCCACCGAGGAAGGTACTTCTACCGAACCGTCCGAGGGTAGCGCACCAGGTACC |
| | TCTGAAAGCGCAACTCCTGAGTCTGGCCCAGGTAGCGAACCTGCTACCTCCGGCTCTG |
| | AGACTCCAGGTACCTCTGAAAGCGCAACCCCGGAATCTGGTCCAGGTAGCGAACCTGC |
| | AACCTCTGGCTCTGAAACCCCAGGTACCTCTGAAAGCGCTACTCCTGAATCTGGCCCA |
| | GGTACTTCTACTGAACCGTCCGAGGGCAGCGCACCAGGTACTTCTGAAAGCGCTACTC |
| | CTGAGTCCGGCCCAGGTAGCCCGGCTGGCTCTCCGACTTCCACCGAGGAAGGTAGCCC |
| | GGCTGGCTCTCCAACTTCTACTGAAGAAGGTAGCCCGGCAGGCTCTCCGACCTCTACT |
| | GAGGAAGGTACTTCTGAAAGCGCAACCCCGGAGTCCGGCCCAGGTACCTCTACCGAA |
| | CCGTCTGAGGGCAGCGCACCAGGTACCTCTGAAAGCGCAACTCCTGAGTCTGGCCCAG |
| | GTAGCGAACCTGCTACCTCCGGCTCTGAGACTCCAGGTACCTCTGAAAGCGCAACCCC |
| | GGAATCTGGTCCAGGTAGCGAACCTGCAACCTCTGGCTCTGAAACCCCAGGTACCTCT |
| | GAAAGCGCTACTCCTGAATCTGGCCCAGGTACTTCTACTGAACCGTCCGAGGGCAGCG |
| | CACCAGGTAGCCCTGCTGGCTCTCCAACCTCCACCGAAGAAGGTACCTCTGAAAGCGC |
| | AACCCCTGAATCCGGCCCAGGTAGCGAACCGGCAACCTCCGGTTCTGAAACCCCAGGT |
| | ACTTCTGAAAGCGCTACTCCTGAGTCCGGCCCAGGTAGCCCGGCTGGCTCTCCGACTT |
| | CCACCGAGGAAGGTAGCCCGGCTGGCTCTCCAACTTCTACTGAAGAAGGTACTTCTAC |
| | CGAACCTTCCGAGGGCAGCGCACCAGGTACTTCTGAAAGCGCTACCCCTGAGTCCGGC |
| | CCAGGTACTTCTGAAAGCGCTACTCCTGAATCCGGTCCAGGTACTTCTGAAAGCGCTA |
| | CCCCGGAATCTGGCCCAGGTAGCGAACCGGCTACTTCTGGTTCTGAAACCCCAGGTAG |
| | CGAACCGGCTACCTCCGGTTCTGAAACTCCAGGTAGCCCAGCAGGCTCTCCGACTTCC |
| | ACTGAGGAAGGTACTTCTACTGAACCTTCCGAAGGCAGCGCACCAGGTACCTCTACTG |
| | AACCTTCTGAGGGCAGCGCTCCAGGTAGCGAACCTGCAACCTCTGGCTCTGAAACCCC |
| | AGGTACCTCTGAAAGCGCTACTCCTGAATCTGGCCCAGGTACTTCTACTGAACCGTCC |
| | GAGGGCAGCGCACCA |
| AM1318 | GGTACTTCTACTGAACCGTCTGAAGGCAGCGCACCAGGTAGCGAACCGGCTACTTCCG |
| | GTTCTGAAACCCCAGGTAGCCCAGCAGGTTCTCCAACTTCTACTGAAGAAGGTTCTAC |
| | CAGCTCTACCGCAGAATCTCCTGGTCCAGGTACCTCTACTCCGGAAAGCGGCTCTGCA |
| | TCTCCAGGTTCTACTAGCGAATCTCCTTCTGGCACTGCACCAGGTTCTACTAGCGAATC |
| | CCCGTCTGGTACTGCTCCAGGTACTTCTACTCCTGAAAGCGGTTCCGCTTCTCCAGGTA |
| | CCTCTACTCCGGAAAGCGGTTCTGCATCTCCAGGTAGCGAACCGGCAACCTCCGGCTC |
| | TGAAACCCCAGGTACCTCTGAAAGCGCTACTCCTGAATCCGGCCCAGGTAGCCCGGCA |
| | GGTTCTCCGACTTCCACTGAGGAAGGTACCTCTACTGAACCTTCTGAGGGCAGCGCTC |
| | CAGGTACTTCTGAAAGCGCTACCCCGGAGTCCGGTCCAGGTACTTCTACTGAACCGTC |
| | CGAAGGTAGCGCACCAGGTACTTCTACCGAACCGTCCGAGGGTAGCGCACCAGGTAG |
| | CCCAGCAGGTTCTCCTACCTCCACCGAGGAAGGTACTTCTACCGAACCGTCCGAGGGT |
| | AGCGCACCAGGTACTTCTACCGAACCTTCCGAGGGCAGCGCACCAGGTACTTCTGAAA |
| | GCGCTACCCCTGAGTCCGGCCCAGGTACTTCTGAAAGCGCTACTCCTGAATCCGGTCC |
| | AGGTACCTCTACTGAACCTTCCGAAGGCAGCGCTCCAGGTACCTCTACCGAACCGTCC |
| | GAGGGCAGCGCACCAGGTACTTCTGAAAGCGCAACCCCTGAATCCGGTCCAGGTACTT |
| | CTACTGAACCTTCCGAAGGTAGCGCTCCAGGTAGCGAACCTGCTACTTCTGGTTCTGA |
| | AACCCCAGGTAGCCCGGCTGGCTCTCCGACCTCCACCGAGGAAGGTAGCTCTACCCCG |
| | TCTGGTGCTACTGGTTCTCCAGGTACTCCGGGCAGCGGTACTGCTTCTTCCTCTCCAGG |
| | TAGCTCTACCCCTTCTGGTGCTACTGGCTCTCCAGGTACCTCTACCGAACCGTCCGAGG |
| | GTAGCGCACCAGGTACCTCTACTGAACCGTCTGAGGGTAGCGCTCCAGGTAGCGAACC |
| | GGCAACCTCCGGTTCTGAAACTCCAGGTAGCCCTGCTGGCTCTCCGACTTCTACTGAG |
| | GAAGGTAGCCCGGCTGGTTCTCCGACTTCTACTGAGGAAGGTACTTCTACCGAACCTT |
| | CCGAAGGTAGCGCTCCAGGTCCAGAACCAACGGGGCCGGCCCCAAGCGGAGGTAGCG |
| | AACCGGCAACCTCCGGCTCTGAAACCCCAGGTACCTCTGAAAGCGCTACTCCTGAATC |
| | CGGCCCAGGTAGCCCGGCAGGTTCTCCGACTTCCACTGAGGAAGGTACTTCTGAAAGC |
| | GCTACTCCTGAGTCCGGCCCAGGTAGCCCGGCTGGCTCTCCGACTTCCACCGAGGAAG |
| | GTAGCCCGGCTGGCTCTCCAACTTCTACTGAAGAAGGTACTTCTGAAAGCGCTACTCC |
| | TGAGTCCGGCCCAGGTAGCCCGGCTGGCTCTCCGACTTCCACCGAGGAAGGTAGCCCG |
| | GCTGGCTCTCCAACTTCTACTGAAGAAGGTTCTACCAGCTCTACCGCTGAATCTCCTGG |
| | CCCAGGTTCTACTAGCGAATCTCCGTCTGGCACCGCACCAGGTACTTCCCCTAGCGGT |
| | GAATCTTCTACTGCACCAGGTTCTACCAGCGAATCTCCTTCTGGCACCGCTCCAGGTTC |
| | TACTAGCGAATCCCCGTCTGGTACCGCACCAGGTACTTCTCCTAGCGGCGAATCTTCTA |
| | CCGCACCAGGTACTTCTACCGAACCTTCCGAGGGCAGCGCACCAGGTACTTCTGAAAG |
| | CGCTACCCCTGAGTCCGGCCCAGGTACTTCTGAAAGCGCTACTCCTGAATCCGGTCCA |
| | GGTAGCGAACCGGCAACCTCTGGCTCTGAAACCCCAGGTACCTCTGAAAGCGCTACTC |
| | CGGAATCTGGTCCAGGTACTTCTGAAAGCGCTACTCCGGAATCCGGTCCAGGTACCTC |
| | TACTGAACCTTCTGAGGGCAGCGCTCCAGGTACTTCTGAAAGCGCTACCCCGGAGTCC |
| | GGTCCAGGTACTTCTACTGAACCGTCCGAAGGTAGCGCACCAGGTACCTCCCCTAGCG |
| | GCGAATCTTCTACTGCTCCAGGTACCTCTCCTAGCGGCGAATCTTCTACCGCTCCAGGT |
| | ACCTCCCCTAGCGGTGAATCTTCTACCGCACCAGGTACTTCTACCGAACCGTCCGAGG |
| | GTAGCGCACCAGGTAGCCCAGCAGGTTCTCCTACCTCCACCGAGGAAGGTACTTCTAC |
| | CGAACCGTCCGAGGGTAGCGCACCAGGTTCTAGCCCTTCTGCTTCCACCGGTACCGGC |
| | CCAGGTAGCTCTACTCCGTCTGGTGCAACTGGCTCTCCAGGTAGCTCTACTCCGTCTGG |
| | TGCAACCGGCTCCCCAGGTAGCTCTACCCCGTCTGGTGCTACCGGCTCTCCAGGTAGC |
| | TCTACCCCGTCTGGTGCAACCGGCTCCCCAGGTGCATCCCCGGGTACTAGCTCTACCG |
| | GTTCTCCAGGTGCAAGCGCAAGCGGCGCGCCAAGCACGGGAGGTACTTCTCCGAGCG |
| | GTGAATCTTCTACCGCACCAGGTTCTACTAGCTCTACCGCTGAATCTCCGGGCCCAGGT |
| | ACTTCTCCGAGCGGTGAATCTTCTACTGCTCCAGGTACCTCTGAAAGCGCTACTCCGG |
| | AGTCTGGCCCAGGTACCTCTACTGAACCGTCTGAGGGTAGCGCTCCAGGTACTTCTAC |
| | TGAACCGTCCGAAGGTAGCGCACCAGGTTCTAGCCCTTCTGCATCTACTGGTACTGGC |
| | CCAGGTAGCTCTACTCCTTCTGGTGCTACCGGCTCTCCAGGTGCTTCTCCGGGTACTAG |
| | CTCTACCGGTTCTCCAGGTACTTCTACTCCGGAAAGCGGTTCCGCATCTCCAGGTACTT |
| | CTCCTAGCGGTGAATCTTCTACTGCTCCAGGTACCTCTCCTAGCGGCGAATCTTCTACT |
| | GCTCCAGGTACTTCTGAAAGCGCAACCCCTGAATCCGGTCCAGGTAGCGAACCGGCTA |
| | CTTCTGGCTCTGAGACTCCAGGTACTTCTACCGAACCGTCCGAAGGTAGCGCACCAGG |
| | TTCTACCAGCGAATCCCCTTCTGGTACTGCTCCAGGTTCTACCAGCGAATCCCCTTCTG |
| | GCACCGCACCAGGTACTTCTACCCCTGAAAGCGGCTCCGCTTCTCCAGGTAGCCCGGC |
| | AGGCTCTCCGACCTCTACTGAGGAAGGTACTTCTGAAAGCGCAACCCCGGAGTCCGGC |
| | CCAGGTACCTCTACCGAACCGTCTGAGGGCAGCGCACCAGGTAGCCCTGCTGGCTCTC |
| | CAACCTCCACCGAAGAAGGTACCTCTGAAAGCGCAACCCCTGAATCCGGCCCAGGTA |
| | GCGAACCGGCAACCTCCGGTTCTGAAACCCCAGGTAGCTCTACCCCGTCTGGTGCTAC |
| | CGGTTCCCCAGGTGCTTCTCCTGGTACTAGCTCTACCGGTTCTCCAGGTAGCTCTACCC |
| | CGTCTGGTGCTACTGGCTCTCCAGGTTCTACTAGCGAATCCCCGTCTGGTACTGCTCCA |
| | GGTACTTCCCCTAGCGGTGAATCTTCTACTGCTCCAGGTTCTACCAGCTCTACCGCAGA |
| | ATCTCCGGGTCCAGGTAGCTCTACCCCTTCTGGTGCAACCGGCTCTCCAGGTGCATCCC |
| | CGGGTACCAGCTCTACCGGTTCTCCAGGTACTCCGGGTAGCGGTACCGCTTCTTCCTCT |
| | CCAGGTAGCCCTGCTGGCTCTCCGACTTCTACTGAGGAAGGTAGCCCGGCTGGTTCTC |
| | CGACTTCTACTGAGGAAGGTACTTCTACCGAACCTTCCGAAGGTAGCGCTCCA |
| BC864 | GGTACTTCCACCGAACCATCCGAACCAGGTAGCGCAGGTACTTCCACCGAACCATCCG |
| | AACCTGGCAGCGCAGGTAGCGAACCGGCAACCTCTGGTACTGAACCATCAGGTAGCG |
| | GCGCATCCGAGCCTACCTCTACTGAACCAGGTAGCGAACCGGCTACCTCCGGTACTGA |
| | GCCATCAGGTAGCGAACCGGCAACTTCCGGTACTGAACCATCAGGTAGCGAACCGGC |
| | AACTTCCGGCACTGAACCATCAGGTAGCGGTGCATCTGAGCCGACCTCTACTGAACCA |
| | GGTACTTCTACTGAACCATCTGAGCCGGGCAGCGCAGGTAGCGAACCAGCTACTTCTG |
| | GCACTGAACCATCAGGTACTTCTACTGAACCATCCGAACCAGGTAGCGCAGGTAGCGA |
| | ACCTGCTACCTCTGGTACTGAGCCATCAGGTAGCGAACCGGCTACCTCTGGTACTGAA |
| | CCATCAGGTACTTCTACCGAACCATCCGAGCCTGGTAGCGCAGGTACTTCTACCGAAC |
| | CATCCGAGCCAGGCAGCGCAGGTAGCGAACCGGCAACCTCTGGCACTGAGCCATCAG |
| | GTAGCGAACCAGCAACTTCTGGTACTGAACCATCAGGTACTAGCGAGCCATCTACTTC |
| | CGAACCAGGTGCAGGTAGCGGCGCATCCGAACCTACTTCCACTGAACCAGGTACTAGC |
| | GAGCCATCCACCTCTGAACCAGGTGCAGGTAGCGAACCGGCAACTTCCGGCACTGAA |
| | CCATCAGGTAGCGAACCGGCTACCTCTGGTACTGAACCATCAGGTACTTCTACCGAAC |
| | CATCCGAGCCTGGTAGCGCAGGTACTTCTACCGAACCATCCGAGCCAGGCAGCGCAG |
| | GTAGCGGTGCATCCGAGCCGACCTCTACTGAACCAGGTAGCGAACCAGCAACTTCTGG |
| | CACTGAGCCATCAGGTAGCGAACCAGCTACCTCTGGTACTGAACCATCAGGTAGCGAA |
| | CCGGCTACTTCCGGCACTGAACCATCAGGTAGCGAACCAGCAACCTCCGGTACTGAAC |
| | CATCAGGTACTTCCACTGAACCATCCGAACCGGGTAGCGCAGGTAGCGAACCGGCAA |
| | CTTCCGGCACTGAACCATCAGGTAGCGGTGCATCTGAGCCGACCTCTACTGAACCAGG |
| | TACTTCTACTGAACCATCTGAGCCGGGCAGCGCAGGTAGCGAACCTGCAACCTCCGGC |
| | ACTGAGCCATCAGGTAGCGGCGCATCTGAACCAACCTCTACTGAACCAGGTACTTCCA |
| | CCGAACCATCTGAGCCAGGCAGCGCAGGTAGCGGCGCATCTGAACCAACCTCTACTG |
| | AACCAGGTAGCGAACCAGCAACTTCTGGTACTGAACCATCAGGTAGCGGCGCATCTG |
| | AGCCTACTTCCACTGAACCAGGTAGCGAACCGGCAACTTCCGGCACTGAACCATCAGG |
| | TAGCGGTGCATCTGAGCCGACCTCTACTGAACCAGGTACTTCTACTGAACCATCTGAG |
| | CCGGGCAGCGCAGGTAGCGAACCGGCAACTTCCGGCACTGAACCATCAGGTAGCGGT |
| | GCATCTGAGCCGACCTCTACTGAACCAGGTACTTCTACTGAACCATCTGAGCCGGGCA |
| | GCGCAGGTAGCGAACCAGCTACTTCTGGCACTGAACCATCAGGTACTTCTACTGAACC |
| | ATCCGAACCAGGTAGCGCAGGTAGCGAACCTGCTACCTCTGGTACTGAGCCATCAGGT |
| | ACTTCTACTGAACCATCCGAGCCGGGTAGCGCAGGTACTTCCACTGAACCATCTGAAC |
| | CTGGTAGCGCAGGTACTTCCACTGAACCATCCGAACCAGGTAGCGCAGGTACTTCTAC |
| | TGAACCATCCGAGCCGGGTAGCGCAGGTACTTCCACTGAACCATCTGAACCTGGTAGC |
| | GCAGGTACTTCCACTGAACCATCCGAACCAGGTAGCGCAGGTACTAGCGAACCATCCA |
| | CCTCCGAACCAGGCGCAGGTAGCGGTGCATCTGAACCGACTTCTACTGAACCAGGTAC |
| | TTCCACTGAACCATCTGAGCCAGGTAGCGCAGGTACTTCCACCGAACCATCCGAACCA |
| | GGTAGCGCAGGTACTTCCACCGAACCATCCGAACCTGGCAGCGCAGGTAGCGAACCG |
| | GCAACCTCTGGTACTGAACCATCAGGTAGCGGTGCATCCGAGCCGACCTCTACTGAAC |
| | CAGGTAGCGAACCAGCAACTTCTGGCACTGAGCCATCAGGTAGCGAACCAGCTACCTC |
| | TGGTACTGAACCATCAGGTAGCGAACCGGCAACCTCTGGCACTGAGCCATCAGGTAGC |
| | GAACCAGCAACTTCTGGTACTGAACCATCAGGTACTAGCGAGCCATCTACTTCCGAAC |
| | CAGGTGCAGGTAGCGAACCTGCAACCTCCGGCACTGAGCCATCAGGTAGCGGCGCAT |
| | CTGAACCAACCTCTACTGAACCAGGTACTTCCACCGAACCATCTGAGCCAGGCAGCGC |
| | AGGTAGCGAACCTGCAACCTCCGGCACTGAGCCATCAGGTAGCGGCGCATCTGAACC |
| | AACCTCTACTGAACCAGGTACTTCCACCGAACCATCTGAGCCAGGCAGCGCA |
| BD864 | GGTAGCGAAACTGCTACTTCCGGCTCTGAGACTGCAGGTACTAGTGAATCCGCAACTA |
| | GCGAATCTGGCGCAGGTAGCACTGCAGGCTCTGAGACTTCCACTGAAGCAGGTACTAG |
| | CGAGTCCGCAACCAGCGAATCCGGCGCAGGTAGCGAAACTGCTACCTCTGGCTCCGA |
| | GACTGCAGGTAGCGAAACTGCAACCTCTGGCTCTGAAACTGCAGGTACTTCCACTGAA |
| | GCAAGTGAAGGCTCCGCATCAGGTACTTCCACCGAAGCAAGCGAAGGCTCCGCATCA |
| | GGTACTAGTGAGTCCGCAACTAGCGAATCCGGTGCAGGTAGCGAAACCGCTACCTCTG |
| | GTTCCGAAACTGCAGGTACTTCTACCGAGGCTAGCGAAGGTTCTGCATCAGGTAGCAC |
| | TGCTGGTTCCGAGACTTCTACTGAAGCAGGTACTAGCGAATCTGCTACTAGCGAATCC |
| | GGCGCAGGTACTAGCGAATCCGCTACCAGCGAATCCGGCGCAGGTAGCGAAACTGCA |
| | ACCTCTGGTTCCGAGACTGCAGGTACTAGCGAGTCCGCTACTAGCGAATCTGGCGCAG |
| | GTACTTCCACTGAAGCTAGTGAAGGTTCTGCATCAGGTAGCGAAACTGCTACTTCTGG |
| | TTCCGAAACTGCAGGTAGCGAAACCGCTACCTCTGGTTCCGAAACTGCAGGTACTTCT |
| | ACCGAGGCTAGCGAAGGTTCTGCATCAGGTAGCACTGCTGGTTCCGAGACTTCTACTG |
| | AAGCAGGTACTAGCGAGTCCGCTACTAGCGAATCTGGCGCAGGTACTTCCACTGAAGC |
| | TAGTGAAGGTTCTGCATCAGGTAGCGAAACTGCTACTTCTGGTTCCGAAACTGCAGGT |
| | AGCACTGCTGGCTCCGAGACTTCTACCGAAGCAGGTAGCACTGCAGGTTCCGAAACTT |
| | CCACTGAAGCAGGTAGCGAAACTGCTACCTCTGGCTCTGAGACTGCAGGTACTAGCGA |
| | ATCTGCTACTAGCGAATCCGGCGCAGGTACTAGCGAATCCGCTACCAGCGAATCCGGC |
| | GCAGGTAGCGAAACTGCAACCTCTGGTTCCGAGACTGCAGGTACTAGCGAATCTGCTA |
| | CTAGCGAATCCGGCGCAGGTACTAGCGAATCCGCTACCAGCGAATCCGGCGCAGGTA |
| | GCGAAACTGCAACCTCTGGTTCCGAGACTGCAGGTAGCGAAACCGCTACCTCTGGTTC |
| | CGAAACTGCAGGTACTTCTACCGAGGCTAGCGAAGGTTCTGCATCAGGTAGCACTGCT |
| | GGTTCCGAGACTTCTACTGAAGCAGGTAGCGAAACTGCTACTTCCGGCTCTGAGACTG |
| | CAGGTACTAGTGAATCCGCAACTAGCGAATCTGGCGCAGGTAGCACTGCAGGCTCTGA |
| | GACTTCCACTGAAGCAGGTAGCACTGCTGGTTCCGAAACCTCTACCGAAGCAGGTAGC |
| | ACTGCAGGTTCTGAAACCTCCACTGAAGCAGGTACTTCCACTGAGGCTAGTGAAGGCT |
| | CTGCATCAGGTAGCACTGCTGGTTCCGAAACCTCTACCGAAGCAGGTAGCACTGCAGG |
| | TTCTGAAACCTCCACTGAAGCAGGTACTTCCACTGAGGCTAGTGAAGGCTCTGCATCA |
| | GGTAGCACTGCAGGTTCTGAGACTTCCACCGAAGCAGGTAGCGAAACTGCTACTTCTG |
| | GTTCCGAAACTGCAGGTACTTCCACTGAAGCTAGTGAAGGTTCCGCATCAGGTACTAG |
| | TGAGTCCGCAACCAGCGAATCCGGCGCAGGTAGCGAAACCGCAACCTCCGGTTCTGA |
| | AACTGCAGGTACTAGCGAATCCGCAACCAGCGAATCTGGCGCAGGTACTAGTGAGTC |
| | CGCAACCAGCGAATCCGGCGCAGGTAGCGAAACCGCAACCTCCGGTTCTGAAACTGC |
| | AGGTACTAGCGAATCCGCAACCAGCGAATCTGGCGCAGGTAGCGAAACTGCTACTTCC |
| | GGCTCTGAGACTGCAGGTACTTCCACCGAAGCAAGCGAAGGTTCCGCATCAGGTACTT |
| | CCACCGAGGCTAGTGAAGGCTCTGCATCAGGTAGCACTGCTGGCTCCGAGACTTCTAC |
| | CGAAGCAGGTAGCACTGCAGGTTCCGAAACTTCCACTGAAGCAGGTAGCGAAACTGC |
| | TACCTCTGGCTCTGAGACTGCAGGTACTAGCGAATCTGCTACTAGCGAATCCGGCGCA |
| | GGTACTAGCGAATCCGCTACCAGCGAATCCGGCGCAGGTAGCGAAACTGCAACCTCT |
| | GGTTCCGAGACTGCAGGTAGCGAAACTGCTACTTCCGGCTCCGAGACTGCAGGTAGCG |
| | AAACTGCTACTTCTGGCTCCGAAACTGCAGGTACTTCTACTGAGGCTAGTGAAGGTTC |
| | CGCATCAGGTACTAGCGAGTCCGCAACCAGCGAATCCGGCGCAGGTAGCGAAACTGC |
| | TACCTCTGGCTCCGAGACTGCAGGTAGCGAAACTGCAACCTCTGGCTCTGAAACTGCA |
| | GGTACTAGCGAATCTGCTACTAGCGAATCCGGCGCAGGTACTAGCGAATCCGCTACCA |
| | GCGAATCCGGCGCAGGTAGCGAAACTGCAACCTCTGGTTCCGAGACTGCA |

*These and other exemplary sequences embody the desired features disclosed herein, including without limitation, substantially non-repetitiveness, low immunogenicity, unstructured conformation, conformational flexibility, enhanced aqueous solubility, high degree of protease resistance, low binding to mammalian receptors, a defined degree of charge, and increased hydrodynamic (or Stokes) radii.

One may clone the library of XTEN-encoding genes into one or more expression vectors known in the art. To facilitate the identification of well-expressing library members, one can construct the library as fusion to a reporter protein. Non-limiting examples of suitable reporter genes are green fluorescent protein, luciferace, alkaline phosphatase, and beta-galactosidase. By screening, one can identify short XTEN sequences that can be expressed in high concentration in the host organism of choice. Subsequently, one can generate a library of random XTEN dimers and repeat the screen for high level of expression. Subsequently, one can screen the resulting constructs for a number of properties such as level of expression, protease stability, or binding to antiserum.

One aspect of the invention is to provide polynucleotide sequences encoding the components of the fusion protein wherein the creation of the sequence has undergone codon optimization. Of particular interest is codon optimization with the goal of improving expression of the polypeptide compositions and to improve the genetic stability of the encoding gene in the production hosts. For example, codon optimization is of particular importance for XTEN sequences that are rich in glycine or that have very repetitive amino acid sequences. Codon optimization is performed using computer programs (Gustafsson, C., et al. (2004) Trends Biotechnol, 22: 346-53), some of which minimize ribosomal pausing (Coda Genomics Inc.). In one embodiment, one can perform codon optimization by constructing codon libraries where all members of the library encode the same amino acid sequence but where codon usage is varied. Such libraries can be screened for highly expressing and genetically stable members that are particularly suitable for the large-scale production of XTEN-containing products. When designing XTEN sequences one can consider a number of properties. One can minimize the repetitiveness in the encoding DNA sequences. In addition, one can avoid or minimize the use of codons that are rarely used by the production host (e.g. the AGG and AGA arginine codons and one leucine codon in E. coli). In the case of E. coli, two glycine codons, GGA and GGG, are rarely used in highly expressed proteins. Thus codon optimization of the gene encoding XTEN sequences can be very desirable. DNA sequences that have a high level of glycine tend to have a high GC content that can lead to instability or low expression levels. Thus, when possible, it is preferred to choose codons such that the GC-content of XTEN-encoding sequence is suitable for the production organism that will be used to manufacture the XTEN.

Optionally, the full-length XTEN-encoding gene comprises one or more sequencing islands. In this context, sequencing islands are short-stretch sequences that are distinct from the XTEN library construct sequences and that include a restriction site not present or expected to be present in the full-length XTEN-encoding gene. In one embodiment, a sequencing island is the sequence 5′-AGGTGCAAGCGCAAGCGGCGCGCCAAGCACGGGAGGT-3′. In another embodiment, a sequencing island is the sequence 5′-AGGTCCAGAACCAACGGGGCCGGCCCCAAGCGGAGGT-3′.

In one embodiment, polynucleotide libraries are constructed using the disclosed methods wherein all members of the library encode the same amino acid sequence but where codon usage for the respective amino acids in the sequence is varied. Such libraries can be screened for highly expressing and genetically stable members that are particularly suitable for the large-scale production of XTEN-containing products.

Optionally, one can sequence clones in the library to eliminate isolates that contain undesirable sequences. The initial library of short XTEN sequences allows some variation in amino acid sequence. For instance one can randomize some codons such that a number of hydrophilic amino acids can occur in a particular position. During the process of iterative multimerization one can screen the resulting library members for other characteristics like solubility or protease resistance in addition to a screen for high-level expression.

Once the gene that encodes the XTEN of desired length and properties is selected, it is genetically fused at the desired location to the nucleotides encoding the FVIII gene(s) by cloning it into the construct adjacent and in frame with the gene coding for CF, or alternatively between nucleotides encoding adjacent domains of the CF, or alternatively within a sequence encoding a given FVIII domain, or alternatively in frame with nucleotides encoding a spacer/cleavage sequence linked to a terminal XTEN. The invention provides various permutations of the foregoing, depending on the CFXTEN to be encoded. For example, a gene encoding a CFXTEN fusion protein comprising a FVIII and two XTEN, such as embodied by formula VI, as depicted above, the gene would have polynucleotides encoding CF, encoding two XTEN, which can be identical or different in composition and sequence length. In one non-limiting embodiment of the foregoing, the FVIII polynucleotides would encode coagulation factor and the polynucleotides encoding the C-terminus XTEN would encode AE864 and the polynucleotides encoding an internal XTEN adjacent to the C-terminus of the A2 domain would encode AE144. The step of cloning the FVIII genes into the XTEN construct can occur through a ligation or multimerization step, as shown in FIG. 12. The constructs encoding CFXTEN fusion proteins can be designed in different configurations of the components XTEN, CF, and spacer sequences, such as the configurations of formulae I-VIII. In one embodiment, the construct comprises polynucleotide sequences complementary to, or those that encode a monomeric polypeptide of components in the following order (5′ to 3′) FVIII and XTEN. In another embodiment, the construct comprises polynucleotide sequences complementary to, or those that encode a monomeric polypeptide of components in the following order (5′ to 3′) CF, spacer sequence, and XTEN. The spacer polynucleotides can optionally comprise sequences encoding cleavage sequences. As will be apparent to those of skill in the art, other permutations or multimers of the foregoing are possible.

The invention also encompasses polynucleotides comprising XTEN-encoding polynucleotide variants that have a high percentage of sequence identity compared to (a) a polynucleotide sequence from Table 8, or (b) sequences that are complementary to the polynucleotides of (a). A polynucleotide with a high percentage of sequence identity is one that has at least about an 80% nucleic acid sequence identity, alternatively at least about 81%, alternatively at least about 82%, alternatively at least about 83%, alternatively at least about 84%, alternatively at least about 85%, alternatively at least about 86%, alternatively at least about 87%, alternatively at least about 88%, alternatively at least about 89%, alternatively at least about 90%, alternatively at least about 91%, alternatively at least about 92%, alternatively at least about 93%, alternatively at least about 94%, alternatively at least about 95%, alternatively at least about 96%, alternatively at least about 97%, alternatively at least about 98%, and alternatively at least about 99% nucleic acid sequence identity compared to (a) or (b) of the foregoing, or that can hybridize with the target polynucleotide or its complement under stringent conditions.

Homology, sequence similarity or sequence identity of nucleotide or amino acid sequences may also be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman (Advances in Applied Mathematics. 1981. 2: 482-489), to find the best segment of identity or similarity between two sequences. Gap performs global alignments: all of one sequence with all of another similar sequence using the method of Needleman and Wunsch, (Journal of Molecular Biology. 1970. 48:443-453). When using a sequence alignment program such as BestFit, to determine the degree of sequence homology, similarity or identity, the default setting may be used, or an appropriate scoring matrix may be selected to optimize identity, similarity or homology scores.

Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the polynucleotides that encode the CFXTEN sequences under stringent conditions, such as those described herein.

The resulting polynucleotides encoding the CFXTEN chimeric fusion proteins can then be individually cloned into an expression vector. The nucleic acid sequence is inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature.

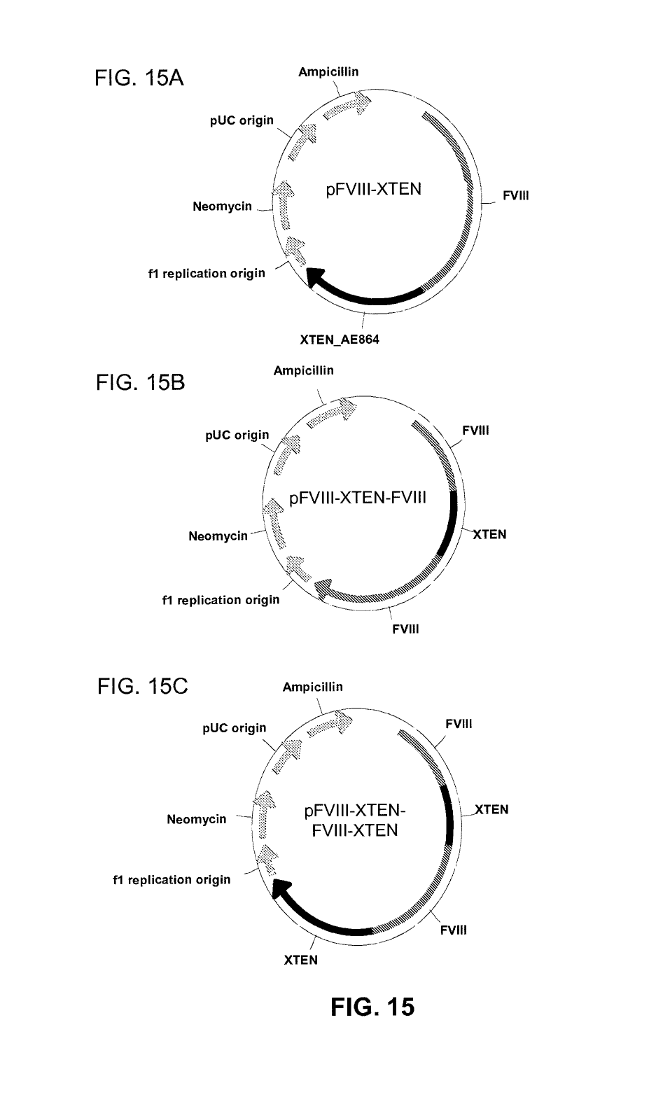

Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Representative plasmids are illustrated in FIG. 15, with encoding regions for different configurations of FVIII and XTEN components portrayed.

The invention provides for the use of plasmid vectors containing replication and control sequences that are compatible with and recognized by the host cell, and are operably linked to the CFXTEN gene for controlled expression of the CFXTEN fusion proteins. The vector ordinarily carries a replication site, as well as sequences that encode proteins that are capable of providing phenotypic selection in transformed cells. Such vector sequences are well known for a variety of bacteria, yeast, and viruses. Useful expression vectors that can be used include, for example, segments of chromosomal, non-chromosomal and synthetic DNA sequences. “Expression vector” refers to a DNA construct containing a DNA sequence that is operably linked to a suitable control sequence capable of effecting the expression of the DNA encoding the fusion protein in a suitable host. The requirements are that the vectors are replicable and viable in the host cell of choice. Low- or high-copy number vectors may be used as desired.

Other suitable vectors include, but are not limited to, derivatives of SV40 and pcDNA and known bacterial plasmids such as col EI, pCR1, pBR322, pMal-C2, pET, pGEX as described by Smith, et al., Gene 57:31-40 (1988), pMB9 and derivatives thereof, plasmids such as RP4, phage DNAs such as the numerous derivatives of phage I such as NM98 9, as well as other phage DNA such as M13 and filamentous single stranded phage DNA; yeast plasmids such as the 2 micron plasmid or derivatives of the 2 m plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like. Yeast expression systems that can also be used in the present invention include, but are not limited to, the non-fusion pYES2 vector (Invitrogen), the fusion pYESHisA, B, C (Invitrogen), pRS vectors and the like.

The control sequences of the vector include a promoter to effect transcription, an optional operator sequence to control such transcription, a sequence encoding suitable mRNA ribosome binding sites, and sequences that control termination of transcription and translation. The promoter may be any DNA sequence, which shows transcriptional activity in the host cell of choice and may be derived from genes encoding proteins either homologous or heterologous to the host cell.

Examples of suitable promoters for directing the transcription of the DNA encoding the FVIII polypeptide variant in mammalian cells are the SV40 promoter (Subramani et al., Mol. Cell. Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982). The vector may also carry sequences such as UCOE (ubiquitous chromatin opening elements).

Examples of suitable promoters for use in filamentous fungus host cells are, for instance, the ADH3 promoter or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamoriglucoamylase (gluA), Rhizomucor miehei lipase, A. oryzae alkaline protease, A. oryzae triose phosphate isomerase or A. nidulans acetamidase. Preferred are the TAKA-amylase and gluA promoters.

Promoters suitable for use in expression vectors with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)], all is operably linked to the DNA encoding CFXTEN polypeptides. Promoters for use in bacterial systems can also contain a Shine-Dalgarno (S.D.) sequence, operably linked to the DNA encoding CFXTEN polypeptides.

The invention contemplates use of other expression systems including, for example, a baculovirus expression system with both non-fusion transfer vectors, such as, but not limited to pVL941 Summers, et al., Virology 84:390-402 (1978)), pVL1393 (Invitrogen), pVL1392 (Summers, et al., Virology 84:390-402 (1978) and Invitrogen) and pBlueBacIII (Invitrogen), and fusion transfer vectors such as, but not limited to, pAc700 (Summers, et al., Virology 84:390-402 (1978)), pAc701 and pAc70-2 (same as pAc700, with different reading frames), pAc360 Invitrogen) and pBlueBacHisA, B, C (Invitrogen) can be used.

Examples of suitable promoters for directing the transcription of the DNA encoding the FVIII polypeptide variant in mammalian cells are the CMV promoter (Boshart et al., Cell 41:521-530, 1985), the SV40 promoter (Subramani et al., Mol. Cell. Biol. 1 (1981), 854-864), the MT-1 (metallothionein gene) promoter (Palmiter et al., Science 222 (1983), 809-814), the adenovirus 2 major late promoter (Kaufman and Sharp, Mol. Cell. Biol, 2:1304-1319, 1982). The vector may also carry sequences such as UCOE (ubiquitous chromatin opening elements).

Examples of suitable promoters for use in filamentous fungus host cells are, for instance, the ADH3 promoter or the tpiA promoter.

The DNA sequences encoding the CFXTEN may also, if necessary, be operably connected to a suitable terminator, such as the hGH terminator (Palmiter et al., Science 222, 1983, pp. 809-814) or the TPI1 terminators (Alber and Kawasaki, J. Mol. Appl. Gen. 1, 1982, pp. 419-434) or ADH3 (McKnight et al., The EMBO J. 4, 1985, pp. 2093-2099). Expression vectors may also contain a set of RNA splice sites located downstream from the promoter and upstream from the insertion site for the CFXTEN sequence itself, including splice sites obtained from adenovirus. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 E1b region, the hGH terminator (DeNoto et al. Nucl. Acids Res. 9:3719-3730, 1981). The expression vectors may also include a noncoding viral leader sequence, such as the adenovirus 2 tripartite leader, located between the promoter and the RNA splice sites; and enhancer sequences, such as the SV40 enhancer.

To direct the CFXTEN of the present invention into the secretory pathway of the host cells, a secretory signal sequence (a.k.a., a leader sequence, a prepro sequence, or a pre sequence) may be included in the recombinant vector. The secretory signal sequence is operably linked to the DNA sequences encoding the CFXTEN, usually positioned 5′ to the DNA sequence encoding the CFXTEN fusion protein. The secretory signal sequence may be that, normally associated with the protein or may be from a gene encoding another secreted protein. Non-limiting examples include OmpA, PhoA, and DsbA for E. coli expression, ppL-alpha, DEX4, invertase signal peptide, acid phosphatase signal peptide, CPY, or INU1 for yeast expression, and IL2L, SV40, IgG kappa and IgG lambda for mammalian expression. Signal sequences are typically proteolytically removed from the protein during the translocation and secretion process, generating a defined N-terminus. Methods are disclosed in Arnau, et al., Protein Expression and Purification 48: 1-13 (2006).

The procedures used to ligate the DNA sequences coding for the CFXTEN, the promoter and optionally the terminator and/or secretory signal sequence, respectively, and to insert them into suitable vectors containing the information necessary for replication, are well known to persons skilled in the art (cf., for instance, Sambrook, J. et al., “Molecular Cloning: A Laboratory Manual,” 3^{rd} edition, Cold Spring Harbor Laboratory Press, 2001).

In other cases, the invention provides constructs and methods of making constructs comprising an polynucleotide sequence optimized for expression that encodes at least about 20 to about 60 amino acids with XTEN characteristics that can be included at the N-terminus of an XTEN carrier encoding sequence (in other words, the polynucleotides encoding the 20-60 encoded optimized amino acids are linked in frame to polynucleotides encoding an XTEN component that is N-terminal to CF) to promote the initiation of translation to allow for expression of XTEN fusions at the N-terminus of proteins without the presence of a helper domain. In an advantage of the foregoing, the sequence does not require subsequent cleavage, thereby reducing the number of steps to manufacture XTEN-containing compositions. As described in more detail in the Examples, the optimized N-terminal sequence has attributes of an unstructured protein, but may include nucleotide bases encoding amino acids selected for their ability to promote initiation of translation and enhanced expression. In one embodiment of the foregoing, the optimized polynucleotide encodes an XTEN sequence with at least about 90% sequence identity compared to AE912. In another embodiment of the foregoing, the optimized polynucleotide encodes an XTEN sequence with at least about 90% sequence identity compared to AM923. In another embodiment of the foregoing, the optimized polynucleotide encodes an XTEN sequence with at least about 90% sequence identity compared to AE48. In another embodiment of the foregoing, the optimized polynucleotide encodes an XTEN sequence with at least about 90% sequence identity compared to AM48. In one embodiment, the optimized polynucleotide NTS comprises a sequence that exhibits at least about 80%, at least about 85%, at least about 90%, at least about 91%, at least about 92%, at least about 93%, at least about 94%, at least about 95%, at least about 96%, at least about 97%, at least about 98%, or at least about 99%, sequence identity compared to a sequence or its complement selected from

AE 48: 5′-
ATGGCTGAACCTGCTGGCTCTCCAACCTCCACTGAGGAAGGTACCCCGGG
TAGCGGTACTGCTTCTTCCTCTCCAGGTAGCTCTACCCCTTCTGGTGCAA
CCGGCTCTCCAGGTGCTTCTCCGGGCACCAGCTCTACCGGTTCTCCA-3′
and
AM 48: 5′-
ATGGCTGAACCTGCTGGCTCTCCAACCTCCACTGAGGAAGGTGCATCCCC
GGGCACCAGCTCTACCGGTTCTCCAGGTAGCTCTACCCCGTCTGGTGCTA
CCGGCTCTCCAGGTAGCTCTACCCCGTCTGGTGCTACTGGCTCTCCA-3′

In this manner, a chimeric DNA molecule coding for a monomeric CFXTEN fusion protein is generated within the construct. Optionally, this chimeric DNA molecule may be transferred or cloned into another construct that is a more appropriate expression vector. At this point, a host cell capable of expressing the chimeric DNA molecule can be transformed with the chimeric DNA molecule.

Non-limiting examples of mammalian cell lines for use in the present invention are the COS-1 (ATCC CRL 1650), COS-7 (ATCC CRL 1651), BHK-21 (ATCC CCL 10)) and BHK-293 (ATCC CRL 1573; Graham et al., J. Gen. Virol. 36:59-72, 1977), BHK-570 cells (ATCC CRL 10314), CHO-K1 (ATCC CCL 61), CHO-S (Invitrogen 11619-012), and 293-F (Invitrogen R790-7), and the parental and derivative cell lines known in the art useful for expression of FVIII. A tk-ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the CFXTEN may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279). Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

Other suitable cells that can be used in the present invention include, but are not limited to, prokaryotic host cells strains such as Escherichia coli, (e.g., strain DHS-α), Bacillus subtilis, Salmonella typhimurium, or strains of the genera of Pseudomonas, Streptomyces and Staphylococcus. Non-limiting examples of suitable prokaryotes include those from the genera: Actinoplanes; Archaeoglobus; Bdellovibrio; Borrelia; Chloroflexus; Enterococcus; Escherichia; Lactobacillus; Listeria; Oceanobacillus; Paracoccus; Pseudomonas; Staphylococcus; Streptococcus; Streptomyces; Thermoplasma; and Vibrio.

Methods of transfecting mammalian cells and expressing DNA sequences introduced in the cells are described in e.g., Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Cloned DNA sequences are introduced into cultured mammalian cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d:456-467, 1973), transfection with many commercially available reagents such as FuGENEG Roche Diagnostics, Mannheim, Germany) or lipofectamine (Invitrogen) or by electroporation (Neumann et al., EMBO J. 1:841-845, 1982). To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, puromycin, zeocin, and methotrexate. The selectable marker may be an amplifiable selectable marker. A preferred amplifiable selectable marker is a dihydrofolate reductase (DHFR) sequence. Further examples of selectable markers are well known to one of skill in the art and include reporters such as enhanced green fluorescent protein (EGFP), beta-galactosidase (β-gal) or chloramphenicol acetyltransferase (CAT). Selectable markers are reviewed by Thilly (Mammalian Cell Technology, Butterworth Publishers, Stoneham, Mass., incorporated herein by reference). The person skilled in the art will easily be able to choose suitable selectable markers. Any known selectable marker may be employed so long as it is capable of being expressed simultaneously with the nucleic acid encoding a gene product.

Selectable markers may be introduced into the cell on a separate plasmid at the same time as the gene of interest, or they may be introduced on the same plasmid. If, on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the CFXTEN of interest. Media generally include a carbon source, a nitrogen source, essential amino acids, essential sugars, vitamins, salts, phospholipids, protein and growth factors. For production of gamma-carboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the FVIII polypeptide variant of interest.

The transformed or transfected host cell is then cultured in a suitable nutrient medium under conditions permitting expression of the FVIII polypeptide variant after which the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The culture conditions, such as temperature, pH and the like, are those previously used with the host cell selected for expression, and will be apparent to the ordinarily skilled artisan.

Gene expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological of fluorescent methods, such as immunohistochemical staining of cells or tissue sections and assay of cell culture or body fluids or the detection of selectable markers, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence FVIII polypeptide or against a synthetic peptide based on the DNA sequences provided herein or against exogenous sequence fused to FVIII and encoding a specific antibody epitope. Examples of selectable markers are well known to one of skill in the art and include reporters such as enhanced green fluorescent protein (EGFP), beta-galactosidase (β-gal) or chloramphenicol acetyltransferase (CAT).

Expressed CFXTEN polypeptide product(s) may be purified via methods known in the art or by methods disclosed herein. Procedures such as gel filtration, affinity purification (e.g., using an anti-FVIII antibody column), salt fractionation, ion exchange chromatography, size exclusion chromatography, hydroxyapatite adsorption chromatography, hydrophobic interaction chromatography and gel electrophoresis may be used; each tailored to recover and purify the fusion protein produced by the respective host cells. Additional purification may be achieved by conventional chemical purification means, such as high performance liquid chromatography. Some expressed CFXTEN may require refolding during isolation and purification. Methods of purification are described in Robert K. Scopes, Protein Purification: Principles and Practice, Charles R. Castor (ed.), Springer-Verlag 1994, and Sambrook, et al., supra. Multi-step purification separations are also described in Baron, et al., Crit. Rev. Biotechnol. 10:179-90 (1990) and Below, et al., J. Chromatogr. A. 679:67-83 (1994). For therapeutic purposes it is preferred that the CFXTEN fusion proteins of the invention are substantially pure. Thus, in a preferred embodiment of the invention the CFXTEN of the invention is purified to at least about 90 to 95% homogeneity, preferably to at least about 98% homogeneity. Purity may be assessed by, e.g., gel electrophoresis, HPLC, and amino-terminal amino acid sequencing.

### VIII). Pharmaceutical Compositions

The present invention provides pharmaceutical compositions comprising CFXTEN. In one embodiment, the pharmaceutical composition comprises a CFXTEN fusion protein disclosed herein and at least one pharmaceutically acceptable carrier. CFXTEN polypeptides of the present invention can be formulated according to known methods to prepare pharmaceutically useful compositions, whereby the polypeptide is combined in admixture with a pharmaceutically acceptable carrier vehicle, such as aqueous solutions, buffers, solvents and/or pharmaceutically acceptable suspensions, emulsions, stabilizers or excipients. Examples of non-aqueous solvents include propyl ethylene glycol, polyethylene glycol and vegetable oils. Formulations of the pharmaceutical compositions are prepared for storage by mixing the active CFXTEN ingredient having the desired degree of purity with optional physiologically acceptable carriers, excipients (e.g., sodium chloride, a calcium salt, sucrose, or polysorbate) or stabilizers (e.g., sucrose, trehalose, raffinose, arginine, a calcium salt, glycine or histidine), as described in Remington's Pharmaceutical Sciences 16th edition, Osol, A. Ed. (1980), in the form of lyophilized formulations or aqueous solutions.

In one embodiment, the pharmaceutical composition may be supplied as a lyophilized powder to be reconstituted prior to administration. In another embodiment, the pharmaceutical composition may be supplied in a liquid form, which can be administered directly to a patient. In another embodiment, the composition is supplied as a liquid in a pre-filled syringe for administration of the composition. In another embodiment, the composition is supplied as a liquid in a pre-filled vial that can be incorporated into a pump.

The pharmaceutical compositions can be administered by any suitable means or route, including subcutaneously, subcutaneously by infusion pump, intramuscularly, and intravenously. It will be appreciated that the preferred route will vary with the disease and age of the recipient, and the severity of the condition being treated.

In one embodiment, the CFXTEN pharmaceutical composition in liquid form or after reconstitution (when supplied as a lyophilized powder) comprises coagulation factor VIII with an activity of at least 50 IU/ml, or at least 100 IU/ml, or at least 200 IU/ml, or at least 300 IU/ml, or at least 400 IU/ml, or an activity of at least 500 IU/ml, or an activity of at least 600 IU/ml, which composition is capable of increasing factor VIII activity to at least 1.5% of the normal plasma level in the blood for at least about 12 hours, or at least about 24 hours, or at least about 48 hours, or at least about 72 hours, or at least about 96 hours, or at least about 120 hours after administration of the factor VIII pharmaceutical composition to a subject in need of routine prophylaxis. In another embodiment, the CFXTEN pharmaceutical composition in liquid form or after reconstitution (when supplied as a lyophilized powder) comprises coagulation factor VIII with an activity of at least 50 IU/ml, or at least 100 IU/ml, or at least 200 IU/ml, or at least 300 IU/ml, or at least 400 IU/ml, or at least 500 IU/ml, or an activity of at least 600 IU/ml, which composition is capable of increasing factor VIII activity to at least 2.5% of the normal plasma level in the blood for at least about 12 hours, or at least about 24 hours, or at least about 48 hours, or at least about 72 hours, or at least about 96 hours, or at least about 120 hours after administration to a subject in need of routine prophylaxis. It is specifically contemplated that the pharmaceutical compositions of the foregoing can be formulated to include one or more excipients, buffers or other ingredients known in the art to be compatible with administration by the intravenous route or the subcutaneous route or the intramuscular route. Thus, in the embodiments hereinabove described in this paragraph, the pharmaceutical composition is administered subcutaneously, intramuscularly or intravenously.

The compositions of the invention may be formulated using a variety of excipients. Suitable excipients include microcrystalline cellulose (e.g. Avicel PH102, Avicel PH101), polymethacrylate, poly(ethyl acrylate, methyl methacrylate, trimethylammonioethyl methacrylate chloride) (such as Eudragit RS-30D), hydroxypropyl methylcellulose (Methocel K100M, Premium CR Methocel K100M, Methocel E5, Opadry®), magnesium stearate, talc, triethyl citrate, aqueous ethylcellulose dispersion (Surelease®), and protamine sulfate. The slow release agent may also comprise a carrier, which can comprise, for example, solvents, dispersion media, coatings, antibacterial and antifungal agents, isotonic and absorption delaying agents. Pharmaceutically acceptable salts can also be used in these slow release agents, for example, mineral salts such as hydrochlorides, hydrobromides, phosphates, or sulfates, as well as the salts of organic acids such as acetates, proprionates, malonates, or benzoates. The composition may also contain liquids, such as water, saline, glycerol, and ethanol, as well as substances such as wetting agents, emulsifying agents, or pH buffering agents. Liposomes may also be used as a carrier.

In another embodiment, the compositions of the present invention are encapsulated in liposomes, which have demonstrated utility in delivering beneficial active agents in a controlled manner over prolonged periods of time. Liposomes are closed bilayer membranes containing an entrapped aqueous volume. Liposomes may also be unilamellar vesicles possessing a single membrane bilayer or multilamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. In one embodiment, the liposome may be coated with a flexible water soluble polymer that avoids uptake by the organs of the mononuclear phagocyte system, primarily the liver and spleen. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose hydroxyethylcellulose, polyethyleneglycol, polyaspartamide and hydrophilic peptide sequences as described in U.S. Pat. Nos. 6,316,024; 6,126,966; 6,056,973; 6,043,094, the contents of which are incorporated by reference in their entirety.

Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phasphatidylglycerol, phosphatidylinositol, and sphingomyelin as disclosed in U.S. Pat. Nos. 6,056,973 and 5,874,104. The vesicle-forming lipids may also be glycolipids, cerebrosides, or cationic lipids, such as 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1[(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3 [N—(N′,N′-dimethylaminoethane) carbamoly] cholesterol (DC-Chol); or dimethyldioctadecylammonium (DDAB) also as disclosed in U.S. Pat. No. 6,056,973. Cholesterol may also be present in the proper range to impart stability to the vesicle as disclosed in U.S. Pat. Nos. 5,916,588 and 5,874,104.

Additional liposomal technologies are described in U.S. Pat. Nos. 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479, the contents of which are incorporated herein by reference. These describe liposomes and lipid-coated microbubbles, and methods for their manufacture. Thus, one skilled in the art, considering both the disclosure of this invention and the disclosures of these other patents could produce a liposome for the extended release of the polypeptides of the present invention.

For liquid formulations, a desired property is that the formulation be supplied in a form that can pass through a 25, 28, 30, 31, 32 gauge needle for intravenous, intramuscular, intraarticular, or subcutaneous administration.

Osmotic pumps may be used as slow release agents in the form of tablets, pills, capsules or implantable devices. Osmotic pumps are well known in the art and readily available to one of ordinary skill in the art from companies experienced in providing osmotic pumps for extended release drug delivery. Examples are ALZA's DUROS™; ALZA's OROS™; Osmotica Pharmaceutical's Osmodex™ system; Shire Laboratories' EnSoTrol™ system; and Alzet™. Patents that describe osmotic pump technology are U.S. Pat. Nos. 6,890,918; 6,838,093; 6,814,979; 6,713,086; 6,534,090; 6,514,532; 6,361,796; 6,352,721; 6,294,201; 6,284,276; 6,110,498; 5,573,776; 4,200,0984; and 4,088,864, the contents of which are incorporated herein by reference. One skilled in the art, considering both the disclosure of this invention and the disclosures of these other patents could produce an osmotic pump for the extended release of the polypeptides of the present invention.

Syringe pumps may also be used as slow release agents. Such devices are described in U.S. Pat. Nos. 4,976,696; 4,933,185; 5,017,378; 6,309,370; 6,254,573; 4,435,173; 4,398,908; 6,572,585; 5,298,022; 5,176,502; 5,492,534; 5,318,540; and 4,988,337, the contents of which are incorporated herein by reference. One skilled in the art, considering both the disclosure of this invention and the disclosures of these other patents could produce a syringe pump for the extended release of the compositions of the present invention.

### IX). Pharmaceutical Kits

In another aspect, the invention provides a kit to facilitate the use of the CFXTEN polypeptides. The kit comprises the pharmaceutical composition provided herein, a label identifying the pharmaceutical composition, and an instruction for storage, reconstitution and/or administration of the pharmaceutical compositions to a subject. In some embodiment, the kit comprises, preferably: (a) an amount of a CFXTEN fusion protein composition sufficient to treat a disease, condition or disorder upon administration to a subject in need thereof; and (b) an amount of a pharmaceutically acceptable carrier; together in a formulation ready for injection or for reconstitution with sterile water, buffer, or dextrose; together with a label identifying the CFXTEN drug and storage and handling conditions, and a sheet of the approved indications for the drug, instructions for the reconstitution and/or administration of the CFXTEN drug for the use for the prevention and/or treatment of an approved indication, appropriate dosage and safety information, and information identifying the lot and expiration of the drug. In another embodiment of the foregoing, the kit can comprise a second container that can carry a suitable diluent for the CFXTEN composition, the use of which will provide the user with the appropriate concentration of CFXTEN to be delivered to the subject.

### EXAMPLES

### Example 1

### Construction of XTEN_AD36 Motif Segments

The following example describes the construction of a collection of codon-optimized genes encoding motif sequences of 36 amino acids. As a first step, a stuffer vector pCW0359 was constructed based on a pET vector and that includes a T7 promoter. pCW0359 encodes a cellulose binding domain (CBD) and a TEV protease recognition site followed by a stuffer sequence that is flanked by BsaI, BbsI, and KpnI sites. The BsaI and BbsI sites were inserted such that they generate compatible overhangs after digestion. The stuffer sequence is followed by a truncated version of the GFP gene and a His tag. The stuffer sequence contains stop codons and thus E. coli cells carrying the stuffer plasmid pCW0359 form non-fluorescent colonies. The stuffer vector pCW0359 was digested with BsaI and KpnI to remove the stuffer segment and the resulting vector fragment was isolated by agarose gel purification. The sequences were designated XTEN_AD36, reflecting the AD family of motifs. Its segments have the amino acid sequence [X]₃ where X is a 12 mer peptide with the sequences: GESPGGSSGSES, GSEGSSGPGESS, GSSESGSSEGGP, or GSGGEPSESGSS. The insert was obtained by annealing the following pairs of phosphorylated synthetic oligonucleotide pairs:

| | |
|---|---|
| AD1for: | AGGTGAATCTCCDGGTGGYTCYAGCGGTTCYGARTC |
| AD1rev: | ACCTGAYTCRGAACCGCTRGARCCACCHGGAGATTC |
| AD2for: | AGGTAGCGAAGGTTCTTCYGGTCCDGGYGARTCYTC |
| AD2rev: | ACCTGARGAYTCRCCHGGACCRGAAGAACCTTCGCT |
| AD3for: | AGGTTCYTCYGAAAGCGGTTCTTCYGARGGYGGTCC |
| AD3rev: | ACCTGGACCRCCYTCRGAAGAACCGCTTTCRGARGA |
| AD4for: | AGGTTCYGGTGGYGAACCDTCYGARTCTGGTAGCTC |

We also annealed the phosphorylated oligonucleotide “3KpnIstopperFor”: AGGTTCGCCTTCACTCGAGGGTAC and the non-phosphorylated oligonucleotide pr_{—}3KpnIstopperRev: CCTCGAGTGAAGACGA. The annealed oligonucleotide pairs were ligated, which resulted in a mixture of products with varying length that represents the varying number of 12 mer repeats ligated to one BbsI/KpnI segment. The products corresponding to the length of 36 amino acids were isolated from the mixture by preparative agarose gel electrophoresis and ligated into the BsaI/KpnI digested stuffer vector pCW0359. Most of the clones in the resulting library designated LCW0401 showed green fluorescence after induction, which shows that the sequence of XTEN_AD36 had been ligated in frame with the GFP gene and that most sequences of XTEN_AD36 had good expression levels.

We screened 96 isolates from library LCW0401 for high level of fluorescence by stamping them onto agar plate containing IPTG. The same isolates were evaluated by PCR and 48 isolates were identified that contained segments with 36 amino acids as well as strong fluorescence. These isolates were sequenced and 39 clones were identified that contained correct XTEN_AD36 segments. The file names of the nucleotide and amino acid constructs for these segments are listed in Table 9.

TABLE 9
DNA and Amino Acid Sequences for 36-mer motifs

| File name | Amino acid sequence | Nucleotide sequence |
|---|---|---|
| LCW0401_001_GFP- | GSGGEPSESGSSGESPG | GGTTCTGGTGGCGAACCGTCCGAGTCTGGTAGCTCA |
| N_A01.ab1 | GSSGSESGESPGGSSGS | GGTGAATCTCCGGGTGGCTCTAGCGGTTCCGAGTCA |
| | ES | GGTGAATCTCCTGGTGGTTCCAGCGGTTCCGAGTCA |
| LCW0401_002_GFP- | GSEGSSGPGESSGESPG | GGTAGCGAAGGTTCTTCTGGTCCTGGCGAGTCTTCA |
| N_B01.ab1 | GSSGSESGSSESGSSEG | GGTGAATCTCCTGGTGGTTCCAGCGGTTCTGAATCA |
| | GP | GGTTCCTCCGAAAGCGGTTCTTCCGAGGGCGGTCCA |
| LCW0401_003_GFP- | GSSESGSSEGGPGSSES | GGTTCCTCTGAAAGCGGTTCTTCCGAAGGTGGTCCA |
| N_C01.ab1 | GSSEGGPGESPGGSSG | GGTTCCTCTGAAAGCGGTTCTTCTGAGGGTGGTCCA |
| | SES | GGTGAATCTCCGGGTGGCTCCAGCGGTTCCGAGTCA |
| LCW0401_004_GFP- | GSGGEPSESGSSGSSES | GGTTCCGGTGGCGAACCGTCTGAATCTGGTAGCTCA |
| N_D01.ab1 | GSSEGGPGSGGEPSES | GGTTCTTCTGAAAGCGGTTCTTCCGAGGGTGGTCCA |
| | GSS | GGTTCTGGTGGTGAACCTTCCGAGTCTGGTAGCTCA |
| LCW0401_007_GFP- | GSSESGSSEGGPGSEGS | GGTTCTTCCGAAAGCGGTTCTTCTGAGGGTGGTCCA |
| N_F01.ab1 | SGPGESSGSEGSSGPGE | GGTAGCGAAGGTTCTTCCGGTCCAGGTGAGTCTTCA |
| | SS | GGTAGCGAAGGTTCTTCTGGTCCTGGTGAATCTTCA |
| LCW0401_008_GFP- | GSSESGSSEGGPGESPG | GGTTCCTCTGAAAGCGGTTCTTCCGAGGGTGGTCCA |
| N_G01.ab1 | GSSGSESGSEGSSGPGE | GGTGAATCTCCAGGTGGTTCCAGCGGTTCTGAGTCA |
| | SS | GGTAGCGAAGGTTCTTCTGGTCCAGGTGAATCCTCA |
| LCW0401_012_GFP- | GSGGEPSESGSSGSGG | GGTTCTGGTGGTGAACCGTCTGAGTCTGGTAGCTCA |
| N_H01.ab1 | EPSESGSSGSEGSSGPG | GGTTCCGGTGGCGAACCATCCGAATCTGGTAGCTCA |
| | ESS | GGTAGCGAAGGTTCTTCCGGTCCAGGTGAGTCTTCA |
| LCW0401_015_GFP- | GSSESGSSEGGPGSEGS | GGTTCTTCCGAAAGCGGTTCTTCCGAAGGCGGTCCA |
| N_A02.ab1 | SGPGESSGESPGGSSGS | GGTAGCGAAGGTTCTTCTGGTCCAGGCGAATCTTCA |
| | ES | GGTGAATCTCCTGGTGGCTCCAGCGGTTCTGAGTCA |
| LCW0401_016_GFP- | GSSESGSSEGGPGSSES | GGTTCCTCCGAAAGCGGTTCTTCTGAGGGCGGTCCA |
| N_B02.ab1 | GSSEGGPGSSESGSSEG | GGTTCCTCCGAAAGCGGTTCTTCCGAGGGCGGTCCA |
| | GP | GGTTCTTCTGAAAGCGGTTCTTCCGAGGGCGGTCCA |
| LCW0401_020_GFP- | GSGGEPSESGSSGSEGS | GGTTCCGGTGGCGAACCGTCCGAATCTGGTAGCTCA |
| N_E02.ab1 | SGPGESSGSSESGSSEG | GGTAGCGAAGGTTCTTCTGGTCCAGGCGAATCTTCA |
| | GP | GGTTCCTCTGAAAGCGGTTCTTCTGAGGGCGGTCCA |
| LCW0401_022_GFP- | GSGGEPSESGSSGSSES | GGTTCTGGTGGTGAACCGTCCGAATCTGGTAGCTCA |
| N_F02.ab1 | GSSEGGPGSGGEPSES | GGTTCTTCCGAAAGCGGTTCTTCTGAAGGTGGTCCA |
| | GSS | GGTTCCGGTGGCGAACCTTCTGAATCTGGTAGCTCA |
| LCW0401_024_GFP- | GSGGEPSESGSSGSSES | GGTTCTGGTGGCGAACCGTCCGAATCTGGTAGCTCA |
| N_G02.ab1 | GSSEGGPGESPGGSSG | GGTTCCTCCGAAAGCGGTTCTTCTGAAGGTGGTCCA |
| | SES | GGTGAATCTCCAGGTGGTTCTAGCGGTTCTGAATCA |
| LCW0401_026_GFP- | GSGGEPSESGSSGESPG | GGTTCTGGTGGCGAACCGTCTGAGTCTGGTAGCTCA |
| N_H02.ab1 | GSSGSESGSEGSSGPGE | GGTGAATCTCCTGGTGGCTCCAGCGGTTCTGAATCA |
| | SS | GGTAGCGAAGGTTCTTCTGGTCCTGGTGAATCTTCA |
| LCW0401_027_GFP- | GSGGEPSESGSSGESPG | GGTTCCGGTGGCGAACCTTCCGAATCTGGTAGCTCA |
| N_A03.ab1 | GSSGSESGSGGEPSESG | GGTGAATCTCCGGGTGGTTCTAGCGGTTCTGAGTCA |
| | SS | GGTTCTGGTGGTGAACCTTCCGAGTCTGGTAGCTCA |
| LCW0401_028_GFP- | GSSESGSSEGGPGSSES | GGTTCCTCTGAAAGCGGTTCTTCTGAGGGCGGTCCA |
| N_B03.ab1 | GSSEGGPGSSESGSSEG | GGTTCTTCCGAAAGCGGTTCTTCCGAGGGCGGTCCA |
| | GP | GGTTCTTCCGAAAGCGGTTCTTCTGAAGGCGGTCCA |
| LCW0401_030_GFP- | GESPGGSSGSESGSEGS | GGTGAATCTCCGGGTGGCTCCAGCGGTTCTGAGTCA |
| N_C03.ab1 | SGPGESSGSEGSSGPGE | GGTAGCGAAGGTTCTTCCGGTCCGGGTGAGTCCTCA |
| | SS | GGTAGCGAAGGTTCTTCCGGTCCTGGTGAGTCTTCA |
| LCW0401_031_GFP- | GSGGEPSESGSSGSGG | GGTTCTGGTGGCGAACCTTCCGAATCTGGTAGCTCA |
| N_D03.ab1 | EPSESGSSGSSESGSSE | GGTTCCGGTGGTGAACCTTCTGAATCTGGTAGCTCA |
| | GGP | GGTTCTTCTGAAAGCGGTTCTTCCGAGGGCGGTCCA |
| LCW0401_033_GFP- | GSGGEPSESGSSGSGG | GGTTCCGGTGGTGAACCTTCTGAATCTGGTAGCTCA |
| N_E03.ab1 | EPSESGSSGSGGEPSES | GGTTCCGGTGGCGAACCATCCGAGTCTGGTAGCTCA |
| | GSS | GGTTCCGGTGGTGAACCATCCGAGTCTGGTAGCTCA |
| LCW0401_037_GFP- | GSGGEPSESGSSGSSES | GGTTCCGGTGGCGAACCTTCTGAATCTGGTAGCTCA |
| N_F03.ab1 | GSSEGGPGSEGSSGPG | GGTTCCTCCGAAAGCGGTTCTTCTGAGGGCGGTCCA |
| | ESS | GGTAGCGAAGGTTCTTCTGGTCCGGGCGAGTCTTCA |
| LCW0401_038_GFP- | GSGGEPSESGSSGSEGS | GGTTCCGGTGGTGAACCGTCCGAGTCTGGTAGCTCA |
| N_G03.ab1 | SGPGESSGSGGEPSESG | GGTAGCGAAGGTTCTTCTGGTCCGGGTGAGTCTTCA |
| | SS | GGTTCTGGTGGCGAACCGTCCGAATCTGGTAGCTCA |
| LCW0401_039_GFP- | GSGGEPSESGSSGESPG | GGTTCTGGTGGCGAACCGTCCGAATCTGGTAGCTCA |
| N_H03.ab1 | GSSGSESGSGGEPSESG | GGTGAATCTCCTGGTGGTTCCAGCGGTTCCGAGTCA |
| | SS | GGTTCTGGTGGCGAACCTTCCGAATCTGGTAGCTCA |
| LCW0401_040_GFP- | GSSESGSSEGGPGSGG | GGTTCTTCCGAAAGCGGTTCTTCCGAGGGCGGTCCA |
| N_A04.ab1 | EPSESGSSGSSESGSSE | GGTTCCGGTGGTGAACCATCTGAATCTGGTAGCTCA |
| | GGP | GGTTCTTCTGAAAGCGGTTCTTCTGAAGGTGGTCCA |
| LCW0401_042_GFP- | GSEGSSGPGESSGESPG | GGTAGCGAAGGTTCTTCCGGTCCTGGTGAGTCTTCA |
| N_C04.ab1 | GSSGSESGSEGSSGPGE | GGTGAATCTCCAGGTGGCTCTAGCGGTTCCGAGTCA |
| | SS | GGTAGCGAAGGTTCTTCTGGTCCTGGCGAGTCCTCA |
| LCW0401_046_GFP- | GSSESGSSEGGPGSSES | GGTTCCTCTGAAAGCGGTTCTTCCGAAGGCGGTCCA |
| N_D04.ab1 | GSSEGGPGSSESGSSEG | GGTTCTTCCGAAAGCGGTTCTTCTGAGGGCGGTCCA |
| | GP | GGTTCCTCCGAAAGCGGTTCTTCTGAGGGTGGTCCA |
| LCW0401_047_GFP- | GSGGEPSESGSSGESPG | GGTTCTGGTGGCGAACCTTCCGAGTCTGGTAGCTCA |
| N_E04.ab1 | GSSGSESGESPGGSSGS | GGTGAATCTCCGGGTGGTTCTAGCGGTTCCGAGTCA |
| | ES | GGTGAATCTCCGGGTGGTTCCAGCGGTTCTGAGTCA |
| LCW0401_051_GFP- | GSGGEPSESGSSGSEGS | GGTTCTGGTGGCGAACCATCTGAGTCTGGTAGCTCA |
| N_F04.ab1 | SGPGESSGESPGGSSGS | GGTAGCGAAGGTTCTTCCGGTCCAGGCGAGTCTTCA |
| | ES | GGTGAATCTCCTGGTGGCTCCAGCGGTTCTGAGTCA |
| LCW0401_053_GFP- | GESPGGSSGSESGESPG | GGTGAATCTCCTGGTGGTTCCAGCGGTTCCGAGTCA |
| N_H04.ab1 | GSSGSESGESPGGSSGS | GGTGAATCTCCAGGTGGCTCTAGCGGTTCCGAGTCA |
| | ES | GGTGAATCTCCTGGTGGTTCTAGCGGTTCTGAATCA |
| LCW0401_054_GFP- | GSEGSSGPGESSGSEGS | GGTAGCGAAGGTTCTTCCGGTCCAGGTGAATCTTCA |
| N_A05.ab1 | SGPGESSGSGGEPSESG | GGTAGCGAAGGTTCTTCTGGTCCTGGTGAATCCTCA |
| | SS | GGTTCCGGTGGCGAACCATCTGAATCTGGTAGCTCA |
| LCW0401_059_GFP- | GSGGEPSESGSSGSEGS | GGTTCTGGTGGCGAACCATCCGAATCTGGTAGCTCA |
| N_D05.ab1 | SGPGESSGESPGGSSGS | GGTAGCGAAGGTTCTTCTGGTCCTGGCGAATCTTCA |
| | ES | GGTGAATCTCCAGGTGGCTCTAGCGGTTCCGAATCA |
| LCW0401_060_GFP- | GSGGEPSESGSSGSSES | GGTTCCGGTGGTGAACCGTCCGAATCTGGTAGCTCA |
| N_E05.ab1 | GSSEGGPGSGGEPSES | GGTTCCTCTGAAAGCGGTTCTTCCGAGGGTGGTCCA |
| | GSS | GGTTCCGGTGGTGAACCTTCTGAGTCTGGTAGCTCA |
| LCW0401_061_GFP- | GSSESGSSEGGPGSGG | GGTTCCTCTGAAAGCGGTTCTTCTGAGGGCGGTCCA |
| N_F05.ab1 | EPSESGSSGSEGSSGPG | GGTTCTGGTGGCGAACCATCTGAATCTGGTAGCTCA |
| | ESS | GGTAGCGAAGGTTCTTCCGGTCCGGGTGAATCTTCA |
| LCW0401_063_GFP- | GSGGEPSESGSSGSEGS | GGTTCTGGTGGTGAACCGTCCGAATCTGGTAGCTCA |
| N_H05.ab1 | SGPGESSGSEGSSGPGE | GGTAGCGAAGGTTCTTCTGGTCCTGGCGAGTCTTCA |
| | SS | GGTAGCGAAGGTTCTTCTGGTCCTGGTGAATCTTCA |
| LCW0401_066_GFP- | GSGGEPSESGSSGSSES | GGTTCTGGTGGCGAACCATCCGAGTCTGGTAGCTCA |
| N_B06.ab1 | GSSEGGPGSGGEPSES | GGTTCTTCCGAAAGCGGTTCTTCCGAAGGCGGTCCA |
| | GSS | GGTTCTGGTGGTGAACCGTCCGAATCTGGTAGCTCA |
| LCW0401_067_GFP- | GSGGEPSESGSSGESPG | GGTTCCGGTGGCGAACCTTCCGAATCTGGTAGCTCA |
| N_C06.ab1 | GSSGSESGESPGGSSGS | GGTGAATCTCCGGGTGGTTCTAGCGGTTCCGAATCA |
| | ES | GGTGAATCTCCAGGTGGTTCTAGCGGTTCCGAATCA |
| LCW0401_069_GFP- | GSGGEPSESGSSGSGG | GGTTCCGGTGGTGAACCATCTGAGTCTGGTAGCTCA |
| N_D06.ab1 | EPSESGSSGESPGGSSG | GGTTCCGGTGGCGAACCGTCCGAGTCTGGTAGCTCA |
| | SES | GGTGAATCTCCGGGTGGTTCCAGCGGTTCCGAATCA |
| LCW0401_070_GFP- | GSEGSSGPGESSGSSES | GGTAGCGAAGGTTCTTCTGGTCCGGGCGAATCCTCA |
| N_E06.ab1 | GSSEGGPGSEGSSGPG | GGTTCCTCCGAAAGCGGTTCTTCCGAAGGTGGTCCA |
| | ESS | GGTAGCGAAGGTTCTTCCGGTCCTGGTGAATCTTCA |
| LCW0401_078_GFP- | GSSESGSSEGGPGESPG | GGTTCCTCTGAAAGCGGTTCTTCTGAAGGCGGTCCA |
| N_F06.ab1 | GSSGSESGESPGGSSGS | GGTGAATCTCCGGGTGGCTCCAGCGGTTCTGAATCA |
| | ES | GGTGAATCTCCTGGTGGCTCCAGCGGTTCCGAGTCA |
| LCW0401_079_GFP- | GSEGSSGPGESSGSEGS | GGTAGCGAAGGTTCTTCTGGTCCAGGCGAGTCTTCA |
| N_G06.ab1 | SGPGESSGSGGEPSESG | GGTAGCGAAGGTTCTTCCGGTCCTGGCGAGTCTTCA |
| | SS | GGTTCCGGTGGCGAACCGTCCGAATCTGGTAGCTCA |

### Example 2

### Construction of XTEN_AE36 Segments

A codon library encoding XTEN sequences of 36 amino acid length was constructed. The XTEN sequence was designated XTEN_AE36. Its segments have the amino acid sequence [X]₃ where X is a 12 mer peptide with the sequence: GSPAGSPTSTEE, GSEPATSGSE TP, GTSESA TPESGP, or GTSTEPSEGSAP. The insert was obtained by annealing the following pairs of phosphorylated synthetic oligonucleotide pairs:

| | |
|---|---|
| AE1for: | AGGTAGCCCDGCWGGYTCTCCDACYTCYACYGARGA |
| AE1rev: | ACCTTCYTCRGTRGARGTHGGAGARCCWGCHGGGCT |
| AE2for: | AGGTAGCGAACCKGCWACYTCYGGYTCTGARACYCC |
| AE2rev: | ACCTGGRGTYTCAGARCCRGARGTWGCMGGTTCGCT |
| AE3for: | AGGTACYTCTGAAAGCGCWACYCCKGARTCYGGYCC |
| AE3rev: | ACCTGGRCCRGAYTCMGGRGTWGCGCTTTCAGARGT |
| AE4for: | AGGTACYTCTACYGAACCKTCYGARGGYAGCGCWCC |
| AE4rev: | ACCTGGWGCGCTRCCYTCRGAMGGTTCRGTAGARGT |

We also annealed the phosphorylated oligonucleotide “3KpnIstopperFor”: AGGTTCGTCTTCACTCGAGGGTAC and the non-phosphorylated oligonucleotide “pr_{—}3KpnIstopperRev”: CCTCGAGTGAAGACGA. The annealed oligonucleotide pairs were ligated, which resulted in a mixture of products with varying length that represents the varying number of 12 mer repeats ligated to one BbsI/KpnI segment. The products corresponding to the length of 36 amino acids were isolated from the mixture by preparative agarose gel electrophoresis and ligated into the BsaI/KpnI digested stuffer vector pCW0359. Most of the clones in the resulting library designated LCW0402 showed green fluorescence after induction which shows that the sequence of XTEN_AE36 had been ligated in frame with the GFP gene and most sequences of XTEN_AE36 show good expression.

We screened 96 isolates from library LCW0402 for high level of fluorescence by stamping them onto agar plate containing IPTG. The same isolates were evaluated by PCR and 48 isolates were identified that contained segments with 36 amino acids as well as strong fluorescence. These isolates were sequenced and 37 clones were identified that contained correct XTEN_AE36 segments, The file names of the nucleotide and amino acid constructs for these segments are listed in Table 10.

TABLE 10
DNA and Amino Acid Sequences for 36-mer motifs

| File name | Amino acid sequence | Nucleotide sequence |
|---|---|---|
| LCW0402_002_GFP- | GSPAGSPTSTEEGTSE | GGTAGCCCGGCAGGCTCTCCGACCTCTACTGAGGAAG |
| N_A07.ab1 | SATPESGPGTSTEPSE | GTACTTCTGAAAGCGCAACCCCGGAGTCCGGCCCAGG |
| | GSAP | TACCTCTACCGAACCGTCTGAGGGCAGCGCACCA |
| LCW0402_003_GFP- | GTSTEPSEGSAPGTST | GGTACTTCTACCGAACCGTCCGAAGGCAGCGCTCCAG |
| N_B07.ab1 | EPSEGSAPGTSTEPSE | GTACCTCTACTGAACCTTCCGAGGGCAGCGCTCCAGGT |
| | GSAP | ACCTCTACCGAACCTTCTGAAGGTAGCGCACCA |
| LCW0402_004_GFP- | GTSTEPSEGSAPGTSE | GGTACCTCTACCGAACCGTCTGAAGGTAGCGCACCAG |
| N_C07.ab1 | SATPESGPGTSESATP | GTACCTCTGAAAGCGCAACTCCTGAGTCCGGTCCAGGT |
| | ESGP | ACTTCTGAAAGCGCAACCCCGGAGTCTGGCCCA |
| LCW0402_005_GFP- | GTSTEPSEGSAPGTSE | GGTACTTCTACTGAACCGTCTGAAGGTAGCGCACCAG |
| N_D07.ab1 | SATPESGPGTSESATP | GTACTTCTGAAAGCGCAACCCCGGAATCCGGCCCAGG |
| | ESGP | TACCTCTGAAAGCGCAACCCCGGAGTCCGGCCCA |
| LCW0402_006_GFP- | GSEPATSGSETPGTSE | GGTAGCGAACCGGCAACCTCCGGCTCTGAAACCCCAG |
| N_E07.ab1 | SATPESGPGSPAGSPT | GTACCTCTGAAAGCGCTACTCCTGAATCCGGCCCAGGT |
| | STEE | AGCCCGGCAGGTTCTCCGACTTCCACTGAGGAA |
| LCW0402_008_GFP- | GTSESATPESGPGSEP | GGTACTTCTGAAAGCGCAACCCCTGAATCCGGTCCAG |
| N_F07.ab1 | ATSGSETPGTSTEPSE | GTAGCGAACCGGCTACTTCTGGCTCTGAGACTCCAGGT |
| | GSAP | ACTTCTACCGAACCGTCCGAAGGTAGCGCACCA |
| LCW0402_009_GFP- | GSPAGSPTSTEEGSPA | GGTAGCCCGGCTGGCTCTCCAACCTCCACTGAGGAAG |
| N_G07.ab1 | GSPTSTEEGSEPATSG | GTAGCCCGGCTGGCTCTCCAACCTCCACTGAAGAAGG |
| | SETP | TAGCGAACCGGCTACCTCCGGCTCTGAAACTCCA |
| LCW0402_011_GFP- | GSPAGSPTSTEEGTSE | GGTAGCCCGGCTGGCTCTCCTACCTCTACTGAGGAAGG |
| N_A08.ab1 | SATPESGPGTSTEPSE | TACTTCTGAAAGCGCTACTCCTGAGTCTGGTCCAGGTA |
| | GSAP | CCTCTACTGAACCGTCCGAAGGTAGCGCTCCA |
| LCW0402_012_GFP- | GSPAGSPTSTEEGSPA | GGTAGCCCTGCTGGCTCTCCGACTTCTACTGAGGAAGG |
| N_B08.ab1 | GSPTSTEEGTSTEPSE | TAGCCCGGCTGGTTCTCCGACTTCTACTGAGGAAGGTA |
| | GSAP | CTTCTACCGAACCTTCCGAAGGTAGCGCTCCA |
| LCW0402_013_GFP- | GTSESATPESGPGTST | GGTACTTCTGAAAGCGCTACTCCGGAGTCCGGTCCAG |
| N_C08.ab1 | EPSEGSAPGTSTEPSE | GTACCTCTACCGAACCGTCCGAAGGCAGCGCTCCAGG |
| | GSAP | TACTTCTACTGAACCTTCTGAGGGTAGCGCTCCA |
| LCW0402_014_GFP- | GTSTEPSEGSAPGSPA | GGTACCTCTACCGAACCTTCCGAAGGTAGCGCTCCAG |
| N_D08.ab1 | GSPTSTEEGTSTEPSE | GTAGCCCGGCAGGTTCTCCTACTTCCACTGAGGAAGGT |
| | GSAP | ACTTCTACCGAACCTTCTGAGGGTAGCGCACCA |
| LCW0402_015_GFP- | GSEPATSGSETPGSPA | GGTAGCGAACCGGCTACTTCCGGCTCTGAGACTCCAG |
| N_E08.ab1 | GSPTSTEEGTSESATP | GTAGCCCTGCTGGCTCTCCGACCTCTACCGAAGAAGGT |
| | ESGP | ACCTCTGAAAGCGCTACCCCTGAGTCTGGCCCA |
| LCW0402_016_GFP- | GTSTEPSEGSAPGTSE | GGTACTTCTACCGAACCTTCCGAGGGCAGCGCACCAG |
| N_F08.ab1 | SATPESGPGTSESATP | GTACTTCTGAAAGCGCTACCCCTGAGTCCGGCCCAGGT |
| | ESGP | ACTTCTGAAAGCGCTACTCCTGAATCCGGTCCA |
| LCW0402_020_GFP- | GTSTEPSEGSAPGSEP | GGTACTTCTACTGAACCGTCTGAAGGCAGCGCACCAG |
| N_G08.ab1 | ATSGSETPGSPAGSPT | GTAGCGAACCGGCTACTTCCGGTTCTGAAACCCCAGGT |
| | STEE | AGCCCAGCAGGTTCTCCAACTTCTACTGAAGAA |
| LCW0402_023_GFP- | GSPAGSPTSTEEGTSE | GGTAGCCCTGCTGGCTCTCCAACCTCCACCGAAGAAG |
| N_A09.ab1 | SATPESGPGSEPATSG | GTACCTCTGAAAGCGCAACCCCTGAATCCGGCCCAGG |
| | SETP | TAGCGAACCGGCAACCTCCGGTTCTGAAACCCCA |
| LCW0402_024_GFP- | GTSESATPESGPGSPA | GGTACTTCTGAAAGCGCTACTCCTGAGTCCGGCCCAGG |
| N_B09.ab1 | GSPTSTEEGSPAGSPT | TAGCCCGGCTGGCTCTCCGACTTCCACCGAGGAAGGT |
| | STEE | AGCCCGGCTGGCTCTCCAACTTCTACTGAAGAA |
| LCW0402_025_GFP- | GTSTEPSEGSAPGTSE | GGTACCTCTACTGAACCTTCTGAGGGCAGCGCTCCAGG |
| N_C09.ab1 | SATPESGPGTSTEPSE | TACTTCTGAAAGCGCTACCCCGGAGTCCGGTCCAGGTA |
| | GSAP | CTTCTACTGAACCGTCCGAAGGTAGCGCACCA |
| LCW0402_026_GFP- | GSPAGSPTSTEEGTST | GGTAGCCCGGCAGGCTCTCCGACTTCCACCGAGGAAG |
| N_D09.ab1 | EPSEGSAPGSEPATSG | GTACCTCTACTGAACCTTCTGAGGGTAGCGCTCCAGGT |
| | SETP | AGCGAACCGGCAACCTCTGGCTCTGAAACCCCA |
| LCW0402_027_GFP- | GSPAGSPTSTEEGTST | GGTAGCCCAGCAGGCTCTCCGACTTCCACTGAGGAAG |
| N_E09.ab1 | EPSEGSAPGTSTEPSE | GTACTTCTACTGAACCTTCCGAAGGCAGCGCACCAGGT |
| | GSAP | ACCTCTACTGAACCTTCTGAGGGCAGCGCTCCA |
| LCW0402_032_GFP- | GSEPATSGSETPGTSE | GGTAGCGAACCTGCTACCTCCGGTTCTGAAACCCCAG |
| N_H09.ab1 | SATPESGPGSPAGSPT | GTACCTCTGAAAGCGCAACTCCGGAGTCTGGTCCAGG |
| | STEE | TAGCCCTGCAGGTTCTCCTACCTCCACTGAGGAA |
| LCW0402_034_GFP- | GTSESATPESGPGTST | GGTACCTCTGAAAGCGCTACTCCGGAGTCTGGCCCAG |
| N_A10.ab1 | EPSEGSAPGTSTEPSE | GTACCTCTACTGAACCGTCTGAGGGTAGCGCTCCAGGT |
| | GSAP | ACTTCTACTGAACCGTCCGAAGGTAGCGCACCA |
| LCW0402_036_GFP- | GSPAGSPTSTEEGTST | GGTAGCCCGGCTGGTTCTCCGACTTCCACCGAGGAAG |
| N_C10.ab1 | EPSEGSAPGTSTEPSE | GTACCTCTACTGAACCTTCTGAGGGTAGCGCTCCAGGT |
| | GSAP | ACCTCTACTGAACCTTCCGAAGGCAGCGCTCCA |
| LCW0402_039_GFP- | GTSTEPSEGSAPGTST | GGTACTTCTACCGAACCGTCCGAGGGCAGCGCTCCAG |
| N_E10.ab1 | EPSEGSAPGTSTEPSE | GTACTTCTACTGAACCTTCTGAAGGCAGCGCTCCAGGT |
| | GSAP | ACTTCTACTGAACCTTCCGAAGGTAGCGCACCA |
| LCW0402_040_GFP- | GSEPATSGSETPGTSE | GGTAGCGAACCTGCAACCTCTGGCTCTGAAACCCCAG |
| N_F10.ab1 | SATPESGPGTSTEPSE | GTACCTCTGAAAGCGCTACTCCTGAATCTGGCCCAGGT |
| | GSAP | ACTTCTACTGAACCGTCCGAGGGCAGCGCACCA |
| LCW0402_041_GFP- | GTSTEPSEGSAPGSPA | GGTACTTCTACCGAACCGTCCGAGGGTAGCGCACCAG |
| N_G10.ab1 | GSPTSTEEGTSTEPSE | GTAGCCCAGCAGGTTCTCCTACCTCCACCGAGGAAGG |
| | GSAP | TACTTCTACCGAACCGTCCGAGGGTAGCGCACCA |
| LCW0402_050_GFP- | GSEPATSGSETPGTSE | GGTAGCGAACCGGCAACCTCCGGCTCTGAAACTCCAG |
| N_A11.ab1 | SATPESGPGSEPATSG | GTACTTCTGAAAGCGCTACTCCGGAATCCGGCCCAGGT |
| | SETP | AGCGAACCGGCTACTTCCGGCTCTGAAACCCCA |
| LCW0402_051_GFP- | GSEPATSGSETPGTSE | GGTAGCGAACCGGCAACTTCCGGCTCTGAAACCCCAG |
| N_B11.ab1 | SATPESGPGSEPATSG | GTACTTCTGAAAGCGCTACTCCTGAGTCTGGCCCAGGT |
| | SETP | AGCGAACCTGCTACCTCTGGCTCTGAAACCCCA |
| LCW0402_059_GFP- | GSEPATSGSETPGSEP | GGTAGCGAACCGGCAACCTCTGGCTCTGAAACTCCAG |
| N_E11.ab1 | ATSGSETPGTSTEPSE | GTAGCGAACCTGCAACCTCCGGCTCTGAAACCCCAGG |
| | GSAP | TACTTCTACTGAACCTTCTGAGGGCAGCGCACCA |
| LCW0402_060_GFP- | GTSESATPESGPGSEP | GGTACTTCTGAAAGCGCTACCCCGGAATCTGGCCCAG |
| N_F11.ab1 | ATSGSETPGSEPATSG | GTAGCGAACCGGCTACTTCTGGTTCTGAAACCCCAGGT |
| | SETP | AGCGAACCGGCTACCTCCGGTTCTGAAACTCCA |
| LCW0402_061_GFP- | GTSTEPSEGSAPGTST | GGTACCTCTACTGAACCTTCCGAAGGCAGCGCTCCAG |
| N_G11.ab1 | EPSEGSAPGTSESATP | GTACCTCTACCGAACCGTCCGAGGGCAGCGCACCAGG |
| | ESGP | TACTTCTGAAAGCGCAACCCCTGAATCCGGTCCA |
| LCW0402_065_GFP- | GSEPATSGSETPGTSE | GGTAGCGAACCGGCAACCTCTGGCTCTGAAACCCCAG |
| N_A12.ab1 | SATPESGPGTSESATP | GTACCTCTGAAAGCGCTACTCCGGAATCTGGTCCAGGT |
| | ESGP | ACTTCTGAAAGCGCTACTCCGGAATCCGGTCCA |
| LCW0402_066_GFP- | GSEPATSGSETPGSEP | GGTAGCGAACCTGCTACCTCCGGCTCTGAAACTCCAG |
| N_B12.ab1 | ATSGSETPGTSTEPSE | GTAGCGAACCGGCTACTTCCGGTTCTGAAACTCCAGGT |
| | GSAP | ACCTCTACCGAACCTTCCGAAGGCAGCGCACCA |
| LCW0402_067_GFP- | GSEPATSGSETPGTST | GGTAGCGAACCTGCTACTTCTGGTTCTGAAACTCCAGG |
| N_C12.ab1 | EPSEGSAPGSEPATSG | TACTTCTACCGAACCGTCCGAGGGTAGCGCTCCAGGTA |
| | SETP | GCGAACCTGCTACTTCTGGTTCTGAAACTCCA |
| LCW0402_069_GFP- | GTSTEPSEGSAPGTST | GGTACCTCTACCGAACCGTCCGAGGGTAGCGCACCAG |
| N_D12.ab1 | EPSEGSAPGSEPATSG | GTACCTCTACTGAACCGTCTGAGGGTAGCGCTCCAGGT |
| | SETP | AGCGAACCGGCAACCTCCGGTTCTGAAACTCCA |
| LCW0402_073_GFP- | GTSTEPSEGSAPGSEP | GGTACTTCTACTGAACCTTCCGAAGGTAGCGCTCCAGG |
| N_F12.ab1 | ATSGSETPGSPAGSPT | TAGCGAACCTGCTACTTCTGGTTCTGAAACCCCAGGTA |
| | STEE | GCCCGGCTGGCTCTCCGACCTCCACCGAGGAA |
| LCW0402_074_GFP- | GSEPATSGSETPGSPA | GGTAGCGAACCGGCTACTTCCGGCTCTGAGACTCCAG |
| N_G12.ab1 | GSPTSTEEGTSESATP | GTAGCCCAGCTGGTTCTCCAACCTCTACTGAGGAAGGT |
| | ESGP | ACTTCTGAAAGCGCTACCCCTGAATCTGGTCCA |
| LCW0402_075_GFP- | GTSESATPESGPGSEP | GGTACCTCTGAAAGCGCAACTCCTGAGTCTGGCCCAG |
| N_H12.ab1 | ATSGSETPGTSESATP | GTAGCGAACCTGCTACCTCCGGCTCTGAGACTCCAGGT |
| | ESGP | ACCTCTGAAAGCGCAACCCCGGAATCTGGTCCA |

### Example 3

### Construction of XTEN_AF36 Segments

A codon library encoding sequences of 36 amino acid length was constructed. The sequences were designated XTEN_AF36. Its segments have the amino acid sequence [X]₃ where X is a 12 mer peptide with the sequence: GSTSESPSGTAP, GTSTPESGSASP, GTSPSGESSTAP, or GSTSSTAESPGP. The insert was obtained by annealing the following pairs of phosphorylated synthetic oligonucleotide pairs:

| | |
|---|---|
| AF1for: | AGGTTCTACYAGCGAATCYCCKTCTGGYACYGCWCC |
| AF1rev: | ACCTGGWGCRGTRCCAGAMGGRGATTCGCTRGTAGA |
| AF2for: | AGGTACYTCTACYCCKGAAAGCGGYTCYGCWTCTCC |
| AF2rev: | ACCTGGAGAWGCRGARCCGCTTTCMGGRGTAGARGT |
| AF3for: | AGGTACYTCYCCKAGCGGYGAATCTTCTACYGCWCC |
| AF3rev: | ACCTGGWGCRGTAGAAGATTCRCCGCTMGGRGARGT |
| AF4for: | AGGTTCYACYAGCTCTACYGCWGAATCTCCKGGYCC |
| AF4rev: | ACCTGGRCCMGGAGATTCWGCRGTAGAGCTRGTRGA |

We also annealed the phosphorylated oligonucleotide “3KpnIstopperFor”: AGGTTCGTCTTCACTCGAGGGTAC and the non-phosphorylated oligonucleotide “pr_{—}3KpnIstopperRev”: CCTCGAGTGAAGACGA. The annealed oligonucleotide pairs were ligated, which resulted in a mixture of products with varying length that represents the varying number of 12 mer repeats ligated to one BbsI/KpnI segment The products corresponding to the length of 36 amino acids were isolated from the mixture by preparative agarose gel electrophoresis and ligated into the BsaI/KpnI digested stuffer vector pCW0359. Most of the clones in the resulting library designated LCW0403 showed green fluorescence after induction which shows that the sequence of XTEN_AF36 had been ligated in frame with the GFP gene and most sequences of XTEN_AF36 show good expression.

We screened 96 isolates from library LCW0403 for high level of fluorescence by stamping them onto agar plate containing IPTG. The same isolates were evaluated by PCR and 48 isolates were identified that contained segments with 36 amino acids as well as strong fluorescence. These isolates were sequenced and 44 clones were identified that contained correct XTEN_AF36 segments. The file names of the nucleotide and amino acid constructs for these segments are listed in Table 11.

TABLE 11
DNA and Amino Acid Sequences for 36-mer motifs

| File name | Amino acid sequence | Nucleotide sequence |
|---|---|---|
| LCW0403_004_GFP- | GTSTPESGSASPGTSP | GGTACTTCTACTCCGGAAAGCGGTTCCGCATCTCCA |
| N_A01.ab1 | SGESSTAPGTSPSGES | GGTACTTCTCCTAGCGGTGAATCTTCTACTGCTCCAG |
| | STAP | GTACCTCTCCTAGCGGCGAATCTTCTACTGCTCCA |
| LCW0403_005_GFP- | GTSPSGESSTAPGSTS | GGTACTTCTCCGAGCGGTGAATCTTCTACCGCACCA |
| N_B01.ab1 | STAESPGPGTSPSGES | GGTTCTACTAGCTCTACCGCTGAATCTCCGGGCCCAG |
| | STAP | GTACTTCTCCGAGCGGTGAATCTTCTACTGCTCCA |
| LCW0403_006_GFP- | GSTSSTAESPGPGTSP | GGTTCCACCAGCTCTACTGCTGAATCTCCTGGTCCAG |
| N_C01.ab1 | SGESSTAPGTSTPESG | GTACCTCTCCTAGCGGTGAATCTTCTACTGCTCCAGG |
| | SASP | TACTTCTACTCCTGAAAGCGGCTCTGCTTCTCCA |
| LCW0403_007_GFP- | GSTSSTAESPGPGSTS | GGTTCTACCAGCTCTACTGCAGAATCTCCTGGCCCAG |
| N_D01.ab1 | STAESPGPGTSPSGES | GTTCCACCAGCTCTACCGCAGAATCTCCGGGTCCAG |
| | STAP | GTACTTCCCCTAGCGGTGAATCTTCTACCGCACCA |
| LCW0403_008_GFP- | GSTSSTAESPGPGTSP | GGTTCTACTAGCTCTACTGCTGAATCTCCTGGCCCAG |
| N_E01.ab1 | SGESSTAPGTSTPESG | GTACTTCTCCTAGCGGTGAATCTTCTACCGCTCCAGG |
| | SASP | TACCTCTACTCCGGAAAGCGGTTCTGCATCTCCA |
| LCW0403_010_GFP- | GSTSSTAESPGPGTST | GGTTCTACCAGCTCTACCGCAGAATCTCCTGGTCCAG |
| N_F01.ab1 | PESGSASPGSTSESPS | GTACCTCTACTCCGGAAAGCGGCTCTGCATCTCCAG |
| | GTAP | GTTCTACTAGCGAATCTCCTTCTGGCACTGCACCA |
| LCW0403_011_GFP- | GSTSSTAESPGPGTST | GGTTCTACTAGCTCTACTGCAGAATCTCCTGGCCCAG |
| N_G01.ab1 | PESGSASPGTSTPESG | GTACCTCTACTCCGGAAAGCGGCTCTGCATCTCCAG |
| | SASP | GTACTTCTACCCCTGAAAGCGGTTCTGCATCTCCA |
| LCW0403_012_GFP- | GSTSESPSGTAPGTSP | GGTTCTACCAGCGAATCTCCTTCTGGCACCGCTCCAG |
| N_H01.ab1 | SGESSTAPGSTSESPS | GTACCTCTCCTAGCGGCGAATCTTCTACCGCTCCAGG |
| | GTAP | TTCTACTAGCGAATCTCCTTCTGGCACTGCACCA |
| LCW0403_013_GFP- | GSTSSTAESPGPGSTS | GGTTCCACCAGCTCTACTGCAGAATCTCCGGGCCCA |
| N_A02.ab1 | STAESPGPGTSPSGES | GGTTCTACTAGCTCTACTGCAGAATCTCCGGGTCCAG |
| | STAP | GTACTTCTCCTAGCGGCGAATCTTCTACCGCTCCA |
| LCW0403_014_GFP- | GSTSSTAESPGPGTST | GGTTCCACTAGCTCTACTGCAGAATCTCCTGGCCCAG |
| N_B02.ab1 | PESGSASPGSTSESPS | GTACCTCTACCCCTGAAAGCGGCTCTGCATCTCCAG |
| | GTAP | GTTCTACCAGCGAATCCCCGTCTGGCACCGCACCA |
| LCW0403_015_GFP- | GSTSSTAESPGPGSTS | GGTTCTACTAGCTCTACTGCTGAATCTCCGGGTCCAG |
| N_C02.ab1 | STAESPGPGTSPSGES | GTTCTACCAGCTCTACTGCTGAATCTCCTGGTCCAGG |
| | STAP | TACCTCCCCGAGCGGTGAATCTTCTACTGCACCA |
| LCW0403_017_GFP- | GSTSSTAESPGPGSTS | GGTTCTACCAGCTCTACCGCTGAATCTCCTGGCCCAG |
| N_D02.ab1 | ESPSGTAPGSTSSTAE | GTTCTACCAGCGAATCCCCGTCTGGCACCGCACCAG |
| | SPGP | GTTCTACTAGCTCTACCGCTGAATCTCCGGGTCCA |
| LCW0403_018_GFP- | GSTSSTAESPGPGSTS | GGTTCTACCAGCTCTACCGCAGAATCTCCTGGCCCA |
| N_E02.ab1 | STAESPGPGSTSSTAE | GGTTCCACTAGCTCTACCGCTGAATCTCCTGGTCCAG |
| | SPGP | GTTCTACTAGCTCTACCGCTGAATCTCCTGGTCCA |
| LCW0403_019_GFP- | GSTSESPSGTAPGSTS | GGTTCTACTAGCGAATCCCCTTCTGGTACTGCTCCAG |
| N_F02.ab1 | STAESPGPGSTSSTAE | GTTCCACTAGCTCTACCGCTGAATCTCCTGGCCCAGG |
| | SPGP | TTCCACTAGCTCTACTGCAGAATCTCCTGGTCCA |
| LCW0403_023_GFP- | GSTSESPSGTAPGSTS | GGTTCTACTAGCGAATCTCCTTCTGGTACCGCTCCAG |
| N_H02.ab1 | ESPSGTAPGSTSESPS | GTTCTACCAGCGAATCCCCGTCTGGTACTGCTCCAGG |
| | GTAP | TTCTACCAGCGAATCTCCTTCTGGTACTGCACCA |
| LCW0403_024_GFP- | GSTSSTAESPGPGSTS | GGTTCCACCAGCTCTACTGCTGAATCTCCTGGCCCAG |
| N_A03.ab1 | STAESPGPGSTSSTAE | GTTCTACCAGCTCTACTGCTGAATCTCCGGGCCCAGG |
| | SPGP | TTCCACCAGCTCTACCGCTGAATCTCCGGGTCCA |
| LCW0403_025_GFP- | GSTSSTAESPGPGSTS | GGTTCCACTAGCTCTACCGCAGAATCTCCTGGTCCAG |
| N_B03.ab1 | STAESPGPGTSPSGES | GTTCTACTAGCTCTACTGCTGAATCTCCGGGTCCAGG |
| | STAP | TACCTCCCCTAGCGGCGAATCTTCTACCGCTCCA |
| LCW0403_028_GFP- | GSSPSASTGTGPGSST | GGTTCTAGCCCTTCTGCTTCCACCGGTACCGGCCCAG |
| N_D03.ab1 | PSGATGSPGSSTPSGA | GTAGCTCTACTCCGTCTGGTGCAACTGGCTCTCCAGG |
| | TGSP | TAGCTCTACTCCGTCTGGTGCAACCGGCTCCCCA |
| LCW0403_029_GFP- | GTSPSGESSTAPGTST | GGTACTTCCCCTAGCGGTGAATCTTCTACTGCTCCAG |
| N_E03.ab1 | PESGSASPGSTSSTAE | GTACCTCTACTCCGGAAAGCGGCTCCGCATCTCCAG |
| | SPGP | GTTCTACTAGCTCTACTGCTGAATCTCCTGGTCCA |
| LCW0403_030_GFP- | GSTSSTAESPGPGSTS | GGTTCTACTAGCTCTACCGCTGAATCTCCGGGTCCAG |
| N_F03.ab1 | STAESPGPGTSTPESG | GTTCTACCAGCTCTACTGCAGAATCTCCTGGCCCAGG |
| | SASP | TACTTCTACTCCGGAAAGCGGTTCCGCTTCTCCA |
| LCW0403_031_GFP- | GTSPSGESSTAPGSTS | GGTACTTCTCCTAGCGGTGAATCTTCTACCGCTCCAG |
| N_G03.ab1 | STAESPGPGTSTPESG | GTTCTACCAGCTCTACTGCTGAATCTCCTGGCCCAGG |
| | SASP | TACTTCTACCCCGGAAAGCGGCTCCGCTTCTCCA |
| LCW0403_033_GFP- | GSTSESPSGTAPGSTS | GGTTCTACTAGCGAATCCCCTTCTGGTACTGCACCAG |
| N_H03.ab1 | STAESPGPGSTSSTAE | GTTCTACCAGCTCTACTGCTGAATCTCCGGGCCCAGG |
| | SPGP | TTCCACCAGCTCTACCGCAGAATCTCCTGGTCCA |
| LCW0403_035_GFP- | GSTSSTAESPGPGSTS | GGTTCCACCAGCTCTACCGCTGAATCTCCGGGCCCA |
| N_A04.ab1 | ESPSGTAPGSTSSTAE | GGTTCTACCAGCGAATCCCCTTCTGGCACTGCACCA |
| | SPGP | GGTTCTACTAGCTCTACCGCAGAATCTCCGGGCCCA |
| LCW0403_036_GFP- | GSTSSTAESPGPGTSP | GGTTCTACCAGCTCTACTGCTGAATCTCCGGGTCCAG |
| N_B04.ab1 | SGESSTAPGTSTPESG | GTACTTCCCCGAGCGGTGAATCTTCTACTGCACCAG |
| | SASP | GTACTTCTACTCCGGAAAGCGGTTCCGCTTCTCCA |
| LCW0403_039_GFP- | GSTSESPSGTAPGSTS | GGTTCTACCAGCGAATCTCCTTCTGGCACCGCTCCAG |
| N_C04.ab1 | ESPSGTAPGTSPSGES | GTTCTACTAGCGAATCCCCGTCTGGTACCGCACCAG |
| | STAP | GTACTTCTCCTAGCGGCGAATCTTCTACCGCACCA |
| LCW0403_041_GFP- | GSTSESPSGTAPGSTS | GGTTCTACCAGCGAATCCCCTTCTGGTACTGCTCCAG |
| N_D04.ab1 | ESPSGTAPGTSTPESG | GTTCTACCAGCGAATCCCCTTCTGGCACCGCACCAG |
| | SASP | GTACTTCTACCCCTGAAAGCGGCTCCGCTTCTCCA |
| LCW0403_044_GFP- | GTSTPESGSASPGSTS | GGTACCTCTACTCCTGAAAGCGGTTCTGCATCTCCAG |
| N_E04.ab1 | STAESPGPGSTSSTAE | GTTCCACTAGCTCTACCGCAGAATCTCCGGGCCCAG |
| | SPGP | GTTCTACTAGCTCTACTGCTGAATCTCCTGGCCCA |
| LCW0403_046_GFP- | GSTSESPSGTAPGSTS | GGTTCTACCAGCGAATCCCCTTCTGGCACTGCACCA |
| N_F04.ab1 | ESPSGTAPGTSPSGES | GGTTCTACTAGCGAATCCCCTTCTGGTACCGCACCAG |
| | STAP | GTACTTCTCCGAGCGGCGAATCTTCTACTGCTCCA |
| LCW0403_047_GFP- | GSTSSTAESPGPGSTS | GGTTCTACTAGCTCTACCGCTGAATCTCCTGGCCCAG |
| N_G04.ab1 | STAESPGPGSTSESPS | GTTCCACTAGCTCTACCGCAGAATCTCCGGGCCCAG |
| | GTAP | GTTCTACTAGCGAATCCCCTTCTGGTACCGCTCCA |
| LCW0403_049_GFP- | GSTSSTAESPGPGSTS | GGTTCCACCAGCTCTACTGCAGAATCTCCTGGCCCA |
| N_H04.ab1 | STAESPGPGTSTPESG | GGTTCTACTAGCTCTACCGCAGAATCTCCTGGTCCAG |
| | SASP | GTACCTCTACTCCTGAAAGCGGTTCCGCATCTCCA |
| LCW0403_051_GFP- | GSTSSTAESPGPGSTS | GGTTCTACTAGCTCTACTGCTGAATCTCCGGGCCCAG |
| N_A05.ab1 | STAESPGPGSTSESPS | GTTCTACTAGCTCTACCGCTGAATCTCCGGGTCCAGG |
| | GTAP | TTCTACTAGCGAATCTCCTTCTGGTACCGCTCCA |
| LCW0403_053_GFP- | GTSPSGESSTAPGSTS | GGTACCTCCCCGAGCGGTGAATCTTCTACTGCACCA |
| N_B05.ab1 | ESPSGTAPGSTSSTAE | GGTTCTACTAGCGAATCCCCTTCTGGTACTGCTCCAG |
| | SPGP | GTTCCACCAGCTCTACTGCAGAATCTCCGGGTCCA |
| LCW0403_054_GFP- | GSTSESPSGTAPGTSP | GGTTCTACTAGCGAATCCCCGTCTGGTACTGCTCCAG |
| N_C05.ab1 | SGESSTAPGSTSSTAE | GTACTTCCCCTAGCGGTGAATCTTCTACTGCTCCAGG |
| | SPGP | TTCTACCAGCTCTACCGCAGAATCTCCGGGTCCA |
| LCW0403_057_GFP- | GSTSSTAESPGPGSTS | GGTTCTACCAGCTCTACCGCTGAATCTCCTGGCCCAG |
| N_D05.ab1 | ESPSGTAPGTSPSGES | GTTCTACTAGCGAATCTCCGTCTGGCACCGCACCAG |
| | STAP | GTACTTCCCCTAGCGGTGAATCTTCTACTGCACCA |
| LCW0403_058_GFP- | GSTSESPSGTAPGSTS | GGTTCTACTAGCGAATCTCCTTCTGGCACTGCACCAG |
| N_E05.ab1 | ESPSGTAPGTSTPESG | GTTCTACCAGCGAATCTCCGTCTGGCACTGCACCAG |
| | SASP | GTACCTCTACCCCTGAAAGCGGTTCCGCTTCTCCA |
| LCW0403_060_GFP- | GTSTPESGSASPGSTS | GGTACCTCTACTCCGGAAAGCGGTTCCGCATCTCCA |
| N_F05.ab1 | ESPSGTAPGSTSSTAE | GGTTCTACCAGCGAATCCCCGTCTGGCACCGCACCA |
| | SPGP | GGTTCTACTAGCTCTACTGCTGAATCTCCGGGCCCA |
| LCW0403_063_GFP- | GSTSSTAESPGPGTSP | GGTTCTACTAGCTCTACTGCAGAATCTCCGGGCCCA |
| N_G05.ab1 | SGESSTAPGTSPSGES | GGTACCTCTCCTAGCGGTGAATCTTCTACCGCTCCAG |
| | STAP | GTACTTCTCCGAGCGGTGAATCTTCTACCGCTCCA |
| LCW0403_064_GFP- | GTSPSGESSTAPGTSP | GGTACCTCCCCTAGCGGCGAATCTTCTACTGCTCCAG |
| N_H05.ab1 | SGESSTAPGTSPSGES | GTACCTCTCCTAGCGGCGAATCTTCTACCGCTCCAGG |
| | STAP | TACCTCCCCTAGCGGTGAATCTTCTACCGCACCA |
| LCW0403_065_GFP- | GSTSSTAESPGPGTST | GGTTCCACTAGCTCTACTGCTGAATCTCCTGGCCCAG |
| N_A06.ab1 | PESGSASPGSTSESPS | GTACTTCTACTCCGGAAAGCGGTTCCGCTTCTCCAGG |
| | GTAP | TTCTACTAGCGAATCTCCGTCTGGCACCGCACCA |
| LCW0403_066_GFP- | GSTSESPSGTAPGTSP | GGTTCTACTAGCGAATCTCCGTCTGGCACTGCTCCAG |
| N_B06.ab1 | SGESSTAPGTSPSGES | GTACTTCTCCTAGCGGTGAATCTTCTACCGCTCCAGG |
| | STAP | TACTTCCCCTAGCGGCGAATCTTCTACCGCTCCA |
| LCW0403_067_GFP- | GSTSESPSGTAPGTST | GGTTCTACTAGCGAATCTCCTTCTGGTACCGCTCCAG |
| N_C06.ab1 | PESGSASPGSTSSTAE | GTACTTCTACCCCTGAAAGCGGCTCCGCTTCTCCAGG |
| | SPGP | TTCCACTAGCTCTACCGCTGAATCTCCGGGTCCA |
| LCW0403_068_GFP- | GSTSSTAESPGPGSTS | GGTTCCACTAGCTCTACTGCTGAATCTCCTGGCCCAG |
| N_D06.ab1 | STAESPGPGSTSESPS | GTTCTACCAGCTCTACCGCTGAATCTCCTGGCCCAGG |
| | GTAP | TTCTACCAGCGAATCTCCGTCTGGCACCGCACCA |
| LCW0403_069_GFP- | GSTSESPSGTAPGTST | GGTTCTACTAGCGAATCCCCGTCTGGTACCGCACCA |
| N_E06.ab1 | PESGSASPGTSTPESG | GGTACTTCTACCCCGGAAAGCGGCTCTGCTTCTCCAG |
| | SASP | GTACTTCTACCCCGGAAAGCGGCTCCGCATCTCCA |
| LCW0403_070_GFP- | GSTSESPSGTAPGTST | GGTTCTACTAGCGAATCCCCGTCTGGTACTGCTCCAG |
| N_F06.ab1 | PESGSASPGTSTPESG | GTACTTCTACTCCTGAAAGCGGTTCCGCTTCTCCAGG |
| | SASP | TACCTCTACTCCGGAAAGCGGTTCTGCATCTCCA |

### Example 4

### Construction of XTEN_AG36 Segments

A codon library encoding sequences of 36 amino acid length was constructed. The sequences were designated XTEN_AG36. Its segments have the amino acid sequence [α]; where X is a 12 mer peptide with the sequence: GTPGSGTASSSP, GSSTPSGATGSP, GSSPSASTGTGP, or GASPGTSSTGSP. The insert was obtained by annealing the following pairs of phosphorylated synthetic oligonucleotide pairs:

| | |
|---|---|
| AG1for: | AGGTACYCCKGGYAGCGGTACYGCWTCTTCYTCTCC |
| AG1rev: | ACCTGGAGARGAAGAWGCRGTACCGCTRCCMGGRGT |
| AG2for: | AGGTAGCTCTACYCCKTCTGGTGCWACYGGYTCYCC |
| AG2rev: | ACCTGGRGARCCRGTWGCACCAGAMGGRGTAGAGCT |
| AG3for: | AGGTTCTAGCCCKTCTGCWTCYACYGGTACYGGYCC |
| AG3rev: | ACCTGGRCCRGTACCRGTRGAWGCAGAMGGGCTAGA |
| AG4for: | AGGTGCWTCYCCKGGYACYAGCTCTACYGGTTCTCC |
| AG4rev: | ACCTGGAGAACCRGTAGAGCTRGTRCCMGGRGAWGC |

We also annealed the phosphorylated oligonucleotide “3KpnIstopperFor”: AGGTTCGTCTTCACTCGAGGGTAC and the non-phosphorylated oligonucleotide “pr_{—}3KpnIstopperRev”: CCTCGAGTGAAGACGA. The annealed oligonucleotide pairs were ligated, which resulted in a mixture of products with varying length that represents the varying number of 12 mer repeats ligated to one BbsI/KpnI segment. The products corresponding to the length of 36 amino acids were isolated from the mixture by preparative agarose gel electrophoresis and ligated into the BsaI/KpnI digested stuffer vector pCW0359. Most of the clones in the resulting library designated LCW0404 showed green fluorescence after induction which shows that the sequence of XTEN_AG36 had been ligated in frame with the GFP gene and most sequences of XTEN_AG36 show good expression.

We screened 96 isolates from library LCW0404 for high level of fluorescence by stamping them onto agar plate containing IPTG. The same isolates were evaluated by PCR and 48 isolates were identified that contained segments with 36 amino acids as well as strong fluorescence. These isolates were sequenced and 44 clones were identified that contained correct XTEN_AG36 segments. The file names of the nucleotide and amino acid constructs for these segments are listed in Table 12.

TABLE 12
DNA and Amino Acid Sequences for 36-mer motifs

| File name | Amino acid sequence | Nucleotide sequence |
|---|---|---|
| LCW0404_001_GFP- | GASPGTSSTGSPGTPG | GGTGCATCCCCGGGCACTAGCTCTACCGGTTCTCCA |
| N_A07.ab1 | SGTASSSPGSSTPSGA | GGTACTCCTGGTAGCGGTACTGCTTCTTCTTCTCCAG |
| | TGSP | GTAGCTCTACTCCTTCTGGTGCTACTGGTTCTCCA |
| LCW0404_003_GFP- | GSSTPSGATGSPGSSP | GGTAGCTCTACCCCTTCTGGTGCTACCGGCTCTCCAG |
| N_B07.ab1 | SASTGTGPGSSTPSGA | GTTCTAGCCCGTCTGCTTCTACCGGTACCGGTCCAGG |
| | TGSP | TAGCTCTACCCCTTCTGGTGCTACTGGTTCTCCA |
| LCW0404_006_GFP- | GASPGTSSTGSPGSSP | GGTGCATCTCCGGGTACTAGCTCTACCGGTTCTCCAG |
| N_C07.ab1 | SASTGTGPGSSTPSGA | GTTCTAGCCCTTCTGCTTCCACTGGTACCGGCCCAGG |
| | TGSP | TAGCTCTACCCCGTCTGGTGCTACTGGTTCCCCA |
| LCW0404_007_GFP- | GTPGSGTASSSPGSST | GGTACTCCGGGCAGCGGTACTGCTTCTTCCTCTCCAG |
| N_D07.ab1 | PSGATGSPGASPGTSS | GTAGCTCTACCCCTTCTGGTGCAACTGGTTCCCCAGG |
| | TGSP | TGCATCCCCTGGTACTAGCTCTACCGGTTCTCCA |
| LCW0404_009_GFP- | GTPGSGTASSSPGASP | GGTACCCCTGGCAGCGGTACTGCTTCTTCTTCTCCAG |
| N_E07.ab1 | GTSSTGSPGSRPSAST | GTGCTTCCCCTGGTACCAGCTCTACCGGTTCTCCAGG |
| | GTGP | TTCTAGACCTTCTGCATCCACCGGTACTGGTCCA |
| LCW0404_011_GFP- | GASPGTSSTGSPGSST | GGTGCATCTCCTGGTACCAGCTCTACCGGTTCTCCAG |
| N_F07.ab1 | PSGATGSPGASPGTSS | GTAGCTCTACTCCTTCTGGTGCTACTGGCTCTCCAGG |
| | TGSP | TGCTTCCCCGGGTACCAGCTCTACCGGTTCTCCA |
| LCW0404_012_GFP- | GTPGSGTASSSPGSST | GGTACCCCGGGCAGCGGTACCGCATCTTCCTCTCCA |
| N_G07.ab1 | PSGATGSPGSSTPSGA | GGTAGCTCTACCCCGTCTGGTGCTACCGGTTCCCCAG |
| | TGSP | GTAGCTCTACCCCGTCTGGTGCAACCGGCTCCCCA |
| LCW0404_014_GFP- | GASPGTSSTGSPGASP | GGTGCATCTCCGGGCACTAGCTCTACTGGTTCTCCAG |
| N_H07.ab1 | GTSSTGSPGASPGTSS | GTGCATCCCCTGGCACTAGCTCTACTGGTTCTCCAGG |
| | TGSP | TGCTTCTCCTGGTACCAGCTCTACTGGTTCTCCA |
| LCW0404_015_GFP- | GSSTPSGATGSPGSSP | GGTAGCTCTACTCCGTCTGGTGCAACCGGCTCCCCA |
| N_A08.ab1 | SASTGTGPGASPGTSS | GGTTCTAGCCCGTCTGCTTCCACTGGTACTGGCCCAG |
| | TGSP | GTGCTTCCCCGGGCACCAGCTCTACTGGTTCTCCA |
| LCW0404_016_GFP- | GSSTPSGATGSPGSST | GGTAGCTCTACTCCTTCTGGTGCTACCGGTTCCCCAG |
| N_B08.ab1 | PSGATGSPGTPGSGT | GTAGCTCTACTCCTTCTGGTGCTACTGGTTCCCCAGG |
| | ASSSP | TACTCCGGGCAGCGGTACTGCTTCTTCCTCTCCA |
| LCW0404_017_GFP- | GSSTPSGATGSPGSST | GGTAGCTCTACTCCGTCTGGTGCAACCGGTTCCCCAG |
| N_C08.ab1 | PSGATGSPGASPGTSS | GTAGCTCTACTCCTTCTGGTGCTACTGGCTCCCCAGG |
| | TGSP | TGCATCCCCTGGCACCAGCTCTACCGGTTCTCCA |
| LCW0404_018_GFP- | GTPGSGTASSSPGSSP | GGTACTCCTGGTAGCGGTACCGCATCTTCCTCTCCAG |
| N_D08.ab1 | SASTGTGPGSSTPSGA | GTTCTAGCCCTTCTGCATCTACCGGTACCGGTCCAGG |
| | TGSP | TAGCTCTACTCCTTCTGGTGCTACTGGCTCTCCA |
| LCW0404_023_GFP- | GASPGTSSTGSPGSSP | GGTGCTTCCCCGGGCACTAGCTCTACCGGTTCTCCAG |
| N_F08.ab1 | SASTGTGPGTPGSGT | GTTCTAGCCCTTCTGCATCTACTGGTACTGGCCCAGG |
| | ASSSP | TACTCCGGGCAGCGGTACTGCTTCTTCCTCTCCA |
| LCW0404_025_GFP- | GSSTPSGATGSPGSST | GGTAGCTCTACTCCGTCTGGTGCTACCGGCTCTCCAG |
| N_G08.ab1 | PSGATGSPGASPGTSS | GTAGCTCTACCCCTTCTGGTGCAACCGGCTCCCCAGG |
| | TGSP | TGCTTCTCCGGGTACCAGCTCTACTGGTTCTCCA |
| LCW0404_029_GFP- | GTPGSGTASSSPGSST | GGTACCCCTGGCAGCGGTACCGCTTCTTCCTCTCCAG |
| N_A09.ab1 | PSGATGSPGSSPSAST | GTAGCTCTACCCCGTCTGGTGCTACTGGCTCTCCAGG |
| | GTGP | TTCTAGCCCGTCTGCATCTACCGGTACCGGCCCA |
| LCW0404_030_GFP- | GSSTPSGATGSPGTPG | GGTAGCTCTACTCCTTCTGGTGCAACCGGCTCCCCAG |
| N_B09.ab1 | SGTASSSPGTPGSGTA | GTACCCCGGGCAGCGGTACCGCATCTTCCTCTCCAG |
| | SSSP | GTACTCCGGGTAGCGGTACTGCTTCTTCTTCTCCA |
| LCW0404_031_GFP- | GTPGSGTASSSPGSST | GGTACCCCGGGTAGCGGTACTGCTTCTTCCTCTCCAG |
| N_C09.ab1 | PSGATGSPGASPGTSS | GTAGCTCTACCCCTTCTGGTGCAACCGGCTCTCCAGG |
| | TGSP | TGCTTCTCCGGGCACCAGCTCTACCGGTTCTCCA |
| LCW0404_034_GFP- | GSSTPSGATGSPGSST | GGTAGCTCTACCCCGTCTGGTGCTACCGGCTCTCCAG |
| N_D09.ab1 | PSGATGSPGASPGTSS | GTAGCTCTACCCCGTCTGGTGCAACCGGCTCCCCAG |
| | TGSP | GTGCATCCCCGGGTACTAGCTCTACCGGTTCTCCA |
| LCW0404_035_GFP- | GASPGTSSTGSPGTPG | GGTGCTTCTCCGGGCACCAGCTCTACTGGTTCTCCAG |
| N_E09.ab1 | SGTASSSPGSSTPSGA | GTACCCCGGGCAGCGGTACCGCATCTTCTTCTCCAG |
| | TGSP | GTAGCTCTACTCCTTCTGGTGCAACTGGTTCTCCA |
| LCW0404_036_GFP- | GSSPSASTGTGPGSST | GGTTCTAGCCCGTCTGCTTCCACCGGTACTGGCCCAG |
| N_F09.ab1 | PSGATGSPGTPGSGT | GTAGCTCTACCCCGTCTGGTGCAACTGGTTCCCCAGG |
| | ASSSP | TACCCCTGGTAGCGGTACCGCTTCTTCTTCTCCA |
| LCW0404_037_GFP- | GASPGTSSTGSPGSSP | GGTGCTTCTCCGGGCACCAGCTCTACTGGTTCTCCAG |
| N_G09.ab1 | SASTGTGPGSSTPSGA | GTTCTAGCCCTTCTGCATCCACCGGTACCGGTCCAGG |
| | TGSP | TAGCTCTACCCCTTCTGGTGCAACCGGCTCTCCA |
| LCW0404_040_GFP- | GASPGTSSTGSPGSST | GGTGCATCCCCGGGCACCAGCTCTACCGGTTCTCCA |
| N_H09.ab1 | PSGATGSPGSSTPSGA | GGTAGCTCTACCCCGTCTGGTGCTACCGGCTCTCCAG |
| | TGSP | GTAGCTCTACCCCGTCTGGTGCTACTGGCTCTCCA |
| LCW0404_041_GFP- | GTPGSGTASSSPGSST | GGTACCCCTGGTAGCGGTACTGCTTCTTCCTCTCCAG |
| N_A10.ab1 | PSGATGSPGTPGSGT | GTAGCTCTACTCCGTCTGGTGCTACCGGTTCTCCAGG |
| | ASSSP | TACCCCGGGTAGCGGTACCGCATCTTCTTCTCCA |
| LCW0404_043_GFP- | GSSPSASTGTGPGSST | GGTTCTAGCCCTTCTGCTTCCACCGGTACTGGCCCAG |
| N_C10.ab1 | PSGATGSPGSSTPSGA | GTAGCTCTACCCCTTCTGGTGCTACCGGCTCCCCAGG |
| | TGSP | TAGCTCTACTCCTTCTGGTGCAACTGGCTCTCCA |
| LCW0404_045_GFP- | GASPGTSSTGSPGSSP | GGTGCTTCTCCTGGCACCAGCTCTACTGGTTCTCCAG |
| N_D10.ab1 | SASTGTGPGSSPSAST | GTTCTAGCCCTTCTGCTTCTACCGGTACTGGTCCAGG |
| | GTGP | TTCTAGCCCTTCTGCATCCACTGGTACTGGTCCA |
| LCW0404_047_GFP- | GTPGSGTASSSPGASP | GGTACTCCTGGCAGCGGTACCGCTTCTTCTTCTCCAG |
| N_F10.ab1 | GTSSTGSPGASPGTSS | GTGCTTCTCCTGGTACTAGCTCTACTGGTTCTCCAGG |
| | TGSP | TGCTTCTCCGGGCACTAGCTCTACTGGTTCTCCA |
| LCW0404_048_GFP- | GSSTPSGATGSPGASP | GGTAGCTCTACCCCGTCTGGTGCTACCGGTTCCCCAG |
| N_G10.ab1 | GTSSTGSPGSSTPSGA | GTGCTTCTCCTGGTACTAGCTCTACCGGTTCTCCAGG |
| | TGSP | TAGCTCTACCCCGTCTGGTGCTACTGGCTCTCCA |
| LCW0404_049_GFP- | GSSTPSGATGSPGTPG | GGTAGCTCTACCCCGTCTGGTGCTACTGGTTCTCCAG |
| N_H10.ab1 | SGTASSSPGSSTPSGA | GTACTCCGGGCAGCGGTACTGCTTCTTCCTCTCCAGG |
| | TGSP | TAGCTCTACCCCTTCTGGTGCTACTGGCTCTCCA |
| LCW0404_050_GFP- | GASPGTSSTGSPGSSP | GGTGCATCTCCTGGTACCAGCTCTACTGGTTCTCCAG |
| N_A11.ab1 | SASTGTGPGSSTPSGA | GTTCTAGCCCTTCTGCTTCTACCGGTACCGGTCCAGG |
| | TGSP | TAGCTCTACTCCTTCTGGTGCTACCGGTTCTCCA |
| LCW0404_051_GFP- | GSSTPSGATGSPGSST | GGTAGCTCTACCCCGTCTGGTGCTACTGGCTCTCCAG |
| N_B11.ab1 | PSGATGSPGSSTPSGA | GTAGCTCTACTCCTTCTGGTGCTACTGGTTCCCCAGG |
| | TGSP | TAGCTCTACCCCGTCTGGTGCAACTGGCTCTCCA |
| LCW0404_052_GFP- | GASPGTSSTGSPGTPG | GGTGCATCCCCGGGTACCAGCTCTACCGGTTCTCCA |
| N_C11.ab1 | SGTASSSPGASPGTSS | GGTACTCCTGGCAGCGGTACTGCATCTTCCTCTCCAG |
| | TGSP | GTGCTTCTCCGGGCACCAGCTCTACTGGTTCTCCA |
| LCW0404_053_GFP- | GSSTPSGATGSPGSSP | GGTAGCTCTACTCCTTCTGGTGCAACTGGTTCTCCAG |
| N_D11.ab1 | SASTGTGPGASPGTSS | GTTCTAGCCCGTCTGCATCCACTGGTACCGGTCCAGG |
| | TGSP | TGCTTCCCCTGGCACCAGCTCTACCGGTTCTCCA |
| LCW0404_057_GFP- | GASPGTSSTGSPGSST | GGTGCATCTCCTGGTACTAGCTCTACTGGTTCTCCAG |
| N_E11.ab1 | PSGATGSPGSSPSAST | GTAGCTCTACTCCGTCTGGTGCAACCGGCTCTCCAGG |
| | GTGP | TTCTAGCCCTTCTGCATCTACCGGTACTGGTCCA |
| LCW0404_060_GFP- | GTPGSGTASSSPGSST | GGTACTCCTGGCAGCGGTACCGCATCTTCCTCTCCAG |
| N_F11.ab1 | PSGATGSPGASPGTSS | GTAGCTCTACTCCGTCTGGTGCAACTGGTTCCCCAGG |
| | TGSP | TGCTTCTCCGGGTACCAGCTCTACCGGTTCTCCA |
| LCW0404_062_GFP- | GSSTPSGATGSPGTPG | GGTAGCTCTACCCCGTCTGGTGCAACCGGCTCCCCA |
| N_G11.ab1 | SGTASSSPGSSTPSGA | GGTACTCCTGGTAGCGGTACCGCTTCTTCTTCTCCAG |
| | TGSP | GTAGCTCTACTCCGTCTGGTGCTACCGGCTCCCCA |
| LCW0404_066_GFP- | GSSPSASTGTGPGSSP | GGTTCTAGCCCTTCTGCATCCACCGGTACCGGCCCAG |
| N_H11.ab1 | SASTGTGPGASPGTSS | GTTCTAGCCCGTCTGCTTCTACCGGTACTGGTCCAGG |
| | TGSP | TGCTTCTCCGGGTACTAGCTCTACTGGTTCTCCA |
| LCW0404_067_GFP- | GTPGSGTASSSPGSST | GGTACCCCGGGTAGCGGTACCGCTTCTTCTTCTCCAG |
| N_A12.ab1 | PSGATGSPGSNPSAST | GTAGCTCTACTCCGTCTGGTGCTACCGGCTCTCCAGG |
| | GTGP | TTCTAACCCTTCTGCATCCACCGGTACCGGCCCA |
| LCW0404_068_GFP- | GSSPSASTGTGPGSST | GGTTCTAGCCCTTCTGCATCTACTGGTACTGGCCCAG |
| N_B12.ab1 | PSGATGSPGASPGTSS | GTAGCTCTACTCCTTCTGGTGCTACCGGCTCTCCAGG |
| | TGSP | TGCTTCTCCGGGTACTAGCTCTACCGGTTCTCCA |
| LCW0404_069_GFP- | GSSTPSGATGSPGASP | GGTAGCTCTACCCCTTCTGGTGCAACCGGCTCTCCAG |
| N_C12.ab1 | GTSSTGSPGTPGSGTA | GTGCATCCCCGGGTACCAGCTCTACCGGTTCTCCAG |
| | SSSP | GTACTCCGGGTAGCGGTACCGCTTCTTCCTCTCCA |
| LCW0404_070_GFP- | GSSTPSGATGSPGSST | GGTAGCTCTACTCCGTCTGGTGCAACCGGTTCCCCAG |
| N_D12.ab1 | PSGATGSPGSSTPSGA | GTAGCTCTACCCCTTCTGGTGCAACCGGCTCCCCAGG |
| | TGSP | TAGCTCTACCCCTTCTGGTGCAACTGGCTCTCCA |
| LCW0404_073_GFP- | GASPGTSSTGSPGTPG | GGTGCTTCTCCTGGCACTAGCTCTACCGGTTCTCCAG |
| N_E12.ab1 | SGTASSSPGSSTPSGA | GTACCCCTGGTAGCGGTACCGCATCTTCCTCTCCAGG |
| | TGSP | TAGCTCTACTCCTTCTGGTGCTACTGGTTCCCCA |
| LCW0404_075_GFP- | GSSTPSGATGSPGSSP | GGTAGCTCTACCCCGTCTGGTGCTACTGGCTCCCCAG |
| N_F12.ab1 | SASTGTGPGSSPSAST | GTTCTAGCCCTTCTGCATCCACCGGTACCGGTCCAGG |
| | GTGP | TTCTAGCCCGTCTGCATCTACTGGTACTGGTCCA |
| LCW0404_080_GFP- | GASPGTSSTGSPGSSP | GGTGCTTCCCCGGGCACCAGCTCTACTGGTTCTCCAG |
| N_G12.ab1 | SASTGTGPGSSPSAST | GTTCTAGCCCGTCTGCTTCTACTGGTACTGGTCCAGG |
| | GTGP | TTCTAGCCCTTCTGCTTCCACTGGTACTGGTCCA |
| LCW0404_081_GFP- | GASPGTSSTGSPGSSP | GGTGCTTCCCCGGGTACCAGCTCTACCGGTTCTCCAG |
| N_H12.ab1 | SASTGTGPGTPGSGT | GTTCTAGCCCTTCTGCTTCTACCGGTACCGGTCCAGG |
| | ASSSP | TACCCCTGGCAGCGGTACCGCATCTTCCTCTCCA |

### Example 5

### Construction of XTEN_AE864

XTEN_AE864 was constructed from serial dimerization of XTEN_AE36 to AE72, 144, 288, 576 and 864. A collection of XTEN_AE72 segments was constructed from 37 different segments of XTEN_AE36. Cultures of E. coli harboring all 37 different 36-amino acid segments were mixed and plasmids were isolated. This plasmid pool was digested with BsaI/NcoI to generate the small fragment as the insert. The same plasmid pool was digested with BbsI/NcoI to generate the large fragment as the vector. The insert and vector fragments were ligated resulting in a doubling of the length and the ligation mixture was transformed into BL21Gold(DE3) cells to obtain colonies of XTEN_AE72.

This library of XTEN_AE72 segments was designated LCW0406. All clones from LCW0406 were combined and dimerized again using the same process as described above yielding library LCW0410 of XTEN_AE144. All clones from LCW0410 were combined and dimerized again using the same process as described above yielding library LCW0414 of XTEN_AE288. Two isolates LCW0414.001 and LCW0414.002 were randomly picked from the library and sequenced to verify the identities. All clones from LCW0414 were combined and dimerized again using the same process as described above yielding library LCW0418 of XTEN_AE576. We screened 96 isolates from library LCW0418 for high level of GFP fluorescence. 8 isolates with right sizes of inserts by PCR and strong fluorescence were sequenced and 2 isolates (LCW0418.018 and LCW0418.052) were chosen for future use based on sequencing and expression data.

The specific clone pCWO432 of XTEN_AE864 was constructed by combining LCW0418.018 of XTEN_AE576 and LCW0414.002 of XTEN_AE288 using the same dimerization process as described above.

### Example 6

### Construction of XTEN_AM144

A collection of XTEN_AM144 segments was constructed starting from 37 different segments of XTEN_AE36, 44 segments of XTEN_AF36, and 44 segments of XTEN_AG36.

Cultures of E. coli harboring all 125 different 36-amino acid segments were mixed and plasmids were isolated. This plasmid pool was digested with BsaI/NcoI to generate the small fragment as the insert. The same plasmid pool was digested with BbsI/NcoI to generate the large fragment as the vector. The insert and vector fragments were ligated resulting in a doubling of the length and the ligation mixture was transformed into BL21Gold(DE3) cells to obtain colonies of XTEN_AM72.

This library of XTEN_AM72 segments was designated LCW0461. All clones from LCW0461 were combined and dimerized again using the same process as described above yielding library LCW0462. 1512 Isolates from library LCW0462 were screened for protein expression. Individual colonies were transferred into 96 well plates and cultured overnight as starter cultures. These starter cultures were diluted into fresh autoinduction medium and cultured for 20-30 h. Expression was measured using a fluorescence plate reader with excitation at 395 nm and emission at 510 nm. 192 isolates showed high level expression and were submitted to DNA sequencing. Most clones in library LCW0462 showed good expression and similar physicochemical properties suggesting that most combinations of XTEN_AM36 segments yield useful XTEN sequences. 30 isolates from LCW0462 were chosen as a preferred collection of XTEN_AM144 segments for the construction of multifunctional proteins that contain multiple XTEN segments. The file names of the nucleotide and amino acid constructs for these segments are listed in Table 13.

TABLE 13
DNA and amino acid sequences for AM144 segments

| Clone | DNA Sequence | Protein Sequence |
|---|---|---|
| LCW462_r1 | GGTACCCCGGGCAGCGGTACCGCATCTTCCTCTCCAGGTA | GTPGSGTASSSPGSST |
| | GCTCTACCCCGTCTGGTGCTACCGGTTCCCCAGGTAGCTC | PSGATGSPGSSTPSGA |
| | TACCCCGTCTGGTGCAACCGGCTCCCCAGGTAGCCCGGCT | TGSPGSPAGSPTSTEE |
| | GGCTCTCCTACCTCTACTGAGGAAGGTACTTCTGAAAGCG | GTSESATPESGPGTST |
| | CTACTCCTGAGTCTGGTCCAGGTACCTCTACTGAACCGTC | EPSEGSAPGSSPSAST |
| | CGAAGGTAGCGCTCCAGGTTCTAGCCCTTCTGCATCCACC | GTGPGSSPSASTGTGP |
| | GGTACCGGCCCAGGTTCTAGCCCGTCTGCTTCTACCGGTA | GASPGTSSTGSPGTST |
| | CTGGTCCAGGTGCTTCTCCGGGTACTAGCTCTACTGGTTC | EPSEGSAPGTSTEPSE |
| | TCCAGGTACCTCTACCGAACCGTCCGAGGGTAGCGCACC | GSAPGSEPATSGSETP |
| | AGGTACCTCTACTGAACCGTCTGAGGGTAGCGCTCCAGG | |
| | TAGCGAACCGGCAACCTCCGGTTCTGAAACTCCA | |
| LCW462_r5 | GGTTCTACCAGCGAATCCCCTTCTGGCACTGCACCAGGTT | GSTSESPSGTAPGSTS |
| | CTACTAGCGAATCCCCTTCTGGTACCGCACCAGGTACTTC | ESPSGTAPGTSPSGES |
| | TCCGAGCGGCGAATCTTCTACTGCTCCAGGTACCTCTACT | STAPGTSTEPSEGSAP |
| | GAACCTTCCGAAGGCAGCGCTCCAGGTACCTCTACCGAA | GTSTEPSEGSAPGTSE |
| | CCGTCCGAGGGCAGCGCACCAGGTACTTCTGAAAGCGCA | SATPESGPGASPGTSS |
| | ACCCCTGAATCCGGTCCAGGTGCATCTCCTGGTACCAGCT | TGSPGSSTPSGATGSP |
| | CTACCGGTTCTCCAGGTAGCTCTACTCCTTCTGGTGCTAC | GASPGTSSTGSPGSTS |
| | TGGCTCTCCAGGTGCTTCCCCGGGTACCAGCTCTACCGGT | ESPSGTAPGSTSESPS |
| | TCTCCAGGTTCTACTAGCGAATCTCCTTCTGGCACTGCAC | GTAPGTSTPESGSASP |
| | CAGGTTCTACCAGCGAATCTCCGTCTGGCACTGCACCAGG | |
| | TACCTCTACCCCTGAAAGCGGTTCCGCTTCTCCA | |
| LCW462_r9 | GGTACTTCTACCGAACCTTCCGAGGGCAGCGCACCAGGT | GTSTEPSEGSAPGTSE |
| | ACTTCTGAAAGCGCTACCCCTGAGTCCGGCCCAGGTACTT | SATPESGPGTSESATP |
| | CTGAAAGCGCTACTCCTGAATCCGGTCCAGGTACCTCTAC | ESGPGTSTEPSEGSAP |
| | TGAACCTTCTGAGGGCAGCGCTCCAGGTACTTCTGAAAG | GTSESATPESGPGTST |
| | CGCTACCCCGGAGTCCGGTCCAGGTACTTCTACTGAACCG | EPSEGSAPGTSTEPSE |
| | TCCGAAGGTAGCGCACCAGGTACTTCTACTGAACCTTCCG | GSAPGSEPATSGSETP |
| | AAGGTAGCGCTCCAGGTAGCGAACCTGCTACTTCTGGTTC | GSPAGSPTSTEEGASP |
| | TGAAACCCCAGGTAGCCCGGCTGGCTCTCCGACCTCCACC | GTSSTGSPGSSPSAST |
| | GAGGAAGGTGCTTCTCCTGGCACCAGCTCTACTGGTTCTC | GTGPGSSPSASTGTGP |
| | CAGGTTCTAGCCCTTCTGCTTCTACCGGTACTGGTCCAGG | |
| | TTCTAGCCCTTCTGCATCCACTGGTACTGGTCCA | |
| LCW462_r10 | GGTAGCGAACCGGCAACCTCTGGCTCTGAAACCCCAGGT | GSEPATSGSETPGTSE |
| | ACCTCTGAAAGCGCTACTCCGGAATCTGGTCCAGGTACTT | SATPESGPGTSESATP |
| | CTGAAAGCGCTACTCCGGAATCCGGTCCAGGTTCTACCA | ESGPGSTSESPSGTAP |
| | GCGAATCTCCTTCTGGCACCGCTCCAGGTTCTACTAGCGA | GSTSESPSGTAPGTSP |
| | ATCCCCGTCTGGTACCGCACCAGGTACTTCTCCTAGCGGC | SGESSTAPGASPGTSS |
| | GAATCTTCTACCGCACCAGGTGCATCTCCGGGTACTAGCT | TGSPGSSPSASTGTGP |
| | CTACCGGTTCTCCAGGTTCTAGCCCTTCTGCTTCCACTGGT | GSSTPSGATGSPGSST |
| | ACCGGCCCAGGTAGCTCTACCCCGTCTGGTGCTACTGGTT | PSGATGSPGSSTPSGA |
| | CCCCAGGTAGCTCTACTCCGTCTGGTGCAACCGGTTCCCC | TGSPGASPGTSSTGSP |
| | AGGTAGCTCTACTCCTTCTGGTGCTACTGGCTCCCCAGGT | |
| | GCATCCCCTGGCACCAGCTCTACCGGTTCTCCA | |
| LCW462_r15 | GGTGCTTCTCCGGGCACCAGCTCTACTGGTTCTCCAGGTT | GASPGTSSTGSPGSSP |
| | CTAGCCCTTCTGCATCCACCGGTACCGGTCCAGGTAGCTC | SASTGTGPGSSTPSGA |
| | TACCCCTTCTGGTGCAACCGGCTCTCCAGGTACTTCTGAA | TGSPGTSESATPESGP |
| | AGCGCTACCCCGGAATCTGGCCCAGGTAGCGAACCGGCT | GSEPATSGSETPGSEP |
| | ACTTCTGGTTCTGAAACCCCAGGTAGCGAACCGGCTACCT | ATSGSETPGTSESATP |
| | CCGGTTCTGAAACTCCAGGTACTTCTGAAAGCGCTACTCC | ESGPGTSTEPSEGSAP |
| | GGAGTCCGGTCCAGGTACCTCTACCGAACCGTCCGAAGG | GTSTEPSEGSAPGTST |
| | CAGCGCTCCAGGTACTTCTACTGAACCTTCTGAGGGTAGC | EPSEGSAPGTSTEPSE |
| | GCTCCAGGTACCTCTACCGAACCGTCCGAGGGTAGCGCA | GSAPGSEPATSGSETP |
| | CCAGGTACCTCTACTGAACCGTCTGAGGGTAGCGCTCCA | |
| | GGTAGCGAACCGGCAACCTCCGGTTCTGAAACTCCA | |
| LCW462_r16 | GGTACCTCTACCGAACCTTCCGAAGGTAGCGCTCCAGGT | GTSTEPSEGSAPGSPA |
| | AGCCCGGCAGGTTCTCCTACTTCCACTGAGGAAGGTACTT | GSPTSTEEGTSTEPSE |
| | CTACCGAACCTTCTGAGGGTAGCGCACCAGGTACCTCTG | GSAPGTSESATPESGP |
| | AAAGCGCAACTCCTGAGTCTGGCCCAGGTAGCGAACCTG | GSEPATSGSETPGTSE |
| | CTACCTCCGGCTCTGAGACTCCAGGTACCTCTGAAAGCGC | SATPESGPGSPAGSPT |
| | AACCCCGGAATCTGGTCCAGGTAGCCCGGCTGGCTCTCCT | STEEGTSESATPESGP |
| | ACCTCTACTGAGGAAGGTACTTCTGAAAGCGCTACTCCTG | GTSTEPSEGSAPGSEP |
| | AGTCTGGTCCAGGTACCTCTACTGAACCGTCCGAAGGTA | ATSGSETPGTSTEPSE |
| | GCGCTCCAGGTAGCGAACCTGCTACTTCTGGTTCTGAAAC | GSAPGSEPATSGSETP |
| | TCCAGGTACTTCTACCGAACCGTCCGAGGGTAGCGCTCCA | |
| | GGTAGCGAACCTGCTACTTCTGGTTCTGAAACTCCA | |
| LCW462_r20 | GGTACTTCTACCGAACCGTCCGAAGGCAGCGCTCCAGGT | GTSTEPSEGSAPGTST |
| | ACCTCTACTGAACCTTCCGAGGGCAGCGCTCCAGGTACCT | EPSEGSAPGTSTEPSE |
| | CTACCGAACCTTCTGAAGGTAGCGCACCAGGTACTTCTAC | GSAPGTSTEPSEGSAP |
| | CGAACCGTCCGAAGGCAGCGCTCCAGGTACCTCTACTGA | GTSTEPSEGSAPGTST |
| | ACCTTCCGAGGGCAGCGCTCCAGGTACCTCTACCGAACCT | EPSEGSAPGTSTEPSE |
| | TCTGAAGGTAGCGCACCAGGTACTTCTACCGAACCTTCCG | GSAPGTSESATPESGP |
| | AGGGCAGCGCACCAGGTACTTCTGAAAGCGCTACCCCTG | GTSESATPESGPGTST |
| | AGTCCGGCCCAGGTACTTCTGAAAGCGCTACTCCTGAATC | EPSEGSAPGSEPATSG |
| | CGGTCCAGGTACTTCTACTGAACCTTCCGAAGGTAGCGCT | SETPGSPAGSPTSTEE |
| | CCAGGTAGCGAACCTGCTACTTCTGGTTCTGAAACCCCAG | |
| | GTAGCCCGGCTGGCTCTCCGACCTCCACCGAGGAA | |
| LCW462_r23 | GGTACTTCTACCGAACCGTCCGAGGGCAGCGCTCCAGGT | GTSTEPSEGSAPGTST |
| | ACTTCTACTGAACCTTCTGAAGGCAGCGCTCCAGGTACTT | EPSEGSAPGTSTEPSE |
| | CTACTGAACCTTCCGAAGGTAGCGCACCAGGTTCTACCA | GSAPGSTSESPSGTAP |
| | GCGAATCCCCTTCTGGTACTGCTCCAGGTTCTACCAGCGA | GSTSESPSGTAPGTST |
| | ATCCCCTTCTGGCACCGCACCAGGTACTTCTACCCCTGAA | PESGSASPGSEPATSG |
| | AGCGGCTCCGCTTCTCCAGGTAGCGAACCTGCAACCTCTG | SETPGTSESATPESGP |
| | GCTCTGAAACCCCAGGTACCTCTGAAAGCGCTACTCCTGA | GTSTEPSEGSAPGTST |
| | ATCTGGCCCAGGTACTTCTACTGAACCGTCCGAGGGCAG | EPSEGSAPGTSESATP |
| | CGCACCAGGTACTTCTACTGAACCGTCTGAAGGTAGCGC | ESGPGTSESATPESGP |
| | ACCAGGTACTTCTGAAAGCGCAACCCCGGAATCCGGCCC | |
| | AGGTACCTCTGAAAGCGCAACCCCGGAGTCCGGCCCA | |
| LCW462_r24 | GGTAGCTCTACCCCTTCTGGTGCTACCGGCTCTCCAGGTT | GSSTPSGATGSPGSSP |
| | CTAGCCCGTCTGCTTCTACCGGTACCGGTCCAGGTAGCTC | SASTGTGPGSSTPSGA |
| | TACCCCTTCTGGTGCTACTGGTTCTCCAGGTAGCCCTGCT | TGSPGSPAGSPTSTEE |
| | GGCTCTCCGACTTCTACTGAGGAAGGTAGCCCGGCTGGTT | GSPAGSPTSTEEGTST |
| | CTCCGACTTCTACTGAGGAAGGTACTTCTACCGAACCTTC | EPSEGSAPGASPGTSS |
| | CGAAGGTAGCGCTCCAGGTGCTTCCCCGGGCACTAGCTCT | TGSPGSSPSASTGTGP |
| | ACCGGTTCTCCAGGTTCTAGCCCTTCTGCATCTACTGGTA | GTPGSGTASSSPGSTS |
| | CTGGCCCAGGTACTCCGGGCAGCGGTACTGCTTCTTCCTC | STAESPGPGTSPSGES |
| | TCCAGGTTCTACTAGCTCTACTGCTGAATCTCCTGGCCCA | STAPGTSTPESGSASP |
| | GGTACTTCTCCTAGCGGTGAATCTTCTACCGCTCCAGGTA | |
| | CCTCTACTCCGGAAAGCGGTTCTGCATCTCCA | |
| LCW462_r27 | GGTACCTCTACTGAACCTTCTGAGGGCAGCGCTCCAGGTA | GTSTEPSEGSAPGTSE |
| | CTTCTGAAAGCGCTACCCCGGAGTCCGGTCCAGGTACTTC | SATPESGPGTSTEPSE |
| | TACTGAACCGTCCGAAGGTAGCGCACCAGGTACTTCTACT | GSAPGTSTEPSEGSAP |
| | GAACCGTCTGAAGGTAGCGCACCAGGTACTTCTGAAAGC | GTSESATPESGPGTSE |
| | GCAACCCCGGAATCCGGCCCAGGTACCTCTGAAAGCGCA | SATPESGPGTPGSGTA |
| | ACCCCGGAGTCCGGCCCAGGTACTCCTGGCAGCGGTACC | SSSPGASPGTSSTGSP |
| | GCTTCTTCTTCTCCAGGTGCTTCTCCTGGTACTAGCTCTAC | GASPGTSSTGSPGSPA |
| | TGGTTCTCCAGGTGCTTCTCCGGGCACTAGCTCTACTGGT | GSPTSTEEGSPAGSPT |
| | TCTCCAGGTAGCCCTGCTGGCTCTCCGACTTCTACTGAGG | STEEGTSTEPSEGSAP |
| | AAGGTAGCCCGGCTGGTTCTCCGACTTCTACTGAGGAAG | |
| | GTACTTCTACCGAACCTTCCGAAGGTAGCGCTCCA | |
| LCW462_r28 | GGTAGCCCAGCAGGCTCTCCGACTTCCACTGAGGAAGGT | GSPAGSPTSTEEGTST |
| | ACTTCTACTGAACCTTCCGAAGGCAGCGCACCAGGTACCT | EPSEGSAPGTSTEPSE |
| | CTACTGAACCTTCTGAGGGCAGCGCTCCAGGTACCTCTAC | GSAPGTSTEPSEGSAP |
| | CGAACCGTCTGAAGGTAGCGCACCAGGTACCTCTGAAAG | GTSESATPESGPGTSE |
| | CGCAACTCCTGAGTCCGGTCCAGGTACTTCTGAAAGCGC | SATPESGPGTPGSGTA |
| | AACCCCGGAGTCTGGCCCAGGTACCCCGGGTAGCGGTAC | SSSPGSSTPSGATGSP |
| | TGCTTCTTCCTCTCCAGGTAGCTCTACCCCTTCTGGTGCAA | GASPGTSSTGSPGTST |
| | CCGGCTCTCCAGGTGCTTCTCCGGGCACCAGCTCTACCGG | EPSEGSAPGTSESATP |
| | TTCTCCAGGTACCTCTACTGAACCTTCTGAGGGCAGCGCT | ESGPGTSTEPSEGSAP |
| | CCAGGTACTTCTGAAAGCGCTACCCCGGAGTCCGGTCCA | |
| | GGTACTTCTACTGAACCGTCCGAAGGTAGCGCACCA | |
| LCW462_r38 | GGTAGCGAACCGGCAACCTCCGGCTCTGAAACTCCAGGT | GSEPATSGSETPGTSE |
| | ACTTCTGAAAGCGCTACTCCGGAATCCGGCCCAGGTAGC | SATPESGPGSEPATSG |
| | GAACCGGCTACTTCCGGCTCTGAAACCCCAGGTAGCTCTA | SETPGSSTPSGATGSP |
| | CCCCGTCTGGTGCAACCGGCTCCCCAGGTACTCCTGGTAG | GTPGSGTASSSPGSST |
| | CGGTACCGCTTCTTCTTCTCCAGGTAGCTCTACTCCGTCTG | PSGATGSPGASPGTSS |
| | GTGCTACCGGCTCCCCAGGTGCATCTCCTGGTACCAGCTC | TGSPGSSTPSGATGSP |
| | TACCGGTTCTCCAGGTAGCTCTACTCCTTCTGGTGCTACT | GASPGTSSTGSPGSEP |
| | GGCTCTCCAGGTGCTTCCCCGGGTACCAGCTCTACCGGTT | ATSGSETPGTSTEPSE |
| | CTCCAGGTAGCGAACCTGCTACTTCTGGTTCTGAAACTCC | GSAPGSEPATSGSETP |
| | AGGTACTTCTACCGAACCGTCCGAGGGTAGCGCTCCAGG | |
| | TAGCGAACCTGCTACTTCTGGTTCTGAAACTCCA | |
| LCW462_r39 | GGTACCTCTACTGAACCTTCCGAAGGCAGCGCTCCAGGT | GTSTEPSEGSAPGTST |
| | ACCTCTACCGAACCGTCCGAGGGCAGCGCACCAGGTACT | EPSEGSAPGTSESATP |
| | TCTGAAAGCGCAACCCCTGAATCCGGTCCAGGTAGCCCT | ESGPGSPAGSPTSTEE |
| | GCTGGCTCTCCGACTTCTACTGAGGAAGGTAGCCCGGCTG | GSPAGSPTSTEEGTST |
| | GTTCTCCGACTTCTACTGAGGAAGGTACTTCTACCGAACC | EPSEGSAPGSPAGSPT |
| | TTCCGAAGGTAGCGCTCCAGGTAGCCCGGCTGGTTCTCCG | STEEGTSTEPSEGSAP |
| | ACTTCCACCGAGGAAGGTACCTCTACTGAACCTTCTGAGG | GTSTEPSEGSAPGASP |
| | GTAGCGCTCCAGGTACCTCTACTGAACCTTCCGAAGGCA | GTSSTGSPGSSPSAST |
| | GCGCTCCAGGTGCTTCCCCGGGCACCAGCTCTACTGGTTC | GTGPGSSPSASTGTGP |
| | TCCAGGTTCTAGCCCGTCTGCTTCTACTGGTACTGGTCCA | |
| | GGTTCTAGCCCTTCTGCTTCCACTGGTACTGGTCCA | |
| LCW462_r41 | GGTAGCTCTACCCCGTCTGGTGCTACCGGTTCCCCAGGTG | GSSTPSGATGSPGASP |
| | CTTCTCCTGGTACTAGCTCTACCGGTTCTCCAGGTAGCTC | GTSSTGSPGSSTPSGA |
| | TACCCCGTCTGGTGCTACTGGCTCTCCAGGTAGCCCTGCT | TGSPGSPAGSPTSTEE |
| | GGCTCTCCAACCTCCACCGAAGAAGGTACCTCTGAAAGC | GTSESATPESGPGSEP |
| | GCAACCCCTGAATCCGGCCCAGGTAGCGAACCGGCAACC | ATSGSETPGASPGTSS |
| | TCCGGTTCTGAAACCCCAGGTGCATCTCCTGGTACTAGCT | TGSPGSSTPSGATGSP |
| | CTACTGGTTCTCCAGGTAGCTCTACTCCGTCTGGTGCAAC | GSSPSASTGTGPGSTS |
| | CGGCTCTCCAGGTTCTAGCCCTTCTGCATCTACCGGTACT | ESPSGTAPGSTSESPS |
| | GGTCCAGGTTCTACCAGCGAATCCCCTTCTGGTACTGCTC | GTAPGTSTPESGSASP |
| | CAGGTTCTACCAGCGAATCCCCTTCTGGCACCGCACCAGG | |
| | TACTTCTACCCCTGAAAGCGGCTCCGCTTCTCCA | |
| LCW462_r42 | GGTTCTACCAGCGAATCTCCTTCTGGCACCGCTCCAGGTT | GSTSESPSGTAPGSTS |
| | CTACTAGCGAATCCCCGTCTGGTACCGCACCAGGTACTTC | ESPSGTAPGTSPSGES |
| | TCCTAGCGGCGAATCTTCTACCGCACCAGGTACCTCTGAA | STAPGTSESATPESGP |
| | AGCGCTACTCCGGAGTCTGGCCCAGGTACCTCTACTGAAC | GTSTEPSEGSAPGTST |
| | CGTCTGAGGGTAGCGCTCCAGGTACTTCTACTGAACCGTC | EPSEGSAPGTSTEPSE |
| | CGAAGGTAGCGCACCAGGTACCTCTACTGAACCTTCTGA | GSAPGTSESATPESGP |
| | GGGCAGCGCTCCAGGTACTTCTGAAAGCGCTACCCCGGA | GTSTEPSEGSAPGSST |
| | GTCCGGTCCAGGTACTTCTACTGAACCGTCCGAAGGTAGC | PSGATGSPGASPGTSS |
| | GCACCAGGTAGCTCTACCCCGTCTGGTGCTACCGGTTCCC | TGSPGSSTPSGATGSP |
| | CAGGTGCTTCTCCTGGTACTAGCTCTACCGGTTCTCCAGG | |
| | TAGCTCTACCCCGTCTGGTGCTACTGGCTCTCCA | |
| LCW462_r43 | GGTTCTACTAGCTCTACTGCAGAATCTCCGGGCCCAGGTA | GSTSSTAESPGPGTSP |
| | CCTCTCCTAGCGGTGAATCTTCTACCGCTCCAGGTACTTC | SGESSTAPGTSPSGES |
| | TCCGAGCGGTGAATCTTCTACCGCTCCAGGTTCTACTAGC | STAPGSTSSTAESPGP |
| | TCTACCGCTGAATCTCCGGGTCCAGGTTCTACCAGCTCTA | GSTSSTAESPGPGTST |
| | CTGCAGAATCTCCTGGCCCAGGTACTTCTACTCCGGAAAG | PESGSASPGTSPSGES |
| | CGGTTCCGCTTCTCCAGGTACTTCTCCTAGCGGTGAATCT | STAPGSTSSTAESPGP |
| | TCTACCGCTCCAGGTTCTACCAGCTCTACTGCTGAATCTC | GTSTPESGSASPGSTS |
| | CTGGCCCAGGTACTTCTACCCCGGAAAGCGGCTCCGCTTC | STAESPGPGSTSESPS |
| | TCCAGGTTCTACCAGCTCTACCGCTGAATCTCCTGGCCCA | GTAPGTSPSGESSTAP |
| | GGTTCTACTAGCGAATCTCCGTCTGGCACCGCACCAGGTA | |
| | CTTCCCCTAGCGGTGAATCTTCTACTGCACCA | |
| LCW462_r45 | GGTACCTCTACTCCGGAAAGCGGTTCCGCATCTCCAGGTT | GTSTPESGSASPGSTS |
| | CTACCAGCGAATCCCCGTCTGGCACCGCACCAGGTTCTAC | ESPSGTAPGSTSSTAE |
| | TAGCTCTACTGCTGAATCTCCGGGCCCAGGTACCTCTACT | SPGPGTSTEPSEGSAP |
| | GAACCTTCCGAAGGCAGCGCTCCAGGTACCTCTACCGAA | GTSTEPSEGSAPGTSE |
| | CCGTCCGAGGGCAGCGCACCAGGTACTTCTGAAAGCGCA | SATPESGPGTSESATP |
| | ACCCCTGAATCCGGTCCAGGTACCTCTGAAAGCGCTACTC | ESGPGTSTEPSEGSAP |
| | CGGAGTCTGGCCCAGGTACCTCTACTGAACCGTCTGAGG | GTSTEPSEGSAPGTSE |
| | GTAGCGCTCCAGGTACTTCTACTGAACCGTCCGAAGGTA | SATPESGPGTSTEPSE |
| | GCGCACCAGGTACTTCTGAAAGCGCTACTCCGGAGTCCG | GSAPGTSTEPSEGSAP |
| | GTCCAGGTACCTCTACCGAACCGTCCGAAGGCAGCGCTC | |
| | CAGGTACTTCTACTGAACCTTCTGAGGGTAGCGCTCCC | |
| LCW462_r47 | GGTACCTCTACCGAACCGTCCGAGGGTAGCGCACCAGGT | GTSTEPSEGSAPGTST |
| | ACCTCTACTGAACCGTCTGAGGGTAGCGCTCCAGGTAGC | EPSEGSAPGSEPATSG |
| | GAACCGGCAACCTCCGGTTCTGAAACTCCAGGTACTTCTA | SETPGTSTEPSEGSAP |
| | CTGAACCGTCTGAAGGTAGCGCACCAGGTACTTCTGAAA | GTSESATPESGPGTSE |
| | GCGCAACCCCGGAATCCGGCCCAGGTACCTCTGAAAGCG | SATPESGPGASPGTSS |
| | CAACCCCGGAGTCCGGCCCAGGTGCATCTCCGGGTACTA | TGSPGSSPSASTGTGP |
| | GCTCTACCGGTTCTCCAGGTTCTAGCCCTTCTGCTTCCACT | GSSTPSGATGSPGSST |
| | GGTACCGGCCCAGGTAGCTCTACCCCGTCTGGTGCTACTG | PSGATGSPGSSTPSGA |
| | GTTCCCCAGGTAGCTCTACTCCGTCTGGTGCAACCGGTTC | TGSPGASPGTSSTGSP |
| | CCCAGGTAGCTCTACTCCTTCTGGTGCTACTGGCTCCCCA | |
| | GGTGCATCCCCTGGCACCAGCTCTACCGGTTCTCCA | |
| LCW462_r54 | GGTAGCGAACCGGCAACCTCTGGCTCTGAAACTCCAGGT | GSEPATSGSETPGSEP |
| | AGCGAACCTGCAACCTCCGGCTCTGAAACCCCAGGTACT | ATSGSETPGTSTEPSE |
| | TCTACTGAACCTTCTGAGGGCAGCGCACCAGGTAGCGAA | GSAPGSEPATSGSETP |
| | CCTGCAACCTCTGGCTCTGAAACCCCAGGTACCTCTGAAA | GTSESATPESGPGTST |
| | GCGCTACTCCTGAATCTGGCCCAGGTACTTCTACTGAACC | EPSEGSAPGSSTPSGA |
| | GTCCGAGGGCAGCGCACCAGGTAGCTCTACTCCGTCTGG | TGSPGSSTPSGATGSP |
| | TGCTACCGGCTCTCCAGGTAGCTCTACCCCTTCTGGTGCA | GASPGTSSTGSPGSST |
| | ACCGGCTCCCCAGGTGCTTCTCCGGGTACCAGCTCTACTG | PSGATGSPGASPGTSS |
| | GTTCTCCAGGTAGCTCTACCCCGTCTGGTGCTACCGGTTC | TGSPGSSTPSGATGSP |
| | CCCAGGTGCTTCTCCTGGTACTAGCTCTACCGGTTCTCCA | |
| | GGTAGCTCTACCCCGTCTGGTGCTACTGGCTCTCCA | |
| LCW462_r55 | GGTACTTCTACCGAACCGTCCGAGGGCAGCGCTCCAGGT | GTSTEPSEGSAPGTST |
| | ACTTCTACTGAACCTTCTGAAGGCAGCGCTCCAGGTACTT | EPSEGSAPGTSTEPSE |
| | CTACTGAACCTTCCGAAGGTAGCGCACCAGGTACTTCTGA | GSAPGTSESATPESGP |
| | AAGCGCTACTCCGGAGTCCGGTCCAGGTACCTCTACCGA | GTSTEPSEGSAPGTST |
| | ACCGTCCGAAGGCAGCGCTCCAGGTACTTCTACTGAACCT | EPSEGSAPGSTSESPS |
| | TCTGAGGGTAGCGCTCCAGGTTCTACTAGCGAATCTCCGT | GTAPGTSPSGESSTAP |
| | CTGGCACTGCTCCAGGTACTTCTCCTAGCGGTGAATCTTC | GTSPSGESSTAPGSPA |
| | TACCGCTCCAGGTACTTCCCCTAGCGGCGAATCTTCTACC | GSPTSTEEGTSESATP |
| | GCTCCAGGTAGCCCGGCTGGCTCTCCTACCTCTACTGAGG | ESGPGTSTEPSEGSAP |
| | AAGGTACTTCTGAAAGCGCTACTCCTGAGTCTGGTCCAGG | |
| | TACCTCTACTGAACCGTCCGAAGGTAGCGCTCCA | |
| LCW462_r57 | GGTACTTCTACTGAACCTTCCGAAGGTAGCGCTCCAGGTA | GTSTEPSEGSAPGSEP |
| | GCGAACCTGCTACTTCTGGTTCTGAAACCCCAGGTAGCCC | ATSGSETPGSPAGSPT |
| | GGCTGGCTCTCCGACCTCCACCGAGGAAGGTAGCCCGGC | STEEGSPAGSPTSTEE |
| | AGGCTCTCCGACCTCTACTGAGGAAGGTACTTCTGAAAG | GTSESATPESGPGTST |
| | CGCAACCCCGGAGTCCGGCCCAGGTACCTCTACCGAACC | EPSEGSAPGTSTEPSE |
| | GTCTGAGGGCAGCGCACCAGGTACCTCTACTGAACCTTCC | GSAPGTSTEPSEGSAP |
| | GAAGGCAGCGCTCCAGGTACCTCTACCGAACCGTCCGAG | GTSESATPESGPGSST |
| | GGCAGCGCACCAGGTACTTCTGAAAGCGCAACCCCTGAA | PSGATGSPGSSPSAST |
| | TCCGGTCCAGGTAGCTCTACTCCGTCTGGTGCAACCGGCT | GTGPGASPGTSSTGSP |
| | CCCCAGGTTCTAGCCCGTCTGCTTCCACTGGTACTGGCCC | |
| | AGGTGCTTCCCCGGGCACCAGCTCTACTGGTTCTCCA | |
| LCW462_r61 | GGTAGCGAACCGGCTACTTCCGGCTCTGAGACTCCAGGT | GSEPATSGSETPGSPA |
| | AGCCCTGCTGGCTCTCCGACCTCTACCGAAGAAGGTACCT | GSPTSTEEGTSESATP |
| | CTGAAAGCGCTACCCCTGAGTCTGGCCCAGGTACCTCTAC | ESGPGTSTEPSEGSAP |
| | TGAACCTTCCGAAGGCAGCGCTCCAGGTACCTCTACCGA | GTSTEPSEGSAPGTSE |
| | ACCGTCCGAGGGCAGCGCACCAGGTACTTCTGAAAGCGC | SATPESGPGTSTPESG |
| | AACCCCTGAATCCGGTCCAGGTACCTCTACTCCGGAAAG | SASPGSTSESPSGTAP |
| | CGGTTCCGCATCTCCAGGTTCTACCAGCGAATCCCCGTCT | GSTSSTAESPGPGTSE |
| | GGCACCGCACCAGGTTCTACTAGCTCTACTGCTGAATCTC | SATPESGPGTSTEPSE |
| | CGGGCCCAGGTACTTCTGAAAGCGCTACTCCGGAGTCCG | GSAPGTSTEPSEGSAP |
| | GTCCAGGTACCTCTACCGAACCGTCCGAAGGCAGCGCTC | |
| | CAGGTACTTCTACTGAACCTTCTGAGGGTAGCGCTCCA | |
| LCW462_r64 | GGTACTTCTACCGAACCGTCCGAGGGCAGCGCTCCAGGT | GTSTEPSEGSAPGTST |
| | ACTTCTACTGAACCTTCTGAAGGCAGCGCTCCAGGTACTT | EPSEGSAPGTSTEPSE |
| | CTACTGAACCTTCCGAAGGTAGCGCACCAGGTACCTCTAC | GSAPGTSTEPSEGSAP |
| | CGAACCGTCTGAAGGTAGCGCACCAGGTACCTCTGAAAG | GTSESATPESGPGTSE |
| | CGCAACTCCTGAGTCCGGTCCAGGTACTTCTGAAAGCGC | SATPESGPGTPGSGTA |
| | AACCCCGGAGTCTGGCCCAGGTACTCCTGGCAGCGGTAC | SSSPGSSTPSGATGSP |
| | CGCATCTTCCTCTCCAGGTAGCTCTACTCCGTCTGGTGCA | GASPGTSSTGSPGSTS |
| | ACTGGTTCCCCAGGTGCTTCTCCGGGTACCAGCTCTACCG | STAESPGPGTSPSGES |
| | GTTCTCCAGGTTCCACCAGCTCTACTGCTGAATCTCCTGG | STAPGTSTPESGSASP |
| | TCCAGGTACCTCTCCTAGCGGTGAATCTTCTACTGCTCCA | |
| | GGTACTTCTACTCCTGAAAGCGGCTCTGCTTCTCCA | |
| LCW462_r67 | GGTAGCCCGGCAGGCTCTCCGACCTCTACTGAGGAAGGT | GSPAGSPTSTEEGTSE |
| | ACTTCTGAAAGCGCAACCCCGGAGTCCGGCCCAGGTACC | SATPESGPGTSTEPSE |
| | TCTACCGAACCGTCTGAGGGCAGCGCACCAGGTACTTCT | GSAPGTSESATPESGP |
| | GAAAGCGCAACCCCTGAATCCGGTCCAGGTAGCGAACCG | GSEPATSGSETPGTST |
| | GCTACTTCTGGCTCTGAGACTCCAGGTACTTCTACCGAAC | EPSEGSAPGSPAGSPT |
| | CGTCCGAAGGTAGCGCACCAGGTAGCCCGGCTGGTTCTC | STEEGTSTEPSEGSAP |
| | CGACTTCCACCGAGGAAGGTACCTCTACTGAACCTTCTGA | GTSTEPSEGSAPGTST |
| | GGGTAGCGCTCCAGGTACCTCTACTGAACCTTCCGAAGG | EPSEGSAPGTSTEPSE |
| | CAGCGCTCCAGGTACTTCTACCGAACCGTCCGAGGGCAG | GSAPGTSTEPSEGSAP |
| | CGCTCCAGGTACTTCTACTGAACCTTCTGAAGGCAGCGCT | |
| | CCAGGTACTTCTACTGAACCTTCCGAAGGTAGCGCACCA | |
| LCW462_r69 | GGTACTTCTCCGAGCGGTGAATCTTCTACCGCACCAGGTT | GTSPSGESSTAPGSTS |
| | CTACTAGCTCTACCGCTGAATCTCCGGGCCCAGGTACTTC | STAESPGPGTSPSGES |
| | TCCGAGCGGTGAATCTTCTACTGCTCCAGGTACCTCTGAA | STAPGTSESATPESGP |
| | AGCGCTACTCCGGAGTCTGGCCCAGGTACCTCTACTGAAC | GTSTEPSEGSAPGTST |
| | CGTCTGAGGGTAGCGCTCCAGGTACTTCTACTGAACCGTC | EPSEGSAPGSSPSAST |
| | CGAAGGTAGCGCACCAGGTTCTAGCCCTTCTGCATCTACT | GTGPGSSTPSGATGSP |
| | GGTACTGGCCCAGGTAGCTCTACTCCTTCTGGTGCTACCG | GASPGTSSTGSPGTST |
| | GCTCTCCAGGTGCTTCTCCGGGTACTAGCTCTACCGGTTC | PESGSASPGTSPSGES |
| | TCCAGGTACTTCTACTCCGGAAAGCGGTTCCGCATCTCCA | STAPGTSPSGESSTAP |
| | GGTACTTCTCCTAGCGGTGAATCTTCTACTGCTCCAGGTA | |
| | CCTCTCCTAGCGGCGAATCTTCTACTGCTCCA | |
| LCW462_r70 | GGTACCTCTGAAAGCGCTACTCCGGAGTCTGGCCCAGGT | GTSESATPESGPGTST |
| | ACCTCTACTGAACCGTCTGAGGGTAGCGCTCCAGGTACTT | EPSEGSAPGTSTEPSE |
| | CTACTGAACCGTCCGAAGGTAGCGCACCAGGTAGCCCTG | GSAPGSPAGSPTSTEE |
| | CTGGCTCTCCGACTTCTACTGAGGAAGGTAGCCCGGCTGG | GSPAGSPTSTEEGTST |
| | TTCTCCGACTTCTACTGAGGAAGGTACTTCTACCGAACCT | EPSEGSAPGSSPSAST |
| | TCCGAAGGTAGCGCTCCAGGTTCTAGCCCTTCTGCTTCCA | GTGPGSSTPSGATGSP |
| | CCGGTACTGGCCCAGGTAGCTCTACCCCTTCTGGTGCTAC | GSSTPSGATGSPGSEP |
| | CGGCTCCCCAGGTAGCTCTACTCCTTCTGGTGCAACTGGC | ATSGSETPGTSESATP |
| | TCTCCAGGTAGCGAACCGGCAACTTCCGGCTCTGAAACC | ESGPGSEPATSGSETP |
| | CCAGGTACTTCTGAAAGCGCTACTCCTGAGTCTGGCCCAG | |
| | GTAGCGAACCTGCTACCTCTGGCTCTGAAACCCCA | |
| LCW462_r72 | GGTACTTCTACCGAACCGTCCGAAGGCAGCGCTCCAGGT | GTSTEPSEGSAPGTST |
| | ACCTCTACTGAACCTTCCGAGGGCAGCGCTCCAGGTACCT | EPSEGSAPGTSTEPSE |
| | CTACCGAACCTTCTGAAGGTAGCGCACCAGGTAGCTCTA | GSAPGSSTPSGATGSP |
| | CCCCGTCTGGTGCTACCGGTTCCCCAGGTGCTTCTCCTGG | GASPGTSSTGSPGSST |
| | TACTAGCTCTACCGGTTCTCCAGGTAGCTCTACCCCGTCT | PSGATGSPGTSESATP |
| | GGTGCTACTGGCTCTCCAGGTACTTCTGAAAGCGCAACCC | ESGPGSEPATSGSETP |
| | CTGAATCCGGTCCAGGTAGCGAACCGGCTACTTCTGGCTC | GTSTEPSEGSAPGSTS |
| | TGAGACTCCAGGTACTTCTACCGAACCGTCCGAAGGTAG | ESPSGTAPGSTSESPS |
| | CGCACCAGGTTCTACTAGCGAATCTCCTTCTGGCACTGCA | GTAPGTSTPESGSASP |
| | CCAGGTTCTACCAGCGAATCTCCGTCTGGCACTGCACCAG | |
| | GTACCTCTACCCCTGAAAGCGGTTCCGCTTCTCCA | |
| LCW462_r73 | GGTACCTCTACTCCTGAAAGCGGTTCTGCATCTCCAGGTT | GTSTPESGSASPGSTS |
| | CCACTAGCTCTACCGCAGAATCTCCGGGCCCAGGTTCTAC | STAESPGPGSTSSTAE |
| | TAGCTCTACTGCTGAATCTCCTGGCCCAGGTTCTAGCCCT | SPGPGSSPSASTGTGP |
| | TCTGCATCTACTGGTACTGGCCCAGGTAGCTCTACTCCTT | GSSTPSGATGSPGASP |
| | CTGGTGCTACCGGCTCTCCAGGTGCTTCTCCGGGTACTAG | GTSSTGSPGSEPATSG |
| | CTCTACCGGTTCTCCAGGTAGCGAACCGGCAACCTCCGGC | SETPGTSESATPESGP |
| | TCTGAAACCCCAGGTACCTCTGAAAGCGCTACTCCTGAAT | GSPAGSPTSTEEGSTS |
| | CCGGCCCAGGTAGCCCGGCAGGTTCTCCGACTTCCACTGA | ESPSGTAPGSTSESPS |
| | GGAAGGTTCTACTAGCGAATCTCCTTCTGGCACTGCACCA | GTAPGTSTPESGSASP |
| | GGTTCTACCAGCGAATCTCCGTCTGGCACTGCACCAGGTA | |
| | CCTCTACCCCTGAAAGCGGTTCCGCTTCTCCC | |
| LCW462_r78 | GGTAGCCCGGCTGGCTCTCCTACCTCTACTGAGGAAGGTA | GSPAGSPTSTEEGTSE |
| | CTTCTGAAAGCGCTACTCCTGAGTCTGGTCCAGGTACCTC | SATPESGPGTSTEPSE |
| | TACTGAACCGTCCGAAGGTAGCGCTCCAGGTTCTACCAG | GSAPGSTSESPSGTAP |
| | CGAATCTCCTTCTGGCACCGCTCCAGGTTCTACTAGCGAA | GSTSESPSGTAPGTSP |
| | TCCCCGTCTGGTACCGCACCAGGTACTTCTCCTAGCGGCG | SGESSTAPGTSTEPSE |
| | AATCTTCTACCGCACCAGGTACCTCTACCGAACCTTCCGA | GSAPGSPAGSPTSTEE |
| | AGGTAGCGCTCCAGGTAGCCCGGCAGGTTCTCCTACTTCC | GTSTEPSEGSAPGSEP |
| | ACTGAGGAAGGTACTTCTACCGAACCTTCTGAGGGTAGC | ATSGSETPGTSESATP |
| | GCACCAGGTAGCGAACCTGCAACCTCTGGCTCTGAAACC | ESGPGTSTEPSEGSAP |
| | CCAGGTACCTCTGAAAGCGCTACTCCTGAATCTGGCCCAG | |
| | GTACTTCTACTGAACCGTCCGAGGGCAGCGCACCA | |
| LCW462_r79 | GGTACCTCTACCGAACCTTCCGAAGGTAGCGCTCCAGGT | GTSTEPSEGSAPGSPA |
| | AGCCCGGCAGGTTCTCCTACTTCCACTGAGGAAGGTACTT | GSPTSTEEGTSTEPSE |
| | CTACCGAACCTTCTGAGGGTAGCGCACCAGGTACCTCCCC | GSAPGTSPSGESSTAP |
| | TAGCGGCGAATCTTCTACTGCTCCAGGTACCTCTCCTAGC | GTSPSGESSTAPGTSP |
| | GGCGAATCTTCTACCGCTCCAGGTACCTCCCCTAGCGGTG | SGESSTAPGSTSESPS |
| | AATCTTCTACCGCACCAGGTTCTACCAGCGAATCCCCTTC | GTAPGSTSESPSGTAP |
| | TGGTACTGCTCCAGGTTCTACCAGCGAATCCCCTTCTGGC | GTSTPESGSASPGSEP |
| | ACCGCACCAGGTACTTCTACCCCTGAAAGCGGCTCCGCTT | ATSGSETPGTSESATP |
| | CTCCAGGTAGCGAACCTGCAACCTCTGGCTCTGAAACCCC | ESGPGTSTEPSEGSAP |
| | AGGTACCTCTGAAAGCGCTACTCCTGAATCTGGCCCAGGT | |
| | ACTTCTACTGAACCGTCCGAGGGCAGCGCACCA | |
| LCW462_r87 | GGTAGCGAACCGGCAACCTCTGGCTCTGAAACCCCAGGT | GSEPATSGSETPGTSE |
| | ACCTCTGAAAGCGCTACTCCGGAATCTGGTCCAGGTACTT | SATPESGPGTSESATP |
| | CTGAAAGCGCTACTCCGGAATCCGGTCCAGGTACTTCTCC | ESGPGTSPSGESSTAP |
| | GAGCGGTGAATCTTCTACCGCACCAGGTTCTACTAGCTCT | GSTSSTAESPGPGTSP |
| | ACCGCTGAATCTCCGGGCCCAGGTACTTCTCCGAGCGGTG | SGESSTAPGSTSESPS |
| | AATCTTCTACTGCTCCAGGTTCTACTAGCGAATCCCCGTC | GTAPGTSPSGESSTAP |
| | TGGTACTGCTCCAGGTACTTCCCCTAGCGGTGAATCTTCT | GSTSSTAESPGPGSST |
| | ACTGCTCCAGGTTCTACCAGCTCTACCGCAGAATCTCCGG | PSGATGSPGSSTPSGA |
| | GTCCAGGTAGCTCTACTCCGTCTGGTGCAACCGGTTCCCC | TGSPGSSTPSGANWLS |
| | AGGTAGCTCTACCCCTTCTGGTGCAACCGGCTCCCCAGGT | |
| | AGCTCTACCCCTTCTGGTGCAAACTGGCTCTCC | |
| LCW462_r88 | GGTAGCCCTGCTGGCTCTCCGACTTCTACTGAGGAAGGTA | GSPAGSPTSTEEGSPA |
| | GCCCGGCTGGTTCTCCGACTTCTACTGAGGAAGGTACTTC | GSPTSTEEGTSTEPSE |
| | TACCGAACCTTCCGAAGGTAGCGCTCCAGGTACCTCTACT | GSAPGTSTEPSEGSAP |
| | GAACCTTCCGAAGGCAGCGCTCCAGGTACCTCTACCGAA | GTSTEPSEGSAPGTSE |
| | CCGTCCGAGGGCAGCGCACCAGGTACTTCTGAAAGCGCA | SATPESGPGASPGTSS |
| | ACCCCTGAATCCGGTCCAGGTGCATCTCCTGGTACCAGCT | TGSPGSSTPSGATGSP |
| | CTACCGGTTCTCCAGGTAGCTCTACTCCTTCTGGTGCTAC | GASPGTSSTGSPGSST |
| | TGGCTCTCCAGGTGCTTCCCCGGGTACCAGCTCTACCGGT | PSGATGSPGTPGSGT |
| | TCTCCAGGTAGCTCTACCCCGTCTGGTGCTACTGGTTCTC | ASSSPGSSTPSGATGSP |
| | CAGGTACTCCGGGCAGCGGTACTGCTTCTTCCTCTCCAGG | |
| | TAGCTCTACCCCTTCTGGTGCTACTGGCTCTCCA | |
| LCW462_r89 | GGTAGCTCTACCCCGTCTGGTGCTACTGGTTCTCCAGGTA | GSSTPSGATGSPGTPG |
| | CTCCGGGCAGCGGTACTGCTTCTTCCTCTCCAGGTAGCTC | SGTASSSPGSSTPSGA |
| | TACCCCTTCTGGTGCTACTGGCTCTCCAGGTAGCCCGGCT | TGSPGSPAGSPTSTEE |
| | GGCTCTCCTACCTCTACTGAGGAAGGTACTTCTGAAAGCG | GTSESATPESGPGTST |
| | CTACTCCTGAGTCTGGTCCAGGTACCTCTACTGAACCGTC | EPSEGSAPGTSESATP |
| | CGAAGGTAGCGCTCCAGGTACCTCTGAAAGCGCAACTCC | ESGPGSEPATSGSETP |
| | TGAGTCTGGCCCAGGTAGCGAACCTGCTACCTCCGGCTCT | GTSESATPESGPGTST |
| | GAGACTCCAGGTACCTCTGAAAGCGCAACCCCGGAATCT | EPSEGSAPGTSESATP |
| | GGTCCAGGTACTTCTACTGAACCGTCTGAAGGTAGCGCA | ESGPGTSESATPESGP |
| | CCAGGTACTTCTGAAAGCGCAACCCCGGAATCCGGCCCA | |
| | GGTACCTCTGAAAGCGCAACCCCGGAGTCCGGCCCA | |

### Example 7

### Construction of XTEN_AM288

The entire library LCW0462 was dimerized as described in Example 6 resulting in a library of XTEN_AM288 clones designated LCW0463. 1512 isolates from library LCW0463 were screened using the protocol described in Example 6. 176 highly expressing clones were sequenced and 40 preferred XTEN_AM288 segments were chosen for the construction of multifunctional proteins that contain multiple XTEN segments with 288 amino acid residues.

### Example 8

### Construction of XTEN_AM432

We generated a library of XTEN_AM432 segments by recombining segments from library LCW0462 of XTEN_AM144 segments and segments from library LCW0463 of XTEN_AM288 segments. This new library of XTEN_AM432 segment was designated LCW0464. Plasmid was isolated from cultures of E. coli harboring LCW0462 and LCW0463, respectively. 1512 isolates from library LCW0464 were screened using the protocol described in Example 6. 176 highly expressing clones were sequenced and 39 preferred XTEN_AM432 segment were chosen for the construction of longer XTENs and for the construction of multifunctional proteins that contain multiple XTEN segments with 432 amino acid residues.

In parallel we constructed library LMS0100 of XTEN_AM432 segments using preferred segments of XTEN_AM144 and XTEN_AM288. Screening of this library yielded 4 isolates that were selected for further construction

### Example 9

### Construction of XTEN_AM875

The stuffer vector pCW0359 was digested with BsaI and KpnI to remove the stuffer segment and the resulting vector fragment was isolated by agarose gel purification.

We annealed the phosphorylated oligonucleotide “BsaI-AscI-KpnIforP”: AGGTGCAAGCGCAAGCGGCGCGCCAAGCACGGGAGGTTCGTCTTCACTCGAGGGTAC and the non-phosphorylated oligonucleotide “BsaI-AscI-KpnIrev”: CCTCGAGTGAAGACGAACCTCCCGTGCTTGGCGCGCCGCTTGCGCTTGC for introducing the sequencing island A (SI-A) which encodes amino acids GASASGAPSTG and has the restriction enzyme AscI recognition nucleotide sequence GGCGCGCC inside. The annealed oligonucleotide pairs were ligated with BsaI and KpnI digested stuffer vector pCW0359 prepared above to yield pCW0466 containing SI-A. We then generated a library of XTEN_AM443 segments by recombining 43 preferred XTEN_AM432 segments from Example 8 and SI-A segments from pCWO466 at C-terminus using the same dimerization process described in Example 5. This new library of XTEN_AM443 segments was designated LCW0479.

We generated a library of XTEN_AM875 segments by recombining segments from library LCW0479 of XTEN_AM443 segments and 43 preferred XTEN_AM432 segments from Example 8 using the same dimerization process described in Example 5. This new library of XTEN_AM875 segment was designated LCW0481.

### Example 10

### Construction of XTEN_AM1318

We annealed the phosphorylated oligonucleotide “BsaI-FseI-KpnIforP”: AGGTCCAGAACCAACGGGGCCGGCCCCAAGCGGAGGTTCGTCTTCACTCGAGGGTAC and the non-phosphorylated oligonucleotide “BsaI-FseI-KpnIrev”: CCTCGAGTGAAGACGAACCTCCGCTTGGGGCCGGCCCCGTTGGTTCTGG for introducing the sequencing island B (SI-B) which encodes amino acids GPEPTGPAPSG and has the restriction enzyme FseI recognition nucleotide sequence GGCCGGCC inside. The annealed oligonucleotide pairs were ligated with BsaI and KpnI digested stuffer vector pCW0359 as used in Example 9 to yield pCWO467 containing SI-B. We then generated a library of XTEN_AM443 segments by recombining 43 preferred XTEN_AM432 segments from Example 8 and SI-B segments from pCWO467 at C-terminus using the same dimerization process described in Example 5. This new library of XTEN_AM443 segments was designated LCW0480.

We generated a library of XTEN_AM1318 segments by recombining segments from library LCW0480 of XTEN_AM443 segments and segments from library LCW0481 of XTEN_AM875 segments using the same dimerization process as in Example 5. This new library of XTEN_AM1318 segment was designated LCW0487.

### Example 11

### Construction of XTEN_AD864

Using the several consecutive rounds of dimerization, we assembled a collection of XTEN_AD864 sequences starting from segments of XTEN_AD36 listed in Example 1. These sequences were assembled as described in Example 5. Several isolates from XTEN_AD864 were evaluated and found to show good expression and excellent solubility under physiological conditions. One intermediate construct of XTEN_AD576 was sequenced. This clone was evaluated in a PK experiment in cynomolgus monkeys and a half-life of about 20 h was measured.

### Example 12

### Construction of XTEN_AF864

Using the several consecutive rounds of dimerization, we assembled a collection of XTEN_AF864 sequences starting from segments of XTEN_AF36 listed in Example 3. These sequences were assembled as described in Example 5. Several isolates from XTEN_AF864 were evaluated and found to show good expression and excellent solubility under physiological conditions. One inter mediate construct of XTEN_AF540 was sequenced. This clone was evaluated in a PK experiment in cynomolgus monkeys and a half-life of about 20 h was measured. A full length clone of XTEN_AF864 had excellent solubility and showed half-life exceeding 60 h in cynomolgus monkeys. A second set of XTEN_AF sequences was assembled including a sequencing island as described in Example 9.

### Example 13

### Construction of XTEN_AG864

Using the several consecutive rounds of dimerization, we assembled a collection of XTEN_AG864 sequences starting from segments of XTEN_AG36 listed in Example 4. These sequences were assembled as described in Example 5. Several isolates from XTEN_AG864 were evaluated and found to show good expression and excellent solubility under physiological conditions. A full-length clone of XTEN_AG864 had excellent solubility and showed half-life exceeding 60 h in cynomolgus monkeys.

### Example 14

### Methods of Producing and Evaluating CFXTEN with Internal and Terminal XTEN

The design, construction and evaluation of CFXTEN comprising FVIII and one or more XTEN is accomplished using a systematic approach. The regions suitable for XTEN insertion sites include, but are to limited to regions at or proximal to the known domain boundaries of FVIII, exon boundaries, known surface loops, regions with a low degree of order, and hydrophilic regions. By analysis of the foregoing, different regions across the sequence of the FVIII B domain deleted (BDD) sequence have been identified as insertion sites for XTEN, non-limiting examples of which are listed in Tables 5 and 25, and shown schematically in FIGS. 6 and 7. Individual constructs are created (using methods described, below) in which DNA encoding a single XTEN or XTEN fragment of a length ranging from 6 to 2004 amino acid residues is inserted into the FVIII sequence corresponding to or near (e.g., within 6 amino acids) each of the single insertion sites identified in Table 5 and Table 25, and the resulting constructs are expressed and the recovered protein then evaluated for their effects on retention of procoagulant activity using, e.g., one of the in vitro assays of Table 27. For example, using the methods described below, constructs are made in which an AG42 sequence is inserted between the A1 and A2 domain sequences of FVIII, and the resulting expressed fusion protein is evaluated in a chromogenix assay of Table 27, compared to a FVIII not linked to XTEN. CFXTEN fusion proteins can be further classified acting to high, intermediate and low categories based on the activities they exhibit. In those cases where the CFXTEN exhibits activity that is comparable or modestly reduced compared to FVIII, the insertion site is deemed favorable. In those cases where the activity is intermediate, the insertion site can be adjusted from 1-6 amino acids towards the N- or C-terminus of the insertion site and/or the length or net charge of the XTEN may be altered and the resulting construct(s) re-evaluated to determine whether the activity is improved. Alternatively, the XTEN is inserted into the construct with flanking cleavage sites; preferably sites that are susceptible to cleavage by proteases found in clotting assays, such that the XTEN is released during the activation of the FVIII component, thereby providing additional information about the suitability of the XTEN insertion site in the fusion protein.

Once all of the individual insertion sites are evaluated and the favorable insertion sites are identified, constructs are created with two, three, four, five or more XTEN inserted in the favorable sites. The length and net charge of the XTEN (e.g., XTEN of the AE versus AG family) are varied in order to ascertain the effects of these variables on FVIII activity and physicochemical properties of the fusion protein. CFXTEN constructs that retain a desired degree of in vitro FVIII activity are then evaluated in vivo using mouse and/or dog models of hemophilia A, as described in Examples below, or other models known in the art. In addition, CFXTEN constructs are made that incorporate cleavage sequences at or near the junction(s) of FVIII and XTEN (e.g., sequences from Table 7) designed to release the XTEN and are evaluated for enhancement of FVIII activity and effects on terminal half-life. By the iterative process of making constructs combining different insertion sites, varying the length and composition qualities of the XTEN (e.g., different XTEN families), and evaluation, the skilled artisan obtains, by the foregoing methods, CFXTEN with desired properties, such as but not limited to of procoagulant FVIII activity, enhanced pharmacokinetic properties, ability to administer to a subject by different routes, and/or enhanced pharmaceutical properties.

### Example 15

### Methods of Producing and Evaluating CFXTEN Containing FVIII and AE_XTEN

A general scheme for producing and evaluating CFXTEN compositions is presented in FIG. 13, and folios the basis for the general description of this Example. Using the disclosed methods and those known to one of ordinary skill in the art, together with guidance provided in the illustrative examples, a skilled artesian can create and evaluate CFXTEN fusion proteins comprising XTEN and FVIII or variants of FVIII known in the art. The Example is, therefore, to be construed as merely illustrative, and not limitative of the methods in any way whatsoever; numerous variations will be apparent to the ordinarily skilled artisan. In this Example, a CFXTEN of a factor VIII BDD linked to an XTEN of the AE family of motifs is created.

The general scheme for producing polynucleotides encoding XTEN is presented in FIGS. 11 and 12. FIG. 12 is a schematic flowchart of representative steps in the assembly of an XTEN polynucleotide construct in one of the embodiments of the invention. Individual oligonucleotides 501 are annealed into sequence motifs 502 such as a 12-amino acid motif (“12-mer”), which is ligated to additional sequence motifs from a library that can multimerize to create a pool that encompasses the desired length of the XTEN 504, as well as ligated to a smaller concentration of an oligo containing BbsI, and KpnI restriction sites 503. The motif libraries include specific sequence XTEN families; e.g., AD, AE, AF, AG, AM, or AQ sequences of Table 3. As illustrated in FIG. 12, the XTEN length, in this case, is 36 amino acid residues, but longer lengths are also achieved by this general process. For example, multimerization is performed by ligation, overlap extension, PCR assembly or similar cloning techniques known in the art. The resulting pool of ligation products is gel-purified and the band with the desired length of XTEN is cut, resulting in an isolated XTEN gene with a stopper sequence 505. The XTEN gene can be cloned into a stuffer vector. In this case, the vector encodes an optional CBD sequence 506 and a GFP gene 508. Digestion is then performed with BbsI/HindIII to remove 507 and 508 and place the stop codon. The resulting product is then cloned into a BsaI/HindIII digested vector containing a gene encoding the FVIII, resulting in the gene 500 encoding a CFXTEN fusion protein. As would be apparent to one of ordinary skill in the art, the methods are applied to create constructs in alternative configurations and with varying XTEN lengths.

DNA sequences encoding FVIII are conveniently obtained by standard procedures known in the art from a cDNA library prepared from an appropriate cellular source, from a genomic library, or may be created synthetically (e.g., automated nucleic acid synthesis) using DNA sequences obtained from publicly available databases, patents, or literature references. In the present example, a FVIII B domain deleted (BDD) variant is prepared as described in Example 17. A gene or polynucleotide encoding the FVIII portion of the protein or its complement is then cloned into a construct, such as those described herein, which can be a plasmid or other vector under control of appropriate transcription and translation sequences for high level protein expression in a biological system. A second gene or polynucleotide coding for the XTEN portion or its complement is genetically fused to the nucleotides encoding the terminus of the FVIII gene by cloning it into the construct adjacent and in frame with the gene coding for the CF, through a ligation or multimerization step. In this manner, a chimeric DNA molecule coding for (or complementary to) the CFXTEN fusion protein is generated within the construct. Optionally, a gene encoding for a second XTEN is inserted and ligated in-frame internally to the nucleotides encoding the FVIII-encoding region. The constructs are designed in different configurations to encode various insertion sites of the XTEN in the FVIII sequence, including those of Table 5 or Table 25 or as illustrated in FIG. 7. Optionally, this chimeric DNA molecule is transferred or cloned into another construct that is a more appropriate expression vector; e.g., a vector appropriate for a mammalian host cell such as CHO, BHK and the like. At this point, a host cell capable of expressing the chimeric DNA molecule is transformed with the chimeric DNA molecule, described more completely, below, or by well-known methods, depending on the type of cellular host, as described supra.

Host cells containing the XTEN-FVIII expression vector are cultured in conventional nutrient media modified as appropriate for activating the promoter. The culture conditions, such as temperature, pH and the like, are those previously used with the host cell selected for expression, and will be apparent to the ordinarily skilled artisan. After expression of the fusion protein, culture broth is harvested and separated from the cell mass and the resulting crude extract retained for purification of the fusion protein.

Gene expression is measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, gene expression is measured by immunological of fluorescent methods, such as immunohistochemical staining of cells to quantitate directly the expression of gene product. Antibodies useful for immunohistochemical staining and/or assay of sample fluids may be either monoclonal or polyclonal, and may be prepared in any mammal. Conveniently, the antibodies may be prepared against the FVIII sequence polypeptide using a synthetic peptide based on the sequences provided herein or against exogenous sequence fused to FVIII and encoding a specific antibody epitope. Examples of selectable markers are well known to one of skill in the art and include reporters such as enhanced green fluorescent protein (EGFP), beta-galactosidase (β-gal) or chloramphenicol acetyltransferase (CAT).

The CFXTEN polypeptide product is purified via methods known in the art. Procedures such as gel filtration, affinity purification, salt fractionation, ion exchange chromatography, size exclusion chromatography, hydroxyapatite adsorption chromatography, hydrophobic interaction chromatography or gel electrophoresis are all techniques that may be used in the purification. Specific methods of purification are described in Robert K. Scopes, Protein Purification: Principles and Practice, Charles R. Castor, ed., Springer-Verlag 1994, and Sambrook, et al., supra. Multi-step purification separations are also described in Baron, et al., Crit. Rev. Biotechnol. 10:179-90 (1990) and Below, et al., J. Chromatogr. A. 679:67-83 (1994).

As illustrated in FIG. 13, the isolated CFXTEN fusion proteins are characterized for their chemical and activity properties. An isolated fusion protein is characterized, e.g., for sequence, purity, apparent molecular weight, solubility and stability using standard methods known in the art. The fusion protein meeting expected standards is evaluated for activity, which can be measured in vitro or in vivo by measuring one of the factor VIII-associated parameters described herein, using one or more assays disclosed herein, or using the assays of the Examples or Table 27.

In addition, the CFXTEN FVIII fusion protein is administered to one or more animal species to determine standard pharmacokinetic parameters and pharmacodynamic properties, as described in Examples 25 and 26.

By the iterative process of producing, expressing, and recovering CFXTEN constructs, followed by their characterization using methods disclosed herein or others known in the art, the CFXTEN compositions comprising CF and an XTEN are produced and evaluated to confirm the expected properties such as enhanced solubility, enhanced stability, improved pharmacokinetics and reduced immunogenicity, leading to an overall enhanced therapeutic activity compared to the corresponding unfused FVIII. For those fusion proteins not possessing the desired properties, a different sequence or configuration is constructed, expressed, isolated and evaluated by these methods in order to obtain a composition with such properties.

### Example 16

### Construction of Expression Plasmids for BDD FVIII

I. Construction of B Domain Deleted FVIII (BDD FVIII) Expression Vectors

The expression vector encoding BDD FVIII was created by cloning the BDD FVIII open reading frame into the pcDNA4 vector (Invitrogen, CA) containing a polyA to allow for optimal mammalian expression of the FVIII gene, resulting in a construct designated pBC0100. Several natural sites were identified within this construct for cloning use, including BsiWI 48, AfIII 381, PshAI 1098, KpnI 1873, BamHI 1931, PflMI 3094, ApaI 3574, XbaI 4325, NotI 4437, XhoI 4444, BstEII 4449, AgeI 4500, PmeI 4527. To facilitate assay development, nucleotides encoding Myc and His tag were introduced into the FVIII open reading frame. pBC0100 was PCR amplified using the following primers: 1) F8-BsiWI-F: tattccCGTACGgccgccaccATGCAAATAGAGCTCTCCACCT; 2) F8-nostop-XhoI-R1: GGTGACCTCGAGcgtagaggtectgtgcctcg to introduce BsiWI and XhoI in appropriate locations. The PCR product was digested with BsiWI and XhoI. PcDNA4-Myc-His/C was digested with Acc65I and XhoI, which generated two products of 5003 and 68 bps. The 5003 bps product was ligated with the digested PCR'ed FVIII fragment and used for DH5alpha transformation. The enzymes Acc65I and BsiWI create compatible ends but this ligation destroys the site for future digestion. The resulting construct was designated pBC0102 (pcDNA4-FVIII_{—}3-Myc-His). To facilitate the design and execution of future cloning strategies, especially ones involving the creation of BDD FVIII expression constructs that contain multiple XTEN insertions, we selected additional unique restriction enzyme sites to incorporate, including BsiWI 908, NheI 1829 and ClaI 3281. The introduction of these sites was done via the QuikChange method (Agilent, CA) individually. The resulting construct was designated pBC0112 (pcDNA4-FVIII_{—}4-Myc-His). To avoid problems that may arise from the linker peptides that connects between Myc/His and FVIII/Myc, and to remove restriction enzyme sites that are preferred for future XTEN insertion, we mutated the sequences encoding the peptide sequences from ARGHPF to GAGSPGAETA (between FVIII and Myc), NMHTG to SPATG (between Myc and His) via the QuikChange method. The construct was designated pBC0114 (pcDNA4-FVIII_{—}4-GAGSPGAETA-Myc-SPATG-His) (sequence in Table 14), which was used as the base vector for the design and creation of other expression vectors incorporating XTEN sequences. Expression and FVIII activity data for this construct are presented in

II. Construction of B Domain Deleted FVIII (BDD FVIII) Expression Vectors

The gene encoding BDD FVIII is synthesized by GeneArts (Regensburg, Germany) in the cloning vector pMK (pMK-BDD FVIII). The BDD FVIII proteins contain 1457 amino acids at a total molecular weight of 167539.66. There are 6 domains within the wild-type FVIII protein, the A1, A2, B, A3, C1 and C2 domains. In the BDD FVIII protein, most of the B domain has been deleted as it was shown to be an unstructured domain and the removal of the domain does not alter critical functions of this protein. The pMK vector used by GeneArts contains no promoter, and can not be used as an expression vector. Restriction enzyme sites NheI on the 5′ end and SfiI, SalI and XhoI on the 3′ end are introduced to facilitate subcloning of the DNA sequence encoding BDD FVIII into expression vectors, such as CET1019-HS (Millipore). Several unique restriction enzyme sites are also introduced into the FVIII sequence to allow further manipulation (e.g., insertion, mutagenesis) of the DNA sequences. Unique sites listed with their cut site include, but are not limited to: SacI 391, AfiII 700, SpeI 966, PshAI 1417, Acc65I 2192, KpnI 2192, BamHI 2250, HindIII 2658, PfoI 2960, PflMI 3413, ApaI 3893, Bsp1201 3893, SwaI 4265, OliI 4626, XbaI 4644, and BstBI 4673. The HindIII site resides at the very end of the A2 domain and can potentially be used for modification of the B domain. The synthesized pMK-BDD FVIII from GeneArts does not contain a stop codon. The stop codon is introduced by amplifying a 127 bp fragment of FVIII using the following primers: 5′-GTGAACTCTCTAGACCCACCG-3′; 5′-CTCCTCGAGGTCGACTCAGTAGAGGTCCTGTGCCTCG-3′. The fragment is digested with XbaI and SalI, and ligated to XbaI/SalI digested pMK-BDD FVIII. The ligated DNA mixture is used to transform DH5a bacterial cells. Transformants are screened by DNA miniprep and the desired constructs are confirmed by DNA sequencing. The construct named pBC0027 (pMK-BDD FVIII-STOP) contains coding sequences that encode the BDD FVIII protein. The pBC0027 construct is then digested with NheI/SalI, and ligated with NheI/SalI digested CET1019-HS vector (Millipore). The CET1019-HS vector contains a human CMV promoter and a UCOE sequence to facilitate gene expression. The ligated DNA mixture is used to transform DH5a bacterial cells. Transformants are screened by DNA miniprep and the desired constructs are confirmed by DNA sequencing. The final construct is designated pBC0025 (CET1019-HS-BDD FVIII-STOP), which encodes the BDD FVIII protein under the control of a human CMV promoter. Introduction of the pBC0025 construct into mammalian cells is expected to allow expression of the BDD FVIII protein with procoagulant activity.

### Example 17

### Construction of Expression Plasmids for BDD FVIII Containing XTEN

1. B domain AE42 Insertion

Two PCR reactions were run to in parallel to insert XTEN_AE42 into the remaining B domain region of the BDD FVIII constructs. The PCR reactions involved the following primers: cgaaagcgctacgcctgagaGTGGCCCTGGCTCTGAGCCAGCCACCTCCGGCTCTGAAACCCCTGCCTCG AGCccaccagtcttgaaacgcc; TGATATGGTATCATCATAATCGATTTCCTCTTGATCTGACTG; agcttgaggatccagagttc; tctcaggcgtagcgctttcgCTTGTCCCCTCTTCTGTTGAGGTGGGGGAGCCAGCAGGAGAACCTGGCGCG CCgttttgagagaagcttcttggt. The PCR products then served as templates, and a second PCR was performed to introduce the XTEN_AE42 into the FVIII encoding nucleotide sequences flanked by BamHI and ClaI. This PCR product was digested with BamHI and ClaI simultaneously with the digestion of PBC0114 with the same two enzymes. The PCR product was ligated to the digested vector. This construct was designated pBC0135 (pcDNA4-FVIII_{—}4XTEN_AE42-GAGSPGAETA-Myc-SPATG-His), and encodes the BDD FVIII with an AE42 XTEN incorporated within the residual B-domain.

2. AE42 Insertion and R1648A Mutation

The QuikChange method (Agilent, CA) was employed to introduce an R1648A mutation into PBC0135. This construct was designated pBC0149 (pcDNA4-FVIII_{—}4XTEN_AE42-GAGSPGAETA-Myc-SPATG-His_R1648A), eliminating that FVIII processing site.

3. B domain AE288 Insertion

XTEN_AE288 was PCR amplified using the following primers: tctcaaaacGGCGCGCCAggtacctcagagtctgctacc and tggtggGCTCGAGGCtggcgcactgectte. PBC0075 was used as the template for this PCR reaction. The PCR product was digested with AscI and XhoI, and PBC0135 was digested with the same enzymes. The PCR product was ligated to the PBC0135 fragment. This construct was designated pBC0136 (pcDNA4-FVIII_{—}4XTEN_AE288-GAGSPGAETA-Myc-SPATG-His), and encodes the BDD FVIII with an AE288 XTEN incorporated within the residual B-domain.

4. AE288 Insertion and R1648A Mutation

XTEN_AE288 was PCR amplified using the following primers: tctcaaaacGGCGCGCCAggtacctcagagtctgctacc and tggtggGCTCGAGGCtggcgcactgccttc. Construct pBC0075 was used as the template for this PCR reaction. The PCR product was digested with AscI and XhoI, and pBC0149 was digested with the same enzymes. The PCR product was ligated to the pBC0149 fragment. This construct was designated pBC0137 (pcDNA4-FVIII_{—}4XTEN_AE288-GAGSPGAETA-Myc-SPATG-His R1648A) and contains an AE288 XTEN sequence internal to the B domain, with the R1648A mutation eliminating that FVIII processing site.

Construction of Expression Plasmids for BDD FVIII with XTEN Insertion at the C Terminus

1. C Terminal AE288 Insertion

XTEN_AE288 was PCR amplified using the following primers: ggggccgaaacggccggtacctcagagtctgctacc and tgttcggccgtttcggcccctggcgcactgccttc. The construct pBC0075 was used as the template for this PCR reaction. The PCR product was digested with SfiI, and pBC0114 was digested with the same enzyme. The PCR product was ligated to the digested pBC0114 fragment. This construct was designated pBC0145 (pcDNA4-FVIII_{—}4-XTEN_AE288-GAGSPGAETA-Myc-SPATG-His), and encodes an AE288 sequence at the C-terminus of the BDD

2. C Terminal AG288 Insertion

XTEN_AG288 was designed and synthesized by DNA2.0 (Menlo Park, Calif.). The synthesized gene was PCR amplified using the following primers: ggggccgaaacggccccgggagcgtcacc and tgttcggccgtttcggcccctgacccggttgcccc. The PCR product was digested with SfiI, and PBC0114 based vector was digested with the same enzyme. The PCR product was ligated to the digested PBC0114 fragment. This construct was designated pBC0146 (pcDNA4-FVIII_{—}4-XTEN_AG288-GAGSPGAETA-Myc-SPATG-His), and encodes an AG288 sequence at the C-terminus of the BDD FVIII.

Construction of Expression Plasmids for BDD FVIII with Inter- and Intra-Domain XTEN Insertions

1. AE42 Insertion

Four distinct strategies are used for insertion of AE42 into the designated sites (e.g., the natural or introduced restriction sites BsiWI 48, AflII 381, PshAI 1098, KpnI 1873, BamHI 1931, PflMI 3094, ApaI 3574, XbaI 4325, NotI 4437, XhoI 4444, BstEII 4449, AgeI 4500, PmeI 4527, BsiWI 908, NheI 1829 and ClaI 3281) within the BDD FVIII encoding sequence, each contributing to the creation of several constructs. By design, these insertions of AE42 create AscI and XhoI sites flanked on either side of the insertion allowing for introduction/substitution of longer XTEN, as well as XTEN with different sequences or incorporated cleavage sequences, as needed.

2. Double PCR-Mediated Method

Two PCR reactions are run in parallel to insert XTEN_AE42 into the designated site. The two PCR reactions introduce XTEN on either the 3′ or the 5′ end via use of a long primer that contains partial XTEN. The PCR products then serve as templates, and a second PCR is performed to introduce the XTEN_AE42 into the FVIII encoding nucleotide sequences flanked by select restriction enzyme sites. This PCR product is digested with the appropriate enzymes simultaneously with the digestion of PBC0114 using the same two enzymes. The PCR product is ligated to the digested vector. Using this method, constructs are created designated pBC0126, pBC0127, pBC0128, and pBC0129, resulting in AE42 insertions at the R3, P130, L216 locations. The sequences are listed in Table 14.

3. QuikChange Mediated Two Step Cloning Method

The QuikChange method is employed to introduce XTEN_AE7 encoding sequences that are flanked by AscI and XhoI into designated sites. The resulting intermediate construct is then digested with AscI and XhoI. XTEN_AE42 is PCR amplified to introduce the two sites and digested accordingly. The vector and insert are then ligated to create the final constructs, designated pBC0131, pBC0134, pBC0138, pBC0141, pBC0142 and pBC0143, suitable for allowing introduction of longer XTEN, as well as XTEN with different sequences or incorporated cleavage sequences, as needed. The sequences are listed in Table 14.

4. Three PCR Type II Restriction Enzyme Mediated Ligation Method

Three PCR reactions are performed to create two pieces of FVIII encoding fragments flanked by one type I restriction enzyme that correlates with a unique site within the FVIII_{—}4 gene and one type II enzyme (e.g. BsaI, BbsI, BfuAI), the third PCR reaction created the XTEN_AE42 flanked by two type II restriction enzyme sites. The three PCR fragments are digested with appropriate enzymes and ligated into one linear piece that contains the XTEN_AE42 insertion within a fragment of FVIII encoding sequences. This product is then digested with appropriate unique enzymes within the FVIII encoding sequences and ligated to the PBC0114 construct digested with the same enzymes, and result in constructs designated pBC0130 (with XTEN insertion at residue P333), pBC0132 (with XTEN insertion at residue D403), pBC0133 (with XTEN insertion at residue R490). The sequences are listed in Table 14.

5. Custom Gene Synthesis

Custom gene synthesis is performed by GeneArt (Regensburg, Germany). The genes are designed so that they include nucleotides encoding the XTEN_AE42 inserted in the designated site(s) and the genes are flanked by two unique restriction enzyme sites selected within the FVIII_{—}4 gene. The synthesized genes and PBC0114 are digested with appropriate enzymes and ligated to create the final product with the BDD FVIII incorporating the XTEN_AE42 between the restriction sites. All constructs not listed in above strategies are constructed based on this method.

Construction of Expression Plasmids with Dual XTEN Insertions in the B Domain and at the C Terminus

The construct pBC0136, which encodes the BDD FVIII with an AE288 XTEN incorporated within the residual B-domain, is digested with BamHI and ClaI, and the resulting 1372 bps fragment from this digestion is the insert. The construct pBC0146 is digested with BamHI and ClaI, and the 9791 bps piece from this digestion is the vector. The vector and insert are ligated together to create pBC0209, containing an AE288 insertion within the B domain and an AG288 on the C terminus. The same strategy is utilized to create constructs containing two AE288 insertions in the B domain and at the C terminus, respectively, using PBC0145 as the vector.

Construction of Expression Plasmids with Multiple XTEN Insertions

The construct pBC0127, which encodes an AE42 XTEN at the R3 position of FVIII, is digested with BsiWI and AflII, and the resulting 468 bps fragment from this digestion is the insert. The construct pBC0209 is digested with BsiWI and AflII, the 10830 bps piece from this digestion is the vector. The vector and insert are ligated together to create a construct designated pBC0210, containing an AE42 insertion in the A1 domain, an extra three ATR amino acid to restore the signal cleavage sequence, an AE288 XTEN insertion within the B domain and an AG288 on the C terminus. The same methodology is used to create constructs encoding multiple XTEN at the natural and introduced restriction sites; e.g., BsiWI 48, AflII 381, PshAI 1098, KpnI 1873, BamHI 1931, PflMI 3094, ApaI 3574, XbaI 4325, NotI 4437, XhoI 4444, BstEII 4449, AgeI 4500, PmeI 4527, BsiWI 908, NheI 1829 and ClaI 3281.

Construction of BDD FVIII-INTERNAL-XTEN_AE288 Expression Vectors

Two BsaI restriction enzyme sites are introduced into the PBC0027 pMK-BDD FVIII construct between the base pair 2673 and 2674 using the QuikChange method following manufacturer's protocol (Agilent Technologies, CA). The inserted DNA sequences are gggtctcccgcgccagggtctccc, and the resulting construct is designated pBC0205 (sequence in Table 14). The DNA sequence encoding AE288 (or other variants and lengths of XTEN; e.g. AE42, AG42, AG288, AM288) is then PCR'ed with primers that introduce BsaI sites on both the 5′ and 3′. The pBC0205 vector and the insert (XTEN_{—}288) are then digested with BsaI and ligated to create pBC0206, which encodes the FVIII gene with an XTEN_AE288 insertion within the B domain (sequence in Table 14). The pBC0206 construct is then digested with NheI/SalI, and ligated with NheI/SalI digested CET1019-HS vector (Millipore). The CET 1019-HS vector contains a human CMV promoter and a UCOE sequence to facilitate gene expression. The ligated DNA mixture is used to transform DH5a bacterial cells. Transformants are screened by DNA miniprep and the desired constructs are confirmed by DNA sequencing. The final construct is designated pBC0207 (CET1019-HS-BDD FVIII-STOP), which encodes the BDD FVIII protein under the control of a human CMV promoter (sequence in Table 14). Introduction of the pBC0207 construct into mammalian cells is expected to allow expression of the BDD FVIII protein with an internal XTEN_AE288. The same protocol is used to introduce, transform and express constructs containing other variants and lengths of XTEN; e.g. AE42, AG42, AG288, AM288, AE864, AG864, or other XTEN of Table 4.

Construction of BDD FVIII−/−XTEN_AE864 Expression Vectors

The BDD FVIII fragment with NheI and SfiI flanking the 5′ and 3′ end is generated by digesting the pBC0025 construct. This digested fragment is then ligated to a NheI/SfiI digested pSecTag vector (pBC0048 pSecTag-FVIII−/−XTEN_AE864) encoding the FVIII followed by the XTEN_AE864 sequence. The ligated DNA mixture is used to transform DH5a bacterial cells. Transformants are screened by DNA miniprep and the desired constructs are confirmed by DNA sequencing. The final construct is pBC0060, which encodes the BDD FVIII−/−XTEN_AE864 protein under the control of a human CMV promoter. Introduction of the pBC0060 construct into mammalian cells is expected to express the FVIII protein with a C terminal XTEN fusion (BDD FVIII−/−XTEN_AE864) with procoagulant activity.

Construction of BDD FVIII-/FXI/-XTEN_AE864 Expression Vectors

The BDD FVIII fragment with NheI and SfiI flanking the 5′ and 3′ end is generated by digesting the pBC0025 construct. This digested fragment is then ligated to a NheI/SfiI digested pSecTag vector (pBC0047 pSecTag-FVIII-/FXI/-XTEN_AE864) encoding the FVIII followed by the FXI cleavage sequence (/FXI/) and XTEN_AE864. The ligated DNA mixture is used to transform DH5a bacterial cells. Transformants are screened by DNA miniprep and the desired constructs are confirmed by DNA sequencing. The final construct is pBC0051, which encodes the BDD FVIII-/FXI/-XTEN_AE864 protein under the control of a human CMV promoter. Introduction of the pBC0051 construct into mammalian cells is expected to express the FVIII protein with a C terminal XTEN fusion (BDD FVIII-/FXI/-XTEN_AE864), which could be subsequently cleaved by FXI, therefore liberating the BDD FVIII protein with procoagulant activity.

Construction of BDD FVIII−/−XTEN Expression Vectors Comprising AE288 or AG288

The fused AE864 XTEN sequence in pBC0060 is replaced by digesting the XTEN sequences AE288 and AG288 with BsaI and HindIII. A subsequent ligation step using the respective AE288 or AG288 XTEN fragment and BsaI/HindIII digested pBC0051 allows the exchange of the AE288 or AG288 sequences into the BDD FVIII expression vector. The resulting final constructs are pBC0061 for BDD FVIII-AE288 and pBC0062 for BDD FVIII-AG288. Introduction of the pBC0061 construct into mammalian cells is expected to express the FVIII protein with a C-terminal AE288 XTEN fusion (BDD FVIII−/−XTEN_AE288) with procoagulant activity. Introduction of the pBC0062 construct into mammalian cells is expected to express the FVIII protein with a C-terminal AG288 XTEN fusion (BDD FVIII−/−XTEN_AG288) with procoagulant activity.

Construction of BDD FVIII-/FXI/-XTEN Expression Vectors with Alternate XTEN

The fused XTEN sequence in pBC0051 is replaced by digesting DNA encoding other XTEN sequences (e.g. other variants and lengths of XTEN; e.g. AE42, AG42, AG288, AM288) with BsaI and HindIII. A ligation using the XTEN fragment and BsaI/HindIII digested pBC0051 allows the exchange of the various XTEN-encoding sequences into the BDD FVIII expression vector, providing the alternate constructs. Introduction of the alternate constructs into mammalian cells is expected to express the FVIII protein with a C-terminal XTEN (BDD FVIII-/FXI/-XTEN) that can be subsequently cleaved by FXI, releasing the FVIII, resulting in procoagulant FVIII fusion with procoagulant activity.

### Example 18

### Construction of Expression Plasmids for FVIII Signal Peptide-XTEN-/FXI/-BDD FVIII

Construction of Expression Vectors for FVIII Signal Peptide-XTEN_AE864

The coding sequences for the FVIII signal peptide is generated by annealing the following two oligos: 5′-CTAGCATGCAAATAGAGCTCTCCACCTGCTTCTTTCTGTGCCTTTTGCGATTCTGCTTTAGTGG GTCTCC-3′; 5′-ACCTGGAGACCCACTAAAGCAGAATCGCAAAAGGCACAGAAAGAAGCAGGTGGAGAGCTCT ATTTGCATG-3′. The annealed oligos are flanked by the NheI and BsaI restriction enzyme sites on either end, and is ligated to NheI/BsaI digested pCWO645 vector which encodes the FVII-XTEN_AE864. The ligated DNA mixture is used to transform DH5a bacterial cells. Transformants is screened by DNA miniprep and the desired constructs are confirmed by DNA sequencing. The final construct is designated pBC0029, which encodes the signal peptide-XTEN_AE864 protein under the control of a human CMV promoter. This construct is used as an intermediate construct for creating an expression construct with XTEN fused on the N-terminus of the FVIII protein, and can also be used as a master plasmid for creating expression constructs that allow XTEN fusion on the N-terminus of a secreted protein.

Construction of Signal Peptide-XTEN_AE864-/FXI/-BDD FVIII Expression Vectors

An 1800 bp fragment within the FVIII coding region is amplified using primers that introduce NheI-BbsI-/FXI/-AgeI sites on the 5′ and endogenous KpnI restriction enzyme on the 3′ end. The NheI/KpnI digested FVIII fragment is ligated with NheI/KpnI digested pBC0027 vector. The ligated DNA mixture is used to transform DH5a bacterial cells. Transformants are screened by DNA miniprep and the desired constructs are confirmed by DNA sequencing. The resulting construct is designated pBC0052, which contains sequences that encode the /FXI/-FVIII protein without the FVIII signal peptide. This construct is used as an intermediate construct for creating an expression construct with XTEN fused on the N-terminus of the FVIII protein.

The pBC0052 vector is digested with BbsI/XhoI enzymes, and is used to ligate with Bbsi/XhoI digested pBC0029. The ligated DNA mixture is used to transform DH5a bacterial cells. Transformants are screened by DNA miniprep and the desired constructs are confirmed by DNA sequencing. The final construct is designated pBC0053, which encodes the signal peptide-XTEN_AE864-/FXI/-BDD FVIII protein under the control of a human CMV promoter. Introduction of the pBC0053 construct into mammalian cells is expected to express the FVIII protein with an N-terminal XTEN fusion (signal peptide-XTEN_AE864-/FXI/-BDD FVIII), which could be subsequently cleaved by FXI, therefore liberating the BDD FVIII protein.

Construction of Signal Peptide-XTEN-/FXI/-BDD FVIII Expression Vectors

The fused XTEN sequence in pBC0053 can be replaced by digesting other XTEN fragments (e.g. AM, AF, AG) with BsaI and BbsI. A ligation using the XTEN fragment and BsaI/BbsI digested pBC0053 allows the exchange of various XTEN pieces (e.g. AM, AF, AG) into the BDD FVIII expression vector. Various XTEN fusions can increase the half lives of these proteins differently, allowing modification of the properties (e.g. efficacy, potency) of these proteins. Introduction of any of these fusion constructs into mammalian cells is expected to express the FVIII protein with an N-terminal XTEN fusion (signal peptide-XTEN-/FXI/-BDD FVIII), in which the fused XTEN peptide can be subsequently cleaved by FXI, generating the BDD FVIII protein.

### Example 19

### Construction of BDD FVIII with Interdomain XTEN Insertion

Construction of BDD FVIII Expression Vectors with an XTEN Insertion at the A2-B Domain Boundaries

The pBC0027 construct (pMK-BDD FVIII-STOP) is a cloning vector designed to contain the BDD FVIII protein coding sequences, but not a promoter positioned to initiate the expression of BDD FVIII. This construct is used for manipulation of the coding sequences of BDD FVIII as the vector backbone contains very few restriction enzyme sites. therefore allowing easy cloning strategies. The BDD FVIII proteins contain 1457 amino acids at a total molecular weight of 167539.66. There are 6 domains within the wild-type FVIII protein, the A1, A2, B, A3, C1 and C2 domains. In the BDD FVIII protein, most of the B domain has been deleted as it is believed to be an unstructured domain and the removal of the domain does not alter critical functions of this protein. However, the B domain boundaries seem to be excellent positions for creating XTEN fusions to allow extension of the protein half lives.

Within the pBC0027 construct, there is a unique HindIII restriction enzyme site at the boundary of A2-B junction. The XTEN (e.g., sequences of Tables 4, or 8-12) are amplified using primers that introduce a HindIII and FXI cleavage site on either end of the XTEN coding sequence. The fused XTEN sequence can be altered by amplifying various XTEN fragments. Various XTEN fusions can increase the half lives of these proteins differently, allowing modification of the properties (e.g. efficacy, potency) of these proteins. The HindIII-/FXI/-XTEN-/FXV-HindIII fragment is digested with HindIII and ligated with HindIII digested pBC0027. The ligated DNA mixture is used to transform DH5a bacterial cells. Transformants are screened by DNA miniprep and the desired constructs are confirmed by DNA sequencing. The final construct is designated pBC0054, which encodes the BDD FVIII protein with an interdomain XTEN fusion (FVIII(A1-A2)-/FXI/-XTEN-/FXI/-FVIII(C1-C2)) but not a promoter to initiate gene expression.

The pBC0054 construct is digested with NheI/SalI, and ligated with NheI/SalI digested CET1019-HS vector (Millipore). The CET1019-HS vector contains a human CMV promoter and a UCOE sequence to facilitate gene expression. The ligated DNA mixture is used to transform DH5a bacterial cells. Transformants are screened by DNA miniprep and the desired constructs are confirmed by DNA sequencing. The final construct is designated pBC0055 (CET1019-HS-FVIII(A1-A2)-/FXI/-XTEN-/FXI/-FVIII(C1-C2)), which encodes the BDD FVIII protein with an interdomain (inter-A2/B domain) XTEN fusion (FVIII(A1-A2)-/FXI/-XTEN-/FXI/-FVIII(C1-C2)) under the control of a human CMV promoter. Introduction of the pBC0055 construct into mammalian cells is expected to express the BDD FVIII protein with an interdomain XTEN fusion (FVIII(A1-A2)-/FXI/-XTEN-/FXI/-FVIII(C1-C2)), which could be subsequently cleaved by FXI, therefore liberating the BDD FVIII protein.

Construction of BDD FVIII Expression Vectors with an XTEN Insertion at the A1-A2 Domain Boundaries

The pBC0027 construct is designed as a template for two PCR reactions using the following four primers:

(Reaction I)
5′-ATGATGGCATGGAAGCCTAT-3′; 5′-ATCCCTCACCTTCGCCAGA
ACCTTCAGAACCCTCACCTTCAGAACCTTCACCAGAACCTTCACCATCTT
CCGCTTCTTCATTATTTTTCAT-3′.
(Reaction II)
5′-TTCTGGCGAAGGTGAGGGATCTGAAGGCGGTTCTGAAGGTGAAGGTG
GCTCTGAGGGTTCCGAATATGATGATGATCTTACTGATTCTGAAAT-3′;
5′-TATTCTCTGTGAGGTACCAGC-3′.

The PCR products generated are 150 bps and 800 bps respectively. The 800 bp product is used as the template for the next round of PCR reaction with the 150 bp product as one primer and 5′-TATTCTCTGTGAGGTACCAGC-3′ as the other. The product for the second round of PCR is 930 bps and is digested with PshAI and ACC65I restriction enzymes. This PshAI/Acc65I flanked DNA fragment is ligated with PshAI/Acc65I digested pBC0027. The ligated DNA mixture is used to transform DH5a bacterial cells. Transformants is screened by DNA miniprep and the desired constructs are confirmed by DNA sequencing. The final construct is designated pBC0058 (pMK-BDD FVIII-D345-XTEN_Y36), which encodes the BDD FVIII protein with an interdomain (inter-A1/A2 domain) XTEN fusion after the D345 residue.

The pBC0058 construct is digested with NheI/SalI, and ligated with NheI/SalI digested CET1019-HS vector (Millipore). The CET1019-HS vector contains a human CMV promoter and a UCOE sequence to facilitate gene expression. The ligated DNA mixture is used to transform DH5a bacterial cells. Transformants are screened by DNA miniprep and the desired constructs are confirmed by DNA sequencing. The final construct is designated pBC0059 (CET1019-HS-BDD FVIII D345-XTEN_Y36), which encodes the BDD FVIII protein with an interdomain (inter-A1/A2 domain) XTEN fusion after the D345 residue under the control of a human CMV promoter. Introduction of the pBC0059 construct into mammalian cells is expected to express the BDD FVIII protein with an interdomain XTEN fusion (BDD FVIII D345-XTEN_Y36).

### Example 20

### Construction of FVIII with Intradomain XTEN Insertion

Construction of BDD FVIII Expression Vectors with an XTEN Insertion after P598 (within the A2 Domain)

The coding sequences for XTEN_Y36 is amplified using PCR techniques with the following primers: 5′-GAAGCTGGTACCTCACAGAGAATATACAACGCTTTCTCCCCAATCCAGGTGAAGGTTCTGGTG AAGG-3′ 5′-AACTCTGGATCCTCAAGCTGCACTCCAGCTTCGGAACCCTCAGAGCC-3′.

The 184 bp PCR product is flanked by the KpnI and BamHI restriction enzyme sites on either end, and is ligated to KpnII/BamHI digested pBC0027 vector which encodes the BDD FVIII gene. The ligated DNA mixture is used to transform DH5a bacterial cells. Transformants are screened by DNA miniprep and the desired constructs are confirmed by DNA sequencing. The final construct is designated pBC0056, which contains DNA sequences encoding the FVIII protein with an XTEN_Y36 fusion after the P598 residue. This cloning strategy is used to introduce various forms of XTEN into the BDD FVIII protein by altering the template for the PCR reaction and changing the primers accordingly.

The pBC0056 construct is digested with NheI/SalI, and ligated with NheI/SalI digested CET1019-HS vector (Millipore). The CET1019-HS vector contains a human CMV promoter and a UCOE sequence to facilitate gene expression. The ligated DNA mixture is used to transform DH5a bacterial cells. Transformants are screened by DNA miniprep and the desired constructs are confirmed by DNA sequencing. The final construct is designated pBC0057 (CET1019-HS-FVIII P598-XTEN_Y32), which encodes the BDD FVIII protein with an intradomain (within A2 domain) XTEN fusion under the control of a human CMV promoter. Introduction of the pBC0057 construct into mammalian cells is expected to express the BDD FVIII protein with an intradomain XTEN fusion (FVIII P598-XTEN_Y32).

Construction of BDD FVIII Expression Vectors with Other Intradomain XTEN Insertions

To introduce various XTEN segments into other intradomain sites within BDD FVIII (e.g., the XTEN of Tables 4, or 8-12), primers are designed that amplify XTEN with an overhang that can anneal with BDD FVIII. The coding sequence of FVIII (pMK-BDD FVIII) is designed with various unique restriction enzyme sites to allow these specific insertions. The unique restriction enzymes are listed below with their cut site: NheI 376, SacI 391, AfiII 700, SpeI 966, PshAI 1417, Acc65I 2192, KpnI 2192, BamHI 2250, HindIII 2658, PfoI 2960, PflMI 3413, ApaI 3893, Bsp1201 3893, SwaI 4265, OliI 4626, XbaI 4644, BstBI 4673, SalI 4756, and XhoI 4762. The NheI and SalI sites on either end of the coding sequence are used to insert the DNA fragment into a human CMV promoter driven vector, the CET1019-HS (Millipore) for expression in mammalian cells. These constructs are expected to express the BDD FVIII protein with an XTEN fusion.

TABLE 14
DNA and Amino Acid Sequences of FVIII-XTEN Constructs

| Construct | Amino acid | |
|---|---|---|
| Name | sequence | Nucleotide sequence |
| pBC0100 | MAYTDETFKTREAIQ | ATGGCATACACAGATGAAACCTTTAAGACTCGTGAAGCTATTC |
| | HESGILGPLLYGEVG | AGCATGAATCAGGAATCTTGGGACCTTTACTTTATGGGGAAGT |
| | DTLLIIFKNQASRPYN | TGGAGACACACTGTTGATTATATTTAAGAATCAAGCAAGCAGA |
| | IYPHGITDVRPLYSRR | CCATATAACATCTACCCTCACGGAATCACTGATGTCCGTCCTTT |
| | LPKGVKHLKDFPILPG | GTATTCAAGGAGATTACCAAAAGGTGTAAAACATTTGAAGGA |
| | EIFKYKWTVTVEDGP | TTTTCCAATTCTGCCAGGAGAAATATTCAAATATAAATGGACA |
| | TKSDPRCLTRYYSSF | GTGACTGTAGAAGATGGGCCAACTAAATCAGATCCTCGGTGCC |
| | VNMERDLASGLIGPL | TGACCCGCTATTACTCTAGTTTCGTTAATATGGAGAGAGATCT |
| | LICYKESVDQRGNQI | AGCTTCAGGACTCATTGGCCCTCTCCTCATCTGCTACAAAGAA |
| | MSDKRNVILFSVFDE | TCTGTAGATCAAAGAGGAAACCAGATAATGTCAGACAAGAGG |
| | NRSWYLTENIQRFLP | AATGTCATCCTGTTTTCTGTATTTGATGAGAACCGAAGCTGGT |
| | NPAGVQLEDPEFQAS | ACCTCACAGAGAATATACAACGCTTTCTCCCCAATCCAGCTGG |
| | NIMHSINGYVFDSLQ | AGTGCAGCTTGAGGATCCAGAGTTCCAAGCCTCCAACATCATG |
| | LSVCLHEVAYWYILSI | CACAGCATCAATGGCTATGTTTTTGATAGTTTGCAGTTGTCAGT |
| | GAQTDFLSVFFSGYT | TTGTTTGCATGAGGTGGCATACTGGTACATTCTAAGCATTGGA |
| | FKHKMVYEDTLTLFP | GCACAGACTGACTTCCTTTCTGTCTTCTTCTCTGGATATACCTT |
| | FSGETVFMSMENPGL | CAAACACAAAATGGTCTATGAAGACACACTCACCCTATTCCCA |
| | WILGCHNSDFRNRG | TTCTCAGGAGAAACTGTCTTCATGTCGATGGAAAACCCAGGTC |
| | MTALLKVSSCDKNTG | TATGGATTCTGGGGTGCCACAACTCAGACTTTCGGAACAGAGG |
| | DYYEDSYEDISAYLL | CATGACCGCCTTACTGAAGGTTTCTAGTTGTGACAAGAACACT |
| | SKNNAIEPRSFSQNPP | GGTGATTATTACGAGGACAGTTATGAAGATATTTCAGCATACT |
| | VLKRHQREITRTTLQS | TGCTGAGTAAAAACAATGCCATTGAACCAAGAAGCTTCTCTCA |
| | DQEEIDYDDTISVEM | AAACCCACCAGTCTTGAAACGCCATCAACGGGAAATAACTCG |
| | KKEDFDIYDEDENQS | TACTACTCTTCAGTCAGATCAAGAGGAAATTGACTATGATGAT |
| | PRSFQKKTRHYFIAA | ACCATATCAGTTGAAATGAAGAAGGAAGATTTTGACATTTATG |
| | VERLWDYGMSSSPH | ATGAGGATGAAAATCAGAGCCCCCGCAGCTTTCAAAAGAAAA |
| | VLRNRAQSGSVPQFK | CACGACACTATTTTATTGCTGCAGTGGAGAGGCTCTGGGATTA |
| | KVVFQEFTDGSFTQP | TGGGATGAGTAGCTCCCCACATGTTCTAAGAAACAGGGCTCAG |
| | LYRGELNEHLGLLGP | AGTGGCAGTGTCCCTCAGTTCAAGAAAGTTGTTTTCCAGGAAT |
| | YIRAEVEDNIMVTFR | TTACTGATGGCTCCTTTACTCAGCCCTTATACCGTGGAGAACT |
| | NQASRPYSFYSSLISY | AAATGAACATTTGGGACTCCTGGGGCCATATATAAGAGCAGA |
| | EEDQRQGAEPRKNFV | AGTTGAAGATAATATCATGGTAACTTTCAGAAATCAGGCCTCT |
| | KPNETKTYFWKVQH | CGTCCCTATTCCTTCTATTCTAGCCTTATTTCTTATGAGGAAGA |
| | HMAPTKDEFDCKAW | TCAGAGGCAAGGAGCAGAACCTAGAAAAAACTTTGTCAAGCC |
| | AYFSDVDLEKDVHSG | TAATGAAACCAAAACTTACTTTTGGAAAGTGCAACATCATATG |
| | LIGPLLVCHTNTLNPA | GCACCCACTAAAGATGAGTTTGACTGCAAAGCCTGGGCTTATT |
| | HGRQVTVQEFALFFTI | TCTCTGATGTTGACCTGGAAAAAGATGTGCACTCAGGCCTGAT |
| | FDETKSWYFTENMER | TGGACCCCTTCTGGTCTGCCACACTAACACACTGAACCCTGCT |
| | NCRAPCNIQMEDPTF | CATGGGAGACAAGTGACAGTACAGGAATTTGCTCTGTTTTTCA |
| | KENYRFHAINGYIMD | CCATCTTTGATGAGACCAAAAGCTGGTACTTCACTGAAAATAT |
| | TLPGLVMAQDQRIR | GGAAAGAAACTGCAGGGCTCCCTGCAATATCCAGATGGAAGA |
| | WYLLSMGSNENIHSI | TCCCACTTTTAAAGAGAATTATCGCTTCCATGCAATCAATGGC |
| | HFSGHVFTVRKKEEY | TACATAATGGATACACTACCTGGCTTAGTAATGGCTCAGGATC |
| | KMALYNLYPGVFET | AAAGGATTCGATGGTATCTGCTCAGCATGGGCAGCAATGAAA |
| | VEMLPSKAGIWRVEC | ACATCCATTCTATTCATTTCAGTGGACATGTGTTCACTGTACGA |
| | LIGEHLHAGMSTLFL | AAAAAAGAGGAGTATAAAATGGCACTGTACAATCTCTATCCA |
| | VYSNKCQTPLGMAS | GGTGTTTTTGAGACAGTGGAAATGTTACCATCCAAAGCTGGAA |
| | GHIRDFQITASGQYG | TTTGGCGGGTGGAATGCCTTATTGGCGAGCATCTACATGCTGG |
| | QWAPKLARLHYSGSI | GATGAGCACACTTTTTCTGGTGTACAGCAATAAGTGTCAGACT |
| | NAWSTKEPFSWIKVD | CCCCTGGGAATGGCTTCTGGACACATTAGAGATTTTCAGATTA |
| | LLAPMIIHGIKTQGAR | CAGCTTCAGGACAATATGGACAGTGGGCCCCAAAGCTGGCCA |
| | QKFSSLYISQFIIMYSL | GACTTCATTATTCCGGATCAATCAATGCCTGGAGCACCAAGGA |
| | DGKKWQTYRGNSTG | GCCCTTTTCTTGGATCAAGGTGGATCTGTTGGCACCAATGATT |
| | TLMVFFGNVDSSGIK | ATTCACGGCATCAAGACCCAGGGTGCCCGTCAGAAGTTCTCCA |
| | HNIFNPPIIARYIRLHP | GCCTCTACATCTCTCAGTTTATCATCATGTATAGTCTTGATGGG |
| | THYSIRSTLRMELMG | AAGAAGTGGCAGACTTATCGAGGAAATTCCACTGGAACCTTA |
| | CDLNSCSMPLGMESK | ATGGTCTTCTTTGGCAATGTGGATTCATCTGGGATAAAACACA |
| | AISDAQITASSYFTNM | ATATTTTTAACCCTCCAATTATTGCTCGATACATCCGTTTGCAC |
| | FATWSPSKARLHLQG | CCAACTCATTATAGCATTCGCAGCACTCTTCGCATGGAGTTGA |
| | RSNAWRPQVNNPKE | TGGGCTGTGATTTAAATAGTTGCAGCATGCCATTGGGAATGGA |
| | WLQVDFQKTMKVTG | GAGTAAAGCAATATCAGATGCACAGATTACTGCTTCATCCTAC |
| | VTTQGVKSLLTSMYV | TTTACCAATATGTTTGCCACCTGGTCTCCTTCAAAAGCTCGACT |
| | KEFLISSSQDGHQWT | TCACCTCCAAGGGAGGAGTAATGCCTGGAGACCTCAGGTGAA |
| | LFFQNGKVKVFQGN | TAATCCAAAAGAGTGGCTGCAAGTGGACTTCCAGAAGACAAT |
| | QDSFTPVVNSLDPPLL | GAAAGTCACAGGAGTAACTACTCAGGGAGTAAAATCTCTGCTT |
| | TRYLRIHPQSWVHQI | ACCAGCATGTATGTGAAGGAGTTCCTCATCTCCAGCAGTCAAG |
| | ALRMEVLGCEAQDLY | ATGGCCATCAGTGGACTCTCTTTTTTCAGAATGGCAAAGTAAA |
| | | GGTTTTTCAGGGAAATCAAGACTCCTTCACACCTGTGGTGAAC |
| | | TCTCTAGACCCACCGTTACTGACTCGCTACCTTCGAATTCACCC |
| | | CCAGAGTTGGGTGCACCAGATTGCCCTGAGGATGGAGGTTCTG |
| | | GGCTGCGAGGCACAGGACCTCTACTGA |
| pBC0114 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKWQTYRGNSTG | GCTGGCCAGACTTCATTATTCCGGATCAATCAATGCCTGGAGC |
| | TLMVFFGNVDSSGIK | ACCAAGGAGCCCTTTTCTTGGATCAAGGTGGATCTGTTGGCAC |
| | HNIFNPPIIARYIRLHP | CAATGATTATTCACGGCATCAAGACCCAGGGTGCCCGTCAGAA |
| | THYSIRSTLRMELMG | GTTCTCCAGCCTCTACATCTCTCAGTTTATCATCATGTATAGTC |
| | CDLNSCSMPLGMESK | TTGATGGGAAGAAGTGGCAGACTTATCGAGGAAATTCCACTG |
| | AISDAQITASSYFTNM | GAACCTTAATGGTCTTCTTTGGCAATGTGGATTCATCTGGGAT |
| | FATWSPSKARLHLQG | AAAACACAATATTTTTAACCCTCCAATTATTGCTCGATACATC |
| | RSNAWRPQVNNPKE | CGTTTGCACCCAACTCATTATAGCATTCGCAGCACTCTTCGCAT |
| | WLQVDFQKTMKVTG | GGAGTTGATGGGCTGTGATTTAAATAGTTGCAGCATGCCATTG |
| | VTTQGVKSLLTSMYV | GGAATGGAGAGTAAAGCAATATCAGATGCACAGATTACTGCT |
| | KEFLISSSQDGHQWT | TCATCCTACTTTACCAATATGTTTGCCACCTGGTCTCCTTCAAA |
| | LFFQNGKVKVFQGN | AGCTCGACTTCACCTCCAAGGGAGGAGTAATGCCTGGAGACCT |
| | QDSFTPVVNSLDPPLL | CAGGTGAATAATCCAAAAGAGTGGCTGCAAGTGGACTTCCAG |
| | TRYLRIHPQSWVHQI | AAGACAATGAAAGTCACAGGAGTAACTACTCAGGGAGTAAAA |
| | ALRMEVLGCEAQDL | TCTCTGCTTACCAGCATGTATGTGAAGGAGTTCCTCATCTCCA |
| | YGAGSPGAETAEQKL | GCAGTCAAGATGGCCATCAGTGGACTCTCTTTTTTCAGAATGG |
| | ISEEDLSPATG | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0126 | MQIELSTCFFLCLLRF | ATGCAAATAGAGCTCTCCACCTGCTTCTTTCTGTGCCTTTTGCG |
| | CFSATRGAPGSPAGS | ATTCTGCTTTAGTGCCACCAGAGGCGCGCCAGGTTCTCCTGCT |
| | PTSTEEGTSESATPES | GGCTCCCCCACCTCAACAGAAGAGGGGACAAGCGAAAGCGCT |
| | GPGSEPATSGSETPAS | ACGCCTGAGAGTGGCCCTGGCTCTGAGCCAGCCACCTCCGGCT |
| | SRYYLGAVELSWDY | CTGAAACCCCTGCCTCGAGCAGATACTACCTGGGTGCAGTGGA |
| | MQSDLGELPVDARFP | ACTGTCATGGGACTATATGCAAAGTGATCTCGGTGAGCTGCCT |
| | PRVPKSFPFNTSVVY | GTGGACGCAAGATTTCCTCCTAGAGTGCCAAAATCTTTTCCAT |
| | KKTLFVEFTDHLFNIA | TCAACACCTCAGTCGTGTACAAAAAGACTCTGTTTGTAGAATT |
| | KPRPPWMGLLGPTIQ | CACGGATCACCTTTTCAACATCGCTAAGCCAAGGCCACCCTGG |
| | AEVYDTVVITLKNMA | ATGGGTCTGCTAGGTCCTACCATCCAGGCTGAGGTTTATGATA |
| | SHPVSLHAVGVSYW | CAGTGGTCATTACACTTAAGAACATGGCTTCCCATCCTGTCAG |
| | KASEGAEYDDQTSQR | TCTTCATGCTGTTGGTGTATCCTACTGGAAAGCTTCTGAGGGA |
| | EKEDDKVFPGGSHTY | GCTGAATATGATGATCAGACCAGTCAAAGGGAGAAAGAAGAT |
| | VWQVLKENGPMASD | GATAAAGTCTTCCCTGGTGGAAGCCATACATATGTCTGGCAGG |
| | PLCLTYSYLSHVDLV | TCCTGAAAGAGAATGGTCCAATGGCCTCTGACCCACTGTGCCT |
| | KDLNSGLIGALLVCR | TACCTACTCATATCTTTCTCATGTGGACCTGGTAAAAGACTTG |
| | EGSLAKEKTQTLHKFI | AATTCAGGCCTCATTGGAGCCCTACTAGTATGTAGAGAAGGGA |
| | LLFAVFDEGKSWHSE | GTCTGGCCAAGGAAAAGACACAGACCTTGCACAAATTTATACT |
| | TKNSLMQDRDAASA | ACTTTTTGCTGTATTTGATGAAGGGAAAAGTTGGCACTCAGAA |
| | RAWPKMHTVNGYVN | ACAAAGAACTCCTTGATGCAGGATAGGGATGCTGCATCTGCTC |
| | RSLPGLIGCHRKSVY | GGGCCTGGCCTAAAATGCACACAGTCAATGGTTATGTAAACA |
| | WHVIGMGTTPEVHSI | GGTCTCTGCCAGGTCTGATTGGATGCCACAGGAAATCAGTCTA |
| | FLEGHTFLVRNHRQA | TTGGCATGTGATTGGAATGGGCACCACTCCTGAAGTGCACTCA |
| | SLEISPITFLTAQTLLM | ATATTCCTCGAAGGTCACACATTTCTTGTGAGGAACCATCGCC |
| | DLGQFLLFCHISSHQH | AGGCTAGCTTGGAAATCTCGCCAATAACTTTCCTTACTGCTCA |
| | DGMEAYVKVDSCPE | AACACTCTTGATGGACCTTGGACAGTTTCTACTGTTTTGTCATA |
| | EPQLRMKNNEEAED | TCTCTTCCCACCAACATGATGGCATGGAAGCTTATGTCAAAGT |
| | YDDDLTDSEMDVVR | AGACAGCTGTCCAGAGGAACCCCAACTACGAATGAAAAATAA |
| | FDDDNSPSFIQIRSVA | TGAAGAAGCGGAAGACTATGATGATGATCTTACTGATTCTGAA |
| | KKHPKTWVHYIAAEE | ATGGATGTGGTCAGGTTTGATGATGACAACTCTCCTTCCTTTAT |
| | EDWDYAPLVLAPDD | CCAAATTCGCTCAGTTGCCAAGAAGCATCCTAAAACTTGGGTA |
| | RSYKSQYLNNGPQRI | CATTACATTGCTGCTGAAGAGGAGGACTGGGACTATGCTCCCT |
| | GRKYKKVRFMAYTD | TAGTCCTCGCCCCCGATGACAGAAGTTATAAAAGTCAATATTT |
| | ETFKTREAIQHESGIL | GAACAATGGCCCTCAGCGGATTGGTAGGAAGTACAAAAAAGT |
| | GPLLYGEVGDTLLIIF | CCGATTTATGGCATACACAGATGAAACCTTTAAGACTCGTGAA |
| | KNQASRPYNIYPHGIT | GCTATTCAGCATGAATCAGGAATCTTGGGACCTTTACTTTATG |
| | DVRPLYSRRLPKGVK | GGGAAGTTGGAGACACACTGTTGATTATATTTAAGAATCAAGC |
| | HLKDFPILPGEIFKYK | AAGCAGACCATATAACATCTACCCTCACGGAATCACTGATGTC |
| | WTVTVEDGPTKSDPR | CGTCCTTTGTATTCAAGGAGATTACCAAAAGGTGTAAAACATT |
| | CLTRYYSSFVNMERD | TGAAGGATTTTCCAATTCTGCCAGGAGAAATATTCAAATATAA |
| | LASGLIGPLLICYKES | ATGGACAGTGACTGTAGAAGATGGGCCAACTAAATCAGATCC |
| | VDQRGNQIMSDKRN | TCGGTGCCTGACCCGCTATTACTCTAGTTTCGTTAATATGGAG |
| | VILFSVFDENRSWYL | AGAGATCTAGCTTCAGGACTCATTGGCCCTCTCCTCATCTGCT |
| | TENIQRFLPNPAGVQL | ACAAAGAATCTGTAGATCAAAGAGGAAACCAGATAATGTCAG |
| | EDPEFQASNIMHSING | ACAAGAGGAATGTCATCCTGTTTTCTGTATTTGATGAGAACCG |
| | YVFDSLQLSVCLHEV | AAGCTGGTACCTCACAGAGAATATACAACGCTTTCTCCCCAAT |
| | AYWYILSIGAQTDFLS | CCAGCTGGAGTGCAGCTTGAGGATCCAGAGTTCCAAGCCTCCA |
| | VFFSGYTFKHKMVYE | ACATCATGCACAGCATCAATGGCTATGTTTTTGATAGTTTGCA |
| | DTLTLFPFSGETVFMS | GTTGTCAGTTTGTTTGCATGAGGTGGCATACTGGTACATTCTA |
| | MENPGLWILGCHNSD | AGCATTGGAGCACAGACTGACTTCCTTTCTGTCTTCTTCTCTGG |
| | FRNRGMTALLKVSSC | ATATACCTTCAAACACAAAATGGTCTATGAAGACACACTCACC |
| | DKNTGDYYEDSYEDI | CTATTCCCATTCTCAGGAGAAACTGTCTTCATGTCGATGGAAA |
| | SAYLLSKNNAIEPRSF | ACCCAGGTCTATGGATTCTGGGGTGCCACAACTCAGACTTTCG |
| | SQNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0127 | MQIELSTCFFLCLLRF | ATGCAAATAGAGCTCTCCACCTGCTTCTTTCTGTGCCTTTTGCG |
| | CFSATRGAPGSPAGS | ATTCTGCTTTAGTGCCACCAGAGGCGCGCCAGGTTCTCCTGCT |
| | PTSTEEGTSESATPES | GGCTCCCCCACCTCAACAGAAGAGGGGACAAGCGAAAGCGCT |
| | GPGSEPATSGSETPAS | ACGCCTGAGAGTGGCCCTGGCTCTGAGCCAGCCACCTCCGGCT |
| | SATRRYYLGAVELSW | CTGAAACCCCTGCCTCGAGCGCTACAAGAAGATACTACCTGGG |
| | DYMQSDLGELPVDA | TGCAGTGGAACTGTCATGGGACTATATGCAAAGTGATCTCGGT |
| | RFPPRVPKSFPFNTSV | GAGCTGCCTGTGGACGCAAGATTTCCTCCTAGAGTGCCAAAAT |
| | VYKKTLFVEFTDHLF | CTTTTCCATTCAACACCTCAGTCGTGTACAAAAAGACTCTGTTT |
| | NIAKPRPPWMGLLGP | GTAGAATTCACGGATCACCTTTTCAACATCGCTAAGCCAAGGC |
| | TIQAEVYDTVVITLK | CACCCTGGATGGGTCTGCTAGGTCCTACCATCCAGGCTGAGGT |
| | NMASHPVSLHAVGV | TTATGATACAGTGGTCATTACACTTAAGAACATGGCTTCCCAT |
| | SYWKASEGAEYDDQ | CCTGTCAGTCTTCATGCTGTTGGTGTATCCTACTGGAAAGCTTC |
| | TSQREKEDDKVFPGG | TGAGGGAGCTGAATATGATGATCAGACCAGTCAAAGGGAGAA |
| | SHTYVWQVLKENGP | AGAAGATGATAAAGTCTTCCCTGGTGGAAGCCATACATATGTC |
| | MASDPLCLTYSYLSH | TGGCAGGTCCTGAAAGAGAATGGTCCAATGGCCTCTGACCCAC |
| | VDLVKDLNSGLIGAL | TGTGCCTTACCTACTCATATCTTTCTCATGTGGACCTGGTAAAA |
| | LVCREGSLAKEKTQT | GACTTGAATTCAGGCCTCATTGGAGCCCTACTAGTATGTAGAG |
| | LHKFILLFAVFDEGKS | AAGGGAGTCTGGCCAAGGAAAAGACACAGACCTTGCACAAAT |
| | WHSETKNSLMQDRD | TTATACTACTTTTTGCTGTATTTGATGAAGGGAAAAGTTGGCA |
| | AASARAWPKMHTVN | CTCAGAAACAAAGAACTCCTTGATGCAGGATAGGGATGCTGC |
| | GYVNRSLPGLIGCHR | ATCTGCTCGGGCCTGGCCTAAAATGCACACAGTCAATGGTTAT |
| | KSVYWHVIGMGTTPE | GTAAACAGGTCTCTGCCAGGTCTGATTGGATGCCACAGGAAAT |
| | VHSIFLEGHTFLVRNH | CAGTCTATTGGCATGTGATTGGAATGGGCACCACTCCTGAAGT |
| | RQASLEISPITFLTAQT | GCACTCAATATTCCTCGAAGGTCACACATTTCTTGTGAGGAAC |
| | LLMDLGQFLLFCHISS | CATCGCCAGGCTAGCTTGGAAATCTCGCCAATAACTTTCCTTA |
| | HQHDGMEAYVKVDS | CTGCTCAAACACTCTTGATGGACCTTGGACAGTTTCTACTGTTT |
| | CPEEPQLRMKNNEEA | TGTCATATCTCTTCCCACCAACATGATGGCATGGAAGCTTATG |
| | EDYDDDLTDSEMDV | TCAAAGTAGACAGCTGTCCAGAGGAACCCCAACTACGAATGA |
| | VRFDDDNSPSFIQIRS | AAAATAATGAAGAAGCGGAAGACTATGATGATGATCTTACTG |
| | VAKKHPKTWVHYIA | ATTCTGAAATGGATGTGGTCAGGTTTGATGATGACAACTCTCC |
| | AEEEDWDYAPLVLAP | TTCCTTTATCCAAATTCGCTCAGTTGCCAAGAAGCATCCTAAA |
| | DDRSYKSQYLNNGPQ | ACTTGGGTACATTACATTGCTGCTGAAGAGGAGGACTGGGACT |
| | RIGRKYKKVRFMAYT | ATGCTCCCTTAGTCCTCGCCCCCGATGACAGAAGTTATAAAAG |
| | DETFKTREAIQHESGI | TCAATATTTGAACAATGGCCCTCAGCGGATTGGTAGGAAGTAC |
| | LGPLLYGEVGDTLLII | AAAAAAGTCCGATTTATGGCATACACAGATGAAACCTTTAAG |
| | FKNQASRPYNIYPHGI | ACTCGTGAAGCTATTCAGCATGAATCAGGAATCTTGGGACCTT |
| | TDVRPLYSRRLPKGV | TACTTTATGGGGAAGTTGGAGACACACTGTTGATTATATTTAA |
| | KHLKDFPILPGEIFKY | GAATCAAGCAAGCAGACCATATAACATCTACCCTCACGGAAT |
| | KWTVTVEDGPTKSDP | CACTGATGTCCGTCCTTTGTATTCAAGGAGATTACCAAAAGGT |
| | RCLTRYYSSFVNMER | GTAAAACATTTGAAGGATTTTCCAATTCTGCCAGGAGAAATAT |
| | DLASGLIGPLLICYKE | TCAAATATAAATGGACAGTGACTGTAGAAGATGGGCCAACTA |
| | SVDQRGNQIMSDKRN | AATCAGATCCTCGGTGCCTGACCCGCTATTACTCTAGTTTCGTT |
| | VILFSVFDENRSWYL | AATATGGAGAGAGATCTAGCTTCAGGACTCATTGGCCCTCTCC |
| | TENIQRFLPNPAGVQL | TCATCTGCTACAAAGAATCTGTAGATCAAAGAGGAAACCAGA |
| | EDPEFQASNIMHSING | TAATGTCAGACAAGAGGAATGTCATCCTGTTTTCTGTATTTGA |
| | YVFDSLQLSVCLHEV | TGAGAACCGAAGCTGGTACCTCACAGAGAATATACAACGCTTT |
| | AYWYILSIGAQTDFLS | CTCCCCAATCCAGCTGGAGTGCAGCTTGAGGATCCAGAGTTCC |
| | VFFSGYTFKHKMVYE | AAGCCTCCAACATCATGCACAGCATCAATGGCTATGTTTTTGA |
| | DTLTLFPFSGETVFMS | TAGTTTGCAGTTGTCAGTTTGTTTGCATGAGGTGGCATACTGGT |
| | MENPGLWILGCHNSD | ACATTCTAAGCATTGGAGCACAGACTGACTTCCTTTCTGTCTTC |
| | FRNRGMTALLKVSSC | TTCTCTGGATATACCTTCAAACACAAAATGGTCTATGAAGACA |
| | DKNTGDYYEDSYEDI | CACTCACCCTATTCCCATTCTCAGGAGAAACTGTCTTCATGTCG |
| | SAYLLSKNNAIEPRSF | ATGGAAAACCCAGGTCTATGGATTCTGGGGTGCCACAACTCAG |
| | SQNPPVLKRHQREITR | ACTTTCGGAACAGAGGCATGACCGCCTTACTGAAGGTTTCTAG |
| | TTLQSDQEEIDYDDTI | TTGTGACAAGAACACTGGTGATTATTACGAGGACAGTTATGAA |
| | SVEMKKEDFDIYDED | GATATTTCAGCATACTTGCTGAGTAAAAACAATGCCATTGAAC |
| | ENQSPRSFQKKTRHY | CAAGAAGCTTCTCTCAAAACCCACCAGTCTTGAAACGCCATCA |
| | FIAAVERLWDYGMSS | ACGGGAAATAACTCGTACTACTCTTCAGTCAGATCAAGAGGA |
| | SPHVLRNRAQSGSVP | AATCGATTATGATGATACCATATCAGTTGAAATGAAGAAGGA |
| | QFKKVVFQEFTDGSF | AGATTTTGACATTTATGATGAGGATGAAAATCAGAGCCCCCGC |
| | TQPLYRGELNEHLGL | AGCTTTCAAAAGAAAACACGACACTATTTTATTGCTGCAGTGG |
| | LGPYIRAEVEDNIMV | AGAGGCTCTGGGATTATGGGATGAGTAGCTCCCCACATGTTCT |
| | TFRNQASRPYSFYSSL | AAGAAACAGGGCTCAGAGTGGCAGTGTCCCTCAGTTCAAGAA |
| | ISYEEDQRQGAEPRK | AGTTGTTTTCCAGGAATTTACTGATGGCTCCTTTACTCAGCCCT |
| | NFVKPNETKTYFWK | TATACCGTGGAGAACTAAATGAACATTTGGGACTCCTGGGGCC |
| | VQHHMAPTKDEFDC | ATATATAAGAGCAGAAGTTGAAGATAATATCATGGTAACTTTC |
| | KAWAYFSDVDLEKD | AGAAATCAGGCCTCTCGTCCCTATTCCTTCTATTCTAGCCTTAT |
| | VHSGLIGPLLVCHTN | TTCTTATGAGGAAGATCAGAGGCAAGGAGCAGAACCTAGAAA |
| | TLNPAHGRQVTVQEF | AAACTTTGTCAAGCCTAATGAAACCAAAACTTACTTTTGGAAA |
| | ALFFTIFDETKSWYFT | GTGCAACATCATATGGCACCCACTAAAGATGAGTTTGACTGCA |
| | ENMERNCRAPCNIQM | AAGCCTGGGCTTATTTCTCTGATGTTGACCTGGAAAAAGATGT |
| | EDPTFKENYRFHAIN | GCACTCAGGCCTGATTGGACCCCTTCTGGTCTGCCACACTAAC |
| | GYIMDTLPGLVMAQ | ACACTGAACCCTGCTCATGGGAGACAAGTGACAGTACAGGAA |
| | DQRIRWYLLSMGSNE | TTTGCTCTGTTTTTCACCATCTTTGATGAGACCAAAAGCTGGTA |
| | NIHSIHFSGHVFTVRK | CTTCACTGAAAATATGGAAAGAAACTGCAGGGCTCCCTGCAAT |
| | KEEYKMALYNLYPG | ATCCAGATGGAAGATCCCACTTTTAAAGAGAATTATCGCTTCC |
| | VFETVEMLPSKAGIW | ATGCAATCAATGGCTACATAATGGATACACTACCTGGCTTAGT |
| | RVECLIGEHLHAGMS | AATGGCTCAGGATCAAAGGATTCGATGGTATCTGCTCAGCATG |
| | TLFLVYSNKCQTPLG | GGCAGCAATGAAAACATCCATTCTATTCATTTCAGTGGACATG |
| | MASGHIRDFQITASG | TGTTCACTGTACGAAAAAAAGAGGAGTATAAAATGGCACTGT |
| | QYGQWAPKLARLHY | ACAATCTCTATCCAGGTGTTTTTGAGACAGTGGAAATGTTACC |
| | SGSINAWSTKEPFSWI | ATCCAAAGCTGGAATTTGGCGGGTGGAATGCCTTATTGGCGAG |
| | KVDLLAPMIIHGIKTQ | CATCTACATGCTGGGATGAGCACACTTTTTCTGGTGTACAGCA |
| | GARQKFSSLYISQFII | ATAAGTGTCAGACTCCCCTGGGAATGGCTTCTGGACACATTAG |
| | MYSLDGKKWQTYRG | AGATTTTCAGATTACAGCTTCAGGACAATATGGACAGTGGGCC |
| | NSTGTLMVFFGNVDS | CCAAAGCTGGCCAGACTTCATTATTCCGGATCAATCAATGCCT |
| | SGIKHNIFNPPIIARYI | GGAGCACCAAGGAGCCCTTTTCTTGGATCAAGGTGGATCTGTT |
| | RLHPTHYSIRSTLRME | GGCACCAATGATTATTCACGGCATCAAGACCCAGGGTGCCCGT |
| | LMGCDLNSCSMPLG | CAGAAGTTCTCCAGCCTCTACATCTCTCAGTTTATCATCATGTA |
| | MESKAISDAQITASSY | TAGTCTTGATGGGAAGAAGTGGCAGACTTATCGAGGAAATTCC |
| | FTNMFATWSPSKARL | ACTGGAACCTTAATGGTCTTCTTTGGCAATGTGGATTCATCTG |
| | HLQGRSNAWRPQVN | GGATAAAACACAATATTTTTAACCCTCCAATTATTGCTCGATA |
| | NPKEWLQVDFQKTM | CATCCGTTTGCACCCAACTCATTATAGCATTCGCAGCACTCTTC |
| | KVTGVTTQGVKSLLT | GCATGGAGTTGATGGGCTGTGATTTAAATAGTTGCAGCATGCC |
| | SMYVKEFLISSSQDG | ATTGGGAATGGAGAGTAAAGCAATATCAGATGCACAGATTAC |
| | HQWTLFFQNGKVKV | TGCTTCATCCTACTTTACCAATATGTTTGCCACCTGGTCTCCTT |
| | FQGNQDSFTPVVNSL | CAAAAGCTCGACTTCACCTCCAAGGGAGGAGTAATGCCTGGA |
| | DPPLLTRYLRIHPQSW | GACCTCAGGTGAATAATCCAAAAGAGTGGCTGCAAGTGGACT |
| | VHQIALRMEVLGCEA | TCCAGAAGACAATGAAAGTCACAGGAGTAACTACTCAGGGAG |
| | QDLYGAGSPGAETAE | TAAAATCTCTGCTTACCAGCATGTATGTGAAGGAGTTCCTCAT |
| | QKLISEEDLSPATG | CTCCAGCAGTCAAGATGGCCATCAGTGGACTCTCTTTTTTCAG |
| | | AATGGCAAAGTAAAGGTTTTTCAGGGAAATCAAGACTCCTTCA |
| | | CACCTGTGGTGAACTCTCTAGACCCACCGTTACTGACTCGCTA |
| | | CCTTCGAATTCACCCCCAGAGTTGGGTGCACCAGATTGCCCTG |
| | | AGGATGGAGGTTCTGGGCTGCGAGGCACAGGACCTCTACGGC |
| | | GCCGGATCACCTGGGGCCGAAACGGCCGAACAAAAACTCATC |
| | | TCAGAAGAGGATCTGTCACCTGCAACCGGTTGA |
| pBC0165 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQGAPGSPAG | GAACTGTCATGGGACTATATGCAAGGCGCGCCAGGTTCTCCTG |
| | SPTSTEEGTSESATPE | CTGGCTCCCCCACCTCAACAGAAGAGGGGACAAGCGAAAGCG |
| | SGPGSEPATSGSETPA | CTACGCCTGAGAGTGGCCCTGGCTCTGAGCCAGCCACCTCCGG |
| | SSSDLGELPVDARFPP | CTCTGAAACCCCTGCCTCGAGCAGTGATCTCGGTGAGCTGCCT |
| | RVPKSFPFNTSVVYK | GTGGACGCAAGATTTCCTCCTAGAGTGCCAAAATCTTTTCCAT |
| | KTLFVEFTDHLFNIAK | TCAACACCTCAGTCGTGTACAAAAAGACTCTGTTTGTAGAATT |
| | PRPPWMGLLGPTIQA | CACGGATCACCTTTTCAACATCGCTAAGCCAAGGCCACCCTGG |
| | EVYDTVVITLKNMAS | ATGGGTCTGCTAGGTCCTACCATCCAGGCTGAGGTTTATGATA |
| | HPVSLHAVGVSYWK | CAGTGGTCATTACACTTAAGAACATGGCTTCCCATCCTGTCAG |
| | ASEGAEYDDQTSQRE | TCTTCATGCTGTTGGTGTATCCTACTGGAAAGCTTCTGAGGGA |
| | KEDDKVFPGGSHTYV | GCTGAATATGATGATCAGACCAGTCAAAGGGAGAAAGAAGAT |
| | WQVLKENGPMASDP | GATAAAGTCTTCCCTGGTGGAAGCCATACATATGTCTGGCAGG |
| | LCLTYSYLSHVDLVK | TCCTGAAAGAGAATGGTCCAATGGCCTCTGACCCACTGTGCCT |
| | DLNSGLIGALLVCRE | TACCTACTCATATCTTTCTCATGTGGACCTGGTAAAAGACTTG |
| | GSLAKEKTQTLHKFIL | AATTCAGGCCTCATTGGAGCCCTACTAGTATGTAGAGAAGGGA |
| | LFAVFDEGKSWHSET | GTCTGGCCAAGGAAAAGACACAGACCTTGCACAAATTTATACT |
| | KNSLMQDRDAASAR | ACTTTTTGCTGTATTTGATGAAGGGAAAAGTTGGCACTCAGAA |
| | AWPKMHTVNGYVNR | ACAAAGAACTCCTTGATGCAGGATAGGGATGCTGCATCTGCTC |
| | SLPGLIGCHRKSVYW | GGGCCTGGCCTAAAATGCACACAGTCAATGGTTATGTAAACA |
| | HVIGMGTTPEVHSIFL | GGTCTCTGCCAGGTCTGATTGGATGCCACAGGAAATCAGTCTA |
| | EGHTFLVRNHRQASL | TTGGCATGTGATTGGAATGGGCACCACTCCTGAAGTGCACTCA |
| | EISPITFLTAQTLLMD | ATATTCCTCGAAGGTCACACATTTCTTGTGAGGAACCATCGCC |
| | LGQFLLFCHISSHQHD | AGGCTAGCTTGGAAATCTCGCCAATAACTTTCCTTACTGCTCA |
| | GMEAYVKVDSCPEEP | AACACTCTTGATGGACCTTGGACAGTTTCTACTGTTTTGTCATA |
| | QLRMKNNEEAEDYD | TCTCTTCCCACCAACATGATGGCATGGAAGCTTATGTCAAAGT |
| | DDLTDSEMDVVRFD | AGACAGCTGTCCAGAGGAACCCCAACTACGAATGAAAAATAA |
| | DDNSPSFIQIRSVAKK | TGAAGAAGCGGAAGACTATGATGATGATCTTACTGATTCTGAA |
| | HPKTWVHYIAAEEED | ATGGATGTGGTCAGGTTTGATGATGACAACTCTCCTTCCTTTAT |
| | WDYAPLVLAPDDRS | CCAAATTCGCTCAGTTGCCAAGAAGCATCCTAAAACTTGGGTA |
| | YKSQYLNNGPQRIGR | CATTACATTGCTGCTGAAGAGGAGGACTGGGACTATGCTCCCT |
| | KYKKVRFMAYTDET | TAGTCCTCGCCCCCGATGACAGAAGTTATAAAAGTCAATATTT |
| | FKTREAIQHESGILGP | GAACAATGGCCCTCAGCGGATTGGTAGGAAGTACAAAAAAGT |
| | LLYGEVGDTLLIIFKN | CCGATTTATGGCATACACAGATGAAACCTTTAAGACTCGTGAA |
| | QASRPYNIYPHGITDV | GCTATTCAGCATGAATCAGGAATCTTGGGACCTTTACTTTATG |
| | RPLYSRRLPKGVKHL | GGGAAGTTGGAGACACACTGTTGATTATATTTAAGAATCAAGC |
| | KDFPILPGEIFKYKWT | AAGCAGACCATATAACATCTACCCTCACGGAATCACTGATGTC |
| | VTVEDGPTKSDPRCL | CGTCCTTTGTATTCAAGGAGATTACCAAAAGGTGTAAAACATT |
| | TRYYSSFVNMERDLA | TGAAGGATTTTCCAATTCTGCCAGGAGAAATATTCAAATATAA |
| | SGLIGPLLICYKESVD | ATGGACAGTGACTGTAGAAGATGGGCCAACTAAATCAGATCC |
| | QRGNQIMSDKRNVIL | TCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0183 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGGAPG | GAACTGTCATGGGACTATATGCAAAGTGATCTCGGTGGCGCGC |
| | SPAGSPTSTEEGTSES | CAGGTTCTCCTGCTGGCTCCCCCACCTCAACAGAAGAGGGGAC |
| | ATPESGPGSEPATSGS | AAGCGAAAGCGCTACGCCTGAGAGTGGCCCTGGCTCTGAGCC |
| | ETPASSELPVDARFPP | AGCCACCTCCGGCTCTGAAACCCCTGCCTCGAGCGAGCTGCCT |
| | RVPKSFPFNTSVVYK | GTGGACGCAAGATTTCCTCCTAGAGTGCCAAAATCTTTTCCAT |
| | KTLFVEFTDHLFNIAK | TCAACACCTCAGTCGTGTACAAAAAGACTCTGTTTGTAGAATT |
| | PRPPWMGLLGPTIQA | CACGGATCACCTTTTCAACATCGCTAAGCCAAGGCCACCCTGG |
| | EVYDTVVITLKNMAS | ATGGGTCTGCTAGGTCCTACCATCCAGGCTGAGGTTTATGATA |
| | HPVSLHAVGVSYWK | CAGTGGTCATTACACTTAAGAACATGGCTTCCCATCCTGTCAG |
| | ASEGAEYDDQTSQRE | TCTTCATGCTGTTGGTGTATCCTACTGGAAAGCTTCTGAGGGA |
| | KEDDKVFPGGSHTYV | GCTGAATATGATGATCAGACCAGTCAAAGGGAGAAAGAAGAT |
| | WQVLKENGPMASDP | GATAAAGTCTTCCCTGGTGGAAGCCATACATATGTCTGGCAGG |
| | LCLTYSYLSHVDLVK | TCCTGAAAGAGAATGGTCCAATGGCCTCTGACCCACTGTGCCT |
| | DLNSGLIGALLVCRE | TACCTACTCATATCTTTCTCATGTGGACCTGGTAAAAGACTTG |
| | GSLAKEKTQTLHKFIL | AATTCAGGCCTCATTGGAGCCCTACTAGTATGTAGAGAAGGGA |
| | LFAVFDEGKSWHSET | GTCTGGCCAAGGAAAAGACACAGACCTTGCACAAATTTATACT |
| | KNSLMQDRDAASAR | ACTTTTTGCTGTATTTGATGAAGGGAAAAGTTGGCACTCAGAA |
| | AWPKMHTVNGYVNR | ACAAAGAACTCCTTGATGCAGGATAGGGATGCTGCATCTGCTC |
| | SLPGLIGCHRKSVYW | GGGCCTGGCCTAAAATGCACACAGTCAATGGTTATGTAAACA |
| | HVIGMGTTPEVHSIFL | GGTCTCTGCCAGGTCTGATTGGATGCCACAGGAAATCAGTCTA |
| | EGHTFLVRNHRQASL | TTGGCATGTGATTGGAATGGGCACCACTCCTGAAGTGCACTCA |
| | EISPITFLTAQTLLMD | ATATTCCTCGAAGGTCACACATTTCTTGTGAGGAACCATCGCC |
| | LGQFLLFCHISSHQHD | AGGCTAGCTTGGAAATCTCGCCAATAACTTTCCTTACTGCTCA |
| | GMEAYVKVDSCPEEP | AACACTCTTGATGGACCTTGGACAGTTTCTACTGTTTTGTCATA |
| | QLRMKNNEEAEDYD | TCTCTTCCCACCAACATGATGGCATGGAAGCTTATGTCAAAGT |
| | DDLTDSEMDVVRFD | AGACAGCTGTCCAGAGGAACCCCAACTACGAATGAAAAATAA |
| | DDNSPSFIQIRSVAKK | TGAAGAAGCGGAAGACTATGATGATGATCTTACTGATTCTGAA |
| | HPKTWVHYIAAEEED | ATGGATGTGGTCAGGTTTGATGATGACAACTCTCCTTCCTTTAT |
| | WDYAPLVLAPDDRS | CCAAATTCGCTCAGTTGCCAAGAAGCATCCTAAAACTTGGGTA |
| | YKSQYLNNGPQRIGR | CATTACATTGCTGCTGAAGAGGAGGACTGGGACTATGCTCCCT |
| | KYKKVRFMAYTDET | TAGTCCTCGCCCCCGATGACAGAAGTTATAAAAGTCAATATTT |
| | FKTREAIQHESGILGP | GAACAATGGCCCTCAGCGGATTGGTAGGAAGTACAAAAAAGT |
| | LLYGEVGDTLLIIFKN | CCGATTTATGGCATACACAGATGAAACCTTTAAGACTCGTGAA |
| | QASRPYNIYPHGITDV | GCTATTCAGCATGAATCAGGAATCTTGGGACCTTTACTTTATG |
| | RPLYSRRLPKGVKHL | GGGAAGTTGGAGACACACTGTTGATTATATTTAAGAATCAAGC |
| | KDFPILPGEIFKYKWT | AAGCAGACCATATAACATCTACCCTCACGGAATCACTGATGTC |
| | VTVEDGPTKSDPRCL | CGTCCTTTGTATTCAAGGAGATTACCAAAAGGTGTAAAACATT |
| | TRYYSSFVNMERDLA | TGAAGGATTTTCCAATTCTGCCAGGAGAAATATTCAAATATAA |
| | SGLIGPLLICYKESVD | ATGGACAGTGACTGTAGAAGATGGGCCAACTAAATCAGATCC |
| | QRGNQIMSDKRNVIL | TCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0184 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | GAPGSPAGSPTSTEEG | CTGTGGGCGCGCCAGGTTCTCCTGCTGGCTCCCCCACCTCAAC |
| | TSESATPESGPGSEPA | AGAAGAGGGGACAAGCGAAAGCGCTACGCCTGAGAGTGGCCC |
| | TSGSETPASSDARFPP | TGGCTCTGAGCCAGCCACCTCCGGCTCTGAAACCCCTGCCTCG |
| | RVPKSFPFNTSVVYK | AGCGACGCAAGATTTCCTCCTAGAGTGCCAAAATCTTTTCCAT |
| | KTLFVEFTDHLFNIAK | TCAACACCTCAGTCGTGTACAAAAAGACTCTGTTTGTAGAATT |
| | PRPPWMGLLGPTIQA | CACGGATCACCTTTTCAACATCGCTAAGCCAAGGCCACCCTGG |
| | EVYDTVVITLKNMAS | ATGGGTCTGCTAGGTCCTACCATCCAGGCTGAGGTTTATGATA |
| | HPVSLHAVGVSYWK | CAGTGGTCATTACACTTAAGAACATGGCTTCCCATCCTGTCAG |
| | ASEGAEYDDQTSQRE | TCTTCATGCTGTTGGTGTATCCTACTGGAAAGCTTCTGAGGGA |
| | KEDDKVFPGGSHTYV | GCTGAATATGATGATCAGACCAGTCAAAGGGAGAAAGAAGAT |
| | WQVLKENGPMASDP | GATAAAGTCTTCCCTGGTGGAAGCCATACATATGTCTGGCAGG |
| | LCLTYSYLSHVDLVK | TCCTGAAAGAGAATGGTCCAATGGCCTCTGACCCACTGTGCCT |
| | DLNSGLIGALLVCRE | TACCTACTCATATCTTTCTCATGTGGACCTGGTAAAAGACTTG |
| | GSLAKEKTQTLHKFIL | AATTCAGGCCTCATTGGAGCCCTACTAGTATGTAGAGAAGGGA |
| | LFAVFDEGKSWHSET | GTCTGGCCAAGGAAAAGACACAGACCTTGCACAAATTTATACT |
| | KNSLMQDRDAASAR | ACTTTTTGCTGTATTTGATGAAGGGAAAAGTTGGCACTCAGAA |
| | AWPKMHTVNGYVNR | ACAAAGAACTCCTTGATGCAGGATAGGGATGCTGCATCTGCTC |
| | SLPGLIGCHRKSVYW | GGGCCTGGCCTAAAATGCACACAGTCAATGGTTATGTAAACA |
| | HVIGMGTTPEVHSIFL | GGTCTCTGCCAGGTCTGATTGGATGCCACAGGAAATCAGTCTA |
| | EGHTFLVRNHRQASL | TTGGCATGTGATTGGAATGGGCACCACTCCTGAAGTGCACTCA |
| | EISPITFLTAQTLLMD | ATATTCCTCGAAGGTCACACATTTCTTGTGAGGAACCATCGCC |
| | LGQFLLFCHISSHQHD | AGGCTAGCTTGGAAATCTCGCCAATAACTTTCCTTACTGCTCA |
| | GMEAYVKVDSCPEEP | AACACTCTTGATGGACCTTGGACAGTTTCTACTGTTTTGTCATA |
| | QLRMKNNEEAEDYD | TCTCTTCCCACCAACATGATGGCATGGAAGCTTATGTCAAAGT |
| | DDLTDSEMDVVRFD | AGACAGCTGTCCAGAGGAACCCCAACTACGAATGAAAAATAA |
| | DDNSPSFIQIRSVAKK | TGAAGAAGCGGAAGACTATGATGATGATCTTACTGATTCTGAA |
| | HPKTWVHYIAAEEED | ATGGATGTGGTCAGGTTTGATGATGACAACTCTCCTTCCTTTAT |
| | WDYAPLVLAPDDRS | CCAAATTCGCTCAGTTGCCAAGAAGCATCCTAAAACTTGGGTA |
| | YKSQYLNNGPQRIGR | CATTACATTGCTGCTGAAGAGGAGGACTGGGACTATGCTCCCT |
| | KYKKVRFMAYTDET | TAGTCCTCGCCCCCGATGACAGAAGTTATAAAAGTCAATATTT |
| | FKTREAIQHESGILGP | GAACAATGGCCCTCAGCGGATTGGTAGGAAGTACAAAAAAGT |
| | LLYGEVGDTLLIIFKN | CCGATTTATGGCATACACAGATGAAACCTTTAAGACTCGTGAA |
| | QASRPYNIYPHGITDV | GCTATTCAGCATGAATCAGGAATCTTGGGACCTTTACTTTATG |
| | RPLYSRRLPKGVKHL | GGGAAGTTGGAGACACACTGTTGATTATATTTAAGAATCAAGC |
| | KDFPILPGEIFKYKWT | AAGCAGACCATATAACATCTACCCTCACGGAATCACTGATGTC |
| | VTVEDGPTKSDPRCL | CGTCCTTTGTATTCAAGGAGATTACCAAAAGGTGTAAAACATT |
| | TRYYSSFVNMERDLA | TGAAGGATTTTCCAATTCTGCCAGGAGAAATATTCAAATATAA |
| | SGLIGPLLICYKESVD | ATGGACAGTGACTGTAGAAGATGGGCCAACTAAATCAGATCC |
| | QRGNQIMSDKRNVIL | TCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0166 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFGA | CTGTGGACGCAAGATTTCCTCCTAGAGTGCCAAAATCTTTTCC |
| | PGSPAGSPTSTEEGTS | ATTCGGCGCGCCAGGTTCTCCTGCTGGCTCCCCCACCTCAACA |
| | ESATPESGPGSEPATS | GAAGAGGGGACAAGCGAAAGCGCTACGCCTGAGAGTGGCCCT |
| | GSETPASSNTSVVYK | GGCTCTGAGCCAGCCACCTCCGGCTCTGAAACCCCTGCCTCGA |
| | KTLFVEFTDHLFNIAK | GCAACACCTCAGTCGTGTACAAAAAGACTCTGTTTGTAGAATT |
| | PRPPWMGLLGPTIQA | CACGGATCACCTTTTCAACATCGCTAAGCCAAGGCCACCCTGG |
| | EVYDTVVITLKNMAS | ATGGGTCTGCTAGGTCCTACCATCCAGGCTGAGGTTTATGATA |
| | HPVSLHAVGVSYWK | CAGTGGTCATTACACTTAAGAACATGGCTTCCCATCCTGTCAG |
| | ASEGAEYDDQTSQRE | TCTTCATGCTGTTGGTGTATCCTACTGGAAAGCTTCTGAGGGA |
| | KEDDKVFPGGSHTYV | GCTGAATATGATGATCAGACCAGTCAAAGGGAGAAAGAAGAT |
| | WQVLKENGPMASDP | GATAAAGTCTTCCCTGGTGGAAGCCATACATATGTCTGGCAGG |
| | LCLTYSYLSHVDLVK | TCCTGAAAGAGAATGGTCCAATGGCCTCTGACCCACTGTGCCT |
| | DLNSGLIGALLVCRE | TACCTACTCATATCTTTCTCATGTGGACCTGGTAAAAGACTTG |
| | GSLAKEKTQTLHKFIL | AATTCAGGCCTCATTGGAGCCCTACTAGTATGTAGAGAAGGGA |
| | LFAVFDEGKSWHSET | GTCTGGCCAAGGAAAAGACACAGACCTTGCACAAATTTATACT |
| | KNSLMQDRDAASAR | ACTTTTTGCTGTATTTGATGAAGGGAAAAGTTGGCACTCAGAA |
| | AWPKMHTVNGYVNR | ACAAAGAACTCCTTGATGCAGGATAGGGATGCTGCATCTGCTC |
| | SLPGLIGCHRKSVYW | GGGCCTGGCCTAAAATGCACACAGTCAATGGTTATGTAAACA |
| | HVIGMGTTPEVHSIFL | GGTCTCTGCCAGGTCTGATTGGATGCCACAGGAAATCAGTCTA |
| | EGHTFLVRNHRQASL | TTGGCATGTGATTGGAATGGGCACCACTCCTGAAGTGCACTCA |
| | EISPITFLTAQTLLMD | ATATTCCTCGAAGGTCACACATTTCTTGTGAGGAACCATCGCC |
| | LGQFLLFCHISSHQHD | AGGCTAGCTTGGAAATCTCGCCAATAACTTTCCTTACTGCTCA |
| | GMEAYVKVDSCPEEP | AACACTCTTGATGGACCTTGGACAGTTTCTACTGTTTTGTCATA |
| | QLRMKNNEEAEDYD | TCTCTTCCCACCAACATGATGGCATGGAAGCTTATGTCAAAGT |
| | DDLTDSEMDVVRFD | AGACAGCTGTCCAGAGGAACCCCAACTACGAATGAAAAATAA |
| | DDNSPSFIQIRSVAKK | TGAAGAAGCGGAAGACTATGATGATGATCTTACTGATTCTGAA |
| | HPKTWVHYIAAEEED | ATGGATGTGGTCAGGTTTGATGATGACAACTCTCCTTCCTTTAT |
| | WDYAPLVLAPDDRS | CCAAATTCGCTCAGTTGCCAAGAAGCATCCTAAAACTTGGGTA |
| | YKSQYLNNGPQRIGR | CATTACATTGCTGCTGAAGAGGAGGACTGGGACTATGCTCCCT |
| | KYKKVRFMAYTDET | TAGTCCTCGCCCCCGATGACAGAAGTTATAAAAGTCAATATTT |
| | FKTREAIQHESGILGP | GAACAATGGCCCTCAGCGGATTGGTAGGAAGTACAAAAAAGT |
| | LLYGEVGDTLLIIFKN | CCGATTTATGGCATACACAGATGAAACCTTTAAGACTCGTGAA |
| | QASRPYNIYPHGITDV | GCTATTCAGCATGAATCAGGAATCTTGGGACCTTTACTTTATG |
| | RPLYSRRLPKGVKHL | GGGAAGTTGGAGACACACTGTTGATTATATTTAAGAATCAAGC |
| | KDFPILPGEIFKYKWT | AAGCAGACCATATAACATCTACCCTCACGGAATCACTGATGTC |
| | VTVEDGPTKSDPRCL | CGTCCTTTGTATTCAAGGAGATTACCAAAAGGTGTAAAACATT |
| | TRYYSSFVNMERDLA | TGAAGGATTTTCCAATTCTGCCAGGAGAAATATTCAAATATAA |
| | SGLIGPLLICYKESVD | ATGGACAGTGACTGTAGAAGATGGGCCAACTAAATCAGATCC |
| | QRGNQIMSDKRNVIL | TCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0185 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNGAPGSPAGSPTST | TTCACGGATCACCTTTTCAACGGCGCGCCAGGTTCTCCTGCTG |
| | EEGTSESATPESGPGS | GCTCCCCCACCTCAACAGAAGAGGGGACAAGCGAAAGCGCTA |
| | EPATSGSETPASSIAK | CGCCTGAGAGTGGCCCTGGCTCTGAGCCAGCCACCTCCGGCTC |
| | PRPPWMGLLGPTIQA | TGAAACCCCTGCCTCGAGCATCGCTAAGCCAAGGCCACCCTGG |
| | EVYDTVVITLKNMAS | ATGGGTCTGCTAGGTCCTACCATCCAGGCTGAGGTTTATGATA |
| | HPVSLHAVGVSYWK | CAGTGGTCATTACACTTAAGAACATGGCTTCCCATCCTGTCAG |
| | ASEGAEYDDQTSQRE | TCTTCATGCTGTTGGTGTATCCTACTGGAAAGCTTCTGAGGGA |
| | KEDDKVFPGGSHTYV | GCTGAATATGATGATCAGACCAGTCAAAGGGAGAAAGAAGAT |
| | WQVLKENGPMASDP | GATAAAGTCTTCCCTGGTGGAAGCCATACATATGTCTGGCAGG |
| | LCLTYSYLSHVDLVK | TCCTGAAAGAGAATGGTCCAATGGCCTCTGACCCACTGTGCCT |
| | DLNSGLIGALLVCRE | TACCTACTCATATCTTTCTCATGTGGACCTGGTAAAAGACTTG |
| | GSLAKEKTQTLHKFIL | AATTCAGGCCTCATTGGAGCCCTACTAGTATGTAGAGAAGGGA |
| | LFAVFDEGKSWHSET | GTCTGGCCAAGGAAAAGACACAGACCTTGCACAAATTTATACT |
| | KNSLMQDRDAASAR | ACTTTTTGCTGTATTTGATGAAGGGAAAAGTTGGCACTCAGAA |
| | AWPKMHTVNGYVNR | ACAAAGAACTCCTTGATGCAGGATAGGGATGCTGCATCTGCTC |
| | SLPGLIGCHRKSVYW | GGGCCTGGCCTAAAATGCACACAGTCAATGGTTATGTAAACA |
| | HVIGMGTTPEVHSIFL | GGTCTCTGCCAGGTCTGATTGGATGCCACAGGAAATCAGTCTA |
| | EGHTFLVRNHRQASL | TTGGCATGTGATTGGAATGGGCACCACTCCTGAAGTGCACTCA |
| | EISPITFLTAQTLLMD | ATATTCCTCGAAGGTCACACATTTCTTGTGAGGAACCATCGCC |
| | LGQFLLFCHISSHQHD | AGGCTAGCTTGGAAATCTCGCCAATAACTTTCCTTACTGCTCA |
| | GMEAYVKVDSCPEEP | AACACTCTTGATGGACCTTGGACAGTTTCTACTGTTTTGTCATA |
| | QLRMKNNEEAEDYD | TCTCTTCCCACCAACATGATGGCATGGAAGCTTATGTCAAAGT |
| | DDLTDSEMDVVRFD | AGACAGCTGTCCAGAGGAACCCCAACTACGAATGAAAAATAA |
| | DDNSPSFIQIRSVAKK | TGAAGAAGCGGAAGACTATGATGATGATCTTACTGATTCTGAA |
| | HPKTWVHYIAAEEED | ATGGATGTGGTCAGGTTTGATGATGACAACTCTCCTTCCTTTAT |
| | WDYAPLVLAPDDRS | CCAAATTCGCTCAGTTGCCAAGAAGCATCCTAAAACTTGGGTA |
| | YKSQYLNNGPQRIGR | CATTACATTGCTGCTGAAGAGGAGGACTGGGACTATGCTCCCT |
| | KYKKVRFMAYTDET | TAGTCCTCGCCCCCGATGACAGAAGTTATAAAAGTCAATATTT |
| | FKTREAIQHESGILGP | GAACAATGGCCCTCAGCGGATTGGTAGGAAGTACAAAAAAGT |
| | LLYGEVGDTLLIIFKN | CCGATTTATGGCATACACAGATGAAACCTTTAAGACTCGTGAA |
| | QASRPYNIYPHGITDV | GCTATTCAGCATGAATCAGGAATCTTGGGACCTTTACTTTATG |
| | RPLYSRRLPKGVKHL | GGGAAGTTGGAGACACACTGTTGATTATATTTAAGAATCAAGC |
| | KDFPILPGEIFKYKWT | AAGCAGACCATATAACATCTACCCTCACGGAATCACTGATGTC |
| | VTVEDGPTKSDPRCL | CGTCCTTTGTATTCAAGGAGATTACCAAAAGGTGTAAAACATT |
| | TRYYSSFVNMERDLA | TGAAGGATTTTCCAATTCTGCCAGGAGAAATATTCAAATATAA |
| | SGLIGPLLICYKESVD | ATGGACAGTGACTGTAGAAGATGGGCCAACTAAATCAGATCC |
| | QRGNQIMSDKRNVIL | TCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0167 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | GAPGSPAGSPTSTEEG | GAGCTGAATATGATGATGGCGCGCCAGGTTCTCCTGCTGGCTC |
| | TSESATPESGPGSEPA | CCCCACCTCAACAGAAGAGGGGACAAGCGAAAGCGCTACGCC |
| | TSGSETPASSQTSQRE | TGAGAGTGGCCCTGGCTCTGAGCCAGCCACCTCCGGCTCTGAA |
| | KEDDKVFPGGSHTYV | ACCCCTGCCTCGAGCCAGACCAGTCAAAGGGAGAAAGAAGAT |
| | WQVLKENGPMASDP | GATAAAGTCTTCCCTGGTGGAAGCCATACATATGTCTGGCAGG |
| | LCLTYSYLSHVDLVK | TCCTGAAAGAGAATGGTCCAATGGCCTCTGACCCACTGTGCCT |
| | DLNSGLIGALLVCRE | TACCTACTCATATCTTTCTCATGTGGACCTGGTAAAAGACTTG |
| | GSLAKEKTQTLHKFIL | AATTCAGGCCTCATTGGAGCCCTACTAGTATGTAGAGAAGGGA |
| | LFAVFDEGKSWHSET | GTCTGGCCAAGGAAAAGACACAGACCTTGCACAAATTTATACT |
| | KNSLMQDRDAASAR | ACTTTTTGCTGTATTTGATGAAGGGAAAAGTTGGCACTCAGAA |
| | AWPKMHTVNGYVNR | ACAAAGAACTCCTTGATGCAGGATAGGGATGCTGCATCTGCTC |
| | SLPGLIGCHRKSVYW | GGGCCTGGCCTAAAATGCACACAGTCAATGGTTATGTAAACA |
| | HVIGMGTTPEVHSIFL | GGTCTCTGCCAGGTCTGATTGGATGCCACAGGAAATCAGTCTA |
| | EGHTFLVRNHRQASL | TTGGCATGTGATTGGAATGGGCACCACTCCTGAAGTGCACTCA |
| | EISPITFLTAQTLLMD | ATATTCCTCGAAGGTCACACATTTCTTGTGAGGAACCATCGCC |
| | LGQFLLFCHISSHQHD | AGGCTAGCTTGGAAATCTCGCCAATAACTTTCCTTACTGCTCA |
| | GMEAYVKVDSCPEEP | AACACTCTTGATGGACCTTGGACAGTTTCTACTGTTTTGTCATA |
| | QLRMKNNEEAEDYD | TCTCTTCCCACCAACATGATGGCATGGAAGCTTATGTCAAAGT |
| | DDLTDSEMDVVRFD | AGACAGCTGTCCAGAGGAACCCCAACTACGAATGAAAAATAA |
| | DDNSPSFIQIRSVAKK | TGAAGAAGCGGAAGACTATGATGATGATCTTACTGATTCTGAA |
| | HPKTWVHYIAAEEED | ATGGATGTGGTCAGGTTTGATGATGACAACTCTCCTTCCTTTAT |
| | WDYAPLVLAPDDRS | CCAAATTCGCTCAGTTGCCAAGAAGCATCCTAAAACTTGGGTA |
| | YKSQYLNNGPQRIGR | CATTACATTGCTGCTGAAGAGGAGGACTGGGACTATGCTCCCT |
| | KYKKVRFMAYTDET | TAGTCCTCGCCCCCGATGACAGAAGTTATAAAAGTCAATATTT |
| | FKTREAIQHESGILGP | GAACAATGGCCCTCAGCGGATTGGTAGGAAGTACAAAAAAGT |
| | LLYGEVGDTLLIIFKN | CCGATTTATGGCATACACAGATGAAACCTTTAAGACTCGTGAA |
| | QASRPYNIYPHGITDV | GCTATTCAGCATGAATCAGGAATCTTGGGACCTTTACTTTATG |
| | RPLYSRRLPKGVKHL | GGGAAGTTGGAGACACACTGTTGATTATATTTAAGAATCAAGC |
| | KDFPILPGEIFKYKWT | AAGCAGACCATATAACATCTACCCTCACGGAATCACTGATGTC |
| | VTVEDGPTKSDPRCL | CGTCCTTTGTATTCAAGGAGATTACCAAAAGGTGTAAAACATT |
| | TRYYSSFVNMERDLA | TGAAGGATTTTCCAATTCTGCCAGGAGAAATATTCAAATATAA |
| | SGLIGPLLICYKESVD | ATGGACAGTGACTGTAGAAGATGGGCCAACTAAATCAGATCC |
| | QRGNQIMSDKRNVIL | TCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0128 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | APGSPAGSPTSTEEGT | ATGATAAAGTCTTCCCTGGCGCGCCAGGTTCTCCTGCTGGCTC |
| | SESATPESGPGSEPAT | CCCCACCTCAACAGAAGAGGGGACAAGCGAAAGCGCTACGCC |
| | SGSETPASSGGSHTY | TGAGAGTGGCCCTGGCTCTGAGCCAGCCACCTCCGGCTCTGAA |
| | VWQVLKENGPMASD | ACCCCTGCCTCGAGCGGTGGAAGCCATACATATGTCTGGCAGG |
| | PLCLTYSYLSHVDLV | TCCTGAAAGAGAATGGTCCAATGGCCTCTGACCCACTGTGCCT |
| | KDLNSGLIGALLVCR | TACCTACTCATATCTTTCTCATGTGGACCTGGTAAAAGACTTG |
| | EGSLAKEKTQTLHKFI | AATTCAGGCCTCATTGGAGCCCTACTAGTATGTAGAGAAGGGA |
| | LLFAVFDEGKSWHSE | GTCTGGCCAAGGAAAAGACACAGACCTTGCACAAATTTATACT |
| | TKNSLMQDRDAASA | ACTTTTTGCTGTATTTGATGAAGGGAAAAGTTGGCACTCAGAA |
| | RAWPKMHTVNGYVN | ACAAAGAACTCCTTGATGCAGGATAGGGATGCTGCATCTGCTC |
| | RSLPGLIGCHRKSVY | GGGCCTGGCCTAAAATGCACACAGTCAATGGTTATGTAAACA |
| | WHVIGMGTTPEVHSI | GGTCTCTGCCAGGTCTGATTGGATGCCACAGGAAATCAGTCTA |
| | FLEGHTFLVRNHRQA | TTGGCATGTGATTGGAATGGGCACCACTCCTGAAGTGCACTCA |
| | SLEISPITFLTAQTLLM | ATATTCCTCGAAGGTCACACATTTCTTGTGAGGAACCATCGCC |
| | DLGQFLLFCHISSHQH | AGGCTAGCTTGGAAATCTCGCCAATAACTTTCCTTACTGCTCA |
| | DGMEAYVKVDSCPE | AACACTCTTGATGGACCTTGGACAGTTTCTACTGTTTTGTCATA |
| | EPQLRMKNNEEAED | TCTCTTCCCACCAACATGATGGCATGGAAGCTTATGTCAAAGT |
| | YDDDLTDSEMDVVR | AGACAGCTGTCCAGAGGAACCCCAACTACGAATGAAAAATAA |
| | FDDDNSPSFIQIRSVA | TGAAGAAGCGGAAGACTATGATGATGATCTTACTGATTCTGAA |
| | KKHPKTWVHYIAAEE | ATGGATGTGGTCAGGTTTGATGATGACAACTCTCCTTCCTTTAT |
| | EDWDYAPLVLAPDD | CCAAATTCGCTCAGTTGCCAAGAAGCATCCTAAAACTTGGGTA |
| | RSYKSQYLNNGPQRI | CATTACATTGCTGCTGAAGAGGAGGACTGGGACTATGCTCCCT |
| | GRKYKKVRFMAYTD | TAGTCCTCGCCCCCGATGACAGAAGTTATAAAAGTCAATATTT |
| | ETFKTREAIQHESGIL | GAACAATGGCCCTCAGCGGATTGGTAGGAAGTACAAAAAAGT |
| | GPLLYGEVGDTLLIIF | CCGATTTATGGCATACACAGATGAAACCTTTAAGACTCGTGAA |
| | KNQASRPYNIYPHGIT | GCTATTCAGCATGAATCAGGAATCTTGGGACCTTTACTTTATG |
| | DVRPLYSRRLPKGVK | GGGAAGTTGGAGACACACTGTTGATTATATTTAAGAATCAAGC |
| | HLKDFPILPGEIFKYK | AAGCAGACCATATAACATCTACCCTCACGGAATCACTGATGTC |
| | WTVTVEDGPTKSDPR | CGTCCTTTGTATTCAAGGAGATTACCAAAAGGTGTAAAACATT |
| | CLTRYYSSFVNMERD | TGAAGGATTTTCCAATTCTGCCAGGAGAAATATTCAAATATAA |
| | LASGLIGPLLICYKES | ATGGACAGTGACTGTAGAAGATGGGCCAACTAAATCAGATCC |
| | VDQRGNQIMSDKRN | TCGGTGCCTGACCCGCTATTACTCTAGTTTCGTTAATATGGAG |
| | VILFSVFDENRSWYL | AGAGATCTAGCTTCAGGACTCATTGGCCCTCTCCTCATCTGCT |
| | TENIQRFLPNPAGVQL | ACAAAGAATCTGTAGATCAAAGAGGAAACCAGATAATGTCAG |
| | EDPEFQASNIMHSING | ACAAGAGGAATGTCATCCTGTTTTCTGTATTTGATGAGAACCG |
| | YVFDSLQLSVCLHEV | AAGCTGGTACCTCACAGAGAATATACAACGCTTTCTCCCCAAT |
| | AYWYILSIGAQTDFLS | CCAGCTGGAGTGCAGCTTGAGGATCCAGAGTTCCAAGCCTCCA |
| | VFFSGYTFKHKMVYE | ACATCATGCACAGCATCAATGGCTATGTTTTTGATAGTTTGCA |
| | DTLTLFPFSGETVFMS | GTTGTCAGTTTGTTTGCATGAGGTGGCATACTGGTACATTCTA |
| | MENPGLWILGCHNSD | AGCATTGGAGCACAGACTGACTTCCTTTCTGTCTTCTTCTCTGG |
| | FRNRGMTALLKVSSC | ATATACCTTCAAACACAAAATGGTCTATGAAGACACACTCACC |
| | DKNTGDYYEDSYEDI | CTATTCCCATTCTCAGGAGAAACTGTCTTCATGTCGATGGAAA |
| | SAYLLSKNNAIEPRSF | ACCCAGGTCTATGGATTCTGGGGTGCCACAACTCAGACTTTCG |
| | SQNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0168 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKGA | GAATTCAGGCCTCATTGGAGCCCTACTAGTATGTAGAGAAGGG |
| | PGSPAGSPTSTEEGTS | AGTCTGGCCAAGGAAAAGGGCGCGCCAGGTTCTCCTGCTGGCT |
| | ESATPESGPGSEPATS | CCCCCACCTCAACAGAAGAGGGGACAAGCGAAAGCGCTACGC |
| | GSETPASSTQTLHKFI | CTGAGAGTGGCCCTGGCTCTGAGCCAGCCACCTCCGGCTCTGA |
| | LLFAVFDEGKSWHSE | AACCCCTGCCTCGAGCACACAGACCTTGCACAAATTTATACTA |
| | TKNSLMQDRDAASA | CTTTTTGCTGTATTTGATGAAGGGAAAAGTTGGCACTCAGAAA |
| | RAWPKMHTVNGYVN | CAAAGAACTCCTTGATGCAGGATAGGGATGCTGCATCTGCTCG |
| | RSLPGLIGCHRKSVY | GGCCTGGCCTAAAATGCACACAGTCAATGGTTATGTAAACAG |
| | WHVIGMGTTPEVHSI | GTCTCTGCCAGGTCTGATTGGATGCCACAGGAAATCAGTCTAT |
| | FLEGHTFLVRNHRQA | TGGCATGTGATTGGAATGGGCACCACTCCTGAAGTGCACTCAA |
| | SLEISPITFLTAQTLLM | TATTCCTCGAAGGTCACACATTTCTTGTGAGGAACCATCGCCA |
| | DLGQFLLFCHISSHQH | GGCTAGCTTGGAAATCTCGCCAATAACTTTCCTTACTGCTCAA |
| | DGMEAYVKVDSCPE | ACACTCTTGATGGACCTTGGACAGTTTCTACTGTTTTGTCATAT |
| | EPQLRMKNNEEAED | CTCTTCCCACCAACATGATGGCATGGAAGCTTATGTCAAAGTA |
| | YDDDLTDSEMDVVR | GACAGCTGTCCAGAGGAACCCCAACTACGAATGAAAAATAAT |
| | FDDDNSPSFIQIRSVA | GAAGAAGCGGAAGACTATGATGATGATCTTACTGATTCTGAA |
| | KKHPKTWVHYIAAEE | ATGGATGTGGTCAGGTTTGATGATGACAACTCTCCTTCCTTTAT |
| | EDWDYAPLVLAPDD | CCAAATTCGCTCAGTTGCCAAGAAGCATCCTAAAACTTGGGTA |
| | RSYKSQYLNNGPQRI | CATTACATTGCTGCTGAAGAGGAGGACTGGGACTATGCTCCCT |
| | GRKYKKVRFMAYTD | TAGTCCTCGCCCCCGATGACAGAAGTTATAAAAGTCAATATTT |
| | ETFKTREAIQHESGIL | GAACAATGGCCCTCAGCGGATTGGTAGGAAGTACAAAAAAGT |
| | GPLLYGEVGDTLLIIF | CCGATTTATGGCATACACAGATGAAACCTTTAAGACTCGTGAA |
| | KNQASRPYNIYPHGIT | GCTATTCAGCATGAATCAGGAATCTTGGGACCTTTACTTTATG |
| | DVRPLYSRRLPKGVK | GGGAAGTTGGAGACACACTGTTGATTATATTTAAGAATCAAGC |
| | HLKDFPILPGEIFKYK | AAGCAGACCATATAACATCTACCCTCACGGAATCACTGATGTC |
| | WTVTVEDGPTKSDPR | CGTCCTTTGTATTCAAGGAGATTACCAAAAGGTGTAAAACATT |
| | CLTRYYSSFVNMERD | TGAAGGATTTTCCAATTCTGCCAGGAGAAATATTCAAATATAA |
| | LASGLIGPLLICYKES | ATGGACAGTGACTGTAGAAGATGGGCCAACTAAATCAGATCC |
| | VDQRGNQIMSDKRN | TCGGTGCCTGACCCGCTATTACTCTAGTTTCGTTAATATGGAG |
| | VILFSVFDENRSWYL | AGAGATCTAGCTTCAGGACTCATTGGCCCTCTCCTCATCTGCT |
| | TENIQRFLPNPAGVQL | ACAAAGAATCTGTAGATCAAAGAGGAAACCAGATAATGTCAG |
| | EDPEFQASNIMHSING | ACAAGAGGAATGTCATCCTGTTTTCTGTATTTGATGAGAACCG |
| | YVFDSLQLSVCLHEV | AAGCTGGTACCTCACAGAGAATATACAACGCTTTCTCCCCAAT |
| | AYWYILSIGAQTDFLS | CCAGCTGGAGTGCAGCTTGAGGATCCAGAGTTCCAAGCCTCCA |
| | VFFSGYTFKHKMVYE | ACATCATGCACAGCATCAATGGCTATGTTTTTGATAGTTTGCA |
| | DTLTLFPFSGETVFMS | GTTGTCAGTTTGTTTGCATGAGGTGGCATACTGGTACATTCTA |
| | MENPGLWILGCHNSD | AGCATTGGAGCACAGACTGACTTCCTTTCTGTCTTCTTCTCTGG |
| | FRNRGMTALLKVSSC | ATATACCTTCAAACACAAAATGGTCTATGAAGACACACTCACC |
| | DKNTGDYYEDSYEDI | CTATTCCCATTCTCAGGAGAAACTGTCTTCATGTCGATGGAAA |
| | SAYLLSKNNAIEPRSF | ACCCAGGTCTATGGATTCTGGGGTGCCACAACTCAGACTTTCG |
| | SQNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0129 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLGAPGS | CTACTTTTTGCTGTATTTGATGAAGGGAAAAGTTGGCACTCAG |
| | PAGSPTSTEEGTSESA | AAACAAAGAACTCCTTGGGCGCGCCAGGTTCTCCTGCTGGCTC |
| | TPESGPGSEPATSGSE | CCCCACCTCAACAGAAGAGGGGACAAGCGAAAGCGCTACGCC |
| | TPASSMQDRDAASAR | TGAGAGTGGCCCTGGCTCTGAGCCAGCCACCTCCGGCTCTGAA |
| | AWPKMHTVNGYVNR | ACCCCTGCCTCGAGCATGCAGGATAGGGATGCTGCATCTGCTC |
| | SLPGLIGCHRKSVYW | GGGCCTGGCCTAAAATGCACACAGTCAATGGTTATGTAAACA |
| | HVIGMGTTPEVHSIFL | GGTCTCTGCCAGGTCTGATTGGATGCCACAGGAAATCAGTCTA |
| | EGHTFLVRNHRQASL | TTGGCATGTGATTGGAATGGGCACCACTCCTGAAGTGCACTCA |
| | EISPITFLTAQTLLMD | ATATTCCTCGAAGGTCACACATTTCTTGTGAGGAACCATCGCC |
| | LGQFLLFCHISSHQHD | AGGCTAGCTTGGAAATCTCGCCAATAACTTTCCTTACTGCTCA |
| | GMEAYVKVDSCPEEP | AACACTCTTGATGGACCTTGGACAGTTTCTACTGTTTTGTCATA |
| | QLRMKNNEEAEDYD | TCTCTTCCCACCAACATGATGGCATGGAAGCTTATGTCAAAGT |
| | DDLTDSEMDVVRFD | AGACAGCTGTCCAGAGGAACCCCAACTACGAATGAAAAATAA |
| | DDNSPSFIQIRSVAKK | TGAAGAAGCGGAAGACTATGATGATGATCTTACTGATTCTGAA |
| | HPKTWVHYIAAEEED | ATGGATGTGGTCAGGTTTGATGATGACAACTCTCCTTCCTTTAT |
| | WDYAPLVLAPDDRS | CCAAATTCGCTCAGTTGCCAAGAAGCATCCTAAAACTTGGGTA |
| | YKSQYLNNGPQRIGR | CATTACATTGCTGCTGAAGAGGAGGACTGGGACTATGCTCCCT |
| | KYKKVRFMAYTDET | TAGTCCTCGCCCCCGATGACAGAAGTTATAAAAGTCAATATTT |
| | FKTREAIQHESGILGP | GAACAATGGCCCTCAGCGGATTGGTAGGAAGTACAAAAAAGT |
| | LLYGEVGDTLLIIFKN | CCGATTTATGGCATACACAGATGAAACCTTTAAGACTCGTGAA |
| | QASRPYNIYPHGITDV | GCTATTCAGCATGAATCAGGAATCTTGGGACCTTTACTTTATG |
| | RPLYSRRLPKGVKHL | GGGAAGTTGGAGACACACTGTTGATTATATTTAAGAATCAAGC |
| | KDFPILPGEIFKYKWT | AAGCAGACCATATAACATCTACCCTCACGGAATCACTGATGTC |
| | VTVEDGPTKSDPRCL | CGTCCTTTGTATTCAAGGAGATTACCAAAAGGTGTAAAACATT |
| | TRYYSSFVNMERDLA | TGAAGGATTTTCCAATTCTGCCAGGAGAAATATTCAAATATAA |
| | SGLIGPLLICYKESVD | ATGGACAGTGACTGTAGAAGATGGGCCAACTAAATCAGATCC |
| | QRGNQIMSDKRNVIL | TCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0169 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKGAPG | AAACAAAGAACTCCTTGATGCAGGATAGGGATGCTGCATCTG |
| | SPAGSPTSTEEGTSES | CTCGGGCCTGGCCTAAAGGCGCGCCAGGTTCTCCTGCTGGCTC |
| | ATPESGPGSEPATSGS | CCCCACCTCAACAGAAGAGGGGACAAGCGAAAGCGCTACGCC |
| | ETPASSMHTVNGYVN | TGAGAGTGGCCCTGGCTCTGAGCCAGCCACCTCCGGCTCTGAA |
| | RSLPGLIGCHRKSVY | ACCCCTGCCTCGAGCATGCACACAGTCAATGGTTATGTAAACA |
| | WHVIGMGTTPEVHSI | GGTCTCTGCCAGGTCTGATTGGATGCCACAGGAAATCAGTCTA |
| | FLEGHTFLVRNHRQA | TTGGCATGTGATTGGAATGGGCACCACTCCTGAAGTGCACTCA |
| | SLEISPITFLTAQTLLM | ATATTCCTCGAAGGTCACACATTTCTTGTGAGGAACCATCGCC |
| | DLGQFLLFCHISSHQH | AGGCTAGCTTGGAAATCTCGCCAATAACTTTCCTTACTGCTCA |
| | DGMEAYVKVDSCPE | AACACTCTTGATGGACCTTGGACAGTTTCTACTGTTTTGTCATA |
| | EPQLRMKNNEEAED | TCTCTTCCCACCAACATGATGGCATGGAAGCTTATGTCAAAGT |
| | YDDDLTDSEMDVVR | AGACAGCTGTCCAGAGGAACCCCAACTACGAATGAAAAATAA |
| | FDDDNSPSFIQIRSVA | TGAAGAAGCGGAAGACTATGATGATGATCTTACTGATTCTGAA |
| | KKHPKTWVHYIAAEE | ATGGATGTGGTCAGGTTTGATGATGACAACTCTCCTTCCTTTAT |
| | EDWDYAPLVLAPDD | CCAAATTCGCTCAGTTGCCAAGAAGCATCCTAAAACTTGGGTA |
| | RSYKSQYLNNGPQRI | CATTACATTGCTGCTGAAGAGGAGGACTGGGACTATGCTCCCT |
| | GRKYKKVRFMAYTD | TAGTCCTCGCCCCCGATGACAGAAGTTATAAAAGTCAATATTT |
| | ETFKTREAIQHESGIL | GAACAATGGCCCTCAGCGGATTGGTAGGAAGTACAAAAAAGT |
| | GPLLYGEVGDTLLIIF | CCGATTTATGGCATACACAGATGAAACCTTTAAGACTCGTGAA |
| | KNQASRPYNIYPHGIT | GCTATTCAGCATGAATCAGGAATCTTGGGACCTTTACTTTATG |
| | DVRPLYSRRLPKGVK | GGGAAGTTGGAGACACACTGTTGATTATATTTAAGAATCAAGC |
| | HLKDFPILPGEIFKYK | AAGCAGACCATATAACATCTACCCTCACGGAATCACTGATGTC |
| | WTVTVEDGPTKSDPR | CGTCCTTTGTATTCAAGGAGATTACCAAAAGGTGTAAAACATT |
| | CLTRYYSSFVNMERD | TGAAGGATTTTCCAATTCTGCCAGGAGAAATATTCAAATATAA |
| | LASGLIGPLLICYKES | ATGGACAGTGACTGTAGAAGATGGGCCAACTAAATCAGATCC |
| | VDQRGNQIMSDKRN | TCGGTGCCTGACCCGCTATTACTCTAGTTTCGTTAATATGGAG |
| | VILFSVFDENRSWYL | AGAGATCTAGCTTCAGGACTCATTGGCCCTCTCCTCATCTGCT |
| | TENIQRFLPNPAGVQL | ACAAAGAATCTGTAGATCAAAGAGGAAACCAGATAATGTCAG |
| | EDPEFQASNIMHSING | ACAAGAGGAATGTCATCCTGTTTTCTGTATTTGATGAGAACCG |
| | YVFDSLQLSVCLHEV | AAGCTGGTACCTCACAGAGAATATACAACGCTTTCTCCCCAAT |
| | AYWYILSIGAQTDFLS | CCAGCTGGAGTGCAGCTTGAGGATCCAGAGTTCCAAGCCTCCA |
| | VFFSGYTFKHKMVYE | ACATCATGCACAGCATCAATGGCTATGTTTTTGATAGTTTGCA |
| | DTLTLFPFSGETVFMS | GTTGTCAGTTTGTTTGCATGAGGTGGCATACTGGTACATTCTA |
| | MENPGLWILGCHNSD | AGCATTGGAGCACAGACTGACTTCCTTTCTGTCTTCTTCTCTGG |
| | FRNRGMTALLKVSSC | ATATACCTTCAAACACAAAATGGTCTATGAAGACACACTCACC |
| | DKNTGDYYEDSYEDI | CTATTCCCATTCTCAGGAGAAACTGTCTTCATGTCGATGGAAA |
| | SAYLLSKNNAIEPRSF | ACCCAGGTCTATGGATTCTGGGGTGCCACAACTCAGACTTTCG |
| | SQNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0130 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPGAPGSPA | TATCTCTTCCCACCAACATGATGGCATGGAAGCTTATGTCAAA |
| | GSPTSTEEGTSESATP | GTAGACAGCTGTCCAGAGGAACCCGGCGCGCCAGGTTCTCCTG |
| | ESGPGSEPATSGSETP | CTGGCTCCCCCACCTCAACAGAAGAGGGGACAAGCGAAAGCG |
| | ASSVQLQLRMKNNEE | CTACGCCTGAGAGTGGCCCTGGCTCTGAGCCAGCCACCTCCGG |
| | AEDYDDDLTDSEMD | CTCTGAAACCCCTGCCTCGAGCGTGCAACTTCAACTACGAATG |
| | VVRFDDDNSPSFIQIR | AAAAATAATGAAGAAGCGGAAGACTATGATGATGATCTTACT |
| | SVAKKHPKTWVHYI | GATTCTGAAATGGATGTGGTCAGGTTTGATGATGACAACTCTC |
| | AAEEEDWDYAPLVL | CTTCCTTTATCCAAATTCGCTCAGTTGCCAAGAAGCATCCTAA |
| | APDDRSYKSQYLNNG | AACTTGGGTACATTACATTGCTGCTGAAGAGGAGGACTGGGA |
| | PQRIGRKYKKVRFMA | CTATGCTCCCTTAGTCCTCGCCCCCGATGACAGAAGTTATAAA |
| | YTDETFKTREAIQHES | AGTCAATATTTGAACAATGGCCCTCAGCGGATTGGTAGGAAGT |
| | GILGPLLYGEVGDTL | ACAAAAAAGTCCGATTTATGGCATACACAGATGAAACCTTTAA |
| | LIIFKNQASRPYNIYP | GACTCGTGAAGCTATTCAGCATGAATCAGGAATCTTGGGACCT |
| | HGITDVRPLYSRRLPK | TTACTTTATGGGGAAGTTGGAGACACACTGTTGATTATATTTA |
| | GVKHLKDFPILPGEIF | AGAATCAAGCAAGCAGACCATATAACATCTACCCTCACGGAA |
| | KYKWTVTVEDGPTK | TCACTGATGTCCGTCCTTTGTATTCAAGGAGATTACCAAAAGG |
| | SDPRCLTRYYSSFVN | TGTAAAACATTTGAAGGATTTTCCAATTCTGCCAGGAGAAATA |
| | MERDLASGLIGPLLIC | TTCAAATATAAATGGACAGTGACTGTAGAAGATGGGCCAACT |
| | YKESVDQRGNQIMSD | AAATCAGATCCTCGGTGCCTGACCCGCTATTACTCTAGTTTCGT |
| | KRNVILFSVFDENRS | TAATATGGAGAGAGATCTAGCTTCAGGACTCATTGGCCCTCTC |
| | WYLTENIQRFLPNPA | CTCATCTGCTACAAAGAATCTGTAGATCAAAGAGGAAACCAG |
| | GVQLEDPEFQASNIM | ATAATGTCAGACAAGAGGAATGTCATCCTGTTTTCTGTATTTG |
| | HSINGYVFDSLQLSV | ATGAGAACCGAAGCTGGTACCTCACAGAGAATATACAACGCT |
| | CLHEVAYWYILSIGA | TTCTCCCCAATCCAGCTGGAGTGCAGCTTGAGGATCCAGAGTT |
| | QTDFLSVFFSGYTFK | CCAAGCCTCCAACATCATGCACAGCATCAATGGCTATGTTTTT |
| | HKMVYEDTLTLFPFS | GATAGTTTGCAGTTGTCAGTTTGTTTGCATGAGGTGGCATACT |
| | GETVFMSMENPGLWI | GGTACATTCTAAGCATTGGAGCACAGACTGACTTCCTTTCTGT |
| | LGCHNSDFRNRGMT | CTTCTTCTCTGGATATACCTTCAAACACAAAATGGTCTATGAA |
| | ALLKVSSCDKNTGDY | GACACACTCACCCTATTCCCATTCTCAGGAGAAACTGTCTTCA |
| | YEDSYEDISAYLLSK | TGTCGATGGAAAACCCAGGTCTATGGATTCTGGGGTGCCACAA |
| | NNAIEPRSFSQNPPVL | CTCAGACTTTCGGAACAGAGGCATGACCGCCTTACTGAAGGTT |
| | KRHQREITRTTLQSD | TCTAGTTGTGACAAGAACACTGGTGATTATTACGAGGACAGTT |
| | QEEIDYDDTISVEMK | ATGAAGATATTTCAGCATACTTGCTGAGTAAAAACAATGCCAT |
| | KEDFDIYDEDENQSP | TGAACCAAGAAGCTTCTCTCAAAACCCACCAGTCTTGAAACGC |
| | RSFQKKTRHYFIAAV | CATCAACGGGAAATAACTCGTACTACTCTTCAGTCAGATCAAG |
| | ERLWDYGMSSSPHVL | AGGAAATCGATTATGATGATACCATATCAGTTGAAATGAAGA |
| | RNRAQSGSVPQFKKV | AGGAAGATTTTGACATTTATGATGAGGATGAAAATCAGAGCC |
| | VFQEFTDGSFTQPLY | CCCGCAGCTTTCAAAAGAAAACACGACACTATTTTATTGCTGC |
| | RGELNEHLGLLGPYI | AGTGGAGAGGCTCTGGGATTATGGGATGAGTAGCTCCCCACAT |
| | RAEVEDNIMVTFRNQ | GTTCTAAGAAACAGGGCTCAGAGTGGCAGTGTCCCTCAGTTCA |
| | ASRPYSFYSSLISYEE | AGAAAGTTGTTTTCCAGGAATTTACTGATGGCTCCTTTACTCA |
| | DQRQGAEPRKNFVKP | GCCCTTATACCGTGGAGAACTAAATGAACATTTGGGACTCCTG |
| | NETKTYFWKVQHHM | GGGCCATATATAAGAGCAGAAGTTGAAGATAATATCATGGTA |
| | APTKDEFDCKAWAY | ACTTTCAGAAATCAGGCCTCTCGTCCCTATTCCTTCTATTCTAG |
| | FSDVDLEKDVHSGLI | CCTTATTTCTTATGAGGAAGATCAGAGGCAAGGAGCAGAACCT |
| | GPLLVCHTNTLNPAH | AGAAAAAACTTTGTCAAGCCTAATGAAACCAAAACTTACTTTT |
| | GRQVTVQEFALFFTIF | GGAAAGTGCAACATCATATGGCACCCACTAAAGATGAGTTTG |
| | DETKSWYFTENMER | ACTGCAAAGCCTGGGCTTATTTCTCTGATGTTGACCTGGAAAA |
| | NCRAPCNIQMEDPTF | AGATGTGCACTCAGGCCTGATTGGACCCCTTCTGGTCTGCCAC |
| | KENYRFHAINGYIMD | ACTAACACACTGAACCCTGCTCATGGGAGACAAGTGACAGTA |
| | TLPGLVMAQDQRIR | CAGGAATTTGCTCTGTTTTTCACCATCTTTGATGAGACCAAAA |
| | WYLLSMGSNENIHSI | GCTGGTACTTCACTGAAAATATGGAAAGAAACTGCAGGGCTC |
| | HFSGHVFTVRKKEEY | CCTGCAATATCCAGATGGAAGATCCCACTTTTAAAGAGAATTA |
| | KMALYNLYPGVFET | TCGCTTCCATGCAATCAATGGCTACATAATGGATACACTACCT |
| | VEMLPSKAGIWRVEC | GGCTTAGTAATGGCTCAGGATCAAAGGATTCGATGGTATCTGC |
| | LIGEHLHAGMSTLFL | TCAGCATGGGCAGCAATGAAAACATCCATTCTATTCATTTCAG |
| | VYSNKCQTPLGMAS | TGGACATGTGTTCACTGTACGAAAAAAAGAGGAGTATAAAAT |
| | GHIRDFQITASGQYG | GGCACTGTACAATCTCTATCCAGGTGTTTTTGAGACAGTGGAA |
| | QWAPKLARLHYSGSI | ATGTTACCATCCAAAGCTGGAATTTGGCGGGTGGAATGCCTTA |
| | NAWSTKEPFSWIKVD | TTGGCGAGCATCTACATGCTGGGATGAGCACACTTTTTCTGGT |
| | LLAPMIIHGIKTQGAR | GTACAGCAATAAGTGTCAGACTCCCCTGGGAATGGCTTCTGGA |
| | QKFSSLYISQFIIMYSL | CACATTAGAGATTTTCAGATTACAGCTTCAGGACAATATGGAC |
| | DGKKWQTYRGNSTG | AGTGGGCCCCAAAGCTGGCCAGACTTCATTATTCCGGATCAAT |
| | TLMVFFGNVDSSGIK | CAATGCCTGGAGCACCAAGGAGCCCTTTTCTTGGATCAAGGTG |
| | HNIFNPPIIARYIRLHP | GATCTGTTGGCACCAATGATTATTCACGGCATCAAGACCCAGG |
| | THYSIRSTLRMELMG | GTGCCCGTCAGAAGTTCTCCAGCCTCTACATCTCTCAGTTTATC |
| | CDLNSCSMPLGMESK | ATCATGTATAGTCTTGATGGGAAGAAGTGGCAGACTTATCGAG |
| | AISDAQITASSYFTNM | GAAATTCCACTGGAACCTTAATGGTCTTCTTTGGCAATGTGGA |
| | FATWSPSKARLHLQG | TTCATCTGGGATAAAACACAATATTTTTAACCCTCCAATTATTG |
| | RSNAWRPQVNNPKE | CTCGATACATCCGTTTGCACCCAACTCATTATAGCATTCGCAG |
| | WLQVDFQKTMKVTG | CACTCTTCGCATGGAGTTGATGGGCTGTGATTTAAATAGTTGC |
| | VTTQGVKSLLTSMYV | AGCATGCCATTGGGAATGGAGAGTAAAGCAATATCAGATGCA |
| | KEFLISSSQDGHQWT | CAGATTACTGCTTCATCCTACTTTACCAATATGTTTGCCACCTG |
| | LFFQNGKVKVFQGN | GTCTCCTTCAAAAGCTCGACTTCACCTCCAAGGGAGGAGTAAT |
| | QDSFTPVVNSLDPPLL | GCCTGGAGACCTCAGGTGAATAATCCAAAAGAGTGGCTGCAA |
| | TRYLRIHPQSWVHQI | GTGGACTTCCAGAAGACAATGAAAGTCACAGGAGTAACTACT |
| | ALRMEVLGCEAQDL | CAGGGAGTAAAATCTCTGCTTACCAGCATGTATGTGAAGGAGT |
| | YGAGSPGAETAEQKL | TCCTCATCTCCAGCAGTCAAGATGGCCATCAGTGGACTCTCTT |
| | ISEEDLSPATG | TTTTCAGAATGGCAAAGTAAAGGTTTTTCAGGGAAATCAAGAC |
| | | TCCTTCACACCTGTGGTGAACTCTCTAGACCCACCGTTACTGA |
| | | CTCGCTACCTTCGAATTCACCCCCAGAGTTGGGTGCACCAGAT |
| | | TGCCCTGAGGATGGAGGTTCTGGGCTGCGAGGCACAGGACCT |
| | | CTACGGCGCCGGATCACCTGGGGCCGAAACGGCCGAACAAAA |
| | | ACTCATCTCAGAAGAGGATCTGTCACCTGCAACCGGTTGA |
| pBC0131 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVASVAGAPGSPAG | AAATGGATGTGGTCAGGTTTGATGATGACAACTCTCCTTCCTT |
| | SPTSTEEGTSESATPE | TATCCAAATTCGCTCAGTTGCCTCTGTAGCAGGCGCGCCAGGT |
| | SGPGSEPATSGSETPA | TCTCCTGCTGGCTCCCCCACCTCAACAGAAGAGGGGACAAGCG |
| | SSKKHPKTWVHYIAA | AAAGCGCTACGCCTGAGAGTGGCCCTGGCTCTGAGCCAGCCA |
| | EEEDWDYAPLVLAPD | CCTCCGGCTCTGAAACCCCTGCCTCGAGCAAGAAGCATCCTAA |
| | DRSYKSQYLNNGPQR | AACTTGGGTACATTACATTGCTGCTGAAGAGGAGGACTGGGA |
| | IGRKYKKVRFMAYT | CTATGCTCCCTTAGTCCTCGCCCCCGATGACAGAAGTTATAAA |
| | DETFKTREAIQHESGI | AGTCAATATTTGAACAATGGCCCTCAGCGGATTGGTAGGAAGT |
| | LGPLLYGEVGDTLLII | ACAAAAAAGTCCGATTTATGGCATACACAGATGAAACCTTTAA |
| | FKNQASRPYNIYPHGI | GACTCGTGAAGCTATTCAGCATGAATCAGGAATCTTGGGACCT |
| | TDVRPLYSRRLPKGV | TTACTTTATGGGGAAGTTGGAGACACACTGTTGATTATATTTA |
| | KHLKDFPILPGEIFKY | AGAATCAAGCAAGCAGACCATATAACATCTACCCTCACGGAA |
| | KWTVTVEDGPTKSDP | TCACTGATGTCCGTCCTTTGTATTCAAGGAGATTACCAAAAGG |
| | RCLTRYYSSFVNMER | TGTAAAACATTTGAAGGATTTTCCAATTCTGCCAGGAGAAATA |
| | DLASGLIGPLLICYKE | TTCAAATATAAATGGACAGTGACTGTAGAAGATGGGCCAACT |
| | SVDQRGNQIMSDKRN | AAATCAGATCCTCGGTGCCTGACCCGCTATTACTCTAGTTTCGT |
| | VILFSVFDENRSWYL | TAATATGGAGAGAGATCTAGCTTCAGGACTCATTGGCCCTCTC |
| | TENIQRFLPNPAGVQL | CTCATCTGCTACAAAGAATCTGTAGATCAAAGAGGAAACCAG |
| | EDPEFQASNIMHSING | ATAATGTCAGACAAGAGGAATGTCATCCTGTTTTCTGTATTTG |
| | YVFDSLQLSVCLHEV | ATGAGAACCGAAGCTGGTACCTCACAGAGAATATACAACGCT |
| | AYWYILSIGAQTDFLS | TTCTCCCCAATCCAGCTGGAGTGCAGCTTGAGGATCCAGAGTT |
| | VFFSGYTFKHKMVYE | CCAAGCCTCCAACATCATGCACAGCATCAATGGCTATGTTTTT |
| | DTLTLFPFSGETVFMS | GATAGTTTGCAGTTGTCAGTTTGTTTGCATGAGGTGGCATACT |
| | MENPGLWILGCHNSD | GGTACATTCTAAGCATTGGAGCACAGACTGACTTCCTTTCTGT |
| | FRNRGMTALLKVSSC | CTTCTTCTCTGGATATACCTTCAAACACAAAATGGTCTATGAA |
| | DKNTGDYYEDSYEDI | GACACACTCACCCTATTCCCATTCTCAGGAGAAACTGTCTTCA |
| | SAYLLSKNNAIEPRSF | TGTCGATGGAAAACCCAGGTCTATGGATTCTGGGGTGCCACAA |
| | SQNPPVLKRHQREITR | CTCAGACTTTCGGAACAGAGGCATGACCGCCTTACTGAAGGTT |
| | TTLQSDQEEIDYDDTI | TCTAGTTGTGACAAGAACACTGGTGATTATTACGAGGACAGTT |
| | SVEMKKEDFDIYDED | ATGAAGATATTTCAGCATACTTGCTGAGTAAAAACAATGCCAT |
| | ENQSPRSFQKKTRHY | TGAACCAAGAAGCTTCTCTCAAAACCCACCAGTCTTGAAACGC |
| | FIAAVERLWDYGMSS | CATCAACGGGAAATAACTCGTACTACTCTTCAGTCAGATCAAG |
| | SPHVLRNRAQSGSVP | AGGAAATCGATTATGATGATACCATATCAGTTGAAATGAAGA |
| | QFKKVVFQEFTDGSF | AGGAAGATTTTGACATTTATGATGAGGATGAAAATCAGAGCC |
| | TQPLYRGELNEHLGL | CCCGCAGCTTTCAAAAGAAAACACGACACTATTTTATTGCTGC |
| | LGPYIRAEVEDNIMV | AGTGGAGAGGCTCTGGGATTATGGGATGAGTAGCTCCCCACAT |
| | TFRNQASRPYSFYSSL | GTTCTAAGAAACAGGGCTCAGAGTGGCAGTGTCCCTCAGTTCA |
| | ISYEEDQRQGAEPRK | AGAAAGTTGTTTTCCAGGAATTTACTGATGGCTCCTTTACTCA |
| | NFVKPNETKTYFWK | GCCCTTATACCGTGGAGAACTAAATGAACATTTGGGACTCCTG |
| | VQHHMAPTKDEFDC | GGGCCATATATAAGAGCAGAAGTTGAAGATAATATCATGGTA |
| | KAWAYFSDVDLEKD | ACTTTCAGAAATCAGGCCTCTCGTCCCTATTCCTTCTATTCTAG |
| | VHSGLIGPLLVCHTN | CCTTATTTCTTATGAGGAAGATCAGAGGCAAGGAGCAGAACCT |
| | TLNPAHGRQVTVQEF | AGAAAAAACTTTGTCAAGCCTAATGAAACCAAAACTTACTTTT |
| | ALFFTIFDETKSWYFT | GGAAAGTGCAACATCATATGGCACCCACTAAAGATGAGTTTG |
| | ENMERNCRAPCNIQM | ACTGCAAAGCCTGGGCTTATTTCTCTGATGTTGACCTGGAAAA |
| | EDPTFKENYRFHAIN | AGATGTGCACTCAGGCCTGATTGGACCCCTTCTGGTCTGCCAC |
| | GYIMDTLPGLVMAQ | ACTAACACACTGAACCCTGCTCATGGGAGACAAGTGACAGTA |
| | DQRIRWYLLSMGSNE | CAGGAATTTGCTCTGTTTTTCACCATCTTTGATGAGACCAAAA |
| | NIHSIHFSGHVFTVRK | GCTGGTACTTCACTGAAAATATGGAAAGAAACTGCAGGGCTC |
| | KEEYKMALYNLYPG | CCTGCAATATCCAGATGGAAGATCCCACTTTTAAAGAGAATTA |
| | VFETVEMLPSKAGIW | TCGCTTCCATGCAATCAATGGCTACATAATGGATACACTACCT |
| | RVECLIGEHLHAGMS | GGCTTAGTAATGGCTCAGGATCAAAGGATTCGATGGTATCTGC |
| | TLFLVYSNKCQTPLG | TCAGCATGGGCAGCAATGAAAACATCCATTCTATTCATTTCAG |
| | MASGHIRDFQITASG | TGGACATGTGTTCACTGTACGAAAAAAAGAGGAGTATAAAAT |
| | QYGQWAPKLARLHY | GGCACTGTACAATCTCTATCCAGGTGTTTTTGAGACAGTGGAA |
| | SGSINAWSTKEPFSWI | ATGTTACCATCCAAAGCTGGAATTTGGCGGGTGGAATGCCTTA |
| | KVDLLAPMIIHGIKTQ | TTGGCGAGCATCTACATGCTGGGATGAGCACACTTTTTCTGGT |
| | GARQKFSSLYISQFII | GTACAGCAATAAGTGTCAGACTCCCCTGGGAATGGCTTCTGGA |
| | MYSLDGKKWQTYRG | CACATTAGAGATTTTCAGATTACAGCTTCAGGACAATATGGAC |
| | NSTGTLMVFFGNVDS | AGTGGGCCCCAAAGCTGGCCAGACTTCATTATTCCGGATCAAT |
| | SGIKHNIFNPPIIARYI | CAATGCCTGGAGCACCAAGGAGCCCTTTTCTTGGATCAAGGTG |
| | RLHPTHYSIRSTLRME | GATCTGTTGGCACCAATGATTATTCACGGCATCAAGACCCAGG |
| | LMGCDLNSCSMPLG | GTGCCCGTCAGAAGTTCTCCAGCCTCTACATCTCTCAGTTTATC |
| | MESKAISDAQITASSY | ATCATGTATAGTCTTGATGGGAAGAAGTGGCAGACTTATCGAG |
| | FTNMFATWSPSKARL | GAAATTCCACTGGAACCTTAATGGTCTTCTTTGGCAATGTGGA |
| | HLQGRSNAWRPQVN | TTCATCTGGGATAAAACACAATATTTTTAACCCTCCAATTATTG |
| | NPKEWLQVDFQKTM | CTCGATACATCCGTTTGCACCCAACTCATTATAGCATTCGCAG |
| | KVTGVTTQGVKSLLT | CACTCTTCGCATGGAGTTGATGGGCTGTGATTTAAATAGTTGC |
| | SMYVKEFLISSSQDG | AGCATGCCATTGGGAATGGAGAGTAAAGCAATATCAGATGCA |
| | HQWTLFFQNGKVKV | CAGATTACTGCTTCATCCTACTTTACCAATATGTTTGCCACCTG |
| | FQGNQDSFTPVVNSL | GTCTCCTTCAAAAGCTCGACTTCACCTCCAAGGGAGGAGTAAT |
| | DPPLLTRYLRIHPQSW | GCCTGGAGACCTCAGGTGAATAATCCAAAAGAGTGGCTGCAA |
| | VHQIALRMEVLGCEA | GTGGACTTCCAGAAGACAATGAAAGTCACAGGAGTAACTACT |
| | QDLYGAGSPGAETAE | CAGGGAGTAAAATCTCTGCTTACCAGCATGTATGTGAAGGAGT |
| | QKLISEEDLSPATG | TCCTCATCTCCAGCAGTCAAGATGGCCATCAGTGGACTCTCTT |
| | | TTTTCAGAATGGCAAAGTAAAGGTTTTTCAGGGAAATCAAGAC |
| | | TCCTTCACACCTGTGGTGAACTCTCTAGACCCACCGTTACTGA |
| | | CTCGCTACCTTCGAATTCACCCCCAGAGTTGGGTGCACCAGAT |
| | | TGCCCTGAGGATGGAGGTTCTGGGCTGCGAGGCACAGGACCT |
| | | CTACGGCGCCGGATCACCTGGGGCCGAAACGGCCGAACAAAA |
| | | ACTCATCTCAGAAGAGGATCTGTCACCTGCAACCGGTTGA |
| pBC0132 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDGAPGSPAGSPTST | GTACATTACATTGCTGCTGAAGAGGAGGACTGGGACTATGCTC |
| | EEGTSESATPESGPGS | CCTTAGTCCTCGCCCCCGATGGCGCGCCAGGTTCTCCTGCTGG |
| | EPATSGSETPASSDRS | CTCCCCCACCTCAACAGAAGAGGGGACAAGCGAAAGCGCTAC |
| | YKSQYLNNGPQRIGR | GCCTGAGAGTGGCCCTGGCTCTGAGCCAGCCACCTCCGGCTCT |
| | KYKKVRFMAYTDET | GAAACCCCTGCCTCGAGCGACAGAAGTTATAAAAGTCAATATT |
| | FKTREAIQHESGILGP | TGAACAATGGCCCTCAGCGGATTGGTAGGAAGTACAAAAAAG |
| | LLYGEVGDTLLIIFKN | TCCGATTTATGGCATACACAGATGAAACCTTTAAGACTCGTGA |
| | QASRPYNIYPHGITDV | AGCTATTCAGCATGAATCAGGAATCTTGGGACCTTTACTTTAT |
| | RPLYSRRLPKGVKHL | GGGGAAGTTGGAGACACACTGTTGATTATATTTAAGAATCAAG |
| | KDFPILPGEIFKYKWT | CAAGCAGACCATATAACATCTACCCTCACGGAATCACTGATGT |
| | VTVEDGPTKSDPRCL | CCGTCCTTTGTATTCAAGGAGATTACCAAAAGGTGTAAAACAT |
| | TRYYSSFVNMERDLA | TTGAAGGATTTTCCAATTCTGCCAGGAGAAATATTCAAATATA |
| | SGLIGPLLICYKESVD | AATGGACAGTGACTGTAGAAGATGGGCCAACTAAATCAGATC |
| | QRGNQIMSDKRNVIL | CTCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0170 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIGAP | TTTGAACAATGGCCCTCAGCGGATTGGTAGGAAGTACAAAAA |
| | GSPAGSPTSTEEGTSE | AGTCCGATTTATGGCATACACAGATGAAACCTTTAAGACTCGT |
| | SATPESGPGSEPATSG | GAAGCTATTGGCGCGCCAGGTTCTCCTGCTGGCTCCCCCACCT |
| | SETPASSQHESGILGP | CAACAGAAGAGGGGACAAGCGAAAGCGCTACGCCTGAGAGTG |
| | LLYGEVGDTLLIIFKN | GCCCTGGCTCTGAGCCAGCCACCTCCGGCTCTGAAACCCCTGC |
| | QASRPYNIYPHGITDV | CTCGAGCCAGCATGAATCAGGAATCTTGGGACCTTTACTTTAT |
| | RPLYSRRLPKGVKHL | GGGGAAGTTGGAGACACACTGTTGATTATATTTAAGAATCAAG |
| | KDFPILPGEIFKYKWT | CAAGCAGACCATATAACATCTACCCTCACGGAATCACTGATGT |
| | VTVEDGPTKSDPRCL | CCGTCCTTTGTATTCAAGGAGATTACCAAAAGGTGTAAAACAT |
| | TRYYSSFVNMERDLA | TTGAAGGATTTTCCAATTCTGCCAGGAGAAATATTCAAATATA |
| | SGLIGPLLICYKESVD | AATGGACAGTGACTGTAGAAGATGGGCCAACTAAATCAGATC |
| | QRGNQIMSDKRNVIL | CTCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0133 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GAPGSPAGSPTSTEEG | AGCAAGCAGACCATATAACATCTACCCTCACGGAATCACTGAT |
| | TSESATPESGPGSEPA | GTCCGTCCTTTGTATTCAAGGAGACTCCCTAAAGGCGCGCCAG |
| | TSGSETPASSLPKGVK | GTTCTCCTGCTGGCTCCCCCACCTCAACAGAAGAGGGGACAAG |
| | HLKDFPILPGEIFKYK | CGAAAGCGCTACGCCTGAGAGTGGCCCTGGCTCTGAGCCAGC |
| | WTVTVEDGPTKSDPR | CACCTCCGGCTCTGAAACCCCTGCCTCGAGCTTACCAAAAGGT |
| | CLTRYYSSFVNMERD | GTAAAACATTTGAAGGATTTTCCAATTCTGCCAGGAGAAATAT |
| | LASGLIGPLLICYKES | TCAAATATAAATGGACAGTGACTGTAGAAGATGGGCCAACTA |
| | VDQRGNQIMSDKRN | AATCAGATCCTCGGTGCCTGACCCGCTATTACTCTAGTTTCGTT |
| | VILFSVFDENRSWYL | AATATGGAGAGAGATCTAGCTTCAGGACTCATTGGCCCTCTCC |
| | TENIQRFLPNPAGVQL | TCATCTGCTACAAAGAATCTGTAGATCAAAGAGGAAACCAGA |
| | EDPEFQASNIMHSING | TAATGTCAGACAAGAGGAATGTCATCCTGTTTTCTGTATTTGA |
| | YVFDSLQLSVCLHEV | TGAGAACCGAAGCTGGTACCTCACAGAGAATATACAACGCTTT |
| | AYWYILSIGAQTDFLS | CTCCCCAATCCAGCTGGAGTGCAGCTTGAGGATCCAGAGTTCC |
| | VFFSGYTFKHKMVYE | AAGCCTCCAACATCATGCACAGCATCAATGGCTATGTTTTTGA |
| | DTLTLFPFSGETVFMS | TAGTTTGCAGTTGTCAGTTTGTTTGCATGAGGTGGCATACTGGT |
| | MENPGLWILGCHNSD | ACATTCTAAGCATTGGAGCACAGACTGACTTCCTTTCTGTCTTC |
| | FRNRGMTALLKVSSC | TTCTCTGGATATACCTTCAAACACAAAATGGTCTATGAAGACA |
| | DKNTGDYYEDSYEDI | CACTCACCCTATTCCCATTCTCAGGAGAAACTGTCTTCATGTCG |
| | SAYLLSKNNAIEPRSF | ATGGAAAACCCAGGTCTATGGATTCTGGGGTGCCACAACTCAG |
| | SQNPPVLKRHQREITR | ACTTTCGGAACAGAGGCATGACCGCCTTACTGAAGGTTTCTAG |
| | TTLQSDQEEIDYDDTI | TTGTGACAAGAACACTGGTGATTATTACGAGGACAGTTATGAA |
| | SVEMKKEDFDIYDED | GATATTTCAGCATACTTGCTGAGTAAAAACAATGCCATTGAAC |
| | ENQSPRSFQKKTRHY | CAAGAAGCTTCTCTCAAAACCCACCAGTCTTGAAACGCCATCA |
| | FIAAVERLWDYGMSS | ACGGGAAATAACTCGTACTACTCTTCAGTCAGATCAAGAGGA |
| | SPHVLRNRAQSGSVP | AATCGATTATGATGATACCATATCAGTTGAAATGAAGAAGGA |
| | QFKKVVFQEFTDGSF | AGATTTTGACATTTATGATGAGGATGAAAATCAGAGCCCCCGC |
| | TQPLYRGELNEHLGL | AGCTTTCAAAAGAAAACACGACACTATTTTATTGCTGCAGTGG |
| | LGPYIRAEVEDNIMV | AGAGGCTCTGGGATTATGGGATGAGTAGCTCCCCACATGTTCT |
| | TFRNQASRPYSFYSSL | AAGAAACAGGGCTCAGAGTGGCAGTGTCCCTCAGTTCAAGAA |
| | ISYEEDQRQGAEPRK | AGTTGTTTTCCAGGAATTTACTGATGGCTCCTTTACTCAGCCCT |
| | NFVKPNETKTYFWK | TATACCGTGGAGAACTAAATGAACATTTGGGACTCCTGGGGCC |
| | VQHHMAPTKDEFDC | ATATATAAGAGCAGAAGTTGAAGATAATATCATGGTAACTTTC |
| | KAWAYFSDVDLEKD | AGAAATCAGGCCTCTCGTCCCTATTCCTTCTATTCTAGCCTTAT |
| | VHSGLIGPLLVCHTN | TTCTTATGAGGAAGATCAGAGGCAAGGAGCAGAACCTAGAAA |
| | TLNPAHGRQVTVQEF | AAACTTTGTCAAGCCTAATGAAACCAAAACTTACTTTTGGAAA |
| | ALFFTIFDETKSWYFT | GTGCAACATCATATGGCACCCACTAAAGATGAGTTTGACTGCA |
| | ENMERNCRAPCNIQM | AAGCCTGGGCTTATTTCTCTGATGTTGACCTGGAAAAAGATGT |
| | EDPTFKENYRFHAIN | GCACTCAGGCCTGATTGGACCCCTTCTGGTCTGCCACACTAAC |
| | GYIMDTLPGLVMAQ | ACACTGAACCCTGCTCATGGGAGACAAGTGACAGTACAGGAA |
| | DQRIRWYLLSMGSNE | TTTGCTCTGTTTTTCACCATCTTTGATGAGACCAAAAGCTGGTA |
| | NIHSIHFSGHVFTVRK | CTTCACTGAAAATATGGAAAGAAACTGCAGGGCTCCCTGCAAT |
| | KEEYKMALYNLYPG | ATCCAGATGGAAGATCCCACTTTTAAAGAGAATTATCGCTTCC |
| | VFETVEMLPSKAGIW | ATGCAATCAATGGCTACATAATGGATACACTACCTGGCTTAGT |
| | RVECLIGEHLHAGMS | AATGGCTCAGGATCAAAGGATTCGATGGTATCTGCTCAGCATG |
| | TLFLVYSNKCQTPLG | GGCAGCAATGAAAACATCCATTCTATTCATTTCAGTGGACATG |
| | MASGHIRDFQITASG | TGTTCACTGTACGAAAAAAAGAGGAGTATAAAATGGCACTGT |
| | QYGQWAPKLARLHY | ACAATCTCTATCCAGGTGTTTTTGAGACAGTGGAAATGTTACC |
| | SGSINAWSTKEPFSWI | ATCCAAAGCTGGAATTTGGCGGGTGGAATGCCTTATTGGCGAG |
| | KVDLLAPMIIHGIKTQ | CATCTACATGCTGGGATGAGCACACTTTTTCTGGTGTACAGCA |
| | GARQKFSSLYISQFII | ATAAGTGTCAGACTCCCCTGGGAATGGCTTCTGGACACATTAG |
| | MYSLDGKKWQTYRG | AGATTTTCAGATTACAGCTTCAGGACAATATGGACAGTGGGCC |
| | NSTGTLMVFFGNVDS | CCAAAGCTGGCCAGACTTCATTATTCCGGATCAATCAATGCCT |
| | SGIKHNIFNPPIIARYI | GGAGCACCAAGGAGCCCTTTTCTTGGATCAAGGTGGATCTGTT |
| | RLHPTHYSIRSTLRME | GGCACCAATGATTATTCACGGCATCAAGACCCAGGGTGCCCGT |
| | LMGCDLNSCSMPLG | CAGAAGTTCTCCAGCCTCTACATCTCTCAGTTTATCATCATGTA |
| | MESKAISDAQITASSY | TAGTCTTGATGGGAAGAAGTGGCAGACTTATCGAGGAAATTCC |
| | FTNMFATWSPSKARL | ACTGGAACCTTAATGGTCTTCTTTGGCAATGTGGATTCATCTG |
| | HLQGRSNAWRPQVN | GGATAAAACACAATATTTTTAACCCTCCAATTATTGCTCGATA |
| | NPKEWLQVDFQKTM | CATCCGTTTGCACCCAACTCATTATAGCATTCGCAGCACTCTTC |
| | KVTGVTTQGVKSLLT | GCATGGAGTTGATGGGCTGTGATTTAAATAGTTGCAGCATGCC |
| | SMYVKEFLISSSQDG | ATTGGGAATGGAGAGTAAAGCAATATCAGATGCACAGATTAC |
| | HQWTLFFQNGKVKV | TGCTTCATCCTACTTTACCAATATGTTTGCCACCTGGTCTCCTT |
| | FQGNQDSFTPVVNSL | CAAAAGCTCGACTTCACCTCCAAGGGAGGAGTAATGCCTGGA |
| | DPPLLTRYLRIHPQSW | GACCTCAGGTGAATAATCCAAAAGAGTGGCTGCAAGTGGACT |
| | VHQIALRMEVLGCEA | TCCAGAAGACAATGAAAGTCACAGGAGTAACTACTCAGGGAG |
| | QDLYGAGSPGAETAE | TAAAATCTCTGCTTACCAGCATGTATGTGAAGGAGTTCCTCAT |
| | QKLISEEDLSPATG | CTCCAGCAGTCAAGATGGCCATCAGTGGACTCTCTTTTTTCAG |
| | | AATGGCAAAGTAAAGGTTTTTCAGGGAAATCAAGACTCCTTCA |
| | | CACCTGTGGTGAACTCTCTAGACCCACCGTTACTGACTCGCTA |
| | | CCTTCGAATTCACCCCCAGAGTTGGGTGCACCAGATTGCCCTG |
| | | AGGATGGAGGTTCTGGGCTGCGAGGCACAGGACCTCTACGGC |
| | | GCCGGATCACCTGGGGCCGAAACGGCCGAACAAAAACTCATC |
| | | TCAGAAGAGGATCTGTCACCTGCAACCGGTTGA |
| pBC0171 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEGAPGS | GTCCGTCCTTTGTATTCAAGGAGATTACCAAAAGGTGTAAAAC |
| | PAGSPTSTEEGTSESA | ATTTGAAGGATTTTCCAATTCTGCCAGGAGAAATATTCAAATA |
| | TPESGPGSEPATSGSE | TAAATGGACAGTGACTGTAGAAGGCGCGCCAGGTTCTCCTGCT |
| | TPASSDGPTKSDPRCL | GGCTCCCCCACCTCAACAGAAGAGGGGACAAGCGAAAGCGCT |
| | TRYYSSFVNMERDLA | ACGCCTGAGAGTGGCCCTGGCTCTGAGCCAGCCACCTCCGGCT |
| | SGLIGPLLICYKESVD | CTGAAACCCCTGCCTCGAGCGATGGGCCAACTAAATCAGATCC |
| | QRGNQIMSDKRNVIL | TCGGTGCCTGACCCGCTATTACTCTAGTTTCGTTAATATGGAG |
| | FSVFDENRSWYLTEN | AGAGATCTAGCTTCAGGACTCATTGGCCCTCTCCTCATCTGCT |
| | IQRFLPNPAGVQLEDP | ACAAAGAATCTGTAGATCAAAGAGGAAACCAGATAATGTCAG |
| | EFQASNIMHSINGYVF | ACAAGAGGAATGTCATCCTGTTTTCTGTATTTGATGAGAACCG |
| | DSLQLSVCLHEVAY | AAGCTGGTACCTCACAGAGAATATACAACGCTTTCTCCCCAAT |
| | WYILSIGAQTDFLSVF | CCAGCTGGAGTGCAGCTTGAGGATCCAGAGTTCCAAGCCTCCA |
| | FSGYTFKHKMVYEDT | ACATCATGCACAGCATCAATGGCTATGTTTTTGATAGTTTGCA |
| | LTLFPFSGETVFMSM | GTTGTCAGTTTGTTTGCATGAGGTGGCATACTGGTACATTCTA |
| | ENPGLWILGCHNSDF | AGCATTGGAGCACAGACTGACTTCCTTTCTGTCTTCTTCTCTGG |
| | RNRGMTALLKVSSCD | ATATACCTTCAAACACAAAATGGTCTATGAAGACACACTCACC |
| | KNTGDYYEDSYEDIS | CTATTCCCATTCTCAGGAGAAACTGTCTTCATGTCGATGGAAA |
| | AYLLSKNNAIEPRSFS | ACCCAGGTCTATGGATTCTGGGGTGCCACAACTCAGACTTTCG |
| | QNPPVLKRHQREITR | GAACAGAGGCATGACCGCCTTACTGAAGGTTTCTAGTTGTGAC |
| | TTLQSDQEEIDYDDTI | AAGAACACTGGTGATTATTACGAGGACAGTTATGAAGATATTT |
| | SVEMKKEDFDIYDED | CAGCATACTTGCTGAGTAAAAACAATGCCATTGAACCAAGAA |
| | ENQSPRSFQKKTRHY | GCTTCTCTCAAAACCCACCAGTCTTGAAACGCCATCAACGGGA |
| | FIAAVERLWDYGMSS | AATAACTCGTACTACTCTTCAGTCAGATCAAGAGGAAATCGAT |
| | SPHVLRNRAQSGSVP | TATGATGATACCATATCAGTTGAAATGAAGAAGGAAGATTTTG |
| | QFKKVVFQEFTDGSF | ACATTTATGATGAGGATGAAAATCAGAGCCCCCGCAGCTTTCA |
| | TQPLYRGELNEHLGL | AAAGAAAACACGACACTATTTTATTGCTGCAGTGGAGAGGCTC |
| | LGPYIRAEVEDNIMV | TGGGATTATGGGATGAGTAGCTCCCCACATGTTCTAAGAAACA |
| | TFRNQASRPYSFYSSL | GGGCTCAGAGTGGCAGTGTCCCTCAGTTCAAGAAAGTTGTTTT |
| | ISYEEDQRQGAEPRK | CCAGGAATTTACTGATGGCTCCTTTACTCAGCCCTTATACCGTG |
| | NFVKPNETKTYFWK | GAGAACTAAATGAACATTTGGGACTCCTGGGGCCATATATAA |
| | VQHHMAPTKDEFDC | GAGCAGAAGTTGAAGATAATATCATGGTAACTTTCAGAAATC |
| | KAWAYFSDVDLEKD | AGGCCTCTCGTCCCTATTCCTTCTATTCTAGCCTTATTTCTTATG |
| | VHSGLIGPLLVCHTN | AGGAAGATCAGAGGCAAGGAGCAGAACCTAGAAAAAACTTTG |
| | TLNPAHGRQVTVQEF | TCAAGCCTAATGAAACCAAAACTTACTTTTGGAAAGTGCAACA |
| | ALFFTIFDETKSWYFT | TCATATGGCACCCACTAAAGATGAGTTTGACTGCAAAGCCTGG |
| | ENMERNCRAPCNIQM | GCTTATTTCTCTGATGTTGACCTGGAAAAAGATGTGCACTCAG |
| | EDPTFKENYRFHAIN | GCCTGATTGGACCCCTTCTGGTCTGCCACACTAACACACTGAA |
| | GYIMDTLPGLVMAQ | CCCTGCTCATGGGAGACAAGTGACAGTACAGGAATTTGCTCTG |
| | DQRIRWYLLSMGSNE | TTTTTCACCATCTTTGATGAGACCAAAAGCTGGTACTTCACTG |
| | NIHSIHFSGHVFTVRK | AAAATATGGAAAGAAACTGCAGGGCTCCCTGCAATATCCAGA |
| | KEEYKMALYNLYPG | TGGAAGATCCCACTTTTAAAGAGAATTATCGCTTCCATGCAAT |
| | VFETVEMLPSKAGIW | CAATGGCTACATAATGGATACACTACCTGGCTTAGTAATGGCT |
| | RVECLIGEHLHAGMS | CAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0134 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GAPGSPAGSPTSTEEG | AGACAAGAGGAATGTCATCCTGTTTTCTGTATTTGATGAGAAC |
| | TSESATPESGPGSEPA | CGAAGCTGGTACCTCACAGAGAATATACAACGCTTTCTCCCCA |
| | TSGSETPASSGVQLED | ATCCAGCTGGCGCGCCAGGTTCTCCTGCTGGCTCCCCCACCTC |
| | PEFQASNIMHSINGYV | AACAGAAGAGGGGACAAGCGAAAGCGCTACGCCTGAGAGTG |
| | FDSLQLSVCLHEVAY | GCCCTGGCTCTGAGCCAGCCACCTCCGGCTCTGAAACCCCTGC |
| | WYILSIGAQTDFLSVF | CTCGAGCGGAGTGCAGCTTGAGGATCCAGAGTTCCAAGCCTCC |
| | FSGYTFKHKMVYEDT | AACATCATGCACAGCATCAATGGCTATGTTTTTGATAGTTTGC |
| | LTLFPFSGETVFMSM | AGTTGTCAGTTTGTTTGCATGAGGTGGCATACTGGTACATTCT |
| | ENPGLWILGCHNSDF | AAGCATTGGAGCACAGACTGACTTCCTTTCTGTCTTCTTCTCTG |
| | RNRGMTALLKVSSCD | GATATACCTTCAAACACAAAATGGTCTATGAAGACACACTCAC |
| | KNTGDYYEDSYEDIS | CCTATTCCCATTCTCAGGAGAAACTGTCTTCATGTCGATGGAA |
| | AYLLSKNNAIEPRSFS | AACCCAGGTCTATGGATTCTGGGGTGCCACAACTCAGACTTTC |
| | QNPPVLKRHQREITR | GGAACAGAGGCATGACCGCCTTACTGAAGGTTTCTAGTTGTGA |
| | TTLQSDQEEIDYDDTI | CAAGAACACTGGTGATTATTACGAGGACAGTTATGAAGATATT |
| | SVEMKKEDFDIYDED | TCAGCATACTTGCTGAGTAAAAACAATGCCATTGAACCAAGA |
| | ENQSPRSFQKKTRHY | AGCTTCTCTCAAAACCCACCAGTCTTGAAACGCCATCAACGGG |
| | FIAAVERLWDYGMSS | AAATAACTCGTACTACTCTTCAGTCAGATCAAGAGGAAATCGA |
| | SPHVLRNRAQSGSVP | TTATGATGATACCATATCAGTTGAAATGAAGAAGGAAGATTTT |
| | QFKKVVFQEFTDGSF | GACATTTATGATGAGGATGAAAATCAGAGCCCCCGCAGCTTTC |
| | TQPLYRGELNEHLGL | AAAAGAAAACACGACACTATTTTATTGCTGCAGTGGAGAGGC |
| | LGPYIRAEVEDNIMV | TCTGGGATTATGGGATGAGTAGCTCCCCACATGTTCTAAGAAA |
| | TFRNQASRPYSFYSSL | CAGGGCTCAGAGTGGCAGTGTCCCTCAGTTCAAGAAAGTTGTT |
| | ISYEEDQRQGAEPRK | TTCCAGGAATTTACTGATGGCTCCTTTACTCAGCCCTTATACCG |
| | NFVKPNETKTYFWK | TGGAGAACTAAATGAACATTTGGGACTCCTGGGGCCATATATA |
| | VQHHMAPTKDEFDC | AGAGCAGAAGTTGAAGATAATATCATGGTAACTTTCAGAAAT |
| | KAWAYFSDVDLEKD | CAGGCCTCTCGTCCCTATTCCTTCTATTCTAGCCTTATTTCTTAT |
| | VHSGLIGPLLVCHTN | GAGGAAGATCAGAGGCAAGGAGCAGAACCTAGAAAAAACTTT |
| | TLNPAHGRQVTVQEF | GTCAAGCCTAATGAAACCAAAACTTACTTTTGGAAAGTGCAAC |
| | ALFFTIFDETKSWYFT | ATCATATGGCACCCACTAAAGATGAGTTTGACTGCAAAGCCTG |
| | ENMERNCRAPCNIQM | GGCTTATTTCTCTGATGTTGACCTGGAAAAAGATGTGCACTCA |
| | EDPTFKENYRFHAIN | GGCCTGATTGGACCCCTTCTGGTCTGCCACACTAACACACTGA |
| | GYIMDTLPGLVMAQ | ACCCTGCTCATGGGAGACAAGTGACAGTACAGGAATTTGCTCT |
| | DQRIRWYLLSMGSNE | GTTTTTCACCATCTTTGATGAGACCAAAAGCTGGTACTTCACT |
| | NIHSIHFSGHVFTVRK | GAAAATATGGAAAGAAACTGCAGGGCTCCCTGCAATATCCAG |
| | KEEYKMALYNLYPG | ATGGAAGATCCCACTTTTAAAGAGAATTATCGCTTCCATGCAA |
| | VFETVEMLPSKAGIW | TCAATGGCTACATAATGGATACACTACCTGGCTTAGTAATGGC |
| | RVECLIGEHLHAGMS | TCAGGATCAAAGGATTCGATGGTATCTGCTCAGCATGGGCAGC |
| | TLFLVYSNKCQTPLG | AATGAAAACATCCATTCTATTCATTTCAGTGGACATGTGTTCA |
| | MASGHIRDFQITASG | CTGTACGAAAAAAAGAGGAGTATAAAATGGCACTGTACAATC |
| | QYGQWAPKLARLHY | TCTATCCAGGTGTTTTTGAGACAGTGGAAATGTTACCATCCAA |
| | SGSINAWSTKEPFSWI | AGCTGGAATTTGGCGGGTGGAATGCCTTATTGGCGAGCATCTA |
| | KVDLLAPMIIHGIKTQ | CATGCTGGGATGAGCACACTTTTTCTGGTGTACAGCAATAAGT |
| | GARQKFSSLYISQFII | GTCAGACTCCCCTGGGAATGGCTTCTGGACACATTAGAGATTT |
| | MYSLDGKKWQTYRG | TCAGATTACAGCTTCAGGACAATATGGACAGTGGGCCCCAAA |
| | NSTGTLMVFFGNVDS | GCTGGCCAGACTTCATTATTCCGGATCAATCAATGCCTGGAGC |
| | SGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0172 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKGAPGS | CCTATTCCCATTCTCAGGAGAAACTGTCTTCATGTCGATGGAA |
| | PAGSPTSTEEGTSESA | AACCCAGGTCTATGGATTCTGGGGTGCCACAACTCAGACTTTC |
| | TPESGPGSEPATSGSE | GGAACAGAGGCATGACCGCCTTACTGAAGGTTTCTAGTTGTGA |
| | TPASSNTGDYYEDSY | CAAGGGCGCGCCAGGTTCTCCTGCTGGCTCCCCCACCTCAACA |
| | EDISAYLLSKNNAIEP | GAAGAGGGGACAAGCGAAAGCGCTACGCCTGAGAGTGGCCCT |
| | RSFSQNPPVLKRHQR | GGCTCTGAGCCAGCCACCTCCGGCTCTGAAACCCCTGCCTCGA |
| | EITRTTLQSDQEEIDY | GCAACACTGGTGATTATTACGAGGACAGTTATGAAGATATTTC |
| | DDTISVEMKKEDFDI | AGCATACTTGCTGAGTAAAAACAATGCCATTGAACCAAGAAG |
| | YDEDENQSPRSFQKK | CTTCTCTCAAAACCCACCAGTCTTGAAACGCCATCAACGGGAA |
| | TRHYFIAAVERLWDY | ATAACTCGTACTACTCTTCAGTCAGATCAAGAGGAAATCGATT |
| | GMSSSPHVLRNRAQS | ATGATGATACCATATCAGTTGAAATGAAGAAGGAAGATTTTG |
| | GSVPQFKKVVFQEFT | ACATTTATGATGAGGATGAAAATCAGAGCCCCCGCAGCTTTCA |
| | DGSFTQPLYRGELNE | AAAGAAAACACGACACTATTTTATTGCTGCAGTGGAGAGGCTC |
| | HLGLLGPYIRAEVED | TGGGATTATGGGATGAGTAGCTCCCCACATGTTCTAAGAAACA |
| | NIMVTFRNQASRPYS | GGGCTCAGAGTGGCAGTGTCCCTCAGTTCAAGAAAGTTGTTTT |
| | FYSSLISYEEDQRQGA | CCAGGAATTTACTGATGGCTCCTTTACTCAGCCCTTATACCGTG |
| | EPRKNFVKPNETKTY | GAGAACTAAATGAACATTTGGGACTCCTGGGGCCATATATAA |
| | FWKVQHHMAPTKDE | GAGCAGAAGTTGAAGATAATATCATGGTAACTTTCAGAAATC |
| | FDCKAWAYFSDVDL | AGGCCTCTCGTCCCTATTCCTTCTATTCTAGCCTTATTTCTTATG |
| | EKDVHSGLIGPLLVC | AGGAAGATCAGAGGCAAGGAGCAGAACCTAGAAAAAACTTTG |
| | HTNTLNPAHGRQVTV | TCAAGCCTAATGAAACCAAAACTTACTTTTGGAAAGTGCAACA |
| | QEFALFFTIFDETKSW | TCATATGGCACCCACTAAAGATGAGTTTGACTGCAAAGCCTGG |
| | YFTENMERNCRAPCN | GCTTATTTCTCTGATGTTGACCTGGAAAAAGATGTGCACTCAG |
| | IQMEDPTFKENYRFH | GCCTGATTGGACCCCTTCTGGTCTGCCACACTAACACACTGAA |
| | AINGYIMDTLPGLVM | CCCTGCTCATGGGAGACAAGTGACAGTACAGGAATTTGCTCTG |
| | AQDQRIRWYLLSMGS | TTTTTCACCATCTTTGATGAGACCAAAAGCTGGTACTTCACTG |
| | NENIHSIHFSGHVFTV | AAAATATGGAAAGAAACTGCAGGGCTCCCTGCAATATCCAGA |
| | RKKEEYKMALYNLY | TGGAAGATCCCACTTTTAAAGAGAATTATCGCTTCCATGCAAT |
| | PGVFETVEMLPSKAG | CAATGGCTACATAATGGATACACTACCTGGCTTAGTAATGGCT |
| | IWRVECLIGEHLHAG | CAGGATCAAAGGATTCGATGGTATCTGCTCAGCATGGGCAGC |
| | MSTLFLVYSNKCQTP | AATGAAAACATCCATTCTATTCATTTCAGTGGACATGTGTTCA |
| | LGMASGHIRDFQITAS | CTGTACGAAAAAAAGAGGAGTATAAAATGGCACTGTACAATC |
| | GQYGQWAPKLARLH | TCTATCCAGGTGTTTTTGAGACAGTGGAAATGTTACCATCCAA |
| | YSGSINAWSTKEPFS | AGCTGGAATTTGGCGGGTGGAATGCCTTATTGGCGAGCATCTA |
| | WIKVDLLAPMIIHGIK | CATGCTGGGATGAGCACACTTTTTCTGGTGTACAGCAATAAGT |
| | TQGARQKFSSLYISQF | GTCAGACTCCCCTGGGAATGGCTTCTGGACACATTAGAGATTT |
| | IIMYSLDGKKWQTYR | TCAGATTACAGCTTCAGGACAATATGGACAGTGGGCCCCAAA |
| | GNSTGTLMVFFGNVD | GCTGGCCAGACTTCATTATTCCGGATCAATCAATGCCTGGAGC |
| | SSGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0135 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNGAPG | GGAACAGAGGCATGACCGCCTTACTGAAGGTTTCTAGTTGTGA |
| | SPAGSPTSTEEGTSES | CAAGAACACTGGTGATTATTACGAGGACAGTTATGAAGATATT |
| | ATPESGPGSEPATSGS | TCAGCATACTTGCTGAGTAAAAACAATGCCATTGAACCAAGA |
| | ETPASSPPVLKRHQRE | AGCTTCTCTCAAAACGGCGCGCCAGGTTCTCCTGCTGGCTCCC |
| | ITRTTLQSDQEEIDYD | CCACCTCAACAGAAGAGGGGACAAGCGAAAGCGCTACGCCTG |
| | DTISVEMKKEDFDIY | AGAGTGGCCCTGGCTCTGAGCCAGCCACCTCCGGCTCTGAAAC |
| | DEDENQSPRSFQKKT | CCCTGCCTCGAGCCCACCAGTCTTGAAACGCCATCAACGGGAA |
| | RHYFIAAVERLWDYG | ATAACTCGTACTACTCTTCAGTCAGATCAAGAGGAAATCGATT |
| | MSSSPHVLRNRAQSG | ATGATGATACCATATCAGTTGAAATGAAGAAGGAAGATTTTG |
| | SVPQFKKVVFQEFTD | ACATTTATGATGAGGATGAAAATCAGAGCCCCCGCAGCTTTCA |
| | GSFTQPLYRGELNEH | AAAGAAAACACGACACTATTTTATTGCTGCAGTGGAGAGGCTC |
| | LGLLGPYIRAEVEDNI | TGGGATTATGGGATGAGTAGCTCCCCACATGTTCTAAGAAACA |
| | MVTFRNQASRPYSFY | GGGCTCAGAGTGGCAGTGTCCCTCAGTTCAAGAAAGTTGTTTT |
| | SSLISYEEDQRQGAEP | CCAGGAATTTACTGATGGCTCCTTTACTCAGCCCTTATACCGTG |
| | RKNFVKPNETKTYFW | GAGAACTAAATGAACATTTGGGACTCCTGGGGCCATATATAA |
| | KVQHHMAPTKDEFD | GAGCAGAAGTTGAAGATAATATCATGGTAACTTTCAGAAATC |
| | CKAWAYFSDVDLEK | AGGCCTCTCGTCCCTATTCCTTCTATTCTAGCCTTATTTCTTATG |
| | DVHSGLIGPLLVCHT | AGGAAGATCAGAGGCAAGGAGCAGAACCTAGAAAAAACTTTG |
| | NTLNPAHGRQVTVQE | TCAAGCCTAATGAAACCAAAACTTACTTTTGGAAAGTGCAACA |
| | FALFFTIFDETKSWYF | TCATATGGCACCCACTAAAGATGAGTTTGACTGCAAAGCCTGG |
| | TENMERNCRAPCNIQ | GCTTATTTCTCTGATGTTGACCTGGAAAAAGATGTGCACTCAG |
| | MEDPTFKENYRFHAI | GCCTGATTGGACCCCTTCTGGTCTGCCACACTAACACACTGAA |
| | NGYIMDTLPGLVMA | CCCTGCTCATGGGAGACAAGTGACAGTACAGGAATTTGCTCTG |
| | QDQRIRWYLLSMGSN | TTTTTCACCATCTTTGATGAGACCAAAAGCTGGTACTTCACTG |
| | ENIHSIHFSGHVFTVR | AAAATATGGAAAGAAACTGCAGGGCTCCCTGCAATATCCAGA |
| | KKEEYKMALYNLYP | TGGAAGATCCCACTTTTAAAGAGAATTATCGCTTCCATGCAAT |
| | GVFETVEMLPSKAGI | CAATGGCTACATAATGGATACACTACCTGGCTTAGTAATGGCT |
| | WRVECLIGEHLHAG | CAGGATCAAAGGATTCGATGGTATCTGCTCAGCATGGGCAGC |
| | MSTLFLVYSNKCQTP | AATGAAAACATCCATTCTATTCATTTCAGTGGACATGTGTTCA |
| | LGMASGHIRDFQITAS | CTGTACGAAAAAAAGAGGAGTATAAAATGGCACTGTACAATC |
| | GQYGQWAPKLARLH | TCTATCCAGGTGTTTTTGAGACAGTGGAAATGTTACCATCCAA |
| | YSGSINAWSTKEPFS | AGCTGGAATTTGGCGGGTGGAATGCCTTATTGGCGAGCATCTA |
| | WIKVDLLAPMIIHGIK | CATGCTGGGATGAGCACACTTTTTCTGGTGTACAGCAATAAGT |
| | TQGARQKFSSLYISQF | GTCAGACTCCCCTGGGAATGGCTTCTGGACACATTAGAGATTT |
| | IIMYSLDGKKWQTYR | TCAGATTACAGCTTCAGGACAATATGGACAGTGGGCCCCAAA |
| | GNSTGTLMVFFGNVD | GCTGGCCAGACTTCATTATTCCGGATCAATCAATGCCTGGAGC |
| | SSGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0135-2 | MQIELSTCFFLCLL | atgcaaatagagctctccacctgcttctttctgtgccttttgcgattctgctttagtgccaccagaagatacta |
| | RFCFSATRRYYLGA | cctgggtgcagtggaactgtcatgggactatatgcaaagtgatctcggtgagctgcctgtggacgcaag |
| | VELSWDYMQSDLG | atttcctcctagagtgccaaaatcttttccattcaacacctcagtcgtgtacaaaaagactctgtttgtagaat |
| | ELPVDARFPPRVPK | tcacggatcaccttttcaacatcgctaagccaaggccaccctggatgggtctgctaggtcctaccatcca |
| | SFPFNTSVVYKKTL | ggctgaggtttatgatacagtggtcattacacttaagaacatggcttcccatcctgtcagtcttcatgctgtt |
| | FVEFTDHLFNIAKP | ggtgtatcctactggaaagcttctgagggagctgaatatgatgatcagaccagtcaaagggagaaagaa |
| | RPPWMGLLGPTIQA | gatgataaagtcttccctggtggaagccatacatatgtctggcaggtcctgaaagagaatggtccaatgg |
| | EVYDTVVITLKNM | cctctgacccactgtgccttacctactcatatctttctcatgtggacctggtaaaagacttgaattcaggcct |
| | ASHPVSLHAVGVS | cattggagccctactagtatgtagagaagggagtctggccaaggaaaagacacagaccttgcacaaatt |
| | YWKASEGAEYDDQ | tatactactttttgctgtatttgatgaagggaaaagttggcactcagaaacaaagaactccttgatgcagga |
| | TSQREKEDDKVFPG | tagggatgctgcatctgctcgggcctggcctaaaatgcacacagtcaatggttatgtaaacaggtctctg |
| | GSHTYVWQVLKEN | ccaggtctgattggatgccacaggaaatcagtctattggcatgtgattggaatgggcaccactcctgaag |
| | GPMASDPLCLTYSY | tgcactcaatattcctcgaaggtcacacatttcttgtgaGGAACCATCGCCAGGCTAGC |
| | LSHVDLVKDLNSG | TTGGAAATCTCGCCAATAACTttccttactgctcaaacactcttgatggaccttggaca |
| | LIGALLVCREGSLA | gtttctactgttttgtcatatctcttcccaccaacatgatggcatggaagcttatgtcaaagtagacagctgtc |
| | KEKTQTLHKFILLF | cagaggaaccccaactacgaatgaaaaataatgaagaagcggaagactatgatgatgatcttactgatt |
| | AVFDEGKSWHSET | ctgaaatggatgtggtcaggtttgatgatgacaactctccttcctttatccaaattcgctcagttgccaagaa |
| | KNSLMQDRDAASA | gcatcctaaaacttgggtacattacattgctgctgaagaggaggactgggactatgctcccttagtcctcg |
| | RAWPKMHTVNGY | cccccgatgacagaagttataaaagtcaatatttgaacaatggccctcagcggattggtaggaagtacaa |
| | VNRSLPGLIGCHRK | aaaagtccgatttatggcatacacagatgaaacctttaagactcgtgaagctattcagcatgaatcaggaa |
| | SVYWHVIGMGTTP | tcttgggacctttactttatggggaagttggagacacactgttgattatatttaagaatcaagcaagcagac |
| | EVHSIFLEGHTFLV | catataacatctaccctcacggaatcactgatgtccgtcctttgtattcaaggagattaccaaaaggtgtaa |
| | RNHRQASLEISPITF | aacatttgaaggattttccaattctgccaggagaaatattcaaatataaatggacagtgactgtagaagatg |
| | LTAQTLLMDLGQF | ggccaactaaatcagatcctcggtgcctgacccgctattactctagtttcgttaatatggagagagatcta |
| | LLFCHISSHQHDGM | gcttcaggactcattggccctctcctcatctgctacaaagaatctgtagatcaaagaggaaaccagataat |
| | EAYVKVDSCPEEPQ | gtcagacaagaggaatgtcatcctgttttctgtatttgatgagaaccgaagctggtacctcacagagaata |
| | LRMKNNEEAEDYD | tacaacgctttctccccaatccagctggagtgcagcttgaggatccagagttccaagcctccaacatcat |
| | DDLTDSEMDVVRF | gcacagcatcaatggctatgtttttgatagtttgcagttgtcagtttgtttgcatgaggtggcatactggtaca |
| | DDDNSPSFIQIRSVA | ttctaagcattggagcacagactgacttcctttctgtcttcttctctggatataccttcaaacacaaaatggtc |
| | KKHPKTWVHYIAA | tatgaagacacactcaccctattcccattctcaggagaaactgtcttcatgtcgatggaaaacccaggtct |
| | EEEDWDYAPLVLA | atggattctggggtgccacaactcagactttcggaacagaggcatgaccgccttactgaaggtttctagtt |
| | PDDRSYKSQYLNN | gtgacaagaacactggtgattattacgaggacagttatgaagatatttcagcatacttgctgagtaaaaac |
| | GPQRIGRKYKKVRF | aatgccattgaaccaagaagcttctctGGCGCGCCAGGTTCTCCTGCTGGCTCC |
| | MAYTDETFKTREAI | CCCACCTCAACAGAAGAGGGGACAAGcgaaagcgctacgcctgagaGTG |
| | QHESGILGPLLYGE | GCCCTGGCTCTGAGCCAGCCACCTCCGGCTCTGAAACCCCTGC |
| | VGDTLLIIFKNQAS | CTCGAGCcaaaacccaccagtcttgaaacgccatcaacgggaaataactcgtactacTCTTC |
| | RPYNIYPHGITDVR | AGTCAGATCAAGAGGAAATCGATTATGATGATACCATATCAGT |
| | PLYSRRLPKGVKHL | TGAAATGAAgaaggaagattttgacatttatgatgaggatgaaaatcagagcccccgcagcttt |
| | KDFPILPGEIFKYK | caaaagaaaacacgacactattttattgctgcagtggagaggctctgggattatgggatgagtagctccc |
| | WTVTVEDGPTKSD | cacatgttctaagaaacagggctcagagtggcagtgtccctcagttcaagaaagttgttttccaggaattt |
| | PRCLTRYYSSFVNM | actgatggctcctttactcagcccttataccgtggagaactaaatgaacatttgggactcctggggccata |
| | ERDLASGLIGPLLIC | tataagagcagaagttgaagataatatcatggtaactttcagaaatcaggcctctcgtccctattccttctat |
| | YKESVDQRGNQIM | tctagccttatttcttatgaggaagatcagaggcaaggagcagaacctagaaaaaactttgtcaagccta |
| | SDKRNVILFSVFDE | atgaaaccaaaacttacttttggaaagtgcaacatcatatggcacccactaaagatgagtttgactgcaaa |
| | NRSWYLTENIQRFL | gcctgggcttatttctctgatgttgacctggaaaaagatgtgcactcaggcctgattggaccccttctggtc |
| | PNPAGVQLEDPEFQ | tgccacactaacacactgaaccctgctcatgggagacaagtgacagtacaggaatttgctctgtttttcac |
| | ASNIMHSINGYVFD | catctttgatgagaccaaaagctggtacttcactgaaaatatggaaagaaactgcagggctccctgcaat |
| | SLQLSVCLHEVAY | atccagatggaagatcccacttttaaagagaattatcgcttccatgcaatcaatggctacataatggataca |
| | WYILSIGAQTDFLS | ctacctggcttagtaatggctcaggatcaaaggattcgatggtatctgctcagcatgggcagcaatgaaa |
| | VFFSGYTFKHKMV | acatccattctattcatttcagtggacatgtgttcactgtacgaaaaaaagaggagtataaaatggcactgt |
| | YEDTLTLFPFSGET | acaatctctatccaggtgtttttgagacagtggaaatgttaccatccaaagctggaatttggcgggtggaa |
| | VFMSMENPGLWIL | tgccttattggcgagcatctacatgctgggatgagcacactttttctggtgtACAGCAATAAGT |
| | GCHNSDFRNRGMT | GTCAGACTCCcctgggaatggcttctggacacattagagattttcagattacagcttcaggacaa |
| | ALLKVSSCDKNTG | tatggacagtgggccccaaagctggccagacttcattattccggatcaatcaatgcctggagcaccaag |
| | DYYEDSYEDISAYL | gagcccttttcttggatcaaggtggatctgttggcaccaatgattattcacggcatcaagacccagggtgc |
| | LSKNNAIEPRSFSG | ccgtcagaagttctccagcctctacatctctcagtttatcatcatgtatagtcttgatgggaagaagtggca |
| | APGSPAGSPTSTEE | gacttatcgaggaaattccactggaaccttaatggtcttctttggcaatgtggattcatctgggataaaaca |
| | GTSESATPESGPGS | caatatttttaaccctccaattattgctcgatacatccgtttgcacccaactcattatagcattcgcagcactc |
| | EPATSGSETPASSQ | ttcgcatggagttgatgggctgtgatttaaatagttgcagcatgccattgggaatggagagtaaagcaata |
| | NPPVLKRHQREITR | tcagatgcacagattactgcttcatcctactttaccaatatgtttgccacctggtctccttcaaaagctcgact |
| | TTLQSDQEEIDYDD | tcacctccaagggaggagtaatgcctggagacctcaggtgaataatccaaaagagtggctgcaagtgg |
| | TISVEMKKEDFDIY | acttccagaagacaatgaaagtcacaggagtaactactcagggagtaaaatctctgcttaccagcatgta |
| | DEDENQSPRSFQKK | tgtgaaggagttcctcatctccagcagtcaagatggccatcagtggactctcttttttcagaatggcaaagt |
| | TRHYFIAAVERLW | aaaggtttttcagggaaatcaagactccttcacacctgtggtgaactctctagacccaccgttactgactc |
| | DYGMSSSPHVLRN | gctaccttcgaattcacccccagagttgggtgcaccagattgccctgaggatggaggttctgggctgcg |
| | RAQSGSVPQFKKV | aggcacaggacctctacggcgccggatcacctggggccgaaacggccgaacaaaaactcatctcag |
| | VFQEFTDGSFTQPL | aagaggatctgtcacctgcaaccggttga |
| | YRGELNEHLGLLGP | |
| | YIRAEVEDNIMVTF | |
| | RNQASRPYSFYSSLI | |
| | SYEEDQRQGAEPR | |
| | KNFVKPNETKTYF | |
| | WKVQHHMAPTKD | |
| | EFDCKAWAYFSDV | |
| | DLEKDVHSGLIGPL | |
| | LVCHTNTLNPAHG | |
| | RQVTVQEFALFFTI | |
| | FDETKSWYFTENM | |
| | ERNCRAPCNIQME | |
| | DPTFKENYRFHAIN | |
| | GYIMDTLPGLVMA | |
| | QDQRIRWYLLSMG | |
| | SNENIHSIHFSGHVF | |
| | TVRKKEEYKMALY | |
| | NLYPGVFETVEMLP | |
| | SKAGIWRVECLIGE | |
| | HLHAGMSTLFLVY | |
| | SNKCQTPLGMASG | |
| | HIRDFQITASGQYG | |
| | QWAPKLARLHYSG | |
| | SINAWSTKEPFSWI | |
| | KVDLLAPMIIHGIK | |
| | TQGARQKFSSLYIS | |
| | QFIIMYSLDGKKW | |
| | QTYRGNSTGTLMV | |
| | FFGNVDSSGIKHNIF | |
| | NPPIIARYIRLHPTH | |
| | YSIRSTLRMELMGC | |
| | DLNSCSMPLGMES | |
| | KAISDAQITASSYFT | |
| | NMFATWSPSKARL | |
| | HLQGRSNAWRPQV | |
| | NNPKEWLQVDFQK | |
| | TMKVTGVTTQGVK | |
| | SLLTSMYVKEFLISS | |
| | SQDGHQWTLFFQN | |
| | GKVKVFQGNQDSF | |
| | TPVVNSLDPPLLTR | |
| | YLRIHPQSWVHQIA | |
| | LRMEVLGCEAQDL | |
| | YGAGSPGAETAEQ | |
| | KLISEEDLSPATG | |
| pBC0149 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNGAPG | GGAACAGAGGCATGACCGCCTTACTGAAGGTTTCTAGTTGTGA |
| | SPAGSPTSTEEGTSES | CAAGAACACTGGTGATTATTACGAGGACAGTTATGAAGATATT |
| | ATPESGPGSEPATSGS | TCAGCATACTTGCTGAGTAAAAACAATGCCATTGAACCAAGA |
| | ETPASSPPVLKRHQA | AGCTTCTCTCAAAACGGCGCGCCAGGTTCTCCTGCTGGCTCCC |
| | EITRTTLQSDQEEIDY | CCACCTCAACAGAAGAGGGGACAAGCGAAAGCGCTACGCCTG |
| | DDTISVEMKKEDFDI | AGAGTGGCCCTGGCTCTGAGCCAGCCACCTCCGGCTCTGAAAC |
| | YDEDENQSPRSFQKK | CCCTGCCTCGAGCCCACCAGTCTTGAAACGCCATCAAGCTGAA |
| | TRHYFIAAVERLWDY | ATAACTCGTACTACTCTTCAGTCAGATCAAGAGGAAATCGATT |
| | GMSSSPHVLRNRAQS | ATGATGATACCATATCAGTTGAAATGAAGAAGGAAGATTTTG |
| | GSVPQFKKVVFQEFT | ACATTTATGATGAGGATGAAAATCAGAGCCCCCGCAGCTTTCA |
| | DGSFTQPLYRGELNE | AAAGAAAACACGACACTATTTTATTGCTGCAGTGGAGAGGCTC |
| | HLGLLGPYIRAEVED | TGGGATTATGGGATGAGTAGCTCCCCACATGTTCTAAGAAACA |
| | NIMVTFRNQASRPYS | GGGCTCAGAGTGGCAGTGTCCCTCAGTTCAAGAAAGTTGTTTT |
| | FYSSLISYEEDQRQGA | CCAGGAATTTACTGATGGCTCCTTTACTCAGCCCTTATACCGTG |
| | EPRKNFVKPNETKTY | GAGAACTAAATGAACATTTGGGACTCCTGGGGCCATATATAA |
| | FWKVQHHMAPTKDE | GAGCAGAAGTTGAAGATAATATCATGGTAACTTTCAGAAATC |
| | FDCKAWAYFSDVDL | AGGCCTCTCGTCCCTATTCCTTCTATTCTAGCCTTATTTCTTATG |
| | EKDVHSGLIGPLLVC | AGGAAGATCAGAGGCAAGGAGCAGAACCTAGAAAAAACTTTG |
| | HTNTLNPAHGRQVTV | TCAAGCCTAATGAAACCAAAACTTACTTTTGGAAAGTGCAACA |
| | QEFALFFTIFDETKSW | TCATATGGCACCCACTAAAGATGAGTTTGACTGCAAAGCCTGG |
| | YFTENMERNCRAPCN | GCTTATTTCTCTGATGTTGACCTGGAAAAAGATGTGCACTCAG |
| | IQMEDPTFKENYRFH | GCCTGATTGGACCCCTTCTGGTCTGCCACACTAACACACTGAA |
| | AINGYIMDTLPGLVM | CCCTGCTCATGGGAGACAAGTGACAGTACAGGAATTTGCTCTG |
| | AQDQRIRWYLLSMGS | TTTTTCACCATCTTTGATGAGACCAAAAGCTGGTACTTCACTG |
| | NENIHSIHFSGHVFTV | AAAATATGGAAAGAAACTGCAGGGCTCCCTGCAATATCCAGA |
| | RKKEEYKMALYNLY | TGGAAGATCCCACTTTTAAAGAGAATTATCGCTTCCATGCAAT |
| | PGVFETVEMLPSKAG | CAATGGCTACATAATGGATACACTACCTGGCTTAGTAATGGCT |
| | IWRVECLIGEHLHAG | CAGGATCAAAGGATTCGATGGTATCTGCTCAGCATGGGCAGC |
| | MSTLFLVYSNKCQTP | AATGAAAACATCCATTCTATTCATTTCAGTGGACATGTGTTCA |
| | LGMASGHIRDFQITAS | CTGTACGAAAAAAAGAGGAGTATAAAATGGCACTGTACAATC |
| | GQYGQWAPKLARLH | TCTATCCAGGTGTTTTTGAGACAGTGGAAATGTTACCATCCAA |
| | YSGSINAWSTKEPFS | AGCTGGAATTTGGCGGGTGGAATGCCTTATTGGCGAGCATCTA |
| | WIKVDLLAPMIIHGIK | CATGCTGGGATGAGCACACTTTTTCTGGTGTACAGCAATAAGT |
| | TQGARQKFSSLYISQF | GTCAGACTCCCCTGGGAATGGCTTCTGGACACATTAGAGATTT |
| | IIMYSLDGKKWQTYR | TCAGATTACAGCTTCAGGACAATATGGACAGTGGGCCCCAAA |
| | GNSTGTLMVFFGNVD | GCTGGCCAGACTTCATTATTCCGGATCAATCAATGCCTGGAGC |
| | SSGIKHNIFNPPIIARYI | ACCAAGGAGCCCTTTTCTTGGATCAAGGTGGATCTGTTGGCAC |
| | RLHPTHYSIRSTLRME | CAATGATTATTCACGGCATCAAGACCCAGGGTGCCCGTCAGAA |
| | LMGCDLNSCSMPLG | GTTCTCCAGCCTCTACATCTCTCAGTTTATCATCATGTATAGTC |
| | MESKAISDAQITASSY | TTGATGGGAAGAAGTGGCAGACTTATCGAGGAAATTCCACTG |
| | FTNMFATWSPSKARL | GAACCTTAATGGTCTTCTTTGGCAATGTGGATTCATCTGGGAT |
| | HLQGRSNAWRPQVN | AAAACACAATATTTTTAACCCTCCAATTATTGCTCGATACATC |
| | NPKEWLQVDFQKTM | CGTTTGCACCCAACTCATTATAGCATTCGCAGCACTCTTCGCAT |
| | KVTGVTTQGVKSLLT | GGAGTTGATGGGCTGTGATTTAAATAGTTGCAGCATGCCATTG |
| | SMYVKEFLISSSQDG | GGAATGGAGAGTAAAGCAATATCAGATGCACAGATTACTGCT |
| | HQWTLFFQNGKVKV | TCATCCTACTTTACCAATATGTTTGCCACCTGGTCTCCTTCAAA |
| | FQGNQDSFTPVVNSL | AGCTCGACTTCACCTCCAAGGGAGGAGTAATGCCTGGAGACCT |
| | DPPLLTRYLRIHPQSW | CAGGTGAATAATCCAAAAGAGTGGCTGCAAGTGGACTTCCAG |
| | VHQIALRMEVLGCEA | AAGACAATGAAAGTCACAGGAGTAACTACTCAGGGAGTAAAA |
| | QDLYGAGSPGAETAE | TCTCTGCTTACCAGCATGTATGTGAAGGAGTTCCTCATCTCCA |
| | QKLISEEDLSPATG | GCAGTCAAGATGGCCATCAGTGGACTCTCTTTTTTCAGAATGG |
| | | CAAAGTAAAGGTTTTTCAGGGAAATCAAGACTCCTTCACACCT |
| | | GTGGTGAACTCTCTAGACCCACCGTTACTGACTCGCTACCTTC |
| | | GAATTCACCCCCAGAGTTGGGTGCACCAGATTGCCCTGAGGAT |
| | | GGAGGTTCTGGGCTGCGAGGCACAGGACCTCTACGGCGCCGG |
| | | ATCACCTGGGGCCGAAACGGCCGAACAAAAACTCATCTCAGA |
| | | AGAGGATCTGTCACCTGCAACCGGTTGA |
| pBC0149-2 | MQIELSTCFFLCLL | atgcaaatagagctctccacctgcttctttctgtgccttttgcgattctgctttagtgccaccagaa |
| | RFCFSATRRYYLGA | gatactacctgggtgcagtggaactgtcatgggactatatgcaaagtgatctcggtgagctgc |
| | VELSWDYMQSDLG | ctgtggacgcaagatttcctcctagagtgccaaaatcttttccattcaacacctcagtcgtgtaca |
| | ELPVDARFPPRVPK | aaaagactctgtttgtagaattcacggatcaccttttcaacatcgctaagccaaggccaccctg |
| | SFPFNTSVVYKKTL | gatgggtctgctaggtcctaccatccaggctgaggtttatgatacagtggtcattacacttaaga |
| | FVEFTDHLFNIAKP | acatggcttcccatcctgtcagtcttcatgctgttggtgtatcctactggaaagcttctgagggag |
| | RPPWMGLLGPTIQA | ctgaatatgatgatcagaccagtcaaagggagaaagaagatgataaagtcttccctggtggaa |
| | EVYDTVVITLKNM | gccatacatatgtctggcaggtcctgaaagagaatggtccaatggcctctgacccactgtgcct |
| | ASHPVSLHAVGVS | tacctactcatatctttctcatgtggacctggtaaaagacttgaattcaggcctcattggagccct |
| | YWKASEGAEYDDQ | actagtatgtagagaagggagtctggccaaggaaaagacacagaccttgcacaaatttatact |
| | TSQREKEDDKVFPG | actttttgctgtatttgatgaagggaaaagttggcactcagaaacaaagaactccttgatgcagg |
| | GSHTYVWQVLKEN | atagggatgctgcatctgctcgggcctggcctaaaatgcacacagtcaatggttatgtaaaca |
| | GPMASDPLCLTYSY | ggtctctgccaggtctgattggatgccacaggaaatcagtctattggcatgtgattggaatggg |
| | LSHVDLVKDLNSG | caccactcctgaagtgcactcaatattcctcgaaggtcacacatttcttgtgaGGAACCA |
| | LIGALLVCREGSLA | TCGCCAGGCTAGCTTGGAAATCTCGCCAATAACTttccttactg |
| | KEKTQTLHKFILLF | ctcaaacactcttgatggaccttggacagtttctactgttttgtcatatctcttcccaccaacatgat |
| | AVFDEGKSWHSET | ggcatggaagcttatgtcaaagtagacagctgtccagaggaaccccaactacgaatgaaaaa |
| | KNSLMQDRDAASA | taatgaagaagcggaagactatgatgatgatcttactgattctgaaatggatgtggtcaggtttg |
| | RAWPKMHTVNGY | atgatgacaactctccttcctttatccaaattcgctcagttgccaagaagcatcctaaaacttggg |
| | VNRSLPGLIGCHRK | tacattacattgctgctgaagaggaggactgggactatgctcccttagtcctcgcccccgatga |
| | SVYWHVIGMGTTP | cagaagttataaaagtcaatatttgaacaatggccctcagcggattggtaggaagtacaaaaa |
| | EVHSIFLEGHTFLV | agtccgatttatggcatacacagatgaaacctttaagactcgtgaagctattcagcatgaatcag |
| | RNHRQASLEISPITF | gaatcttgggacctttactttatggggaagttggagacacactgttgattatatttaagaatcaag |
| | LTAQTLLMDLGQF | caagcagaccatataacatctaccctcacggaatcactgatgtccgtcctttgtattcaaggaga |
| | LLFCHISSHQHDGM | ttaccaaaaggtgtaaaacatttgaaggattttccaattctgccaggagaaatattcaaatataaa |
| | EAYVKVDSCPEEPQ | tggacagtgactgtagaagatgggccaactaaatcagatcctcggtgcctgacccgctattact |
| | LRMKNNEEAEDYD | ctagtttcgttaatatggagagagatctagcttcaggactcattggccctctcctcatctgctaca |
| | DDLTDSEMDVVRF | aagaatctgtagatcaaagaggaaaccagataatgtcagacaagaggaatgtcatcctgttttc |
| | DDDNSPSFIQIRSVA | tgtatttgatgagaaccgaagctggtacctcacagagaatatacaacgctttctccccaatcca |
| | KKHPKTWVHYIAA | gctggagtgcAGCTTGAGGATCCAGAGTTCcaagcctccaacatcatgca |
| | EEEDWDYAPLVLA | cagcatcaatggctatgtttttgatagtttgcagttgtcagtttgtttgcatgaggtggcatactggt |
| | PDDRSYKSQYLNN | acattctaagcattggagcacagactgacttcctttctgtcttcttctctggatataccttcaaaca |
| | GPQRIGRKYKKVRF | caaaatggtctatgaagacacactcaccctattcccattctcaggagaaactgtcttcatgtcga |
| | MAYTDETFKTREAI | tggaaaacccaggtctatggattctggggtgccacaactcagactttcggaacagaggcatga |
| | QHESGILGPLLYGE | ccgccttactgaaggtttctagttgtgacaagaacactggtgattattacgaggacagttatgaa |
| | VGDTLLIIFKNQAS | gatatttcagcatacttgctgagtaaaaacaatgccattgaaccaagaagcttctctGGCGC |
| | RPYNIYPHGITDVR | GCCAGGTTCTCCTGCTGGCTCCCCCACCTCAACAGAAGA |
| | PLYSRRLPKGVKHL | GGGGACAAGcgaaagcgctacgcctgagaGTGGCCCTGGCTCTGA |
| | KDFPILPGEIFKYK | GCCAGCCACCTCCGGCTCTGAAACCCCTGCCTCGAGCcaa |
| | WTVTVEDGPTKSD | aacccaccagtcttgaaacgccatcaaGCTgaaataactcgtactacTCTTCAGTC |
| | PRCLTRYYSSFVNM | AGATCAAGAGGAAATCGATTATGATGATACCATATCAGT |
| | ERDLASGLIGPLLIC | TGAAATGAAgaaggaagattttgacatttatgatgaggatgaaaatcagagcccccg |
| | YKESVDQRGNQIM | cagctttcaaaagaaaacacgacactattttattgctgcagtggagaggctctgggattatggg |
| | SDKRNVILFSVFDE | atgagtagctccccacatgttctaagaaacagggctcagagtggcagtgtccctcagttcaag |
| | NRSWYLTENIQRFL | aaagttgttttccaggaatttactgatggctcctttactcagcccttataccgtggagaactaaatg |
| | PNPAGVQLEDPEFQ | aacatttgggactcctggggccatatataagagcagaagttgaagataatatcatggtaactttc |
| | ASNIMHSINGYVFD | agaaatcaggcctctcgtccctattccttctattctagccttatttcttatgaggaagatcagaggc |
| | SLQLSVCLHEVAY | aaggagcagaacctagaaaaaactttgtcaagcctaatgaaaccaaaacttacttttggaaagt |
| | WYILSIGAQTDFLS | gcaacatcatatggcacccactaaagatgagtttgactgcaaagcctgggcttatttctctgatg |
| | VFFSGYTFKHKMV | ttgacctggaaaaagatgtgcactcaggcctgattggaccccttctggtctgccacactaacac |
| | YEDTLTLFPFSGET | actgaaccctgctcatgggagacaagtgacagtacaggaatttgctctgtttttcaccatctttga |
| | VFMSMENPGLWIL | TGAGACCAAAAGCTGGTACTTCactgaaaatatggaaagaaactgcagg |
| | GCHNSDFRNRGMT | gctccctgcaatatccagatggaagatcccacttttaaagagaattatcgcttccatgcaatcaa |
| | ALLKVSSCDKNTG | tggctacataatggatacactacctggcttagtaatggctcaggatcaaaggattcgatggtatc |
| | DYYEDSYEDISAYL | tgctcagcatgggcagcaatgaaaacatccattctattcatttcagtggacatgtgttcactgtac |
| | LSKNNAIEPRSFSG | gaaaaaaagaggagtataaaatggcactgtacaatctctatccaggtgtttttgagacagtgga |
| | APGSPAGSPTSTEE | aatgttaccatccaaagctggaatttggcgggtggaatgccttattggcgagcatctacatgct |
| | GTSESATPESGPGS | gggatgagcacactttttctggtgtACAGCAATAAGTGTCAGACTCCcctg |
| | EPATSGSETPASSQ | ggaatggcttctggacacattagagattttcagattacagcttcaggacaatatggacagtggg |
| | NPPVLKRHQAEITR | ccccaaagctggccagacttcattattccggatcaatcaatgcctggagcaccaaggagccct |
| | TTLQSDQEEIDYDD | tttcttggatcaaggtggatctgttggcaccaatgattattcacggcatcaagacccagggtgcc |
| | TISVEMKKEDFDIY | cgtcagaagttctccagcctctacatctctcagtttatcatcatgtatagtcttgatgggaagaagt |
| | DEDENQSPRSFQKK | ggcagacttatcgaggaaattccactggaaccttaatggtcttctttggcaatgtggattcatctg |
| | TRHYFIAAVERLW | ggataaaacacaatatttttaaccctccaattattgctcgatacatccgtttgcacccaactcatta |
| | DYGMSSSPHVLRN | tagcattcgcagcactcttcgcatggagttgatgggctgtgatttaaatagttgcagcatgccatt |
| | RAQSGSVPQFKKV | gggaatggagagtaaagcaatatcagatgcacagattactgcttcatcctactttaccaatatgt |
| | VFQEFTDGSFTQPL | ttgccacctggtctccttcaaaagctcgacttcacctccaagggaggagtaatgcctggagac |
| | YRGELNEHLGLLGP | ctcaggtgaataatccaaaagagtggctgcaagtggacttccagaagacaatgaaagtcaca |
| | YIRAEVEDNIMVTF | ggagtaactactcagggagtaaaatctctgcttaccagcatgtatgtgaaggagttcctcatctc |
| | RNQASRPYSFYSSLI | cagcagtcaagatggccatcagtggactctcttttttcagaatggcaaagtaaaggtttttcagg |
| | SYEEDQRQGAEPR | gaaatcaagactccttcacacctgtggtgaactctctagacccaccgttactgactcgctacctt |
| | KNFVKPNETKTYF | cgaattcacccccagagttgggtgcaccagattgccctgaggatggaggttctgggctgcga |
| | WKVQHHMAPTKD | ggcacaggacctctacggcgccggatcacctggggccgaaacggccgaacaaaaactcat |
| | EFDCKAWAYFSDV | ctcagaagaggatctgtcacctgcaaccggttga |
| | DLEKDVHSGLIGPL | |
| | LVCHTNTLNPAHG | |
| | RQVTVQEFALFFTI | |
| | FDETKSWYFTENM | |
| | ERNCRAPCNIQME | |
| | DPTFKENYRFHAIN | |
| | GYIMDTLPGLVMA | |
| | QDQRIRWYLLSMG | |
| | SNENIHSIHFSGHVF | |
| | TVRKKEEYKMALY | |
| | NLYPGVFETVEMLP | |
| | SKAGIWRVECLIGE | |
| | HLHAGMSTLFLVY | |
| | SNKCQTPLGMASG | |
| | HIRDFQITASGQYG | |
| | QWAPKLARLHYSG | |
| | SINAWSTKEPFSWI | |
| | KVDLLAPMIIHGIK | |
| | TQGARQKFSSLYIS | |
| | QFIIMYSLDGKKW | |
| | QTYRGNSTGTLMV | |
| | FFGNVDSSGIKHNIF | |
| | NPPIIARYIRLHPTH | |
| | YSIRSTLRMELMGC | |
| | DLNSCSMPLGMES | |
| | KAISDAQITASSYFT | |
| | NMFATWSPSKARL | |
| | HLQGRSNAWRPQV | |
| | NNPKEWLQVDFQK | |
| | TMKVTGVTTQGVK | |
| | SLLTSMYVKEFLISS | |
| | SQDGHQWTLFFQN | |
| | GKVKVFQGNQDSF | |
| | TPVVNSLDPPLLTR | |
| | YLRIHPQSWVHQIA | |
| | LRMEVLGCEAQDL | |
| | YGAGSPGAETAEQ | |
| | KLISEEDLSPATG | |
| pBC0136 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNGAPG | GGAACAGAGGCATGACCGCCTTACTGAAGGTTTCTAGTTGTGA |
| | TSESATPESGPGSEPA | CAAGAACACTGGTGATTATTACGAGGACAGTTATGAAGATATT |
| | TSGSETPGTSESATPE | TCAGCATACTTGCTGAGTAAAAACAATGCCATTGAACCAAGA |
| | SGPGSEPATSGSETPG | AGCTTCTCTCAAAACGGCGCGCCAGGTACCTCAGAGTCTGCTA |
| | TSESATPESGPGTSTE | CCCCCGAGTCAGGGCCAGGATCAGAGCCAGCCACCTCCGGGT |
| | PSEGSAPGSPAGSPTS | CTGAGACACCCGGGACTTCCGAGAGTGCCACCCCTGAGTCCGG |
| | TEEGTSESATPESGPG | ACCCGGGTCCGAGCCCGCCACTTCCGGCTCCGAAACTCCCGGC |
| | SEPATSGSETPGTSES | ACAAGCGAGAGCGCTACCCCAGAGTCAGGACCAGGAACATCT |
| | ATPESGPGSPAGSPTS | ACAGAGCCCTCTGAAGGCTCCGCTCCAGGGTCCCCAGCCGGCA |
| | TEEGSPAGSPTSTEEG | GTCCCACTAGCACCGAGGAGGGAACCTCTGAAAGCGCCACAC |
| | TSTEPSEGSAPGTSES | CCGAATCAGGGCCAGGGTCTGAGCCTGCTACCAGCGGCAGCG |
| | ATPESGPGTSESATPE | AGACACCAGGCACCTCTGAGTCCGCCACACCAGAGTCCGGAC |
| | SGPGTSESATPESGPG | CCGGATCTCCCGCTGGGAGCCCCACCTCCACTGAGGAGGGATC |
| | SEPATSGSETPGSEPA | TCCTGCTGGCTCTCCAACATCTACTGAGGAAGGTACCTCAACC |
| | TSGSETPGSPAGSPTS | GAGCCATCCGAGGGATCAGCTCCCGGCACCTCAGAGTCGGCA |
| | TEEGTSTEPSEGSAPG | ACCCCGGAGTCTGGACCCGGAACTTCCGAAAGTGCCACACCA |
| | TSTEPSEGSAPGSEPA | GAGTCCGGTCCCGGGACTTCAGAATCAGCAACACCCGAGTCC |
| | TSGSETPGTSESATPE | GGCCCTGGGTCTGAACCCGCCACAAGTGGTAGTGAGACACCA |
| | SGPGTSTEPSEGSAPA | GGATCAGAACCTGCTACCTCAGGGTCAGAGACACCCGGATCTC |
| | SSPPVLKRHQREITRT | CGGCAGGCTCACCAACCTCCACTGAGGAGGGCACCAGCACAG |
| | TLQSDQEEIDYDDTIS | AACCAAGCGAGGGCTCCGCACCCGGAACAAGCACTGAACCCA |
| | VEMKKEDFDIYDEDE | GTGAGGGTTCAGCACCCGGCTCTGAGCCGGCCACAAGTGGCA |
| | NQSPRSFQKKTRHYFI | GTGAGACACCCGGCACTTCAGAGAGTGCCACCCCCGAGAGTG |
| | AAVERLWDYGMSSS | GCCCAGGCACTAGTACCGAGCCCTCTGAAGGCAGTGCGCCAG |
| | PHVLRNRAQSGSVPQ | CCTCGAGCCCACCAGTCTTGAAACGCCATCAACGGGAAATAA |
| | FKKVVFQEFTDGSFT | CTCGTACTACTCTTCAGTCAGATCAAGAGGAAATCGATTATGA |
| | QPLYRGELNEHLGLL | TGATACCATATCAGTTGAAATGAAGAAGGAAGATTTTGACATT |
| | GPYIRAEVEDNIMVT | TATGATGAGGATGAAAATCAGAGCCCCCGCAGCTTTCAAAAG |
| | FRNQASRPYSFYSSLI | AAAACACGACACTATTTTATTGCTGCAGTGGAGAGGCTCTGGG |
| | SYEEDQRQGAEPRKN | ATTATGGGATGAGTAGCTCCCCACATGTTCTAAGAAACAGGGC |
| | FVKPNETKTYFWKV | TCAGAGTGGCAGTGTCCCTCAGTTCAAGAAAGTTGTTTTCCAG |
| | QHHMAPTKDEFDCK | GAATTTACTGATGGCTCCTTTACTCAGCCCTTATACCGTGGAG |
| | AWAYFSDVDLEKDV | AACTAAATGAACATTTGGGACTCCTGGGGCCATATATAAGAGC |
| | HSGLIGPLLVCHTNTL | AGAAGTTGAAGATAATATCATGGTAACTTTCAGAAATCAGGCC |
| | NPAHGRQVTVQEFAL | TCTCGTCCCTATTCCTTCTATTCTAGCCTTATTTCTTATGAGGA |
| | FFTIFDETKSWYFTEN | AGATCAGAGGCAAGGAGCAGAACCTAGAAAAAACTTTGTCAA |
| | MERNCRAPCNIQMED | GCCTAATGAAACCAAAACTTACTTTTGGAAAGTGCAACATCAT |
| | PTFKENYRFHAINGYI | ATGGCACCCACTAAAGATGAGTTTGACTGCAAAGCCTGGGCTT |
| | MDTLPGLVMAQDQR | ATTTCTCTGATGTTGACCTGGAAAAAGATGTGCACTCAGGCCT |
| | IRWYLLSMGSNENIH | GATTGGACCCCTTCTGGTCTGCCACACTAACACACTGAACCCT |
| | SIHFSGHVFTVRKKEE | GCTCATGGGAGACAAGTGACAGTACAGGAATTTGCTCTGTTTT |
| | YKMALYNLYPGVFE | TCACCATCTTTGATGAGACCAAAAGCTGGTACTTCACTGAAAA |
| | TVEMLPSKAGIWRVE | TATGGAAAGAAACTGCAGGGCTCCCTGCAATATCCAGATGGA |
| | CLIGEHLHAGMSTLF | AGATCCCACTTTTAAAGAGAATTATCGCTTCCATGCAATCAAT |
| | LVYSNKCQTPLGMAS | GGCTACATAATGGATACACTACCTGGCTTAGTAATGGCTCAGG |
| | GHIRDFQITASGQYG | ATCAAAGGATTCGATGGTATCTGCTCAGCATGGGCAGCAATGA |
| | QWAPKLARLHYSGSI | AAACATCCATTCTATTCATTTCAGTGGACATGTGTTCACTGTAC |
| | NAWSTKEPFSWIKVD | GAAAAAAAGAGGAGTATAAAATGGCACTGTACAATCTCTATC |
| | LLAPMIIHGIKTQGAR | CAGGTGTTTTTGAGACAGTGGAAATGTTACCATCCAAAGCTGG |
| | QKFSSLYISQFIIMYSL | AATTTGGCGGGTGGAATGCCTTATTGGCGAGCATCTACATGCT |
| | DGKKWQTYRGNSTG | GGGATGAGCACACTTTTTCTGGTGTACAGCAATAAGTGTCAGA |
| | TLMVFFGNVDSSGIK | CTCCCCTGGGAATGGCTTCTGGACACATTAGAGATTTTCAGAT |
| | HNIFNPPIIARYIRLHP | TACAGCTTCAGGACAATATGGACAGTGGGCCCCAAAGCTGGC |
| | THYSIRSTLRMELMG | CAGACTTCATTATTCCGGATCAATCAATGCCTGGAGCACCAAG |
| | CDLNSCSMPLGMESK | GAGCCCTTTTCTTGGATCAAGGTGGATCTGTTGGCACCAATGA |
| | AISDAQITASSYFTNM | TTATTCACGGCATCAAGACCCAGGGTGCCCGTCAGAAGTTCTC |
| | FATWSPSKARLHLQG | CAGCCTCTACATCTCTCAGTTTATCATCATGTATAGTCTTGATG |
| | RSNAWRPQVNNPKE | GGAAGAAGTGGCAGACTTATCGAGGAAATTCCACTGGAACCT |
| | WLQVDFQKTMKVTG | TAATGGTCTTCTTTGGCAATGTGGATTCATCTGGGATAAAACA |
| | VTTQGVKSLLTSMYV | CAATATTTTTAACCCTCCAATTATTGCTCGATACATCCGTTTGC |
| | KEFLISSSQDGHQWT | ACCCAACTCATTATAGCATTCGCAGCACTCTTCGCATGGAGTT |
| | LFFQNGKVKVFQGN | GATGGGCTGTGATTTAAATAGTTGCAGCATGCCATTGGGAATG |
| | QDSFTPVVNSLDPPLL | GAGAGTAAAGCAATATCAGATGCACAGATTACTGCTTCATCCT |
| | TRYLRIHPQSWVHQI | ACTTTACCAATATGTTTGCCACCTGGTCTCCTTCAAAAGCTCGA |
| | ALRMEVLGCEAQDL | CTTCACCTCCAAGGGAGGAGTAATGCCTGGAGACCTCAGGTG |
| | YGAGSPGAETAEQKL | AATAATCCAAAAGAGTGGCTGCAAGTGGACTTCCAGAAGACA |
| | ISEEDLSPATG | ATGAAAGTCACAGGAGTAACTACTCAGGGAGTAAAATCTCTG |
| | | CTTACCAGCATGTATGTGAAGGAGTTCCTCATCTCCAGCAGTC |
| | | AAGATGGCCATCAGTGGACTCTCTTTTTTCAGAATGGCAAAGT |
| | | AAAGGTTTTTCAGGGAAATCAAGACTCCTTCACACCTGTGGTG |
| | | AACTCTCTAGACCCACCGTTACTGACTCGCTACCTTCGAATTC |
| | | ACCCCCAGAGTTGGGTGCACCAGATTGCCCTGAGGATGGAGG |
| | | TTCTGGGCTGCGAGGCACAGGACCTCTACGGCGCCGGATCACC |
| | | TGGGGCCGAAACGGCCGAACAAAAACTCATCTCAGAAGAGGA |
| | | TCTGTCACCTGCAACCGGTTGA |
| pBC0136-2 | MQIELSTCFFLCLL | atgcaaatagagctctccacctgcttctttctgtgccttttgcgattctgctttagtgccaccagaa |
| | RFCFSATRRYYLGA | gatactacctgggtgcagtggaactgtcatgggactatatgcaaagtgatctcggtgagctgc |
| | VELSWDYMQSDLG | ctgtggacgcaagatttcctcctagagtgccaaaatcttttccattcaacacctcagtcgtgtaca |
| | ELPVDARFPPRVPK | aaaagactctgtttgtagaattcacggatcaccttttcaacatcgctaagccaaggccaccctg |
| | SFPFNTSVVYKKTL | gatgggtctgctaggtcctaccatccaggctgaggtttatgatacagtggtcattacacttaaga |
| | FVEFTDHLFNIAKP | acatggcttcccatcctgtcagtcttcatgctgttggtgtatcctactggaaagcttctgagggag |
| | RPPWMGLLGPTIQA | ctgaatatgatgatcagaccagtcaaagggagaaagaagatgataaagtcttccctggtggaa |
| | EVYDTVVITLKNM | gccatacatatgtctggcaggtcctgaaagagaatggtccaatggcctctgacccactgtgcct |
| | ASHPVSLHAVGVS | tacctactcatatctttctcatgtggacctggtaaaagacttgaattcaggcctcattggagccct |
| | YWKASEGAEYDDQ | actagtatgtagagaagggagtctggccaaggaaaagacacagaccttgcacaaatttatact |
| | TSQREKEDDKVFPG | actttttgctgtatttgatgaagggaaaagttggcactcagaaacaaagaactccttgatgcagg |
| | GSHTYVWQVLKEN | atagggatgctgcatctgctcgggcctggcctaaaatgcacacagtcaatggttatgtaaaca |
| | GPMASDPLCLTYSY | ggtctctgccaggtctgattggatgccacaggaaatcagtctattggcatgtgattggaatggg |
| | LSHVDLVKDLNSG | caccactcctgaagtgcactcaatattcctcgaaggtcacacatttcttgtgaGGAACCA |
| | LIGALLVCREGSLA | TCGCCAGGCTAGCTTGGAAATCTCGCCAATAACTttccttactg |
| | KEKTQTLHKFILLF | ctcaaacactcttgatggaccttggacagtttctactgttttgtcatatctcttcccaccaacatgat |
| | AVFDEGKSWHSET | ggcatggaagcttatgtcaaagtagacagctgtccagaggaaccccaactacgaatgaaaaa |
| | KNSLMQDRDAASA | taatgaagaagcggaagactatgatgatgatcttactgattctgaaatggatgtggtcaggtttg |
| | RAWPKMHTVNGY | atgatgacaactctccttcctttatccaaattcgctcagttgccaagaagcatcctaaaacttggg |
| | VNRSLPGLIGCHRK | tacattacattgctgctgaagaggaggactgggactatgctcccttagtcctcgcccccgatga |
| | SVYWHVIGMGTTP | cagaagttataaaagtcaatatttgaacaatggccctcagcggattggtaggaagtacaaaaa |
| | EVHSIFLEGHTFLV | agtccgatttatggcatacacagatgaaacctttaagactcgtgaagctattcagcatgaatcag |
| | RNHRQASLEISPITF | gaatcttgggacctttactttatggggaagttggagacacactgttgattatatttaagaatcaag |
| | LTAQTLLMDLGQF | caagcagaccatataacatctaccctcacggaatcactgatgtccgtcctttgtattcaaggaga |
| | LLFCHISSHQHDGM | ttaccaaaaggtgtaaaacatttgaaggattttccaattctgccaggagaaatattcaaatataaa |
| | EAYVKVDSCPEEPQ | tggacagtgactgtagaagatgggccaactaaatcagatcctcggtgcctgacccgctattact |
| | LRMKNNEEAEDYD | ctagtttcgttaatatggagagagatctagcttcaggactcattggccctctcctcatctgctaca |
| | DDLTDSEMDVVRF | aagaatctgtagatcaaagaggaaaccagataatgtcagacaagaggaatgtcatcctgttttc |
| | DDDNSPSFIQIRSVA | tgtatttgatgagaaccgaagctggtacctcacagagaatatacaacgctttctccccaatcca |
| | KKHPKTWVHYIAA | gctggagtgcAGCTTGAGGATCCAGAGTTCcaagcctccaacatcatgca |
| | EEEDWDYAPLVLA | cagcatcaatggctatgtttttgatagtttgcagttgtcagtttgtttgcatgaggtggcatactggt |
| | PDDRSYKSQYLNN | acattctaagcattggagcacagactgacttcctttctgtcttcttctctggatataccttcaaaca |
| | GPQRIGRKYKKVRF | caaaatggtctatgaagacacactcaccctattcccattctcaggagaaactgtcttcatgtcga |
| | MAYTDETFKTREAI | tggaaaacccaggtctatggattctggggtgccacaactcagactttcggaacagaggcatga |
| | QHESGILGPLLYGE | ccgccttactgaaggtttctagttgtgacaagaacactggtgattattacgaggacagttatgaa |
| | VGDTLLIIFKNQAS | gatatttcagcatacttgctgagtaaaaacaatgccattgaaccaagaagcttctctGGCGC |
| | RPYNIYPHGITDVR | GCCAggtacctcagagtctgctaccCCCGAGTCAGGGCCAGGATCAG |
| | PLYSRRLPKGVKHL | AGCCAGCCACCTCCGGGTCTGAGACACCCGGGACTTCCG |
| | KDFPILPGEIFKYK | AGAGTGCCACCCCTGAGTCCGGACCCGGGTCCGAGCCC |
| | WTVTVEDGPTKSD | GCCACTTCCGGCTCCGAAACTCCCGGCACAAGCGAGAG |
| | PRCLTRYYSSFVNM | CGCTACCCCAGAGTCAGGACCAGGAACATCTACAGAGC |
| | ERDLASGLIGPLLIC | CCTCTGAAGGCTCCGCTCCAGGGTCCCCAGCCGGCAGTC |
| | YKESVDQRGNQIM | CCACTAGCACCGAGGAGGGAACCTCTGAAAGCGCCACA |
| | SDKRNVILFSVFDE | CCCGAATCAGGGCCAGGGTCTGAGCCTGCTACCAGCGGC |
| | NRSWYLTENIQRFL | AGCGAGACACCAGGCACCTCTGAGTCCGCCACACCAGA |
| | PNPAGVQLEDPEFQ | GTCCGGACCCGGATCTCCCGCTGGGAGCCCCACCTCCAC |
| | ASNIMHSINGYVFD | TGAGGAGGGATCTCCTGCTGGCTCTCCAACATCTACTGA |
| | SLQLSVCLHEVAY | GGAAGGTACCTCAACCGAGCCATCCGAGGGATCAGCTC |
| | WYILSIGAQTDFLS | CCGGCACCTCAGAGTCGGCAACCCCGGAGTCTGGACCC |
| | VFFSGYTFKHKMV | GGAACTTCCGAAAGTGCCACACCAGAGTCCGGTCCCGG |
| | YEDTLTLFPFSGET | GACTTCAGAATCAGCAACACCCGAGTCCGGCCCTGGGTC |
| | VFMSMENPGLWIL | TGAACCCGCCACAAGTGGTAGTGAGACACCAGGATCAG |
| | GCHNSDFRNRGMT | AACCTGCTACCTCAGGGTCAGAGACACCCGGATCTCCGG |
| | ALLKVSSCDKNTG | CAGGCTCACCAACCTCCACTGAGGAGGGCACCAGCACA |
| | DYYEDSYEDISAYL | GAACCAAGCGAGGGCTCCGCACCCGGAACAAGCACTGA |
| | LSKNNAIEPRSFSG | ACCCAGTGAGGGTTCAGCACCCGGCTCTGAGCCGGCCAC |
| | APGTSESATPESGP | AAGTGGCAGTGAGACACCCGGCACTTCAGAGAGTGCCA |
| | GSEPATSGSETPGT | CCCCCGAGAGTGGCCCAGGCACTAGTACCGAGCCCTCTg |
| | SESATPESGPGSEPA | aaggcagtgcgccaGCCTCGAGCcaaaacccaccagtcttgaaacgccatcaacgg |
| | TSGSETPGTSESATP | gaaataactcgtactacTCTTCAGTCAGATCAAGAGGAAATCGATT |
| | ESGPGTSTEPSEGS | ATGATGATACCATATCAGTTGAAATGAAgaaggaagattttgacat |
| | APGSPAGSPTSTEE | ttatgatgaggatgaaaatcagagcccccgcagctttcaaaagaaaacacgacactattttatt |
| | GTSESATPESGPGS | gctgcagtggagaggctctgggattatgggatgagtagctccccacatgttctaagaaacagg |
| | EPATSGSETPGTSES | gctcagagtggcagtgtccctcagttcaagaaagttgttttccaggaatttactgatggctccttt |
| | ATPESGPGSPAGSP | actcagcccttataccgtggagaactaaatgaacatttgggactcctggggccatatataagag |
| | TSTEEGSPAGSPTST | cagaagttgaagataatatcatggtaactttcagaaatcaggcctctcgtccctattccttctattc |
| | EEGTSTEPSEGSAP | tagccttatttcttatgaggaagatcagaggcaaggagcagaacctagaaaaaactttgtcaag |
| | GTSESATPESGPGT | cctaatgaaaccaaaacttacttttggaaagtgcaacatcatatggcacccactaaagatgagtt |
| | SESATPESGPGTSES | tgactgcaaagcctgggcttatttctctgatgttgacctggaaaaagatgtgcactcaggcctga |
| | ATPESGPGSEPATS | ttggaccccttctggtctgccacactaacacactgaaccctgctcatgggagacaagtgacag |
| | GSETPGSEPATSGS | tacaggaatttgctctgtttttcaccatctttgaTGAGACCAAAAGCTGGTACT |
| | ETPGSPAGSPTSTEE | TCactgaaaatatggaaagaaactgcagggctccctgcaatatccagatggaagatcccact |
| | GTSTEPSEGSAPGT | tttaaagagaattatcgcttccatgcaatcaatggctacataatggatacactacctggcttagta |
| | STEPSEGSAPGSEPA | atggctcaggatcaaaggattcgatggtatctgctcagcatgggcagcaatgaaaacatccatt |
| | TSGSETPGTSESATP | ctattcatttcagtggacatgtgttcactgtacgaaaaaaagaggagtataaaatggcactgtac |
| | ESGPGTSTEPSEGS | aatctctatccaggtgtttttgagacagtggaaatgttaccatccaaagctggaatttggcgggt |
| | APASSQNPPVLKRH | ggaatgccttattggcgagcatctacatgctgggatgagcacactttttctggtgtACAGC |
| | QREITRTTLQSDQE | AATAAGTGTCAGACTCCcctgggaatggcttctggacacattagagattttcag |
| | EIDYDDTISVEMKK | attacagcttcaggacaatatggacagtgggccccaaagctggccagacttcattattccggat |
| | EDFDIYDEDENQSP | caatcaatgcctggagcaccaaggagcccttttcttggatcaaggtggatctgttggcaccaat |
| | RSFQKKTRHYFIAA | gattattcacggcatcaagacccagggtgcccgtcagaagttctccagcctctacatctctcag |
| | VERLWDYGMSSSP | tttatcatcatgtatagtcttgatgggaagaagtggcagacttatcgaggaaattccactggaac |
| | HVLRNRAQSGSVP | cttaatggtcttctttggcaatgtggattcatctgggataaaacacaatatttttaaccctccaatta |
| | QFKKVVFQEFTDGS | ttgctcgatacatccgtttgcacccaactcattatagcattcgcagcactcttcgcatggagttga |
| | FTQPLYRGELNEHL | tgggctgtgatttaaatagttgcagcatgccattgggaatggagagtaaagcaatatcagatgc |
| | GLLGPYIRAEVEDN | acagattactgcttcatcctactttaccaatatgtttgccacctggtctccttcaaaagctcgacttc |
| | IMVTFRNQASRPYS | acctccaagggaggagtaatgcctggagacctcaggtgaataatccaaaagagtggctgca |
| | FYSSLISYEEDQRQ | agtggacttccagaagacaatgaaagtcacaggagtaactactcagggagtaaaatctctgct |
| | GAEPRKNFVKPNET | taccagcatgtatgtgaaggagttcctcatctccagcagtcaagatggccatcagtggactctc |
| | KTYFWKVQHHMA | ttttttcagaatggcaaagtaaaggtttttcagggaaatcaagactccttcacacctgtggtgaac |
| | PTKDEFDCKAWAY | tctctagacccaccgttactgactcgctaccttcgaattcacccccagagttgggtgcaccaga |
| | FSDVDLEKDVHSG | ttgccctgaggatggaggttctgggctgcgaggcacaggacctctacggcgccggatcacct |
| | LIGPLLVCHTNTLN | ggggccgaaacggccgaacaaaaactcatctcagaagaggatctgtcacctgcaaccggtt |
| | PAHGRQVTVQEFA | ga |
| | LFFTIFDETKSWYF | |
| | TENMERNCRAPCNI | |
| | QMEDPTFKENYRF | |
| | HAINGYIMDTLPGL | |
| | VMAQDQRIRWYLL | |
| | SMGSNENIHSIHFS | |
| | GHVFTVRKKEEYK | |
| | MALYNLYPGVFET | |
| | VEMLPSKAGIWRV | |
| | ECLIGEHLHAGMST | |
| | LFLVYSNKCQTPLG | |
| | MASGHIRDFQITAS | |
| | GQYGQWAPKLARL | |
| | HYSGSINAWSTKEP | |
| | FSWIKVDLLAPMII | |
| | HGIKTQGARQKFSS | |
| | LYISQFIIMYSLDGK | |
| | KWQTYRGNSTGTL | |
| | MVFFGNVDSSGIKH | |
| | NIFNPPIIARYIRLHP | |
| | THYSIRSTLRMELM | |
| | GCDLNSCSMPLGM | |
| | ESKAISDAQITASSY | |
| | FTNMFATWSPSKA | |
| | RLHLQGRSNAWRP | |
| | QVNNPKEWLQVDF | |
| | QKTMKVTGVTTQG | |
| | VKSLLTSMYVKEFL | |
| | ISSSQDGHQWTLFF | |
| | QNGKVKVFQGNQD | |
| | SFTPVVNSLDPPLL | |
| | TRYLRIHPQSWVH | |
| | QIALRMEVLGCEA | |
| | QDLYGAGSPGAET | |
| | AEQKLISEEDLSPA | |
| | TG | |
| pBC0137 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNGAPG | GGAACAGAGGCATGACCGCCTTACTGAAGGTTTCTAGTTGTGA |
| | TSESATPESGPGSEPA | CAAGAACACTGGTGATTATTACGAGGACAGTTATGAAGATATT |
| | TSGSETPGTSESATPE | TCAGCATACTTGCTGAGTAAAAACAATGCCATTGAACCAAGA |
| | SGPGSEPATSGSETPG | AGCTTCTCTCAAAACGGCGCGCCAGGTACCTCAGAGTCTGCTA |
| | TSESATPESGPGTSTE | CCCCCGAGTCAGGGCCAGGATCAGAGCCAGCCACCTCCGGGT |
| | PSEGSAPGSPAGSPTS | CTGAGACACCCGGGACTTCCGAGAGTGCCACCCCTGAGTCCGG |
| | TEEGTSESATPESGPG | ACCCGGGTCCGAGCCCGCCACTTCCGGCTCCGAAACTCCCGGC |
| | SEPATSGSETPGTSES | ACAAGCGAGAGCGCTACCCCAGAGTCAGGACCAGGAACATCT |
| | ATPESGPGSPAGSPTS | ACAGAGCCCTCTGAAGGCTCCGCTCCAGGGTCCCCAGCCGGCA |
| | TEEGSPAGSPTSTEEG | GTCCCACTAGCACCGAGGAGGGAACCTCTGAAAGCGCCACAC |
| | TSTEPSEGSAPGTSES | CCGAATCAGGGCCAGGGTCTGAGCCTGCTACCAGCGGCAGCG |
| | ATPESGPGTSESATPE | AGACACCAGGCACCTCTGAGTCCGCCACACCAGAGTCCGGAC |
| | SGPGTSESATPESGPG | CCGGATCTCCCGCTGGGAGCCCCACCTCCACTGAGGAGGGATC |
| | SEPATSGSETPGSEPA | TCCTGCTGGCTCTCCAACATCTACTGAGGAAGGTACCTCAACC |
| | TSGSETPGSPAGSPTS | GAGCCATCCGAGGGATCAGCTCCCGGCACCTCAGAGTCGGCA |
| | TEEGTSTEPSEGSAPG | ACCCCGGAGTCTGGACCCGGAACTTCCGAAAGTGCCACACCA |
| | TSTEPSEGSAPGSEPA | GAGTCCGGTCCCGGGACTTCAGAATCAGCAACACCCGAGTCC |
| | TSGSETPGTSESATPE | GGCCCTGGGTCTGAACCCGCCACAAGTGGTAGTGAGACACCA |
| | SGPGTSTEPSEGSAPA | GGATCAGAACCTGCTACCTCAGGGTCAGAGACACCCGGATCTC |
| | SSPPVLKRHQAEITRT | CGGCAGGCTCACCAACCTCCACTGAGGAGGGCACCAGCACAG |
| | TLQSDQEEIDYDDTIS | AACCAAGCGAGGGCTCCGCACCCGGAACAAGCACTGAACCCA |
| | VEMKKEDFDIYDEDE | GTGAGGGTTCAGCACCCGGCTCTGAGCCGGCCACAAGTGGCA |
| | NQSPRSFQKKTRHYFI | GTGAGACACCCGGCACTTCAGAGAGTGCCACCCCCGAGAGTG |
| | AAVERLWDYGMSSS | GCCCAGGCACTAGTACCGAGCCCTCTGAAGGCAGTGCGCCAG |
| | PHVLRNRAQSGSVPQ | CCTCGAGCCCACCAGTCTTGAAACGCCATCAAGCTGAAATAAC |
| | FKKVVFQEFTDGSFT | TCGTACTACTCTTCAGTCAGATCAAGAGGAAATCGATTATGAT |
| | QPLYRGELNEHLGLL | GATACCATATCAGTTGAAATGAAGAAGGAAGATTTTGACATTT |
| | GPYIRAEVEDNIMVT | ATGATGAGGATGAAAATCAGAGCCCCCGCAGCTTTCAAAAGA |
| | FRNQASRPYSFYSSLI | AAACACGACACTATTTTATTGCTGCAGTGGAGAGGCTCTGGGA |
| | SYEEDQRQGAEPRKN | TTATGGGATGAGTAGCTCCCCACATGTTCTAAGAAACAGGGCT |
| | FVKPNETKTYFWKV | CAGAGTGGCAGTGTCCCTCAGTTCAAGAAAGTTGTTTTCCAGG |
| | QHHMAPTKDEFDCK | AATTTACTGATGGCTCCTTTACTCAGCCCTTATACCGTGGAGA |
| | AWAYFSDVDLEKDV | ACTAAATGAACATTTGGGACTCCTGGGGCCATATATAAGAGCA |
| | HSGLIGPLLVCHTNTL | GAAGTTGAAGATAATATCATGGTAACTTTCAGAAATCAGGCCT |
| | NPAHGRQVTVQEFAL | CTCGTCCCTATTCCTTCTATTCTAGCCTTATTTCTTATGAGGAA |
| | FFTIFDETKSWYFTEN | GATCAGAGGCAAGGAGCAGAACCTAGAAAAAACTTTGTCAAG |
| | MERNCRAPCNIQMED | CCTAATGAAACCAAAACTTACTTTTGGAAAGTGCAACATCATA |
| | PTFKENYRFHAINGYI | TGGCACCCACTAAAGATGAGTTTGACTGCAAAGCCTGGGCTTA |
| | MDTLPGLVMAQDQR | TTTCTCTGATGTTGACCTGGAAAAAGATGTGCACTCAGGCCTG |
| | IRWYLLSMGSNENIH | ATTGGACCCCTTCTGGTCTGCCACACTAACACACTGAACCCTG |
| | SIHFSGHVFTVRKKEE | CTCATGGGAGACAAGTGACAGTACAGGAATTTGCTCTGTTTTT |
| | YKMALYNLYPGVFE | CACCATCTTTGATGAGACCAAAAGCTGGTACTTCACTGAAAAT |
| | TVEMLPSKAGIWRVE | ATGGAAAGAAACTGCAGGGCTCCCTGCAATATCCAGATGGAA |
| | CLIGEHLHAGMSTLF | GATCCCACTTTTAAAGAGAATTATCGCTTCCATGCAATCAATG |
| | LVYSNKCQTPLGMAS | GCTACATAATGGATACACTACCTGGCTTAGTAATGGCTCAGGA |
| | GHIRDFQITASGQYG | TCAAAGGATTCGATGGTATCTGCTCAGCATGGGCAGCAATGAA |
| | QWAPKLARLHYSGSI | AACATCCATTCTATTCATTTCAGTGGACATGTGTTCACTGTACG |
| | NAWSTKEPFSWIKVD | AAAAAAAGAGGAGTATAAAATGGCACTGTACAATCTCTATCC |
| | LLAPMIIHGIKTQGAR | AGGTGTTTTTGAGACAGTGGAAATGTTACCATCCAAAGCTGGA |
| | QKFSSLYISQFIIMYSL | ATTTGGCGGGTGGAATGCCTTATTGGCGAGCATCTACATGCTG |
| | DGKKWQTYRGNSTG | GGATGAGCACACTTTTTCTGGTGTACAGCAATAAGTGTCAGAC |
| | TLMVFFGNVDSSGIK | TCCCCTGGGAATGGCTTCTGGACACATTAGAGATTTTCAGATT |
| | HNIFNPPIIARYIRLHP | ACAGCTTCAGGACAATATGGACAGTGGGCCCCAAAGCTGGCC |
| | THYSIRSTLRMELMG | AGACTTCATTATTCCGGATCAATCAATGCCTGGAGCACCAAGG |
| | CDLNSCSMPLGMESK | AGCCCTTTTCTTGGATCAAGGTGGATCTGTTGGCACCAATGAT |
| | AISDAQITASSYFTNM | TATTCACGGCATCAAGACCCAGGGTGCCCGTCAGAAGTTCTCC |
| | FATWSPSKARLHLQG | AGCCTCTACATCTCTCAGTTTATCATCATGTATAGTCTTGATGG |
| | RSNAWRPQVNNPKE | GAAGAAGTGGCAGACTTATCGAGGAAATTCCACTGGAACCTT |
| | WLQVDFQKTMKVTG | AATGGTCTTCTTTGGCAATGTGGATTCATCTGGGATAAAACAC |
| | VTTQGVKSLLTSMYV | AATATTTTTAACCCTCCAATTATTGCTCGATACATCCGTTTGCA |
| | KEFLISSSQDGHQWT | CCCAACTCATTATAGCATTCGCAGCACTCTTCGCATGGAGTTG |
| | LFFQNGKVKVFQGN | ATGGGCTGTGATTTAAATAGTTGCAGCATGCCATTGGGAATGG |
| | QDSFTPVVNSLDPPLL | AGAGTAAAGCAATATCAGATGCACAGATTACTGCTTCATCCTA |
| | TRYLRIHPQSWVHQI | CTTTACCAATATGTTTGCCACCTGGTCTCCTTCAAAAGCTCGAC |
| | ALRMEVLGCEAQDL | TTCACCTCCAAGGGAGGAGTAATGCCTGGAGACCTCAGGTGA |
| | YGAGSPGAETAEQKL | ATAATCCAAAAGAGTGGCTGCAAGTGGACTTCCAGAAGACAA |
| | ISEEDLSPATG | TGAAAGTCACAGGAGTAACTACTCAGGGAGTAAAATCTCTGCT |
| | | TACCAGCATGTATGTGAAGGAGTTCCTCATCTCCAGCAGTCAA |
| | | GATGGCCATCAGTGGACTCTCTTTTTTCAGAATGGCAAAGTAA |
| | | AGGTTTTTCAGGGAAATCAAGACTCCTTCACACCTGTGGTGAA |
| | | CTCTCTAGACCCACCGTTACTGACTCGCTACCTTCGAATTCACC |
| | | CCCAGAGTTGGGTGCACCAGATTGCCCTGAGGATGGAGGTTCT |
| | | GGGCTGCGAGGCACAGGACCTCTACGGCGCCGGATCACCTGG |
| | | GGCCGAAACGGCCGAACAAAAACTCATCTCAGAAGAGGATCT |
| | | GTCACCTGCAACCGGTTGA |
| pBC0137-2 | MQIELSTCFFLCLL | atgcaaatagagctctccacctgcttctttctgtgccttttgcgattctgctttagtgccaccagaa |
| | RFCFSATRRYYLGA | gatactacctgggtgcagtggaactgtcatgggactatatgcaaagtgatctcggtgagctgc |
| | VELSWDYMQSDLG | ctgtggacgcaagatttcctcctagagtgccaaaatcttttccattcaacacctcagtcgtgtaca |
| | ELPVDARFPPRVPK | aaaagactctgtttgtagaattcacggatcaccttttcaacatcgctaagccaaggccaccctg |
| | SFPFNTSVVYKKTL | gatgggtctgctaggtcctaccatccaggctgaggtttatgatacagtggtcattacacttaaga |
| | FVEFTDHLFNIAKP | acatggcttcccatcctgtcagtcttcatgctgttggtgtatcctactggaaagcttctgagggag |
| | RPPWMGLLGPTIQA | ctgaatatgatgatcagaccagtcaaagggagaaagaagatgataaagtcttccctggtggaa |
| | EVYDTVVITLKNM | gccatacatatgtctggcaggtcctgaaagagaatggtccaatggcctctgacccactgtgcct |
| | ASHPVSLHAVGVS | tacctactcatatctttctcatgtggacctggtaaaagacttgaattcaggcctcattggagccct |
| | YWKASEGAEYDDQ | actagtatgtagagaagggagtctggccaaggaaaagacacagaccttgcacaaatttatact |
| | TSQREKEDDKVFPG | actttttgctgtatttgatgaagggaaaagttggcactcagaaacaaagaactccttgatgcagg |
| | GSHTYVWQVLKEN | atagggatgctgcatctgctcgggcctggcctaaaatgcacacagtcaatggttatgtaaaca |
| | GPMASDPLCLTYSY | ggtctctgccaggtctgattggatgccacaggaaatcagtctattggcatgtgattggaatggg |
| | LSHVDLVKDLNSG | caccactcctgaagtgcactcaatattcctcgaaggtcacacatttcttgtgaGGAACCA |
| | LIGALLVCREGSLA | TCGCCAGGCTAGCTTGGAAATCTCGCCAATAACTttccttactg |
| | KEKTQTLHKFILLF | ctcaaacactcttgatggaccttggacagtttctactgttttgtcatatctcttcccaccaacatgat |
| | AVFDEGKSWHSET | ggcatggaagcttatgtcaaagtagacagctgtccagaggaaccccaactacgaatgaaaaa |
| | KNSLMQDRDAASA | taatgaagaagcggaagactatgatgatgatcttactgattctgaaatggatgtggtcaggtttg |
| | RAWPKMHTVNGY | atgatgacaactctccttcctttatccaaattcgctcagttgccaagaagcatcctaaaacttggg |
| | VNRSLPGLIGCHRK | tacattacattgctgctgaagaggaggactgggactatgctcccttagtcctcgcccccgatga |
| | SVYWHVIGMGTTP | cagaagttataaaagtcaatatttgaacaatggccctcagcggattggtaggaagtacaaaaa |
| | EVHSIFLEGHTFLV | agtccgatttatggcatacacagatgaaacctttaagactcgtgaagctattcagcatgaatcag |
| | RNHRQASLEISPITF | gaatcttgggacctttactttatggggaagttggagacacactgttgattatatttaagaatcaag |
| | LTAQTLLMDLGQF | caagcagaccatataacatctaccctcacggaatcactgatgtccgtcctttgtattcaaggaga |
| | LLFCHISSHQHDGM | ttaccaaaaggtgtaaaacatttgaaggattttccaattctgccaggagaaatattcaaatataaa |
| | EAYVKVDSCPEEPQ | tggacagtgactgtagaagatgggccaactaaatcagatcctcggtgcctgacccgctattact |
| | LRMKNNEEAEDYD | ctagtttcgttaatatggagagagatctagcttcaggactcattggccctctcctcatctgctaca |
| | DDLTDSEMDVVRF | aagaatctgtagatcaaagaggaaaccagataatgtcagacaagaggaatgtcatcctgttttc |
| | DDDNSPSFIQIRSVA | tgtatttgatgagaaccgaagctggtacctcacagagaatatacaacgctttctccccaatcca |
| | KKHPKTWVHYIAA | gctggagtgcAGCTTGAGGATCCAGAGTTCcaagcctccaacatcatgca |
| | EEEDWDYAPLVLA | cagcatcaatggctatgtttttgatagtttgcagttgtcagtttgtttgcatgaggtggcatactggt |
| | PDDRSYKSQYLNN | acattctaagcattggagcacagactgacttcctttctgtcttcttctctggatataccttcaaaca |
| | GPQRIGRKYKKVRF | caaaatggtctatgaagacacactcaccctattcccattctcaggagaaactgtcttcatgtcga |
| | MAYTDETFKTREAI | tggaaaacccaggtctatggattctggggtgccacaactcagactttcggaacagaggcatga |
| | QHESGILGPLLYGE | ccgccttactgaaggtttctagttgtgacaagaacactggtgattattacgaggacagttatgaa |
| | VGDTLLIIFKNQAS | gatatttcagcatacttgctgagtaaaaacaatgccattgaaccaagaagcttctctGGCGC |
| | RPYNIYPHGITDVR | GCCAggtacctcagagtctgctaccCCCGAGTCAGGGCCAGGATCAG |
| | PLYSRRLPKGVKHL | AGCCAGCCACCTCCGGGTCTGAGACACCCGGGACTTCCG |
| | KDFPILPGEIFKYK | AGAGTGCCACCCCTGAGTCCGGACCCGGGTCCGAGCCC |
| | WTVTVEDGPTKSD | GCCACTTCCGGCTCCGAAACTCCCGGCACAAGCGAGAG |
| | PRCLTRYYSSFVNM | CGCTACCCCAGAGTCAGGACCAGGAACATCTACAGAGC |
| | ERDLASGLIGPLLIC | CCTCTGAAGGCTCCGCTCCAGGGTCCCCAGCCGGCAGTC |
| | YKESVDQRGNQIM | CCACTAGCACCGAGGAGGGAACCTCTGAAAGCGCCACA |
| | SDKRNVILFSVFDE | CCCGAATCAGGGCCAGGGTCTGAGCCTGCTACCAGCGGC |
| | NRSWYLTENIQRFL | AGCGAGACACCAGGCACCTCTGAGTCCGCCACACCAGA |
| | PNPAGVQLEDPEFQ | GTCCGGACCCGGATCTCCCGCTGGGAGCCCCACCTCCAC |
| | ASNIMHSINGYVFD | TGAGGAGGGATCTCCTGCTGGCTCTCCAACATCTACTGA |
| | SLQLSVCLHEVAY | GGAAGGTACCTCAACCGAGCCATCCGAGGGATCAGCTC |
| | WYILSIGAQTDFLS | CCGGCACCTCAGAGTCGGCAACCCCGGAGTCTGGACCC |
| | VFFSGYTFKHKMV | GGAACTTCCGAAAGTGCCACACCAGAGTCCGGTCCCGG |
| | YEDTLTLFPFSGET | GACTTCAGAATCAGCAACACCCGAGTCCGGCCCTGGGTC |
| | VFMSMENPGLWIL | TGAACCCGCCACAAGTGGTAGTGAGACACCAGGATCAG |
| | GCHNSDFRNRGMT | AACCTGCTACCTCAGGGTCAGAGACACCCGGATCTCCGG |
| | ALLKVSSCDKNTG | CAGGCTCACCAACCTCCACTGAGGAGGGCACCAGCACA |
| | DYYEDSYEDISAYL | GAACCAAGCGAGGGCTCCGCACCCGGAACAAGCACTGA |
| | LSKNNAIEPRSFSG | ACCCAGTGAGGGTTCAGCACCCGGCTCTGAGCCGGCCAC |
| | APGTSESATPESGP | AAGTGGCAGTGAGACACCCGGCACTTCAGAGAGTGCCA |
| | GSEPATSGSETPGT | CCCCCGAGAGTGGCCCAGGCACTAGTACCGAGCCCTCTg |
| | SESATPESGPGSEPA | aaggcagtgcgccaGCCTCGAGCcaaaacccaccagtcttgaaacgccatcaaGC |
| | TSGSETPGTSESATP | TgaaataactcgtactacTCTTCAGTCAGATCAAGAGGAAATCGAT |
| | ESGPGTSTEPSEGS | TATGATGATACCATATCAGTTGAAATGAAgaaggaagattttgac |
| | APGSPAGSPTSTEE | atttatgatgaggatgaaaatcagagcccccgcagctttcaaaagaaaacacgacactattttat |
| | GTSESATPESGPGS | tgctgcagtggagaggctctgggattatgggatgagtagctccccacatgttctaagaaacag |
| | EPATSGSETPGTSES | ggctcagagtggcagtgtccctcagttcaagaaagttgttttccaggaatttactgatggctcctt |
| | ATPESGPGSPAGSP | tactcagcccttataccgtggagaactaaatgaacatttgggactcctggggccatatataaga |
| | TSTEEGSPAGSPTST | gcagaagttgaagataatatcatggtaactttcagaaatcaggcctctcgtccctattccttctatt |
| | EEGTSTEPSEGSAP | ctagccttatttcttatgaggaagatcagaggcaaggagcagaacctagaaaaaactttgtcaa |
| | GTSESATPESGPGT | gcctaatgaaaccaaaacttacttttggaaagtgcaacatcatatggcacccactaaagatgag |
| | SESATPESGPGTSES | tttgactgcaaagcctgggcttatttctctgatgttgacctggaaaaagatgtgcactcaggcct |
| | ATPESGPGSEPATS | gattggaccccttctggtctgccacactaacacactgaaccctgctcatgggagacaagtgac |
| | GSETPGSEPATSGS | agtacaggaatttgctctgtttttcaccatctttgaTGAGACCAAAAGCTGGTA |
| | ETPGSPAGSPTSTEE | CTTCactgaaaatatggaaagaaactgcagggctccctgcaatatccagatggaagatcc |
| | GTSTEPSEGSAPGT | cacttttaaagagaattatcgcttccatgcaatcaatggctacataatggatacactacctggctt |
| | STEPSEGSAPGSEPA | agtaatggctcaggatcaaaggattcgatggtatctgctcagcatgggcagcaatgaaaacat |
| | TSGSETPGTSESATP | ccattctattcatttcagtggacatgtgttcactgtacgaaaaaaagaggagtataaaatggcac |
| | ESGPGTSTEPSEGS | tgtacaatctctatccaggtgtttttgagacagtggaaatgttaccatccaaagctggaatttggc |
| | APASSQNPPVLKRH | gggtggaatgccttattggcgagcatctacatgctgggatgagcacactttttctggtgtACA |
| | QAEITRTTLQSDQE | GCAATAAGTGTCAGACTCCcctgggaatggcttctggacacattagagatttt |
| | EIDYDDTISVEMKK | cagattacagcttcaggacaatatggacagtgggccccaaagctggccagacttcattattcc |
| | EDFDIYDEDENQSP | ggatcaatcaatgcctggagcaccaaggagcccttttcttggatcaaggtggatctgttggcac |
| | RSFQKKTRHYFIAA | caatgattattcacggcatcaagacccagggtgcccgtcagaagttctccagcctctacatctc |
| | VERLWDYGMSSSP | tcagtttatcatcatgtatagtcttgatgggaagaagtggcagacttatcgaggaaattccactg |
| | HVLRNRAQSGSVP | gaaccttaatggtcttctttggcaatgtggattcatctgggataaaacacaatatttttaaccctcc |
| | QFKKVVFQEFTDGS | aattattgctcgatacatccgtttgcacccaactcattatagcattcgcagcactcttcgcatgga |
| | FTQPLYRGELNEHL | gttgatgggctgtgatttaaatagttgcagcatgccattgggaatggagagtaaagcaatatca |
| | GLLGPYIRAEVEDN | gatgcacagattactgcttcatcctactttaccaatatgtttgccacctggtctccttcaaaagctc |
| | IMVTFRNQASRPYS | gacttcacctccaagggaggagtaatgcctggagacctcaggtgaataatccaaaagagtgg |
| | FYSSLISYEEDQRQ | ctgcaagtggacttccagaagacaatgaaagtcacaggagtaactactcagggagtaaaatct |
| | GAEPRKNFVKPNET | ctgcttaccagcatgtatgtgaaggagttcctcatctccagcagtcaagatggccatcagtgga |
| | KTYFWKVQHHMA | ctctcttttttcagaatggcaaagtaaaggtttttcagggaaatcaagactccttcacacctgtggt |
| | PTKDEFDCKAWAY | gaactctctagacccaccgttactgactcgctaccttcgaattcacccccagagttgggtgcac |
| | FSDVDLEKDVHSG | cagattgccctgaggatggaggttctgggctgcgaggcacaggacctctacggcgccggat |
| | LIGPLLVCHTNTLN | cacctggggccgaaacggccgaacaaaaactcatctcagaagaggatctgtcacctgcaac |
| | PAHGRQVTVQEFA | cggttga |
| | LFFTIFDETKSWYF | |
| | TENMERNCRAPCNI | |
| | QMEDPTFKENYRF | |
| | HAINGYIMDTLPGL | |
| | VMAQDQRIRWYLL | |
| | SMGSNENIHSIHFS | |
| | GHVFTVRKKEEYK | |
| | MALYNLYPGVFET | |
| | VEMLPSKAGIWRV | |
| | ECLIGEHLHAGMST | |
| | LFLVYSNKCQTPLG | |
| | MASGHIRDFQITAS | |
| | GQYGQWAPKLARL | |
| | HYSGSINAWSTKEP | |
| | FSWIKVDLLAPMII | |
| | HGIKTQGARQKFSS | |
| | LYISQFIIMYSLDGK | |
| | KWQTYRGNSTGTL | |
| | MVFFGNVDSSGIKH | |
| | NIFNPPIIARYIRLHP | |
| | THYSIRSTLRMELM | |
| | GCDLNSCSMPLGM | |
| | ESKAISDAQITASSY | |
| | FTNMFATWSPSKA | |
| | RLHLQGRSNAWRP | |
| | QVNNPKEWLQVDF | |
| | QKTMKVTGVTTQG | |
| | VKSLLTSMYVKEFL | |
| | ISSSQDGHQWTLFF | |
| | QNGKVKVFQGNQD | |
| | SFTPVVNSLDPPLL | |
| | TRYLRIHPQSWVH | |
| | QIALRMEVLGCEA | |
| | QDLYGAGSPGAET | |
| | AEQKLISEEDLSPA | |
| | TG | |
| pBC0138 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQGAPGSPAGSP | GACATTTATGATGAGGATGAAAATCAGAGCCCCCGCAGCTTTC |
| | TSTEEGTSESATPESG | AAAAGAAAACACGACACTATTTTATTGCTGCAGTGGAGAGGC |
| | PGSEPATSGSETPASS | TCTGGGATTATGGGATGAGTAGCTCCCCACATGTTCTAAGAAA |
| | RAQSGSVPQFKKVVF | CAGGGCTCAGGGCGCGCCAGGTTCTCCTGCTGGCTCCCCCACC |
| | QEFTDGSFTQPLYRG | TCAACAGAAGAGGGGACAAGCGAAAGCGCTACGCCTGAGAGT |
| | ELNEHLGLLGPYIRAE | GGCCCTGGCTCTGAGCCAGCCACCTCCGGCTCTGAAACCCCTG |
| | VEDNIMVTFRNQASR | CCTCGAGCAGGGCTCAGAGTGGCAGTGTCCCTCAGTTCAAGAA |
| | PYSFYSSLISYEEDQR | AGTTGTTTTCCAGGAATTTACTGATGGCTCCTTTACTCAGCCCT |
| | QGAEPRKNFVKPNET | TATACCGTGGAGAACTAAATGAACATTTGGGACTCCTGGGGCC |
| | KTYFWKVQHHMAPT | ATATATAAGAGCAGAAGTTGAAGATAATATCATGGTAACTTTC |
| | KDEFDCKAWAYFSD | AGAAATCAGGCCTCTCGTCCCTATTCCTTCTATTCTAGCCTTAT |
| | VDLEKDVHSGLIGPL | TTCTTATGAGGAAGATCAGAGGCAAGGAGCAGAACCTAGAAA |
| | LVCHTNTLNPAHGRQ | AAACTTTGTCAAGCCTAATGAAACCAAAACTTACTTTTGGAAA |
| | VTVQEFALFFTIFDET | GTGCAACATCATATGGCACCCACTAAAGATGAGTTTGACTGCA |
| | KSWYFTENMERNCR | AAGCCTGGGCTTATTTCTCTGATGTTGACCTGGAAAAAGATGT |
| | APCNIQMEDPTFKEN | GCACTCAGGCCTGATTGGACCCCTTCTGGTCTGCCACACTAAC |
| | YRFHAINGYIMDTLP | ACACTGAACCCTGCTCATGGGAGACAAGTGACAGTACAGGAA |
| | GLVMAQDQRIRWYL | TTTGCTCTGTTTTTCACCATCTTTGATGAGACCAAAAGCTGGTA |
| | LSMGSNENIHSIHFSG | CTTCACTGAAAATATGGAAAGAAACTGCAGGGCTCCCTGCAAT |
| | HVFTVRKKEEYKMA | ATCCAGATGGAAGATCCCACTTTTAAAGAGAATTATCGCTTCC |
| | LYNLYPGVFETVEML | ATGCAATCAATGGCTACATAATGGATACACTACCTGGCTTAGT |
| | PSKAGIWRVECLIGE | AATGGCTCAGGATCAAAGGATTCGATGGTATCTGCTCAGCATG |
| | HLHAGMSTLFLVYSN | GGCAGCAATGAAAACATCCATTCTATTCATTTCAGTGGACATG |
| | KCQTPLGMASGHIRD | TGTTCACTGTACGAAAAAAAGAGGAGTATAAAATGGCACTGT |
| | FQITASGQYGQWARK | ACAATCTCTATCCAGGTGTTTTTGAGACAGTGGAAATGTTACC |
| | LARLHYSGSINAWST | ATCCAAAGCTGGAATTTGGCGGGTGGAATGCCTTATTGGCGAG |
| | KEPFSWIKVDLLAPMI | CATCTACATGCTGGGATGAGCACACTTTTTCTGGTGTACAGCA |
| | IHGIKTQGARQKFSSL | ATAAGTGTCAGACTCCCCTGGGAATGGCTTCTGGACACATTAG |
| | YISQFIIMYSLDGKKW | AGATTTTCAGATTACAGCTTCAGGACAATATGGACAGTGGGCC |
| | QTYRGNSTGTLMVFF | CCAAAGCTGGCCAGACTTCATTATTCCGGATCAATCAATGCCT |
| | GNVDSSGIKHNIFNPP | GGAGCACCAAGGAGCCCTTTTCTTGGATCAAGGTGGATCTGTT |
| | IIARYIRLHPTHYSIRS | GGCACCAATGATTATTCACGGCATCAAGACCCAGGGTGCCCGT |
| | TLRMELMGCDLNSCS | CAGAAGTTCTCCAGCCTCTACATCTCTCAGTTTATCATCATGTA |
| | MPLGMESKAISDAQI | TAGTCTTGATGGGAAGAAGTGGCAGACTTATCGAGGAAATTCC |
| | TASSYFTNMFATWSP | ACTGGAACCTTAATGGTCTTCTTTGGCAATGTGGATTCATCTG |
| | SKARLHLQGRSNAW | GGATAAAACACAATATTTTTAACCCTCCAATTATTGCTCGATA |
| | RPQVNNPKEWLQVD | CATCCGTTTGCACCCAACTCATTATAGCATTCGCAGCACTCTTC |
| | FQKTMKVTGVTTQG | GCATGGAGTTGATGGGCTGTGATTTAAATAGTTGCAGCATGCC |
| | VKSLLTSMYVKEFLIS | ATTGGGAATGGAGAGTAAAGCAATATCAGATGCACAGATTAC |
| | SSQDGHQWTLFFQNG | TGCTTCATCCTACTTTACCAATATGTTTGCCACCTGGTCTCCTT |
| | KVKVFQGNQDSFTPV | CAAAAGCTCGACTTCACCTCCAAGGGAGGAGTAATGCCTGGA |
| | VNSLDPPLLTRYLRIH | GACCTCAGGTGAATAATCCAAAAGAGTGGCTGCAAGTGGACT |
| | PQSWVHQIALRMEVL | TCCAGAAGACAATGAAAGTCACAGGAGTAACTACTCAGGGAG |
| | GCEAQDLYGAGSPG | TAAAATCTCTGCTTACCAGCATGTATGTGAAGGAGTTCCTCAT |
| | AETAEQKLISEEDLSP | CTCCAGCAGTCAAGATGGCCATCAGTGGACTCTCTTTTTTCAG |
| | ATG | AATGGCAAAGTAAAGGTTTTTCAGGGAAATCAAGACTCCTTCA |
| | | CACCTGTGGTGAACTCTCTAGACCCACCGTTACTGACTCGCTA |
| | | CCTTCGAATTCACCCCCAGAGTTGGGTGCACCAGATTGCCCTG |
| | | AGGATGGAGGTTCTGGGCTGCGAGGCACAGGACCTCTACGGC |
| | | GCCGGATCACCTGGGGCCGAAACGGCCGAACAAAAACTCATC |
| | | TCAGAAGAGGATCTGTCACCTGCAACCGGTTGA |
| pBC0139 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTGTATTTGATGAAGGGAAAAGTTGGACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQGAPGSPAGSPTST | TGGAGAACTAAATGAACATTTGGGACTCCTGGGGCCATATATA |
| | EEGTSESATPESGPGS | AGAGCAGAAGTTGAAGATAATATCATGGTAACTTTCAGAAAT |
| | EPATSGSETPASSRQG | CAGGCCTCTCGTCCCTATTCCTTCTATTCTAGCCTTATTTCTTAT |
| | AEPRKNFVKPNETKT | GAGGAAGATCAGGGCGCGCCAGGTTCTCCTGCTGGCTCCCCCA |
| | YFWKVQHHMAPTKD | CCTCAACAGAAGAGGGGACAAGCGAAAGCGCTACGCCTGAGA |
| | EFDCKAWAYFSDVD | GTGGCCCTGGCTCTGAGCCAGCCACCTCCGGCTCTGAAACCCC |
| | LEKDVHSGLIGPLLV | TGCCTCGAGCAGGCAAGGAGCAGAACCTAGAAAAAACTTTGT |
| | CHTNTLNPAHGRQVT | CAAGCCTAATGAAACCAAAACTTACTTTTGGAAAGTGCAACAT |
| | VQEFALFFTIFDETKS | CATATGGCACCCACTAAAGATGAGTTTGACTGCAAAGCCTGGG |
| | WYFTENMERNCRAP | CTTATTTCTCTGATGTTGACCTGGAAAAAGATGTGCACTCAGG |
| | CNIQMEDPTFKENYR | CCTGATTGGACCCCTTCTGGTCTGCCACACTAACACACTGAAC |
| | FHAINGYIMDTLPGL | CCTGCTCATGGGAGACAAGTGACAGTACAGGAATTTGCTCTGT |
| | VMAQDQRIRWYLLS | TTTTCACCATCTTTGATGAGACCAAAAGCTGGTACTTCACTGA |
| | MGSNENIHSIHFSGHV | AAATATGGAAAGAAACTGCAGGGCTCCCTGCAATATCCAGAT |
| | FTVRKKEEYKMALY | GGAAGATCCCACTTTTAAAGAGAATTATCGCTTCCATGCAATC |
| | NLYPGVFETVEMLPS | AATGGCTACATAATGGATACACTACCTGGCTTAGTAATGGCTC |
| | KAGIWRVECLIGEHL | AGGATCAAAGGATTCGATGGTATCTGCTCAGCATGGGCAGCA |
| | HAGMSTLFLVYSNKC | ATGAAAACATCCATTCTATTCATTTCAGTGGACATGTGTTCACT |
| | QTPLGMASGHIRDFQI | GTACGAAAAAAAGAGGAGTATAAAATGGCACTGTACAATCTC |
| | TASGQYGQWAPKLA | TATCCAGGTGTTTTTGAGACAGTGGAAATGTTACCATCCAAAG |
| | RLHYSGSINAWSTKE | CTGGAATTTGGCGGGTGGAATGCCTTATTGGCGAGCATCTACA |
| | PFSWIKVDLLAPMIIH | TGCTGGGATGAGCACACTTTTTCTGGTGTACAGCAATAAGTGT |
| | GIKTQGARQKFSSLYI | CAGACTCCCCTGGGAATGGCTTCTGGACACATTAGAGATTTTC |
| | SQFIIMYSLDGKKWQ | AGATTACAGCTTCAGGACAATATGGACAGTGGGCCCCAAAGC |
| | TYRGNSTGTLMVFFG | TGGCCAGACTTCATTATTCCGGATCAATCAATGCCTGGAGCAC |
| | NVDSSGIKHNIFNPPII | CAAGGAGCCCTTTTCTTGGATCAAGGTGGATCTGTTGGCACCA |
| | ARYIRLHPTHYSIRST | ATGATTATTCACGGCATCAAGACCCAGGGTGCCCGTCAGAAGT |
| | LRMELMGCDLNSCS | TCTCCAGCCTCTACATCTCTCAGTTTATCATCATGTATAGTCTT |
| | MPLGMESKAISDAQI | GATGGGAAGAAGTGGCAGACTTATCGAGGAAATTCCACTGGA |
| | TASSYFTNMFATWSP | ACCTTAATGGTCTTCTTTGGCAATGTGGATTCATCTGGGATAA |
| | SKARLHLQGRSNAW | AACACAATATTTTTAACCCTCCAATTATTGCTCGATACATCCGT |
| | RPQVNNPKEWLQVD | TTGCACCCAACTCATTATAGCATTCGCAGCACTCTTCGCATGG |
| | FQKTMKVTGVTTQG | AGTTGATGGGCTGTGATTTAAATAGTTGCAGCATGCCATTGGG |
| | VKSLLTSMYVKEFLIS | AATGGAGAGTAAAGCAATATCAGATGCACAGATTACTGCTTC |
| | SSQDGHQWTLFFQNG | ATCCTACTTTACCAATATGTTTGCCACCTGGTCTCCTTCAAAAG |
| | KVKVFQGNQDSFTPV | CTCGACTTCACCTCCAAGGGAGGAGTAATGCCTGGAGACCTCA |
| | VNSLDPPLLTRYLRIH | GGTGAATAATCCAAAAGAGTGGCTGCAAGTGGACTTCCAGAA |
| | PQSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0140 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPGAPGSPA | TGGAGAACTAAATGAACATTTGGGACTCCTGGGGCCATATATA |
| | GSPTSTEEGTSESATP | AGAGCAGAAGTTGAAGATAATATCATGGTAACTTTCAGAAAT |
| | ESGPGSEPATSGSETP | CAGGCCTCTCGTCCCTATTCCTTCTATTCTAGCCTTATTTCTTAT |
| | ASSRKNFVKPNETKT | GAGGAAGATCAGAGGCAAGGAGCAGAACCTGGCGCGCCAGGT |
| | YFWKVQHHMAPTKD | TCTCCTGCTGGCTCCCCCACCTCAACAGAAGAGGGGACAAGCG |
| | EFDCKAWAYFSDVD | AAAGCGCTACGCCTGAGAGTGGCCCTGGCTCTGAGCCAGCCA |
| | LEKDVHSGLIGPLLV | CCTCCGGCTCTGAAACCCCTGCCTCGAGCAGAAAAAACTTTGT |
| | CHTNTLNPAHGRQVT | CAAGCCTAATGAAACCAAAACTTACTTTTGGAAAGTGCAACAT |
| | VQEFALFFTIFDETKS | CATATGGCACCCACTAAAGATGAGTTTGACTGCAAAGCCTGGG |
| | WYFTENMERNCRAP | CTTATTTCTCTGATGTTGACCTGGAAAAAGATGTGCACTCAGG |
| | CNIQMEDPTFKENYR | CCTGATTGGACCCCTTCTGGTCTGCCACACTAACACACTGAAC |
| | FHAINGYIMDTLPGL | CCTGCTCATGGGAGACAAGTGACAGTACAGGAATTTGCTCTGT |
| | VMAQDQRIRWYLLS | TTTTCACCATCTTTGATGAGACCAAAAGCTGGTACTTCACTGA |
| | MGSNENIHSIHFSGHV | AAATATGGAAAGAAACTGCAGGGCTCCCTGCAATATCCAGAT |
| | FTVRKKEEYKMALY | GGAAGATCCCACTTTTAAAGAGAATTATCGCTTCCATGCAATC |
| | NLYPGVFETVEMLPS | AATGGCTACATAATGGATACACTACCTGGCTTAGTAATGGCTC |
| | KAGIWRVECLIGEHL | AGGATCAAAGGATTCGATGGTATCTGCTCAGCATGGGCAGCA |
| | HAGMSTLFLVYSNKC | ATGAAAACATCCATTCTATTCATTTCAGTGGACATGTGTTCACT |
| | QTPLGMASGHIRDFQI | GTACGAAAAAAAGAGGAGTATAAAATGGCACTGTACAATCTC |
| | TASGQYGQWAPKLA | TATCCAGGTGTTTTTGAGACAGTGGAAATGTTACCATCCAAAG |
| | RLHYSGSINAWSTKE | CTGGAATTTGGCGGGTGGAATGCCTTATTGGCGAGCATCTACA |
| | PFSWIKVDLLAPMIIH | TGCTGGGATGAGCACACTTTTTCTGGTGTACAGCAATAAGTGT |
| | GIKTQGARQKFSSLYI | CAGACTCCCCTGGGAATGGCTTCTGGACACATTAGAGATTTTC |
| | SQFIIMYSLDGKKWQ | AGATTACAGCTTCAGGACAATATGGACAGTGGGCCCCAAAGC |
| | TYRGNSTGTLMVFFG | TGGCCAGACTTCATTATTCCGGATCAATCAATGCCTGGAGCAC |
| | NVDSSGIKHNIFNPPII | CAAGGAGCCCTTTTCTTGGATCAAGGTGGATCTGTTGGCACCA |
| | ARYIRLHPTHYSIRST | ATGATTATTCACGGCATCAAGACCCAGGGTGCCCGTCAGAAGT |
| | LRMELMGCDLNSCS | TCTCCAGCCTCTACATCTCTCAGTTTATCATCATGTATAGTCTT |
| | MPLGMESKAISDAQI | GATGGGAAGAAGTGGCAGACTTATCGAGGAAATTCCACTGGA |
| | TASSYFTNMFATWSP | ACCTTAATGGTCTTCTTTGGCAATGTGGATTCATCTGGGATAA |
| | SKARLHLQGRSNAW | AACACAATATTTTTAACCCTCCAATTATTGCTCGATACATCCGT |
| | RPQVNNPKEWLQVD | TTGCACCCAACTCATTATAGCATTCGCAGCACTCTTCGCATGG |
| | FQKTMKVTGVTTQG | AGTTGATGGGCTGTGATTTAAATAGTTGCAGCATGCCATTGGG |
| | VKSLLTSMYVKEFLIS | AATGGAGAGTAAAGCAATATCAGATGCACAGATTACTGCTTC |
| | SSQDGHQWTLFFQNG | ATCCTACTTTACCAATATGTTTGCCACCTGGTCTCCTTCAAAAG |
| | KVKVFQGNQDSFTPV | CTCGACTTCACCTCCAAGGGAGGAGTAATGCCTGGAGACCTCA |
| | VNSLDPPLLTRYLRIH | GGTGAATAATCCAAAAGAGTGGCTGCAAGTGGACTTCCAGAA |
| | PQSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0173 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKGAPGSPAGSPTS | CAGGCCTCTCGTCCCTATTCCTTCTATTCTAGCCTTATTTCTTAT |
| | TEEGTSESATPESGPG | GAGGAAGATCAGAGGCAAGGAGCAGAACCTAGAAAAAACTTT |
| | SEPATSGSETPASSDE | GTCAAGCCTAATGAAACCAAAACTTACTTTTGGAAAGTGCAAC |
| | FDCKAWAYFSDVDL | ATCATATGGCACCCACTAAAGGCGCGCCAGGTTCTCCTGCTGG |
| | EKDVHSGLIGPLLVC | CTCCCCCACCTCAACAGAAGAGGGGACAAGCGAAAGCGCTAC |
| | HTNTLNPAHGRQVTV | GCCTGAGAGTGGCCCTGGCTCTGAGCCAGCCACCTCCGGCTCT |
| | QEFALFFTIFDETKSW | GAAACCCCTGCCTCGAGCGATGAGTTTGACTGCAAAGCCTGGG |
| | YFTENMERNCRAPCN | CTTATTTCTCTGATGTTGACCTGGAAAAAGATGTGCACTCAGG |
| | IQMEDPTFKENYRFH | CCTGATTGGACCCCTTCTGGTCTGCCACACTAACACACTGAAC |
| | AINGYIMDTLPGLVM | CCTGCTCATGGGAGACAAGTGACAGTACAGGAATTTGCTCTGT |
| | AQDQPIRWYLLSMGS | TTTTCACCATCTTTGATGAGACCAAAAGCTGGTACTTCACTGA |
| | NENIHSIHFSGHVFTV | AAATATGGAAAGAAACTGCAGGGCTCCCTGCAATATCCAGAT |
| | RKKEEYKMALYNLY | GGAAGATCCCACTTTTAAAGAGAATTATCGCTTCCATGCAATC |
| | PGVFETVEMLPSKAG | AATGGCTACATAATGGATACACTACCTGGCTTAGTAATGGCTC |
| | IWRVECLIGEHLHAG | AGGATCAAAGGATTCGATGGTATCTGCTCAGCATGGGCAGCA |
| | MSTLFLVYSNKCQTP | ATGAAAACATCCATTCTATTCATTTCAGTGGACATGTGTTCACT |
| | LGMASGHIRDFQITAS | GTACGAAAAAAAGAGGAGTATAAAATGGCACTGTACAATCTC |
| | GQYGQWAPKLARLH | TATCCAGGTGTTTTTGAGACAGTGGAAATGTTACCATCCAAAG |
| | YSGSINAWSTKEPFS | CTGGAATTTGGCGGGTGGAATGCCTTATTGGCGAGCATCTACA |
| | WIKVDLLAPMIIHGIK | TGCTGGGATGAGCACACTTTTTCTGGTGTACAGCAATAAGTGT |
| | TQGARQKFSSLYISQF | CAGACTCCCCTGGGAATGGCTTCTGGACACATTAGAGATTTTC |
| | IIMYSLDGKKWQTYR | AGATTACAGCTTCAGGACAATATGGACAGTGGGCCCCAAAGC |
| | GNSTGTLMVFFGNVD | TGGCCAGACTTCATTATTCCGGATCAATCAATGCCTGGAGCAC |
| | SSGIKHNIFNPPIIARYI | CAAGGAGCCCTTTTCTTGGATCAAGGTGGATCTGTTGGCACCA |
| | RLHPTHYSIRSTLRME | ATGATTATTCACGGCATCAAGACCCAGGGTGCCCGTCAGAAGT |
| | LMGCDLNSCSMPLG | TCTCCAGCCTCTACATCTCTCAGTTTATCATCATGTATAGTCTT |
| | MESKAISDAQITASSY | GATGGGAAGAAGTGGCAGACTTATCGAGGAAATTCCACTGGA |
| | FTNMFATWSPSKARL | ACCTTAATGGTCTTCTTTGGCAATGTGGATTCATCTGGGATAA |
| | HLQGRSNAWRPQVN | AACACAATATTTTTAACCCTCCAATTATTGCTCGATACATCCGT |
| | NPKEWLQVDFQKTM | TTGCACCCAACTCATTATAGCATTCGCAGCACTCTTCGCATGG |
| | KVTGVTTQGVKSLLT | AGTTGATGGGCTGTGATTTAAATAGTTGCAGCATGCCATTGGG |
| | SMYVKEFLISSSQDG | AATGGAGAGTAAAGCAATATCAGATGCACAGATTACTGCTTC |
| | HQWTLFFQNGKVKV | ATCCTACTTTACCAATATGTTTGCCACCTGGTCTCCTTCAAAAG |
| | FQGNQDSFTPVVNSL | CTCGACTTCACCTCCAAGGGAGGAGTAATGCCTGGAGACCTCA |
| | DPPLLTRYLRIHPQSW | GGTGAATAATCCAAAAGAGTGGCTGCAAGTGGACTTCCAGAA |
| | VHQIALRMEVLGCEA | GACAATGAAAGTCACAGGAGTAACTACTCAGGGAGTAAAATC |
| | QDLYGAGSPGAETAE | TCTGCTTACCAGCATGTATGTGAAGGAGTTCCTCATCTCCAGC |
| | QKLISEEDLSPATG | AGTCAAGATGGCCATCAGTGGACTCTCTTTTTTCAGAATGGCA |
| | | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0174 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNGAPGSP | GTCAAGCCTAATGAAACCAAAACTTACTTTTGGAAAGTGCAAC |
| | AGSPTSTEEGTSESAT | ATCATATGGCACCCACTAAAGATGAGTTTGACTGCAAAGCCTG |
| | PESGPGSEPATSGSET | GGCTTATTTCTCTGATGTTGACCTGGAAAAAGATGTGCACTCA |
| | PASSTLNPAHGRQVT | GGCCTGATTGGACCCCTTCTGGTCTGCCACACTAACGGCGCGC |
| | VQEFALFFTIFDETKS | CAGGTTCTCCTGCTGGCTCCCCCACCTCAACAGAAGAGGGGAC |
| | WYFTENMERNCRAP | AAGCGAAAGCGCTACGCCTGAGAGTGGCCCTGGCTCTGAGCC |
| | CNIQMEDPTFKENYR | AGCCACCTCCGGCTCTGAAACCCCTGCCTCGAGCACACTGAAC |
| | FHAINGYIMDTLPGL | CCTGCTCATGGGAGACAAGTGACAGTACAGGAATTTGCTCTGT |
| | VMAQDQRIRWYLLS | TTTTCACCATCTTTGATGAGACCAAAAGCTGGTACTTCACTGA |
| | MGSNENIHSIHFSGHV | AAATATGGAAAGAAACTGCAGGGCTCCCTGCAATATCCAGAT |
| | FTVRKKEEYKMALY | GGAAGATCCCACTTTTAAAGAGAATTATCGCTTCCATGCAATC |
| | NLYPGVFETVEMLPS | AATGGCTACATAATGGATACACTACCTGGCTTAGTAATGGCTC |
| | KAGIWRVECLIGEHL | AGGATCAAAGGATTCGATGGTATCTGCTCAGCATGGGCAGCA |
| | HAGMSTLFLVYSNKC | ATGAAAACATCCATTCTATTCATTTCAGTGGACATGTGTTCACT |
| | QTPLGMASGHIRDFQI | GTACGAAAAAAAGAGGAGTATAAAATGGCACTGTACAATCTC |
| | TASGQYGQWAPKLA | TATCCAGGTGTTTTTGAGACAGTGGAAATGTTACCATCCAAAG |
| | RLHYSGSINAWSTKE | CTGGAATTTGGCGGGTGGAATGCCTTATTGGCGAGCATCTACA |
| | PFSWIKVDLLAPMIIH | TGCTGGGATGAGCACACTTTTTCTGGTGTACAGCAATAAGTGT |
| | GIKTQGARQKFSSLYI | CAGACTCCCCTGGGAATGGCTTCTGGACACATTAGAGATTTTC |
| | SQFIIMYSLDGKKWQ | AGATTACAGCTTCAGGACAATATGGACAGTGGGCCCCAAAGC |
| | TYRGNSTGTLMVFFG | TGGCCAGACTTCATTATTCCGGATCAATCAATGCCTGGAGCAC |
| | NVDSSGIKHNIFNPPII | CAAGGAGCCCTTTTCTTGGATCAAGGTGGATCTGTTGGCACCA |
| | ARYIRLHPTHYSIRST | ATGATTATTCACGGCATCAAGACCCAGGGTGCCCGTCAGAAGT |
| | LRMELMGCDLNSCS | TCTCCAGCCTCTACATCTCTCAGTTTATCATCATGTATAGTCTT |
| | MPLGMESKAISDAQI | GATGGGAAGAAGTGGCAGACTTATCGAGGAAATTCCACTGGA |
| | TASSYFTNMFATWSP | ACCTTAATGGTCTTCTTTGGCAATGTGGATTCATCTGGGATAA |
| | SKARLHLQGRSNAW | AACACAATATTTTTAACCCTCCAATTATTGCTCGATACATCCGT |
| | RPQVNNPKEWLQVD | TTGCACCCAACTCATTATAGCATTCGCAGCACTCTTCGCATGG |
| | FQKTMKVTGVTTQG | AGTTGATGGGCTGTGATTTAAATAGTTGCAGCATGCCATTGGG |
| | VKSLLTSMYVKEFLIS | AATGGAGAGTAAAGCAATATCAGATGCACAGATTACTGCTTC |
| | SSQDGHQWTLFFQNG | ATCCTACTTTACCAATATGTTTGCCACCTGGTCTCCTTCAAAAG |
| | KVKVFQGNQDSFTPV | CTCGACTTCACCTCCAAGGGAGGAGTAATGCCTGGAGACCTCA |
| | VNSLDPPLLTRYLRIH | GGTGAATAATCCAAAAGAGTGGCTGCAAGTGGACTTCCAGAA |
| | PQSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0175 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMEG | GGCTTATTTCTCTGATGTTGACCTGGAAAAAGATGTGCACTCA |
| | APGSPAGSPTSTEEGT | GGCCTGATTGGACCCCTTCTGGTCTGCCACACTAACACACTGA |
| | SESATPESGPGSEPAT | ACCCTGCTCATGGGAGACAAGTGACAGTACAGGAATTTGCTCT |
| | SGSETPASSRNCRAPC | GTTTTTCACCATCTTTGATGAGACCAAAAGCTGGTACTTCACT |
| | NIQMEDPTFKENYRF | GAAAATATGGAAGGCGCGCCAGGTTCTCCTGCTGGCTCCCCCA |
| | HAINGYIMDTLPGLV | CCTCAACAGAAGAGGGGACAAGCGAAAGCGCTACGCCTGAGA |
| | MAQDQRIRWYLLSM | GTGGCCCTGGCTCTGAGCCAGCCACCTCCGGCTCTGAAACCCC |
| | GSNENIHSIHFSGHVF | TGCCTCGAGCAGAAACTGCAGGGCTCCCTGCAATATCCAGATG |
| | TVRKKEEYKMALYN | GAAGATCCCACTTTTAAAGAGAATTATCGCTTCCATGCAATCA |
| | LYPGVFETVEMLPSK | ATGGCTACATAATGGATACACTACCTGGCTTAGTAATGGCTCA |
| | AGIWRVECLIGEHLH | GGATCAAAGGATTCGATGGTATCTGCTCAGCATGGGCAGCAAT |
| | AGMSTLFLVYSNKCQ | GAAAACATCCATTCTATTCATTTCAGTGGACATGTGTTCACTGT |
| | TPLGMASGHIRDFQIT | ACGAAAAAAAGAGGAGTATAAAATGGCACTGTACAATCTCTA |
| | ASGQYGQWAPKLAR | TCCAGGTGTTTTTGAGACAGTGGAAATGTTACCATCCAAAGCT |
| | LHYSGSINAWSTKEP | GGAATTTGGCGGGTGGAATGCCTTATTGGCGAGCATCTACATG |
| | FSWIKVDLLAPMIIHG | CTGGGATGAGCACACTTTTTCTGGTGTACAGCAATAAGTGTCA |
| | IKTQGARQKFSSLYIS | GACTCCCCTGGGAATGGCTTCTGGACACATTAGAGATTTTCAG |
| | QFIIMYSLDGKKWQT | ATTACAGCTTCAGGACAATATGGACAGTGGGCCCCAAAGCTG |
| | YRGNSTGTLMVFFGN | GCCAGACTTCATTATTCCGGATCAATCAATGCCTGGAGCACCA |
| | VDSSGIKHNIFNPPIIA | AGGAGCCCTTTTCTTGGATCAAGGTGGATCTGTTGGCACCAAT |
| | RYIRLHPTHYSIRSTL | GATTATTCACGGCATCAAGACCCAGGGTGCCCGTCAGAAGTTC |
| | RMELMGCDLNSCSM | TCCAGCCTCTACATCTCTCAGTTTATCATCATGTATAGTCTTGA |
| | PLGMESKAISDAQITA | TGGGAAGAAGTGGCAGACTTATCGAGGAAATTCCACTGGAAC |
| | SSYFTNMFATWSPSK | CTTAATGGTCTTCTTTGGCAATGTGGATTCATCTGGGATAAAA |
| | ARLHLQGRSNAWRP | CACAATATTTTTAACCCTCCAATTATTGCTCGATACATCCGTTT |
| | QVNNPKEWLQVDFQ | GCACCCAACTCATTATAGCATTCGCAGCACTCTTCGCATGGAG |
| | KTMKVTGVTTQGVK | TTGATGGGCTGTGATTTAAATAGTTGCAGCATGCCATTGGGAA |
| | SLLTSMYVKEFLISSS | TGGAGAGTAAAGCAATATCAGATGCACAGATTACTGCTTCATC |
| | QDGHQWTLFFQNGK | CTACTTTACCAATATGTTTGCCACCTGGTCTCCTTCAAAAGCTC |
| | VKVFQGNQDSFTPVV | GACTTCACCTCCAAGGGAGGAGTAATGCCTGGAGACCTCAGG |
| | NSLDPPLLTRYLRIHP | TGAATAATCCAAAAGAGTGGCTGCAAGTGGACTTCCAGAAGA |
| | QSWVHQIALRMEVL | CAATGAAAGTCACAGGAGTAACTACTCAGGGAGTAAAATCTC |
| | GCEAQDLYGAGSPG | TGCTTACCAGCATGTATGTGAAGGAGTTCCTCATCTCCAGCAG |
| | AETAEQKLISEEDLSP | TCAAGATGGCCATCAGTGGACTCTCTTTTTTCAGAATGGCAAA |
| | ATG | GTAAAGGTTTTTCAGGGAAATCAAGACTCCTTCACACCTGTGG |
| | | TGAACTCTCTAGACCCACCGTTACTGACTCGCTACCTTCGAATT |
| | | CACCCCCAGAGTTGGGTGCACCAGATTGCCCTGAGGATGGAG |
| | | GTTCTGGGCTGCGAGGCACAGGACCTCTACGGCGCCGGATCAC |
| | | CTGGGGCCGAAACGGCCGAACAAAAACTCATCTCAGAAGAGG |
| | | ATCTGTCACCTGCAACCGGTTGA |
| pBC0176 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRGAPGSPAGSPTS | GGCCTGATTGGACCCCTTCTGGTCTGCCACACTAACACACTGA |
| | TEEGTSESATPESGPG | ACCCTGCTCATGGGAGACAAGTGACAGTACAGGAATTTGCTCT |
| | SEPATSGSETPASSAP | GTTTTTCACCATCTTTGATGAGACCAAAAGCTGGTACTTCACT |
| | CNIQMEDPTFKENYR | GAAAATATGGAAAGAAACTGCAGGGGCGCGCCAGGTTCTCCT |
| | FHAINGYIMDTLPGL | GCTGGCTCCCCCACCTCAACAGAAGAGGGGACAAGCGAAAGC |
| | VMAQDQRIRWYLLS | GCTACGCCTGAGAGTGGCCCTGGCTCTGAGCCAGCCACCTCCG |
| | MGSNENIHSIHFSGHV | GCTCTGAAACCCCTGCCTCGAGCGCTCCCTGCAATATCCAGAT |
| | FTVRKKEEYKMALY | GGAAGATCCCACTTTTAAAGAGAATTATCGCTTCCATGCAATC |
| | NLYPGVFETVEMLPS | AATGGCTACATAATGGATACACTACCTGGCTTAGTAATGGCTC |
| | KAGIWRVECLIGEHL | AGGATCAAAGGATTCGATGGTATCTGCTCAGCATGGGCAGCA |
| | HAGMSTLFLVYSNKC | ATGAAAACATCCATTCTATTCATTTCAGTGGACATGTGTTCACT |
| | QTPLGMASGHIRDFQI | GTACGAAAAAAAGAGGAGTATAAAATGGCACTGTACAATCTC |
| | TASGQYGQWAPKLA | TATCCAGGTGTTTTTGAGACAGTGGAAATGTTACCATCCAAAG |
| | RLHYSGSINAWSTKE | CTGGAATTTGGCGGGTGGAATGCCTTATTGGCGAGCATCTACA |
| | PFSWIKVDLLAPMIIH | TGCTGGGATGAGCACACTTTTTCTGGTGTACAGCAATAAGTGT |
| | GIKTQGARQKFSSLYI | CAGACTCCCCTGGGAATGGCTTCTGGACACATTAGAGATTTTC |
| | SQFIIMYSLDGKKWQ | AGATTACAGCTTCAGGACAATATGGACAGTGGGCCCCAAAGC |
| | TYRGNSTGTLMVFFG | TGGCCAGACTTCATTATTCCGGATCAATCAATGCCTGGAGCAC |
| | NVDSSGIKHNIFNPPII | CAAGGAGCCCTTTTCTTGGATCAAGGTGGATCTGTTGGCACCA |
| | ARYIRLHPTHYSIRST | ATGATTATTCACGGCATCAAGACCCAGGGTGCCCGTCAGAAGT |
| | LRMELMGCDLNSCS | TCTCCAGCCTCTACATCTCTCAGTTTATCATCATGTATAGTCTT |
| | MPLGMESKAISDAQI | GATGGGAAGAAGTGGCAGACTTATCGAGGAAATTCCACTGGA |
| | TASSYFTNMFATWSP | ACCTTAATGGTCTTCTTTGGCAATGTGGATTCATCTGGGATAA |
| | SKARLHLQGRSNAW | AACACAATATTTTTAACCCTCCAATTATTGCTCGATACATCCGT |
| | RPQVNNPKEWLQVD | TTGCACCCAACTCATTATAGCATTCGCAGCACTCTTCGCATGG |
| | FQKTMKVTGVTTQG | AGTTGATGGGCTGTGATTTAAATAGTTGCAGCATGCCATTGGG |
| | VKSLLTSMYVKEFLIS | AATGGAGAGTAAAGCAATATCAGATGCACAGATTACTGCTTC |
| | SSQDGHQWTLFFQNG | ATCCTACTTTACCAATATGTTTGCCACCTGGTCTCCTTCAAAAG |
| | KVKVFQGNQDSFTPV | CTCGACTTCACCTCCAAGGGAGGAGTAATGCCTGGAGACCTCA |
| | VNSLDPPLLTRYLRIH | GGTGAATAATCCAAAAGAGTGGCTGCAAGTGGACTTCCAGAA |
| | PQSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0177 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNGAPGSPAG | GGCCTGATTGGACCCCTTCTGGTCTGCCACACTAACACACTGA |
| | SPTSTEEGTSESATPE | ACCCTGCTCATGGGAGACAAGTGACAGTACAGGAATTTGCTCT |
| | SGPGSEPATSGSETPA | GTTTTTCACCATCTTTGATGAGACCAAAAGCTGGTACTTCACT |
| | SSIQMEDPTFKENYRF | GAAAATATGGAAAGAAACTGCAGGGCTCCCTGCAATGGCGCG |
| | HAINGYIMDTLPGLV | CCAGGTTCTCCTGCTGGCTCCCCCACCTCAACAGAAGAGGGGA |
| | MAQDQRIRWYLLSM | CAAGCGAAAGCGCTACGCCTGAGAGTGGCCCTGGCTCTGAGC |
| | GSNENIHSIHFSGHVF | CAGCCACCTCCGGCTCTGAAACCCCTGCCTCGAGCATCCAGAT |
| | TVRKKEEYKMALYN | GGAAGATCCCACTTTTAAAGAGAATTATCGCTTCCATGCAATC |
| | LYPGVFETVEMLPSK | AATGGCTACATAATGGATACACTACCTGGCTTAGTAATGGCTC |
| | AGIWRVECLIGEHLH | AGGATCAAAGGATTCGATGGTATCTGCTCAGCATGGGCAGCA |
| | AGMSTLFLVYSNKCQ | ATGAAAACATCCATTCTATTCATTTCAGTGGACATGTGTTCACT |
| | TPLGMASGHIRDFQIT | GTACGAAAAAAAGAGGAGTATAAAATGGCACTGTACAATCTC |
| | ASGQYGQWAPKLAR | TATCCAGGTGTTTTTGAGACAGTGGAAATGTTACCATCCAAAG |
| | LHYSGSINAWSTKEP | CTGGAATTTGGCGGGTGGAATGCCTTATTGGCGAGCATCTACA |
| | FSWIKVDLLAPMIIHG | TGCTGGGATGAGCACACTTTTTCTGGTGTACAGCAATAAGTGT |
| | IKTQGARQKFSSLYIS | CAGACTCCCCTGGGAATGGCTTCTGGACACATTAGAGATTTTC |
| | QFIIMYSLDGKKWQT | AGATTACAGCTTCAGGACAATATGGACAGTGGGCCCCAAAGC |
| | YRGNSTGTLMVFFGN | TGGCCAGACTTCATTATTCCGGATCAATCAATGCCTGGAGCAC |
| | VDSSGIKHNIFNPPIIA | CAAGGAGCCCTTTTCTTGGATCAAGGTGGATCTGTTGGCACCA |
| | RYIRLHPTHYSIRSTL | ATGATTATTCACGGCATCAAGACCCAGGGTGCCCGTCAGAAGT |
| | RMELMGCDLNSCSM | TCTCCAGCCTCTACATCTCTCAGTTTATCATCATGTATAGTCTT |
| | PLGMESKAISDAQITA | GATGGGAAGAAGTGGCAGACTTATCGAGGAAATTCCACTGGA |
| | SSYFTNMFATWSPSK | ACCTTAATGGTCTTCTTTGGCAATGTGGATTCATCTGGGATAA |
| | ARLHLQGRSNAWRP | AACACAATATTTTTAACCCTCCAATTATTGCTCGATACATCCGT |
| | QVNNPKEWLQVDFQ | TTGCACCCAACTCATTATAGCATTCGCAGCACTCTTCGCATGG |
| | KTMKVTGVTTQGVK | AGTTGATGGGCTGTGATTTAAATAGTTGCAGCATGCCATTGGG |
| | SLLTSMYVKEFLISSS | AATGGAGAGTAAAGCAATATCAGATGCACAGATTACTGCTTC |
| | QDGHQWTLFFQNGK | ATCCTACTTTACCAATATGTTTGCCACCTGGTCTCCTTCAAAAG |
| | VKVFQGNQDSFTPVV | CTCGACTTCACCTCCAAGGGAGGAGTAATGCCTGGAGACCTCA |
| | NSLDPPLLTRYLRIHP | GGTGAATAATCCAAAAGAGTGGCTGCAAGTGGACTTCCAGAA |
| | QSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0178 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDGAPGS | GTTTTTCACCATCTTTGATGAGACCAAAAGCTGGTACTTCACT |
| | PAGSPTSTEEGTSESA | GAAAATATGGAAAGAAACTGCAGGGCTCCCTGCAATATCCAG |
| | TPESGPGSEPATSGSE | ATGGAAGATCCCACTTTTAAAGAGAATTATCGCTTCCATGCAA |
| | TPASSQRIRWYLLSM | TCAATGGCTACATAATGGATACACTACCTGGCTTAGTAATGGC |
| | GSNENIHSIHFSGHVF | TCAGGATGGCGCGCCAGGTTCTCCTGCTGGCTCCCCCACCTCA |
| | TVRKKEEYKMALYN | ACAGAAGAGGGGACAAGCGAAAGCGCTACGCCTGAGAGTGGC |
| | LYPGVFETVEMLPSK | CCTGGCTCTGAGCCAGCCACCTCCGGCTCTGAAACCCCTGCCT |
| | AGIWRVECLIGEHLH | CGAGCCAAAGGATTCGATGGTATCTGCTCAGCATGGGCAGCA |
| | AGMSTLFLVYSNKCQ | ATGAAAACATCCATTCTATTCATTTCAGTGGACATGTGTTCACT |
| | TPLGMASGHIRDFQIT | GTACGAAAAAAAGAGGAGTATAAAATGGCACTGTACAATCTC |
| | ASGQYGQWAPKLAR | TATCCAGGTGTTTTTGAGACAGTGGAAATGTTACCATCCAAAG |
| | LHYSGSINAWSTKEP | CTGGAATTTGGCGGGTGGAATGCCTTATTGGCGAGCATCTACA |
| | FSWIKVDLLAPMIIHG | TGCTGGGATGAGCACACTTTTTCTGGTGTACAGCAATAAGTGT |
| | IKTQGARQKFSSLYIS | CAGACTCCCCTGGGAATGGCTTCTGGACACATTAGAGATTTTC |
| | QFIIMYSLDGKKWQT | AGATTACAGCTTCAGGACAATATGGACAGTGGGCCCCAAAGC |
| | YRGNSTGTLMVFFGN | TGGCCAGACTTCATTATTCCGGATCAATCAATGCCTGGAGCAC |
| | VDSSGIKHNIFNPPIIA | CAAGGAGCCCTTTTCTTGGATCAAGGTGGATCTGTTGGCACCA |
| | RYIRLHPTHYSIRSTL | ATGATTATTCACGGCATCAAGACCCAGGGTGCCCGTCAGAAGT |
| | RMELMGCDLNSCSM | TCTCCAGCCTCTACATCTCTCAGTTTATCATCATGTATAGTCTT |
| | PLGMESKAISDAQITA | GATGGGAAGAAGTGGCAGACTTATCGAGGAAATTCCACTGGA |
| | SSYFTNMFATWSPSK | ACCTTAATGGTCTTCTTTGGCAATGTGGATTCATCTGGGATAA |
| | ARLHLQGRSNAWRP | AACACAATATTTTTAACCCTCCAATTATTGCTCGATACATCCGT |
| | QVNNPKEWLQVDFQ | TTGCACCCAACTCATTATAGCATTCGCAGCACTCTTCGCATGG |
| | KTMKVTGVTTQGVK | AGTTGATGGGCTGTGATTTAAATAGTTGCAGCATGCCATTGGG |
| | SLLTSMYVKEFLISSS | AATGGAGAGTAAAGCAATATCAGATGCACAGATTACTGCTTC |
| | QDGHQWTLFFQNGK | ATCCTACTTTACCAATATGTTTGCCACCTGGTCTCCTTCAAAAG |
| | VKVFQGNQDSFTPVV | CTCGACTTCACCTCCAAGGGAGGAGTAATGCCTGGAGACCTCA |
| | NSLDPPLLTRYLRIHP | GGTGAATAATCCAAAAGAGTGGCTGCAAGTGGACTTCCAGAA |
| | QSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0141 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNGAPGSPAGSPT | CTGTACGAAAAAAAGAGGAGTATAAAATGGCACTGTACAATC |
| | STEEGTSESATPESGP | TCTATCCAGGTGTTTTTGAGACAGTGGAAATGTTACCATCCAA |
| | GSEPATSGSETPASSK | AGCTGGAATTTGGCGGGTGGAATGCCTTATTGGCGAGCATCTA |
| | CQTPLGMASGHIRDF | CATGCTGGGATGAGCACACTTTTTCTGGTGTACAGCAATGGCG |
| | QITASGQYGQWAPKL | CGCCAGGTTCTCCTGCTGGCTCCCCCACCTCAACAGAAGAGGG |
| | ARLHYSGSINAWSTK | GACAAGCGAAAGCGCTACGCCTGAGAGTGGCCCTGGCTCTGA |
| | EPFSWIKVDLLAPMII | GCCAGCCACCTCCGGCTCTGAAACCCCTGCCTCGAGCAAGTGT |
| | HGIKTQGARQKFSSL | CAGACTCCCCTGGGAATGGCTTCTGGACACATTAGAGATTTTC |
| | YISQFIIMYSLDGKKW | AGATTACAGCTTCAGGACAATATGGACAGTGGGCCCCAAAGC |
| | QTYRGNSTGTLMVFF | TGGCCAGACTTCATTATTCCGGATCAATCAATGCCTGGAGCAC |
| | GNVDSSGIKHNIFNPP | CAAGGAGCCCTTTTCTTGGATCAAGGTGGATCTGTTGGCACCA |
| | IIARYIRLHPTHYSIRS | ATGATTATTCACGGCATCAAGACCCAGGGTGCCCGTCAGAAGT |
| | TLRMELMGCDLNSCS | TCTCCAGCCTCTACATCTCTCAGTTTATCATCATGTATAGTCTT |
| | MPLGMESKAISDAQI | GATGGGAAGAAGTGGCAGACTTATCGAGGAAATTCCACTGGA |
| | TASSYFTNMFATWSP | ACCTTAATGGTCTTCTTTGGCAATGTGGATTCATCTGGGATAA |
| | SKARLHLQGRSNAW | AACACAATATTTTTAACCCTCCAATTATTGCTCGATACATCCGT |
| | RPQVNNPKEWLQVD | TTGCACCCAACTCATTATAGCATTCGCAGCACTCTTCGCATGG |
| | FQKTMKVTGVTTQG | AGTTGATGGGCTGTGATTTAAATAGTTGCAGCATGCCATTGGG |
| | VKSLLTSMYVKEFLIS | AATGGAGAGTAAAGCAATATCAGATGCACAGATTACTGCTTC |
| | SSQDGHQWTLFFQNG | ATCCTACTTTACCAATATGTTTGCCACCTGGTCTCCTTCAAAAG |
| | KVKVFQGNQDSFTPV | CTCGACTTCACCTCCAAGGGAGGAGTAATGCCTGGAGACCTCA |
| | VNSLDPPLLTRYLRIH | GGTGAATAATCCAAAAGAGTGGCTGCAAGTGGACTTCCAGAA |
| | PQSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0179 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFGAPGSP | CATGCTGGGATGAGCACACTTTTTCTGGTGTACAGCAATAAGT |
| | AGSPTSTEEGTSESAT | GTCAGACTCCCCTGGGAATGGCTTCTGGACACATTAGAGATTT |
| | PESGPGSEPATSGSET | TCAGATTACAGCTTCAGGACAATATGGACAGTGGGCCCCAAA |
| | PASSSWIKVDLLAPMI | GCTGGCCAGACTTCATTATTCCGGATCAATCAATGCCTGGAGC |
| | IHGIKTQGARQKFSSL | ACCAAGGAGCCCTTTGGCGCGCCAGGTTCTCCTGCTGGCTCCC |
| | YISQFIIMYSLDGKKW | CCACCTCAACAGAAGAGGGGACAAGCGAAAGCGCTACGCCTG |
| | QTYRGNSTGTLMVFF | AGAGTGGCCCTGGCTCTGAGCCAGCCACCTCCGGCTCTGAAAC |
| | GNVDSSGIKHNIFNPP | CCCTGCCTCGAGCTCTTGGATCAAGGTGGATCTGTTGGCACCA |
| | IIARYIRLHPTHYSIRS | ATGATTATTCACGGCATCAAGACCCAGGGTGCCCGTCAGAAGT |
| | TLRMELMGCDLNSCS | TCTCCAGCCTCTACATCTCTCAGTTTATCATCATGTATAGTCTT |
| | MPLGMESKAISDAQI | GATGGGAAGAAGTGGCAGACTTATCGAGGAAATTCCACTGGA |
| | TASSYFTNMFATWSP | ACCTTAATGGTCTTCTTTGGCAATGTGGATTCATCTGGGATAA |
| | SKARLHLQGRSNAW | AACACAATATTTTTAACCCTCCAATTATTGCTCGATACATCCGT |
| | RPQVNNPKEWLQVD | TTGCACCCAACTCATTATAGCATTCGCAGCACTCTTCGCATGG |
| | FQKTMKVTGVTTQG | AGTTGATGGGCTGTGATTTAAATAGTTGCAGCATGCCATTGGG |
| | VKSLLTSMYVKEFLIS | AATGGAGAGTAAAGCAATATCAGATGCACAGATTACTGCTTC |
| | SSQDGHQWTLFFQNG | ATCCTACTTTACCAATATGTTTGCCACCTGGTCTCCTTCAAAAG |
| | KVKVFQGNQDSFTPV | CTCGACTTCACCTCCAAGGGAGGAGTAATGCCTGGAGACCTCA |
| | VNSLDPPLLTRYLRIH | GGTGAATAATCCAAAAGAGTGGCTGCAAGTGGACTTCCAGAA |
| | PQSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC205 | | ATGCAAATAGAGCTCTCCACCTGCTTCTTTCTGTGCCTTTTGCG |
| | | ATTCTGCTTTAGTGCCACCAGAAGATACTACCTGGGTGCAGTG |
| | | GAACTGTCATGGGACTATATGCAAAGTGATCTCGGTGAGCTGC |
| | | CTGTGGACGCAAGATTTCCTCCTAGAGTGCCAAAATCTTTTCC |
| | | ATTCAACACCTCAGTCGTGTACAAAAAGACTCTGTTTGTAGAA |
| | | TTCACGGTTCACCTTTTCAACATCGCTAAGCCAAGGCCACCCT |
| | | GGATGGGTCTGCTAGGTCCTACCATCCAGGCTGAGGTTTATGA |
| | | TACAGTGGTCATTACACTTAAGAACATGGCTTCCCATCCTGTC |
| | | AGCCTTCATGCTGTTGGTGTATCCTACTGGAAAGCGTCTGAGG |
| | | GAGCTGAATATGATGATCAGACCAGTCAAAGGGAGAAAGAAG |
| | | ATGATAAAGTTTTCCCTGGTGGAAGCCATACATATGTCTGGCA |
| | | GGTCCTGAAAGAGAATGGTCCAATGGCCTCTGACCCACTGTGC |
| | | CTTACCTACTCATATCTTTCTCATGTGGACCTGGTAAAAGACTT |
| | | GAATTCAGGCCTCATTGGAGCCCTACTAGTATGTAGAGAAGGG |
| | | AGTCTGGCCAAGGAAAAGACACAGACCTTGCACAAATTTATA |
| | | CTACTTTTTGCTGTATTTGATGAAGGGAAAAGTTGGCACTCAG |
| | | AAACAAAGAACTCCTTGATGCAGGATAGGGATGCTGCATCTG |
| | | CTCGGGCCTGGCCTAAAATGCACACAGTCAATGGTTATGTAAA |
| | | CAGTTCTCTGCCAGGTCTGATTGGATGCCACAGGAAATCAGTC |
| | | TATTGGCATGTGATTGGAATGGGCACCACTCCTGAAGTGCACT |
| | | CAATATTCCTCGAAGGTCACACATTTCTTGTGAGGAACCATCG |
| | | CCAGGCGTCCTTGGAAATCTCGCCAATAACTTTCCTTACTGCTC |
| | | AAACACTCTTGATGGACCTTGGACAGTTTCTACTGTTTTGTCAT |
| | | ATCTCTTCCCACCAACATGATGGCATGGAAGCCTATGTCAAAG |
| | | TAGACAGCTGTCCAGAGGAACCCCAACTACGAATGAAAAATA |
| | | ATGAAGAAGCGGAAGATTATGATGATGATCTTACTGATTCTGA |
| | | AATGGATGTGGTCAGGTTTGATGATGACAACTCTCCTTCCTTT |
| | | ATCCAAATTCGCTCAGTTGCCAAGAAGCATCCTAAAACTTGGG |
| | | TACATTACATTGCTGCTGAAGAGGAGGACTGGGACTATGCTCC |
| | | CTTAGTCCTCGCCCCCGATGACAGAAGTTATAAAAGTCAATAT |
| | | TTGAACAATGGCCCTCAGCGGATTGGTAGGAAGTACAAAAAA |
| | | GTCCGATTTATGGCATACACAGATGAAACCTTTAAGACTCGTG |
| | | AAGCTATTCAGCATGAATCAGGAATCTTGGGACCTTTACTTTA |
| | | TGGGGAAGTTGGAGACACACTGTTGATTATATTTAAGAATCAA |
| | | GCAAGCAGACCATATAACATCTACCCTCACGGAATCACTGATG |
| | | TCCGTCCTTTGTATTCAAGGAGATTACCAAAAGGTGTAAAACA |
| | | TTTGAAGGATTTTCCAATTCTGCCAGGAGAAATATTCAAATAT |
| | | AAATGGACAGTGACTGTAGAAGATGGGCCAACTAAATCAGAT |
| | | CCTCGGTGCCTGACCCGCTATTACTCTAGTTTCGTTAATATGGA |
| | | GAGAGATCTAGCTTCAGGACTCATTGGCCCTCTCCTCATCTGC |
| | | TACAAAGAATCTGTAGATCAAAGAGGAAACCAGATAATGTCA |
| | | GACAAGAGGAATGTCATCCTGTTTTCTGTATTTGATGAGAACC |
| | | GAAGCTGGTACCTCACAGAGAATATACAACGCTTTCTCCCCAA |
| | | TCCAGCTGGAGTGCAGCTTGAGGATCCAGAGTTCCAAGCCTCC |
| | | AACATCATGCACAGCATCAATGGCTATGTTTTTGATAGTTTGC |
| | | AGTTGTCAGTTTGTTTGCATGAGGTGGCATACTGGTACATTCT |
| | | AAGCATTGGAGCACAGACTGACTTCCTTTCTGTGTTCTTCTCTG |
| | | GATATACCTTCAAACACAAAATGGTCTATGAAGATACACTCAC |
| | | CCTATTCCCATTCTCAGGAGAAACTGTGTTCATGTCGATGGAA |
| | | AACCCAGGTCTATGGATTCTGGGGTGCCACAACTCAGACTTTC |
| | | GGAACAGAGGCATGACCGCCTTACTGAAGGTTTCTAGTTGTGA |
| | | CAAGAACACTGGTGATTATTACGAGGACAGTTATGAAGATATT |
| | | TCAGCATACTTGCTGAGTAAAAACAATGCCATTGAACCAAGA |
| | | AGCTTCTCCCAGAATGGGTCTCCCGCGCCAGGGTCTCCCCCAC |
| | | CAGTCTTGAAACGCCATCAACGGGAAATAACTCGTACTACTCT |
| | | TCAGTCAGATCAAGAGGAAATTGACTATGATGATACCATATCA |
| | | GTTGAAATGAAGAAGGAAGATTTTGACATTTATGATGAGGAT |
| | | GAAAATCAGAGCCCCCGCAGCTTTCAAAAGAAAACACGACAC |
| | | TATTTTATTGCTGCAGTGGAGAGGCTCTGGGATTATGGGATGA |
| | | GTAGCTCCCCACATGTTCTAAGAAACAGGGCTCAGAGTGGCA |
| | | GTGTCCCTCAGTTCAAGAAAGTTGTTTTCCAGGAATTTACTGA |
| | | TGGCTCCTTTACTCAGCCCTTATACCGTGGAGAACTAAATGAA |
| | | CATTTGGGACTCCTGGGGCCATATATAAGAGCAGAAGTTGAA |
| | | GATAATATCATGGTAACTTTCAGAAATCAGGCCTCTCGTCCCT |
| | | ATTCCTTCTATTCTAGCCTTATTTCTTATGAGGAAGATCAGAGG |
| | | CAAGGAGCAGAACCTAGAAAAAACTTTGTCAAGCCTAATGAA |
| | | ACCAAAACTTACTTTTGGAAAGTGCAACATCATATGGCACCCA |
| | | CTAAAGATGAGTTTGACTGCAAAGCCTGGGCTTATTTCTCTGA |
| | | TGTTGACCTGGAAAAAGATGTGCACTCAGGCCTGATTGGACCC |
| | | CTTCTGGTCTGCCACACTAACACACTGAACCCTGCTCATGGGA |
| | | GACAAGTGACAGTACAGGAATTTGCTCTGTTTTTCACCATCTTT |
| | | GATGAAACCAAAAGCTGGTACTTCACTGAAAATATGGAAAGA |
| | | AACTGCAGGGCTCCCTGCAATATCCAGATGGAAGATCCCACTT |
| | | TTAAAGAGAATTATCGCTTCCATGCAATCAATGGCTACATAAT |
| | | GGATACACTACCTGGCTTAGTAATGGCTCAGGATCAAAGGATT |
| | | CGATGGTATCTGCTCAGCATGGGCAGCAATGAAAACATCCATT |
| | | CTATTCATTTCAGTGGACATGTGTTCACTGTACGAAAAAAAGA |
| | | GGAGTATAAAATGGCACTGTACAATCTCTATCCAGGTGTTTTT |
| | | GAGACAGTGGAAATGTTACCATCCAAAGCTGGAATTTGGCGG |
| | | GTGGAATGCCTTATTGGCGAGCATCTACATGCTGGGATGAGCA |
| | | CACTTTTTCTGGTGTACAGCAATAAGTGTCAGACTCCCCTGGG |
| | | AATGGCTTCTGGACACATTAGAGATTTTCAGATTACAGCTTCA |
| | | GGACAATATGGACAGTGGGCCCCAAAGCTGGCCAGACTTCAT |
| | | TATTCCGGATCAATCAATGCCTGGAGCACCAAGGAGCCCTTTT |
| | | CTTGGATCAAGGTGGATCTGTTGGCACCAATGATTATTCACGG |
| | | CATCAAGACCCAGGGTGCCCGTCAGAAGTTCTCCAGCCTCTAC |
| | | ATCTCTCAGTTTATCATCATGTATAGTCTTGATGGGAAGAAGT |
| | | GGCAGACTTATCGAGGAAATTCCACTGGAACCTTAATGGTTTT |
| | | CTTTGGCAATGTGGATTCATCTGGGATAAAACACAATATTTTT |
| | | AACCCTCCAATTATTGCTCGATACATCCGTTTGCACCCAACTC |
| | | ATTATAGCATTCGCAGCACTCTTCGCATGGAGTTGATGGGCTG |
| | | TGATTTAAATAGTTGCAGCATGCCATTGGGAATGGAGAGTAAA |
| | | GCAATATCAGATGCACAGATTACTGCTTCATCCTACTTTACCA |
| | | ATATGTTTGCCACCTGGACTCCTTCAAAAGCTCGACTTCACCTC |
| | | CAAGGGAGGAGTAATGCCTGGCGACCTCAGGTGAATAATCCA |
| | | AAAGAGTGGCTGCAAGTGGACTTCCAGAAAACAATGAAAGTC |
| | | ACAGGAGTAACTACTCAGGGAGTAAAATCTCTGCTTACCAGCA |
| | | TGTATGTGAAGGAGTTCCTCATCTCCAGCAGTCAAGATGGCCA |
| | | TCAGTGGACTCTCTTTTTTCAGAATGGCAAAGTAAAGGTTTTTC |
| | | AGGGAAATCAAGACTCCTTCACACCTGTGGTGAACTCTCTAGA |
| | | CCCACCGTTACTGACTCGCTACCTTCGAATTCACCCCCAGAGT |
| | | TGGGTGCACCAGATTGCCCTGAGGATGGAGGTTCTGGGCTGCG |
| | | AGGCACAGGACCTCTACTGA |
| pBC0206 | MQIELSTCFFLCLLRF | ATGCAAATAGAGCTCTCCACCTGCTTCTTTCTGTGCCTTTTGCG |
| pBC0207 | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTVH | ATTCAACACCTCAGTCGTGTACAAAAAGACTCTGTTTGTAGAA |
| | LFNIAKPRPPWMGLL | TTCACGGTTCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGCCTTCATGCTGTTGGTGTATCCTACTGGAAAGCGTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTTTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNSSLPGLIGCH | CTCGGGCCTGGCCTAAAATGCACACAGTCAATGGTTATGTAAA |
| | RKSVYWHVIGMGTT | CAGTTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCGTCCTTGGAAATCTCGCCAATAACTTTCCTTACTGCTC |
| | HISSHQHDGMEAYVK | AAACACTCTTGATGGACCTTGGACAGTTTCTACTGTTTTGTCAT |
| | VDSCPEEPQLRMKNN | ATCTCTTCCCACCAACATGATGGCATGGAAGCCTATGTCAAAG |
| | EEAEDYDDDLTDSEM | TAGACAGCTGTCCAGAGGAACCCCAACTACGAATGAAAAATA |
| | DVVRFDDDNSPSFIQI | ATGAAGAAGCGGAAGATTATGATGATGATCTTACTGATTCTGA |
| | RSVAKKHPKTWVHYI | AATGGATGTGGTCAGGTTTGATGATGACAACTCTCCTTCCTTT |
| | AAEEEDWDYAPLVL | ATCCAAATTCGCTCAGTTGCCAAGAAGCATCCTAAAACTTGGG |
| | APDDRSYKSQYLNNG | TACATTACATTGCTGCTGAAGAGGAGGACTGGGACTATGCTCC |
| | PQRIGRKYKKVRFMA | CTTAGTCCTCGCCCCCGATGACAGAAGTTATAAAAGTCAATAT |
| | YTDETFKTREAIQHES | TTGAACAATGGCCCTCAGCGGATTGGTAGGAAGTACAAAAAA |
| | GILGPLLYGEVGDTL | GTCCGATTTATGGCATACACAGATGAAACCTTTAAGACTCGTG |
| | LIIFKNQASRPYNIYP | AAGCTATTCAGCATGAATCAGGAATCTTGGGACCTTTACTTTA |
| | HGITDVRPLYSRRLPK | TGGGGAAGTTGGAGACACACTGTTGATTATATTTAAGAATCAA |
| | GVKHLKDFPILPGEIF | GCAAGCAGACCATATAACATCTACCCTCACGGAATCACTGATG |
| | KYKWTVTVEDGPTK | TCCGTCCTTTGTATTCAAGGAGATTACCAAAAGGTGTAAAACA |
| | SDPRCLTRYYSSFVN | TTTGAAGGATTTTCCAATTCTGCCAGGAGAAATATTCAAATAT |
| | MERDLASGLIGPLLIC | AAATGGACAGTGACTGTAGAAGATGGGCCAACTAAATCAGAT |
| | YKESVDQRGNQIMSD | CCTCGGTGCCTGACCCGCTATTACTCTAGTTTCGTTAATATGGA |
| | KRNVILFSVFDENRS | GAGAGATCTAGCTTCAGGACTCATTGGCCCTCTCCTCATCTGC |
| | WYLTENIQRFLPNPA | TACAAAGAATCTGTAGATCAAAGAGGAAACCAGATAATGTCA |
| | GVQLEDPEFQASNIM | GACAAGAGGAATGTCATCCTGTTTTCTGTATTTGATGAGAACC |
| | HSINGYVFDSLQLSV | GAAGCTGGTACCTCACAGAGAATATACAACGCTTTCTCCCCAA |
| | CLHEVAYWYILSIGA | TCCAGCTGGAGTGCAGCTTGAGGATCCAGAGTTCCAAGCCTCC |
| | QTDFLSVFFSGYTFK | AACATCATGCACAGCATCAATGGCTATGTTTTTGATAGTTTGC |
| | HKMVYEDTLTLFPFS | AGTTGTCAGTTTGTTTGCATGAGGTGGCATACTGGTACATTCT |
| | GETVFMSMENPGLWI | AAGCATTGGAGCACAGACTGACTTCCTTTCTGTGTTCTTCTCTG |
| | LGCHNSDFRNRGMT | GATATACCTTCAAACACAAAATGGTCTATGAAGATACACTCAC |
| | ALLKVSSCDKNTGDY | CCTATTCCCATTCTCAGGAGAAACTGTGTTCATGTCGATGGAA |
| | YEDSYEDISAYLLSK | AACCCAGGTCTATGGATTCTGGGGTGCCACAACTCAGACTTTC |
| | NNAIEPRSFSQNGLGT | GGAACAGAGGCATGACCGCCTTACTGAAGGTTTCTAGTTGTGA |
| | SESATPESGPGSEPAT | CAAGAACACTGGTGATTATTACGAGGACAGTTATGAAGATATT |
| | SGSETPGTSESATPES | TCAGCATACTTGCTGAGTAAAAACAATGCCATTGAACCAAGA |
| | GPGSEPATSGSETPGT | AGCTTCTCCCAGAATGGTCTCGGTACCTCAGAGTCTGCTACCC |
| | SESATPESGPGTSTEP | CCGAGTCAGGGCCAGGATCAGAGCCAGCCACCTCCGGGTCTG |
| | SEGSAPGSPAGSPTST | AGACACCCGGGACTTCCGAGAGTGCCACCCCTGAGTCCGGAC |
| | EEGTSESATPESGPGS | CCGGGTCCGAGCCCGCCACTTCCGGCTCCGAAACTCCCGGCAC |
| | EPATSGSETPGTSESA | AAGCGAGAGCGCTACCCCAGAGTCAGGACCAGGAACATCTAC |
| | TPESGPGSPAGSPTST | AGAGCCCTCTGAAGGCTCCGCTCCAGGGTCCCCAGCCGGCAGT |
| | EEGSPAGSPTSTEEGT | CCCACTAGCACCGAGGAGGGAACCTCTGAAAGCGCCACACCC |
| | STEPSEGSAPGTSESA | GAATCAGGGCCAGGGTCTGAGCCTGCTACCAGCGGCAGCGAG |
| | TPESGPGTSESATPES | ACACCAGGCACCTCTGAGTCCGCCACACCAGAGTCCGGACCC |
| | GPGTSESATPESGPGS | GGATCTCCCGCTGGGAGCCCCACCTCCACTGAGGAGGGATCTC |
| | EPATSGSETPGSEPAT | CTGCTGGCTCTCCAACATCTACTGAGGAAGGTACCTCAACCGA |
| | SGSETPGSPAGSPTST | GCCATCCGAGGGATCAGCTCCCGGCACCTCAGAGTCGGCAAC |
| | EEGTSTEPSEGSAPGT | CCCGGAGTCTGGACCCGGAACTTCCGAAAGTGCCACACCAGA |
| | STEPSEGSAPGSEPAT | GTCCGGTCCCGGGACTTCAGAATCAGCAACACCCGAGTCCGGC |
| | SGSETPGTSESATPES | CCTGGGTCTGAACCCGCCACAAGTGGTAGTGAGACACCAGGA |
| | GPGTSTEPSEGSAPGL | TCAGAACCTGCTACCTCAGGGTCAGAGACACCCGGATCTCCGG |
| | PPVLKRHQREITRTTL | CAGGCTCACCAACCTCCACTGAGGAGGGCACCAGCACAGAAC |
| | QSDQEEIDYDDTISVE | CAAGCGAGGGCTCCGCACCCGGAACAAGCACTGAACCCAGTG |
| | MKKEDFDIYDEDENQ | AGGGTTCAGCACCCGGCTCTGAGCCGGCCACAAGTGGCAGTG |
| | SPRSFQKKTRHYFIAA | AGACACCCGGCACTTCAGAGAGTGCCACCCCCGAGAGTGGCC |
| | VERLWDYGMSSSPH | CAGGCACTAGTACCGAGCCCTCTGAAGGCAGTGCGCCAGGTCT |
| | VLRNRAQSGSVPQFK | CCCACCAGTCTTGAAACGCCATCAACGGGAAATAACTCGTACT |
| | KVVFQEFTDGSFTQP | ACTCTTCAGTCAGATCAAGAGGAAATTGACTATGATGATACCA |
| | LYRGELNEHLGLLGP | TATCAGTTGAAATGAAGAAGGAAGATTTTGACATTTATGATGA |
| | YIRAEVEDNIMVTFR | GGATGAAAATCAGAGCCCCCGCAGCTTTCAAAAGAAAACACG |
| | NQASRPYSFYSSLISY | ACACTATTTTATTGCTGCAGTGGAGAGGCTCTGGGATTATGGG |
| | EEDQRQGAEPRKNFV | ATGAGTAGCTCCCCACATGTTCTAAGAAACAGGGCTCAGAGTG |
| | KPNETKTYFWKVQH | GCAGTGTCCCTCAGTTCAAGAAAGTTGTTTTCCAGGAATTTAC |
| | HMAPTKDEFDCKAW | TGATGGCTCCTTTACTCAGCCCTTATACCGTGGAGAACTAAAT |
| | AYFSDVDLEKDVHSG | GAACATTTGGGACTCCTGGGGCCATATATAAGAGCAGAAGTT |
| | LIGPLLVCHTNTLNPA | GAAGATAATATCATGGTAACTTTCAGAAATCAGGCCTCTCGTC |
| | HGRQVTVQEFALFFTI | CCTATTCCTTCTATTCTAGCCTTATTTCTTATGAGGAAGATCAG |
| | FDETKSWYFTENMER | AGGCAAGGAGCAGAACCTAGAAAAAACTTTGTCAAGCCTAAT |
| | NCRAPCNIQMEDPTF | GAAACCAAAACTTACTTTTGGAAAGTGCAACATCATATGGCAC |
| | KENYRFHAINGYIMD | CCACTAAAGATGAGTTTGACTGCAAAGCCTGGGCTTATTTCTC |
| | TLPGLVMAQDQRIR | TGATGTTGACCTGGAAAAAGATGTGCACTCAGGCCTGATTGGA |
| | WYLLSMGSNENIHSI | CCCCTTCTGGTCTGCCACACTAACACACTGAACCCTGCTCATG |
| | HFSGHVFTVRKKEEY | GGAGACAAGTGACAGTACAGGAATTTGCTCTGTTTTTCACCAT |
| | KMALYNLYPGVFET | CTTTGATGAAACCAAAAGCTGGTACTTCACTGAAAATATGGAA |
| | VEMLPSKAGIWRVEC | AGAAACTGCAGGGCTCCCTGCAATATCCAGATGGAAGATCCC |
| | LIGEHLHAGMSTLFL | ACTTTTAAAGAGAATTATCGCTTCCATGCAATCAATGGCTACA |
| | VYSNKCQTPLGMAS | TAATGGATACACTACCTGGCTTAGTAATGGCTCAGGATCAAAG |
| | GHIRDFQITASGQYG | GATTCGATGGTATCTGCTCAGCATGGGCAGCAATGAAAACATC |
| | QWAPKLARLHYSGSI | CATTCTATTCATTTCAGTGGACATGTGTTCACTGTACGAAAAA |
| | NAWSTKEPFSWIKVD | AAGAGGAGTATAAAATGGCACTGTACAATCTCTATCCAGGTGT |
| | LLAPMIIHGIKTQGAR | TTTTGAGACAGTGGAAATGTTACCATCCAAAGCTGGAATTTGG |
| | QKFSSLYISQFIIMYSL | CGGGTGGAATGCCTTATTGGCGAGCATCTACATGCTGGGATGA |
| | DGKKWQTYRGNSTG | GCACACTTTTTCTGGTGTACAGCAATAAGTGTCAGACTCCCCT |
| | TLMVFFGNVDSSGIK | GGGAATGGCTTCTGGACACATTAGAGATTTTCAGATTACAGCT |
| | HNIFNPPIIARYIRLHP | TCAGGACAATATGGACAGTGGGCCCCAAAGCTGGCCAGACTT |
| | THYSIRSTLRMELMG | CATTATTCCGGATCAATCAATGCCTGGAGCACCAAGGAGCCCT |
| | CDLNSCSMPLGMESK | TTTCTTGGATCAAGGTGGATCTGTTGGCACCAATGATTATTCA |
| | AISDAQITASSYFTNM | CGGCATCAAGACCCAGGGTGCCCGTCAGAAGTTCTCCAGCCTC |
| | FATWTPSKARLHLQG | TACATCTCTCAGTTTATCATCATGTATAGTCTTGATGGGAAGA |
| | RSNAWRPQVNNPKE | AGTGGCAGACTTATCGAGGAAATTCCACTGGAACCTTAATGGT |
| | WLQVDFQKTMKVTG | TTTCTTTGGCAATGTGGATTCATCTGGGATAAAACACAATATT |
| | VTTQGVKSLLTSMYV | TTTAACCCTCCAATTATTGCTCGATACATCCGTTTGCACCCAAC |
| | KEFLISSSQDGHQWT | TCATTATAGCATTCGCAGCACTCTTCGCATGGAGTTGATGGGC |
| | LFFQNGKVKVFQGN | TGTGATTTAAATAGTTGCAGCATGCCATTGGGAATGGAGAGTA |
| | QDSFTPVVNSLDPPLL | AAGCAATATCAGATGCACAGATTACTGCTTCATCCTACTTTAC |
| | TRYLRIHPQSWVHQI | CAATATGTTTGCCACCTGGACTCCTTCAAAAGCTCGACTTCAC |
| | ALRMEVLGCEAQDLY | CTCCAAGGGAGGAGTAATGCCTGGCGACCTCAGGTGAATAAT |
| | | CCAAAAGAGTGGCTGCAAGTGGACTTCCAGAAAACAATGAAA |
| | | GTCACAGGAGTAACTACTCAGGGAGTAAAATCTCTGCTTACCA |
| | | GCATGTATGTGAAGGAGTTCCTCATCTCCAGCAGTCAAGATGG |
| | | CCATCAGTGGACTCTCTTTTTTCAGAATGGCAAAGTAAAGGTT |
| | | TTTCAGGGAAATCAAGACTCCTTCACACCTGTGGTGAACTCTC |
| | | TAGACCCACCGTTACTGACTCGCTACCTTCGAATTCACCCCCA |
| | | GAGTTGGGTGCACCAGATTGCCCTGAGGATGGAGGTTCTGGGC |
| | | TGCGAGGCACAGGACCTCTACTGA |
| pBC0208 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKGAPGSPAGSPTST | TCAGATTACAGCTTCAGGACAATATGGACAGTGGGCCCCAAA |
| | EEGTSESATPESGPGS | GCTGGCCAGACTTCATTATTCCGGATCAATCAATGCCTGGAGC |
| | EPATSGSETPASSFSS | ACCAAGGAGCCCTTTTCTTGGATCAAGGTGGATCTGTTGGCAC |
| | LYISQFIIMYSLDGKK | CAATGATTATTCACGGCATCAAGACCCAGGGTGCCCGTCAGAA |
| | WQTYRGNSTGTLMV | GGGCGCGCCAGGTTCTCCTGCTGGCTCCCCCACCTCAACAGAA |
| | FFGNVDSSGIKHNIFN | GAGGGGACAAGCGAAAGCGCTACGCCTGAGAGTGGCCCTGGC |
| | PPIIARYIRLHPTHYSI | TCTGAGCCAGCCACCTCCGGCTCTGAAACCCCTGCCTCGAGCT |
| | RSTLRMELMGCDLNS | TCTCCAGCCTCTACATCTCTCAGTTTATCATCATGTATAGTCTT |
| | CSMPLGMESKAISDA | GATGGGAAGAAGTGGCAGACTTATCGAGGAAATTCCACTGGA |
| | QITASSYFTNMFATW | ACCTTAATGGTCTTCTTTGGCAATGTGGATTCATCTGGGATAA |
| | SPSKARLHLQGRSNA | AACACAATATTTTTAACCCTCCAATTATTGCTCGATACATCCGT |
| | WRPQVNNPKEWLQV | TTGCACCCAACTCATTATAGCATTCGCAGCACTCTTCGCATGG |
| | DFQKTMKVTGVTTQ | AGTTGATGGGCTGTGATTTAAATAGTTGCAGCATGCCATTGGG |
| | GVKSLLTSMYVKEFL | AATGGAGAGTAAAGCAATATCAGATGCACAGATTACTGCTTC |
| | ISSSQDGHQWTLFFQ | ATCCTACTTTACCAATATGTTTGCCACCTGGTCTCCTTCAAAAG |
| | NGKVKVFQGNQDSF | CTCGACTTCACCTCCAAGGGAGGAGTAATGCCTGGAGACCTCA |
| | TPVVNSLDPPLLTRYL | GGTGAATAATCCAAAAGAGTGGCTGCAAGTGGACTTCCAGAA |
| | RIHPQSWVHQIALRM | GACAATGAAAGTCACAGGAGTAACTACTCAGGGAGTAAAATC |
| | EVLGCEAQDLYGAGS | TCTGCTTACCAGCATGTATGTGAAGGAGTTCCTCATCTCCAGC |
| | PGAETAEQKLISEEDL | AGTCAAGATGGCCATCAGTGGACTCTCTTTTTTCAGAATGGCA |
| | SPATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0180 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKGAPGSPAGSPT | GCTGGCCAGACTTCATTATTCCGGATCAATCAATGCCTGGAGC |
| | STEEGTSESATPESGP | ACCAAGGAGCCCTTTTCTTGGATCAAGGTGGATCTGTTGGCAC |
| | GSEPATSGSETPASSW | CAATGATTATTCACGGCATCAAGACCCAGGGTGCCCGTCAGAA |
| | QTYRGNSTGTLMVFF | GTTCTCCAGCCTCTACATCTCTCAGTTTATCATCATGTATAGTC |
| | GNVDSSGIKHNIFNPP | TTGATGGGAAGAAGGGCGCGCCAGGTTCTCCTGCTGGCTCCCC |
| | IIARYIRLHPTHYSIRS | CACCTCAACAGAAGAGGGGACAAGCGAAAGCGCTACGCCTGA |
| | TLRMELMGCDLNSCS | GAGTGGCCCTGGCTCTGAGCCAGCCACCTCCGGCTCTGAAACC |
| | MPLGMESKAISDAQI | CCTGCCTCGAGCTGGCAGACTTATCGAGGAAATTCCACTGGAA |
| | TASSYFTNMFATWSP | CCTTAATGGTCTTCTTTGGCAATGTGGATTCATCTGGGATAAA |
| | SKARLHLQGRSNAW | ACACAATATTTTTAACCCTCCAATTATTGCTCGATACATCCGTT |
| | RPQVNNPKEWLQVD | TGCACCCAACTCATTATAGCATTCGCAGCACTCTTCGCATGGA |
| | FQKTMKVTGVTTQG | GTTGATGGGCTGTGATTTAAATAGTTGCAGCATGCCATTGGGA |
| | VKSLLTSMYVKEFLIS | ATGGAGAGTAAAGCAATATCAGATGCACAGATTACTGCTTCAT |
| | SSQDGHQWTLFFQNG | CCTACTTTACCAATATGTTTGCCACCTGGTCTCCTTCAAAAGCT |
| | KVKVFQGNQDSFTPV | CGACTTCACCTCCAAGGGAGGAGTAATGCCTGGAGACCTCAG |
| | VNSLDPPLLTRYLRIH | GTGAATAATCCAAAAGAGTGGCTGCAAGTGGACTTCCAGAAG |
| | PQSWVHQIALRMEVL | ACAATGAAAGTCACAGGAGTAACTACTCAGGGAGTAAAATCT |
| | GCEAQDLYGAGSPG | CTGCTTACCAGCATGTATGTGAAGGAGTTCCTCATCTCCAGCA |
| | AETAEQKLISEEDLSP | GTCAAGATGGCCATCAGTGGACTCTCTTTTTTCAGAATGGCAA |
| | ATG | AGTAAAGGTTTTTCAGGGAAATCAAGACTCCTTCACACCTGTG |
| | | GTGAACTCTCTAGACCCACCGTTACTGACTCGCTACCTTCGAA |
| | | TTCACCCCCAGAGTTGGGTGCACCAGATTGCCCTGAGGATGGA |
| | | GGTTCTGGGCTGCGAGGCACAGGACCTCTACGGCGCCGGATC |
| | | ACCTGGGGCCGAAACGGCCGAACAAAAACTCATCTCAGAAGA |
| | | GGATCTGTCACCTGCAACCGGTTGA |
| pBC0142 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKWQTYRGNSTG | GCTGGCCAGACTTCATTATTCCGGATCAATCAATGCCTGGAGC |
| | APGSPAGSPTSTEEGT | ACCAAGGAGCCCTTTTCTTGGATCAAGGTGGATCTGTTGGCAC |
| | SESATPESGPGSEPAT | CAATGATTATTCACGGCATCAAGACCCAGGGTGCCCGTCAGAA |
| | SGSETPASSGTLMVFF | GTTCTCCAGCCTCTACATCTCTCAGTTTATCATCATGTATAGTC |
| | GNVDSSGIKHNIFNPP | TTGATGGGAAGAAGTGGCAGACTTATCGAGGAAATTCCACTG |
| | IIARYIRLHPTHYSIRS | GCGCGCCAGGTTCTCCTGCTGGCTCCCCCACCTCAACAGAAGA |
| | TLRMELMGCDLNSCS | GGGGACAAGCGAAAGCGCTACGCCTGAGAGTGGCCCTGGCTC |
| | MPLGMESKAISDAQI | TGAGCCAGCCACCTCCGGCTCTGAAACCCCTGCCTCGAGCGGA |
| | TASSYFTNMFATWSP | ACCTTAATGGTCTTCTTTGGCAATGTGGATTCATCTGGGATAA |
| | SKARLHLQGRSNAW | AACACAATATTTTTAACCCTCCAATTATTGCTCGATACATCCGT |
| | RPQVNNPKEWLQVD | TTGCACCCAACTCATTATAGCATTCGCAGCACTCTTCGCATGG |
| | FQKTMKVTGVTTQG | AGTTGATGGGCTGTGATTTAAATAGTTGCAGCATGCCATTGGG |
| | VKSLLTSMYVKEFLIS | AATGGAGAGTAAAGCAATATCAGATGCACAGATTACTGCTTC |
| | SSQDGHQWTLFFQNG | ATCCTACTTTACCAATATGTTTGCCACCTGGTCTCCTTCAAAAG |
| | KVKVFQGNQDSFTPV | CTCGACTTCACCTCCAAGGGAGGAGTAATGCCTGGAGACCTCA |
| | VNSLDPPLLTRYLRIH | GGTGAATAATCCAAAAGAGTGGCTGCAAGTGGACTTCCAGAA |
| | PQSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0143 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKWQTYRGNSTG | GCTGGCCAGACTTCATTATTCCGGATCAATCAATGCCTGGAGC |
| | TLMVFFGNVDSSGIK | ACCAAGGAGCCCTTTTCTTGGATCAAGGTGGATCTGTTGGCAC |
| | HNIFNPPIIARYIRLHP | CAATGATTATTCACGGCATCAAGACCCAGGGTGCCCGTCAGAA |
| | THYSIRSTLRMELMG | GTTCTCCAGCCTCTACATCTCTCAGTTTATCATCATGTATAGTC |
| | CDLGAPGSPAGSPTST | TTGATGGGAAGAAGTGGCAGACTTATCGAGGAAATTCCACTG |
| | EEGTSESATPESGPGS | GAACCTTAATGGTCTTCTTTGGCAATGTGGATTCATCTGGGAT |
| | EPATSGSETPASSNSC | AAAACACAATATTTTTAACCCTCCAATTATTGCTCGATACATC |
| | SMPLGMESKAISDAQ | CGTTTGCACCCAACTCATTATAGCATTCGCAGCACTCTTCGCAT |
| | ITASSYFTNMFATWS | GGAGTTGATGGGCTGTGATTTAGGCGCGCCAGGTTCTCCTGCT |
| | PSKARLHLQGRSNA | GGCTCCCCCACCTCAACAGAAGAGGGGACAAGCGAAAGCGCT |
| | WRPQVNNPKEWLQV | ACGCCTGAGAGTGGCCCTGGCTCTGAGCCAGCCACCTCCGGCT |
| | DFQKTMKVTGVTTQ | CTGAAACCCCTGCCTCGAGCAATAGTTGCAGCATGCCATTGGG |
| | GVKSLLTSMYVKEFL | AATGGAGAGTAAAGCAATATCAGATGCACAGATTACTGCTTC |
| | ISSSQDGHQWTLFFQ | ATCCTACTTTACCAATATGTTTGCCACCTGGTCTCCTTCAAAAG |
| | NGKVKVFQGNQDSF | CTCGACTTCACCTCCAAGGGAGGAGTAATGCCTGGAGACCTCA |
| | TPVVNSLDPPLLTRYL | GGTGAATAATCCAAAAGAGTGGCTGCAAGTGGACTTCCAGAA |
| | RIHPQSWVHQIALRM | GACAATGAAAGTCACAGGAGTAACTACTCAGGGAGTAAAATC |
| | EVLGCEAQDLYGAGS | TCTGCTTACCAGCATGTATGTGAAGGAGTTCCTCATCTCCAGC |
| | PGAETAEQKLISEEDL | AGTCAAGATGGCCATCAGTGGACTCTCTTTTTTCAGAATGGCA |
| | SPATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0181 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKWQTYRGNSTG | GCTGGCCAGACTTCATTATTCCGGATCAATCAATGCCTGGAGC |
| | TLMVFFGNVDSSGIK | ACCAAGGAGCCCTTTTCTTGGATCAAGGTGGATCTGTTGGCAC |
| | HNIFNPPIIARYIRLHP | CAATGATTATTCACGGCATCAAGACCCAGGGTGCCCGTCAGAA |
| | THYSIRSTLRMELMG | GTTCTCCAGCCTCTACATCTCTCAGTTTATCATCATGTATAGTC |
| | CDLNSCSMPLGMESK | TTGATGGGAAGAAGTGGCAGACTTATCGAGGAAATTCCACTG |
| | AISDAGAPGSPAGSPT | GAACCTTAATGGTCTTCTTTGGCAATGTGGATTCATCTGGGAT |
| | STEEGTSESATPESGP | AAAACACAATATTTTTAACCCTCCAATTATTGCTCGATACATC |
| | GSEPATSGSETPASSQ | CGTTTGCACCCAACTCATTATAGCATTCGCAGCACTCTTCGCAT |
| | ITASSYFTNMFATWS | GGAGTTGATGGGCTGTGATTTAAATAGTTGCAGCATGCCATTG |
| | PSKARLHLQGRSNA | GGAATGGAGAGTAAAGCAATATCAGATGCAGGCGCGCCAGGT |
| | WRPQVNNPKEWLQV | TCTCCTGCTGGCTCCCCCACCTCAACAGAAGAGGGGACAAGCG |
| | DFQKTMKVTGVTTQ | AAAGCGCTACGCCTGAGAGTGGCCCTGGCTCTGAGCCAGCCA |
| | GVKSLLTSMYVKEFL | CCTCCGGCTCTGAAACCCCTGCCTCGAGCCAGATTACTGCTTC |
| | ISSSQDGHQWTLFFQ | ATCCTACTTTACCAATATGTTTGCCACCTGGTCTCCTTCAAAAG |
| | NGKVKVFQGNQDSF | CTCGACTTCACCTCCAAGGGAGGAGTAATGCCTGGAGACCTCA |
| | TPVVNSLDPPLLTRYL | GGTGAATAATCCAAAAGAGTGGCTGCAAGTGGACTTCCAGAA |
| | RIHPQSWVHQIALRM | GACAATGAAAGTCACAGGAGTAACTACTCAGGGAGTAAAATC |
| | EVLGCEAQDLYGAGS | TCTGCTTACCAGCATGTATGTGAAGGAGTTCCTCATCTCCAGC |
| | PGAETAEQKLISEEDL | AGTCAAGATGGCCATCAGTGGACTCTCTTTTTTCAGAATGGCA |
| | SPATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0182 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKWQTYRGNSTG | GCTGGCCAGACTTCATTATTCCGGATCAATCAATGCCTGGAGC |
| | TLMVFFGNVDSSGIK | ACCAAGGAGCCCTTTTCTTGGATCAAGGTGGATCTGTTGGCAC |
| | HNIFNPPIIARYIRLHP | CAATGATTATTCACGGCATCAAGACCCAGGGTGCCCGTCAGAA |
| | THYSIRSTLRMELMG | GTTCTCCAGCCTCTACATCTCTCAGTTTATCATCATGTATAGTC |
| | CDLNSCSMPLGMESK | TTGATGGGAAGAAGTGGCAGACTTATCGAGGAAATTCCACTG |
| | AISDAQITASSYFTNM | GAACCTTAATGGTCTTCTTTGGCAATGTGGATTCATCTGGGAT |
| | FATWSPSKARLHLQG | AAAACACAATATTTTTAACCCTCCAATTATTGCTCGATACATC |
| | RSNAWRPQVNNPKG | CGTTTGCACCCAACTCATTATAGCATTCGCAGCACTCTTCGCAT |
| | APGSPAGSPTSTEEGT | GGAGTTGATGGGCTGTGATTTAAATAGTTGCAGCATGCCATTG |
| | SESATPESGPGSEPAT | GGAATGGAGAGTAAAGCAATATCAGATGCACAGATTACTGCT |
| | SGSETPASSEWLQVD | TCATCCTACTTTACCAATATGTTTGCCACCTGGTCTCCTTCAAA |
| | FQKTMKVTGVTTQG | AGCTCGACTTCACCTCCAAGGGAGGAGTAATGCCTGGAGACCT |
| | VKSLLTSMYVKEFLIS | CAGGTGAATAATCCAAAAGGCGCGCCAGGTTCTCCTGCTGGCT |
| | SSQDGHQWTLFFQNG | CCCCCACCTCAACAGAAGAGGGGACAAGCGAAAGCGCTACGC |
| | KVKVFQGNQDSFTPV | CTGAGAGTGGCCCTGGCTCTGAGCCAGCCACCTCCGGCTCTGA |
| | VNSLDPPLLTRYLRIH | AACCCCTGCCTCGAGCGAGTGGCTGCAAGTGGACTTCCAGAA |
| | PQSWVHQIALRMEVL | GACAATGAAAGTCACAGGAGTAACTACTCAGGGAGTAAAATC |
| | GCEAQDLYGAGSPG | TCTGCTTACCAGCATGTATGTGAAGGAGTTCCTCATCTCCAGC |
| | AETAEQKLISEEDLSP | AGTCAAGATGGCCATCAGTGGACTCTCTTTTTTCAGAATGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0144 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKWQTYRGNSTG | GCTGGCCAGACTTCATTATTCCGGATCAATCAATGCCTGGAGC |
| | TLMVFFGNVDSSGIK | ACCAAGGAGCCCTTTTCTTGGATCAAGGTGGATCTGTTGGCAC |
| | HNIFNPPIIARYIRLHP | CAATGATTATTCACGGCATCAAGACCCAGGGTGCCCGTCAGAA |
| | THYSIRSTLRMELMG | GTTCTCCAGCCTCTACATCTCTCAGTTTATCATCATGTATAGTC |
| | CDLNSCSMPLGMESK | TTGATGGGAAGAAGTGGCAGACTTATCGAGGAAATTCCACTG |
| | AISDAQITASSYFTNM | GAACCTTAATGGTCTTCTTTGGCAATGTGGATTCATCTGGGAT |
| | FATWSPSKARLHLQG | AAAACACAATATTTTTAACCCTCCAATTATTGCTCGATACATC |
| | RSNAWRPQVNNPKE | CGTTTGCACCCAACTCATTATAGCATTCGCAGCACTCTTCGCAT |
| | WLQVDFQKTMKVTG | GGAGTTGATGGGCTGTGATTTAAATAGTTGCAGCATGCCATTG |
| | VTTQGVKSLLTSMYV | GGAATGGAGAGTAAAGCAATATCAGATGCACAGATTACTGCT |
| | KEFLISSSQDGHQWT | TCATCCTACTTTACCAATATGTTTGCCACCTGGTCTCCTTCAAA |
| | LFFQNGAPGSPAGSPT | AGCTCGACTTCACCTCCAAGGGAGGAGTAATGCCTGGAGACCT |
| | STEEGTSESATPESGP | CAGGTGAATAATCCAAAAGAGTGGCTGCAAGTGGACTTCCAG |
| | GSEPATSGSETPASSG | AAGACAATGAAAGTCACAGGAGTAACTACTCAGGGAGTAAAA |
| | KVKVFQGNQDSFTPV | TCTCTGCTTACCAGCATGTATGTGAAGGAGTTCCTCATCTCCA |
| | VNSLDPPLLTRYLRIH | GCAGTCAAGATGGCCATCAGTGGACTCTCTTTTTTCAGAATGG |
| | PQSWVHQIALRMEVL | CGCGCCAGGTTCTCCTGCTGGCTCCCCCACCTCAACAGAAGAG |
| | GCEAQDLYGAGSPG | GGGACAAGCGAAAGCGCTACGCCTGAGAGTGGCCCTGGCTCT |
| | AETAEQKLISEEDLSP | GAGCCAGCCACCTCCGGCTCTGAAACCCCTGCCTCGAGCGGCA |
| | ATG | AAGTAAAGGTTTTTCAGGGAAATCAAGACTCCTTCACACCTGT |
| | | GGTGAACTCTCTAGACCCACCGTTACTGACTCGCTACCTTCGA |
| | | ATTCACCCCCAGAGTTGGGTGCACCAGATTGCCCTGAGGATGG |
| | | AGGTTCTGGGCTGCGAGGCACAGGACCTCTACGGCGCCGGAT |
| | | CACCTGGGGCCGAAACGGCCGAACAAAAACTCATCTCAGAAG |
| | | AGGATCTGTCACCTGCAACCGGTTGA |
| pBC0145 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKWQTYRGNSTG | GCTGGCCAGACTTCATTATTCCGGATCAATCAATGCCTGGAGC |
| | TLMVFFGNVDSSGIK | ACCAAGGAGCCCTTTTCTTGGATCAAGGTGGATCTGTTGGCAC |
| | HNIFNPPIIARYIRLHP | CAATGATTATTCACGGCATCAAGACCCAGGGTGCCCGTCAGAA |
| | THYSIRSTLRMELMG | GTTCTCCAGCCTCTACATCTCTCAGTTTATCATCATGTATAGTC |
| | CDLNSCSMPLGMESK | TTGATGGGAAGAAGTGGCAGACTTATCGAGGAAATTCCACTG |
| | AISDAQITASSYFTNM | GAACCTTAATGGTCTTCTTTGGCAATGTGGATTCATCTGGGAT |
| | FATWSPSKARLHLQG | AAAACACAATATTTTTAACCCTCCAATTATTGCTCGATACATC |
| | RSNAWRPQVNNPKE | CGTTTGCACCCAACTCATTATAGCATTCGCAGCACTCTTCGCAT |
| | WLQVDFQKTMKVTG | GGAGTTGATGGGCTGTGATTTAAATAGTTGCAGCATGCCATTG |
| | VTTQGVKSLLTSMYV | GGAATGGAGAGTAAAGCAATATCAGATGCACAGATTACTGCT |
| | KEFLISSSQDGHQWT | TCATCCTACTTTACCAATATGTTTGCCACCTGGTCTCCTTCAAA |
| | LFFQNGKVKVFQGN | AGCTCGACTTCACCTCCAAGGGAGGAGTAATGCCTGGAGACCT |
| | QDSFTPVVNSLDPPLL | CAGGTGAATAATCCAAAAGAGTGGCTGCAAGTGGACTTCCAG |
| | TRYLRIHPQSWVHQI | AAGACAATGAAAGTCACAGGAGTAACTACTCAGGGAGTAAAA |
| | ALRMEVLGCEAQDL | TCTCTGCTTACCAGCATGTATGTGAAGGAGTTCCTCATCTCCA |
| | YGAGSPGAETAGTSE | GCAGTCAAGATGGCCATCAGTGGACTCTCTTTTTTCAGAATGG |
| | SATPESGPGSEPATSG | CAAAGTAAAGGTTTTTCAGGGAAATCAAGACTCCTTCACACCT |
| | SETPGTSESATPESGP | GTGGTGAACTCTCTAGACCCACCGTTACTGACTCGCTACCTTC |
| | GSEPATSGSETPGTSE | GAATTCACCCCCAGAGTTGGGTGCACCAGATTGCCCTGAGGAT |
| | SATPESGPGTSTEPSE | GGAGGTTCTGGGCTGCGAGGCACAGGACCTCTACGGCGCCGG |
| | GSAPGSPAGSPTSTEE | ATCACCTGGGGCCGAAACGGCCGGTACCTCAGAGTCTGCTACC |
| | GTSESATPESGPGSEP | CCCGAGTCAGGGCCAGGATCAGAGCCAGCCACCTCCGGGTCT |
| | ATSGSETPGTSESATP | GAGACACCCGGGACTTCCGAGAGTGCCACCCCTGAGTCCGGA |
| | ESGPGSPAGSPTSTEE | CCCGGGTCCGAGCCCGCCACTTCCGGCTCCGAAACTCCCGGCA |
| | GSPAGSPTSTEEGTST | CAAGCGAGAGCGCTACCCCAGAGTCAGGACCAGGAACATCTA |
| | EPSEGSAPGTSESATP | CAGAGCCCTCTGAAGGCTCCGCTCCAGGGTCCCCAGCCGGCAG |
| | ESGPGTSESATPESGP | TCCCACTAGCACCGAGGAGGGAACCTCTGAAAGCGCCACACC |
| | GTSESATPESGPGSEP | CGAATCAGGGCCAGGGTCTGAGCCTGCTACCAGCGGCAGCGA |
| | ATSGSETPGSEPATSG | GACACCAGGCACCTCTGAGTCCGCCACACCAGAGTCCGGACC |
| | SETPGSPAGSPTSTEE | CGGATCTCCCGCTGGGAGCCCCACCTCCACTGAGGAGGGATCT |
| | GTSTEPSEGSAPGTST | CCTGCTGGCTCTCCAACATCTACTGAGGAAGGTACCTCAACCG |
| | EPSEGSAPGSEPATSG | AGCCATCCGAGGGATCAGCTCCCGGCACCTCAGAGTCGGCAA |
| | SETPGTSESATPESGP | CCCCGGAGTCTGGACCCGGAACTTCCGAAAGTGCCACACCAG |
| | GTSTEPSEGSAPGAET | AGTCCGGTCCCGGGACTTCAGAATCAGCAACACCCGAGTCCG |
| | AEQKLISEEDLSPATG | GCCCTGGGTCTGAACCCGCCACAAGTGGTAGTGAGACACCAG |
| | | GATCAGAACCTGCTACCTCAGGGTCAGAGACACCCGGATCTCC |
| | | GGCAGGCTCACCAACCTCCACTGAGGAGGGCACCAGCACAGA |
| | | ACCAAGCGAGGGCTCCGCACCCGGAACAAGCACTGAACCCAG |
| | | TGAGGGTTCAGCACCCGGCTCTGAGCCGGCCACAAGTGGCAG |
| | | TGAGACACCCGGCACTTCAGAGAGTGCCACCCCCGAGAGTGG |
| | | CCCAGGCACTAGTACCGAGCCCTCTGAAGGCAGTGCGCCAGG |
| | | GGCCGAAACGGCCGAACAAAAACTCATCTCAGAAGAGGATCT |
| | | GTCACCTGCAACCGGTTGA |
| pBC0146 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNPPVL | GGAACAGAGGCATGACCGCCTTACTGAAGGTTTCTAGTTGTGA |
| | KRHQREITRTTLQSD | CAAGAACACTGGTGATTATTACGAGGACAGTTATGAAGATATT |
| | QEEIDYDDTISVEMK | TCAGCATACTTGCTGAGTAAAAACAATGCCATTGAACCAAGA |
| | KEDFDIYDEDENQSP | AGCTTCTCTCAAAACCCACCAGTCTTGAAACGCCATCAACGGG |
| | RSFQKKTRHYFIAAV | AAATAACTCGTACTACTCTTCAGTCAGATCAAGAGGAAATCGA |
| | ERLWDYGMSSSPHVL | TTATGATGATACCATATCAGTTGAAATGAAGAAGGAAGATTTT |
| | RNRAQSGSVPQFKKV | GACATTTATGATGAGGATGAAAATCAGAGCCCCCGCAGCTTTC |
| | VFQEFTDGSFTQPLY | AAAAGAAAACACGACACTATTTTATTGCTGCAGTGGAGAGGC |
| | RGELNEHLGLLGPYI | TCTGGGATTATGGGATGAGTAGCTCCCCACATGTTCTAAGAAA |
| | RAEVEDNIMVTFRNQ | CAGGGCTCAGAGTGGCAGTGTCCCTCAGTTCAAGAAAGTTGTT |
| | ASRPYSFYSSLISYEE | TTCCAGGAATTTACTGATGGCTCCTTTACTCAGCCCTTATACCG |
| | DQRQGAEPRKNFVKP | TGGAGAACTAAATGAACATTTGGGACTCCTGGGGCCATATATA |
| | NETKTYFWKVQHHM | AGAGCAGAAGTTGAAGATAATATCATGGTAACTTTCAGAAAT |
| | APTKDEFDCKAWAY | CAGGCCTCTCGTCCCTATTCCTTCTATTCTAGCCTTATTTCTTAT |
| | FSDVDLEKDVHSGLI | GAGGAAGATCAGAGGCAAGGAGCAGAACCTAGAAAAAACTTT |
| | GPLLVCHTNTLNPAH | GTCAAGCCTAATGAAACCAAAACTTACTTTTGGAAAGTGCAAC |
| | GRQVTVQEFALFFTIF | ATCATATGGCACCCACTAAAGATGAGTTTGACTGCAAAGCCTG |
| | DETKSWYFTENMER | GGCTTATTTCTCTGATGTTGACCTGGAAAAAGATGTGCACTCA |
| | NCRAPCNIQMEDPTF | GGCCTGATTGGACCCCTTCTGGTCTGCCACACTAACACACTGA |
| | KENYRFHAINGYIMD | ACCCTGCTCATGGGAGACAAGTGACAGTACAGGAATTTGCTCT |
| | TLPGLVMAQDQRIR | GTTTTTCACCATCTTTGATGAGACCAAAAGCTGGTACTTCACT |
| | WYLLSMGSNENIHSI | GAAAATATGGAAAGAAACTGCAGGGCTCCCTGCAATATCCAG |
| | HFSGHVFTVRKKEEY | ATGGAAGATCCCACTTTTAAAGAGAATTATCGCTTCCATGCAA |
| | KMALYNLYPGVFET | TCAATGGCTACATAATGGATACACTACCTGGCTTAGTAATGGC |
| | VEMLPSKAGIWRVEC | TCAGGATCAAAGGATTCGATGGTATCTGCTCAGCATGGGCAGC |
| | LIGEHLHAGMSTLFL | AATGAAAACATCCATTCTATTCATTTCAGTGGACATGTGTTCA |
| | VYSNKCQTPLGMAS | CTGTACGAAAAAAAGAGGAGTATAAAATGGCACTGTACAATC |
| | GHIRDFQITASGQYG | TCTATCCAGGTGTTTTTGAGACAGTGGAAATGTTACCATCCAA |
| | QWAPKLARLHYSGSI | AGCTGGAATTTGGCGGGTGGAATGCCTTATTGGCGAGCATCTA |
| | NAWSTKEPFSWIKVD | CATGCTGGGATGAGCACACTTTTTCTGGTGTACAGCAATAAGT |
| | LLAPMIIHGIKTQGAR | GTCAGACTCCCCTGGGAATGGCTTCTGGACACATTAGAGATTT |
| | QKFSSLYISQFIIMYSL | TCAGATTACAGCTTCAGGACAATATGGACAGTGGGCCCCAAA |
| | DGKKWQTYRGNSTG | GCTGGCCAGACTTCATTATTCCGGATCAATCAATGCCTGGAGC |
| | TLMVFFGNVDSSGIK | ACCAAGGAGCCCTTTTCTTGGATCAAGGTGGATCTGTTGGCAC |
| | HNIFNPPIIARYIRLHP | CAATGATTATTCACGGCATCAAGACCCAGGGTGCCCGTCAGAA |
| | THYSIRSTLRMELMG | GTTCTCCAGCCTCTACATCTCTCAGTTTATCATCATGTATAGTC |
| | CDLNSCSMPLGMESK | TTGATGGGAAGAAGTGGCAGACTTATCGAGGAAATTCCACTG |
| | AISDAQITASSYFTNM | GAACCTTAATGGTCTTCTTTGGCAATGTGGATTCATCTGGGAT |
| | FATWSPSKARLHLQG | AAAACACAATATTTTTAACCCTCCAATTATTGCTCGATACATC |
| | RSNAWRPQVNNPKE | CGTTTGCACCCAACTCATTATAGCATTCGCAGCACTCTTCGCAT |
| | WLQVDFQKTMKVTG | GGAGTTGATGGGCTGTGATTTAAATAGTTGCAGCATGCCATTG |
| | VTTQGVKSLLTSMYV | GGAATGGAGAGTAAAGCAATATCAGATGCACAGATTACTGCT |
| | KEFLISSSQDGHQWT | TCATCCTACTTTACCAATATGTTTGCCACCTGGTCTCCTTCAAA |
| | LFFQNGKVKVFQGN | AGCTCGACTTCACCTCCAAGGGAGGAGTAATGCCTGGAGACCT |
| | QDSFTPVVNSLDPPLL | CAGGTGAATAATCCAAAAGAGTGGCTGCAAGTGGACTTCCAG |
| | TRYLRIHPQSWVHQI | AAGACAATGAAAGTCACAGGAGTAACTACTCAGGGAGTAAAA |
| | ALRMEVLGCEAQDL | TCTCTGCTTACCAGCATGTATGTGAAGGAGTTCCTCATCTCCA |
| | YGAGSPGAETAPGAS | GCAGTCAAGATGGCCATCAGTGGACTCTCTTTTTTCAGAATGG |
| | PGTSSTGSPGASPGTS | CAAAGTAAAGGTTTTTCAGGGAAATCAAGACTCCTTCACACCT |
| | STGSPGTPGSGTASSS | GTGGTGAACTCTCTAGACCCACCGTTACTGACTCGCTACCTTC |
| | PGSSTPSGATGSPGTP | GAATTCACCCCCAGAGTTGGGTGCACCAGATTGCCCTGAGGAT |
| | GSGTASSSPGSSTPSG | GGAGGTTCTGGGCTGCGAGGCACAGGACCTCTACGGCGCCGG |
| | ATGSPGTPGSGTASSS | ATCACCTGGGGCCGAAACGGCCCCGGGAGCGTCACCCGGTAC |
| | PGSSTPSGATGSPGSS | GTCCTCAACGGGGAGCCCTGGGGCATCACCCGGCACGTCCTCG |
| | TPSGATGSPGSSPSAS | ACAGGGTCCCCCGGAACACCGGGTTCGGGGACTGCGTCGTCGT |
| | TGTGPGSSPSASTGTG | CACCCGGTTCGTCAACACCCAGCGGTGCGACGGGTTCCCCGGG |
| | PGASPGTSSTGSPGTP | AACCCCCGGATCAGGAACAGCGTCGTCGAGCCCCGGAAGCTC |
| | GSGTASSSPGSSTPSG | GACCCCGTCAGGAGCGACCGGGTCGCCAGGAACGCCTGGGTC |
| | ATGSPGSSPSASTGTG | GGGTACTGCCTCATCGTCGCCGGGTTCATCGACGCCCTCCGGA |
| | PGSSPSASTGTGPGAS | GCAACAGGTTCACCCGGGTCGTCCACCCCCAGCGGAGCGACT |
| | PGTSSTGSPGASPGTS | GGATCACCGGGATCGAGCCCGTCGGCATCGACAGGAACAGGT |
| | STGSPGSSTPSGATGS | CCCGGTAGCTCCCCATCGGCCTCCACGGGGACGGGGCCTGGTG |
| | PGSSPSASTGTGPGAS | CGTCACCGGGGACAAGCAGCACGGGTTCGCCGGGAACGCCAG |
| | PGTSSTGSPGSSPSAS | GGTCAGGGACCGCGTCGTCGAGCCCAGGGTCGTCCACCCCAA |
| | TGTGPGTPGSGTASSS | GCGGGGCTACTGGGTCCCCGGGTAGCTCGCCGAGCGCATCAA |
| | PGSSTPSGATGSGAET | CGGGAACCGGACCGGGCAGCTCCCCCTCAGCGTCGACAGGGA |
| | AEQKLISEEDLSPATG | CAGGCCCTGGCGCGAGCCCGGGAACATCGTCGACGGGGTCAC |
| | | CCGGAGCATCGCCAGGGACCTCGTCAACTGGCTCGCCTGGATC |
| | | GTCAACGCCCTCGGGAGCCACGGGGTCGCCCGGATCATCCCCG |
| | | TCCGCCTCAACTGGCACAGGCCCTGGTGCTTCCCCTGGAACGT |
| | | CCAGCACAGGCTCCCCCGGTTCCAGCCCTTCCGCTTCGACTGG |
| | | GACTGGACCCGGAACCCCGGGATCGGGGACAGCGTCAAGCTC |
| | | GCCCGGAAGCTCCACGCCTTCGGGGGCAACCGGGTCAGGGGC |
| | | CGAAACGGCCGAACAAAAACTCATCTCAGAAGAGGATCTGTC |
| | | ACCTGCAACCGGTTGA |
| pBC0209 | MQIELSTCFFLCLLRF | ATGCAAATAGAGCTCTCCACCTGCTTCTTTCTGTGCCTTTTGCG |
| | CFSATRRYYLGAVEL | ATTCTGCTTTAGTGCCACCAGAAGATACTACCTGGGTGCAGTG |
| | SWDYMQSDLGELPV | GAACTGTCATGGGACTATATGCAAAGTGATCTCGGTGAGCTGC |
| | DARFPPRVPKSFPFNT | CTGTGGACGCAAGATTTCCTCCTAGAGTGCCAAAATCTTTTCC |
| | SVVYKKTLFVEFTDH | ATTCAACACCTCAGTCGTGTACAAAAAGACTCTGTTTGTAGAA |
| | LFNIAKPRPPWMGLL | TTCACGGATCACCTTTTCAACATCGCTAAGCCAAGGCCACCCT |
| | GPTIQAEVYDTVVITL | GGATGGGTCTGCTAGGTCCTACCATCCAGGCTGAGGTTTATGA |
| | KNMASHPVSLHAVG | TACAGTGGTCATTACACTTAAGAACATGGCTTCCCATCCTGTC |
| | VSYWKASEGAEYDD | AGTCTTCATGCTGTTGGTGTATCCTACTGGAAAGCTTCTGAGG |
| | QTSQREKEDDKVFPG | GAGCTGAATATGATGATCAGACCAGTCAAAGGGAGAAAGAAG |
| | GSHTYVWQVLKENG | ATGATAAAGTCTTCCCTGGTGGAAGCCATACATATGTCTGGCA |
| | PMASDPLCLTYSYLS | GGTCCTGAAAGAGAATGGTCCAATGGCCTCTGACCCACTGTGC |
| | HVDLVKDLNSGLIGA | CTTACCTACTCATATCTTTCTCATGTGGACCTGGTAAAAGACTT |
| | LLVCREGSLAKEKTQ | GAATTCAGGCCTCATTGGAGCCCTACTAGTATGTAGAGAAGGG |
| | TLHKFILLFAVFDEGK | AGTCTGGCCAAGGAAAAGACACAGACCTTGCACAAATTTATA |
| | SWHSETKNSLMQDR | CTACTTTTTGCTGTATTTGATGAAGGGAAAAGTTGGCACTCAG |
| | DAASARAWPKMHTV | AAACAAAGAACTCCTTGATGCAGGATAGGGATGCTGCATCTG |
| | NGYVNRSLPGLIGCH | CTCGGGCCTGGCCTAAAATGCACACAGTCAATGGTTATGTAAA |
| | RKSVYWHVIGMGTT | CAGGTCTCTGCCAGGTCTGATTGGATGCCACAGGAAATCAGTC |
| | PEVHSIFLEGHTFLVR | TATTGGCATGTGATTGGAATGGGCACCACTCCTGAAGTGCACT |
| | NHRQASLEISPITFLT | CAATATTCCTCGAAGGTCACACATTTCTTGTGAGGAACCATCG |
| | AQTLLMDLGQFLLFC | CCAGGCTAGCTTGGAAATCTCGCCAATAACTTTCCTTACTGCT |
| | HISSHQHDGMEAYVK | CAAACACTCTTGATGGACCTTGGACAGTTTCTACTGTTTTGTCA |
| | VDSCPEEPQLRMKNN | TATCTCTTCCCACCAACATGATGGCATGGAAGCTTATGTCAAA |
| | EEAEDYDDDLTDSEM | GTAGACAGCTGTCCAGAGGAACCCCAACTACGAATGAAAAAT |
| | DVVRFDDDNSPSFIQI | AATGAAGAAGCGGAAGACTATGATGATGATCTTACTGATTCTG |
| | RSVAKKHPKTWVHYI | AAATGGATGTGGTCAGGTTTGATGATGACAACTCTCCTTCCTT |
| | AAEEEDWDYAPLVL | TATCCAAATTCGCTCAGTTGCCAAGAAGCATCCTAAAACTTGG |
| | APDDRSYKSQYLNNG | GTACATTACATTGCTGCTGAAGAGGAGGACTGGGACTATGCTC |
| | PQRIGRKYKKVRFMA | CCTTAGTCCTCGCCCCCGATGACAGAAGTTATAAAAGTCAATA |
| | YTDETFKTREAIQHES | TTTGAACAATGGCCCTCAGCGGATTGGTAGGAAGTACAAAAA |
| | GILGPLLYGEVGDTL | AGTCCGATTTATGGCATACACAGATGAAACCTTTAAGACTCGT |
| | LIIFKNQASRPYNIYP | GAAGCTATTCAGCATGAATCAGGAATCTTGGGACCTTTACTTT |
| | HGITDVRPLYSRRLPK | ATGGGGAAGTTGGAGACACACTGTTGATTATATTTAAGAATCA |
| | GVKHLKDFPILPGEIF | AGCAAGCAGACCATATAACATCTACCCTCACGGAATCACTGAT |
| | KYKWTVTVEDGPTK | GTCCGTCCTTTGTATTCAAGGAGATTACCAAAAGGTGTAAAAC |
| | SDPRCLTRYYSSFVN | ATTTGAAGGATTTTCCAATTCTGCCAGGAGAAATATTCAAATA |
| | MERDLASGLIGPLLIC | TAAATGGACAGTGACTGTAGAAGATGGGCCAACTAAATCAGA |
| | YKESVDQRGNQIMSD | TCCTCGGTGCCTGACCCGCTATTACTCTAGTTTCGTTAATATGG |
| | KRNVILFSVFDENRS | AGAGAGATCTAGCTTCAGGACTCATTGGCCCTCTCCTCATCTG |
| | WYLTENIQRFLPNPA | CTACAAAGAATCTGTAGATCAAAGAGGAAACCAGATAATGTC |
| | GVQLEDPEFQASNIM | AGACAAGAGGAATGTCATCCTGTTTTCTGTATTTGATGAGAAC |
| | HSINGYVFDSLQLSV | CGAAGCTGGTACCTCACAGAGAATATACAACGCTTTCTCCCCA |
| | CLHEVAYWYILSIGA | ATCCAGCTGGAGTGCAGCTTGAGGATCCAGAGTTCCAAGCCTC |
| | QTDFLSVFFSGYTFK | CAACATCATGCACAGCATCAATGGCTATGTTTTTGATAGTTTG |
| | HKMVYEDTLTLFPFS | CAGTTGTCAGTTTGTTTGCATGAGGTGGCATACTGGTACATTCT |
| | GETVFMSMENPGLWI | AAGCATTGGAGCACAGACTGACTTCCTTTCTGTCTTCTTCTCTG |
| | LGCHNSDFRNRGMT | GATATACCTTCAAACACAAAATGGTCTATGAAGACACACTCAC |
| | ALLKVSSCDKNTGDY | CCTATTCCCATTCTCAGGAGAAACTGTCTTCATGTCGATGGAA |
| | YEDSYEDISAYLLSK | AACCCAGGTCTATGGATTCTGGGGTGCCACAACTCAGACTTTC |
| | NNAIEPRSFSQNGAPG | GGAACAGAGGCATGACCGCCTTACTGAAGGTTTCTAGTTGTGA |
| | TSESATPESGPGSEPA | CAAGAACACTGGTGATTATTACGAGGACAGTTATGAAGATATT |
| | TSGSETPGTSESATPE | TCAGCATACTTGCTGAGTAAAAACAATGCCATTGAACCAAGA |
| | SGPGSEPATSGSETPG | AGCTTCTCTCAAAACGGCGCGCCAGGTACCTCAGAGTCTGCTA |
| | TSESATPESGPGTSTE | CCCCCGAGTCAGGGCCAGGATCAGAGCCAGCCACCTCCGGGT |
| | PSEGSAPGSPAGSPTS | CTGAGACACCCGGGACTTCCGAGAGTGCCACCCCTGAGTCCGG |
| | TEEGTSESATPESGPG | ACCCGGGTCCGAGCCCGCCACTTCCGGCTCCGAAACTCCCGGC |
| | SEPATSGSETPGTSES | ACAAGCGAGAGCGCTACCCCAGAGTCAGGACCAGGAACATCT |
| | ATPESGPGSPAGSPTS | ACAGAGCCCTCTGAAGGCTCCGCTCCAGGGTCCCCAGCCGGCA |
| | TEEGSPAGSPTSTEEG | GTCCCACTAGCACCGAGGAGGGAACCTCTGAAAGCGCCACAC |
| | TSTEPSEGSAPGTSES | CCGAATCAGGGCCAGGGTCTGAGCCTGCTACCAGCGGCAGCG |
| | ATPESGPGTSESATPE | AGACACCAGGCACCTCTGAGTCCGCCACACCAGAGTCCGGAC |
| | SGPGTSESATPESGPG | CCGGATCTCCCGCTGGGAGCCCCACCTCCACTGAGGAGGGATC |
| | SEPATSGSETPGSEPA | TCCTGCTGGCTCTCCAACATCTACTGAGGAAGGTACCTCAACC |
| | TSGSETPGSPAGSPTS | GAGCCATCCGAGGGATCAGCTCCCGGCACCTCAGAGTCGGCA |
| | TEEGTSTEPSEGSAPG | ACCCCGGAGTCTGGACCCGGAACTTCCGAAAGTGCCACACCA |
| | TSTEPSEGSAPGSEPA | GAGTCCGGTCCCGGGACTTCAGAATCAGCAACACCCGAGTCC |
| | TSGSETPGTSESATPE | GGCCCTGGGTCTGAACCCGCCACAAGTGGTAGTGAGACACCA |
| | SGPGTSTEPSEGSAPA | GGATCAGAACCTGCTACCTCAGGGTCAGAGACACCCGGATCTC |
| | SSPPVLKRHQREITRT | CGGCAGGCTCACCAACCTCCACTGAGGAGGGCACCAGCACAG |
| | TLQSDQEEIDYDDTIS | AACCAAGCGAGGGCTCCGCACCCGGAACAAGCACTGAACCCA |
| | VEMKKEDFDIYDEDE | GTGAGGGTTCAGCACCCGGCTCTGAGCCGGCCACAAGTGGCA |
| | NQSPRSFQKKTRHYFI | GTGAGACACCCGGCACTTCAGAGAGTGCCACCCCCGAGAGTG |
| | AAVERLWDYGMSSS | GCCCAGGCACTAGTACCGAGCCCTCTGAAGGCAGTGCGCCAG |
| | PHVLRNRAQSGSVPQ | CCTCGAGCCCACCAGTCTTGAAACGCCATCAACGGGAAATAA |
| | FKKVVFQEFTDGSFT | CTCGTACTACTCTTCAGTCAGATCAAGAGGAAATCGATTATGA |
| | QPLYRGELNEHLGLL | TGATACCATATCAGTTGAAATGAAGAAGGAAGATTTTGACATT |
| | GPYIRAEVEDNIMVT | TATGATGAGGATGAAAATCAGAGCCCCCGCAGCTTTCAAAAG |
| | FRNQASRPYSFYSSLI | AAAACACGACACTATTTTATTGCTGCAGTGGAGAGGCTCTGGG |
| | SYEEDQRQGAEPRKN | ATTATGGGATGAGTAGCTCCCCACATGTTCTAAGAAACAGGGC |
| | FVKPNETKTYFWKV | TCAGAGTGGCAGTGTCCCTCAGTTCAAGAAAGTTGTTTTCCAG |
| | QHHMAPTKDEFDCK | GAATTTACTGATGGCTCCTTTACTCAGCCCTTATACCGTGGAG |
| | AWAYFSDVDLEKDV | AACTAAATGAACATTTGGGACTCCTGGGGCCATATATAAGAGC |
| | HSGLIGPLLVCHTNTL | AGAAGTTGAAGATAATATCATGGTAACTTTCAGAAATCAGGCC |
| | NPAHGRQVTVQEFAL | TCTCGTCCCTATTCCTTCTATTCTAGCCTTATTTCTTATGAGGA |
| | FFTIFDETKSWYFTEN | AGATCAGAGGCAAGGAGCAGAACCTAGAAAAAACTTTGTCAA |
| | MERNCRAPCNIQMED | GCCTAATGAAACCAAAACTTACTTTTGGAAAGTGCAACATCAT |
| | PTFKENYRFHAINGYI | ATGGCACCCACTAAAGATGAGTTTGACTGCAAAGCCTGGGCTT |
| | MDTLPGLVMAQDQR | ATTTCTCTGATGTTGACCTGGAAAAAGATGTGCACTCAGGCCT |
| | IRWYLLSMGSNENIH | GATTGGACCCCTTCTGGTCTGCCACACTAACACACTGAACCCT |
| | SIHFSGHVFTVRKKEE | GCTCATGGGAGACAAGTGACAGTACAGGAATTTGCTCTGTTTT |
| | YKMALYNLYPGVFE | TCACCATCTTTGATGAGACCAAAAGCTGGTACTTCACTGAAAA |
| | TVEMLPSKAGIWRVE | TATGGAAAGAAACTGCAGGGCTCCCTGCAATATCCAGATGGA |
| | CLIGEHLHAGMSTLF | AGATCCCACTTTTAAAGAGAATTATCGCTTCCATGCAATCAAT |
| | LVYSNKCQTPLGMAS | GGCTACATAATGGATACACTACCTGGCTTAGTAATGGCTCAGG |
| | GHIRDFQITASGQYG | ATCAAAGGATTCGATGGTATCTGCTCAGCATGGGCAGCAATGA |
| | QWAPKLARLHYSGSI | AAACATCCATTCTATTCATTTCAGTGGACATGTGTTCACTGTAC |
| | NAWSTKEPFSWIKVD | GAAAAAAAGAGGAGTATAAAATGGCACTGTACAATCTCTATC |
| | LLAPMIIHGIKTQGAR | CAGGTGTTTTTGAGACAGTGGAAATGTTACCATCCAAAGCTGG |
| | QKFSSLYISQFIIMYSL | AATTTGGCGGGTGGAATGCCTTATTGGCGAGCATCTACATGCT |
| | DGKKWQTYRGNSTG | GGGATGAGCACACTTTTTCTGGTGTACAGCAATAAGTGTCAGA |
| | TLMVFFGNVDSSGIK | CTCCCCTGGGAATGGCTTCTGGACACATTAGAGATTTTCAGAT |
| | HNIFNPPIIARYIRLHP | TACAGCTTCAGGACAATATGGACAGTGGGCCCCAAAGCTGGC |
| | THYSIRSTLRMELMG | CAGACTTCATTATTCCGGATCAATCAATGCCTGGAGCACCAAG |
| | CDLNSCSMPLGMESK | GAGCCCTTTTCTTGGATCAAGGTGGATCTGTTGGCACCAATGA |
| | AISDAQITASSYFTNM | TTATTCACGGCATCAAGACCCAGGGTGCCCGTCAGAAGTTCTC |
| | FATWSPSKARLHLQG | CAGCCTCTACATCTCTCAGTTTATCATCATGTATAGTCTTGATG |
| | RSNAWRPQVNNPKE | GGAAGAAGTGGCAGACTTATCGAGGAAATTCCACTGGAACCT |
| | WLQVDFQKTMKVTG | TAATGGTCTTCTTTGGCAATGTGGATTCATCTGGGATAAAACA |
| | VTTQGVKSLLTSMYV | CAATATTTTTAACCCTCCAATTATTGCTCGATACATCCGTTTGC |
| | KEFLISSSQDGHQWT | ACCCAACTCATTATAGCATTCGCAGCACTCTTCGCATGGAGTT |
| | LFFQNGKVKVFQGN | GATGGGCTGTGATTTAAATAGTTGCAGCATGCCATTGGGAATG |
| | QDSFTPVVNSLDPPLL | GAGAGTAAAGCAATATCAGATGCACAGATTACTGCTTCATCCT |
| | TRYLRIHPQSWVHQI | ACTTTACCAATATGTTTGCCACCTGGTCTCCTTCAAAAGCTCGA |
| | ALRMEVLGCEAQDL | CTTCACCTCCAAGGGAGGAGTAATGCCTGGAGACCTCAGGTG |
| | YGAGSPGAETAPGAS | AATAATCCAAAAGAGTGGCTGCAAGTGGACTTCCAGAAGACA |
| | PGTSSTGSPGASPGTS | ATGAAAGTCACAGGAGTAACTACTCAGGGAGTAAAATCTCTG |
| | STGSPGTPGSGTASSS | CTTACCAGCATGTATGTGAAGGAGTTCCTCATCTCCAGCAGTC |
| | PGSSTPSGATGSPGTP | AAGATGGCCATCAGTGGACTCTCTTTTTTCAGAATGGCAAAGT |
| | GSGTASSSPGSSTPSG | AAAGGTTTTTCAGGGAAATCAAGACTCCTTCACACCTGTGGTG |
| | ATGSPGTPGSGTASSS | AACTCTCTAGACCCACCGTTACTGACTCGCTACCTTCGAATTC |
| | PGSSTPSGATGSPGSS | ACCCCCAGAGTTGGGTGCACCAGATTGCCCTGAGGATGGAGG |
| | TPSGATGSPGSSPSAS | TTCTGGGCTGCGAGGCACAGGACCTCTACGGCGCCGGATCACC |
| | TGTGPGSSPSASTGTG | TGGGGCCGAAACGGCCCCGGGAGCGTCACCCGGTACGTCCTC |
| | PGASPGTSSTGSPGTP | AACGGGGAGCCCTGGGGCATCACCCGGCACGTCCTCGACAGG |
| | GSGTASSSPGSSTPSG | GTCCCCCGGAACACCGGGTTCGGGGACTGCGTCGTCGTCACCC |
| | ATGSPGSSPSASTGTG | GGTTCGTCAACACCCAGCGGTGCGACGGGTTCCCCGGGAACCC |
| | PGSSPSASTGTGPGAS | CCGGATCAGGAACAGCGTCGTCGAGCCCCGGAAGCTCGACCC |
| | PGTSSTGSPGASPGTS | CGTCAGGAGCGACCGGGTCGCCAGGAACGCCTGGGTCGGGTA |
| | STGSPGSSTPSGATGS | CTGCCTCATCGTCGCCGGGTTCATCGACGCCCTCCGGAGCAAC |
| | PGSSPSASTGTGPGAS | AGGTTCACCCGGGTCGTCCACCCCCAGCGGAGCGACTGGATCA |
| | PGTSSTGSPGSSPSAS | CCGGGATCGAGCCCGTCGGCATCGACAGGAACAGGTCCCGGT |
| | TGTGPGTPGSGTASSS | AGCTCCCCATCGGCCTCCACGGGGACGGGGCCTGGTGCGTCAC |
| | PGSSTPSGATGSGAET | CGGGGACAAGCAGCACGGGTTCGCCGGGAACGCCAGGGTCAG |
| | AEQKLISEEDLSPATG | GGACCGCGTCGTCGAGCCCAGGGTCGTCCACCCCAAGCGGGG |
| | | CTACTGGGTCCCCGGGTAGCTCGCCGAGCGCATCAACGGGAA |
| | | CCGGACCGGGCAGCTCCCCCTCAGCGTCGACAGGGACAGGCC |
| | | CTGGCGCGAGCCCGGGAACATCGTCGACGGGGTCACCCGGAG |
| | | CATCGCCAGGGACCTCGTCAACTGGCTCGCCTGGATCGTCAAC |
| | | GCCCTCGGGAGCCACGGGGTCGCCCGGATCATCCCCGTCCGCC |
| | | TCAACTGGCACAGGCCCTGGTGCTTCCCCTGGAACGTCCAGCA |
| | | CAGGCTCCCCCGGTTCCAGCCCTTCCGCTTCGACTGGGACTGG |
| | | ACCCGGAACCCCGGGATCGGGGACAGCGTCAAGCTCGCCCGG |
| | | AAGCTCCACGCCTTCGGGGGCAACCGGGTCAGGGGCCGAAAC |
| | | GGCCGAACAAAAACTCATCTCAGAAGAGGATCTGTCACCTGC |
| | | AACCGGTTGA |
| pBC0210 | MQIELSTCFFLCLLRF | ATGCAAATAGAGCTCTCCACCTGCTTCTTTCTGTGCCTTTTGCG |
| | CFSATRGAPGSPAGS | ATTCTGCTTTAGTGCCACCAGAGGCGCGCCAGGTTCTCCTGCT |
| | PTSTEEGTSESATPES | GGCTCCCCCACCTCAACAGAAGAGGGGACAAGCGAAAGCGCT |
| | GPGSEPATSGSETPAS | ACGCCTGAGAGTGGCCCTGGCTCTGAGCCAGCCACCTCCGGCT |
| | SATRRYYLGAVELSW | CTGAAACCCCTGCCTCGAGCGCTACAAGAAGATACTACCTGGG |
| | DYMQSDLGELPVDA | TGCAGTGGAACTGTCATGGGACTATATGCAAAGTGATCTCGGT |
| | RFPPRVPKSFPFNTSV | GAGCTGCCTGTGGACGCAAGATTTCCTCCTAGAGTGCCAAAAT |
| | VYKKTLFVEFTDHLF | CTTTTCCATTCAACACCTCAGTCGTGTACAAAAAGACTCTGTTT |
| | NIAKPRPPWMGLLGP | GTAGAATTCACGGATCACCTTTTCAACATCGCTAAGCCAAGGC |
| | TIQAEVYDTVVITLK | CACCCTGGATGGGTCTGCTAGGTCCTACCATCCAGGCTGAGGT |
| | NMASHPVSLHAVGV | TTATGATACAGTGGTCATTACACTTAAGAACATGGCTTCCCAT |
| | SYWKASEGAEYDDQ | CCTGTCAGTCTTCATGCTGTTGGTGTATCCTACTGGAAAGCTTC |
| | TSQREKEDDKVFPGG | TGAGGGAGCTGAATATGATGATCAGACCAGTCAAAGGGAGAA |
| | SHTYVWQVLKENGP | AGAAGATGATAAAGTCTTCCCTGGTGGAAGCCATACATATGTC |
| | MASDPLCLTYSYLSH | TGGCAGGTCCTGAAAGAGAATGGTCCAATGGCCTCTGACCCAC |
| | VDLVKDLNSGLIGAL | TGTGCCTTACCTACTCATATCTTTCTCATGTGGACCTGGTAAAA |
| | LVCREGSLAKEKTQT | GACTTGAATTCAGGCCTCATTGGAGCCCTACTAGTATGTAGAG |
| | LHKFILLFAVFDEGKS | AAGGGAGTCTGGCCAAGGAAAAGACACAGACCTTGCACAAAT |
| | WHSETKNSLMQDRD | TTATACTACTTTTTGCTGTATTTGATGAAGGGAAAAGTTGGCA |
| | AASARAWPKMHTVN | CTCAGAAACAAAGAACTCCTTGATGCAGGATAGGGATGCTGC |
| | GYVNRSLPGLIGCHR | ATCTGCTCGGGCCTGGCCTAAAATGCACACAGTCAATGGTTAT |
| | KSVYWHVIGMGTTPE | GTAAACAGGTCTCTGCCAGGTCTGATTGGATGCCACAGGAAAT |
| | VHSIFLEGHTFLVRNH | CAGTCTATTGGCATGTGATTGGAATGGGCACCACTCCTGAAGT |
| | RQASLEISPITFLTAQT | GCACTCAATATTCCTCGAAGGTCACACATTTCTTGTGAGGAAC |
| | LLMDLGQFLLFCHISS | CATCGCCAGGCTAGCTTGGAAATCTCGCCAATAACTTTCCTTA |
| | HQHDGMEAYVKVDS | CTGCTCAAACACTCTTGATGGACCTTGGACAGTTTCTACTGTTT |
| | CPEEPQLRMKNNEEA | TGTCATATCTCTTCCCACCAACATGATGGCATGGAAGCTTATG |
| | EDYDDDLTDSEMDV | TCAAAGTAGACAGCTGTCCAGAGGAACCCCAACTACGAATGA |
| | VRFDDDNSPSFIQIRS | AAAATAATGAAGAAGCGGAAGACTATGATGATGATCTTACTG |
| | VAKKHPKTWVHYIA | ATTCTGAAATGGATGTGGTCAGGTTTGATGATGACAACTCTCC |
| | AEEEDWDYAPLVLAP | TTCCTTTATCCAAATTCGCTCAGTTGCCAAGAAGCATCCTAAA |
| | DDRSYKSQYLNNGPQ | ACTTGGGTACATTACATTGCTGCTGAAGAGGAGGACTGGGACT |
| | RIGRKYKKVRFMAYT | ATGCTCCCTTAGTCCTCGCCCCCGATGACAGAAGTTATAAAAG |
| | DETFKTREAIQHESGI | TCAATATTTGAACAATGGCCCTCAGCGGATTGGTAGGAAGTAC |
| | LGPLLYGEVGDTLLII | AAAAAAGTCCGATTTATGGCATACACAGATGAAACCTTTAAG |
| | FKNQASRPYNIYPHGI | ACTCGTGAAGCTATTCAGCATGAATCAGGAATCTTGGGACCTT |
| | TDVRPLYSRRLPKGV | TACTTTATGGGGAAGTTGGAGACACACTGTTGATTATATTTAA |
| | KHLKDFPILPGEIFKY | GAATCAAGCAAGCAGACCATATAACATCTACCCTCACGGAAT |
| | KWTVTVEDGPTKSDP | CACTGATGTCCGTCCTTTGTATTCAAGGAGATTACCAAAAGGT |
| | RCLTRYYSSFVNMER | GTAAAACATTTGAAGGATTTTCCAATTCTGCCAGGAGAAATAT |
| | DLASGLIGPLLICYKE | TCAAATATAAATGGACAGTGACTGTAGAAGATGGGCCAACTA |
| | SVDQRGNQIMSDKRN | AATCAGATCCTCGGTGCCTGACCCGCTATTACTCTAGTTTCGTT |
| | VILFSVFDENRSWYL | AATATGGAGAGAGATCTAGCTTCAGGACTCATTGGCCCTCTCC |
| | TENIQRFLPNPAGVQL | TCATCTGCTACAAAGAATCTGTAGATCAAAGAGGAAACCAGA |
| | EDPEFQASNIMHSING | TAATGTCAGACAAGAGGAATGTCATCCTGTTTTCTGTATTTGA |
| | YVFDSLQLSVCLHEV | TGAGAACCGAAGCTGGTACCTCACAGAGAATATACAACGCTTT |
| | AYWYILSIGAQTDFLS | CTCCCCAATCCAGCTGGAGTGCAGCTTGAGGATCCAGAGTTCC |
| | VFFSGYTFKHKMVYE | AAGCCTCCAACATCATGCACAGCATCAATGGCTATGTTTTTGA |
| | DTLTLFPFSGETVFMS | TAGTTTGCAGTTGTCAGTTTGTTTGCATGAGGTGGCATACTGGT |
| | MENPGLWILGCHNSD | ACATTCTAAGCATTGGAGCACAGACTGACTTCCTTTCTGTCTTC |
| | FRNRGMTALLKVSSC | TTCTCTGGATATACCTTCAAACACAAAATGGTCTATGAAGACA |
| | DKNTGDYYEDSYEDI | CACTCACCCTATTCCCATTCTCAGGAGAAACTGTCTTCATGTCG |
| | SAYLLSKNNAIEPRSF | ATGGAAAACCCAGGTCTATGGATTCTGGGGTGCCACAACTCAG |
| | SQNGAPGTSESATPES | ACTTTCGGAACAGAGGCATGACCGCCTTACTGAAGGTTTCTAG |
| | GPGSEPATSGSETPGT | TTGTGACAAGAACACTGGTGATTATTACGAGGACAGTTATGAA |
| | SESATPESGPGSEPAT | GATATTTCAGCATACTTGCTGAGTAAAAACAATGCCATTGAAC |
| | SGSETPGTSESATPES | CAAGAAGCTTCTCTCAAAACGGCGCGCCAGGTACCTCAGAGTC |
| | GPGTSTEPSEGSAPGS | TGCTACCCCCGAGTCAGGGCCAGGATCAGAGCCAGCCACCTCC |
| | PAGSPTSTEEGTSESA | GGGTCTGAGACACCCGGGACTTCCGAGAGTGCCACCCCTGAGT |
| | TPESGPGSEPATSGSE | CCGGACCCGGGTCCGAGCCCGCCACTTCCGGCTCCGAAACTCC |
| | TPGTSESATPESGPGS | CGGCACAAGCGAGAGCGCTACCCCAGAGTCAGGACCAGGAAC |
| | PAGSPTSTEEGSPAGS | ATCTACAGAGCCCTCTGAAGGCTCCGCTCCAGGGTCCCCAGCC |
| | PTSTEEGTSTEPSEGS | GGCAGTCCCACTAGCACCGAGGAGGGAACCTCTGAAAGCGCC |
| | APGTSESATPESGPGT | ACACCCGAATCAGGGCCAGGGTCTGAGCCTGCTACCAGCGGC |
| | SESATPESGPGTSESA | AGCGAGACACCAGGCACCTCTGAGTCCGCCACACCAGAGTCC |
| | TPESGPGSEPATSGSE | GGACCCGGATCTCCCGCTGGGAGCCCCACCTCCACTGAGGAG |
| | TPGSEPATSGSETPGS | GGATCTCCTGCTGGCTCTCCAACATCTACTGAGGAAGGTACCT |
| | PAGSPTSTEEGTSTEP | CAACCGAGCCATCCGAGGGATCAGCTCCCGGCACCTCAGAGT |
| | SEGSAPGTSTEPSEGS | CGGCAACCCCGGAGTCTGGACCCGGAACTTCCGAAAGTGCCA |
| | APGSEPATSGSETPGT | CACCAGAGTCCGGTCCCGGGACTTCAGAATCAGCAACACCCG |
| | SESATPESGPGTSTEP | AGTCCGGCCCTGGGTCTGAACCCGCCACAAGTGGTAGTGAGA |
| | SEGSAPASSPPVLKRH | CACCAGGATCAGAACCTGCTACCTCAGGGTCAGAGACACCCG |
| | QREITRTTLQSDQEEI | GATCTCCGGCAGGCTCACCAACCTCCACTGAGGAGGGCACCA |
| | DYDDTISVEMKKEDF | GCACAGAACCAAGCGAGGGCTCCGCACCCGGAACAAGCACTG |
| | DIYDEDENQSPRSFQ | AACCCAGTGAGGGTTCAGCACCCGGCTCTGAGCCGGCCACAA |
| | KKTRHYFIAAVERLW | GTGGCAGTGAGACACCCGGCACTTCAGAGAGTGCCACCCCCG |
| | DYGMSSSPHVLRNRA | AGAGTGGCCCAGGCACTAGTACCGAGCCCTCTGAAGGCAGTG |
| | QSGSVPQFKKVVFQE | CGCCAGCCTCGAGCCCACCAGTCTTGAAACGCCATCAACGGG |
| | FTDGSFTQPLYRGEL | AAATAACTCGTACTACTCTTCAGTCAGATCAAGAGGAAATCGA |
| | NEHLGLLGPYIRAEV | TTATGATGATACCATATCAGTTGAAATGAAGAAGGAAGATTTT |
| | EDNIMVTFRNQASRP | GACATTTATGATGAGGATGAAAATCAGAGCCCCCGCAGCTTTC |
| | YSFYSSLISYEEDQRQ | AAAAGAAAACACGACACTATTTTATTGCTGCAGTGGAGAGGC |
| | GAEPRKNFVKPNETK | TCTGGGATTATGGGATGAGTAGCTCCCCACATGTTCTAAGAAA |
| | TYFWKVQHHMAPTK | CAGGGCTCAGAGTGGCAGTGTCCCTCAGTTCAAGAAAGTTGTT |
| | DEFDCKAWAYFSDV | TTCCAGGAATTTACTGATGGCTCCTTTACTCAGCCCTTATACCG |
| | DLEKDVHSGLIGPLL | TGGAGAACTAAATGAACATTTGGGACTCCTGGGGCCATATATA |
| | VCHTNTLNPAHGRQ | AGAGCAGAAGTTGAAGATAATATCATGGTAACTTTCAGAAAT |
| | VTVQEFALFFTIFDET | CAGGCCTCTCGTCCCTATTCCTTCTATTCTAGCCTTATTTCTTAT |
| | KSWYFTENMERNCR | GAGGAAGATCAGAGGCAAGGAGCAGAACCTAGAAAAAACTTT |
| | APCNIQMEDPTFKEN | GTCAAGCCTAATGAAACCAAAACTTACTTTTGGAAAGTGCAAC |
| | YRFHAINGYIMDTLP | ATCATATGGCACCCACTAAAGATGAGTTTGACTGCAAAGCCTG |
| | GLVMAQDQRIRWYL | GGCTTATTTCTCTGATGTTGACCTGGAAAAAGATGTGCACTCA |
| | LSMGSNENIHSIHFSG | GGCCTGATTGGACCCCTTCTGGTCTGCCACACTAACACACTGA |
| | HVFTVRKKEEYKMA | ACCCTGCTCATGGGAGACAAGTGACAGTACAGGAATTTGCTCT |
| | LYNLYPGVFETVEML | GTTTTTCACCATCTTTGATGAGACCAAAAGCTGGTACTTCACT |
| | PSKAGIWRVECLIGE | GAAAATATGGAAAGAAACTGCAGGGCTCCCTGCAATATCCAG |
| | HLHAGMSTLFLVYSN | ATGGAAGATCCCACTTTTAAAGAGAATTATCGCTTCCATGCAA |
| | KCQTPLGMASGHIRD | TCAATGGCTACATAATGGATACACTACCTGGCTTAGTAATGGC |
| | FQITASGQYGQWAPK | TCAGGATCAAAGGATTCGATGGTATCTGCTCAGCATGGGCAGC |
| | LARLHYSGSINAWST | AATGAAAACATCCATTCTATTCATTTCAGTGGACATGTGTTCA |
| | KEPFSWIKVDLLAPMI | CTGTACGAAAAAAAGAGGAGTATAAAATGGCACTGTACAATC |
| | IHGIKTQGARQKFSSL | TCTATCCAGGTGTTTTTGAGACAGTGGAAATGTTACCATCCAA |
| | YISQFIIMYSLDGKKW | AGCTGGAATTTGGCGGGTGGAATGCCTTATTGGCGAGCATCTA |
| | QTYRGNSTGTLMVFF | CATGCTGGGATGAGCACACTTTTTCTGGTGTACAGCAATAAGT |
| | GNVDSSGIKHNIFNPP | GTCAGACTCCCCTGGGAATGGCTTCTGGACACATTAGAGATTT |
| | IIARYIRLHPTHYSIRS | TCAGATTACAGCTTCAGGACAATATGGACAGTGGGCCCCAAA |
| | TLRMELMGCDLNSCS | GCTGGCCAGACTTCATTATTCCGGATCAATCAATGCCTGGAGC |
| | MPLGMESKAISDAQI | ACCAAGGAGCCCTTTTCTTGGATCAAGGTGGATCTGTTGGCAC |
| | TASSYFTNMFATWSP | CAATGATTATTCACGGCATCAAGACCCAGGGTGCCCGTCAGAA |
| | SKARLHLQGRSNAW | GTTCTCCAGCCTCTACATCTCTCAGTTTATCATCATGTATAGTC |
| | RPQVNNPKEWLQVD | TTGATGGGAAGAAGTGGCAGACTTATCGAGGAAATTCCACTG |
| | FQKTMKVTGVTTQG | GAACCTTAATGGTCTTCTTTGGCAATGTGGATTCATCTGGGAT |
| | VKSLLTSMYVKEFLIS | AAAACACAATATTTTTAACCCTCCAATTATTGCTCGATACATC |
| | SSQDGHQWTLFFQNG | CGTTTGCACCCAACTCATTATAGCATTCGCAGCACTCTTCGCAT |
| | KVKVFQGNQDSFTPV | GGAGTTGATGGGCTGTGATTTAAATAGTTGCAGCATGCCATTG |
| | VNSLDPPLLTRYLRIH | GGAATGGAGAGTAAAGCAATATCAGATGCACAGATTACTGCT |
| | PQSWVHQIALRMEVL | TCATCCTACTTTACCAATATGTTTGCCACCTGGTCTCCTTCAAA |
| | GCEAQDLYGAGSPG | AGCTCGACTTCACCTCCAAGGGAGGAGTAATGCCTGGAGACCT |
| | AETAPGASPGTSSTGS | CAGGTGAATAATCCAAAAGAGTGGCTGCAAGTGGACTTCCAG |
| | PGASPGTSSTGSPGTP | AAGACAATGAAAGTCACAGGAGTAACTACTCAGGGAGTAAAA |
| | GSGTASSSPGSSTPSG | TCTCTGCTTACCAGCATGTATGTGAAGGAGTTCCTCATCTCCA |
| | ATGSPGTPGSGTASSS | GCAGTCAAGATGGCCATCAGTGGACTCTCTTTTTTCAGAATGG |
| | PGSSTPSGATGSPGTP | CAAAGTAAAGGTTTTTCAGGGAAATCAAGACTCCTTCACACCT |
| | GSGTASSSPGSSTPSG | GTGGTGAACTCTCTAGACCCACCGTTACTGACTCGCTACCTTC |
| | ATGSPGSSTPSGATGS | GAATTCACCCCCAGAGTTGGGTGCACCAGATTGCCCTGAGGAT |
| | PGSSPSASTGTGPGSS | GGAGGTTCTGGGCTGCGAGGCACAGGACCTCTACGGCGCCGG |
| | PSASTGTGPGASPGTS | ATCACCTGGGGCCGAAACGGCCCCGGGAGCGTCACCCGGTAC |
| | STGSPGTPGSGTASSS | GTCCTCAACGGGGAGCCCTGGGGCATCACCCGGCACGTCCTCG |
| | PGSSTPSGATGSPGSS | ACAGGGTCCCCCGGAACACCGGGTTCGGGGACTGCGTCGTCGT |
| | PSASTGTGPGSSPSAS | CACCCGGTTCGTCAACACCCAGCGGTGCGACGGGTTCCCCGGG |
| | TGTGPGASPGTSSTGS | AACCCCCGGATCAGGAACAGCGTCGTCGAGCCCCGGAAGCTC |
| | PGASPGTSSTGSPGSS | GACCCCGTCAGGAGCGACCGGGTCGCCAGGAACGCCTGGGTC |
| | TPSGATGSPGSSPSAS | GGGTACTGCCTCATCGTCGCCGGGTTCATCGACGCCCTCCGGA |
| | TGTGPGASPGTSSTGS | GCAACAGGTTCACCCGGGTCGTCCACCCCCAGCGGAGCGACT |
| | PGSSPSASTGTGPGTP | GGATCACCGGGATCGAGCCCGTCGGCATCGACAGGAACAGGT |
| | GSGTASSSPGSSTPSG | CCCGGTAGCTCCCCATCGGCCTCCACGGGGACGGGGCCTGGTG |
| | ATGSGAETAEQKLISE | CGTCACCGGGGACAAGCAGCACGGGTTCGCCGGGAACGCCAG |
| | EDLSPATG | GGTCAGGGACCGCGTCGTCGAGCCCAGGGTCGTCCACCCCAA |
| | | GCGGGGCTACTGGGTCCCCGGGTAGCTCGCCGAGCGCATCAA |
| | | CGGGAACCGGACCGGGCAGCTCCCCCTCAGCGTCGACAGGGA |
| | | CAGGCCCTGGCGCGAGCCCGGGAACATCGTCGACGGGGTCAC |
| | | CCGGAGCATCGCCAGGGACCTCGTCAACTGGCTCGCCTGGATC |
| | | GTCAACGCCCTCGGGAGCCACGGGGTCGCCCGGATCATCCCCG |
| | | TCCGCCTCAACTGGCACAGGCCCTGGTGCTTCCCCTGGAACGT |
| | | CCAGCACAGGCTCCCCCGGTTCCAGCCCTTCCGCTTCGACTGG |
| | | GACTGGACCCGGAACCCCGGGATCGGGGACAGCGTCAAGCTC |
| | | GCCCGGAAGCTCCACGCCTTCGGGGGCAACCGGGTCAGGGGC |
| | | CGAAACGGCCGAACAAAAACTCATCTCAGAAGAGGATCTGTC |
| | | ACCTGCAACCGGTTGA |

### Example 21

### Transfection of Mammalian Cells, Expression of FVIII-XTEN and Assessment of FVIII Activity

Mammalian cells, including but not limited to CHO, BHK, COS, and HEK293, are suitable for transformation with the vectors of the Examples, above, in order to express and recover FVIII-XTEN fusion protein. The following are details for methods used to express BDD FVIII and FVIII-XTEN fusion protein constructs pBC0114, pBC0135, pBC0136, pBC0137, pBC0145, pBC0146, and pBC0149 bp transient transfection, which includes electroporation and chemical (PEI) transfection methods.

Adherent HEK293 cells purchased from ATCC were revived in medium of vendor's recommendation and passaged for a few generations before multiple vials were frozen in the medium with 5% DMSO. One vial was revived and passaged one more time before transfection. The HEK293 cells were plated 1-2 days before transfection at a density of approximately 7×10⁵ per ml in one T175 per transfection, using 35 ml medium. On the day of transfection the cells were trypsinized, detached and counted, then rinsed in the medium until an even cell suspension was achieved. The cells were counted and an appropriate volume of cells (based on cell count above) were transferred to 50 mL centrifuge tube, such that there were approximately 4×10⁶ cells per transfection. Cells were centrifuged for 5 min at 500 RCF, the supernatant discarded, and the cells resuspended in 10 ml of D-PBS.

Electroporation:

For electroporation, an appropriate volume of resuspension buffer was added using a micropipette (supplied in the Neon™ Transfection System 100 μL Kit), such that 110 μl of buffer was available per transfection. Separate volumes of 110 μl of cell suspension were added to each Eppendorf tube containing 11 μl of plasmid DNA for each of the individual FVIII-XTEN constructs for a total of 6 μg (volume of DNA may be less, qs to 11 ul with sterile H2O). A Neon™ Transfection Device was used for transfection. The program was set to electroporate at 1100 v for a pulse width of 20 ms, for a total of two pulses. A Neon™ Tube (supplied in the Neon™ Transfection System 100 μL Kit) was placed into Neon™ Pipette Station. A volume of 3 mL of Electrolytic Buffer E2 (supplied in the Neon™ Transfection System 100 μL Kit) was added to the Neon™ Tube. Neon™ Pipettes and 100 μl Neon™ Tips were used to electroporate 100 μl of cell-plasmid DNA mixture using the Neon™ Pipette Station. The electroporation was executed and when complete, the Neon™ Pipette was removed from the Station and the pipette with the transfected cells was used to transfer the cells, with a circular motion, into a 100 mm×20 mm petri plate containing 10 ml of Opti-MEM I Reduced-Serum Medium (1×, Invitrogen), such that transfected cells were evenly distributed on plate. The cells for each transfection were incubated at 37° C. for expression. On day 3 post-transfection, a 10% volume of salt solution of 10 mM Hepes, 5 mM CaCl₂, and 4M NaCl was added to each cell culture and gently mixed for 30 minutes. Each cell culture was transferred to a 50 ml conical centrifuge tube and was centrifuged at 3000 rpm for 10 minutes at 4° C. The supernatants for each culture were placed into a new 50 ml conical tube and then split into aliquots of 5×1 ml in Eppendorf and 2×15 ml conical tubes for assay or were flash frozen before testing for expression of FVIII-XTEN in ELISA and performance in an FVIII activity assay, as described herein.

Chemical Transfection:

Chemical transfection can be accomplished using standard methods known in the art. In the present Example, PEI is utilized, as described.

Suspension 293 Cells are seeded the day before transfection at 7×10⁵ cells/mL in sufficient Freestyle 293 (Invitrogen) medium to provide at least 30 ml working volume, and incubated at 37° C. On the day of transfection, an aliquot of 1.5 ml of the transfection medium is held at room temperature, to which 90 μL of 1 mg/ml PEI is added and vortexed briefly. A volume of 30 μl of DNA encoding the FVIII-XTEN_AE288 construct (concentration of 1 mg/ml) is added to the PEI solution, which is vortexed for 30 sec. The mixture is held at room temperature for 5-15 min. The DNA/PEI mixture is added to the HEK293 cells and the suspension is incubated at 37° C. using pre-established shake flask conditions. About four hours after the addition of the DNA/PEI mix, a 1× volume of expansion media is added and the cells incubated at 37° C. for 5 days. On the day of harvest, a 10% volume of salt solution of 10 mM Hepes, 5 mM CaCl₂, and 4M NaCl is added to the cell culture and gently mixed for 30 minutes. The cell culture is transferred to a 50 ml conical centrifuge tube and is centrifuged at 4000 rpm for 10 minutes at 4° C. The supernatant is placed into a new 50 ml conical tube and then split into aliquots of 5×1 ml in Eppendorf and 2×15 ml conical tubes for assay or are flash frozen before testing for expression of FVIII-XTEN in ELISA and/or performance in an FVIII activity assay, as described herein.

Assay of Expressed FVIII by ELISA

To verify and quantitate the expression of FVIII-XTEN fusion proteins of the constructs by cell culture, an ELISA assay was established. Capture antibodies, either SAF8C-AP (Affinity Biologicals), or GMA-8002 (Green Mountain Antibodies) were immobilized onto wells of an ELISA plate. The wells were then incubated with blocking buffer (1×PBS/3% BSA) to prevent non-specific binding of other proteins to the anti-FVIII antibody. FVIII standard dilutions (˜50 ng-0.024 ng range), quality controls, and cell culture media samples were then incubated for 1.5 h in the wells to allow binding of the expressed FVIII protein to the coated antibody. Wells were then washed extensively, and bound protein is incubated with anti-FVIII detection antibody, SAF8C-Biotinylated (Affinity Biologicals). Then streptavidin-HRP, which binds the biotin conjugated to the FVIII detection antibody, is added to the well and incubated for 1 h. Finally, OPD substrate is added to the wells and its hydrolysis by HRP enzyme is monitored with a plate reader at 490 nm wavelength. Concentrations of FVIII-containing samples were then calculated by comparing the colorimetric response at each culture dilution to a standard curve. The results, in Table 15, below, show that FVIII-XTEN of the various constructs are expressed at 0.4-1 μg/ml in the cell culture media. The results obtained by ELISA and the activity data indicate that FVIII-XTEN fusion proteins were very well expressed using the described transfection methods. Furthermore, under the experimental conditions, the results demonstrate that the specific activity values of the FVIII-XTEN proteins were similar or greater than that of pBC0114 base construct (expressing BDD FVIII) and support that XTEN insertion into the C-terminus or B-domain of FVIII results in preservation of FVIII protein function.

Activity Assay for CFXTEN Fusion Protein of FVIII BDD Linked to XTEN

BDD FVIII and FVIII-XTEN fusion protein constructs pBC0114, pBC0135, pBC0136, pBC0137, pBC0145, pBC0146, and pBC0149, in various configurations, including XTEN AE288 and AG288 inserted at the C-terminus of the FVIII BDD sequence and FVIII-XTEN fusion proteins with AE42 and AE288 inserted after residue 745 (or residue 743) and before residue 1640 (or residue 1638) of the B-domain (including constructs with the P1648 processing site mutated to alanine), were expressed in transiently transfected Freestyle 293 cells, as described above, and tested for procoagulant activity. The procoagulant activity of each of the FVIII-XTEN proteins present in cell culture medium was assessed using a Chromogenix Coamatic® Factor VIII assay, an assay in which the activation of factor X was linearly related to the amount of factor VIII in the sample. The assay was performed according to manufacturer's instructions using the end-point method, which was measured spectrophotometrically at OD405 nm. A standard curve was created using purified FVIII protein at concentrations of 250, 200, 150, 100, 75, 50, 37.5, 25, 12.5, 6.25, 3.125 and 1.56 mU/ml. Dilutions of factor VIII standard, quality controls, and samples were prepared with assay buffer and PEI culture medium to account for the effect of the medium in the assay performance. Positive controls consist of purified factor VIII protein at 20, 40, and 80 mU/ml concentrations and cell culture medium of pBC0114 FVIII base construct, lacking the XTEN insertions. Negative controls consisted of assay buffer or PEI culture medium alone. The cell culture media of the FVIII-XTEN constructs were obtained as described, above, and were tested in replicates at 1:50, 1:150, and 1:450 dilutions and the activity of each was calculated in U/ml. Each FVIII-XTEN construct exhibited procoagulant activity that was at least comparable, and in some cases greater than that of the base construct positive control, and support that under the conditions of the experiments, the linkage of XTEN, including AE288 or AG288, at the C-terminus of FVIII or insertion of XTEN, including AE42 or AE288 within the B-domain resulted in retention or even enhancement of FVIII procoagulant activity.

TABLE 15
Results of ELISA and Chromogenix FVIII activity assays

| FVIII- | | | | |
|---|---|---|---|---|
| XTEN | Activity | Concentration | Specific Activity | |
| Construct | (IU/ml) | (μg/ml) | (IU/mg) | Description of Construct |
| pBC0114 | 3.0 | 0.6 | 5000 | BDD FVIII base construct used for |
| | | | | XTEN insertions |
| pBC0146 | 7.4 | 0.6 | 12759 | FVIII construct with XTEN AG288 |
| | | | | inserted at the C-terminus of FVIII |
| pBC0145 | 3.1 | 0.6 | 4844 | FVIII construct with XTEN AE288 |
| | | | | inserted at the C-terminus of FVIII |
| pBC0135 | 4.0 | 1.0 | 4124 | FVIII construct with XTEN AE42 |
| | | | | inserted between residue 745 and 1640 |
| pBC0149 | 4.9 | 0.9 | 5581 | FVIII construct with XTEN AE42 |
| | | | | inserted between residue 745 and 1640 |
| | | | | and with Arg1648 to Ala mutation |
| pBC0136 | 2.7 | 0.4 | 7670 | FVIII construct with XTEN AE288 |
| | | | | inserted between residue 745 and 1640 |
| pBC0137 | 1.9 | 0.3 | 6013 | FVIII construct with XTEN AE288 |
| | | | | inserted between residue 745 and 1640 |
| | | | | and with Arg1648 to Ala mutation |

Generation of Stable Pools and Cell Lines that Produce FVIII-XTEN

Stable pools are generated by culturing transfected cells for 3-5 weeks in medium containing selection antibiotics such as puromycin, with medium change every 2-3 days. Stable cells can be used for either production or generation of stable clones. For stable cell line selection during primary screening, cells from stable pools either from on-going passaging or revived from frozen vials are seeded in 96-well plates at a target density of 0.5 cell/well. About 1 week after seeding spent medium from wells with single cell cluster as observed under microscope are tested for expression of FVIII by activity assay or antigen measurement.

For additional rounds of screening, normalized numbers of cells are seeded in multi-well plates. Spent medium is harvested and tested for FVIII concentration by ELISA and FVIII activity assay. Cells would also be harvested from the plates and counted using Vi-Cell. Clones are ranked by (1) FVIII titers according to ELISA and activity; (2) ratios of ELISA titer/cell count and activity titer/cell count; and (3) integrity and homogeneity of products produced by the clones as measured by Western blots. A number of clones for each of the constructs are selected from the primary screening for additional rounds of screening.

For the second round of screening, cells in 96-well plates for the top clones selected from primary screening are first expanded in T25 flasks and then seeded in duplicate 24-well plates. Spent medium is collected from the plates for FVIII activity and antigen quantification and cells harvested and counted by Vi-Cell. Clones are ranked and then selected according to titers by ELISA and activity assay, ELISA titer/cell and activity titer/cell count ratios. Frozen vials are prepared for at least 5-10 clones and again these clones were screened and ranked according to titers by ELISA and activity, and ratios of ELISA titer/cell count and activity titer/cell count, and product integrity and homogeneity by Western blot, and 2-3 clones are selected for productivity evaluation in shake flasks. Final clones are selected based on specific productivity and product quality.

Production of FVIII-XTEN Secreted in Cell Culture Medium by Suspension 293 Stable Clones

HEK293 stable cell clones selected by the foregoing methods are seeded in shake flasks at 1-2×10⁵ cells/ml in expression medium. Cell count, cell viability, FVIII activity and antigen expression titers are monitored daily. On the day when FVIII activity and antigen titers and product quality are optimal, the culture is harvested by either centrifugation/sterile filtration or depth filtration/sterile filtration. The filtrate is either used immediately for tangential flow filtration (TFF) processing and purification or stored in −80° C. freezer for TFF processing and purification later.

### Example 22

### Purification and Characterization of CFXTEN Constructs

Purification of FVII-XTEN_AE864 by FVIII Affinity Chromatography

CFXTEN containing supernatant is filtered using a Cuno ZetaPlus Biocap filter and a Cuno BioAssure capsule and subsequently concentrated by tangential flow filtration using a Millipore Pellicon 2 Mini cartridge with a 30,000 Da MWCO. Using the same tangential flow filtration cartridge the sample is diafiltered into 10 mM histidine, 20 mM calcium chloride, 300 mM sodium chloride, and 0.02% Tween 80 at pH 7.0. FVIIISelect resin (GE 17-5450-01) selectively binds FVIII or B domain deleted FVIII using a 13 kDa recombinant protein ligand coupled to a chromatography resin. The resin is equilibrated with 10 mM histidine, 20 mM calcium chloride, 300 mM sodium chloride, and 0.02% Tween 80 at pH 7.0 and the supernatant loaded. The column is washed with 20 mM histidine, 20 mM calcium chloride, 300 mM sodium chloride, and 0.02% Tween 80 at pH 6.5, then is washed with 20 mM histidine, 20 mM calcium chloride, 1.0 M sodium chloride, and 0.02% Tween 80 at pH 6.5, and eluted with 20 mM histidine, 20 mM calcium chloride, 1.5 M sodium chloride, and 0.02% Tween 80 dissolved in 50% ethylene glycol at pH 6.5.

Concentration and Buffer Exchange by Tangential Flow Filtration and Diafiltration

Supernatant batches totaling at least 10 L in volume, from stable CHO cells lines expressing CFXTEN are filtered using a Cuno ZetaPlus Biocap filter and a Cuno BioAssure capsule. They are subsequently concentrated approximately 20-fold by tangential flow filtration using a Millipore Pellicon 2 Mini cartridge with a 30,000 Da MWCO. Using the same tangential flow filtration cartridge the sample is diafiltered with 10 mM histidine, 20 mM calcium chloride, 300 mM sodium chloride, and 0.02% Tween 80 at pH 7.0 10 mM tris pH 7.5, 1 mM EDTA with 5 volumes worth of buffer exchange. Samples are divided into 50 ml aliquots and frozen at −80° C.

Purification of CFXTEN by Anion Exchange Chromatography

Using an Akta FPLC system the sample is purified using a SuperQ-650M column. The column is equilibrated into buffer A (0.02 mol/L imidazole, 0.02 mol/L glycine ethylester hydrochloride, 0.1 5 mol/L, NaCl, 2.5% glycerol, pH 6.9) and the sample loaded. The sample is eluted using buffer B (5 mmol/L histidine HCl (His/HCl), 1.15 mol/L NaCl, pH 6.0). The 215 nm chromatogram is used to monitor the elution profile. The eluted fractions are assayed for FVIII by ELISA, SDS-PAGE or activity assay. Peak fractions are pooled and stored or subjected to thrombin activation immediately (O'Brien et al., Blood (1990) 75:1664-1672). Fractions are assayed for FVIII activity using an aPTT based factor assay. A Bradford assay is performed to determine the total amount of protein in the load and elution fractions.

Purification of CFXTEN by Hydrophobic Interaction Chromatography

CFXTEN samples in Buffer A (50 mmol/l histidine, 1 mmol/l CaCl2, 1 M NaCl, and 0.2 g/l Tween 80®, pH 6.8) are loaded onto a toyopearl ether 650M resin equilibrated in Buffer A. The column is washed with 10 column volumes of Buffer A to remove DNA, incorrectly folded forms and FVIII, and other contaminant proteins. The CFXTEN is eluted with Buffer B (25 mmol/1 histidine, 0.5 mmol/l CaCl2 and 0.4 mol/l NaCl, pH 6.8) as a single step elution (U.S. Pat. No. 6,005,082). Fractions are assayed for FVIII activity using an aPTT based factor assay. A Bradford assay is performed to determine the total amount of protein in the load and elution fractions.

Removal of Aggregated Protein from Monomeric CFXTEN with Anion Exchange Chromatography

Using an Akta FPLC system the sample is purified using a macrocap Q column. The column is equilibrated into buffer A (20 mM MES, 1 mM CaCl2, pH 6.0) and the sample is loaded. The sample is eluted using a linear gradient of 30% to 80% buffer B (20 mM MES, 1 mM CaCl2, pH 6.0+500 mM NaCl) over 20 column volumes. The 215 nm chromatogram is used to monitor the elution profile. The fractions corresponding to the early portion of the elution contain primarily monomeric protein, while the late portion of the elution contains primarily the aggregated species. Fractions from the macrocapQ column is analyzed via size exclusion chromatography with 60 cm BioSep G4000 column to determine which to pool to create an aggregate free sample.

Activation of FVIII by Thrombin

Purified FVIII in 5 mmol/L histidine HCl (His/HCl), 1.15 mol/L NaCl, pH 6.0 is treated with thrombin at a 1:4 ratio of units of human thrombin to units FVIII, and the sample is incubated at 37° C. for up to 2 hours. To monitor the activation process, aliquots of this sample are then withdrawn, and acetone precipitated by the addition of 4.5 vol ice-cold acetone. The sample is incubated on ice for 10 minutes, and the precipitate is collected by centrifugation at 13,000 g in a microfuge for 3 minutes. The acetone is removed, and the precipitate is resuspended in 30 μL SDS-PAGE reducing sample buffer and boiled for 2 minutes. Samples are then assayed by SDS-PAGE or western blot. The conversion of FVIII to FVIIIa is examined by looking for the conversion of the heavy chain into 40 and 50 kDa fragments and the conversion of the light chain into a 70 kDa fragment (O'Brien et al., Blood (1990) 75:1664-1672).

SEC Analysis of CFXTEN

FVII-XTEN purified by affinity and anion exchange chromatography is analyzed by size exclusion chromatography with 60 cm BioSep G4000 column. A monodispersed population with a hydrodynamic radius of −10 nm/apparent MW of ˜1.7 MDa (XTEN-288 fusion) or ˜12 nm/an apparent MW of 5.3 MDa (XTEN-864 fusion) is indicative of an aggregation-free sample. CFXTEN is expected to have an apparent molecular weight factor up to or about 8 (for an XTEN-288 fusion with FVIII) or up to or about ˜15 (for an XTEN-864 fusion with FVIII).

ELISA Based Concentration Determination of CFXTEN

The quantitative determination of factor VIII/CFXTEN antigen concentrations using the double antibody enzyme linked immuno-sorbent assay (ELISA) is performed using proven antibody pairings (VisuLize™ FVIII Antigen kit, Affinity Biologicals, Ontario Canada). Strip wells are pre-coated with sheep polyclonal antibody to human FVIII. Plasma samples are diluted and applied to the wells. The FVIII antigen that is present binds to the coated antibody. After washing away unbound material, peroxidase-labeled sheep detecting antibody is applied and allowed to bind to the captured FVIII. The wells are again washed and a solution of TMB (the peroxidase substrate tetramethylbenzidine) is applied and allowed to react for a fixed period of time. A blue color develops which changes to yellow upon quenching the reaction with acid. The color formed is measured spectrophotometrically in a microplate reader at 450 nm. The absorbance at 450 nm is directly proportional to the quantity of FVIII antigen captured onto the well. The assay is calibrated using either the calibrator plasma provided in the kit or by substituting a CFXTEN standard in an appropriate matrix.

Assessment of CFXTEN Activity via a FXa Coupled Chromogenic Substrate Assay

Using the Chromogenix Coamatic Factor VIII (Chromogenix, cat #82258563) the activity of FVIII is assessed as follows. In the presence of calcium ions and phospholipids, factor X is activated to factor Xa by factor IXa. This activation is greatly stimulated by factor VIII which acts as a cofactor in this reaction. By using optimal amounts of Ca²⁺, phospholipid and factor IXa, and an excess of factor X, the rate of activation of factor X is linearly related to the amount of factor VIII. Factor Xa hydrolyses the chromogenic substrate S-2765 thus liberating the chromophoric group, pNA. The color is then read spectrophotometrically at 405 nm. The generated factor Xa and thus the intensity of color is proportional to the factor VIII activity in the sample. Hydrolysis of S-2765 by thrombin formed is prevented by the addition of the synthetic thrombin inhibitor I-2581 together with the substrate. The activity of an unknown sample is determined by comparing final A405 of that sample to those from a standard curve constructed from known FVIII amounts. By also determining the amount of FVIII antigen present in the samples (via A280 or ELISA), a specific activity of a sample is determine to understand the relative potency of a particular preparation of FVIII. This enables the relative efficiency of different isolation strategies or construct designs for CFXTEN fusions to be assessed for activity and ranked.

aPTT Based Assays for CFXTEN Activity Determination

CFXTEN acts to replace FVIII in the intrinsic or contact activated coagulation pathway. The activity of this coagulation pathway is assessed using an activated partial thromboplastin time assay (aPTT). FVIII activity specifically is measured as follows: a standard curve is prepared by diluting normal control plasma (Pacific Hemostasis cat #100595) two-fold with FVIII deficient plasma (cat #100800) and then conducting 6, 4-fold serial dilutions again with factor VIII deficient plasma. This creates a standard curve with points at 500, 130, 31, 7.8, 2.0, 0.5 and 0.1 IU/ml of activity, where one unit of activity is defined as the amount of FVIIIC activity in 1 ml of normal human plasma. A FVIII-deficient plasma also is included to determine the background level of activity in the null plasma. The sample is prepared by adding CFXTEN to FVIII deficient plasma at a ratio of 1:10 by volume. The samples is tested using an aPTT assay as follows. The samples are incubated at 37 C in a molecular devices plate reader spectrophotometer for 2 minutes at which point an equal volume of aPTT reagent (Pacific Hemostasis cat #100402) is added and an additional 3 minute 37 C incubation performed. After the incubation the assay is activated by adding one volume of calcium chloride (Pacific Hemostasis cat #100304). The turbidity is monitored at 450 nm for 5 minutes to create reaction profiles. The aPTT time, or time to onset of clotting activity, is defined as the first time where OD405 nm increased by 0.06 over baseline. A log-linear standard curve is created with the log of activity relating linearly to the aPTT time. From this the activity of the sample in the plate well is determined and then the activity in the sample is determined by multiplying by 11 to account for the dilution into the FVIII deficient plasma. By also determining the amount of FVIII antigen present in the samples (via A280 or ELISA), a specific activity of a sample can be determine to understand the relative potency of a particular preparation of FVIII. This enables the relative efficiency of different isolation strategies or construct designs for CFXTEN fusions to be ranked.

Western Blot Analysis of FVIII/FVIII-XTEN Expressed Proteins

Samples were run on a 8% homogeneous SDS gel and subsequently transferred to PVDF membrane. The samples in lanes 1-15 were: MW Standards, FVIII (42.5 ng), pBC0100B, pBC0114A, pBC0100, pBC0114, pBC0126, pBC0127 (8/5/11; #9), pBC0128, pBC0135, pBC0136, pBC0137, pBC0145, pBC0149, and pBC0146, respectively. The membrane was initially blocked with 5% milk then probed with anti-FVIII monoclonal antibody, GMA-012, specific to the A2 domain of the heavy chain (Ansong C, Miles S M, Fay P J. J Thromb Haemost. 2006 April; 4(4):842-7). Insertion of XTEN288 in the B-domain was observed for pBC0136 (lane 8, FIG. 20) and pBC0137 (lane 9, FIG. 20), whereas XTEN288 insertion at the C-terminus was observed for pBC0146 (lane 12, FIG. 20). All of the assayed FVIII-XTEN proteins revealed the presence of single chain protein with molecular weight of at least 21 kDa higher than that of pBC0114 base construct or FVIII standard. In addition, AE42 insertion was observed for pBC0135 (lane 7, FIG. 20) and pBC0149 (lane 11, FIG. 20) with the single chain running ˜5 kDa higher than that of pBC0114 base protein and heavy chain running at ˜5 kDa higher than 90 kDa band of the base protein.

### Example 23

### Pharmacokinetic Analysis of CFXTEN Fusion Polypeptides in Rats

The pharmacokinetics of various CFXTEN fusion proteins, compared to FVIII alone, are tested in Sprague-Dawley rats. CFXTEN and FVIII are administered to female Sprague-Dawley rats (n=3) IV through a jugular vein catheter at 3-10 μg/rat. Blood samples (0.2 mL) are collected into pre-chilled heparinized tubes at predose, 0.08, 0.5, 1, 2, 4, 8, 24, 48, 72 hour time points, and processed into plasma. Quantitation of the test articles is performed by ELISA assay using an anti-F VIII antibody for both capture and detection. A non-compartmental analysis is performed in WinNonLin with all time points included in the fit to determine the PK parameters. Results are expected to show increased terminal half-life and area under the curve, and a reduced volume of distribution for the CFXEN compared to FVIII alone, and the results are used in conjunction with results from coagulation and pharmacodynamic assays to select those fusion protein configurations with desired properties.

### Example 24

### Pharmacodynamic Evaluation of CFXTEN in Animal Models

The in vivo pharmacologic activity of CFXTEN fusion proteins are assessed using a variety of preclinical models of bleeding including but not limited to those of hemophilia, surgery, trauma, thrombocytopenia/platelet dysfunction, clopidogrel/heparin-induced bleeding and hydrodynamic injection. These models are developed in multiple species including mice, rat, rabbits, and dogs using methods equivalent to those used and published for other FVIII approaches. CFXTEN compositions are provided in an aqueous buffer compatible with in vivo administration (for example: phosphate-buffered saline or Tris-buffered saline). The compositions are administered at appropriate doses, dosing frequency, dosing schedule and route of administration as optimized for the particular model. Efficacy determinations include measurement of FVIII activity, one-stage clotting assay, FVIII chromogenic assay, activated partial prothrombin time (aPTT), bleeding time, whole blood clotting time (WBCT), thrombelastography (TEG or ROTEM), among others.

In one example of a PD model, CFXTEN and FVIII are administered to genetically-deficient or experimentally-induced HemA mice. At various time points post-administration, levels of FVIII and CFXTEN are measured by ELISA, activity of FVIII and CFXTEN is measured by commercially-available FVIII activity kits and clotting time is measured by aPTT assay. Overall, the results can indicate that the CFXTEN constructs may be more efficacious at inhibiting bleeding as compared to FVIII and/or equivalent in potency to comparable dosage of FVIII with less frequent or more convenient dosing intervals.

In a mouse bleeding challenge PD model CFXTEN and FVIII are administered to genetically-deficient or experimentally-induced HemA mice and effect on hemostatic challenge is measured. Hemostatic challenge can include tail transaction challenge, hemarthropthy challenge, joint bleeding or saphenous vein challenge among others. At various time points post-administration levels of FVIII and CFXTEN are measured by ELISA, activity of FVIII and CFXTEN are measured by commercially available FVIII activity kit, bleeding time is measured and clotting time is measured by aPTT assay. Overall the results are expected to indicate that the CFXTEN constructs are more efficacious at inhibiting bleeding as compared to FVIII and/or equivalent in potency to comparable dosage of FVIII with less frequent or more convenient dosing intervals, and the results are used in conjunction with results from coagulation and other assays to select those fusion protein configurations with desired properties.

In a dog PD model, CFXTEN and FVIII are administered to genetically-deficient hemophiliac dogs. At various time points post administration, levels of FVIII and CFXTEN are measured by ELISA, activity of FVIII and CFXTEN are measured by commercially available FVIII activity kit and clotting time is measured by aPTT assay. Overall the results indicates that the CFXTEN constructs may be more efficacious at inhibiting bleeding as compared to FVIII and/or equivalent in potency to comparable dosage of FVIII with less frequent or more convenient dosing, and the results are used in conjunction with results from coagulation and other assays to select those fusion protein configurations with desired properties.

In a dog bleeding challenge PD model CFXTEN and FVIII are administered to genetically deficient hemophiliac dogs and effect on hemostatic challenge is measured. Hemostatic challenge includes cuticle bleeding time among others. At various time points post-administration levels of FVIII and CFXTEN are measured by ELISA, activity of FVIII and CFXTEN are measured by commercially available FVIII activity kit, bleeding time is measured and clotting time are measured by aPTT assay. Overall the results indicate that the CFXTEN constructs may be more efficacious at inhibiting bleeding as compared to FVIII and/or equivalent in potency to comparable dosage of FVIII with less frequent or more convenient dosing intervals, and the results are used in conjunction with results from coagulation and other assays to select those fusion protein configurations with desired properties.

Additional preclinical models of bleeding include but are not limited to those of hemophilia, surgery, trauma, thrombocytopenia/platelet dysfunction, clopidogrel/heparin-induced bleeding and hydrodynamic injection. These models can developed in multiple species including mice, rat, rabbits, and dogs using methods equivalent to those used and published for other FVIII approaches. Overall the results indicate that the CFXTEN constructs may be more efficacious at inhibiting bleeding as compared to FVIII and/or equivalent in potency to comparable dosage of FVIII with less frequent or more convenient dosing intervals, and the results are used in conjunction with results from coagulation and other assays to select those fusion protein configurations with desired properties.

### Example 25

### CFXTEN with Cleavage Sequences

C-Terminal XTEN Releasable by FXIa

A CFXTEN fusion protein consisting of an XTEN protein fused to the C-terminus of FVIII is created with an XTEN release site cleavage sequence placed in between the FVIII and XTEN components, as depicted in FIG. 10. Exemplary sequences are provided in Table 30. In this case, the release site cleavage sequence is incorporated into the CFXTEN that contains an amino acid sequence that is recognized and cleaved by the FXIa protease (EC 3.4.21.27, Uniprot P03951). Specifically the amino acid sequence KLTRAET is cut after the arginine of the sequence by FXIa protease. FXI is the procoagulant protease located immediately before FVIII in the intrinsic or contact activated coagulation pathway. Active FXIa is produced from FXI by proteolytic cleavage of the zymogen by FXIIa. Production of FXIa is tightly controlled and only occurs when coagulation is necessary for proper hemostasis. Therefore, by incorporation of the KLTRAET cleavage sequence, the XTEN domain is only be removed from FVIII concurrent with activation of the intrinsic coagulation pathway and when coagulation is required physiologically. This creates a situation where the CFXTEN fusion protein is processed in one additional manner during the activation of the intrinsic pathway.

C-Terminal XTEN Releasable by FIIa (Thrombin)

A CFXTEN fusion protein consisting of an XTEN protein fused to the C-terminus of FVIII is created with an XTEN release site cleavage sequence placed in between the FVIII and XTEN components, as depicted in FIG. 10. In this case, the release site contains an amino acid sequence that is recognized and cleaved by the FIIa protease (EC 3.4.21.5, Uniprot P00734). Specifically the sequence LTPRSLLV [Rawlings N. D., et al. (2008) Nucleic Acids Res., 36: D320], is cut after the arginine at position 4 in the sequence. Active FIIa is produced by cleavage of FII by FXa in the presence of phospholipids and calcium and is down stream from factor IX in the coagulation pathway. Once activated its natural role in coagulation is to cleave fibrinogin (FIG. 2), which then in turn, begins clot formation. Ma activity is tightly controlled and only occurs when coagulation is necessary for proper hemostasis. Therefore, by incorporation of the LTPRSLLV sequence, the XTEN domain is only removed from FVIII concurrent with activation of either the extrinsic or intrinsic coagulation pathways, and when coagulation is required physiologically. This creates a situation where CFXTEN fusion is processed in one additional manner during the activation of coagulation.

C-Terminal XTEN Releasable by Elastase-2

A CFXTEN fusion protein consisting of an XTEN protein fused to the C-terminus of FVIII is created with an XTEN release site cleavage sequence placed in between the FVIII and XTEN components, as depicted in FIG. 10. Exemplary sequences are provided in Table 30. In this case, the release site contains an amino acid sequence that is recognized and cleaved by the elastase-2 protease (EC 3.4.21.37, Uniprot P08246). Specifically the sequence LGPVSGVP [Rawlings N. D., et al. (2008) Nucleic Acids Res., 36: D320], is cut after position 4 in the sequence. Elastase is constitutively expressed by neutrophils and is present at all times in the circulation. Its activity is tightly controlled by serpins and is therefore minimally active most of the time. Therefore as the long lived CFXTEN circulates, a fraction of it is cleaved, creating a pool of shorter-lived FVIII to be used in coagulation. In a desirable feature of the inventive composition, this creates a circulating pro-drug depot that constantly releases a prophylactic amount of FVIII.

C-Terminal XTEN Releasable by MMP-12

A CFXTEN fusion protein consisting of an XTEN protein fused to the C-terminus of FVIII is created with an XTEN release site cleavage sequence placed in between the FVIII and XTEN components, as depicted in FIG. 10. Exemplary sequences are provided in Table 30. In this case, the release site contains an amino acid sequence that is recognized and cleaved by the MMP-12 protease (EC 3.4.24.65, Uniprot P39900). Specifically the sequence GPAGLGGA [Rawlings N. D., et al. (2008) Nucleic Acids Res., 36: D320], is cut after position 4 of the sequence. MMP-12 is constitutively expressed in whole blood. Therefore as the long lived CFXTEN circulates, a fraction of it is cleaved, creating a pool of shorter-lived FVIII to be used in coagulation. In a desirable feature of the inventive composition, this creates a circulating pro-drug depot that constantly releases a prophylactic amount of FVIII.

C-Terminal XTEN Releasable by MMP-13

A CFXTEN fusion protein consisting of an XTEN protein fused to the C-terminus of FVIII is created with an XTEN release site cleavage sequence placed in between the FVIII and XTEN components, as depicted in FIG. 10. Exemplary sequences are provided in Table 30. In this case, the release site contains an amino acid sequence that is recognized and cleaved by the MMP-13 protease (EC 3.4.24.-, Uniprot P45452). Specifically the sequence GPAGLRGA [Rawlings N. D., et al. (2008) Nucleic Acids Res., 36: D320], is cut after position 4. MMP-13 is constitutively expressed in whole blood. Therefore as the long lived CFXTEN circulates, a fraction of it is cleaved, creating a pool of shorter-lived FVIII to be used in coagulation. In a desirable feature of the inventive composition, this creates a circulating pro-drug depot that constantly releases a prophylactic amount of FVIII.

C-Terminal XTEN Releasable by MMP-17

A CFXTEN fusion protein consisting of an XTEN protein fused to the C-terminus of FVIII is created with an XTEN release site cleavage sequence placed in between the FVIII and XTEN components, as depicted in FIG. 10. Exemplary sequences are provided in Table 30. In this case, the release site contains an amino acid sequence that is recognized and cleaved by the MMP-20 protease (EC.3.4.24.-, Uniprot Q9ULZ9). Specifically the sequence APLGLRLR [Rawlings N. D., et al. (2008) Nucleic Acids Res., 36: D320], is cut after position 4 in the sequence. MMP-17 is constitutively expressed in whole blood. Therefore as the long lived CFXTEN circulates, a fraction of it is cleaved, creating a pool of shorter-lived FVIII to be used in coagulation. In a desirable feature of the inventive composition, this creates a circulating pro-drug depot that constantly releases a prophylactic amount of FVIII.

C-Terminal XTEN Releasable by MMP-20

A CFXTEN fusion protein consisting of an XTEN protein fused to the C-terminus of FVIII is created with an XTEN release site cleavage sequence placed in between the FVIII and XTEN components, as depicted in FIG. 10. Exemplary sequences are provided in Table 30. In this case, the release site contains an amino acid sequence that is recognized and cleaved by the MMP-20 protease (EC.3.4.24.-, Uniprot 060882). Specifically the sequence PALPLVAQ [Rawlings N. D., et al. (2008) Nucleic Acids Res., 36: D320], is cut after position 4 (depicted by the arrow). MMP-20 is constitutively expressed in whole blood. Therefore as the long lived CFXTEN circulates, a fraction of it is cleaved, creating a pool of shorter-lived FVIII to be used in coagulation. In a desirable feature of the inventive composition, this creates a circulating pro-drug depot that constantly releases a prophylactic amount of FVIII.

Optimization of the Release Rate of XTEN

Variants of the foregoing Examples can be created in which the release rate of XTEN incorporated at the C-terminus, the N-terminus, or internal XTEN is altered. As the rate of XTEN release by an XTEN release protease is dependent on the sequence of the XTEN release site, by varying the amino acid sequence in the XTEN release site one can control the rate of XTEN release. The sequence specificity of many proteases is well known in the art, and is documented in several data bases. In this case, the amino acid specificity of proteases is mapped using combinatorial libraries of substrates [Harris, J. L., et al. (2000) Proc Nail Acad Sci USA, 97: 7754] or by following the cleavage of substrate mixtures as illustrated in [Schellenberger, V., et al. (1993) Biochemistry, 32: 4344]. An alternative is the identification of optimal protease cleavage sequences by phage display [Matthews, D., et al. (1993) Science, 260: 1113]. Constructs are made with variant sequences and assayed for XTEN release using standard assays for detection of the XTEN polypeptides.

### Example 26

### Human Clinical Trial Designs for Evaluating CFXTEN Comprising FVIII

Kogenate® FS is recombinant human coagulation factor VIII, intended for promoting hemostasis in hemophilia A subjects. Due to its short half-life, Kogenate is dosed intravenously every other day for prophylaxis and 8 to every 12 h in treatment of bleeds until hemostasis is achieved. It is believed that fusion of XTEN to FVIII improves the half-life of the protein, enabling a reduced dosing frequency using such CFXTEN-containing fusion protein compositions.

Clinical trials are designed such that the efficacy and advantages of CFXTEN, relative to Kogenate, can be verified in humans. For example, the CFXTEN is used in clinical trials for treatment of bleeding as performed for Kogenate. Such studies comprises three phases. First, a Phase I safety and pharmacokinetics study in adult patients is conducted to determine the maximum tolerated dose and pharmacokinetics and pharmacodynamics in humans (either normal subjects or patients with hemophilia), as well as to define potential toxicities and adverse events to be tracked in future studies. The Phase I studies are conducted in which single rising doses of CFXTEN compositions are administered by the route (e.g., subcutaneous, intramuscular, or intravenously) and biochemical, PK, and clinical parameters are measured at defined intervals. This permits the determination of the minimum effective dose and the maximum tolerated dose and establishes the threshold and maximum concentrations in dosage and circulating drug that constitute the therapeutic window for the respective components, as well as bioavailability when administered by the intramuscular or subcutaneous routes. From this information, the dose and dose schedule that permits less frequent administration of the CFXTEN compositions, yet retains the pharmacologic response, is obtained. Thereafter, clinical trials are conducted in patients with the disease, disorder or condition, verifying the effectiveness of the CFXTEN compositions under the dose conditions, which can be conducted in comparison to a positive control such as Kogenate to establish the enhanced properties of the CFXTEN compositions.

Clinical trials are conducted in patients suffering from any disease in which Kogenate may be expected to provide clinical benefit. For example, such indications include bleeding episodes in hemophilia A, patients with inhibitors to factor VIII, prevention of bleeding in surgical interventions or invasive procedures in hemophilia A patients with inhibitors to factor VIII, treatment of bleeding episodes in patients with congenital FVIII deficiency, and prevention of bleeding in surgical interventions or invasive procedures in patients with congenital FVIII deficiency. CFXTEN may also be indicated for use in additional patient populations. Parameters and clinical endpoints are measured as a function of the dosing of the fusion proteins compositions, yielding dose-ranging information on doses that is appropriate for a subsequent Phase III trial, in addition to collecting safety data related to adverse events. The PK parameters are correlated to the physiologic, clinical and safety parameter data to establish the therapeutic window and the therapeutic dose regimen for the CFXTEN composition, permitting the clinician to establish the appropriate dose ranges for the composition. Finally, a phase III efficacy study is conducted wherein patients is administered the CFXTEN composition at the dose regimen, and a positive control (such as a commercially-available Kogenate), or a placebo is administered using a dosing schedule deemed appropriate given the pharmacokinetic and pharmacodynamic properties of the respective compositions, with all agents administered for an appropriately extended period of time to achieve the study endpoints. Parameters that are monitored include aPTT assay, one- or two-stage clotting assays, control of bleeding episodes, or the occurrence of spontaneous bleeding episodes; parameters that are tracked relative to the placebo or positive control groups. Efficacy outcomes are determined using standard statistical methods. Toxicity and adverse event markers are also be followed in this study to verify that the compound is safe when used in the manner described.

### Example 27

### Analytical Size Exclusion Chromatography of XTEN Fusion Proteins with Diverse Payloads

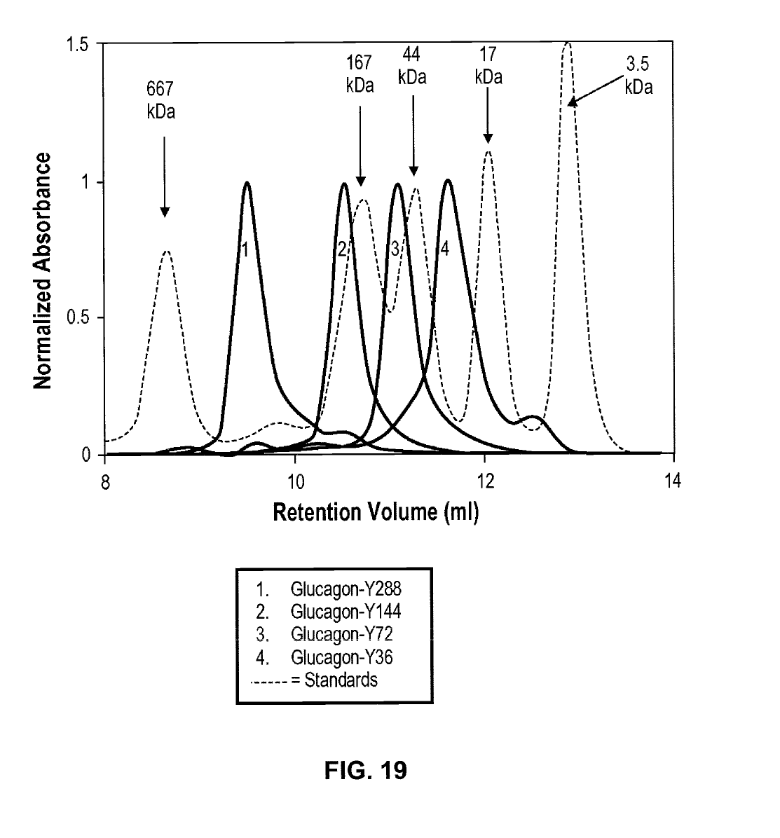

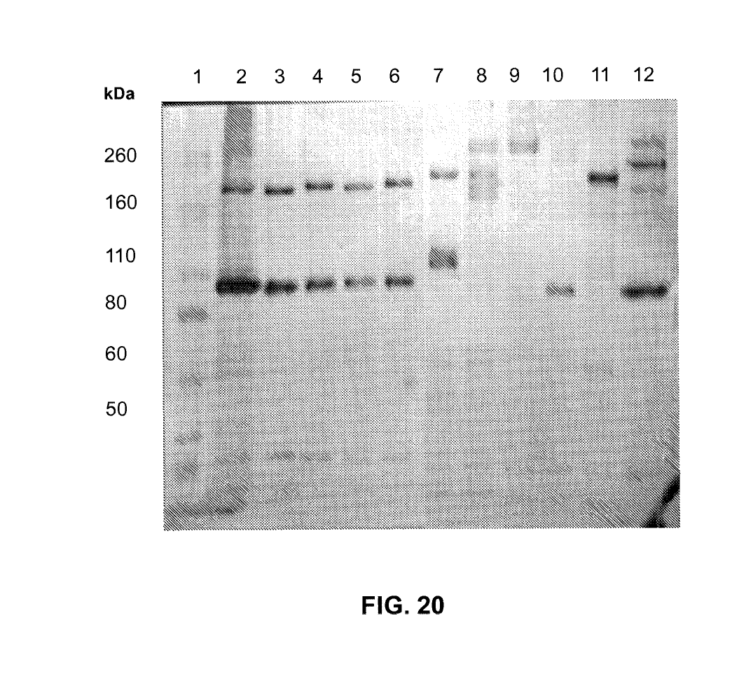

Size exclusion chromatography analyses were performed on fusion proteins containing various therapeutic proteins and unstructured recombinant proteins of increasing length. An exemplary assay used a TSKGel-G4000 SWXL (7.8 mm×30 cm) column in which 40 μg of purified glucagon fusion protein at a concentration of 1 mg/ml was separated at a flow rate of 0.6 ml/min in 20 mM phosphate pH 6.8, 114 mM NaCl. Chromatogram profiles were monitored using OD214 nm and OD280 nm. Column calibration for all assays were performed using a size exclusion calibration standard from BioRad; the markers include thyroglobulin (670 kDa), bovine gamma-globulin (158 kDa), chicken ovalbumin (44 kDa), equine myoglobuin (17 kDa) and vitamin B12 (1.35 kDa). Representative chromatographic profiles of Glucagon-Y288, Glucagon-Y144, Glucagon-Y72, Glucagon-Y36 are shown as an overlay in FIG. 19. The data show that the apparent molecular weight of each compound is proportional to the length of the attached XTEN sequence. However, the data also show that the apparent molecular weight of each construct is significantly larger than that expected for a globular protein (as shown by comparison to the standard proteins run in the same assay). Based on the SEC analyses for all constructs evaluated, including a CFXTEN composition, the apparent molecular weights, the apparent molecular weight factor (expressed as the ratio of apparent molecular weight to the calculated molecular weight) and the hydrodynamic radius (R_{H} in nm) are shown in Table 16. The results indicate that incorporation of different XTENs of 576 amino acids or greater confers an apparent molecular weight for the fusion protein of approximately 339 kDa to 760, and that XTEN of 864 amino acids or greater confers an apparent molecular weight greater than approximately 800 kDA. The results of proportional increases in apparent molecular weight to actual molecular weight were consistent for fusion proteins created with XTEN from several different motif families; i.e., AD, AE, AF, AG, and AM, with increases of at least four-fold and ratios as high as about 17-fold. Additionally, the incorporation of XTEN fusion partners with 576 amino acids or more into fusion proteins with the various payloads (and 288 residues in the case of glucagon fused to Y288) resulted with a hydrodynamic radius of 7 nm or greater, well beyond the glomerular pore size of approximately 3-5 nm. Accordingly, it is expected that fusion proteins comprising growth and XTEN have reduced renal clearance, contributing to increased terminal half-life and improving the therapeutic or biologic effect relative to a corresponding un-fused biologic payload protein.

TABLE 16
SEC analysis of various polypeptides

| | | | | | Apparent | |
|---|---|---|---|---|---|---|
| Con- | XTEN or | Thera- | Actual | Apparent | Molecular | |
| struct | fusion | peutic | MW | MW | Weight | R_{H} |
| Name | partner | Protein | (kDa) | (kDa) | Factor | (nm) |
| AC14 | Y288 | Glucagon | 28.7 | 370 | 12.9 | 7.0 |
| AC28 | Y144 | Glucagon | 16.1 | 117 | 7.3 | 5.0 |
| AC34 | Y72 | Glucagon | 9.9 | 58.6 | 5.9 | 3.8 |
| AC33 | Y36 | Glucagon | 6.8 | 29.4 | 4.3 | 2.6 |
| AC89 | AF120 | Glucagon | 14.1 | 76.4 | 5.4 | 4.3 |
| AC88 | AF108 | Glucagon | 13.1 | 61.2 | 4.7 | 3.9 |
| AC73 | AF144 | Glucagon | 16.3 | 95.2 | 5.8 | 4.7 |
| AC53 | AG576 | GFP | 74.9 | 339 | 4.5 | 7.0 |
| AC39 | AD576 | GFP | 76.4 | 546 | 7.1 | 7.7 |
| AC41 | AE576 | GFP | 80.4 | 760 | 9.5 | 8.3 |
| AC52 | AF576 | GFP | 78.3 | 526 | 6.7 | 7.6 |
| AC398 | AE288 | FVII | 76.3 | 650 | 8.5 | 8.2 |
| AC404 | AE864 | FVII | 129 | 1900 | 14.7 | 10.1 |
| AC85 | AE864 | Exendin-4 | 83.6 | 938 | 11.2 | 8.9 |
| AC114 | AM875 | Exendin-4 | 82.4 | 1344 | 16.3 | 9.4 |
| AC143 | AM875 | CF | 100.6 | 846 | 8.4 | 8.7 |
| AC227 | AM875 | IL-1ra | 95.4 | 1103 | 11.6 | 9.2 |
| AC228 | AM1318 | IL-1ra | 134.8 | 2286 | 17.0 | 10.5 |

### Example 28

### Pharmacokinetics of Extended Polypeptides Fused to GFP in Cynomolgus Monkeys

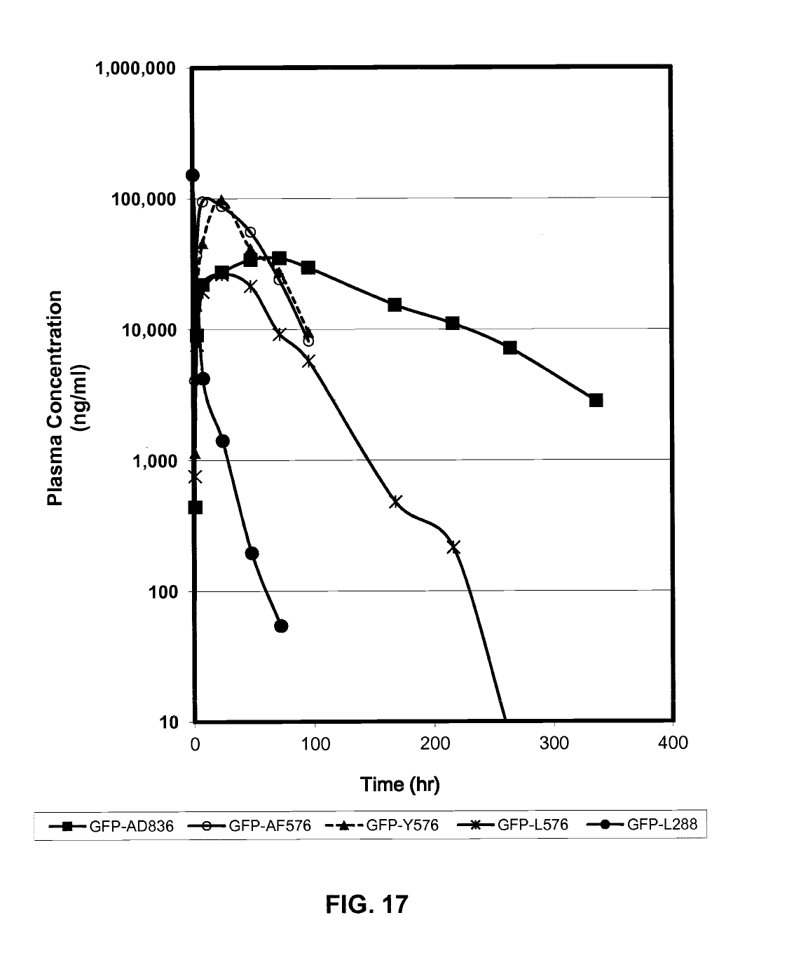

The pharmacokinetics of GFP-L288, GFP-L576, GFP-XTEN_AF576, GFP-XTEN_Y576 and XTEN_AD836-GFP were tested in cynomolgus monkeys to determine the effect of composition and length of the unstructured polypeptides on PK parameters. Blood samples were analyzed at various times after injection and the concentration of GFP in plasma was measured by ELISA using a polyclonal antibody against GFP for capture and a biotinylated preparation of the same polyclonal antibody for detection. Results are summarized in FIG. 17. They show a surprising increase of half-life with increasing length of the XTEN sequence. For example, a half-life of 10 h was determined for GFP-XTEN_L288 (with 288 amino acid residues in the XTEN). Doubling the length of the unstructured polypeptide fusion partner to 576 amino acids increased the half-life to 20-22 h for multiple fusion protein constructs; i.e., GFP-XTEN_L576, GFP-XTEN_AF576, GFP-XTEN_Y576. A further increase of the unstructured polypeptide fusion partner length to 836 residues resulted in a half-life of 72-75 h for XTEN_AD836-GFP. Thus, increasing the polymer length by 288 residues from 288 to 576 residues increased in vivo half-life by about 10 h. However, increasing the polypeptide length by 260 residues from 576 residues to 836 residues increased half-life by more than 50 h. These results show that there is a surprising threshold of unstructured polypeptide length that results in a greater than proportional gain in in vivo half-life. Thus, fusion proteins comprising extended, unstructured polypeptides are expected to have the property of enhanced pharmacokinetics compared to polypeptides of shorter lengths.

### Example 29

### Serum Stability of XTEN

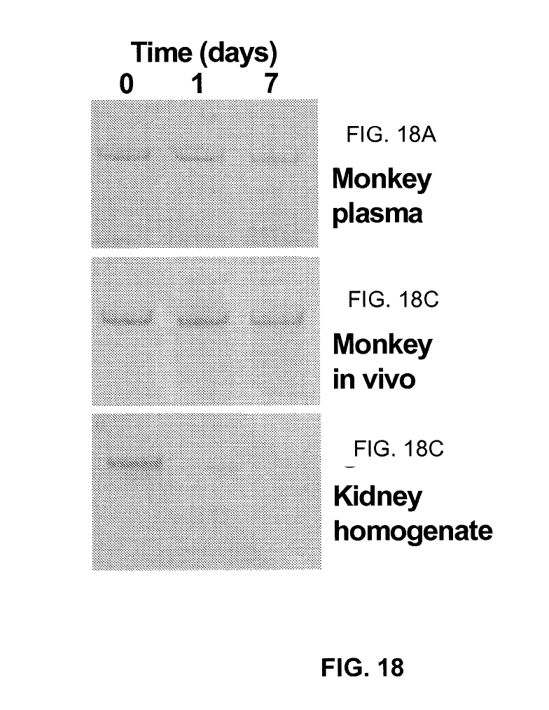

A fusion protein containing XTEN_AE864 fused to the N-terminus of GFP was incubated in monkey plasma and rat kidney lysate for up to 7 days at 37° C. Samples were withdrawn at time 0, Day 1 and Day 7 and analyzed by SDS PAGE followed by detection using Western analysis and detection with antibodies against GFP as shown in FIG. 18. The sequence of XTEN_AE864 showed negligible signs of degradation over 7 days in plasma. However, XTEN_AE864 was rapidly degraded in rat kidney lysate over 3 days. The in vivo stability of the fusion protein was tested in plasma samples wherein the GFP_AE864 was immunoprecipitated and analyzed by SDS PAGE as described above. Samples that were withdrawn up to 7 days after injection showed very few signs of degradation. The results demonstrate the resistance of CFXTEN to degradation due to serum proteases; a factor in the enhancement of pharmacokinetic properties of the CFXTEN fusion proteins.

### Example 30

### Increasing Solubility and Stability of a Peptide Payload by Linking to XTEN

In order to evaluate the ability of XTEN to enhance the physicochemical properties of solubility and stability, fusion proteins of glucagon plus shorter-length XTEN were prepared and evaluated. The test articles were prepared in Tris-buffered saline at neutral pH and characterization of the Gcg-XTEN solution was by reverse-phase HPLC and size exclusion chromatography to affirm that the protein was homogeneous and non-aggregated in solution. The data are presented in Table 17. For comparative purposes, the solubility limit of unmodified glucagon in the same buffer was measured at 60 μM (0.2 mg/mL), and the result demonstrate that for all lengths of XTEN added, a substantial increase in solubility was attained. Importantly, in most cases the glucagon-XTEN fusion proteins were prepared to achieve target concentrations and were not evaluated to determine the maximum solubility limits for the given construct. However, in the case of glucagon linked to the AF-144×TEN, the limit of solubility was determined, with the result that a 60-fold increase in solubility was achieved, compared to glucagon not linked to XTEN. In addition, the glucagon-AF144 CFXTEN was evaluated for stability, and was found to be stable in liquid formulation for at least 6 months under refrigerated conditions and for approximately one month at 37° C. (data not shown).

The data support the conclusion that the linking of short-length XTEN polypeptides to a biologically active protein such as glucagon can markedly enhance the solubility properties of the protein by the resulting fusion protein, as well as confer stability at the higher protein concentrations.

TABLE 17
Solubility of Glucagon-XTEN constructs

| Test Article | Solubility |
|---|---|
| Glucagon | 60 μM |
| Glucagon-Y36 | >370 μM |
| Glucagon-Y72 | >293 μM |
| Glucagon-AF108 | >145 μM |
| Glucagon-AF120 | >160 μM |
| Glucagon-Y144 | >497 μM |
| Glucagon-AE144 | >467 μM |
| Glucagon-AF144 | >3600 μM |
| Glucagon-Y288 | >163 μM |

### Example 31

### Analysis of Sequences for Secondary Structure by Prediction Algorithms

Amino acid sequences can be assessed for secondary structure via certain computer programs or algorithms, such as the well-known Chou-Fasman algorithm (Chou, P. Y., et al. (1974) Biochemistry, 13: 222-45) and the Garnier-Osguthorpe-Robson, or “GOR” method (Garnier J, Gibrat J F, Robson B. (1996). GOR method for predicting protein secondary structure from amino acid sequence. Methods Enzymol 266:540-553). For a given sequence, the algorithms can predict whether there exists some or no secondary structure at all, expressed as total and/or percentage of residues of the sequence that form, for example, alpha-helices or beta-sheets or the percentage of residues of the sequence predicted to result in random coil formation.

Several representative sequences from XTEN “families” have been assessed using two algorithm tools for the Chou-Fasman and GOR methods to assess the degree of secondary structure in these sequences. The Chou-Fasman tool was provided by William R. Pearson and the University of Virginia, at the “Biosupport” internet site, URL located on the World Wide Web at .fasta.bioch.virginia.edu/fasta_www2/fasta_www.cgi?rm=misc1 as it existed on Jun. 19, 2009. The GOR tool was provided by Pole Informatique Lyonnais at the Network Protein Sequence Analysis internet site, URL located on the World Wide Web at .npsa-pbil.ibcp.fr/cgi-bin/secpred_gor4.pl as it existed on Jun. 19, 2008.

As a first step in the analyses, a single XTEN sequence was analyzed by the two algorithms. The AE864 composition is an XTEN with 864 amino acid residues created from multiple copies of four 12 amino acid sequence motifs consisting of the amino acids G, S, T, E, P, and A. The sequence motifs are characterized by the fact that there is limited repetitiveness within the motifs and within the overall sequence in that the sequence of any two consecutive amino acids is not repeated more than twice in any one 12 amino acid motif, and that no three contiguous amino acids of full-length the XTEN are identical. Successively longer portions of the AF 864 sequence from the N-terminus were analyzed by the Chou-Fasman and GOR algorithms (the latter requires a minimum length of 17 amino acids). The sequences were analyzed by entering the FASTA format sequences into the prediction tools and running the analysis. The results from the analyses are presented in Table 18.

The results indicate that, by the Chou-Fasman calculations, short XTEN of the AE and AG families, up to at least 288 amino acid residues, have no alpha-helices or beta-sheets, but amounts of predicted percentage of random coil by the GOR algorithm vary from 78-99%. With increasing XTEN lengths of 504 residues to greater than 1300, the XTEN analyzed by the Chou-Fasman algorithm had predicted percentages of alpha-helices or beta-sheets of 0 to about 2%, while the calculated percentages of random coil increased to from 94-99%. Those XTEN with alpha-helices or beta-sheets were those sequences with one or more instances of three contiguous serine residues, which resulted in predicted beta-sheet formation. However, even these sequences still had approximately 99% random coil formation.

The data provided herein suggests that 1) XTEN created from multiple sequence motifs of G, S, T, E, P, and A that have limited repetitiveness as to contiguous amino acids are predicted to have very low amounts of alpha-helices and beta-sheets; 2) that increasing the length of the XTEN does not appreciably increase the probability of alpha-helix or beta-sheet formation; and 3) that progressively increasing the length of the XTEN sequence by addition of non-repetitive 12-mers consisting of the amino acids G, S, T, E, P, and A results in increased percentage of random coil formation. Results further indicate that XTEN sequences defined herein (including e.g., XTEN created from sequence motifs of G, S, T, E, P, and A) have limited repetitiveness (including those with no more than two identical contiguous amino acids in any one motif) are expected to have very limited secondary structure. Any order or combination of sequence motifs from Table 3 can be used to create an XTEN polypeptide that will result in an XTEN sequence that is substantially devoid of secondary structure, though three contiguous serines are not preferred. The unfavorable property of three contiguous series however, can be ameliorated by increasing the length of the XTEN. Such sequences are expected to have the characteristics described in the CFXTEN embodiments of the invention disclosed herein.

TABLE 18
CHOU-FASMAN and GOR prediction calculations of polypeptide sequences

| SEQ | | No. | Chou-Fasman | GOR |
|---|---|---|---|---|
| NAME | Sequence | Residues | Calculation | Calculation |
| AE36: | GSPAGSPTSTEEGTSESATPESGPGT | 36 | Residue totals: H: 0 E: 0 | 94.44% |
| LCW0402_002 | STEPSEGSAP | | percent: H: 0.0 E: 0.0 | |
| AE36: | GTSTEPSEGSAPGTSTEPSEGSAPGT | 36 | Residue totals: H: 0 E: 0 | 94.44% |
| LCW0402_003 | STEPSEGSAP | | percent: H: 0.0 E: 0.0 | |
| AG36: | GASPGTSSTGSPGTPGSGTASSSPGS | 36 | Residue totals: H: 0 E: 0 | 77.78% |
| LCW0404_001 | STPSGATGSP | | percent: H: 0.0 E: 0.0 | |
| AG36: | GSSTPSGATGSPGSSPSASTGTGPGS | 36 | Residue totals: H: 0 E: 0 | 83.33% |
| LCW0404_003 | STPSGATGSP | | percent: H: 0.0 E: 0.0 | |
| AE42_1 | TEPSEGSAPGSPAGSPTSTEEGTSES | 42 | Residue totals: H: 0 E: 0 | 90.48% |
| | ATPESGPGSEPATSGS | | percent: H: 0.0 E: 0.0 | |
| AE42_1 | TEPSEGSAPGSPAGSPTSTEEGTSES | 42 | Residue totals: H: 0 E: 0 | 90.48% |
| | ATPESGPGSEPATSGS | | percent: H: 0.0 E: 0.0 | |
| AG42_1 | GAPSPSASTGTGPGTPGSGTASSSPG | 42 | Residue totals: H: 0 E: 0 | 88.10% |
| | SSTPSGATGSPGPSGP | | percent: H: 0.0 E: 0.0 | |
| AG42_2 | GPGTPGSGTASSSPGSSTPSGATGSP | 42 | Residue totals: H: 0 E: 0 | 88.10% |
| | GSSPSASTGTGPGASP | | percent: H: 0.0 E: 0.0 | |
| AE144 | GSEPATSGSETPGTSESATPESGPGS | 144 | Residue totals: H: 0 E: 0 | 98.61% |
| | EPATSGSETPGSPAGSPTSTEEGTST | | percent: H: 0.0 E: 0.0 | |
| | EPSEGSAPGSEPATSGSETPGSEPAT | | | |
| | SGSETPGSEPATSGSETPGTSTEPSE | | | |
| | GSAPGTSESATPESGPGSEPATSGSE | | | |
| | TPGTSTEPSEGSAP | | | |
| AG144_1 | PGSSPSASTGTGPGSSPSASTGTGPG | 144 | Residue totals: H: 0 E: 0 | 91.67% |
| | TPGSGTASSSPGSSTPSGATGSPGSS | | percent: H: 0.0 E: 0.0 | |
| | PSASTGTGPGASPGTSSTGSPGTPGS | | | |
| | GTASSSPGSSTPSGATGSPGTPGSGT | | | |
| | ASSSPGASPGTSSTGSPGASPGTSST | | | |
| | GSPGTPGSGTASSS | | | |
| AE288 | GTSESATPESGPGSEPATSGSETPGT | 288 | Residue totals: H: 0 E: 0 | 99.31% |
| | SESATPESGPGSEPATSGSETPGTSE | | percent: H: 0.0 E: 0.0 | |
| | SATPESGPGTSTEPSEGSAPGSPAGS | | | |
| | PTSTEEGTSESATPESGPGSEPATSG | | | |
| | SETPGTSESATPESGPGSPAGSPTST | | | |
| | EEGSPAGSPTSTEEGTSTEPSEGSAP | | | |
| | GTSESATPESGPGTSESATPESGPGT | | | |
| | SESATPESGPGSEPATSGSETPGSEP | | | |
| | ATSGSETPGSPAGSPTSTEEGTSTEP | | | |
| | SEGSAPGTSTEPSEGSAPGSEPATSG | | | |
| | SETPGTSESATPESGPGTSTEPSEGS | | | |
| | AP | | | |
| AG288_2 | GSSPSASTGTGPGSSPSASTGTGPGT | 288 | Residue totals: H: 0 E: 0 | 92.71 |
| | PGSGTASSSPGSSTPSGATGSPGSSP | | percent: H: 0.0 E: 0.0 | |
| | SASTGTGPGASPGTSSTGSPGTPGSG | | | |
| | TASSSPGSSTPSGATGSPGTPGSGTA | | | |
| | SSSPGASPGTSSTGSPGASPGTSSTG | | | |
| | SPGTPGSGTASSSPGSSTPSGATGSP | | | |
| | GASPGTSSTGSPGTPGSGTASSSPGS | | | |
| | STPSGATGSPGSSPSASTGTGPGSSP | | | |
| | SASTGTGPGSSTPSGATGSPGSSTPS | | | |
| | GATGSPGASPGTSSTGSPGASPGTSS | | | |
| | TGSPGASPGTSSTGSPGTPGSGTASS | | | |
| | SP | | | |
| AF504 | GASPGTSSTGSPGSSPSASTGTGPGS | 504 | Residue totals: H: 0 E: 0 | 94.44% |
| | SPSASTGTGPGTPGSGTASSSPGSST | | percent: H: 0.0 E: 0.0 | |
| | PSGATGSPGSNPSASTGTGPGASPG | | | |
| | TSSTGSPGTPGSGTASSSPGSSTPSG | | | |
| | ATGSPGTPGSGTASSSPGASPGTSST | | | |
| | GSPGASPGTSSTGSPGTPGSGTASSS | | | |
| | PGSSTPSGATGSPGASPGTSSTGSPG | | | |
| | TPGSGTASSSPGSSTPSGATGSPGSN | | | |
| | PSASTGTGPGSSPSASTGTGPGSSTP | | | |
| | SGATGSPGSSTPSGATGSPGASPGTS | | | |
| | STGSPGASPGTSSTGSPGASPGTSST | | | |
| | GSPGTPGSGTASSSPGASPGTSSTGS | | | |
| | PGASPGTSSTGSPGASPGTSSTGSPG | | | |
| | SSPSASTGTGPGTPGSGTASSSPGAS | | | |
| | PGTSSTGSPGASPGTSSTGSPGASPG | | | |
| | TSSTGSPGSSTPSGATGSPGSSTPSG | | | |
| | ATGSPGASPGTSSTGSPGTPGSGTAS | | | |
| | SSPGSSTPSGATGSPGSSTPSGATGS | | | |
| | PGSSTPSGATGSPGSSPSASTGTGPG | | | |
| | ASPGTSSTGSP | | | |
| AD 576 | GSSESGSSEGGPGSGGEPSESGSSGS | 576 | Residue totals: H: 7 E: 0 | 99.65% |
| | SESGSSEGGPGSSESGSSEGGPGSSE | | percent: H: 1.2 E: 0.0 | |
| | SGSSEGGPGSSESGSSEGGPGSSESG | | | |
| | SSEGGPGESPGGSSGSESGSEGSSGP | | | |
| | GESSGSSESGSSEGGPGSSESGSSEG | | | |
| | GPGSSESGSSEGGPGSGGEPSESGSS | | | |
| | GESPGGSSGSESGESPGGSSGSESGS | | | |
| | GGEPSESGSSGSSESGSSEGGPGSGG | | | |
| | EPSESGSSGSGGEPSESGSSGSEGSS | | | |
| | GPGESSGESPGGSSGSESGSGGEPSE | | | |
| | SGSSGSGGEPSESGSSGSGGEPSESG | | | |
| | SSGSSESGSSEGGPGESPGGSSGSES | | | |
| | GESPGGSSGSESGESPGGSSGSESGE | | | |
| | SPGGSSGSESGESPGGSSGSESGSSE | | | |
| | SGSSEGGPGSGGEPSESGSSGSEGSS | | | |
| | GPGESSGSSESGSSEGGPGSGGEPSE | | | |
| | SGSSGSSESGSSEGGPGSGGEPSESG | | | |
| | SSGESPGGSSGSESGESPGGSSGSES | | | |
| | GSSESGSSEGGPGSGGEPSESGSSGS | | | |
| | SESGSSEGGPGSGGEPSESGSSGSGG | | | |
| | EPSESGSSGESPGGSSGSESGSEGSS | | | |
| | GPGESSGSSESGSSEGGPGSEGSSGP | | | |
| | GESS | | | |
| AE576 | GSPAGSPTSTEEGTSESATPESGPGT | 576 | Residue totals: H: 2 E: 0 | 99.65% |
| | STEPSEGSAPGSPAGSPTSTEEGTST | | percent: H: 0.4 E: 0.0 | |
| | EPSEGSAPGTSTEPSEGSAPGTSESA | | | |
| | TPESGPGSEPATSGSETPGSEPATSG | | | |
| | SETPGSPAGSPTSTEEGTSESATPES | | | |
| | GPGTSTEPSEGSAPGTSTEPSEGSAP | | | |
| | GSPAGSPTSTEEGTSTEPSEGSAPGT | | | |
| | STEPSEGSAPGTSESATPESGPGTST | | | |
| | EPSEGSAPGTSESATPESGPGSEPAT | | | |
| | SGSETPGTSTEPSEGSAPGTSTEPSE | | | |
| | GSAPGTSESATPESGPGTSESATPES | | | |
| | GPGSPAGSPTSTEEGTSESATPESGP | | | |
| | GSEPATSGSETPGTSESATPESGPGT | | | |
| | STEPSEGSAPGTSTEPSEGSAPGTST | | | |
| | EPSEGSAPGTSTEPSEGSAPGTSTEP | | | |
| | SEGSAPGTSTEPSEGSAPGSPAGSPT | | | |
| | STEEGTSTEPSEGSAPGTSESATPES | | | |
| | GPGSEPATSGSETPGTSESATPESGP | | | |
| | GSEPATSGSETPGTSESATPESGPGT | | | |
| | STEPSEGSAPGTSESATPESGPGSPA | | | |
| | GSPTSTEEGSPAGSPTSTEEGSPAGS | | | |
| | PTSTEEGTSESATPESGPGTSTEPSE | | | |
| | GSAP | | | |
| AG576 | PGTPGSGTASSSPGSSTPSGATGSPG | 576 | Residue totals: H: 0 E: 3 | 99.31% |
| | SSPSASTGTGPGSSPSASTGTGPGSS | | percent: H: 0.4 E: 0.5 | |
| | TPSGATGSPGSSTPSGATGSPGASPG | | | |
| | TSSTGSPGASPGTSSTGSPGASPGTS | | | |
| | STGSPGTPGSGTASSSPGASPGTSST | | | |
| | GSPGASPGTSSTGSPGASPGTSSTGS | | | |
| | PGSSPSASTGTGPGTPGSGTASSSPG | | | |
| | ASPGTSSTGSPGASPGTSSTGSPGAS | | | |
| | PGTSSTGSPGSSTPSGATGSPGSSTPS | | | |
| | GATGSPGASPGTSSTGSPGTPGSGT | | | |
| | ASSSPGSSTPSGATGSPGSSTPSGAT | | | |
| | GSPGSSTPSGATGSPGSSPSASTGTG | | | |
| | PGASPGTSSTGSPGASPGTSSTGSPG | | | |
| | TPGSGTASSSPGASPGTSSTGSPGAS | | | |
| | PGTSSTGSPGASPGTSSTGSPGASPG | | | |
| | TSSTGSPGTPGSGTASSSPGSSTPSG | | | |
| | ATGSPGTPGSGTASSSPGSSTPSGAT | | | |
| | GSPGTPGSGTASSSPGSSTPSGATGS | | | |
| | PGSSTPSGATGSPGSSPSASTGTGPG | | | |
| | SSPSASTGTGPGASPGTSSTGSPGTP | | | |
| | GSGTASSSPGSSTPSGATGSPGSSPS | | | |
| | ASTGTGPGSSPSASTGTGPGASPGTS | | | |
| | STGS | | | |
| AF540 | GSTSSTAESPGPGSTSSTAESPGPGS | 540 | Residue totals: H: 2 E: 0 | 99.65 |
| | TSESPSGTAPGSTSSTAESPGPGSTSS | | percent: H: 0.4 E: 0.0 | |
| | TAESPGPGTSTPESGSASPGSTSESPS | | | |
| | GTAPGTSPSGESSTAPGSTSESPSGT | | | |
| | APGSTSESPSGTAPGTSPSGESSTAP | | | |
| | GSTSESPSGTAPGSTSESPSGTAPGT | | | |
| | SPSGESSTAPGSTSESPSGTAPGSTSE | | | |
| | SPSGTAPGSTSESPSGTAPGTSTPES | | | |
| | GSASPGSTSESPSGTAPGTSTPESGS | | | |
| | ASPGSTSSTAESPGPGSTSSTAESPG | | | |
| | PGTSTPESGSASPGTSTPESGSASPG | | | |
| | STSESPSGTAPGTSTPESGSASPGTST | | | |
| | PESGSASPGSTSESPSGTAPGSTSESP | | | |
| | SGTAPGSTSESPSGTAPGSTSSTAES | | | |
| | PGPGTSTPESGSASPGTSTPESGSAS | | | |
| | PGSTSESPSGTAPGSTSESPSGTAPG | | | |
| | TSTPESGSASPGSTSESPSGTAPGSTS | | | |
| | ESPSGTAPGTSTPESGSASPGTSPSG | | | |
| | ESSTAPGSTSSTAESPGPGTSPSGESS | | | |
| | TAPGSTSSTAESPGPGTSTPESGSAS | | | |
| | PGSTSESPSGTAP | | | |
| AD836 | GSSESGSSEGGPGSSESGSSEGGPGE | 836 | Residue totals: H: 0 E: 0 | 98.44% |
| | SPGGSSGSESGSGGEPSESGSSGESP | | percent: H: 0.0 E: 0.0 | |
| | GGSSGSESGESPGGSSGSESGSSESG | | | |
| | SSEGGPGSSESGSSEGGPGSSESGSS | | | |
| | EGGPGESPGGSSGSESGESPGGSSGS | | | |
| | ESGESPGGSSGSESGSSESGSSEGGP | | | |
| | GSSESGSSEGGPGSSESGSSEGGPGS | | | |
| | SESGSSEGGPGSSESGSSEGGPGSSE | | | |
| | SGSSEGGPGSGGEPSESGSSGESPGG | | | |
| | SSGSESGESPGGSSGSESGSGGEPSE | | | |
| | SGSSGSEGSSGPGESSGSSESGSSEG | | | |
| | GPGSGGEPSESGSSGSEGSSGPGESS | | | |
| | GSSESGSSEGGPGSGGEPSESGSSGE | | | |
| | SPGGSSGSESGSGGEPSESGSSGSGG | | | |
| | EPSESGSSGSSESGSSEGGPGSGGEP | | | |
| | SESGSSGSGGEPSESGSSGSEGSSGP | | | |
| | GESSGESPGGSSGSESGSEGSSGPGE | | | |
| | SSGSEGSSGPGESSGSGGEPSESGSS | | | |
| | GSSESGSSEGGPGSSESGSSEGGPGE | | | |
| | SPGGSSGSESGSGGEPSESGSSGSEG | | | |
| | SSGPGESSGESPGGSSGSESGSEGSS | | | |
| | GPGSSESGSSEGGPGSGGEPSESGSS | | | |
| | GSEGSSGPGESSGSEGSSGPGESSGS | | | |
| | EGSSGPGESSGSGGEPSESGSSGSGG | | | |
| | EPSESGSSGESPGGSSGSESGESPGG | | | |
| | SSGSESGSGGEPSESGSSGSEGSSGP | | | |
| | GESSGESPGGSSGSESGSSESGSSEG | | | |
| | GPGSSESGSSEGGPGSSESGSSEGGP | | | |
| | GSGGEPSESGSSGSSESGSSEGGPGE | | | |
| | SPGGSSGSESGSGGEPSESGSSGSSE | | | |
| | SGSSEGGPGESPGGSSGSESGSGGEP | | | |
| | SESGSSGESPGGSSGSESGSGGEPSE | | | |
| | SGSS | | | |
| AE864 | GSPAGSPTSTEEGTSESATPESGPGT | 864 | Residue totals: H: 2 E: 3 | 99.77% |
| | STEPSEGSAPGSPAGSPTSTEEGTST | | percent: H: 0.2 E: 0.4 | |
| | EPSEGSAPGTSTEPSEGSAPGTSESA | | | |
| | TPESGPGSEPATSGSETPGSEPATSG | | | |
| | SETPGSPAGSPTSTEEGTSESATPES | | | |
| | GPGTSTEPSEGSAPGTSTEPSEGSAP | | | |
| | GSPAGSPTSTEEGTSTEPSEGSAPGT | | | |
| | STEPSEGSAPGTSESATPESGPGTST | | | |
| | EPSEGSAPGTSESATPESGPGSEPAT | | | |
| | SGSETPGTSTEPSEGSAPGTSTEPSE | | | |
| | GSAPGTSESATPESGPGTSESATPES | | | |
| | GPGSPAGSPTSTEEGTSESATPESGP | | | |
| | GSEPATSGSETPGTSESATPESGPGT | | | |
| | STEPSEGSAPGTSTEPSEGSAPGTST | | | |
| | EPSEGSAPGTSTEPSEGSAPGTSTEP | | | |
| | SEGSAPGTSTEPSEGSAPGSPAGSPT | | | |
| | STEEGTSTEPSEGSAPGTSESATPES | | | |
| | GPGSEPATSGSETPGTSESATPESGP | | | |
| | GSEPATSGSETPGTSESATPESGPGT | | | |
| | STEPSEGSAPGTSESATPESGPGSPA | | | |
| | GSPTSTEEGSPAGSPTSTEEGSPAGS | | | |
| | PTSTEEGTSESATPESGPGTSTEPSE | | | |
| | GSAPGTSESATPESGPGSEPATSGSE | | | |
| | TPGTSESATPESGPGSEPATSGSETP | | | |
| | GTSESATPESGPGTSTEPSEGSAPGS | | | |
| | PAGSPTSTEEGTSESATPESGPGSEP | | | |
| | ATSGSETPGTSESATPESGPGSPAGS | | | |
| | PTSTEEGSPAGSPTSTEEGTSTEPSE | | | |
| | GSAPGTSESATPESGPGTSESATPES | | | |
| | GPGTSESATPESGPGSEPATSGSETP | | | |
| | GSEPATSGSETPGSPAGSPTSTEEGT | | | |
| | STEPSEGSAPGTSTEPSEGSAPGSEP | | | |
| | ATSGSETPGTSESATPESGPGTSTEP | | | |
| | SEGSAP | | | |
| AF864 | GSTSESPSGTAPGTSPSGESSTAPGS | 875 | Residue totals: H: 2 E: 0 | 95.20% |
| | TSESPSGTAPGSTSESPSGTAPGTSTP | | percent: H: 0.2 E: 0.0 | |
| | ESGSASPGTSTPESGSASPGSTSESPS | | | |
| | GTAPGSTSESPSGTAPGTSPSGESST | | | |
| | APGSTSESPSGTAPGTSPSGESSTAP | | | |
| | GTSPSGESSTAPGSTSSTAESPGPGT | | | |
| | SPSGESSTAPGTSPSGESSTAPGSTSS | | | |
| | TAESPGPGTSTPESGSASPGTSTPES | | | |
| | GSASPGSTSESPSGTAPGSTSESPSG | | | |
| | TAPGTSTPESGSASPGSTSSTAESPG | | | |
| | PGTSTPESGSASPGSTSESPSGTAPG | | | |
| | TSPSGESSTAPGSTSSTAESPGPGTSP | | | |
| | SGESSTAPGTSTPESGSASPGSTSST | | | |
| | AESPGPGSTSSTAESPGPGSTSSTAE | | | |
| | SPGPGSTSSTAESPGPGTSPSGESST | | | |
| | APGSTSESPSGTAPGSTSESPSGTAP | | | |
| | GTSTPESGPXXXGASASGAPSTXXX | | | |
| | XSESPSGTAPGSTSESPSGTAPGSTS | | | |
| | ESPSGTAPGSTSESPSGTAPGSTSESP | | | |
| | SGTAPGSTSESPSGTAPGTSTPESGS | | | |
| | ASPGTSPSGESSTAPGTSPSGESSTA | | | |
| | PGSTSSTAESPGPGTSPSGESSTAPG | | | |
| | TSTPESGSASPGSTSESPSGTAPGSTS | | | |
| | ESPSGTAPGTSPSGESSTAPGSTSESP | | | |
| | SGTAPGTSTPESGSASPGTSTPESGS | | | |
| | ASPGSTSESPSGTAPGTSTPESGSAS | | | |
| | PGSTSSTAESPGPGSTSESPSGTAPG | | | |
| | STSESPSGTAPGTSPSGESSTAPGSTS | | | |
| | STAESPGPGTSPSGESSTAPGTSTPES | | | |
| | GSASPGTSPSGESSTAPGTSPSGESS | | | |
| | TAPGTSPSGESSTAPGSTSSTAESPG | | | |
| | PGSTSSTAESPGPGTSPSGESSTAPG | | | |
| | SSPSASTGTGPGSSTPSGATGSPGSS | | | |
| | TPSGATGSP | | | |
| AG864 | GASPGTSSTGSPGSSPSASTGTGPGS | 864 | Residue totals: H: 0 E: 0 | 94.91% |
| | SPSASTGTGPGTPGSGTASSSPGSST | | percent: H: 0.0 E: 0.0 | |
| | PSGATGSPGSSPSASTGTGPGASPGT | | | |
| | SSTGSPGTPGSGTASSSPGSSTPSGA | | | |
| | TGSPGTPGSGTASSSPGASPGTSSTG | | | |
| | SPGASPGTSSTGSPGTPGSGTASSSP | | | |
| | GSSTPSGATGSPGASPGTSSTGSPGT | | | |
| | PGSGTASSSPGSSTPSGATGSPGSSP | | | |
| | SASTGTGPGSSPSASTGTGPGSSTPS | | | |
| | GATGSPGSSTPSGATGSPGASPGTSS | | | |
| | TGSPGASPGTSSTGSPGASPGTSSTG | | | |
| | SPGTPGSGTASSSPGASPGTSSTGSP | | | |
| | GASPGTSSTGSPGASPGTSSTGSPGS | | | |
| | SPSASTGTGPGTPGSGTASSSPGASP | | | |
| | GTSSTGSPGASPGTSSTGSPGASPGT | | | |
| | SSTGSPGSSTPSGATGSPGSSTPSGA | | | |
| | TGSPGASPGTSSTGSPGTPGSGTASS | | | |
| | SPGSSTPSGATGSPGSSTPSGATGSP | | | |
| | GSSTPSGATGSPGSSPSASTGTGPGA | | | |
| | SPGTSSTGSPGASPGTSSTGSPGTPG | | | |
| | SGTASSSPGASPGTSSTGSPGASPGT | | | |
| | SSTGSPGASPGTSSTGSPGASPGTSS | | | |
| | TGSPGTPGSGTASSSPGSSTPSGATG | | | |
| | SPGTPGSGTASSSPGSSTPSGATGSP | | | |
| | GTPGSGTASSSPGSSTPSGATGSPGS | | | |
| | STPSGATGSPGSSPSASTGTGPGSSP | | | |
| | SASTGTGPGASPGTSSTGSPGTPGSG | | | |
| | TASSSPGSSTPSGATGSPGSSPSAST | | | |
| | GTGPGSSPSASTGTGPGASPGTSSTG | | | |
| | SPGASPGTSSTGSPGSSTPSGATGSP | | | |
| | GSSPSASTGTGPGASPGTSSTGSPGS | | | |
| | SPSASTGTGPGTPGSGTASSSPGSST | | | |
| | PSGATGSPGSSTPSGATGSPGASPGT | | | |
| | SSTGSP | | | |
| AM875 | GTSTEPSEGSAPGSEPATSGSETPGS | 875 | Residue totals: H: 7 E: 3 | 98.63% |
| | PAGSPTSTEEGSTSSTAESPGPGTST | | percent: H: 0.8 E: 0.3 | |
| | PESGSASPGSTSESPSGTAPGSTSESP | | | |
| | SGTAPGTSTPESGSASPGTSTPESGS | | | |
| | ASPGSEPATSGSETPGTSESATPESG | | | |
| | PGSPAGSPTSTEEGTSTEPSEGSAPG | | | |
| | TSESATPESGPGTSTEPSEGSAPGTS | | | |
| | TEPSEGSAPGSPAGSPTSTEEGTSTE | | | |
| | PSEGSAPGTSTEPSEGSAPGTSESAT | | | |
| | PESGPGTSESATPESGPGTSTEPSEG | | | |
| | SAPGTSTEPSEGSAPGTSESATPESG | | | |
| | PGTSTEPSEGSAPGSEPATSGSETPG | | | |
| | SPAGSPTSTEEGSSTPSGATGSPGTP | | | |
| | GSGTASSSPGSSTPSGATGSPGTSTE | | | |
| | PSEGSAPGTSTEPSEGSAPGSEPATS | | | |
| | GSETPGSPAGSPTSTEEGSPAGSPTS | | | |
| | TEEGTSTEPSEGSAPGASASGAPSTG | | | |
| | GTSESATPESGPGSPAGSPTSTEEGS | | | |
| | PAGSPTSTEEGSTSSTAESPGPGSTS | | | |
| | ESPSGTAPGTSPSGESSTAPGTPGSG | | | |
| | TASSSPGSSTPSGATGSPGSSPSAST | | | |
| | GTGPGSEPATSGSETPGTSESATPES | | | |
| | GPGSEPATSGSETPGSTSSTAESPGP | | | |
| | GSTSSTAESPGPGTSPSGESSTAPGS | | | |
| | EPATSGSETPGSEPATSGSETPGTST | | | |
| | EPSEGSAPGSTSSTAESPGPGTSTPES | | | |
| | GSASPGSTSESPSGTAPGTSTEPSEG | | | |
| | SAPGTSTEPSEGSAPGTSTEPSEGSA | | | |
| | PGSSTPSGATGSPGSSPSASTGTGPG | | | |
| | ASPGTSSTGSPGSEPATSGSETPGTS | | | |
| | ESATPESGPGSPAGSPTSTEEGSSTPS | | | |
| | GATGSPGSSPSASTGTGPGASPGTSS | | | |
| | TGSPGTSESATPESGPGTSTEPSEGS | | | |
| | APGTSTEPSEGSAP | | | |
| AM1318 | GTSTEPSEGSAPGSEPATSGSETPGS | 1318 | Residue totals: H: 7 E: 0 | 99.17% |
| | PAGSPTSTEEGSTSSTAESPGPGTST | | percent: H: 0.7 E: 0.0 | |
| | PESGSASPGSTSESPSGTAPGSTSESP | | | |
| | SGTAPGTSTPESGSASPGTSTPESGS | | | |
| | ASPGSEPATSGSETPGTSESATPESG | | | |
| | PGSPAGSPTSTEEGTSTEPSEGSAPG | | | |
| | TSESATPESGPGTSTEPSEGSAPGTS | | | |
| | TEPSEGSAPGSPAGSPTSTEEGTSTE | | | |
| | PSEGSAPGTSTEPSEGSAPGTSESAT | | | |
| | PESGPGTSESATPESGPGTSTEPSEG | | | |
| | SAPGTSTEPSEGSAPGTSESATPESG | | | |
| | PGTSTEPSEGSAPGSEPATSGSETPG | | | |
| | SPAGSPTSTEEGSSTPSGATGSPGTP | | | |
| | GSGTASSSPGSSTPSGATGSPGTSTE | | | |
| | PSEGSAPGTSTEPSEGSAPGSEPATS | | | |
| | GSETPGSPAGSPTSTEEGSPAGSPTS | | | |
| | TEEGTSTEPSEGSAPGPEPTGPAPSG | | | |
| | GSEPATSGSETPGTSESATPESGPGS | | | |
| | PAGSPTSTEEGTSESATPESGPGSPA | | | |
| | GSPTSTEEGSPAGSPTSTEEGTSESA | | | |
| | TPESGPGSPAGSPTSTEEGSPAGSPT | | | |
| | STEEGSTSSTAESPGPGSTSESPSGT | | | |
| | APGTSPSGESSTAPGSTSESPSGTAP | | | |
| | GSTSESPSGTAPGTSPSGESSTAPGT | | | |
| | STEPSEGSAPGTSESATPESGPGTSE | | | |
| | SATPESGPGSEPATSGSETPGTSESA | | | |
| | TPESGPGTSESATPESGPGTSTEPSE | | | |
| | GSAPGTSESATPESGPGTSTEPSEGS | | | |
| | APGTSPSGESSTAPGTSPSGESSTAP | | | |
| | GTSPSGESSTAPGTSTEPSEGSAPGS | | | |
| | PAGSPTSTEEGTSTEPSEGSAPGSSPS | | | |
| | ASTGTGPGSSTPSGATGSPGSSTPSG | | | |
| | ATGSPGSSTPSGATGSPGSSTPSGAT | | | |
| | GSPGASPGTSSTGSPGASASGAPSTG | | | |
| | GTSPSGESSTAPGSTSSTAESPGPGT | | | |
| | SPSGESSTAPGTSESATPESGPGTST | | | |
| | EPSEGSAPGTSTEPSEGSAPGSSPSA | | | |
| | STGTGPGSSTPSGATGSPGASPGTSS | | | |
| | TGSPGTSTPESGSASPGTSPSGESST | | | |
| | APGTSPSGESSTAPGTSESATPESGP | | | |
| | GSEPATSGSETPGTSTEPSEGSAPGS | | | |
| | TSESPSGTAPGSTSESPSGTAPGTSTP | | | |
| | ESGSASPGSPAGSPTSTEEGTSESAT | | | |
| | PESGPGTSTEPSEGSAPGSPAGSPTS | | | |
| | TEEGTSESATPESGPGSEPATSGSET | | | |
| | PGSSTPSGATGSPGASPGTSSTGSPG | | | |
| | SSTPSGATGSPGSTSESPSGTAPGTS | | | |
| | PSGESSTAPGSTSSTAESPGPGSSTPS | | | |
| | GATGSPGASPGTSSTGSPGTPGSGT | | | |
| | ASSSPGSPAGSPTSTEEGSPAGSPTS | | | |
| | TEEGTSTEPSEGSAP | | | |
| AM923 | MAEPAGSPTSTEEGASPGTSSTGSP | 924 | Residue totals: H: 4 E: 3 | 98.70% |
| | GSSTPSGATGSPGSSTPSGATGSPGT | | percent: H: 0.4 E: 0.3 | |
| | STEPSEGSAPGSEPATSGSETPGSPA | | | |
| | GSPTSTEEGSTSSTAESPGPGTSTPES | | | |
| | GSASPGSTSESPSGTAPGSTSESPSG | | | |
| | TAPGTSTPESGSASPGTSTPESGSAS | | | |
| | PGSEPATSGSETPGTSESATPESGPG | | | |
| | SPAGSPTSTEEGTSTEPSEGSAPGTS | | | |
| | ESATPESGPGTSTEPSEGSAPGTSTE | | | |
| | PSEGSAPGSPAGSPTSTEEGTSTEPS | | | |
| | EGSAPGTSTEPSEGSAPGTSESATPE | | | |
| | SGPGTSESATPESGPGTSTEPSEGSA | | | |
| | PGTSTEPSEGSAPGTSESATPESGPG | | | |
| | TSTEPSEGSAPGSEPATSGSETPGSP | | | |
| | AGSPTSTEEGSSTPSGATGSPGTPGS | | | |
| | GTASSSPGSSTPSGATGSPGTSTEPS | | | |
| | EGSAPGTSTEPSEGSAPGSEPATSGS | | | |
| | ETPGSPAGSPTSTEEGSPAGSPTSTE | | | |
| | EGTSTEPSEGSAPGASASGAPSTGG | | | |
| | TSESATPESGPGSPAGSPTSTEEGSP | | | |
| | AGSPTSTEEGSTSSTAESPGPGSTSE | | | |
| | SPSGTAPGTSPSGESSTAPGTPGSGT | | | |
| | ASSSPGSSTPSGATGSPGSSPSASTG | | | |
| | TGPGSEPATSGSETPGTSESATPESG | | | |
| | PGSEPATSGSETPGSTSSTAESPGPG | | | |
| | STSSTAESPGPGTSPSGESSTAPGSEP | | | |
| | ATSGSETPGSEPATSGSETPGTSTEP | | | |
| | SEGSAPGSTSSTAESPGPGTSTPESG | | | |
| | SASPGSTSESPSGTAPGTSTEPSEGS | | | |
| | APGTSTEPSEGSAPGTSTEPSEGSAP | | | |
| | GSSTPSGATGSPGSSPSASTGTGPGA | | | |
| | SPGTSSTGSPGSEPATSGSETPGTSES | | | |
| | ATPESGPGSPAGSPTSTEEGSSTPSG | | | |
| | ATGSPGSSPSASTGTGPGASPGTSST | | | |
| | GSPGTSESATPESGPGTSTEPSEGSA | | | |
| | PGTSTEPSEGSAP | | | |
| AE912 | MAEPAGSPTSTEEGTPGSGTASSSP | 913 | Residue totals: H: 8 E: 3 | 99.45% |
| | GSSTPSGATGSPGASPGTSSTGSPGS | | percent: H: 0.9 E: 0.3 | |
| | PAGSPTSTEEGTSESATPESGPGTST | | | |
| | EPSEGSAPGSPAGSPTSTEEGTSTEP | | | |
| | SEGSAPGTSTEPSEGSAPGTSESATP | | | |
| | ESGPGSEPATSGSETPGSEPATSGSE | | | |
| | TPGSPAGSPTSTEEGTSESATPESGP | | | |
| | GTSTEPSEGSAPGTSTEPSEGSAPGS | | | |
| | PAGSPTSTEEGTSTEPSEGSAPGTST | | | |
| | EPSEGSAPGTSESATPESGPGTSTEP | | | |
| | SEGSAPGTSESATPESGPGSEPATSG | | | |
| | SETPGTSTEPSEGSAPGTSTEPSEGS | | | |
| | APGTSESATPESGPGTSESATPESGP | | | |
| | GSPAGSPTSTEEGTSESATPESGPGS | | | |
| | EPATSGSETPGTSESATPESGPGTST | | | |
| | EPSEGSAPGTSTEPSEGSAPGTSTEP | | | |
| | SEGSAPGTSTEPSEGSAPGTSTEPSE | | | |
| | GSAPGTSTEPSEGSAPGSPAGSPTST | | | |
| | EEGTSTEPSEGSAPGTSESATPESGP | | | |
| | GSEPATSGSETPGTSESATPESGPGS | | | |
| | EPATSGSETPGTSESATPESGPGTST | | | |
| | EPSEGSAPGTSESATPESGPGSPAGS | | | |
| | PTSTEEGSPAGSPTSTEEGSPAGSPT | | | |
| | STEEGTSESATPESGPGTSTEPSEGS | | | |
| | APGTSESATPESGPGSEPATSGSETP | | | |
| | GTSESATPESGPGSEPATSGSETPGT | | | |
| | SESATPESGPGTSTEPSEGSAPGSPA | | | |
| | GSPTSTEEGTSESATPESGPGSEPAT | | | |
| | SGSETPGTSESATPESGPGSPAGSPT | | | |
| | STEEGSPAGSPTSTEEGTSTEPSEGS | | | |
| | APGTSESATPESGPGTSESATPESGP | | | |
| | GTSESATPESGPGSEPATSGSETPGS | | | |
| | EPATSGSETPGSPAGSPTSTEEGTST | | | |
| | EPSEGSAPGTSTEPSEGSAPGSEPAT | | | |
| | SGSETPGTSESATPESGPGTSTEPSE | | | |
| | GSAP | | | |
| BC 864 | GTSTEPSEPGSAGTSTEPSEPGSAGS | | Residue totals: H: 0 E: 0 | 99.77% |
| | EPATSGTEPSGSGASEPTSTEPGSEP | | percent: H: 0 E: 0 | |
| | ATSGTEPSGSEPATSGTEPSGSEPAT | | | |
| | SGTEPSGSGASEPTSTEPGTSTEPSEP | | | |
| | GSAGSEPATSGTEPSGTSTEPSEPGS | | | |
| | AGSEPATSGTEPSGSEPATSGTEPSG | | | |
| | TSTEPSEPGSAGTSTEPSEPGSAGSE | | | |
| | PATSGTEPSGSEPATSGTEPSGTSEP | | | |
| | STSEPGAGSGASEPTSTEPGTSEPST | | | |
| | SEPGAGSEPATSGTEPSGSEPATSGT | | | |
| | EPSGTSTEPSEPGSAGTSTEPSEPGS | | | |
| | AGSGASEPTSTEPGSEPATSGTEPSG | | | |
| | SEPATSGTEPSGSEPATSGTEPSGSE | | | |
| | PATSGTEPSGTSTEPSEPGSAGSEPA | | | |
| | TSGTEPSGSGASEPTSTEPGTSTEPSE | | | |
| | PGSAGSEPATSGTEPSGSGASEPTST | | | |
| | EPGTSTEPSEPGSAGSGASEPTSTEP | | | |
| | GSEPATSGTEPSGSGASEPTSTEPGS | | | |
| | EPATSGTEPSGSGASEPTSTEPGTST | | | |
| | EPSEPGSAGSEPATSGTEPSGSGASE | | | |
| | PTSTEPGTSTEPSEPGSAGSEPATSG | | | |
| | TEPSGTSTEPSEPGSAGSEPATSGTE | | | |
| | PSGTSTEPSEPGSAGTSTEPSEPGSA | | | |
| | GTSTEPSEPGSAGTSTEPSEPGSAGT | | | |
| | STEPSEPGSAGTSTEPSEPGSAGTSE | | | |
| | PSTSEPGAGSGASEPTSTEPGTSTEP | | | |
| | SEPGSAGTSTEPSEPGSAGTSTEPSE | | | |
| | PGSAGSEPATSGTEPSGSGASEPTST | | | |
| | EPGSEPATSGTEPSGSEPATSGTEPS | | | |
| | GSEPATSGTEPSGSEPATSGTEPSGT | | | |
| | SEPSTSEPGAGSEPATSGTEPSGSGA | | | |
| | SEPTSTEPGTSTEPSEPGSAGSEPATS | | | |
| | GTEPSGSGASEPTSTEPGTSTEPSEP | | | |
| | GSA | | | |

* H: alpha-helix E: beta-sheet

### Example 32

### Analysis of Polypeptide Sequences for Repetitiveness

In this Example, different polypeptides, including several XTEN sequences, were assessed for repetitiveness in the amino acid sequence. Polypeptide amino acid sequences can be assessed for repetitiveness by quantifying the number of times a shorter subsequence appears within the overall polypeptide. For example, a polypeptide of 200 amino acid residues length has a total of 165 overlapping 36-amino acid “blocks” (or “36-mers”) and 198 3-mer “subsequences”, but the number of unique 3-mer subsequences will depend on the amount of repetitiveness within the sequence. For the analyses, different polypeptide sequences were assessed for repetitiveness by determining the subsequence score obtained by application of the following equation:

$$Subsequence score = \frac{{\sum }_{i = 1}^{m} {Count}_{i}}{m} \qquad I$$

wherein: m=(amino acid length of polypeptide)−(amino acid length of subsequence)+1;
and Countᵢ=cumulative number of occurrences of each unique subsequence within sequenceᵢ
   In the analyses of the present Example, the subsequence score for the polypeptides of Table 19 were determined using the foregoing equation in a computer program using the algorithm depicted in FIG. 3, wherein the subsequence length was set at 3 amino acids. The resulting subsequence score is a reflection of the degree of repetitiveness within the polypeptide.

The results, shown in Table 19, indicate that the unstructured polypeptides consisting of 2 or 3 amino acid types have high subsequence scores, while those of consisting of the 12 amino acid motifs of the six amino acids G, S, T, E, P, and A with a low degree of internal repetitiveness, have subsequence scores of less than 10, and in some cases, less than 5. For example, the L288 sequence has two amino acid types and has short, highly repetitive sequences, resulting in a subsequence score of 50.0. The polypeptide J288 has three amino acid types but also has short, repetitive sequences, resulting in a subsequence score of 33.3. Y576 also has three amino acid types, but is not made of internal repeats, reflected in the subsequence score of 15.7 over the first 200 amino acids. W576 consists of four types of amino acids, but has a higher degree of internal repetitiveness, e.g., “GGSG”, resulting in a subsequence score of 23.4. The AD576 consists of four types of 12 amino acid motifs, each consisting of four types of amino acids. Because of the low degree of internal repetitiveness of the individual motifs, the overall subsequence score over the first 200 amino acids is 13.6. In contrast, XTEN's consisting of four motifs contains six types of amino acids, each with a low degree of internal repetitiveness have lower subsequence scores; i.e., AE864 (6.1), AF864 (7.5), and AM875 (4.5), while XTEN consisting of four motifs containing five types of amino acids were intermediate; i.e., AE864, with a score of 7.2.

Conclusions:

The results indicate that the combination of 12 amino acid subsequence motifs, each consisting of four to six amino acid types that are non-repetitive, into a longer XTEN polypeptide results in an overall sequence that is substantially non-repetitive, as indicated by overall average subsequence scores less than 10 and, in many cases, less than 5. This is despite the fact that each subsequence motif may be used multiple times across the sequence. In contrast, polymers created from smaller numbers of amino acid types resulted in higher average subsequence scores, with polypeptides consisting of two amino acid type having higher scores that those consisting of three amino acid types.

TABLE 19
Average subsequence score calculations of polypeptide sequences

| Seq | SEQ ID | | |
|---|---|---|---|
| Name | NO: | Amino Acid Sequence | Score |
| J288 | 783 | GSGGEGGSGGEGGSGGEGGSGGEGGSGGEGGSGGEGGSGGEGGSGGEG | 33.3 |
| | | GSGGEGGSGGEGGSGGEGGSGGEGGSGGEGGSGGEGGSGGEGGSGGEG | |
| | | GSGGEGGSGGEGGSGGEGGSGGEGGSGGEGGSGGEGGSGGEGGSGGEG | |
| | | GSGGEGGSGGEGGSGGEGGSGGEGGSGGEGGSGGEGGSGGEGGSGGEG | |
| | | GSGGEGGSGGEGGSGGEGGSGGEGGSGGEGGSGGEGGSGGEGGSGGEG | |
| | | GSGGEGGSGGEGGSGGEGGSGGEGGSGGEGGSGGEGGSGGEGGSGGEG | |
| K288 | 784 | GEGEGGGEGGEGEGGGEGGEGEGGGEGGEGEGGGEGGEGEGGGEGGEG | 46.9 |
| | | EGGGEGGEGEGGGEGGEGEGGGEGGEGEGGGEGGEGEGGGEGGEGEGG | |
| | | GEGGEGEGGGEGGEGEGGGEGGEGEGGGEGGEGEGGGEGGEGEGGGEG | |
| | | GEGEGGGEGGEGEGGGEGGEGEGGGEGGEGEGGGEGGEGEGGGEGGEG | |
| | | EGGGEGGEGEGGGEGGEGEGGGEGGEGEGGGEGGEGEGGGEGGEGEGG | |
| | | GEGGEGEGGGEGGEGEGGGEGGEGEGGGEGGEGEGGGEGGEGEGGGEG | |
| L288 | 785 | SSESSESSSSESSSESSESSSSESSSESSESSSSESSSESSESSSSESSSESSESSSS | 50.0 |
| | | ESSSESSESSSSESSSESSESSSSESSSESSESSSSESSSESSESSSSESSSESSESS | |
| | | SSESSSESSESSSSESSSESSESSSSESSSESSESSSSESSSESSESSSSESSSESSE | |
| | | SSSSESSSESSESSSSESSSESSESSSSESSSESSESSSSESSSESSESSSSESSSES | |
| | | SESSSSESSSESSESSSSESSSESSESSSSESSSESSESSSSESSSESSESSSSES | |
| Y288 | 786 | GEGSGEGSEGEGSEGSGEGEGSEGSGEGEGGSEGSEGEGGSEGSEGEGGSE | 26.8 |
| | | GSEGEGSGEGSEGEGGSEGSEGEGSGEGSEGEGSEGGSEGEGGSEGSEGEG | |
| | | SGEGSEGEGGEGGSEGEGSEGSGEGEGSGEGSEGEGSEGSGEGEGSGEGSE | |
| | | GEGSEGSGEGEGSEGSGEGEGGSEGSEGEGSEGSGEGEGGEGSGEGEGSG | |
| | | EGSEGEGGGEGSEGEGSGEGGEGEGSEGGSEGEGGSEGGEGEGSEGSGEG | |
| | | EGSEGGSEGEGSEGGSEGEGSEGSGEGEGSEGSGE | |
| Q576 | 787 | GGKPGEGGKPEGGGGKPGGKPEGEGEGKPGGKPEGGGKPGGGEGGKPE | 18.5 |
| | | GGKPEGEGKPGGGEGKPGGKPEGGGGKPEGEGKPGGGGGKPGGKPEGE | |
| | | GKPGGGEGGKPEGKPGEGGEGKPGGKPEGGGEGKPGGGKPGEGGKPGE | |
| | | GKPGGGEGGKPEGGKPEGEGKPGGGEGKPGGKPGEGGKPEGGGEGKPG | |
| | | GKPGEGGEGKPGGGKPEGEGKPGGGKPGGGEGGKPEGEGKPGGKPEGG | |
| | | GEGKPGGKPEGGGKPEGGGEGKPGGGKPGEGGKPGEGEGKPGGKPEGEG | |
| | | KPGGEGGGKPEGKPGGGEGGKPEGGKPGEGGKPEGGKPGEGGEGKPGG | |
| | | GKPGEGGKPEGGGKPEGEGKPGGGGKPGEGGKPEGGKPEGGGEGKPGG | |
| | | GKPEGEGKPGGGEGKPGGKPEGGGGKPGEGGKPEGGKPGGEGGGKPEGE | |
| | | GKPGGKPGEGGGGKPGGKPEGEGKPGEGGEGKPGGKPEGGGEGKPGGKP | |
| | | EGGGEGKPGGGKPGEGGKPEGGGKPGEGGKPGEGGKPEGEGKPGGGEG | |
| | | KPGGKPGEGGKPEGGGEGKPGGKPGGEGGGKPEGGKPGEGGKPEG | |
| U576 | 788 | GEGKPGGKPGSGGGKPGEGGKPGSGEGKPGGKPGSGGSGKPGGKPGEGG | 18.1 |
| | | KPEGGSGGKPGGGGKPGGKPGGEGSGKPGGKPEGGGKPEGGSGGKPGGK | |
| | | PEGGSGGKPGGKPGSGEGGKPGGGKPGGEGKPGSGKPGGEGSGKPGGKP | |
| | | EGGSGGKPGGKPEGGSGGKPGGSGKPGGKPGEGGKPEGGSGGKPGGSGK | |
| | | PGGKPEGGGSGKPGGKPGEGGKPGSGEGGKPGGGKPGGEGKPGSGKPGG | |
| | | EGSGKPGGKPGSGGEGKPGGKPEGGSGGKPGGGKPGGEGKPGSGGKPGE | |
| | | GGKPGSGGGKPGGKPGGEGEGKPGGKPGEGGKPGGEGSGKPGGGGKPG | |
| | | GKPGGEGGKPEGSGKPGGGSGKPGGKPEGGGGKPEGSGKPGGGGKPEGS | |
| | | GKPGGGKPEGGSGGKPGGSGKPGGKPGEGGGKPEGSGKPGGGSGKPGGK | |
| | | PEGGGKPEGGSGGKPGGKPEGGSGGKPGGKPGGEGSGKPGGKPGSGEGG | |
| | | KPGGKPGEGSGGKPGGKPEGGSGGKPGGSGKPGGKPEGGGSGKPGGKPG | |
| | | EGGKPGGEGSGKPGGSGKPG | |
| W576 | 789 | GGSGKPGKPGGSGSGKPGSGKPGGGSGKPGSGKPGGGSGKPGSGKPGGG | 23.4 |
| | | SGKPGSGKPGGGGKPGSGSGKPGGGKPGGSGGKPGGGSGKPGKPGSGGS | |
| | | GKPGSGKPGGGSGGKPGKPGSGGSGGKPGKPGSGGGSGKPGKPGSGGSG | |
| | | GKPGKPGSGGSGGKPGKPGSGGSGKPGSGKPGGGSGKPGSGKPGSGGSG | |
| | | KPGKPGSGGSGKPGSGKPGSGSGKPGSGKPGGGSGKPGSGKPGSGGSGKP | |
| | | GKPGSGGGKPGSGSGKPGGGKPGSGSGKPGGGKPGGSGGKPGGSGGKPG | |
| | | KPGSGGGSGKPGKPGSGGGSGKPGKPGGSGSGKPGSGKPGGGSGKPGSG | |
| | | KPGSGGSGKPGKPGSGGSGGKPGKPGSGGGKPGSGSGKPGGGKPGSGSG | |
| | | KPGGGKPGSGSGKPGGGKPGSGSGKPGGSGKPGSGKPGGGSGGKPGKPG | |
| | | SGGSGKPGSGKPGSGGSGKPGKPGGSGSGKPGSGKPGGGSGKPGSGKPG | |
| | | GGSGKPGSGKPGGGSGKPGSGKPGGGGKPGSGSGKPGGSGGKPGKPGSG | |
| | | GSGGKPGKPGSGGSGKPGSGKPGGGSGGKPGKPGSGG | |
| Y576 | 790 | GEGSGEGSEGEGSEGSGEGEGSEGSGEGEGGSEGSEGEGSEGSGEGEGGE | 15.7 |
| | | GSGEGEGSGEGSEGEGGGEGSEGEGSGEGGEGEGSEGGSEGEGGSEGGEG | |
| | | EGSEGSGEGEGSEGGSEGEGSEGGSEGEGSEGSGEGEGSEGSGEGEGSEGS | |
| | | GEGEGSEGSGEGEGSEGGSEGEGGSEGSEGEGSGEGSEGEGGSEGSEGEG | |
| | | GGEGSEGEGSGEGSEGEGGSEGSEGEGGSEGSEGEGGEGSGEGEGSEGSG | |
| | | EGEGSGEGSEGEGSEGSGEGEGSEGSGEGEGGSEGSEGEGSGEGSEGEGSE | |
| | | GSGEGEGSEGSGEGEGGSEGSEGEGGSEGSEGEGGSEGSEGEGGEGSGEG | |
| | | EGSEGSGEGEGSGEGSEGEGSEGSGEGEGSEGSGEGEGGSEGSEGEGSEGS | |
| | | GEGEGGEGSGEGEGSGEGSEGEGGGEGSEGEGSEGSGEGEGSEGSGEGEG | |
| | | SEGGSEGEGGSEGSEGEGSEGGSEGEGSEGGSEGEGSEGSGEGEGSEGSGE | |
| | | GEGSGEGSEGEGGSEGGEGEGSEGGSEGEGSEGGSEGEGGEGSGEGEGGG | |
| | | EGSEGEGSEGSGEGEGSGEGSE | |
| AE288 | 288 | GTSESATPESGPGSEPATSGSETPGTSESATPESGPGSEPATSGSETPGTSES | 6.0 |
| | | ATPESGPGTSTEPSEGSAPGSPAGSPTSTEEGTSESATPESGPGSEPATSGSE | |
| | | TPGTSESATPESGPGSPAGSPTSTEEGSPAGSPTSTEEGTSTEPSEGSAPGTS | |
| | | ESATPESGPGTSESATPESGPGTSESATPESGPGSEPATSGSETPGSEPATSG | |
| | | SETPGSPAGSPTSTEEGTSTEPSEGSAPGTSTEPSEGSAPGSEPATSGSETPG | |
| | | TSESATPESGPGTSTEPSEGSAP | |
| AG288_1 | 288 | PGASPGTSSTGSPGASPGTSSTGSPGTPGSGTASSSPGSSTPSGATGSPGTPG | 6.9 |
| | | SGTASSSPGSSTPSGATGSPGTPGSGTASSSPGSSTPSGATGSPGSSTPSGAT | |
| | | GSPGSSPSASTGTGPGSSPSASTGTGPGASPGTSSTGSPGTPGSGTASSSPGS | |
| | | STPSGATGSPGSSPSASTGTGPGSSPSASTGTGPGASPGTSSTGSPGASPGTS | |
| | | STGSPGSSTPSGATGSPGSSPSASTGTGPGASPGTSSTGSPGSSPSASTGTGP | |
| | | GTPGSGTASSSPGSSTPSGATGS | |
| AD576 | 791 | GSSESGSSEGGPGSGGEPSESGSSGSSESGSSEGGPGSSESGSSEGGPGSSES | 13.6 |
| | | GSSEGGPGSSESGSSEGGPGSSESGSSEGGPGESPGGSSGSESGSEGSSGPG | |
| | | ESSGSSESGSSEGGPGSSESGSSEGGPGSSESGSSEGGPGSGGEPSESGSSGE | |
| | | SPGGSSGSESGESPGGSSGSESGSGGEPSESGSSGSSESGSSEGGPGSGGEPS | |
| | | ESGSSGSGGEPSESGSSGSEGSSGPGESSGESPGGSSGSESGSGGEPSESGSS | |
| | | GSGGEPSESGSSGSGGEPSESGSSGSSESGSSEGGPGESPGGSSGSESGESPG | |
| | | GSSGSESGESPGGSSGSESGESPGGSSGSESGESPGGSSGSESGSSESGSSEG | |
| | | GPGSGGEPSESGSSGSEGSSGPGESSGSSESGSSEGGPGSGGEPSESGSSGSS | |
| | | ESGSSEGGPGSGGEPSESGSSGESPGGSSGSESGESPGGSSGSESGSSESGSS | |
| | | EGGPGSGGEPSESGSSGSSESGSSEGGPGSGGEPSESGSSGSGGEPSESGSS | |
| | | GESPGGSSGSESGSEGSSGPGESSGSSESGSSEGGPGSEGSSGPGESS | |
| AE576 | 792 | AGSPAGSPTSTEEGTSESATPESGPGTSTEPSEGSAPGSPAGSPTSTEEGTST | 6.1 |
| | | EPSEGSAPGTSTEPSEGSAPGTSESATPESGPGSEPATSGSETPGSEPATSGS | |
| | | ETPGSPAGSPTSTEEGTSESATPESGPGTSTEPSEGSAPGTSTEPSEGSAPGS | |
| | | PAGSPTSTEEGTSTEPSEGSAPGTSTEPSEGSAPGTSESATPESGPGTSTEPSE | |
| | | GSAPGTSESATPESGPGSEPATSGSETPGTSTEPSEGSAPGTSTEPSEGSAPG | |
| | | TSESATPESGPGTSESATPESGPGSPAGSPTSTEEGTSESATPESGPGSEPAT | |
| | | SGSETPGTSESATPESGPGTSTEPSEGSAPGTSTEPSEGSAPGTSTEPSEGSA | |
| | | PGTSTEPSEGSAPGTSTEPSEGSAPGTSTEPSEGSAPGSPAGSPTSTEEGTST | |
| | | EPSEGSAPGTSESATPESGPGSEPATSGSETPGTSESATPESGPGSEPATSGS | |
| | | ETPGTSESATPESGPGTSTEPSEGSAPGTSESATPESGPGSPAGSPTSTEEGS | |
| | | PAGSPTSTEEGSPAGSPTSTEEGTSESATPESGPGTSTEPSEGSAP | |
| AF540 | 793 | GSTSSTAESPGPGSTSSTAESPGPGSTSESPSGTAPGSTSSTAESPGPGSTSST | 8.8 |
| | | AESPGPGTSTPESGSASPGSTSESPSGTAPGTSPSGESSTAPGSTSESPSGTAP | |
| | | GSTSESPSGTAPGTSPSGESSTAPGSTSESPSGTAPGSTSESPSGTAPGTSPSG | |
| | | ESSTAPGSTSESPSGTAPGSTSESPSGTAPGSTSESPSGTAPGTSTPESGSASP | |
| | | GSTSESPSGTAPGTSTPESGSASPGSTSSTAESPGPGSTSSTAESPGPGTSTPE | |
| | | SGSASPGTSTPESGSASPGSTSESPSGTAPGTSTPESGSASPGTSTPESGSASP | |
| | | GSTSESPSGTAPGSTSESPSGTAPGSTSESPSGTAPGSTSSTAESPGPGTSTPE | |
| | | SGSASPGTSTPESGSASPGSTSESPSGTAPGSTSESPSGTAPGTSTPESGSASP | |
| | | GSTSESPSGTAPGSTSESPSGTAPGTSTPESGSASPGTSPSGESSTAPGSTSST | |
| | | AESPGPGTSPSGESSTAPGSTSSTAESPGPGTSTPESGSASPGSTSESPSGTAP | |
| AF504 | 794 | GASPGTSSTGSPGSSPSASTGTGPGSSPSASTGTGPGTPGSGTASSSPGSSTP | 7.0 |
| | | SGATGSPGSNPSASTGTGPGASPGTSSTGSPGTPGSGTASSSPGSSTPSGAT | |
| | | GSPGTPGSGTASSSPGASPGTSSTGSPGASPGTSSTGSPGTPGSGTASSSPGS | |
| | | STPSGATGSPGASPGTSSTGSPGTPGSGTASSSPGSSTPSGATGSPGSNPSAS | |
| | | TGTGPGSSPSASTGTGPGSSTPSGATGSPGSSTPSGATGSPGASPGTSSTGSP | |
| | | GASPGTSSTGSPGASPGTSSTGSPGTPGSGTASSSPGASPGTSSTGSPGASPG | |
| | | TSSTGSPGASPGTSSTGSPGSSPSASTGTGPGTPGSGTASSSPGASPGTSSTG | |
| | | SPGASPGTSSTGSPGASPGTSSTGSPGSSTPSGATGSPGSSTPSGATGSPGAS | |
| | | PGTSSTGSPGTPGSGTASSSPGSSTPSGATGSPGSSTPSGATGSPGSSTPSGA | |
| | | TGSPGSSPSASTGTGPGASPGTSSTGSP | |
| AE864 | 795 | GSPAGSPTSTEEGTSESATPESGPGTSTEPSEGSAPGSPAGSPTSTEEGTSTE | 6.1 |
| | | PSEGSAPGTSTEPSEGSAPGTSESATPESGPGSEPATSGSETPGSEPATSGSE | |
| | | TPGSPAGSPTSTEEGTSESATPESGPGTSTEPSEGSAPGTSTEPSEGSAPGSP | |
| | | AGSPTSTEEGTSTEPSEGSAPGTSTEPSEGSAPGTSESATPESGPGTSTEPSE | |
| | | GSAPGTSESATPESGPGSEPATSGSETPGTSTEPSEGSAPGTSTEPSEGSAPG | |
| | | TSESATPESGPGTSESATPESGPGSPAGSPTSTEEGTSESATPESGPGSEPAT | |
| | | SGSETPGTSESATPESGPGTSTEPSEGSAPGTSTEPSEGSAPGTSTEPSEGSA | |
| | | PGTSTEPSEGSAPGTSTEPSEGSAPGTSTEPSEGSAPGSPAGSPTSTEEGTST | |
| | | EPSEGSAPGTSESATPESGPGSEPATSGSETPGTSESATPESGPGSEPATSGS | |
| | | ETPGTSESATPESGPGTSTEPSEGSAPGTSESATPESGPGSPAGSPTSTEEGS | |
| | | PAGSPTSTEEGSPAGSPTSTEEGTSESATPESGPGTSTEPSEGSAPGTSESAT | |
| | | PESGPGSEPATSGSETPGTSESATPESGPGSEPATSGSETPGTSESATPESGP | |
| | | GTSTEPSEGSAPGSPAGSPTSTEEGTSESATPESGPGSEPATSGSETPGTSES | |
| | | ATPESGPGSPAGSPTSTEEGSPAGSPTSTEEGTSTEPSEGSAPGTSESATPES | |
| | | GPGTSESATPESGPGTSESATPESGPGSEPATSGSETPGSEPATSGSETPGSP | |
| | | AGSPTSTEEGTSTEPSEGSAP | |
| AF864 | 796 | GSTSESPSGTAPGTSPSGESSTAPGSTSESPSGTAPGSTSESPSGTAPGTSTPE | 7.5 |
| | | SGSASPGTSTPESGSASPGSTSESPSGTAPGSTSESPSGTAPGTSPSGESSTAP | |
| | | GSTSESPSGTAPGTSPSGESSTAPGTSPSGESSTAPGSTSSTAESPGPGTSPSG | |
| | | ESSTAPGTSPSGESSTAPGSTSSTAESPGPGTSTPESGSASPGTSTPESGSASP | |
| | | GSTSESPSGTAPGSTSESPSGTAPGTSTPESGSASPGSTSSTAESPGPGTSTPE | |
| | | SGSASPGSTSESPSGTAPGTSPSGESSTAPGSTSSTAESPGPGTSPSGESSTAP | |
| | | GTSTPESGSASPGSTSSTAESPGPGSTSSTAESPGPGSTSSTAESPGPGSTSST | |
| | | AESPGPGTSPSGESSTAPGSTSESPSGTAPGSTSESPSGTAPGTSTPESGPXX | |
| | | XGASASGAPSTXXXXSESPSGTAPGSTSESPSGTAPGSTSESPSGTAPGSTS | |
| | | ESPSGTAPGSTSESPSGTAPGSTSESPSGTAPGTSTPESGSASPGTSPSGESST | |
| | | APGTSPSGESSTAPGSTSSTAESPGPGTSPSGESSTAPGTSTPESGSASPGSTS | |
| | | ESPSGTAPGSTSESPSGTAPGTSPSGESSTAPGSTSESPSGTAPGTSTPESGS | |
| | | ASPGTSTPESGSASPGSTSESPSGTAPGTSTPESGSASPGSTSSTAESPGPGST | |
| | | SESPSGTAPGSTSESPSGTAPGTSPSGESSTAPGSTSSTAESPGPGTSPSGESS | |
| | | TAPGTSTPESGSASPGTSPSGESSTAPGTSPSGESSTAPGTSPSGESSTAPGS | |
| | | TSSTAESPGPGSTSSTAESPGPGTSPSGESSTAPGSSPSASTGTGPGSSTPSG | |
| | | ATGSPGSSTPSGATGSP | |
| AG864 | 864 | GASPGTSSTGSPGSSPSASTGTGPGSSPSASTGTGPGTPGSGTASSSPGSSTP | 7.2 |
| | | SGATGSPGSSPSASTGTGPGASPGTSSTGSPGTPGSGTASSSPGSSTPSGAT | |
| | | GSPGTPGSGTASSSPGASPGTSSTGSPGASPGTSSTGSPGTPGSGTASSSPGS | |
| | | STPSGATGSPGASPGTSSTGSPGTPGSGTASSSPGSSTPSGATGSPGSSPSAS | |
| | | TGTGPGSSPSASTGTGPGSSTPSGATGSPGSSTPSGATGSPGASPGTSSTGSP | |
| | | GASPGTSSTGSPGASPGTSSTGSPGTPGSGTASSSPGASPGTSSTGSPGASPG | |
| | | TSSTGSPGASPGTSSTGSPGSSPSASTGTGPGTPGSGTASSSPGASPGTSSTG | |
| | | SPGASPGTSSTGSPGASPGTSSTGSPGSSTPSGATGSPGSSTPSGATGSPGAS | |
| | | PGTSSTGSPGTPGSGTASSSPGSSTPSGATGSPGSSTPSGATGSPGSSTPSGA | |
| | | TGSPGSSPSASTGTGPGASPGTSSTGSPGASPGTSSTGSPGTPGSGTASSSPG | |
| | | ASPGTSSTGSPGASPGTSSTGSPGASPGTSSTGSPGASPGTSSTGSPGTPGSG | |
| | | TASSSPGSSTPSGATGSPGTPGSGTASSSPGSSTPSGATGSPGTPGSGTASSS | |
| | | PGSSTPSGATGSPGSSTPSGATGSPGSSPSASTGTGPGSSPSASTGTGPGASP | |
| | | GTSSTGSPGTPGSGTASSSPGSSTPSGATGSPGSSPSASTGTGPGSSPSASTG | |
| | | TGPGASPGTSSTGSPGASPGTSSTGSPGSSTPSGATGSPGSSPSASTGTGPG | |
| | | ASPGTSSTGSPGSSPSASTGTGPGTPGSGTASSSPGSSTPSGATGSPGSSTPS | |
| | | GATGSPGASPGTSSTGSP | |
| AG868 | 797 | GGSPGASPGTSSTGSPGSSPSASTGTGPGSSPSASTGTGPGTPGSGTASSSPG | 7.5 |
| | | SSTPSGATGSPGSNPSASTGTGPGASPGTSSTGSPGTPGSGTASSSPGSSTPS | |
| | | GATGSPGTPGSGTASSSPGASPGTSSTGSPGASPGTSSTGSPGTPGSGTASS | |
| | | SPGSSTPSGATGSPGASPGTSSTGSPGTPGSGTASSSPGSSTPSGATGSPGSN | |
| | | PSASTGTGPGSSPSASTGTGPGSSTPSGATGSPGSSTPSGATGSPGASPGTSS | |
| | | TGSPGASPGTSSTGSPGASPGTSSTGSPGTPGSGTASSSPGASPGTSSTGSPG | |
| | | ASPGTSSTGSPGASPGTSSTGSPGSSPSASTGTGPGTPGSGTASSSPGASPGT | |
| | | SSTGSPGASPGTSSTGSPGASPGTSSTGSPGSSTPSGATGSPGSSTPSGATGS | |
| | | PGASPGTSSTGSPGTPGSGTASSSPGSSTPSGATGSPGSSTPSGATGSPGSST | |
| | | PSGATGSPGSSPSASTGTGPGASPGTSSTGSPGASPGTSSTGSPGTPGSGTA | |
| | | SSSPGASPGTSSTGSPGASPGTSSTGSPGASPGTSSTGSPGASPGTSSTGSPG | |
| | | TPGSGTASSSPGSSTPSGATGSPGTPGSGTASSSPGSSTPSGATGSPGTPGSG | |
| | | TASSSPGSSTPSGATGSPGSSTPSGATGSPGSSPSASTGTGPGSSPSASTGTG | |
| | | PGASPGTSSTGSPGTPGSGTASSSPGSSTPSGATGSPGSSPSASTGTGPGSSP | |
| | | SASTGTGPGASPGTSSTGSPGASPGTSSTGSPGSSTPSGATGSPGSSPSASTG | |
| | | TGPGASPGTSSTGSPGSSPSASTGTGPGTPGSGTASSSPGSSTPSGATGSPGS | |
| | | STPSGATGSPGASPGTSSTGSP | |
| AM875 | 798 | GTSTEPSEGSAPGSEPATSGSETPGSPAGSPTSTEEGSTSSTAESPGPGTSTP | 4.5 |
| | | ESGSASPGSTSESPSGTAPGSTSESPSGTAPGTSTPESGSASPGTSTPESGSAS | |
| | | PGSEPATSGSETPGTSESATPESGPGSPAGSPTSTEEGTSTEPSEGSAPGTSE | |
| | | SATPESGPGTSTEPSEGSAPGTSTEPSEGSAPGSPAGSPTSTEEGTSTEPSEG | |
| | | SAPGTSTEPSEGSAPGTSESATPESGPGTSESATPESGPGTSTEPSEGSAPGT | |
| | | STEPSEGSAPGTSESATPESGPGTSTEPSEGSAPGSEPATSGSETPGSPAGSP | |
| | | TSTEEGSSTPSGATGSPGTPGSGTASSSPGSSTPSGATGSPGTSTEPSEGSAP | |
| | | GTSTEPSEGSAPGSEPATSGSETPGSPAGSPTSTEEGSPAGSPTSTEEGTSTE | |
| | | PSEGSAPGASASGAPSTGGTSESATPESGPGSPAGSPTSTEEGSPAGSPTSTE | |
| | | EGSTSSTAESPGPGSTSESPSGTAPGTSPSGESSTAPGTPGSGTASSSPGSSTP | |
| | | SGATGSPGSSPSASTGTGPGSEPATSGSETPGTSESATPESGPGSEPATSGSE | |
| | | TPGSTSSTAESPGPGSTSSTAESPGPGTSPSGESSTAPGSEPATSGSETPGSEP | |
| | | ATSGSETPGTSTEPSEGSAPGSTSSTAESPGPGTSTPESGSASPGSTSESPSGT | |
| | | APGTSTEPSEGSAPGTSTEPSEGSAPGTSTEPSEGSAPGSSTPSGATGSPGSS | |
| | | PSASTGTGPGASPGTSSTGSPGSEPATSGSETPGTSESATPESGPGSPAGSPT | |
| | | STEEGSSTPSGATGSPGSSPSASTGTGPGASPGTSSTGSPGTSESATPESGPG | |
| | | TSTEPSEGSAPGTSTEPSEGSAP | |
| AE912 | 913 | MAEPAGSPTSTEEGTPGSGTASSSPGSSTPSGATGSPGASPGTSSTGSPGSP | 4.5 |
| | | AGSPTSTEEGTSESATPESGPGTSTEPSEGSAPGSPAGSPTSTEEGTSTEPSE | |
| | | GSAPGTSTEPSEGSAPGTSESATPESGPGSEPATSGSETPGSEPATSGSETPG | |
| | | SPAGSPTSTEEGTSESATPESGPGTSTEPSEGSAPGTSTEPSEGSAPGSPAGS | |
| | | PTSTEEGTSTEPSEGSAPGTSTEPSEGSAPGTSESATPESGPGTSTEPSEGSAP | |
| | | GTSESATPESGPGSEPATSGSETPGTSTEPSEGSAPGTSTEPSEGSAPGTSES | |
| | | ATPESGPGTSESATPESGPGSPAGSPTSTEEGTSESATPESGPGSEPATSGSE | |
| | | TPGTSESATPESGPGTSTEPSEGSAPGTSTEPSEGSAPGTSTEPSEGSAPGTS | |
| | | TEPSEGSAPGTSTEPSEGSAPGTSTEPSEGSAPGSPAGSPTSTEEGTSTEPSE | |
| | | GSAPGTSESATPESGPGSEPATSGSETPGTSESATPESGPGSEPATSGSETPG | |
| | | TSESATPESGPGTSTEPSEGSAPGTSESATPESGPGSPAGSPTSTEEGSPAGS | |
| | | PTSTEEGSPAGSPTSTEEGTSESATPESGPGTSTEPSEGSAPGTSESATPESGP | |
| | | GSEPATSGSETPGTSESATPESGPGSEPATSGSETPGTSESATPESGPGTSTE | |
| | | PSEGSAPGSPAGSPTSTEEGTSESATPESGPGSEPATSGSETPGTSESATPES | |
| | | GPGSPAGSPTSTEEGSPAGSPTSTEEGTSTEPSEGSAPGTSESATPESGPGTS | |
| | | ESATPESGPGTSESATPESGPGSEPATSGSETPGSEPATSGSETPGSPAGSPT | |
| | | STEEGTSTEPSEGSAPGTSTEPSEGSAPGSEPATSGSETPGTSESATPESGPG | |
| | | TSTEPSEGSAP | |
| AM923 | 924 | MAEPAGSPTSTEEGASPGTSSTGSPGSSTPSGATGSPGSSTPSGATGSPGTS | 4.5 |
| | | TEPSEGSAPGSEPATSGSETPGSPAGSPTSTEEGSTSSTAESPGPGTSTPESG | |
| | | SASPGSTSESPSGTAPGSTSESPSGTAPGTSTPESGSASPGTSTPESGSASPGS | |
| | | EPATSGSETPGTSESATPESGPGSPAGSPTSTEEGTSTEPSEGSAPGTSESAT | |
| | | PESGPGTSTEPSEGSAPGTSTEPSEGSAPGSPAGSPTSTEEGTSTEPSEGSAP | |
| | | GTSTEPSEGSAPGTSESATPESGPGTSESATPESGPGTSTEPSEGSAPGTSTE | |
| | | PSEGSAPGTSESATPESGPGTSTEPSEGSAPGSEPATSGSETPGSPAGSPTST | |
| | | EEGSSTPSGATGSPGTPGSGTASSSPGSSTPSGATGSPGTSTEPSEGSAPGTS | |
| | | TEPSEGSAPGSEPATSGSETPGSPAGSPTSTEEGSPAGSPTSTEEGTSTEPSE | |
| | | GSAPGASASGAPSTGGTSESATPESGPGSPAGSPTSTEEGSPAGSPTSTEEG | |
| | | STSSTAESPGPGSTSESPSGTAPGTSPSGESSTAPGTPGSGTASSSPGSSTPSG | |
| | | ATGSPGSSPSASTGTGPGSEPATSGSETPGTSESATPESGPGSEPATSGSETP | |
| | | GSTSSTAESPGPGSTSSTAESPGPGTSPSGESSTAPGSEPATSGSETPGSEPA | |
| | | TSGSETPGTSTEPSEGSAPGSTSSTAESPGPGTSTPESGSASPGSTSESPSGTA | |
| | | PGTSTEPSEGSAPGTSTEPSEGSAPGTSTEPSEGSAPGSSTPSGATGSPGSSP | |
| | | SASTGTGPGASPGTSSTGSPGSEPATSGSETPGTSESATPESGPGSPAGSPTS | |
| | | TEEGSSTPSGATGSPGSSPSASTGTGPGASPGTSSTGSPGTSESATPESGPGT | |
| | | STEPSEGSAPGTSTEPSEGSAP | |
| AM1296 | 799 | GTSTEPSEGSAPGSEPATSGSETPGSPAGSPTSTEEGSTSSTAESPGPGTSTP | 4.5 |
| | | ESGSASPGSTSESPSGTAPGSTSESPSGTAPGTSTPESGSASPGTSTPESGSAS | |
| | | PGSEPATSGSETPGTSESATPESGPGSPAGSPTSTEEGTSTEPSEGSAPGTSE | |
| | | SATPESGPGTSTEPSEGSAPGTSTEPSEGSAPGSPAGSPTSTEEGTSTEPSEG | |
| | | SAPGTSTEPSEGSAPGTSESATPESGPGTSESATPESGPGTSTEPSEGSAPGT | |
| | | STEPSEGSAPGTSESATPESGPGTSTEPSEGSAPGSEPATSGSETPGSPAGSP | |
| | | TSTEEGSSTPSGATGSPGTPGSGTASSSPGSSTPSGATGSPGTSTEPSEGSAP | |
| | | GTSTEPSEGSAPGSEPATSGSETPGSPAGSPTSTEEGSPAGSPTSTEEGTSTE | |
| | | PSEGSAPGPEPTGPAPSGGSEPATSGSETPGTSESATPESGPGSPAGSPTSTE | |
| | | EGTSESATPESGPGSPAGSPTSTEEGSPAGSPTSTEEGTSESATPESGPGSPA | |
| | | GSPTSTEEGSPAGSPTSTEEGSTSSTAESPGPGSTSESPSGTAPGTSPSGESST | |
| | | APGSTSESPSGTAPGSTSESPSGTAPGTSPSGESSTAPGTSTEPSEGSAPGTS | |
| | | ESATPESGPGTSESATPESGPGSEPATSGSETPGTSESATPESGPGTSESATP | |
| | | ESGPGTSTEPSEGSAPGTSESATPESGPGTSTEPSEGSAPGTSPSGESSTAPG | |
| | | TSPSGESSTAPGTSPSGESSTAPGTSTEPSEGSAPGSPAGSPTSTEEGTSTEPS | |
| | | EGSAPGSSPSASTGTGPGSSTPSGATGSPGSSTPSGATGSPGSSTPSGATGSP | |
| | | GSSTPSGATGSPGASPGTSSTGSPGASASGAPSTGGTSPSGESSTAPGSTSST | |
| | | AESPGPGTSPSGESSTAPGTSESATPESGPGTSTEPSEGSAPGTSTEPSEGSA | |
| | | PGSSPSASTGTGPGSSTPSGATGSPGASPGTSSTGSPGTSTPESGSASPGTSP | |
| | | SGESSTAPGTSPSGESSTAPGTSESATPESGPGSEPATSGSETPGTSTEPSEG | |
| | | SAPGSTSESPSGTAPGSTSESPSGTAPGTSTPESGSASPGSPAGSPTSTEEGT | |
| | | SESATPESGPGTSTEPSEGSAPGSPAGSPTSTEEGTSESATPESGPGSEPATS | |
| | | GSETPGSSTPSGATGSPGASPGTSSTGSPGSSTPSGATGSPGSTSESPSGTAP | |
| | | GTSPSGESSTAPGSTSSTAESPGPGSSTPSGATGSPGASPGTSSTGSPGTPGS | |
| | | GTASSSPGSPAGSPTSTEEGSPAGSPTSTEEGTSTEPSEGSAP | |

### Example 33

### Calculation of TEPITOPE Scores

TEPITOPE scores of 9 mer peptide sequence can be calculated by adding pocket potentials as described by Sturniolo [Sturniolo, T., et al. (1999) Nat Biotechnol, 17: 555]. In the present Example, separate Tepitope scores were calculated for individual HLA alleles. Table 20 shows as an example the pocket potentials for HLA*0101B, which occurs in high frequency in the Caucasian population. To calculate the TEPITOPE score of a peptide with sequence P1-P2-P3-P4-P5-P6-P7-P8-P9, the corresponding individual pocket potentials in Table 20 were added. The HLA*0101B score of a 9 mer peptide with the sequence FDKLPRTSG is the sum of 0, −1.3, 0, 0.9, 0, −1.8, 0.09, 0, 0.

To evaluate the TEPITOPE scores for long peptides one can repeat the process for all 9 mer subsequences of the sequences. This process can be repeated for the proteins encoded by other HLA alleles. Tables 21-24 give pocket potentials for the protein products of HLA alleles that occur with high frequency in the Caucasian population.

TEPITOPE scores calculated by this method range from approximately −10 to +10. However, 9 mer peptides that lack a hydrophobic amino acid (FKLMVWY) in P1 position have calculated TEPITOPE scores in the range of −1009 to −989. This value is biologically meaningless and reflects the fact that a hydrophobic amino acid serves as an anchor residue for HLA binding and peptides lacking a hydrophobic residue in P1 are considered non binders to HLA. Because most XTEN sequences lack hydrophobic residues, all combinations of 9 mer subsequences will have TEPITOPEs in the range in the range of −1009 to −989. This method confirms that XTEN polypeptides may have few or no predicted T-cell epitopes.

TABLE 20
Pocket potential for HLA*0101B allele.

| Amino | | | | | | | | | |
|---|---|---|---|---|---|---|---|---|---|
| Acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −2.4 | — | −2.7 | −2 | — | −1.9 |
| E | −999 | 0.1 | −1.2 | −0.4 | — | −2.4 | −0.6 | — | −1.9 |
| F | 0 | 0.8 | 0.8 | 0.08 | — | −2.1 | 0.3 | — | −0.4 |
| G | −999 | 0.5 | 0.2 | −0.7 | — | −0.3 | −1.1 | — | −0.8 |
| H | −999 | 0.8 | 0.2 | −0.7 | — | −2.2 | 0.1 | — | −1.1 |
| I | −1 | 1.1 | 1.5 | 0.5 | — | −1.9 | 0.6 | — | 0.7 |
| K | −999 | 1.1 | 0 | −2.1 | — | −2 | −0.2 | — | −1.7 |
| L | −1 | 1 | 1 | 0.9 | — | −2 | 0.3 | — | 0.5 |
| M | −1 | 1.1 | 1.4 | 0.8 | — | −1.8 | 0.09 | — | 0.08 |
| N | −999 | 0.8 | 0.5 | 0.04 | — | −1.1 | 0.1 | — | −1.2 |
| P | −999 | −0.5 | 0.3 | −1.9 | — | −0.2 | 0.07 | — | −1.1 |
| Q | −999 | 1.2 | 0 | 0.1 | — | −1.8 | 0.2 | — | −1.6 |
| R | −999 | 2.2 | 0.7 | −2.1 | — | −1.8 | 0.09 | — | −1 |
| S | −999 | −0.3 | 0.2 | −0.7 | — | −0.6 | −0.2 | — | −0.3 |
| T | −999 | 0 | 0 | −1 | — | −1.2 | 0.09 | — | −0.2 |
| V | −1 | 2.1 | 0.5 | −0.1 | — | −1.1 | 0.7 | — | 0.3 |
| W | 0 | −0.1 | 0 | −1.8 | — | −2.4 | −0.1 | — | −1.4 |
| Y | 0 | 0.9 | 0.8 | −1.1 | — | −2 | 0.5 | — | −0.9 |

TABLE 21
Pocket potential for HLA*0301B allele.

| Amino | | | | | | | | | |
|---|---|---|---|---|---|---|---|---|---|
| acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | 2.3 | — | −2.4 | −0.6 | — | −0.6 |
| E | −999 | 0.1 | −1.2 | −1 | — | −1.4 | −0.2 | — | −0.3 |
| F | −1 | 0.8 | 0.8 | −1 | — | −1.4 | 0.5 | — | 0.9 |
| G | −999 | 0.5 | 0.2 | 0.5 | — | −0.7 | 0.1 | — | 0.4 |
| H | −999 | 0.8 | 0.2 | 0 | — | −0.1 | −0.8 | — | −0.5 |
| I | 0 | 1.1 | 1.5 | 0.5 | — | 0.7 | 0.4 | — | 0.6 |
| K | −999 | 1.1 | 0 | −1 | — | 1.3 | −0.9 | — | −0.2 |
| L | 0 | 1 | 1 | 0 | — | 0.2 | 0.2 | — | −0 |
| M | 0 | 1.1 | 1.4 | 0 | — | −0.9 | 1.1 | — | 1.1 |
| N | −999 | 0.8 | 0.5 | 0.2 | — | −0.6 | −0.1 | — | −0.6 |
| P | −999 | −0.5 | 0.3 | −1 | — | 0.5 | 0.7 | — | −0.3 |
| Q | −999 | 1.2 | 0 | 0 | — | −0.3 | −0.1 | — | −0.2 |
| R | −999 | 2.2 | 0.7 | −1 | — | 1 | −0.9 | — | 0.5 |
| S | −999 | −0.3 | 0.2 | 0.7 | — | −0.1 | 0.07 | — | 1.1 |
| T | −999 | 0 | 0 | −1 | — | 0.8 | −0.1 | — | −0.5 |
| V | 0 | 2.1 | 0.5 | 0 | — | 1.2 | 0.2 | — | 0.3 |
| W | −1 | −0.1 | 0 | −1 | — | −1.4 | −0.6 | — | −1 |
| Y | −1 | 0.9 | 0.8 | −1 | — | −1.4 | −0.1 | — | 0.3 |

TABLE 22
Pocket potential for HLA*0401B allele.

| Amino | | | | | | | | | |
|---|---|---|---|---|---|---|---|---|---|
| acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | 1.4 | — | −1.1 | −0.3 | — | −1.7 |
| E | −999 | 0.1 | −1.2 | 1.5 | — | −2.4 | 0.2 | — | −1.7 |
| F | 0 | 0.8 | 0.8 | −0.9 | — | −1.1 | −1 | — | −1 |
| G | −999 | 0.5 | 0.2 | −1.6 | — | −1.5 | −1.3 | — | −1 |
| H | −999 | 0.8 | 0.2 | 1.1 | — | −1.4 | 0 | — | 0.08 |
| I | −1 | 1.1 | 1.5 | 0.8 | — | −0.1 | 0.08 | — | −0.3 |
| K | −999 | 1.1 | 0 | −1.7 | — | −2.4 | −0.3 | — | −0.3 |
| L | −1 | 1 | 1 | 0.8 | — | −1.1 | 0.7 | — | −1 |
| M | −1 | 1.1 | 1.4 | 0.9 | — | −1.1 | 0.8 | — | −0.4 |
| N | −999 | 0.8 | 0.5 | 0.9 | — | 1.3 | 0.6 | — | −1.4 |
| P | −999 | −0.5 | 0.3 | −1.6 | — | 0 | −0.7 | — | −1.3 |
| Q | −999 | 1.2 | 0 | 0.8 | — | −1.5 | 0 | — | 0.5 |
| R | −999 | 2.2 | 0.7 | −1.9 | — | −2.4 | −1.2 | — | −1 |
| S | −999 | −0.3 | 0.2 | 0.8 | — | 1 | −0.2 | — | 0.7 |
| T | −999 | 0 | 0 | 0.7 | — | 1.9 | −0.1 | — | −1.2 |
| V | −1 | 2.1 | 0.5 | −0.9 | — | 0.9 | 0.08 | — | −0.7 |
| W | 0 | −0.1 | 0 | −1.2 | — | −1 | −1.4 | — | −1 |
| Y | 0 | 0.9 | 0.8 | −1.6 | — | −1.5 | −1.2 | — | −1 |

TABLE 23
Pocket potential for HLA*0701B allele.

| Amino | | | | | | | | | |
|---|---|---|---|---|---|---|---|---|---|
| acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −1.6 | — | −2.5 | −1.3 | — | −1.2 |
| E | −999 | 0.1 | −1.2 | −1.4 | — | −2.5 | 0.9 | — | −0.3 |
| F | 0 | 0.8 | 0.8 | 0.2 | — | −0.8 | 2.1 | — | 2.1 |
| G | −999 | 0.5 | 0.2 | −1.1 | — | −0.6 | 0 | — | −0.6 |
| H | −999 | 0.8 | 0.2 | 0.1 | — | −0.8 | 0.9 | — | −0.2 |
| I | −1 | 1.1 | 1.5 | 1.1 | — | −0.5 | 2.4 | — | 3.4 |
| K | −999 | 1.1 | 0 | −1.3 | — | −1.1 | 0.5 | — | −1.1 |
| L | −1 | 1 | 1 | −0.8 | — | −0.9 | 2.2 | — | 3.4 |
| M | −1 | 1.1 | 1.4 | −0.4 | — | −0.8 | 1.8 | — | 2 |
| N | −999 | 0.8 | 0.5 | −1.1 | — | −0.6 | 1.4 | — | −0.5 |
| P | −999 | −0.5 | 0.3 | −1.2 | — | −0.5 | −0.2 | — | −0.6 |
| Q | −999 | 1.2 | 0 | −1.5 | — | −1.1 | 1.1 | — | −0.9 |
| R | −999 | 2.2 | 0.7 | −1.1 | — | −1.1 | 0.7 | — | −0.8 |
| S | −999 | −0.3 | 0.2 | 1.5 | — | 0.6 | 0.4 | — | −0.3 |
| T | −999 | 0 | 0 | 1.4 | — | −0.1 | 0.9 | — | 0.4 |
| V | −1 | 2.1 | 0.5 | 0.9 | — | 0.1 | 1.6 | — | 2 |
| W | 0 | −0.1 | 0 | −1.1 | — | −0.9 | 1.4 | — | 0.8 |
| Y | 0 | 0.9 | 0.8 | −0.9 | — | −1 | 1.7 | — | 1.1 |

TABLE 24
Pocket potential for HLA*1501B allele.

| Amino | | | | | | | | | |
|---|---|---|---|---|---|---|---|---|---|
| acid | P1 | P2 | P3 | P4 | P5 | P6 | P7 | P8 | P9 |
| A | −999 | 0 | 0 | 0 | — | 0 | 0 | — | 0 |
| C | −999 | 0 | 0 | 0 | — | 0 | 0 | — | 0 |
| D | −999 | −1.3 | −1.3 | −0.4 | — | −0.4 | −0.7 | — | −1.9 |
| E | −999 | 0.1 | −1.2 | −0.6 | — | −1 | −0.7 | — | −1.9 |
| F | −1 | 0.8 | 0.8 | 2.4 | — | −0.3 | 1.4 | — | −0.4 |
| G | −999 | 0.5 | 0.2 | 0 | — | 0.5 | 0 | — | −0.8 |
| H | −999 | 0.8 | 0.2 | 1.1 | — | −0.5 | 0.6 | — | −1.1 |
| I | 0 | 1.1 | 1.5 | 0.6 | — | 0.05 | 1.5 | — | 0.7 |
| K | −999 | 1.1 | 0 | −0.7 | — | −0.3 | −0.3 | — | −1.7 |
| L | 0 | 1 | 1 | 0.5 | — | 0.2 | 1.9 | — | 0.5 |
| M | 0 | 1.1 | 1.4 | 1 | — | 0.1 | 1.7 | — | 0.08 |
| N | −999 | 0.8 | 0.5 | −0.2 | — | 0.7 | 0.7 | — | −1.2 |
| P | −999 | −0.5 | 0.3 | −0.3 | — | −0.2 | 0.3 | — | −1.1 |
| Q | −999 | 1.2 | 0 | −0.8 | — | −0.8 | −0.3 | — | −1.6 |
| R | −999 | 2.2 | 0.7 | 0.2 | — | 1 | −0.5 | — | −1 |
| S | −999 | −0.3 | 0.2 | −0.3 | — | 0.6 | 0.3 | — | −0.3 |
| T | −999 | 0 | 0 | −0.3 | — | −0 | 0.2 | — | −0.2 |
| V | 0 | 2.1 | 0.5 | 0.2 | — | −0.3 | 0.3 | — | 0.3 |
| W | −1 | −0.1 | 0 | 0.4 | — | −0.4 | 0.6 | — | −1.4 |
| Y | −1 | 0.9 | 0.8 | 2.5 | — | 0.4 | 0.7 | — | −0.9 |

### Example 34

### Analysis of FVIII for XTEN Insertion Sites

The selection of XTEN insertion sites within the factor VIII molecule was performed by predicting the locations of permissive sites within loop structures or otherwise flexible surface exposed structural elements. For these analyses, the atomic coordinates of two independently determined X-ray crystallographic structures of FVIII were use (Shen B W, et al. The tertiary structure and domain organization of coagulation factor VIII. Blood. (2008) Feb. 1; 111(3):1240-1247; Ngo J C, et al. Crystal structure of human factor VIII: implications for the formation of the factor IXa-factor VIIIa complex. Structure (2008)16(4):597-606), as well as those of factor VIII and factor VIIIa derived from molecular dynamic simulation (MDS) (Venkateswarlu, D. Structural investigation of zymogenic and activated forms of human blood coagulation factor VIII: a computational molecular dynamics study. BMC Struct Biol. (2010) 10:7). Atomic coodinates in Protein Data Bank (PDB) format were analyzed to identify regions of the FVIII/FVIIIa predicted to have a high degree solvent accessible surface area using the algorithms ASAView (Ahmad S, et al. ASAView: database and tool for solvent accessibility representation in proteins. BMC Bioinformatics (2004) 5:51) and GetArea (Rychkov G, Petukhov M. Joint neighbors approximation of macromolecular solvent accessible surface area. J Comput Chem (2007) 28(12):1974-1989). The resulting set of sites was then further prioritized on the basis of high predicted atomic positional fluctuation based on the basis of the published results of the MDS study. Sites within the acidic peptide regions flanking the A1, A2, and A3 domains, as well as those that appeared by visual inspection to be in areas other than surface exposed loops were deprioritized. The resulting set of potential sites was evaluated on the basis of interspecies sequence conservation, with those sites in regions of high sequence conservation among 20 vertebrate species being ranked more favorably. Additionally, putative clearance receptor binding sites, FVIII interaction sites with other molecules (such as vWF, FIX), domain and exon boundaries were also considered in fusion site selection. Finally, sites within close proximity to mutations implicated in hemophilia A listed in the Haemophilia A Mutation, Search, Test and Resource Site (HAMSTeRS) database were eliminated (Kemball-Cook G, et al. The factor VIII Structure and Mutation Resource Site: HAMSTeRS version 4. Nucleic Acids Res. (1998) 26(1):216-219). Based on these criteria, the construction of 42 FVIII-XTEN variants was proposed (Table 25). Of these, three represent XTEN insertions within the residual B domain sequence, two represent extensions to the C-terminus of the factor VIII molecule, and 37 represent XTEN insertions within structurally defined inter- and intradomain structural elements.

TABLE 25
FVIII XTEN insertion sites and construct designations

| | | Up- | Down- | | | |
|---|---|---|---|---|---|---|
| Con- | | stream | stream | Up- | Down- | |
| struct | Do- | Residue | Residue | stream | stream | XTEN |
| Number | main | No.* | No.* | Sequence | Sequence | Sequence |
| F8X-1 | A1 | 3 | 4 | ATR | RYY | AE42 |
| F8X-2 | A1 | 18 | 19 | YMQ | SDL | AE42 |
| F8X-3 | A1 | 22 | 23 | DLG | ELP | AE42 |
| F8X-4 | A1 | 26 | 27 | LPV | DAR | AE42 |
| F8X-5 | A1 | 40 | 41 | FPF | NTS | AE42 |
| F8X-6 | A1 | 60 | 61 | LFN | IAK | AE42 |
| F8X-7 | A1 | 116 | 117 | YDD | QTS | AE42 |
| F8X-8 | A1 | 130 | 131 | VFP | GGS | AE42 |
| F8X-9 | A1 | 188 | 189 | KEK | TQT | AE42 |
| F8X-10 | A1 | 216 | 217 | NSL | MQD | AE42 |
| F8X-11 | A1 | 230 | 231 | WPK | MHT | AE42 |
| F8X-12 | A1 | 333 | 334 | EEP | QLR | AE42 |
| F8X-13 | A2 | 375 | 376 | SVA | KKH | AE42 |
| F8X-14 | A2 | 403 | 404 | APD | DRS | AE42 |
| F8X-15 | A2 | 442 | 443 | EAI | QHE | AE42 |
| F8X-16 | A2 | 490 | 491 | RRL | PKG | AE42 |
| F8X-17 | A2 | 518 | 519 | TVE | DGP | AE42 |
| F8X-18 | A2 | 599 | 600 | NPA | GVQ | AE42 |
| F8X-19 | A2 | 713 | 714 | CDK | NTG | AE42 |
| F8X-20 | BD | 745 | 746 | SQN | PPV | AE42 |
| F8X-21 | BD | 745 | 746 | SQN | PPV | AE288 |
| F8X-22 | BD** | 745 | 746 | SQN | PPV | AE288 |
| F8X-23 | A3 | 1720 | 1721 | APT | KDE | AE42 |
| F8X-24 | A3 | 1796 | 1797 | EDQ | RQG | AE42 |
| F8X-25 | A3 | 1802 | 1803 | AEP | RKN | AE42 |
| F8X-26 | A3 | 1827 | 1828 | PTK | DEF | AE42 |
| F8X-27 | A3 | 1861 | 1862 | HTN | TLN | AE42 |
| F8X-28 | A3 | 1896 | 1897 | NME | RNC | AE42 |
| F8X-29 | A3 | 1900 | 1901 | NCR | APC | AE42 |
| F8X-30 | A3 | 1904 | 1905 | PCN | IQM | AE42 |
| F8X-31 | A3 | 1937 | 1938 | AQD | QRI | AE42 |
| F8X-32 | C1 | 2019 | 2020 | YSN | KCQ | AE42 |
| F8X-33 | C1 | 2068 | 2069 | EPF | SWI | AE42 |
| F8X-34 | C1 | 2111 | 2112 | GKK | WQT | AE42 |
| F8X-35 | C1 | 2120 | 2121 | NST | GTL | AE42 |
| F8X-36 | C2 | 2171 | 2172 | CDL | NSC | AE42 |
| F8X-37 | C2 | 2188 | 2189 | SDA | QIT | AE42 |
| F8X-38 | C2 | 2227 | 2228 | NPK | EWL | AE42 |
| F8X-39 | C2 | 2277 | 2278 | FQN | GKV | AE42 |
| F8X-40 | CT | 2332 | NA | DLY | NA | AE288 |
| F8X-41 | CT | 2332 | NA | DLY | NA | AG288 |
| F8X-42 | A1 | 3 | 4 | ATR | ATR | AE42 |

*Indicates the amino acid number of the mature FVIII protein
**denotes a construct in which the processing site at R1648 is mutated to alanine to prevent proteolytic processing of FVIII at that location

### Example 35

### Functional Analysis of FVIII-XTEN Constructs

Two FVIII-XTEN fusion proteins, FVIII-AE288 (F8X-40) and FVIII-AG288 (F8X-41), contain an AE288 XTEN or an AE288 XTEN, respectively, fused at the C-terminus of FVIII C2 domain. To determine if FVIII activity was retained after XTEN fusion, HEK293 cells were transfected separately with these two FVIII-XTEN fusion constructs by using polyethylenimine (PEI) in serum-free medium. At 3 or 5 days post-transfection, the cell culture supernatant was tested for FVIII activity by a two-stage chromogenic assay. Purified recombinant FVIII, calibrated against WHO international standard, was used to establish the standard curve in the chromogeinic assay. The fusion protein products of both F8X-40 and F8X-41 constructs were expressed at levels comparable to those of wild-type BDD-FVIII constructs. (Table 26).

TABLE 26
FVIII Titer of FVIII-XTEN fusion proteins in
transient transfection cell culture

| FVIII Molecules | | FVIII 066^{a} | pBC 0114^{a} | FSX-40 | F8X-41 |
|---|---|---|---|---|---|
| FVIII activity | Sample A | 6.42 | 6.68 | 7.47 | 3.32^{b} |
| (IU/ml) | Sample B | 7.13 | 7.61 | 8.25 | Not done |

^{a}Both FVIII 066 and pBC 0114 contain B-domain deleted FVIII without XTEN fusion.
^{b}The F8X-4lsample was from a 3-day transfection while other samples were from a 5-day transient transfection.

### Example 35

### Functional Analysis of FVIII-XTEN Constructs

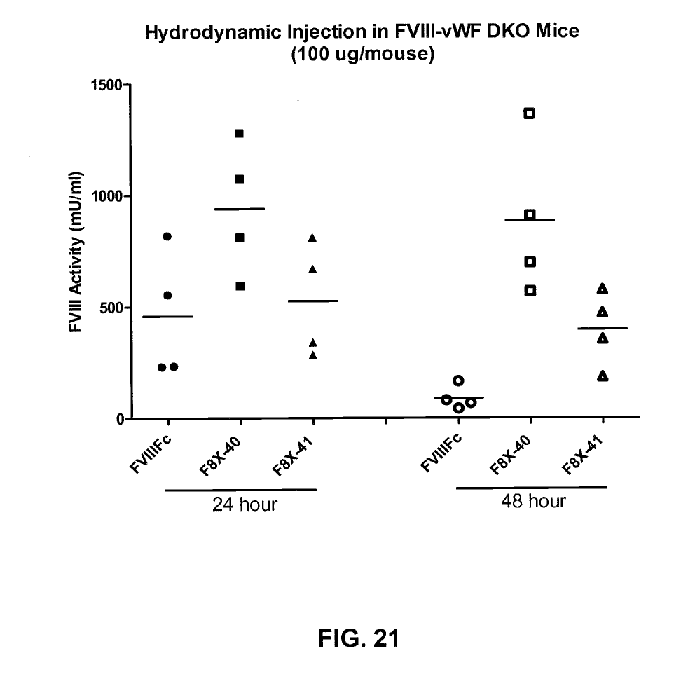

The half-life extension potential of the F8X-40 and F8X-41 constructs was evaluated in FVIII and von Willebrand factor double knock-out mice by hydrodynamic plasmid DNA injection, with a FVIIIFc DNA construct serving as a positive control. Mice were randomly divided into 3 groups with 4 mice per group. Plasmid DNA encoding BDD FVIIIFc fusion protein, F8X-40 or F8X-41, all sharing the same DNA vector backbone, was administered to mice in the respective groups. Approximately 100 micrograms of the appropriate plasmid DNA was injected into each mouse via hydrodynamic injection, and blood plasma samples were collected at 24 hours and 48 hours post-injection. The plasma FVIII activity was measured by a two-stage chromogenic assay using calibrated recombinant FVIII as a standard. As shown in FIG. 21, samples from the F8X-40 and F8X-41 groups showed higher plasma FVIII titers than did those from the BDD FVIIIFc, suggesting FVIII fusion with XTEN prolongs the half-life of FVIII in vivo. Taken together, these data support the conclusion that FVIII-XTEN fusion proteins retained FVIII activity in transient transfection and exhibited prolonged circulating half-life in an animal model.

TABLE 27
Exemplary Biological Activity, Exemplary Assays and Preferred Indications

| Biologically | | Exemplary | |
|---|---|---|---|
| Active protein | Biological Activity | Activity Assays | Preferred Indication: |
| Factor VIII | Coagulation factor VIII is a | Chromogenix assay | Hemophilia A; |
| (Factor VIII; | factor essential for | (Rosen S, Scand J | bleeding; |
| Octocog alfa; | hemostasis. This gene | Haematol (1984) 33 | Factor VIII |
| Moroctocog | encodes coagulation | (Suppl 40):139-45); | deficiency; |
| alfa; | factor VIII, which participates | Chromogenix | bleeding episodes in |
| Recombinant | in the intrinsic pathway of | Coamatic ® Factor | patients with factor |
| Antihemophilic | blood coagulation; factor VIII | VIII assay; one-stage | VIII inhibitor; |
| factor; | is a cofactor for factor IXa | clotting assay | Surgery-related |
| Nordiate; | which, in the presence of | (Lethagen, S., et al., | hemorrhagic |
| ReFacto; | Ca + 2 and phospholipids, | Scandinavian J | episodes |
| Kogenate; | converts factor X to the | Haematology (1986) | |
| Kogenate | activated form Xa. This gene | 37:448-453. | |
| SF; Helixate; | produces two alternatively | One-stage clotting | |
| Recombinate) | spliced transcripts. | assay and two-stage | |
| | Transcript variant I encodes | clotting assay | |
| | a large glycoprotein, isoform | (Barrowcliffe TW, | |
| | a, which circulates in plasma | Semin Thromb | |
| | and associates with von | Hemost. (2002) | |
| | Willebrand | 28(3):247-256); | |
| | factor in a noncovalent | Development of a | |
| | complex. This protein | simple | |
| | undergoes multiple | chromogenic factor | |
| | cleavage events. Transcript | VIII assay for | |
| | variant 2 encodes a putative | clinical use. | |
| | small protein, isoform b, | (Wagenvoord RJ, | |
| | which consists primarily of | Hendrix HH, | |
| | the phospholipid binding | Hemker HC. | |
| | domain of factor VIIIc. This | Haemostasis | |
| | binding domain is essential | 1989; 19(4):196-204) | |
| | for coagulant activity. | | |
| | Defects in this gene results | | |
| | in hemophilia A, a common | | |
| | recessive X-linked | | |
| | coagulation disorder. | | |

TABLE 28
Exemplary CFXTEN comprising FVIII and terminal XTEN

| CFXTEN | |
|---|---|
| Name* | Amino Acid Sequence |
| FVIII- | ATRRYYLGAVELSWDYMQSDLGELPVDARFPPRVPKSFPFNTSVVYKKTLFVEFTDHLFNIAK |
| AE144 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSEPATSGSETPGT |
| | SESATPESGPGSEPATSGSETPGSPAGSPTSTEEGTSTEPSEGSAPGSEPATSGSETPGSEPATSGS |
| | ETPGSEPATSGSETPGTSTEPSEGSAPGTSESATPESGPGSEPATSGSETPGTSTEPSEGSAP |
| FVIII | ATRRYYLGAVELSWDYMQSDLGELPVDARFPPRVPKSFPFNTSVVYKKTLFVEFTVHLFNIAK |
| BDD-2- | PRPPWMGLLGPTIQAEVYDTVVITLKNMASHPVSLHAVGVSYWKASEGAEYDDQTSQREKED |
| AE144 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSEPATSGSETPGT |
| | SESATPESGPGSEPATSGSETPGSPAGSPTSTEEGTSTEPSEGSAPGSEPATSGSETPGSEPATSGS |
| | ETPGSEPATSGSETPGTSTEPSEGSAPGTSESATPESGPGSEPATSGSETPGTSTEPSEGSAP |
| FVIII | ATRRYYLGAVELSWDYMQSDLGELPVDARFPPRVPKSFPFNTSVVYKKTLFVEFTVHLFNIAK |
| BDD-2- | PRPPWMGLLGPTIQAEVYDTVVITLKNMASHPVSLHAVGVSYWKASEGAEYDDQTSQREKED |
| AG144 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGPGSSPSASTGTGP |
| | GSSPSASTGTGPGTPGSGTASSSPGSSTPSGATGSPGSSPSASTGTGPGASPGTSSTGSPGTPGSG |
| | TASSSPGSSTPSGATGSPGTPGSGTASSSPGASPGTSSTGSPGASPGTSSTGSPGTPGSGTASSS |
| FVIII- | ATRRYYLGAVELSWDYMQSDLGELPVDARFPPRVPKSFPFNTSVVYKKTLFVEFTDHLFNIAK |
| AE288 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGTSESATPESGPGS |
| | EPATSGSETPGTSESATPESGPGSEPATSGSETPGTSESATPESGPGTSTEPSEGSAPGSPAGSPTS |
| | TEEGTSESATPESGPGSEPATSGSETPGTSESATPESGPGSPAGSPTSTEEGSPAGSPTSTEEGTST |
| | EPSEGSAPGTSESATPESGPGTSESATPESGPGTSESATPESGPGSEPATSGSETPGSEPATSGSET |
| | PGSPAGSPTSTEEGTSTEPSEGSAPGTSTEPSEGSAPGSEPATSGSETPGTSESATPESGPGTSTEP |
| | SEGSAP |
| FVIII- | ATRRYYLGAVELSWDYMQSDLGELPVDARFPPRVPKSFPFNTSVVYKKTLFVEFTDHLFNIAK |
| AE576 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSPAGSPTSTEEGT |
| | SESATPESGPGTSTEPSEGSAPGSPAGSPTSTEEGTSTEPSEGSAPGTSTEPSEGSAPGTSESATPE |
| | SGPGSEPATSGSETPGSEPATSGSETPGSPAGSPTSTEEGTSESATPESGPGTSTEPSEGSAPGTST |
| | EPSEGSAPGSPAGSPTSTEEGTSTEPSEGSAPGTSTEPSEGSAPGTSESATPESGPGTSTEPSEGSA |
| | PGTSESATPESGPGSEPATSGSETPGTSTEPSEGSAPGTSTEPSEGSAPGTSESATPESGPGTSESA |
| | TPESGPGSPAGSPTSTEEGTSESATPESGPGSEPATSGSETPGTSESATPESGPGTSTEPSEGSAPG |
| | TSTEPSEGSAPGTSTEPSEGSAPGTSTEPSEGSAPGTSTEPSEGSAPGTSTEPSEGSAPGSPAGSPT |
| | STEEGTSTEPSEGSAPGTSESATPESGPGSEPATSGSETPGTSESATPESGPGSEPATSGSETPGTS |
| | ESATPESGPGTSTEPSEGSAPGTSESATPESGPGSPAGSPTSTEEGSPAGSPTSTEEGSPAGSPTST |
| | EEGTSESATPESGPGTSTEPSEGSAP |
| FVIII- | ATRRYYLGAVELSWDYMQSDLGELPVDARFPPRVPKSFPFNTSVVYKKTLFVEFTDHLFNIAK |
| AF576 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSTSSTAESPGPGS |
| | TSSTAESPGPGSTSESPSGTAPGSTSSTAESPGPGSTSSTAESPGPGTSTPESGSASPGSTSESPSGT |
| | APGTSPSGESSTAPGSTSESPSGTAPGSTSESPSGTAPGTSPSGESSTAPGSTSESPSGTAPGSTSES |
| | PSGTAPGTSPSGESSTAPGSTSESPSGTAPGSTSESPSGTAPGSTSESPSGTAPGTSTPESGSASPG |
| | STSESPSGTAPGTSTPESGSASPGSTSSTAESPGPGSTSSTAESPGPGTSTPESGSASPGTSTPESGS |
| | ASPGSTSESPSGTAPGTSTPESGSASPGTSTPESGSASPGSTSESPSGTAPGSTSESPSGTAPGSTSE |
| | SPSGTAPGSTSSTAESPGPGTSTPESGSASPGTSTPESGSASPGSTSESPSGTAPGSTSESPSGTAP |
| | GTSTPESGSASPGSTSESPSGTAPGSTSESPSGTAPGTSTPESGSASPGTSPSGESSTAPGSTSSTAE |
| | SPGPGTSPSGESSTAPGSTSSTAESPGPGTSTPESGSASPGSTSESPSGTAPGSTSSTAESPGPGTST |
| | PESGSASPGTSTPESGSASP |
| FVIII- | ATRRYYLGAVELSWDYMQSDLGELPVDARFPPRVPKSFPFNTSVVYKKTLFVEFTDHLFNIAK |
| AE864 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSPAGSPTSTEEGT |
| | SESATPESGPGTSTEPSEGSAPGSPAGSPTSTEEGTSTEPSEGSAPGTSTEPSEGSAPGTSESATPE |
| | SGPGSEPATSGSETPGSEPATSGSETPGSPAGSPTSTEEGTSESATPESGPGTSTEPSEGSAPGTST |
| | EPSEGSAPGSPAGSPTSTEEGTSTEPSEGSAPGTSTEPSEGSAPGTSESATPESGPGTSTEPSEGSA |
| | PGTSESATPESGPGSEPATSGSETPGTSTEPSEGSAPGTSTEPSEGSAPGTSESATPESGPGTSESA |
| | TPESGPGSPAGSPTSTEEGTSESATPESGPGSEPATSGSETPGTSESATPESGPGTSTEPSEGSAPG |
| | TSTEPSEGSAPGTSTEPSEGSAPGTSTEPSEGSAPGTSTEPSEGSAPGTSTEPSEGSAPGSPAGSPT |
| | STEEGTSTEPSEGSAPGTSESATPESGPGSEPATSGSETPGTSESATPESGPGSEPATSGSETPGTS |
| | ESATPESGPGTSTEPSEGSAPGTSESATPESGPGSPAGSPTSTEEGSPAGSPTSTEEGSPAGSPTST |
| | EEGTSESATPESGPGTSTEPSEGSAPGTSESATPESGPGSEPATSGSETPGTSESATPESGPGSEPA |
| | TSGSETPGTSESATPESGPGTSTEPSEGSAPGSPAGSPTSTEEGTSESATPESGPGSEPATSGSETP |
| | GTSESATPESGPGSPAGSPTSTEEGSPAGSPTSTEEGTSTEPSEGSAPGTSESATPESGPGTSESAT |
| | PESGPGTSESATPESGPGSEPATSGSETPGSEPATSGSETPGSPAGSPTSTEEGTSTEPSEGSAPGT |
| | STEPSEGSAPGSEPATSGSETPGTSESATPESGPGTSTEPSEGSAP |
| FVIII- | ATRRYYLGAVELSWDYMQSDLGELPVDARFPPRVPKSFPFNTSVVYKKTLFVEFTDHLFNIAK |
| AF864 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSTSESPSGTAPGT |
| | SPSGESSTAPGSTSESPSGTAPGSTSESPSGTAPGTSTPESGSASPGTSTPESGSASPGSTSESPSGT |
| | APGSTSESPSGTAPGTSPSGESSTAPGSTSESPSGTAPGTSPSGESSTAPGTSPSGESSTAPGSTSST |
| | AESPGPGTSPSGESSTAPGTSPSGESSTAPGSTSSTAESPGPGTSTPESGSASPGTSTPESGSASPG |
| | STSESPSGTAPGSTSESPSGTAPGTSTPESGSASPGSTSSTAESPGPGTSTPESGSASPGSTSESPSG |
| | TAPGTSPSGESSTAPGSTSSTAESPGPGTSPSGESSTAPGTSTPESGSASPGSTSSTAESPGPGSTSS |
| | TAESPGPGSTSSTAESPGPGSTSSTAESPGPGTSPSGESSTAPGSTSESPSGTAPGSTSESPSGTAP |
| | GTSTPESGPXXXGASASGAPSTXXXXSESPSGTAPGSTSESPSGTAPGSTSESPSGTAPGSTSESP |
| | SGTAPGSTSESPSGTAPGSTSESPSGTAPGTSTPESGSASPGTSPSGESSTAPGTSPSGESSTAPGS |
| | TSSTAESPGPGTSPSGESSTAPGTSTPESGSASPGSTSESPSGTAPGSTSESPSGTAPGTSPSGESST |
| | APGSTSESPSGTAPGTSTPESGSASPGTSTPESGSASPGSTSESPSGTAPGTSTPESGSASPGSTSST |
| | AESPGPGSTSESPSGTAPGSTSESPSGTAPGTSPSGESSTAPGSTSSTAESPGPGTSPSGESSTAPG |
| | TSTPESGSASPGTSPSGESSTAPGTSPSGESSTAPGTSPSGESSTAPGSTSSTAESPGPGSTSSTAES |
| | PGPGTSPSGESSTAPGSSPSASTGTGPGSSTPSGATGSPGSSTPSGATGSP |
| FVIII- | ATRRYYLGAVELSWDYMQSDLGELPVDARFPPRVPKSFPFNTSVVYKKTLFVEFTDHLFNIAK |
| AG864 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGASPGTSSTGSPG |
| | SSPSASTGTGPGSSPSASTGTGPGTPGSGTASSSPGSSTPSGATGSPGSNPSASTGTGPGASPGTS |
| | STGSPGTPGSGTASSSPGSSTPSGATGSPGTPGSGTASSSPGASPGTSSTGSPGASPGTSSTGSPGT |
| | PGSGTASSSPGSSTPSGATGSPGASPGTSSTGSPGTPGSGTASSSPGSSTPSGATGSPGSNPSASTG |
| | TGPGSSPSASTGTGPGSSTPSGATGSPGSSTPSGATGSPGASPGTSSTGSPGASPGTSSTGSPGAS |
| | PGTSSTGSPGTPGSGTASSSPGASPGTSSTGSPGASPGTSSTGSPGASPGTSSTGSPGSSPSASTGT |
| | GPGTPGSGTASSSPGASPGTSSTGSPGASPGTSSTGSPGASPGTSSTGSPGSSTPSGATGSPGSSTP |
| | SGATGSPGASPGTSSTGSPGTPGSGTASSSPGSSTPSGATGSPGSSTPSGATGSPGSSTPSGATGS |
| | PGSSPSASTGTGPGASPGTSSTGSPGASPGTSSTGSPGTPGSGTASSSPGASPGTSSTGSPGASPG |
| | TSSTGSPGASPGTSSTGSPGASPGTSSTGSPGTPGSGTASSSPGSSTPSGATGSPGTPGSGTASSSP |
| | GSSTPSGATGSPGTPGSGTASSSPGSSTPSGATGSPGSSTPSGATGSPGSSPSASTGTGPGSSPSAS |
| | TGTGPGASPGTSSTGSPGTPGSGTASSSPGSSTPSGATGSPGSSPSASTGTGPGSSPSASTGTGPG |
| | ASPGTSSTGSPGASPGTSSTGSPGSSTPSGATGSPGSSPSASTGTGPGASPGTSSTGSPGSSPSAST |
| | GTGPGTPGSGTASSSPGSSTPSGATGSPGSSTPSGATGSPGASPGTSSTGSP |
| FVIII | ATRRYYLGAVELSWDYMQSDLGELPVDARFPPRVPKSFPFNTSVVYKKTLFVEFTVHLFNIAK |
| BDD2- | PRPPWMGLLGPTIQAEVYDTVVITLKNMASHPVSLHAVGVSYWKASEGAEYDDQTSQREKED |
| AE864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSPAGSPTSTEEGT |
| | SESATPESGPGTSTEPSEGSAPGSPAGSPTSTEEGTSTEPSEGSAPGTSTEPSEGSAPGTSESATPE |
| | SGPGSEPATSGSETPGSEPATSGSETPGSPAGSPTSTEEGTSESATPESGPGTSTEPSEGSAPGTST |
| | EPSEGSAPGSPAGSPTSTEEGTSTEPSEGSAPGTSTEPSEGSAPGTSESATPESGPGTSTEPSEGSA |
| | PGTSESATPESGPGSEPATSGSETPGTSTEPSEGSAPGTSTEPSEGSAPGTSESATPESGPGTSESA |
| | TPESGPGSPAGSPTSTEEGTSESATPESGPGSEPATSGSETPGTSESATPESGPGTSTEPSEGSAPG |
| | TSTEPSEGSAPGTSTEPSEGSAPGTSTEPSEGSAPGTSTEPSEGSAPGTSTEPSEGSAPGSPAGSPT |
| | STEEGTSTEPSEGSAPGTSESATPESGPGSEPATSGSETPGTSESATPESGPGSEPATSGSETPGTS |
| | ESATPESGPGTSTEPSEGSAPGTSESATPESGPGSPAGSPTSTEEGSPAGSPTSTEEGSPAGSPTST |
| | EEGTSESATPESGPGTSTEPSEGSAPGTSESATPESGPGSEPATSGSETPGTSESATPESGPGSEPA |
| | TSGSETPGTSESATPESGPGTSTEPSEGSAPGSPAGSPTSTEEGTSESATPESGPGSEPATSGSETP |
| | GTSESATPESGPGSPAGSPTSTEEGSPAGSPTSTEEGTSTEPSEGSAPGTSESATPESGPGTSESAT |
| | PESGPGTSESATPESGPGSEPATSGSETPGSEPATSGSETPGSPAGSPTSTEEGTSTEPSEGSAPGT |
| | STEPSEGSAPGSEPATSGSETPGTSESATPESGPGTSTEPSEGSAP |
| FVIII | ATRRYYLGAVELSWDYMQSDLGELPVDARFPPRVPKSFPFNTSVVYKKTLFVEFTVHLFNIAK |
| BDD2- | PRPPWMGLLGPTIQAEVYDTVVITLKNMASHPVSLHAVGVSYWKASEGAEYDDQTSQREKED |
| AG864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGASPGTSSTGSPG |
| | SSPSASTGTGPGSSPSASTGTGPGTPGSGTASSSPGSSTPSGATGSPGSNPSASTGTGPGASPGTS |
| | STGSPGTPGSGTASSSPGSSTPSGATGSPGTPGSGTASSSPGASPGTSSTGSPGASPGTSSTGSPGT |
| | PGSGTASSSPGSSTPSGATGSPGASPGTSSTGSPGTPGSGTASSSPGSSTPSGATGSPGSNPSASTG |
| | TGPGSSPSASTGTGPGSSTPSGATGSPGSSTPSGATGSPGASPGTSSTGSPGASPGTSSTGSPGAS |
| | PGTSSTGSPGTPGSGTASSSPGASPGTSSTGSPGASPGTSSTGSPGASPGTSSTGSPGSSPSASTGT |
| | GPGTPGSGTASSSPGASPGTSSTGSPGASPGTSSTGSPGASPGTSSTGSPGSSTPSGATGSPGSSTP |
| | SGATGSPGASPGTSSTGSPGTPGSGTASSSPGSSTPSGATGSPGSSTPSGATGSPGSSTPSGATGS |
| | PGSSPSASTGTGPGASPGTSSTGSPGASPGTSSTGSPGTPGSGTASSSPGASPGTSSTGSPGASPG |
| | TSSTGSPGASPGTSSTGSPGASPGTSSTGSPGTPGSGTASSSPGSSTPSGATGSPGTPGSGTASSSP |
| | GSSTPSGATGSPGTPGSGTASSSPGSSTPSGATGSPGSSTPSGATGSPGSSPSASTGTGPGSSPSAS |
| | TGTGPGASPGTSSTGSPGTPGSGTASSSPGSSTPSGATGSPGSSPSASTGTGPGSSPSASTGTGPG |
| | ASPGTSSTGSPGASPGTSSTGSPGSSTPSGATGSPGSSPSASTGTGPGASPGTSSTGSPGSSPSAST |
| | GTGPGTPGSGTASSSPGSSTPSGATGSPGSSTPSGATGSPGASPGTSSTGSP |
| FVIII- | ATRRYYLGAVELSWDYMQSDLGELPVDARFPPRVPKSFPFNTSVVYKKTLFVEFTDHLFNIAK |
| AM875 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGTSTEPSEGSAPGS |
| | EPATSGSETPGSPAGSPTSTEEGSTSSTAESPGPGTSTPESGSASPGSTSESPSGTAPGSTSESPSGT |
| | APGTSTPESGSASPGTSTPESGSASPGSEPATSGSETPGTSESATPESGPGSPAGSPTSTEEGTSTE |
| | PSEGSAPGTSESATPESGPGTSTEPSEGSAPGTSTEPSEGSAPGSPAGSPTSTEEGTSTEPSEGSAP |
| | GTSTEPSEGSAPGTSESATPESGPGTSESATPESGPGTSTEPSEGSAPGTSTEPSEGSAPGTSESAT |
| | PESGPGTSTEPSEGSAPGSEPATSGSETPGSPAGSPTSTEEGSSTPSGATGSPGTPGSGTASSSPGS |
| | STPSGATGSPGTSTEPSEGSAPGTSTEPSEGSAPGSEPATSGSETPGSPAGSPTSTEEGSPAGSPTS |
| | TEEGTSTEPSEGSAPGASASGAPSTGGTSESATPESGPGSPAGSPTSTEEGSPAGSPTSTEEGSTSS |
| | TAESPGPGSTSESPSGTAPGTSPSGESSTAPGTPGSGTASSSPGSSTPSGATGSPGSSPSASTGTGP |
| | GSEPATSGSETPGTSESATPESGPGSEPATSGSETPGSTSSTAESPGPGSTSSTAESPGPGTSPSGE |
| | SSTAPGSEPATSGSETPGSEPATSGSETPGTSTEPSEGSAPGSTSSTAESPGPGTSTPESGSASPGS |
| | TSESPSGTAPGTSTEPSEGSAPGTSTEPSEGSAPGTSTEPSEGSAPGSSTPSGATGSPGSSPSASTG |
| | TGPGASPGTSSTGSPGSEPATSGSETPGTSESATPESGPGSPAGSPTSTEEGSSTPSGATGSPGSSP |
| | SASTGTGPGASPGTSSTGSPGTSESATPESGPGTSTEPSEGSAPGTSTEPSEGSAP |
| FVIII- | ATRRYYLGAVELSWDYMQSDLGELPVDARFPPRVPKSFPFNTSVVYKKTLFVEFTDHLFNIAK |
| AM1318 | PRPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGTSTEPSEGSAPGS |
| | EPATSGSETPGSPAGSPTSTEEGSTSSTAESPGPGTSTPESGSASPGSTSESPSGTAPGSTSESPSGT |
| | APGTSTPESGSASPGTSTPESGSASPGSEPATSGSETPGTSESATPESGPGSPAGSPTSTEEGTSTE |
| | PSEGSAPGTSESATPESGPGTSTEPSEGSAPGTSTEPSEGSAPGSPAGSPTSTEEGTSTEPSEGSAP |
| | GTSTEPSEGSAPGTSESATPESGPGTSESATPESGPGTSTEPSEGSAPGTSTEPSEGSAPGTSESAT |
| | PESGPGTSTEPSEGSAPGSEPATSGSETPGSPAGSPTSTEEGSSTPSGATGSPGTPGSGTASSSPGS |
| | STPSGATGSPGTSTEPSEGSAPGTSTEPSEGSAPGSEPATSGSETPGSPAGSPTSTEEGSPAGSPTS |
| | TEEGTSTEPSEGSAPGPEPTGPAPSGGSEPATSGSETPGTSESATPESGPGSPAGSPTSTEEGTSES |
| | ATPESGPGSPAGSPTSTEEGSPAGSPTSTEEGTSESATPESGPGSPAGSPTSTEEGSPAGSPTSTEE |
| | GSTSSTAESPGPGSTSESPSGTAPGTSPSGESSTAPGSTSESPSGTAPGSTSESPSGTAPGTSPSGES |
| | STAPGTSTEPSEGSAPGTSESATPESGPGTSESATPESGPGSEPATSGSETPGTSESATPESGPGTS |
| | ESATPESGPGTSTEPSEGSAPGTSESATPESGPGTSTEPSEGSAPGTSPSGESSTAPGTSPSGESST |
| | APGTSPSGESSTAPGTSTEPSEGSAPGSPAGSPTSTEEGTSTEPSEGSAPGSSPSASTGTGPGSSTP |
| | SGATGSPGSSTPSGATGSPGSSTPSGATGSPGSSTPSGATGSPGASPGTSSTGSPGASASGAPSTG |
| | GTSPSGESSTAPGSTSSTAESPGPGTSPSGESSTAPGTSESATPESGPGTSTEPSEGSAPGTSTEPSE |
| | GSAPGSSPSASTGTGPGSSTPSGATGSPGASPGTSSTGSPGTSTPESGSASPGTSPSGESSTAPGTS |
| | PSGESSTAPGTSESATPESGPGSEPATSGSETPGTSTEPSEGSAPGSTSESPSGTAPGSTSESPSGT |
| | APGTSTPESGSASPGSPAGSPTSTEEGTSESATPESGPGTSTEPSEGSAPGSPAGSPTSTEEGTSES |
| | ATPESGPGSEPATSGSETPGSSTPSGATGSPGASPGTSSTGSPGSSTPSGATGSPGSTSESPSGTAP |
| | GTSPSGESSTAPGSTSSTAESPGPGSSTPSGATGSPGASPGTSSTGSPGTPGSGTASSSPGSPAGSP |
| | TSTEEGSPAGSPTSTEEGTSTEPSEGSAP |
| AE144- | GSEPATSGSETPGTSESATPESGPGSEPATSGSETPGSPAGSPTSTEEGTSTEPSEGSAPGSEPATS |
| FVIII | GSETPGSEPATSGSETPGSEPATSGSETPGTSTEPSEGSAPGTSESATPESGPGSEPATSGSETPGT |
| | STEPSEGSAPGATRRYYLGAVELSWDYMQSDLGELPVDARFPPRVPKSFPFNTSVVYKKTLFV |
| | EFTDHLFNIAKPRPPWMGLLGPTIQAEVYDTVVITLKNMASHPVSLHAVGVSYWKASEGAEY |
| | DDQTSQREKEDDKVFPGGSHTYVWQVLKENGPMASDPLCLTYSYLSHVDLVKDLNSGLIGAL |
| | LVCREGSLAKEKTQTLHKFILLFAVFDEGKSWHSETKNSLMQDRDAASARAWPKMHTVNGY |
| | VNRSLPGLIGCHRKSVYWHVIGMGTTPEVHSIFLEGHTFLVRNHRQASLEISPITFLTAQTLLMD |
| | LGQFLLFCHISSHQHDGMEAYVKVDSCPEEPQLRMKNNEEAEDYDDDLTDSEMDVVRFDDD |
| | NSPSFIQIRSVAKKHPKTWVHYIAAEEEDWDYAPLVLAPDDRSYKSQYLNNGPQRIGRKYKKV |
| | RFMAYTDETFKTREAIQHESGILGPLLYGEVGDTLLIIFKNQASRPYNIYPHGITDVRPLYSRRLP |
| | KGVKHLKDFPILPGEIFKYKWTVTVEDGPTKSDPRCLTRYYSSFVNMERDLASGLIGPLLICYK |
| | ESVDQRGNQIMSDKRNVILFSVFDENRSWYLTENIQRFLPNPAGVQLEDPEFQASNIMHSINGY |
| | VFDSLQLSVCLHEVAYWYILSIGAQTDFLSVFFSGYTFKHKMVYEDTLTLFPFSGETVFMSME |
| | NPGLWILGCHNSDFRNRGMTALLKVSSCDKNTGDYYEDSYEDISAYLLSKNNAIEPRSFSQNS |
| | RHPSTRQKQFNATTIPENDIEKTDPWFAHRTPMPKIQNVSSSDLLMLLRQSPTPHGLSLSDLQE |
| | AKYETFSDDPSPGAIDSNNSLSEMTHFRPQLHHSGDMVFTPESGLQLRLNEKLGTTAATELKKL |
| | DFKVSSTSNNLISTIPSDNLAAGTDNTSSLGPPSMPVHYDSQLDTTLFGKKSSPLTESGGPLSLSE |
| | ENNDSKLLESGLMNSQESSWGKNVSSTESGRLFKGKRAHGPALLTKDNALFKVSISLLKTNKT |
| | SNNSATNRKTHIDGPSLLIENSPSVWQNILESDTEFKKVTPLIHDRMLMDKNATALRLNHMSN |
| | KTTSSKNMEMVQQKKEGPIPPDAQNPDMSFFKMLFLPESARWIQRTHGKNSLNSGQGPSPKQL |
| | VSLGPEKSVEGQNFLSEKNKVVVGKGEFTKDVGLKEMVFPSSRNLFLTNLDNLHENNTHNQE |
| | KKIQEEIEKKETLIQENVVLPQIHTVTGTKNFMKNLFLLSTRQNVEGSYDGAYAPVLQDFRSLN |
| | DSTNRTKKHTAHFSKKGEEENLEGLGNQTKQIVEKYACTTRISPNTSQQNFVTQRSKRALKQF |
| | RLPLEETELEKRIIVDDTSTQWSKNMKHLTPSTLTQIDYNEKEKGAITQSPLSDCLTRSHSIPQA |
| | NRSPLPIAKVSSFPSIRPIYLTRVLFQDNSSHLPAASYRKKDSGVQESSHFLQGAKKNNLSLAILT |
| | LEMTGDQREVGSLGTSATNSVTYKKVENTVLPKPDLPKTSGKVELLPKVHIYQKDLFPTETSN |
| | GSPGHLDLVEGSLLQGTEGAIKWNEANRPGKVPFLRVATESSAKTPSKLLDPLAWDNHYGTQI |
| | PKEEWKSQEKSPEKTAFKKKDTILSLNACESNHAIAAINEGQNKPEIEVTWAKQGRTERLCSQN |
| | PPVLKRHQREITRTTLQSDQEEIDYDDTISVEMKKEDFDIYDEDENQSPRSFQKKTRHYFIAAVE |
| | RLWDYGMSSSPHVLRNRAQSGSVPQFKKVVFQEFTDGSFTQPLYRGELNEHLGLLGPYIRAEV |
| | EDNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFTE |
| | NMERNCRAPCNIQMEDPTFKENYRFHAINGYIMDTLPGLVMAQDQRIRWYLLSMGSNENIHSI |
| | HFSGHVFTVRKKEEYKMALYNLYPGVFETVEMLPSKAGIWRVECLIGEHLHAGMSTLFLVYS |
| | NKCQTPLGMASGHIRDFQITASGQYGQWAPKLARLHYSGSINAWSTKEPFSWIKVDLLAPMII |
| | HGIKTQGARQKFSSLYISQFIIMYSLDGKKWQTYRGNSTGTLMVFFGNVDSSGIKHNIFNPPIIA |
| | RYIRLHPTHYSIRSTLRMELMGCDLNSCSMPLGMESKAISDAQITASSYFTNMFATWSPSKARL |
| | HLQGRSNAWRPQVNNPKEWLQVDFQKTMKVTGVTTQGVKSLLTSMYVKEFLISSSQDGHQW |
| | TLFFQNGKVKVFQGNQDSFTPVVNSLDPPLLTRYLRIHPQSWVHQIALRMEVLGCEAQDLY |
| AE288- | GTSESATPESGPGSEPATSGSETPGTSESATPESGPGSEPATSGSETPGTSESATPESGPGTSTEPS |
| FVIII | EGSAPGSPAGSPTSTEEGTSESATPESGPGSEPATSGSETPGTSESATPESGPGSPAGSPTSTEEGS |
| | PAGSPTSTEEGTSTEPSEGSAPGTSESATPESGPGTSESATPESGPGTSESATPESGPGSEPATSGS |
| | ETPGSEPATSGSETPGSPAGSPTSTEEGTSTEPSEGSAPGTSTEPSEGSAPGSEPATSGSETPGTSE |
| | SATPESGPGTSTEPSEGSAPGATRRYYLGAVELSWDYMQSDLGELPVDARFPPRVPKSFPFNTS |
| | VVYKKTLFVEFTDHLFNIAKPRPPWMGLLGPTIQAEVYDTVVITLKNMASHPVSLHAVGVSY |
| | WKASEGAEYDDQTSQREKEDDKVFPGGSHTYVWQVLKENGPMASDPLCLTYSYLSHVDLVK |
| | DLNSGLIGALLVCREGSLAKEKTQTLHKFILLFAVFDEGKSWHSETKNSLMQDRDAASARAWP |
| | KMHTVNGYVNRSLPGLIGCHRKSVYWHVIGMGTTPEVHSIFLEGHTFLVRNHRQASLEISPITF |
| | LTAQTLLMDLGQFLLFCHISSHQHDGMEAYVKVDSCPEEPQLRMKNNEEAEDYDDDLTDSEM |
| | DVVRFDDDNSPSFIQIRSVAKKHPKTWVHYIAAEEEDWDYAPLVLAPDDRSYKSQYLNNGPQ |
| | RIGRKYKKVRFMAYTDETFKTREAIQHESGILGPLLYGEVGDTLLIIFKNQASRPYNIYPHGITD |
| | VRPLYSRRLPKGVKHLKDFPILPGEIFKYKWTVTVEDGPTKSDPRCLTRYYSSFVNMERDLAS |
| | GLIGPLLICYKESVDQRGNQIMSDKRNVILFSVFDENRSWYLTENIQRFLPNPAGVQLEDPEFQA |
| | SNIMHSINGYVFDSLQLSVCLHEVAYWYILSIGAQTDFLSVFFSGYTFKHKMVYEDTLTLFPFS |
| | GETVFMSMENPGLWILGCHNSDFRNRGMTALLKVSSCDKNTGDYYEDSYEDISAYLLSKNNA |
| | IEPRSFSQNSRHPSTRQKQFNATTIPENDIEKTDPWFAHRTPMPKIQNVSSSDLLMLLRQSPTPH |
| | GLSLSDLQEAKYETFSDDPSPGAIDSNNSLSEMTHFRPQLHHSGDMVFTPESGLQLRLNEKLGT |
| | TAATELKKLDFKVSSTSNNLISTIPSDNLAAGTDNTSSLGPPSMPVHYDSQLDTTLFGKKSSPLT |
| | ESGGPLSLSEENNDSKLLESGLMNSQESSWGKNVSSTESGRLFKGKRAHGPALLTKDNALFKV |
| | SISLLKTNKTSNNSATNRKTHIDGPSLLIENSPSVWQNILESDTEFKKVTPLIHDRMLMDKNATA |
| | LRLNHMSNKTTSSKNMEMVQQKKEGPIPPDAQNPDMSFFKMLFLPESARWIQRTHGKNSLNS |
| | GQGPSPKQLVSLGPEKSVEGQNFLSEKNKVVVGKGEFTKDVGLKEMVFPSSRNLFLTNLDNLH |
| | ENNTHNQEKKIQEEIEKKETLIQENVVLPQIHTVTGTKNFMKNLFLLSTRQNVEGSYDGAYAPV |
| | LQDFRSLNDSTNRTKKHTAHFSKKGEEENLEGLGNQTKQIVEKYACTTRISPNTSQQNFVTQRS |
| | KRALKQFRLPLEETELEKRIIVDDTSTQWSKNMKHLTPSTLTQIDYNEKEKGAITQSPLSDCLTR |
| | SHSIPQANRSPLPIAKVSSFPSIRPIYLTRVLFQDNSSHLPAASYRKKDSGVQESSHFLQGAKKNN |
| | LSLAILTLEMTGDQREVGSLGTSATNSVTYKKVENTVLPKPDLPKTSGKVELLPKVHIYQKDLF |
| | PTETSNGSPGHLDLVEGSLLQGTEGAIKWNEANRPGKVPFLRVATESSAKTPSKLLDPLAWDN |
| | HYGTQIPKEEWKSQEKSPEKTAFKKKDTILSLNACESNHAIAAINEGQNKPEIEVTWAKQGRTE |
| | RLCSQNPPVLKRHQREITRTTLQSDQEEIDYDDTISVEMKKEDFDIYDEDENQSPRSFQKKTRH |
| | YFIAAVERLWDYGMSSSPHVLRNRAQSGSVPQFKKVVFQEFTDGSFTQPLYRGELNEHLGLLG |
| | PYIRAEVEDNIMVTFRNQASRPYSFYSSLISYEEDQRQGAEPRKNFVKPNETKTYFWKVQHHM |
| | APTKDEFDCKAWAYFSDVDLEKDVHSGLIGPLLVCHTNTLNPAHGRQVTVQEFALFFTIFDET |
| | KSWYFTENMERNCRAPCNIQMEDPTFKENYRFHAINGYIMDTLPGLVMAQDQRIRWYLLSMG |
| | SNENIHSIHFSGHVFTVRKKEEYKMALYNLYPGVFETVEMLPSKAGIWRVECLIGEHLHAGMS |
| | TLFLVYSNKCQTPLGMASGHIRDFQITASGQYGQWAPKLARLHYSGSINAWSTKEPFSWIKVD |
| | LLAPMIIHGIKTQGARQKFSSLYISQFIIMYSLDGKKWQTYRGNSTGTLMVFFGNVDSSGIKHNI |
| | FNPPIIARYIRLHPTHYSIRSTLRMELMGCDLNSCSMPLGMESKAISDAQITASSYFTNMFATWS |
| | PSKARLHLQGRSNAWRPQVNNPKEWLQVDFQKTMKVTGVTTQGVKSLLTSMYVKEFLISSSQ |
| | DGHQWTLFFQNGKVKVFQGNQDSFTPVVNSLDPPLLTRYLRIHPQSWVHQIALRMEVLGCEA |
| | QDLY |
| AE576- | GSPAGSPTSTEEGTSESATPESGPGTSTEPSEGSAPGSPAGSPTSTEEGTSTEPSEGSAPGTSTEPS |
| FVIII | EGSAPGTSESATPESGPGSEPATSGSETPGSEPATSGSETPGSPAGSPTSTEEGTSESATPESGPGT |
| | STEPSEGSAPGTSTEPSEGSAPGSPAGSPTSTEEGTSTEPSEGSAPGTSTEPSEGSAPGTSESATPE |
| | SGPGTSTEPSEGSAPGTSESATPESGPGSEPATSGSETPGTSTEPSEGSAPGTSTEPSEGSAPGTSE |
| | SATPESGPGTSESATPESGPGSPAGSPTSTEEGTSESATPESGPGSEPATSGSETPGTSESATPESG |
| | PGTSTEPSEGSAPGTSTEPSEGSAPGTSTEPSEGSAPGTSTEPSEGSAPGTSTEPSEGSAPGTSTEP |
| | SEGSAPGSPAGSPTSTEEGTSTEPSEGSAPGTSESATPESGPGSEPATSGSETPGTSESATPESGPG |
| | SEPATSGSETPGTSESATPESGPGTSTEPSEGSAPGTSESATPESGPGSPAGSPTSTEEGSPAGSPT |
| | STEEGSPAGSPTSTEEGTSESATPESGPGTSTEPSEGSAPGATRRYYLGAVELSWDYMQSDLGE |
| | LPVDARFPPRVPKSFPFNTSVVYKKTLFVEFTDHLFNIAKPRPPWMGLLGPTIQAEVYDTVVITL |
| | KNMASHPVSLHAVGVSYWKASEGAEYDDQTSQREKEDDKVFPGGSHTYVWQVLKENGPMA |
| | SDPLCLTYSYLSHVDLVKDLNSGLIGALLVCREGSLAKEKTQTLHKFILLFAVFDEGKSWHSET |
| | KNSLMQDRDAASARAWPKMHTVNGYVNRSLPGLIGCHRKSVYWHVIGMGTTPEVHSIFLEG |
| | HTFLVRNHRQASLEISPITFLTAQTLLMDLGQFLLFCHISSHQHDGMEAYVKVDSCPEEPQLRM |
| | KNNEEAEDYDDDLTDSEMDVVRFDDDNSPSFIQIRSVAKKHPKTWVHYIAAEEEDWDYAPLV |
| | LAPDDRSYKSQYLNNGPQRIGRKYKKVRFMAYTDETFKTREAIQHESGILGPLLYGEVGDTLLI |
| | IFKNQASRPYNIYPHGITDVRPLYSRRLPKGVKHLKDFPILPGEIFKYKWTVTVEDGPTKSDPRC |
| | LTRYYSSFVNMERDLASGLIGPLLICYKESVDQRGNQIMSDKRNVILFSVFDENRSWYLTENIQ |
| | RFLPNPAGVQLEDPEFQASNIMHSINGYVFDSLQLSVCLHEVAYWYILSIGAQTDFLSVFFSGY |
| | TFKHKMVYEDTLTLFPFSGETVFMSMENPGLWILGCHNSDFRNRGMTALLKVSSCDKNTGDY |
| | YEDSYEDISAYLLSKNNAIEPRSFSQNSRHPSTRQKQFNATTIPENDIEKTDPWFAHRTPMPKIQ |
| | NVSSSDLLMLLRQSPTPHGLSLSDLQEAKYETFSDDPSPGAIDSNNSLSEMTHFRPQLHHSGDM |
| | VFTPESGLQLRLNEKLGTTAATELKKLDFKVSSTSNNLISTIPSDNLAAGTDNTSSLGPPSMPVH |
| | YDSQLDTTLFGKKSSPLTESGGPLSLSEENNDSKLLESGLMNSQESSWGKNVSSTESGRLFKGK |
| | RAHGPALLTKDNALFKVSISLLKTNKTSNNSATNRKTHIDGPSLLIENSPSVWQNILESDTEFKK |
| | VTPLIHDRMLMDKNATALRLNHMSNKTTSSKNMEMVQQKKEGPIPPDAQNPDMSFFKMLFLP |
| | ESARWIQRTHGKNSLNSGQGPSPKQLVSLGPEKSVEGQNFLSEKNKVVVGKGEFTKDVGLKE |
| | MVFPSSRNLFLTNLDNLHENNTHNQEKKIQEEIEKKETLIQENVVLPQIHTVTGTKNFMKNLFL |
| | LSTRQNVEGSYDGAYAPVLQDFRSLNDSTNRTKKHTAHFSKKGEEENLEGLGNQTKQIVEKY |
| | ACTTRISPNTSQQNFVTQRSKRALKQFRLPLEETELEKRIIVDDTSTQWSKNMKHLTPSTLTQID |
| | YNEKEKGAITQSPLSDCLTRSHSIPQANRSPLPIAKVSSFPSIRPIYLTRVLFQDNSSHLPAASYRK |
| | KDSGVQESSHFLQGAKKNNLSLAILTLEMTGDQREVGSLGTSATNSVTYKKVENTVLPKPDLP |
| | KTSGKVELLPKVHIYQKDLFPTETSNGSPGHLDLVEGSLLQGTEGAIKWNEANRPGKVPFLRV |
| | ATESSAKTPSKLLDPLAWDNHYGTQIPKEEWKSQEKSPEKTAFKKKDTILSLNACESNHAIAAI |
| | NEGQNKPEIEVTWAKQGRTERLCSQNPPVLKRHQREITRTTLQSDQEEIDYDDTISVEMKKEDF |
| | DIYDEDENQSPRSFQKKTRHYFIAAVERLWDYGMSSSPHVLRNRAQSGSVPQFKKVVFQEFTD |
| | GSFTQPLYRGELNEHLGLLGPYIRAEVEDNIMVTFRNQASRPYSFYSSLISYEEDQRQGAEPRK |
| | NFVKPNETKTYFWKVQHHMAPTKDEFDCKAWAYFSDVDLEKDVHSGLIGPLLVCHTNTLNP |
| | AHGRQVTVQEFALFFTIFDETKSWYFTENMERNCRAPCNIQMEDPTFKENYRFHAINGYIMDT |
| | LPGLVMAQDQRIRWYLLSMGSNENIHSIHFSGHVFTVRKKEEYKMALYNLYPGVFETVEMLPS |
| | KAGIWRVECLIGEHLHAGMSTLFLVYSNKCQTPLGMASGHIRDFQITASGQYGQWAPKLARL |
| | HYSGSINAWSTKEPFSWIKVDLLAPMIIHGIKTQGARQKFSSLYISQFIIMYSLDGKKWQTYRGN |
| | STGTLMVFFGNVDSSGIKHNIFNPPIIARYIRLHPTHYSIRSTLRMELMGCDLNSCSMPLGMESK |
| | AISDAQITASSYFTNMFATWSPSKARLHLQGRSNAWRPQVNNPKEWLQVDFQKTMKVTGVTT |
| | QGVKSLLTSMYVKEFLISSSQDGHQWTLFFQNGKVKVFQGNQDSFTPVVNSLDPPLLTRYLRI |
| | HPQSWVHQIALRMEVLGCEAQDLY |
| AF576- | GSTSSTAESPGPGSTSSTAESPGPGSTSESPSGTAPGSTSSTAESPGPGSTSSTAESPGPGTSTPESG |
| FVIII | SASPGSTSESPSGTAPGTSPSGESSTAPGSTSESPSGTAPGSTSESPSGTAPGTSPSGESSTAPGSTS |
| | ESPSGTAPGSTSESPSGTAPGTSPSGESSTAPGSTSESPSGTAPGSTSESPSGTAPGSTSESPSGTAP |
| | GTSTPESGSASPGSTSESPSGTAPGTSTPESGSASPGSTSSTAESPGPGSTSSTAESPGPGTSTPESG |
| | SASPGTSTPESGSASPGSTSESPSGTAPGTSTPESGSASPGTSTPESGSASPGSTSESPSGTAPGSTS |
| | ESPSGTAPGSTSESPSGTAPGSTSSTAESPGPGTSTPESGSASPGTSTPESGSASPGSTSESPSGTAP |
| | GSTSESPSGTAPGTSTPESGSASPGSTSESPSGTAPGSTSESPSGTAPGTSTPESGSASPGTSPSGES |
| | STAPGSTSSTAESPGPGTSPSGESSTAPGSTSSTAESPGPGTSTPESGSASPGSTSESPSGTAPGSTS |
| | STAESPGPGTSTPESGSASPGTSTPESGSASPGATRRYYLGAVELSWDYMQSDLGELPVDARFP |
| | PRVPKSFPFNTSVVYKKTLFVEFTDHLFNIAKPRPPWMGLLGPTIQAEVYDTVVITLKNMASHP |
| | VSLHAVGVSYWKASEGAEYDDQTSQREKEDDKVFPGGSHTYVWQVLKENGPMASDPLCLTY |
| | SYLSHVDLVKDLNSGLIGALLVCREGSLAKEKTQTLHKFILLFAVFDEGKSWHSETKNSLMQD |
| | RDAASARAWPKMHTVNGYVNRSLPGLIGCHRKSVYWHVIGMGTTPEVHSIFLEGHTFLVRNH |
| | RQASLEISPITFLTAQTLLMDLGQFLLFCHISSHQHDGMEAYVKVDSCPEEPQLRMKNNEEAED |
| | YDDDLTDSEMDVVRFDDDNSPSFIQIRSVAKKHPKTWVHYIAAEEEDWDYAPLVLAPDDRSY |
| | KSQYLNNGPQRIGRKYKKVRFMAYTDETFKTREAIQHESGILGPLLYGEVGDTLLIIFKNQASR |
| | PYNIYPHGITDVRPLYSRRLPKGVKHLKDFPILPGEIFKYKWTVTVEDGPTKSDPRCLTRYYSSF |
| | VNMERDLASGLIGPLLICYKESVDQRGNQIMSDKRNVILFSVFDENRSWYLTENIQRFLPNPAG |
| | VQLEDPEFQASNIMHSINGYVFDSLQLSVCLHEVAYWYILSIGAQTDFLSVFFSGYTFKHKMVY |
| | EDTLTLFPFSGETVFMSMENPGLWILGCHNSDFRNRGMTALLKVSSCDKNTGDYYEDSYEDIS |
| | AYLLSKNNAIEPRSFSQNSRHPSTRQKQFNATTIPENDIEKTDPWFAHRTPMPKIQNVSSSDLLM |
| | LLRQSPTPHGLSLSDLQEAKYETFSDDPSPGAIDSNNSLSEMTHFRPQLHHSGDMVFTPESGLQ |
| | LRLNEKLGTTAATELKKLDFKVSSTSNNLISTIPSDNLAAGTDNTSSLGPPSMPVHYDSQLDTTL |
| | FGKKSSPLTESGGPLSLSEENNDSKLLESGLMNSQESSWGKNVSSTESGRLFKGKRAHGPALLT |
| | KDNALFKVSISLLKTNKTSNNSATNRKTHIDGPSLLIENSPSVWQNILESDTEFKKVTPLIHDRM |
| | LMDKNATALRLNHMSNKTTSSKNMEMVQQKKEGPIPPDAQNPDMSFFKMLFLPESARWIQRT |
| | HGKNSLNSGQGPSPKQLVSLGPEKSVEGQNFLSEKNKVVVGKGEFTKDVGLKEMVFPSSRNLF |
| | LTNLDNLHENNTHNQEKKIQEEIEKKETLIQENVVLPQIHTVTGTKNFMKNLFLLSTRQNVEGS |
| | YDGAYAPVLQDFRSLNDSTNRTKKHTAHFSKKGEEENLEGLGNQTKQIVEKYACTTRISPNTS |
| | QQNFVTQRSKRALKQFRLPLEETELEKRIIVDDTSTQWSKNMKHLTPSTLTQIDYNEKEKGAIT |
| | QSPLSDCLTRSHSIPQANRSPLPIAKVSSFPSIRPIYLTRVLFQDNSSHLPAASYRKKDSGVQESS |
| | HFLQGAKKNNLSLAILTLEMTGDQREVGSLGTSATNSVTYKKVENTVLPKPDLPKTSGKVELL |
| | PKVHIYQKDLFPTETSNGSPGHLDLVEGSLLQGTEGAIKWNEANRPGKVPFLRVATESSAKTPS |
| | KLLDPLAWDNHYGTQIPKEEWKSQEKSPEKTAFKKKDTILSLNACESNHAIAAINEGQNKPEIE |
| | VTWAKQGRTERLCSQNPPVLKRHQREITRTTLQSDQEEIDYDDTISVEMKKEDFDIYDEDENQS |
| | PRSFQKKTRHYFIAAVERLWDYGMSSSPHVLRNRAQSGSVPQFKKVVFQEFTDGSFTQPLYRG |
| | ELNEHLGLLGPYIRAEVEDNIMVTFRNQASRPYSFYSSLISYEEDQRQGAEPRKNFVKPNETKT |
| | YFWKVQHHMAPTKDEFDCKAWAYFSDVDLEKDVHSGLIGPLLVCHTNTLNPAHGRQVTVQE |
| | FALFFTIFDETKSWYFTENMERNCRAPCNIQMEDPTFKENYRFHAINGYIMDTLPGLVMAQDQ |
| | RIRWYLLSMGSNENIHSIHFSGHVFTVRKKEEYKMALYNLYPGVFETVEMLPSKAGIWRVECL |
| | IGEHLHAGMSTLFLVYSNKCQTPLGMASGHIRDFQITASGQYGQWAPKLARLHYSGSINAWST |
| | KEPFSWIKVDLLAPMIIHGIKTQGARQKFSSLYISQFIIMYSLDGKKWQTYRGNSTGTLMVFFG |
| | NVDSSGIKHNIFNPPIIARYIRLHPTHYSIRSTLRMELMGCDLNSCSMPLGMESKAISDAQITASS |
| | YFTNMFATWSPSKARLHLQGRSNAWRPQVNNPKEWLQVDFQKTMKVTGVTTQGVKSLLTS |
| | MYVKEFLISSSQDGHQWTLFFQNGKVKVFQGNQDSFTPVVNSLDPPLLTRYLRIHPQSWVHQI |
| | ALRMEVLGCEAQDLY |
| AE864- | GSPAGSPTSTEEGTSESATPESGPGTSTEPSEGSAPGSPAGSPTSTEEGTSTEPSEGSAPGTSTEPS |
| FVIII | EGSAPGTSESATPESGPGSEPATSGSETPGSEPATSGSETPGSPAGSPTSTEEGTSESATPESGPGT |
| | STEPSEGSAPGTSTEPSEGSAPGSPAGSPTSTEEGTSTEPSEGSAPGTSTEPSEGSAPGTSESATPE |
| | SGPGTSTEPSEGSAPGTSESATPESGPGSEPATSGSETPGTSTEPSEGSAPGTSTEPSEGSAPGTSE |
| | SATPESGPGTSESATPESGPGSPAGSPTSTEEGTSESATPESGPGSEPATSGSETPGTSESATPESG |
| | PGTSTEPSEGSAPGTSTEPSEGSAPGTSTEPSEGSAPGTSTEPSEGSAPGTSTEPSEGSAPGTSTEP |
| | SEGSAPGSPAGSPTSTEEGTSTEPSEGSAPGTSESATPESGPGSEPATSGSETPGTSESATPESGPG |
| | SEPATSGSETPGTSESATPESGPGTSTEPSEGSAPGTSESATPESGPGSPAGSPTSTEEGSPAGSPT |
| | STEEGSPAGSPTSTEEGTSESATPESGPGTSTEPSEGSAPGTSESATPESGPGSEPATSGSETPGTS |
| | ESATPESGPGSEPATSGSETPGTSESATPESGPGTSTEPSEGSAPGSPAGSPTSTEEGTSESATPES |
| | GPGSEPATSGSETPGTSESATPESGPGSPAGSPTSTEEGSPAGSPTSTEEGTSTEPSEGSAPGTSES |
| | ATPESGPGTSESATPESGPGTSESATPESGPGSEPATSGSETPGSEPATSGSETPGSPAGSPTSTEE |
| | GTSTEPSEGSAPGTSTEPSEGSAPGSEPATSGSETPGTSESATPESGPGTSTEPSEGSAPGATRRY |
| | YLGAVELSWDYMQSDLGELPVDARFPPRVPKSFPFNTSVVYKKTLFVEFTDHLFNIAKPRPPW |
| | MGLLGPTIQAEVYDTVVITLKNMASHPVSLHAVGVSYWKASEGAEYDDQTSQREKEDDKVFP |
| | GGSHTYVWQVLKENGPMASDPLCLTYSYLSHVDLVKDLNSGLIGALLVCREGSLAKEKTQTL |
| | HKFILLFAVFDEGKSWHSETKNSLMQDRDAASARAWPKMHTVNGYVNRSLPGLIGCHRKSVY |
| | WHVIGMGTTPEVHSIFLEGHTFLVRNHRQASLEISPITFLTAQTLLMDLGQFLLFCHISSHQHDG |
| | MEAYVKVDSCPEEPQLRMKNNEEAEDYDDDLTDSEMDVVRFDDDNSPSFIQIRSVAKKHPKT |
| | WVHYIAAEEEDWDYAPLVLAPDDRSYKSQYLNNGPQRIGRKYKKVRFMAYTDETFKTREAIQ |
| | HESGILGPLLYGEVGDTLLIIFKNQASRPYNIYPHGITDVRPLYSRRLPKGVKHLKDFPILPGEIF |
| | KYKWTVTVEDGPTKSDPRCLTRYYSSFVNMERDLASGLIGPLLICYKESVDQRGNQIMSDKRN |
| | VILFSVFDENRSWYLTENIQRFLPNPAGVQLEDPEFQASNIMHSINGYVFDSLQLSVCLHEVAY |
| | WYILSIGAQTDFLSVFFSGYTFKHKMVYEDTLTLFPFSGETVFMSMENPGLWILGCHNSDFRNR |
| | GMTALLKVSSCDKNTGDYYEDSYEDISAYLLSKNNAIEPRSFSQNSRHPSTRQKQFNATTIPEN |
| | DIEKTDPWFAHRTPMPKIQNVSSSDLLMLLRQSPTPHGLSLSDLQEAKYETFSDDPSPGAIDSN |
| | NSLSEMTHFRPQLHHSGDMVFTPESGLQLRLNEKLGTTAATELKKLDFKVSSTSNNLISTIPSDN |
| | LAAGTDNTSSLGPPSMPVHYDSQLDTTLFGKKSSPLTESGGPLSLSEENNDSKLLESGLMNSQE |
| | SSWGKNVSSTESGRLFKGKRAHGPALLTKDNALFKVSISLLKTNKTSNNSATNRKTHIDGPSLL |
| | IENSPSVWQNILESDTEFKKVTPLIHDRMLMDKNATALRLNHMSNKTTSSKNMEMVQQKKEG |
| | PIPPDAQNPDMSFFKMLFLPESARWIQRTHGKNSLNSGQGPSPKQLVSLGPEKSVEGQNFLSEK |
| | NKVVVGKGEFTKDVGLKEMVFPSSRNLFLTNLDNLHENNTHNQEKKIQEEIEKKETLIQENVV |
| | LPQIHTVTGTKNFMKNLFLLSTRQNVEGSYDGAYAPVLQDFRSLNDSTNRTKKHTAHFSKKGE |
| | EENLEGLGNQTKQIVEKYACTTRISPNTSQQNFVTQRSKRALKQFRLPLEETELEKRIIVDDTST |
| | QWSKNMKHLTPSTLTQIDYNEKEKGAITQSPLSDCLTRSHSIPQANRSPLPIAKVSSFPSIRPIYL |
| | TRVLFQDNSSHLPAASYRKKDSGVQESSHFLQGAKKNNLSLAILTLEMTGDQREVGSLGTSAT |
| | NSVTYKKVENTVLPKPDLPKTSGKVELLPKVHIYQKDLFPTETSNGSPGHLDLVEGSLLQGTEG |
| | AIKWNEANRPGKVPFLRVATESSAKTPSKLLDPLAWDNHYGTQIPKEEWKSQEKSPEKTAFKK |
| | KDTILSLNACESNHAIAAINEGQNKPEIEVTWAKQGRTERLCSQNPPVLKRHQREITRTTLQSD |
| | QEEIDYDDTISVEMKKEDFDIYDEDENQSPRSFQKKTRHYFIAAVERLWDYGMSSSPHVLRNR |
| | AQSGSVPQFKKVVFQEFTDGSFTQPLYRGELNEHLGLLGPYIRAEVEDNIMVTFRNQASRPYSF |
| | YSSLISYEEDQRQGAEPRKNFVKPNETKTYFWKVQHHMAPTKDEFDCKAWAYFSDVDLEKD |
| | VHSGLIGPLLVCHTNTLNPAHGRQVTVQEFALFFTIFDETKSWYFTENMERNCRAPCNIQMEDP |
| | TFKENYRFHAINGYIMDTLPGLVMAQDQRIRWYLLSMGSNENIHSIHFSGHVFTVRKKEEYKM |
| | ALYNLYPGVFETVEMLPSKAGIWRVECLIGEHLHAGMSTLFLVYSNKCQTPLGMASGHIRDFQ |
| | ITASGQYGQWAPKLARLHYSGSINAWSTKEPFSWIKVDLLAPMIIHGIKTQGARQKFSSLYISQF |
| | IIMYSLDGKKWQTYRGNSTGTLMVFFGNVDSSGIKHNIFNPPIIARYIRLHPTHYSIRSTLRMEL |
| | MGCDLNSCSMPLGMESKAISDAQITASSYFTNMFATWSPSKARLHLQGRSNAWRPQVNNPKE |
| | WLQVDFQKTMKVTGVTTQGVKSLLTSMYVKEFLISSSQDGHQWTLFFQNGKVKVFQGNQDS |
| | FTPVVNSLDPPLLTRYLRIHPQSWVHQIALRMEVLGCEAQDLY |
| AF864- | GSTSESPSGTAPGTSPSGESSTAPGSTSESPSGTAPGSTSESPSGTAPGTSTPESGSASPGTSTPESG |
| FVIII | SASPGSTSESPSGTAPGSTSESPSGTAPGTSPSGESSTAPGSTSESPSGTAPGTSPSGESSTAPGTSP |
| | SGESSTAPGSTSSTAESPGPGTSPSGESSTAPGTSPSGESSTAPGSTSSTAESPGPGTSTPESGSASP |
| | GTSTPESGSASPGSTSESPSGTAPGSTSESPSGTAPGTSTPESGSASPGSTSSTAESPGPGTSTPESG |
| | SASPGSTSESPSGTAPGTSPSGESSTAPGSTSSTAESPGPGTSPSGESSTAPGTSTPESGSASPGSTS |
| | STAESPGPGSTSSTAESPGPGSTSSTAESPGPGSTSSTAESPGPGTSPSGESSTAPGSTSESPSGTAP |
| | GSTSESPSGTAPGTSTPESGPXXXGASASGAPSTXXXXSESPSGTAPGSTSESPSGTAPGSTSESP |
| | SGTAPGSTSESPSGTAPGSTSESPSGTAPGSTSESPSGTAPGTSTPESGSASPGTSPSGESSTAPGT |
| | SPSGESSTAPGSTSSTAESPGPGTSPSGESSTAPGTSTPESGSASPGSTSESPSGTAPGSTSESPSGT |
| | APGTSPSGESSTAPGSTSESPSGTAPGTSTPESGSASPGTSTPESGSASPGSTSESPSGTAPGTSTPE |
| | SGSASPGSTSSTAESPGPGSTSESPSGTAPGSTSESPSGTAPGTSPSGESSTAPGSTSSTAESPGPG |
| | TSPSGESSTAPGTSTPESGSASPGTSPSGESSTAPGTSPSGESSTAPGTSPSGESSTAPGSTSSTAES |
| | PGPGSTSSTAESPGPGTSPSGESSTAPGSSPSASTGTGPGSSTPSGATGSPGSSTPSGATGSPGATR |
| | RYYLGAVELSWDYMQSDLGELPVDARFPPRVPKSFPFNTSVVYKKTLFVEFTDHLFNIAKPRP |
| | PWMGLLGPTIQAEVYDTVVITLKNMASHPVSLHAVGVSYWKASEGAEYDDQTSQREKEDDK |
| | VFPGGSHTYVWQVLKENGPMASDPLCLTYSYLSHVDLVKDLNSGLIGALLVCREGSLAKEKT |
| | QTLHKFILLFAVFDEGKSWHSETKNSLMQDRDAASARAWPKMHTVNGYVNRSLPGLIGCHRK |
| | SVYWHVIGMGTTPEVHSIFLEGHTFLVRNHRQASLEISPITFLTAQTLLMDLGQFLLFCHISSHQ |
| | HDGMEAYVKVDSCPEEPQLRMKNNEEAEDYDDDLTDSEMDVVRFDDDNSPSFIQIRSVAKKH |
| | PKTWVHYIAAEEEDWDYAPLVLAPDDRSYKSQYLNNGPQRIGRKYKKVRFMAYTDETFKTRE |
| | AIQHESGILGPLLYGEVGDTLLIIFKNQASRPYNIYPHGITDVRPLYSRRLPKGVKHLKDFPILPG |
| | EIFKYKWTVTVEDGPTKSDPRCLTRYYSSFVNMERDLASGLIGPLLICYKESVDQRGNQIMSDK |
| | RNVILFSVFDENRSWYLTENIQRFLPNPAGVQLEDPEFQASNIMHSINGYVFDSLQLSVCLHEV |
| | AYWYILSIGAQTDFLSVFFSGYTFKHKMVYEDTLTLFPFSGETVFMSMENPGLWILGCHNSDF |
| | RNRGMTALLKVSSCDKNTGDYYEDSYEDISAYLLSKNNAIEPRSFSQNSRHPSTRQKQFNATTI |
| | PENDIEKTDPWFAHRTPMPKIQNVSSSDLLMLLRQSPTPHGLSLSDLQEAKYETFSDDPSPGAID |
| | SNNSLSEMTHFRPQLHHSGDMVFTPESGLQLRLNEKLGTTAATELKKLDFKVSSTSNNLISTIPS |
| | DNLAAGTDNTSSLGPPSMPVHYDSQLDTTLFGKKSSPLTESGGPLSLSEENNDSKLLESGLMNS |
| | QESSWGKNVSSTESGRLFKGKRAHGPALLTKDNALFKVSISLLKTNKTSNNSATNRKTHIDGPS |
| | LLIENSPSVWQNILESDTEFKKVTPLIHDRMLMDKNATALRLNHMSNKTTSSKNMEMVQQKK |
| | EGPIPPDAQNPDMSFFKMLFLPESARWIQRTHGKNSLNSGQGPSPKQLVSLGPEKSVEGQNFLS |
| | EKNKVVVGKGEFTKDVGLKEMVFPSSRNLFLTNLDNLHENNTHNQEKKIQEEIEKKETLIQEN |
| | VVLPQIHTVTGTKNFMKNLFLLSTRQNVEGSYDGAYAPVLQDFRSLNDSTNRTKKHTAHFSK |
| | KGEEENLEGLGNQTKQIVEKYACTTRISPNTSQQNFVTQRSKRALKQFRLPLEETELEKRIIVDD |
| | TSTQWSKNMKHLTPSTLTQIDYNEKEKGAITQSPLSDCLTRSHSIPQANRSPLPIAKVSSFPSIRPI |
| | YLTRVLFQDNSSHLPAASYRKKDSGVQESSHFLQGAKKNNLSLAILTLEMTGDQREVGSLGTS |
| | ATNSVTYKKVENTVLPKPDLPKTSGKVELLPKVHIYQKDLFPTETSNGSPGHLDLVEGSLLQGT |
| | EGAIKWNEANRPGKVPFLRVATESSAKTPSKLLDPLAWDNHYGTQIPKEEWKSQEKSPEKTAF |
| | KKKDTILSLNACESNHAIAAINEGQNKPEIEVTWAKQGRTERLCSQNPPVLKRHQREITRTTLQ |
| | SDQEEIDYDDTISVEMKKEDFDIYDEDENQSPRSFQKKTRHYFIAAVERLWDYGMSSSPHVLR |
| | NRAQSGSVPQFKKVVFQEFTDGSFTQPLYRGELNEHLGLLGPYIRAEVEDNIMVTFRNQASRP |
| | YSFYSSLISYEEDQRQGAEPRKNFVKPNETKTYFWKVQHHMAPTKDEFDCKAWAYFSDVDLE |
| | KDVHSGLIGPLLVCHTNTLNPAHGRQVTVQEFALFFTIFDETKSWYFTENMERNCRAPCNIQM |
| | EDPTFKENYRFHAINGYIMDTLPGLVMAQDQRIRWYLLSMGSNENIHSIHFSGHVFTVRKKEE |
| | YKMALYNLYPGVFETVEMLPSKAGIWRVECLIGEHLHAGMSTLFLVYSNKCQTPLGMASGHI |
| | RDFQITASGQYGQWAPKLARLHYSGSINAWSTKEPFSWIKVDLLAPMIIHGIKTQGARQKFSSL |
| | YISQFIIMYSLDGKKWQTYRGNSTGTLMVFFGNVDSSGIKHNIFNPPIIARYIRLHPTHYSIRSTL |
| | RMELMGCDLNSCSMPLGMESKAISDAQITASSYFTNMFATWSPSKARLHLQGRSNAWRPQVN |
| | NPKEWLQVDFQKTMKVTGVTTQGVKSLLTSMYVKEFLISSSQDGHQWTLFFQNGKVKVFQG |
| | NQDSFTPVVNSLDPPLLTRYLRIHPQSWVHQIALRMEVLGCEAQDLY |
| AG864- | GASPGTSSTGSPGSSPSASTGTGPGSSPSASTGTGPGTPGSGTASSSPGSSTPSGATGSPGSNPSAS |
| FVIII | TGTGPGASPGTSSTGSPGTPGSGTASSSPGSSTPSGATGSPGTPGSGTASSSPGASPGTSSTGSPG |
| | ASPGTSSTGSPGTPGSGTASSSPGSSTPSGATGSPGASPGTSSTGSPGTPGSGTASSSPGSSTPSGA |
| | TGSPGSNPSASTGTGPGSSPSASTGTGPGSSTPSGATGSPGSSTPSGATGSPGASPGTSSTGSPGA |
| | SPGTSSTGSPGASPGTSSTGSPGTPGSGTASSSPGASPGTSSTGSPGASPGTSSTGSPGASPGTSST |
| | GSPGSSPSASTGTGPGTPGSGTASSSPGASPGTSSTGSPGASPGTSSTGSPGASPGTSSTGSPGSST |
| | PSGATGSPGSSTPSGATGSPGASPGTSSTGSPGTPGSGTASSSPGSSTPSGATGSPGSSTPSGATG |
| | SPGSSTPSGATGSPGSSPSASTGTGPGASPGTSSTGSPGASPGTSSTGSPGTPGSGTASSSPGASPG |
| | TSSTGSPGASPGTSSTGSPGASPGTSSTGSPGASPGTSSTGSPGTPGSGTASSSPGSSTPSGATGSP |
| | GTPGSGTASSSPGSSTPSGATGSPGTPGSGTASSSPGSSTPSGATGSPGSSTPSGATGSPGSSPSAS |
| | TGTGPGSSPSASTGTGPGASPGTSSTGSPGTPGSGTASSSPGSSTPSGATGSPGSSPSASTGTGPG |
| | SSPSASTGTGPGASPGTSSTGSPGASPGTSSTGSPGSSTPSGATGSPGSSPSASTGTGPGASPGTSS |
| | TGSPGSSPSASTGTGPGTPGSGTASSSPGSSTPSGATGSPGSSTPSGATGSPGASPGTSSTGSPGA |
| | TRRYYLGAVELSWDYMQSDLGELPVDARFPPRVPKSFPFNTSVVYKKTLFVEFTDHLFNIAKP |
| | RPPWMGLLGPTIQAEVYDTVVITLKNMASHPVSLHAVGVSYWKASEGAEYDDQTSQREKED |
| | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNSRHPSTRQKQFN |
| | ATTIPENDIEKTDPWFAHRTPMPKIQNVSSSDLLMLLRQSPTPHGLSLSDLQEAKYETFSDDPSP |
| | GAIDSNNSLSEMTHFRPQLHHSGDMVFTPESGLQLRLNEKLGTTAATELKKLDFKVSSTSNNLI |
| | STIPSDNLAAGTDNTSSLGPPSMPVHYDSQLDTTLFGKKSSPLTESGGPLSLSEENNDSKLLESG |
| | LMNSQESSWGKNVSSTESGRLFKGKRAHGPALLTKDNALFKVSISLLKTNKTSNNSATNRKTH |
| | IDGPSLLIENSPSVWQNILESDTEFKKVTPLIHDRMLMDKNATALRLNHMSNKTTSSKNMEMV |
| | QQKKEGPIPPDAQNPDMSFFKMLFLPESARWIQRTHGKNSLNSGQGPSPKQLVSLGPEKSVEG |
| | QNFLSEKNKVVVGKGEFTKDVGLKEMVFPSSRNLFLTNLDNLHENNTHNQEKKIQEEIEKKET |
| | LIQENVVLPQIHTVTGTKNFMKNLFLLSTRQNVEGSYDGAYAPVLQDFRSLNDSTNRTKKHTA |
| | HFSKKGEEENLEGLGNQTKQIVEKYACTTRISPNTSQQNFVTQRSKRALKQFRLPLEETELEKRI |
| | IVDDTSTQWSKNMKHLTPSTLTQIDYNEKEKGAITQSPLSDCLTRSHSIPQANRSPLPIAKVSSFP |
| | SIRPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEGSL |
| | LQGTEGAIKWNEANRPGKVPFLRVATESSAKTPSKLLDPLAWDNHYGTQIPKEEWKSQEKSPE |
| | KTAFKKKDTILSLNACESNHAIAAINEGQNKPEIEVTWAKQGRTERLCSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLY |
| AM875- | GTSTEPSEGSAPGSEPATSGSETPGSPAGSPTSTEEGSTSSTAESPGPGTSTPESGSASPGSTSESPS |
| FVIII | GTAPGSTSESPSGTAPGTSTPESGSASPGTSTPESGSASPGSEPATSGSETPGTSESATPESGPGSP |
| | AGSPTSTEEGTSTEPSEGSAPGTSESATPESGPGTSTEPSEGSAPGTSTEPSEGSAPGSPAGSPTST |
| | EEGTSTEPSEGSAPGTSTEPSEGSAPGTSESATPESGPGTSESATPESGPGTSTEPSEGSAPGTSTE |
| | PSEGSAPGTSESATPESGPGTSTEPSEGSAPGSEPATSGSETPGSPAGSPTSTEEGSSTPSGATGSP |
| | GTPGSGTASSSPGSSTPSGATGSPGTSTEPSEGSAPGTSTEPSEGSAPGSEPATSGSETPGSPAGSP |
| | TSTEEGSPAGSPTSTEEGTSTEPSEGSAPGASASGAPSTGGTSESATPESGPGSPAGSPTSTEEGSP |
| | AGSPTSTEEGSTSSTAESPGPGSTSESPSGTAPGTSPSGESSTAPGTPGSGTASSSPGSSTPSGATG |
| | SPGSSPSASTGTGPGSEPATSGSETPGTSESATPESGPGSEPATSGSETPGSTSSTAESPGPGSTSS |
| | TAESPGPGTSPSGESSTAPGSEPATSGSETPGSEPATSGSETPGTSTEPSEGSAPGSTSSTAESPGP |
| | GTSTPESGSASPGSTSESPSGTAPGTSTEPSEGSAPGTSTEPSEGSAPGTSTEPSEGSAPGSSTPSG |
| | ATGSPGSSPSASTGTGPGASPGTSSTGSPGSEPATSGSETPGTSESATPESGPGSPAGSPTSTEEGS |
| | STPSGATGSPGSSPSASTGTGPGASPGTSSTGSPGTSESATPESGPGTSTEPSEGSAPGTSTEPSEG |
| | SAPGATRRYYLGAVELSWDYMQSDLGELPVDARFPPRVPKSFPFNTSVVYKKTLFVEFTDHLF |
| | NIAKPRPPWMGLLGPTIQAEVYDTVVITLKNMASHPVSLHAVGVSYWKASEGAEYDDQTSQR |
| | EKEDDKVFPGGSHTYVWQVLKENGPMASDPLCLTYSYLSHVDLVKDLNSGLIGALLVCREGS |
| | LAKEKTQTLHKFILLFAVFDEGKSWHSETKNSLMQDRDAASARAWPKMHTVNGYVNRSLPG |
| | LIGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQIR |
| | SVAKKHPKTWVHYIAAEEEDWDYAPLVLAPDDRSYKSQYLNNGPQRIGRKYKKVRFMAYTD |
| | ETFKTREAIQHESGILGPLLYGEVGDTLLIIFKNQASRPYNIYPHGITDVRPLYSRRLPKGVKHLK |
| | DFPILPGEIFKYKWTVTVEDGPTKSDPRCLTRYYSSFVNMERDLASGLIGPLLICYKESVDQRG |
| | NQIMSDKRNVILFSVFDENRSWYLTENIQRFLPNPAGVQLEDPEFQASNIMHSINGYVFDSLQL |
| | SVCLHEVAYWYILSIGAQTDFLSVFFSGYTFKHKMVYEDTLTLFPFSGETVFMSMENPGLWILG |
| | CHNSDFRNRGMTALLKVSSCDKNTGDYYEDSYEDISAYLLSKNNAIEPRSFSQNSRHPSTRQK |
| | QFNATTIPENDIEKTDPWFAHRTPMPKIQNVSSSDLLMLLRQSPTPHGLSLSDLQEAKYETFSDD |
| | PSPGAIDSNNSLSEMTHFRPQLHHSGDMVFTPESGLQLRLNEKLGTTAATELKKLDFKVSSTSN |
| | NLISTIPSDNLAAGTDNTSSLGPPSMPVHYDSQLDTTLFGKKSSPLTESGGPLSLSEENNDSKLL |
| | ESGLMNSQESSWGKNVSSTESGRLFKGKRAHGPALLTKDNALFKVSISLLKTNKTSNNSATNR |
| | KTHIDGPSLLIENSPSVWQNILESDTEFKKVTPLIHDRMLMDKNATALRLNHMSNKTTSSKNM |
| | EMVQQKKEGPIPPDAQNPDMSFFKMLFLPESARWIQRTHGKNSLNSGQGPSPKQLVSLGPEKS |
| | VEGQNFLSEKNKVVVGKGEFTKDVGLKEMVFPSSRNLFLTNLDNLHENNTHNQEKKIQEEIEK |
| | KETLIQENVVLPQIHTVTGTKNFMKNLFLLSTRQNVEGSYDGAYAPVLQDFRSLNDSTNRTKK |
| | HTAHFSKKGEEENLEGLGNQTKQIVEKYACTTRISPNTSQQNFVTQRSKRALKQFRLPLEETEL |
| | EKRIIVDDTSTQWSKNMKHLTPSTLTQIDYNEKEKGAITQSPLSDCLTRSHSIPQANRSPLPIAKV |
| | SSFPSIRPIYLTRVLFQDNSSHLPAASYRKKDSGVQESSHFLQGAKKNNLSLAILTLEMTGDQRE |
| | VGSLGTSATNSVTYKKVENTVLPKPDLPKTSGKVELLPKVHIYQKDLFPTETSNGSPGHLDLVE |
| | GSLLQGTEGAIKWNEANRPGKVPFLRVATESSAKTPSKLLDPLAWDNHYGTQIPKEEWKSQEK |
| | SPEKTAFKKKDTILSLNACESNHAIAAINEGQNKPEIEVTWAKQGRTERLCSQNPPVLKRHQRE |
| | ITRTTLQSDQEEIDYDDTISVEMKKEDFDIYDEDENQSPRSFQKKTRHYFIAAVERLWDYGMSS |
| | SPHVLRNRAQSGSVPQFKKVVFQEFTDGSFTQPLYRGELNEHLGLLGPYIRAEVEDNIMVTFRN |
| | QASRPYSFYSSLISYEEDQRQGAEPRKNFVKPNETKTYFWKVQHHMAPTKDEFDCKAWAYFS |
| | DVDLEKDVHSGLIGPLLVCHTNTLNPAHGRQVTVQEFALFFTIFDETKSWYFTENMERNCRAP |
| | CNIQMEDPTFKENYRFHAINGYIMDTLPGLVMAQDQRIRWYLLSMGSNENIHSIHFSGHVFTV |
| | RKKEEYKMALYNLYPGVFETVEMLPSKAGIWRVECLIGEHLHAGMSTLFLVYSNKCQTPLGM |
| | ASGHIRDFQITASGQYGQWAPKLARLHYSGSINAWSTKEPFSWIKVDLLAPMIIHGIKTQGARQ |
| | KFSSLYISQFIIMYSLDGKKWQTYRGNSTGTLMVFFGNVDSSGIKHNIFNPPIIARYIRLHPTHYS |
| | IRSTLRMELMGCDLNSCSMPLGMESKAISDAQITASSYFTNMFATWSPSKARLHLQGRSNAWR |
| | PQVNNPKEWLQVDFQKTMKVTGVTTQGVKSLLTSMYVKEFLISSSQDGHQWTLFFQNGKVK |
| | VFQGNQDSFTPVVNSLDPPLLTRYLRIHPQSWVHQIALRMEVLGCEAQDLY |
| FVIII | ATRRYYLGAVELSWDYMQSDLGELPVDARFPPRVPKSFPFNTSVVYKKTLFVEFTVHLFNIAK |
| BDD2- | PRPPWMGLLGPTIQAEVYDTVVITLKNMASHPVSLHAVGVSYWKASEGAEYDDQTSQREKED |
| AE864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSPAGSPTSTEEGT |
| | SESATPESGPGTSTEPSEGSAPGSPAGSPTSTEEGTSTEPSEGSAPGTSTEPSEGSAPGTSESATPE |
| | SGPGSEPATSGSETPGSEPATSGSETPGSPAGSPTSTEEGTSESATPESGPGTSTEPSEGSAPGTST |
| | EPSEGSAPGSPAGSPTSTEEGTSTEPSEGSAPGTSTEPSEGSAPGTSESATPESGPGTSTEPSEGSA |
| | PGTSESATPESGPGSEPATSGSETPGTSTEPSEGSAPGTSTEPSEGSAPGTSESATPESGPGTSESA |
| | TPESGPGSPAGSPTSTEEGTSESATPESGPGSEPATSGSETPGTSESATPESGPGTSTEPSEGSAPG |
| | TSTEPSEGSAPGTSTEPSEGSAPGTSTEPSEGSAPGTSTEPSEGSAPGTSTEPSEGSAPGSPAGSPT |
| | STEEGTSTEPSEGSAPGTSESATPESGPGSEPATSGSETPGTSESATPESGPGSEPATSGSETPGTS |
| | ESATPESGPGTSTEPSEGSAPGTSESATPESGPGSPAGSPTSTEEGSPAGSPTSTEEGSPAGSPTST |
| | EEGTSESATPESGPGTSTEPSEGSAPGTSESATPESGPGSEPATSGSETPGTSESATPESGPGSEPA |
| | TSGSETPGTSESATPESGPGTSTEPSEGSAPGSPAGSPTSTEEGTSESATPESGPGSEPATSGSETP |
| | GTSESATPESGPGSPAGSPTSTEEGSPAGSPTSTEEGTSTEPSEGSAPGTSESATPESGPGTSESAT |
| | PESGPGTSESATPESGPGSEPATSGSETPGSEPATSGSETPGSPAGSPTSTEEGTSTEPSEGSAPGT |
| | STEPSEGSAPGSEPATSGSETPGTSESATPESGPGTSTEPSEGSAP |
| FVIII | ATRRYYLGAVELSWDYMQSDLGELPVDARFPPRVPKSFPFNTSVVYKKTLFVEFTVHLFNIAK |
| BDD2- | PRPPWMGLLGPTIQAEVYDTVVITLKNMASHPVSLHAVGVSYWKASEGAEYDDQTSQREKED |
| AG864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGASPGTSSTGSPG |
| | SSPSASTGTGPGSSPSASTGTGPGTPGSGTASSSPGSSTPSGATGSPGSNPSASTGTGPGASPGTS |
| | STGSPGTPGSGTASSSPGSSTPSGATGSPGTPGSGTASSSPGASPGTSSTGSPGASPGTSSTGSPGT |
| | PGSGTASSSPGSSTPSGATGSPGASPGTSSTGSPGTPGSGTASSSPGSSTPSGATGSPGSNPSASTG |
| | TGPGSSPSASTGTGPGSSTPSGATGSPGSSTPSGATGSPGASPGTSSTGSPGASPGTSSTGSPGAS |
| | PGTSSTGSPGTPGSGTASSSPGASPGTSSTGSPGASPGTSSTGSPGASPGTSSTGSPGSSPSASTGT |
| | GPGTPGSGTASSSPGASPGTSSTGSPGASPGTSSTGSPGASPGTSSTGSPGSSTPSGATGSPGSSTP |
| | SGATGSPGASPGTSSTGSPGTPGSGTASSSPGSSTPSGATGSPGSSTPSGATGSPGSSTPSGATGS |
| | PGSSPSASTGTGPGASPGTSSTGSPGASPGTSSTGSPGTPGSGTASSSPGASPGTSSTGSPGASPG |
| | TSSTGSPGASPGTSSTGSPGASPGTSSTGSPGTPGSGTASSSPGSSTPSGATGSPGTPGSGTASSSP |
| | GSSTPSGATGSPGTPGSGTASSSPGSSTPSGATGSPGSSTPSGATGSPGSSPSASTGTGPGSSPSAS |
| | TGTGPGASPGTSSTGSPGTPGSGTASSSPGSSTPSGATGSPGSSPSASTGTGPGSSPSASTGTGPG |
| | ASPGTSSTGSPGASPGTSSTGSPGSSTPSGATGSPGSSPSASTGTGPGASPGTSSTGSPGSSPSAST |
| | GTGPGTPGSGTASSSPGSSTPSGATGSPGSSTPSGATGSPGASPGTSSTGSP |
| FVIII | ATRRYYLGAVELSWDYMQSDLGELPVDARFPPRVPKSFPFNTSVVYKKTLFVEFTVHLFNIAK |
| BDD3- | PRPPWMGLLGPTIQAEVYDTVVITLKNMASHPVSLHAVGVSYWKASEGAEYDDQTSQREKED |
| AE576 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQGEITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSPAGSPTSTEEGT |
| | SESATPESGPGTSTEPSEGSAPGSPAGSPTSTEEGTSTEPSEGSAPGTSTEPSEGSAPGTSESATPE |
| | SGPGSEPATSGSETPGSEPATSGSETPGSPAGSPTSTEEGTSESATPESGPGTSTEPSEGSAPGTST |
| | EPSEGSAPGSPAGSPTSTEEGTSTEPSEGSAPGTSTEPSEGSAPGTSESATPESGPGTSTEPSEGSA |
| | PGTSESATPESGPGSEPATSGSETPGTSTEPSEGSAPGTSTEPSEGSAPGTSESATPESGPGTSESA |
| | TPESGPGSPAGSPTSTEEGTSESATPESGPGSEPATSGSETPGTSESATPESGPGTSTEPSEGSAPG |
| | TSTEPSEGSAPGTSTEPSEGSAPGTSTEPSEGSAPGTSTEPSEGSAPGTSTEPSEGSAPGSPAGSPT |
| | STEEGTSTEPSEGSAPGTSESATPESGPGSEPATSGSETPGTSESATPESGPGSEPATSGSETPGTS |
| | ESATPESGPGTSTEPSEGSAPGTSESATPESGPGSPAGSPTSTEEGSPAGSPTSTEEGSPAGSPTST |
| | EEGTSESATPESGPGTSTEPSEGSAP |
| FVIII | ATRRYYLGAVELSWDYMQSDLGELPVDARFPPRVPKSFPFNTSVVYKKTLFVEFTVHLFNIAK |
| BDD4- | PRPPWMGLLGPTIQAEVYDTVVITLKNMASHPVSLHAVGVSYWKASEGAEYDDQTSQREKED |
| AE864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQQSPRSFQKKTRHYF |
| | IAAVERLWDYGMSSSPHVLRNRAQSGSVPQFKKVVFQEFTDGSFTQPLYRGELNEHLGLLGPY |
| | IRAEVEDNIMVTFRNQASRPYSFYSSLISYEEDQRQGAEPRKNFVKPNETKTYFWKVQHHMAP |
| | TKDEFDCKAWAYFSDVDLEKDVHSGLIGPLLVCHTNTLNPAHGRQVTVQEFALFFTIFDETKS |
| | WYFTENMERNCRAPCNIQMEDPTFKENYRFHAINGYIMDTLPGLVMAQDQRIRWYLLSMGSN |
| | ENIHSIHFSGHVFTVRKKEEYKMALYNLYPGVFETVEMLPSKAGIWRVECLIGEHLHAGMSTL |
| | FLVYSNKCQTPLGMASGHIRDFQITASGQYGQWAPKLARLHYSGSINAWSTKEPFSWIKVDLL |
| | APMIIHGIKTQGARQKFSSLYISQFIIMYSLDGKKWQTYRGNSTGTLMVFFGNVDSSGIKHNIFN |
| | PPIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEAQ |
| | DLYGGSPAGSPTSTEEGTSESATPESGPGTSTEPSEGSAPGSPAGSPTSTEEGTSTEPSEGSAPGTS |
| | TEPSEGSAPGTSESATPESGPGSEPATSGSETPGSEPATSGSETPGSPAGSPTSTEEGTSESATPES |
| | GPGTSTEPSEGSAPGTSTEPSEGSAPGSPAGSPTSTEEGTSTEPSEGSAPGTSTEPSEGSAPGTSES |
| | ATPESGPGTSTEPSEGSAPGTSESATPESGPGSEPATSGSETPGTSTEPSEGSAPGTSTEPSEGSAP |
| | GTSESATPESGPGTSESATPESGPGSPAGSPTSTEEGTSESATPESGPGSEPATSGSETPGTSESAT |
| | PESGPGTSTEPSEGSAPGTSTEPSEGSAPGTSTEPSEGSAPGTSTEPSEGSAPGTSTEPSEGSAPGT |
| | STEPSEGSAPGSPAGSPTSTEEGTSTEPSEGSAPGTSESATPESGPGSEPATSGSETPGTSESATPE |
| | SGPGSEPATSGSETPGTSESATPESGPGTSTEPSEGSAPGTSESATPESGPGSPAGSPTSTEEGSPA |
| | GSPTSTEEGSPAGSPTSTEEGTSESATPESGPGTSTEPSEGSAPGTSESATPESGPGSEPATSGSET |
| | PGTSESATPESGPGSEPATSGSETPGTSESATPESGPGTSTEPSEGSAPGSPAGSPTSTEEGTSESA |
| | TPESGPGSEPATSGSETPGTSESATPESGPGSPAGSPTSTEEGSPAGSPTSTEEGTSTEPSEGSAPG |
| | TSESATPESGPGTSESATPESGPGTSESATPESGPGSEPATSGSETPGSEPATSGSETPGSPAGSPT |
| | STEEGTSTEPSEGSAPGTSTEPSEGSAPGSEPATSGSETPGTSESATPESGPGTSTEPSEGSAP |
| AE912- | MAEPAGSPTSTEEGTPGSGTASSSPGSSTPSGATGSPGASPGTSSTGSPGSPAGSPTSTEEGTSES |
| FVIII | ATPESGPGTSTEPSEGSAPGSPAGSPTSTEEGTSTEPSEGSAPGTSTEPSEGSAPGTSESATPESGP |
| BDD9 | GSEPATSGSETPGSEPATSGSETPGSPAGSPTSTEEGTSESATPESGPGTSTEPSEGSAPGTSTEPS |
| | EGSAPGSPAGSPTSTEEGTSTEPSEGSAPGTSTEPSEGSAPGTSESATPESGPGTSTEPSEGSAPGT |
| | SESATPESGPGSEPATSGSETPGTSTEPSEGSAPGTSTEPSEGSAPGTSESATPESGPGTSESATPE |
| | SGPGSPAGSPTSTEEGTSESATPESGPGSEPATSGSETPGTSESATPESGPGTSTEPSEGSAPGTST |
| | EPSEGSAPGTSTEPSEGSAPGTSTEPSEGSAPGTSTEPSEGSAPGTSTEPSEGSAPGSPAGSPTSTE |
| | EGTSTEPSEGSAPGTSESATPESGPGSEPATSGSETPGTSESATPESGPGSEPATSGSETPGTSESA |
| | TPESGPGTSTEPSEGSAPGTSESATPESGPGSPAGSPTSTEEGSPAGSPTSTEEGSPAGSPTSTEEG |
| | TSESATPESGPGTSTEPSEGSAPGTSESATPESGPGSEPATSGSETPGTSESATPESGPGSEPATSG |
| | SETPGTSESATPESGPGTSTEPSEGSAPGSPAGSPTSTEEGTSESATPESGPGSEPATSGSETPGTS |
| | ESATPESGPGSPAGSPTSTEEGSPAGSPTSTEEGTSTEPSEGSAPGTSESATPESGPGTSESATPES |
| | GPGTSESATPESGPGSEPATSGSETPGSEPATSGSETPGSPAGSPTSTEEGTSTEPSEGSAPGTSTE |
| | PSEGSAPGSEPATSGSETPGTSESATPESGPGTSTEPSEGSAPGATRRYYLGAVELSWDYMQSD |
| | LGELPVDARFPPRVPKSFPFNTSVVYKKTLFVEFTDHLFNIAKPRPPWMGLLGPTIQAEVYDTV |
| | VITLKNMASHPVSLHAVGVSYWKASEGAEYDDQTSQREKEDDKVFPGGSHTYVWQVLKENG |
| | PMASDPLCLTYSYLSHVDLVKDLNSGLIGALLVCREGSLAKEKTQTLHKFILLFAVFDEGKSW |
| | HSETKNSLMQDRDAASARAWPKMHTVNGYVNRSLPGLIGCHRKSVYWHVIGMGTTPEVHSIF |
| | LEGHTFLVRNHRQASLEISPITFLTAQTLLMDLGQFLLFCHISSHQHDGMEAYVKVDSCPEEPQ |
| | LRMKNNEEAEDYDDDLTDSEMDVVRFDDDNSPSFIQIRSVAKKHPKTWVHYIAAEEEDWDYA |
| | PLVLAPDDRSYKSQYLNNGPQRIGRKYKKVRFMAYTDETFKTREAIQHESGILGPLLYGEVGD |
| | TLLIIFKNQASRPYNIYPHGITDVRPLYSRRLPKGVKHLKDFPILPGEIFKYKWTVTVEDGPTKS |
| | DPRCLTRYYSSFVNMERDLASGLIGPLLICYKESVDQRGNQIMSDKRNVILFSVFDENRSWYLT |
| | ENIQRFLPNPAGVQLEDPEFQASNIMHSINGYVFDSLQLSVCLHEVAYWYILSIGAQTDFLSVFF |
| | SGYTFKHKMVYEDTLTLFPFSGETVFMSMENPGLWILGCHNSDFRNRGMTALLKVSSCDKNT |
| | GDYYEDSYEDISAYLLSKNNAIEPRSFSQNPPVLKRHQREITRTTLQSDQEEIDYDDTISVEMKK |
| | EDFDIYDEDENQSPRSFQKKTRHYFIAAVERLWDYGMSSSPHVLRNRAQSGSVPQFKKVVFQE |
| | FTDGSFTQPLYRGELNEHLGLLGPYIRAEVEDNIMVTFRNQASRPYSFYSSLISYEEDQRQGAEP |
| | RKNFVKPNETKTYFWKVQHHMAPTKDEFDCKAWAYFSDVDLEKDVHSGLIGPLLVCHTNTL |
| | NPAHGRQVTVQEFALFFTIFDETKSWYFTENMERNCRAPCNIQMEDPTFKENYRFHAINGYIM |
| | DTLPGLVMAQDQRIRWYLLSMGSNENIHSIHFSGHVFTVRKKEEYKMALYNLYPGVFETVEM |
| | LPSKAGIWRVECLIGEHLHAGMSTLFLVYSNKCQTPLGMASGHIRDFQITASGQYGQWAPKLA |
| | RLHYSGSINAWSTKEPFSWIKVDLLAPMIIHGIKTQGARQKFSSLYISQFIIMYSLDGKKWQTYR |
| | GNSTGTLMVFFGNVDSSGIKHNIFNPPIIARYIRLHPTHYSIRSTLRMELMGCDLNSCSMPLGME |
| | SKAISDAQITASSYFTNMFATWSPSKARLHLQGRSNAWRPQVNNPKEWLQVDFQKTMKVTGV |
| | TTQGVKSLLTSMYVKEFLISSSQDGHQWTLFFQNGKVKVFQGNQDSFTPVVNSLDPPLLTRYL |
| | RIHPQSWVHQIALRMEVLGCEAQDLY |
| FVIII | ATRRYYLGAVELSWDYMQSDLGELPVDARFPPRVPKSFPFNTSVVYKKTLFVEFTDHLFNIAK |
| BDD9- | PRPPWMGLLGPTIQAEVYDTVVITLKNMASHPVSLHAVGVSYWKASEGAEYDDQTSQREKED |
| AE288 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGTSESATPESGPGS |
| | EPATSGSETPGTSESATPESGPGSEPATSGSETPGTSESATPESGPGTSTEPSEGSAPGSPAGSPTS |
| | TEEGTSESATPESGPGSEPATSGSETPGTSESATPESGPGSPAGSPTSTEEGSPAGSPTSTEEGTST |
| | EPSEGSAPGTSESATPESGPGTSESATPESGPGTSESATPESGPGSEPATSGSETPGSEPATSGSET |
| | PGSPAGSPTSTEEGTSTEPSEGSAPGTSTEPSEGSAPGSEPATSGSETPGTSESATPESGPGTSTEP |
| | SEGSAP |
| FVIII | ATRRYYLGAVELSWDYMQSDLGELPVDARFPPRVPKSFPFNTSVVYKKTLFVEFTDHLFNIAK |
| BDD9- | PRPPWMGLLGPTIQAEVYDTVVITLKNMASHPVSLHAVGVSYWKASEGAEYDDQTSQREKED |
| AE864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSPAGSPTSTEEGT |
| | SESATPESGPGTSTEPSEGSAPGSPAGSPTSTEEGTSTEPSEGSAPGTSTEPSEGSAPGTSESATPE |
| | SGPGSEPATSGSETPGSEPATSGSETPGSPAGSPTSTEEGTSESATPESGPGTSTEPSEGSAPGTST |
| | EPSEGSAPGSPAGSPTSTEEGTSTEPSEGSAPGTSTEPSEGSAPGTSESATPESGPGTSTEPSEGSA |
| | PGTSESATPESGPGSEPATSGSETPGTSTEPSEGSAPGTSTEPSEGSAPGTSESATPESGPGTSESA |
| | TPESGPGSPAGSPTSTEEGTSESATPESGPGSEPATSGSETPGTSESATPESGPGTSTEPSEGSAPG |
| | TSTEPSEGSAPGTSTEPSEGSAPGTSTEPSEGSAPGTSTEPSEGSAPGTSTEPSEGSAPGSPAGSPT |
| | STEEGTSTEPSEGSAPGTSESATPESGPGSEPATSGSETPGTSESATPESGPGSEPATSGSETPGTS |
| | ESATPESGPGTSTEPSEGSAPGTSESATPESGPGSPAGSPTSTEEGSPAGSPTSTEEGSPAGSPTST |
| | EEGTSESATPESGPGTSTEPSEGSAPGTSESATPESGPGSEPATSGSETPGTSESATPESGPGSEPA |
| | TSGSETPGTSESATPESGPGTSTEPSEGSAPGSPAGSPTSTEEGTSESATPESGPGSEPATSGSETP |
| | GTSESATPESGPGSPAGSPTSTEEGSPAGSPTSTEEGTSTEPSEGSAPGTSESATPESGPGTSESAT |
| | PESGPGTSESATPESGPGSEPATSGSETPGSEPATSGSETPGSPAGSPTSTEEGTSTEPSEGSAPGT |
| | STEPSEGSAPGSEPATSGSETPGTSESATPESGPGTSTEPSEGSAP |
| FVIII | ATRRYYLGAVELSWDYMQSDLGELPVDARFPPRVPKSFPFNTSVVYKKTLFVEFTDHLFNIAK |
| BDD9- | PRPPWMGLLGPTIQAEVYDTVVITLKNMASHPVSLHAVGVSYWKASEGAEYDDQTSQREKED |
| AG288_3 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSSPSASTGTGPG |
| | SSPSASTGTGPGTPGSGTASSSPGSSTPSGATGSPGSSPSASTGTGPGASPGTSSTGSPGTPGSGT |
| | ASSSPGSSTPSGATGSPGTPGSGTASSSPGASPGTSSTGSPGASPGTSSTGSPGTPGSGTASSSPGS |
| | STPSGATGSPGASPGTSSTGSPGTPGSGTASSSPGSSTPSGATGSPGSSPSASTGTGPGSSPSASTG |
| | TGPGSSTPSGATGSPGSSTPSGATGSPGASPGTSSTGSPGASPGTSSTGSPGASPGTSSTGSPGTP |
| | GSGTASSSP |
| FVIII | ATRRYYLGAVELSWDYMQSDLGELPVDARFPPRVPKSFPFNTSVVYKKTLFVEFTDHLFNIAK |
| BDD9- | PRPPWMGLLGPTIQAEVYDTVVITLKNMASHPVSLHAVGVSYWKASEGAEYDDQTSQREKED |
| AG288_2 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGAGSPGAETAPGAS |
| | PGTSSTGSPGASPGTSSTGSPGTPGSGTASSSPGSSTPSGATGSPGTPGSGTASSSPGSSTPSGATG |
| | SPGTPGSGTASSSPGSSTPSGATGSPGSSTPSGATGSPGSSPSASTGTGPGSSPSASTGTGPGASPG |
| | TSSTGSPGTPGSGTASSSPGSSTPSGATGSPGSSPSASTGTGPGSSPSASTGTGPGASPGTSSTGSP |
| | GASPGTSSTGSPGSSTPSGATGSPGSSPSASTGTGPGASPGTSSTGSPGSSPSASTGTGPGTPGSG |
| | TASSSPGSSTPSGATGS |
| FVIII | ATRRYYLGAVELSWDYMQSDLGELPVDARFPPRVPKSFPFNTSVVYKKTLFVEFTDHLFNIAK |
| BDD9- | PRPPWMGLLGPTIQAEVYDTVVITLKNMASHPVSLHAVGVSYWKASEGAEYDDQTSQREKED |
| AG864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQREITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSSPSASTGTGPG |
| | SSPSASTGTGPGTPGSGTASSSPGSSTPSGATGSPGSSPSASTGTGPGASPGTSSTGSPGTPGSGT |
| | ASSSPGSSTPSGATGSPGTPGSGTASSSPGASPGTSSTGSPGASPGTSSTGSPGTPGSGTASSSPGS |
| | STPSGATGSPGASPGTSSTGSPGTPGSGTASSSPGSSTPSGATGSPGSSPSASTGTGPGSSPSASTG |
| | TGPGSSTPSGATGSPGSSTPSGATGSPGASPGTSSTGSPGASPGTSSTGSPGASPGTSSTGSPGTP |
| | GSGTASSSP |
| FVIII | ATRRYYLGAVELSWDYMQSDLGELPVDARFPPRVPKSFPFNTSVVYKKTLFVEFTDHLFNIAK |
| BDD10- | PRPPWMGLLGPTIQAEVYDTVVITLKNMASHPVSLHAVGVSYWKASEGAEYDDQTSQREKED |
| AG288_2 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQAEITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGAGSPGAETAPGAS |
| | PGTSSTGSPGASPGTSSTGSPGTPGSGTASSSPGSSTPSGATGSPGTPGSGTASSSPGSSTPSGATG |
| | SPGTPGSGTASSSPGSSTPSGATGSPGSSTPSGATGSPGSSPSASTGTGPGSSPSASTGTGPGASPG |
| | TSSTGSPGTPGSGTASSSPGSSTPSGATGSPGSSPSASTGTGPGSSPSASTGTGPGASPGTSSTGSP |
| | GASPGTSSTGSPGSSTPSGATGSPGSSPSASTGTGPGASPGTSSTGSPGSSPSASTGTGPGTPGSG |
| | TASSSPGSSTPSGATGS |
| FVIII | ATRRYYLGAVELSWDYMQSDLGELPVDARFPPRVPKSFPFNTSVVYKKTLFVEFTDHLFNIAK |
| BDD10- | PRPPWMGLLGPTIQAEVYDTVVITLKNMASHPVSLHAVGVSYWKASEGAEYDDQTSQREKED |
| AG864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQAEITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLY |
| FVIII | ATRRYYLGAVELSWDYMQSDLGELPVDARFPPRVPKSFPFNTSVVYKKTLFVEFTDHLFNIAK |
| BDD10- | PRPPWMGLLGPTIQAEVYDTVVITLKNMASHPVSLHAVGVSYWKASEGAEYDDQTSQREKED |
| AE288 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQAEITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGTSESATPESGPGS |
| | EPATSGSETPGTSESATPESGPGSEPATSGSETPGTSESATPESGPGTSTEPSEGSAPGSPAGSPTS |
| | TEEGTSESATPESGPGSEPATSGSETPGTSESATPESGPGSPAGSPTSTEEGSPAGSPTSTEEGTST |
| | EPSEGSAPGTSESATPESGPGTSESATPESGPGTSESATPESGPGSEPATSGSETPGSEPATSGSET |
| | PGSPAGSPTSTEEGTSTEPSEGSAPGTSTEPSEGSAPGSEPATSGSETPGTSESATPESGPGTSTEP |
| | SEGSAP |
| FVIII | ATRRYYLGAVELSWDYMQSDLGELPVDARFPPRVPKSFPFNTSVVYKKTLFVEFTDHLFNIAK |
| BDD10- | PRPPWMGLLGPTIQAEVYDTVVITLKNMASHPVSLHAVGVSYWKASEGAEYDDQTSQREKED |
| AE864 | DKVFPGGSHTYVWQVLKENGPMASDPLCLTYSYLSHVDLVKDLNSGLIGALLVCREGSLAKE |
| | KTQTLHKFILLFAVFDEGKSWHSETKNSLMQDRDAASARAWPKMHTVNGYVNRSLPGLIGCH |
| | RKSVYWHVIGMGTTPEVHSIFLEGHTFLVRNHRQASLEISPITFLTAQTLLMDLGQFLLFCHISS |
| | HQHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETFK |
| | TREAIQHESGILGPLLYGEVGDTLLIIFKNQASRPYNIYPHGITDVRPLYSRRLPKGVKHLKDFPI |
| | LPGEIFKYKWTVTVEDGPTKSDPRCLTRYYSSFVNMERDLASGLIGPLLICYKESVDQRGNQIM |
| | SDKRNVILFSVFDENRSWYLTENIQRFLPNPAGVQLEDPEFQASNIMHSINGYVFDSLQLSVCL |
| | HEVAYWYILSIGAQTDFLSVFFSGYTFKHKMVYEDTLTLFPFSGETVFMSMENPGLWILGCHN |
| | SDFRNRGMTALLKVSSCDKNTGDYYEDSYEDISAYLLSKNNAIEPRSFSQNPPVLKRHQAEITR |
| | TTLQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPTKDEFDCKAWAYFSDV |
| | DLEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKFS |
| | SLYISQFIIMYSLDGKKWQTYRGNSTGTLMVFFGNVDSSGIKHNIFNPPIIARYIRLHPTHYSIRS |
| | TLRMELMGCDLNSCSMPLGMESKAISDAQITASSYFTNMFATWSPSKARLHLQGRSNAWRPQ |
| | VNNPKEWLQVDFQKTMKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQDLYGGSPAGSPTSTEEGT |
| | SESATPESGPGTSTEPSEGSAPGSPAGSPTSTEEGTSTEPSEGSAPGTSTEPSEGSAPGTSESATPE |
| | SGPGSEPATSGSETPGSEPATSGSETPGSPAGSPTSTEEGTSESATPESGPGTSTEPSEGSAPGTST |
| | EPSEGSAPGSPAGSPTSTEEGTSTEPSEGSAPGTSTEPSEGSAPGTSESATPESGPGTSTEPSEGSA |
| | PGTSESATPESGPGSEPATSGSETPGTSTEPSEGSAPGTSTEPSEGSAPGTSESATPESGPGTSESA |
| | TPESGPGSPAGSPTSTEEGTSESATPESGPGSEPATSGSETPGTSESATPESGPGTSTEPSEGSAPG |
| | TSTEPSEGSAPGTSTEPSEGSAPGTSTEPSEGSAPGTSTEPSEGSAPGTSTEPSEGSAPGSPAGSPT |
| | STEEGTSTEPSEGSAPGTSESATPESGPGSEPATSGSETPGTSESATPESGPGSEPATSGSETPGTS |
| | ESATPESGPGTSTEPSEGSAPGTSESATPESGPGSPAGSPTSTEEGSPAGSPTSTEEGSPAGSPTST |
| | EEGTSESATPESGPGTSTEPSEGSAPGTSESATPESGPGSEPATSGSETPGTSESATPESGPGSEPA |
| | TSGSETPGTSESATPESGPGTSTEPSEGSAPGSPAGSPTSTEEGTSESATPESGPGSEPATSGSETP |
| | GTSESATPESGPGSPAGSPTSTEEGSPAGSPTSTEEGTSTEPSEGSAPGTSESATPESGPGTSESAT |
| | PESGPGTSESATPESGPGSEPATSGSETPGSEPATSGSETPGSPAGSPTSTEEGTSTEPSEGSAPGT |
| | STEPSEGSAPGSEPATSGSETPGTSESATPESGPGTSTEPSEGSAP |

*Sequence name reflects N- to C-terminus configuration of the coagulation factor and XTEN components

TABLE 29
Exemplary CFXTEN comprising FVIII and internal/external XTEN sequences

| CFXTEN | |
|---|---|
| Name* | Amino Acid Sequence |
| FVIII BDD2 | ATRRYYLGAVELSWDYMQSDLGELPVDARFPPRVPKSFPFNTSVVYKKTLFVEFTVHLFNIA |
| (A1-K127- | KPRPPWMGLLGPTIQAEVYDTVVITLKNMASHPVSLHAVGVSYWKASEGAEYDDQTSQRE |
| AE144- | KEDDKGGSEPATSGSETPGTSESATPESGPGSEPATSGSETPGSPAGSPTSTEEGTSTEPSEGSA |
| V128-N745- | PGSEPATSGSETPGSEPATSGSETPGSEPATSGSETPGTSTEPSEGSAPGTSESATPESGPGSEPA |
| AE288- | TSGSETPGTSTEPSEGSAPGVFPGGSHTYVWQVLKENGPMASDPLCLTYSYLSHVDLVKDLN |
| P1640- | SGLIGALLVCREGSLAKEKTQTLHKFILLFAVFDEGKSWHSETKNSLMQDRDAASARAWPK |
| Y2332) | MHTVNGYVNRSLPGLIGCHRKSVYWHVIGMGTTPEVHSIFLEGHTFLVRNHRQASLEISPITF |
| | LTAQTLLMDLGQFLLFCHISSHQHDGMEAYVKVDSCPEEPQLRMKNNEEAEDYDDDLTDSE |
| | MDVVRFDDDNSPSFIQIRSVAKKHPKTWVHYIAAEEEDWDYAPLVLAPDDRSYKSQYLNNG |
| | PQRIGRKYKKVRFMAYTDETFKTREAIQHESGILGPLLYGEVGDTLLIIFKNQASRPYNIYPH |
| | GITDVRPLYSRRLPKGVKHLKDFPILPGEIFKYKWTVTVEDGPTKSDPRCLTRYYSSFVNMER |
| | DLASGLIGPLLICYKESVDQRGNQIMSDKRNVILFSVFDENRSWYLTENIQRFLPNPAGVQLE |
| | DPEFQASNIMHSINGYVFDSLQLSVCLHEVAYWYILSIGAQTDFLSVFFSGYTFKHKMVYED |
| | TLTLFPFSGETVFMSMENPGLWILGCHNSDFRNRGMTALLKVSSCDKNTGDYYEDSYEDISA |
| | YLLSKNNAIEPRSFSQNGGTSESATPESGPGSEPATSGSETPGTSESATPESGPGSEPATSGSET |
| | PGTSESATPESGPGTSTEPSEGSAPGSPAGSPTSTEEGTSESATPESGPGSEPATSGSETPGTSES |
| | ATPESGPGSPAGSPTSTEEGSPAGSPTSTEEGTSTEPSEGSAPGTSESATPESGPGTSESATPESG |
| | PGTSESATPESGPGSEPATSGSETPGSEPATSGSETPGSPAGSPTSTEEGTSTEPSEGSAPGTSTE |
| | PSEGSAPGSEPATSGSETPGTSESATPESGPGTSTEPSEGSAPGPPVLKRHQREITRTTLQSDQE |
| | EIDYDDTISVEMKKEDFDIYDEDENQSPRSFQKKTRHYFIAAVERLWDYGMSSSPHVLRNRA |
| | QSGSVPQFKKVVFQEFTDGSFTQPLYRGELNEHLGLLGPYIRAEVEDNIMVTFRNQASRPYSF |
| | YSSLISYEEDQRQGAEPRKNFVKPNETKTYFWKVQHHMAPTKDEFDCKAWAYFSDVDLEK |
| | DVHSGLIGPLLVCHTNTLNPAHGRQVTVQEFALFFTIFDETKSWYFTENMERNCRAPCNIQM |
| | EDPTFKENYRFHAINGYIMDTLPGLVMAQDQRIRWYLLSMGSNENIHSIHFSGHVFTVRKKE |
| | EYKMALYNLYPGVFETVEMLPSKAGIWRVECLIGEHLHAGMSTLFLVYSNKCQTPLGMASG |
| | HIRDFQITASGQYGQWAPKLARLHYSGSINAWSTKEPFSWIKVDLLAPMIIHGIKTQGARQKF |
| | SSLYISQFIIMYSLDGKKWQTYRGNSTGTLMVFFGNVDSSGIKHNIFNPPIIARYIRLHPTHYSI |
| | RSTLRMELMGCDLNSCSMPLGMESKAISDAQITASSYFTNMFATWSPSKARLHLQGRSNAW |
| | RPQVNNPKEWLQVDFQKTMKVTGVTTQGVKSLLTSMYVKEFLISSSQDGHQWTLFFQNGK |
| | VKVFQGNQDSFTPVVNSLDPPLLTRYLRIHPQSWVHQIALRMEVLGCEAQDLY |
| FVIII BDD2 | ATRRYYLGAVELSWDYMQSDLGELPVDARFPPRVPKSFPFNTSVVYKKTLFVEFTVHLFNIA |
| (A1-A375- | KPRPPWMGLLGPTIQAEVYDTVVITLKNMASHPVSLHAVGVSYWKASEGAEYDDQTSQRE |
| AE576- | KEDDKVFPGGSHTYVWQVLKENGPMASDPLCLTYSYLSHVDLVKDLNSGLIGALLVCREGS |
| K376-N745- | LAKEKTQTLHKFILLFAVFDEGKSWHSETKNSLMQDRDAASARAWPKMHTVNGYVNRSLP |
| AE144- | GLIGCHRKSVYWHVIGMGTTPEVHSIFLEGHTFLVRNHRQASLEISPITFLTAQTLLMDLGQF |
| P1640- | LLFCHISSHQHDGMEAYVKVDSCPEEPQLRMKNNEEAEDYDDDLTDSEMDVVRFDDDNSP |
| Y2332) | SFIQIRSVAGGSPAGSPTSTEEGTSESATPESGPGTSTEPSEGSAPGSPAGSPTSTEEGTSTEPSE |
| | GSAPGTSTEPSEGSAPGTSESATPESGPGSEPATSGSETPGSEPATSGSETPGSPAGSPTSTEEG |
| | TSESATPESGPGTSTEPSEGSAPGTSTEPSEGSAPGSPAGSPTSTEEGTSTEPSEGSAPGTSTEPS |
| | EGSAPGTSESATPESGPGTSTEPSEGSAPGTSESATPESGPGSEPATSGSETPGTSTEPSEGSAP |
| | GTSTEPSEGSAPGTSESATPESGPGTSESATPESGPGSPAGSPTSTEEGTSESATPESGPGSEPAT |
| | SGSETPGTSESATPESGPGTSTEPSEGSAPGTSTEPSEGSAPGTSTEPSEGSAPGTSTEPSEGSAP |
| | GTSTEPSEGSAPGTSTEPSEGSAPGSPAGSPTSTEEGTSTEPSEGSAPGTSESATPESGPGSEPAT |
| | SGSETPGTSESATPESGPGSEPATSGSETPGTSESATPESGPGTSTEPSEGSAPGTSESATPESGP |
| | GSPAGSPTSTEEGSPAGSPTSTEEGSPAGSPTSTEEGTSESATPESGPGTSTEPSEGSAPGKKHP |
| | KTWVHYIAAEEEDWDYAPLVLAPDDRSYKSQYLNNGPQRIGRKYKKVRFMAYTDETFKTR |
| | EAIQHESGILGPLLYGEVGDTLLIIFKNQASRPYNIYPHGITDVRPLYSRRLPKGVKHLKDFPIL |
| | PGEIFKYKWTVTVEDGPTKSDPRCLTRYYSSFVNMERDLASGLIGPLLICYKESVDQRGNQI |
| | MSDKRNVILFSVFDENRSWYLTENIQRFLPNPAGVQLEDPEFQASNIMHSINGYVFDSLQLSV |
| | CLHEVAYWYILSIGAQTDFLSVFFSGYTFKHKMVYEDTLTLFPFSGETVFMSMENPGLWILG |
| | CHNSDFRNRGMTALLKVSSCDKNTGDYYEDSYEDISAYLLSKNNAIEPRSFSQNGGSEPATS |
| | GSETPGTSESATPESGPGSEPATSGSETPGSPAGSPTSTEEGTSTEPSEGSAPGSEPATSGSETPG |
| | SEPATSGSETPGSEPATSGSETPGTSTEPSEGSAPGTSESATPESGPGSEPATSGSETPGTSTEPS |
| | EGSAPGPPVLKRHQREITRTTLQSDQEEIDYDDTISVEMKKEDFDIYDEDENQSPRSFQKKTR |
| | HYFIAAVERLWDYGMSSSPHVLRNRAQSGSVPQFKKVVFQEFTDGSFTQPLYRGELNEHLG |
| | LLGPYIRAEVEDNIMVTFRNQASRPYSFYSSLISYEEDQRQGAEPRKNFVKPNETKTYFWKV |
| | QHHMAPTKDEFDCKAWAYFSDVDLEKDVHSGLIGPLLVCHTNTLNPAHGRQVTVQEFALF |
| | FTIFDETKSWYFTENMERNCRAPCNIQMEDPTFKENYRFHAINGYIMDTLPGLVMAQDQRIR |
| | WYLLSMGSNENIHSIHFSGHVFTVRKKEEYKMALYNLYPGVFETVEMLPSKAGIWRVECLI |
| | GEHLHAGMSTLFLVYSNKCQTPLGMASGHIRDFQITASGQYGQWAPKLARLHYSGSINAWS |
| | TKEPFSWIKVDLLAPMIIHGIKTQGARQKFSSLYISQFIIMYSLDGKKWQTYRGNSTGTLMVF |
| | FGNVDSSGIKHNIFNPPIIARYIRLHPTHYSIRSTLRMELMGCDLNSCSMPLGMESKAISDAQIT |
| | ASSYFTNMFATWSPSKARLHLQGRSNAWRPQVNNPKEWLQVDFQKTMKVTGVTTQGVKS |
| | LLTSMYVKEFLISSSQDGHQWTLFFQNGKVKVFQGNQDSFTPVVNSLDPPLLTRYLRIHPQS |
| | WVHQIALRMEVLGCEAQDLY |
| FVIII BDD2 | ATRRYYLGAVELSWDYMQSDLGELPVDARFPPRVPKSFPFNTSVVYKKTLFVEFTVHLFNIA |
| (A1-Y1792- | KPRPPWMGLLGPTIQAEVYDTVVITLKNMASHPVSLHAVGVSYWKASEGAEYDDQTSQRE |
| AF144- | KEDDKVFPGGSHTYVWQVLKENGPMASDPLCLTYSYLSHVDLVKDLNSGLIGALLVCREGS |
| E1793- | LAKEKTQTLHKFILLFAVFDEGKSWHSETKNSLMQDRDAASARAWPKMHTVNGYVNRSLP |
| Y2332- | GLIGCHRKSVYWHVIGMGTTPEVHSIFLEGHTFLVRNHRQASLEISPITFLTAQTLLMDLGQF |
| AE864) | LLFCHISSHQHDGMEAYVKVDSCPEEPQLRMKNNEEAEDYDDDLTDSEMDVVRFDDDNSP |
| | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSQNPPVLKRHQREITRTTLQSDQEEIDYDDTISVEMKKEDFDIYDEDENQSPRSFQKKTRH |
| | YFIAAVERLWDYGMSSSPHVLRNRAQSGSVPQFKKVVFQEFTDGSFTQPLYRGELNEHLGL |
| | LGPYIRAEVEDNIMVTFRNQASRPYSFYSSLISYGGTSTPESGSASPGTSPSGESSTAPGTSPSG |
| | ESSTAPGSTSSTAESPGPGSTSESPSGTAPGSTSSTAESPGPGTSPSGESSTAPGTSTPESGSASP |
| | GSTSSTAESPGPGTSPSGESSTAPGTSPSGESSTAPGTSPSGESSTAPGEEDQRQGAEPRKNFV |
| | KPNETKTYFWKVQHHMAPTKDEFDCKAWAYFSDVDLEKDVHSGLIGPLLVCHTNTLNPAH |
| | GRQVTVQEFALFFTIFDETKSWYFTENMERNCRAPCNIQMEDPTFKENYRFHAINGYIMDTL |
| | PGLVMAQDQRIRWYLLSMGSNENIHSIHFSGHVFTVRKKEEYKMALYNLYPGVFETVEMLP |
| | SKAGIWRVECLIGEHLHAGMSTLFLVYSNKCQTPLGMASGHIRDFQITASGQYGQWAPKLA |
| | RLHYSGSINAWSTKEPFSWIKVDLLAPMIIHGIKTQGARQKFSSLYISQFIIMYSLDGKKWQT |
| | YRGNSTGTLMVFFGNVDSSGIKHNIFNPPIIARYIRLHPTHYSIRSTLRMELMGCDLNSCSMPL |
| | GMESKAISDAQITASSYFTNMFATWSPSKARLHLQGRSNAWRPQVNNPKEWLQVDFQKTM |
| | KVTGVTTQGVKSLLTSMYVKEFLISSSQDGHQWTLFFQNGKVKVFQGNQDSFTPVVNSLDP |
| | PLLTRYLRIHPQSWVHQIALRMEVLGCEAQDLYGGSPAGSPTSTEEGTSESATPESGPGTSTE |
| | PSEGSAPGSPAGSPTSTEEGTSTEPSEGSAPGTSTEPSEGSAPGTSESATPESGPGSEPATSGSET |
| | PGSEPATSGSETPGSPAGSPTSTEEGTSESATPESGPGTSTEPSEGSAPGTSTEPSEGSAPGSPA |
| | GSPTSTEEGTSTEPSEGSAPGTSTEPSEGSAPGTSESATPESGPGTSTEPSEGSAPGTSESATPES |
| | GPGSEPATSGSETPGTSTEPSEGSAPGTSTEPSEGSAPGTSESATPESGPGTSESATPESGPGSP |
| | AGSPTSTEEGTSESATPESGPGSEPATSGSETPGTSESATPESGPGTSTEPSEGSAPGTSTEPSEG |
| | SAPGTSTEPSEGSAPGTSTEPSEGSAPGTSTEPSEGSAPGTSTEPSEGSAPGSPAGSPTSTEEGTS |
| | TEPSEGSAPGTSESATPESGPGSEPATSGSETPGTSESATPESGPGSEPATSGSETPGTSESATPE |
| | SGPGTSTEPSEGSAPGTSESATPESGPGSPAGSPTSTEEGSPAGSPTSTEEGSPAGSPTSTEEGTS |
| | ESATPESGPGTSTEPSEGSAPGTSESATPESGPGSEPATSGSETPGTSESATPESGPGSEPATSGS |
| | ETPGTSESATPESGPGTSTEPSEGSAPGSPAGSPTSTEEGTSESATPESGPGSEPATSGSETPGTS |
| | ESATPESGPGSPAGSPTSTEEGSPAGSPTSTEEGTSTEPSEGSAPGTSESATPESGPGTSESATPE |
| | SGPGTSESATPESGPGSEPATSGSETPGSEPATSGSETPGSPAGSPTSTEEGTSTEPSEGSAPGTS |
| | TEPSEGSAPGSEPATSGSETPGTSESATPESGPGTSTEPSEGSAP |
| FVIII BDD2 | ATRRYYLGAVELSWDYMQSDLGELPVDARFPPRVPKSFPFNTSVVYKKTLFVEFTVHLFNIA |
| (A1-Y2043- | KPRPPWMGLLGPTIQAEVYDTVVITLKNMASHPVSLHAVGVSYWKASEGAEYDDQTSQRE |
| AG144- | KEDDKVFPGGSHTYVWQVLKENGPMASDPLCLTYSYLSHVDLVKDLNSGLIGALLVCREGS |
| G2044- | LAKEKTQTLHKFILLFAVFDEGKSWHSETKNSLMQDRDAASARAWPKMHTVNGYVNRSLP |
| Q2222- | GLIGCHRKSVYWHVIGMGTTPEVHSIFLEGHTFLVRNHRQASLEISPITFLTAQTLLMDLGQF |
| AG864- | LLFCHISSHQHDGMEAYVKVDSCPEEPQLRMKNNEEAEDYDDDLTDSEMDVVRFDDDNSP |
| V2223- | SFIQIRSVAKKHPKTWVHYIAAEEEDWDYAPLVLAPDDRSYKSQYLNNGPQRIGRKYKKVR |
| Y2332) | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSQNPPVLKRHQREITRTTLQSDQEEIDYDDTISVEMKKEDFDIYDEDENQSPRSFQKKTRH |
| | YFIAAVERLWDYGMSSSPHVLRNRAQSGSVPQFKKVVFQEFTDGSFTQPLYRGELNEHLGL |
| | LGPYIRAEVEDNIMVTFRNQASRPYSFYSSLISYEEDQRQGAEPRKNFVKPNETKTYFWKVQ |
| | HHMAPTKDEFDCKAWAYFSDVDLEKDVHSGLIGPLLVCHTNTLNPAHGRQVTVQEFALFFT |
| | IFDETKSWYFTENMERNCRAPCNIQMEDPTFKENYRFHAINGYIMDTLPGLVMAQDQRIRW |
| | YLLSMGSNENIHSIHFSGHVFTVRKKEEYKMALYNLYPGVFETVEMLPSKAGIWRVECLIGE |
| | HLHAGMSTLFLVYSNKCQTPLGMASGHIRDFQITASGQYGPGSSPSASTGTGPGSSPSASTGT |
| | GPGTPGSGTASSSPGSSTPSGATGSPGSSPSASTGTGPGASPGTSSTGSPGTPGSGTASSSPGSS |
| | TPSGATGSPGTPGSGTASSSPGASPGTSSTGSPGASPGTSSTGSPGTPGSGTASSSGGQWAPKL |
| | ARLHYSGSINAWSTKEPFSWIKVDLLAPMIIHGIKTQGARQKFSSLYISQFIIMYSLDGKKWQ |
| | TYRGNSTGTLMVFFGNVDSSGIKHNIFNPPIIARYIRLHPTHYSIRSTLRMELMGCDLNSCSMP |
| | LGMESKAISDAQITASSYFTNMFATWSPSKARLHLQGRSNAWRPQGGASPGTSSTGSPGSSP |
| | SASTGTGPGSSPSASTGTGPGTPGSGTASSSPGSSTPSGATGSPGSSPSASTGTGPGASPGTSST |
| | GSPGTPGSGTASSSPGSSTPSGATGSPGTPGSGTASSSPGASPGTSSTGSPGASPGTSSTGSPGT |
| | PGSGTASSSPGSSTPSGATGSPGASPGTSSTGSPGTPGSGTASSSPGSSTPSGATGSPGSSPSAST |
| | GTGPGSSPSASTGTGPGSSTPSGATGSPGSSTPSGATGSPGASPGTSSTGSPGASPGTSSTGSPG |
| | ASPGTSSTGSPGTPGSGTASSSPGASPGTSSTGSPGASPGTSSTGSPGASPGTSSTGSPGSSPSAS |
| | TGTGPGTPGSGTASSSPGASPGTSSTGSPGASPGTSSTGSPGASPGTSSTGSPGSSTPSGATGSP |
| | GSSTPSGATGSPGASPGTSSTGSPGTPGSGTASSSPGSSTPSGATGSPGSSTPSGATGSPGSSTP |
| | SGATGSPGSSPSASTGTGPGASPGTSSTGSPGASPGTSSTGSPGTPGSGTASSSPGASPGTSSTG |
| | SPGASPGTSSTGSPGASPGTSSTGSPGASPGTSSTGSPGTPGSGTASSSPGSSTPSGATGSPGTP |
| | GSGTASSSPGSSTPSGATGSPGTPGSGTASSSPGSSTPSGATGSPGSSTPSGATGSPGSSPSAST |
| | GTGPGSSPSASTGTGPGASPGTSSTGSPGTPGSGTASSSPGSSTPSGATGSPGSSPSASTGTGPG |
| | SSPSASTGTGPGASPGTSSTGSPGASPGTSSTGSPGSSTPSGATGSPGSSPSASTGTGPGASPGT |
| | SSTGSPGSSPSASTGTGPGTPGSGTASSSPGSSTPSGATGSPGSSTPSGATGSPGASPGTSSTGS |
| | PGVNNPKEWLQVDFQKTMKVTGVTTQGVKSLLTSMYVKEFLISSSQDGHQWTLFFQNGKV |
| | KVFQGNQDSFTPVVNSLDPPLLTRYLRIHPQSWVHQIALRMEVLGCEAQDLY |
| FVIII BDD2 | ATRRYYLGAVELSWDYMQSDLGELPVDARFPPRVPKSFPFNTSVVYKKTLFVEFTVHLFNI |
| (A1-G1799- | AKPRPPWMGLLGPTIQAEVYDTVVITLKNMASHPVSLHAVGVSYWKASEGAEYDDQTSQ |
| AE144- | REKEDDKVFPGGSHTYVWQVLKENGPMASDPLCLTYSYLSHVDLVKDLNSGLIGALLVCR |
| A1800- | EGSLAKEKTQTLHKFILLFAVFDEGKSWHSETKNSLMQDRDAASARAWPKMHTVNGYVN |
| F2093- | RSLPGLIGCHRKSVYWHVIGMGTTPEVHSIFLEGHTFLVRNHRQASLEISPITFLTAQTLLMD |
| AE42- | LGQFLLFCHISSHQHDGMEAYVKVDSCPEEPQLRMKNNEEAEDYDDDLTDSEMDVVRFDD |
| S2094- | DNSPSFIQIRSVAKKHPKTWVHYIAAEEEDWDYAPLVLAPDDRSYKSQYLNNGPQRIGRKY |
| V2223- | KKVRFMAYTDETFKTREAIQHESGILGPLLYGEVGDTLLIIFKNQASRPYNIYPHGITDVRPL |
| AE42- | YSRRLPKGVKHLKDFPILPGEIFKYKWTVTVEDGPTKSDPRCLTRYYSSFVNMERDLASGLI |
| N2224- | GPLLICYKESVDQRGNQIMSDKRNVILFSVFDENRSWYLTENIQRFLPNPAGVQLEDPEFQA |
| AE42- | SNIMHSINGYVFDSLQLSVCLHEVAYWYILSIGAQTDFLSVFFSGYTFKHKMVYEDTLTLFP |
| N2225- | FSGETVFMSMENPGLWILGCHNSDFRNRGMTALLKVSSCDKNTGDYYEDSYEDISAYLLS |
| G2278- | KNNAIEPRSFSQNPPVLKRHQREITRTTLQSDQEEIDYDDTISVEMKKEDFDIYDEDENQSPR |
| AE42- | SFQKKTRHYFIAAVERLWDYGMSSSPHVLRNRAQSGSVPQFKKVVFQEFTDGSFTQPLYRG |
| K2279- | ELNEHLGLLGPYIRAEVEDNIMVTFRNQASRPYSFYSSLISYEEDQRQGGGSEPATSGSETPG |
| Y2332) | TSESATPESGPGSEPATSGSETPGSPAGSPTSTEEGTSTEPSEGSAPGSEPATSGSETPGSEPAT |
| | SGSETPGSEPATSGSETPGTSTEPSEGSAPGTSESATPESGPGSEPATSGSETPGTSTEPSEGSA |
| | PGAEPRKNFVKPNETKTYFWKVQHHMAPTKDEFDCKAWAYFSDVDLEKDVHSGLIGPLL |
| | VCHTNTLNPAHGRQVTVQEFALFFTIFDETKSWYFTENMERNCRAPCNIQMEDPTFKENYR |
| | FHAINGYIMDTLPGLVMAQDQRIRWYLLSMGSNENIHSIHFSGHVFTVRKKEEYKMALYNL |
| | YPGVFETVEMLPSKAGIWRVECLIGEHLHAGMSTLFLVYSNKCQTPLGMASGHIRDFQITA |
| | SGQYGQWAPKLARLHYSGSINAWSTKEPFSWIKVDLLAPMIIHGIKTQGARQKFGSEPATS |
| | GSETPGTSESATPESGPGTSTEPSEGSAPGSPAGSPGSSLYISQFIIMYSLDGKKWQTYRGNST |
| | GTLMVFFGNVDSSGIKHNIFNPPIIARYIRLHPTHYSIRSTLRMELMGCDLNSCSMPLGMESK |
| | AISDAQITASSYFTNMFATWSPSKARLHLQGRSNAWRPQVGPAGSPTSTEEGTSTEPSEGSA |
| | PGTSESATPESGPGSEPATSGGNNPKEWLQVDFQKTMKVTGVTTQGVKSLLTSMYVKEFLI |
| | SSSQDGHQWTLFFQNGGTEPSEGSAPGSPAGSPTSTEEGTSESATPESGPGSEPATSGSKVKV |
| | FQGNQDSFTPVVNSLDPPLLTRYLRIHPQSWVHQIALRMEVLGCEAQDLY |
| FVIII BDD2 | ATRRYYLGAVELSWDYMQSDLGELPVDARGPGSSPSASTGTGPGSSPSASTGTGPGTPGSGT |
| (A1-R28- | ASSSPGSSTPSGATGSPGSSPSASTGTGPGASPGTSSTGSPGTPGSGTASSSPGSSTPSGATGSP |
| AG144-F29- | GTPGSGTASSSPGASPGTSSTGSPGASPGTSSTGSPGTPGSGTASSSGFPPRVPKSFPFNTSVVY |
| G244- | KKTLFVEFTVHLFNIAKPRPPWMGLLGPTIQAEVYDTVVITLKNMASHPVSLHAVGVSYWK |
| AG288- | ASEGAEYDDQTSQREKEDDKVFPGGSHTYVWQVLKENGPMASDPLCLTYSYLSHVDLVKD |
| L245- | LNSGLIGALLVCREGSLAKEKTQTLHKFILLFAVFDEGKSWHSETKNSLMQDRDAASARAW |
| R2090- | PKMHTVNGYVNRSLPGGPGASPGTSSTGSPGASPGTSSTGSPGTPGSGTASSSPGSSTPSGAT |
| AG576- | GSPGTPGSGTASSSPGSSTPSGATGSPGTPGSGTASSSPGSSTPSGATGSPGSSTPSGATGSPGS |
| Q2091- | SPSASTGTGPGSSPSASTGTGPGASPGTSSTGSPGTPGSGTASSSPGSSTPSGATGSPGSSPSAS |
| Y2332- | TGTGPGSSPSASTGTGPGASPGTSSTGSPGASPGTSSTGSPGSSTPSGATGSPGSSPSASTGTGP |
| AG864) | GASPGTSSTGSPGSSPSASTGTGPGTPGSGTASSSPGSSTPSGATGSGLIGCHRKSVYWHVIGM |
| | GTTPEVHSIFLEGHTFLVRNHRQASLEISPITFLTAQTLLMDLGQFLLFCHISSHQHDGMEAYV |
| | KVDSCPEEPQLRMKNNEEAEDYDDDLTDSEMDVVRFDDDNSPSFIQIRSVAKKHPKTWVHY |
| | IAAEEEDWDYAPLVLAPDDRSYKSQYLNNGPQRIGRKYKKVRFMAYTDETFKTREAIQHES |
| | GILGPLLYGEVGDTLLIIFKNQASRPYNIYPHGITDVRPLYSRRLPKGVKHLKDFPILPGEIFKY |
| | KWTVTVEDGPTKSDPRCLTRYYSSFVNMERDLASGLIGPLLICYKESVDQRGNQIMSDKRN |
| | VILFSVFDENRSWYLTENIQRFLPNPAGVQLEDPEFQASNIMHSINGYVFDSLQLSVCLHEVA |
| | YWYILSIGAQTDFLSVFFSGYTFKHKMVYEDTLTLFPFSGETVFMSMENPGLWILGCHNSDF |
| | RNRGMTALLKVSSCDKNTGDYYEDSYEDISAYLLSKNNAIEPRSFSQNPPVLKRHQREITRTT |
| | LQSDQEEIDYDDTISVEMKKEDFDIYDEDENQSPRSFQKKTRHYFIAAVERLWDYGMSSSPH |
| | VLRNRAQSGSVPQFKKVVFQEFTDGSFTQPLYRGELNEHLGLLGPYIRAEVEDNIMVTFRNQ |
| | ASRPYSFYSSLISYEEDQRQGAEPRKNFVKPNETKTYFWKVQHHMAPTKDEFDCKAWAYFS |
| | DVDLEKDVHSGLIGPLLVCHTNTLNPAHGRQVTVQEFALFFTIFDETKSWYFTENMERNCRA |
| | PCNIQMEDPTFKENYRFHAGINGYIMDTLPGLVMAQDQRIRWYLLSMGSNENIHSIHFSGHV |
| | FTVRKKEEYKMALYNLYPGVFETVEMLPSKAGIWRVECLIGEHLHAGMSTLFLVYSNKCQT |
| | PLGMASGHIRDFQITASGQYGQWAPKLARLHYSGSINAWSTKEPFSWIKVDLLAPMIIHGIKT |
| | QGARGSPAGSPTSTEEGTSESATPESGPGTSTEPSEGSAPGSPAGSPTSTEEGTSTEPSEGSAPG |
| | TSTEPSEGSAPGTSESATPESGPGSEPATSGSETPGSEPATSGSETPGSPAGSPTSTEEGTSESAT |
| | PESGPGTSTEPSEGSAPGTSTEPSEGSAPGSPAGSPTSTEEGTSTEPSEGSAPGTSTEPSEGSAPG |
| | TSESATPESGPGTSTEPSEGSAPGTSESATPESGPGSEPATSGSETPGTSTEPSEGSAPGTSTEPS |
| | EGSAPGTSESATPESGPGTSESATPESGPGSPAGSPTSTEEGTSESATPESGPGSEPATSGSETP |
| | GTSESATPESGPGTSTEPSEGSAPGTSTEPSEGSAPGTSTEPSEGSAPGTSTEPSEGSAPGTSTEP |
| | SEGSAPGTSTEPSEGSAPGSPAGSPTSTEEGTSTEPSEGSAPGTSESATPESGPGSEPATSGSETP |
| | GTSESATPESGPGSEPATSGSETPGTSESATPESGPGTSTEPSEGSAPGTSESATPESGPGSPAG |
| | SPTSTEEGSPAGSPTSTEEGSPAGSPTSTEEGTSESATPESGPGTSTEPSEGSAPGQKFSSLYISQ |
| | FIIMYSLDGKKWQTYRGNSTGTLMVFFGNVDSSGIKHNIFNPPIIARYIRLHPTHYSIRSTLRM |
| | ELMGCDLNSCSMPLGMESKAISDAQITASSYFTNMFATWSPSKARLHLQGRSNAWRPQVNN |
| | PKEWLQVDFQKTMKVTGVTTQGVKSLLTSMYVKEFLISSSQDGHQWTLFFQNGKVKVFQG |
| | NQDSFTPVVNSLDPPLLTRYLRIHPQSWVHQIALRMEVLGCEAQDLYGGSPAGSPTSTEEGT |
| | SESATPESGPGTSTEPSEGSAPGSPAGSPTSTEEGTSTEPSEGSAPGTSTEPSEGSAPGTSESATP |
| | ESGPGSEPATSGSETPGSEPATSGSETPGSPAGSPTSTEEGTSESATPESGPGTSTEPSEGSAPGT |
| | STEPSEGSAPGSPAGSPTSTEEGTSTEPSEGSAPGTSTEPSEGSAPGTSESATPESGPGTSTEPSE |
| | GSAPGTSESATPESGPGSEPATSGSETPGTSTEPSEGSAPGTSTEPSEGSAPGTSESATPESGPG |
| | TSESATPESGPGSPAGSPTSTEEGTSESATPESGPGSEPATSGSETPGTSESATPESGPGTSTEPS |
| | EGSAPGTSTEPSEGSAPGTSTEPSEGSAPGTSTEPSEGSAPGTSTEPSEGSAPGTSTEPSEGSAP |
| | GSPAGSPTSTEEGTSTEPSEGSAPGTSESATPESGPGSEPATSGSETPGTSESATPESGPGSEPAT |
| | SGSETPGTSESATPESGPGTSTEPSEGSAPGTSESATPESGPGSPAGSPTSTEEGSPAGSPTSTEE |
| | GSPAGSPTSTEEGTSESATPESGPGTSTEPSEGSAPGTSESATPESGPGSEPATSGSETPGTSESA |
| | TPESGPGSEPATSGSETPGTSESATPESGPGTSTEPSEGSAPGSPAGSPTSTEEGTSESATPESGP |
| | GSEPATSGSETPGTSESATPESGPGSPAGSPTSTEEGSPAGSPTSTEEGTSTEPSEGSAPGTSESA |
| | TPESGPGTSESATPESGPGTSESATPESGPGSEPATSGSETPGSEPATSGSETPGSPAGSPTSTEE |
| | GTSTEPSEGSAPGTSTEPSEGSAPGSEPATSGSETPGTSESATPESGPGTSTEPSEGSAP |
| FVIII | ATRRYYLGAVELSWDYMQSDLGELPVDARFPPRVPKSFPFNTSVVYKKTLFVEFTDHLFNIA |
| (A1-T1651- | KPRPPWMGLLGPTIQAEVYDTVVITLKNMASHPVSLHAVGVSYWKASEGAEYDDQTSQRE |
| AG576- | KEDDKVFPGGSHTYVWQVLKENGPMASDPLCLTYSYLSHVDLVKDLNSGLIGALLVCREGS |
| R1652- | LAKEKTQTLHKFILLFAVFDEGKSWHSETKNSLMQDRDAASARAWPKMHTVNGYVNRSLP |
| K1808- | GLIGCHRKSVYWHVIGMGTTPEVHSIFLEGHTFLVRNHRQASLEISPITFLTAQTLLMDLGQF |
| AG144- | LLFCHISSHQHDGMEAYVKVDSCPEEPQLRMKNNEEAEDYDDDLTDSEMDVVRFDDDNSP |
| P1809- | SFIQIRSVAKKHPKTWVHYIAAEEEDWDYAPLVLAPDDRSYKSQYLNNGPQRIGRKYKKVR |
| F2093- | FMAYTDETFKTREAIQHESGILGPLLYGEVGDTLLIIFKNQASRPYNIYPHGITDVRPLYSRRL |
| AG288- | PKGVKHLKDFPILPGEIFKYKWTVTVEDGPTKSDPRCLTRYYSSFVNMERDLASGLIGPLLIC |
| S2094- | YKESVDQRGNQIMSDKRNVILFSVFDENRSWYLTENIQRFLPNPAGVQLEDPEFQASNIMHSI |
| Y2332) | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSQNSRHPSTRQKQFNATTIPENDIEKTDPWFAHRTPMPKIQNVSSSDLLMLLRQSPTPHGL |
| | SLSDLQEAKYETFSDDPSPGAIDSNNSLSEMTHFRPQLHHSGDMVFTPESGLQLRLNEKLGTT |
| | AATELKKLDFKVSSTSNNLISTIPSDNLAAGTDNTSSLGPPSMPVHYDSQLDTTLFGKKSSPLT |
| | ESGGPLSLSEENNDSKLLESGLMNSQESSWGKNVSSTESGRLFKGKRAHGPALLTKDNALFK |
| | VSISLLKTNKTSNNSATNRKTHIDGPSLLIENSPSVWQNILESDTEFKKVTPLIHDRMLMDKN |
| | ATALRLNHMSNKTTSSKNMEMVQQKKEGPIPPDAQNPDMSFFKMLFLPESARWIQRTHGK |
| | NSLNSGQGPSPKQLVSLGPEKSVEGQNFLSEKNKVVVGKGEFTKDVGLKEMVFPSSRNLFLT |
| | NLDNLHENNTHNQEKKIQEEIEKKETLIQENVVLPQIHTVTGTKNFMKNLFLLSTRQNVEGS |
| | YDGAYAPVLQDFRSLNDSTNRTKKHTAHFSKKGEEENLEGLGNQTKQIVEKYACTTRISPNT |
| | SQQNFVTQRSKRALKQFRLPLEETELEKRIIVDDTSTQWSKNMKHLTPSTLTQIDYNEKEKG |
| | AITQSPLSDCLTRSHSIPQANRSPLPIAKVSSFPSIRPIYLTRVLFQDNSSHLPAASYRKKDSGV |
| | QESSHFLQGAKKNNLSLAILTLEMTGDQREVGSLGTSATNSVTYKKVENTVLPKPDLPKTSG |
| | KVELLPKVHIYQKDLFPTETSNGSPGHLDLVEGSLLQGTEGAIKWNEANRPGKVPFLRVATE |
| | SSAKTPSKLLDPLAWDNHYGTQIPKEEWKSQEKSPEKTAFKKKDTILSLNACESNHAIAAINE |
| | GQNKPEIEVTWAKQGRTERLCSQNPPVLKRHQREITGPGTPGSGTASSSPGSSTPSGATGSPG |
| | SSPSASTGTGPGSSPSASTGTGPGSSTPSGATGSPGSSTPSGATGSPGASPGTSSTGSPGASPGT |
| | SSTGSPGASPGTSSTGSPGTPGSGTASSSPGASPGTSSTGSPGASPGTSSTGSPGASPGTSSTGS |
| | PGSSPSASTGTGPGTPGSGTASSSPGASPGTSSTGSPGASPGTSSTGSPGASPGTSSTGSPGSST |
| | PSGATGSPGSSTPSGATGSPGASPGTSSTGSPGTPGSGTASSSPGSSTPSGATGSPGSSTPSGAT |
| | GSPGSSTPSGATGSPGSSPSASTGTGPGASPGTSSTGSPGASPGTSSTGSPGTPGSGTASSSPGA |
| | SPGTSSTGSPGASPGTSSTGSPGASPGTSSTGSPGASPGTSSTGSPGTPGSGTASSSPGSSTPSG |
| | ATGSPGTPGSGTASSSPGSSTPSGATGSPGTPGSGTASSSPGSSTPSGATGSPGSSTPSGATGSP |
| | GSSPSASTGTGPGSSPSASTGTGPGASPGTSSTGSPGTPGSGTASSSPGSSTPSGATGSPGSSPS |
| | ASTGTGPGSSPSASTGTGPGASPGTSSTGSSGRTTLQSDQEEIDYDDTISVEMKKEDFDIYDED |
| | ENQSPRSFQKKTRHYFIAAVERLWDYGMSSSPHVLRNRAQSGSVPQFKKVVFQEFTDGSFT |
| | QPLYRGELNEHLGLLGPYIRAEVEDNIMVTFRNQASRPYSFYSSLISYEEDQRQGAEPRKNFV |
| | KGSGTASSSPGSSTPSGATGSPGTPGSGTASSSPGSSTPSGATGSPGSSTPSGATGSPGSSPSAS |
| | TGTGPGSSPSASTGTGPGASPGTSSTGSPGTPGSGTASSSPGSSTPSGATGSPGSSPSASTGTGP |
| | GSSPSASTGTGPGASPGPNETKTYFWKVQHHMAPTKDEFDCKAWAYFSDVDLEKDVHSGLI |
| | GPLLVCHTNTLNPAHGRQVTVQEFALFFTIFDETKSWYFTENMERNCRAPCNIQMEDPTFKE |
| | NYRFHAINGYIMDTLPGLVMAQDQRIRWYLLSMGSNENIHSIHFSGHVFTVRKKEEYKMAL |
| | YNLYPGVFETVEMLPSKAGIWRVECLIGEHLHAGMSTLFLVYSNKCQTPLGMASGHIRDFQI |
| | TASGQYGQWAPKLARLHYSGSINAWSTKEPFSWIKVDLLAPMIIHGIKTQGARQKFGPGASP |
| | GTSSTGSPGASPGTSSTGSPGTPGSGTASSSPGSSTPSGATGSPGTPGSGTASSSPGSSTPSGAT |
| | GSPGTPGSGTASSSPGSSTPSGATGSPGSSTPSGATGSPGSSPSASTGTGPGSSPSASTGTGPGA |
| | SPGTSSTGSPGTPGSGTASSSPGSSTPSGATGSPGSSPSASTGTGPGSSPSASTGTGPGASPGTS |
| | STGSPGASPGTSSTGSPGSSTPSGATGSPGSSPSASTGTGPGASPGTSSTGSPGSSPSASTGTGP |
| | GTPGSGTASSSPGSSTPSGATGSGSSLYISQFIIMYSLDGKKWQTYRGNSTGTLMVFFGNVDS |
| | SGIKHNIFNPPIIARYIRLHPTHYSIRSTLRMELMGCDLNSCSMPLGMESKAISDAQITASSYFT |
| | NMFATWSPSKARLHLQGRSNAWRPQVNNPKEWLQVDFQKTMKVTGVTTQGVKSLLTSMY |
| | VKEFLISSSQDGHQWTLFFQNGKVKVFQGNQDSFTPVVNSLDPPLLTRYLRIHPQSWVHQIA |
| | LRMEVLGCEAQDLY |
| FVIII BDD2 | ATRRYYLGAVELSWDYMQSDLGELPVDAGGAPSPSASTGTGPGTPGSGTASSSPGSSTPSGA |
| (A1-A28- | TGSPGPSGPGRFPPRVPKSFPFNTSVVYKKTLFVEFTVHLFNIAKPRPPWMGLLGPTIQAEVY |
| AG42-F29- | DTVVITLKNMASHPVSLHAVGVSYWKASEGAEYDDQTSQREKEGGPGTPGSGTASSSPGSS |
| E124- | TPSGATGSPGSSPSASTGTGPGASPGDDKVFPGGSHTYVWQVLKENGPMASDPLCLTYSYLS |
| AG42- | HVDLVKDLNSGLIGALLVCREGSLAKEKTQTLHKFILLFAVFDEGGSPSASTGTGPGASPGTS |
| D125-E124- | STGSPGTPGSGTASSSPGSSTPSGAGKSWHSETKNSLMQDRDAASARAWPKMHTVNGYVNS |
| AG42- | SLPGLIGCHRKSVYWHVIGMGTTPEVHSIFLEGHTFLVRNHRQASLEISPITFLTAQTLLMDL |
| D125-P333- | GQFLLFCHISSHQHDGMEAYVKVDSCPEEPGSASTGTGPGASPGTSSTGSPGTPGSGTASSSP |
| AG42- | GSSTPSGATGGQLRMKNNEEAEDYDDDLTDSEMDVVRFDDDNSPSFIQIRSVAKKHPKTWV |
| Q334- | HYIAAEEEDWDYAPLVLAPDDRSYKSQYLNNGPQRIGRKYKKVRFMAYTDETFKTREAIQH |
| Y2332) | ESGILGPLLYGEVGDTLLIIFKNQASRPYNIYPHGITDVRPLYSRRLPKGVKHLKDFPILPGEIF |
| | KYKWTVTVEDGPTKSDPRCLTRYYSSFVNMERDLASGLIGPLLICYKESVDQRGNQIMSDK |
| | RNVILFSVFDENRSWYLTENIQRFLPNPAGVQLEDPEFQASNIMHSINGYVFDSLQLSVCLHE |
| | VAYWYILSIGAQTDFLSVFFSGYTFKHKMVYEDTLTLFPFSGETVFMSMENPGLWILGCHNS |
| | DFRNRGMTALLKVSSCDKNTGDYYEDSYEDISAYLLSKNNAIEPRSFSQNPPVLKRHQREIT |
| | RTTLQSDQEEIDYDDTISVEMKKEDFDIYDEDENQSPRSFQKKTRHYFIAAVERLWDYGMSS |
| | SPHVLRNRAQSGSVPQFKKVVFQEFTDGSFTQPLYRGELNEHLGLLGPYIRAEVEDNIMVTF |
| | RNQASRPYSFYSSLISYEEDQRQGAEPRKNFVKPNETKTYFWKVQHHMAPTKDEFDCKAW |
| | AYFSDVDLEKDVHSGLIGPLLVCHTNTLNPAHGRQVTVQEFALFFTIFDETKSWYFTENMER |
| | NCRAPCNIQMEDPTFKENYRFHAINGYIMDTLPGLVMAQDQRIRWYLLSMGSNENIHSIHFS |
| | GHVFTVRKKEEYKMALYNLYPGVFETVEMLPSKAGIWRVECLIGEHLHAGMSTLFLVYSNK |
| | CQTPLGMASGHIRDFQITASGQYGQWAPKLARLHYSGSINAWSTKEPFSWIKVDLLAPMIIH |
| | GIKTQGARQKFSSLYISQFIIMYSLDGKKWQTYRGNSTGTLMVFFGNVDSSGIKHNIFNPPIIA |
| | RYIRLHPTHYSIRSTLRMELMGCDLNSCSMPLGMESKAISDAQITASSYFTNMFATWTPSKA |
| | RLHLQGRSNAWRPQVNNPKEWLQVDFQKTMKVTGVTTQGVKSLLTSMYVKEFLISSSQDG |
| | HQWTLFFQNGKVKVFQGNQDSFTPVVNSLDPPLLTRYLRIHPQSWVHQIALRMEVLGCEAQ |
| | DLY |
| FVIII | ATRRYYLGAVELSWDYMQSDLGELPVDARFPPRVPKSFPFNTSVVYKKTLFVEFTDHLFNIA |
| (A1-D345- | KPRPPWMGLLGPTIQAEVYDTVVITLKNMASHPVSLHAVGVSYWKASEGAEYDDQTSQRE |
| AE144- | KEDDKVFPGGSHTYVWQVLKENGPMASDPLCLTYSYLSHVDLVKDLNSGLIGALLVCREGS |
| Y346- | LAKEKTQTLHKFILLFAVFDEGKSWHSETKNSLMQDRDAASARAWPKMHTVNGYVNRSLP |
| D403- | GLIGCHRKSVYWHVIGMGTTPEVHSIFLEGHTFLVRNHRQASLEISPITFLTAQTLLMDLGQF |
| AE144- | LLFCHISSHQHDGMEAYVKVDSCPEEPQLRMKNNEEAEDGGSEPATSGSETPGTSESATPES |
| R405- | GPGSEPATSGSETPGSPAGSPTSTEEGTSTEPSEGSAPGSEPATSGSETPGSEPATSGSETPGSEP |
| R1797- | ATSGSETPGTSTEPSEGSAPGTSESATPESGPGSEPATSGSETPGTSTEPSEGSAPGYDDDLTDS |
| AE288- | EMDVVRFDDDNSPSFIQIRSVAKKHPKTWVHYIAAEEEDWDYAPLVLAPDDGGTSTPESGS |
| Q1798- | ASPGTSPSGESSTAPGTSPSGESSTAPGSTSSTAESPGPGSTSESPSGTAPGSTSSTAESPGPGTS |
| Y2322) | PSGESSTAPGTSTPESGSASPGSTSSTAESPGPGTSPSGESSTAPGTSPSGESSTAPGTSPSGESS |
| | TAPGRSYKSQYLNNGPQRIGRKYKKVRFMAYTDETFKTREAIQHESGILGPLLYGEVGDTLL |
| | IIFKNQASRPYNIYPHGITDVRPLYSRRLPKGVKHLKDFPILPGEIFKYKWTVTVEDGPTKSDP |
| | RCLTRYYSSFVNMERDLASGLIGPLLICYKESVDQRGNQIMSDKRNVILFSVFDENRSWYLT |
| | ENIQRFLPNPAGVQLEDPEFQASNIMHSINGYVFDSLQLSVCLHEVAYWYILSIGAQTDFLSV |
| | FFSGYTFKHKMVYEDTLTLFPFSGETVFMSMENPGLWILGCHNSDFRNRGMTALLKVSSCD |
| | KNTGDYYEDSYEDISAYLLSKNNAIEPRSFSQNSRHPSTRQKQFNATTIPENDIEKTDPWFAH |
| | RTPMPKIQNVSSSDLLMLLRQSPTPHGLSLSDLQEAKYETFSDDPSPGAIDSNNSLSEMTHFR |
| | PQLHHSGDMVFTPESGLQLRLNEKLGTTAATELKKLDFKVSSTSNNLISTIPSDNLAAGTDNT |
| | SSLGPPSMPVHYDSQLDTTLFGKKSSPLTESGGPLSLSEENNDSKLLESGLMNSQESSWGKN |
| | VSSTESGRLFKGKRAHGPALLTKDNALFKVSISLLKTNKTSNNSATNRKTHIDGPSLLIENSPS |
| | VWQNILESDTEFKKVTPLIHDRMLMDKNATALRLNHMSNKTTSSKNMEMVQQKKEGPIPP |
| | DAQNPDMSFFKMLFLPESARWIQRTHGKNSLNSGQGPSPKQLVSLGPEKSVEGQNFLSEKN |
| | KVVVGKGEFTKDVGLKEMVFPSSRNLFLTNLDNLHENNTHNQEKKIQEEIEKKETLIQENVV |
| | LPQIHTVTGTKNFMKNLFLLSTRQNVEGSYDGAYAPVLQDFRSLNDSTNRTKKHTAHFSKK |
| | GEEENLEGLGNQTKQIVEKYACTTRISPNTSQQNFVTQRSKRALKQFRLPLEETELEKRIIVD |
| | DTSTQWSKNMKHLTPSTLTQIDYNEKEKGAITQSPLSDCLTRSHSIPQANRSPLPIAKVSSFPSI |
| | RPIYLTRVLFQDNSSHLPAASYRKKDSGVQESSHFLQGAKKNNLSLAILTLEMTGDQREVGS |
| | LGTSATNSVTYKKVENTVLPKPDLPKTSGKVELLPKVHIYQKDLFPTETSNGSPGHLDLVEG |
| | SLLQGTEGAIKWNEANRPGKVPFLRVATESSAKTPSKLLDPLAWDNHYGTQIPKEEWKSQE |
| | KSPEKTAFKKKDTILSLNACESNHAIAAINEGQNKPEIEVTWAKQGRTERLCSQNPPVLKRH |
| | QREITRTTLQSDQEEIDYDDTISVEMKKEDFDIYDEDENQSPRSFQKKTRHYFIAAVERLWDY |
| | GMSSSPHVLRNRAQSGSVPQFKKVVFQEFTDGSFTQPLYRGELNEHLGLLGPYIRAEVEDNI |
| | MVTFRNQASRPYSFYSSLISYEEDQRGGTSESATPESGPGSEPATSGSETPGTSESATPESGPG |
| | SEPATSGSETPGTSESATPESGPGTSTEPSEGSAPGSPAGSPTSTEEGTSESATPESGPGSEPATS |
| | GSETPGTSESATPESGPGSPAGSPTSTEEGSPAGSPTSTEEGTSTEPSEGSAPGTSESATPESGPG |
| | TSESATPESGPGTSESATPESGPGSEPATSGSETPGSEPATSGSETPGSPAGSPTSTEEGTSTEPS |
| | EGSAPGTSTEPSEGSAPGSEPATSGSETPGTSESATPESGPGTSTEPSEGSAPGQGAEPRKNFV |
| | KPNETKTYFWKVQHHMAPTKDEFDCKAWAYFSDVDLEKDVHSGLIGPLLVCHTNTLNPAH |
| | GRQVTVQEFALFFTIFDETKSWYFTENMERNCRAPCNIQMEDPTFKENYRFHAINGYIMDTL |
| | PGLVMAQDQRIRWYLLSMGSNENIHSIHFSGHVFTVRKKEEYKMALYNLYPGVFETVEMLP |
| | SKAGIWRVECLIGEHLHAGMSTLFLVYSNKCQTPLGMASGHIRDFQITASGQYGQWAPKLA |
| | RLHYSGSINAWSTKEPFSWIKVDLLAPMIIHGIKTQGARQKFSSLYISQFIIMYSLDGKKWQT |
| | YRGNSTGTLMVFFGNVDSSGIKHNIFNPPIIARYIRLHPTHYSIRSTLRMELMGCDLNSCSMPL |
| | GMESKAISDAQITASSYFTNMFATWSPSKARLHLQGRSNAWRPQVNNPKEWLQVDFQKTM |
| | KVTGVTTQGVKSLLTSMYVKEFLISSSQDGHQWTLFFQNGKVKVFQGNQDSFTPVVNSLDP |
| | PLLTRYLRIHPQSWVHQIALRMEVLGCEAQDLY |
| FVIII (A1- | ATRRYYLGAVELSWDYMQSDLGELPVDARFPPRVPKSFPFNTSVVYKKTLFVEFTVHLFNIA |
| N745)- | KPRPPWMGLLGPTIQAEVYDTVVITLKNMASHPVSLHAVGVSYWKASEGAEYDDQTSQRE |
| AE864- | KEDDKVFPGGSHTYVWQVLKENGPMASDPLCLTYSYLSHVDLVKDLNSGLIGALLVCREGS |
| (P1640- | LAKEKTQTLHKFILLFAVFDEGKSWHSETKNSLMQDRDAASARAWPKMHTVNGYVNRSLP |
| Y2332) | GLIGCHRKSVYWHVIGMGTTPEVHSIFLEGHTFLVRNHRQASLEISPITFLTAQTLLMDLGQF |
| | LLFCHISSHQHDGMEAYVKVDSCPEEPQLRMKNNEEAEDYDDDLTDSEMDVVRFDDDNSP |
| | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSQNGGSPAGSPTSTEEGTSESATPESGPGTSTEPSEGSAPGSPAGSPTSTEEGTSTEPSEGSA |
| | PGTSTEPSEGSAPGTSESATPESGPGSEPATSGSETPGSEPATSGSETPGSPAGSPTSTEEGTSES |
| | ATPESGPGTSTEPSEGSAPGTSTEPSEGSAPGSPAGSPTSTEEGTSTEPSEGSAPGTSTEPSEGSA |
| | PGTSESATPESGPGTSTEPSEGSAPGTSESATPESGPGSEPATSGSETPGTSTEPSEGSAPGTSTE |
| | PSEGSAPGTSESATPESGPGTSESATPESGPGSPAGSPTSTEEGTSESATPESGPGSEPATSGSET |
| | PGTSESATPESGPGTSTEPSEGSAPGTSTEPSEGSAPGTSTEPSEGSAPGTSTEPSEGSAPGTSTE |
| | PSEGSAPGTSTEPSEGSAPGSPAGSPTSTEEGTSTEPSEGSAPGTSESATPESGPGSEPATSGSET |
| | PGTSESATPESGPGSEPATSGSETPGTSESATPESGPGTSTEPSEGSAPGTSESATPESGPGSPA |
| | GSPTSTEEGSPAGSPTSTEEGSPAGSPTSTEEGTSESATPESGPGTSTEPSEGSAPGTSESATPES |
| | GPGSEPATSGSETPGTSESATPESGPGSEPATSGSETPGTSESATPESGPGTSTEPSEGSAPGSP |
| | AGSPTSTEEGTSESATPESGPGSEPATSGSETPGTSESATPESGPGSPAGSPTSTEEGSPAGSPTS |
| | TEEGTSTEPSEGSAPGTSESATPESGPGTSESATPESGPGTSESATPESGPGSEPATSGSETPGSE |
| | PATSGSETPGSPAGSPTSTEEGTSTEPSEGSAPGTSTEPSEGSAPGSEPATSGSETPGTSESATPE |
| | SGPGTSTEPSEGSAPGPPVLKRHQREITRTTLQSDQEEIDYDDTISVEMKKEDFDIYDEDENQS |
| | PRSFQKKTRHYFIAAVERLWDYGMSSSPHVLRNRAQSGSVPQFKKVVFQEFTDGSFTQPLYR |
| | GELNEHLGLLGPYIRAEVEDNIMVTFRNQASRPYSFYSSLISYEEDQRQGAEPRKNFVKPNET |
| | KTYFWKVQHHMAPTKDEFDCKAWAYFSDVDLEKDVHSGLIGPLLVCHTNTLNPAHGRQVT |
| | VQEFALFFTIFDETKSWYFTENMERNCRAPCNIQMEDPTFKENYRFHAINGYIMDTLPGLVM |
| | AQDQRIRWYLLSMGSNENIHSIHFSGHVFTVRKKEEYKMALYNLYPGVFETVEMLPSKAGI |
| | WRVECLIGEHLHAGMSTLFLVYSNKCQTPLGMASGHIRDFQITASGQYGQWAPKLARLHYS |
| | GSINAWSTKEPFSWIKVDLLAPMIIHGIKTQGARQKFSSLYISQFIIMYSLDGKKWQTYRGNS |
| | TGTLMVFFGNVDSSGIKHNIFNPPIIARYIRLHPTHYSIRSTLRMELMGCDLNSCSMPLGMESK |
| | AISDAQITASSYFTNMFATWSPSKARLHLQGRSNAWRPQVNNPKEWLQVDFQKTMKVTGV |
| | TTQGVKSLLTSMYVKEFLISSSQDGHQWTLFFQNGKVKVFQGNQDSFTPVVNSLDPPLLTRY |
| | LRIHPQSWVHQIALRMEVLGCEAQDLY |
| FVIII BDD9 | ATRRYYLGAVELSWDYMQSDLGELPVDARFPPRVPKSFPFNTSVVYKKTLFVEFTVHLFNIA |
| (A1-N745)- | KPRPPWMGLLGPTIQAEVYDTVVITLKNMASHPVSLHAVGVSYWKASEGAEYDDQTSQRE |
| AE288- | KEDDKVFPGGSHTYVWQVLKENGPMASDPLCLTYSYLSHVDLVKDLNSGLIGALLVCREGS |
| (P1640- | LAKEKTQTLHKFILLFAVFDEGKSWHSETKNSLMQDRDAASARAWPKMHTVNGYVNRSLP |
| Y2332) | GLIGCHRKSVYWHVIGMGTTPEVHSIFLEGHTFLVRNHRQASLEISPITFLTAQTLLMDLGQF |
| | LLFCHISSHQHDGMEAYVKVDSCPEEPQLRMKNNEEAEDYDDDLTDSEMDVVRFDDDNSP |
| | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSQNGGTSESATPESGPGSEPATSGSETPGTSESATPESGPGSEPATSGSETPGTSESATPESG |
| | PGTSTEPSEGSAPGSPAGSPTSTEEGTSESATPESGPGSEPATSGSETPGTSESATPESGPGSPA |
| | GSPTSTEEGSPAGSPTSTEEGTSTEPSEGSAPGTSESATPESGPGTSESATPESGPGTSESATPES |
| | GPGSEPATSGSETPGSEPATSGSETPGSPAGSPTSTEEGTSTEPSEGSAPGTSTEPSEGSAPGSEP |
| | ATSGSETPGTSESATPESGPGTSTEPSEGSAPGPPVLKRHQREITRTTLQSDQEEIDYDDTISVE |
| | MKKEDFDIYDEDENQSPRSFQKKTRHYFIAAVERLWDYGMSSSPHVLRNRAQSGSVPQFKK |
| | VVFQEFTDGSFTQPLYRGELNEHLGLLGPYIRAEVEDNIMVTFRNQASRPYSFYSSLISYEED |
| | QRQGAEPRKNFVKPNETKTYFWKVQHHMAPTKDEFDCKAWAYFSDVDLEKDVHSGLIGPL |
| | LVCHTNTLNPAHGRQVTVQEFALFFTIFDETKSWYFTENMERNCRAPCNIQMEDPTFKENYR |
| | FHAINGYIMDTLPGLVMAQDQRIRWYLLSMGSNENIHSIHFSGHVFTVRKKEEYKMALYNL |
| | YPGVFETVEMLPSKAGIWRVECLIGEHLHAGMSTLFLVYSNKCQTPLGMASGHIRDFQITAS |
| | GQYGQWAPKLARLHYSGSINAWSTKEPFSWIKVDLLAPMIIHGIKTQGARQKFSSLYISQFII |
| | MYSLDGKKWQTYRGNSTGTLMVFFGNVDSSGIKHNIFNPPIIARYIRLHPTHYSIRSTLRMEL |
| | MGCDLNSCSMPLGMESKAISDAQITASSYFTNMFATWSPSKARLHLQGRSNAWRPQVNNPK |
| | EWLQVDFQKTMKVTGVTTQGVKSLLTSMYVKEFLISSSQDGHQWTLFFQNGKVKVFQGNQ |
| | DSFTPVVNSLDPPLLTRYLRIHPQSWVHQIALRMEVLGCEAQDLY |
| FVIII BDD9 | ATRRYYLGAVELSWDYMQSDLGELPVDARFPPRVPKSFPFNTSVVYKKTLFVEFTVHLFNIA |
| (A1-S743)- | KPRPPWMGLLGPTIQAEVYDTVVITLKNMASHPVSLHAVGVSYWKASEGAEYDDQTSQRE |
| AE288- | KEDDKVFPGGSHTYVWQVLKENGPMASDPLCLTYSYLSHVDLVKDLNSGLIGALLVCREGS |
| (Q1638- | LAKEKTQTLHKFILLFAVFDEGKSWHSETKNSLMQDRDAASARAWPKMHTVNGYVNRSLP |
| Y2332) | GLIGCHRKSVYWHVIGMGTTPEVHSIFLEGHTFLVRNHRQASLEISPITFLTAQTLLMDLGQF |
| | LLFCHISSHQHDGMEAYVKVDSCPEEPQLRMKNNEEAEDYDDDLTDSEMDVVRFDDDNSP |
| | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSGGTSESATPESGPGSEPATSGSETPGTSESATPESGPGSEPATSGSETPGTSESATPESGPGT |
| | STEPSEGSAPGSPAGSPTSTEEGTSESATPESGPGSEPATSGSETPGTSESATPESGPGSPAGSPT |
| | STEEGSPAGSPTSTEEGTSTEPSEGSAPGTSESATPESGPGTSESATPESGPGTSESATPESGPGS |
| | EPATSGSETPGSEPATSGSETPGSPAGSPTSTEEGTSTEPSEGSAPGTSTEPSEGSAPGSEPATSG |
| | SETPGTSESATPESGPGTSTEPSEGSAPGQNPPVLKRHQREITRTTLQSDQEEIDYDDTISVEM |
| | KKEDFDIYDEDENQSPRSFQKKTRHYFIAAVERLWDYGMSSSPHVLRNRAQSGSVPQFKKV |
| | VFQEFTDGSFTQPLYRGELNEHLGLLGPYIRAEVEDNIMVTFRNQASRPYSFYSSLISYEEDQ |
| | RQGAEPRKNFVKPNETKTYFWKVQHHMAPTKDEFDCKAWAYFSDVDLEKDVHSGLIGPLL |
| | VCHTNTLNPAHGRQVTVQEFALFFTIFDETKSWYFTENMERNCRAPCNIQMEDPTFKENYRF |
| | HAINGYIMDTLPGLVMAQDQRIRWYLLSMGSNENIHSIHFSGHVFTVRKKEEYKMALYNLY |
| | PGVFETVEMLPSKAGIWRVECLIGEHLHAGMSTLFLVYSNKCQTPLGMASGHIRDFQITASG |
| | QYGQWAPKLARLHYSGSINAWSTKEPFSWIKVDLLAPMIIHGIKTQGARQKFSSLYISQFIIM |
| | YSLDGKKWQTYRGNSTGTLMVFFGNVDSSGIKHNIFNPPIIARYIRLHPTHYSIRSTLRMELM |
| | GCDLNSCSMPLGMESKAISDAQITASSYFTNMFATWSPSKARLHLQGRSNAWRPQVNNPKE |
| | WLQVDFQKTMKVTGVTTQGVKSLLTSMYVKEFLISSSQDGHQWTLFFQNGKVKVFQGNQD |
| | SFTPVVNSLDPPLLTRYLRIHPQSWVHQIALRMEVLGCEAQDLY |
| FVIII BDD9 | ATRRYYLGAVELSWDYMQSDLGELPVDARFPPRVPKSFPFNTSVVYKKTLFVEFTVHLFNIA |
| (A1-N745)- | KPRPPWMGLLGPTIQAEVYDTVVITLKNMASHPVSLHAVGVSYWKASEGAEYDDQTSQRE |
| AG288_2- | KEDDKVFPGGSHTYVWQVLKENGPMASDPLCLTYSYLSHVDLVKDLNSGLIGALLVCREGS |
| (P1640- | LAKEKTQTLHKFILLFAVFDEGKSWHSETKNSLMQDRDAASARAWPKMHTVNGYVNRSLP |
| Y2332)- | GLIGCHRKSVYWHVIGMGTTPEVHSIFLEGHTFLVRNHRQASLEISPITFLTAQTLLMDLGQF |
| AG288_2 | LLFCHISSHQHDGMEAYVKVDSCPEEPQLRMKNNEEAEDYDDDLTDSEMDVVRFDDDNSP |
| | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSQNGPGASPGTSSTGSPGASPGTSSTGSPGTPGSGTASSSPGSSTPSGATGSPGTPGSGTASS |
| | SPGSSTPSGATGSPGTPGSGTASSSPGSSTPSGATGSPGSSTPSGATGSPGSSPSASTGTGPGSSP |
| | SASTGTGPGASPGTSSTGSPGTPGSGTASSSPGSSTPSGATGSPGSSPSASTGTGPGSSPSASTG |
| | TGPGASPGTSSTGSPGASPGTSSTGSPGSSTPSGATGSPGSSPSASTGTGPGASPGTSSTGSPGS |
| | SPSASTGTGPGTPGSGTASSSPGSSTPSGATGSGPPVLKRHQREITRTTLQSDQEEIDYDDTISV |
| | EMKKEDFDIYDEDENQSPRSFQKKTRHYFIAAVERLWDYGMSSSPHVLRNRAQSGSVPQFK |
| | KVVFQEFTDGSFTQPLYRGELNEHLGLLGPYIRAEVEDNIMVTFRNQASRPYSFYSSLISYEE |
| | DQRQGAEPRKNFVKPNETKTYFWKVQHHMAPTKDEFDCKAWAYFSDVDLEKDVHSGLIGP |
| | LLVCHTNTLNPAHGRQVTVQEFALFFTIFDETKSWYFTENMERNCRAPCNIQMEDPTFKENY |
| | RFHAINGYIMDTLPGLVMAQDQRIRWYLLSMGSNENIHSIHFSGHVFTVRKKEEYKMALYN |
| | LYPGVFETVEMLPSKAGIWRVECLIGEHLHAGMSTLFLVYSNKCQTPLGMASGHIRDFQITA |
| | SGQYGQWAPKLARLHYSGSINAWSTKEPFSWIKVDLLAPMIIHGIKTQGARQKFSSLYISQFII |
| | MYSLDGKKWQTYRGNSTGTLMVFFGNVDSSGIKHNIFNPPIIARYIRLHPTHYSIRSTLRMEL |
| | MGCDLNSCSMPLGMESKAISDAQITASSYFTNMFATWSPSKARLHLQGRSNAWRPQVNNPK |
| | EWLQVDFQKTMKVTGVTTQGVKSLLTSMYVKEFLISSSQDGHQWTLFFQNGKVKVFQGNQ |
| | DSFTPVVNSLDPPLLTRYLRIHPQSWVHQIALRMEVLGCEAQDLYGAGSPGAETAPGASPGT |
| | SSTGSPGASPGTSSTGSPGTPGSGTASSSPGSSTPSGATGSPGTPGSGTASSSPGSSTPSGATGS |
| | PGTPGSGTASSSPGSSTPSGATGSPGSSTPSGATGSPGSSPSASTGTGPGSSPSASTGTGPGASP |
| | GTSSTGSPGTPGSGTASSSPGSSTPSGATGSPGSSPSASTGTGPGSSPSASTGTGPGASPGTSST |
| | GSPGASPGTSSTGSPGSSTPSGATGSPGSSPSASTGTGPGASPGTSSTGSPGSSPSASTGTGPGT |
| | PGSGTASSSPGSSTPSGATGSGAETAEQKLISEEDLSPATG |
| FVIII BDD9 | ATRRYYLGAVELSWDYMQSDLGELPVDARFPPRVPKSFPFNTSVVYKKTLFVEFTVHLFNIA |
| (A1-S743)- | KPRPPWMGLLGPTIQAEVYDTVVITLKNMASHPVSLHAVGVSYWKASEGAEYDDQTSQRE |
| AG288_2- | KEDDKVFPGGSHTYVWQVLKENGPMASDPLCLTYSYLSHVDLVKDLNSGLIGALLVCREGS |
| (Q1638- | LAKEKTQTLHKFILLFAVFDEGKSWHSETKNSLMQDRDAASARAWPKMHTVNGYVNRSLP |
| Y2332)- | GLIGCHRKSVYWHVIGMGTTPEVHSIFLEGHTFLVRNHRQASLEISPITFLTAQTLLMDLGQF |
| AG288_2 | LLFCHISSHQHDGMEAYVKVDSCPEEPQLRMKNNEEAEDYDDDLTDSEMDVVRFDDDNSP |
| | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSGPGASPGTSSTGSPGASPGTSSTGSPGTPGSGTASSSPGSSTPSGATGSPGTPGSGTASSSP |
| | GSSTPSGATGSPGTPGSGTASSSPGSSTPSGATGSPGSSTPSGATGSPGSSPSASTGTGPGSSPS |
| | ASTGTGPGASPGTSSTGSPGTPGSGTASSSPGSSTPSGATGSPGSSPSASTGTGPGSSPSASTGT |
| | GPGASPGTSSTGSPGASPGTSSTGSPGSSTPSGATGSPGSSPSASTGTGPGASPGTSSTGSPGSS |
| | PSASTGTGPGTPGSGTASSSPGSSTPSGATGSGQNPPVLKRHQREITRTTLQSDQEEIDYDDTI |
| | SVEMKKEDFDIYDEDENQSPRSFQKKTRHYFIAAVERLWDYGMSSSPHVLRNRAQSGSVPQ |
| | FKKVVFQEFTDGSFTQPLYRGELNEHLGLLGPYIRAEVEDNIMVTFRNQASRPYSFYSSLISY |
| | EEDQRQGAEPRKNFVKPNETKTYFWKVQHHMAPTKDEFDCKAWAYFSDVDLEKDVHSGLI |
| | GPLLVCHTNTLNPAHGRQVTVQEFALFFTIFDETKSWYFTENMERNCRAPCNIQMEDPTFKE |
| | NYRFHAINGYIMDTLPGLVMAQDQRIRWYLLSMGSNENIHSIHFSGHVFTVRKKEEYKMAL |
| | YNLYPGVFETVEMLPSKAGIWRVECLIGEHLHAGMSTLFLVYSNKCQTPLGMASGHIRDFQI |
| | TASGQYGQWAPKLARLHYSGSINAWSTKEPFSWIKVDLLAPMIIHGIKTQGARQKFSSLYISQ |
| | FIIMYSLDGKKWQTYRGNSTGTLMVFFGNVDSSGIKHNIFNPPIIARYIRLHPTHYSIRSTLRM |
| | ELMGCDLNSCSMPLGMESKAISDAQITASSYFTNMFATWSPSKARLHLQGRSNAWRPQVNN |
| | PKEWLQVDFQKTMKVTGVTTQGVKSLLTSMYVKEFLISSSQDGHQWTLFFQNGKVKVFQG |
| | NQDSFTPVVNSLDPPLLTRYLRIHPQSWVHQIALRMEVLGCEAQDLYGAGSPGAETAPGASP |
| | GTSSTGSPGASPGTSSTGSPGTPGSGTASSSPGSSTPSGATGSPGTPGSGTASSSPGSSTPSGAT |
| | GSPGTPGSGTASSSPGSSTPSGATGSPGSSTPSGATGSPGSSPSASTGTGPGSSPSASTGTGPGA |
| | SPGTSSTGSPGTPGSGTASSSPGSSTPSGATGSPGSSPSASTGTGPGSSPSASTGTGPGASPGTS |
| | STGSPGASPGTSSTGSPGSSTPSGATGSPGSSPSASTGTGPGASPGTSSTGSPGSSPSASTGTGP |
| | GTPGSGTASSSPGSSTPSGATGSGAETAEQKLISEEDLSPATG |
| FVIII | ATRRYYLGAVELSWDYMQSDLGELPVDARFPPRVPKSFPFNTSVVYKKTLFVEFTDHLFNIA |
| BDD10 | KPRPPWMGLLGPTIQAEVYDTVVITLKNMASHPVSLHAVGVSYWKASEGAEYDDQTSQRE |
| (A1-N745)- | KEDDKVFPGGSHTYVWQVLKENGPMASDPLCLTYSYLSHVDLVKDLNSGLIGALLVCREGS |
| AE288- | LAKEKTQTLHKFILLFAVFDEGKSWHSETKNSLMQDRDAASARAWPKMHTVNGYVNRSLP |
| (P1640- | GLIGCHRKSVYWHVIGMGTTPEVHSIFLEGHTFLVRNHRQASLEISPITFLTAQTLLMDLGQF |
| Y2332)- | LLFCHISSHQHDGMEAYVKVDSCPEEPQLRMKNNEEAEDYDDDLTDSEMDVVRFDDDNSP |
| AE288 | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSQNG□GTSESATPESGPGSEPATSGSETPGTSESATPESGPGSEPATSGSETPGTSESATPES |
| | GPGTSTEPSEGSAPGSPAGSPTSTEEGTSESATPESGPGSEPATSGSETPGTSESATPESGPGSP |
| | AGSPTSTEEGSPAGSPTSTEEGTSTEPSEGSAPGTSESATPESGPGTSESATPESGPGTSESATPE |
| | SGPGSEPATSGSETPGSEPATSGSETPGSPAGSPTSTEEGTSTEPSEGSAPGTSTEPSEGSAPGSE |
| | PATSGSETPGTSESATPESGPGTSTEPSEGSAPGPPVLKRHQAEITRTTLQSDQEEIDYDDTISV |
| | EMKKEDFDIYDEDENQSPRSFQKKTRHYFIAAVERLWDYGMSSSPHVLRNRAQSGSVPQFK |
| | KVVFQEFTDGSFTQPLYRGELNEHLGLLGPYIRAEVEDNIMVTFRNQASRPYSFYSSLISYEE |
| | DQRQGAEPRKNFVKPNETKTYFWKVQHHMAPTKDEFDCKAWAYFSDVDLEKDVHSGLIGP |
| | LLVCHTNTLNPAHGRQVTVQEFALFFTIFDETKSWYFTENMERNCRAPCNIQMEDPTFKENY |
| | RFHAINGYIMDTLPGLVMAQDQRIRWYLLSMGSNENIHSIHFSGHVFTVRKKEEYKMALYN |
| | LYPGVFETVEMLPSKAGIWRVECLIGEHLHAGMSTLFLVYSNKCQTPLGMASGHIRDFQITA |
| | SGQYGQWAPKLARLHYSGSINAWSTKEPFSWIKVDLLAPMIIHGIKTQGARQKFSSLYISQFII |
| | MYSLDGKKWQTYRGNSTGTLMVFFGNVDSSGIKHNIFNPPIIARYIRLHPTHYSIRSTLRMEL |
| | MGCDLNSCSMPLGMESKAISDAQITASSYFTNMFATWSPSKARLHLQGRSNAWRPQVNNPK |
| | EWLQVDFQKTMKVTGVTTQGVKSLLTSMYVKEFLISSSQDGHQWTLFFQNGKVKVFQGNQ |
| | DSFTPVVNSLDPPLLTRYLRIHPQSWVHQIALRMEVLGCEAQDLYGGTSESATPESGPGSEPA |
| | TSGSETPGTSESATPESGPGSEPATSGSETPGTSESATPESGPGTSTEPSEGSAPGSPAGSPTSTE |
| | EGTSESATPESGPGSEPATSGSETPGTSESATPESGPGSPAGSPTSTEEGSPAGSPTSTEEGTSTE |
| | PSEGSAPGTSESATPESGPGTSESATPESGPGTSESATPESGPGSEPATSGSETPGSEPATSGSET |
| | PGSPAGSPTSTEEGTSTEPSEGSAPGTSTEPSEGSAPGSEPATSGSETPGTSESATPESGPGTSTE |
| | PSEGSAP |
| FVIII | ATRRYYLGAVELSWDYMQSDLGELPVDARFPPRVPKSFPFNTSVVYKKTLFVEFTDHLFNIA |
| BDD10 | KPRPPWMGLLGPTIQAEVYDTVVITLKNMASHPVSLHAVGVSYWKASEGAEYDDQTSQRE |
| (A1-S743)- | KEDDKVFPGGSHTYVWQVLKENGPMASDPLCLTYSYLSHVDLVKDLNSGLIGALLVCREGS |
| AE288- | LAKEKTQTLHKFILLFAVFDEGKSWHSETKNSLMQDRDAASARAWPKMHTVNGYVNRSLP |
| (Q1638- | GLIGCHRKSVYWHVIGMGTTPEVHSIFLEGHTFLVRNHRQASLEISPITFLTAQTLLMDLGQF |
| Y2332)- | LLFCHISSHQHDGMEAYVKVDSCPEEPQLRMKNNEEAEDYDDDLTDSEMDVVRFDDDNSP |
| AE288 | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSGGTSESATPESGPGSEPATSGSETPGTSESATPESGPGSEPATSGSETPGTSESATPESGPGT |
| | STEPSEGSAPGSPAGSPTSTEEGTSESATPESGPGSEPATSGSETPGTSESATPESGPGSPAGSPT |
| | STEEGSPAGSPTSTEEGTSTEPSEGSAPGTSESATPESGPGTSESATPESGPGTSESATPESGPGS |
| | EPATSGSETPGSEPATSGSETPGSPAGSPTSTEEGTSTEPSEGSAPGTSTEPSEGSAPGSEPATSG |
| | SETPGTSESATPESGPGTSTEPSEGSAPGQNPPVLKRHQAEITRTTLQSDQEEIDYDDTISVEM |
| | KKEDFDIYDEDENQSPRSFQKKTRHYFIAAVERLWDYGMSSSPHVLRNRAQSGSVPQFKKV |
| | VFQEFTDGSFTQPLYRGELNEHLGLLGPYIRAEVEDNIMVTFRNQASRPYSFYSSLISYEEDQ |
| | RQGAEPRKNFVKPNETKTYFWKVQHHMAPTKDEFDCKAWAYFSDVDLEKDVHSGLIGPLL |
| | VCHTNTLNPAHGRQVTVQEFALFFTIFDETKSWYFTENMERNCRAPCNIQMEDPTFKENYRF |
| | HAINGYIMDTLPGLVMAQDQRIRWYLLSMGSNENIHSIHFSGHVFTVRKKEEYKMALYNLY |
| | PGVFETVEMLPSKAGIWRVECLIGEHLHAGMSTLFLVYSNKCQTPLGMASGHIRDFQITASG |
| | QYGQWAPKLARLHYSGSINAWSTKEPFSWIKVDLLAPMIIHGIKTQGARQKFSSLYISQFIIM |
| | YSLDGKKWQTYRGNSTGTLMVFFGNVDSSGIKHNIFNPPIIARYIRLHPTHYSIRSTLRMELM |
| | GCDLNSCSMPLGMESKAISDAQITASSYFTNMFATWSPSKARLHLQGRSNAWRPQVNNPKE |
| | WLQVDFQKTMKVTGVTTQGVKSLLTSMYVKEFLISSSQDGHQWTLFFQNGKVKVFQGNQD |
| | SFTPVVNSLDPPLLTRYLRIHPQSWVHQIALRMEVLGCEAQDLYGGTSESATPESGPGSEPAT |
| | SGSETPGTSESATPESGPGSEPATSGSETPGTSESATPESGPGTSTEPSEGSAPGSPAGSPTSTEE |
| | GTSESATPESGPGSEPATSGSETPGTSESATPESGPGSPAGSPTSTEEGSPAGSPTSTEEGTSTEP |
| | SEGSAPGTSESATPESGPGTSESATPESGPGTSESATPESGPGSEPATSGSETPGSEPATSGSETP |
| | GSPAGSPTSTEEGTSTEPSEGSAPGTSTEPSEGSAPGSEPATSGSETPGTSESATPESGPGTSTEP |
| | SEGSAP |
| FVIII | ATRRYYLGAVELSWDYMQSDLGELPVDARFPPRVPKSFPFNTSVVYKKTLFVEFTDHLFNIA |
| BDD10 | KPRPPWMGLLGPTIQAEVYDTVVITLKNMASHPVSLHAVGVSYWKASEGAEYDDQTSQRE |
| (A1-N745)- | KEDDKVFPGGSHTYVWQVLKENGPMASDPLCLTYSYLSHVDLVKDLNSGLIGALLVCREGS |
| AG288_2- | LAKEKTQTLHKFILLFAVFDEGKSWHSETKNSLMQDRDAASARAWPKMHTVNGYVNRSLP |
| (P1640- | GLIGCHRKSVYWHVIGMGTTPEVHSIFLEGHTFLVRNHRQASLEISPITFLTAQTLLMDLGQF |
| Y2332)- | LLFCHISSHQHDGMEAYVKVDSCPEEPQLRMKNNEEAEDYDDDLTDSEMDVVRFDDDNSP |
| AG288_2 | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSQNGPGASPGTSSTGSPGASPGTSSTGSPGTPGSGTASSSPGSSTPSGATGSPGTPGSGTASS |
| | SPGSSTPSGATGSPGTPGSGTASSSPGSSTPSGATGSPGSSTPSGATGSPGSSPSASTGTGPGSSP |
| | SASTGTGPGASPGTSSTGSPGTPGSGTASSSPGSSTPSGATGSPGSSPSASTGTGPGSSPSASTG |
| | TGPGASPGTSSTGSPGASPGTSSTGSPGSSTPSGATGSPGSSPSASTGTGPGASPGTSSTGSPGS |
| | SPSASTGTGPGTPGSGTASSSPGSSTPSGATGSPPVLKRHQAEITRTTLQSDQEEIDYDDTISVE |
| | MKKEDFDIYDEDENQSPRSFQKKTRHYFIAAVERLWDYGMSSSPHVLRNRAQSGSVPQFKK |
| | VVFQEFTDGSFTQPLYRGELNEHLGLLGPYIRAEVEDNIMVTFRNQASRPYSFYSSLISYEED |
| | QRQGAEPRKNFVKPNETKTYFWKVQHHMAPTKDEFDCKAWAYFSDVDLEKDVHSGLIGPL |
| | LVCHTNTLNPAHGRQVTVQEFALFFTIFDETKSWYFTENMERNCRAPCNIQMEDPTFKENYR |
| | FHAINGYIMDTLPGLVMAQDQRIRWYLLSMGSNENIHSIHFSGHVFTVRKKEEYKMALYNL |
| | YPGVFETVEMLPSKAGIWRVECLIGEHLHAGMSTLFLVYSNKCQTPLGMASGHIRDFQITAS |
| | GQYGQWAPKLARLHYSGSINAWSTKEPFSWIKVDLLAPMIIHGIKTQGARQKFSSLYISQFII |
| | MYSLDGKKWQTYRGNSTGTLMVFFGNVDSSGIKHNIFNPPIIARYIRLHPTHYSIRSTLRMEL |
| | MGCDLNSCSMPLGMESKAISDAQITASSYFTNMFATWSPSKARLHLQGRSNAWRPQVNNPK |
| | EWLQVDFQKTMKVTGVTTQGVKSLLTSMYVKEFLISSSQDGHQWTLFFQNGKVKVFQGNQ |
| | DSFTPVVNSLDPPLLTRYLRIHPQSWVHQIALRMEVLGCEAQDLYGAGSPGAETAPGASPGT |
| | SSTGSPGASPGTSSTGSPGTPGSGTASSSPGSSTPSGATGSPGTPGSGTASSSPGSSTPSGATGS |
| | PGTPGSGTASSSPGSSTPSGATGSPGSSTPSGATGSPGSSPSASTGTGPGSSPSASTGTGPGASP |
| | GTSSTGSPGTPGSGTASSSPGSSTPSGATGSPGSSPSASTGTGPGSSPSASTGTGPGASPGTSST |
| | GSPGASPGTSSTGSPGSSTPSGATGSPGSSPSASTGTGPGASPGTSSTGSPGSSPSASTGTGPGT |
| | PGSGTASSSPGSSTPSGATGSGAETAEQKLISEEDLSPATG |
| FVIII | ATRRYYLGAVELSWDYMQSDLGELPVDARFPPRVPKSFPFNTSVVYKKTLFVEFTDHLFNIA |
| BDD10 | KPRPPWMGLLGPTIQAEVYDTVVITLKNMASHPVSLHAVGVSYWKASEGAEYDDQTSQRE |
| (A1-S743)- | KEDDKVFPGGSHTYVWQVLKENGPMASDPLCLTYSYLSHVDLVKDLNSGLIGALLVCREGS |
| AG288_2- | LAKEKTQTLHKFILLFAVFDEGKSWHSETKNSLMQDRDAASARAWPKMHTVNGYVNRSLP |
| (Q1638- | GLIGCHRKSVYWHVIGMGTTPEVHSIFLEGHTFLVRNHRQASLEISPITFLTAQTLLMDLGQF |
| Y2332)- | LLFCHISSHQHDGMEAYVKVDSCPEEPQLRMKNNEEAEDYDDDLTDSEMDVVRFDDDNSP |
| AG288_2 | SFIQIRSVAKKHPKTWVHYIAAEEEDWDYAPLVLAPDDRSYKSQYLNNGPQRIGRKYKKVR |
| | FMAYTDETFKTREAIQHESGILGPLLYGEVGDTLLIIFKNQASRPYNIYPHGITDVRPLYSRRL |
| | PKGVKHLKDFPILPGEIFKYKWTVTVEDGPTKSDPRCLTRYYSSFVNMERDLASGLIGPLLIC |
| | YKESVDQRGNQIMSDKRNVILFSVFDENRSWYLTENIQRFLPNPAGVQLEDPEFQASNIMHSI |
| | NGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPR |
| | SFSGPGASPGTSSTGSPGASPGTSSTGSPGTPGSGTASSSPGSSTPSGATGSPGTPGSGTASSSP |
| | GSSTPSGATGSPGTPGSGTASSSPGSSTPSGATGSPGSSTPSGATGSPGSSPSASTGTGPGSSPS |
| | ASTGTGPGASPGTSSTGSPGTPGSGTASSSPGSSTPSGATGSPGSSPSASTGTGPGSSPSASTGT |
| | GPGASPGTSSTGSPGASPGTSSTGSPGSSTPSGATGSPGSSPSASTGTGPGASPGTSSTGSPGSS |
| | PSASTGTGPGTPGSGTASSSPGSSTPSGATGSQNPPVLKRHQAEITRTTLQSDQEEIDYDDTIS |
| | VEMKKEDFDIYDEDENQSPRSFQKKTRHYFIAAVERLWDYGMSSSPHVLRNRAQSGSVPQF |
| | KKVVFQEFTDGSFTQPLYRGELNEHLGLLGPYIRAEVEDNIMVTFRNQASRPYSFYSSLISYE |
| | EDQRQGAEPRKNFVKPNETKTYFWKVQHHMAPTKDEFDCKAWAYFSDVDLEKDVHSGLI |
| | GPLLVCHTNTLNPAHGRQVTVQEFALFFTIFDETKSWYFTENMERNCRAPCNIQMEDPTFKE |
| | NYRFHAINGYIMDTLPGLVMAQDQRIRWYLLSMGSNENIHSIHFSGHVFTVRKKEEYKMAL |
| | YNLYPGVFETVEMLPSKAGIWRVECLIGEHLHAGMSTLFLVYSNKCQTPLGMASGHIRDFQI |
| | TASGQYGQWAPKLARLHYSGSINAWSTKEPFSWIKVDLLAPMIIHGIKTQGARQKFSSLYISQ |
| | FIIMYSLDGKKWQTYRGNSTGTLMVFFGNVDSSGIKHNIFNPPIIARYIRLHPTHYSIRSTLRM |
| | ELMGCDLNSCSMPLGMESKAISDAQITASSYFTNMFATWSPSKARLHLQGRSNAWRPQVNN |
| | PKEWLQVDFQKTMKVTGVTTQGVKSLLTSMYVKEFLISSSQDGHQWTLFFQNGKVKVFQG |
| | NQDSFTPVVNSLDPPLLTRYLRIHPQSWVHQIALRMEVLGCEAQDLYGAGSPGAETAPGASP |
| | GTSSTGSPGASPGTSSTGSPGTPGSGTASSSPGSSTPSGATGSPGTPGSGTASSSPGSSTPSGAT |
| | GSPGTPGSGTASSSPGSSTPSGATGSPGSSTPSGATGSPGSSPSASTGTGPGSSPSASTGTGPGA |
| | SPGTSSTGSPGTPGSGTASSSPGSSTPSGATGSPGSSPSASTGTGPGSSPSASTGTGPGASPGTS |
| | STGSPGASPGTSSTGSPGSSTPSGATGSPGSSPSASTGTGPGASPGTSSTGSPGSSPSASTGTGP |
| | GTPGSGTASSSPGSSTPSGATGSGAETAEQKLISEEDLSPATG |

*Sequence name reflects N- to C-terminus configuration of the FVIII segments (amino acid spanning numbers relative to mature sequence) and XTEN components

TABLE 30
Exemplary CFXTEN comprising FVIII, cleavage sequences and XTEN sequences

| CFXTEN | |
|---|---|
| Name* | Amino Acid Sequence |
| SP-AE288- | MQIELSTCFFLCLLRFCFSGTSESATPESGPGSEPATSGSETPGTSESATPESGPGSEPATSGSETP |
| CS-L- | GTSESATPESGPGTSTEPSEGSAPGSPAGSPTSTEEGTSESATPESGPGSEPATSGSETPGTSESA |
| (FVIII_1- | TPESGPGSPAGSPTSTEEGSPAGSPTSTEEGTSTEPSEGSAPGTSESATPESGPGTSESATPESGP |
| 745)- | GTSESATPESGPGSEPATSGSETPGSEPATSGSETPGSPAGSPTSTEEGTSTEPSEGSAPGTSTEPS |
| AE288- | EGSAPGSEPATSGSETPGTSESATPESGPGTSTEPSEGSAPQSPRSFQGPEGPSATRRYYLGAVE |
| (FVIII_1686- | LSWDYMQSDLGELPVDARFPPRVPKSFPFNTSVVYKKTLFVEFTVHLFNIAKPRPPWMGLLG |
| 2332)-L- | PTIQAEVYDTVVITLKNMASHPVSLHAVGVSYWKASEGAEYDDQTSQREKEDDKVFPGGSH |
| CS-AE288 | TYVWQVLKENGPMASDPLCLTYSYLSHVDLVKDLNSGLIGALLVCREGSLAKEKTQTLHKFI |
| | LLFAVFDEGKSWHSETKNSLMQDRDAASARAWPKMHTVNGYVNRSLPGLIGCHRKSVYWH |
| | VIGMGTTPEVHSIFLEGHTFLVRNHRQASLEISPITFLTAQTLLMDLGQFLLFCHISSHQHDGM |
| | EAYVKVDSCPEEPQLRMKNNEEAEDYDDDLTDSEMDVVRFDDDNSPSFIQIRSVAKKHPKT |
| | WVHYIAAEEEDWDYAPLVLAPDDRSYKSQYLNNGPQRIGRKYKKVRFMAYTDETFKTREAI |
| | QHESGILGPLLYGEVGDTLLIIFKNQASRPYNIYPHGITDVRPLYSRRLPKGVKHLKDFPILPGEI |
| | FKYKWTVTVEDGPTKSDPRCLTRYYSSFVNMERDLASGLIGPLLICYKESVDQRGNQIMSDK |
| | RNVILFSVFDENRSWYLTENIQRFLPNPAGVQLEDPEFQASNIMHSINGYVFDSLQLSVCLHEV |
| | AYWYILSIGAQTDFLSVFFSGYTFKHKMVYEDTLTLFPFSGETVFMSMENPGLWILGCHNSDF |
| | RNRGMTALLKVSSCDKNTGDYYEDSYEDISAYLLSKNNAIEPRSFSQNGTSESATPESGPGSEP |
| | ATSGSETPGTSESATPESGPGSEPATSGSETPGTSESATPESGPGTSTEPSEGSAPGSPAGSPTST |
| | EEGTSESATPESGPGSEPATSGSETPGTSESATPESGPGSPAGSPTSTEEGSPAGSPTSTEEGTST |
| | EPSEGSAPGTSESATPESGPGTSESATPESGPGTSESATPESGPGSEPATSGSETPGSEPATSGSE |
| | TPGSPAGSPTSTEEGTSTEPSEGSAPGTSTEPSEGSAPGSEPATSGSETPGTSESATPESGPGTST |
| | EPSEGSAPQSPRSFQKKTRHYFIAAVERLWDYGMSSSPHVLRNRAQSGSVPQFKKVVFQEFTD |
| | GSFTQPLYRGELNEHLGLLGPYIRAEVEDNIMVTFRNQASRPYSFYSSLISYEEDQRQGAEPRK |
| | NFVKPNETKTYFWKVQHHMAPTKDEFDCKAWAYFSDVDLEKDVHSGLIGPLLVCHTNTLNP |
| | AHGRQVTVQEFALFFTIFDETKSWYFTENMERNCRAPCNIQMEDPTFKENYRFHAINGYIMD |
| | TLPGLVMAQDQRIRWYLLSMGSNENIHSIHFSGHVFTVRKKEEYKMALYNLYPGVFETVEML |
| | PSKAGIWRVECLIGEHLHAGMSTLFLVYSNKCQTPLGMASGHIRDFQITASGQYGQWAPKLA |
| | RLHYSGSINAWSTKEPFSWIKVDLLAPMIIHGIKTQGARQKFSSLYISQFIIMYSLDGKKWQTY |
| | RGNSTGTLMVFFGNVDSSGIKHNIFNPPIIARYIRLHPTHYSIRSTLRMELMGCDLNSCSMPLG |
| | MESKAISDAQITASSYFTNMFATWSPSKARLHLQGRSNAWRPQVNNPKEWLQVDFQKTMKV |
| | TGVTTQGVKSLLTSMYVKEFLISSSQDGHQWTLFFQNGKVKVFQGNQDSFTPVVNSLDPPLL |
| | TRYLRIHPQSWVHQIALRMEVLGCEAQDLYGPEGPSQSPRSFQGTSESATPESGPGSEPATSGS |
| | ETPGTSESATPESGPGSEPATSGSETPGTSESATPESGPGTSTEPSEGSAPGSPAGSPTSTEEGTS |
| | ESATPESGPGSEPATSGSETPGTSESATPESGPGSPAGSPTSTEEGSPAGSPTSTEEGTSTEPSEGS |
| | APGTSESATPESGPGTSESATPESGPGTSESATPESGPGSEPATSGSETPGSEPATSGSETPGSPA |
| | GSPTSTEEGTSTEPSEGSAPGTSTEPSEGSAPGSEPATSGSETPGTSESATPESGPGTSTEPSEGS |
| | AP |
| SP-AE576- | MQIELSTCFFLCLLRFCFSGSPAGSPTSTEEGTSESATPESGPGTSTEPSEGSAPGSPAGSPTSTEE |
| CS-L- | GTSTEPSEGSAPGTSTEPSEGSAPGTSESATPESGPGSEPATSGSETPGSEPATSGSETPGSPAGS |
| (FVIII_1- | PTSTEEGTSESATPESGPGTSTEPSEGSAPGTSTEPSEGSAPGSPAGSPTSTEEGTSTEPSEGSAP |
| 745)- | GTSTEPSEGSAPGTSESATPESGPGTSTEPSEGSAPGTSESATPESGPGSEPATSGSETPGTSTEPS |
| AE576- | EGSAPGTSTEPSEGSAPGTSESATPESGPGTSESATPESGPGSPAGSPTSTEEGTSESATPESGPG |
| (FVIII_1686- | SEPATSGSETPGTSESATPESGPGTSTEPSEGSAPGTSTEPSEGSAPGTSTEPSEGSAPGTSTEPSE |
| 2332)-L- | GSAPGTSTEPSEGSAPGTSTEPSEGSAPGSPAGSPTSTEEGTSTEPSEGSAPGTSESATPESGPGS |
| CS-AE288 | EPATSGSETPGTSESATPESGPGSEPATSGSETPGTSESATPESGPGTSTEPSEGSAPGTSESATP |
| | ESGPGSPAGSPTSTEEGSPAGSPTSTEEGSPAGSPTSTEEGTSESATPESGPGTSTEPSEGSAPQS |
| | PRSFQGPSGPATRRYYLGAVELSWDYMQSDLGELPVDARFPPRVPKSFPFNTSVVYKKTLFV |
| | EFTVHLFNIAKPRPPWMGLLGPTIQAEVYDTVVITLKNMASHPVSLHAVGVSYWKASEGAEY |
| | DDQTSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHTVNG |
| | YVNRSLPGLIGCHRKSVYWHVIGMGTTPEVHSIFLEGHTFLVRNHRQASLEISPITFLTAQTLL |
| | MDLGQFLLFCHISSHQHDGMEAYVKVDSCPEEPQLRMKNNEEAEDYDDDLTDSEMDVVRFD |
| | DDNSPSFIQIRSVAKKHPKTWVHYIAAEEEDWDYAPLVLAPDDRSYKSQYLNNGPQRIGRKY |
| | KKVRFMAYTDETFKTREAIQHESGILGPLLYGEVGDTLLIIFKNQASRPYNIYPHGITDVRPLY |
| | SRRLPKGVKHLKDFPILPGEIFKYKWTVTVEDGPTKSDPRCLTRYYSSFVNMERDLASGLIGP |
| | LLICYKESVDQRGNQIMSDKRNVILFSVFDENRSWYLTENIQRFLPNPAGVQLEDPEFQASNI |
| | MHSINGYVFDSLQLSVCLHEVAYWYILSIGAQTDFLSVFFSGYTFKHKMVYEDTLTLFPFSGE |
| | TVFMSMENPGLWILGCHNSDFRNRGMTALLKVSSCDKNTGDYYEDSYEDISAYLLSKNNAIE |
| | PRSFSQNGSPAGSPTSTEEGTSESATPESGPGTSTEPSEGSAPGSPAGSPTSTEEGTSTEPSEGSA |
| | PGTSTEPSEGSAPGTSESATPESGPGSEPATSGSETPGSEPATSGSETPGSPAGSPTSTEEGTSES |
| | ATPESGPGTSTEPSEGSAPGTSTEPSEGSAPGSPAGSPTSTEEGTSTEPSEGSAPGTSTEPSEGSA |
| | PGTSESATPESGPGTSTEPSEGSAPGTSESATPESGPGSEPATSGSETPGTSTEPSEGSAPGTSTEP |
| | SEGSAPGTSESATPESGPGTSESATPESGPGSPAGSPTSTEEGTSESATPESGPGSEPATSGSETP |
| | GTSESATPESGPGTSTEPSEGSAPGTSTEPSEGSAPGTSTEPSEGSAPGTSTEPSEGSAPGTSTEPS |
| | EGSAPGTSTEPSEGSAPGSPAGSPTSTEEGTSTEPSEGSAPGTSESATPESGPGSEPATSGSETPG |
| | TSESATPESGPGSEPATSGSETPGTSESATPESGPGTSTEPSEGSAPGTSESATPESGPGSPAGSP |
| | TSTEEGSPAGSPTSTEEGSPAGSPTSTEEGTSESATPESGPGTSTEPSEGSAPQSPRSFQKKTRHY |
| | FIAAVERLWDYGMSSSPHVLRNRAQSGSVPQFKKVVFQEFTDGSFTQPLYRGELNEHLGLLG |
| | PYIRAEVEDNIMVTFRNQASRPYSFYSSLISYEEDQRQGAEPRKNFVKPNETKTYFWKVQHH |
| | MAPTKDEFDCKAWAYFSDVDLEKDVHSGLIGPLLVCHTNTLNPAHGRQVTVQEFALFFTIFD |
| | ETKSWYFTENMERNCRAPCNIQMEDPTFKENYRFHAINGYIMDTLPGLVMAQDQRIRWYLLS |
| | MGSNENIHSIHFSGHVFTVRKKEEYKMALYNLYPGVFETVEMLPSKAGIWRVECLIGEHLHA |
| | GMSTLFLVYSNKCQTPLGMASGHIRDFQITASGQYGQWAPKLARLHYSGSINAWSTKEPFSW |
| | IKVDLLAPMIIHGIKTQGARQKFSSLYISQFIIMYSLDGKKWQTYRGNSTGTLMVFFGNVDSSG |
| | IKHNIFNPPIIARYIRLHPTHYSIRSTLRMELMGCDLNSCSMPLGMESKAISDAQITASSYFTNM |
| | FATWSPSKARLHLQGRSNAWRPQVNNPKEWLQVDFQKTMKVTGVTTQGVKSLLTSMYVKE |
| | FLISSSQDGHQWTLFFQNGKVKVFQGNQDSFTPVVNSLDPPLLTRYLRIHPQSWVHQIALRME |
| | VLGCEAQDLYGPEGPSQSPRSFQGTSESATPESGPGSEPATSGSETPGTSESATPESGPGSEPAT |
| | SGSETPGTSESATPESGPGTSTEPSEGSAPGSPAGSPTSTEEGTSESATPESGPGSEPATSGSETP |
| | GTSESATPESGPGSPAGSPTSTEEGSPAGSPTSTEEGTSTEPSEGSAPGTSESATPESGPGTSESA |
| | TPESGPGTSESATPESGPGSEPATSGSETPGSEPATSGSETPGSPAGSPTSTEEGTSTEPSEGSAP |
| | GTSTEPSEGSAPGSEPATSGSETPGTSESATPESGPGTSTEPSEGSAP |
| SP- | MQIELSTCFFLCLLRFCFSATRRYYLGAVELSWDYMQSDLGELPVDARFPPRVPKSFPFNTSV |
| (FVIII_1- | VYKKTLFVEFTVHLFNIAKPRPPWMGLLGPTIQAEVYDTVVITLKNMASHPVSLHAVGVSYW |
| 745)- | KASEGAEYDDQTSQREKEDDKVFPGGSHTYVWQVLKENGPMASDPLCLTYSYLSHVDLVK |
| AE576- | DLNSGLIGALLVCREGSLAKEKTQTLHKFILLFAVFDEGKSWHSETKNSLMQDRDAASARAW |
| (FVIII_1686- | PKMHTVNGYVNRSLPGLIGCHRKSVYWHVIGMGTTPEVHSIFLEGHTFLVRNHRQASLEISPI |
| 2332)-L- | TFLTAQTLLMDLGQFLLFCHISSHQHDGMEAYVKVDSCPEEPQLRMKNNEEAEDYDDDLTD |
| CS-AE576 | SEMDVVRFDDDNSPSFIQIRSVAKKHPKTWVHYIAAEEEDWDYAPLVLAPDDRSYKSQYLNN |
| | GPQRIGRKYKKVRFMAYTDETFKTREAIQHESGILGPLLYGEVGDTLLIIFKNQASRPYNIYPH |
| | GITDVRPLYSRRLPKGVKHLKDFPILPGEIFKYKWTVTVEDGPTKSDPRCLTRYYSSFVNMER |
| | DLASGLIGPLLICYKESVDQRGNQIMSDKRNVILFSVFDENRSWYLTENIQRFLPNPAGVQLED |
| | PEFQASNIMHSINGYVFDSLQLSVCLHEVAYWYILSIGAQTDFLSVFFSGYTFKHKMVYEDTL |
| | TLFPFSGETVFMSMENPGLWILGCHNSDFRNRGMTALLKVSSCDKNTGDYYEDSYEDISAYL |
| | LSKNNAIEPRSFSQNGSPAGSPTSTEEGTSESATPESGPGTSTEPSEGSAPGSPAGSPTSTEEGTS |
| | TEPSEGSAPGTSTEPSEGSAPGTSESATPESGPGSEPATSGSETPGSEPATSGSETPGSPAGSPTS |
| | TEEGTSESATPESGPGTSTEPSEGSAPGTSTEPSEGSAPGSPAGSPTSTEEGTSTEPSEGSAPGTS |
| | TEPSEGSAPGTSESATPESGPGTSTEPSEGSAPGTSESATPESGPGSEPATSGSETPGTSTEPSEGS |
| | APGTSTEPSEGSAPGTSESATPESGPGTSESATPESGPGSPAGSPTSTEEGTSESATPESGPGSEP |
| | ATSGSETPGTSESATPESGPGTSTEPSEGSAPGTSTEPSEGSAPGTSTEPSEGSAPGTSTEPSEGS |
| | APGTSTEPSEGSAPGTSTEPSEGSAPGSPAGSPTSTEEGTSTEPSEGSAPGTSESATPESGPGSEP |
| | ATSGSETPGTSESATPESGPGSEPATSGSETPGTSESATPESGPGTSTEPSEGSAPGTSESATPES |
| | GPGSPAGSPTSTEEGSPAGSPTSTEEGSPAGSPTSTEEGTSESATPESGPGTSTEPSEGSAPQSPR |
| | SFQKKTRHYFIAAVERLWDYGMSSSPHVLRNRAQSGSVPQFKKVVFQEFTDGSFTQPLYRGE |
| | LNEHLGLLGPYIRAEVEDNIMVTFRNQASRPYSFYSSLISYEEDQRQGAEPRKNFVKPNETKT |
| | YFWKVQHHMAPTKDEFDCKAWAYFSDVDLEKDVHSGLIGPLLVCHTNTLNPAHGRQVTVQ |
| | EFALFFTIFDETKSWYFTENMERNCRAPCNIQMEDPTFKENYRFHAINGYIMDTLPGLVMAQD |
| | QRIRWYLLSMGSNENIHSIHFSGHVFTVRKKEEYKMALYNLYPGVFETVEMLPSKAGIWRVE |
| | CLIGEHLHAGMSTLFLVYSNKCQTPLGMASGHIRDFQITASGQYGQWAPKLARLHYSGSINA |
| | WSTKEPFSWIKVDLLAPMIIHGIKTQGARQKFSSLYISQFIIMYSLDGKKWQTYRGNSTGTLM |
| | VFFGNVDSSGIKHNIFNPPIIARYIRLHPTHYSIRSTLRMELMGCDLNSCSMPLGMESKAISDAQ |
| | ITASSYFTNMFATWSPSKARLHLQGRSNAWRPQVNNPKEWLQVDFQKTMKVTGVTTQGVK |
| | SLLTSMYVKEFLISSSQDGHQWTLFFQNGKVKVFQGNQDSFTPVVNSLDPPLLTRYLRIHPQS |
| | WVHQIALRMEVLGCEAQDLYGPEGPSQSPRSFQGSPAGSPTSTEEGTSESATPESGPGTSTEPS |
| | EGSAPGSPAGSPTSTEEGTSTEPSEGSAPGTSTEPSEGSAPGTSESATPESGPGSEPATSGSETPG |
| | SEPATSGSETPGSPAGSPTSTEEGTSESATPESGPGTSTEPSEGSAPGTSTEPSEGSAPGSPAGSP |
| | TSTEEGTSTEPSEGSAPGTSTEPSEGSAPGTSESATPESGPGTSTEPSEGSAPGTSESATPESGPG |
| | SEPATSGSETPGTSTEPSEGSAPGTSTEPSEGSAPGTSESATPESGPGTSESATPESGPGSPAGSP |
| | TSTEEGTSESATPESGPGSEPATSGSETPGTSESATPESGPGTSTEPSEGSAPGTSTEPSEGSAPG |
| | TSTEPSEGSAPGTSTEPSEGSAPGTSTEPSEGSAPGTSTEPSEGSAPGSPAGSPTSTEEGTSTEPSE |
| | GSAPGTSESATPESGPGSEPATSGSETPGTSESATPESGPGSEPATSGSETPGTSESATPESGPGT |
| | STEPSEGSAPGTSESATPESGPGSPAGSPTSTEEGSPAGSPTSTEEGSPAGSPTSTEEGTSESATPE |
| | SGPGTSTEPSEGSAP |
| SP-AE576- | MQIELSTCFFLCLLRFCFSGSPAGSPTSTEEGTSESATPESGPGTSTEPSEGSAPGSPAGSPTSTEE |
| CS-L- | GTSTEPSEGSAPGTSTEPSEGSAPGTSESATPESGPGSEPATSGSETPGSEPATSGSETPGSPAGS |
| (FVIII_1- | PTSTEEGTSESATPESGPGTSTEPSEGSAPGTSTEPSEGSAPGSPAGSPTSTEEGTSTEPSEGSAP |
| 745)- | GTSTEPSEGSAPGTSESATPESGPGTSTEPSEGSAPGTSESATPESGPGSEPATSGSETPGTSTEPS |
| AE576- | EGSAPGTSTEPSEGSAPGTSESATPESGPGTSESATPESGPGSPAGSPTSTEEGTSESATPESGPG |
| (FVIII_1686- | SEPATSGSETPGTSESATPESGPGTSTEPSEGSAPGTSTEPSEGSAPGTSTEPSEGSAPGTSTEPSE |
| 2332) | GSAPGTSTEPSEGSAPGTSTEPSEGSAPGSPAGSPTSTEEGTSTEPSEGSAPGTSESATPESGPGS |
| | EPATSGSETPGTSESATPESGPGSEPATSGSETPGTSESATPESGPGTSTEPSEGSAPGTSESATP |
| | ESGPGSPAGSPTSTEEGSPAGSPTSTEEGSPAGSPTSTEEGTSESATPESGPGTSTEPSEGSAPQS |
| | PRSFQGPEGPSATRRYYLGAVELSWDYMQSDLGELPVDARFPPRVPKSFPFNTSVVYKKTLF |
| | VEFTVHLFNIAKPRPPWMGLLGPTIQAEVYDTVVITLKNMASHPVSLHAVGVSYWKASEGAE |
| | YDDQTSQREKEDDKVFPGGSHTYVWQVLKENGPMASDPLCLTYSYLSHVDLVKDLNSGLIG |
| | ALLVCREGSLAKEKTQTLHKFILLFAVFDEGKSWHSETKNSLMQDRDAASARAWPKMHTVN |
| | GYVNRSLPGLIGCHRKSVYWHVIGMGTTPEVHSIFLEGHTFLVRNHRQASLEISPITFLTAQTL |
| | LMDLGQFLLFCHISSHQHDGMEAYVKVDSCPEEPQLRMKNNEEAEDYDDDLTDSEMDVVRF |
| | DDDNSPSFIQIRSVAKKHPKTWVHYIAAEEEDWDYAPLVLAPDDRSYKSQYLNNGPQRIGRK |
| | YKKVRFMAYTDETFKTREAIQHESGILGPLLYGEVGDTLLIIFKNQASRPYNIYPHGITDVRPL |
| | YSRRLPKGVKHLKDFPILPGEIFKYKWTVTVEDGPTKSDPRCLTRYYSSFVNMERDLASGLIG |
| | PLLICYKESVDQRGNQIMSDKRNVILFSVFDENRSWYLTENIQRFLPNPAGVQLEDPEFQASNI |
| | MHSINGYVFDSLQLSVCLHEVAYWYILSIGAQTDFLSVFFSGYTFKHKMVYEDTLTLFPFSGE |
| | TVFMSMENPGLWILGCHNSDFRNRGMTALLKVSSCDKNTGDYYEDSYEDISAYLLSKNNAIE |
| | PRSFSQNGSPAGSPTSTEEGTSESATPESGPGTSTEPSEGSAPGSPAGSPTSTEEGTSTEPSEGSA |
| | PGTSTEPSEGSAPGTSESATPESGPGSEPATSGSETPGSEPATSGSETPGSPAGSPTSTEEGTSES |
| | ATPESGPGTSTEPSEGSAPGTSTEPSEGSAPGSPAGSPTSTEEGTSTEPSEGSAPGTSTEPSEGSA |
| | PGTSESATPESGPGTSTEPSEGSAPGTSESATPESGPGSEPATSGSETPGTSTEPSEGSAPGTSTEP |
| | SEGSAPGTSESATPESGPGTSESATPESGPGSPAGSPTSTEEGTSESATPESGPGSEPATSGSETP |
| | GTSESATPESGPGTSTEPSEGSAPGTSTEPSEGSAPGTSTEPSEGSAPGTSTEPSEGSAPGTSTEPS |
| | EGSAPGTSTEPSEGSAPGSPAGSPTSTEEGTSTEPSEGSAPGTSESATPESGPGSEPATSGSETPG |
| | TSESATPESGPGSEPATSGSETPGTSESATPESGPGTSTEPSEGSAPGTSESATPESGPGSPAGSP |
| | TSTEEGSPAGSPTSTEEGSPAGSPTSTEEGTSESATPESGPGTSTEPSEGSAPQSPRSFQKKTRHY |
| | FIAAVERLWDYGMSSSPHVLRNRAQSGSVPQFKKVVFQEFTDGSFTQPLYRGELNEHLGLLG |
| | PYIRAEVEDNIMVTFRNQASRPYSFYSSLISYEEDQRQGAEPRKNFVKPNETKTYFWKVQHH |
| | MAPTKDEFDCKAWAYFSDVDLEKDVHSGLIGPLLVCHTNTLNPAHGRQVTVQEFALFFTIFD |
| | ETKSWYFTENMERNCRAPCNIQMEDPTFKENYRFHAINGYIMDTLPGLVMAQDQRIRWYLLS |
| | MGSNENIHSIHFSGHVFTVRKKEEYKMALYNLYPGVFETVEMLPSKAGIWRVECLIGEHLHA |
| | GMSTLFLVYSNKCQTPLGMASGHIRDFQITASGQYGQWAPKLARLHYSGSINAWSTKEPFSW |
| | IKVDLLAPMIIHGIKTQGARQKFSSLYISQFIIMYSLDGKKWQTYRGNSTGTLMVFFGNVDSSG |
| | IKHNIFNPPIIARYIRLHPTHYSIRSTLRMELMGCDLNSCSMPLGMESKAISDAQITASSYFTNM |
| | FATWSPSKARLHLQGRSNAWRPQVNNPKEWLQVDFQKTMKVTGVTTQGVKSLLTSMYVKE |
| | FLISSSQDGHQWTLFFQNGKVKVFQGNQDSFTPVVNSLDPPLLTRYLRIHPQSWVHQIALRME |
| | VLGCEAQDLY |
| SP-AE576- | MQIELSTCFFLCLLRFCFSGSPAGSPTSTEEGTSESATPESGPGTSTEPSEGSAPGSPAGSPTSTEE |
| CS-L- | GTSTEPSEGSAPGTSTEPSEGSAPGTSESATPESGPGSEPATSGSETPGSEPATSGSETPGSPAGS |
| (FVIII_1- | PTSTEEGTSESATPESGPGTSTEPSEGSAPGTSTEPSEGSAPGSPAGSPTSTEEGTSTEPSEGSAP |
| 743)- | GTSTEPSEGSAPGTSESATPESGPGTSTEPSEGSAPGTSESATPESGPGSEPATSGSETPGTSTEPS |
| AE288- | EGSAPGTSTEPSEGSAPGTSESATPESGPGTSESATPESGPGSPAGSPTSTEEGTSESATPESGPG |
| (FVIII_1686- | SEPATSGSETPGTSESATPESGPGTSTEPSEGSAPGTSTEPSEGSAPGTSTEPSEGSAPGTSTEPSE |
| 2332)-L- | GSAPGTSTEPSEGSAPGTSTEPSEGSAPGSPAGSPTSTEEGTSTEPSEGSAPGTSESATPESGPGS |
| CS-AE576 | EPATSGSETPGTSESATPESGPGSEPATSGSETPGTSESATPESGPGTSTEPSEGSAPGTSESATP |
| | ESGPGSPAGSPTSTEEGSPAGSPTSTEEGSPAGSPTSTEEGTSESATPESGPGTSTEPSEGSAPIEP |
| | RSPSGSPGATRRYYLGAVELSWDYMQSDLGELPVDARFPPRVPKSFPFNTSVVYKKTLFVEFT |
| | VHLFNIAKPRPPWMGLLGPTIQAEVYDTVVITLKNMASHPVSLHAVGVSYWKASEGAEYDD |
| | QTSQREKEDDKVFPGGSHTYVWQVLKENGPMASDPLCLTYSYLSHVDLVKDLNSGLIGALL |
| | VCREGSLAKEKTQTLHKFILLFAVFDEGKSWHSETKNSLMQDRDAASARAWPKMHTVNGY |
| | VNRSLPGLIGCHRKSVYWHVIGMGTTPEVHSIFLEGHTFLVRNHRQASLEISPITFLTAQTLLM |
| | DLGQFLLFCHISSHQHDGMEAYVKVDSCPEEPQLRMKNNEEAEDYDDDLTDSEMDVVRFDD |
| | DNSPSFIQIRSVAKKHPKTWVHYIAAEEEDWDYAPLVLAPDDRSYKSQYLNNGPQRIGRKYK |
| | KVRFMAYTDETFKTREAIQHESGILGPLLYGEVGDTLLIIFKNQASRPYNIYPHGITDVRPLYSR |
| | RLPKGVKHLKDFPILPGEIFKYKWTVTVEDGPTKSDPRCLTRYYSSFVNMERDLASGLIGPLLI |
| | CYKESVDQRGNQIMSDKRNVILFSVFDENRSWYLTENIQRFLPNPAGVQLEDPEFQASNIMHS |
| | INGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPRS |
| | FSGTSESATPESGPGSEPATSGSETPGTSESATPESGPGSEPATSGSETPGTSESATPESGPGTSTE |
| | PSEGSAPGSPAGSPTSTEEGTSESATPESGPGSEPATSGSETPGTSESATPESGPGSPAGSPTSTE |
| | EGSPAGSPTSTEEGTSTEPSEGSAPGTSESATPESGPGTSESATPESGPGTSESATPESGPGSEPA |
| | TSGSETPGSEPATSGSETPGSPAGSPTSTEEGTSTEPSEGSAPGTSTEPSEGSAPGSEPATSGSETP |
| | GTSESATPESGPGTSTEPSEGSAPQSPRSFQKKTRHYFIAAVERLWDYGMSSSPHVLRNRAQS |
| | GSVPQFKKVVFQEFTDGSFTQPLYRGELNEHLGLLGPYIRAEVEDNIMVTFRNQASRPYSFYS |
| | SLISYEEDQRQGAEPRKNFVKPNETKTYFWKVQHHMAPTKDEFDCKAWAYFSDVDLEKDV |
| | HSGLIGPLLVCHTNTLNPAHGRQVTVQEFALFFTIFDETKSWYFTENMERNCRAPCNIQMEDP |
| | TFKENYRFHAINGYIMDTLPGLVMAQDQRIRWYLLSMGSNENIHSIHFSGHVFTVRKKEEYK |
| | MALYNLYPGVFETVEMLPSKAGIWRVECLIGEHLHAGMSTLFLVYSNKCQTPLGMASGHIRD |
| | FQITASGQYGQWAPKLARLHYSGSINAWSTKEPFSWIKVDLLAPMIIHGIKTQGARQKFSSLYI |
| | SQFIIMYSLDGKKWQTYRGNSTGTLMVFFGNVDSSGIKHNIFNPPIIARYIRLHPTHYSIRSTLR |
| | MELMGCDLNSCSMPLGMESKAISDAQITASSYFTNMFATWSPSKARLHLQGRSNAWRPQVN |
| | NPKEWLQVDFQKTMKVTGVTTQGVKSLLTSMYVKEFLISSSQDGHQWTLFFQNGKVKVFQG |
| | NQDSFTPVVNSLDPPLLTRYLRIHPQSWVHQIALRMEVLGCEAQDLYGSPGIEPRSPSGSPAGS |
| | PTSTEEGTSESATPESGPGTSTEPSEGSAPGSPAGSPTSTEEGTSTEPSEGSAPGTSTEPSEGSAP |
| | GTSESATPESGPGSEPATSGSETPGSEPATSGSETPGSPAGSPTSTEEGTSESATPESGPGTSTEPS |
| | EGSAPGTSTEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSAPGTSESATP |
| | ESGPGTSESATPESGPGSPAGSPTSTEEGTSESATPESGPGSEPATSGSETPGTSESATPESGPGT |
| | STEPSEGSAPGTSTEPSEGSAPGTSTEPSEGSAPGTSTEPSEGSAPGTSTEPSEGSAPGTSTEPSEG |
| | SAPGSPAGSPTSTEEGTSTEPSEGSAPGTSESATPESGPGSEPATSGSETPGTSESATPESGPGSE |
| | PATSGSETPGTSESATPESGPGTSTEPSEGSAPGTSESATPESGPGSPAGSPTSTEEGSPAGSPTS |
| | TEEGSPAGSPTSTEEGTSESATPESGPGTSTEPSEGSAP |
| SP-AG288- | MQIELSTCFFLCLLRFCFSPGASPGTSSTGSPGASPGTSSTGSPGTPGSGTASSSPGSSTPSGATG |
| CS-L- | SPGTPGSGTASSSPGSSTPSGATGSPGTPGSGTASSSPGSSTPSGATGSPGSSTPSGATGSPGSSP |
| (FVIII_1- | SASTGTGPGSSPSASTGTGPGASPGTSSTGSPGTPGSGTASSSPGSSTPSGATGSPGSSPSASTGT |
| 743)- | GPGSSPSASTGTGPGASPGTSSTGSPGASPGTSSTGSPGSSTPSGATGSPGSSPSASTGTGPGASP |
| AG576- | GTSSTGSPGSSPSASTGTGPGTPGSGTASSSPGSSTPSGATGSIEPRSPSGSPGATRRYYLGAVEL |
| (FVIII_1686- | SWDYMQSDLGELPVDARFPPRVPKSFPFNTSVVYKKTLFVEFTVHLFNIAKPRPPWMGLLGP |
| 2332)-L- | TIQAEVYDTVVITLKNMASHPVSLHAVGVSYWKASEGAEYDDQTSQREKEDDKVFPGGSHT |
| CS-AG288 | YVWQVLKENGPMASDPLCLTYSYLSHVDLVKDLNSGLIGALLVCREGSLAKEKTQTLHKFIL |
| | LFAVFDEGKSWHSETKNSLMQDRDAASARAWPKMHTVNGYVNRSLPGLIGCHRKSVYWHV |
| | IGMGTTPEVHSIFLEGHTFLVRNHRQASLEISPITFLTAQTLLMDLGQFLLFCHISSHQHDGME |
| | AYVKVDSCPEEPQLRMKNNEEAEDYDDDLTDSEMDVVRFDDDNSPSFIQIRSVAKKHPKTW |
| | VHYIAAEEEDWDYAPLVLAPDDRSYKSQYLNNGPQRIGRKYKKVRFMAYTDETFKTREAIQ |
| | HESGILGPLLYGEVGDTLLIIFKNQASRPYNIYPHGITDVRPLYSRRLPKGVKHLKDFPILPGEIF |
| | KYKWTVTVEDGPTKSDPRCLTRYYSSFVNMERDLASGLIGPLLICYKESVDQRGNQIMSDKR |
| | NVILFSVFDENRSWYLTENIQRFLPNPAGVQLEDPEFQASNIMHSINGYVFDSLQLSVCLHEVA |
| | YWYILSIGAQTDFLSVFFSGYTFKHKMVYEDTLTLFPFSGETVFMSMENPGLWILGCHNSDFR |
| | NRGMTALLKVSSCDKNTGDYYEDSYEDISAYLLSKNNAIEPRSFSPGTPGSGTASSSPGSSTPS |
| | GATGSPGSSPSASTGTGPGSSPSASTGTGPGSSTPSGATGSPGSSTPSGATGSPGASPGTSSTGSP |
| | GASPGTSSTGSPGASPGTSSTGSPGTPGSGTASSSPGASPGTSSTGSPGASPGTSSTGSPGASPGT |
| | SSTGSPGSSPSASTGTGPGTPGSGTASSSPGASPGTSSTGSPGASPGTSSTGSPGASPGTSSTGSP |
| | GSSTPSGATGSPGSSTPSGATGSPGASPGTSSTGSPGTPGSGTASSSPGSSTPSGATGSPGSSTPS |
| | GATGSPGSSTPSGATGSPGSSPSASTGTGPGASPGTSSTGSPGASPGTSSTGSPGTPGSGTASSSP |
| | GASPGTSSTGSPGASPGTSSTGSPGASPGTSSTGSPGASPGTSSTGSPGTPGSGTASSSPGSSTPS |
| | GATGSPGTPGSGTASSSPGSSTPSGATGSPGTPGSGTASSSPGSSTPSGATGSPGSSTPSGATGSP |
| | GSSPSASTGTGPGSSPSASTGTGPGASPGTSSTGSPGTPGSGTASSSPGSSTPSGATGSPGSSPSA |
| | STGTGPGSSPSASTGTGPGASPGTSSTGSQSPRSFQKKTRHYFIAAVERLWDYGMSSSPHVLR |
| | NRAQSGSVPQFKKVVFQEFTDGSFTQPLYRGELNEHLGLLGPYIRAEVEDNIMVTFRNQASRP |
| | YSFYSSLISYEEDQRQGAEPRKNFVKPNETKTYFWKVQHHMAPTKDEFDCKAWAYFSDVDL |
| | EKDVHSGLIGPLLVCHTNTLNPAHGRQVTVQEFALFFTIFDETKSWYFTENMERNCRAPCNIQ |
| | MEDPTFKENYRFHAINGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFLVYSNKCQTPLGMAS |
| | GHIRDFQITASGQYGQWAPKLARLHYSGSINAWSTKEPFSWIKVDLLAPMIIHGIKTQGARQK |
| | FSSLYISQFIIMYSLDGKKWQTYRGNSTGTLMVFFGNVDSSGIKHNIFNPPIIARYIRLHPTHYSI |
| | RSTLRMELMGCDLNSCSMPLGMESKAISDAQITASSYFTNMFATWSPSKARLHLQGRSNAW |
| | RPQVNNPKEWLQVDFQKTMKVTGVTTQGVKSLLTSMYVKEFLISSSQDGHQWTLFFQNGKV |
| | KVFQGNQDSFTPVVNSLDPPLLTRYLRIHPQSWVHQIALRMEVLGCEAQDLYGSPGQSPRSFQ |
| | PGASPGTSSTGSPGASPGTSSTGSPGTPGSGTASSSPGSSTPSGATGSPGTPGSGTASSSPGSSTP |
| | SGATGSPGTPGSGTASSSPGSSTPSGATGSPGSSTPSGATGSPGSSPSASTGTGPGSSPSASTGTG |
| | PGASPGTSSTGSPGTPGSGTASSSPGSSTPSGATGSPGSSPSASTGTGPGSSPSASTGTGPGASPG |
| | TSSTGSPGASPGTSSTGSPGSSTPSGATGSPGSSPSASTGTGPGASPGTSSTGSPGSSPSASTGTG |
| | PGTPGSGTASSSPGSSTPSGATGS |
| SP-AG576- | MQIELSTCFFLCLLRFCFSPGTPGSGTASSSPGSSTPSGATGSPGSSPSASTGTGPGSSPSASTGT |
| CS-L- | GPGSSTPSGATGSPGSSTPSGATGSPGASPGTSSTGSPGASPGTSSTGSPGASPGTSSTGSPGTPG |
| (FVIII_1- | SGTASSSPGASPGTSSTGSPGASPGTSSTGSPGASPGTSSTGSPGSSPSASTGTGPGTPGSGTASS |
| 745)- | SPGASPGTSSTGSPGASPGTSSTGSPGASPGTSSTGSPGSSTPSGATGSPGSSTPSGATGSPGASP |
| AG288- | GTSSTGSPGTPGSGTASSSPGSSTPSGATGSPGSSTPSGATGSPGSSTPSGATGSPGSSPSASTGT |
| (FVIII_1686- | GPGASPGTSSTGSPGASPGTSSTGSPGTPGSGTASSSPGASPGTSSTGSPGASPGTSSTGSPGASP |
| 2332)-L- | GTSSTGSPGASPGTSSTGSPGTPGSGTASSSPGSSTPSGATGSPGTPGSGTASSSPGSSTPSGATG |
| CS-AE576 | SPGTPGSGTASSSPGSSTPSGATGSPGSSTPSGATGSPGSSPSASTGTGPGSSPSASTGTGPGASP |
| | GTSSTGSPGTPGSGTASSSPGSSTPSGATGSPGSSPSASTGTGPGSSPSASTGTGPGASPGTSSTG |
| | SQSPRSFQGSPGATRRYYLGAVELSWDYMQSDLGELPVDARFPPRVPKSFPFNTSVVYKKTLF |
| | VEFTVHLFNIAKPRPPWMGLLGPTIQAEVYDTVVITLKNMASHPVSLHAVGVSYWKASEGAE |
| | YDDQTSQREKEDDKVFPGGSHTYVWQVLKENGPMASDPLCLTYSYLSHVDLVKDLNSGLIG |
| | ALLVCREGSLAKEKTQTLHKFILLFAVFDEGKSWHSETKNSLMQDRDAASARAWPKMHTVN |
| | GYVNRSLPGLIGCHRKSVYWHVIGMGTTPEVHSIFLEGHTFLVRNHRQASLEISPITFLTAQTL |
| | LMDLGQFLLFCHISSHQHDGMEAYVKVDSCPEEPQLRMKNNEEAEDYDDDLTDSEMDVVRF |
| | DDDNSPSFIQIRSVAKKHPKTWVHYIAAEEEDWDYAPLVLAPDDRSYKSQYLNNGPQRIGRK |
| | YKKVRFMAYTDETFKTREAIQHESGILGPLLYGEVGDTLLIIFKNQASRPYNIYPHGITDVRPL |
| | YSRRLPKGVKHLKDFPILPGEIFKYKWTVTVEDGPTKSDPRCLTRYYSSFVNMERDLASGLIG |
| | PLLICYKESVDQRGNQIMSDKRNVILFSVFDENRSWYLTENIQRFLPNPAGVQLEDPEFQASNI |
| | MHSINGYVFDSLQLSVCLHEVAYWYILSIGAQTDFLSVFFSGYTFKHKMVYEDTLTLFPFSGE |
| | TVFMSMENPGLWILGCHNSDFRNRGMTALLKVSSCDKNTGDYYEDSYEDISAYLLSKNNAIE |
| | PRSFSQNPGASPGTSSTGSPGASPGTSSTGSPGTPGSGTASSSPGSSTPSGATGSPGTPGSGTASS |
| | SPGSSTPSGATGSPGTPGSGTASSSPGSSTPSGATGSPGSSTPSGATGSPGSSPSASTGTGPGSSP |
| | SASTGTGPGASPGTSSTGSPGTPGSGTASSSPGSSTPSGATGSPGSSPSASTGTGPGSSPSASTGT |
| | GPGASPGTSSTGSPGASPGTSSTGSPGSSTPSGATGSPGSSPSASTGTGPGASPGTSSTGSPGSSP |
| | SASTGTGPGTPGSGTASSSPGSSTPSGATGSQSPRSFQKKTRHYFIAAVERLWDYGMSSSPHVL |
| | RNRAQSGSVPQFKKVVFQEFTDGSFTQPLYRGELNEHLGLLGPYIRAEVEDNIMVTFRNQASR |
| | PYSFYSSLISYEEDQRQGAEPRKNFVKPNETKTYFWKVQHHMAPTKDEFDCKAWAYFSDVD |
| | LEKDVHSGLIGPLLVCHTNTLNPAHGRQVTVQEFALFFTIFDETKSWYFTENMERNCRAPCNI |
| | QMEDPTFKENYRFHAINGYIMDTLPGLVMAQDQRIRWYLLSMGSNENIHSIHFSGHVFTVRK |
| | KEEYKMALYNLYPGVFETVEMLPSKAGIWRVECLIGEHLHAGMSTLFLVYSNKCQTPLGMA |
| | SGHIRDFQITASGQYGQWAPKLARLHYSGSINAWSTKEPFSWIKVDLLAPMIIHGIKTQGARQ |
| | KFSSLYISQFIIMYSLDGKKWQTYRGNSTGTLMVFFGNVDSSGIKHNIFNPPIIARYIRLHPTHY |
| | SIRSTLRMELMGCDLNSCSMPLGMESKAISDAQITASSYFTNMFATWSPSKARLHLQGRSNA |
| | WRPQVNNPKEWLQVDFQKTMKVTGVTTQGVKSLLTSMYVKEFLISSSQDGHQWTLFFQNG |
| | KVKVFQGNQDSFTPVVNSLDPPLLTRYLRIHPQSWVHQIALRMEVLGCEAQDLYGSPGQSPR |
| | SFQGSPAGSPTSTEEGTSESATPESGPGTSTEPSEGSAPGSPAGSPTSTEEGTSTEPSEGSAPGTS |
| | TEPSEGSAPGTSESATPESGPGSEPATSGSETPGSEPATSGSETPGSPAGSPTSTEEGTSESATPES |
| | GPGTSTEPSEGSAPGTSTEPSEGSAPGSPAGSPTSTEEGTSTEPSEGSAPGTSTEPSEGSAPGTSE |
| | SATPESGPGTSTEPSEGSAPGTSESATPESGPGSEPATSGSETPGTSTEPSEGSAPGTSTEPSEGS |
| | APGTSESATPESGPGTSESATPESGPGSPAGSPTSTEEGTSESATPESGPGSEPATSGSETPGTSE |
| | SATPESGPGTSTEPSEGSAPGTSTEPSEGSAPGTSTEPSEGSAPGTSTEPSEGSAPGTSTEPSEGS |
| | APGTSTEPSEGSAPGSPAGSPTSTEEGTSTEPSEGSAPGTSESATPESGPGSEPATSGSETPGTSE |
| | SATPESGPGSEPATSGSETPGTSESATPESGPGTSTEPSEGSAPGTSESATPESGPGSPAGSPTST |
| | EEGSPAGSPTSTEEGSPAGSPTSTEEGTSESATPESGPGTSTEPSEGSAP |
| SP- | MQIELSTCFFLCLLRFCFSATRRYYLGAVELSWDYMQSDLGELPVDARFPPRVPKSFPFNTSV |
| (FVIII_1- | VYKKTLFVEFTVHLFNIAKPRPPWMGLLGPTIQAEVYDTVVITLKNMASHPVSLHAVGVSYW |
| 743)- | KASEGAEYDDQTSQREKEDDKVFPGGSHTYVWQVLKENGPMASDPLCLTYSYLSHVDLVK |
| AG576- | DLNSGLIGALLVCREGSLAKEKTQTLHKFILLFAVFDEGKSWHSETKNSLMQDRDAASARAW |
| (FVIII_1686- | PKMHTVNGYVNRSLPGLIGCHRKSVYWHVIGMGTTPEVHSIFLEGHTFLVRNHRQASLEISPI |
| 2332)-L- | TFLTAQTLLMDLGQFLLFCHISSHQHDGMEAYVKVDSCPEEPQLRMKNNEEAEDYDDDLTD |
| CS-AG576 | SEMDVVRFDDDNSPSFIQIRSVAKKHPKTWVHYIAAEEEDWDYAPLVLAPDDRSYKSQYLNN |
| | GPQRIGRKYKKVRFMAYTDETFKTREAIQHESGILGPLLYGEVGDTLLIIFKNQASRPYNIYPH |
| | GITDVRPLYSRRLPKGVKHLKDFPILPGEIFKYKWTVTVEDGPTKSDPRCLTRYYSSFVNMER |
| | DLASGLIGPLLICYKESVDQRGNQIMSDKRNVILFSVFDENRSWYLTENIQRFLPNPAGVQLED |
| | PEFQASNIMHSINGYVFDSLQLSVCLHEVAYWYILSIGAQTDFLSVFFSGYTFKHKMVYEDTL |
| | TLFPFSGETVFMSMENPGLWILGCHNSDFRNRGMTALLKVSSCDKNTGDYYEDSYEDISAYL |
| | LSKNNAIEPRSFSPGTPGSGTASSSPGSSTPSGATGSPGSSPSASTGTGPGSSPSASTGTGPGSST |
| | PSGATGSPGSSTPSGATGSPGASPGTSSTGSPGASPGTSSTGSPGASPGTSSTGSPGTPGSGTASS |
| | SPGASPGTSSTGSPGASPGTSSTGSPGASPGTSSTGSPGSSPSASTGTGPGTPGSGTASSSPGASP |
| | GTSSTGSPGASPGTSSTGSPGASPGTSSTGSPGSSTPSGATGSPGSSTPSGATGSPGASPGTSSTG |
| | SPGTPGSGTASSSPGSSTPSGATGSPGSSTPSGATGSPGSSTPSGATGSPGSSPSASTGTGPGASP |
| | GTSSTGSPGASPGTSSTGSPGTPGSGTASSSPGASPGTSSTGSPGASPGTSSTGSPGASPGTSSTG |
| | SPGASPGTSSTGSPGTPGSGTASSSPGSSTPSGATGSPGTPGSGTASSSPGSSTPSGATGSPGTPG |
| | SGTASSSPGSSTPSGATGSPGSSTPSGATGSPGSSPSASTGTGPGSSPSASTGTGPGASPGTSSTG |
| | SPGTPGSGTASSSPGSSTPSGATGSPGSSPSASTGTGPGSSPSASTGTGPGASPGTSSTGSQSPRS |
| | FQKKTRHYFIAAVERLWDYGMSSSPHVLRNRAQSGSVPQFKKVVFQEFTDGSFTQPLYRGEL |
| | NEHLGLLGPYIRAEVEDNIMVTFRNQASRPYSFYSSLISYEEDQRQGAEPRKNFVKPNETKTY |
| | FWKVQHHMAPTKDEFDCKAWAYFSDVDLEKDVHSGLIGPLLVCHTNTLNPAHGRQVTVQE |
| | FALFFTIFDETKSWYFTENMERNCRAPCNIQMEDPTFKENYRFHAINGYIMDTLPGLVMAQD |
| | QRIRWYLLSMGSNENIHSIHFSGHVFTVRKKEEYKMALYNLYPGVFETVEMLPSKAGIWRVE |
| | CLIGEHLHAGMSTLFLVYSNKCQTPLGMASGHIRDFQITASGQYGQWAPKLARLHYSGSINA |
| | WSTKEPFSWIKVDLLAPMIIHGIKTQGARQKFSSLYISQFIIMYSLDGKKWQTYRGNSTGTLM |
| | VFFGNVDSSGIKHNIFNPPIIARYIRLHPTHYSIRSTLRMELMGCDLNSCSMPLGMESKAISDAQ |
| | ITASSYFTNMFATWSPSKARLHLQGRSNAWRPQVNNPKEWLQVDFQKTMKVTGVTTQGVK |
| | SLLTSMYVKEFLISSSQDGHQWTLFFQNGKVKVFQGNQDSFTPVVNSLDPPLLTRYLRIHPQS |
| | WVHQIALRMEVLGCEAQDLYGSPGQSPRSFQPGTPGSGTASSSPGSSTPSGATGSPGSSPSAST |
| | GTGPGSSPSASTGTGPGSSTPSGATGSPGSSTPSGATGSPGASPGTSSTGSPGASPGTSSTGSPG |
| | ASPGTSSTGSPGTPGSGTASSSPGASPGTSSTGSPGASPGTSSTGSPGASPGTSSTGSPGSSPSAS |
| | TGTGPGTPGSGTASSSPGASPGTSSTGSPGASPGTSSTGSPGASPGTSSTGSPGSSTPSGATGSPG |
| | SSTPSGATGSPGASPGTSSTGSPGTPGSGTASSSPGSSTPSGATGSPGSSTPSGATGSPGSSTPSG |
| | ATGSPGSSPSASTGTGPGASPGTSSTGSPGASPGTSSTGSPGTPGSGTASSSPGASPGTSSTGSPG |
| | ASPGTSSTGSPGASPGTSSTGSPGASPGTSSTGSPGTPGSGTASSSPGSSTPSGATGSPGTPGSGT |
| | ASSSPGSSTPSGATGSPGTPGSGTASSSPGSSTPSGATGSPGSSTPSGATGSPGSSPSASTGTGPG |
| | SSPSASTGTGPGASPGTSSTGSPGTPGSGTASSSPGSSTPSGATGSPGSSPSASTGTGPGSSPSAS |
| | TGTGPGASPGTSSTGS |
| SP-AG288- | MQIELSTCFFLCLLRFCFSPGASPGTSSTGSPGASPGTSSTGSPGTPGSGTASSSPGSSTPSGATG |
| CS-L- | SPGTPGSGTASSSPGSSTPSGATGSPGTPGSGTASSSPGSSTPSGATGSPGSSTPSGATGSPGSSP |
| (FVIII_1- | SASTGTGPGSSPSASTGTGPGASPGTSSTGSPGTPGSGTASSSPGSSTPSGATGSPGSSPSASTGT |
| 743)- | GPGSSPSASTGTGPGASPGTSSTGSPGASPGTSSTGSPGSSTPSGATGSPGSSPSASTGTGPGASP |
| AG288- | GTSSTGSPGSSPSASTGTGPGTPGSGTASSSPGSSTPSGATGSQSPRSFQGPSGPATRRYYLGAV |
| (FVIII_1686- | ELSWDYMQSDLGELPVDARFPPRVPKSFPFNTSVVYKKTLFVEFTVHLFNIAKPRPPWMGLL |
| 2332)-L- | GPTIQAEVYDTVVITLKNMASHPVSLHAVGVSYWKASEGAEYDDQTSQREKEDDKVFPGGS |
| CS-AE288 | HTYVWQVLKENGPMASDPLCLTYSYLSHVDLVKDLNSGLIGALLVCREGSLAKEKTQTLHK |
| | FILLFAVFDEGKSWHSETKNSLMQDRDAASARAWPKMHTVNGYVNRSLPGLIGCHRKSVYW |
| | HVIGMGTTPEVHSIFLEGHTFLVRNHRQASLEISPITFLTAQTLLMDLGQFLLFCHISSHQHDG |
| | MEAYVKVDSCPEEPQLRMKNNEEAEDYDDDLTDSEMDVVRFDDDNSPSFIQIRSVAKKHPK |
| | TWVHYIAAEEEDWDYAPLVLAPDDRSYKSQYLNNGPQRIGRKYKKVRFMAYTDETFKTREA |
| | IQHESGILGPLLYGEVGDTLLIIFKNQASRPYNIYPHGITDVRPLYSRRLPKGVKHLKDFPILPGE |
| | IFKYKWTVTVEDGPTKSDPRCLTRYYSSFVNMERDLASGLIGPLLICYKESVDQRGNQIMSDK |
| | RNVILFSVFDENRSWYLTENIQRFLPNPAGVQLEDPEFQASNIMHSINGYVFDSLQLSVCLHEV |
| | AYWYILSIGAQTDFLSVFFSGYTFKHKMVYEDTLTLFPFSGETVFMSMENPGLWILGCHNSDF |
| | RNRGMTALLKVSSCDKNTGDYYEDSYEDISAYLLSKNNAIEPRSFSPGASPGTSSTGSPGASPG |
| | TSSTGSPGTPGSGTASSSPGSSTPSGATGSPGTPGSGTASSSPGSSTPSGATGSPGTPGSGTASSS |
| | PGSSTPSGATGSPGSSTPSGATGSPGSSPSASTGTGPGSSPSASTGTGPGASPGTSSTGSPGTPGS |
| | GTASSSPGSSTPSGATGSPGSSPSASTGTGPGSSPSASTGTGPGASPGTSSTGSPGASPGTSSTGS |
| | PGSSTPSGATGSPGSSPSASTGTGPGASPGTSSTGSPGSSPSASTGTGPGTPGSGTASSSPGSSTP |
| | SGATGSQSPRSFQKKTRHYFIAAVERLWDYGMSSSPHVLRNRAQSGSVPQFKKVVFQEFTDG |
| | SFTQPLYRGELNEHLGLLGPYIRAEVEDNIMVTFRNQASRPYSFYSSLISYEEDQRQGAEPRKN |
| | FVKPNETKTYFWKVQHHMAPTKDEFDCKAWAYFSDVDLEKDVHSGLIGPLLVCHTNTLNPA |
| | HGRQVTVQEFALFFTIFDETKSWYFTENMERNCRAPCNIQMEDPTFKENYRFHAINGYIMDTL |
| | PGLVMAQDQRIRWYLLSMGSNENIHSIHFSGHVFTVRKKEEYKMALYNLYPGVFETVEMLPS |
| | KAGIWRVECLIGEHLHAGMSTLFLVYSNKCQTPLGMASGHIRDFQITASGQYGQWAPKLARL |
| | HYSGSINAWSTKEPFSWIKVDLLAPMIIHGIKTQGARQKFSSLYISQFIIMYSLDGKKWQTYRG |
| | NSTGTLMVFFGNVDSSGIKHNIFNPPIIARYIRLHPTHYSIRSTLRMELMGCDLNSCSMPLGME |
| | SKAISDAQITASSYFTNMFATWSPSKARLHLQGRSNAWRPQVNNPKEWLQVDFQKTMKVTG |
| | VTTQGVKSLLTSMYVKEFLISSSQDGHQWTLFFQNGKVKVFQGNQDSFTPVVNSLDPPLLTR |
| | YLRIHPQSWVHQIALRMEVLGCEAQDLYGPSGPQSPRSFQGTSESATPESGPGSEPATSGSETP |
| | GTSESATPESGPGSEPATSGSETPGTSESATPESGPGTSTEPSEGSAPGSPAGSPTSTEEGTSESA |
| | TPESGPGSEPATSGSETPGTSESATPESGPGSPAGSPTSTEEGSPAGSPTSTEEGTSTEPSEGSAP |
| | GTSESATPESGPGTSESATPESGPGTSESATPESGPGSEPATSGSETPGSEPATSGSETPGSPAGS |
| | PTSTEEGTSTEPSEGSAPGTSTEPSEGSAPGSEPATSGSETPGTSESATPESGPGTSTEPSEGSAP |
| SP-AE576- | MQIELSTCFFLCLLRFCFSGSPAGSPTSTEEGTSESATPESGPGTSTEPSEGSAPGSPAGSPTSTEE |
| CS-L- | GTSTEPSEGSAPGTSTEPSEGSAPGTSESATPESGPGSEPATSGSETPGSEPATSGSETPGSPAGS |
| (FVIII_1- | PTSTEEGTSESATPESGPGTSTEPSEGSAPGTSTEPSEGSAPGSPAGSPTSTEEGTSTEPSEGSAP |
| 743)- | GTSTEPSEGSAPGTSESATPESGPGTSTEPSEGSAPGTSESATPESGPGSEPATSGSETPGTSTEPS |
| AG576- | EGSAPGTSTEPSEGSAPGTSESATPESGPGTSESATPESGPGSPAGSPTSTEEGTSESATPESGPG |
| (FVIII_1686- | SEPATSGSETPGTSESATPESGPGTSTEPSEGSAPGTSTEPSEGSAPGTSTEPSEGSAPGTSTEPSE |
| 2332) | GSAPGTSTEPSEGSAPGTSTEPSEGSAPGSPAGSPTSTEEGTSTEPSEGSAPGTSESATPESGPGS |
| | EPATSGSETPGTSESATPESGPGSEPATSGSETPGTSESATPESGPGTSTEPSEGSAPGTSESATP |
| | ESGPGSPAGSPTSTEEGSPAGSPTSTEEGSPAGSPTSTEEGTSESATPESGPGTSTEPSEGSAPQS |
| | PRSFQGSPGATRRYYLGAVELSWDYMQSDLGELPVDARFPPRVPKSFPFNTSVVYKKTLFVE |
| | FTVHLFNIAKPRPPWMGLLGPTIQAEVYDTVVITLKNMASHPVSLHAVGVSYWKASEGAEYD |
| | DQTSQREKEDDKVFPGGSHTYVWQVLKENGPMASDPLCLTYSYLSHVDLVKDLNSGLIGAL |
| | LVCREGSLAKEKTQTLHKFILLFAVFDEGKSWHSETKNSLMQDRDAASARAWPKMHTVNGY |
| | VNRSLPGLIGCHRKSVYWHVIGMGTTPEVHSIFLEGHTFLVRNHRQASLEISPITFLTAQTLLM |
| | DLGQFLLFCHISSHQHDGMEAYVKVDSCPEEPQLRMKNNEEAEDYDDDLTDSEMDVVRFDD |
| | DNSPSFIQIRSVAKKHPKTWVHYIAAEEEDWDYAPLVLAPDDRSYKSQYLNNGPQRIGRKYK |
| | KVRFMAYTDETFKTREAIQHESGILGPLLYGEVGDTLLIIFKNQASRPYNIYPHGITDVRPLYSR |
| | RLPKGVKHLKDFPILPGEIFKYKWTVTVEDGPTKSDPRCLTRYYSSFVNMERDLASGLIGPLLI |
| | CYKESVDQRGNQIMSDKRNVILFSVFDENRSWYLTENIQRFLPNPAGVQLEDPEFQASNIMHS |
| | INGYVFDSLQLSVCLHEVAYWYILSIGAQTDFLSVFFSGYTFKHKMVYEDTLTLFPFSGETVF |
| | MSMENPGLWILGCHNSDFRNRGMTALLKVSSCDKNTGDYYEDSYEDISAYLLSKNNAIEPRS |
| | FSPGTPGSGTASSSPGSSTPSGATGSPGSSPSASTGTGPGSSPSASTGTGPGSSTPSGATGSPGSS |
| | TPSGATGSPGASPGTSSTGSPGASPGTSSTGSPGASPGTSSTGSPGTPGSGTASSSPGASPGTSST |
| | GSPGASPGTSSTGSPGASPGTSSTGSPGSSPSASTGTGPGTPGSGTASSSPGASPGTSSTGSPGAS |
| | PGTSSTGSPGASPGTSSTGSPGSSTPSGATGSPGSSTPSGATGSPGASPGTSSTGSPGTPGSGTAS |
| | SSPGSSTPSGATGSPGSSTPSGATGSPGSSTPSGATGSPGSSPSASTGTGPGASPGTSSTGSPGAS |
| | PGTSSTGSPGTPGSGTASSSPGASPGTSSTGSPGASPGTSSTGSPGASPGTSSTGSPGASPGTSST |
| | GSPGTPGSGTASSSPGSSTPSGATGSPGTPGSGTASSSPGSSTPSGATGSPGTPGSGTASSSPGSS |
| | TPSGATGSPGSSTPSGATGSPGSSPSASTGTGPGSSPSASTGTGPGASPGTSSTGSPGTPGSGTAS |
| | SSPGSSTPSGATGSPGSSPSASTGTGPGSSPSASTGTGPGASPGTSSTGSQSPRSFQKKTRHYFIA |
| | AVERLWDYGMSSSPHVLRNRAQSGSVPQFKKVVFQEFTDGSFTQPLYRGELNEHLGLLGPYI |
| | RAEVEDNIMVTFRNQASRPYSFYSSLISYEEDQRQGAEPRKNFVKPNETKTYFWKVQHHMAP |
| | TKDEFDCKAWAYFSDVDLEKDVHSGLIGPLLVCHTNTLNPAHGRQVTVQEFALFFTIFDETKS |
| | WYFTENMERNCRAPCNIQMEDPTFKENYRFHAINGYIMDTLPGLVMAQDQRIRWYLLSMGS |
| | NENIHSIHFSGHVFTVRKKEEYKMALYNLYPGVFETVEMLPSKAGIWRVECLIGEHLHAGMS |
| | TLFLVYSNKCQTPLGMASGHIRDFQITASGQYGQWAPKLARLHYSGSINAWSTKEPFSWIKV |
| | DLLAPMIIHGIKTQGARQKFSSLYISQFIIMYSLDGKKWQTYRGNSTGTLMVFFGNVDSSGIKH |
| | NIFNPPIIARYIRLHPTHYSIRSTLRMELMGCDLNSCSMPLGMESKAISDAQITASSYFTNMFAT |
| | WSPSKARLHLQGRSNAWRPQVNNPKEWLQVDFQKTMKVTGVTTQGVKSLLTSMYVKEFLI |
| | SSSQDGHQWTLFFQNGKVKVFQGNQDSFTPVVNSLDPPLLTRYLRIHPQSWVHQIALRMEVL |
| | GCEAQDLY |
| FVIII | ATRRYYLGAVELSWDYMQSDLGELPVDARFPPRVPKSFPFNTSVVYKKTLFVEFTVHLFNIA |
| BDD2 | KPRPPWMGLLGPTIQAEVYDTVVITLKNMASHPVSLHAVGVSYWKASEGAEYDDQTSQREK |
| S367- | EDDKVFPGGSHTYVWQVLKENGPMASDPLCLTYSYLSHVDLVKDLNSGLIGALLVCREGSL |
| FXIa- | AKEKTQTLHKFILLFAVFDEGKSWHSETKNSLMQDRDAASARAWPKMHTVNGYVNSSLPGL |
| AE42- | IGCHRKSVYWHVIGMGTTPEVHSIFLEGHTFLVRNHRQASLEISPITFLTAQTLLMDLGQFLLF |
| F368- | CHISSHQHDGMEAYVKVDSCPEEPQLRMKNNEEAEDYDDDLTDSEMDVVRFDDDNSPSKLT |
| Y2332- | RAETGEPSEGSAPGSPAGSPTSTEEGTSESATPESGPGSEPATSGSGFIQIRSVAKKHPKTWVHY |
| FXIa- | IAAEEEDWDYAPLVLAPDDRSYKSQYLNNGPQRIGRKYKKVRFMAYTDETFKTREAIQHESG |
| AE864 | ILGPLLYGEVGDTLLIIFKNQASRPYNIYPHGITDVRPLYSRRLPKGVKHLKDFPILPGEIFKYK |
| | WTVTVEDGPTKSDPRCLTRYYSSFVNMERDLASGLIGPLLICYKESVDQRGNQIMSDKRNVIL |
| | FSVFDENRSWYLTENIQRFLPNPAGVQLEDPEFQASNIMHSINGYVFDSLQLSVCLHEVAYWY |
| | ILSIGAQTDFLSVFFSGYTFKHKMVYEDTLTLFPFSGETVFMSMENPGLWILGCHNSDFRNRG |
| | MTALLKVSSCDKNTGDYYEDSYEDISAYLLSKNNAIEPRSFSQNPPVLKRHQREITRTTLQSD |
| | QEEIDYDDTISVEMKKEDFDIYDEDENQSPRSFQKKTRHYFIAAVERLWDYGMSSSPHVLRNR |
| | AQSGSVPQFKKVVFQEFTDGSFTQPLYRGELNEHLGLLGPYIRAEVEDNIMVTFRNQASRPYS |
| | FYSSLISYEEDQRQGAEPRKNFVKPNETKTYFWKVQHHMAPTKDEFDCKAWAYFSDVDLEK |
| | DVHSGLIGPLLVCHTNTLNPAHGRQVTVQEFALFFTIFDETKSWYFTENMERNCRAPCNIQME |
| | DPTFKENYRFHAINGYIMDTLPGLVMAQDQRIRWYLLSMGSNENIHSIHFSGHVFTVRKKEEY |
| | KMALYNLYPGVFETVEMLPSKAGIWRVECLIGEHLHAGMSTLFLVYSNKCQTPLGMASGHIR |
| | DFQITASGQYGQWAPKLARLHYSGSINAWSTKEPFSWIKVDLLAPMIIHGIKTQGARQKFSSL |
| | YISQFIIMYSLDGKKWQTYRGNSTGTLMVFFGNVDSSGIKHNIFNPPIIARYIRLHPTHYSIRSTL |
| | RMELMGCDLNSCSMPLGMESKAISDAQITASSYFTNMFATWTPSKARLHLQGRSNAWRPQV |
| | NNPKEWLQVDFQKTMKVTGVTTQGVKSLLTSMYVKEFLISSSQDGHQWTLFFQNGKVKVFQ |
| | GNQDSFTPVVNSLDPPLLTRYLRIHPQSWVHQIALRMEVLGCEAQDLYKLTRAETGGSPAGSP |
| | TSTEEGTSESATPESGPGTSTEPSEGSAPGSPAGSPTSTEEGTSTEPSEGSAPGTSTEPSEGSAPG |
| | TSESATPESGPGSEPATSGSETPGSEPATSGSETPGSPAGSPTSTEEGTSESATPESGPGTSTEPSE |
| | GSAPGTSTEPSEGSAPGSPAGSPTSTEEGTSTEPSEGSAPGTSTEPSEGSAPGTSESATPESGPGT |
| | STEPSEGSAPGTSESATPESGPGSEPATSGSETPGTSTEPSEGSAPGTSTEPSEGSAPGTSESATPE |
| | SGPGTSESATPESGPGSPAGSPTSTEEGTSESATPESGPGSEPATSGSETPGTSESATPESGPGTS |
| | TEPSEGSAPGTSTEPSEGSAPGTSTEPSEGSAPGTSTEPSEGSAPGTSTEPSEGSAPGTSTEPSEGS |
| | APGSPAGSPTSTEEGTSTEPSEGSAPGTSESATPESGPGSEPATSGSETPGTSESATPESGPGSEP |
| | ATSGSETPGTSESATPESGPGTSTEPSEGSAPGTSESATPESGPGSPAGSPTSTEEGSPAGSPTST |
| | EEGSPAGSPTSTEEGTSESATPESGPGTSTEPSEGSAPGTSESATPESGPGSEPATSGSETPGTSE |
| | SATPESGPGSEPATSGSETPGTSESATPESGPGTSTEPSEGSAPGSPAGSPTSTEEGTSESATPES |
| | GPGSEPATSGSETPGTSESATPESGPGSPAGSPTSTEEGSPAGSPTSTEEGTSTEPSEGSAPGTSE |
| | SATPESGPGTSESATPESGPGTSESATPESGPGSEPATSGSETPGSEPATSGSETPGSPAGSPTST |
| | EEGTSTEPSEGSAPGTSTEPSEGSAPGSEPATSGSETPGTSESATPESGPGTSTEPSEGSAP |
| FVIII | ATRRYYLGAVELSWDYMQSDLGELPVDARFPPRVPKSFPFNTSVVYKKTLFVEFTVHLFNIA |
| BDD2 | KPRPPWMGLLGPTIQAEVYDTVVITLKNMASHPVSLHAVGVSYWKASEGAEYDDQTSQREK |
| N745- | EDDKVFPGGSHTYVWQVLKENGPMASDPLCLTYSYLSHVDLVKDLNSGLIGALLVCREGSL |
| FIXa- | AKEKTQTLHKFILLFAVFDEGKSWHSETKNSLMQDRDAASARAWPKMHTVNGYVNRSLPGL |
| AG288- | IGCHRKSVYWHVIGMGTTPEVHSIFLEGHTFLVRNHRQASLEISPITFLTAQTLLMDLGQFLLF |
| FIXa- | CHISSHQHDGMEAYVKVDSCPEEPQLRMKNNEEAEDYDDDLTDSEMDVVRFDDDNSPSFIQI |
| P1640- | RSVAKKHPKTWVHYIAAEEEDWDYAPLVLAPDDRSYKSQYLNNGPQRIGRKYKKVRFMAY |
| Y2332- | TDETFKTREAIQHESGILGPLLYGEVGDTLLIIFKNQASRPYNIYPHGITDVRPLYSRRLPKGVK |
| FIXa- | HLKDFPILPGEIFKYKWTVTVEDGPTKSDPRCLTRYYSSFVNMERDLASGLIGPLLICYKESVD |
| AG864 | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPLGR |
| | IVGGPGASPGTSSTGSPGASPGTSSTGSPGTPGSGTASSSPGSSTPSGATGSPGTPGSGTASSSPG |
| | SSTPSGATGSPGTPGSGTASSSPGSSTPSGATGSPGSSTPSGATGSPGSSPSASTGTGPGSSPSAS |
| | TGTGPGASPGTSSTGSPGTPGSGTASSSPGSSTPSGATGSPGSSPSASTGTGPGSSPSASTGTGPG |
| | ASPGTSSTGSPGASPGTSSTGSPGSSTPSGATGSPGSSPSASTGTGPGASPGTSSTGSPGSSPSAS |
| | TGTGPGTPGSGTASSSPGSSTPSGATGSGPLGRIVGGPPVLKRHQREITRTTLQSDQEEIDYDDT |
| | ISVEMKKEDFDIYDEDENQSPRSFQKKTRHYFIAAVERLWDYGMSSSPHVLRNRAQSGSVPQ |
| | FKKVVFQEFTDGSFTQPLYRGELNEHLGLLGPYIRAEVEDNIMVTFRNQASRPYSFYSSLISYE |
| | EDQRQGAEPRKNFVKPNETKTYFWKVQHHMAPTKDEFDCKAWAYFSDVDLEKDVHSGLIG |
| | PLLVCHTNTLNPAHGRQVTVQEFALFFTIFDETKSWYFTENMERNCRAPCNIQMEDPTFKEN |
| | YRFHAINGYIMDTLPGLVMAQDQRIRWYLLSMGSNENIHSIHFSGHVFTVRKKEEYKMALYN |
| | LYPGVFETVEMLPSKAGIWRVECLIGEHLHAGMSTLFLVYSNKCQTPLGMASGHIRDFQITAS |
| | GQYGQWAPKLARLHYSGSINAWSTKEPFSWIKVDLLAPMIIHGIKTQGARQKFSSLYISQFIIM |
| | YSLDGKKWQTYRGNSTGTLMVFFGNVDSSGIKHNIFNPPIIARYIRLHPTHYSIRSTLRMELMG |
| | CDLNSCSMPLGMESKAISDAQITASSYFTNMFATWSPSKARLHLQGRSNAWRPQVNNPKEW |
| | LQVDFQKTMKVTGVTTQGVKSLLTSMYVKEFLISSSQDGHQWTLFFQNGKVKVFQGNQDSF |
| | TPVVNSLDPPLLTRYLRIHPQSWVHQIALRMEVLGCEAQDLYPLGRIVGGGASPGTSSTGSPG |
| | SSPSASTGTGPGSSPSASTGTGPGTPGSGTASSSPGSSTPSGATGSPGSSPSASTGTGPGASPGTS |
| | STGSPGTPGSGTASSSPGSSTPSGATGSPGTPGSGTASSSPGASPGTSSTGSPGASPGTSSTGSPG |
| | TPGSGTASSSPGSSTPSGATGSPGASPGTSSTGSPGTPGSGTASSSPGSSTPSGATGSPGSSPSAS |
| | TGTGPGSSPSASTGTGPGSSTPSGATGSPGSSTPSGATGSPGASPGTSSTGSPGASPGTSSTGSPG |
| | ASPGTSSTGSPGTPGSGTASSSPGASPGTSSTGSPGASPGTSSTGSPGASPGTSSTGSPGSSPSAS |
| | TGTGPGTPGSGTASSSPGASPGTSSTGSPGASPGTSSTGSPGASPGTSSTGSPGSSTPSGATGSPG |
| | SSTPSGATGSPGASPGTSSTGSPGTPGSGTASSSPGSSTPSGATGSPGSSTPSGATGSPGSSTPSG |
| | ATGSPGSSPSASTGTGPGASPGTSSTGSPGASPGTSSTGSPGTPGSGTASSSPGASPGTSSTGSPG |
| | ASPGTSSTGSPGASPGTSSTGSPGASPGTSSTGSPGTPGSGTASSSPGSSTPSGATGSPGTPGSGT |
| | ASSSPGSSTPSGATGSPGTPGSGTASSSPGSSTPSGATGSPGSSTPSGATGSPGSSPSASTGTGPG |
| | SSPSASTGTGPGASPGTSSTGSPGTPGSGTASSSPGSSTPSGATGSPGSSPSASTGTGPGSSPSAS |
| | TGTGPGASPGTSSTGSPGASPGTSSTGSPGSSTPSGATGSPGSSPSASTGTGPGASPGTSSTGSPG |
| | SSPSASTGTGPGTPGSGTASSSPGSSTPSGATGSPGSSTPSGATGSPGASPGTSSTGSP |
| FVIII | ATRRYYLGAVELSWDYMQSDLGELPVDARFPPRVPKSFPFNTSVVYKKTLFVEFTVHLFNIA |
| BDD2 | KPRPPWMGLLGPTIQAEVYDTVVITLKNMASHPVSLHAVGVSYWKASEGAEYDDQTSQREK |
| V128- | EDDKVLQVRIVGGGAPSPSASTGTGPGTPGSGTASSSPGSSTPSGATGSPGPSGPGLQVRIVGG |
| FVIIa- | FPGGSHTYVWQVLKENGPMASDPLCLTYSYLSHVDLVKDLNSGLIGALLVCREGSLAKEKTQ |
| AG42- | TLHKFILLFAVFDEGKSWHSETKNSLMQDRDAASARAWPKMHTVNGYVNRSLPGLIGCHRK |
| FVIIa- | SVYWHVIGMGTTPEVHSIFLEGHTFLVRNHRQASLEISPITFLTAQTLLMDLGQFLLFCHISSH |
| G2044- | QHDGMEAYVKVDSCPEEPQLRMKNNEEAEDYDDDLTDSEMDVVRFDDDNSPSFIQIRSVAK |
| FVIIa- | KHPKTWVHYIAAEEEDWDYAPLVLAPDDRSYKSQYLNNGPQRIGRKYKKVRFMAYTDETF |
| AG144- | KTREAIQHESGILGPLLYGEVGDTLLIIFKNQASRPYNIYPHGITDVRPLYSRRLPKGVKHLKDF |
| Y2332- | PILPGEIFKYKWTVTVEDGPTKSDPRCLTRYYSSFVNMERDLASGLIGPLLICYKESVDQRGNQ |
| FVIIa- | IMSDKRNVILFSVFDENRSWYLTENIQRFLPNPAGVQLEDPEFQASNIMHSINGYVFDSLQLSV |
| AG576 | CLHEVAYWYILSIGAQTDFLSVFFSGYTFKHKMVYEDTLTLFPFSGETVFMSMENPGLWILGC |
| | HNSDFRNRGMTALLKVSSCDKNTGDYYEDSYEDISAYLLSKNNAIEPRSFSQNPPVLKRHQRE |
| | ITRTTLQSDQEEIDYDDTISVEMKKEDFDIYDEDENQSPRSFQKKTRHYFIAAVERLWDYGMS |
| | SSPHVLRNRAQSGSVPQFKKVVFQEFTDGSFTQPLYRGELNEHLGLLGPYIRAEVEDNIMVTF |
| | RNQASRPYSFYSSLISYEEDQRQGAEPRKNFVKPNETKTYFWKVQHHMAPTKDEFDCKAWA |
| | YFSDVDLEKDVHSGLIGPLLVCHTNTLNPAHGRQVTVQEFALFFTIFDETKSWYFTENMERNC |
| | RAPCNIQMEDPTFKENYRFHAINGYIMDTLPGLVMAQDQRIRWYLLSMGSNENIHSIHFSGHV |
| | FTVRKKEEYKMALYNLYPGVFETVEMLPSKAGIWRVECLIGEHLHAGMSTLFLVYSNKCQTP |
| | LGMASGHIRDFQITASGQYGLQVRIVGGSGTASSSPGSSTPSGATGSPGTPGSGTASSSPGSSTP |
| | SGATGSPGSSTPSGATGSPGSSPSASTGTGPGSSPSASTGTGPGASPGTSSTGSPGTPGSGTASSS |
| | PGSSTPSGATGSPGSSPSASTGTGPGSSPSASTGTGPGASPGLQVRIVGGQWAPKLARLHYSGS |
| | INAWSTKEPFSWIKVDLLAPMIIHGIKTQGARQKFSSLYISQFIIMYSLDGKKWQTYRGNSTGT |
| | LMVFFGNVDSSGIKHNIFNPPIIARYIRLHPTHYSIRSTLRMELMGCDLNSCSMPLGMESKAISD |
| | AQITASSYFTNMFATWSPSKARLHLQGRSNAWRPQVNNPKEWLQVDFQKTMKVTGVTTQG |
| | VKSLLTSMYVKEFLISSSQDGHQWTLFFQNGKVKVFQGNQDSFTPVVNSLDPPLLTRYLRIHP |
| | QSWVHQIALRMEVLGCEAQDLYLQVRIVGGPGTPGSGTASSSPGSSTPSGATGSPGSSPSAST |
| | GTGPGSSPSASTGTGPGSSTPSGATGSPGSSTPSGATGSPGASPGTSSTGSPGASPGTSSTGSPG |
| | ASPGTSSTGSPGTPGSGTASSSPGASPGTSSTGSPGASPGTSSTGSPGASPGTSSTGSPGSSPSAS |
| | TGTGPGTPGSGTASSSPGASPGTSSTGSPGASPGTSSTGSPGASPGTSSTGSPGSSTPSGATGSPG |
| | SSTPSGATGSPGASPGTSSTGSPGTPGSGTASSSPGSSTPSGATGSPGSSTPSGATGSPGSSTPSG |
| | ATGSPGSSPSASTGTGPGASPGTSSTGSPGASPGTSSTGSPGTPGSGTASSSPGASPGTSSTGSPG |
| | ASPGTSSTGSPGASPGTSSTGSPGASPGTSSTGSPGTPGSGTASSSPGSSTPSGATGSPGTPGSGT |
| | ASSSPGSSTPSGATGSPGTPGSGTASSSPGSSTPSGATGSPGSSTPSGATGSPGSSPSASTGTGPG |
| | SSPSASTGTGPGASPGTSSTGSPGTPGSGTASSSPGSSTPSGATGSPGSSPSASTGTGPGSSPSAS |
| | TGTGPGASPGTSSTGS |
| AE864- | GSPAGSPTSTEEGTSESATPESGPGTSTEPSEGSAPGSPAGSPTSTEEGTSTEPSEGSAPGTSTEPS |
| FVIII- | EGSAPGTSESATPESGPGSEPATSGSETPGSEPATSGSETPGSPAGSPTSTEEGTSESATPESGPG |
| Thrombin- | TSTEPSEGSAPGTSTEPSEGSAPGSPAGSPTSTEEGTSTEPSEGSAPGTSTEPSEGSAPGTSESATP |
| AE144 | ESGPGTSTEPSEGSAPGTSESATPESGPGSEPATSGSETPGTSTEPSEGSAPGTSTEPSEGSAPGT |
| | SESATPESGPGTSESATPESGPGSPAGSPTSTEEGTSESATPESGPGSEPATSGSETPGTSESATPE |
| | SGPGTSTEPSEGSAPGTSTEPSEGSAPGTSTEPSEGSAPGTSTEPSEGSAPGTSTEPSEGSAPGTS |
| | TEPSEGSAPGSPAGSPTSTEEGTSTEPSEGSAPGTSESATPESGPGSEPATSGSETPGTSESATPES |
| | GPGSEPATSGSETPGTSESATPESGPGTSTEPSEGSAPGTSESATPESGPGSPAGSPTSTEEGSPA |
| | GSPTSTEEGSPAGSPTSTEEGTSESATPESGPGTSTEPSEGSAPGTSESATPESGPGSEPATSGSE |
| | TPGTSESATPESGPGSEPATSGSETPGTSESATPESGPGTSTEPSEGSAPGSPAGSPTSTEEGTSES |
| | ATPESGPGSEPATSGSETPGTSESATPESGPGSPAGSPTSTEEGSPAGSPTSTEEGTSTEPSEGSA |
| | PGTSESATPESGPGTSESATPESGPGTSESATPESGPGSEPATSGSETPGSEPATSGSETPGSPAG |
| | SPTSTEEGTSTEPSEGSAPGTSTEPSEGSAPGSEPATSGSETPGTSESATPESGPGTSTEPSEGSAP |
| | GATRRYYLGAVELSWDYMQSDLGELPVDARFPPRVPKSFPFNTSVVYKKTLFVEFTDHLFNI |
| | AKPRPPWMGLLGPTIQAEVYDTVVITLKNMASHPVSLHAVGVSYWKASEGAEYDDQTSQRE |
| | KEDDKVFPGGSHTYVWQVLKENGPMASDPLCLTYSYLSHVDLVKDLNSGLIGALLVCREGS |
| | LAKEKTQTLHKFILLFAVFDEGKSWHSETKNSLMQDRDAASARAWPKMHTVNGYVNRSLPG |
| | LIGCHRKSVYWHVIGMGTTPEVHSIFLEGHTFLVRNHRQASLEISPITFLTAQTLLMDLGQFLL |
| | FCHISSHQHDGMEAYVKVDSCPEEPQLRMKNNEEAEDYDDDLTDSEMDVVRFDDDNSPSFIQ |
| | IRSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNSRHP |
| | STRQKQFNATTIPENDIEKTDPWFAHRTPMPKIQNVSSSDLLMLLRQSPTPHGLSLSDLQEAKY |
| | ETFSDDPSPGAIDSNNSLSEMTHFRPQLHHSGDMVFTPESGLQLRLNEKLGTTAATELKKLDF |
| | KVSSTSNNLISTIPSDNLAAGTDNTSSLGPPSMPVHYDSQLDTTLFGKKSSPLTESGGPLSLSEE |
| | NNDSKLLESGLMNSQESSWGKNVSSTESGRLFKGKRAHGPALLTKDNALFKVSISLLKTNKT |
| | SNNSATNRKTHIDGPSLLIENSPSVWQNILESDTEFKKVTPLIHDRMLMDKNATALRLNHMSN |
| | KTTSSKNMEMVQQKKEGPIPPDAQNPDMSFFKMLFLPESARWIQRTHGKNSLNSGQGPSPKQ |
| | LVSLGPEKSVEGQNFLSEKNKVVVGKGEFTKDVGLKEMVFPSSRNLFLTNLDNLHENNTHNQ |
| | EKKIQEEIEKKETLIQENVVLPQIHTVTGTKNFMKNLFLLSTRQNVEGSYDGAYAPVLQDFRS |
| | LNDSTNRTKKHTAHFSKKGEEENLEGLGNQTKQIVEKYACTTRISPNTSQQNFVTQRSKRAL |
| | KQFRLPLEETELEKRIIVDDTSTQWSKNMKHLTPSTLTQIDYNEKEKGAITQSPLSDCLTRSHSI |
| | PQANRSPLPIAKVSSFPSIRPIYLTRVLFQDNSSHLPAASYRKKDSGVQESSHFLQGAKKNNLS |
| | LAILTLEMTGDQREVGSLGTSATNSVTYKKVENTVLPKPDLPKTSGKVELLPKVHIYQKDLFP |
| | TETSNGSPGHLDLVEGSLLQGTEGAIKWNEANRPGKVPFLRVATESSAKTPSKLLDPLAWDN |
| | HYGTQIPKEEWKSQEKSPEKTAFKKKDTILSLNACESNHAIAAINEGQNKPEIEVTWAKQGRT |
| | ERLCSQNPPVLKRHQREITRTTLQSDQEEIDYDDTISVEMKKEDFDIYDEDENQSPRSFQKKTR |
| | HYFIAAVERLWDYGMSSSPHVLRNRAQSGSVPQFKKVVFQEFTDGSFTQPLYRGELNEHLGL |
| | LGPYIRAEVEDNIMVTFRNQASRPYSFYSSLISYEEDQRQGAEPRKNFVKPNETKTYFWKVQH |
| | HMAPTKDEFDCKAWAYFSDVDLEKDVHSGLIGPLLVCHTNTLNPAHGRQVTVQEFALFFTIF |
| | DETKSWYFTENMERNCRAPCNIQMEDPTFKENYRFHAINGYIMDTLPGLVMAQDQRIRWYL |
| | LSMGSNENIHSIHFSGHVFTVRKKEEYKMALYNLYPGVFETVEMLPSKAGIWRVECLIGEHLH |
| | AGMSTLFLVYSNKCQTPLGMASGHIRDFQITASGQYGQWAPKLARLHYSGSINAWSTKEPFS |
| | WIKVDLLAPMIIHGIKTQGARQKFSSLYISQFIIMYSLDGKKWQTYRGNSTGTLMVFFGNVDS |
| | SGIKHNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTSMYV |
| | KEFLISSSQDGHQWTLFFQNGKVKVFQGNQDSFTPVVNSLDPPLLTRYLRIHPQSWVHQIALR |
| | MEVLGCEAQDLYGLTPRSLLVGGSEPATSGSETPGTSESATPESGPGSEPATSGSETPGSPAGS |
| | PTSTEEGTSTEPSEGSAPGSEPATSGSETPGSEPATSGSETPGSEPATSGSETPGTSTEPSEGSAP |
| | GTSESATPESGPGSEPATSGSETPGTSTEPSEGSAP |
| FVIII | ATRRYYLGAVELSWDYMQSDLGELPVDARFPPRVPKSFPFNTSVVYKKTLFVEFTVHLFNIA |
| BDD3- | KPRPPWMGLLGPTIQAEVYDTVVITLKNMASHPVSLHAVGVSYWKASEGAEYDDQTSQREK |
| FXIIa- | EDDKVFPGGSHTYVWQVLKENGPMASDPLCLTYSYLSHVDLVKDLNSGLIGALLVCREGSL |
| AE144 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQGEITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYGTMTRIVGGGGSEPATSGSETPGTSESATPESGPGSEPATSGSETPGSPAGSPTSTEEGTS |
| | TEPSEGSAPGSEPATSGSETPGSEPATSGSETPGSEPATSGSETPGTSTEPSEGSAPGTSESATPES |
| | GPGSEPATSGSETPGTSTEPSEGSAP |
| FVIII | ATRRYYLGAVELSWDYMQSDLGELPVDARFPPRVPKSFPFNTSVVYKKTLFVEFTVHLFNIA |
| BDD3- | KPRPPWMGLLGPTIQAEVYDTVVITLKNMASHPVSLHAVGVSYWKASEGAEYDDQTSQREK |
| Elastase- | EDDKVFPGGSHTYVWQVLKENGPMASDPLCLTYSYLSHVDLVKDLNSGLIGALLVCREGSL |
| AE144 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQGEITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYGGGSEPATSGSETPGTSESATPESGPGSEPATSGSETPGSPAGSPTSTEEGTSTEPSEGSAP |
| | GSEPATSGSETPGSEPATSGSETPGSEPATSGSETPGTSTEPSEGSAPGTSESATPESGPGSEPAT |
| | SGSETPGTSTEPSEGSAP |
| FVIII | ATRRYYLGAVELSWDYMQSDLGELPVDARFPPRVPKSFPFNTSVVYKKTLFVEFTVHLFNIA |
| BDD3- | KPRPPWMGLLGPTIQAEVYDTVVITLKNMASHPVSLHAVGVSYWKASEGAEYDDQTSQREK |
| FXIa- | EDDKVFPGGSHTYVWQVLKENGPMASDPLCLTYSYLSHVDLVKDLNSGLIGALLVCREGSL |
| AE144 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQGEITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYGKLTRAETGGSEPATSGSETPGTSESATPESGPGSEPATSGSETPGSPAGSPTSTEEGTST |
| | EPSEGSAPGSEPATSGSETPGSEPATSGSETPGSEPATSGSETPGTSTEPSEGSAPGTSESATPES |
| | GPGSEPATSGSETPGTSTEPSEGSAP |
| FVIII | ATRRYYLGAVELSWDYMQSDLGELPVDARFPPRVPKSFPFNTSVVYKKTLFVEFTVHLFNIA |
| BDD3- | KPRPPWMGLLGPTIQAEVYDTVVITLKNMASHPVSLHAVGVSYWKASEGAEYDDQTSQREK |
| Thrombin- | EDDKVFPGGSHTYVWQVLKENGPMASDPLCLTYSYLSHVDLVKDLNSGLIGALLVCREGSL |
| AE144 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQGEITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYGLTPRSLLVGGSEPATSGSETPGTSESATPESGPGSEPATSGSETPGSPAGSPTSTEEGTS |
| | TEPSEGSAPGSEPATSGSETPGSEPATSGSETPGSEPATSGSETPGTSTEPSEGSAPGTSESATPES |
| | GPGSEPATSGSETPGTSTEPSEGSAP |
| AE144- | GSEPATSGSETPGTSESATPESGPGSEPATSGSETPGSPAGSPTSTEEGTSTEPSEGSAPGSEPAT |
| FVIII | SGSETPGSEPATSGSETPGSEPATSGSETPGTSTEPSEGSAPGTSESATPESGPGSEPATSGSETP |
| BDD2- | GTSTEPSEGSAPGATRRYYLGAVELSWDYMQSDLGELPVDARFPPRVPKSFPFNTSVVYKKT |
| MMP-17- | LFVEFTVHLFNIAKPRPPWMGLLGPTIQAEVYDTVVITLKNMASHPVSLHAVGVSYWKASEG |
| AE864 | AEYDDQTSQREKEDDKVFPGGSHTYVWQVLKENGPMASDPLCLTYSYLSHVDLVKDLNSGL |
| | IGALLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFLTAQ |
| | TLLMDLGQFLLFCHISSHQHDGMEAYVKVDSCPEEPQLRMKNNEEAEDYDDDLTDSEMDVV |
| | RFDDDNSPSFIQIRSVAKKHPKTWVHYIAAEEEDWDYAPLVLAPDDRSYKSQYLNNGPQRIG |
| | RKYKKVRFMAYTDETFKTREAIQHESGILGPLLYGEVGDTLLIIFKNQASRPYNIYPHGITDVR |
| | PLYSRRLPKGVKHLKDFPILPGEIFKYKWTVTVEDGPTKSDPRCLTRYYSSFVNMERDLASGL |
| | IGPLLICYKESVDQRGNQIMSDKRNVILFSVFDENRSWYLTENIQRFLPNPAGVQLEDPEFQAS |
| | NIMHSINGYVFDSLQLSVCLHEVAYWYILSIGAQTDFLSVFFSGYTFKHKMVYEDTLTLFPFS |
| | GETVFMSMENPGLWILGCHNSDFRNRGMTALLKVSSCDKNTGDYYEDSYEDISAYLLSKNN |
| | AIEPRSFSQNPPVLKRHQREITRTTLQSDQEEIDYDDTISVEMKKEDFDIYDEDENQSPRSFQKK |
| | TRHYFIAAVERLWDYGMSSSPHVLRNRAQSGSVPQFKKVVFQEFTDGSFTQPLYRGELNEHL |
| | GLLGPYIRAEVEDNIMVTFRNQASRPYSFYSSLISYEEDQRQGAEPRKNFVKPNETKTYFWKV |
| | QHHMAPTKDEFDCKAWAYFSDVDLEKDVHSGLIGPLLVCHTNTLNPAHGRQVTVQEFALFF |
| | TIFDETKSWYFTENMERNCRAPCNIQMEDPTFKENYRFHAINGYIMDTLPGLVMAQDQRIRW |
| | YLLSMGSNENIHSIHFSGHVFTVRKKEEYKMALYNLYPGVFETVEMLPSKAGIWRVECLIGEH |
| | LHAGMSTLFLVYSNKCQTPLGMASGHIRDFQITASGQYGQWAPKLARLHYSGSINAWSTKEP |
| | FSWIKVDLLAPMIIHGIKTQGARQKFSSLYISQFIIMYSLDGKKWQTYRGNSTGTLMVFFGNV |
| | DSSGIKHNIFNPPIIARYIRLHPTHYSIRSTLRMELMGCDLNSCSMPLGMESKAISDAQITASSYF |
| | TNMFATWSPSKARLHLQGRSNAWRPQVNNPKEWLQVDFQKTMKVTGVTTQGVKSLLTSM |
| | YVKEFLISSSQDGHQWTLFFQNGKVKVFQGNQDSFTPVVNSLDPPLLTRYLRIHPQSWVHQIA |
| | LRMEVLGCEAQDLYGAPLGLRLRGGSPAGSPTSTEEGTSESATPESGPGTSTEPSEGSAPGSPA |
| | GSPTSTEEGTSTEPSEGSAPGTSTEPSEGSAPGTSESATPESGPGSEPATSGSETPGSEPATSGSE |
| | TPGSPAGSPTSTEEGTSESATPESGPGTSTEPSEGSAPGTSTEPSEGSAPGSPAGSPTSTEEGTST |
| | EPSEGSAPGTSTEPSEGSAPGTSESATPESGPGTSTEPSEGSAPGTSESATPESGPGSEPATSGSE |
| | TPGTSTEPSEGSAPGTSTEPSEGSAPGTSESATPESGPGTSESATPESGPGSPAGSPTSTEEGTSES |
| | ATPESGPGSEPATSGSETPGTSESATPESGPGTSTEPSEGSAPGTSTEPSEGSAPGTSTEPSEGSA |
| | PGTSTEPSEGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTSTEPSEGSA |
| | PGTSESATPESGPGSPAGSPTSTEEGSPAGSPTSTEEGSPAGSPTSTEEGTSESATPESGPGTSTEP |
| | SEGSAPGTSESATPESGPGSEPATSGSETPGTSESATPESGPGSEPATSGSETPGTSESATPESGP |
| | GTSTEPSEGSAPGSPAGSPTSTEEGTSESATPESGPGSEPATSGSETPGTSESATPESGPGSPAGS |
| | PTSTEEGSPAGSPTSTEEGTSTEPSEGSAPGTSESATPESGPGTSESATPESGPGTSESATPESGP |
| | GSEPATSGSETPGSEPATSGSETPGSPAGSPTSTEEGTSTEPSEGSAPGTSTEPSEGSAPGSEPAT |
| | SGSETPGTSESATPESGPGTSTEPSEGSAP |
| AE144- | GSEPATSGSETPGTSESATPESGPGSEPATSGSETPGSPAGSPTSTEEGTSTEPSEGSAPGSEPAT |
| FVIII | SGSETPGSEPATSGSETPGSEPATSGSETPGTSTEPSEGSAPGTSESATPESGPGSEPATSGSETP |
| BDD2- | GTSTEPSEGSAPGATRRYYLGAVELSWDYMQSDLGELPVDARFPPRVPKSFPFNTSVVYKKT |
| FXIIa- | LFVEFTVHLFNIAKPRPPWMGLLGPTIQAEVYDTVVITLKNMASHPVSLHAVGVSYWKASEG |
| AE864 | AEYDDQTSQREKEDDKVFPGGSHTYVWQVLKENGPMASDPLCLTYSYLSHVDLVKDLNSGL |
| | IGALLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFLTAQ |
| | TLLMDLGQFLLFCHISSHQHDGMEAYVKVDSCPEEPQLRMKNNEEAEDYDDDLTDSEMDVV |
| | RFDDDNSPSFIQIRSVAKKHPKTWVHYIAAEEEDWDYAPLVLAPDDRSYKSQYLNNGPQRIG |
| | RKYKKVRFMAYTDETFKTREAIQHESGILGPLLYGEVGDTLLIIFKNQASRPYNIYPHGITDVR |
| | PLYSRRLPKGVKHLKDFPILPGEIFKYKWTVTVEDGPTKSDPRCLTRYYSSFVNMERDLASGL |
| | IGPLLICYKESVDQRGNQIMSDKRNVILFSVFDENRSWYLTENIQRFLPNPAGVQLEDPEFQAS |
| | NIMHSINGYVFDSLQLSVCLHEVAYWYILSIGAQTDFLSVFFSGYTFKHKMVYEDTLTLFPFS |
| | GETVFMSMENPGLWILGCHNSDFRNRGMTALLKVSSCDKNTGDYYEDSYEDISAYLLSKNN |
| | AIEPRSFSQNPPVLKRHQREITRTTLQSDQEEIDYDDTISVEMKKEDFDIYDEDENQSPRSFQKK |
| | TRHYFIAAVERLWDYGMSSSPHVLRNRAQSGSVPQFKKVVFQEFTDGSFTQPLYRGELNEHL |
| | GLLGPYIRAEVEDNIMVTFRNQASRPYSFYSSLISYEEDQRQGAEPRKNFVKPNETKTYFWKV |
| | QHHMAPTKDEFDCKAWAYFSDVDLEKDVHSGLIGPLLVCHTNTLNPAHGRQVTVQEFALFF |
| | TIFDETKSWYFTENMERNCRAPCNIQMEDPTFKENYRFHAINGYIMDTLPGLVMAQDQRIRW |
| | YLLSMGSNENIHSIHFSGHVFTVRKKEEYKMALYNLYPGVFETVEMLPSKAGIWRVECLIGEH |
| | LHAGMSTLFLVYSNKCQTPLGMASGHIRDFQITASGQYGQWAPKLARLHYSGSINAWSTKEP |
| | FSWIKVDLLAPMIIHGIKTQGARQKFSSLYISQFIIMYSLDGKKWQTYRGNSTGTLMVFFGNV |
| | DSSGIKHNIFNPPIIARYIRLHPTHYSIRSTLRMELMGCDLNSCSMPLGMESKAISDAQITASSYF |
| | TNMFATWSPSKARLHLQGRSNAWRPQVNNPKEWLQVDFQKTMKVTGVTTQGVKSLLTSM |
| | YVKEFLISSSQDGHQWTLFFQNGKVKVFQGNQDSFTPVVNSLDPPLLTRYLRIHPQSWVHQIA |
| | LRMEVLGCEAQDLYGTMTRIVGGGGSPAGSPTSTEEGTSESATPESGPGTSTEPSEGSAPGSPA |
| | GSPTSTEEGTSTEPSEGSAPGTSTEPSEGSAPGTSESATPESGPGSEPATSGSETPGSEPATSGSE |
| | TPGSPAGSPTSTEEGTSESATPESGPGTSTEPSEGSAPGTSTEPSEGSAPGSPAGSPTSTEEGTST |
| | EPSEGSAPGTSTEPSEGSAPGTSESATPESGPGTSTEPSEGSAPGTSESATPESGPGSEPATSGSE |
| | TPGTSTEPSEGSAPGTSTEPSEGSAPGTSESATPESGPGTSESATPESGPGSPAGSPTSTEEGTSES |
| | ATPESGPGSEPATSGSETPGTSESATPESGPGTSTEPSEGSAPGTSTEPSEGSAPGTSTEPSEGSA |
| | PGTSTEPSEGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTSTEPSEGSA |
| | PGTSESATPESGPGSPAGSPTSTEEGSPAGSPTSTEEGSPAGSPTSTEEGTSESATPESGPGTSTEP |
| | SEGSAPGTSESATPESGPGSEPATSGSETPGTSESATPESGPGSEPATSGSETPGTSESATPESGP |
| | GTSTEPSEGSAPGSPAGSPTSTEEGTSESATPESGPGSEPATSGSETPGTSESATPESGPGSPAGS |
| | PTSTEEGSPAGSPTSTEEGTSTEPSEGSAPGTSESATPESGPGTSESATPESGPGTSESATPESGP |
| | GSEPATSGSETPGSEPATSGSETPGSPAGSPTSTEEGTSTEPSEGSAPGTSTEPSEGSAPGSEPAT |
| | SGSETPGTSESATPESGPGTSTEPSEGSAP |
| AG144- | SGTASSSPGSSTPSGATGSPGTPGSGTASSSPGSSTPSGATGSPGSSTPSGATGSPGSSPSASTGT |
| FVIII | GPGSSPSASTGTGPGASPGTSSTGSPGTPGSGTASSSPGSSTPSGATGSPGSSPSASTGTGPGSSP |
| BDD2- | SASTGTGPGASPGATRRYYLGAVELSWDYMQSDLGELPVDARFPPRVPKSFPFNTSVVYKKT |
| FXIa- | LFVEFTVHLFNIAKPRPPWMGLLGPTIQAEVYDTVVITLKNMASHPVSLHAVGVSYWKASEG |
| AG576 | AEYDDQTSQREKEDDKVFPGGSHTYVWQVLKENGPMASDPLCLTYSYLSHVDLVKDLNSGL |
| | IGALLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFLTAQ |
| | TLLMDLGQFLLFCHISSHQHDGMEAYVKVDSCPEEPQLRMKNNEEAEDYDDDLTDSEMDVV |
| | RFDDDNSPSFIQIRSVAKKHPKTWVHYIAAEEEDWDYAPLVLAPDDRSYKSQYLNNGPQRIG |
| | RKYKKVRFMAYTDETFKTREAIQHESGILGPLLYGEVGDTLLIIFKNQASRPYNIYPHGITDVR |
| | PLYSRRLPKGVKHLKDFPILPGEIFKYKWTVTVEDGPTKSDPRCLTRYYSSFVNMERDLASGL |
| | IGPLLICYKESVDQRGNQIMSDKRNVILFSVFDENRSWYLTENIQRFLPNPAGVQLEDPEFQAS |
| | NIMHSINGYVFDSLQLSVCLHEVAYWYILSIGAQTDFLSVFFSGYTFKHKMVYEDTLTLFPFS |
| | GETVFMSMENPGLWILGCHNSDFRNRGMTALLKVSSCDKNTGDYYEDSYEDISAYLLSKNN |
| | AIEPRSFSQNPPVLKRHQREITRTTLQSDQEEIDYDDTISVEMKKEDFDIYDEDENQSPRSFQKK |
| | TRHYFIAAVERLWDYGMSSSPHVLRNRAQSGSVPQFKKVVFQEFTDGSFTQPLYRGELNEHL |
| | GLLGPYIRAEVEDNIMVTFRNQASRPYSFYSSLISYEEDQRQGAEPRKNFVKPNETKTYFWKV |
| | QHHMAPTKDEFDCKAWAYFSDVDLEKDVHSGLIGPLLVCHTNTLNPAHGRQVTVQEFALFF |
| | TIFDETKSWYFTENMERNCRAPCNIQMEDPTFKENYRFHAINGYIMDTLPGLVMAQDQRIRW |
| | YLLSMGSNENIHSIHFSGHVFTVRKKEEYKMALYNLYPGVFETVEMLPSKAGIWRVECLIGEH |
| | LHAGMSTLFLVYSNKCQTPLGMASGHIRDFQITASGQYGQWAPKLARLHYSGSINAWSTKEP |
| | FSWIKVDLLAPMIIHGIKTQGARQKFSSLYISQFIIMYSLDGKKWQTYRGNSTGTLMVFFGNV |
| | DSSGIKHNIFNPPIIARYIRLHPTHYSIRSTLRMELMGCDLNSCSMPLGMESKAISDAQITASSYF |
| | TNMFATWSPSKARLHLQGRSNAWRPQVNNPKEWLQVDFQKTMKVTGVTTQGVKSLLTSM |
| | YVKEFLISSSQDGHQWTLFFQNGKVKVFQGNQDSFTPVVNSLDPPLLTRYLRIHPQSWVHQIA |
| | LRMEVLGCEAQDLYGKLTRAETGPGTPGSGTASSSPGSSTPSGATGSPGSSPSASTGTGPGSSP |
| | SASTGTGPGSSTPSGATGSPGSSTPSGATGSPGASPGTSSTGSPGASPGTSSTGSPGASPGTSSTG |
| | SPGTPGSGTASSSPGASPGTSSTGSPGASPGTSSTGSPGASPGTSSTGSPGSSPSASTGTGPGTPG |
| | SGTASSSPGASPGTSSTGSPGASPGTSSTGSPGASPGTSSTGSPGSSTPSGATGSPGSSTPSGATG |
| | SPGASPGTSSTGSPGTPGSGTASSSPGSSTPSGATGSPGSSTPSGATGSPGSSTPSGATGSPGSSP |
| | SASTGTGPGASPGTSSTGSPGASPGTSSTGSPGTPGSGTASSSPGASPGTSSTGSPGASPGTSSTG |
| | SPGASPGTSSTGSPGASPGTSSTGSPGTPGSGTASSSPGSSTPSGATGSPGTPGSGTASSSPGSST |
| | PSGATGSPGTPGSGTASSSPGSSTPSGATGSPGSSTPSGATGSPGSSPSASTGTGPGSSPSASTGT |
| | GPGASPGTSSTGSPGTPGSGTASSSPGSSTPSGATGSPGSSPSASTGTGPGSSPSASTGTGPGASP |
| | GTSSTGS |
| AE144- | GSEPATSGSETPGTSESATPESGPGSEPATSGSETPGSPAGSPTSTEEGTSTEPSEGSAPGSEPAT |
| FXIa-FVIII | SGSETPGSEPATSGSETPGSEPATSGSETPGTSTEPSEGSAPGTSESATPESGPGSEPATSGSETP |
| BDD2- | GTSTEPSEGSAPGKLTRAETGATRRYYLGAVELSWDYMQSDLGELPVDARFPPRVPKSFPFNT |
| AE864 | SVVYKKTLFVEFTDHLFNIAKPRPPWMGLLGPTIQAEVYDTVVITLKNMASHPVSLHAVGVS |
| | YWKASEGAEYDDQTSQREKEDDKVFPGGSHTYVWQVLKENGPMASDPLCLTYSYLSHVDL |
| | VKDLNSGLIGALLVCREGSLAKEKTQTLHKFILLFAVFDEGKSWHSETKNSLMQDRDAASAR |
| | AWPKMHTVNGYVNRSLPGLIGCHRKSVYWHVIGMGTTPEVHSIFLEGHTFLVRNHRQASLEI |
| | SPITFLTAQTLLMDLGQFLLFCHISSHQHDGMEAYVKVDSCPEEPQLRMKNNEEAEDYDDDL |
| | TDSEMDVVRFDDDNSPSFIQIRSVAKKHPKTWVHYIAAEEEDWDYAPLVLAPDDRSYKSQYL |
| | NNGPQRIGRKYKKVRFMAYTDETFKTREAIQHESGILGPLLYGEVGDTLLIIFKNQASRPYNIY |
| | PHGITDVRPLYSRRLPKGVKHLKDFPILPGEIFKYKWTVTVEDGPTKSDPRCLTRYYSSFVNM |
| | ERDLASGLIGPLLICYKESVDQRGNQIMSDKRNVILFSVFDENRSWYLTENIQRFLPNPAGVQL |
| | EDPEFQASNIMHSINGYVFDSLQLSVCLHEVAYWYILSIGAQTDFLSVFFSGYTFKHKMVYED |
| | TLTLFPFSGETVFMSMENPGLWILGCHNSDFRNRGMTALLKVSSCDKNTGDYYEDSYEDISA |
| | YLLSKNNAIEPRSFSQNPPVLKRHQREITRTTLQSDQEEIDYDDTISVEMKKEDFDIYDEDENQ |
| | SPRSFQKKTRHYFIAAVERLWDYGMSSSPHVLRNRAQSGSVPQFKKVVFQEFTDGSFTQPLY |
| | RGELNEHLGLLGPYIRAEVEDNIMVTFRNQASRPYSFYSSLISYEEDQRQGAEPRKNFVKPNE |
| | TKTYFWKVQHHMAPTKDEFDCKAWAYFSDVDLEKDVHSGLIGPLLVCHTNTLNPAHGRQV |
| | TVQEFALFFTIFDETKSWYFTENMERNCRAPCNIQMEDPTFKENYRFHAINGYIMDTLPGLVM |
| | AQDQRIRWYLLSMGSNENIHSIHFSGHVFTVRKKEEYKMALYNLYPGVFETVEMLPSKAGIW |
| | RVECLIGEHLHAGMSTLFLVYSNKCQTPLGMASGHIRDFQITASGQYGQWAPKLARLHYSGS |
| | INAWSTKEPFSWIKVDLLAPMIIHGIKTQGARQKFSSLYISQFIIMYSLDGKKWQTYRGNSTGT |
| | LMVFFGNVDSSGIKHNIFNPPIIARYIRLHPTHYSIRSTLRMELMGCDLNSCSMPLGMESKAISD |
| | AQITASSYFTNMFATWSPSKARLHLQGRSNAWRPQVNNPKEWLQVDFQKTMKVTGVTTQG |
| | VKSLLTSMYVKEFLISSSQDGHQWTLFFQNGKVKVFQGNQDSFTPVVNSLDPPLLTRYLRIHP |
| | QSWVHQIALRMEVLGCEAQDLYGGSPAGSPTSTEEGTSESATPESGPGTSTEPSEGSAPGSPA |
| | GSPTSTEEGTSTEPSEGSAPGTSTEPSEGSAPGTSESATPESGPGSEPATSGSETPGSEPATSGSE |
| | TPGSPAGSPTSTEEGTSESATPESGPGTSTEPSEGSAPGTSTEPSEGSAPGSPAGSPTSTEEGTST |
| | EPSEGSAPGTSTEPSEGSAPGTSESATPESGPGTSTEPSEGSAPGTSESATPESGPGSEPATSGSE |
| | TPGTSTEPSEGSAPGTSTEPSEGSAPGTSESATPESGPGTSESATPESGPGSPAGSPTSTEEGTSES |
| | ATPESGPGSEPATSGSETPGTSESATPESGPGTSTEPSEGSAPGTSTEPSEGSAPGTSTEPSEGSA |
| | PGTSTEPSEGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTSTEPSEGSA |
| | PGTSESATPESGPGSPAGSPTSTEEGSPAGSPTSTEEGSPAGSPTSTEEGTSESATPESGPGTSTEP |
| | SEGSAPGTSESATPESGPGSEPATSGSETPGTSESATPESGPGSEPATSGSETPGTSESATPESGP |
| | GTSTEPSEGSAPGSPAGSPTSTEEGTSESATPESGPGSEPATSGSETPGTSESATPESGPGSPAGS |
| | PTSTEEGSPAGSPTSTEEGTSTEPSEGSAPGTSESATPESGPGTSESATPESGPGTSESATPESGP |
| | GSEPATSGSETPGSEPATSGSETPGSPAGSPTSTEEGTSTEPSEGSAPGTSTEPSEGSAPGSEPAT |
| | SGSETPGTSESATPESGPGTSTEPSEGSAP |
| AE144- | GSEPATSGSETPGTSESATPESGPGSEPATSGSETPGSPAGSPTSTEEGTSTEPSEGSAPGSEPAT |
| FVIII | SGSETPGSEPATSGSETPGSEPATSGSETPGTSTEPSEGSAPGTSESATPESGPGSEPATSGSETP |
| BDD2- | GTSTEPSEGSAPGATRRYYLGAVELSWDYMQSDLGELPVDARFPPRVPKSFPFNTSVVYKKT |
| Y2332- | LFVEFTVHLFNIAKPRPPWMGLLGPTIQAEVYDTVVITLKNMASHPVSLHAVGVSYWKASEG |
| Thrombin- | AEYDDQTSQREKEDDKVFPGGSHTYVWQVLKENGPMASDPLCLTYSYLSHVDLVKDLNSGL |
| AE864 | IGALLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFLTAQ |
| | TLLMDLGQFLLFCHISSHQHDGMEAYVKVDSCPEEPQLRMKNNEEAEDYDDDLTDSEMDVV |
| | RFDDDNSPSFIQIRSVAKKHPKTWVHYIAAEEEDWDYAPLVLAPDDRSYKSQYLNNGPQRIG |
| | RKYKKVRFMAYTDETFKTREAIQHESGILGPLLYGEVGDTLLIIFKNQASRPYNIYPHGITDVR |
| | PLYSRRLPKGVKHLKDFPILPGEIFKYKWTVTVEDGPTKSDPRCLTRYYSSFVNMERDLASGL |
| | IGPLLICYKESVDQRGNQIMSDKRNVILFSVFDENRSWYLTENIQRFLPNPAGVQLEDPEFQAS |
| | NIMHSINGYVFDSLQLSVCLHEVAYWYILSIGAQTDFLSVFFSGYTFKHKMVYEDTLTLFPFS |
| | GETVFMSMENPGLWILGCHNSDFRNRGMTALLKVSSCDKNTGDYYEDSYEDISAYLLSKNN |
| | AIEPRSFSQNPPVLKRHQREITRTTLQSDQEEIDYDDTISVEMKKEDFDIYDEDENQSPRSFQKK |
| | TRHYFIAAVERLWDYGMSSSPHVLRNRAQSGSVPQFKKVVFQEFTDGSFTQPLYRGELNEHL |
| | GLLGPYIRAEVEDNIMVTFRNQASRPYSFYSSLISYEEDQRQGAEPRKNFVKPNETKTYFWKV |
| | QHHMAPTKDEFDCKAWAYFSDVDLEKDVHSGLIGPLLVCHTNTLNPAHGRQVTVQEFALFF |
| | TIFDETKSWYFTENMERNCRAPCNIQMEDPTFKENYRFHAINGYIMDTLPGLVMAQDQRIRW |
| | YLLSMGSNENIHSIHFSGHVFTVRKKEEYKMALYNLYPGVFETVEMLPSKAGIWRVECLIGEH |
| | LHAGMSTLFLVYSNKCQTPLGMASGHIRDFQITASGQYGQWAPKLARLHYSGSINAWSTKEP |
| | FSWIKVDLLAPMIIHGIKTQGARQKFSSLYISQFIIMYSLDGKKWQTYRGNSTGTLMVFFGNV |
| | DSSGIKHNIFNPPIIARYIRLHPTHYSIRSTLRMELMGCDLNSCSMPLGMESKAISDAQITASSYF |
| | TNMFATWSPSKARLHLQGRSNAWRPQVNNPKEWLQVDFQKTMKVTGVTTQGVKSLLTSM |
| | YVKEFLISSSQDGHQWTLFFQNGKVKVFQGNQDSFTPVVNSLDPPLLTRYLRIHPQSWVHQIA |
| | LRMEVLGCEAQDLYGLTPRSLLVGGSPAGSPTSTEEGTSESATPESGPGTSTEPSEGSAPGSPA |
| | GSPTSTEEGTSTEPSEGSAPGTSTEPSEGSAPGTSESATPESGPGSEPATSGSETPGSEPATSGSE |
| | TPGSPAGSPTSTEEGTSESATPESGPGTSTEPSEGSAPGTSTEPSEGSAPGSPAGSPTSTEEGTST |
| | EPSEGSAPGTSTEPSEGSAPGTSESATPESGPGTSTEPSEGSAPGTSESATPESGPGSEPATSGSE |
| | TPGTSTEPSEGSAPGTSTEPSEGSAPGTSESATPESGPGTSESATPESGPGSPAGSPTSTEEGTSES |
| | ATPESGPGSEPATSGSETPGTSESATPESGPGTSTEPSEGSAPGTSTEPSEGSAPGTSTEPSEGSA |
| | PGTSTEPSEGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTSTEPSEGSA |
| | PGTSESATPESGPGSPAGSPTSTEEGSPAGSPTSTEEGSPAGSPTSTEEGTSESATPESGPGTSTEP |
| | SEGSAPGTSESATPESGPGSEPATSGSETPGTSESATPESGPGSEPATSGSETPGTSESATPESGP |
| | GTSTEPSEGSAPGSPAGSPTSTEEGTSESATPESGPGSEPATSGSETPGTSESATPESGPGSPAGS |
| | PTSTEEGSPAGSPTSTEEGTSTEPSEGSAPGTSESATPESGPGTSESATPESGPGTSESATPESGP |
| | GSEPATSGSETPGSEPATSGSETPGSPAGSPTSTEEGTSTEPSEGSAPGTSTEPSEGSAPGSEPAT |
| | SGSETPGTSESATPESGPGTSTEPSEGSAP |
| AE864- | GSPAGSPTSTEEGTSESATPESGPGTSTEPSEGSAPGSPAGSPTSTEEGTSTEPSEGSAPGTSTEPS |
| FVIII- | EGSAPGTSESATPESGPGSEPATSGSETPGSEPATSGSETPGSPAGSPTSTEEGTSESATPESGPG |
| MMP-17- | TSTEPSEGSAPGTSTEPSEGSAPGSPAGSPTSTEEGTSTEPSEGSAPGTSTEPSEGSAPGTSESATP |
| AE144 | ESGPGTSTEPSEGSAPGTSESATPESGPGSEPATSGSETPGTSTEPSEGSAPGTSTEPSEGSAPGT |
| | SESATPESGPGTSESATPESGPGSPAGSPTSTEEGTSESATPESGPGSEPATSGSETPGTSESATPE |
| | SGPGTSTEPSEGSAPGTSTEPSEGSAPGTSTEPSEGSAPGTSTEPSEGSAPGTSTEPSEGSAPGTS |
| | TEPSEGSAPGSPAGSPTSTEEGTSTEPSEGSAPGTSESATPESGPGSEPATSGSETPGTSESATPES |
| | GPGSEPATSGSETPGTSESATPESGPGTSTEPSEGSAPGTSESATPESGPGSPAGSPTSTEEGSPA |
| | GSPTSTEEGSPAGSPTSTEEGTSESATPESGPGTSTEPSEGSAPGTSESATPESGPGSEPATSGSE |
| | TPGTSESATPESGPGSEPATSGSETPGTSESATPESGPGTSTEPSEGSAPGSPAGSPTSTEEGTSES |
| | ATPESGPGSEPATSGSETPGTSESATPESGPGSPAGSPTSTEEGSPAGSPTSTEEGTSTEPSEGSA |
| | PGTSESATPESGPGTSESATPESGPGTSESATPESGPGSEPATSGSETPGSEPATSGSETPGSPAG |
| | SPTSTEEGTSTEPSEGSAPGTSTEPSEGSAPGSEPATSGSETPGTSESATPESGPGTSTEPSEGSAP |
| | GATRRYYLGAVELSWDYMQSDLGELPVDARFPPRVPKSFPFNTSVVYKKTLFVEFTDHLFNI |
| | AKPRPPWMGLLGPTIQAEVYDTVVITLKNMASHPVSLHAVGVSYWKASEGAEYDDQTSQRE |
| | KEDDKVFPGGSHTYVWQVLKENGPMASDPLCLTYSYLSHVDLVKDLNSGLIGALLVCREGS |
| | LAKEKTQTLHKFILLFAVFDEGKSWHSETKNSLMQDRDAASARAWPKMHTVNGYVNRSLPG |
| | LIGCHRKSVYWHVIGMGTTPEVHSIFLEGHTFLVRNHRQASLEISPITFLTAQTLLMDLGQFLL |
| | FCHISSHQHDGMEAYVKVDSCPEEPQLRMKNNEEAEDYDDDLTDSEMDVVRFDDDNSPSFIQ |
| | IRSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNSRHP |
| | STRQKQFNATTIPENDIEKTDPWFAHRTPMPKIQNVSSSDLLMLLRQSPTPHGLSLSDLQEAKY |
| | ETFSDDPSPGAIDSNNSLSEMTHFRPQLHHSGDMVFTPESGLQLRLNEKLGTTAATELKKLDF |
| | KVSSTSNNLISTIPSDNLAAGTDNTSSLGPPSMPVHYDSQLDTTLFGKKSSPLTESGGPLSLSEE |
| | NNDSKLLESGLMNSQESSWGKNVSSTESGRLFKGKRAHGPALLTKDNALFKVSISLLKTNKT |
| | SNNSATNRKTHIDGPSLLIENSPSVWQNILESDTEFKKVTPLIHDRMLMDKNATALRLNHMSN |
| | KTTSSKNMEMVQQKKEGPIPPDAQNPDMSFFKMLFLPESARWIQRTHGKNSLNSGQGPSPKQ |
| | LVSLGPEKSVEGQNFLSEKNKVVVGKGEFTKDVGLKEMVFPSSRNLFLTNLDNLHENNTHNQ |
| | EKKIQEEIEKKETLIQENVVLPQIHTVTGTKNFMKNLFLLSTRQNVEGSYDGAYAPVLQDFRS |
| | LNDSTNRTKKHTAHFSKKGEEENLEGLGNQTKQIVEKYACTTRISPNTSQQNFVTQRSKRAL |
| | KQFRLPLEETELEKRIIVDDTSTQWSKNMKHLTPSTLTQIDYNEKEKGAITQSPLSDCLTRSHSI |
| | PQANRSPLPIAKVSSFPSIRPIYLTRVLFQDNSSHLPAASYRKKDSGVQESSHFLQGAKKNNLS |
| | LAILTLEMTGDQREVGSLGTSATNSVTYKKVENTVLPKPDLPKTSGKVELLPKVHIYQKDLFP |
| | TETSNGSPGHLDLVEGSLLQGTEGAIKWNEANRPGKVPFLRVATESSAKTPSKLLDPLAWDN |
| | HYGTQIPKEEWKSQEKSPEKTAFKKKDTILSLNACESNHAIAAINEGQNKPEIEVTWAKQGRT |
| | ERLCSQNPPVLKRHQREITRTTLQSDQEEIDYDDTISVEMKKEDFDIYDEDENQSPRSFQKKTR |
| | HYFIAAVERLWDYGMSSSPHVLRNRAQSGSVPQFKKVVFQEFTDGSFTQPLYRGELNEHLGL |
| | LGPYIRAEVEDNIMVTFRNQASRPYSFYSSLISYEEDQRQGAEPRKNFVKPNETKTYFWKVQH |
| | HMAPTKDEFDCKAWAYFSDVDLEKDVHSGLIGPLLVCHTNTLNPAHGRQVTVQEFALFFTIF |
| | DETKSWYFTENMERNCRAPCNIQMEDPTFKENYRFHAINGYIMDTLPGLVMAQDQRIRWYL |
| | LSMGSNENIHSIHFSGHVFTVRKKEEYKMALYNLYPGVFETVEMLPSKAGIWRVECLIGEHLH |
| | AGMSTLFLVYSNKCQTPLGMASGHIRDFQITASGQYGQWAPKLARLHYSGSINAWSTKEPFS |
| | WIKVDLLAPMIIHGIKTQGARQKFSSLYISQFIIMYSLDGKKWQTYRGNSTGTLMVFFGNVDS |
| | SGIKHNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTSMYV |
| | KEFLISSSQDGHQWTLFFQNGKVKVFQGNQDSFTPVVNSLDPPLLTRYLRIHPQSWVHQIALR |
| | MEVLGCEAQDLYGAPLGLRLRGGSEPATSGSETPGTSESATPESGPGSEPATSGSETPGSPAGS |
| | PTSTEEGTSTEPSEGSAPGSEPATSGSETPGSEPATSGSETPGSEPATSGSETPGTSTEPSEGSAP |
| | GTSESATPESGPGSEPATSGSETPGTSTEPSEGSAP |
| AF144- | GTSTPESGSASPGTSPSGESSTAPGTSPSGESSTAPGSTSSTAESPGPGSTSESPSGTAPGSTSSTA |
| FXIIa- | ESPGPGTSPSGESSTAPGTSTPESGSASPGSTSSTAESPGPGTSPSGESSTAPGTSPSGESSTAPGT |
| FVIII- | SPSGESSTAPGTMTRIVGGATRRYYLGAVELSWDYMQSDLGELPVDARFPPRVPKSFPFNTSV |
| FXIIa- | VYKKTLFVEFTDHLFNIAKPRPPWMGLLGPTIQAEVYDTVVITLKNMASHPVSLHAVGVSYW |
| AF864 | KASEGAEYDDQTSQREKEDDKVFPGGSHTYVWQVLKENGPMASDPLCLTYSYLSHVDLVK |
| | DLNSGLIGALLVCREGSLAKEKTQTLHKFILLFAVFDEGKSWHSETKNSLMQDRDAASARAW |
| | PKMHTVNGYVNRSLPGLIGCHRKSVYWHVIGMGTTPEVHSIFLEGHTFLVRNHRQASLEISPI |
| | TFLTAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQYLNN |
| | GPQRIGRKYKKVRFMAYTDETFKTREAIQHESGILGPLLYGEVGDTLLIIFKNQASRPYNIYPH |
| | GITDVRPLYSRRLPKGVKHLKDFPILPGEIFKYKWTVTVEDGPTKSDPRCLTRYYSSFVNMER |
| | DLASGLIGPLLICYKESVDQRGNQIMSDKRNVILFSVFDENRSWYLTENIQRFLPNPAGVQLED |
| | PEFQASNIMHSINGYVFDSLQLSVCLHEVAYWYILSIGAQTDFLSVFFSGYTFKHKMVYEDTL |
| | TLFPFSGETVFMSMENPG/LWILGCHNSDFRNRGMTALLKVSSCDKNTGDYYEDSYEDISAYL |
| | LSKNNAIEPRSFSQNSRHPSTRQKQFNATTIPENDIEKTDPWFAHRTPMPKIQNVSSSDLLMLL |
| | RQSPTPHGLSLSDLQEAKYETFSDDPSPGAIDSNNSLSEMTHFRPQLHHSGDMVFTPESGLQLR |
| | LNEKLGTTAATELKKLDFKVSSTSNNLISTIPSDNLAAGTDNTSSLGPPSMPVHYDSQLDTTLF |
| | GKKSSPLTESGGPLSLSEENNDSKLLESGLMNSQESSWGKNVSSTESGRLFKGKRAHGPALLT |
| | KDNALFKVSISLLKTNKTSNNSATNRKTHIDGPSLLIENSPSVWQNILESDTEFKKVTPLIHDR |
| | MLMDKNATALRLNHMSNKTTSSKNMEMVQQKKEGPIPPDAQNPDMSFFKMLFLPESARWIQ |
| | RTHGKNSLNSGQGPSPKQLVSLGPEKSVEGQNFLSEKNKVVVGKGEFTKDVGLKEMVFPSSR |
| | NLFLTNLDNLHENNTHNQEKKIQEEIEKKETLIQENVVLPQIHTVTGTKNFMKNLFLLSTRQN |
| | VEGSYDGAYAPVLQDFRSLNDSTNRTKKHTAHFSKKGEEENLEGLGNQTKQIVEKYACTTRI |
| | SPNTSQQNFVTQRSKRALKQFRLPLEETELEKRIIVDDTSTQWSKNMKHLTPSTLTQIDYNEKE |
| | KGAITQSPLSDCLTRSHSIPQANRSPLPIAKVSSFPSIRPIYLTRVLFQDNSSHLPAASYRKKDSG |
| | VQESSHFLQGAKKNNLSLAILTLEMTGDQREVGSLGTSATNSVTYKKVENTVLPKPDLPKTS |
| | GKVELLPKVHIYQKDLFPTETSNGSPGHLDLVEGSLLQGTEGAIKWNEANRPGKVPFLRVATE |
| | SSAKTPSKLLDPLAWDNHYGTQIPKEEWKSQEKSPEKTAFKKKDTILSLNACESNHAIAAINE |
| | GQNKPEIEVTWAKQGRTERLCSQNPPVLKRHQREITRTTLQSDQEEIDYDDTISVEMKKEDFD |
| | IYDEDENQSPRSFQKKTRHYFIAAVERLWDYGMSSSPHVLRNRAQSGSVPQFKKVVFQEFTD |
| | GSFTQPLYRGELNEHLGLLGPYIRAEVEDNIMVTFRNQASRPYSFYSSLISYEEDQRQGAEPRK |
| | NFVKPNETKTYFWKVQHHMAPTKDEFDCKAWAYFSDVDLEKDVHSGLIGPLLVCHTNTLNP |
| | AHGRQVTVQEFALFFTIFDETKSWYFTENMERNCRAPCNIQMEDPTFKENYRFHAINGYIMD |
| | TLPGLVMAQDQRIRWYLLSMGSNENIHSIHFSGHVFTVRKKEEYKMALYNLYPGVFETVEML |
| | PSKAGIWRVECLIGEHLHAGMSTLFLVYSNKCQTPLGMASGHIRDFQITASGQYGQWAPKLA |
| | RLHYSGSINAWSTKEPFSWIKVDLLAPMIIHGIKTQGARQKFSSLYISQFIIMYSLDGKKWQTY |
| | RGNSTGTLMVFFGNVDSSGIKHNIFNPPIIARYIRLHPTHYSIRSTLRMELMGCDLNSCSMPLG |
| | MESKAISDAQITASSYFTNMFATWSPSKARLHLQGRSNAWRPQVNNPKEWLQVDFQKTMKV |
| | TGVTTQGVKSLLTSMYVKEFLISSSQDGHQWTLFFQNGKVKVFQGNQDSFTPVVNSLDPPLL |
| | TRYLRIHPQSWVHQIALRMEVLGCEAQDLYGTMTRIVGGGSTSESPSGTAPGTSPSGESSTAP |
| | GSTSESPSGTAPGSTSESPSGTAPGTSTPESGSASPGTSTPESGSASPGSTSESPSGTAPGSTSESP |
| | SGTAPGTSPSGESSTAPGSTSESPSGTAPGTSPSGESSTAPGTSPSGESSTAPGSTSSTAESPGPGT |
| | SPSGESSTAPGTSPSGESSTAPGSTSSTAESPGPGTSTPESGSASPGTSTPESGSASPGSTSESPSG |
| | TAPGSTSESPSGTAPGTSTPESGSASPGSTSSTAESPGPGTSTPESGSASPGSTSESPSGTAPGTSP |
| | SGESSTAPGSTSSTAESPGPGTSPSGESSTAPGTSTPESGSASPGSTSSTAESPGPGSTSSTAESPG |
| | PGSTSSTAESPGPGSTSSTAESPGPGTSPSGESSTAPGSTSESPSGTAPGSTSESPSGTAPGTSTPE |
| | SGPXXXGASASGAPSTXXXXSESPSGTAPGSTSESPSGTAPGSTSESPSGTAPGSTSESPSGTAP |
| | GSTSESPSGTAPGSTSESPSGTAPGTSTPESGSASPGTSPSGESSTAPGTSPSGESSTAPGSTSSTA |
| | ESPGPGTSPSGESSTAPGTSTPESGSASPGSTSESPSGTAPGSTSESPSGTAPGTSPSGESSTAPGS |
| | TSESPSGTAPGTSTPESGSASPGTSTPESGSASPGSTSESPSGTAPGTSTPESGSASPGSTSSTAES |
| | PGPGSTSESPSGTAPGSTSESPSGTAPGTSPSGESSTAPGSTSSTAESPGPGTSPSGESSTAPGTST |
| | PESGSASPGTSPSGESSTAPGTSPSGESSTAPGTSPSGESSTAPGSTSSTAESPGPGSTSSTAESPG |
| | PGTSPSGESSTAPGSSPSASTGTGPGSSTPSGATGSPGSSTPSGATGSP |
| AE864- | GSPAGSPTSTEEGTSESATPESGPGTSTEPSEGSAPGSPAGSPTSTEEGTSTEPSEGSAPGTSTEPS |
| FVIII- | EGSAPGTSESATPESGPGSEPATSGSETPGSEPATSGSETPGSPAGSPTSTEEGTSESATPESGPG |
| FXIa- | TSTEPSEGSAPGTSTEPSEGSAPGSPAGSPTSTEEGTSTEPSEGSAPGTSTEPSEGSAPGTSESATP |
| AE144 | ESGPGTSTEPSEGSAPGTSESATPESGPGSEPATSGSETPGTSTEPSEGSAPGTSTEPSEGSAPGT |
| | SESATPESGPGTSESATPESGPGSPAGSPTSTEEGTSESATPESGPGSEPATSGSETPGTSESATPE |
| | SGPGTSTEPSEGSAPGTSTEPSEGSAPGTSTEPSEGSAPGTSTEPSEGSAPGTSTEPSEGSAPGTS |
| | TEPSEGSAPGSPAGSPTSTEEGTSTEPSEGSAPGTSESATPESGPGSEPATSGSETPGTSESATPES |
| | GPGSEPATSGSETPGTSESATPESGPGTSTEPSEGSAPGTSESATPESGPGSPAGSPTSTEEGSPA |
| | GSPTSTEEGSPAGSPTSTEEGTSESATPESGPGTSTEPSEGSAPGTSESATPESGPGSEPATSGSE |
| | TPGTSESATPESGPGSEPATSGSETPGTSESATPESGPGTSTEPSEGSAPGSPAGSPTSTEEGTSES |
| | ATPESGPGSEPATSGSETPGTSESATPESGPGSPAGSPTSTEEGSPAGSPTSTEEGTSTEPSEGSA |
| | PGTSESATPESGPGTSESATPESGPGTSESATPESGPGSEPATSGSETPGSEPATSGSETPGSPAG |
| | SPTSTEEGTSTEPSEGSAPGTSTEPSEGSAPGSEPATSGSETPGTSESATPESGPGTSTEPSEGSAP |
| | GATRRYYLGAVELSWDYMQSDLGELPVDARFPPRVPKSFPFNTSVVYKKTLFVEFTDHLFNI |
| | AKPRPPWMGLLGPTIQAEVYDTVVITLKNMASHPVSLHAVGVSYWKASEGAEYDDQTSQRE |
| | KEDDKVFPGGSHTYVWQVLKENGPMASDPLCLTYSYLSHVDLVKDLNSGLIGALLVCREGS |
| | LAKEKTQTLHKFILLFAVFDEGKSWHSETKNSLMQDRDAASARAWPKMHTVNGYVNRSLPG |
| | LIGCHRKSVYWHVIGMGTTPEVHSIFLEGHTFLVRNHRQASLEISPITFLTAQTLLMDLGQFLL |
| | FCHISSHQHDGMEAYVKVDSCPEEPQLRMKNNEEAEDYDDDLTDSEMDVVRFDDDNSPSFIQ |
| | IRSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNSRHP |
| | STRQKQFNATTIPENDIEKTDPWFAHRTPMPKIQNVSSSDLLMLLRQSPTPHGLSLSDLQEAKY |
| | ETFSDDPSPGAIDSNNSLSEMTHFRPQLHHSGDMVFTPESGLQLRLNEKLGTTAATELKKLDF |
| | KVSSTSNNLISTIPSDNLAAGTDNTSSLGPPSMPVHYDSQLDTTLFGKKSSPLTESGGPLSLSEE |
| | NNDSKLLESGLMNSQESSWGKNVSSTESGRLFKGKRAHGPALLTKDNALFKVSISLLKTNKT |
| | SNNSATNRKTHIDGPSLLIENSPSVWQNILESDTEFKKVTPLIHDRMLMDKNATALRLNHMSN |
| | KTTSSKNMEMVQQKKEGPIPPDAQNPDMSFFKMLFLPESARWIQRTHGKNSLNSGQGPSPKQ |
| | LVSLGPEKSVEGQNFLSEKNKVVVGKGEFTKDVGLKEMVFPSSRNLFLTNLDNLHENNTHNQ |
| | EKKIQEEIEKKETLIQENVVLPQIHTVTGTKNFMKNLFLLSTRQNVEGSYDGAYAPVLQDFRS |
| | LNDSTNRTKKHTAHFSKKGEEENLEGLGNQTKQIVEKYACTTRISPNTSQQNFVTQRSKRAL |
| | KQFRLPLEETELEKRIIVDDTSTQWSKNMKHLTPSTLTQIDYNEKEKGAITQSPLSDCLTRSHSI |
| | PQANRSPLPIAKVSSFPSIRPIYLTRVLFQDNSSHLPAASYRKKDSGVQESSHFLQGAKKNNLS |
| | LAILTLEMTGDQREVGSLGTSATNSVTYKKVENTVLPKPDLPKTSGKVELLPKVHIYQKDLFP |
| | TETSNGSPGHLDLVEGSLLQGTEGAIKWNEANRPGKVPFLRVATESSAKTPSKLLDPLAWDN |
| | HYGTQIPKEEWKSQEKSPEKTAFKKKDTILSLNACESNHAIAAINEGQNKPEIEVTWAKQGRT |
| | ERLCSQNPPVLKRHQREITRTTLQSDQEEIDYDDTISVEMKKEDFDIYDEDENQSPRSFQKKTR |
| | HYFIAAVERLWDYGMSSSPHVLRNRAQSGSVPQFKKVVFQEFTDGSFTQPLYRGELNEHLGL |
| | LGPYIRAEVEDNIMVTFRNQASRPYSFYSSLISYEEDQRQGAEPRKNFVKPNETKTYFWKVQH |
| | HMAPTKDEFDCKAWAYFSDVDLEKDVHSGLIGPLLVCHTNTLNPAHGRQVTVQEFALFFTIF |
| | DETKSWYFTENMERNCRAPCNIQMEDPTFKENYRFHAINGYIMDTLPGLVMAQDQRIRWYL |
| | LSMGSNENIHSIHFSGHVFTVRKKEEYKMALYNLYPGVFETVEMLPSKAGIWRVECLIGEHLH |
| | AGMSTLFLVYSNKCQTPLGMASGHIRDFQITASGQYGQWAPKLARLHYSGSINAWSTKEPFS |
| | WIKVDLLAPMIIHGIKTQGARQKFSSLYISQFIIMYSLDGKKWQTYRGNSTGTLMVFFGNVDS |
| | SGIKHNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTSMYV |
| | KEFLISSSQDGHQWTLFFQNGKVKVFQGNQDSFTPVVNSLDPPLLTRYLRIHPQSWVHQIALR |
| | MEVLGCEAQDLYGKLTRAETGGSEPATSGSETPGTSESATPESGPGSEPATSGSETPGSPAGSP |
| | TSTEEGTSTEPSEGSAPGSEPATSGSETPGSEPATSGSETPGSEPATSGSETPGTSTEPSEGSAPG |
| | TSESATPESGPGSEPATSGSETPGTSTEPSEGSAP |
| AE144- | GSEPATSGSETPGTSESATPESGPGSEPATSGSETPGSPAGSPTSTEEGTSTEPSEGSAPGSEPAT |
| FXIa-FVIII | SGSETPGSEPATSGSETPGSEPATSGSETPGTSTEPSEGSAPGTSESATPESGPGSEPATSGSETP |
| BDD9- | GTSTEPSEGSAPGKLTRAETGATRRYYLGAVELSWDYMQSDLGELPVDARFPPRVPKSFPFNT |
| AE864 | SVVYKKTLFVEFTDHLFNIAKPRPPWMGLLGPTIQAEVYDTVVITLKNMASHPVSLHAVGVS |
| | YWKASEGAEYDDQTSQREKEDDKVFPGGSHTYVWQVLKENGPMASDPLCLTYSYLSHVDL |
| | VKDLNSGLIGALLVCREGSLAKEKTQTLHKFILLFAVFDEGKSWHSETKNSLMQDRDAASAR |
| | AWPKMHTVNGYVNRSLPGLIGCHRKSVYWHVIGMGTTPEVHSIFLEGHTFLVRNHRQASLEI |
| | SPITFLTAQTLLMDLGQFLLFCHISSHQHDGMEAYVKVDSCPEEPQLRMKNNEEAEDYDDDL |
| | TDSEMDVVRFDDDNSPSFIQIRSVAKKHPKTWVHYIAAEEEDWDYAPLVLAPDDRSYKSQYL |
| | NNGPQRIGRKYKKVRFMAYTDETFKTREAIQHESGILGPLLYGEVGDTLLIIFKNQASRPYNIY |
| | PHGITDVRPLYSRRLPKGVKHLKDFPILPGEIFKYKWTVTVEDGPTKSDPRCLTRYYSSFVNM |
| | ERDLASGLIGPLLICYKESVDQRGNQIMSDKRNVILFSVFDENRSWYLTENIQRFLPNPAGVQL |
| | EDPEFQASNIMHSINGYVFDSLQLSVCLHEVAYWYILSIGAQTDFLSVFFSGYTFKHKMVYED |
| | TLTLFPFSGETVFMSMENPGLWILGCHNSDFRNRGMTALLKVSSCDKNTGDYYEDSYEDISA |
| | YLLSKNNAIEPRSFSQNPPVLKRHQREITRTTLQSDQEEIDYDDTISVEMKKEDFDIYDEDENQ |
| | SPRSFQKKTRHYFIAAVERLWDYGMSSSPHVLRNRAQSGSVPQFKKVVFQEFTDGSFTQPLY |
| | RGELNEHLGLLGPYIRAEVEDNIMVTFRNQASRPYSFYSSLISYEEDQRQGAEPRKNFVKPNE |
| | TKTYFWKVQHHMAPTKDEFDCKAWAYFSDVDLEKDVHSGLIGPLLVCHTNTLNPAHGRQV |
| | TVQEFALFFTIFDETKSWYFTENMERNCRAPCNIQMEDPTFKENYRFHAINGYIMDTLPGLVM |
| | AQDQRIRWYLLSMGSNENIHSIHFSGHVFTVRKKEEYKMALYNLYPGVFETVEMLPSKAGIW |
| | RVECLIGEHLHAGMSTLFLVYSNKCQTPLGMASGHIRDFQITASGQYGQWAPKLARLHYSGS |
| | INAWSTKEPFSWIKVDLLAPMIIHGIKTQGARQKFSSLYISQFIIMYSLDGKKWQTYRGNSTGT |
| | LMVFFGNVDSSGIKHNIFNPPIIARYIRLHPTHYSIRSTLRMELMGCDLNSCSMPLGMESKAISD |
| | AQITASSYFTNMFATWSPSKARLHLQGRSNAWRPQVNNPKEWLQVDFQKTMKVTGVTTQG |
| | VKSLLTSMYVKEFLISSSQDGHQWTLFFQNGKVKVFQGNQDSFTPVVNSLDPPLLTRYLRIHP |
| | QSWVHQIALRMEVLGCEAQDLYGGSPAGSPTSTEEGTSESATPESGPGTSTEPSEGSAPGSPA |
| | GSPTSTEEGTSTEPSEGSAPGTSTEPSEGSAPGTSESATPESGPGSEPATSGSETPGSEPATSGSE |
| | TPGSPAGSPTSTEEGTSESATPESGPGTSTEPSEGSAPGTSTEPSEGSAPGSPAGSPTSTEEGTST |
| | EPSEGSAPGTSTEPSEGSAPGTSESATPESGPGTSTEPSEGSAPGTSESATPESGPGSEPATSGSE |
| | TPGTSTEPSEGSAPGTSTEPSEGSAPGTSESATPESGPGTSESATPESGPGSPAGSPTSTEEGTSES |
| | ATPESGPGSEPATSGSETPGTSESATPESGPGTSTEPSEGSAPGTSTEPSEGSAPGTSTEPSEGSA |
| | PGTSTEPSEGSAPGTSTEPSEGSAPGTSTEPSEGSAPGSPAGSPTSTEEGTSTEPSEGSAPGTSES |
| | ATPESGPGSEPATSGSETPGTSESATPESGPGSEPATSGSETPGTSESATPESGPGTSTEPSEGSA |
| | PGTSESATPESGPGSPAGSPTSTEEGSPAGSPTSTEEGSPAGSPTSTEEGTSESATPESGPGTSTEP |
| | SEGSAPGTSESATPESGPGSEPATSGSETPGTSESATPESGPGSEPATSGSETPGTSESATPESGP |
| | GTSTEPSEGSAPGSPAGSPTSTEEGTSESATPESGPGSEPATSGSETPGTSESATPESGPGSPAGS |
| | PTSTEEGSPAGSPTSTEEGTSTEPSEGSAPGTSESATPESGPGTSESATPESGPGTSESATPESGP |
| | GSEPATSGSETPGSEPATSGSETPGSPAGSPTSTEEGTSTEPSEGSAPGTSTEPSEGSAPGSEPAT |
| | SGSETPGTSESATPESGPGTSTEPSEGSAP |
| AE48- | MAEPAGSPTSTEEGTPGSGTASSSPGSSTPSGATGSPGASPGTSSTGSPGKLTRAETGATRRYY |
| FXIa-FVIII | LGAVELSWDYMQSDLGELPVDARFPPRVPKSFPFNTSVVYKKTLFVEFTDHLFNIAKPRPPW |
| BDD9- | MGLLGPTIQAEVYDTVVITLKNMASHPVSLHAVGVSYWKASEGAEYDDQTSQREKEDDKVF |
| AE864 | PGGSHTYVWQVLKENGPMASDPLCLTYSYLSHVDLVKDLNSGLIGALLVCREGSLAKEKTQ |
| | TLHKFILLFAVFDEGKSWHSETKNSLMQDRDAASARAWPKMHTVNGYVNRSLPGLIGCHRK |
| | SVYWHVIGMGTTPEVHSIFLEGHTFLVRNHRQASLEISPITFLTAQTLLMDLGQFLLFCHISSH |
| | QHDGMEAYVKVDSCPEEPQLRMKNNEEAEDYDDDLTDSEMDVVRFDDDNSPSFIQIRSVAK |
| | KHPKTWVHYIAAEEEDWDYAPLVLAPDDRSYKSQYLNNGPQRIGRKYKKVRFMAYTDETF |
| | KTREAIQHESGILGPLLYGEVGDTLLIIFKNQASRPYNIYPHGITDVRPLYSRRLPKGVKHLKDF |
| | PILPGEIFKYKWTVTVEDGPTKSDPRCLTRYYSSFVNMERDLASGLIGPLLICYKESVDQRGNQ |
| | IMSDKRNVILFSVFDENRSWYLTENIQRFLPNPAGVQLEDPEFQASNIMHSINGYVFDSLQLSV |
| | CLHEVAYWYILSIGAQTDFLSVFFSGYTFKHKMVYEDTLTLFPFSGETVFMSMENPGLWILGC |
| | HNSDFRNRGMTALLKVSSCDKNTGDYYEDSYEDISAYLLSKNNAIEPRSFSQNPPVLKRHQRE |
| | ITRTTLQSDQEEIDYDDTISVEMKKEDFDIYDEDENQSPRSFQKKTRHYFIAAVERLWDYGMS |
| | SSPHVLRNRAQSGSVPQFKKVVFQEFTDGSFTQPLYRGELNEHLGLLGPYIRAEVEDNIMVTF |
| | RNQASRPYSFYSSLISYEEDQRQGAEPRKNFVKPNETKTYFWKVQHHMAPTKDEFDCKAWA |
| | YFSDVDLEKDVHSGLIGPLLVCITINTLNPAHGRQVTVQEFALFFTIFDETKSWYFTENMERNC |
| | RAPCNIQMEDPTFKENYRFHAINGYIMDTLPGLVMAQDQRIRWYLLSMGSNENIHSIHFSGHV |
| | FTVRKKEEYKMALYNLYPGVFETVEMLPSKAGIWRVECLIGEHLHAGMSTLFLVYSNKCQTP |
| | LGMASGHIRDFQITASGQYGQWAPKLARLHYSGSINAWSTKEPFSWIKVDLLAPMIIHGIKTQ |
| | GARQKFSSLYISQFIIMYSLDGKKWQTYRGNSTGTLMVFFGNVDSSGIKHNIFNPPIIARYIRLH |
| | PTHYSIRSTLRMELMGCDLNSCSMPLGMESKAISDAQITASSYFTNMFATWSPSKARLHLQGR |
| | SNAWRPQVNNPKEWLQVDFQKTMKVTGVTTQGVKSLLTSMYVKEFLISSSQDGHQWTLFFQ |
| | NGKVKVFQGNQDSFTPVVNSLDPPLLTRYLRIHPQSWVHQIALRMEVLGCEAQDLYGGSPAG |
| | SPTSTEEGTSESATPESGPGTSTEPSEGSAPGSPAGSPTSTEEGTSTEPSEGSAPGTSTEPSEGSAP |
| | GTSESATPESGPGSEPATSGSETPGSEPATSGSETPGSPAGSPTSTEEGTSESATPESGPGTSTEPS |
| | EGSAPGTSTEPSEGSAPGSPAGSPTSTEEGTSTEPSEGSAPGTSTEPSEGSAPGTSESATPESGPG |
| | TSTEPSEGSAPGTSESATPESGPGSEPATSGSETPGTSTEPSEGSAPGTSTEPSEGSAPGTSESATP |
| | ESGPGTSESATPESGPGSPAGSPTSTEEGTSESATPESGPGSEPATSGSETPGTSESATPESGPGT |
| | STEPSEGSAPGTSTEPSEGSAPGTSTEPSEGSAPGTSTEPSEGSAPGTSTEPSEGSAPGTSTEPSEG |
| | SAPGSPAGSPTSTEEGTSTEPSEGSAPGTSESATPESGPGSEPATSGSETPGTSESATPESGPGSE |
| | PATSGSETPGTSESATPESGPGTSTEPSEGSAPGTSESATPESGPGSPAGSPTSTEEGSPAGSPTS |
| | TEEGSPAGSPTSTEEGTSESATPESGPGTSTEPSEGSAPGTSESATPESGPGSEPATSGSETPGTS |
| | ESATPESGPGSEPATSGSETPGTSESATPESGPGTSTEPSEGSAPGSPAGSPTSTEEGTSESATPES |
| | GPGSEPATSGSETPGTSESATPESGPGSPAGSPTSTEEGSPAGSPTSTEEGTSTEPSEGSAPGTSE |
| | SATPESGPGTSESATPESGPGTSESATPESGPGSEPATSGSETPGSEPATSGSETPGSPAGSPTST |
| | EEGTSTEPSEGSAPGTSTEPSEGSAPGSEPATSGSETPGTSESATPESGPGTSTEPSEGSAP |
| FVIII | ATRRYYLGAVELSWDYMQSDLGELPVDARFPPRVPKSFPFNTSVVYKKTLFVEFTDHLFNIA |
| BDD9- | KPRPPWMGLLGPTIQAEVYDTVVITLKNMASHPVSLHAVGVSYWKASEGAEYDDQTSQREK |
| FXIa- | EDDKVFPGGSHTYVWQVLKENGPMASDPLCLTYSYLSHVDLVKDLNSGLIGALLVCREGSL |
| AG288_2 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQREITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYKLTRAETGPGASPGTSSTGSPGASPGTSSTGSPGTPGSGTASSSPGSSTPSGATGSPGTPG |
| | SGTASSSPGSSTPSGATGSPGTPGSGTASSSPGSSTPSGATGSPGSSTPSGATGSPGSSPSASTGT |
| | GPGSSPSASTGTGPGASPGTSSTGSPGTPGSGTASSSPGSSTPSGATGSPGSSPSASTGTGPGSSP |
| | SASTGTGPGASPGTSSTGSPGASPGTSSTGSPGSSTPSGATGSPGSSPSASTGTGPGASPGTSSTG |
| | SPGSSPSASTGTGPGTPGSGTASSSPGSSTPSGATGS |
| FVIII | ATRRYYLGAVELSWDYMQSDLGELPVDARFPPRVPKSFPFNTSVVYKKTLFVEFTDHLFNIA |
| BDD9- | KPRPPWMGLLGPTIQAEVYDTVVITLKNMASHPVSLHAVGVSYWKASEGAEYDDQTSQREK |
| FXIa- | EDDKVFPGGSHTYVWQVLKENGPMASDPLCLTYSYLSHVDLVKDLNSGLIGALLVCREGSL |
| AG864 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQREITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYKLTRAETGGASPGTSSTGSPGSSPSASTGTGPGSSPSASTGTGPGTPGSGTASSSPGSSTP |
| | SGATGSPGSSPSASTGTGPGASPGTSSTGSPGTPGSGTASSSPGSSTPSGATGSPGTPGSGTASSS |
| | PGASPGTSSTGSPGASPGTSSTGSPGTPGSGTASSSPGSSTPSGATGSPGASPGTSSTGSPGTPGS |
| | GTASSSPGSSTPSGATGSPGSSPSASTGTGPGSSPSASTGTGPGSSTPSGATGSPGSSTPSGATGS |
| | PGASPGTSSTGSPGASPGTSSTGSPGASPGTSSTGSPGTPGSGTASSSPGASPGTSSTGSPGASPG |
| | TSSTGSPGASPGTSSTGSPGSSPSASTGTGPGTPGSGTASSSPGASPGTSSTGSPGASPGTSSTGS |
| | PGASPGTSSTGSPGSSTPSGATGSPGSSTPSGATGSPGASPGTSSTGSPGTPGSGTASSSPGSSTP |
| | SGATGSPGSSTPSGATGSPGSSTPSGATGSPGSSPSASTGTGPGASPGTSSTGSPGASPGTSSTGS |
| | PGTPGSGTASSSPGASPGTSSTGSPGASPGTSSTGSPGASPGTSSTGSPGASPGTSSTGSPGTPGS |
| | GTASSSPGSSTPSGATGSPGTPGSGTASSSPGSSTPSGATGSPGTPGSGTASSSPGSSTPSGATGS |
| | PGSSTPSGATGSPGSSPSASTGTGPGSSPSASTGTGPGASPGTSSTGSPGTPGSGTASSSPGSSTP |
| | SGATGSPGSSPSASTGTGPGSSPSASTGTGPGASPGTSSTGSPGASPGTSSTGSPGSSTPSGATGS |
| | PGSSPSASTGTGPGASPGTSSTGSPGSSPSASTGTGPGTPGSGTASSSPGSSTPSGATGSPGSSTP |
| | SGATGSPGASPGTSSTGSP |
| FVIII | ATRRYYLGAVELSWDYMQSDLGELPVDARFPPRVPKSFPFNTSVVYKKTLFVEFTDHLFNIA |
| BDD9 (1- | KPRPPWMGLLGPTIQAEVYDTVVITLKNMASHPVSLHAVGVSYWKASEGAEYDDQTSQREK |
| 745) | EDDKVFPGGSHTYVWQVLKENGPMASDPLCLTYSYLSHVDLVKDLNSGLIGALLVCREGSL |
| AG288_2- | AKEKTQTLHKFILLFAVFDEGKSWHSETKNSLMQDRDAASARAWPKMHTVNGYVNRSLPGL |
| (1640- | IGCHRKSVYWHVIGMGTTPEVHSIFLEGHTFLVRNHRQASLEISPITFLTAQTLLMDLGQFLLF |
| Y2332)- | CHISSHQHDGMEAYVKVDSCPEEPQLRMKNNEEAEDYDDDLTDSEMDVVRFDDDNSPSFIQI |
| FXIa- | RSVAKKHPKTWVHYIAAEEEDWDYAPLVLAPDDRSYKSQYLNNGPQRIGRKYKKVRFMAY |
| AG864 | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNGPG |
| | ASPGTSSTGSPGASPGTSSTGSPGTPGSGTASSSPGSSTPSGATGSPGTPGSGTASSSPGSSTPSG |
| | ATGSPGTPGSGTASSSPGSSTPSGATGSPGSSTPSGATGSPGSSPSASTGTGPGSSPSASTGTGPG |
| | ASPGTSSTGSPGTPGSGTASSSPGSSTPSGATGSPGSSPSASTGTGPGSSPSASTGTGPGASPGTS |
| | STGSPGASPGTSSTGSPGSSTPSGATGSPGSSPSASTGTGPGASPGTSSTGSPGSSPSASTGTGPG |
| | TPGSGTASSSPGSSTPSGATGSPPVLKRHQREITRTTLQSDQEEIDYDDTISVEMKKEDFDIYDE |
| | DENQSPRSFQKKTRHYFIAAVERLWDYGMSSSPHVLRNRAQSGSVPQFKKVVFQEFTDGSFT |
| | QPLYRGELNEHLGLLGPYIRAEVEDNIMVTFRNQASRPYSFYSSLISYEEDQRQGAEPRKNFV |
| | KPNETKTYFWKVQHHMAPTKDEFDCKAWAYFSDVDLEKDVHSGLIGPLLVCHTNTLNPAH |
| | GRQVTVQEFALFFTIFDETKSWYFTENMERNCRAPCNIQMEDPTFKENYRFHAINGYIMDTLP |
| | GLVMAQDQRIRWYLLSMGSNENIHSIHFSGHVFTVRKKEEYKMALYNLYPGVFETVEMLPS |
| | KAGIWRVECLIGEHLHAGMSTLFLVYSNKCQTPLGMASGHIRDFQITASGQYGQWAPKLARL |
| | HYSGSINAWSTKEPFSWIKVDLLAPMIIHGIKTQGARQKFSSLYISQFIIMYSLDGKKWQTYRG |
| | NSTGTLMVFFGNVDSSGIKHNIFNPPIIARYIRLHPTHYSIRSTLRMELMGCDLNSCSMPLGME |
| | SKAISDAQITASSYFTNMFATWSPSKARLHLQGRSNAWRPQVNNPKEWLQVDFQKTMKVTG |
| | VTTQGVKSLLTSMYVKEFLISSSQDGHQWTLFFQNGKVKVFQGNQDSFTPVVNSLDPPLLTR |
| | YLRIHPQSWVHQIALRMEVLGCEAQDLYKLTRAETGGASPGTSSTGSPGSSPSASTGTGPGSS |
| | PSASTGTGPGTPGSGTASSSPGSSTPSGATGSPGSSPSASTGTGPGASPGTSSTGSPGTPGSGTAS |
| | SSPGSSTPSGATGSPGTPGSGTASSSPGASPGTSSTGSPGASPGTSSTGSPGTPGSGTASSSPGSS |
| | TPSGATGSPGASPGTSSTGSPGTPGSGTASSSPGSSTPSGATGSPGSSPSASTGTGPGSSPSASTG |
| | TGPGSSTPSGATGSPGSSTPSGATGSPGASPGTSSTGSPGASPGTSSTGSPGASPGTSSTGSPGTP |
| | GSGTASSSPGASPGTSSTGSPGASPGTSSTGSPGASPGTSSTGSPGSSPSASTGTGPGTPGSGTAS |
| | SSPGASPGTSSTGSPGASPGTSSTGSPGASPGTSSTGSPGSSTPSGATGSPGSSTPSGATGSPGAS |
| | PGTSSTGSPGTPGSGTASSSPGSSTPSGATGSPGSSTPSGATGSPGSSTPSGATGSPGSSPSASTG |
| | TGPGASPGTSSTGSPGASPGTSSTGSPGTPGSGTASSSPGASPGTSSTGSPGASPGTSSTGSPGAS |
| | PGTSSTGSPGASPGTSSTGSPGTPGSGTASSSPGSSTPSGATGSPGTPGSGTASSSPGSSTPSGAT |
| | GSPGTPGSGTASSSPGSSTPSGATGSPGSSTPSGATGSPGSSPSASTGTGPGSSPSASTGTGPGAS |
| | PGTSSTGSPGTPGSGTASSSPGSSTPSGATGSPGSSPSASTGTGPGSSPSASTGTGPGASPGTSST |
| | GSPGASPGTSSTGSPGSSTPSGATGSPGSSPSASTGTGPGASPGTSSTGSPGSSPSASTGTGPGTP |
| | GSGTASSSPGSSTPSGATGSPGSSTPSGATGSPGASPGTSSTGSP |
| FVIII | ATRRYYLGAVELSWDYMQSDLGELPVDARFPPRVPKSFPFNTSVVYKKTLFVEFTDHLFNIA |
| BDD9 (1- | KPRPPWMGLLGPTIQAEVYDTVVITLKNMASHPVSLHAVGVSYWKASEGAEYDDQTSQREK |
| 743) | EDDKVFPGGSHTYVWQVLKENGPMASDPLCLTYSYLSHVDLVKDLNSGLIGALLVCREGSL |
| AG288_2- | AKEKTQTLHKFILLFAVFDEGKSWHSETKNSLMQDRDAASARAWPKMHTVNGYVNRSLPGL |
| (1638- | IGCHRKSVYWHVIGMGTTPEVHSIFLEGHTFLVRNHRQASLEISPITFLTAQTLLMDLGQFLLF |
| Y2332)- | CHISSHQHDGMEAYVKVDSCPEEPQLRMKNNEEAEDYDDDLTDSEMDVVRFDDDNSPSFIQI |
| FXIa- | RSVAKKHPKTWVHYIAAEEEDWDYAPLVLAPDDRSYKSQYLNNGPQRIGRKYKKVRFMAY |
| AG864 | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSGPGASP |
| | GTSSTGSPGASPGTSSTGSPGTPGSGTASSSPGSSTPSGATGSPGTPGSGTASSSPGSSTPSGATG |
| | SPGTPGSGTASSSPGSSTPSGATGSPGSSTPSGATGSPGSSPSASTGTGPGSSPSASTGTGPGASP |
| | GTSSTGSPGTPGSGTASSSPGSSTPSGATGSPGSSPSASTGTGPGSSPSASTGTGPGASPGTSSTG |
| | SPGASPGTSSTGSPGSSTPSGATGSPGSSPSASTGTGPGASPGTSSTGSPGSSPSASTGTGPGTPG |
| | SGTASSSPGSSTPSGATGSQNPPVLKRHQREITRTTLQSDQEEIDYDDTISVEMKKEDFDIYDE |
| | DENQSPRSFQKKTRHYFIAAVERLWDYGMSSSPHVLRNRAQSGSVPQFKKVVFQEFTDGSFT |
| | QPLYRGELNEHLGLLGPYIRAEVEDNIMVTFRNQASRPYSFYSSLISYEEDQRQGAEPRKNFV |
| | KPNETKTYFWKVQHHMAPTKDEFDCKAWAYFSDVDLEKDVHSGLIGPLLVCHTNTLNPAH |
| | GRQVTVQEFALFFTIFDETKSWYFTENMERNCRAPCNIQMEDPTFKENYRFHAINGYIMDTLP |
| | GLVMAQDQRIRWYLLSMGSNENIHSIHFSGHVFTVRKKEEYKMALYNLYPGVFETVEMLPS |
| | KAGIWRVECLIGEHLHAGMSTLFLVYSNKCQTPLGMASGHIRDFQITASGQYGQWAPKLARL |
| | HYSGSINAWSTKEPFSWIKVDLLAPMIIHGIKTQGARQKFSSLYISQFIIMYSLDGKKWQTYRG |
| | NSTGTLMVFFGNVDSSGIKHNIFNPPIIARYIRLHPTHYSIRSTLRMELMGCDLNSCSMPLGME |
| | SKAISDAQITASSYFTNMFATWSPSKARLHLQGRSNAWRPQVNNPKEWLQVDFQKTMKVTG |
| | VTTQGVKSLLTSMYVKEFLISSSQDGHQWTLFFQNGKVKVFQGNQDSFTPVVNSLDPPLLTR |
| | YLRIHPQSWVHQIALRMEVLGCEAQDLYKLTRAETGGASPGTSSTGSPGSSPSASTGTGPGSS |
| | PSASTGTGPGTPGSGTASSSPGSSTPSGATGSPGSSPSASTGTGPGASPGTSSTGSPGTPGSGTAS |
| | SSPGSSTPSGATGSPGTPGSGTASSSPGASPGTSSTGSPGASPGTSSTGSPGTPGSGTASSSPGSS |
| | TPSGATGSPGASPGTSSTGSPGTPGSGTASSSPGSSTPSGATGSPGSSPSASTGTGPGSSPSASTG |
| | TGPGSSTPSGATGSPGSSTPSGATGSPGASPGTSSTGSPGASPGTSSTGSPGASPGTSSTGSPGTP |
| | GSGTASSSPGASPGTSSTGSPGASPGTSSTGSPGASPGTSSTGSPGSSPSASTGTGPGTPGSGTAS |
| | SSPGASPGTSSTGSPGASPGTSSTGSPGASPGTSSTGSPGSSTPSGATGSPGSSTPSGATGSPGAS |
| | PGTSSTGSPGTPGSGTASSSPGSSTPSGATGSPGSSTPSGATGSPGSSTPSGATGSPGSSPSASTG |
| | TGPGASPGTSSTGSPGASPGTSSTGSPGTPGSGTASSSPGASPGTSSTGSPGASPGTSSTGSPGAS |
| | PGTSSTGSPGASPGTSSTGSPGTPGSGTASSSPGSSTPSGATGSPGTPGSGTASSSPGSSTPSGAT |
| | GSPGTPGSGTASSSPGSSTPSGATGSPGSSTPSGATGSPGSSPSASTGTGPGSSPSASTGTGPGAS |
| | PGTSSTGSPGTPGSGTASSSPGSSTPSGATGSPGSSPSASTGTGPGSSPSASTGTGPGASPGTSST |
| | GSPGASPGTSSTGSPGSSTPSGATGSPGSSPSASTGTGPGASPGTSSTGSPGSSPSASTGTGPGTP |
| | GSGTASSSPGSSTPSGATGSPGSSTPSGATGSPGASPGTSSTGSP |
| BDD10 (1- | ATRRYYLGAVELSWDYMQSDLGELPVDARFPPRVPKSFPFNTSVVYKKTLFVEFTDHLFNIA |
| 745) | KPRPPWMGLLGPTIQAEVYDTVVITLKNMASHPVSLHAVGVSYWKASEGAEYDDQTSQREK |
| AG288_2- | EDDKVFPGGSHTYVWQVLKENGPMASDPLCLTYSYLSHVDLVKDLNSGLIGALLVCREGSL |
| (1640- | AKEKTQTLHKFILLFAVFDEGKSWHSETKNSLMQDRDAASARAWPKMHTVNGYVNRSLPGL |
| Y2332)- | IGCHRKSVYWHVIGMGTTPEVHSIFLEGHTFLVRNHRQASLEISPITFLTAQTLLMDLGQFLLF |
| FXIa- | CHISSHQHDGMEAYVKVDSCPEEPQLRMKNNEEAEDYDDDLTDSEMDVVRFDDDNSPSFIQI |
| AG864 | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNGPG |
| | ASPGTSSTGSPGASPGTSSTGSPGTPGSGTASSSPGSSTPSGATGSPGTPGSGTASSSPGSSTPSG |
| | ATGSPGTPGSGTASSSPGSSTPSGATGSPGSSTPSGATGSPGSSPSASTGTGPGSSPSASTGTGPG |
| | ASPGTSSTGSPGTPGSGTASSSPGSSTPSGATGSPGSSPSASTGTGPGSSPSASTGTGPGASPGTS |
| | STGSPGASPGTSSTGSPGSSTPSGATGSPGSSPSASTGTGPGASPGTSSTGSPGSSPSASTGTGPG |
| | TPGSGTASSSPGSSTPSGATGSPPVLKRHQAEITRTTLQSDQEEIDYDDTISVEMKKEDFDIYDE |
| | DENQSPRSFQKKTRHYFIAAVERLWDYGMSSSPHVLRNRAQSGSVPQFKKVVFQEFTDGSFT |
| | QPLYRGELNEHLGLLGPYIRAEVEDNIMVTFRNQASRPYSFYSSLISYEEDQRQGAEPRKNFV |
| | KPNETKTYFWKVQHHMAPTKDEFDCKAWAYFSDVDLEKDVHSGLIGPLLVCHTNTLNPAH |
| | GRQVTVQEFALFFTIFDETKSWYFTENMERNCRAPCNIQMEDPTFKENYRFHAINGYIMDTLP |
| | GLVMAQDQRIRWYLLSMGSNENIHSIHFSGHVFTVRKKEEYKMALYNLYPGVFETVEMLPS |
| | KAGIWRVECLIGEHLHAGMSTLFLVYSNKCQTPLGMASGHIRDFQITASGQYGQWAPKLARL |
| | HYSGSINAWSTKEPFSWIKVDLLAPMIIHGIKTQGARQKFSSLYISQFIIMYSLDGKKWQTYRG |
| | NSTGTLMVFFGNVDSSGIKHNIFNPPIIARYIRLHPTHYSIRSTLRMELMGCDLNSCSMPLGME |
| | SKAISDAQITASSYFTNMFATWSPSKARLHLQGRSNAWRPQVNNPKEWLQVDFQKTMKVTG |
| | VTTQGVKSLLTSMYVKEFLISSSQDGHQWTLFFQNGKVKVFQGNQDSFTPVVNSLDPPLLTR |
| | YLRIHPQSWVHQIALRMEVLGCEAQDLYKLTRAETGGASPGTSSTGSPGSSPSASTGTGPGSS |
| | PSASTGTGPGTPGSGTASSSPGSSTPSGATGSPGSSPSASTGTGPGASPGTSSTGSPGTPGSGTAS |
| | SSPGSSTPSGATGSPGTPGSGTASSSPGASPGTSSTGSPGASPGTSSTGSPGTPGSGTASSSPGSS |
| | TPSGATGSPGASPGTSSTGSPGTPGSGTASSSPGSSTPSGATGSPGSSPSASTGTGPGSSPSASTG |
| | TGPGSSTPSGATGSPGSSTPSGATGSPGASPGTSSTGSPGASPGTSSTGSPGASPGTSSTGSPGTP |
| | GSGTASSSPGASPGTSSTGSPGASPGTSSTGSPGASPGTSSTGSPGSSPSASTGTGPGTPGSGTAS |
| | SSPGASPGTSSTGSPGASPGTSSTGSPGASPGTSSTGSPGSSTPSGATGSPGSSTPSGATGSPGAS |
| | PGTSSTGSPGTPGSGTASSSPGSSTPSGATGSPGSSTPSGATGSPGSSTPSGATGSPGSSPSASTG |
| | TGPGASPGTSSTGSPGASPGTSSTGSPGTPGSGTASSSPGASPGTSSTGSPGASPGTSSTGSPGAS |
| | PGTSSTGSPGASPGTSSTGSPGTPGSGTASSSPGSSTPSGATGSPGTPGSGTASSSPGSSTPSGAT |
| | GSPGTPGSGTASSSPGSSTPSGATGSPGSSTPSGATGSPGSSPSASTGTGPGSSPSASTGTGPGAS |
| | PGTSSTGSPGTPGSGTASSSPGSSTPSGATGSPGSSPSASTGTGPGSSPSASTGTGPGASPGTSST |
| | GSPGASPGTSSTGSPGSSTPSGATGSPGSSPSASTGTGPGASPGTSSTGSPGSSPSASTGTGPGTP |
| | GSGTASSSPGSSTPSGATGSPGSSTPSGATGSPGASPGTSSTGSP |
| FVIII | ATRRYYLGAVELSWDYMQSDLGELPVDARFPPRVPKSFPFNTSVVYKKTLFVEFTDHLFNIA |
| BDD10- | KPRPPWMGLLGPTIQAEVYDTVVITLKNMASHPVSLHAVGVSYWKASEGAEYDDQTSQREK |
| FXIa- | EDDKVFPGGSHTYVWQVLKENGPMASDPLCLTYSYLSHVDLVKDLNSGLIGALLVCREGSL |
| AG288_2 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQAEITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYKLTRAETGAGSPGAETAPGASPGTSSTGSPGASPGTSSTGSPGTPGSGTASSSPGSSTPSG |
| | ATGSPGTPGSGTASSSPGSSTPSGATGSPGTPGSGTASSSPGSSTPSGATGSPGSSTPSGATGSPG |
| | SSPSASTGTGPGSSPSASTGTGPGASPGTSSTGSPGTPGSGTASSSPGSSTPSGATGSPGSSPSAS |
| | TGTGPGSSPSASTGTGPGASPGTSSTGSPGASPGTSSTGSPGSSTPSGATGSPGSSPSASTGTGPG |
| | ASPGTSSTGSPGSSPSASTGTGPGTPGSGTASSSPGSSTPSGATGSGAETAEQKLISEEDLSPATG |
| FVIII | ATRRYYLGAVELSWDYMQSDLGELPVDARFPPRVPKSFPFNTSVVYKKTLFVEFTDHLFNIA |
| BDD10- | KPRPPWMGLLGPTIQAEVYDTVVITLKNMASHPVSLHAVGVSYWKASEGAEYDDQTSQREK |
| FXIa- | EDDKVFPGGSHTYVWQVLKENGPMASDPLCLTYSYLSHVDLVKDLNSGLIGALLVCREGSL |
| AG864 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQAEITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYKLTRAETGGASPGTSSTGSPGSSPSASTGTGPGSSPSASTGTGPGTPGSGTASSSPGSSTP |
| | SGATGSPGSSPSASTGTGPGASPGTSSTGSPGTPGSGTASSSPGSSTPSGATGSPGTPGSGTASSS |
| | PGASPGTSSTGSPGASPGTSSTGSPGTPGSGTASSSPGSSTPSGATGSPGASPGTSSTGSPGTPGS |
| | GTASSSPGSSTPSGATGSPGSSPSASTGTGPGSSPSASTGTGPGSSTPSGATGSPGSSTPSGATGS |
| | PGASPGTSSTGSPGASPGTSSTGSPGASPGTSSTGSPGTPGSGTASSSPGASPGTSSTGSPGASPG |
| | TSSTGSPGASPGTSSTGSPGSSPSASTGTGPGTPGSGTASSSPGASPGTSSTGSPGASPGTSSTGS |
| | PGASPGTSSTGSPGSSTPSGATGSPGSSTPSGATGSPGASPGTSSTGSPGTPGSGTASSSPGSSTP |
| | SGATGSPGSSTPSGATGSPGSSTPSGATGSPGSSPSASTGTGPGASPGTSSTGSPGASPGTSSTGS |
| | PGTPGSGTASSSPGASPGTSSTGSPGASPGTSSTGSPGASPGTSSTGSPGASPGTSSTGSPGTPGS |
| | GTASSSPGSSTPSGATGSPGTPGSGTASSSPGSSTPSGATGSPGTPGSGTASSSPGSSTPSGATGS |
| | PGSSTPSGATGSPGSSPSASTGTGPGSSPSASTGTGPGASPGTSSTGSPGTPGSGTASSSPGSSTP |
| | SGATGSPGSSPSASTGTGPGSSPSASTGTGPGASPGTSSTGSPGASPGTSSTGSPGSSTPSGATGS |
| | PGSSPSASTGTGPGASPGTSSTGSPGSSPSASTGTGPGTPGSGTASSSPGSSTPSGATGSPGSSTP |
| | SGATGSPGASPGTSSTGSP |
| FVIII | ATRRYYLGAVELSWDYMQSDLGELPVDARFPPRVPKSFPFNTSVVYKKTLFVEFTDHLFNIA |
| BDD10- | KPRPPWMGLLGPTIQAEVYDTVVITLKNMASHPVSLHAVGVSYWKASEGAEYDDQTSQREK |
| FXIa- | EDDKVFPGGSHTYVWQVLKENGPMASDPLCLTYSYLSHVDLVKDLNSGLIGALLVCREGSL |
| AE864 | AKEKTQTLHKFILLFAVFDEGKSWHSETKNSLMQDRDAASARAWPKMHTVNGYVNRSLPGL |
| | IGCHRKSVYWHVIGMGTTPEVHSIFLEGHTFLVRNHRQASLEISPITFLTAQTLLMDLGQFLLF |
| | CHISSHQHDGMEAYVKVDSCPEEPQLRMKNNEEAEDYDDDLTDSEMDVVRFDDDNSPSFIQI |
| | RSVAKKHPKTWVHYIAAEEEDWDYAPLVLAPDDRSYKSQYLNNGPQRIGRKYKKVRFMAY |
| | TDETFKTREAIQHESGILGPLLYGEVGDTLLIIFKNQASRPYNIYPHGITDVRPLYSRRLPKGVK |
| | HLKDFPILPGEIFKYKWTVTVEDGPTKSDPRCLTRYYSSFVNMERDLASGLIGPLLICYKESVD |
| | QRGNQIMSDKRNVILFSVFDENRSWYLTENIQRFLPNPAGVQLEDPEFQASNIMHSINGYVFD |
| | SLQLSVCLHEVAYWYILSIGAQTDFLSVFFSGYTFKHKMVYEDTLTLFPFSGETVFMSMENPG |
| | LWILGCHNSDFRNRGMTALLKVSSCDKNTGDYYEDSYEDISAYLLSKNNAIEPRSFSQNPPVL |
| | KRHQAEITRTTLQSDQEEIDYDDTISVEMKKEDFDIYDEDENQSPRSFQKKTRHYFIAAVERL |
| | WDYGMSSSPHVLRNRAQSGSVPQFKKVVFQEFTDGSFTQPLYRGELNEHLGLLGPYIRAEVE |
| | DNIMVTFRNQASRPYSFYSSLISYEEDQRQGAEPRKNFVKPNETKTYFWKVQHHMAPTKDEF |
| | DCKAWAYFSDVDLEKDVHSGLIGPLLVCHTNTLNPAHGRQVTVQEFALFFTIFDETKSWYFT |
| | ENMERNCRAPCNIQMEDPTFKENYRFHAINGYIMDTLPGLVMAQDQRIRWYLLSMGSNENIH |
| | SIHFSGHVFTVRKKEEYKMALYNLYPGVFETVEMLPSKAGIWRVECLIGEHLHAGMSTLFLV |
| | YSNKCQTPLGMASGHIRDFQITASGQYGQWAPKLARLHYSGSINAWSTKEPFSWIKVDLLAP |
| | MIIHGIKTQGARQKFSSLYISQFIIMYSLDGKKWQTYRGNSTGTLMVFFGNVDSSGIKHNIFNP |
| | PIIARYIRLHPTHYSIRSTLRMELMGCDLNSCSMPLGMESKAISDAQITASSYFTNMFATWSPS |
| | KARLHLQGRSNAWRPQVNNPKEWLQVDFQKTMKVTGVTTQGVKSLLTSMYVKEFLISSSQD |
| | GHQWTLFFQNGKVKVFQGNQDSFTPVVNSLDPPLLTRYLRIHPQSWVHQIALRMEVLGCEA |
| | QDLYKLTRAETGGSPAGSPTSTEEGTSESATPESGPGTSTEPSEGSAPGSPAGSPTSTEEGTSTE |
| | PSEGSAPGTSTEPSEGSAPGTSESATPESGPGSEPATSGSETPGSEPATSGSETPGSPAGSPTSTE |
| | EGTSESATPESGPGTSTEPSEGSAPGTSTEPSEGSAPGSPAGSPTSTEEGTSTEPSEGSAPGTSTE |
| | PSEGSAPGTSESATPESGPGTSTEPSEGSAPGTSESATPESGPGSEPATSGSETPGTSTEPSEGSA |
| | PGTSTEPSEGSAPGTSESATPESGPGTSESATPESGPGSPAGSPTSTEEGTSESATPESGPGSEPA |
| | TSGSETPGTSESATPESGPGTSTEPSEGSAPGTSTEPSEGSAPGTSTEPSEGSAPGTSTEPSEGSAP |
| | GTSTEPSEGSAPGTSTEPSEGSAPGSPAGSPTSTEEGTSTEPSEGSAPGTSESATPESGPGSEPAT |
| | SGSETPGTSESATPESGPGSEPATSGSETPGTSESATPESGPGTSTEPSEGSAPGTSESATPESGP |
| | GSPAGSPTSTEEGSPAGSPTSTEEGSPAGSPTSTEEGTSESATPESGPGTSTEPSEGSAPGTSESA |
| | TPESGPGSEPATSGSETPGTSESATPESGPGSEPATSGSETPGTSESATPESGPGTSTEPSEGSAP |
| | GSPAGSPTSTEEGTSESATPESGPGSEPATSGSETPGTSESATPESGPGSPAGSPTSTEEGSPAGS |
| | PTSTEEGTSTEPSEGSAPGTSESATPESGPGTSESATPESGPGTSESATPESGPGSEPATSGSETP |
| | GSEPATSGSETPGSPAGSPTSTEEGTSTEPSEGSAPGTSTEPSEGSAPGSEPATSGSETPGTSESA |
| | TPESGPGTSTEPSEGSAP |

*Sequence name reflects N- to C-terminus configuration of the FVIII variant and XTEN components: signal peptide (SP); linker (L); cleavage sequence (CS) may be denoted by protease name active on the sequence, and XTEN components by family name and length, with insertion points for components denoted by FVIII amino acid and numbered positions adjacent to the inserted sequence or A1 being the N-terminus and Y2332 being the C-terminus of the FVIII.

TABLE 31
FVIII amino acid sequences

| Name | |
|---|---|
| (source) | Amino Acid Sequence |
| FVIII BDD-10 | ATRRYYLGAVELSWDYMQSDLGELPVDARFPPRVPKSFPFNTSVVYKKTLFVEFTDHLF |
| | NIAKPRPPWMGLLGPTIQAEVYDTVVITLKNMASHPVSLHAVGVSYWKASEGAEYDDQ |
| | TSQREKEDDKVFPGGSHTYVWQVLKENGPMASDPLCLTYSYLSHVDLVKDLNSGLIGA |
| | LLVCREGSLAKEKTQTLHKFILLFAVFDEGKSWHSETKNSLMQDRDAASARAWPKMHT |
| | VNGYVNRSLPGLIGCHRKSVYWHVIGMGTTPEVHSIFLEGHTFLVRNHRQASLEISPITFL |
| | TAQTLLMDLGQFLLFCHISSHQHDGMEAYVKVDSCPEEPQLRMKNNEEAEDYDDDLTD |
| | SEMDVVRFDDDNSPSFIQIRSVAKKHPKTWVHYIAAEEEDWDYAPLVLAPDDRSYKSQ |
| | YLNNGPQRIGRKYKKVRFMAYTDETFKTREAIQHESGILGPLLYGEVGDTLLIIFKNQAS |
| | RPYNIYPHGITDVRPLYSRRLPKGVKHLKDFPILPGEIFKYKWTVTVEDGPTKSDPRCLT |
| | RYYSSFVNMERDLASGLIGPLLICYKESVDQRGNQIMSDKRNVILFSVFDENRSWYLTEN |
| | IQRFLPNPAGVQLEDPEFQASNIMHSINGYVFDSLQLSVCLHEVAYWYILSIGAQTDFLS |
| | VFFSGYTFKHKMVYEDTLTLFPFSGETVFMSMENPGLWILGCHNSDFRNRGMTALLKV |
| | SSCDKNTGDYYEDSYEDISAYLLSKNNAIEPRSFSQNPPVLKRHQAEITRTTLQSDQEEID |
| | YDDTISVEMKKEDFDIYDEDENQSPRSFQKKTRHYFIAAVERLWDYGMSSSPHVLRNRA |
| | QSGSVPQFKKVVFQEFTDGSFTQPLYRGELNEHLGLLGPYIRAEVEDNIMVTFRNQASRP |
| | YSFYSSLISYEEDQRQGAEPRKNFVKPNETKTYFWKVQHHMAPTKDEFDCKAWAYFSD |
| | VDLEKDVHSGLIGPLLVCHTNTLNPAHGRQVTVQEFALFFTIFDETKSWYFTENMERNC |
| | RAPCNIQMEDPTFKENYRFHAINGYIMDTLPGLVMAQDQRIRWYLLSMGSNENIHSIHFS |
| | GHVFTVRKKEEYKMALYNLYPGVFETVEMLPSKAGIWRVECLIGEHLHAGMSTLFLVY |
| | SNKCQTPLGMASGHIRDFQITASGQYGQWAPKLARLHYSGSINAWSTKEPFSWIKVDLL |
| | APMIIHGIKTQGARQKFSSLYISQFIIMYSLDGKKWQTYRGNSTGTLMVFFGNVDSSGIK |
| | HNIFNPPIIARYIRLHPTHYSIRSTLRMELMGCDLNSCSMPLGMESKAISDAQITASSYFTN |
| | MFATWSPSKARLHLQGRSNAWRPQVNNPKEWLQVDFQKTMKVTGVTTQGVKSLLTS |
| | MYVKEFLISSSQDGHQWTLFFQNGKVKVFQGNQDSFTPVVNSLDPPLLTRYLRIHPQSW |
| | VHQIALRMEVLGCEAQDLY |
| FVIII BDD-11 | ATRATRRYYLGAVELSWDYMQSDLGELPVDARFPPRVPKSFPFNTSVVYKKTLFVEFTD |
| | HLFNIAKPRPPWMGLLGPTIQAEVYDTVVITLKNMASHPVSLHAVGVSYWKASEGAEY |
| | DDQTSQREKEDDKVFPGGSHTYVWQVLKENGPMASDPLCLTYSYLSHVDLVKDLNSG |
| | LIGALLVCREGSLAKEKTQTLHKFILLFAVFDEGKSWHSETKNSLMQDRDAASARAWP |
| | KMHTVNGYVNRSLPGLIGCHRKSVYWHVIGMGTTPEVHSIFLEGHTFLVRNHRQASLEI |
| | SPITFLTAQTLLMDLGQFLLFCHISSHQHDGMEAYVKVDSCPEEPQLRMKNNEEAEDYD |
| | DDLTDSEMDVVRFDDDNSPSFIQIRSVAKKHPKTWVHYIAAEEEDWDYAPLVLAPDDR |
| | SYKSQYLNNGPQRIGRKYKKVRFMAYTDETFKTREAIQHESGILGPLLYGEVGDTLLIIF |
| | KNQASRPYNIYPHGITDVRPLYSRRLPKGVKHLKDFPILPGEIFKYKWTVTVEDGPTKSD |
| | PRCLTRYYSSFVNMERDLASGLIGPLLICYKESVDQRGNQIMSDKRNVILFSVFDENRSW |
| | YLTENIQRFLPNPAGVQLEDPEFQASNIMHSINGYVFDSLQLSVCLHEVAYWYILSIGAQ |
| | TDFLSVFFSGYTFKHKMVYEDTLTLFPFSGETVFMSMENPGLWILGCHNSDFRNRGMTA |
| | LLKVSSCDKNTGDYYEDSYEDISAYLLSKNNAIEPRSFSQNPPVLKRHQAEITRTTLQSD |
| | QEEIDYDDTISVEMKKEDFDIYDEDENQSPRSFQKKTRHYFIAAVERLWDYGMSSSPHV |
| | LRNRAQSGSVPQFKKVVFQEFTDGSFTQPLYRGELNEHLGLLGPYIRAEVEDNIMVTFR |
| | NQASRPYSFYSSLISYEEDQRQGAEPRKNFVKPNETKTYFWKVQHHMAPTKDEFDCKA |
| | WAYFSDVDLEKDVHSGLIGPLLVCHTNTLNPAHGRQVTVQEFALFFTIFDETKSWYFTE |
| | NMERNCRAPCNIQMEDPTFKENYRFHAINGYIMDTLPGLVMAQDQRIRWYLLSMGSNE |
| | NIHSIHFSGHVFTVRKKEEYKMALYNLYPGVFETVEMLPSKAGIWRVECLIGEHLHAGM |
| | STLFLVYSNKCQTPLGMASGHIRDFQITASGQYGQWAPKLARLHYSGSINAWSTKEPFS |
| | WIKVDLLAPMIIHGIKTQGARQKFSSLYISQFIIMYSLDGKKWQTYRGNSTGTLMVFFGN |
| | VDSSGIKHNIFNPPIIARYIRLHPTHYSIRSTLRMELMGCDLNSCSMPLGMESKAISDAQIT |
| | ASSYFTNMFATWSPSKARLHLQGRSNAWRPQVNNPKEWLQVDFQKTMKVTGVTTQG |
| | VKSLLTSMYVKEFLISSSQDGHQWTLFFQNGKVKVFQGNQDSFTPVVNSLDPPLLTRYL |
| | RIHPQSWVHQIALRMEVLGCEAQDLY |

1-111. (canceled)

112. A fusion protein comprising a factor VIII (FVIII) polypeptide fused to an extended recombinant polypeptide (XTEN), wherein the XTEN comprises one or more XTEN sequence motifs, and
wherein the XTEN is further characterized in that (a) the sum of glycine (G), alanine (A), serine (S), threonine (T), glutamate (E) and proline (P) residues constitutes more than about 80% of the total amino acid residues of the XTEN and (b) the XTEN contains no three contiguous amino acids that are identical unless the amino acids are serine.

113. The fusion protein of claim 112, wherein one or more XTEN sequence motif is selected from SEQ ID NOs: 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49.

114. The fusion protein of claim 112, wherein the XTEN comprises at least 36 amino acids.

115. The fusion protein of claim 112, wherein the XTEN comprises an amino acid sequence at least 90% identical to SEQ ID NOs: 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, and 592.

116. The fusion protein of claim 115, wherein the XTEN comprises an amino acid sequence at least 95% identical to SEQ ID NOs: 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, and 592.

117. The fusion protein of claim 115, wherein the XTEN comprises an amino acid sequence identical to SEQ ID NOs: 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, and 592.

118. The fusion protein of claim 112, wherein the XTEN comprises an amino acid sequence at least 90% identical to SEQ ID NO: 60, SEQ ID NO: 66, SEQ ID NO: 73, SEQ ID NO: 76, SEQ ID NO: 78, or SEQ ID NO: 82.

119. The fusion protein of claim 118, wherein the XTEN comprises an amino acid sequence at least 95% identical to SEQ ID NO: 60, SEQ ID NO: 66, SEQ ID NO: 73, SEQ ID NO: 76, SEQ ID NO: 78, or SEQ ID NO: 82.

120. The fusion protein of claim 118, wherein the XTEN comprises an amino acid sequence identical to SEQ ID NO: 60, SEQ ID NO: 66, SEQ ID NO: 73, SEQ ID NO: 76, SEQ ID NO: 78, or SEQ ID NO: 82.

121. The fusion protein of claim 112, wherein the FVIII polypeptide comprises human FVIII.

122. The fusion protein of claim 112, wherein the FVIII polypeptide comprises FVIII with all or a portion of B domain deleted.

123. The fusion protein of claim 112, which exhibits a prolonged half-life compared to a FVIII polypeptide not linked to the XTEN.

124. The fusion protein of claim 112, which further comprises a heterologous polypeptide.

125. The fusion protein of claim 112, further comprising a cleavage sequence between the FVIII polypeptide and the XTEN.

126. The fusion protein of claim 125, wherein the cleavage sequence is cleavable by a mammalian protease selected from FXIa, FXIIa, kallikrein, FVIIIa, FXa, FIIa (thrombin), Elastase-2, granzyme B, MMP-12, MMP-13, MMP-17 or MMP-20.

127. A pharmaceutical composition comprising the fusion protein of claim 112 and a pharmaceutically acceptable carrier.

128. A method of treating coagulopathy in a subject, comprising administering to the subject a composition comprising a therapeutically effective amount of the fusion protein of claim 112.

129. A method of controlling or ameliorating a bleeding episode in a subject in need thereof comprising administering to the subject a therapeutically effective amount of the fusion protein of claim 112.

130. A nucleic acid molecule encoding the fusion protein of claim 112.

131. A method of making a fusion protein comprising culturing a host cell comprising the nucleic acid molecule of claim 130 under suitable conditions.

132. The method of claim 131, further comprising recovering the fusion protein.
